(19) United States
(12) Patent Application Publication
(10) Pub. No.: US 2005/0143817 A1
(43) Pub. Date: Jun. 30, 2005

(54) Medical implants and anti-scarring agents

(75) Inventors: William L. Hunter, Vancouver (CA); David M. Gravett, Vancouver (CA); Philip M. Toleikis, Vancouver (CA); Arpita Maiti, Vancouver (CA); Pierre E. Signore, Vancouver (CA); Richard T. Liggins, Coquitlam (CA)

Correspondence Address: SEED INTELLECTUAL PROPERTY LAW GROUP PLLC, 701 FIFTH AVENYUE, SUITE 6300, SEATTLE, WA, 98104-7092, US

(73) Assignee: Angiotech International AG, Zug (CH)

(21) Appl. No.: 11/006,899

(22) Filed: Dec. 7, 2004

Related U.S. Application Data
(63) Continuation of application No. 10/986,231, filed on Nov. 10, 2004.
(60) Provisional application No. 60/586,861, filed on Jul. 9, 2004.
(60) Provisional application No. 60/578,471, filed on Jun. 9, 2004.
(60) Provisional application No. 60/526,541, filed on Dec. 3, 2003.
(60) Provisional application No. 60/525,226, filed on Nov. 24, 2003.
(60) Provisional application No. 60/523,908, filed on Nov. 20, 2003.
(60) Provisional application No. 60/524,023, filed on Nov. 20, 2003.
(60) Provisional application No. 60/518,785, filed on Nov. 10, 2003.

(51) Int. Cl. A61F 2/02

(52) U.S. Cl. 623/11.11; 623/926

(57) ABSTRACT

Implants are used in combination with an anti-scarring agent in order to inhibit scarring that may otherwise occur when the implant is placed within an animal. The agent may be any suitable anti-scarring agent, e.g., a cell cycle inhibitor, and may be used in conjunction with a second pharmaceutical agent, e.g., an antibiotic. Suitable implants include intravascular implants, a vascular graft or wrap implant, an implant for hemodialysis access, an implant that provides an anastomotic connection, ventricular assist implant, a prosthetic heart valve implant, an inferior vena cava filter implant, a peritoneal dialysis catheter implant, a central nervous system shunt, an intraocular lens, an implant for glaucoma drainage, a penile implant, an endotracheal tube, a tracheostomy tube, a gastrointestinal device, and a spinal implant.

### CROSS-REFERENCE TO RELATED APPLICATIONS

This application is a Continuation of co-pending U.S. Utility application Ser. No. 10/986,231, filed Nov. 10, 2004, which claims the benefit under 35 U.S.C. § 119(e) of U.S. Provisional Application Ser. No. 60/518,785, filed Nov. 10, 2003; U.S. Provisional Application Ser. No. 60/523,908, filed Nov. 20, 2003; U.S. Provisional Application Ser. No. 60/524,023, filed Nov. 20, 2003; U.S. Provisional Application Ser. No. 60/525,226, filed Nov. 24, 2003; U.S. Provisional Application Ser. No. 60/526,541, filed Dec. 3, 2003; U.S. Provisional Application Ser. No. 60/586,861, filed Jul. 9, 2004; and U.S. Provisional Application Ser. No. 60/578,471, filed Jun. 9, 2004, which applications are incorporated herein by reference in their entireties.

### BACKGROUND OF THE INVENTION

1. Field of the Invention

The present invention relates generally to pharmaceutical compositions, methods and devices, and more specifically, to compositions and methods for preparing and using medical implants to make them resistant to overgrowth by inflammatory and fibrous scar tissue.

2. Description of the Related Art

The clinical function of numerous medical implants and devices is dependent upon the device being able to effectively maintain an anatomical, or surgically created, space or passageway. Unfortunately, many devices implanted in the body are subject to a “foreign body” response from the surrounding host tissues. In particular, injury to tubular anatomical structures (such as blood vessels, the gastrointestinal tract, the male and female reproductive tract, the urinary tract, sinuses, spinal nerve root canals, lacrimal ducts, Eustachian tubes, the auditory canal, and the respiratory tract) from surgery and/or injury created by the implantation of medical devices can lead to a well known clinical problem called “stenosis” (or narrowing). Stenosis occurs in response to trauma to the epithelial lining or the entire body tube during the procedure, including virtually any manipulation which attempts to relieve obstruction of the passageway, and is a major factor limiting the effectiveness of invasive treatments for a variety of diseases to be described later.

Stenosis (or “restenosis” if the problem recurs after an initially successful attempt to open a blocked passageway) is a form of response to injury leading to wall thickening, narrowing of the lumen, and loss of function in the tissue supplied by the particular passageway. Physical injury during an interventional procedure results in damage to epithelial lining of the tube and the smooth muscle cells (SMCs) that make up the wall. The damaged cells, particularly SMCs, release cytokines, which recruit inflammatory cells such as macrophages, lymphocytes and neutrophils (i.e., which are some of the known white blood cells) into the area. The white blood cells in turn release a variety of additional cytokines, growth factors, and tissue degrading enzymes that influence the behavior of the constituent cells of the wall (primarily epithelial cells and SMCs). Stimulation of the SMCs induces them to migrate into the inner aspect of the body passageway (often called the “intima”), proliferate and secrete an extracellar matrix—effectively filling all or parts of the lumen with reactive, fibrous scar tissue. Collectively, this creates a thickening of the intimal layer (known in some tissues as “neointimal hyperplasia”) that narrows the lumen of the passageway and can be significant enough to obstruct its lumen.

The present invention discloses pharmaceutical agents which inhibit one or more aspects of the production of excessive fibrous (scar) tissue. Furthermore, compositions and methods are described for coating medical devices and implants with drug-delivery compositions such that the pharmaceutical agent is delivered in therapeutic levels over a period sufficient to allow normal healing to occur. And finally, numerous specific implants and devices are described that produce superior clinical results as a result of being coated with agents that reduce excessive scarring and fibrous tissue accumulation as well as other related advantages.

### BRIEF SUMMARY OF THE INVENTION

Briefly stated, in one aspect, the present invention provides compositions for delivery of selected therapeutic agents via medical implants or implantable medical devices, as well as methods for making and using these implants and devices. Within one aspect of the invention, drug-coated or drug-impregnated implants and medical devices are provided which reduce fibrosis in the tissue surrounding the device or implant, or inhibit scar development on the device/implant surface, thus enhancing the efficacy the procedure. Within various embodiments, fibrosis is inhibited by local or systemic release of specific pharmacological agents that become localized to the adjacent tissue.

The repair of tissues following a mechanical or surgical intervention involves two distinct processes: (1) regeneration (the replacement of injured cells by cells of the same type and (2) fibrosis (the replacement of injured cells by connective tissue). There are four general components to the process of fibrosis (or scarring) including: formation of new blood vessels (angiogenesis), migration and proliferation of connective tissue cells (such as fibroblasts or smooth muscle cells), deposition of extracellular matrix (ECM), and remodeling (maturation and organization of the fibrous tissue). Within one embodiment of the invention, an implant or device is adapted to release an agent that inhibits fibrosis or regeneration through one or more of the mechanisms sited herein.

Within yet other aspects of the present invention, methods are provided for manufacturing a medical device or implant, comprising the step of coating (e.g., spraying, dipping, wrapping, or administering drug through) a medical device or implant. Additionally, the implant or medical device can be constructed so that the device itself is comprised of materials which inhibit fibrosis in or around the implant. A wide variety of medical devices and implants may be utilized within the context of the present invention, depending on the site and nature of treatment desired.

Within related aspects of the present invention, vascular stents, gastrointestinal stents, tracheal/bronchial stents, genital-urinary stents, ENT stents, intraocular lenses, implants for hypertrophic scars and keloids, vascular grafts, anastomotic connector devices, surgical adhesion barriers, glaucoma drainage devices, prosthetic heart valves, tympanostomy tubes, penile implants, CVCs, ventricular assist devices (e.g., LVAD's), spinal prostheses, endotracheal and tracheostomy tubes, peritoneal dialysis catheters, intracranial pressure monitors, vena cava filters, and gastrointestinal drainage tubes are provided comprising an implant or device, wherein the implant or device is in combination with an agent which inhibits fibrosis in vivo.

Within various embodiments of the invention, the implant or device is further coated with a composition or compound, which delays the onset of activity of the fibrosis-inhibiting agent for a period of time after implantation. Representative examples of such agents include heparin, PLGA/MePEG, PLA, and polyethylene glycol. Within further embodiments the fibrosis-inhibiting implant or device is activated before, during, or after deployment (e.g., an inactive agent on the device is first activated to one that reduces or inhibits an in vivo fibrotic reaction).

Within various embodiments of the invention, a device or implant is coated on one aspect, portion or surface with a composition which inhibits fibrosis, as well as being coated with a composition or compound which promotes scarring on another aspect, portion or surface of the device. Representative examples of agents that promote fibrosis and scarring include silk, wool, silica, bleomycin, neomycin, talcum powder, metallic beryllium, and copper as well as analogues and derivatives thereof.

Also provided by the present invention are methods for treating patients undergoing surgical, endoscopic or minimally invasive therapies where a medical device or implant is placed as part of the procedure. As utilized herein, it should be understood that “inhibits fibrosis or stenosis” refers to a statistically significant decrease in the amount of scar tissue in or around the device or an improvement in the luminal area of the device/implant, which may or may not result in a permanent prohibition of any complications or failures of the device/implant.

The pharmaceutical agents and compositions are utilized to create novel drug-coated implants and medical devices that reduce the foreign body response to implantation and limit the growth of reactive tissue on the surface of, or around in the tissue surrounding the device, such that performance is enhanced. In many instances, the devices are used to maintain body lumens or passageways such as blood vessels, the gastrointestinal tract, the male and female reproductive tract, the urinary tract, bony foramena (e.g., sinuses, spinal nerve root canals, lacrimal ducts, Eustachian tubes, the auditory canal), and the respiratory tract, where obstruction of the device by scar tissue in the post-procedural period leads to the adverse clinical sequela or failure of the intervention. Medical devices and implants coated with selected pharmaceutical agents designed to prevent scar tissue overgrowth and preserve patency can offer significant clinical advantages over uncoated devices.

For example, in one aspect the present invention is directed to devices that comprise a medical implant and at least one of (i) an anti-scarring agent and (II) a composition that comprises an anti-scarring agent. The agent is present so as to inhibit scarring that can otherwise occur when the implant is placed within an animal. In another aspect the present invention is directed to methods wherein both an implant and at least one of (i) an anti-scarring agent and (II) a composition that comprises an anti-scarring agent, are placed into an animal, and the agent inhibits scarring that can otherwise occur. These and other aspects of the invention are summarized below.

Thus, in various independent aspects, the present invention provides the following: a device, comprising a gastrointestinal implant and an anti-scarring agent or a composition comprising an anti-scarring agent, wherein the agent inhibits scarring; a device, comprising an inferior vena cava filter implant and an anti-scarring agent or a composition comprising an anti-scarring agent, wherein the agent inhibits scarring; a device, comprising a central nervous system shunt implant and an anti-scarring agent or a composition comprising an anti-scarring agent, wherein the agent inhibits scarring; a device, comprising a pressure monitoring implant and an anti-scarring agent or a composition comprising an anti-scarring agent, wherein the agent inhibits scarring; a device, comprising a peritoneal dialysis catheter implant and an anti-scarring agent or a composition comprising an anti-scarring agent, wherein the agent inhibits scarring; a device, comprising an endotracheal tube implant and an anti-scarring agent or a composition comprising an anti-scarring agent, wherein the agent inhibits scarring; a device, comprising a tracheostomy tube implant and an anti-scarring agent or a composition comprising an anti-scarring agent, wherein the agent inhibits scarring; a device, comprising a penile implant and an anti-scarring agent or a composition comprising an anti-scarring agent, wherein the agent inhibits scarring; a device, comprising a tympanostomy tube implant and an anti-scarring agent or a composition comprising an anti-scarring agent, wherein the agent inhibits scarring; device, comprising a prosthetic heart valve implant and an anti-scarring agent or a composition comprising an anti-scarring agent, wherein the agent inhibits scarring; a device, comprising a glaucoma drainage implant and an anti-scarring agent or a composition comprising an anti-scarring agent, wherein the agent inhibits scarring; a device, comprising an implant that provides a surgical adhesion barrier and an anti-scarring agent or a composition comprising an anti-scarring agent, wherein the agent inhibits scarring; a device, comprising an anastomotic connector implant and an anti-scarring agent or a composition comprising an anti-scarring agent, wherein the agent inhibits scarring; a device, comprising a sensing implant and an anti-scarring agent or a composition comprising an anti-scarring agent, wherein the agent inhibits scarring; a device, comprising an implant for pericardial treatment of coronary artery disease and an anti-scarring agent or a composition comprising an anti-scarring agent, wherein the agent inhibits scarring; a device, comprising vascular graft implant and an anti-scarring agent or a composition comprising an anti-scarring agent, wherein the agent inhibits scarring; a device, comprising an implant for the treatment of a hypertrophic scar and an anti-scarring agent or a composition comprising an anti-scarring agent, wherein the agent inhibits scarring; a device, comprising an implant for the treatment of a keloid and an anti-scarring agent or a composition comprising an anti-scarring agent, wherein the agent inhibits scarring; a device, comprising an intraocular lens implant and an anti-scarring agent or a composition comprising an anti-scarring agent, wherein the agent inhibits scarring; a device, comprising an ENT stent implant and an anti-scarring agent or a composition comprising an anti-scarring agent, wherein the agent inhibits scarring; a device, comprising an genital-urinary stent implant and an anti-scarring agent or a composition comprising an anti-scarring agent, wherein the agent inhibits scarring; a device, comprising a tracheal/bronchial stent implant and an anti-scarring agent or a composition comprising an anti-scarring agent, wherein the agent inhibits scarring, a device, comprising GI stent implant and an anti-scarring agent or a composition comprising an anti-scarring agent, wherein the agent inhibits scarring. These and other devices are described in more detail herein.

In each of the aforementioned devices, in separate aspects the present invention provides that: the agent is a cell cycle inhibitor; the agent is an anthracycline; the agent is a taxane; the agent is a podophyllotoxin; the agent is an immunomodulator; the agent is a heat shock protein 90 antagonist; the agent is a HMGCoA reductase inhibitor; the agent is an inosine monophosphate dehydrogenase inhibitor; the agent is an NF kappa B inhibitor; the agent is a p38 MAP kinase inhibitor. These and other agents are described in more detail herein.

In additional aspects, for each of the aforementioned devices combined with each of the aforementioned agents, it is, for each combination, independently disclosed that the agent may be present in a composition along with a polymer. In one embodiment of this aspect, the polymer is biodegradable. In another embodiment of this aspect, the polymer is non-biodegradable. Other features and characteristics of the polymer, which may serve to describe the present invention for every combination of device and agent described above, are set forth in greater detail herein.

In addition to devices, the present invention also provides methods. For example, in additional aspects of the present invention, for each of the aforementioned devices, and for each of the aforementioned combinations of the devices with the anti-scarring agents, the present invention provides methods whereby a specified device is implanted into an animal, and a specified agent associated with the device inhibits scarring that can otherwise occur. Each of the devices identified herein may be a “specified device”, and each of the anti-scarring agents identified herein may be an “anti-scarring agent”, where the present invention provides, in independent embodiments, for each possible combination of the device and the agent.

The agent may be associated with the device prior to the device being placed within the animal. For example, the agent (or composition comprising the agent) may be coated onto an implant, and the resulting device then placed within the animal. In addition, or alternatively, the agent may be independently placed within the animal in the vicinity of where the device is to be, or is being, placed within the animal. For example, the agent may be sprayed or otherwise placed onto the tissue that will be contacting the medical implant or may otherwise undergo scarring. To this end, the present invention provides, in independent aspects: a method for inhibiting scarring comprising placing a gastrointestinal implant and an anti-scarring agent or a composition comprising an anti-scarring agent into an animal host, wherein the agent inhibits scarring; a method for inhibiting scarring comprising placing an inferior vena cava filter implant and an anti-scarring agent or a composition comprising an anti-scarring agent into an animal host, wherein the agent inhibits scarring; a method for inhibiting scarring comprising placing a central nervous system shunt implant and an anti-scarring agent or a composition comprising an anti-scarring agent into an animal host, wherein the agent inhibits scarring; a method for inhibiting scarring comprising placing a pressure monitoring implant and an anti-scarring agent or a composition comprising an anti-scarring agent into an animal host, wherein the agent inhibits scarring; a method for inhibiting scarring comprising placing a peritoneal dialysis catheter implant and an anti-scarring agent or a composition comprising an anti-scarring agent into an animal host, wherein the agent inhibits scarring; a method for inhibiting scarring comprising placing an endotracheal tube implant and an anti-scarring agent or a composition comprising an anti-scarring agent into an animal host, wherein the agent inhibits scarring; a method for inhibiting scarring comprising placing a tracheostomy tube implant and an anti-scarring agent or a composition comprising an anti-scarring agent into an animal host, wherein the agent inhibits scarring; a method for inhibiting scarring comprising placing a penile implant and an anti-scarring agent or a composition comprising an anti-scarring agent into an animal host, wherein the agent inhibits scarring; a method for inhibiting scarring comprising placing a tympanostomy tube implant and an anti-scarring agent or a composition comprising an anti-scarring agent into an animal host, wherein the agent inhibits scarring; a method for inhibiting scarring comprising placing a prosthetic heart valve implant and an anti-scarring agent or a composition comprising an anti-scarring agent into an animal host, wherein the agent inhibits scarring; a method for inhibiting scarring comprising placing a glaucoma drainage implant and an anti-scarring agent or a composition comprising an anti-scarring agent into an animal host, wherein the agent inhibits scarring; a method for inhibiting scarring comprising placing a pressure monitoring implant and an anti-scarring agent or a composition comprising an anti-scarring agent into an animal host, wherein the agent inhibits scarring; a method for inhibiting scarring comprising placing a drug delivery pump implant and an anti-scarring agent or a composition comprising an anti-scarring agent into an animal host, wherein the agent inhibits scarring; a method for inhibiting scarring comprising placing an anastomotic connector implant and an anti-scarring agent or a composition comprising an anti-scarring agent into an animal host, wherein the agent inhibits scarring; a method for inhibiting scarring comprising placing a sensing implant and an anti-scarring agent or a composition comprising an anti-scarring agent into an animal host, wherein the agent inhibits scarring; a method for inhibiting scarring comprising placing an implant for pericardial treatment of coronary artery disease and an anti-scarring agent or a composition comprising an anti-scarring agent into an animal host, wherein the agent inhibits scarring; a method for inhibiting scarring comprising placing a vascular graft implant and an anti-scarring agent or a composition comprising an anti-scarring agent into an animal host, wherein the agent inhibits scarring; a method for inhibiting scarring comprising placing an implant for the treatment of a hypertrophic scar and an anti-scarring agent or a composition comprising an anti-scarring agent into an animal host, wherein the agent inhibits scarring; a method for inhibiting scarring comprising placing an implant for the treatment of a keloid and an anti-scarring agent or a composition comprising an anti-scarring agent into an animal host, wherein the agent inhibits scarring; a method for inhibiting scarring comprising placing an intraocular lens implant and an anti-scarring agent or a composition comprising an anti-scarring agent into an animal host, wherein the agent inhibits scarring; a method for inhibiting scarring comprising placing an ENT stent implant and an anti-scarring agent or a composition comprising an anti-scarring agent into an animal host, wherein the agent inhibits scarring; a method for inhibiting scarring comprising placing a genital-urinary stent implant and an anti-scarring agent or a composition comprising an anti-scarring agent into an animal host, wherein the agent inhibits scarring; a method for inhibiting scarring comprising placing a tracheal/bronchial stent implant and an anti-scarring agent or a composition comprising an anti-scarring agent into an animal host, wherein the agent inhibits scarring; a method for inhibiting scarring comprising placing a GI stent implant and an anti-scarring agent or a composition comprising an anti-scarring agent into an animal host, wherein the agent inhibits scarring In each of the aforementioned methods, in separate aspects, the present invention provides that: the agent is a cell cycle inhibitor; the agent is an anthracycline; the agent is a taxane; the agent is a podophyllotoxin; the agent is an immunomodulator; the agent is a heat shock protein 90 antagonist; the agent is a HMGCoA reductase inhibitor; the agent is an inosine monophosphate dehydrogenase inhibitor; the agent is an NF kappa B inhibitor; the agent is a p38 MAP kinase inhibitor. These and other agents are described in more detail herein.

In additional aspects, for each of the aforementioned methods used in combination with each of the aforementioned agents, it is, for each combination, independently disclosed that the agent may be present in a composition along with a polymer. In one embodiment of this aspect, the polymer is biodegradable. In another embodiment of this aspect, the polymer is non-biodegradable. Other features and characteristics of the polymer, which may serve to describe the present invention for every combination of device and agent described above, are set forth in greater detail herein.

These and other aspects of the present invention will become evident upon reference to the following detailed description and attached drawings. In addition, various references are set forth herein which describe in more detail certain procedures and/or compositions (e.g., polymers), and are therefore incorporated by reference in the entirety.

### BRIEF DESCRIPTION OF THE DRAWINGS

FIG. 1 is a diagram showing how a cell cycle inhibitor acts at one or more of the steps in the biological pathway.

FIG. 2 is graph showing the results of a screening assay for assessing the effect of mitoxantrone (mitoxantrone IC₅₀=20 nM) on proliferation of human fibroblasts.

FIG. 3 is a picture that shows an uninjured carotid artery from a rat balloon injury model.

FIG. 4 is a picture that shows an injured carotid artery from a rat balloon injury model.

FIG. 5 is a picture that shows a paclitaxel/mesh treated carotid artery in a rat balloon injury model (345 μg paclitaxel in a 50:50 PLG coating on a 10:90 PLG mesh).

FIG. 6A schematically depicts the transcriptional regulation of matrix metalloproteinases.

FIG. 6B is a blot which demonstrates that IL-1 stimulates AP-1 transcriptional activity.

FIG. 6C is a graph which shows that IL-1 induced binding activity decreased in lysates from chondrocytes which were pretreated with paclitaxel.

FIG. 6D is a blot which shows that IL-1 induction increases collagenase and stromelysin in RNA levels in chondrocytes, and that this induction can be inhibited by pretreatment with paclitaxel.

FIGS. 7A-H are blots that show the effect of various anti-microtubule agents in inhibiting collagenase expression.

FIG. 8 is a graph showing the results of a screening assay for assessing the effect of paclitaxel on smooth muscle cell migration (paclitaxel IC₅₀=0.76 nM).

FIG. 9 is a graph showing the results of a screening assay for assessing the effect of geldanamycin on IL-1β production by macrophages (IC₅₀=20 nM for IL-1β production by THP-1 cells).

FIG. 10 is a graph showing the results of a screening assay for assessing the effect of geldanamycin on IL-8 production by macrophages (IC₅₀=27 nM for IL-8 production by THP-1 cells).

FIG. 11 is a graph showing the results of a screening assay for assessing the effect of geldanamycin on MCP-1 production by macrophages (IC₅₀=7 nM for MCP-1 production by THP-1 cells).

FIG. 12 is a graph showing the results for the screening assay for assessing the effect of mitoxantrone on nitric oxide production by macrophages.

FIG. 13 is a graph showing the results for the screening assay for assessing the effect of various therapeutic agents on TNF-alpha production by macrophages.

FIG. 14 is graph showing the results of a screening assay for assessing the effect of rapamycin on cell proliferation of human fibroblasts.

FIG. 15 is a graph showing the results for the screening assay for assessing the effect of rapamycin concentration for TNFα production by THP-1 cells.

FIG. 16 is graph showing the results of a screening assay for assessing the effect of paclitaxel on proliferation of smooth muscle cells.

FIG. 17 is graph showing the results of a screening assay for assessing the effect of paclitaxel on cell proliferation of human fibroblasts.

FIG. 18 is graph showing the results of a screening assay for assessing the effect of paclitaxel (IC₅₀=1 34 nM) for proliferation of the murine RAW 264.7 macrophage cell line.

### DETAILED DESCRIPTION OF THE INVENTION

Definitions

Prior to setting forth the invention, it may be helpful to an understanding thereof to first set forth definitions of certain terms that are used herein.

Any concentration ranges, percentage range, or ratio range recited herein are to be understood to include concentrations, percentages or ratios of any integer within that range and fractions thereof, such as one tenth and one hundredth of an integer, unless otherwise indicated. Also, any number range recited herein relating to any physical feature, such as polymer subunits, size or thickness, are to be understood to include any integer within the recited range, unless otherwise indicated. It should be understood that the terms “a” and “an” as used above and elsewhere herein refer to “one or more” of the enumerated components. For example, “a” polymer refers to both one polymer or a mixture comprising two or more polymers As used herein, the term “about” means±15%.

“Fibrosis,” “Scarring,” or “Fibrotic Response” refers to the formation of fibrous tissue in response to injury or medical intervention. Therapeutic agents which inhibit fibrosis or scarring are referred to herein as “fibrosis-inhibiting agents”, “anti-scarring agents”, and the like, where these agents inhibit fibrosis through one or more mechanisms including: inhibiting angiogenesis, inhibiting migration or proliferation of connective tissue cells (such as fibroblasts, smooth muscle cells, vascular smooth muscle cells), reducing ECM production, and/or inhibiting tissue remodeling.

“Host”, “Person”, “Subject”, “Patient” and the like are used synonymously to refer to the living being into which a device of the present invention is implanted.

“Implanted” refers to having completely or partially placed a device within a host. A device is partially implanted when some of the device reaches, or extends to the outside of, a host.

“Inhibit fibrosis”, “reduce fibrosis” and the like are used synonymously to refer to the action of agents or compositions which result in a statistically significant decrease in the formation of fibrous tissue that can be expected to occur in the absence of the agent or composition.

“Inhibitor” refers to an agent which prevents a biological process from occurring or slows the rate or degree of occurrence of a biological process. The process may be a general one such as scarring or refer to a specific biological action such as, for example, a molecular process resulting in release of a cytokine.

“Antagonist” refers to an agent which prevents a biological process from occurring or slows the rate or degree of occurrence of a biological process. While the process may be a general one, typically this refers to a drug mechanism where the drug competes with a molecule for an active molecular site or prevents a molecule from interacting with the molecular site. In these situations, the effect is that the molecular process is inhibited.

“Agonist” refers to an agent which stimulates a biological process or rate or degree of occurrence of a biological process. The process may be a general one such as scarring or refer to a specific biological action such as, for example, a molecular process resulting in release of a cytokine.

“Anti-microtubule Agents” should be understood to include any protein, peptide, chemical, or other molecule which impairs the function of microtubules, for example, through the prevention or stabilization of polymerization. Compounds that stabilize polymerization of microtubules are referred to herein as “microtubule stabilizing agents.” A wide variety of methods may be utilized to determine the anti-microtubule activity of a particular compound, including for example, assays described by Smith et al. (Cancer Lett 79(2): 213-219, 1994) and Mooberry et al., (Cancer Lett. 96(2): 261-266, 1995).

“Medical Device”, “Implant”, “Medical Device or Implant”, “implant/device” and the like are used synonymously to refer to any object that is designed to be placed partially or wholly within a patient's body for one or more therapeutic or prophylactic purposes such as for restoring physiological function, alleviating symptoms associated with disease, delivering therapeutic agents, and/or repairing or replacing or augmenting etc. damaged or diseased organs and tissues. While normally composed of biologically compatible synthetic materials (e.g., medical-grade stainless steel, titanium and other metals; polymers such as polyurethane, silicon, PLA, PLGA and other materials) that are exogenous, some medical devices and implants include materials derived from animals (e.g., “xenografts” such as whole animal organs; animal tissues such as heart valves; naturally occurring or chemically-modified molecules such as collagen; hyaluronic acid, proteins, carbohydrates and others), human donors (e.g., “allografts” such as whole organs; tissues such as bone grafts, skin grafts and others), or from the patients themselves (e.g., “autografts” such as saphenous vein grafts, skin grafts, tendon/ligament/muscle transplants). Medical devices of particular utility in the present invention include, but are not restricted to, vascular stents, gastrointestinal stents, tracheal/bronchial stents, genital-urinary stents, ENT stents, intraocular lenses, implants for hypertrophic scars and keloids, vascular grafts, anastomotic connector devices, surgical adhesion barriers, glaucoma drainage devices, film or mesh, prosthetic heart valves, tympanostomy tubes, penile implants, endotracheal and tracheostomy tubes, peritoneal dialysis catheters, intracranial pressure monitors, vena cava filters, CVCs, ventricular assist device (e.g., LVAD), spinal prostheses, and gastrointestinal drainage tubes.

“Release of an agent” refers to a statistically significant presence of the agent, or a subcomponent thereof, which has disassociated from the implant/device.

“Biodegradable” refers to materials for which the degradation process is at least partially mediated by, and/or performed in, a biological system.

“Degradation” refers to a chain scission process by which a polymer chain is cleaved into oligomers and monomers. Chain scission may occur through various mechanisms, including, for example, by chemical reaction (e.g., hydrolysis) or by a thermal or photolytic process. Polymer degradation may be characterized, for example, using gel permeation chromatography (GPC), which monitors the polymer molecular mass changes during erosion and drug release. Biodegradable also refers to materials may be degraded by an erosion process mediated by, and/or performed in, a biological system. “Erosion” refers to a process in which material is lost from the bulk. In the case of a polymeric system, the material may be a monomer, an oligomer, a part of a polymer backbone, or a part of the polymer bulk. Erosion includes (i) surface erosion, in which erosion affects only the surface and not the inner parts of a matrix; and (II) bulk erosion, in which the entire system is rapidly hydrated and polymer chains are cleaved throughout the matrix. Depending on the type of polymer, erosion generally occurs by one of three basic mechanisms (see, e.g., Heller, J., CRC Critical Review in Therapeutic Drug Carrier Systems (1984), 1(1), 39-90); Siepmann, J. et al., Adv. Drug Del. Rev. (2001), 48, 229-247): (1) water-soluble polymers that have been insolubilized by covalent cross-links and that solubilize as the cross-links or the backbone undergo a hydrolytic cleavage; (2) polymers that are initially water insoluble are solubilized by hydrolysis, ionization, or pronation of a pendant group; and (3) hydrophobic polymers are converted to small water-soluble molecules by backbone cleavage. Techniques for characterizing erosion include thermal analysis (e.g., DSC), X-ray diffraction, scanning electron microscopy (SEM), electron paramagnetic resonance spectroscopy (EPR), NMR imaging, and recording mass loss during an erosion experiment. For microspheres, photon correlation spectroscopy (PCS) and other particles size measurement techniques may be applied to monitor the size evolution of erodible devices versus time.

As used herein, “analogue” refers to a chemical compound that is structurally similar to a parent compound, but differs slightly in composition (e.g., one atom or functional group is different, added, or removed). The analogue may or may not have different chemical or physical properties than the original compound and may or may not have improved biological and/or chemical activity. For example, the analogue may be more hydrophilic or it may have altered reactivity as compared to the parent compound. The analogue may mimic the chemical and/or biologically activity of the parent compound (i.e., it may have similar or identical activity), or, in some cases, may have increased or decreased activity. The analogue may be a naturally or non-naturally occurring (e.g., recombinant) variant of the original compound. An example of an analogue is a mutein (i.e., a protein analogue in which at least one amino acid is deleted, added, or substituted with another amino acid). Other types of analogues include isomers (enantiomers, diasteromers, and the like) and other types of chiral variants of a compound, as well as structural isomers. The analogue may be a branched or cyclic variant of a linear compound. For example, a linear compound may have an analogue that is branched or otherwise substituted to impart certain desirable properties (e.g., improve hydrophilicity or bioavailability).

As used herein, “derivative” refers to a chemically or biologically modified version of a chemical compound that is structurally similar to a parent compound and (actually or theoretically) derivable from that parent compound. A “derivative” differs from an “analogue” in that a parent compound may be the starting material to generate a “derivative,” whereas the parent compound may not necessarily be used as the starting material to generate an “analogue.” A derivative may or may not have different chemical or physical properties of the parent compound. For example, the derivative may be more hydrophilic or it may have altered reactivity as compared to the parent compound. Derivatization (i.e., modification) may involve substitution of one or more moieties within the molecule (e.g., a change in functional group). For example, a hydrogen may be substituted with a halogen, such as fluorine or chlorine, or a hydroxyl group (—OH) may be replaced with a carboxylic acid moiety (—COOH). The term “derivative” also includes conjugates, and prodrugs of a parent compound (i.e., chemically modified derivatives which can be converted into the original compound under physiological conditions). For example, the prodrug may be an inactive form of an active agent. Under physiological conditions, the prodrug may be converted into the active form of the compound. Prodrugs may be formed, for example, by replacing one or two hydrogen atoms on nitrogen atoms by an acyl group (acyl prodrugs) or a carbamate group (carbamate prodrugs). More detailed information relating to prodrugs is found, for example, in Fleisher et al., Advanced Drug Delivery Reviews 19 (1996) 115; Design of Prodrugs, H. Bundgaard (ed.), Elsevier, 1985; or H. Bundgaard, Drugs of the Future 16 (1991) 443. The term “derivative” is also used to describe all solvates, for example hydrates or adducts (e.g., adducts with alcohols), active metabolites, and salts of the parent compound. The type of salt that may be prepared depends on the nature of the moieties within the compound. For example, acidic groups, for example carboxylic acid groups, can form, for example, alkali metal salts or alkaline earth metal salts (e.g., sodium salts, potassium salts, magnesium salts and calcium salts, and also salts with physiologically tolerable quaternary ammonium ions and acid addition salts with ammonia and physiologically tolerable organic amines such as, for example, triethylamine, ethanolamine or tris-(2-hydroxyethyl)amine). Basic groups can form acid addition salts, for example with inorganic acids such as hydrochloric acid, sulfuric acid or phosphoric acid, or with organic carboxylic acids and sulfonic acids such as acetic acid, citric acid, benzoic acid, maleic acid, fumaric acid, tartaric acid, methanesulfonic acid or p-toluenesulfonic acid. Compounds which simultaneously contain a basic group and an acidic group, for example a carboxyl group in addition to basic nitrogen atoms, can be present as zwitterions. Salts can be obtained by customary methods known to those skilled in the art, for example by combining a compound with an inorganic or organic acid or base in a solvent or diluent, or from other salts by cation exchange or anion exchange.

As discussed above, the present invention provides compositions, methods and devices relating to medical implants, which greatly increase the ability to inhibit the formation of reactive scar tissue on, or around, the surface of the device or implant. Described in more detail below are methods for constructing medical implants, compositions and methods for generating medical implants which inhibit fibrosis, and methods for utilizing such medical implants.

A. Medical Implants

In one aspect, medical implants of the present invention are coated with, or otherwise adapted to release an agent which inhibits the formation of scar tissue. Representative examples of medical implants include: vascular stents, gastrointestinal stents, tracheal/bronchial stents, genital-urinary stents, ENT stents, intraocular lenses, implants for hypertrophic scars and keloids, vascular grafts, anastomotic connector devices, pacemaker leads, CVCs, films and meshes, ventricular assists devices, spinal prostheses, surgical adhesion barriers, glaucoma drainage devices, prosthetic heart valves, tympanostomy tubes, penile implants, endotracheal and tracheostomy tubes, peritoneal dialysis catheters, intracranial pressure monitors, vena cava filters, and gastrointestinal drainage tubes.

B. Therapeutic Agents

Suitable fibrosis or stenosis-inhibiting agents may be readily determined based upon the in vitro and in vivo (animal) models such as those provided in Examples 26-36. The assay set forth in Example 29 may be used to determine whether an agent is able to inhibit cell proliferation in fibroblasts and/or smooth muscle cells. In one aspect of the invention, the agent has an IC₅₀ for inhibition of cell proliferation within a range of about 10⁻⁶ to about 10⁻¹⁰ M. The assay set forth in Example 33 may be used to determine whether an agent may inhibit migration of fibroblasts and/or smooth muscle cells. In one aspect of the invention, the agent has an IC₅₀ for inhibition of cell migration within a range of about 10⁻⁶ to about 10⁻⁹M. Assays set forth herein may be used to determine whether an agent is able to inhibit inflammatory processes, including nitric oxide production in macrophages (Example 26), and/or TNF-alpha production by macrophages (Example 27), and/or IL-1 beta production by macrophages (Example 34), and/or IL-8 production by macrophages (Example 35), and/or inhibition of MCP-1 by macrophages (Example 36). In one aspect of the invention, the agent has an IC₅₀ for inhibition of any one of these inflammatory processes within a range of about 10⁻⁶ to about 10⁻¹⁰M. The assay set forth in Example 31 may be used to determine whether an agent is able to inhibit MMP production. In one aspect of the invention, the agent has an IC₅₀ for inhibition of MMP production within a range of about 10⁻⁴ to about 10⁻⁸M. The assay set forth in Example 39 (also known as the CAM assay) may be used to determine whether an agent is able to inhibit angiogenesis. In one aspect of the invention, the agent has an IC₅₀ for inhibition of angiogenesis within a range of about 10⁻⁶ to about 10⁻¹⁰M. Agents which inhibit fibrosis can also be identified through in vivo models including inhibition of intimal hyperplasia development in the rat balloon carotid artery model (Example 30) and/or a reduction of surgical adhesions formation in rabbit surgical adhesions model (Example 28).

Numerous therapeutic compounds have been identified that are of utility in the invention including:

1) Angiogenesis Inhibitors

In one embodiment, the pharmacologically active compound is an angiogenesis inhibitor (e.g., 2-ME (NSC-659853), PI-88 (D-mannose, O-6-O-phosphono-alpha-D-mannopyranosyl-(1-3)-O-alpha-D-mannopyranosyl-(1-3)-O-alpha-D-mannopyranosyl-(1-3)-O-alpha-D-mannopyranosyl-(1-2)-hydrogen sulphate), thalidomide (1H-isoindole-1,3(2H)-dione, 2-(2,6-dioxo-3-piperidinyl)-), CDC-394, CC-5079, ENMD-0995 (S-3-amino-phthalidoglutarimide), AVE-8062A, vatalanib, SH-268, halofuginone hydrobromide, atiprimod dimaleate (2-azaspivo[4.5]decane-2-propanamine, N,N-diethyl-8,8-dipropyl, dimaleate), ATN-224, QHIR-258, combretastatin A-4 (phenol, 2-methoxy-5-[2-(3,4,5-trimethoxyphenyl)ethenyl]-, (Z)-), GCS-100LE, or an analogue or derivative thereof).

2) 5-Lipoxygenase Inhibitors and Antagonists

In another embodiment, the pharmacologically active compound is a 5-lipoxygenase inhibitor or antagonist (e.g., Wy-50295 (2-naphthaleneacetic acid, alpha-methyl-6-(2-quinolinylmethoxy)-, (S)-), ONO-LP-269 (2,11,14-eicosatrienamide, N-(4-hydroxy-2-(1H-tetrazol-5-yl)-8-quinolinyl)-, (E,Z,Z)-), licofelone (1H-pyrrolizine-5-acetic acid, 6-(4-chlorophenyl)-2,3-dihydro-2,2-dimethyl-7-phenyl-), CMI-568 (urea, N-butyl-N-hydroxy-N′-(4-(3-(methylsulfonyl)-2-propoxy-5-(tetrahydro-5-(3,4,5-trimethoxyphenyl)-2-fura nyl)phenoxy)butyl)-,trans-), IP-751 ((3R,4R)-(delta 6)-THC-DMH-11-oic acid), PF-5901 (benzenemethanol, alpha-pentyl-3-(2-quinolinylmethoxy)-), LY-293111 (benzoic acid, 2-(3-(3-((5-ethyl-4′-fluoro-2-hydroxy(1,1′-biphenyl)-4-yl)oxy)propoxy)-2-propylphenoxy)-), RG-5901-A (benzenemethanol, alpha-pentyl-3-(2-quinolinylmethoxy)-, hydrochloride), rilopirox (2(1H)-pyridinone, 6-((4-(4-chlorophenoxy)phenoxy)methyl)-1-hydroxy-4-methyl-), L-674636 (acetic acid, ((4-(4-chlorophenyl)-1-(4-(2-quinolinylmethoxy)phenyl)butyl)thio)-AS)), 7-((3-(4-methoxy-tetrahydro-2H-pyran-4-yl)phenyl)methoxy)-4-phenylnaphtho(2,3-c)furan-1 (3H)-one, MK-886 (1H-indole-2-propanoic acid, 1-((4-chlorophenyl)methyl)-3-((1,1-dimethylethyl)thio)-alpha, alpha-dimethyl-5-(1-methylethyl)-), quiflapon (1H-indole-2-propanoic acid, 1-((4-chlorophenyl)methyl)-3-((1,1-dimethylethyl)thio)-alpha, alpha-dimethyl-5-(2-quinolinylmethoxy)-), quiflapon (1H-indole-2-propanoic acid, 1-((4-chlorophenyl)methyl)-3-((1,1-dimethylethyl)thio)-alpha, alpha-dimethyl-5-(2-quinolinylmethoxy)-), docebenone (2,5-cyclohexadiene-1,4-dione, 2-(12-hydroxy-5,10-dodecadiynyl)-3,5,6-trimethyl-), zileuton (urea, N-(1-benzo(b)thien-2-ylethyl)-N-hydroxy-), or an analogue or derivative thereof).

3) Chemokine Receptor Antagonists CCR (1, 3, and 5)

In another embodiment, the pharmacologically active compound is a chemokine receptor antagonist which inhibits one or more subtypes of CCR (1, 3, and 5) (e.g., ONO-4128 (1,4,9-triazaspiro(5.5)undecane-2,5-dione, 1-butyl-3-(cyclohexylmethyl)-9-((2,3-dihydro-1,4-benzodioxin-6-yl)methyl-), L-381, CT-112 (L-arginine, L-threonyl-L-threonyl-L-seryl-L-glutaminyl-L-valyl-L-arginyl-L-prolyl-), AS-900004, SCH-C, ZK-811752, PD-172084, UK-427857, SB-380732, vMIP II, SB-265610, DPC-168, TAK-779 (N,N-dimethyl-N-(4-(2-(4-methylphenyl)-6,7-dihydro-5H-benzocyclohepten-8-ylcarboxamido)benyl)tetrahydro-2H-pyran-4-aminium chloride), TAK-220, KRH-1120), GSK766994, SSR-150106, or an analogue or derivative thereof). Other examples of chemokine receptor antagonists include a-Immuhokine-NNSO₃, BX-471, CCX-282, Sch-350634; Sch-351125; Sch-417690; SCH-C, and analogues and derivatives thereof.

4) Cell Cycle Inhibitors

In another embodiment, the pharmacologically active compound is a cell cycle inhibitor. Representative examples of such agents include taxanes (e.g., paclitaxel (discussed in more detail below) and docetaxel) (Schiff et al., Nature 277: 665-667,1979; Long and Fairchild, Cancer Research 54: 4355-4361, 1994; Ringel and Horwitz, J. Natl Cancer Inst. 83(4): 288-291, 1991; Pazdur et al., Cancer Treat. Rev. 19(40): 351-386, 1993), etanidazole, nimorazole (B. A. Chabner and D. L. Longo. Cancer Chemotherapy and Biotherapy—Principles and Practice. Lippincott-Raven Publishers, New York, 1996, p. 554), perfluorochemicals with hyperbaric oxygen, transfusion, erythropoietin, BW12C, nicotinamide, hydralazine, BSO, WR-2721, IudR, DUdR, etanidazole, WR-2721, BSO, mono-substituted keto-aldehyde compounds (L. G. Egyud. Keto-aldehyde-amine addition products and method of making same. U.S. Pat. No. 4,066,650, Jan. 3, 1978), nitroimidazole (K. C. Agrawal and M. Sakaguchi. Nitroimidazole radiosensitizers for Hypoxic tumor cells and compositions thereof. U.S. Pat. No. 4,462,992, Jul. 31, 1984), 5-substituted-4-nitroimidazoles (Adams et al., Int J. Radiat. Biol. Relat. Stud. Phys., Chem. Med. 40(2): 153-61, 1981), SR-2508 (Brown et al., Int J. Radiat Oncol., Biol. Phys. 7(6): 695-703, 1981), 2H-isoindolediones (J. A. Myers, 2H-Isoindolediones, the synthesis and use as radiosensitizers. U.S. Pat. No. 4,494,547, Jan. 22, 1985), chiral (((2-bromoethyl)-amino)methyl)-nitro-1H-imidazole-1-ethanol (V. G. Beylin, et al., Process for preparing chiral (((2-bromoethyl)-amino)methyl)-nitro-1H-imidazole-1-ethanol and related compounds. U.S. Pat. No. 5,543,527, Aug. 6, 1996; U.S. Pat. No. 4,797,397; Jan. 10, 1989; U.S. Pat. No. 5,342,959, Aug. 30, 1994), nitroaniline derivatives (W. A. Denny, et al. Nitroaniline derivatives and the use as anti-tumor agents. U.S. Pat. No. 5,571,845, Nov. 5, 1996), DNA-affinic hypoxia selective cytotoxins (M.V. Papadopoulou-Rosenzweig. DNA-affinic hypoxia selective cytotoxins. U.S. Pat. No. 5,602,142, Feb. 11, 1997), halogenated DNA ligand (R. F. Martin. Halogenated DNA ligand radiosensitizers for cancer therapy. U.S. Pat. No. 5,641,764, Jun. 24, 1997), 1,2,4 benzotriazine oxides (W. W. Lee et al. 1,2,4-benzotriazine oxides as radiosensitizers and selective cytotoxic agents. U.S. Pat. No. 5,616,584, Apr. 1, 1997; U.S. Pat. No. 5,624,925, Apr. 29, 1997; Process for Preparing 1,2,4 Benzotriazine oxides. U.S. Pat. No. 5,175,287, Dec. 29, 1992), nitric oxide (J. B. Mitchell et al., Use of Nitric oxide releasing compounds as hypoxic cell radiation sensitizers. U.S. Pat. No. 5,650,442, Jul. 22, 1997), 2-nitroimidazole derivatives (M. J. Suto et al. 2-Nitroimidazole derivatives useful as radiosensitizers for hypoxic tumor cells. U.S. Pat. No. 4,797,397, Jan. 10, 1989; T. Suzuki. 2-Nitroimidazole derivative, production thereof, and radiosensitizer containing the same as active ingredient. U.S. Pat. No. 5,270,330, Dec. 14, 1993; T. Suzuki et al. 2-Nitroimidazole derivative, production thereof, and radiosensitizer containing the same as active ingredient. U.S. Pat. No. 5,270,330, Dec. 14, 1993; T. Suzuki. 2-Nitroimidazole derivative, production thereof and radiosensitizer containing the same as active ingredient; Patent EP 0 513 351 B1, Jan. 24, 1991), fluorine-containing nitroazole derivatives (T. Kagiya. Fluorine-containing nitroazole derivatives and radiosensitizer comprising the same. U.S. Pat. No. 4,927,941, May 22, 1990), copper (M. J. Abrams. Copper Radiosensitizers. U.S. Pat. No. 5,100,885, Mar. 31, 1992), combination modality cancer therapy (D. H. Picker et al. Combination modality cancer therapy. U.S. Pat. No. 4,681,091, Jul. 21, 1987). 5-CldC or (d)H₄U or 5-halo-2′-halo-2′-deoxy-cytidine or -uridine derivatives (S. B. Greer. Method and Materials for sensitizing neoplastic tissue to radiation. U.S. Pat. No. 4,894,364 Jan. 16, 1990), platinum complexes (K. A. Skov. Platinum Complexes with one radiosensitizing ligand. U.S. Pat. No. 4,921,963. May 1, 1990; K. A. Skov. Platinum Complexes with one radiosensitizing ligand. Patent EP 0 287 317 A3), fluorine-containing nitroazole (T. Kagiya, et al. Fluorine-containing nitroazole derivatives and radiosensitizer comprising the same. U.S. Pat. No. 4,927,941. May 22, 1990), benzamide (W. W. Lee. Substituted Benzamide Radiosensitizers. U.S. Pat. No. 5,032,617, Jul. 16, 1991), autobiotics (L. G. Egyud. Autobiotics and the use in eliminating nonself cells in vivo. U.S. Pat. No. 5,147,652. Sep. 15, 1992), benzamide and nicotinamide (W. W. Lee et al. Benzamide and Nictoinamide Radiosensitizers. U.S. Pat. No. 5,215,738, Jun. 1, 1993), acridine-intercalator (M. Papadopoulou-Rosenzweig. Acridine Intercalator based hypoxia selective cytotoxins. U.S. Pat. No. 5,294,715, Mar. 15, 1994), fluorine-containing nitroimidazole (T. Kagiya et al. Fluorine containing nitroimidazole compounds. U.S. Pat. No. 5,304,654, Apr. 19, 1994), hydroxylated texaphyrins (J. L. Sessler et al. Hydroxylated texaphrins. U.S. Pat. No. 5,457,183, Oct. 10, 1995), hydroxylated compound derivative (T. Suzuki et al. Heterocyclic compound derivative, production thereof and radiosensitizer and antiviral agent containing said derivative as active ingredient. Publication Number 011106775 A (Japan), Oct. 22, 1987; T. Suzuki et al. Heterocyclic compound derivative, production thereof and radiosensitizer, antiviral agent and anti cancer agent containing said derivative as active ingredient. Publication Number 01139596 A (Japan), Nov. 25, 1987; S. Sakaguchi et al. Heterocyclic compound derivative, its production and radiosensitizer containing said derivative as active ingredient; Publication Number 63170375 A (Japan), Jan. 7, 1987), fluorine containing 3-nitro-1,2,4-triazole (T. Kagitani et al. Novel fluorine-containing 3-nitro-1,2,4-triazole and radiosensitizer containing same compound. Publication Number 02076861 A (Japan), Mar. 31, 1988), 5-thiotretrazole derivative or its salt (E. Kano et al. Radiosensitizer for Hypoxic cell. Publication Number 61010511 A (Japan), Jun. 26, 1984), Nitrothiazole (T. Kagitani et al. Radiation-sensitizing agent. Publication Number 61167616 A (Japan) Jan. 22, 1985), imidazole derivatives (S. Inayma et al. Imidazole derivative. Publication Number 6203767 A (Japan) Aug. 1, 1985; Publication Number 62030768 A (Japan) Aug. 1, 1985; Publication Number 62030777 A (Japan) Aug. 1, 1985), 4-nitro-1,2,3-triazole (T. Kagitani et al. Radiosensitizer. Publication Number 62039525 A (Japan), Aug. 15, 1985), 3-nitro-1,2,4-triazole (T. Kagitani et al. Radiosensitizer. Publication Number 62138427 A (Japan), Dec. 12, 1985), Carcinostatic action regulator (H. Amagase. Carcinostatic action regulator. Publication Number 63099017 A (Japan), Nov. 21, 1986), 4,5-dinitroimidazole derivative (S. Inayama. 4,5-Dinitroimidazole derivative. Publication Number 63310873 A (Japan) Jun. 9, 1987), nitrotriazole Compound (T. Kagitanil Nitrotriazole Compound. Publication Number 07149737 A (Japan) Jun. 22, 1993), cisplatin, doxorubin, misonidazole, mitomycin, tiripazamine, nitrosourea, mercaptopurine, methotrexate, flurouracil, bleomycin, vincristine, carboplatin, epirubicin, doxorubicin, cyclophosphamide, vindesine, etoposide (I. F. Tannock. Review Article: Treatment of Cancer with Radiation and Drugs. Journal of Clinical Oncology 14(12): 3156-3174, 1996), camptothecin (Ewend M. G. et al. Local delivery of chemotherapy and concurrent external beam radiotherapy prolongs survival in metastatic brain tumor models. Cancer Research 56(22): 5217-5223, 1996) and paclitaxel (Tishler R. B. et al. Taxol: a novel radiation sensitizer. International Journal of Radiation Oncology and Biological Physics 22(3): 613-617, 1992).

A number of the above-mentioned cell cycle inhibitors also have a wide variety of analogues and derivatives, including, but not limited to, cisplatin, cyclophosphamide, misonidazole, tiripazamine, nitrosourea, mercaptopurine, methotrexate, flurouracil, epirubicin, doxorubicin, vindesine and etoposide. Analogues and derivatives include (CPA)₂Pt(DOLYM) and (DACH)Pt(DOLYM) cisplatin (Choi et al., Arch. Pharmacal Res. 22(2): 151-156, 1999), Cis-(PtCl₂(4,7-H-5-methyl-7-oxo)1,2,4(triazolo(1,5-a)pyrimidine)₂) (Navarro et al., J. Med. Chem. 41(3): 332-338, 1998), (Pt(cis-1,4-DACH)(trans-Cl₂)(CBDCA)).½MeOH cisplatin (Shamsuddin et al., Inorg. Chem. 36(25): 5969-5971, 1997), 4-pyridoxate diammine hydroxy platinum (Tokunaga et al., Pharm. Sci. 3(7): 353-356, 1997), Pt(II) . . . Pt(II) (Pt₂(NHCHN(C(CH₂)(CH₃)))₄) (Navarro et al., Inorg. Chem. 35(26): 7829-7835, 1996), 254-S cisplatin analogue (Koga et al., Neurol. Res. 18(3): 244-247, 1996), o-phenylenediamine ligand bearing cisplatin analogues (Koeckerbauer & Bednarski, J. Inorg. Biochem. 62(4): 281-298, 1996), trans,cis-(Pt(OAc)₂I₂(en)) (Kratochwil et al., J. Med. Chem. 39(13): 2499-2507, 1996), estrogenic 1,2-diarylethylenediamine ligand (with sulfur-containing amino acids and glutathione) bearing cisplatin analogues (Bednarski, J. Inorg. Biochem. 62(1): 75, 1996), cis-1,4-diaminocyclohexane cisplatin analogues (Shamsuddin et al., J. Inorg. Biochem. 61(4): 291-301, 1996), 5′ orientational isomer of cis-(Pt(NH₃)(4-aminoTEMP-O){d(GpG)}) (Dunham & Lippard, J. Am. Chem. Soc. 117(43): 10702-12, 1995), chelating diamine-bearing cisplatin analogues (Koeckerbauer & Bednarski, J. Pharm. Sci. 84(7): 819-23, 1995), 1,2-diarylethyleneamine ligand-bearing cisplatin analogues (Otto et al., J. Cancer Res. Clin. Oncol. 121(1): 31-8, 1995), (ethylenediamine)platinum(II) complexes (Pasini et al., J. Chem. Soc., Dalton Trans. 4: 579-85, 1995), CI-973 cisplatin analogue (Yang et al., Int J. Oncol. 5(3): 597-602, 1994), cis-diamminedichloroplatinum(II) and its analogues cis-1,1-cyclobutanedicarbosylato(2R)-2-methyl-1,4-butanediammineplatinum(II) and cis-diammine(glycolato)platinum (Claycamp & Zimbrick, J. Inorg. Biochem., 26(4): 257-67, 1986; Fan et al., Cancer Res. 48(11): 3135-9, 1988; Heiger-Bernays et al., Biochemistry 29(36): 8461-6, 1990; Kikkawa et al., J. Exp. Clin. Cancer Res. 12(4): 233-40, 1993; Murray et al., Biochemistry 31(47): 11812-17, 1992; Takahashi et al., Cancer Chemother. Pharmacol. 33(1): 31-5, 1993), cis-amine-cyclohexylamine-dichloroplatinum(II) (Yoshida et al., Biochem. Pharmacol. 48(4): 793-9, 1994), gem-diphosphonate cisplatin analogues (FR 2683529), (meso-1,2-bis(2,6-dichloro-4-hydroxyplenyl)ethylenediamine) dichloroplatinum(II) (Bednarski et al., J. Med. Chem. 35(23): 4479-85, 1992), cisplatin analogues containing a tethered dansyl group (Hartwig et al., J. Am. Chem. Soc. 114(21): 8292-3, 1992), platinum(II) polyamines (Siegmann et al., Inorg. Met-Containing Polym. Mater., (Proc. Am. Chem. Soc. Int. Symp.), 335-61, 1990), cis-(3H)dichloro(ethylenediamine)platinum(II) (Eastman, Anal. Biochem. 197(2): 311-15, 1991), trans-diamminedichloroplatinum(II) and cis-(Pt(NH₃)₂(N₃-cytosine)Cl) (Bellon & Lippard, Biophys. Chem. 35(2-3): 179-88, 1990), 3H-cis-1,2-diaminocyclohexanedichloroplatinum(II) and 3H-cis-1,2-diaminocyclohexane-malonatoplatinum (II) (Oswald et al., Res. Commun. Chem. Pathol. Pharmacol. 64(1): 41-58, 1989), diaminocarboxylatoplatinum (EPA 296321), trans-(D,1)-1,2-diaminocyclohexane carrier ligand-bearing platinum analogues (Wyrick & Chaney, J. Labelled Compd. Radiopharm. 25(4): 349-57, 1988), aminoalkylaminoanthraquinone-derived cisplatin analogues (Kitov et al., Eur. J. Med. Chem. 23(4): 381-3, 1988), spiroplatin, carboplatin, iproplatin and JM40 platinum analogues (Schroyen et al., Eur. J. Cancer Clin. Oncol. 24(8): 1309-12, 1988), bidentate tertiary diamine-containing cisplatinum derivatives (Orbell et al., Inorg. Chim. Acta 152(2): 125-34, 1988), platinum(II), platinum(IV) (Liu & Wang, Shandong Yike Daxue Xuebao 24(1): 35-41, 1986), cis-diammine(1,1-cyclobutanedicarboxylato-)platinum(II) (carboplatin, JM8) and ethylenediammine-malonatoplatinum(II) (JM40) (Begg et al., Radiother. Oncol. 9(2): 157-65, 1987), JM8 and JM9 cisplatin analogues (Harstrick et al., Int. J. Androl. 10(1); 139-45, 1987), (NPr4)₂((PtCl4).cis-(PtCl2-(NH2Me)₂)) (Brammer et al., J. Chem. Soc., Chem. Commun. 6: 443-5,1987), aliphatic tricarboxylic acid platinum complexes (EPA 185225), cis-dichloro(amino acid)(tert-butylamine)platinum(II) complexes (Pasini & Bersanetti, Inorg. Chim. Acta 107(4): 259-67, 1985); 4-hydroperoxycylcophosphamide (Ballard et al., Cancer Chemother. Pharmacol. 26(6): 397-402, 1990), acyclouridine cyclophosphamide derivatives (Zakerinia et al., Helv. Chim. Acta 73(4): 912-15, 1990), 1,3,2-dioxa- and -oxazaphosphorinane cyclophosphamide analogues (Yang et al., Tetrahedron 44(20): 6305-14, 1988), C5-substituted cyclophosphamide analogues (Spada, University of Rhode Island Dissertation, 1987), tetrahydrooxazine cyclophosphamide analogues (Valente, University of Rochester Dissertation, 1988), phenyl ketone cyclophosphamide analogues (Hales et al., Teratology 39(1): 31-7, 1989), phenylketophosphamide cyclophosphamide analogues (Ludeman et al., J. Med. Chem. 29(5): 716-27, 1986), ASTA Z-7557 cyclophosphamide analogues (Evans et al., Int. J. Cancer 34(6): 883-90, 1984), 3-(1-oxy-2,2,6,6-tetramethyl-4-piperidinyl)cyclophosphamide (Tsui et al., J. Med. Chem. 25(9): 1106-10, 1982), 2-oxobis(2-β-chloroethylamino)-4-,6-dimethyl-1,3,2-oxazaphosphorinane cyclophosphamide (Carpenter et al., Phosphorus Sulfur 12(3): 287-93, 1982), 5-fluoro- and 5-chlorocyclophosphamide (Foster et al., J. Med. Chem. 24(12): 1399-403,1981), cis- and trans-4-phenylcyclophosphamide (Boyd et al., J. Med. Chem. 23(4): 372-5, 1980), 5-bromocyclophosphamide, 3,5-dehydrocyclophosphamide (Ludeman et al., J. Med. Chem. 22(2): 151-8, 1979), 4-ethoxycarbonyl cyclophosphamide analogues (Foster, J. Pharm. Sci. 67(5): 709-10, 1978), arylaminotetrahydro-2H-1,3,2-oxazaphosphorine 2-oxide cyclophosphamide analogues (Hamacher, Arch. Pharm. (Weinheim, Ger.) 310(5): J, 428-34, 1977), NSC-26271 cyclophosphamide analogues (Montgomery & Struck, Cancer Treat. Rep. 60(4): J381-93, 1976), benzo annulated cyclophosphamide analogues (Ludeman & Zon, J. Med. Chem. 18(12): J1251-3, 1975), 6-trifluoromethylcyclophosphamide (Farmer & Cox, J. Med. Chem. 18(11): J1106-10, 1975), 4-methylcyclophosphamide and 6-methycyclophosphamide analogues (Cox et al., Biochem. Pharmacol. 24(5): J599-606,1975); FCE 23762 doxorubicin derivative (Quaglia et al., J. Liq. Chromatogr. 17(18): 3911-3923, 1994), annamycin (Zou et al., J. Pharm. Sci. 82(11): 1151-1154, 1993), ruboxyl (Rapoport et al., J. Controlled Release 58(2): 153-162, 1999), anthracycline disaccharide doxorubicin analogue (Pratesi et al., Clin. Cancer Res. 4(11): 2833-2839, 1998), N-(trifluoroacetyl)doxorubicin and 4′-O-acetyl-N-(trifluoroacetyl)doxorubicin (Berube & Lepage, Synth. Commun. 28(6): 1109-1116, 1998), 2-pyrrolinodoxorubicin (Nagy et al., Proc. Nat'l Acad. Sci. U.S.A. 95(4): 1794-1799, 1998), disaccharide doxorubicin analogues (Arcamone et al., J. Nat'l Cancer Inst. 89(16): 1217-1223, 1997), 4-demethoxy-7-O-(2,6-dideoxy-4-O-(2,3,6-trideoxy-3-amino-α-L-lyxo-hexopyranosyl)-α-L-lyxo-hexopyranosyl)-adriamicinone doxorubicin disaccharide analogue (Monteagudo et al., Carbohydr. Res. 300(1): 11-16, 1997), 2-pyrrolinodoxorubicin (Nagy et al., Proc. Nat'l Acad. Sci. U.S.A. 94(2): 652-656, 1997), morpholinyl doxorubicin analogues (Duran et al., Cancer Chemother. Pharmacol. 38(3): 210-216, 1996), enaminomalonyl-α-alanine doxorubicin derivatives (Seitz et al., Tetrahedron Lett. 36(9): 1413-16, 1995), cephalosporin doxorubicin derivatives (Vrudhula et al., J. Med. Chem. 38(8): 1380-5, 1995), hydroxyrubicin (Solary et al., Int J. Cancer 58(1): 85-94, 1994), methoxymorpholino doxorubicin derivative (Kuhl et al., Cancer Chemother. Pharmacol. 33(1): 10-16, 1993), (6-maleimidocaproyl)hydrazone doxorubicin derivative (Willner et al., Bioconjugate Chem. 4(6): 521-7, 1993), N-(5,5-diacetoxypent-1-yl) doxorubicin (Cherif & Farquhar, J. Med. Chem. 35(17): 3208-14, 1992), FCE 23762 methoxymorpholinyl doxorubicin derivative (Ripamonti et al., Br. J. Cancer 65(5): 703-7, 1992), N-hydroxysuccinimide ester doxorubicin derivatives (Demant et al., Biochim. Biophys. Acta 1118(1): 83-90, 1991), polydeoxynucleotide doxorubicin derivatives (Ruggiero et al., Biochim. Biophys. Acta 1129(3): 294-302, 1991), morpholinyl doxorubicin derivatives (EPA 434960), mitoxantrone doxorubicin analogue (Krapcho et al., J. Med. Chem. 34(8): 2373-80. 1991), AD198 doxorubicin analogue (Traganos et al., Cancer Res. 51(14): 3682-9, 1991), 4-demethoxy-3′-N-trifluoroacetyldoxorubicin (Horton et al., Drug Des. Delivery 6(2): 123-9, 1990), 4′-epidoxorubicin (Drzewoski et al., Pol. J. Pharmacol. Pharm. 40(2): 159-65,1988; Weenen et al., Eur. J. Cancer Clin. Oncol. 20(7): 919-26,1984), alkylating cyanomorpholino doxorubicin derivative (Scudder et al., J. Nat'l Cancer Inst. 80(16): 1294-8, 1988), deoxydihydroiodooxorubicin (EPA 275966), adriblastin (Kalishevskaya et al., Vestn. Mosk. Univ., 16(Biol. 1): 21-7, 1988), 4′-deoxydoxorubicin (Schoeizel et al., Leuk. Res. 10(12): 1455-9, 1986), 4-demethyoxy-4′-o-methyldoxorubicin (GIuliani et al., Proc. Int. Congr. Chemother. 16: 285-70-285-77,1983), 3′-deamino-3′-hydroxydoxorubicin (Horton et al., J. Antibiot. 37(8): 853-8, 1984), 4-demethyoxy doxorubicin analogues (Barbieri et al., Drugs Exp. Clin. Res. 10(2): 85-90, 1984), N-L-leucyl doxorubicin derivatives (Trouet et al., Anthracyclines (Proc. Int. Symp. Tumor Pharmacother.), 179-81, 1983), 3′-deamino-3′-(4-methoxy-1-piperidinyl) doxorubicin derivatives (U.S. Pat. No. 4,314,054), 3′-deamino-3′-(4-mortholinyl) doxorubicin derivatives (U.S. Pat. No. 4,301,277), 4′-deoxydoxorubicin and 4′-o-methyldoxorubicin (GIuliani et al., Int. J. Cancer 27(1): 5-13, 1981), aglycone doxorubicin derivatives (Chan & Watson, J. Pharm. Sci. 67(12): 1748-52, 1978), SM 5887 (Pharma Japan 1468: 20, 1995), MX-2 (Pharma Japan 1420: 19, 1994), 4′-deoxy-13(S)-dihydro-4′-iododoxorubicin (EP 275966), morpholinyl doxorubicin derivatives (EPA 434960), 3′-deamino-3′-(4-methoxy-1-piperidinyl) doxorubicin derivatives (U.S. Pat. No. 4,314,054), doxorubicin-14-valerate, morpholinodoxorubicin (U.S. Pat. No. 5,004,606), 3′-deamino-3′-(3″-cyano-4″-morpholinyl doxorubicin; 3′-deamino-3′-(3″-cyano-4″-morpholinyl)-13-dihydoxorubicin; (3′-deamino-3′-(3″-cyano-4″-morpholinyl) daunorubicin; 3′-deamino-3′-(3″-cyano-4″-morpholinyl)-3-dihydrodaunorubicin; and 3′-deamino-3′-(4″-morpholinyl-5-iminodoxorubicin and derivatives (U.S. Pat. No. 4,585,859), 3′-deamino-3′-(4-methoxy-1-piperidinyl) doxorubicin derivatives (U.S. Pat. No. 4,314,054) and 3-deamino-3-(4-morpholinyl) doxorubicin derivatives (U.S. Pat. No. 4,301,277); 4,5-dimethylmisonidazole (Born et al., Biochem. Pharmacol. 43(6): 1337-44, 1992), azo and azoxy misonidazole derivatives (Gattavecchia & Tonelli, Int. J. Radiat. Biol. Relat. Stud. Phys., Chem. Med. 45(5): 469-77,1984); RB90740 (Wardman et al., Br. J. Cancer, 74 Suppl. (27): S70-S74, 1996); 6-bromo and 6-chloro-2,3-dihydro-1,4-benzothiazines nitrosourea derivatives (Rai et al., Heterocycl. Commun. 2(6): 587-592, 1996), diamino acid nitrosourea derivatives (Dulude et al., Bioorg. Med. Chem. Lett. 4(22): 2697-700, 1994; Dulude et al., Bioorg. Med. Chem. 3(2): 151-60, 1995), amino acid nitrosourea derivatives (Zheleva et al., Pharmazie 50(1): 25-6, 1995), 3′,4′-didemethoxy-3′,4′-dioxo-4-deoxypodophyllotoxin nitrosourea derivatives (Miyahara et al., Heterocycles 39(1): 361-9, 1994), ACNU (Matsunaga et al., Immunopharmacology 23(3): 199-204, 1992), tertiary phosphine oxide nitrosourea derivatives (Guguva et al., Pharmazie 46(8): 603, 1991), sulfamerizine and sulfamethizole nitrosourea derivatives (Chiang et al., Zhonghua Yaozue Zazhi 43(5): 401-6, 1991), thymidine nitrosourea analogues (Zhang et al., Cancer Commun. 3(4): 119-26, 1991), 1,3-bis(2-chloroethyl)-1-nitrosourea (August et al., Cancer Res. 51(6): 1586-90, 1991), 2,2,6,6-tetramethyl-1-oxopiperidiunium nitrosourea derivatives (U.S.S.R. 1261253), 2- and 4-deoxy sugar nitrosourea derivatives (U.S. Pat. No. 4,902,791), nitroxyl nitrosourea derivatives (U.S.S.R. 1336489), fotemustine (Boutin et al., Eur. J. Cancer Clin. Oncol. 25(9): 1311-16, 1989), pyrimidine (II) nitrosourea derivatives (Wei et al., Chung-hua Yao Hsuch Tsa Chih 41(1): 19-26,1989), CGP 6809 (Schieweck et al., Cancer Chemother. Pharmacol. 23(6): 341-7, 1989), B-3839 (Prajda et al., In Vivo 2(2): 151-4, 1988), 5-halogenocytosine nitrosourea derivatives (Chiang & Tseng, T'ai-wan Yao Hsuch Tsa Chih 38(1): 37-43, 1986), 1-(2-chloroethyl)-3-isobutyl-3-(β-maltosyl)-1-nitrosourea (Fujimoto & Ogawa, J. Pharmacobio-Dyn. 10(7): 341-5, 1987), sulfur-containing nitrosoureas (Tang et al., Yaoxue Xuebao 21(7): 502-9, 1986), sucrose, 6-((((2-chloroethyl)nitrosoamino-)carbonyl)amino)-6-deoxysucrose (NS-1C) and 6′-((((2-chloroethyl)nitrosoamino)carbonyl)amino)-6′-deoxysucrose (NS-1D) nitrosourea derivatives (Tanoh et al., Chemotherapy (Tokyo) 33(11): 969-77,1985), CNCC, RFCNU and chlorozotocin (Mena et al., Chemotherapy (Basel) 32(2): 131-7, 1986), CNUA (Edanami et al., Chemotherapy (Tokyo) 33(5): 455-61, 1985), 1-(2-chloroethyl)-3-isobutyl-3-(β-maltosyl)-1-nitrosourea (Fujimoto & Ogawa, Jpn. J. Cancer Res. (Gann) 76(7): 651-6,1985), choline-like nitrosoalkylureas (Belyaev et al., Izv. Akad. NAUK SSSR, Ser. Khim. 3: 553-7, 1985), sucrose nitrosourea derivatives (JP 84219300), sulfa drug nitrosourea analogues (Chiang et al., Proc. Natl. Sci. Counc., Repub. China, Part A 8(1): 18-22, 1984), DONU (Asanuma et al., J. Jpn. Soc. Cancer Ther. 17(8): 2035-43, 1982), N,N′-bis (N-(2-chloroethyl)-N-nitrosocarbamoyl)cystamine (CNCC) (Blazsek et al., Toxicol. Appl. Pharmacol. 74(2): 250-7, 1984), dimethylnitrosourea (Krutova et al., Izv. Akad. NAUK SSSR, Ser. Biol. 3: 439-45, 1984), GANU (Sava & Giraldi, Cancer Chemother. Pharmacol. 10(3): 167-9, 1983), CCNU (Capelli et al., Med., Biol., Environ. 11(1): 111-16, 1983), 5-aminomethyl-2′-deoxyuridine nitrosourea analogues (Shiau, Shih Ta Hsuch Pao (Taipei) 27: 681-9,1982), TA-077 (Fujimoto & Ogawa, Cancer Chemother. Pharmacol. 9(3): 134-9, 1982), gentianose nitrosourea derivatives (JP 82 80396), CNCC, RFCNU, RPCNU AND chlorozotocin (CZT) (Marzin et al., INSERM Symp., 19 (Nitrosoureas Cancer Treat.): 165-74,1981), thiocolchicine nitrosourea analogues (George, Shih Ta Hsuch Pao (Taipei) 25: 355-62, 1980), 2-chloroethyl-nitrosourea (Zeller & Eisenbrand, Oncology 38(1): 39-42, 1981), ACNU, (1-(4-amino-2-methyl-5-pyrimidinyl)methyl-3-(2-chloroethyl)-3-nitrosourea hydrochloride) (Shibuya et al., Gan To Kagaku Ryoho 7(8): 1393-401, 1980), N-deacetylmethyl thiocolchicine nitrosourea analogues (Lin et al., J. Med. Chem. 23(12): 1440-2, 1980), pyridine and piperidine nitrosourea derivatives (Crider et al., J. Med. Chem. 23(8): 848-51,1980), methyl-CCNU (Zimber & Perk, Refu. Vet 35(1): 28, 1978), phensuzimide nitrosourea derivatives (Crider et al., J. Med. Chem. 23(3): 324-6, 1980), ergoline nitrosourea derivatives (Crider et al., J. Med. Chem. 22(1): 32-5, 1979), glucopyranose nitrosourea derivatives (JP 78 95917), 1-(2-chloroethyl)-3-cyclohexyl-1-nitrosourea (Farmer et al., J. Med. Chem. 21(6): 514-20, 1978), 4-(3-(2-chloroethyl)-3-nitrosoureid-o)-cis-cyclohexanecarboxylic acid (Drewinko et al., Cancer Treat. Rep. 61(8): J1513-18, 1977), RPCNU (ICIG 1163) (Lamicol et al., Biomedicine 26(3): J176-81,1977), IOB-252 (Sorodoc et al., Rev. Roum. Med., Virol. 28(1): J 55-61, 1977), 1,3-bis(2-chloroethyl)-1-nitrosourea (BCNU) (Siebert & Eisenbrand, Mutat. Res. 42(1): J45-50, 1977), 1-tetrahydroxycyclopentyl-3-nitroso-3-(2-chloroethyl)-urea (U.S. Pat. No. 4,039,578), d-1-1-(β-chloroethyl)-3-(2-oxo-3-hexahydroazepinyl)-1-nitrosourea (U.S. Pat. No. 3,859,277) and gentianose nitrosourea derivatives (JP 57080396); 6-S-aminoacyloxymethyl mercaptopurine derivatives (Harada et al., Chem. Pharm. Bull. 43(10): 793-6, 1995), 6-mercaptopurine (6-MP) (Kashida et al., Biol. Pharm. Bull. 18(11): 1492-7, 1995), 7,8-polymethyleneimidazo-1,3,2-diazaphosphorines (Nilov et al., Mendeleev Commun. 2: 67, 1995), azathioprine (Chifotides et al., J. Inorg. Biochem. 56(4): 249-64, 1994), methyl-D-glucopyranoside mercaptopurine derivatives (Da Silva et al., Eur. J. Med. Chem. 29(2): 149-52, 1994) and s-alkynyl mercaptopurine derivatives (Ratsino et al., Khim.-Farm. Zh. 15(8): 65-7, 1981); indoline ring and a modified ornithine or glutamic acid-bearing methotrexate derivatives (Matsuoka et al., Chem. Pharm. Bull. 45(7): 1146-1150, 1997), alkyl-substituted benzene ring C bearing methotrexate derivatives (Matsuoka et al., Chem. Pharm. Bull. 44(12): 2287-2293, 1996), benzoxazine or benzothiazine moiety-bearing methotrexate derivatives (Matsuoka et al., J. Med. Chem. 40(1): 105-111, 1997), 10-deazaminopterin analogues (DeGraw et al., J. Med. Chem. 40(3): 370-376, 1997), 5-deazaminopterin and 5,10-dideazaminopterin methotrexate analogues (Piper et al., J. Med. Chem. 40(3): 377-384, 1997), indoline moiety-bearing methotrexate derivatives (Matsuoka et al., Chem. Pharm. Bull. 44(7): 1332-1337, 1996), lipophilic amide methotrexate derivatives (Pignatello et al., World Meet. Pharm., Biopharm. Pharm. Technol., 563-4, 1995), L-threo-(2S,4S)-4-fluoroglutamic acid and DL-3,3-difluoroglutamic acid-containing methotrexate analogues (Hart et al., J. Med. Chem. 39(1): 56-65, 1996), methotrexate tetrahydroquinazoline analogue (Gangjee, et al., J. Heterocycl. Chem. 32(1): 243-8, 1995), N-(α-aminoacyl)methotrexate derivatives (Cheung et al., Pteridines 3(1-2): 101-2, 1992), biotin methotrexate derivatives (Fan et al., Pteridines 3(1-2): 131-2, 1992), D-glutamic acid or D-erythrou, threo-4-fluoroglutamic acid methotrexate analogues (McGuire et al., Biochem. Pharmacol. 42(12): 2400-3, 1991), β,γ-methano methotrexate analogues (Rosowsky et al., Pteridines 2(3): 133-9, 1991), 10-deazaminopterin (10-EDAM) analogue (Braakhuis et al., Chem. Biol. Pteridines, Proc. Int. Symp. Pteridines Folic Acid Deriv., 1027-30,1989), γ-tetrazole methotrexate analogue (Kalman et al., Chem. Biol. Pteridines, Proc. Int. Symp. Pteridines Folic Acid Deriv., 1154-7, 1989), N-(L-α-aminoacyl)methotrexate derivatives (Cheung et al., Heterocycles 28(2): 751-8, 1989), meta and ortho isomers of aminopterin (Rosowsky et al., J. Med. Chem. 32(12): 2582, 1989), hydroxymethylmethotrexate (DE 267495), γ-fluoromethotrexate (McGuire et al., Cancer Res. 49(16): 4517-25, 1989), polyglutamyl methotrexate derivatives (Kumar et al., Cancer Res. 46(10): 5020-3, 1986), gem-diphosphonate methotrexate analogues (WO 88/06158), α- and γ-substituted methotrexate analogues (Tsushima et al., Tetrahedron 44(17): 5375-87, 1988), 5-methyl-5-deaza methotrexate analogues (U.S. Pat. No. 4,725,687), Nδ-acyl-Nα-(4-amino-4-deoxypteroyl)-L-ornithine derivatives (Rosowsky et al., J. Med. Chem. 31(7): 1332-7, 1988), 8-deaza methotrexate analogues (Kuehl et al., Cancer Res. 48(6): 1481-8, 1988), acivicin methotrexate analogue (Rosowsky et al., J. Med. Chem. 30(8): 1463-9, 1987), polymeric platinol methotrexate derivative (Carraher et al., Polym. Sci. Technol. (Plenum), 35(Adv. Biomed. Polym.): 311-24, 1987), methotrexate-γ-dimyristoylphophatidylethanolamine (Kinsky et al., Biochim. Biophys. Acta 917(2): 211-18,1987), methotrexate polyglutamate analogues (Rosowsky et al., Chem. Biol. Pteridines, Pteridines Folid Acid Deriv., Proc. Int. Symp. Pteridines Folid Acid Deriv.: Chem., Biol. Clin. Aspects: 985-8,1986), poly-γ-glutamyl methotrexate derivatives (Kisliuk et al., Chem. Biol. Pteridines, Pteridines Folid Acid Deriv., Proc. Int. Symp. Pteridines Folid Acid Deriv.: Chem., Biol. Clin. Aspects: 989-92, 1986), deoxyuridylate methotrexate derivatives (Webber et al., Chem. Biol. Pteridines, Pteridines Folid Acid Deriv., Proc. Int. Symp. Pteridines Folid Acid Deriv.: Chem., Biol. Clin. Aspects: 659-62, 1986), iodoacetyl lysine methotrexate analogue (Delcamp et al., Chem. Biol. Pteridines, Pteridines Folid Acid Deriv., Proc. Int. Symp. Pteridines Folid Acid Deriv.: Chem., Biol. Clin. Aspects: 807-9, 1986), 2,.omega.-diaminoalkanoid acid-containing methotrexate analogues (McGuire et al., Biochem. Pharmacol. 35(15): 2607-13, 1986), polyglutamate methotrexate derivatives (Kamen & Winick, Methods Enzymol. 122 (Vitam. Coenzymes, Pt. G): 339-46, 1986), 5-methyl-5-deaza analogues (Piper et al., J. Med. Chem. 29(6): 1080-7, 1986), quinazoline methotrexate analogue (Mastropaolo et al., J. Med. Chem. 29(1): 155-8, 1986), pyrazine methotrexate analogue (Lever & Vestal, J. Heterocycl. Chem. 22(1): 5-6, 1985), cysteic acid and homocysteic acid methotrexate analogues (U.S. Pat. No. 4,490,529), γ-tert-butyl methotrexate esters (Rosowsky et al., J. Med. Chem. 28(5): 660-7, 1985), fluorinated methotrexate analogues (Tsushima et al., Heterocycles 23(1): 45-9, 1985), folate methotrexate analogue (Trombe, J. Bacteriol. 160(3): 849-53, 1984), phosphonoglutamic acid analogues (Sturtz & Guillamot, Eur. J. Med. Chem.—Chim. Ther. 19(3): 267-73, 1984), poly(L-lysine)methotrexate conjugates (Rosowsky et al., J. Med. Chem. 27(7): 888-93,1984), dilysine and trilysine methotrexate derivates (Forsch & Rosowsky, J. Org. Chem. 49(7): 1305-9, 1984), 7-hydroxymethotrexate (Fabre et al., Cancer Res. 43(10): 4648-52, 1983), poly-γ-glutamyl methotrexate analogues (Piper & Montgomery, Adv. Exp. Med. Biol., 163 (Folyl Antifolyl Polyglutamates): 95-100, 1983), 3′,5′-dichloromethotrexate (Rosowsky & Yu, J. Med. Chem. 26(10): 1448-52, 1983), diazoketone and chloromethylketone methotrexate analogues (Gangjee et al., J. Pharm. Sci. 71(6): 717-19,1982), 10-propargylaminopterin and alkyl methotrexate homologs (Piper et al., J. Med. Chem. 25(7): 877-80, 1982), lectin derivatives of methotrexate (Lin et al., JNCI 66(3): 523-8, 1981), polyglutamate methotrexate derivatives (Galivan, Mol. Pharmacol. 17(1): 105-10, 1980), halogentated methotrexate derivatives (Fox, JNCI 58(4): J955-8, 1977), 8-alkyl-7,8-dihydro analogues (Chaykovsky et al., J. Med. Chem. 20(10): J1323-7, 1977), 7-methyl methotrexate derivatives and dichloromethotrexate (Rosowsky & Chen, J. Med. Chem. 17(12): J1308-11, 1974), lipophilic methotrexate derivatives and 3′,5′-dichloromethotrexate (Rosowsky, J. Med. Chem. 16(10): J1190-3, 1973), deaza amethopterin analogues (Montgomery et al., Ann. N.Y. Acad. Sci. 186: J227-34, 1971), MX068 (Pharma Japan, 1658:18, 1999) and cysteic acid and homocysteic acid methotrexate analogues (EPA 0142220); N3-alkylated analogues of 5-fluorouracil (Kozai et al., J. Chem. Soc., Perkin Trans. 1(19): 3145-3146, 1998), 5-fluorouracil derivatives with 1,4-oxaheteroepane moieties (Gomez et al., Tetrahedron 54(43): 13295-13312, 1998), 5-fluorouracil and nucleoside analogues (Li, Anticancer Res. 17(1A): 21-27, 1997), cis- and trans-5-fluoro-5,6-dihydro-6-alkoxyuracil (Van der Wilt et al., Br. J. Cancer 68(4): 702-7, 1993), cyclopentane 5-fluorouracil analogues (Hronowski & Szarek, Can. J. Chem. 70(4): 1162-9, 1992), A-OT-fluorouracil (Zhang et al., Zongguo Yiyao Gongye Zazhi 20(11): 513-15, 1989), N4-trimethoxybenzoyl-5′-deoxy-5-fluorocytidine and 5′-deoxy-5-fluorouridine (Miwa et al., Chem. Pharm. Bull. 38(4): 998-1003, 1990), 1-hexylcarbamoyl-5-fluorouracil (Hoshi et al., J. Pharmacobio-Dun. 3(9): 478-81, 1980; Maehara et al., Chemotherapy (Basel) 34(6): 484-9,1988), B-3839 (Prajda et al., In Vivo 2(2): 151-4, 1988), uracil-1-(2-tetrahydrofuryl)-5-fluorouracil (Anai et al., Oncology 45(3): 144-7,1988), 1-(2′-deoxy-2′-fluoro-β-D-arabinofuranosyl)-5-fluorouracil (Suzuko et al., Mol. Pharmacol. 31(3): 301-6, 1987), doxifluridine (Matuura et al., Oyo Yakuri 29(5): 803-31,1985), 5′-deoxy-5-fluorouridine (Bollag & Hartmann, Eur. J. Cancer 16(4): 427-32, 1980), 1-acetyl-3-O-toluyl-5-fluorouracil (Okada, Hiroshima J. Med. Sci. 28(1): 49-66, 1979), 5-fluorouracil-m-formylbenzene-sulfonate (JP 55059173), N′-(2-furanidyl)-5-fluorouracil (JP 53149985) and 1-(2-tetrahydrofuryl)-5-fluorouracil (JP 52089680); 4′-epidoxorubicin (Lanius, Adv. Chemother. Gastrointest. Cancer, (Int. Symp.), 159-67,1984); N-substituted deacetylvinblastine amide (vindesine) sulfates (Conrad et al., J. Med. Chem. 22(4): 391-400, 1979); and Cu(II)-VP-16 (etoposide) complex (Tawa et al., Bioorg. Med. Chem. 6(7): 1003-1008, 1998), pyrrolecarboxamidino-bearing etoposide analogues (Ji et al., Bioorg. Med. Chem. Lett. 7(5): 607-612, 1997), 4β-amino etoposide analogues (Hu, University of North Carolina Dissertation, 1992), γ-lactone ring-modified arylamino etoposide analogues (Zhou et al., J. Med. Chem. 37(2): 287-92, 1994), N-glucosyl etoposide analogue (Allevi et al., Tetrahedron Lett. 34(45): 7313-16, 1993), etoposide A-ring analogues (Kadow et al., Bioorg. Med. Chem. Lett. 2(1): 17-22, 1992), 4′-deshydroxy-4′-methyl etoposide (Saulnier et al., Bioorg. Med. Chem. Lett. 2(10): 1213-18, 1992), pendulum ring etoposide analogues (Sinha et al., Eur. J. Cancer 26(5): 590-3, 1990) and E-ring desoxy etoposide analogues (Saulnier et al., J. Med. Chem. 32(7): 1418-20, 1989).

Within one preferred embodiment of the invention, the cell cycle inhibitor is paclitaxel, a compound which disrupts mitosis (M-phase) by binding to tubulin to form abnormal mitotic spindles or an analogue or derivative thereof. Briefly, paclitaxel is a highly derivatized diterpenoid (Wani et al., J. Am. Chem. Soc. 93: 2325, 1971) which has been obtained from the harvested and dried bark of Taxus brevifolia (Pacific Yew) and Taxomyces Andreanae and Endophytic Fungus of the Pacific Yew (Stierle et al., Science 60: 214-216, 1993). “Paclitaxel” (which should be understood herein to include formulations, prodrugs, analogues and derivatives such as, for example, TAXOL (Bristol Myers Squibb, New York, N.Y., TAXOTERE (Aventis Pharmaceuticals, France), docetaxel, 10-desacetyl analogues of paclitaxel and 3′N-desbenzoyl-3′N-t-butoxy carbonyl analogues of paclitaxel) may be readily prepared utilizing techniques known to those skilled in the art (see, e.g., Schiff et al., Nature 277: 665-667,1979; Long and Fairchild, Cancer Research 54: 4355-4361, 1994; Ringel and Horwitz, J. Nat'l Cancer Inst. 83(4): 288-291, 1991; Pazdur et al., Cancer Treat. Rev. 19(4): 351-386, 1993; WO 94/07882; WO 94/07881; WO 94/07880; WO 94/07876; WO 93/23555; WO 93/10076; WO94/00156; WO 93/24476; EP 590267; WO 94/20089; U.S. Pat. Nos. 5,294,637; 5,283,253; 5,279,949; 5,274,137; 5,202,448; 5,200,534; 5,229,529; 5,254,580; 5,412,092; 5,395,850; 5,380,751; 5,350,866; 4,857,653; 5,272,171; 5,411,984; 5,248,796; 5,248,796; 5,422,364; 5,300,638; 5,294,637; 5,362,831; 5,440,056; 4,814,470; 5,278,324; 5,352,805; 5,411,984; 5,059,699; 4,942,184; Tetrahedron Letters 35(52): 9709-9712, 1994; J. Med. Chem. 35: 4230-4237, 1992; J. Med. Chem. 34: 992-998, 1991; J. Natural Prod. 57(10): 1404-1410, 1994; J. Natural Prod. 57(11): 1580-1583, 1994; J. Am. Chem. Soc. 110: 6558-6560, 1988), or obtained from a variety of commercial sources, including for example, Sigma Chemical Co., St. Louis, Mo. (T7402—from Taxus brevifolia).

Representative examples of paclitaxel derivatives or analogues include 7-deoxy-docetaxol, 7,8-cyclopropataxanes, N-substituted 2-azetidones, 6,7-epoxy paclitaxels, 6,7-modified paclitaxels, 10-desacetoxytaxol, 10-deacetyltaxol (from 10-deacetylbaccatin III), phosphonooxy and carbonate derivatives of taxol, taxol 2′,7-di(sodium 1,2-benzenedicarboxylate, 10-desacetoxy-11,12-dihydrotaxol-10,12(18)-diene derivatives, 10-desacetoxytaxol, Protaxol (2′- and/or 7-O-ester derivatives), (2′- and/or 7-O-carbonate derivatives), asymmetric synthesis of taxol side chain, fluoro taxols, 9-deoxotaxane, (13-acetyl-9-deoxobaccatine III, 9-deoxotaxol, 7-deoxy-9-deoxotaxol, 10-desacetoxy-7-deoxy-9-deoxotaxol, Derivatives containing hydrogen or acetyl group and a hydroxy and tert-butoxycarbonylamino, sulfonated 2′-acryloyltaxol and sulfonated 2′-O-acyl acid taxol derivatives, succinyltaxol, 2′-γ-aminobutyryltaxol formate, 2′-acetyl taxol, 7-acetyl taxol, 7-glycine carbamate taxol, 2′-OH-7-PEG(5000) carbamate taxol, 2′-benzoyl and 2′,7-dibenzoyl taxol derivatives, other prodrugs (2′-acetyltaxol; 2′,7-diacetyltaxol; 2′succinyltaxol; 2′-(beta-alanyl)-taxol); 2′gamma-aminobutyryltaxol formate; ethylene glycol derivatives of 2′-succinyltaxol; 2′-glutaryltaxol; 2′-(N,N-dimethylglycyl) taxol; 2′-(2-(N,N-dimethylamino)propionyl)taxol; 2′orthocarboxybenzoyl taxol; 2′aliphatic carboxylic acid derivatives of taxol, Prodrugs {2′(N,N-diethylaminopropionyl)taxol, 2′(N,N-dimethylglycyl)taxol, 7(N,N-dimethylglycyl)taxol, 2′,7-di-(N,N-dimethylglycyl)taxol, 7(N,N-diethylaminopropionyl)taxol, 2′,7-di(N,N-diethylaminopropionyl)taxol, 2′-(L-glycyl)taxol, 7-(L-glycyl)taxol, 2′,7-di(L-glycyl)taxol, 2′-(L-alanyl)taxol, 7-(L-alanyl)taxol, 2′,7-di(L-alanyl)taxol, 2′-(L-leucyl)taxol, 7-(L-leucyl)taxol, 2′,7-di(L-leucyl)taxol, 2′-(L-isoleucyl)taxol, 7-(L-isoleucyl)taxol, 2′,7-di(L-isoleucyl)taxol, 2′-(L-valyl)taxol, 7-(L-valyl)taxol, 2′7-di(L-valyl)taxol, 2′-(L-phenylalanyl)taxol, 7-(L-phenylalanyl)taxol, 2′,7-di(L-phenylalanyl)taxol, 2′-(L-prolyl)taxol, 7-(L-prolyl)taxol, 2′, 7-di(L-prolyl)taxol, 2′-(L-lysyl)taxol, 7-(L-lysyl)taxol, 2′,7-di(L-lysyl)taxol, 2′-(L-glutamyl)taxol, 7-(L-glutamyl)taxol, 2′,7-di(L-glutamyl)taxol, 2′-(L-arginyl)taxol, 7-(L-arginyl)taxol, 2′,7-di(L-arginyl)taxol}, taxol analogues with modified phenylisoserine side chains, TAXOTERE, (N-debenzoyl-N-tert-(butoxycaronyl)-10-deacetyltaxol, and taxanes (e.g., baccatin III, cephalomannine, 10-deacetylbaccatin III, brevifoliol, yunantaxusin and taxusin); and other taxane analogues and derivatives, including 14-beta-hydroxy-10 deacetybaccatin III, debenzoyl-2-acyl paclitaxel derivatives, benzoate paclitaxel derivatives, phosphonooxy and carbonate paclitaxel derivatives, sulfonated 2′-acryloyltaxol; sulfonated 2′-O-acyl acid paclitaxel derivatives, 18-site-substituted paclitaxel derivatives, chlorinated paclitaxel analogues, C4 methoxy ether paclitaxel derivatives, sulfonamide taxane derivatives, brominated paclitaxel analogues, Girard taxane derivatives, nitrophenyl paclitaxel, 10-deacetylated substituted paclitaxel derivatives, 14-beta-hydroxy-10 deacetylbaccatin III taxane derivatives, C7 taxane derivatives, C10 taxane derivatives, 2-debenzoyl-2-acyl taxane derivatives, 2-debenzoyl and -2-acyl paclitaxel derivatives, taxane and baccatin III analogues bearing new C2 and C4 functional groups, n-acyl paclitaxel analogues, 10-deacetylbaccatin III and 7-protected-10-deacetylbaccatin III derivatives from 10-deacetyl taxol A, 10-deacetyl taxol B, and 10-deacetyl taxol, benzoate derivatives of taxol, 2-aroyl-4-acyl paclitaxel analogues, orthro-ester paclitaxel analogues, 2-aroyl-4-acyl paclitaxel analogues and 1-deoxy paclitaxel and 1-deoxy paclitaxel analogues.

In one aspect, the cell cycle inhibitor is a taxane having the formula (C1): 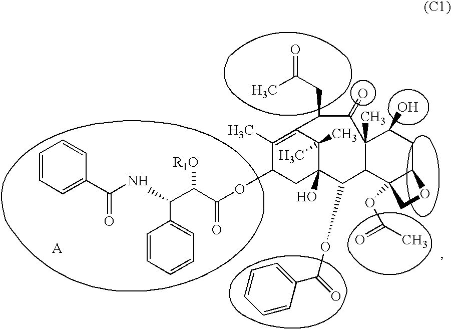
where the gray-highlighted portions may be substituted and the non-highlighted portion is the taxane core. A side-chain (labeled “A” in the diagram) is desirably present in order for the compound to have good activity as a cell cycle inhibitor. Examples of compounds having this structure include paclitaxel (Merck Index entry 7117), docetaxol (TAXOTERE, Merck Index entry 3458), and 3′-desphenyl-3′-(4-ntirophenyl)-N-debenzoyl-N-(t-butoxycarbonyl)-10-deacetyltaxol.

In one aspect, suitable taxanes such as paclitaxel and its analogues and derivatives are disclosed in U.S. Pat. No. 5,440,056 as having the structure (C2): 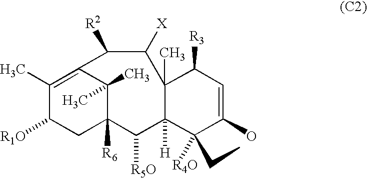
wherein X may be oxygen (paclitaxel), hydrogen (9-deoxy derivatives), thioacyl, or dihydroxyl precursors; R₁ is selected from paclitaxel or TAXOTERE side chains or alkanoyl of the formula (C3) 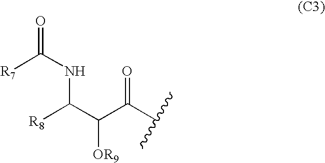
wherein R₇ is selected from hydrogen, alkyl, phenyl, alkoxy, amino, phenoxy (substituted or unsubstituted); R₈ is selected from hydrogen, alkyl, hydroxyalkyl, alkoxyalkyl, aminoalkyl, phenyl (substituted or unsubstituted), alpha or beta-naphthyl; and R₉ is selected from hydrogen, alkanoyl, substituted alkanoyl, and aminoalkanoyl; where substitutions refer to hydroxyl, sulfhydryl, allalkoxyl, carboxyl, halogen, thioalkoxyl, N,N-dimethylamino, alkylamino, dialkylamino, nitro, and —OSO₃H, and/or may refer to groups containing such substitutions; R₂ is selected from hydrogen or oxygen-containing groups, such as hydrogen, hydroxyl, alkoyl, alkanoyloxy, aminoalkanoyloxy, and peptidyalkanoyloxy; R₃ is selected from hydrogen or oxygen-containing groups, such as hydrogen, hydroxyl, alkoyl, alkanoyloxy, aminoalkanoyloxy, and peptidyalkanoyloxy, and may further be a silyl containing group or a sulphur containing group; R₄ is selected from acyl, alkyl, alkanoyl, aminoalkanoyl, peptidylalkanoyl and aroyl; R₅ is selected from acyl, alkyl, alkanoyl, aminoalkanoyl, peptidylalkanoyl and aroyl; R₆ is selected from hydrogen or oxygen-containing groups, such as hydrogen, hydroxyl alkoyl, alkanoyloxy, aminoalkanoyloxy, and peptidyalkanoyloxy.

In one aspect, the paclitaxel analogues and derivatives useful as cell cycle inhibitors are disclosed in PCT International Patent Application No. WO 93/10076. As disclosed in this publication, the analogue or derivative should have a side chain attached to the taxane nucleus at C₁₃, as shown in the structure below (formula C4), in order to confer antitumor activity to the taxane.

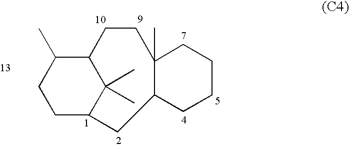

WO 93/10076 discloses that the taxane nucleus may be substituted at any position with the exception of the existing methyl groups. The substitutions may include, for example, hydrogen, alkanoyloxy, alkenoyloxy, aryloyloxy. In addition, oxo groups may be attached to carbons labeled 2, 4, 9, and/or 10. As well, an oxetane ring may be attached at carbons 4 and 5. As well, an oxirane ring may be attached to the carbon labeled 4.

In one aspect, the taxane-based cell cycle inhibitor useful in the present invention is disclosed in U.S. Pat. No. 5,440,056, which discloses 9-deoxo taxanes. These are compounds lacking an oxo group at the carbon labeled 9 in the taxane structure shown above (formula C4). The taxane ring may be substituted at the carbons labeled 1, 7 and 10 (independently) with H, OH, O—R, or O—CO—R where R is an alkyl or an aminoalkyl. As well, it may be substituted at carbons labeled 2 and 4 (independently) with aryol, alkanoyl, aminoalkanoyl or alkyl groups. The side chain of formula (C3) may be substituted at R₇ and R₈ (independently) with phenyl rings, substituted phenyl rings, linear alkanes/alkenes, and groups containing H, O or N. R₉ may be substituted with H, or a substituted or unsubstituted alkanoyl group.

Taxanes in general, and paclitaxel is particular, is considered to function as a cell cycle inhibitor by acting as an anti-microtubule agent, and more specifically as a stabilizer. These compounds have been shown useful in the treatment of proliferative disorders, including: non-small cell (NSC) lung; small cell lung; breast; prostate; cervical; endometrial; head and neck cancers.

In another aspect, the anti-microtuble agent (microtubule inhibitor) is albendazole (carbamic acid, [5-(propylthio)-1H-benzimidazol-2-yl]-, methyl ester), LY-355703 (1,4-dioxa-8,11-diazacyclohexadec-13-ene-2,5,9,12-tetrone, 10-[(3-chloro-4-methoxyphenyl)methyl]-6,6-dimethyl-3-(2-methylpropyl)-16-[(1S)-1-[(2S,3R)-3-phenyloxiranyl]ethyl]-, (3S,10R,13E,16S)-), vindesine (vincaleukoblastine, 3-(aminocarbonyl)-O4-deacetyl-3-de(methoxycarbonyl)-), or WAY-174286.

In another aspect, the cell cycle inhibitor is a vinca alkaloid. Vinca alkaloids have the following general structure. They are indole-dihydroindole dimers.

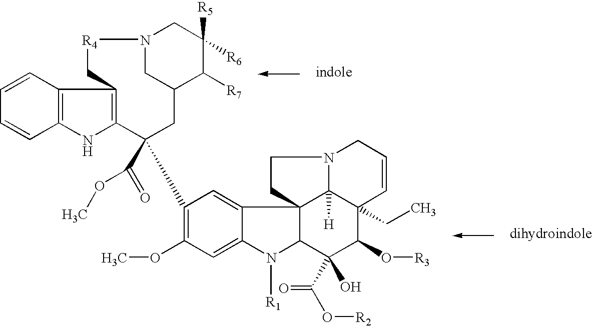

As disclosed in U.S. Pat. Nos. 4,841,045 and 5,030,620, R₁ can be a formyl or methyl group or alternately H. R₁ can also be an alkyl group or an aldehyde-substituted alkyl (e.g., CH₂CHO). R₂ is typically a CH₃ or NH₂ group. However it can be alternately substituted with a lower alkyl ester or the ester linking to the dihydroindole core may be substituted with C(O)—R where R is NH₂, an amino acid ester or a peptide ester. R₃ is typically C(O)CH₃, CH₃ or H. Alternately, a protein fragment may be linked by a bifunctional group, such as maleoyl amino acid. R₃ can also be substituted to form an alkyl ester which may be further substituted. R₄ may be —CH₂— or a single bond. R₅ and R₆ may be H, OH or a lower alkyl, typically —CH₂CH₃. Alternatively R₆ and R₇ may together form an oxetane ring. R₇ may alternately be H. Further substitutions include molecules wherein methyl groups are substituted with other alkyl groups, and whereby unsaturated rings may be derivatized by the addition of a side group such as an alkane, alkene, alkyne, halogen, ester, amide or amino group.

Exemplary vinca alkaloids are vinblastine, vincristine, vincristine sulfate, vindesine, and vinorelbine, having the structures:

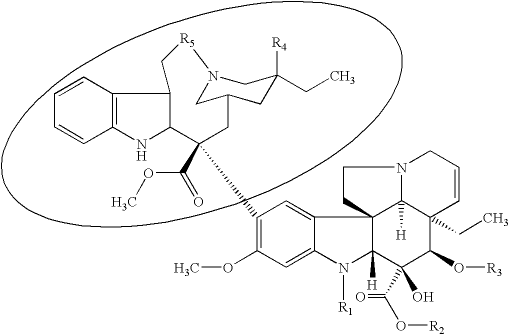

| | R₁ | R₂ | R₃ | R₄ | R₅ |
|---|---|---|---|---|---|
| Vinblastine: | CH₃ | CH₃ | C(O)CH₃ | OH | CH₂ |
| Vincristine: | CH₂O | CH₃ | C(O)CH₃ | OH | CH₂ |
| Vindesine: | CH₃ | NH₂ | H | OH | CH₂ |
| Vinorelbine: | CH₃ | CH₃ | CH₃ | H | single bond |

Analogues typically require the side group (shaded area) in order to have activity. These compounds are thought to act as cell cycle inhibitors by functioning as anti-microtubule agents, and more specifically to inhibit polymerization. These compounds have been shown useful in treating proliferative disorders, including NSC lung; small cell lung; breast; prostate; brain; head and neck; retinoblastoma; bladder; and penile cancers; and soft tissue sarcoma.

In another aspect, the cell cycle inhibitor is a camptothecin, or an analog or derivative thereof. Camptothecins have the following general structure.

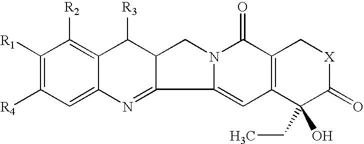

In this structure, X is typically O, but can be other groups, e.g., NH in the case of 21-lactam derivatives. R₁ is typically H or OH, but may be other groups, e.g., a terminally hydroxylated C₁₋₃ alkane. R₂ is typically H or an amino containing group such as (CH₃)₂NHCH₂, but may be other groups e.g., NO₂, NH₂, halogen (as disclosed in, e.g., U.S. Pat. No. 5,552,156) or a short alkane containing these groups. R₃ is typically H or a short alkyl such as C₂H₅. R₄ is typically H but may be other groups, e.g., a methylenedioxy group with R₁.

Exemplary camptothecin compounds include topotecan, irinotecan (CPT-11), 9-aminocamptothecin, 21-lactam-20(S)-camptothecin, 10,11-methylenedioxycamptothecin, SN-38, 9-nitrocamptothecin, 10-hydroxycamptothecin. Exemplary compounds have the structures:

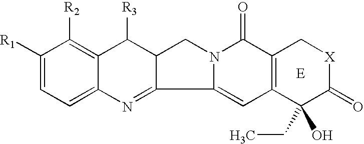

| | R₁ | R₂ | R₃ |
|---|---|---|---|
| Camptothecin: | H | H | H |
| Topotecan: | OH | (CH₃)₂NHCH₂ | H |
| SN-38: | OH | H | C₂H₅ |

X: O for most analogs, NH for 21-lactam analogs

Camptothecins have the five rings shown here. The ring labeled E must be intact (the lactone rather than carboxylate form) for maximum activity and minimum toxicity. These compounds are useful to as cell cycle inhibitors, where they can function as topoisomerase I inhibitors and/or DNA cleavage agents. They have been shown useful in the treatment of proliferative disorders, including, for example, NSC lung; small cell lung; and cervical cancers.

In another aspect, the cell cycle inhibitor is a podophyllotoxin, or a derivative or an analogue thereof. Exemplary compounds of this type are etoposide or teniposide, which have the following structures:

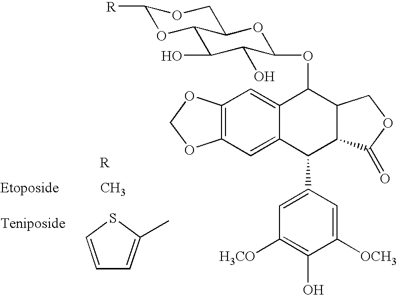

These compounds are thought to function as cell cycle inhibitors by being topoisomerase II inhibitors and/or by DNA cleaving agents. They have been shown useful as antiproliferative agents in, e.g., small cell lung, prostate, and brain cancers, and in retinoblastoma.

Another example of a DNA topoisomerase inhibitor is lurtotecan dihydrochloride (11H-1,4-dioxino[2,3-g]pyrano[3′,4′:6,7]indolizino[1,2-b]quinoline-9,12(8H,14H)-dione, 8-ethyl-2,3-dihydro-8-hydroxy-15-[(4-methyl-1-piperazinyl)methyl]-, dihydrochloride, (S)-).

In another aspect, the cell cycle inhibitor is an anthracycline. Anthracyclines have the following general structure, where the R groups may be a variety of organic groups:

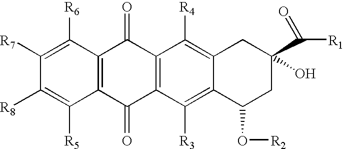

According to U.S. Pat. No. 5,594,158, suitable R groups are: R₁ is CH₃ or CH₂OH; R₂ is daunosamine or H; R₃ and R₄ are independently one of OH, NO₂, NH₂, F, Cl, Br, I, CN, H or groups derived from these; R₅₋₇ are all H or R₅ and R₆ are H and R₇ and R₈ are alkyl or halogen, or vice versa: R₇ and R₈ are H and R₅ and R₆ are alkyl or halogen.

According to U.S. Pat. No. 5,843,903, R₂ may be a conjugated peptide. According to U.S. Pat. Nos. 4,215,062 and 4,296,105, R₅ may be OH or an ether linked alkyl group. R₁ may also be linked to the anthracycline ring by a group other than C(O), such as an alkyl or branched alkyl group having the C(O) linking moiety at its end, such as —CH₂CH(CH₂—X)C(O)—R₁, wherein X is H or an alkyl group (see, e.g., U.S. Pat. No. 4,215,062). R₂ may alternately be a group linked by the functional group ═N—NHC(O)—Y, where Y is a group such as a phenyl or substituted phenyl ring. Alternately R₃ may have the following structure: 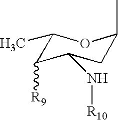
in which R₉ is OH either in or out of the plane of the ring, or is a second sugar moiety such as R₃. R₁₀ may be H or form a secondary amine with a group such as an aromatic group, saturated or partially saturated 5 or 6 membered heterocyclic having at least one ring nitrogen (see U.S. Pat. No. 5,843,903). Alternately, R₁₀ may be derived from an amino acid, having the structure —C(O)CH(NHR₁₁)(R₁₂), in which R₁₁ is H, or forms a C₃₋₄ membered alkylene with R₁₂. R₁₂ may be H, alkyl, aminoalkyl, amino, hydroxy, mercapto, phenyl, benzyl or methylthio (see U.S. Pat. No. 4,296,105).

Exemplary anthracyclines are doxorubicin, daunorubicin, idarubicin, epirubicin, pirarubicin, zorubicin, and carubicin. Suitable compounds have the structures:

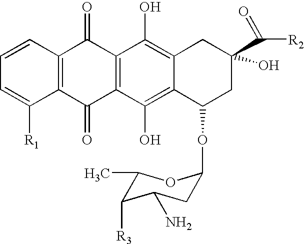

| | R₁ | R₂ | R₃ |
|---|---|---|---|
| Doxorubicin: | OCH₃ | CH₂OH | OH out of ring plane |
| Epirubicin: | OCH₃ | CH₂OH | OH in ring plane |
| (4′ epimer | | | |
| of doxorubicin) | | | |
| Daunorubicin: | OCH₃ | CH₃ | OH out of ring plane |
| Idarubicin: | H | CH₃ | OH out of ring plane |
| Pirarubicin | OCH₃ | OH | A |
| Zorubicin | OCH₃ | ═N—NHC(O)C₈H₅ | B |
| Cerubicin | OH | CH₃ | B |

A:

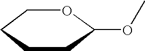

B:

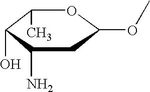

Other suitable anthracyclines are anthramycin, mitoxantrone, menogaril, nogalamycin, aclacinomycin A, olivomycin A, chromomycin A₃, and plicamycin having the structures:

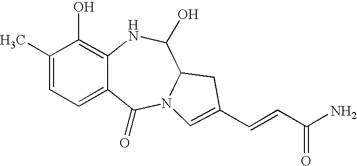

Anthramycin

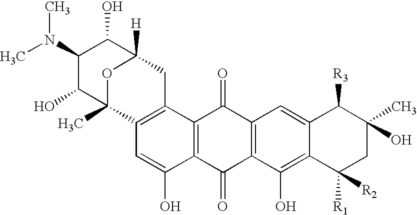

| | R₁ | R₂ | R₃ |
|---|---|---|---|
| Menogaril | H | OCH₃ | H |
| Nogalamycin | O-sugar | H | COOCH₃ |
| sugar: | 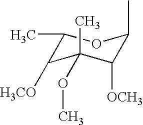 | | |

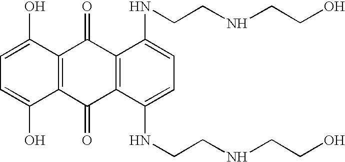

Mitoxantrone

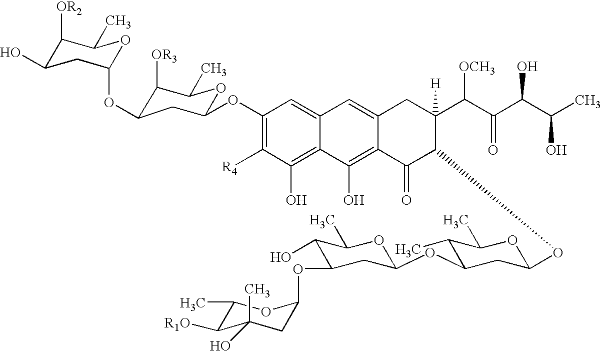

| | | | | |
|---|---|---|---|---|
| | R₁ | R₂ | R₃ | R₄ |
| Olivomycin A | COCH(CH₃)₂ | CH₃ | COCH₃ | H |
| Chromomycin A₃ | COCH₃ | CH₃ | COCH₃ | CH₃ |
| Plicamycin | H | H | H | CH₃ |

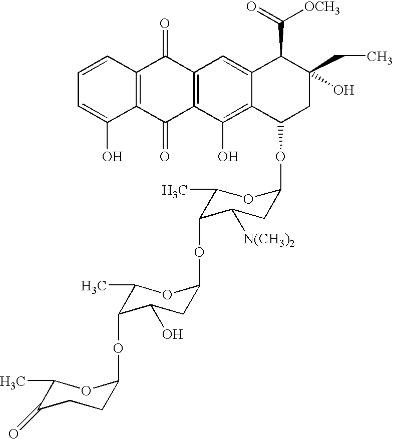

Aclacinomycin A

These compounds are thought to function as cell cycle inhibitors by being topoisomerase inhibitors and/or by DNA cleaving agents. They have been shown useful in the treatment of proliferative disorders, including small cell lung; breast; endometrial; head and neck; retinoblastoma; liver; bile duct; islet cell; and bladder cancers; and soft tissue sarcoma.

In another aspect, the cell cycle inhibitor is a platinum compound. In general, suitable platinum complexes may be of Pt(II) or Pt(IV) and have this basic structure: 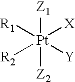
wherein X and Y are anionic leaving groups such as sulfate, phosphate, carboxylate, and halogen; R₁ and R₂ are alkyl, amine, amino alkyl any may be further substituted, and are basically inert or bridging groups. For Pt(II) complexes Z₁ and Z₂ are non-existent. For Pt(IV) Z₁ and Z₂ may be anionic groups such as halogen, hydroxy, carboxylate, ester, sulfate or phosphate. See, e.g., U.S. Pat. Nos. 4,588,831 and 4,250,189.

Suitable platinum complexes may contain multiple Pt atoms. See, e.g., U.S. Pat. Nos. 5,409,915 and 5,380,897. For example bisplatinum and triplatinum complexes of the type:

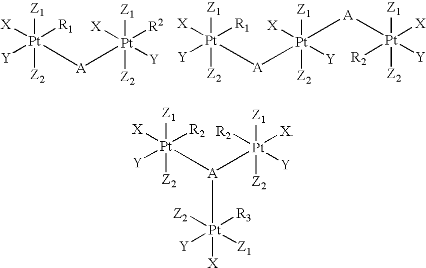

Exemplary platinum compounds are cisplatin, carboplatin, oxaliplatin, and miboplatin having the structures:

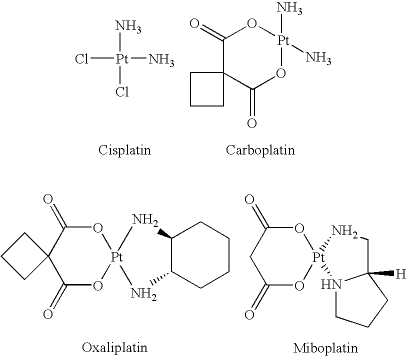

These compounds are thought to function as cell cycle inhibitors by binding to DNA, i.e., acting as alkylating agents of DNA. These compounds have been shown useful in the treatment of cell proliferative disorders, including, e.g., NSC lung; small cell lung; breast; cervical; brain; head and neck; esophageal; retinoblastom; liver; bile duct; bladder; penile; and vulvar cancers; and soft tissue sarcoma.

In another aspect, the cell cycle inhibitor is a nitrosourea. Nitrosourease have the following general structure (C5), where typical R groups are shown below.

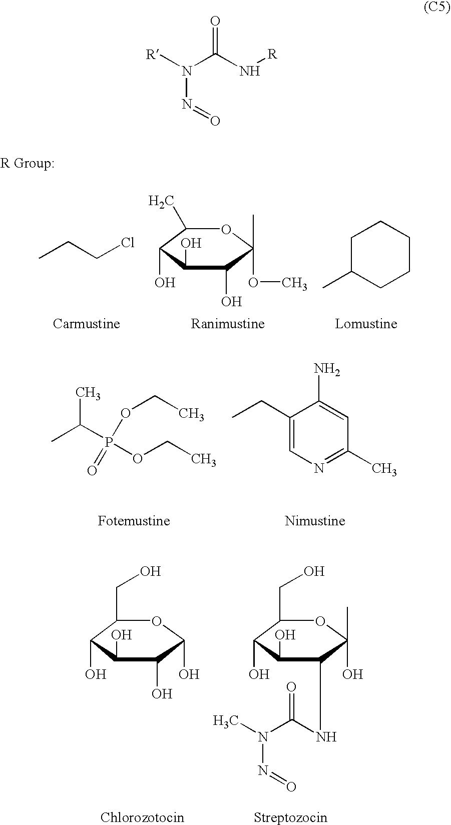

Other suitable R groups include cyclic alkanes, alkanes, halogen substituted groups, sugars, aryl and heteroaryl groups, phosphonyl and sulfonyl groups. As disclosed in U.S. Pat. No. 4,367,239, R may suitably be CH₂—C(X)(Y)(Z), wherein X and Y may be the same or different members of the following groups: phenyl, cyclyhexyl, or a phenyl or cyclohexyl group substituted with groups such as halogen, lower alkyl (C₁₋₄), trifluore methyl, cyano, phenyl, cyclohexyl, lower alkyloxy (C₁₋₄). Z has the following structure: -alkylene-N—R₁R₂, where R₁ and R₂ may be the same or different members of the following group: lower alkyl (C₁₋₄) and benzyl, or together R₁ and R₂ may form a saturated 5 or 6 membered heterocyclic such as pyrrolidine, piperidine, morfoline, thiomorfoline, N-lower alkyl piperazine, where the heterocyclic may be optionally substituted with lower alkyl groups.

As disclosed in U.S. Pat. No. 6,096,923, R and R′ of formula (C5) may be the same or different, where each may be a substituted or unsubstituted hydrocarbon having 1-10 carbons. Substitutions may include hydrocarbyl, halo, ester, amide, carboxylic acid, ether, thioether and alcohol groups. As disclosed in U.S. Pat. No. 4,472,379, R of formula (C5) may be an amide bond and a pyranose structure (e.g., methyl 2′-(N-(N-(2-chloroethyl)-N-nitroso-carbamoyl)-glycyl)amino-2′-deoxy-α-D-glucopyranoside). As disclosed in U.S. Pat. No. 4,150,146, R of formula (C5) may be an alkyl group of 2 to 6 carbons and may be substituted with an ester, sulfonyl, or hydroxyl group. It may also be substituted with a carboxylic acid or CONH₂ group.

Exemplary nitrosoureas are BCNU (carmustine), methyl-CCNU (semustine), CCNU (lomustine), ranimustine, nimustine, chlorozotocin, fotemustine, and streptozocin, having the structures:

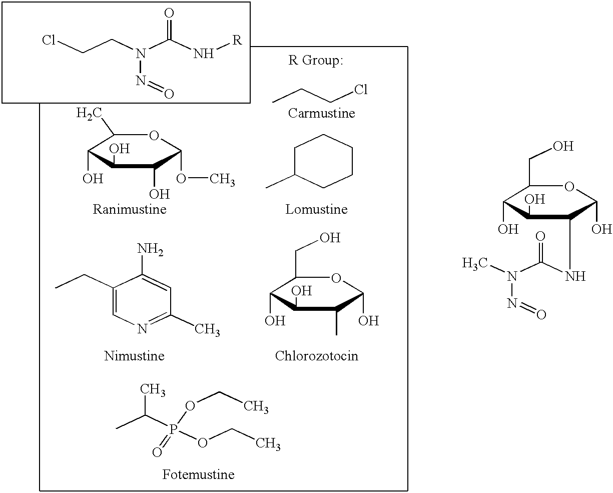

These nitrosourea compounds are thought to function as cell cycle inhibitors by binding to DNA, that is, by functioning as DNA alkylating agents. These cell cycle inhibitors have been shown useful in treating cell proliferative disorders such as, for example, islet cell; small cell lung; melanoma; and brain cancers.

In another aspect, the cell cycle inhibitor is a nitroimidazole, where exemplary nitroimidazoles are metronidazole, benznidazole, etanidazole, and misonidazole, having the structures:

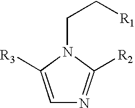

| | R₁ | R₂ | R₃ |
|---|---|---|---|
| Metronidazole | OH | CH₃ | NO₂ |
| Benznidazole | C(O)NHCH₂-benzyl | NO₂ | H |
| Etanidazole | CONHCH₂CH₂OH | NO₂ | H |

Suitable nitroimidazole compounds are disclosed in, e.g., U.S. Pat. Nos. 4,371,540 and 4,462,992.

In another aspect, the cell cycle inhibitor is a folic acid antagonist, such as methotrexate or derivatives or analogues thereof, including edatrexate, trimetrexate, raltitrexed, piritrexim, denopterin, tomudex, and pteropterin. Methotrexate analogues have the following general structure:

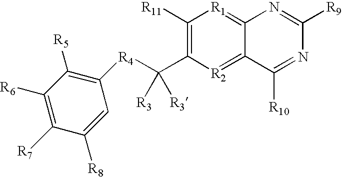

The identity of the R group may be selected from organic groups, particularly those groups set forth in U.S. Pat. Nos. 5,166,149 and 5,382,582. For example, R₁ may be N, R₂ may be N or C(CH₃), R₃ and R₃′ may H or alkyl, e.g., CH₃, R₄ may be a single bond or NR, where R is H or alkyl group. R_{5,6,8} may be H, OCH₃, or alternately they can be halogens or hydro groups. R₇ is a side chain of the general structure: 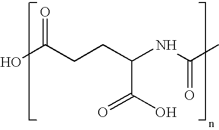
wherein n=1 for methotrexate, n=3 for pteropterin. The carboxyl groups in the side chain may be esterified or form a salt such as a Zn²⁺ salt. R₉ and R₁₀ can be NH₂ or may be alkyl substituted.

Exemplary folic acid antagonist compounds have the structures:

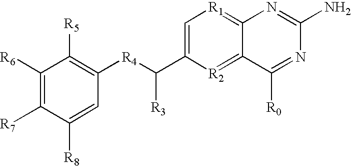

| | R₀ | R₁ | R₂ | R₃ | R₄ | R₅ | R₆ | R₇ | R₈ |
|---|---|---|---|---|---|---|---|---|---|
| Methotrexate | NH₂ | N | N | H | N(CH₃) | H | H | A(n = 1) | H |
| Edatrexate | NH₂ | N | N | H | N(CH₃CH₃) | H | H | A(n = 1) | H |
| Trimatraxate | NH₂ | N | C(CH₃) | H | NH | H | OCH₃ | OCH₃ | OCH₃ |
| Pteropterin | NH₂ | N | N | H | N(CH₃) | H | H | A(n = 3) | H |
| Denopterin | OH | N | N | CH₃ | N(CH₃) | H | H | A(n = 1) | H |
| Piritrexim | NH₂ | N | C(CH₃)H | Single | OCH₃ | H | H | OCH₃ | H |
| | | | | bond | | | | | |

A:

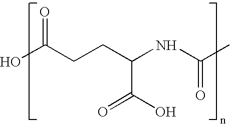

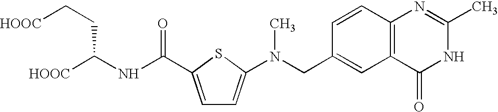

Tomudex

These compounds are thought to function as cell cycle inhibitors by serving as antimetabolites of folic acid. They have been shown useful in the treatment of cell proliferative disorders including, for example, soft tissue sarcoma, small cell lung, breast, brain, head and neck, bladder, and penile cancers.

In another aspect, the cell cycle inhibitor is a cytidine analogue, such as cytarabine or derivatives or analogues thereof, including enocitabine, FMdC ((E(−2′-deoxy-2′-(fluoromethylene)cytidine), gemcitabine, 5-azacitidine, ancitabine, and 6-azauridine. Exemplary compounds have the structures:

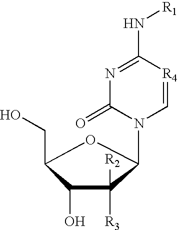

| | R₁ | R₂ | R₃ | R₄ |
|---|---|---|---|---|
| Cytarabine | H | OH | H | CH |
| Enocitabine | C(O)(CH₂)₂₀CH₃ | OH | H | CH |
| Gemcitabine | H | F | F | CH |
| Azacitidine | H | H | OH | N |
| FMdC | H | CH₂F | H | CH |

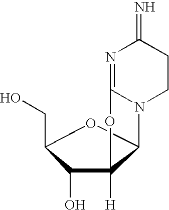

Ancitabine

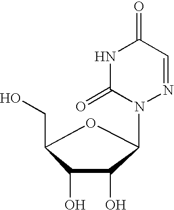

6-Azauridine

These compounds are thought to function as cell cycle inhibitors as acting as antimetabolites of pyrimidine. These compounds have been shown useful in the treatment of cell proliferative disorders including, for example, pancreatic, breast, cervical, NSC lung, and bile duct cancers.

In another aspect, the cell cycle inhibitor is a pyrimidine analogue. In one aspect, the pyrimidine analogues have the general structure: 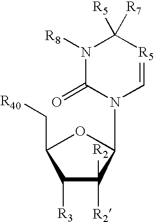
wherein positions 2′, 3′ and 5′ on the sugar ring (R₂, R₃ and R₄, respectively) can be H, hydroxyl, phosphoryl (see, e.g., U.S. Pat. No. 4,086,417) or ester (see, e.g., U.S. Pat. No. 3,894,000). Esters can be of alkyl, cycloalkyl, aryl or heterocyclo/aryl types. The 2′ carbon can be hydroxylated at either R₂ or R₂′, the other group is H. Alternately, the 2′ carbon can be substituted with halogens e.g., fluoro or difluoro cytidines such as Gemcytabine. Alternately, the sugar can be substituted for another heterocyclic group such as a furyl group or for an alkane, an alkyl ether or an amide linked alkane such as C(O)NH(CH₂)₅CH₃. The 2° amine can be substituted with an aliphatic acyl (R₁) linked with an amide (see, e.g., U.S. Pat. No. 3,991,045) or urethane (see, e.g., U.S. Pat. No. 3,894,000) bond. It can also be further substituted to form a quaternary ammonium salt. R₅ in the pyrimidine ring may be N or CR, where R is H, halogen containing groups, or alkyl (see, e.g., U.S. Pat. No. 4,086,417). R₆ and R₇ can together can form an oxo group or R₆=—NH—R₁ and R₇═H. R₈ is H or R₇ and R₈ together can form a double bond or R₈ can be X, where X is:

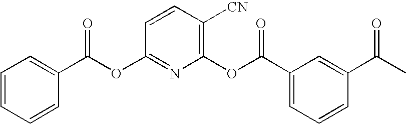

Specific pyrimidine analogues are disclosed in U.S. Pat. No. 3,894,000 (see, e.g., 2′-O-palmityl-ara-cytidine, 3′-O-benzoyl-ara-cytidine, and more than 10 other examples); U.S. Pat. No. 3,991,045 (see, e.g., N4-acyl-1-β-D-arabinofuranosylcytosine, and numerous acyl groups derivatives as listed therein, such as palmitoyl.

In another aspect, the cell cycle inhibitor is a fluoropyrimidine analogue, such as 5-fluorouracil, or an analogue or derivative thereof, including carmofur, doxifluridine, emitefur, tegafur, and floxuridine. Exemplary compounds have the structures:

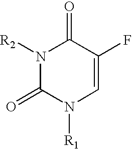

| | R₁ | R₂ |
|---|---|---|
| 5-Fluorouracil | H | H |
| Carmofur | C(O)NH(CH₂)₅CH₃ | H |
| Doxifluridine | A₁ | H |
| Floxuridine | A₂ | H |
| Emitefur | CH₂OH₂CH₃ | B |
| Tegafur | | H |

A₁

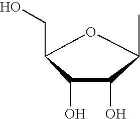

A₂

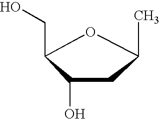

B

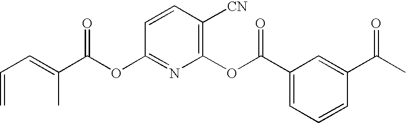

C

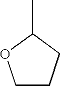

Other suitable fluoropyrimidine analogues include 5-FudR (5-fluoro-deoxyuridine), or an analogue or derivative thereof, including 5-iododeoxyuridine (5-IudR), 5-bromodeoxyuridine (5-BudR), fluorouridine triphosphate (5-FUTP), and fluorodeoxyuridine monophosphate (5-dFUMP). Exemplary compounds have the structures:

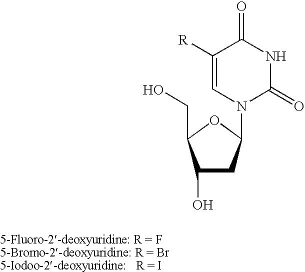

These compounds are thought to function as cell cycle inhibitors by serving as antimetabolites of pyrimidine. These compounds have been shown useful in the treatment of cell proliferative disorders such as breast, cervical, non-melanoma skin, head and neck, esophageal, bile duct, pancreatic, islet cell, penile, and vulvar cancers.

In another aspect, the cell cycle inhibitor is a purine analogue. Purine analogues have the following general structure. 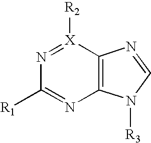
wherein X is typically carbon; R₁ is H, halogen, amine or a substituted phenyl; R₂ is H, a primary, secondary or tertiary amine, a sulfur containing group, typically —SH, an alkane, a cyclic alkane, a heterocyclic or a sugar; R₃ is H, a sugar (typically a furanose or pyranose structure), a substituted sugar or a cyclic or heterocyclic alkane or aryl group. See, e.g., U.S. Pat. No. 5,602,140 for compounds of this type.

In the case of pentostatin, X—R2 is —CH₂CH(OH)—. In this case a second carbon atom is inserted in the ring between X and the adjacent nitrogen atom. The X—N double bond becomes a single bond.

U.S. Pat. No. 5,446,139 describes suitable purine analogues of the type shown in the formula. 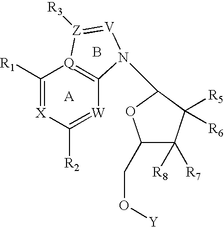
wherein N signifies nitrogen and V, W, X, Z can be either carbon or nitrogen with the following provisos. Ring A may have 0 to 3 nitrogen atoms in its structure. If two nitrogens are present in ring A, one must be in the W position. If only one is present, it must not be in the Q position. V and Q must not be simultaneously nitrogen. Z and Q must not be simultaneously nitrogen. If Z is nitrogen, R₃ is not present. Furthermore, R₁₋₃ are independently one of H, halogen, C₁₋₇ alkyl, C₁₋₇ alkenyl, hydroxyl, mercapto, C₁₋₇ alkylthio, C₁₋₇ alkoxy, C₂₋₇ alkenyloxy, aryl oxy, nitro, primary, secondary or tertiary amine containing group. R₅₋₈ are H or up to two of the positions may contain independently one of OH, halogen, cyano, azido, substituted amino, R₅ and R₇ can together form a double bond. Y is H, a C₁₋₇ alkylcarbonyl, or a mono-di or tri phosphate.

Exemplary suitable purine analogues include 6-mercaptopurine, thiguanosine, thiamiprine, cladribine, fludaribine, tubercidin, puromycin, pentoxyfilline; where these compounds may optionally be phosphorylated. Exemplary compounds have the structures:

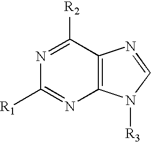

| | R₁ | R₂ | R₃ |
|---|---|---|---|
| 6-Mercaptopurine | H | SH | H |
| Thioguanosine | NH₂ | SH | B₁ |
| Thiamiprine | NH₂ | A | H |
| Cladribine | Cl | NH₂ | B₂ |
| Fludarabine | F | NH₂ | B₃ |
| Puromycin | H | N(CH₃)₂ | B₄ |
| Tubercidin | H | NH₂ | B₁ |

A:

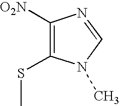

B₁:

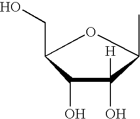

B₂:

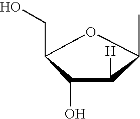

B₃:

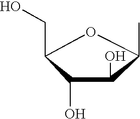

B₄:

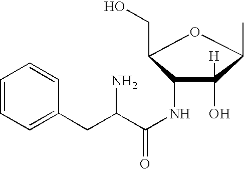

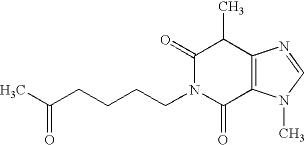

Pentoxyfilline

These compounds are thought to function as cell cycle inhibitors by serving as antimetabolites of purine.

In another aspect, the cell cycle inhibitor is a nitrogen mustard. Many suitable nitrogen mustards are known and are suitably used as a cell cycle inhibitor in the present invention. Suitable nitrogen mustards are also known as cyclophosphamides.

A preferred nitrogen mustard has the general structure: 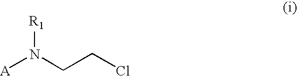
Where A is: 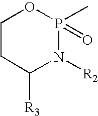
or —CH₃ or other alkane, or chloronated alkane, typically CH₂CH(CH₃)Cl, or a polycyclic group such as B, or a substituted phenyl such as C or a heterocyclic group such as D.

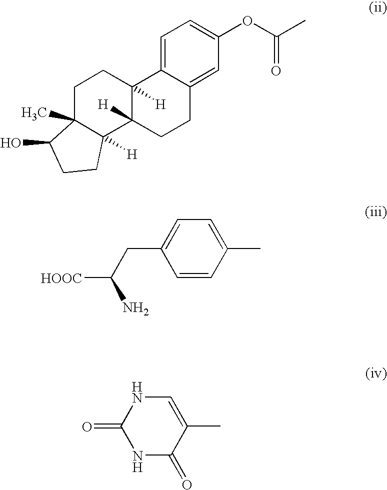

Examples of suitable nitrogen mustards are disclosed in U.S. Pat. No. 3,808,297, wherein A is: 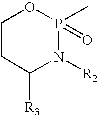
R₁₋₂ are H or CH₂CH₂Cl; R₃ is H or oxygen-containing groups such as hydroperoxy; and R₄ can be alkyl, aryl, heterocyclic.

The cyclic moiety need not be intact. See, e.g., U.S. Pat. Nos. 5,472,956, 4,908,356, 4,841,085 that describe the following type of structure: 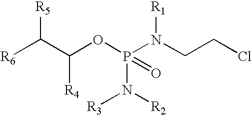
wherein R₁ is H or CH₂CH₂Cl, and R₂₆ are various substituent groups.

Exemplary nitrogen mustards include methylchloroethamine, and analogues or derivatives thereof, including methylchloroethamine oxide hydrohchloride, novembichin, and mannomustine (a halogenated sugar). Exemplary compounds have the structures:

| 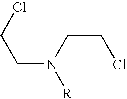 | | 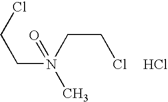 |
|---|---|---|
| | R | |
| Mechlorothanime | CH₃ | Mechlorethanime Oxide HCl |
| Novembichin | CH₂CH(CH₃)Cl | |

The nitrogen mustard may be cyclophosphamide, ifosfamide, perfosfamide, or torofosfamide, where these compounds have the structures:

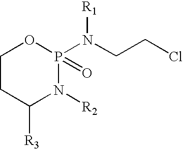

| | R₁ | R₂ | R₃ |
|---|---|---|---|
| Cyclophosphamide | H | CH₂CH₂Cl | H |
| Ifosfamide | CH₂CH₂Cl | H | H |
| Perfosfamide | CH₂CH₂Cl | H | OOH |
| Torofosfamide | CH₂CH₂Cl | CH₂CH₂Cl | H |

The nitrogen mustard may be estramustine, or an analogue or derivative thereof, including phenesterine, prednimustine, and estramustine PO₄. Thus, suitable nitrogen mustard type cell cycle inhibitors of the present invention have the structures:

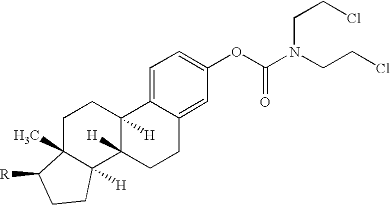

| | R |
|---|---|
| Estramustine | OH |
| Phenesterine | C(CH₃)(CH₂)₃CH(CH₃)₂ |

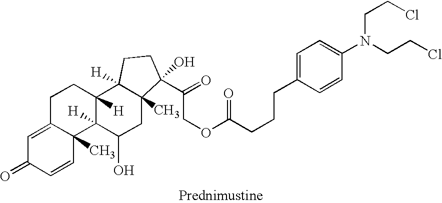

The nitrogen mustard may be chlorambucil, or an analogue or derivative thereof, including melphalan and chlormaphazine. Thus, suitable nitrogen mustard type cell cycle inhibitors of the present invention have the structures:

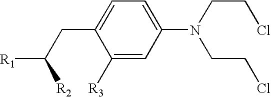

| | R₁ | R₂ | R₃ |
|---|---|---|---|
| Chlorambucil | CH₂COOH | H | H |
| Melphalan | COOH | NH₂ | H |

| | | |
|---|---|---|
| Chlornaphazine | H | together forms a |
| | | benzene ring |

The nitrogen mustard may be uracil mustard, which has the structure:

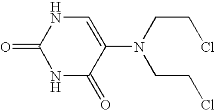

The nitrogen mustards are thought to function as cell cycle inhibitors by serving as alkylating agents for DNA. Nitrogen mustards have been shown useful in the treatment of cell proliferative disorders including, for example, small cell lung, breast, cervical, head and neck, prostate, retinoblastoma, and soft tissue sarcoma.

The cell cycle inhibitor of the present invention may be a hydroxyurea. Hydroxyureas have the following general structure:

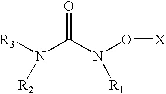

Suitable hydroxyureas are disclosed in, for example, U.S. Pat. No. 6,080,874, wherein R₁ is: 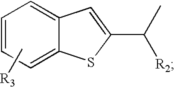
and R₂ is an alkyl group having 1-4 carbons and R₃ is one of H, acyl, methyl, ethyl, and mixtures thereof, such as a methylether.

Other suitable hydroxyureas are disclosed in, e.g., U.S. Pat. No. 5,665,768, wherein R₁ is a cycloalkenyl group, for example N-(3-(5-(4-fluorophenylthio)-furyl)-2-cyclopenten-1-yl)N-hydroxyurea; R₂ is H or an alkyl group having 1 to 4 carbons and R₃ is H; X is H or a cation.

Other suitable hydroxyureas are disclosed in, e.g., U.S. Pat. No. 4,299,778, wherein R₁ is a phenyl group substituted with on or more fluorine atoms; R₂ is a cyclopropyl group; and R₃ and X is H.

Other suitable hydroxyureas are disclosed in, e.g., U.S. Pat. No. 5,066,658, wherein R₂ and R₃ together with the adjacent nitrogen form: 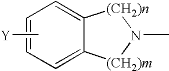
wherein m is 1 or 2, n is 0-2 and Y is an alkyl group.

In one aspect, the hydroxy urea has the structure:

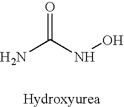

Hydroxyureas are thought to function as cell cycle inhibitors by serving to inhibit DNA synthesis.

In another aspect, the cell cycle inhibitor is a mytomicin, such as mitomycin C, or an analogue or derivative thereof, such as porphyromycin. Exemplary compounds have the structures:

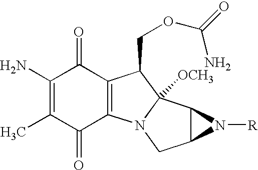

| | R |
|---|---|
| Mitomycin C | H |
| Porphyromycin | CH₃ |
| (N-methyl Mitomycin C) | |

These compounds are thought to function as cell cycle inhibitors by serving as DNA alkylating agents. Mitomycins have been shown useful in the treatment of cell proliferative disorders such as, for example, esophageal, liver, bladder, and breast cancers.

In another aspect, the cell cycle inhibitor is an alkyl sulfonate, such as busulfan, or an analogue or derivative thereof, such as treosulfan, improsulfan, piposulfan, and pipobroman. Exemplary compounds have the structures:

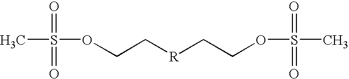

| | R |
|---|---|
| Busulfan | single bond |
| Improsulfan | —CH₂—NH—CH₂— |
| Piposulfan | 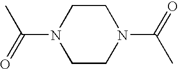 |

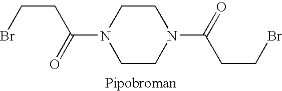

These compounds are thought to function as cell cycle inhibitors by serving as DNA alkylating agents.

In another aspect, the cell cycle inhibitor is a benzamide. In yet another aspect, the cell cycle inhibitor is a nicotinamide. These compounds have the basic structure: 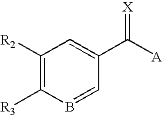
wherein X is either O or S; A is commonly NH₂ or it can be OH or an alkoxy group; B is N or C—R₄, where R₄ is H or an ether-linked hydroxylated alkane such as OCH₂CH₂OH, the alkane may be linear or branched and may contain one or more hydroxyl groups. Alternately, B may be N—R₅ in which case the double bond in the ring involving B is a single bond. R₅ may be H, and alkyl or an aryl group (see, e.g., U.S. Pat. No. 4,258,052); R₂ is H, OR₆, SR₆ or NHR₆, where R₆ is an alkyl group; and R₃ is H, a lower alkyl, an ether linked lower alkyl such as —O-Me or —O-ethyl (see, e.g., U.S. Pat. No. 5,215,738).

Suitable benzamide compounds have the structures: 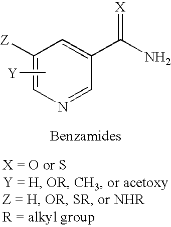
where additional compounds are disclosed in U.S. Pat. No. 5,215,738, (listing some 32 compounds).

Suitable nicotinamide compounds have the structures:

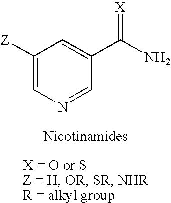

where additional compounds are disclosed in U.S. Pat. No. 5,215,738,

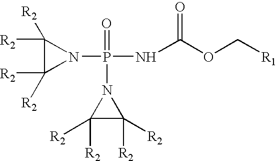

| | R₁ | R₂ |
|---|---|---|
| Benzodepa | phenyl | H |
| Meturedepa | CH₃ | CH₃ |
| Uredepa | CH₃ | H |

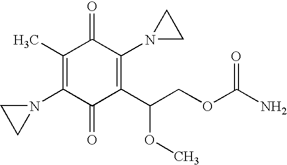

Carboquone

In another aspect, the cell cycle inhibitor is a halogenated sugar, such as mitolactol, or an analogue or derivative thereof, including mitobronitol and mannomustine. Exemplary compounds have the structures:

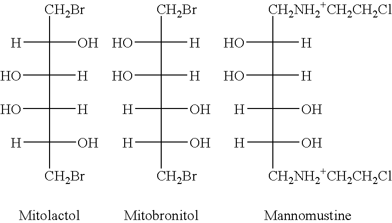

In another aspect, the cell cycle inhibitor is a diazo compound, such as azaserine, or an analogue or derivative thereof, including 6-diazo-5-oxo-L-norleucine and 5-diazouracil (also a pyrimidine analog). Exemplary compounds have the structures:

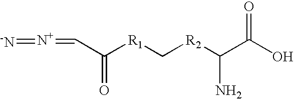

| | R₁ | R₂ |
|---|---|---|
| Azaserine | O | single bond |
| 6-diazo-5-oxo- | single bond | CH₂ |
| L-norleucine | | |

Other compounds that may serve as cell cycle inhibitors according to the present invention are pazelliptine; wortmannin; metoclopramide; RSU; buthionine sulfoxime; tumeric; curcumin; AG337, a thymidylate synthase inhibitor; levamisole; lentinan, a polysaccharide; razoxane, an EDTA analogue; indomethacin; chlorpromazine; α and β interferon; MnBOPP; gadolinium texaphyrin; 4-amino-1,8-naphthalimide; staurosporine derivative of CGP; and SR-2508.

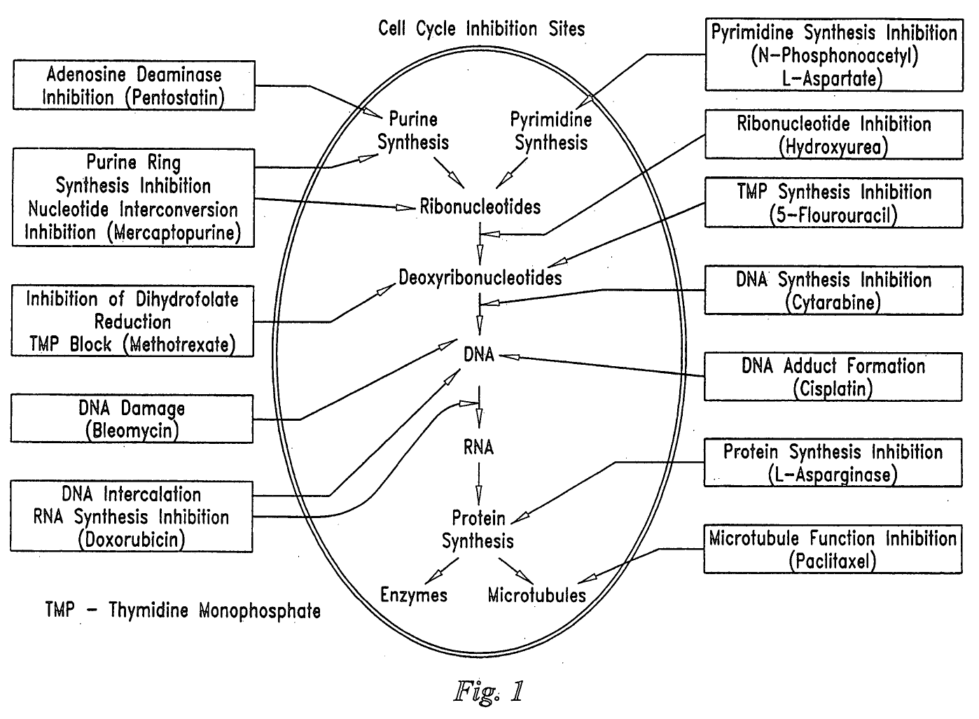

Thus, in one aspect, the cell cycle inhibitor is a DNA alylating agent. In another aspect, the cell cycle inhibitor is an anti-microtubule agent. In another aspect, the cell cycle inhibitor is a topoisomerase inhibitor. In another aspect, the cell cycle inhibitor is a DNA cleaving agent. In another aspect, the cell cycle inhibitor is an antimetabolite. In another aspect, the cell cycle inhibitor functions by inhibiting adenosine deaminase (e.g., as a purine analogue). In another aspect, the cell cycle inhibitor functions by inhibiting purine ring synthesis and/or as a nucleotide interconversion inhibitor (e.g., as a purine analogue such as mercaptopurine). In another aspect, the cell cycle inhibitor functions by inhibiting dihydrofolate reduction and/or as a thymidine monophosphate block (e.g., methotrexate). In another aspect, the cell cycle inhibitor functions by causing DNA damage (e.g., bleomycin). In another aspect, the cell cycle inhibitor functions as a DNA intercalation agent and/or RNA synthesis inhibition (e.g., doxorubicin, aclarubicin, or detorubicin (acetic acid, diethoxy-, 2-[4-[(3-amino-2,3,6-trideoxy-alpha-L-lyxo-hexopyranosyl)oxy]-1,2,3,4,6,11-hexahydro-2,5,12-trihydroxy-7-methoxy-6,11-dioxo-2-naphthacenyl]-2-oxoethyl ester, (2S-cis)-)). In another aspect, the cell cycle inhibitor functions by inhibiting pyrimidine synthesis (e.g., N-phosphonoacetyl-L-aspartate). In another aspect, the cell cycle inhibitor functions by inhibiting ribonucleotides (e.g., hydroxyurea). In another aspect, the cell cycle inhibitor functions by inhibiting thymidine monophosphate (e.g., 5-fluorouracil). In another aspect, the cell cycle inhibitor functions by inhibiting DNA synthesis (e.g., cytarabine). In another aspect, the cell cycle inhibitor functions by causing DNA adduct formation (e.g., platinum compounds). In another aspect, the cell cycle inhibitor functions by inhibiting protein synthesis (e.g., L-asparginase). In another aspect, the cell cycle inhibitor functions by inhibiting microtubule function (e.g., taxanes). In another aspect, the cell cycle inhibitor acts at one or more of the steps in the biological pathway shown in FIG. 1.

Additional cell cycle inhibitor s useful in the present invention, as well as a discussion of the mechanisms of action, may be found in Hardman J. G., Limbird L. E. Molinoff R. B., Ruddon R W., Gilman A. G. editors, Chemotherapy of Neoplastic Diseases in Goodman and Gilman's The Pharmacological Basis of Therapeutics Ninth Edition, McGraw-Hill Health Professions Division, New York, 1996, pages 1225-1287. See also U.S. Pat. Nos. 3,387,001; 3,808,297; 3,894,000; 3,991,045; 4,012,390; 4,057,548; 4,086,417; 4,144,237; 4,150,146; 4,210,584; 4,215,062; 4,250,189; 4,258,052; 4,259,242; 4,296,105; 4,299,778; 4,367,239; 4,374,414; 4,375,432; 4,472,379; 4,588,831; 4,639,456; 4,767,855; 4,828,831; 4,841,045; 4,841,085; 4,908,356; 4,923,876; 5,030,620; 5,034,320; 5,047,528; 5,066,658; 5,166,149; 5,190,929; 5,215,738; 5,292,731; 5,380,897; 5,382,582; 5,409,915; 5,440,056; 5,446,139; 5,472,956; 5,527,905; 5,552,156; 5,594,158; 5,602,140; 5,665,768; 5,843,903; 6,080,874; 6,096,923; and RE030561.

In another embodiment, the cell-cycle inhibitor is camptothecin, mitoxantrone, etoposide, 5-fluorouracil, doxorubicin, methotrexate, peloruside A, mitomycin C, or a CDK-2 inhibitor or an analogue or derivative of any member of the class of listed compounds.

In another embodiment, the cell-cycle inhibitor is HTI-286, plicamycin; or mithramycin, or an analogue or derivative thereof.

Other examples of cell cycle inhibitors also include, e.g., 7-hexanoyltaxol (QP-2), cytochalasin A, lantrunculin D, actinomycin-D, Ro-31-7453 (3-(6-nitro-1-methyl-3-indolyl)-4-(1-methyl-3-indolyl)pyrrole-2,5-dione), PNU-151807, brostallicin, C2-ceramide, cytarabine ocfosfate (2(1H)-pyrimidinone, 4-amino-1-(5-O-(hydroxy(octadecyloxy)phosphinyl)-β-D-arabinofuranosyl)-, monosodium salt), paclitaxel (5β,20-epoxy-1,2 alpha,4,7β,10β,13 alpha-hexahydroxytax-11-en-9-one-4,10-diacetate-2-benzoate-13-(alpha-phenylhippurate)), doxorubicin (5,12-naphthacenedione, 10-((3-amino-2,3,6-trideoxy-alpha-L-lyxo-hexopyranosyl)oxy)-7,8,9,10-tetrahydro-6,8,11-trihydroxy-8-(hydroxyacetyl)-1-methoxy-, (8S)-cis-), daunorubicin (5,12-naphthacenedione, 8-acetyl-10-((3-amino-2,3,6-trideoxy-alpha-L-lyxo-hexopyranosyl)oxy)-7,8,9,10-tetrahydro-6,8,11-trihydroxy-1-methoxy-, (8S-cis)-), gemcitabine hydrochloride (cytidine, 2′-deoxy-2′,2′-difluoro-,monohydrochloride), nitacrine (1,3-propanediamine, N,N-dimethyl-N′-(1-nitro-9-acridinyl)-), carboplatin (platinum, diammine(1,1-cyclobutanedicarboxylato(2-))-, (SP-4-2)-), altretamine (1,3,5-triazine-2,4,6-triamine, N,N,N′,N′,N″,N″-hexamethyl-), teniposide (furo(3′,4′:6,7)naphtho(2,3-d)-1,3-dioxol-6(5a H)-one, 5,8,8a,9-tetrahydro-5-(4-hydroxy-3,5-dimethoxyphenyl)-9-((4,6-O-(2-thienylmethylene)-β-D-glucopyranosyl)oxy)-, (5R-(5alpha,5aβ,8aAlpha,9β(R*)))-), eptaplatin (platinum, ((4R,5R)-2-(1-methylethyl)-1,3-dioxolane-4,5-dimethanamine-kappa N4,kappa N5)(propanedioato(2-)-kappa O1, kappa O3)-, (SP-4-2)-), amrubicin hydrochloride (5,12-naphthacenedione, 9-acetyl-9-amino-7-((2-deoxy-β-D-erythro-pentopyranosyl)oxy)-7,8,9,10-tetrahydro-6,11-dihydroxy-, hydrochloride, (7S-cis)-), ifosfamide (2H-1,3,2-oxazaphosphorin-2-amine, N,3-bis(2-chloroethyl)tetrahydro-2-oxide), cladribine (adenosine, 2-chloro-2′-deoxy-), mitobronitol (D-mannitol, 1,6-dibromo-1,6-dideoxy-), fludaribine phosphate (9H-purin-6-amine, 2-fluoro-9-(5-O-phosphono-β-D-arabinofuranosyl)-), enocitabine (docosanamide, N-(1-β-D-arabinofuranosyl-1,2-dihydro-2-oxo-4-pyrimidinyl)-), vindesine (vincaleukoblastine, 3-(aminocarbonyl)-O4-deacetyl-3-de(methoxycarbonyl)-), idarubicin (5,12-naphthacenedione, 9-acetyl-7-((3-amino-2,3,6-trideoxy-alpha-L-lyxo-hexopyranosyl)oxy)-7,8,9,10-tetrahydro-6,9,11-trihydroxy-, (7S-cis)-), zinostatin (neocarzinostatin), vincristine (vincaleukoblastine, 22-oxo-), tegafur (2,4(1H,3H)-pyrimidinedione, 5-fluoro-1-(tetrahydro-2-furanyl)-), razoxane (2,6-piperazinedione, 4,4′-(1-methyl-1,2-ethanediyl)bis-), methotrexate (L-glutamic acid, N-(4-(((2,4-diamino-6-pteridinyl)methyl)methylamino)benzoyl)-), raltitrexed (L-glutamic acid, N-((5-(((1,4-dihydro-2-methyl-4-oxo-6-quinazolinyl)methyl)methylamino)-2-thienyl)carbonyl)-), oxaliplatin (platinum, (1,2-cyclohexanediamine-N,N′)(ethanedioato(2-)-O,O′)-, (SP-4-2-(1R-trans))-), doxifluridine (uridine, 5′-deoxy-5-fluoro-), mitolactol (galactitol, 1,6-dibromo-1,6-dideoxy-), piraubicin (5,12-naphthacenedione, 10-((3-amino-2,3,6-trideoxy-4-O-(tetrahydro-2H-pyran-2-yl)-alpha-L-lyxo-hexopyranosyl)oxy)-7,8,9,10-tetrahydro-6,8,11-trihydroxy-8-(hydroxyacetyl)-1-methoxy-, (8S-(8 alpha, 10 alpha(S*)))-), docetaxel ((2R,3S)-N-carboxy-3-phenylisoserine, N-tert-butyl ester, 13-ester with 5β,20-epoxy-1,2 alpha,4,7β,10β,13 alpha-hexahydroxytax-11-en-9-one 4-acetate 2-benzoate-), capecitabine (cytidine, 5-deoxy-5-fluoro-N-((pentyloxy)carbonyl)-), cytarabine (2(1H)-pyrimidone, 4-amino-1-β-D-arabino furanosyl-), valrubicin (pentanoic acid, 2-(1,2,3,4,6,11-hexahydro-2,5,12-trihydroxy-7-methoxy-6,11-dioxo-4-((2,3,6-trideoxy-3-((trifluoroacetyl)amino)-alpha-L-lyxo-hexopyranosyl)oxy)-2-naphthacenyl)-2-oxoethyl ester (2S-cis)-), trofosfamide (3-2-(chloroethyl)-2-(bis(2-chloroethyl)amino)tetrahydro-2H-1,3,2-oxazaphosphorin 2-oxide), prednimustine (pregna-1,4-diene-3,20-dione, 21-(4-(4-(bis(2-chloroethyl)amino)phenyl)-1-oxobutoxy)-11,17-dihydroxy-, (11β)-), lomustine (Urea, N-(2-chloroethyl)-N′-cyclohexyl-N-nitroso-), epirubicin (5,12-naphthacenedione, 10-((3-amino-2,3,6-trideoxy-alpha-L-arabino-hexopyranosyl)oxy)-7,8,9,10-tetrahydro-6,8,11-trihydroxy-8-(hydroxyacetyl)-1-methoxy-, (8S-cis)-), or an analogue or derivative thereof).

5) Cyclin Dependent Protein Kinase Inhibitors

In another embodiment, the pharmacologically active compound is a cyclin dependent protein kinase inhibitor (e.g., R-roscovitine, CYC-101, CYC-103, CYC-400, MX-7065, alvocidib (4H-1-Benzopyran-4-one, 2-(2-chlorophenyl)-5,7-dihydroxy-8-(3-hydroxy-1-methyl-4-piperidinyl)-, cis-(−)-), SU-9516, AG-12275, PD-0166285, CGP-79807, fascaplysin, GW-8510 (benzenesulfonamide, 4-(((Z)-(6,7-dihydro-7-oxo-8H-pyrrolo(2,3-g)benzothiazol-8-ylidene)methyl)amino)-N-(3-hydroxy-2,2-dimethylpropyl)-), GW-491619, Indirubin 3′ monoxime, GW8510, AZD-5438, ZK-CDK or an analogue or derivative thereof).

6) EGF (Epidermal Growth Factor) Receptor Kinase Inhibitors

In another embodiment, the pharmacologically active compound is an EGF (epidermal growth factor) kinase inhibitor (e.g., erlotinib (4-quinazolinamine, N-(3-ethynylphenyl)-6,7-bis(2-methoxyethoxy)-, monohydrochloride), erbstatin, BIBX-1382, gefitinib (4-quinazolinamine, N-(3-chloro-4-fluorophenyl)-7-methoxy-6-(3-(4-morpholinyl)propoxy)), or an analogue or derivative thereof).

7) Elastase Inhibitors

In another embodiment, the pharmacologically active compound is an elastase inhibitor (e.g., ONO-6818, sivelestat sodium hydrate (glycine, N-(2-(((4-(2,2-dimethyl-1-oxopropoxy)phenyl)sulfonyl)amino)benzoyl)-), erdosteine (acetic acid, ((2-oxo-2-((tetrahydro-2-oxo-3-thienyl)amino)ethyl)thio)-), MDL-100948A, MDL-104238 (N-(4-(4-morpholinylcarbonyl)benzoyl)-L-valyl-N′-(3,3,4,4,4-pentafluoro-1-(1-methylethyl)-2-oxobutyl)-L-2-azetamide), MDL-27324 (L-prolinamide, N-((5-(dimethylamino)-1-naphthalenyl)sulfonyl)-L-alanyl-L-alanyl-N-(3,3,3-trifluoro-1-(1-methylethyl)-2-oxopropyl)-, (S)-), SR-26831 (thieno(3,2-c)pyridinium, 5-((2-chlorophenyl)methyl)-2-(2,2-dimethyl-1-oxopropoxy)-4,5,6,7-tetrahydro-5-hydroxy-), Win-68794, Win-63110, SSR-69071 (2-(9(2-piperidinoethoxy)-4-oxo-4H-pyrido(1,2-a)pyrimidin-2-yloxymethyl)-4-(1-methylethyl)-6-methyoxy-1,2-benzisothiazol-3(2H)-one-1,1-dioxide), (N(Alpha)-(1-adamantylsulfonyl)N(epsilon)-succinyl-L-lysyl-L-prolyl-L-valinal), Ro-31-3537 (N alpha-(1-adamantanesulphonyl)-N-(4-carboxybenzoyl)-L-lysyl-alanyl-L-valinal), R-665, FCE-28204, ((6R,7R)-2-(benzoyloxy)-7-methoxy-3-methyl-4-pivaloyl-3-cephem 1,1-dioxide), 1,2-benzisothiazol-3(2H)-one, 2-(2,4-dinitrophenyl)-, 1,1-dioxide, L-658758 (L-proline, 1-((3-((acetyloxy)methyl)-7-methoxy-8-oxo-5-thia-1-azabicyclo(4.2.0)oct-2-en-2-yl)carbonyl)-, S,S-dioxide, (6R-cis)-), L-659286 (pyrrolidine, 1-((7-methoxy-8-oxo-3-(((1,2, 5,6-tetrahydro-2-methyl-5,6-dioxo-1,2,4-triazin-3-yl)thio)methyl)-5-thia-1-azabicyclo(4.2.0)oct-2-en-2-yl)carbonyl)-, S,S-dioxide, (6R-cis)-), L-680833 (benzeneacetic acid, 4-((3,3-diethyl-1-(((1-(4-methylphenyl)butyl)amino)carbonyl)-4-oxo-2-azetidinyl)oxy)-, (S-(R*,S*))-), FK-706 (L-prolinamide, N-[4-[[(carboxymethyl)amino]carbonyl]benzoyl]-L-valyl-N-[3,3,3-trifluoro-1-(1-methylethyl)-2-oxopropyl]-, monosodium salt), Roche R-665, or an analogue or derivative thereof).

8) Factor Xa Inhibitors

In another embodiment, the pharmacologically active compound is a factor Xa inhibitor (e.g., CY-222, fondaparinux sodium (alpha-D-glucopyranoside, methyl O-2-deoxy-6-O-sulfo-2-(sulfoamino)-alpha-D-glucopyranosyl-(1-4)-O-β-D-glucopyranuronosyl-(1-4)-O-2-deoxy-3,6-di-O-sulfo-2-(sulfoamino)-alpha-D-glucopyranosyl-(1-4)-O-2-O-sulfo-alpha-L-idopyranuronosyl-(1-4)-2-deoxy-2-(sulfoamino)-, 6-(hydrogen sulfate)), danaparoid sodium, or an analogue or derivative thereof).

9) Farnesyltransferase Inhibitors

In another embodiment, the pharmacologically active compound is a farnesyltransferase inhibitor (e.g., dichlorobenzoprim (2,4-diamino-5-(4-(3,4-dichlorobenzylamino)-3-nitrophenyl)-6-ethylpyrimidine), B-581, B-956 (N-(8(R)-amino-2(S)-benzyl-5(S)-isopropyl-9-sulfanyl-3(Z),6(E)-nonadienoyl)-L-methionine), OSI-754, perillyl alcohol (1-cyclohexene-1-methanol, 4-(1-methylethenyl)-, RPR-114334, Ionafarnib (1-piperidinecarboxamide, 4-(2-(4-((11R)-3,10-dibromo-8-chloro-6,11-dihydro-5H-benzo(5,6)cyclohepta(1,2-b)pyridin-11-yl)-1-piperidinyl)-2-oxoethyl)-), Sch-48755, Sch-226374, (7,8-dichloro-5H-dibenzo(b,e)(1,4)diazepin-11-yl)-pyridin-3-ylmethylamine, J-104126, L-639749, L-731734 (pentanamide, 2-((2-((2-amino-3-mercaptopropyl)amino)-3-methylpentyl)amino)-3-methyl-N-(tetrahydro-2-oxo-3-furanyl)-, (3S-(3R*(2R*(2R*(S*),3S*),3R*)))-), L-744832 (butanoic acid, 2-((2-((2-((2-amino-3-mercaptopropyl)amino)-3-methylpentyl)oxy)-1-oxo-3-phenylpropyl)amino)-4-(methylsulfonyl)-, 1-methylethyl ester, (2S-(1(R*(R*)),2R*(S*),3R*))-), L-745631 (1-piperazinepropanethiol, 1-amino-2-(2-methoxyethyl)-4-(1-naphthalenylcarbonyl)-, (βR,2S)-), N-acetyl-N-naphthylmethyl-2(S)-((1-(4-cyanobenzyl)-1H-imidazol-5-yl)acetyl)amino-3(S)-methylpentamine, (2alpha)-2-hydroxy-24,25-dihydroxylanost-8-en-3-one, BMS-316810, UCF-1-C (2,4-decadienamide, N-(5-hydroxy-5-(7-((2-hydroxy-5-oxo-1-cyclopenten-1-yl)amino-oxo-1,3,5-heptatrienyl)-2-oxo-7-oxabicyclo(4.1.0)hept-3-en-3-yl)-2,4,6-trimethyl-, (1S-(1alpha,3(2E,4E,6S*),5 alpha, 5(1E,3E,5E), 6 alpha))-), UCF-116-B, ARGLABIN (3H-oxireno[8,8a]azuleno[4,5-b]furan-8(4aH)-one, 5,6,6a,7,9a,9b-hexahydro-1,4a-dimethyl-7-methylene-, (3aR,4aS,6aS,9aS,9bR)-) from ARGLABIN—Paracure, Inc. (Virginia Beach, Va.), or an analogue or derivative thereof).

10) Fibrinogen Antagonists

In another embodiment, the pharmacologically active compound is a fibrinogen antagonist (e.g., 2(S)-((p-toluenesulfonyl)amino)-3-(((5,6,7,8,-tetrahydro-4-oxo-5-(2-(piperid in-4-yl)ethyl)-4H-pyrazolo-(1,5-a)(1,4)diazepin-2-yl)carbonyl)-amino)propionic acid, streptokinase (kinase (enzyme-activating), strepto-), urokinase (kinase (enzyme-activating), uro-), plasminogen activator, pamiteplase, monteplase, heberkinase, anistreplase, alteplase, pro-urokinase, picotamide (1,3-benzenedicarboxamide, 4-methoxy-N,N′-bis(3-pyridinylmethyl)-), or an analogue or derivative thereof).

11) Guanylate Cyclase Stimulants

In another embodiment, the pharmacologically active compound is a guanylate cyclase stimulant (e.g., isosorbide-5-mononitrate (D-glucitol, 1,4:3,6-dianhydro-, 5-nitrate), or an analogue or derivative thereof).

12) Heat Shock Protein 90 Antagonists

In another embodiment, the pharmacologically active compound is a heat shock protein 90 antagonist (e.g., geldanamycin; NSC-33050 (17-allylaminogeldanamycin), rifabutin (rifamycin XIV, 1′,4-didehydro-1-deoxy-1,4-dihydro-5′-(2-methylpropyl)-1-oxo-), 17MG, or an analogue or derivative thereof).

13) HMGCoA Reductase Inhibitors

In another embodiment, the pharmacologically active compound is an HMGCoA reductase inhibitor (e.g., BCP-671, BB-476, fluvastatin (6-heptenoic acid, 7-(3-(4-fluorophenyl)-1-(1-methylethyl)-1H-indol-2-yl)-3,5-dihydroxy-, monosodium salt, (R*,S*-(E))-(±)-), dalvastatin (2H-pyran-2-one, 6-(2-(2-(2-(4-fluoro-3-methylphenyl)-4,4,6,6-tetramethyl-1-cyclohexen-1-yl)ethenyl)tetrahydro)-4-hydroxy-, (4alpha,6β(E))-(+/−)-), glenvastatin (2H-pyran-2-one, 6-(2-(4-(4-fluorophenyl)-2-(1-methylethyl)-6-phenyl-3-pyridinyl)ethenyl)tetrahydro-4-hydroxy-, (4R-(4alpha,6β(E)))-), S-2468, N-(1-oxododecyl)-4Alpha, 10-dimethyl-8-aza-trans-decal-3β-ol, atorvastatin calcium (1H-Pyrrole-1-heptanoic acid, 2-(4-fluorophenyl)-β,delta-dihydroxy-5-(1-methylethyl)-3-phenyl-4-((phenylamino)carbonyl)-, calcium salt (R-(R*,R*))-), CP-83101 (6,8-nonadienoic acid, 3,5-dihydroxy-9,9-diphenyl-, methyl ester, (R*,S*-(E))-(+/−)-), pravastatin (1-naphthaleneheptanoic acid, 1,2,6,7,8,8a-hexahydro-β,delta,6-trihydroxy-2-methyl-8-(2-methyl-1-oxobutoxy)-, monosodium salt, (1S-(1 alpha(βS*,deltaS*),2 alpha,6 alpha,8β(R*),8a alpha))-), U-20685, pitavastatin (6-heptenoic acid, 7-(2-cyclopropyl-4-(4-fluorophenyl)-3-quinolinyl)-3,5-dihydroxy-, calcium salt (2:1), (S-(R*,S*-(E)))-), N-((1-methylpropyl)carbonyl)-8-(2-(tetrahydro-4-hydroxy-6-oxo-2H-pyran-2-yl)ethyl)-perhydro-isoquinoline, dihydromevinolin (butanoic acid, 2-methyl-, 1,2,3,4,4a,7,8,8a-octahydro-3,7-dimethyl-8-(2-(tetrahydro-4-hydroxy-6-oxo-2H-pyran-2-yl)ethyl)-1-naphthalenyl ester(1 alpha(R*), 3 alpha, 4a alpha,7β,8β(2S*,4S*),8aβ))-), HBS-107, dihydromevinolin (butanoic acid, 2-methyl-, 1,2,3,4,4a,7,8,8a-octahydro-3,7-dimethyl-8-(2-(tetrahydro-4-hydroxy-6-oxo-2H-pyran-2-yl)ethyl)-1-naphthalenyl ester(1 alpha(R*), 3 alpha,4a alpha,7β,8β(2S*,4S*),8aβ))-), L-669262 (butanoic acid, 2,2-dimethyl-, 1,2,6,7,8,8a-hexahydro-3,7-dimethyl-6-oxo-8-(2-(tetrahydro-4-hydroxy-6-oxo-2H-pyran-2-yl)ethyl)-1-naphthalenyl(1S-(1Alpha,7β,8β(2S*,4S*),8aβ))-), simvastatin (butanoic acid, 2,2-dimethyl-, 1,2,3,7,8,8a-hexahydro-3,7-dimethyl-8-(2-(tetrahydro-4-hydroxy-6-oxo-2H-pyran-2-yl)ethyl)-1-naphthalenyl ester, (1S-(1 alpha, 3alpha,7β,8β(2S*,4S*),8aβ))-), rosuvastatin calcium (6-heptenoic acid, 7-(4-(4-fluorophenyl)-6-(1-methylethyl)-2-(methyl(methylsulfonyl)amino)-5-pyrimd inyl)-3,5-dihydroxy-calcium salt (2:1) (S-(R*,S*-(E)))), meglutol (2-hydroxy-2-methyl-1,3-propandicarboxylic acid), lovastatin (butanoic acid, 2-methyl-, 1,2,3,7,8,8a-hexahydro-3,7-dimethyl-8-(2-(tetrahydro-4-hydroxy-6-oxo-2H-pyran-2-yl)ethyl)-1-naphthalenyl ester, (1S-(1 alpha.(R*),3 alpha,7β,8β(2S*,4S*),8aβ))-), or an analogue or derivative thereof).

14) Hydroorotate Dehydrogenase Inhibitors

In another embodiment, the pharmacologically active compound is a hydroorotate dehydrogenase inhibitor (e.g., leflunomide (4-isoxazolecarboxamide, 5-methyl-N-(4-(trifluoromethyl)phenyl)-), laflunimus (2-propenamide, 2-cyano-3-cyclopropyl-3-hydroxy-N-(3-methyl-4(trifluoromethyl)phenyl)-, (Z)-), or atovaquone (1,4-naphthalenedione, 2-[4-(4-chlorophenyl)cyclohexyl]-3-hydroxy-, trans-, or an analogue or derivative thereof).

15) IKK2 Inhibitors

In another embodiment, the pharmacologically active compound is an IKK2 inhibitor (e.g., MLN-120B, SPC-839, or an analogue or derivative thereof).

16) IL-1, ICE and IRAK Antagonists

In another embodiment, the pharmacologically active compound is an IL-1, ICE or an IRAK antagonist (e.g., E-5090 (2-propenoic acid, 3-(5-ethyl-4-hydroxy-3-methoxy-1-naphthalenyl)-2-methyl-, (Z)-), CH-164, CH-172, CH-490, AMG-719, iguratimod (N-(3-(formylamino)-4-oxo-6-phenoxy-4H-chromen-7-yl) methanesulfonamide), AV94-88, pralnacasan (6H-pyridazino(1,2-a)(1,2)diazepine-1-carboxamide, N-((2R,3S)-2-ethoxytetrahydro-5-oxo-3-furanyl)octahydro-9-((1-isoquinolinylcarbonyl)amino)-6,10-dioxo-, (1S,9S)-), (2S-cis)-5-(benzyloxycarbonylamino-1,2,4,5,6,7-hexahydro-4-(oxoazepino(3,2,1-hi)indole-2-carbonyl)-amino)-4-oxobutanoic acid, AVE-9488, esonarimod (benzenebutanoic acid, alpha-((acetylthio)methyl)-4-methyl-gamma-oxo-), pralnacasan (6H-pyridazino(1,2-a)(1,2)diazepine-1-carboxamide, N-((2R,3S)-2-ethoxytetrahydro-5-oxo-3-furanyl)octahydro-9-((1-isoquinolinylcarbonyl)amino)-6,10-dioxo-, (1S,9S)-), tranexamic acid (cyclohexanecarboxylic acid, 4-(aminomethyl)-, trans-), Win-72052, romazarit (Ro-31-3948) (propanoic acid, 2-((2-(4-chlorophenyl)-4-methyl-5-oxazolyl)methoxy)-2-methyl-), PD-163594, SDZ-224-015 (L-alaninamide N-((phenylmethoxy)carbonyl)-L-valyl-N-((1S)-3-((2,6-dichlorobenzoyl)oxy)-1-(2-ethoxy-2-oxoethyl)-2-oxopropyl)-); L-709049 (L-alaninamide, N-acetyl-L-tyrosyl-L-valyl-N-(2-carboxy-1-formylethyl)-, (S)-), TA-383 (1H-imidazole, 2-(4-chlorophenyl)-4,5-dihydro-4,5-diphenyl-, monohydrochloride, cis-), EI-1507-1 (6a,12a-epoxybenz(a)anthracen-1,12(2H, 7H)-dione, 3,4-dihydro-3,7-dihydroxy-8-methoxy-3-methyl-), ethyl 4-(3,4-dimethoxyphenyl)-6,7-dimethoxy-2-(1,2,4-triazol-1-yl methyl)quinoline-3-carboxylate, EI-1941-1, TJ-114, anakinra (interleukin 1 receptor antagonist (human isoform x reduced), N2-L-methionyl-), IX-207-887 (acetic acid, (10-methoxy-4H-benzo[4,5]cyclohepta[1,2-b]thien-4-ylidene)-), K-832, or an analogue or derivative thereof).

17) IL-4 Agonists

In another embodiment, the pharmacologically active compound is an IL-4 agonist (e.g., glatiramir acetate (L-glutamic acid, polymer with L-alanine, L-lysine and L-tyrosine, acetate (salt)), or an analogue or derivative thereof).

18) Immunomodulatory Agents

In another embodiment, the pharmacologically active compound is an immunomodulatory agent (e.g., biolimus, ABT-578, methylsulfamic acid 3-(2-methoxyphenoxy)-2-(((methylamino)sulfonyl)oxy)propyl ester, sirolimus (also referred to as rapamycin or RAPAMUNE (American Home Products, Inc., Madison, N.J.)), CCl-779 (rapamycin 42-(3-hydroxy-2-(hydroxymethyl)-2-methylpropanoate)), LF-15-0195, NPC15669 (L-leucine, N-(((2,7-dimethyl-9H-fluoren-9-yl)methoxy)carbonyl)-), NPC-15670 (L-leucine, N-(((4,5-dimethyl-9H-fluoren-9-yl)methoxy)carbonyl)-), N PC-16570 (4-(2-(fluoren-9-yl)ethyloxy-carbonyl)aminobenzoic acid), sufosfamide (ethanol, 2-((3-(2-chloroethyl)tetrahydro-2H-1,3,2-oxazaphosphorin-2-yl)amino)-, methanesulfonate (ester), P-oxide), tresperimus (2-(N-(4-(3-aminopropylamino)butyl)carbamoyloxy)-N-(6-guanidinohexyl)acetamide), 4-(2-(fluoren-9-yl)ethoxycarbonylamino)-benzo-hydroxamic acid, iaquinimod, PBI-1411, azathioprine (6-((1-Methyl-4-nitro-1H-imidazol-5-yl)thio)-1H-purine), PBI0032, beclometasone, MDL-28842 (9H-purin-6-amine, 9-(5-deoxy-5-fluoro-β-D-threo-pent-4-enofuranosyl)-, (Z)-), FK-788, AVE-1726, ZK-90695, ZK-90695, Ro-54864, didemnin-B, Illinois (didemnin A, N-(1-(2-hydroxy-1-oxopropyl)-L-prolyl)-, (S)-), SDZ-62-826 (ethanaminium, 2-((hydroxy((1-((octadecyloxy)carbonyl)-3-piperidinyl)methoxy)phosphinyl)oxy)-N,N, N-trimethyl-, inner salt), argyrin B ((4S,7S,13R,22R)-13-Ethyl-4-(1H-indol-3-ylmethyl)-7-(4-methoxy-1H-indol-3-ylmethyl)18,22-dimethyl-16-methyl-ene-24-thia-3,6,9,12,15,18,21,26-octaazabicyclo(21.2.1)-hexacosa-1 (25),23(26)-diene-2,5,8,11,14,17,20-heptaone), everolimus (rapamycin, 42-O-(2-hydroxyethyl)-), SAR-943, L-687795, 6-((4-chlorophenyl)sulfinyl)-2,3-dihydro-2-(4-methoxy-phenyl)-5-methyl-3-oxo-4-pyridazinecarbonitrile, 91 Y78 (1H-imidazo(4,5-c)pyridin-4-amine, 1-β-D-ribofuranosyl-), auranofin (gold, (1-thio-β-D-glucopyranose 2,3,4,6-tetraacetato-S)(triethylphosphine)-), 27-0-demethylrapamycin, tipredane (androsta-1,4-dien-3-one, 17-(ethylthio)-9-fluoro-11-hydroxy-17-(methylthio)-, (11β,17 alpha)-), AI-402, LY-178002 (4-thiazolidinone, 5-((3,5-bis(1,1-dimethylethyl)-4-hydroxyphenyl)methylene)-), SM-8849 (2-thiazolamine, 4-(1-(2-fluoro(1,1′-biphenyl)-4-yl)ethyl)-N-methyl-), piceatannol, resveratrol, triamcinolone acetonide (pregna-1,4-diene-3,20-dione, 9-fluoro-11,21-dihydroxy-16,17-((1-methylethylidene)bis(oxy))-, (11β,16 alpha)-), ciclosporin (cyclosporin A), tacrolimus (15,19-epoxy-3H-pyrido(2,1-c)(1,4)oxaazacyclotricosine-1,7,20,21 (4H,23H)-tetrone, 5,6,8,11,12,13,14,15,16,17,18,19,24,25,26,26a-hexadecahydro-5,19-dihydroxy-3-(2-(4-hydroxy-3-methoxycyclohexyl)-1-methylethenyl)-14,16-dimethoxy-4,10,12,18-tetramethyl-8-(2-propenyl)-, (3S-(3R*(E(1 S*,3S*,4S*)),4S*,5R*,8S*,9E,12R*,14R*,15S*,16R*,18S*,19S*,26aR*))-), gusperimus (heptanamide, 7-((aminoiminomethyl)amino)-N-(2-((4-((3-aminopropyl)amino)butyl)amino)-1-hydroxy-2-oxoethyl)-, (+/−)-), tixocortol pivalate (pregn-4-ene-3,20-dione, 21-((2,2-dimethyl-1-oxopropyl)thio)-11,17-dihydroxy-, (11β)-), alefacept (1-92 LFA-3 (antigen) (human) fusion protein with immunoglobulin G1 (human hinge-CH2-CH3 gamma1-chain), dimer), halobetasol propionate (pregna-1,4-diene-3,20-dione, 21-chloro-6,9-difluoro-11-hydroxy-16-methyl-17-(1-oxopropoxy)-, (6Alpha,11β,16)-), iloprost trometamol (pentanoic acid, 5-(hexahydro-5-hydroxy-4-(3-hydroxy-4-methyl-1-octen-6-ynyl)-2(1H)-pentalenylidene)-), beraprost (1H-cyclopenta(b)benzofuran-5-butanoic acid, 2,3,3a,8b-tetrahydro-2-hydroxy-1-(3-hydroxy-4-methyl-1-octen-6-ynyl)-), rimexolone (androsta-1,4-dien-3-one, 11-hydroxy-16,17-dimethyl-17-(1-oxopropyl)-, (11β,16Alpha,17β)-), dexamethasone (pregna-1,4-diene-3,20-dione,9-fluoro-11,17,21-trihydroxy-16-methyl-, (11β,16alpha)-), sulindac (cis-5-fluoro-2-methyl-1-((p-methylsulfinyl)benzylidene)indene-3-acetic acid), proglumetacin (1H-Indole-3-acetic acid, 1-(4-chlorobenzoyl)-5-methoxy-2-methyl-, 2-(4-(3-((4-(benzoylamino)-5-(dipropylamino)-1,5-dioxopentyl)oxy)propyl)-1-piperazinyl)ethylester, (+/−)-), alclometasone dipropionate (pregna-1,4-diene-3,20-dione, 7-chloro-11-hydroxy-16-methyl-17,21-bis(1-oxopropoxy)-, (7alpha, 11β,16alpha)-), pimecrolimus (15,19-epoxy-3H-pyrido(2,1-c)(1,4)oxaazacyclotricosine-1,7,20,21(4H,23H)-tetrone, 3-(2-(4-chloro-3-methoxycyclohexyl)-1-methyletheny)-8-ethyl-5,6,8,11,12,13,14,15,16,17,18,19,24,25,26,26a-hexadecahydro-5,19-dihydroxy-14,16-dimethoxy-4,10,12,18-tetramethyl-, (3S-(3R*(E(1S*,3S*,4R*)),4S*,5R*,8S*,9E,12R*,14R*,15S*,16R*,18S*,19S*,26aR*))-), hydrocortisone-17-butyrate (pregn-4-ene-3,20-dione, 11,21-dihydroxy-17-(1-oxobutoxy)-, (11β)-), mitoxantrone (9,10-anthracenedione, 1,4-dihydroxy-5,8-bis((2-((2-hydroxyethyl)amino)ethyl)amino)-), mizoribine (1H-imidazole-4-carboxamide, 5-hydroxy-1-β-D-ribofuranosyl-), prednicarbate (pregna-1,4-diene-3,20-dione, 17-((ethoxycarbonyl)oxy)-11-hydroxy-21-(1-oxopropoxy)-, (11β)-), iobenzarit (benzoic acid, 2-((2-carboxyphenyl)amino)-4-chloro-), glucametacin (D-glucose, 2-(((1-(4-chlorobenzoyl)-5-methoxy-2-methyl-1H-indol-3-yl)acetyl)amino)-2-deoxy-), fluocortolone monohydrate ((6 alpha)-fluoro-16alpha-methylpregna-1,4-dien-11β,21-diol-3,20-dione), fluocortin butyl (pregna-1,4-dien-21-oic acid, 6-fluoro-11-hydroxy-16-methyl-3,20-dioxo-, butyl ester, (6alpha, 11β,16alpha)-), difluprednate (pregna-1,4-diene-3,20-dione, 21-(acetyloxy)-6,9-difluoro-11-hydroxy-17-(1-oxobutoxy)-, (6 alpha,11β)-), diflorasone diacetate (pregna-1,4-diene-3,20-dione, 17,21-bis(acetyloxy)-6,9-difluoro-11-hydroxy-16-methyl-, (6Alpha,11β,16β)-), dexamethasone valerate (pregna-1,4-diene-3,20-dione, 9-fluoro-11,21-dihydroxy-16-methyl-17-((1-oxopentyl)oxy)-, (11β,16Alpha)-), methylprednisolone, deprodone propionate (pregna-1,4-diene-3,20-dione, 11-hydroxy-17-(1-oxopropoxy)-, (11.beta.)-), bucillamine (L-cysteine, N-(2-mercapto-2-methyl-1-oxopropyl)-), amcinonide (benzeneacetic acid, 2-amino-3-benzoyl-, monosodium salt, monohydrate), acemetacin (1H-indole-3-acetic acid, 1-(4-chlorobenzoyl)-5-methoxy-2-methyl-, carboxymethyl ester), or an analogue or derivative thereof).

Further, analogues of rapamycin include tacrolimus and derivatives thereof (e.g., EP0184162B1 and U.S. Pat. No. 6,258,823) everolimus and derivatives thereof (e.g., U.S. Pat. No. 5,665,772). Further representative examples of sirolimus analogues and derivatives can be found in PCT Publication Nos. WO 97/10502, WO 96/41807, WO 96/35423, WO 96/03430, WO 96/00282, WO 95/16691, WO 95/15328, WO 95/07468, WO 95/04738, WO 95/04060, WO 94/25022, WO 94/21644, WO 94/18207, WO 94/10843, WO 94/09010, WO 94/04540, WO 94/02485, WO 94/02137, WO 94/02136, WO 93/25533, WO 93/18043, WO 93/13663, WO 93/11130, WO 93/10122, WO 93/04680, WO 92/14737, and WO 92/05179. Representative U.S. patents include U.S. Pat. Nos. 6,342,507; 5,985,890; 5,604,234; 5,597,715; 5,583,139; 5,563,172; 5,561,228; 5,561,137; 5,541,193; 5,541,189; 5,534,632; 5,527,907; 5,484,799; 5,457,194; 5,457,182; 5,362,735; 5,324,644; 5,318,895; 5,310,903; 5,310,901; 5,258,389; 5,252,732; 5,247,076; 5,225,403; 5,221,625; 5,210,030; 5,208,241; 5,200,411; 5,198,421; 5,147,877; 5,140,018; 5,116,756; 5,109,112; 5,093,338; and 5,091,389.

The structures of sirolimus, everolimus, and tacrolimus are provided below:

| Name | Code Name | Company | Structure |
|---|---|---|---|
| Everolimus | SAR-943 | Novartis | See below |
| Sirolimus | AY-22989 | Wyeth | See below |
| RAPAMUNE | NSC-226080 | | |
| Rapamycin | | | |
| Tacrolimus | FK506 | Fujusawa | See below |

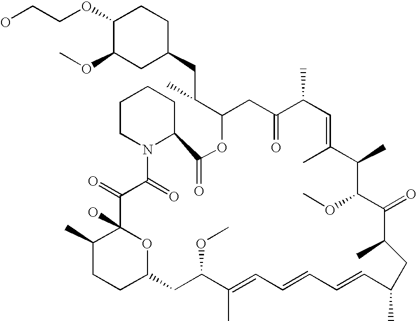

Everolimus

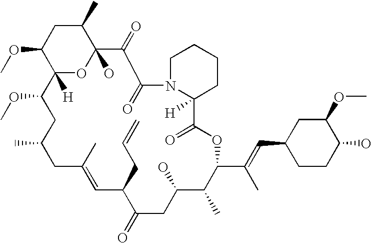

Tacrolimus

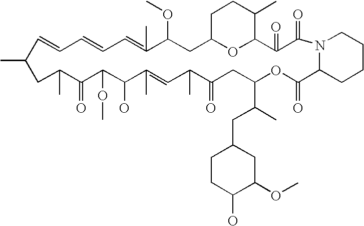

Sirolimus

Further sirolimus analogues and derivatives include tacrolimus and derivatives thereof (e.g., EP0184162B1 and U.S. Pat. No. 6,258,823) everolimus and derivatives thereof (e.g., U.S. Pat. No. 5,665,772). Further representative examples of sirolimus analogues and derivatives include ABT-578 and others may be found in PCT Publication Nos. WO 97/10502, WO 96/41807, WO 96/35423, WO 96/03430, WO 9600282, WO 95/16691, WO 9515328, WO 95/07468, WO 95/04738, WO 95/04060, WO 94/25022, WO 94/21644, WO 94/18207, WO 94/10843, WO 94/09010, WO 94/04540, WO 94/02485, WO 94/02137, WO 94/02136, WO 93/25533, WO 93/18043, WO 93/13663, WO 93/11130, WO 93/10122, WO 93/04680, WO 92/14737, and WO 92/05179. Representative U.S. patents include U.S. Pat. Nos. 6,342,507; 5,985,890; 5,604,234; 5,597,715; 5,583,139; 5,563,172; 5,561,228; 5,561,137; 5,541,193; 5,541,189; 5,534,632; 5,527,907; 5,484,799; 5,457,194; 5,457,182; 5,362,735; 5,324,644; 5,318,895; 5,310,903; 5,310,901; 5,258,389; 5,252,732; 5,247,076; 5,225,403; 5,221,625; 5,210,030; 5,208,241, 5,200,411; 5,198,421; 5,147,877; 5,140,018; 5,116,756; 5,109,112; 5,093,338; and 5,091,389.

In one aspect, the fibrosis-inhibiting agent may be, e.g., rapamycin (sirolimus), everolimus, biolimus, tresperimus, auranofin, 27-0-demethylrapamycin, tacrolimus, gusperimus, pimecrolimus, or ABT-578.

19) Inosine Monophosphate Dehydrogenase Inhibitors

In another embodiment, the pharmacologically active compound is an inosine monophosphate dehydrogenase (IMPDH) inhibitor (e.g., mycophenolic acid, mycophenolate mofetil (4-hexenoic acid, 6-(1,3-dihydro-4-hydroxy-6-methoxy-7-methyl-3-oxo-5-isobenzofuranyl)-4-methyl-, 2-(4-morpholinyl)ethyl ester, (E)-), ribavirin (1H-1,2,4-triazole-3-carboxamide, 1-β-D-ribofuranosyl-), tiazofurin (4-thiazolecarboxamide, 2-β-D-ribofuranosyl-), viramidine, aminothiadiazole, thiophenfurin, tiazofurin) or an analogue or derivative thereof. Additional representative examples are included in U.S. Pat. Nos. 5,536,747, 5,807,876, 5,932,600, 6,054,472, 6,128,582, 6,344,465, 6,395,763, 6,399,773, 6,420,403, 6,479,628, 6,498,178, 6,514,979, 6,518,291, 6,541,496, 6,596,747, 6,617,323, 6,624,184, Patent Application Publication Nos. 2002/0040022A1, 2002/0052513A1, 2002/0055483A1, 2002/0068346A1, 2002/0111378A1, 2002/0111495A1, 2002/0123520A1, 2002/0143176A1, 2002/0147160A1, 2002/0161038A1, 2002/0173491A1, 2002/0183315A1, 2002/0193612A1, 2003/0027845A1, 2003/0068302A1, 2003/0105073A1, 2003/0130254A1, 2003/0143197A1, 2003/0144300A1, 2003/0166201A1, 2003/0181497A1, 2003/0186974A1, 2003/0186989A1, 2003/0195202A1, and PCT Publication Nos. WO 0024725A1, WO 00/25780A1, WO 00/26197A1, WO 00/51615A1, WO 00/56331A1, WO 00/73288A1, WO 01/00622A1, WO 01/66706A1, WO 01/79246A2, WO 01/81340A2, WO 01/85952A2, WO 02/16382A1, WO 02/18369A2, WO 2051814A1, WO 2057287A2, WO2057425A2, WO 2060875A1, WO 2060896A1, WO 2060898A1, WO 2068058A2, WO 3020298A1, WO 3037349A1, WO 3039548A1, WO 3045901A2, WO 3047512A2, WO 3053958A1, WO 3055447A2, WO 3059269A2, WO 3063573A2, WO 3087071 A1, WO 90/01545A1, WO 97/40028A1, WO 97/41211A1, WO 98/40381A1, and WO 99/55663A1).

20) Leukotriene Inhibitors

In another embodiment, the pharmacologically active compound is a leukotreine inhibitor (e.g., ONO-4057(benzenepropanoic acid, 2-(4-carboxybutoxy)-6-((6-(4-methoxyphenyl)-5-hexenyl)oxy)-, (E)-), ONO-LB-448, pirodomast 1,8-naphthyridin-2(1H)-one, 4-hydroxy-1-phenyl-3-(1-pyrrolidinyl)-, Sch-40120 (benzo(b)(1,8)naphthyridin-5(7H)-one, 10-(3-chlorophenyl)-6,8,9,10-tetrahydro-), L-656224 (4-benzofuranol, 7-chloro-2-((4-methoxyphenyl)methyl)-3-methyl-5-propyl-), MAFP (methyl arachidonyl fluorophosphonate), ontazolast (2-benzoxazolamine, N-(2-cyclohexyl-1-(2-pyridinyl)ethyl)-5-methyl-, (S)-), amelubant (carbamic acid, ((4-((3-((4-(1-(4-hydroxyphenyl)-1-methylethyl)phenoxy)methyl)phenyl)methoxy)phenyl)iminomethyl)-ethyl ester), SB-201993 (benzoic acid, 3-((((6-((1E)-2-carboxyethenyl)-5-((8-(4-methoxyphenyl)octyl)oxy)-2-pyridinyl)methyl)thio)methyl)-), LY-203647 (ethanone, 1-(2-hydroxy-3-propyl-4-(4-(2-(4-(1H-tetrazol-5-yl)butyl)-2H-tetrazol-5-yl)butoxy)phenyl)-), LY-210073, LY-223982 (benzenepropanoic acid, 5-(3-carboxybenzoyl)-2-((6-(4-methoxyphenyl)-5-hexenyl)oxy)-, (E)-), LY-293111 (benzoic acid, 2-(3-(3-((5-ethyl-4′-fluoro-2-hydroxy(1,1′-biphenyl)-4-yl)oxy)propoxy)-2-propylphenoxy)-), SM-9064 (pyrrolidine, 1-(4,11-dihydroxy-13-(4-methoxyphenyl)-1-oxo-5,7,9-tridecatrienyl)-, (E,E,E)-), T-0757 (2,6-octadienamide, N-(4-hydroxy-3,5-dimethylphenyl)-3,7-dimethyl-, (2E)-), or an analogue or derivative thereof).

21) MCP-1 Antagonists

In another embodiment, the pharmacologically active compound is a MCP-1 antagonist (e.g., nitronaproxen (2-napthaleneacetic acid, 6-methoxy-alpha-methyl 4-(nitrooxy)butyl ester (alpha S)-), bindarit (2-(1-benzylindazol-3-ylmethoxy)-2-methylpropanoic acid), 1-alpha-25 dihydroxy vitamin D₃, or an analogue or derivative thereof).

22) MMP Inhibitors

In another embodiment, the pharmacologically active compound is a matrix metalloproteinase (MMP) inhibitor (e.g., D-9120, doxycycline (2-naphthacenecarboxamide, 4-(dimethylamino)-1,4,4a,5,5a,6,11,12a-octahydro-3,5,10,12,12a-pentahydroxy-6-methyl-1,11-dioxo-(4S-(4 alpha, 4a alpha, 5 lpha, 5a alpha, 6 alpha, 12a alpha))-), BB-2827, BB-1101 (2S-allyl-N-1-hydroxy-3R-isobutyl-N-4-(1S-methylcarbamoyl-2-phenylethyl)-succinamide), BB-2983, solimastat (N′-(2,2-dimethyl-1(S)-(N-(2-pyridyl)carbamoyl)propyl)-N-4-hydroxy-2(R)-isobutyl-3(S)-methoxysuccinamide), batimastat (butanediamide, N4-hydroxy-N1-(2-(methylamino)-2-oxo-1-(phenylmethyl)ethyl)-2-(2-methylpropyl)-3-((2-thienylthio)methyl)-, (2R-(1(S*),2R*,3S*))-), CH-138, CH-5902, D-1927, D-5410, EF-13 (gamma-linolenic acid lithium salt), CMT-3 (2-naphthacenecarboxamide, 1,4,4a,5,5a,6,11,12a-octahydro-3,10,12,12a-tetrahydroxy-1,11-dioxo-, (4aS,5a R,12aS)-), marimastat (N-(2,2-dimethyl-1(S)-(N-methylcarbamoyl)propyl)-N,3(S)-dihydroxy-2(R)-isobutylsuccinamide), TIMP'S, ONO-4817, rebimastat (L-Valinamide, N-((2S)-2-mercapto-1-oxo-4-(3,4,4-trimethyl-2,5-dioxo-1-imidazolidinyl)butyl)-L-leucyl-N,3-dimethyl-), PS-508, CH-715, nimesulide (methanesulfonamide, N-(4-nitro-2-phenoxyphenyl)-), hexahydro-2-(2(R)-(1(RS)-(hydroxycarbamoyl)-4-phenylbutyl)nonanoyl)-N-(2,2,6,6-etramethyl-4-piperidinyl)-3(S)-pyridazine carboxamide, Rs-113-080, Ro-1130830, cipemastat (1-piperid inebutanamide, β-(cyclopentylmethyl)-N-hydroxy-gamma-oxo-alpha-((3,4,4-trimethyl-2,5-dioxo-1-imidazolidinyl)methyl)-,(alpha R,βR)-), 5-(4′-biphenyl)-5-(N-(4-nitrophenyl)piperazinyl)barbituric acid, 6-methoxy-1,2,3,4-tetrahydro-norharman-1-carboxylic acid, Ro-31-4724 (L-alanine, N-(2-(2-(hydroxyamino)-2-oxoethyl)-4-methyl-1-oxopentyl)-L-leucyl-, ethyl ester), prinomastat (3-thiomorpholinecarboxamide, N-hydroxy-2,2-dimethyl-4-((4-(4-pyridinyloxy) phenyl)sulfonyl)-, (3R)-), AG-3433 (1H-pyrrole-3-propanic acid, 1-(4′-cyano(1,1′-biphenyl)-4-yl)-b-((((3S)-tetrahydro-4,4-dimethyl-2-oxo-3-furanyl)amino)carbonyl)-, phenylmethyl ester, (bS)-), PNU-142769 (2H-Isoindole-2-butanamide, 1,3-dihydro-N-hydroxy-alpha-((3S)-3-(2-methylpropyl)-2-oxo-1-(2-phenylethyl)-3-pyrrolidinyl)-1,3-dioxo-, (alpha R)-), (S)-1-(2-((((4,5-dihydro-5-thioxo-1,3,4-thiadiazol-2-yl)amino)-carbonyl)amino)-1-oxo-3-(pentafluorophenyl)propyl)-4-(2-pyridinyl)piperazine, SU-5402 (1H-pyrrole-3-propanoic acid, 2-((1,2-dihydro-2-oxo-3H-indol-3-ylidene)methyl)-4-methyl-), SC-77964, PNU-171829, CGS-27023A, N-hydroxy-2(R)-((4-methoxybenzene-sulfonyl)(4-picolyl)amino)-2-(2-tetrahydrofuranyl)-acetamide, L-758354 ((1,1′-biphenyl)-4-hexanoic acid, alpha-butyl-gamma-(((2,2-dimethyl-1-((methylamino)carbonyl)propyl)amino)carbonyl)-4′-fluoro-, (alpha S-(alpha R*,gammaS*(R*)))-, GI-155704A, CPA-926, TMI-005, XL-784, or an analogue or derivative thereof). Additional representative examples are included in U.S. Pat. Nos. 5,665,777; 5,985,911; 6,288,261; 5,952,320; 6,441,189; 6,235,786; 6,294,573; 6,294,539; 6,563,002; 6,071,903; 6,358,980; 5,852,213; 6,124,502; 6,160,132; 6,197,791; 6,172,057; 6,288,086; 6,342,508; 6,228,869; 5,977,408; 5,929,097; 6,498,167; 6,534,491; 6,548,524; 5,962,481; 6,197,795; 6,162,814; 6,441,023; 6,444,704; 6,462,073; 6,162,821; 6,444,639; 6,262,080; 6,486,193; 6,329,550; 6,544,980; 6,352,976; 5,968,795; 5,789,434; 5,932,763; 6,500,847; 5,925,637; 6,225,314; 5,804,581; 5,863,915; 5,859,047; 5,861,428; 5,886,043; 6,288,063; 5,939,583; 6,166,082; 5,874,473; 5,886,022; 5,932,577; 5,854,277; 5,886,024; 6,495,565; 6,642,255; 6,495,548; 6,479,502; 5,696,082; 5,700,838; 6,444,639; 6,262,080; 6,486,193; 6,329,550; 6,544,980; 6,352,976; 5,968,795; 5,789,434; 5,932,763; 6,500,847; 5,925,637; 6,225,314; 5,804,581; 5,863,915; 5,859,047; 5,861,428; 5,886,043; 6,288,063; 5,939,583; 6,166,082; 5,874,473; 5,886,022; 5,932,577; 5,854,277; 5,886,024; 6,495,565; 6,642,255; 6,495,548; 6,479,502; 5,696,082; 5,700,838; 5,861,436; 5,691,382; 5,763,621; 5,866,717; 5,902,791; 5,962,529; 6,017,889; 6,022,873; 6,022,898; 6,103,739; 6,127,427; 6,258,851; 6,310,084; 6,358,987; 5,872,152; 5,917,090; 6,124,329; 6,329,373; 6,344,457; 5,698,706; 5,872,146; 5,853,623; 6,624,144; 6,462,042; 5,981,491; 5,955,435; 6,090,840; 6,114,372; 6,566,384; 5,994,293; 6,063,786; 6,469,020; 6,118,001; 6,187,924; 6,310,088; 5,994,312; 6,180,611; 6,110,896; 6,380,253; 5,455,262; 5,470,834; 6,147,114; 6,333,324; 6,489,324; 6,362,183; 6,372,758; 6,448,250; 6,492,367; 6,380,258; 6,583,299; 5,239,078; 5,892,112; 5,773,438; 5,696,147; 6,066,662; 6,600,057; 5,990,158; 5,731,293; 6,277,876; 6,521,606; 6,168,807; 6,506,414; 6,620,813; 5,684,152; 6,451,791; 6,476,027; 6,013,649; 6,503,892; 6,420,427; 6,300,514; 6,403,644; 6,177,466; 6,569,899; 5,594,006; 6,417,229; 5,861,510; 6,156,798; 6,387,931; 6,350,907; 6,090,852; 6,458,822; 6,509,337; 6,147,061; 6,114,568; 6,118,016; 5,804,593; 5,847,153; 5,859,061; 6,194,451; 6,482,827; 6,638,952; 5,677,282; 6,365,630; 6,130,254; 6,455,569; 6,057,369; 6,576,628; 6,110,924; 6,472,396; 6,548,667; 5,618,844; 6,495,578; 6,627,411; 5,514,716; 5,256,657; 5,773,428; 6,037,472; 6,579,890; 5,932,595; 6,013,792; 6,420,415; 5,532,265; 5,691,381; 5,639,746; 5,672,598; 5,830,915; 6,630,516; 5,324,634; 6,277,061; 6,140,099; 6,455,570; 5,595,885; 6,093,398; 6,379,667; 5,641,636; 5,698,404; 6,448,058; 6,008,220; 6,265,432; 6,169,103; 6,133,304; 6,541,521; 6,624,196; 6,307,089; 6,239,288; 5,756,545; 6,020,366; 6,117,869; 6,294,674; 6,037,361; 6,399,612; 6,495,568; 6,624,177; 5,948,780; 6,620,835; 6,284,513; 5,977,141; 6,153,612; 6,297,247; 6,559,142; 6,555,535; 6,350,885; 5,627,206; 5,665,764; 5,958,972; 6,420,408; 6,492,422; 6,340,709; 6,022,948; 6,274,703; 6,294,694; 6,531,499; 6,465,508; 6,437,177; 6,376,665; 5,268,384; 5,183,900; 5,189,178; 6,511,993; 6,617,354; 6,331,563; 5,962,466; 5,861,427; 5,830,869; and 6,087,359.

23) NF Kappa B Inhibitors

In another embodiment, the pharmacologically active compound is a NF kappa B (NFKB) inhibitor (e.g., AVE-0545, Oxi-104 (benzamide, 4-amino-3-chloro-N-(2-(diethylamino)ethyl)-), dexlipotam, R-flurbiprofen ((1,1′-biphenyl)-4-acetic acid, 2-fluoro-alpha-methyl), SP100030 (2-chloro-N-(3,5-di(trifluoromethyl)phenyl)-4-(trifluoromethyl)pyrimidine-5-carboxamide), AVE-0545, Viatris, AVE-0547, Bay 11-7082, Bay 11-7085,15 deoxy-prostaylandin J2, bortezomib (boronic acid, ((1R)-3-methyl-1-(((2S)-1-oxo-3-phenyl-2-((pyrazinylcarbonyl)amino)propyl)amino)butyl)-, benzamide an d nicotinamide derivatives that inhibit NF-kappaB, such as those described in U.S. Pat. Nos. 5,561,161 and 5,340,565 (OxiGene), PG490-88Na, or an analogue or derivative thereof).

24) NO Agonists

In another embodiment, the pharmacologically active compound is a NO antagonist (e.g., NCX-4016 (benzoic acid, 2-(acetyloxy)-, 3-((nitrooxy)methyl)phenyl ester, NCX-2216, L-arginine or an analogue or derivative thereof).

25) P38 MAP Kinase Inhibitors

In another embodiment, the pharmacologically active compound is a p38 MAP kinase inhibitor (e.g., GW-2286, CGP-52411, BIRB-798, SB220025, RO-320-1195, RWJ-67657, RWJ-68354, SCIO-469, SCIO-323, AMG-548, CMC-146, SD-31145, CC-8866, Ro-320-1195, PD-98059 (4H-1-benzopyran-4-one, 2-(2-amino-3-methoxyphenyl)-), CGH-2466, doramapimod, SB-203580 (pyridine, 4-(5-(4-fluorophenyl)-2-(4-(methylsulfinyl)phenyl)-1H-imidazol-4-yl)-), SB-220025 ((5-(2-amino-4-pyrimidinyl)-4-(4-fluorophenyl)-1-(4-piperidinyl)imidazole), SB-281832, PD169316, SB202190, GSK-681323, EO-1606, GSK-681323, or an analogue or derivative thereof). Additional representative examples are included in U.S. Pat. Nos. 6,300,347; 6,316,464; 6,316,466; 6,376,527; 6,444,696; 6,479,507; 6,509,361; 6,579,874; 6,630,485, U.S. Patent Application Publication Nos. 2001/0044538A1; 2002/0013354A1; 2002/0049220A1; 2002/0103245A1; 2002/0151491A1; 2002/0156114A1; 2003/0018051A1; 2003/0073832A1; 2003/0130257A1; 2003/0130273A1; 2003/0130319A1; 2003/0139388A1; 20030139462A1; 2003/0149031A1; 2003/0166647A1; 2003/0181411A1; and PCT Publication Nos. WO 00/63204A2; WO 01/21591A1; WO 01/35959A1; WO 01/74811A2; WO 02/18379A2; WO 2064594A2; WO 2083622A2; WO 2094842A2; WO 2096426A1; WO 2101015A2; WO 2103000A2; WO 3008413A1; WO 3016248A2; WO 3020715A1; WO 3024899A2; WO 3031431A1; WO3040103A1; WO 3053940A1; WO 3053941A2; WO 3063799A2; WO 3079986A2; WO 3080024A2; WO 3082287A1; WO 97/44467A1; WO 99/01449A1; and WO 99/58523A1.

26) Phosphodiesterase Inhibitors

In another embodiment, the pharmacologically active compound is a phosphodiesterase inhibitor (e.g., CDP-840 (pyridine, 4-((2R)-2-(3-(cyclopentyloxy)-4-methoxyphenyl)-2-phenylethyl)-), CH-3697, CT-2820, D-22888 (imidazo(1,5-a)pyrido(3,2-e)pyrazin-6(5H)-one, 9-ethyl-2-methoxy-7-methyl-5-propyl-), D-4418 (8-methoxyquinoline-5-(N-(2,5-dichloropyridin-3-yl))carboxamide), 1-(3-cyclopentyloxy-4-methoxyphenyl)-2-(2,6-dichloro-4-pyridyl) ethanone oxime, D-4396, ONO-6126, CDC-998, CDC-801, V-11294A (3-(3-(cyclopentyloxy)-4-methoxybenzyl)-6-(ethylamino)-8-isopropyl-3H-purine hydrochloride), S,S′-methylene-bis(2-(8-cyclopropyl-3-propyl-6-(4-pyridylmethylamino)-2-thio-3H-purine)) tetrahyrochloride, rolipram (2-pyrrolidinone, 4-(3-(cyclopentyloxy)-4-methoxyphenyl)-), CP-293121, CP-353164 (5-(3-cyclopentyloxy-4-methoxyphenyl)pyridine-2-carboxamide), oxagrelate (6-phthalazinecarboxylic acid, 3,4-dihydro-1-(hydroxymethyl)-5,7-dimethyl-4-oxo-, ethyl ester), PD-168787, ibudilast (1-propanone, 2-methyl-1-(2-(1-methylethyl)pyrazolo(1,5-a)pyridin-3-yl)-), oxagrelate (6-phthalazinecarboxylic acid, 3,4-dihydro-1-(hydroxymethyl)-5,7-dimethyl-4-oxo-, ethyl ester), griseolic acid (alpha-L-talo-oct-4-enofuranuronic acid, 1-(6-amino-9H-purin-9-yl)-3,6-anhydro-6-C-carboxy-1,5-dideoxy-), KW-4490, KS-506, T-440, roflumilast (benzamide, 3-(cyclopropylmethoxy)-N-(3,5-dichloro-4-pyridinyl)-4-(difluoromethoxy)-), rolipram, milrinone, triflusinal (benzoic acid, 2-(acetyloxy)-4-(trifluoromethyl)-), anagrelide hydrochloride (imidazo(2,1-b)quinazolin-2(3H)-one, 6,7-dichloro-1,5-dihydro-, monohydrochloride), cilostazol (2(1H)-quinolinone, 6-(4-(1-cyclohexyl-1H-tetrazol-5-yl)butoxy)-3,4-dihydro-), propentofylline (1H-purine-2,6-dione, 3,7-dihydro-3-methyl-1-(5-oxohexyl)-7-propyl-), sildenafil citrate (piperazine, 1-((3-(4,7-dihydro-1-methyl-7-oxo-3-propyl-1H-pyrazolo(4,3-d)pyrimidin-5-yl)-4-ethoxyphenyl)sulfonyl)-4-methyl, 2-hydroxy-1,2,3-propanetricarboxylate-(1:1)), tadalafil (pyrazino(1′,2′:1,6)pyrido(3,4-b)indole1,4-dione, 6-(1,3-benzodioxol-5-yl)-2,3,6,7,12,12a-hexahydro-2-methyl-, (6R-trans)), vardenafil (piperazine, 1-(3-(1,4-dihydro-5-methyl(−4-oxo-7-propylimidazo(5,1-f)(1,2,4)-triazin-2-yl)-4-ethoxyphenyl)sulfonyl)-4-ethyl-), milrinone ((3,4′-bipyridine)-5-carbonitrile, 1,6-dihydro-2-methyl-6-oxo-), enoximone (2H-imidazol-2-one, 1,3-dihydro-4-methyl-5-(4-(methylthio)benzoyl)-), theophylline (1H-purine-2,6-dione, 3,7-dihydro-1,3-dimethyl-), ibudilast (1-propanone, 2-methyl-1-(2-(1-methylethyl)pyrazolo(1,5-a)pyridin-3-yl)-), aminophylline (1H-purine-2,6-dione, 3,7-dihydro-1,3-dimethyl-, compound with 1,2-ethanediamine (2:1)-), acebrophylline (7H-purine-7-acetic acid, 1,2,3,6-tetrahydro-1,3-dimethyl-2,6-dioxo-, compd. with trans-4-(((2-amino-3,5-dibromophenyl)methyl)amino)cyclohexanol (1:1)), plafibride (propanamide, 2-(4-chlorophenoxy)-2-methyl-N-(((4-morpholinylmethyl)amino)carbonyl)-), ioprinone hydrochloride (3-pyridinecarbonitrile, 1,2-dihydro-5-imidazo(1,2-a)pyridin-6-yl-6-methyl-2-oxo-, monohydrochloride-), fosfosal (benzoic acid, 2-(phosphonooxy)-), amrinone ((3,4′-bipyridin)-6(1H)-one, 5-amino-, or an analogue or derivative thereof).

Other examples of phosphodiesterase inhibitors include denbufylline (1H-purine-2,6-dione, 1,3-dibutyl-3,7-dihydro-7-(2-oxopropyl)-), propentofylline (1H-purine-2,6-dione, 3,7-dihydro-3-methyl-1-(5-oxohexyl)-7-propyl-) and pelrinone (5-pyrimidinecarbonitrile, 1,4-dihydro-2-methyl-4-oxo-6-[(3-pyridinylmethyl)amino]-).

Other examples of phosphodiesterase III inhibitors include enoximone (2H-imidazol-2-one, 1,3-dihydro-4-methyl-5-[4-(methylthio)benzoyl]-), and saterinone (3-pyridinecarbonitrile, 1,2-dihydro-5-[4-[2-hydroxy-3-[4-(2-methoxyphenyl)-1-piperazinyl]propoxy]phenyl]-6-methyl-2-oxo-).

Other examples of phosphodiesterase IV inhibitors include AWD-12-281, 3-auinolinecarboxylic acid, 1-ethyl-6-fluoro-1,4-dihydro-7-(4-methyl-1-piperazinyl)-4-oxo-), tadalafil (pyrazino(1′,2′:1,6)pyrido(3,4-b)indole1,4-dione, 6-(1,3-benzodioxol-5-yl)-2,3,6,7,12,12a-hexahydro-2-methyl-, (6R-trans)), and filaminast (ethanone, 1-[3-(cyclopentyloxy)-4-methoxyphenyl]-, O-(aminocarbonyl)oxime, (1E)-)

Another example of a phosphodiesterase V inhibitor is vardenafil (piperazine, 1-(3-(1,4-dihydro-5-methyl(−4-oxo-7-propylimidazo(5,1-f)(1,2,4)-triazin-2-yl)-4-ethoxyphenyl)sulfonyl)-4-ethyl-).

27) TGF Beta Inhibitors

In another embodiment, the pharmacologically active compound is a TGF beta Inhibitor (e.g., mannose-6-phosphate, LF-984, tamoxifen (ethanamine, 2-(4-(1,2-diphenyl-1-butenyl)phenoxy)-N,N-dimethyl-, (Z)-), tranilast, or an analogue or derivative thereof).

28) Thromboxane A2 Antagonists

In another embodiment, the pharmacologically active compound is a thromboxane A2 antagonist (e.g., CGS-22652 (3-pyridineheptanoic acid, ?-(4-(((4-chlorophenyl)sulfonyl)amino)butyl)-, (.+−.)-), ozagrel (2-propenoic acid, 3-(4-(1H-imidazol-1-ylmethyl)phenyl)-, (E)-), argatroban (2-piperidinecarboxylic acid, 1-(5-((aminoiminomethyl)amino)-1-oxo-2-(((1,2,3,4-tetrahydro-3-methyl-8-quinolinyl)sulfonyl)amino)pentyl)-4-methyl-), ramatroban (9H-carbazole-9-propanoic acid, 3-(((4-fluorophenyl)sulfonyl)amino)-1,2,3,4-tetrahydro-, (R)-), torasemide (3-pyridinesulfonamide, N-(((1-methylethyl)amino)carbonyl)-4-((3-methylphenyl)amino)-), gamma linoleic acid ((Z,Z,Z)-6,9,12-octadecatrienoic acid), seratrodast (benzeneheptanoic acid, zeta-(2,4,5-trimethyl-3,6-dioxo-1,4-cyclohexadien-1-yl)-, (+/−)-, or an analogue or derivative thereof).

29) TNFa Antagonists and TACE Inhibitors

In another embodiment, the pharmacologically active compound is a TNFa antagonist or TACE inhibitor (e.g., E-5531 (2-deoxy-6-O-(2-deoxy-3-O-(3(R)-(5(Z)-dodecenoyloxy)-decyl)-6-O-methyl-2-(3-oxotetradecanamido)-4-O-phosphono-β-D-glucopyranosyl)-3-0-(3(R)-hydroxydecyl)-2-(3-oxotetradecanamido)-alpha-D-glucopyranose-1-O-phosphate), AZD-4717, glycophosphopeptical, UR-12715 (B=benzoic acid, 2-hydroxy-5-((4-(3-(4-(2-methyl-1H-imidazol(4,5-c)pyridin-1-yl)methyl)-1-piperidinyl)-3-oxo-1-phenyl-1-propenyl)phenyl)azo) (Z)), PMS-601, AM-87, xyloadenosine (9H-purin-6-amine, 9-β-D-xylofuranosyl-), RDP-58, RDP-59, BB2275, benzydamine, E-3330 (undecanoic acid, 2-((4,5-dimethoxy-2-methyl-3,6-dioxo-1,4-cyclohexadien-1-yl)methylene)-, (E)-), N-(D,L-2-(hydroxyaminocarbonyl)methyl-4-methylpentanoyl)-L-3-(2′-naphthyl)alanyl-L-alanine, 2-aminoethyl amide, CP-564959, MLN-608, SPC-839, ENMD-0997, Sch-23863 ((2-(10,11-dihydro-5-ethoxy-5H-dibenzo (a,d) cyclohepten-S-yl)-N,N-dimethyl-ethanamine), SH-636, PKF-241-466, PKF-242-484, TNF-484A, cilomilast (cis-4-cyano-4-(3-(cyclopentyloxy)-4-methoxyphenyl)cyclohexane-1-carboxylic acid), GW-3333, GW-4459, BMS-561392, AM-87, cloricromene (acetic acid, ((8-chloro-3-(2-(diethylamino)ethyl)-4-methyl-2-oxo-2H-1-benzopyran-7-yl)oxy)-, ethyl ester), thalidomide (1H-Isoindole-1,3(2H)-dione, 2-(2,6-dioxo-3-piperidinyl)-), vesnarinone (piperazine, 1-(3,4-dimethoxybenzoyl)-4-(1,2,3,4-tetrahydro-2-oxo-6-quinolinyl)-), infliximab, lentinan, etanercept (1-235-tumor necrosis factor receptor (human) fusion protein with 236-467-immunoglobulin G1 (human gamma1-chain Fc fragment)), diacerein (2-anthracenecarboxylic acid, 4,5-bis(acetyloxy)-9,10-dihydro-9,10-dioxo-, or an analogue or derivative thereof).

30) Tyrosine Kinase Inhibitors

In another embodiment, the pharmacologically active compound is a tyrosine kinase inhibitor (e.g., SKI-606, ER-068224, SD-208, N-(6-benzothiazolyl)-4-(2-(1-piperazinyl)pyrid-5-yl)-2-pyrimidineamine, celastrol (24,25,26-trinoroleana-1(10),3,5,7-tetraen-29-oic acid, 3-hydroxy-9,13-dimethyl-2-oxo-, (9 beta., 13alpha,14β,20 alpha)-), CP-127374 (geldanamycin, 17-demethoxy-17-(2-propenylamino)-), CP-564959, PD-171026, CGP-52411 (1H-Isoindole-1,3(2H)-dione, 4,5-bis(phenylamino)-), CGP-53716 (benzamide, N-(4-methyl-3-((4-(3-pyridinyl)-2-pyrimidinyl)amino)phenyl)-), imatinib (4-((methyl-1-piperazinyl)methyl)-N-(4-methyl-3-((4-(3-pyridinyl)-2-pyrimidinyl)amino)-phenyl)benzamide methanesulfonate), NVP-MK980-NX, KF-250706 (13-chloro,5(R),6(S)-epoxy-14,16-dihydroxy-11-(hydroyimino)-3(R)-methyl-3,4,5,6, 11,12-hexahydro-1H-2-benzoxacyclotetradecin-1-one), 5-(3-(3-methoxy-4-(2-((E)-2-phenylethenyl)-4-oxazolylmethoxy)phenyl)propyl)-3-(2-((E)-2-phenylethenyl)-4-oxazolylmethyl)-2,4-oxazolidinedione, genistein, NV-06, or an analogue or derivative thereof).

31) Vitronectin Inhibitors

In another embodiment, the pharmacologically active compound is a vitronectin inhibitor (e.g., O-(9,10-dimethoxy-1,2,3,4,5,6-hexahydro-4-((1,4,5,6-tetrahydro-2-pyrimidinyl)hydrazono)-8-benz(e)azulenyl)-N-((phenylmethoxy)carbonyl)-DL-homoserine 2,3-dihydroxypropyl ester, (2S)-benzoylcarbonylamino-3-(2-((4S)-(3-(4,5-dihydro-1H-imidazol-2-ylamino)-propyl)-2,5-dioxo-imidazolidin-1-yl)-acetylamino)-propionate, Sch-221153, S-836, SC-68448 (β-((2-2-(((3-((aminoiminomethyl)amino)-phenyl)carbonyl)amino)acetyl)amino)-3,5-dichlorobenzenepropanoic acid), SD-7784, S-247, or an analogue or derivative thereof).

32) Fibroblast Growth Factor Inhibitors

In another embodiment, the pharmacologically active compound is a fibroblast growth factor inhibitor (e.g., CT-052923 (((2H-benzo(d)1,3-dioxalan-5-methyl)amino)(4-(6,7-dimethoxyquinazolin-4-yl)piperazinyl)methane-1-thione), or an analogue or derivative thereof).

33) Protein Kinase Inhibitors

In another embodiment, the pharmacologically active compound is a protein kinase inhibitor (e.g., KP-0201448, NPC15437 (hexanamide, 2,6-diamino-N-((1-(1-oxotridecyl)-2-piperidinyl)methyl)-), fasudil (1H-1,4-diazepine, hexahydro-1-(5-isoquinolinylsulfonyl)-), midostaurin (benzamide, N-(2,3,10,11,12,13-hexahydro-10-methoxy-9-methyl-1-oxo-9,13-epoxy-1H,9H-diindolo(1,2,3-gh:3′,2′,1′-Im)pyrrolo(3,4-j)(1,7)benzodiazonin-11-yl)-N-methyl-, (9Alpha,10β,11β,13Alpha)-), fasudil (1H-1,4-diazepine, hexahydro-1-(5-isoquinolinylsulfonyl)-, dexniguldipine (3,5-pyridinedicarboxylic acid, 1,4-dihydro-2,6-dimethyl-4-(3-nitrophenyl)-, 3-(4,4-diphenyl-1-piperidinyl)propyl methyl ester, monohydrochloride, (R)-), LY-317615 (1H-pyrole-2,5-dione, 3-(1-methyl-1H-indol-3-yl)-4-[1-[1-(2-pyridinylmethyl)-4-piperidinyl]-1H-indol-3-yl]-, monohydrochloride), perifosine (piperidinium, 4-[[hydroxy(octadecyloxy)phosphinyl]oxy]-1,1-dimethyl-, inner salt), LY-333531 (9H,18H-5,21:12,17-dimethenodibenzo(e,k)pyrrolo(3,4-h)(1,4,13)oxadiazacyclohexadecine-18,20(19H)-dione,9-((dimethylamino)methyl)-6,7,10,11-tetrahydro-, (S)-), Kynac; SPC-100270 (1,3-octadecanediol, 2-amino-, [S-(R*,R*)]-), Kynacyte, or an analogue or derivative thereof).

34) PDGF Receptor Kinase Inhibitors

In another embodiment, the pharmacologically active compound is a PDGF receptor kinase inhibitor (e.g., RPR-127963E, or an analogue or derivative thereof).

35) Endothelial Growth Factor Receptor Kinase Inhibitors

In another embodiment, the pharmacologically active compound is an endothelial growth factor receptor kinase inhibitor (e.g., CEP-7055, SU-0879 ((E)-3-(3,5-di-tert-butyl-4-hydroxyphenyl)-2-(aminothiocarbonyl)acrylonitrile), BIBF-1000, AG-013736 (CP-868596), AMG-706, AVE-0005, NM-3 (3-(2-methylcarboxymethyl)-6-methoxy-8-hydroxy-isocoumarin), Bay-43-9006, SU-011248, or an analogue or derivative thereof).

36) Retinoic Acid Receptor Antagonists

In another embodiment, the pharmacologically active compound is a retinoic acid receptor antagonist (e.g., etarotene (Ro-15-1570) (naphthalene, 6-(2-(4-(ethylsulfonyl)phenyl)-1-methylethenyl)-1,2,3,4-tetrahydro-1,1,4,4-tetramethyl-, (E)-), (2E,4E)-3-methyl-5-(2-((E)-2-(2,6,6-trimethyl-1-cyclohexen-1-yl)ethenyl)-1-cyclohexen-1-yl)-2,4-pentadienoic acid, tocoretinate (retinoic acid, 3,4-dihydro-2,5,7,8-tetramethyl-2-(4,8,12-trimethyltridecyl)-2H-1-benzopyran-6-yl ester, (2R*(4R*,8R*))-(O)-), aliretinoin (retinoic acid, cis-9, trans-13-), bexarotene (benzoic acid, 4-(1-(5,6,7,8-tetrahydro-3,5,5,8,8-pentamethyl-2-naphthalenyl)ethenyl)-), tocoretinate (retinoic acid, 3,4-dihydro-2,5,7,8-tetramethyl-2-(4,8,12-trimethyltridecyl)-2H-1-benzopyran-6-yl ester, [2R*(4R*,8R*)]-(O)-, or an analogue or derivative thereof).

37) Platelet Derived Growth Factor Receptor Kinase Inhibitors

In another embodiment, the pharmacologically active compound is a platelet derived growth factor receptor kinase inhibitor (e.g., leflunomide (4-isoxazolecarboxamide, 5-methyl-N-(4-(trifluoromethyl)phenyl)-, or an analogue or derivative thereof).

38) Fibronogin Antagonists

In another embodiment, the pharmacologically active compound is a fibrinogin antagonist (e.g., picotamide (1,3-benzenedicarboxamide, 4-methoxy-N,N′-bis(3-pyridinylmethyl)-, or an analogue or derivative thereof).

39) Antimycotic Agents

In another embodiment, the pharmacologically active compound is an antimycotic agent (e.g., miconazole, sulconizole, parthenolide, rosconitine, nystatin, isoconazole, fluconazole, ketoconasole, imidazole, itraconazole, terpinafine, elonazole, bifonazole, clotrimazole, conazole, terconazole (piperazine, 1-(4-((2-(2,4-dichlorophenyl)-2-(1H-1,2,4-triazol-1-ylmethyl)-1,3-dioxolan-4-yl)methoxy)phenyl)-4-(1-methylethyl)-, cis-), isoconazole (1-(2-(2-6-dichlorobenzyloxy)-2-(2-,4-dichlorophenyl)ethyl)), griseofulvin (spiro(benzofuran-2(3H),1′-(2)cyclohexane)-3,4′-dione, 7-chloro-2′,4,6-trimeth-oxy-6′methyl-, (1′S-trans)-), bifonazole (1H-imidazole, 1-((1,1′-biphenyl)-4-ylphenylmethyl)-), econazole nitrate (1-(2-((4-chlorophenyl)methoxy)-2-(2,4-dichlorophenyl)ethyl)-1H-imidazole nitrate), croconazole (1H-imidazole, 1-(1-(2-((3-chlorophenyl)methoxy)phenyl)ethenyl)-), sertaconazole (1H-Imidazole, 1-(2-((7-chlorobenzo(b)thien-3-yl)methoxy)-2-(2,4-dichlorophenyl)ethyl)-), omoconazole (1H-imidazole, 1-(2-(2-(4-chlorophenoxy)ethoxy)-2-(2,4-dichlorophenyl)-1-methylethenyl)-, (Z)-), flutrimazole (1H-imidazole, 1-((2-fluorophenyl)(4-fluorophenyl)phenylmethyl)-), fluconazole (1H-1,2,4-triazole-1-ethanol, alpha-(2,4-difluorophenyl)-alpha-(1H-1,2,4-triazol-1-ylmethyl)-), neticonazole (1H-Imidazole, 1-(2-(methylthio)-1-(2-(pentyloxy)phenyl)ethenyl)-, monohydrochloride, (E)-), butoconazole (1H-imidazole, 1-(4-(4-chlorophenyl)-2-((2,6-dichlorophenyl)thio)butyl)-, (+/−)-), clotrimazole (1-((2-chlorophenyl)diphenylmethyl)-1H-imidazole, or an analogue or derivative thereof).

40) Bisphosphonates

In another embodiment, the pharmacologically active compound is a bisphosphonate (e.g., clodronate, alendronate, pamidronate, zoledronate, or an analogue or derivative thereof).

41) Phospholipase A1 Inhibitors

In another embodiment, the pharmacologically active compound is a phospholipase A1 inhibitor (e.g., ioteprednol etabonate (androsta-1,4-diene-17-carboxylic acid, 17-((ethoxycarbonyl)oxy)-11-hydroxy-3-oxo-, chloromethyl ester, (11β,17 alpha)-, or an analogue or derivative thereof).

42) Histamine H1/H2/H3 Receptor Antagonists

In another embodiment, the pharmacologically active compound is a histamine H1, H2, or H3 receptor antagonist (e.g., ranitidine (1,1-ethenediamine, N-(2-(((5-((dimethylamino)methyl)-2-furanyl)methyl)thio)ethyl)-N′-methyl-2-nitro-), niperotidine (N-(2-((5-((dimethylamino)methyl)furfuryl)thio)ethyl)-2-nitro-N′-piperonyl-1,1-ethenediamine), famotidine (propanimidamide, 3-(((2-((aminoiminomethyl)amino)-4-thiazolyl)methyl)thio)-N-(aminosulfonyl)-), roxitadine acetate HCl (acetamide, 2-(acetyloxy)-N-(3-(3-(1-piperidinylmethyl)phenoxy)propyl)-, monohydrochloride), lafutidine (acetamide, 2-((2-furanylmethyl)sulfinyl)-N-(4-((4-(1-piperidinylmethyl)-2-pyridinyl)oxy)-2-butenyl)-, (Z)-), nizatadine (1,1-ethenediamine, N-(2-(((2-((dimethylamino)methyl)-4-thiazolyl)methyl)thio)ethyl)-N′-methyl-2-nitro-), ebrotidine (benzenesulfonamide, N-(((2-(((2-((aminoiminomethyl)amino)-4-thiazoly)methyl)thio)ethyl)amino)methylene)-4-bromo-), rupatadine (5H-benzo(5,6)cyclohepta(1,2-b)pyridine, 8-chloro-6,11-dihydro-11-(1-((5-methyl-3-pyridinyl)methyl)-4-piperidinylidene)-, trihydrochloride-), fexofenadine HCl (benzeneacetic acid, 4-(1-hydroxy-4-(4(hydroxydiphenylmethyl)-1-piperidinyl)butyl)-alpha, alpha-dimethyl-, hydrochloride, or an analogue or derivative thereof).

43) Macrolide Antibiotics

In another embodiment, the pharmacologically active compound is a macrolide antibiotic (e.g., dirithromycin (erythromycin, 9-deoxo-11-deoxy-9,11-(imino(2-(2-methoxyethoxy)ethylidene)oxy)-, (9S(R))-), flurithromycin ethylsuccinate (erythromycin, 8-fluoro-mono(ethyl butanedioate) (ester)-), erythromycin stinoprate (erythromycin, 2′-propanoate, compound with N-acetyl-L-cysteine (1:1)), clarithromycin (erythromycin, 6-O-methyl-), azithromycin (9-deoxo-9a-aza-9a-methyl-9a-homoerythromycin-A), telithromycin (3-de((2,6-dideoxy-3-C-methyl-3-O-methyl-alpha-L-ribo-hexopyranosyl)oxy)-11,12-dideoxy-6-O-methyl-3-oxo-12,11-(oxycarbonyl((4-(4-(3-pyridinyl)-1H-imidazol-1-yl)butyl)imino))-), roxithromycin (erythromycin, 9-(O-((2-methoxyethoxy)methyl)oxime)), rokitamycin (leucomycin V, 4B-butanoate 3B-propanoate), RV-11 (erythromycin monopropionate mercaptosuccinate), midecamycin acetate (leucomycin V, 3B,9-diacetate 3,4B-dipropanoate), midecamycin (leucomycin V, 3,4B-dipropanoate), josamycin (leucomycin V, 3-acetate 4B-(3-methylbutanoate), or an analogue or derivative thereof).

44) GPIIb IIIa Receptor Antagonists

In another embodiment, the pharmacologically active compound is a GPIIb IIIa receptor antagonist (e.g., tirofiban hydrochloride (L-tyrosine, N-(butylsulfonyl)-O-(4-(4-piperid inyl)butyl)-, monohydrochloride-), eptifibatide (L-cysteinamide, N6-(aminoiminomethyl)-N-2-(3-mercapto-1-oxopropyl)-L-lysylglycyl-L-alpha-aspartyl-L-tryptophyl-L-prolyl-, cyclic(1->6)-disulfide), xemilofiban hydrochloride, or an analogue or derivative thereof).

45) Endothelin Receptor Antagonists

In another embodiment, the pharmacologically active compound is an endothelin receptor antagonist (e.g., bosentan (benzenesulfonamide, 4-(1,1-dimethylethyl)-N-(6-(2-hydroxyethoxy)-5-(2-methoxyphenoxy)(2,2′-bipyrimidin)-4-yl)-, or an analogue or derivative thereof).

46) Peroxisome Proliferator-Activated Receptor Agonists

In another embodiment, the pharmacologically active compound is a peroxisome proliferator-activated receptor agonist (e.g., gemfibrozil (pentanoic acid, 5-(2,5-dimethylphenoxy)-2,2-dimethyl-), fenofibrate (propanoic acid, 2-(4-(4-chlorobenzoyl)phenoxy)-2-methyl-, 1-methylethyl ester), ciprofibrate (propanoic acid, 2-(4-(2,2-dichlorocyclopropyl)phenoxy)-2-methyl-), rosiglitazone maleate (2,4-thiazolidinedione, 5-((4-(2-(methyl-2-pyridinylamino)ethoxy)phenyl)methyl)-, (Z)-2-butenedioate (1:1)), pioglitazone hydrochloride (2,4-thiazolidinedione, 5-((4-(2-(5-ethyl-2-pyridinyl)ethoxy)phenyl)methyl)-, monohydrochloride (+/−)-), etofylline clofibrate (propanoic acid, 2-(4-chlorophenoxy)-2-methyl-, 2-(1,2,3,6-tetrahydro-1,3-dimethyl-2,6-dioxo-7H-purin-7-yl)ethyl ester), etofibrate (3-pyridinecarboxylic acid, 2-(2-(4-chlorophenoxy)-2-methyl-1-oxopropoxy)ethyl ester), clinofibrate (butanoic acid, 2,2′-(cyclohexylidenebis(4,1-phenyleneoxy))bis(2-methyl-)), bezafibrate (propanoic acid, 2-(4-(2-((4-chlorobenzoyl)amino)ethyl)phenoxy)-2-methyl-), binifibrate (3-pyridinecarboxylic acid, 2-(2-(4-chlorophenoxy)-2-methyl-1-oxopropoxy)-1,3-propanediyl ester), or an analogue or derivative thereof).

In one aspect, the pharmacologically active compound is a peroxisome proliferator-activated receptor alpha agonist, such as GW-590735, GSK-677954, GSK501516, pioglitazone hydrochloride (2,4-thiazolidinedione, 5-[[4-[2-(5-ethyl-2-pyridinyl)ethoxy]phenyl]methyl]-, monohydrochloride (+/−)-, or an analogue or derivative thereof).

47) Estrogen Receptor Agents

In another embodiment, the pharmacologically active compound is an estrogen receptor agent (e.g., estradiol, 17-β-estradiol, or an analogue or derivative thereof).

48) Somatostatin Analogues

In another embodiment, the pharmacologically active compound is a somatostatin analogue (e.g., angiopeptin, or an analogue or derivative thereof).

49) Neurokinin 1 Antagonists

In another embodiment, the pharmacologically active compound is a neurokinin 1 antagonist (e.g., GW-597599, lanepitant ((1,4′-bipiperidine)-1′-acetamide, N-(2-(acetyl((2-methoxyphenyl)methyl)amino)-1-(1H-indol-3-ylmethyl)ethyl)-(R)-), nolpitantium chloride (1-azoniabicyclo[2.2.2]octane, 1-[2-[3-(3,4-dichlorophenyl)-1-[[3-(1-methylethoxy)phenyl]acetyl]-3-piperidinyl]ethyl]-4-phenyl-, chloride, (S)-), or saredutant (benzamide, N-[4-[4-(acetylamino)-4-phenyl-1-piperidinyl]-2-(3,4-dichlorophenyl)butyl]-N-methyl-, (S)-), or vofopitant (3-piperid inamine, N-[[2-methoxy-5-[5-(trifluoromethyl)-1H-tetrazol-1-yl]phenyl]methyl]-2-phenyl-, (2S,3S)-, or an analogue or derivative thereof).

50) Neurokinin 3 Antagonist

In another embodiment, the pharmacologically active compound is a neurokinin 3 antagonist (e.g., talnetant (4-quinolinecarboxamide, 3-hydroxy-2-phenyl-N-[(1S)-1-phenylpropyl]-, or an analogue or derivative thereof.

51) Neurokinin Antagonist

In another embodiment, the pharmacologically active compound is a neurokinin antagonist (e.g., GSK-679769, GSK-823296, SR-489686 (benzamide, N-[4-[4-(acetylamino)-4-phenyl-1-piperidinyl]-2-(3,4-dichlorophenyl)butyl]-N-methyl-, (S)-), SB-223412; SB-235375 (4-quinolinecarboxamide, 3-hydroxy-2-phenyl-N-[(1S)-1-phenylpropyl]-), UK-226471, or an analogue or derivative thereof).

52) VLA-4 Antagonist

In another embodiment, the pharmacologically active compound is a VLA-4 antagonist (e.g., GSK683699, or an analogue or derivative thereof).

53) Osteoclast Inhibitor

In another embodiment, the pharmacologically active compound is a osteoclast inhibitor (e.g., ibandronic acid (phosphonic acid, [1-hydroxy-3-(methylpentylamino)propylidene]bis-), alendronate sodium, or an analogue or derivative thereof).

54) DNA topoisomerase ATP Hydrolysing Inhibitor

In another embodiment, the pharmacologically active compound is a DNA topoisomerase ATP hydrolysing inhibitor (e.g., enoxacin (1,8-naphthyridine-3-carboxylic acid, 1-ethyl-6-fluoro-1,4-dihydro-4-oxo-7-(1-piperazinyl)-), levofloxacin (7H-Pyrido[1,2,3-de]-1,4-benzoxazine-6-carboxylic acid, 9-fluoro-2,3-dihydro-3-methyl-10-(4-methyl-1-piperazinyl)-7-oxo-, (S)-), ofloxacin (7H-pyrido[1,2,3-de]-1,4-benzoxazine-6-carboxylic acid, 9-fluoro-2,3-dihydro-3-methyl-10-(4-methyl-1-piperazinyl)-7-oxo-, (+/−)-), pefloxacin (3-quinolinecarboxylic acid, 1-ethyl-6-fluoro-1,4-dihydro-7-(4-methyl-1-piperazinyl)-4-oxo-), pipemidic acid (pyrido[2,3-d]pyrimidine-6-carboxylic acid, 8-ethyl-5,8-dihydro-5-oxo-2-(1-piperazinyl)-), pirarubicin (5,12-naphthacenedione, 10-[[-amino-2,3,6-trideoxy-4-O-(tetrahydro-2H-pyran-2-yl)-alpha-L-lyxo-hexopyranosyl]oxy]-7,8,9,10-tetrahydro-6,8,11-trihydroxy-8-(hydroxyacetyl)-1-methoxy-, [8S-[8 alpha,10 alpha(S*)]]-), sparfloxacin (3-quinolinecarboxylic acid, 5-amino-1-cyclopropyl-7-(3,5-dimethyl-1-piperazinyl)-6,8-difluoro-1,4-dihydro-4-oxo-, cis-), AVE-6971, cinoxacin ([1,3]dioxolo[4,5-g]cinnoline-3-carboxylic acid, 1-ethyl-1,4-dihydro-4-oxo-), or an analogue or derivative thereof).

55) Angiotensin I Converting Enzyme Inhibitor

In another embodiment, the pharmacologically active compound is an angiotensin I converting enzyme inhibitor (e.g., ramipril (cyclopenta[b]pyrrole-2-carboxylic acid, 1-[2-[[1-(ethoxycarbonyl)-3-phenylpropyl]amino]-1-oxopropyljoctahydro-, [2S-[1[R*(R*)],2 alpha,3β,6aβ]]-), trandolapril (1H-indole-2-carboxylic acid, 1-[2-[(1-carboxy-3-phenylpropyl)amino]-1-oxopropyl]octahydro-, [2S-[1[R*(R*)],2 alpha,3a alpha,7aβ]]-), fasidotril (L-alanine, N-[(2S)-3-(acetylthio)-2-(1,3-benzodioxol-5-ylmethyl)-1-oxopropyl]-, phenylmethyl ester), cilazapril (6H-pyridazino[1,2-a][1,2]diazepine-1-carboxylic acid, 9-[[1-(ethoxycarbonyl)-3-phenylpropyl]amino]octahydro-10-oxo-, [1S-[1 alpha, 9 alpha(R*)]]-), ramipril (cyclopenta[b]pyrrole-2-carboxylic acid, 1-[2-[[1-(ethoxycarbonyl)-3-phenylpropyl]amino]-1-oxopropyl]octahydro-, [2S-[1[R*(R*)], 2 alpha,3β,6aβ]]-, or an analogue or derivative thereof).

56) Angiotensin II Antagonist

In another embodiment, the pharmacologically active compound is an angiotensin II antagonist (e.g., HR-720 (1H-imidazole-5-carboxylic acid, 2-butyl-4-(methylthio)-1-[[2′-[[[(propylamino)carbonyl]amino]sulfonyl][1,1′-biphenyl]-4-yl]methyl]-, dipotassium salt, or an analogue or derivative thereof).

57) Enkephalinase Inhibitor

In another embodiment, the pharmacologically active compound is an enkephalinase inhibitor (e.g., Aventis 100240 (pyrido[2,1-a][2]benzazepine-4-carboxylic acid, 7-[[2-(acetylthio)-1-oxo-3-phenylpropyl]amino]-1,2,3,4,6,7,8,12b-octahydro-6-oxo-, [4S-[4 alpha, 7 alpha(R*),12bβ]]-), AVE-7688, or an analogue or derivative thereof).

58) Peroxisome Proliferator-Activated Receptor Gamma Agonist Insulin Sensitizer

In another embodiment, the pharmacologically active compound is peroxisome proliferator-activated receptor gamma agonist insulin sensitizer (e.g., rosiglitazone maleate (2,4-thiazolidinedione, 5-((4-(2-(methyl-2-pyridinylamino)ethoxy)phenyl)methyl)-, (Z)-2-butenedioate (1:1), farglitazar (GI-262570, GW-2570, GW-3995, GW-5393, GW-9765), LY-929, LY-519818, LY-674, or LSN-862), or an analogue or derivative thereof).

59) Protein Kinase C Inhibitor

In another embodiment, the pharmacologically active compound is a protein kinase C inhibitor, such as ruboxistaurin mesylate (9H,18H-5,21:12,17-dimethenodibenzo(e,k)pyrrolo(3,4-h)(1,4,13)oxadiazacyclohexadecine-18,20(19H)-dione,9-((dimethylamino)methyl)-6,7,10,11-tetrahydro-, (S)-), safingol (1,3-octadecanediol, 2-amino-, [S-(R*,R*)]-), or enzastaurin hydrochloride (1H-pyrole-2,5-dione, 3-(1-methyl-1H-indol-3-yl)-4-[1-[1-(2-pyridinylmethyl)-4-piperidinyl]-1H-indol-3-yl]-, monohydrochloride), or an analogue or derivative thereof.

60) ROCK (rho-Associated Kinase) Inhibitors

In another embodiment, the pharmacologically active compound is a ROCK (rho-associated kinase) inhibitor, such as Y-27632, HA-1077, H-1152 and 4-1-(aminoalkyl)-N-(4-pyridyl) cyclohexanecarboxamide or an analogue or derivative thereof.

61) CXCR3 Inhibitors

In another embodiment, the pharmacologically active compound is a CXCR3 inhibitor such as T-487, T0906487 or analogue or derivative thereof.

62) Itk Inhibitors

In another embodiment, the pharmacologically active compound is an Itk inhibitor such as BMS-509744 or an analogue or derivative thereof.

63) Cytosolic phospholipase A₂-Alpha Inhibitors

In another embodiment, the pharmacologically active compound is a cytosolic phospholipase A₂-alpha inhibitor such as efipladib (PLA-902) or analogue or derivative thereof.

64) PPAR Agonist

In another embodiment, the pharmacologically active compound is a PPAR Agonist (e.g., Metabolex ((−)-benzeneacetic acid, 4-chloro-alpha-[3-(trifluoromethyl)-phenoxy]-, 2-(acetylamino)ethyl ester), balaglitazone (5-(4-(3-methyl-4-oxo-3,4-dihydro-quinazolin-2-yl-methoxy)-benzyl)-thiazolidine-2,4-dione), ciglitazone (2,4-thiazolidinedione, 5-[[4-[(1-methylcyclohexyl)methoxy]phenyl]methyl]-), DRF-10945, farglitazar, GSK-677954, GW-409544, GW-501516, GW-590735, GW-590735, K-111, KRP-101, LSN-862, LY-519818, LY-674, LY-929, muraglitazar; BMS-298585 (Glycine, N-[(4-methoxyphenoxy)carbonyl]-N-[[4-[2-(5-methyl-2-phenyl-4-oxazolyl)ethoxy]phenyl]methyl]-), netoglitazone; isaglitazone (2,4-thiazolidinedione, 5-[[6-[(2-fluorophenyl)methoxy]-2-naphthalenyl]methyl]-), Actos AD-4833; U-72107A (2,4-thiazolidinedione, 5-[[4-[2-(5-ethyl-2-pyridinyl)ethoxy]phenyl]methyl]-, monohydrochloride (+/−)-), JTT-501; PNU-182716 (3,5-Isoxazolidinedione, 4-[[4-[2-(5-methyl-2-phenyl-4-oxazolyl)ethoxy]phenyl]methyl]-), AVANDIA (from SB Pharmco Puerto Rico, Inc. (Puerto Rico); BRL-48482; BRL-49653; BRL-49653c; NYRACTA and Venvia (both from (SmithKline Beecham (United Kingdom)); tesaglitazar ((2S)-2-ethoxy-3-[4-[2-[4-[(methylsulfonyl)oxy]phenyl]ethoxy]phenyl]propanoic acid), troglitazone (2,4-Thiazolid inedione, 5-[[4-[(3,4-dihydro-6-hydroxy-2,5,7,8-tetramethyl-2H-1-benzopyran-2-yl)methoxy]phenyl]methyl]-), and analogues and derivatives thereof).

65) Immunosuppressants

In another embodiment, the pharmacologically active compound is an immunosuppressant (e.g., batebulast (cyclohexanecarboxylic acid, 4-[[(aminoiminomethyl)amino]methyl]-, 4-(1,1-dimethylethyl)phenyl ester, trans-), cyclomunine, exalamide (benzamide, 2-(hexyloxy)-), LYN-001, CCl-779 (rapamycin 42-(3-hydroxy-2-(hydroxymethyl)-2-methylpropanoate)), 1726; 1726-D; AVE-1726, or an analogue or derivative thereof).

66) Erb Inhibitor

In another embodiment, the pharmacologically active compound is an Erb inhibitor (e.g., canertinib dihydrochloride (N-[4-(3-(chloro-4-fluoro-phenylamino)-7-(3-morpholin-4-yl-propoxy)-quinazolin-6-yl]-acrylamide dihydrochloride), CP-724714, or an analogue or derivative thereof).

67) Apoptosis Agonist

In another embodiment, the pharmacologically active compound is an apoptosis agonist (e.g., CEFLATONIN (CGX-635) (from Chemgenex Therapeutics, Inc., Menlo Park, Calif.), CHML, LBH-589, metoclopramide (benzamide, 4-amino-5-chloro-N-[2-(diethylamino)ethyl]-2-methoxy-), patupilone (4,17-dioxabicyclo(14.1.0)heptadecane-5,9-dione, 7,11-dihydroxy-8,8,10,12,16-pentamethyl-3-(1-methyl-2-(2-methyl-4-thiazolyl)ethenyl, (1R,3S,7S,10R,11S,12S,16R)), AN-9; pivanex (butanoic acid, (2,2-dimethyl-1-oxopropoxy)methyl ester), SL-100; SL-102; SL-11093; SL-11098; SL-11099; SL-93; SL-98; SL-99, or an analogue or derivative thereof).

68) Lipocortin Agonist

In another embodiment, the pharmacologically active compound is an lipocortin agonist (e.g., CGP-13774 (9Alpha-chloro-6Alpha-fluoro-11β,17alpha-dihydroxy-16Alpha-methyl-3-oxo-1,4- and rostadiene-17-carboxylic acid-methylester-17-propionate), or analogue or derivative thereof).

69) VCAM-1 Antagonist

In another embodiment, the pharmacologically active compound is a VCAM-1 antagonist (e.g., DW-908e, or an analogue or derivative thereof).

70) Collagen Antagonist

In another embodiment, the pharmacologically active compound is a collagen antagonist (e.g., E-5050 (Benzenepropanamide, 4-(2,6-dimethylheptyl)-N-(2-hydroxyethyl)-β-methyl-), Iufironil (2,4-Pyridined icarboxamide, N,N′-bis(2-methoxyethyl)-), or an analogue or derivative thereof).

71) Alpha 2 Integrin Antagonist

In another embodiment, the pharmacologically active compound is an alpha 2 integrin antagonist (e.g., E-7820, or an analogue or derivative thereof).

72) TNF Alpha Inhibitor

In another embodiment, the pharmacologically active compound is a TNF alpha inhibitor (e.g., ethyl pyruvate, Genz-29155, lentinan (Ajinomoto Co., Inc. (Japan)), linomide (3-quinolinecarboxamide, 1,2-dihydro-4-hydroxy-N,1-dimethyl-2-oxo-N-phenyl-), UR-1505, or an analogue or derivative thereof).

73) Nitric Oxide Inhibitor

In another embodiment, the pharmacologically active compound is a nitric oxide inhibitor (e.g., guanidioethyldisulfide, or an analogue or derivative thereof).

74) Cathepsin Inhibitor

In another embodiment, the pharmacologically active compound is a cathepsin inhibitor (e.g., SB-462795 or an analogue or derivative therof).

Within various embodiments of the invention, a device incorporates or is coated on one aspect, portion or surface with a composition which inhibits fibrosis (and/or restenosis), as well as with a composition or compound which promotes fibrosis on another aspect, portion or surface of the device. Representative examples of agents that promote fibrosis include silk and other irritants (e.g., talc, wool (including animal wool, wood wool, and synthetic wool), talcum powder, copper, metallic beryllium (or its oxides), quartz dust, silica, crystalline silicates), polymers (e.g., polylysine, polyurethanes, poly(ethylene terephthalate), PTFE, poly(alkylcyanoacrylates), and poly(ethylene-co-vinylacetate); vinyl chloride and polymers of vinyl chloride; peptides with high lysine content; growth factors and inflammatory cytokines involved in angiogenesis, fibroblast migration, fibroblast proliferation, ECM synthesis and tissue remodeling, such as epidermal growth factor (EGF) family, transforming growth factor-α (TGF-α), transforming growth factor-β (TGF-9-1, TGF-9-2, TGF-9-3, platelet-derived growth factor (PDGF), fibroblast growth factor (acidic—aFGF; and basic—bFGF), fibroblast stimulating factor-1, activins, vascular endothelial growth factor (including VEGF-2, VEGF-3, VEGF-A, VEGF-B, VEGF-C, placental growth factor—PIGF), angiopoietins, insulin-like growth factors (IGF), hepatocyte growth factor (HGF), connective tissue growth factor (CTGF), myeloid colony-stimulating factors (CSFs), monocyte chemotactic protein, granulocyte-macrophage colony-stimulating factors (GM-CSF), granulocyte colony-stimulating factor (G-CSF), macrophage colony-stimulating factor (M-CSF), erythropoietin, interleukins (particularly IL-1, IL-8, and IL-6), tumor necrosis factor-α (TNF9), nerve growth factor (NGF), interferon-α, interferon-β, histamine, endothelin-1, angiotensin II, growth hormone (GH), and synthetic peptides, analogues or derivatives of these factors are also suitable for release from specific implants and devices to be described later. Other examples include CTGF (connective tissue growth factor); inflammatory microcrystals (e.g., crystalline minerals such as crystalline silicates); bromocriptine, methylsergide, methotrexate, chitosan, N-carboxybutyl chitosan, carbon tetrachloride, thioacetamide, fibrosin, ethanol, bleomycin, naturally occurring or synthetic peptides containing the Arg-Gly-Asp (RGD) sequence, generally at one or both termini (see, e.g., U.S. Pat. No. 5,997,895), and tissue adhesives, such as cyanoacrylate and crosslinked poly(ethylene glycol)-methylated collagen compositions. Other examples of fibrosis-inducing agents include bone morphogenic proteins (e.g., BMP-2, BMP-3, BMP-4, BMP-5, BMP-6 (Vgr-1), BMP-7 (OP-1), BMP-8, BMP-9, BMP-10, BMP-11, BMP-12, BMP-13, BMP-14, BMP-15, and BMP-16. Of these, BMP-2, BMP-3, BMP-4, BMP-5, BMP-6, and BMP-7 are of particular utility. Bone morphogenic proteins are described, for example, in U.S. Pat. Nos. 4,877,864; 5,013,649; 5,661,007; 5,688,678; 6,177,406; 6,432,919; and 6,534,268 and Wozney, J. M., et al. (1988) Science: 242(4885); 1528-1534.

Other representative examples of fibrosis-inducing agents include components of extracellular matrix (e.g., fibronectin, fibrin, fibrinogen, collagen (e.g., bovine collagen), including fibrillar and non-fibrillar collagen, adhesive glycoproteins, proteoglycans (e.g., heparin sulfate, chondroitin sulfate, dermatan sulfate), hyaluronan, secreted protein acidic and rich in cysteine (SPARC), thrombospondins, tenacin, and cell adhesion molecules (including integrins, vitronectin, fibronectin, laminin, hyaluronic acid, elastin, bitronectin), proteins found in basement membranes, and fibrosin) and inhibitors of matrix metalloproteinases, such as TIMPs (tissue inhibitors of matrix metalloproteinases) and synthetic TIMPs, such as, e.g., marimistat, batimistat, doxycycline, tetracycline, minocycline, TROCADE, Ro-1130830, CGS 27023A, and BMS-275291 and analogues and derivatives thereof.

The medical implant may include a fibrosis-inhibiting agent and an anti-thrombotic agent and/or antiplatelet agent and/or a thrombolytic agent, which reduces the likelihood of thrombotic events upon implantation of a medical implant. Within various embodiments of the invention, a device is coated on one aspect with a composition which inhibits fibrosis (and/or restenosis), as well as being coated with a composition or compound which prevents thrombosis on another aspect of the device. Representative examples of anti-thrombotic and/or antiplatelet and/or thrombolytic agents include heparin, heparin fragments, organic salts of heparin, heparin complexes (e.g., benzalkonium heparinate, tridodecylammonium heparinate), dextran, sulfonated carbohydrates such as dextran sulphate, coumadin, coumarin, heparinoid, danaparoid, argatroban chitosan sulfate, chondroitin sulfate, danaparoid, lepirudin, hirudin, AMP, adenosine, 2-chloroadenosine, acetylsalicylic acid, phenylbutazone, indomethacin, meclofenamate, hydrochloroquine, dipyridamole, iloprost, streptokinase, factor Xa inhibitors, such as DX9065a, magnesium, and tissue plasminogen activator. Further examples include plasminogen, lys-plasminogen, alpha-2-antiplasmin, urokinase, aminocaproic acid, ticlopidine, clopidogrel, trapidil (triazolopyrimidine), naftidrofuryl, auriritricarboxylic acid and glycoprotein llb/Illa inhibitors such as abcixamab, eptifibatide, and tirogiban. Other agents capable of affecting the rate of clotting include glycosaminoglycans, danaparoid, 4-hydroxycourmarin, warfarin sodium, dicumarol, phenprocoumon, indan-1,3-dione, acenocoumarol, anisindione, and rodenticides including bromadiolone, brodifacoum, diphenadione, chlorophacinone, and pidnone.

The thrombogenicity of a medical implant may be reduced by coating the implant with a polymeric formulation that has anti-thrombogenic properties. For example, a medical device may be coated with a hydrophilic polymer gel. The polymer gel can comprise a hydrophilic, biodegradable polymer that is physically removed from the surface of the device over time, thus reducing adhesion of platelets to the device surface. The gel composition can include a polymer or a blend of polymers. Representative examples include alginates, chitosan and chitosan sulfate, hyaluronic acid, dextran sulfate, PLURONIC polymers (e.g., F-127 or F87), chain extended PLURONIC polymers, various polyester-polyether block copolymers of various configurations (e.g., AB, ABA, or BAB, where A is a polyester such as PLA, PGA, PLGA, PCL or the like), examples of which include MePEG-PLA, PLA-PEG-PLA, and the like). In one embodiment, the anti-thrombotic composition can include a crosslinked gel formed from a combination of molecules (e.g., PEG) having two or more terminal electrophilic groups and two or more nucleophilic groups.

In one aspect, the present invention also provides for the combination of a medical implant (as well as compositions and methods for making medical implants) that includes an anti-fibrosing agent and an anti-infective agent, which reduces the likelihood of infections in medical implants. Infection is a common complication of the implantation of foreign bodies such as medical devices. Foreign materials provide an ideal site for micro-organisms to attach and colonize. It is also hypothesized that there is an impairment of host defenses to infection in the microenvironment surrounding a foreign material. These factors make medical implants particularly susceptible to infection and make eradication of such an infection difficult, if not impossible, in most cases.

The present invention provides agents (e.g., chemotherapeutic agents) that can be released from an implantable device, and which have potent antimicrobial activity at extremely low doses. A wide variety of anti-infective agents can be utilized in combination with a fibrosing agent according to the invention. Discussed in more detail below are several representative examples of agents that can be used: (A) anthracyclines (e.g., doxorubicin and mitoxantrone), (B) fluoropyrimidines (e.g., 5-FU), (C) folic acid antagonists (e.g., methotrexate), (D) podophylotoxins (e.g., etoposide), (E) camptothecins, (F) hydroxyureas, and (G) platinum complexes (e.g., cisplatin).

(A) Anthracyclines

Anthracyclines have the following general structure, where the R groups may be a variety of organic groups:

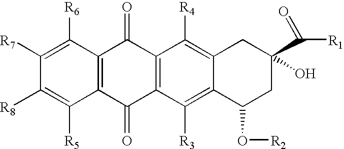

According to U.S. Pat. No. 5,594,158, suitable R groups are as follows: R, is CH₃ or CH₂OH; R₂ is daunosamine or H; R₃ and R₄ are independently one of OH, NO₂, NH₂, F, Cl, Br, I, CN, H or groups derived from these; R₅ is hydrogen, hydroxyl, or methoxy; and R₆₋₈ are all hydrogen. Alternatively, R₅ and R₆ are hydrogen and R₇ and R₈ are alkyl or halogen, or vice versa.

According to U.S. Pat. No. 5,843,903, R₁ may be a conjugated peptide. According to U.S. Pat. No. 4,296,105, R₅ may be an ether linked alkyl group. According to U.S. Pat. No. 4,215,062, R₅ may be OH or an ether linked alkyl group. R₁ may also be linked to the anthracycline ring by a group other than C(O), such as an alkyl or branched alkyl group having the C(O) linking moiety at its end, such as —CH₂CH(CH₂—X)C(O)—R₁, wherein X is H or an alkyl group (see, e.g., U.S. Pat. No. 4,215,062). R₂ may alternately be a group linked by the functional group ═N—NHC(O)—Y, where Y is a group such as a phenyl or substituted phenyl ring. Alternately R₃ may have the following structure: 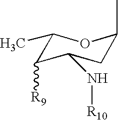
in which R₉ is OH either in or out of the plane of the ring, or is a second sugar moiety such as R₃. R₁₀ may be H or form a secondary amine with a group such as an aromatic group, saturated or partially saturated 5 or 6 membered heterocyclic having at least one ring nitrogen (see U.S. Pat. No. 5,843,903). Alternately, R₁₀ may be derived from an amino acid, having the structure —C(O)CH(NHR₁₁)(R₁₂), in which R₁₁ is H, or forms a C₃₋₄ membered alkylene with R₁₂. R₁₂ may be H, alkyl, aminoalkyl, amino, hydroxyl, mercapto, phenyl, benzyl or methylthio (see U.S. Pat. No. 4,296,105).

Exemplary anthracyclines are doxorubicin, daunorubicin, idarubicin, epirubicin, pirarubicin, zorubicin, and carubicin. Suitable compounds have the structures:

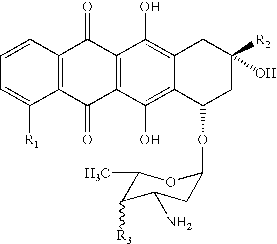

| | R₁ | R₂ | R₃ |
|---|---|---|---|
| Doxorubicin: | OCH₃ | C(O)CH₂OH | OH out of ring |
| | | | plane |
| Epirubicin: | OCH₃ | C(O)CH₂OH | OH in ring plane |
| (4′ epimer of | | | |
| doxorubicin) | | | |
| Daunorubicin: | OCH₃ | C(O)CH₃ | OH out of ring |
| | | | plane |
| Idarubicin: | H | C(O)CH₃ | OH out of ring |
| | | | plane |
| Pirarubicin: | OCH₃ | C(O)CH₂OH | 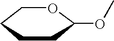 |
| Zorubicin: | OCH₃ | C(CH₃)(═N)NHC(O)C₆H₅ | OH |
| Carubicin: | OH | C(O)CH₃ | OH out of ring |
| | | | plane |

Other suitable anthracyclines are anthramycin, mitoxantrone, menogaril, nogalamycin, aclacinomycin A, olivomycin A, chromomycin A₃, and plicamycin having the structures:

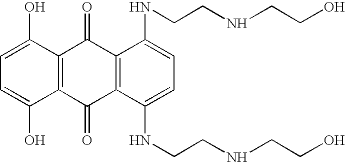

Mitoxantrone

| | R₁ | R₂ | R₃ |
|---|---|---|---|
| Menogaril | H | OCH₃ | H |
| Nogalamycin | O-sugar | H | COOCH₃ |

| | |
|---|---|
| sugar: | 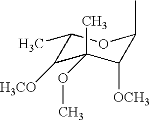 |

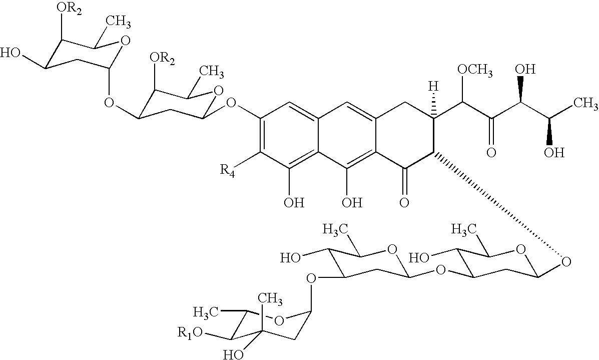

| | R₁ | R₂ | R₃ | R₄ |
|---|---|---|---|---|
| Olivomycin A | COCH(CH₃)₂ | CH₃ | COCH₃ | H |
| Chromomycin A₃ | COCH₃ | CH₃ | COCH₃ | CH₃ |
| Plicamycin | H | H | H | CH₃ |

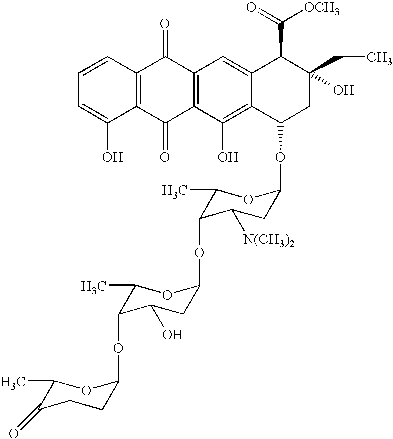

Aclacinomycin A

Other representative anthracyclines include, FCE 23762, a doxorubicin derivative (Quaglia et al., J. Liq. Chromatogr. 17(18): 3911-3923, 1994), annamycin (Zou et al., J. Pharm. Sci. 82(11): 1151-1154, 1993), ruboxyl (Rapoport et al., J. Controlled Release 58(2): 153-162, 1999), anthracycline disaccharide doxorubicin analogue (Pratesi et al., Clin. Cancer Res. 4(11): 2833-2839, 1998), N-(trifluoroacetyl)doxorubicin and 4′-O-acetyl-N-(trifluoroacetyl)doxorubicin (Berube & Lepage, Synth. Commun. 28(6): 1109-1116, 1998), 2-pyrrolinodoxorubicin (Nagy et al., Proc. Nat'l Acad. Sci. U.S.A. 95(4): 1794-1799, 1998), disaccharide doxorubicin analogues (Arcamone et al., J. Nat'l Cancer Inst 89(16): 1217-1223, 1997), 4-demethoxy-7-O-[2,6-dideoxy-4-O-(2,3,6-trideoxy-3-amino-α-L-lyxo-hexopyranosyl)-α-L-lyxo-hexopyranosyl]-adriamicinone doxorubicin disaccharide analogue (Monteagudo et al., Carbohydr. Res. 300(1): 11-16, 1997), 2-pyrrolinodoxorubicin (Nagy et al., Proc. Nat'l Acad. Sci. U.S.A. 94(2): 652-656, 1997), morpholinyl doxorubicin analogues (Duran et al., Cancer Chemother. Pharmacol. 38(3): 210-216, 1996), enaminomalonyl-β-alanine doxorubicin derivatives (Seitz et al., Tetrahedron Lett. 36(9): 1413-16, 1995), cephalosporin doxorubicin derivatives (Vrudhula et al., J. Med. Chem. 38(8): 1380-5, 1995), hydroxyrubicin (Solary et al., Int. J. Cancer 58(1): 85-94, 1994), methoxymorpholino doxorubicin derivative (Kuhl et al., Cancer Chemother. Pharmacol. 33(1): 10-16, 1993), (6-maleimidocaproyl)hydrazone doxorubicin derivative (Wiliner et al., Bioconjugate Chem. 4(6): 521-7, 1993), N-(5,5-diacetoxypent-1-yl) doxorubicin (Cherif & Farquhar, J. Med. Chem. 35(17): 3208-14, 1992), FCE 23762 methoxymorpholinyl doxorubicin derivative (Ripamonti et al., Br. J. Cancer 65(5): 703-7, 1992), N-hydroxysuccinimide ester doxorubicin derivatives (Demant et al., Biochim. Biophys. Acta 1118(1): 83-90, 1991), polydeoxynucleotide doxorubicin derivatives (Ruggiero et al., Biochim. Biophys. Acta 1129(3): 294-302, 1991), morpholinyl doxorubicin derivatives (EPA 434960), mitoxantrone doxorubicin analogue (Krapcho et al., J. Med. Chem. 34(8): 2373-80. 1991), AD198 doxorubicin analogue (Traganos et al., Cancer Res. 51(14): 3682-9, 1991), 4-demethoxy-3′-N-trifluoroacetyldoxorubicin (Horton et al., Drug Des. Delivery 6(2): 123-9, 1990), 4′-epidoxorubicin (Drzewoski et al., Pol. J. Pharmacol. Pharm. 40(2): 159-65, 1988; Weenen et al., Eur. J. Cancer Clin. Oncol. 20(7): 919-26,1984), alkylating cyanomorpholino doxorubicin derivative (Scudder et al., J. Nat'l Cancer Inst. 80(16): 1294-8, 1988), deoxydihydroiodooxorubicin (EPA 275966), adriblastin (Kalishevskaya et al., Vestn. Mosk. Univ., 16(Biol. 1): 21-7, 1988), 4′-deoxydoxorubicin (Schoeizel et al., Leuk. Res. 10(12): 1455-9, 1986), 4-demethyoxy-4′-o-methyldoxorubicin (GIuliani et al., Proc. Int. Congr. Chemother. 16: 285-70-285-77, 1983), 3′-deamino-3′-hydroxydoxorubicin (Horton et al., J. Antibiot. 37(8): 853-8,1984), 4-demethyoxy doxorubicin analogues (Barbieri et al., Drugs Exp. Clin. Res. 10(2): 85-90, 1984), N-L-leucyl doxorubicin derivatives (Trouet et al., Anthracyclines (Proc. Int. Symp. Tumor Pharmacother.), 179-81, 1983), 3′-deamino-3′-(4-methoxy-1-piperidinyl) doxorubicin derivatives (U.S. Pat. No. 4,314,054), 3′-deamino-3′-(4-mortholinyl) doxorubicin derivatives (U.S. Pat. No. 4,301,277), 4′-deoxydoxorubicin and 4′-o-methyldoxorubicin (GIuliani et al., Int. J. Cancer 27(1): 5-13,1981), aglycone doxorubicin derivatives (Chan & Watson, J. Pharm. Sci. 67(12): 1748-52, 1978), SM 5887 (Pharma Japan 1468: 20, 1995), MX-2 (Pharma Japan 1420: 19, 1994), 4′-deoxy-13(S)-dihydro-4′-iododoxorubicin (EP 275966), morpholinyl doxorubicin derivatives (EPA 434960), 3′-deamino-3′-(4-methoxy-1-piperidinyl) doxorubicin derivatives (U.S. Pat. No. 4,314,054), doxorubicin-14-valerate, morpholinodoxorubicin (U.S. Pat. No. 5,004,606), 3′-deamino-3′-(3″-cyano-4″-morpholinyl doxorubicin; 3′-deamino-3′-(3″-cyano-4″-morpholinyl)-13-dihydoxorubicin; (3′-deamino-3′-(3″-cyano-4″-morpholinyl) daunorubicin; 3′-deamino-3′-(3″-cyano-4″-morpholinyl)-3-dihydrodaunorubicin; and 3′-deamino-3′-(4″-morpholinyl-5-iminodoxorubicin and derivatives (U.S. Pat. No. 4,585,859), 3′-deamino-3′-(4-methoxy-1-piperidinyl) doxorubicin derivatives (U.S. Pat. No. 4,314,054) and 3-deamino-3-(4-morpholinyl) doxorubicin derivatives (U.S. Pat. No. 4,301,277).

(B) Fluoropyrimidine Analogues

In another aspect, the therapeutic agent is a fluoropyrimidine analog, such as 5-fluorouracil, or an analogue or derivative thereof, including carmofur, doxifluridine, emitefur, tegafur, and floxuridine. Exemplary compounds have the structures:

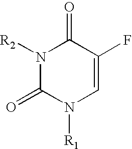

| | R₁ | R₃ |
|---|---|---|
| 5-Fluorouracil | H | H |
| Carmofur | C(O)NH(CH₂)₅CH₃ | H |
| Doxifluridine | A₁ | H |
| Floxuridine | A₂ | H |
| Emitefur | CH₂OCH₂CH₃ | B |
| Tegafur | C | H |

B

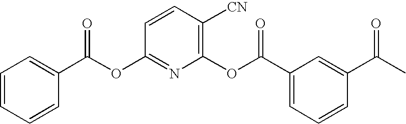

C

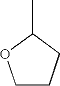

Other suitable fluoropyrimidine analogues include 5-FudR (5-fluoro-deoxyuridine), or an analogue or derivative thereof, including 5-iododeoxyuridine (5-IudR), 5-bromodeoxyuridine (5-BudR), fluorouridine triphosphate (5-FUTP), and fluorodeoxyuridine monophosphate (5-dFUMP). Exemplary compounds have the structures: 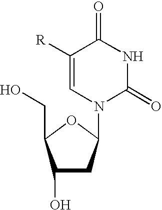
5-Fluoro-2′-deoxyuridine: R═F
5-Bromo-2′-deoxyuridine: R=Br
5-Iodo-2′-deoxyuridine: R=I

Other representative examples of fluoropyrimidine analogues include N3-alkylated analogues of 5-fluorouracil (Kozai et al., J. Chem. Soc., Perkin Trans. 1(19): 3145-3146, 1998), 5-fluorouracil derivatives with 1,4-oxaheteroepane moieties (Gomez et al., Tetrahedron 54(43): 13295-13312, 1998), 5-fluorouracil and nucleoside analogues (Li, Anticancer Res. 17(1A): 21-27, 1997), cis- and trans-5-fluoro-5,6-dihydro-6-alkoxyuracil (Van der Wilt et al., Br. J. Cancer 68(4): 702-7, 1993), cyclopentane 5-fluorouracil analogues (Hronowski & Szarek, Can. J. Chem. 70(4): 1162-9, 1992), A-OT-fluorouracil (Zhang et al., Zongguo Yiyao Gongye Zazhi 20(11): 513-15,1989), N4-trimethoxybenzoyl-5′-deoxy-5-fluorocytidine and 5′-deoxy-5-fluorouridine (Miwa et al., Chem. Pharm. Bull. 38(4): 998-1003, 1990),1-hexylcarbamoyl-5-fluorouracil (Hoshi et al., J. Pharmacobio-Dun. 3(9): 478-81,1980; Maehara et al., Chemotherapy (Basel) 34(6): 484-9, 1988), B-3839 (Prajda et al., In Vivo 2(2): 151-4, 1988), uracil-1-(2-tetrahydrofuryl)-5-fluorouracil (Anai et al., Oncology 45(3): 144-7,1988), 1-(2′-deoxy-2′-fluoro-β-D-arabinofuranosyl)-5-fluorouracil (Suzuko et al., Mol. Pharmacol. 31(3): 301-6, 1987), doxifluridine (Matuura et al., Oyo Yakuri 29(5): 803-31,1985), 5′-deoxy-5-fluorouridine (Bollag & Hartmann, Eur. J. Cancer 16(4): 427-32, 1980), 1-acetyl-3-O-toluyl-5-fluorouracil (Okada, Hiroshima J. Med. Sci. 28(1): 49-66, 1979), 5-fluorouracil-m-formylbenzene-sulfonate (JP 55059173), N′-(2-furanidyl)-5-fluorouracil (JP 53149985) and 1-(2-tetrahydrofuryl)-5-fluorouracil (JP 52089680).

These compounds are believed to function as therapeutic agents by serving as antimetabolites of pyrimidine.

(C) Folic Acid Antagonists

In another aspect, the therapeutic agent is a folic acid antagonist, such as methotrexate or derivatives or analogues thereof, including edatrexate, trimetrexate, raltitrexed, piritrexim, denopterin, tomudex, and pteropterin. Methotrexate analogues have the following general structure: 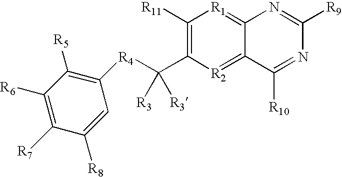
The identity of the R group may be selected from organic groups, particularly those groups set forth in U.S. Pat. Nos. 5,166,149 and 5,382,582. For example, R₁ may be N, R₂ may be N or C(CH₃), R₃ and R₃′ may H or alkyl, e.g., CH₃, R₄ may be a single bond or NR, where R is H or alkyl group. R_{5,6,8} may be H, OCH₃, or alternately they can be halogens or hydro groups. R₇ is a side chain of the general structure: 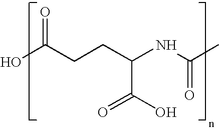
wherein n=1 for methotrexate, n=3 for pteropterin. The carboxyl groups in the side chain may be esterified or form a salt such as a Zn²⁺ salt. R₉ and R₁₀ can be NH₂ or may be alkyl substituted.

Exemplary folic acid antagonist compounds have the structures:

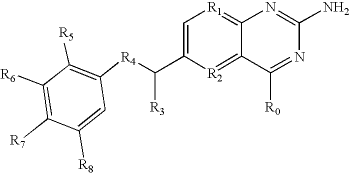

| | R₀ | R₁ | R₂ | R₃ | R₄ | R₅ | R₆ | R₈ | |
|---|---|---|---|---|---|---|---|---|---|
| Methotrexate | NH₂ | N | N | H | N(CH₃) | H | H | A (n = 1) | H |
| Edatrexate | NH₂ | N | N | H | CH(CH₂CH₃) | H | H | A (n = 1) | H |
| Trimetrexate | NH₂ | CH | C(CH₃) | H | NH | H | OCH₃ | OCH₃ | OCH₃ |
| Pteropterin | OH | N | N | H | NH | H | H | A (n = 3) | H |
| Denopterin | OH | N | N | CH3 | N(CH₃) | H | H | A (n = 1) | H |
| Peritrexim | NH₂ | N | C(CH₃) | H | single bond | OCH₃ | H | H | OCH₃ |

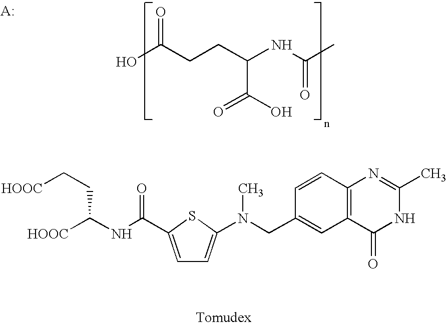

Other representative examples include 6-S-aminoacyloxymethyl mercaptopurine derivatives (Harada et al., Chem. Pharm. Bull. 43(10): 793-6, 1995), 6-mercaptopurine (6-MP) (Kashida et al., Biol. Pharm. Bull. 18(11): 1492-7, 1995), 7,8-polymethyleneimidazo-1,3,2-diazaphosphorines (Nilov et al., Mendeleev Commun. 2: 67, 1995), azathioprine (Chifotides et al., J. Inorg. Biochem. 56(4): 249-64, 1994), methyl-D-glucopyranoside mercaptopurine derivatives (Da Silva et al., Eur. J. Med. Chem. 29(2): 149-52, 1994) and s-alkynyl mercaptopurine derivatives (Ratsino et al., Khim.-Farm. Zh. 15(8): 65-7,1981); indoline ring and a modified ornithine or glutamic acid-bearing methotrexate derivatives (Matsuoka et al., Chem. Pharm. Bull. 45(7): 1146-1150, 1997), alkyl-substituted benzene ring C bearing methotrexate derivatives (Matsuoka et al., Chem. Pharm. Bull. 44(12): 2287-2293, 1996), benzoxazine or benzothiazine moiety-bearing methotrexate derivatives (Matsuoka et al., J. Med. Chem. 40(1): 105-111, 1997), 10-deazaminopterin analogues (DeGraw et al., J. Med. Chem. 40(3): 370-376, 1997), 5-deazaminopterin and 5,10-dideazaminopterin methotrexate analogues (Piper et al., J. Med. Chem. 40(3): 377-384, 1997), indoline moiety-bearing methotrexate derivatives (Matsuoka et al., Chem. Pharm. Bull. 44(7): 1332-1337, 1996), lipophilic amide methotrexate derivatives (Pignatello et al., World Meet. Pharm. Biopharm. Pharm. Technol., 563-4, 1995), L-threo-(2S,4S)-4-fluoroglutamic acid and DL-3,3-difluoroglutamic acid-containing methotrexate analogues (Hart et al., J. Med. Chem. 39(1): 56-65, 1996), methotrexate tetrahydroquinazoline analogue (Gangjee, et al., J. Heterocycl. Chem. 32(1): 243-8, 1995), N-α-aminoacyl)methotrexate derivatives (Cheung et al., Pteridines 3(1-2): 101-2, 1992), biotin methotrexate derivatives (Fan et al., Pteridines 3(1-2): 131-2, 1992), D-glutamic acid or D-erythrou, threo-4-fluoroglutamic acid methotrexate analogues (McGuire et al., Biochem. Pharmacol. 42(12): 2400-3, 1991), β,γ-methano methotrexate analogues (Rosowsky et al., Pteridines 2(3): 133-9, 1991), 10-deazaminopterin (10-EDAM) analogue (Braakhuis et al., Chem. Biol. Pteridines, Proc. Int. Symp. Pteridines Folic Acid Deriv., 1027-30, 1989), γ-tetrazole methotrexate analogue (Kalman et al., Chem. Biol. Pteridines, Proc. Int. Symp. Pteridines Folic Acid Deriv., 1154-7, 1989), N-(L-α-aminoacyl)methotrexate derivatives (Cheung et al., Heterocycles 28(2): 751-8, 1989), meta and ortho isomers of aminopterin (Rosowsky et al., J. Med. Chem. 32(12): 2582, 1989), hydroxymethylmethotrexate (DE 267495), γ-fluoromethotrexate (McGuire et al., Cancer Res. 49(16): 4517-25, 1989), polyglutamyl methotrexate derivatives (Kumar et al., Cancer Res. 46(10): 5020-3, 1986), gem-diphosphonate methotrexate analogues (WO 88/06158), α- and γ-substituted methotrexate analogues (Tsushima et al., Tetrahedron 44(17): 5375-87, 1988), 5-methyl-5-deaza methotrexate analogues (U.S. Pat. No. 4,725,687), Nδ-acyl-Nα-(4-amino-4-deoxypteroyl)-L-ornithine derivatives (Rosowsky et al., J. Med. Chem. 31(7): 1332-7, 1988), 8-deaza methotrexate analogues (Kuehl et al., Cancer Res. 48(6): 1481-8, 1988), acivicin methotrexate analogue (Rosowsky et al., J. Med. Chem. 30(8): 1463-9, 1987), polymeric platinol methotrexate derivative (Carraher et al., Polym. Sci. Technol. (Plenum), 35(Adv. Biomed. Polym.): 311-24, 1987), methotrexate-γ-dimyristoylphophatidylethanolamine (Kinsky et al., Biochim. Biophys. Acta 917(2): 211-18,1987), methotrexate polyglutamate analogues (Rosowsky et al., Chem. Biol. Pteridines, Pteridines Folic Acid Deriv., Proc. Int. Symp. Pteridines Folic Acid Deriv.: Chem., Biol. Clin. Aspects: 985-8, 1986), poly-γ-glutamyl methotrexate derivatives (Kisliuk et al., Chem. Biol. Pteridines, Pteridines Folic Acid Deriv., Proc. Int. Symp. Pteridines Folic Acid Deriv.: Chem., Biol. Clin. Aspects: 989-92, 1986), deoxyuridylate methotrexate derivatives (Webber et al., Chem. Biol. Pteridines, Pteridines Folic Acid Deriv., Proc. Int. Symp. Pteridines Folic Acid Deriv.: Chem., Biol. Clin. Aspects: 659-62, 1986), iodoacetyl lysine methotrexate analogue (Delcamp et al., Chem. Biol. Pteridines, Pteridines Folic Acid Deriv., Proc. Int. Symp. Pteridines Folic Acid Deriv.: Chem., Biol. Clin. Aspects: 807-9, 1986), 2,.omega.-diaminoalkanoid acid-containing methotrexate analogues (McGuire et al., Biochem. Pharmacol. 35(15): 2607-13, 1986), polyglutamate methotrexate derivatives (Kamen & Winick, Methods Enzymol. 122(Vitam. Coenzymes, Pt. G): 339-46, 1986), 5-methyl-5-deaza analogues (Piper et al., J. Med. Chem. 29(6): 1080-7, 1986), quinazoline methotrexate analogue (Mastropaolo et al., J. Med. Chem. 29(1): 155-8, 1986), pyrazine methotrexate analogue (Lever & Vestal, J. Heterocycl. Chem. 22(1): 5-6, 1985), cysteic acid and homocysteic acid methotrexate analogues (U.S. Pat. No. 4,490,529), γ-tert-butyl methotrexate esters (Rosowsky et al., J. Med. Chem. 28(5): 660-7, 1985), fluorinated methotrexate analogues (Tsushima et al., Heterocycles 23(1): 45-9, 1985), folate methotrexate analogue (Trombe, J. Bacteriol. 160(3): 849-53, 1984), phosphonoglutamic acid analogues (Sturtz & Guillamot, Eur. J. Med. Chem.—Chim. Ther. 19(3): 267-73,1984), poly(L-lysine)methotrexate conjugates (Rosowsky et al., J. Med. Chem. 27(7): 888-93, 1984), dilysine and trilysine methotrexate derivates (Forsch & Rosowsky, J. Org. Chem. 49(7): 1305-9, 1984), 7-hydroxymethotrexate (Fabre et al., Cancer Res. 43(10): 4648-52, 1983), poly-γ-glutamyl methotrexate analogues (Piper & Montgomery, Adv. Exp. Med. Biol., 163(Folyl Antifolyl Polyglutamates): 95-100,1983), 3′,5′-dichloromethotrexate (Rosowsky & Yu, J. Med. Chem. 26(10): 1448-52,1983), diazoketone and chloromethylketone methotrexate analogues (Gangjee et al., J. Pharm. Sci. 71(6): 717-19, 1982), 10-propargylaminopterin and alkyl methotrexate homologs (Piper et al., J. Med. Chem. 25(7): 877-80, 1982), lectin derivatives of methotrexate (Lin et al., JNCI 66(3): 523-8, 1981), polyglutamate methotrexate derivatives (Galivan, Mol. Pharmacol. 17(1): 105-10,1980), halogentated methotrexate derivatives (Fox, JNCI 58(4): J955-8, 1977), 8-alkyl-7,8-dihydro analogues (Chaykovsky et al., J. Med. Chem. 20(10): J1323-7, 1977), 7-methyl methotrexate derivatives and dichloromethotrexate (Rosowsky & Chen, J. Med. Chem. 17(12): J1308-11, 1974), lipophilic methotrexate derivatives and 3′,5′-dichloromethotrexate (Rosowsky, J. Med. Chem. 16(10): J1190-3,1973), deaza amethopterin analogues (Montgomery et al., Ann. N.Y. Acad. Sci. 186: J227-34, 1971), MX068 (Pharma Japan, 1658: 18, 1999) and cysteic acid and homocysteic acid methotrexate analogues (EPA 0142220);

These compounds are believed to act as antimetabolites of folic acid.

(D) Podophyllotoxins

In another aspect, the therapeutic agent is a Podophyllotoxin, or a derivative or an analogue thereof. Exemplary compounds of this type are etoposide or teniposide, which have the following structures:

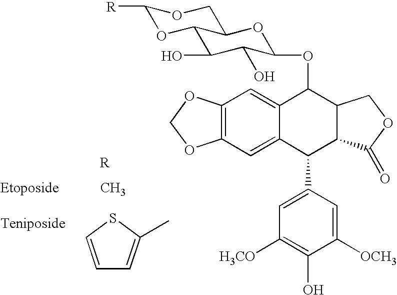

Other representative examples of podophyllotoxins include Cu(II)-VP-16 (etoposide) complex (Tawa et al., Bioorg. Med. Chem. 6(7): 1003-1008, 1998), pyrrolecarboxamidino-bearing etoposide analogues (Ji et al., Bioorg. Med. Chem. Lett. 7(5): 607-612, 1997), 4β-amino etoposide analogues (Hu, University of North Carolina Dissertation, 1992), γ-lactone ring-modified arylamino etoposide analogues (Zhou et al., J. Med. Chem. 37(2): 287-92, 1994), N-glucosyl etoposide analogue (Allevi et al., Tetrahedron Lett. 34(45): 7313-16, 1993), etoposide A-ring analogues (Kadow et al., Bioorg. Med. Chem. Lett. 2(1): 17-22, 1992), 4′-deshydroxy-4′-methyl etoposide (Saulnier et al., Bioorg. Med. Chem. Lett. 2(10): 1213-18, 1992), pendulum ring etoposide analogues (Sinha et al., Eur. J. Cancer 26(5): 590-3, 1990) and E-ring desoxy etoposide analogues (Saulnier et al., J. Med. Chem. 32(7): 1418-20,1989).

These compounds are believed to act as topoisomerase II inhibitors and/or DNA cleaving agents.

(E) Camptothecins

In another aspect, the therapeutic agent is camptothecin, or an analogue or derivative thereof. Camptothecins have the following general structure.

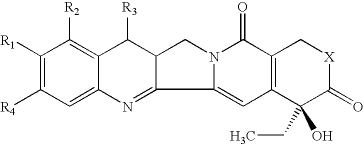

In this structure, X is typically O, but can be other groups, e.g., NH in the case of 21-lactam derivatives. R₁ is typically H or OH, but may be other groups, e.g., a terminally hydroxylated C₁₋₃ alkane. R₂ is typically H or an amino containing group such as (CH₃)₂NHCH₂, but may be other groups e.g., NO₂, NH₂, halogen (as disclosed in, e.g., U.S. Pat. No. 5,552,156) or a short alkane containing these groups. R₃ is typically H or a short alkyl such as C₂H₅. R₄ is typically H but may be other groups, e.g., a methylenedioxy group with R₁.

Exemplary camptothecin compounds include topotecan, irinotecan (CPT-11), 9-aminocamptothecin, 21-lactam-20(S)-camptothecin, 10,11-methylenedioxycamptothecin, SN-38, 9-nitrocamptothecin, 10-hydroxycamptothecin. Exemplary compounds have the structures:

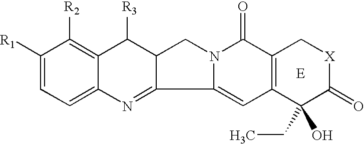

| | R₁ | R₂ | R₃ |
|---|---|---|---|
| Camptothecin: | H | H | H |
| Topotecan: | OH | (CH₃₎₂NHCH₂ | H |
| SN-38: | OH | H | C₂H₅ |

X: O for most analogs, NH for 21-lactam analogs

Camptothecins have the five rings shown here. The ring labeled E must be intact (the lactone rather than carboxylate form) for maximum activity and minimum toxicity.

Camptothecins are believed to function as topoisomerase I inhibitors and/or DNA cleavage agents.

(F) Hydroxyureas

The therapeutic agent of the present invention may be a hydroxyurea. Hydroxyureas have the following general structure:

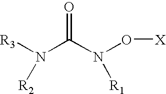

Suitable hydroxyureas are disclosed in, for example, U.S. Pat. No. 6,080,874, wherein R₁ is: 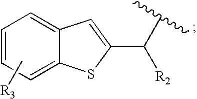
and R₂ is an alkyl group having 1-4 carbons and R₃ is one of H, acyl, methyl, ethyl, and mixtures thereof, such as a methylether.

Other suitable hydroxyureas are disclosed in, e.g., U.S. Pat. No. 5,665,768, wherein R₁ is a cycloalkenyl group, for example N-[3-[5-(4-fluorophenylthio)-furyl]-2-cyclopenten-1-yl]N-hydroxyurea; R₂ is H or an alkyl group having 1 to 4 carbons and R₃ is H; X is H or a cation.

Other suitable hydroxyureas are disclosed in, e.g., U.S. Pat. No. 4,299,778, wherein R₁ is a phenyl group substituted with one or more fluorine atoms; R₂ is a cyclopropyl group; and R₃ and X is H.

Other suitable hydroxyureas are disclosed in, e.g., U.S. Pat. No. 5,066,658, wherein R₂ and R₃ together with the adjacent nitrogen form: 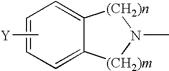
wherein m is 1 or 2, n is 0-2 and Y is an alkyl group.

In one aspect, the hydroxyurea has the structure:

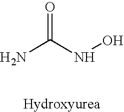

These compounds are thought to function by inhibiting DNA synthesis.

(G) Platinum Complexes

In another aspect, the therapeutic agent is a platinum compound. In general, suitable platinum complexes may be of Pt(II) or Pt(IV) and have this basic structure: 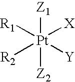
wherein X and Y are anionic leaving groups such as sulfate, phosphate, carboxylate, and halogen; R₁ and R₂ are alkyl, amine, amino alkyl any may be further substituted, and are basically inert or bridging groups. For Pt(II) complexes Z₁ and Z₂ are non-existent. For PT(IV) Z₁ and Z₂ may be anionic groups such as halogen, hydroxy, carboxylate, ester, sulfate or phosphate. See, e.g., U.S. Pat. Nos. 4,588,831 and 4,250,189.

Suitable platinum complexes may contain multiple Pt atoms. See, e.g., U.S. Pat. Nos. 5,409,915 and 5,380,897. For example bisplatinum and triplatinum complexes of the type:

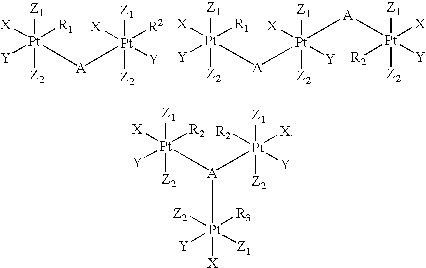

Exemplary platinum compounds are cisplatin, carboplatin, oxaliplatin, and miboplatin having the structures:

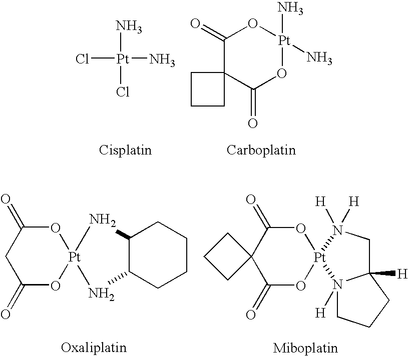

Other representative platinum compounds include (CPA)₂Pt[DOLYM] and (DACH)Pt[DOLYM] cisplatin (Choi et al., Arch. Pharmacal Res. 22(2): 151-156, 1999), Cis-[PtCl₂(4,7-H-5-methyl-7-oxo]1,2,4[triazolo[1,5-a]pyrimidine)₂] (Navarro et al., J. Med. Chem. 41(3): 332-338, 1998), [Pt(cis-1,4-DACH)(trans-Cl₂)(CBDCA)].½MeOH cisplatin (Shamsuddin et al., Inorg. Chem. 36(25): 5969-5971, 1997), 4-pyridoxate diammine hydroxy platinum (Tokunaga et al., Pharm. Sci. 3(7): 353-356, 1997), Pt(II) . . . Pt(II) (Pt₂[NHCHN(C(CH₂)(CH₃))]₄) (Navarro et al., Inorg. Chem. 35(26): 7829-7835, 1996), 254-S cisplatin analogue (Koga et al., Neurol. Res. 18(3): 244-247, 1996), o-phenylenediamine ligand bearing cisplatin analogues (Koeckerbauer & Bednarski, J. Inorg. Biochem. 62(4): 281-298, 1996), trans, cis-[Pt(OAc)₂I₂(en)] (Kratochwil et al., J. Med. Chem. 39(13): 2499-2507, 1996), estrogenic 1,2-diarylethylenediamine ligand (with sulfur-containing amino acids and glutathione) bearing cisplatin analogues (Bednarski, J. Inorg. Biochem. 62(1): 75, 1996), cis-1,4-diaminocyclohexane cisplatin analogues (Shamsuddin et al., J. Inorg. Biochem. 61(4): 291-301, 1996), 5′ orientational isomerof cis-[Pt(NH₃)(4-aminoTEMP-O){d(GpG)}] (Dunham & Lippard, J. Am. Chem. Soc. 117(43): 10702-12, 1995), chelating diamine-bearing cisplatin analogues (Koeckerbauer & Bednarski, J. Pharm. Sci. 84(7): 819-23, 1995), 1,2-diarylethyleneamine ligand-bearing cisplatin analogues (Otto et al., J. Cancer Res. Clin. Oncol. 121(1): 31-8, 1995), (ethylenediamine)platinum(II) complexes (Pasini et al., J. Chem. Soc., Dalton Trans. 4: 579-85, 1995), CI-973 cisplatin analogue (Yang et al., Int. J. Oncol. 5(3): 597-602, 1994), cis-diaminedichloroplatinum(II) and its analogues cis-1,1-cyclobutanedicarbosylato(2R)-2-methyl-1,4-butanediamineplatinum(II) and cis-diammine(glycolato)platinum (Claycamp & Zimbrick, J. Inorg. Biochem. 26(4): 257-67, 1986; Fan et al., Cancer Res. 48(11): 3135-9, 1988; Heiger-Bernays et al., Biochemistry 29(36): 8461-6, 1990; Kikkawa et al., J. Exp. Clin. Cancer Res. 12(4): 233-40, 1993; Murray et al., Biochemistry 31(47): 11812-17, 1992; Takahashi et al., Cancer Chemother. Pharmacol. 33(1): 31-5, 1993), cis-amine-cyclohexylamine-dichloroplatinum(II) (Yoshida et al., Biochem. Pharmacol. 48(4): 793-9, 1994), gem-diphosphonate cisplatin analogues (FR 2683529), (meso-1,2-bis(2,6-dichloro-4-hydroxyplenyl)ethylenediamine) dichloroplatinum(II) (Bednarski et al., J. Med. Chem. 35(23): 4479-85, 1992), cisplatin analogues containing a tethered dansyl group (Hartwig et al., J. Am. Chem. Soc. 114(21): 8292-3, 1992), platinum(II) polyamines (Siegmann et al., Inorg. Met.-Containing Polym. Mater., (Proc. Am. Chem. Soc. Int. Symp.), 335-61, 1990), cis-(3H)dichloro(ethylenediamine)platinum(II) (Eastman, Anal. Biochem. 197(2): 311-15, 1991), trans-diamminedichloroplatinum(II) and cis-(Pt(NH₃)₂(N₃-cytosine)Cl) (Bellon & Lippard, Biophys. Chem. 35(2-3): 179-88, 1990), 3H-cis-1,2-diaminocyclohexanedichloroplatinum(II) and 3H-cis-1,2-diaminocyclohexane-malonatoplatinum (II) (Oswald et al., Res. Commun. Chem. Pathol. Pharmacol. 64(1): 41-58, 1989), diaminocarboxylatoplatinum (EPA 296321), trans-(D,1)-1,2-diaminocyclohexane carrier ligand-bearing platinum analogues (Wyrick & Chaney, J. Labelled Compd. Radiopharm. 25(4): 349-57, 1988), aminoalkylaminoanthraquinone-derived cisplatin analogues (Kitov et al., Eur. J. Med. Chem. 23(4): 381-3, 1988), spiroplatin, carboplatin, iproplatin and JM40 platinum analogues (Schroyen et al., Eur. J. Cancer Clin. Oncol. 24(8): 1309-12, 1988), bidentate tertiary diamine-containing cisplatinum derivatives (Orbell et al., Inorg. Chim. Acta 152(2): 125-34, 1988), platinum(II), platinum(IV) (Liu & Wang, Shandong Yike Daxue Xuebao 24(1): 35-41, 1986), cis-diammine(1,1-cyclobutanedicarboxylato-)platinum(II) (carboplatin, JM8) and ethylenediammine-malonatoplatinum(II) (JM40) (Begg et al., Radiother. Oncol. 9(2): 157-65, 1987), JM8 and JM9 cisplatin analogues (Harstrick et al., Int. J. Androl. 10(1); 139-45, 1987), (NPr4)₂((PtCL4).cis-(PtCl2-(NH2Me)₂)) (Brammer et al., J. Chem. Soc., Chem. Commun. 6: 443-5, 1987), aliphatic tricarboxylic acid platinum complexes (EPA 185225), and cis-dichloro(amino acid)(tert-butylamine)platinum(II) complexes (Pasini & Bersanefti, Inorg. Chim. Acta 107(4): 259-67, 1985). These compounds are thought to function by binding to DNA, i.e., acting as alkylating agents of DNA.

As medical implants are made in a variety of configurations and sizes, the exact dose administered will vary with device size, surface area, design and portions of the implant coated. However, certain principles can be applied in the application of this art. Drug dose can be calculated as a function of dose per unit area (of the portion of the device being coated), total drug dose administered can be measured and appropriate surface concentrations of active drug can be determined. Regardless of the method of application of the drug to the cardiac implant, the preferred anticancer agents, used alone or in combination, should be administered under the following dosing guidelines:
(a) Anthracyclines. Utilizing the anthracycline doxorubicin as an example, whether applied as a polymer coating, incorporated into the polymers which make up the implant components, or applied without a carrier polymer, the total dose of doxorubicin applied to the implant should not exceed 25 mg (range of 0.1 μg to 25 mg). In a particularly preferred embodiment, the total amount of drug applied should be in the range of 1 μg to 5 mg. The dose per unit area (i.e., the amount of drug as a function of the surface area of the portion of the implant to which drug is applied and/or incorporated) should fall within the range of 0.01 μg-100 μg per mm² of surface area. In a particularly preferred embodiment, doxorubicin should be applied to the implant surface at a dose of 0.1 μg/mm²-10 μg/mm². As different polymer and non-polymer coatings will release doxorubicin at differing rates, the above dosing parameters should be utilized in combination with the release rate of the drug from the implant surface such that a minimum concentration of 10⁻⁷-10⁻⁴ M of doxorubicin is maintained on the surface. It is necessary to insure that surface drug concentrations exceed concentrations of doxorubicin known to be lethal to multiple species of bacteria and fungi (i.e., are in excess of 10⁻⁴ M; although for some embodiments lower concentrations are sufficient). In a preferred embodiment, doxorubicin is released from the surface of the implant such that anti-infective activity is maintained for a period ranging from several hours to several months. In a particularly preferred embodiment the drug is released in effective concentrations for a period ranging from 1 week-6 months. It should be readily evident based upon the discussions provided herein that analogues and derivatives of doxorubicin (as described previously) with similar functional activity can be utilized for the purposes of this invention; the above dosing parameters are then adjusted according to the relative potency of the analogue or derivative as compared to the parent compound (e.g., a compound twice as potent as doxorubicin is administered at half the above parameters, a compound half as potent as doxorubicin is administered at twice the above parameters, etc.).

Utilizing mitoxantrone as another example of an anthracycline, whether applied as a polymer coating, incorporated into the polymers which make up the implant, or applied without a carrier polymer, the total dose of mitoxantrone applied should not exceed 5 mg (range of 0.01 μg to 5 mg). In a particularly preferred embodiment, the total amount of drug applied should be in the range of 0.1 μg to 1 mg. The dose per unit area (i.e., the amount of drug as a function of the surface area of the portion of the implant to which drug is applied and/or incorporated) should fall within the range of 0.01 μg-20 μg per mm² of surface area. In a particularly preferred embodiment, mitoxantrone should be applied to the implant surface at a dose of 0.05 μg/mm²-3 μg/mm². As different polymer and non-polymer coatings will release mitoxantrone at differing rates, the above dosing parameters should be utilized in combination with the release rate of the drug from the implant surface such that a minimum concentration of 10⁻⁵-10⁻⁶ M of mitoxantrone is maintained. It is necessary to insure that drug concentrations on the implant surface exceed concentrations of mitoxantrone known to be lethal to multiple species of bacteria and fungi (i.e., are in excess of 10⁻⁵ M; although for some embodiments lower drug levels will be sufficient). In a preferred embodiment, mitoxantrone is released from the surface of the implant such that anti-infective activity is maintained for a period ranging from several hours to several months. In a particularly preferred embodiment the drug is released in effective concentrations for a period ranging from 1 week-6 months. It should be readily evident based upon the discussions provided herein that analogues and derivatives of mitoxantrone (as described previously) with similar functional activity can be utilized for the purposes of this invention; the above dosing parameters are then adjusted according to the relative potency of the analogue or derivative as compared to the parent compound (e.g., a compound twice as potent as mitoxantrone is administered at half the above parameters, a compound half as potent as mitoxantrone is administered at twice the above parameters, etc.).

(b) Fluoropyrimidines Utilizing the fluoropyrimidine 5-fluorouracil as an example, whether applied as a polymer coating, incorporated into the polymers which make up the implant, or applied without a carrier polymer, the total dose of 5-fluorouracil applied should not exceed 250 mg (range of 1.0 μg to 250 mg). In a particularly preferred embodiment, the total amount of drug applied should be in the range of 10 μg to 25 mg. The dose per unit area (i.e., the amount of drug as a function of the surface area of the portion of the implant to which drug is applied and/or incorporated) should fall within the range of 0.1 μg-1 mg per mm² of surface area. In a particularly preferred embodiment, 5-fluorouracil should be applied to the implant surface at a dose of 1.0 μg/mm²-50 μg/mm². As different polymer and non-polymer coatings will release 5-fluorouracil at differing rates, the above dosing parameters should be utilized in combination with the release rate of the drug from the implant surface such that a minimum concentration of 10⁻⁴-10⁻⁷ M of 5-fluorouracil is maintained. It is necessary to insure that surface drug concentrations exceed concentrations of 5-fluorouracil known to be lethal to numerous species of bacteria and fungi (i.e., are in excess of 10⁻⁴ M; although for some embodiments lower drug levels will be sufficient). In a preferred embodiment, 5-fluorouracil is released from the implant surface such that anti-infective activity is maintained for a period ranging from several hours to several months. In a particularly preferred embodiment the drug is released in effective concentrations for a period ranging from 1 week-6 months. It should be readily evident based upon the discussions provided herein that analogues and derivatives of 5-fluorouracil (as described previously) with similar functional activity can be utilized for the purposes of this invention; the above dosing parameters are then adjusted according to the relative potency of the analogue or derivative as compared to the parent compound (e.g., a compound twice as potent as 5-fluorouracil is administered at half the above parameters, a compound half as potent as 5-fluorouracil is administered at twice the above parameters, etc.).

(c) Podophylotoxins Utilizing the podophylotoxin etoposide as an example, whether applied as a polymer coating, incorporated into the polymers which make up the cardiac implant, or applied without a carrier polymer, the total dose of etoposide applied should not exceed 25 mg (range of 0.1 μg to 25 mg). In a particularly preferred embodiment, the total amount of drug applied should be in the range of 1 μg to 5 mg. The dose per unit area (i.e., the amount of drug as a function of the surface area of the portion of the implant to which drug is applied and/or incorporated) should fall within the range of 0.01 μg-100 μg per mm² of surface area. In a particularly preferred embodiment, etoposide should be applied to the implant surface at a dose of 0.1 μg/mm²-10 μg/mm². As different polymer and non-polymer coatings will release etoposide at differing rates, the above dosing parameters should be utilized in combination with the release rate of the drug from the implant surface such that a concentration of 10⁻⁵-10⁻⁶ M of etoposide is maintained. It is necessary to insure that surface drug concentrations exceed concentrations of etoposide known to be lethal to a variety of bacteria and fungi (i.e., are in excess of 10⁻⁵ M; although for some embodiments lower drug levels will be sufficient). In a preferred embodiment, etoposide is released from the surface of the implant such that anti-infective activity is maintained for a period ranging from several hours to several months. In a particularly preferred embodiment the drug is released in effective concentrations for a period ranging from 1 week-6 months. It should be readily evident based upon the discussions provided herein that analogues and derivatives of etoposide (as described previously) with similar functional activity can be utilized for the purposes of this invention; the above dosing parameters are then adjusted according to the relative potency of the analogue or derivative as compared to the parent compound (e.g., a compound twice as potent as etoposide is administered at half the above parameters, a compound half as potent as etoposide is administered at twice the above parameters, etc.).

(d) Combination therapy. It should be readily evident based upon the discussions provided herein that combinations of anthracyclines (e.g., doxorubicin or mitoxantrone), fluoropyrimidines (e.g., 5-fluorouracil), folic acid antagonists (e.g., methotrexate and/or podophylotoxins (e.g., etoposide) can be utilized to enhance the antibacterial activity of the implant coating. Similarly anthracyclines (e.g., doxorubicin or mitoxantrone), fluoropyrimidines (e.g., 5-fluorouracil), folic acid antagonists (e.g., methotrexate and/or podophylotoxins (e.g., etoposide) can be combined with traditional antibiotic and/or antifungal agents to enhance efficacy. The anti-infective agent may be further combined with anti-thrombotic and/or antiplatelet agents (for example, heparin, dextran sulphate, danaparoid, lepirudin, hirudin, AMP, adenosine, 2-chloroadenosine, aspirin, phenylbutazone, indomethacin, meclofenamate, hydrochloroquine, dipyridamole, iloprost, ticlopidine, clopidogrel, abcixamab, eptifibatide, tirofiban, streptokinase, and/or tissue plasminogen activator) to enhance efficacy. In certain embodiments, the fibrosis-inhibiting agent is combined with an agent that can modify metabolism of the agent in vivo to enhance efficacy of the fibrosis-inhibiting agent. One class of therapeutic agents that can be used to alter drug metabolism includes agents capable of inhibiting oxidation of the anti-scarring agent by cytochrome P450 (CYP). In one embodiment, compositions are provided that include a fibrosis-inhibiting agent (e.g., paclitaxel, rapamycin, everolimus) and a CYP inhibitor, which may be combined (e.g., coated) with any of the devices described herein, including, without limitation, stents, grafts, patches, valves, wraps, and films. Representative examples of CYP inhibitors include flavones, azole antifungals, macrolide antibiotics, HIV protease inhibitors, and anti-sense oligomers. Devices comprising a combination of a fibrosis-inhibiting agent and a CYP inhibitor may be used to treat a variety of proliferative conditions that can lead to undesired scarring of tissue, including intimal hyperplasia, surgical adhesions, and tumor growth.

Although the above therapeutic agents have been provided for the purposes of illustration, it should be understood that the present invention is not so limited. For example, although agents are specifically referred to above, the present invention should be understood to include analogues, derivatives and conjugates of such agents. As an illustration, paclitaxel should be understood to refer to not only the common chemically available form of paclitaxel, but analogues (e.g., taxotere, as noted above) and paclitaxel conjugates (e.g., paclitaxel-PEG, paclitaxel-dextran, or paclitaxel-xylos). In addition, to the individual compounds listed above, specif agents that are covalently bound to each other or to another of the described therapeutic agents can also be used for the applications described below. In addition, as will be evident to one of skill in the art, although the agents set forth above may be noted within the context of one class, many of the agents listed in fact have multiple biological activities. Further, more than one therapeutic agent may be utilized at a time (i.e., in combination), or delivered sequentially.

C. Methods for Generating Medical Devices Which Include or Release a Fibrosis-Inhibiting Agent

In the practice of this invention, drug-coated or drug-impregnated implants and medical devices are provided which inhibit fibrosis in and around the device, or prevent “stenosis” of the device/implant in situ, thus enhancing the efficacy. Within various embodiments, fibrosis is inhibited by local or systemic release of specific pharmacological agents that become localized to the tissue adjacent to the device or implant. There are numerous methods available for optimizing delivery of the fibrosis-inhibiting agent to the site of the intervention and several of these are described below.

1) Devices and Implants that Include or Release Fibrosis-Inhibiting Agents

Medical devices or implants of the present invention are coated with, or otherwise adapted to release an agent which inhibits fibrosis on the surface of, or around, the medical device or implant. Medical devices or implants may be adapted to release a fibrosis-inhibiting agent by (a) directly affixing to the implant or device a desired therapeutic agent or composition containing the therapeutic agent (e.g., by either spraying or electrospraying the medical implant with a drug and/or carrier (polymeric or non-polymeric)-drug composition to create a film and/or coating on all, or parts of the internal or external surface of the device; by dipping the implant or device into a drug and/or carrier (polymeric or non-polymeric)-drug solution to coat all or parts of the device or implant; or by other covalent or noncovalent attachment of the therapeutic agent to the device or implant surface); (b) by coating the medical device or implant with a substance such as a hydrogel which either contains or which will in turn absorb the desired fibrosis-inhibiting agent or composition; (c) by interweaving a “thread” composed of, or coated with, the fibrosis-inhibiting agent into the medical implant or device {e.g., a polymeric strand composed of materials that inhibit fibrosis (e.g., paclitaxel, mitoxantrone, doxorubicin, epithilone B, etoposide, TAXOTERE, Tubercidin, vinblastine, geldanamycin, simvastatin, halifuginone, sirolimus, everolimus, mycophenolic acid, 1-alpha-25 dihydroxy vitamin D3, Bay 11-7082, SB202190, sulconizole polymerized drug compositions) or polymers which release a fibrosis-inhibiting agent from the thread}; (d) by covering all, or portions of the device or implant with a sleeve, cover, electrospun fabric or mesh containing a fibrosis-inhibiting agent (i.e., a covering comprised of a fibrosis-inhibiting agent—paclitaxel, mitoxantrone, doxorubicin, epithilone B, etoposide, TAXOTERE, tubercidin, vinblastine, geldanamycin, simvastatin, halifuginone, sirolimus, everolimus, mycophenolic acid, 1-alpha-25 dihydroxy vitamin D₃, Bay 11-7082, SB202190, sulconizole or polymerized compositions containing fibrosis-inhibiting agents); (e) constructing all, or parts of the device or implant itself with the desired agent or composition (e.g., paclitaxel, mitoxantrone, doxorubicin, epithilone B, etoposide, TAXOTERE, tubercidin, vinblastine, geldanamycin, simvastatin, halifuginone, sirolimus, everolimus, mycophenolic acid, 1-alpha-25 dihydroxy vitamin D3, Bay 11-7082, SB202190, sulconizole or polymerized compositions of fibrosis-inhibiting agents); (f) otherwise impregnating the device or implant with the desired fibrosis-inhibiting agent or composition; (g) composing all, or parts, of the device or implant from metal alloys that inhibit fibrosis; (h) constructing all, or parts of the device or implant itself from a degradable or non-degradable polymer that releases one or more fibrosis-inhibiting agents; (i) utilizing specialized multi-drug releasing medical device systems (for example, U.S. Pat. Nos. 6,527,799; 6,293,967; 6,290,673; 6,241,762, U.S. Application Publication Nos. 2003/0199970A1 and 2003/0167085A1, and PCT Publication WO 03/015664) to deliver fibrosis-inhibiting agents alone or in combination.

2) Systemic, Regional and Local Delivery of Fibrosis-Inhibiting Agents

A variety of drug-delivery technologies are available for systemic, regional and local delivery of therapeutic agents. Several of these techniques can be suitable to achieve preferentially elevated levels of fibrosis-inhibiting agents in the vicinity of the medical device or implant, including: (a) using drug-delivery catheters for local, regional or systemic delivery of fibrosis inhibiting agents to the tissue surrounding the device or implant (typically, drug delivery catheters are advanced through the circulation or inserted directly into tissues under radiological guidance until they reach the desired anatomical location; the fibrosis inhibiting agent can then be released from the catheter lumen in high local concentrations in order to deliver therapeutic doses of the drug to the tissue surrounding the device or implant); (b) drug localization techniques such as magnetic, ultrasonic or MRI-guided drug delivery; (c) chemical modification of the fibrosis-inhibiting drug or formulation designed to increase uptake of the agent into damaged tissues (e.g., antibodies directed against damaged or healing tissue components such as macrophages, neutrophils, smooth muscle cells, fibroblasts, extracellular matrix components, neovascular tissue); (d) chemical modification of the fibrosis-inhibiting drug or formulation designed to localize the drug to areas of bleeding or disrupted vasculature; and/or (e) direct injection of the fibrosis-inhibiting agent, for example, under endoscopic vision.

3) Infiltration of Fibrosis-Inhibiting Agents into the Tissue Surrounding a Device or Implant

Alternatively, the tissue cavity into which the device or implant is placed can be treated with a fibrosis-inhibiting agent prior to, during, or after the procedure. This can be accomplished in several ways including: (a) topical application of the fibrosis-inhibiting agent into the anatomical space where the device will be placed (particularly useful for this embodiment is the use of polymeric carriers which release the anti-fibrosing agent over a period ranging from several hours to several weeks. Compositions that can be used for this application include, e.g., fluids, microspheres, pastes, gels, hydrogels, crosslinked gels, microparticulates, sprays, aerosols, solid implants and other formulations which release a fibrosis inhibiting agent into the region where the device or implant will be implanted); (b) microparticulate forms of the therapeutic agent are also useful for directed delivery into the implantation site; (c) sprayable collagen-containing formulations such as COSTASIS (from Angiotech Pharmaceuticals, Inc., Canada), either alone, or loaded with a fibrosis-inhibiting agent, applied to the implantation site (or the implant/device surface); (d) sprayable PEG-containing formulations such as COSEAL (Angiotech Pharmaceuticals, Inc.), SPRAYGEL or DURASEAL (both from Confluent Surgical, Inc., Boston, Mass.), FOCALSEAL (Genzyme Corporation, Cambridge, Mass.), either alone, or loaded with a fibrosis-inhibiting agent, applied to the implantation site (or the implant/device surface); (e) fibrin-containing formulations such as FLOSEAL or TISSEEL (both from Baxter Healthcare Corporation, Fremont, Calif.), either alone, or loaded with a fibrosis-inhibiting agent, applied to the implantation site (or the implant/device surface); (f) hyaluronic acid-containing formulations such as RESTYLANE or PERLANE (both from Q-Med AB, Sweden), HYLAFORM (Inamed Corporation (Santa Barbara, Calif.)), SYNVISC (Biomatrix, Inc., Ridgefield, N.J.), SEPRAFILM or SEPRACOAT (both from Genzyme Corporation, Cambridge, Mass.), INTERGEL (Lifecore Biomedical) loaded with a fibrosis-inhibiting agent applied to the implantation site (or the implant/device surface); (g) polymeric gels for surgical implantation such as REPEL (Life Medical Sciences, Inc., Princeton, N.J.) or FLOGEL (Baxter Healthcare Corporation) loaded with a fibrosis-inhibiting agent applied to the implantation site (or the implant/device surface); (h) orthopedic “cements” used to hold prostheses and tissues in place with a fibrosis-inhibiting agent applied to the implantation site (or the implant/device surface); (i) surgical adhesives containing cyanoacrylates such as DERMABOND (Johnson & Johnson, Inc., New Brunswick, N.J.), INDERMIL (U.S. Surgical Company, Norwalk, Conn.), GLUSTITCH (Blacklock Medical Products Inc., Canada), TISSUMEND II (Veterniary Products Laboratories, Phoenix, Ariz.), VETBOND (3M Company, St. Paul, Minn.), HISTOACRYL BLUE (Davis & Geck, St. Louis, Mo.) and ORABASE SMOOTHE-N-SEAL Liquid Protectant (Colgate-Palmolive Company, New York, N.Y.), either alone, or loaded with a fibrosis-inhibiting agent, applied to the implantation site (or the implant/device surface); (k) surgical implants containing hydroxyapatite, calcium sulfate, tricalcium phosphate, demineralized bone loaded with a fibrosis-inhibiting agent applied to the implantation site (or the implant/device surface);

4) Sustained-Release Preparations of Fibrosis-Inhibiting Agents

As described previously desired fibrosis-inhibiting agents may be admixed with, blended with, conjugated to, or, otherwise modified to contain a polymer composition (which may be either biodegradable or non-biodegradable) or a non-polymeric composition in order to release the therapeutic agent over a prolonged period of time. For many of the aforementioned embodiments, localized delivery as well as localized sustained delivery of the fibrosis inhibiting agent may be required. For example, a desired fibrosis-inhibiting agent may be admixed with, blended with, conjugated to, or, otherwise modified to contain a polymeric composition (which may be either biodegradable or non-biodegradable) or non-polymeric composition in order to release the fibrosis-inhibiting agent over a period of time.

Representative examples of biodegradable polymers suitable for the delivery of fibrosis-inhibiting agents include albumin, collagen, gelatin, hyaluronic acid, starch, cellulose and cellulose derivatives (e.g., methylcellulose, hydroxypropylcellulose, hydroxypropylmethylcellulose, carboxymethylcellulose, cellulose acetate phthalate, cellulose acetate succinate, hydroxypropylmethylcellulose phthalate), casein, dextrans, polysaccharides, fibrinogen, poly(ether ester) multiblock copolymers, based on poly(ethylene glycol) and poly(butylene terephthalate), tyrosine-derived polycarbonates (e.g., U.S. Pat. No. 6,120,491), poly(hydroxyl acids), poly(D,L-lactide), poly(D,L-lactide-co-glycolide), poly(glycolide), poly(hydroxybutyrate), polydioxanone, poly(alkylcarbonate) and poly(orthoesters), polyesters, poly(hydroxyvaleric acid), polydioxanone, degradable polyesters, poly(malic acid), poly(tartronic acid), poly(acrylamides), polyanhydrides, polyphosphazenes, poly(amino acids), poly(alkylene oxide)-poly(ester) block copolymers (e.g., X—Y, X—Y—X or Y—X—Y, R—(Y—X)ₙ, R—(X—Y)ₙ where X is a polyalkylene oxide and Y is a polyester (e.g., polyester can comprise the residues of one or more of the monomers selected from lactide, lactic acid, glycolide, glycolic acid, ε-caprolactone, gamma-caprolactone, hydroxyvaleric acid, hydroxybutyric acid, beta-butyrolactone, gamma-butyrolactone, gamma-valerolactone, ?-decanolactone, d-decanolactone, trimethylene carbonate, 1,4-dioxane-2-one or 1,5-dioxepan-2one.), R is a multifunctional initiator and copolymers as well as blends thereof) and the copolymers as well as blends thereof (see generally, Illum, L., Davids, S. S. (eds.) “Polymers in Controlled Drug Delivery” Wright, Bristol, 1987; Arshady, J. Controlled Release 17: 1-22, 1991; Pitt, Int J. Phar. 59: 173-196, 1990; Holland et al., J. Controlled Release 4: 155-0180, 1986).

Representative examples of non-degradable polymers suitable for the delivery of fibrosis-inhibiting agents include poly(ethylene-co-vinyl acetate) (“EVA”) copolymers, non-degradable polyesters, such as poly(ethylene terephthalate), silicone rubber, acrylic polymers (polyacrylate, polyacrylic acid, polymethylacrylic acid, polymethylmethacrylate, poly(butyl methacrylate)), poly(alkylcynoacrylate) (e.g., poly(ethylcyanoacrylate), poly(butylcyanoacrylate) poly(hexylcyanoacrylate) poly(octylcyanoacrylate)), acrylic resin, polyethylene, polypropylene, polyamides (nylon 6,6), polyurethanes (e.g., CHRONOFLEX AR, CHRONOFLEX AL, BIONATE, and PELLETHANE), poly(ester urethanes), poly(ether urethanes), poly(ester-urea), cellulose esters (e.g., nitrocellulose), polyethers (poly(ethylene oxide), poly(propylene oxide), polyoxyalkylene ether block copolymers based on ethylene oxide and propylene oxide such as the PLURONIC polymers (e.g., F-127 or F87) from BASF Corporation (Mount Olive, N.J.), and poly(tetramethylene glycol), styrene-based polymers (polystyrene, poly(styrene sulfonic acid), poly(styrene)-block-poly(isobutylene)-block-poly(styrene), poly(styrene)-poly(isoprene) block copolymers), and vinyl polymers (polyvinylpyrrolidone, poly(vinyl alcohol), poly(vinyl acetate phthalate) as well as copolymers and blends thereof. Polymers may also be developed which are either anionic (e.g., alginate, carrageenan, carboxymethyl cellulose, poly(acrylamido-2-methyl propane sulfonic acid) and copolymers thereof, poly(methacrylic acid and copolymers thereof and poly(acrylic acid) and copolymers thereof, as well as blends thereof, or cationic (e.g., chitosan, poly-L-lysine, polyethylenimine, and poly(allyl amine)) and blends, copolymers and branched polymers thereof (see generally, Dunn et al., J. Applied Polymer Sci. 50: 353-365, 1993; Cascone et al., J. Materials Sci.: Materials in Medicine 5: 770-774, 1994; Shiraishi et al., Biol. Pharm. Bull. 16(11): 1164-1168, 1993; Thacharodi and Rao, Int'l J. Pharm. 120: 115-118, 1995; Miyazaki et al., Int'l J. Pharm. 118: 257-263, 1995).

Particularly preferred polymeric carriers include poly(ethylene-co-vinyl acetate), polyurethanes (e.g., CHRONOFLEX AR, CHRONOFLEX AL, BIONATE, and PELLETHANE), poly (D,L-lactic acid) oligomers and polymers, poly (L-lactic acid) oligomers and polymers, poly (glycolic acid), copolymers of lactic acid and glycolic acid, poly (caprolactone), poly (valerolactone), polyanhydrides, copolymers of poly (caprolactone) or poly (lactic acid) with a polyethylene glycol (e.g., MePEG), poly(alkylene oxide)-poly(ester) block copolymers (e.g., X—Y, X—Y—X or Y—X—Y, R—(Y—X)ₙ, R—(X—Y)ₙ where X is a polyalkylene oxide and Y is a polyester (e.g., polyester can comprise the residues of one or more of the monomers selected from lactide, lactic acid, glycolide, glycolic acid, e-caprolactone, gamma-caprolactone, hydroxyvaleric acid, hydroxybutyric acid, beta-butyrolactone, gamma-butyrolactone, gamma-valerolactone, ?-decanolactone, d-decanolactone, trimethylene carbonate, 1,4-dioxane-2-one or 1,5-dioxepan-2one.), R is a multifunctional initiator and copolymers as well as blends thereof), nitrocellulose, silicone rubbers, poly(styrene)block-poly(isobutylene)-block-poly(styrene), poly(acrylate) polymers and blends, admixtures, or co-polymers of any of the above. Other preferred polymers include collagen, poly(alkylene oxide)-based polymers, polysaccharides such as hyaluronic acid, chitosan and fucans, and copolymers of polysaccharides with degradable polymers, as well as blends thereof.

Other representative polymers capable of sustained localized delivery of fibrosis-inhibiting agents include carboxylic polymers, polyacetates, polycarbonates, polyethers, polyethylenes, polyvinylbutyrals, polysilanes, polyureas, polyoxides, polystyrenes, polysulfides, polysulfones, polysulfonides, polyvinylhalides, pyrrolidones, rubbers, thermal-setting polymers, cross-linkable acrylic and methacrylic polymers, ethylene acrylic acid copolymers, styrene acrylic copolymers, vinyl acetate polymers and copolymers, vinyl acetal polymers and copolymers, epoxies, melamines, other amino resins, phenolic polymers, and copolymers thereof, water-insoluble cellulose ester polymers (including cellulose acetate propionate, cellulose acetate, cellulose acetate butyrate, cellulose nitrate, cellulose acetate phthalate, and mixtures thereof), polyvinylpyrrolidone, polyethylene glycols, polyethylene oxide, polyvinyl alcohol, polyethers, polysaccharides, hydrophilic polyurethane, polyhydroxyacrylate, dextran, xanthan, hydroxypropyl cellulose, and homopolymers and copolymers of N-vinylpyrrolidone, N-vinyllactam, N-vinyl butyrolactam, N-vinyl caprolactam, other vinyl compounds having polar pendant groups, acrylate and methacrylate having hydrophilic esterifying groups, hydroxyacrylate, and acrylic acid, and combinations thereof; cellulose esters and ethers, ethyl cellulose, hydroxyethyl cellulose, cellulose nitrate, cellulose acetate, cellulose acetate butyrate, cellulose acetate propionate, natural and synthetic elastomers, rubber, acetal, styrene polybutadiene, acrylic resin, polyvinylidene chloride, polycarbonate, homopolymers and copolymers of vinyl compounds, polyvinylchloride, and polyvinylchloride acetate.

Representative examples of patents relating to drug-delivery polymers and the preparation include PCT Publication Nos. WO 98/19713, WO 01/17575, WO 01/41821, WO 01/41822, and WO 01/15526 (as well as the corresponding U.S. applications); U.S. Pat. Nos. 4,500,676, 4,582,865, 4,629,623, 4,636,524, 4,713,448, 4,795,741, 4,913,743, 5,069,899, 5,099,013, 5,128,326, 5,143,724, 5,153,174, 5,246,698, 5,266,563, 5,399,351, 5,525,348, 5,800,412, 5,837,226, 5,942,555, 5,997,517, 6,007,833, 6,071,447, 6,090,995, 6,106,473, 6,110,483, 6,121,027, 6,156,345, 6,214,901, 6,368,611 6,630,155, 6,528,080, RE37,950, 6,46,1631, 6,143,314, 5,990,194, 5,792,469, 5,780,044, 5,759,563, 5,744,153, 5,739,176, 5,733,950, 5,681,873, 5,599,552, 5,340,849, 5,278,202, 5,278,201, 6,589,549, 6,287,588, 6,201,072, 6,117,949, 6,004,573, 5,702,717, 6,413,539, 5,714,159, 5,612,052; and U.S. Patent Application Publication Nos. 2003/0068377, 2002/0192286, 2002/0076441, and 2002/0090398.

In one embodiment, all or a portion of the device is coated with a primer (bonding) layer and a drug release layer, as described in U.S. patent application entitled, “Stent with Medicated Multi-Layer Hybrid Polymer Coating,” filed Sep. 16, 2003 (U.S. Ser. No. 10/662,877).

In order to develop a hybrid polymer delivery system for targeted therapy, it is desirable to be able to control and manipulate the properties of the system both in terms of physical and drug release characteristics. The active agents can be imbibed into a surface hybrid polymer layer, or incorporated directly into the hybrid polymer coating solutions. Imbibing drugs into surface polymer layers is an efficient method for evaluating polymer-drug performance in the laboratory, but for commercial production it may be preferred for the polymer and drug to be premixed in the casting mixture. Greater efficacy can be achieved by combining the two elements in the coating mixtures in order to control the ratio of active agent to polymer in the coatings. Such ratios are important parameters to the final properties of the medicated layers, i.e., they allow for better control of active agent concentration and duration of pharmacological activity.

Typical polymers used in the drug-release system can include water-insoluble cellulose esters, various polyurethane polymers including hydrophilic and hydrophobic versions, hydrophilic polymers such as polyethylene glycol (PEG), polyethylene oxide (PEO), polyvinylpyrrolidone (PVP), PVP copolymers such as vinyl acetate, hydroxyethyl methacrylate (HEMA) and copolymers such as methylmethacrylate (PMMA-HEMA), and other hydrophilic and hydrophobic acrylate polymers and copolymers containing functional groups such as carboxyl and/or hydroxyl.

Cellulose esters such as cellulose acetate, cellulose acetate propionate, cellulose acetate butyrate, cellulose acetate phthalate, and cellulose nitrate may be used. In one aspect of the invention, the therapeutic agent is formulated with a cellulose ester. Cellulose nitrate is a preferred cellulose ester because of its compatibility with the active agents and its ability to impart non-tackiness and cohesiveness to the coatings. Cellulose nitrate has been shown to stabilize entrapped drugs in ambient and processing conditions. Various grades of cellulose nitrate are available and may be used in a coating on a device, including cellulose nitrate having a nitrogen content=11.8-12.2%. Various viscosity grades, including 3.5, 0.5 or 0.25 seconds, may be used in order to provide proper Theological properties when combined with the coating solids used in these formulations. Higher or lower viscosity grades can be used. However, the higher viscosity grades can be more difficult to use because of their higher viscosities. Thus, the lower viscosity grades, such as 3.5, 0.5 or 0.25 seconds, are generally preferred. Physical properties such as tensile strength, elongation, flexibility, and softening point are related to viscosity (molecular weight) and can decrease with the lower molecular weight species, especially below the 0.25 second grades.

The cellulose derivatives comprise hydroglucose structures. Cellulose nitrate is a hydrophobic, water-insoluble polymer, and has high water resistance properties. This structure leads to high compatibility with many active agents, accounting for the high degree of stabilization provided to drugs entrapped in cellulose nitrate. The structure of nitrocellulose is given below:

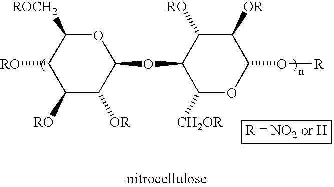

Cellulose nitrate is a hard, relatively inflexible polymer, and has limited adhesion to many polymers that are typically used to make medical devices. Also, control of drug elution dynamics is limited if only one polymer is used in the binding matrix. Accordingly, in one embodiment of the invention, the therapeutic agent is formulated with two or more polymers before being associated with the device. In one aspect, the agent is formulated with both polyurethane ((e.g., CHRONOFLEX AR, CHRONOFLEX AL, and BIONATE, PELLETHANE) and cellulose nitrate to provide a hybrid polymer drug loaded matrix. Polyurethanes provide the hybrid polymer matrix with greater flexibility and adhesion to the device, particularly when the device has been pre-coated with a primer. Polyurethanes can also be used to slow or hasten the drug elution from coatings. Aliphatic, aromatic, polytetramethylene ether glycol, and polycarbonate are among the types of polyurethanes, which can be used in the coatings. In one aspect, an anti-scarring agent (e.g., paclitaxel) may be incorporated into a carrier that includes a polyurethane and a cellulose derivative. A heparin complex, such as benzalkonium heparinate or tridodecylammonium heparinate), may optionally be included in the formulation.

From the structure below, it is possible to see how more or less hydrophilic polyurethane polymers may be created based on the number of hydrophilic groups contained in the polymer structures. In one aspect of the invention, the device is associated with a formulation that includes therapeutic agent, cellulose ester, and a polyurethane that is water-insoluble, flexible, and compatible with the cellulose ester.

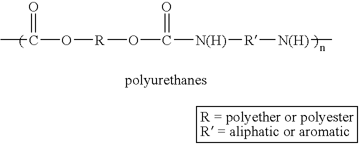

Polyvinylpyrrolidone (PVP) is a polyamide that possesses unusual complexing and colloidal properties and is essentially physiologically inert. PVP and other hydrophilic polymers are typically biocompatible. PVP may be incorporated into drug loaded hybrid polymer compositions in order to increase drug release rates. In one embodiment, the concentration of PVP that is used in drug loaded hybrid polymer compositions can be less than 20%. This concentration can not make the layers bioerodable or lubricious. In general, PVP concentrations from <1% to greater than 80% are deemed workable. In one aspect of the invention, the therapeutic agent that is associated with an device is formulated with a PVP polymer.

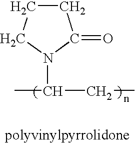

Acrylate polymers and copolymers including polymethylmethacrylate (PMMA) and polymethylmethacrylate hydroxyethyl methacrylate (PMMA/HEMA) are known for their biocompatibility as a result of their widespread use in contact and intraocular lens applications. This class of polymer generally provokes very little smooth muscle and endothelial cell growth, and very low inflammatory response (Bar). These polymers/copolymers are compatible with drugs and the other polymers and layers of the instant invention. Thus, in one aspect, the device is associated with a composition that comprises an anti-scarring agent as described above, and an acrylate polymer or copolymer.

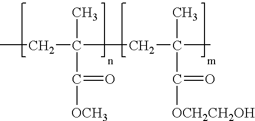

### Methylmethacrylate Hydroxyethylmethacrylate Copolymer

It should be obvious to one of skill in the art that the polymers as described herein can also be blended or copolymerized in various compositions as required to deliver therapeutic doses of fibrosis-inhibiting agents.

Polymeric carriers for fibrosis-inhibiting agents can be fashioned in a variety of forms, with desired release characteristics and/or with specific properties depending upon the device, composition or implant being utilized. For example, polymeric carriers may be fashioned to release a fibrosis-inhibiting agent upon exposure to a specific triggering event such as pH (see, e.g., Heller et al., “Chemically Self-Regulated Drug Delivery Systems,” in Polymers in Medicine III, Elsevier Science Publishers B.V., Amsterdam, 1988, pp. 175-188; Kang et al., J. Applied Polymer Sci. 48: 343-354, 1993; Dong et al., J. Controlled Release 19: 171-178, 1992; Dong and Hoffman, J. Controlled Release 15: 141-152, 1991; Kim et al., J. Controlled Release 28: 143-152, 1994; Cornejo-Bravo et al., J. Controlled Release 33: 223-229, 1995; Wu and Lee, Pharm. Res. 10(10): 1544-1547, 1993; Serres et al., Pharm. Res. 13(2): 196-201, 1996; Peppas, “Fundamentals of pH- and Temperature-Sensitive Delivery Systems,” in Gurny et al. (eds.), Pulsatile Drug Delivery, Wissenschaftliche Verlagsgesellschaft mbH, Stuttgart, 1993, pp. 41-55; Doelker, “Cellulose Derivatives,” 1993, in Peppas and Langer (eds.), Biopolymers I, Springer-Verlag, Berlin). Representative examples of pH-sensitive polymers include poly (acrylic acid) and its derivatives (including for example, homopolymers such as poly(aminocarboxylic acid); poly(acrylic acid); poly(methyl acrylic acid), copolymers of such homopolymers, and copolymers of poly(acrylic acid) and/or acrylate or acrylamide Imonomers such as those discussed above. Other pH sensitive polymers include polysaccharides such as cellulose acetate phthalate; hydroxypropylmethylcellulose phthalate; hydroxypropylmethylcellulose acetate succinate; cellulose acetate trimellilate; and chitosan. Yet other pH sensitive polymers include any mixture of a pH sensitive polymer and a water-soluble polymer.

Likewise, fibrosis-inhibiting agents can be delivered via polymeric carriers which are temperature sensitive (see, e.g., Chen et al., “Novel Hydrogels of a Temperature-Sensitive PLURONIC Grafted to a Bioadhesive Polyacrylic Acid Backbone for Vaginal Drug Delivery,” in Proceed. Intern. Symp. Control. Rel. Bioact. Mater. 22: 167-168, Controlled Release Society, Inc., 1995; Okano, “Molecular Design of Stimuli-Responsive Hydrogels for Temporal Controlled Drug Delivery,” in Proceed. Intern. Symp. Control. Rel. Bioact. Mater. 22: 111-112, Controlled Release Society, Inc., 1995; Johnston et al., Pharm. Res. 9(3): 425-433, 1992; Tung, Int'l J. Pharm. 107: 85-90, 1994; Harsh and Gehrke, J. Controlled Release 17: 175-186, 1991; Bae et al., Pharm. Res. 8(4): 531-537, 1991; Dinarvand and D′Emanuele, J. Controlled Release 36: 221-227, 1995; Yu and Grainger, “Novel Thermo-sensitive Amphiphilic Gels: Poly N-isopropylacrylamide-co-sodium acrylate-co-n-N-alkylacrylamide Network Synthesis and Physicochemical Characterization,” Dept. of Chemical & Biological Sci., Oregon Graduate Institute of Science & Technology, Beaverton, Oreg., pp. 820-821; Zhou and Smid, “Physical Hydrogels of Associative Star Polymers,” Polymer Research Institute, Dept. of Chemistry, College of Environmental Science and Forestry, State Univ. of New York, Syracuse, N.Y., pp. 822-823; Hoffman et al., “Characterizing Pore Sizes and Water ‘Structure’ in Stimuli-Responsive Hydrogels,” Center for Bioengineering, Univ. of Washington, Seattle, Wash., p. 828; Yu and Grainger, “Thermo-sensitive Swelling Behavior in Crosslinked N-isopropylacrylamide Networks: Cationic, Anionic and Ampholytic Hydrogels,” Dept. of Chemical & Biological Sci., Oregon Graduate Institute of Science & Technology, Beaverton, Oreg., pp. 829-830; Kim et al., Pharm. Res. 9(3): 283-290, 1992; Bae et al., Pharm. Res. 8(5): 624-628, 1991; Kono et al., J. Controlled Release 30:69-75, 1994; Yoshida et al., J. Controlled Release 32: 97-102, 1994; Okano et al., J. Controlled Release 36: 125-133, 1995; Chun and Kim, J. Controlled Release 38: 39-47, 1996; D'Emanuele and Dinarvand, Int'l J. Pharm. 118: 237-242, 1995; Katono et al., J. Controlled Release 16: 215-228, 1991; Hoffman, “Thermally Reversible Hydrogels Containing Biologically Active Species,” in Migliaresi et al. (eds.), Polymers in Medicine III, Elsevier Science Publishers B.V., Amsterdam, 1988, pp. 161-167; Hoffman, “Applications of Thermally Reversible Polymers and Hydrogels in Therapeutics and Diagnostics,” in Third International Symposium on Recent Advances in Drug Delivery Systems, Salt Lake City, Utah, Feb. 24-27,1987, pp. 297-305; Gutowska et al., J. Controlled Release 22: 95-104, 1992; Palasis and Gehrke, J. Controlled Release 18: 1-12, 1992; Paavola et al., Pharm. Res. 12(12): 1997-2002, 1995).

Representative examples of thermogelling polymers, and the gelatin temperature (LCST (° C.)) include homopolymers such as poly(N-methyl-N-n-propylacrylamide), 19.8; poly(N-n-propylacrylamide), 21.5; poly(N-methyl-N-isopropylacrylamide), 22.3; poly(N-n-propylmethacrylamide), 28.0; poly(N-isopropylacrylamide), 30.9; poly(N, n-diethylacrylamide), 32.0; poly(N-isopropylmethacrylamide), 44.0; poly(N-cyclopropylacrylamide), 45.5; poly(N-ethylmethyacrylamide), 50.0; poly(N-methyl-N-ethylacrylamide), 56.0; poly(N-cyclopropylmethacrylamide), 59.0; poly(N-ethylacrylamide), 72.0. Moreover thermogelling polymers may be made by preparing copolymers between (among) monomers of the above, or by combining such homopolymers with other water-soluble polymers such as acrylmonomers (e.g., acrylic acid and derivatives thereof, such as methylacrylic acid, acrylate monomers and derivatives thereof, such as butyl methacrylate, butyl acrylate, lauryl acrylate, and acrylamide monomers and derivatives thereof, such as N-butyl acrylamide and acrylamide).

Other representative examples of thermogelling polymers include cellulose ether derivatives such as hydroxypropyl cellulose, 41° C.; methyl cellulose, 55° C.; hydroxypropylmethyl cellulose, 66° C.; and ethylhydroxyethyl cellulose, polyalkylene oxide-polyester block copolymers of the structure X—Y, Y—X—Y and X—Y—X where X in a polyalkylene oxide and Y is a biodegradable polyester (e.g., PLG-PEG-PLG) and PLURONICs such as F-127, 10-15° C.; L-122, 19° C.; L-92, 26° C.; L-81, 20° C.; and L-61, 24° C.

Representative examples of patents relating to thermally gelling polymers and the preparation include U.S. Pat. Nos. 6,451,346; 6,201,072; 6,117,949; 6,004,573; 5,702,717; and 5,484,610; and PCT Publication Nos. WO 99/07343; WO 99/18142; WO 03/17972; WO 01/82970; WO 00/18821; WO 97/15287; WO 01/41735; WO 00/00222 and WO 00/38651.

Fibrosis-inhibiting agents may be linked by occlusion in the matrices of the polymer, bound by covalent linkages, or encapsulated in microcapsules. Within certain embodiments of the invention, therapeutic compositions are provided in non-capsular formulations such as microspheres (ranging from nanometers to micrometers in size), pastes, threads of various size, films, or sprays. In one aspect, the anti-scarring agent may be incorporated into biodegradable magnetic nanospheres. The nanospheres may be used, for example, to replenish an anti-scarring agent into an implanted intravascular device, such as a stent containing a weak magnetic alloy (see, e.g., Z. Forbes, B. B. Yellen, G. Friedman, K. Barbee. “An approach to targeted drug delivery based on uniform magnetic fields,” IEEE Trans. Magn. 39(5): 3372-3377 (2003)).

Within certain aspects of the present invention, therapeutic compositions may be fashioned in the form of microspheres, microparticles and/or nanoparticles having any size ranging from about 30 nm to 500 μm, depending upon the particular use. These compositions can be formed by spray-drying methods, milling methods, coacervation methods, W/O emulsion methods, W/O/W emulsion methods, and solvent evaporation methods. In other aspects, these compositions can include microemulsions, emulsions, liposomes and micelles. Alternatively, such compositions may also be readily applied as a “spray”, which solidifies into a film or coating for use as a device/implant surface coating or to line the tissues of the implantation site. Such sprays may be prepared from microspheres of a wide array of sizes, including for example, from 0.1 μm to 3 μm, from 10 μm to 30 μm, and from 30 μm to 100 μm.

Therapeutic compositions of the present invention may also be prepared in a variety of “paste” or gel forms. For example, within one embodiment of the invention, therapeutic compositions are provided which are liquid at one temperature (e.g., temperature greater than 37° C., such as 40° C., 45° C., 50° C., 55° C. or 60° C.), and solid or semi-solid at another temperature (e.g., ambient body temperature, or any temperature lower than 37° C.). Such “thermopastes” may be readily made utilizing a variety of techniques (see, e.g., PCT Publication WO 98/24427). Other pastes may be applied as a liquid, which solidify in vivo due to dissolution of a water-soluble component of the paste and precipitation of encapsulated drug into the aqueous body environment. These “pastes” and “gels” containing fibrosis-inhibiting agents are particularly useful for application to the surface of tissues that will be in contact with the implant or device.

Within yet other aspects of the invention, the therapeutic compositions of the present invention may be formed as a film or tube. These films or tubes can be porous or non-porous. Preferably, such films or tubes are generally less than 5, 4, 3, 2, or 1 mm thick, more preferably less than 0.75 mm, 0.5 mm, 0.25 mm, or, 0.10 mm thick. Films or tubes can also be generated of thicknesses less than 50 μm, 25 μm or 10 μm. Such films are preferably flexible with a good tensile strength (e.g., greater than 50, preferably greater than 100, and more preferably greater than 150 or 200 N/cm²), good adhesive properties (i.e., adheres to moist or wet surfaces), and have controlled permeability. Fibrosis-inhibiting agents contained in polymeric films are particularly useful for application to the surface of a device or implant as well as to the surface of tissue, cavity or an organ.

Within further aspects of the present invention, polymeric carriers are provided which are adapted to contain and release a hydrophobic fibrosis-inhibiting compound, and/or the carrier containing the hydrophobic compound in combination with a carbohydrate, protein or polypeptide. Within certain embodiments, the polymeric carrier contains or comprises regions, pockets, or granules of one or more hydrophobic compounds. For example, within one embodiment of the invention, hydrophobic compounds may be incorporated within a matrix which contains the hydrophobic fibrosis-inhibiting compound, followed by incorporation of the matrix within the polymeric carrier. A variety of matrices can be utilized in this regard, including for example, carbohydrates and polysaccharides such as starch, cellulose, dextran, methylcellulose, sodium alginate, heparin, chitosan and hyaluronic acid, proteins or polypeptides such as albumin, collagen and gelatin. Within alternative embodiments, hydrophobic compounds may be contained within a hydrophobic core, and this core contained within a hydrophilic shell.

Other carriers that may likewise be utilized to contain and deliver fibrosis-inhibiting fibrosis-inhibiting agents described herein include: hydroxypropyl cyclodextrin (Cserhati and Hollo, Int. J. Pharm. 108: 69-75, 1994), liposomes (see, e.g., Sharma et al., Cancer Res. 53: 5877-5881, 1993; Sharma and Straubinger, Pharm. Res. 11(60): 889-896, 1994; WO 93/18751; U.S. Pat. No. 5,242,073), liposome/gel (WO 94/26254), nanocapsules (Bartoli et al., J. Microencapsulation 7(2): 191-197, 1990), micelles (Alkan-Onyuksel et al., Pharm. Res. 11(2): 206-212, 1994), implants (Jampel et al., Invest Ophthalm. Vis. Science 34(11): 3076-3083, 1993; Walter et al., Cancer Res. 54: 22017-2212, 1994), nanoparticles (Violante and Lanzafame PAACR), nanoparticles—modified (U.S. Pat. No. 5,145,684), nanoparticles (surface modified) (U.S. Pat. No. 5,399,363), micelle (surfactant) (U.S. Pat. No. 5,403,858), synthetic phospholipid compounds (U.S. Pat. No. 4,534,899), gas borne dispersion (U.S. Pat. No. 5,301,664), liquid emulsions, foam, spray, gel, lotion, cream, ointment, dispersed vesicles, particles or droplets solid- or liquid-aerosols, microemulsions (U.S. Pat. No. 5,330,756), polymeric shell (nano- and micro-capsule) (U.S. Pat. No. 5,439,686), emulsion (Tarr et al., Pharm Res. 4: 62-165, 1987), nanospheres (Hagan et al., Proc. Intern. Symp. Control Rel. Bioact. Mater. 22, 1995; Kwon et al., Pharm Res. 12(2): 192-195; Kwon et al., Pharm Res. 10(7): 970-974; Yokoyama et al., J. Contr. Rel. 32: 269-277, 1994; Gref et al., Science 263: 1600-1603, 1994; Bazile et al., J. Pharm. Sci. 84: 493-498, 1994) and implants (U.S. Pat. No. 4,882,168).

Within another aspect of the present invention, polymeric carriers can be materials that are formed in situ. In one embodiment, the precursors can be monomers or macromers that contain unsaturated groups that can be polymerized and/or cross-linkeds. The monomers or macromers can then, for example, be injected into the treatment area or onto the surface of the treatment area and polymerized in situ using a radiation source (e.g., visible or UV light) or a free radical system (e.g., potassium persulfate and ascorbic acid or iron and hydrogen peroxide). The polymerization step can be performed immediately prior to, simultaneously to or post injection of the reagents into the treatment site. Representative examples of compositions that undergo free radical polymerization reactions are described in WO 01/44307, WO 01/68720, WO 02/072166, WO 03/043552, WO 93/17669, WO 00/64977; U.S. Pat. Nos. 5,900,245, 6,051,248, 6,083,524, 6,177,095, 6,201,065, 6,217,894, 6,639,014, 6,352,710, 6,410,645, 6,531,147, 5,567,435, 5,986,043, 6,602,975; U.S. Patent Application Publication Nos. 2002/012796A1, 2002/0127266A1, 2002/0151650A1, 2003/0104032A1, 2002/0091229A1, and 2003/0059906A1.

In another embodiment, the reagents can undergo an electrophilic-nucleophilic reaction to produce a crosslinked matrix. For example, a 4-armed thiol derivatized polyethylene glycol can be reacted with a 4 armed NHS-derivatized polyethylene glycol under basic conditions (pH>about 8). Representative examples of compositions that undergo electrophilic-nucleophilic crosslinking reactions are described in U.S. Pat. Nos. 5,752,974; 5,807,581; 5,874,500; 5,936,035; 6,051,648; 6,165,489; 6,312,725; 6,458,889; 6,495,127; 6,534,591; 6,624,245; 6,566,406; 6,610,033; 6,632,457; PCT Application Published Nos. WO 04/060405 and WO 04/060346. Other examples of in situ forming materials that can be used include those based on the crosslinking of proteins (described in U.S. Pat. Nos. RE38158; 4,839,345; 5,514,379, 5,583,114; 6,458,147; 6,371,975; U.S. Publication Nos 2002/0161399; 2001/0018598 and PCT Publication Nos. WO 03/090683; WO 01/45761; WO 99/66964 and WO 96/03159).

As described above, the anti-fibrosing agent can be associated with a medical device using the polymeric carriers or coatings described above. In addition to the compositions and methods described above, there are various other compositions and methods that are known in the art. Representative examples of these compositions and methods for applying (e.g., coating) these compositons to devices are described in U.S. Pat. Nos. 6,610,016; 6,358,557; 6,306,176; 6,110,483; 6,106,473; 5,997,517; 5,800,412; 5,525,348; 5,331,027; 5,001,009; 6,562,136; 6,406,754; 6,344,035; 6,254,921; 6,214,901; 6,077,698; 6,603,040; 6,278,018; 6,238,799; 6,096,726, 5,766,158, 5,599,576, 4,119,094; 4,100,309; 6,599,558; 6,369,168; 6,521,283; 6,497,916; 6,251,964; 6,225,431; 6,087,462; 6,083,257; 5,739,237; 5,739,236; 5,705,583; 5,648,442; 5,645,883; 5,556,710; 5,496,581; 4,689,386; 6,214,115; 6,090,901; 6,599,448; 6,054,504; 4,987,182; 4,847,324; and 4,642,267; U.S. Patent Application Publication Nos. 2002/0146581, 2003/0129130, 2003/0129130, 2001/0026834; 2003/0190420; 2001/0000785; 2003/0059631; 2003/0190405; 2002/0146581; 2003/020399; 2001/0026834; 2003/0190420; 2001/0000785; 2003/0059631; 2003/0190405; and 2003/020399; and PCT Publication Nos. WO 02/055121; WO 01/57048; WO 01/52915; and WO 01/01957.

Within another aspect of the invention, the biologically active agent can be delivered with a non-polymeric agent. These non-polymeric carriers can include sucrose derivatives (e.g., sucrose acetate isobutyrate, sucrose oleate), sterols such as cholesterol, stigmasterol, β-sitosterol, and estradiol; cholesteryl esters such as cholesteryl stearate; C₁₂-C₂₄ fatty acids such as lauric acid, myristic acid, palmitic acid, stearic acid, arachidic acid, behenic acid, and lignoceric acid; C₁₈-C₃₆ mono-, di- and triacylglycerides such as glyceryl monooleate, glyceryl monolinoleate, glyceryl monolaurate, glyceryl monodocosanoate, glyceryl monomyristate, glyceryl monodicenoate, glyceryl dipalmitate, glyceryl didocosanoate, glyceryl dimyristate, glyceryl didecenoate, glyceryl tridocosanoate, glyceryl trimyristate, glyceryl tridecenoate, glycerol tristearate and mixtures thereof; sucrose fatty acid esters such as sucrose distearate and sucrose palmitate; sorbitan fatty acid esters such as sorbitan monostearate, sorbitan monopalmitate and sorbitan tristearate; C₁₆-C₁₈ fatty alcohols such as cetyl alcohol, myristyl alcohol, stearyl alcohol, and cetostearyl alcohol; esters of fatty alcohols and fatty acids such as cetyl palmitate and cetearyl palmitate; anhydrides of fatty acids such as stearic anhydride; phospholipids including phosphatidylcholine (lecithin), phosphatidylserine, phosphatidylethanolamine, phosphatidylinositol, and lysoderivatives thereof; sphingosine and derivatives thereof; spingomyelins such as stearyl, palmitoyl, and tricosanyl spingomyelins; ceramides such as stearyl and palmitoyl ceramides; glycosphingolipids; lanolin and lanolin alcohols, calcium phosphate, sintered and unscintered hydoxyapatite, zeolites; and combinations and mixtures thereof.

Representative examples of patents relating to non-polymeric delivery systems and the preparation include U.S. Pat. Nos. 5,736,152; 5,888,533; 6,120,789; 5,968,542; and 5,747,058.

The fibrosis-inhibiting agent may be delivered as a solution. The fibrosis-inhibiting agent can be incorporated directly into the solution to provide a homogeneous solution or dispersion. In certain embodiments, the solution is an aqueous solution. The aqueous solution may futher include buffer salts, as well as viscosity modifying agents (e.g., hyaluronic acid, alginates, carboxymethylcelluloe (CMC), and the like). In another aspect of the invention, the solution can include a biocompatible solvent, such as ethanol, DMSO, glycerol, PEG-200, PEG-300 or NMP.

Within another aspect of the invention, the fibrosis-inhibiting agent can further comprise a secondary carrier. The secondary carrier can be in the form of microspheres (e.g., PLGA, PLLA, PDLLA, PCL, gelatin, polydioxanone, poly(alkylcyanoacrylate)), nanospheres (PLGA, PLLA, PDLLA, PCL, gelatin, polydioxanone, poly(alkylcyanoacrylate)), liposomes, emulsions, microemulsions, micelles (SDS, block copolymers of the form X—Y, X—Y—X or Y—X—Y, R—(Y—X)ₙ, R—(X—Y)ₙ where X is a polyalkylene oxide (e.g., poly(ethylene oxide, poly(propylene oxide, block copolymers of poly(ethylene oxide) and poly(propylene oxide) and Y is a polyester (e.g., polyester can comprise the residues of one or more of the monomers selected from lactide, lactic acid, glycolide, glycolic acid, e-caprolactone, gamma-caprolactone, hydroxyvaleric acid, hydroxybutyric acid, beta-butyrolactone, gamma-butyrolactone, gamma-valerolactone, ?-decanolactone, d-decanolactone, trimethylene carbonate, 1,4-dioxane-2-one or 1,5-dioxepan-2one.), R is a multifunctional initiator and copolymers as well as blends thereof.), zeolites or cyclodextrins.

Within another aspect of the invention, these fibrosis-inhibiting agent/secondary carrier compositions can be a) incorporated directly into or onto the device, b) incorporated into a solution, c) incorporated into a gel or viscous solution, d) incorporated into the composition used for coating the device or e) incorporated into or onto the device following coating of the device with a coating composition.

For example, fibrosis-inhibiting agent loaded PLGA microspheres can be incorporated into a polyurethane coating solution which is then coated onto the device.

In yet another example, the device can be coated with a polyurethane and then allowed to partially dry such that the surface is still tacky. A particulate form of the fibrosis-inhibiting agent or fibrosis-inhibiting agent/secondary carrier can then be applied to all or a portion of the tacky coating after which the device is dried.

In yet another example, the device can be coated with one of the coatings described above. A thermal treatment process can then be used to soften the coating, after which the fibrosis-inhibiting agent or the fibrosis-inhibiting agent/secondary carrier is applied to the entire device or to a portion of the device (e.g., outer surface).

Within another aspect of the invention, the coated device which inhibits or reduces an in vivo fibrotic reaction is further coated with a compound or compositions which delay the release of and/or activity of the fibrosis-inhibiting agent. Representative examples of such agents include biologically inert materials such as gelatin, PLGA/MePEG film, PLA, polyurethanes, silicone rubbers, surfactants, lipids, or polyethylene glycol, as well as biologically active materials such as heparin (e.g., to induce coagulation).

For example, in one embodiment of the invention, the active agent on the device is top-coated with a physical barrier. Such barriers can include non-degradable materials or biodegradable materials such as gelatin, PLGA/MePEG film, PLA, or polyethylene glycol among others. In one embodiment, the rate of diffusion of the therapeutic agent in the barrier coat is slower that the rate of diffusion of the therapeutic agent in the coating layer. In the case of PLGA/MePEG, once the PLGA/MePEG becomes exposed to the bloodstream, the MePEG can dissolve out of the PLGA, leaving channels through the PLGA layer to an underlying layer containing the fibrosis-inhibiting agent, which then can then diffuse into the vessel wall and initiate its biological activity.

In another embodiment of the invention, a particulate form of the active agent may be coated onto the stent (or any of the devices described below) using a polymer (e.g., PLG, PLA, aor a polyurethane). A second polymer, that dissolves slowly or degrades (e.g., MePEG-PLGA or PLG) and that does not contain the active agent, may be coated over the first layer. Once the top layer dissolves or degrades, it exposes the under coating which allows the active agent to be exposed to the treatment site or to be released from the coating.

Within another aspect of the invention, the outer layer of the coating of a coated device, which inhibits an in vivo fibrotic response, is further treated to crosslink the outer layer of the coating. This can be accomplished by subjecting the coated device to a plasma treatment process. The degree of crosslinking and nature of the surface modification can be altered by changing the RF power setting, the location with respect to the plasma, the duration of treatment as well as the gas composition introduced into the plasma chamber.

Protection of a biologically active surface can also be utilized by coating the device surface with an inert molecule that prevents access to the active site through steric hindrance, or by coating the surface with an inactive form of the fibrosis-inhibiting agent, which is later activated. For example, the device can be coated with an enzyme, which causes either release of the fibrosis-inhibiting agent or activates the fibrosis-inhibiting agent.

In another embodiment, the device is coated with a fibrosis-inhibiting agent and then further coated with a composition that comprises an anticoagulant such as heparin. As the anticoagulant dissolves away, the anticoagulant activity slows or stops, and the newly exposed fibrosis-inhibiting agent is available to inhibit or reduce fibrosis from occurring in the adjacent tissue.

The device can be coated with an inactive form of the fibrosis-inhibiting agent, which is then activated once the device is deployed. Such activation can be achieved by injecting another material into the treatment area after the device (as desribed below) is deployed or after the fibrosis-inhibiting agent has been administered to the treatment area (via, e.g., injections, spray, wash, drug delivery catheters or balloons). For example, the device can be coated with an inactive form of the fibrosis-inhibiting agent. Once the device is deployed, the activating substance is injected or applied into or onto the treatment site where the inactive form of the fibrosis-inhibiting agent has been applied. For example, a device can be coated with a biologically active fibrosis-inhibiting agent and a first substance having moieties that capable of forming an ester bond with another material. The coating can be covered with a second substance such as polyethylene glycol. The first and second substances can react to form an ester bond via, e.g., a condensation reaction. Prior to the deployment of the device, an esterase is injected into the treatment site around the outside of the device, which can cleave the bond between the ester and the fibrosis-inhibiting agent, allowing the agent to initiate fibrosis-inhibition.

In another aspect, a medical device may include a plurality of reservoirs within its structure, each reservoir configured to house and protect a therapeutic drug. The reservoirs may be formed from divets in the device surface or micropores or channels in the device body. In one aspect, the reservoirs are formed from voids in the structure of the device. The reservoirs may house a single type of drug or more than one type of drug. The drug(s) may be formulated with a carrier (e.g., a polymeric or non-polymeric material) that is loaded into the reservoirs. The filled reservoir can function as a drug delivery depot which can release drug over a period of time dependent on the release kinetics of the drug from the carrier. In certain embodiments, the reservoir may be loaded with a plurality of layers. Each layer may include a different drug having a particular amount (dose) of drug, and each layer may have a different composition to further tailor the amount of drug that is released from the substrate. The multi-layered carrier may further include a barrier layer that prevents release of the drug(s). The barrier layer can be used, for example, to control the direction that the drug elutes from the void.

Within certain embodiments of the invention, the therapeutic compositions may also comprise additional ingredients such as surfactants (e.g., PLURONICS, such as F-127, L-122, L-101, L-92, L-81, and L-61), anti-inflammatory agents (e.g., dexamethasone or asprin), anti-thrombotic agents (e.g., heparin, high activity heparin, heparin quaternary amine complexes (e.g., heparin benzalkonium chloride complex)), anti-infective agents (e.g., 5-fluorouracil, triclosan, rifamycim, and silver compounds), preservatives, anti-oxidants and/or anti-platelet agents.

Within certain embodiments of the invention, the therapeutic agent or carrier can also comprise radio-opaque, echogenic materials and magnetic resonance imaging (MRI) responsive materials (i.e., MRI contrast agents) to aid in visualization of the device under ultrasound, fluoroscopy and/or MRI. For example, a device may be made with or coated with a composition which is echogenic or radiopaque (e.g., made with echogenic or radiopaque with materials such as powdered tantalum, tungsten, barium carbonate, bismuth oxide, barium sulfate, metrazimide, iopamidol, iohexol, iopromide, iobitridol, iomeprol, iopentol, ioversol, ioxilan, iodixanol, iotrolan, acetrizoic acid derivatives, diatrizoic acid derivatives, iothalamic acid derivatives, ioxithalamic acid derivatives, metrizoic acid derivatives, iodamide, lypophylic agents, iodipamide and ioglycamic acid or, by the addition of microspheres or bubbles which present an acoustic interface). Visualization of a device by ultrasonic imaging may be achieved using an echogenic coating. Echogenic coatings are described in, e.g., U.S. Pat. Nos. 6,106,473 and 6,610,016. For visualization under MRI, contrast agents (e.g., gadolinium (III) chelates or iron oxide compounds) may be incorporated into or onto the device, such as, for example, as a component in a coating or within the void volume of the device (e.g., within a lumen, reservoir, or within the structural material used to form the device). In some embodiments, a medical device may include radio-opaque or MRI visible markers (e.g., bands) that may be used to orient and guide the device during the implantation procedure.

In another embodiment, these agents can be contained within the same coating layer as the therapeutic agent or they may be contained in a coating layer (as described above) that is either applied before or after the therapeutic agent containing layer.

Medical implants may, alternatively, or in addition, be visualized under visible light, using fluorescence, or by other spectroscopic means. Visualization agents that can be included for this purpose include dyes, pigments, and other colored agents. In one aspect, the medical implant may further include a colorant to improve visualization of the implant in vivo and/or ex vivo. Frequently, implants can be difficult to visualize upon insertion, especially at the margins of implant. A coloring agent can be incorporated into a medical implant to reduce or eliminate the incidence or severity of this problem. The coloring agent provides a unique color, increased contrast, or unique fluorescence characteristics to the device. In one aspect, a solid implant is provided that includes a colorant such that it is readily visible (under visible light or using a fluorescence technique) and easily differentiated from its implant site. In another aspect, a colorant can be included in a liquid or semi-solid composition. For example, a single component of a two component mixture may be colored, such that when combined ex-vivo or in-vivo, the mixture is sufficiently colored.

The coloring agent may be, for example, an endogenous compound (e.g., an amino acid or vitamin) or a nutrient or food material and may be a hydrophobic or a hydrophilic compound. Preferably, the colorant has a very low or no toxicity at the concentration used. Also preferred are colorants that are safe and normally enter the body through absorption such as β-carotene. Representative examples of colored nutrients (under visible light) include fat soluble vitamins such as Vitamin A (yellow); water soluble vitamins such as Vitamin B12 (pink-red) and folic acid (yellow-orange); carotenoids such as β-carotene (yellow-purple) and lycopene (red). Other examples of coloring agents include natural product (berry and fruit) extracts such as anthrocyanin (purple) and saffron extract (dark red). The coloring agent may be a fluorescent or phosphorescent compound such as α-tocopherolquinol (a Vitamin E derivative) or L-tryptophan. Derivatives, analogues, and isomers of any of the above colored compound also may be used. The method for incorporating a colorant into an implant or therapeutic composition may be varied depending on the properties of and the desired location for the colorant. For example, a hydrophobic colorant may be selected for hydrophobic matrices. The colorant may be incorporated into a carrier matrix, such as micelles. Further, the pH of the environment may be controlled to further control the color and intensity.

In one aspect, the composition of the present invention include one or more coloring agents, also referred to as dyestuffs, which will be present in an effective amount to impart observable coloration to the composition, e.g., the gel. Examples of coloring agents include dyes suitable for food such as those known as F.D. & C. dyes and natural coloring agents such as grape skin extract, beet red powder, beta carotene, annato, carmine, turmeric, paprika, and so forth. Derivatives, analogues, and isomers of any of the above colored compound also may be used. The method for incorporating a colorant into an implant or therapeutic composition may be varied depending on the properties of and the desired location for the colorant. For example, a hydrophobic colorant may be selected for hydrophobic matrices. The colorant may be incorporated into a carrier matrix, such as micelles. Further, the pH of the environment may be controlled to further control the color and intensity.

In one aspect, the compositions of the present invention include one or more preservatives or bacteriostatic agents, present in an effective amount to preserve the composition and/or inhibit bacterial growth in the composition, for example, bismuth tribromophenate, methyl hydroxybenzoate, bacitracin, ethyl hydroxybenzoate, propyl hydroxybenzoate, erythromycin, 5-fluorouracil, methotrexate, doxorubicin, mitoxantrone, rifamycin, chlorocresol, benzalkonium chlorides, and the like. Examples of the preservative include paraoxybenzoic acid esters, chlorobutanol, benzylalcohol, phenethyl alcohol, dehydroacetic acid, sorbic acid, etc. In one aspect, the compositions of the present invention include one or more bactericidal (also known as bacteriacidal) agents.

In one aspect, the compositions of the present invention include one or more antioxidants, present in an effective amount. Examples of the antioxidant include sulfites, alpha-tocopherol and ascorbic acid.

Within certain aspects of the present invention, the therapeutic composition should be biocompatible, and release one or more fibrosis-inhibiting agents over a period of several hours, days, or, months. As described above, “release of an agent” refers to any statistically significant presence of the agent, or a subcomponent thereof, which has disassociated from the compositions and/or remains active on the surface of (or within) the composition. The compositions of the present invention may release the anti-scarring agent at one or more phases, the one or more phases having similar or different performance (e.g., release) profiles. The therapeutic agent may be made available to the tissue at amounts which may be sustainable, intermittent, or continuous; in one or more phases; and/or rates of delivery; effective to reduce or inhibit any one or more components of fibrosis (or scarring), including: formation of new blood vessels (angiogenesis), migration and proliferation of connective tissue cells (such as fibroblasts or smooth muscle cells), deposition of extracellular matrix (ECM), and remodeling (maturation and organization of the fibrous tissue).

Thus, release rate may be programmed to impact fibrosis (or scarring) by releasing an anti-scarring agent at a time such that at least one of the components of fibrosis is inhibited or reduced. Moreover, the predetermined release rate may reduce agent loading and/or concentration as well as potentially providing minimal drug washout and thus, increases efficiency of drug effect. Any one of the at least one anti-scarring agents may perform one or more functions, including inhibiting the formation of new blood vessels (angiogenesis), inhibiting the migration and proliferation of connective tissue cells (such as fibroblasts or smooth muscle cells), inhibiting the deposition of extracellular matrix (ECM), and inhibiting remodeling (maturation and organization of the fibrous tissue). In one embodiment, the rate of release may provide a sustainable level of the anti-scarring agent to the susceptible tissue site. In another embodiment, the rate of release is substantially constant. The rate may decrease and/or increase over time, and it may optionally include a substantially non-release period. The release rate may comprise a plurality of rates. In an embodiment, the plurality of release rates may include rates selected from the group consisting of substantially constant, decreasing, increasing, substantially non-releasing.

The total amount of anti-scarring agent made available on, in or near the device may be in an amount ranging from about 0.01 μg (micrograms) to about 2500 mg (milligrams). Generally, the anti-scarring agent may be in the amount ranging from 0.01 μg to about 10 μg; or from 10 μg to about 1 mg; or from 1 mg to about 10 mg; or from 10 mg to about 100 mg; or from 100 mg to about 500 mg; or from 500 mg to about 2500 mg.

The total surface amount of anti-scarring agent on, in or near the device may be in an amount ranging from less than 0.01 μg to about 2500 μg per mm² of device surface area. Generally, the anti-scarring agent may be in the amount ranging from less than 0.01 μg; or from 0.01 μg to about 10 μg; or from 10 μg to about 250 μg; or from 250 μg to about 2500 μg,

The anti-scarring agent that is on, in or near the device may be released from the composition in a time period that may be measured from the time of implantation, which ranges from about less than 1 day to about 180 days. Generally, the release time may also be from about less than 1 day to about 7 days; from 7 days to about 14 days; from 14 days to about 28 days; from 28 days to about 56 days; from 56 days to about 90 days; from 90 days to about 180 days.

The amount of anti-scarring agent released from the composition as a function of time may be determined based on the in vitro release characteristics of the agent from the composition. The in vitro release rate may be determined by placing the anti-scarring agent within the composition or device in an appropriate buffer such as 0.1M phosphate buffer (pH 7.4)) at 37° C. Samples of the buffer solution are then periodically removed for analysis by HPLC, and the buffer is replaced to avoid any saturation effects.

Based on the in vitro release rates, the release of anti-scarring agent per day may range from an amount ranging from about 0.01 μg (micrograms) to about 2500 mg (milligrams). Generally, the anti-scarring agent that may be released in a day may be in the amount ranging from 0.01 μg to about 10 μg; or from 10 μg to about 1 mg; or from 1 mg to about 10 mg; or from 10 mg to about 100 mg; or from 100 mg to about 500 mg; or from 500 mg to about 2500 mg.

In one embodiment, the anti-scarring agent is made available to the susceptible tissue site in a programmed, sustained, and/or controlled manner which results in increased efficiency and/or efficacy. Further, the release rates may vary during either or both of the initial and subsequent release phases. There may also be additional phase(s) for release of the same substance(s) and/or different substance(s).

Further, therapeutic compositions and devices of the present invention should preferably be have a stable shelf-life for several months and capable of being produced and maintained under sterile conditions. Many pharmaceuticals are manufactured to be sterile and this criterion is defined by the USP XXII <1211>. The term “USP” refers to U.S. Pharmacopeia (see www.usp.org, Rockville, Md.). Sterilization may be accomplished by a number of means accepted in the industry and listed in the USP XXII <1211>, including gas sterilization, ionizing radiation or, when appropriate, filtration. Sterilization may be maintained by what is termed asceptic processing, defined also in USP XXII <1211>. Acceptable gases used for gas sterilization include ethylene oxide. Acceptable radiation types used for ionizing radiation methods include gamma, for instance from a cobalt 60 source and electron beam. A typical dose of gamma radiation is 2.5 MRad. Filtration may be accomplished using a filter with suitable pore size, for example 0.22 μm and of a suitable material, for instance polytetrafluoroethylene (e.g., TEFLON from E.I. DuPont De Nemours and Company, Wilmington, Del.).

In another aspect, the compositions and devices of the present invention are contained in a container that allows them to be used for their intended purpose, i.e., as a pharmaceutical composition. Properties of the container that are important are a volume of empty space to allow for the addition of a constitution medium, such as water or other aqueous medium, e.g., saline, acceptable light transmission characteristics in order to prevent light energy from damaging the composition in the container (refer to USP XXII <661>), an acceptable limit of extractables within the container material (refer to USP XXII), an acceptable barrier capacity for moisture (refer to USP XXII <671>) or oxygen. In the case of oxygen penetration, this may be controlled by including in the container, a positive pressure of an inert gas, such as high purity nitrogen, or a noble gas, such as argon.

Typical materials used to make containers for pharmaceuticals include USP Type I through III and Type NP glass (refer to USP XXII <661>), polyethylene, TEFLON, silicone, and gray-butyl rubber.

In one embodiment, the product containers can be thermoformed plastics. In another embodiment, a seconday package can be used for the product. In another embodiment, product can be in a sterile container that is placed in a box that is labeled to describe the contents of the box.

5) Coating of Devices with Fibrosis-Inhibiting Agents

As described above, a range of polymeric and non-polymeric materials can be used to incorporate the fibrosis-inhibiting agent onto or into a device. The anti-fibrosing agent composition can be incorporated into or onto the device in a variety of ways. Coating of the device with the fibrosis-inhibiting agent containing composition or with the fibrosis-inhibiting agent only is one process that can be used to incorporate the fibrosis-inhibiting agent into or onto the device. The anti-fibrosing agent or anti-fibrosing composition may be coated onto the entire device or a portion of the device using a method, such as by dipping, spraying, painting or vacuum deposition, that is appropriate for the particular type of device.

a) Dip coating

Dip coating is one coating process that can be used. In one embodiment, the fibrosis-inhibiting agent is dissolved in a solvent for the fibrosis agent and is then coated onto the device.

Fibrosis-Inhibiting Agent with an Inert-Solvent

In one embodiment, the solvent is an inert solvent for the device such that the solvent does not dissolve the medical device to any great extent and is not absorbed by the device to any great extent. The device can be immersed, either partially or completely, in the fibrosis-inhibiting agent/solvent solution for a specific period of time. The rate of immersion into the fibrosis-inhibiting agent/solvent solution can be altered (e.g., 0.001 cm per sec to 50 cm per sec). The device can then be removed from the solution. The rate at which the device can be withdrawn from the solution can be altered (e.g., 0.001 cm per sec to 50 cm per sec). The coated device can be air-dried. The dipping process can be repeated one or more times depending on the specific application. The device can be dried under vacuum to reduce residual solvent levels. This process will result in the fibrosis-inhibiting agent being coated on the surface of the device.

Fibrosis-Inhibiting Agent with a Swelling Solvent

In one embodiment, the solvent is one that will not dissolve the device but will be absorbed by the device. These solvents can thus swell the device to some extent. The device can be immersed, either partially or completely, in the fibrosis-inhibiting agent/solvent solution for a specific period of time (seconds to days). The rate of immersion into the fibrosis-inhibiting agent/solvent solution can be altered (e.g., 0.001 cm per sec to 50 cm per sec). The device can then be removed from the solution. The rate at which the device can be withdrawn from the solution can be altered (e.g., 0.001 cm per sec to 50 cm per sec). The coated device can be air-dried. The dipping process can be repeated one or more times depending on the specific application. The device can be dried under vacuum to reduce residual solvent levels. This process will result in the fibrosis-inhibiting agent being adsorbed into the medical device. The fibrosis-inhibiting agent may also be present on the surface of the device. The amount of surface associated fibrosis-inhibiting agent may be reduced by dipping the coated device into a solvent for the fibrosis-inhibiting agent or by spraying the coated device with a solvent for the fibrosis-inhibiting agent.

Fibrosis-Inhibiting Agent with a Solvent

In one embodiment, the solvent is one that will be absorbed by the device and that will dissolve the device. The device can be immersed, either partially or completely, in the fibrosis-inhibiting agent/solvent solution for a specific period of time (seconds to hours). The rate of immersion into the fibrosis-inhibiting agent/solvent solution can be altered (e.g., 0.001 cm per sec to 50 cm per sec). The device can then be removed from the solution. The rate at which the device can be withdrawn from the solution can be altered (e.g., 0.001 cm per sec to 50 cm per sec). The coated device can be air-dried. The dipping process can be repeated one or more times depending on the specific application. The device can be dried under vacuum to reduce residual solvent levels. This process will result in the fibrosis-inhibiting agent being adsorbed into the medical device as well as being surface associated. In the preferred embodiment, the exposure time of the device to the solvent can be such that there are no significant permanent dimensional changes to the device. The fibrosis-inhibiting agent may also be present on the surface of the device. The amount of surface associated fibrosis-inhibiting agent may be reduced by dipping the coated device into a solvent for the fibrosis-inhibiting agent or by spraying the coated device with a solvent for the fibrosis-inhibiting agent.

In the above description the device can be a device that has not been modified as well as a device that has been further modified by coating with a polymer, surface treated by plasma treatment, flame treatment, corona treatment, surface oxidation or reduction, surface etching, mechanical smoothing or roughening, or grafting prior to the coating process.

In one embodiment, the fibrosis-inhibiting agent and a polymer are dissolved in a solvent, for both the polymer and the fibrosis-inhibiting agent, and are then coated onto the device.

In any one the above dip coating methods, the surface of the device can be treated with a plasma polymerization method prior to coating of the scarring agent or scarring agent containing composition, such that a thin polymeric layer is deposited onto the device surface. Examples of such methods include parylene coating of devices and the use of various monomers such hydrocyclosiloxane monomers. Parylene coating may be especially advantageous if the device, or portions of the device, is composed of materials (e.g., stainless steel, nitinol) that do not allow incorporation of the therapeutic agent(s) into the surface layer using one of the above methods. A parylene primer layer may be deposited onto the device using a parylene coater (e.g., PDS 2010 LABCOTER2 from Cookson Electronics) and a suitable reagent (e.g., di-p-xylylene or dichloro-di-p-xylylene) as the coating feed material. Parylene compounds are commercially available, for example, from Specialty Coating Systems, Indianapolis, Ind.), including PARYLENE N (di-p-xylylene), PARYLENE C (a monchlorinated derivative of PARYLENE N, and PARYLENE D, a dichlorinated derivative of PARYLENE N).

Fibrosis-Inhibiting Agent/Polymer with an Inert-Solvent

In one embodiment, the solvent is an inert solvent for the device such that the solvent does not dissolve the medical device to any great extent and is not absorbed by the device to any great extent. The device can be immersed, either partially or completely, in the fibrosis-inhibiting agent/polymer/solvent solution for a specific period of time. The rate of immersion into the fibrosis-inhibiting agent/polymer/solvent solution can be altered (e.g., 0.001 cm per sec to 50 cm per sec). The device can then be removed from the solution. The rate at which the device can be withdrawn from the solution can be altered (e.g., 0.001 cm per sec to 50 cm per sec). The coated device can be air-dried. The dipping process can be repeated one or more times depending on the specific application. The device can be dried under vacuum to reduce residual solvent levels. This process will result in the fibrosis-inhibiting agent/polymer being coated on the surface of the device.

Fibrosis-Inhibiting Agent/Polymer with a Swelling Solvent

In one embodiment, the solvent is one that will not dissolve the device but will be absorbed by the device. These solvents can thus swell the device to some extent. The device can be immersed, either partially or completely, in the fibrosis-inhibiting agent/polymer/solvent solution for a specific period of time (seconds to days). The rate of immersion into the fibrosis-inhibiting agent/polymer/solvent solution can be altered (e.g., 0.001 cm per sec to 50 cm per sec). The device can then be removed from the solution. The rate at which the device can be withdrawn from the solution can be altered (e.g., 0.001 cm per sec to 50 cm per sec). The coated device can be air-dried. The dipping process can be repeated one or more times depending on the specific application. The device can be dried under vacuum to reduce residual solvent levels. This process will result in the fibrosis-inhibiting agent/polymer being coated onto the surface of the device as well as the potential for the fibrosis-inhibiting agent being adsorbed into the medical device. The fibrosis-inhibiting agent may also be present on the surface of the device. The amount of surface associated fibrosis-inhibiting agent may be reduced by dipping the coated device into a solvent for the fibrosis-inhibiting agent or by spraying the coated device with a solvent for the fibrosis-inhibiting agent.

Fibrosis-Inhibiting Agent/Polymer with a Solvent

In one embodiment, the solvent is one that will be absorbed by the device and that will dissolve the device. The device can be immersed, either partially or completely, in the fibrosis-inhibiting agent/solvent solution for a specific period of time (seconds to hours). The rate of immersion into the fibrosis-inhibiting agent/solvent solution can be altered (e.g., 0.001 cm per sec to 50 cm per sec). The device can then be removed from the solution. The rate at which the device can be withdrawn from the solution can be altered (e.g., 0.001 cm per sec to 50 cm per sec). The coated device can be air-dried. The dipping process can be repeated one or more times depending on the specific application. The device can be dried under vacuum to reduce residual solvent levels. In the preferred embodiment, the exposure time of the device to the solvent can be such that there are not significant permanent dimensional changes to the device (other than those associated with the coating itself). The fibrosis-inhibiting agent may also be present on the surface of the device. The amount of surface associated fibrosis-inhibiting agent may be reduced by dipping the coated device into a solvent for the fibrosis-inhibiting agent or by spraying the coated device with a solvent for the fibrosis-inhibiting agent.

In the above description the device can be a device that has not been modified as well as a device that has been further modified by coating with a polymer (e.g., parylene), surface treated by plasma treatment, flame treatment, corona treatment, surface oxidation or reduction, surface etching, mechanical smoothing or roughening, or grafting prior to the coating process.

In another embodiment, a suspension of the fibrosis-inhibiting agent in a polymer solution can be prepared. The suspension can be prepared by choosing a solvent that can dissolve the polymer but not the fibrosis-inhibiting agent or a solvent that can dissolve the polymer and in which the fibrosis-inhibiting agent is above its solubility limit. In similar processes described above, a device can be dipped into the suspension of the fibrosis-inhibiting and polymer solution such that the device is coated with a polymer that has a fibrosis-inhibiting agent suspended within it.

b) Spray Coating

Spray coating is another coating process that can be used. In the spray coating process, a solution or suspension of the fibrosis-inhibiting agent, with or without a polymeric or non-polymeric carrier, is nebulized and directed to the device to be coated by a stream of gas. One can use spray devices such as an air-brush (for example models 2020, 360, 175, 100, 200, 150, 350, 250, 400, 3000, 4000, 5000, 6000 from Badger Air-brush Company, Franklin Park, Ill.), spray painting equipment, TLC reagent sprayers (for example Part # 14545 and 14654, Alltech Associates, Inc. Deerfield, Ill., and ultrasonic spray devices (for example those available from Sono-Tek, Milton, N.Y.). One can also use powder sprayers and electrostatic sprayers.

In one embodiment, the fibrosis-inhibiting agent is dissolved in a solvent for the fibrosis agent and is then sprayed onto the device.

Fibrosis-Inhibiting Agent with an Inert-Solvent

In one embodiment, the solvent is an inert solvent for the device such that the solvent does not dissolve the medical device to any great extent and is not absorbed by the device to any great extent. The device can be held in place or the device can be mounted onto a mandrel or rod that has the ability to move in an X, Y or Z plane or a combination of these planes. Using one of the above described spray devices, the device can be spray coated such that the device is either partially or completely coated with the fibrosis-inhibiting agent/solvent solution. The rate of spraying of the fibrosis-inhibiting agent/solvent solution can be altered (e.g., 0.001 mL per sec to 10 mL per sec) to ensure that a good coating of the fibrosis-inhibiting agent is obtained. The coated device can be air-dried. The spray coating process can be repeated one or more times depending on the specific application. The device can be dried under vacuum to reduce residual solvent levels. This process will result in the fibrosis-inhibiting agent being coated on the surface of the device.

Fibrosis-Inhibiting Agent with a Swelling Solvent

In one embodiment, the solvent is one that will not dissolve the device but will be absorbed by the device. These solvents can thus swell the device to some extent. The device can be spray coated, either partially or completely, in the fibrosis-inhibiting agent/solvent solution. The rate of spraying of the fibrosis-inhibiting agent/solvent solution can be altered (e.g., 0.001 mL per sec to 10 mL per sec) to ensure that a good coating of the fibrosis-inhibiting agent is obtained. The coated device can be air-dried. The spray coating process can be repeated one or more times depending on the specific application. The device can be dried under vacuum to reduce residual solvent levels. This process will result in the fibrosis-inhibiting agent being adsorbed into the medical device. The fibrosis-inhibiting agent may also be present on the surface of the device. The amount of surface associated fibrosis-inhibiting agent may be reduced by dipping the coated device into a solvent for the fibrosis-inhibiting agent or by spraying the coated device with a solvent for the fibrosis-inhibiting agent.

Fibrosis-Inhibiting Agent with a Solvent

In one embodiment, the solvent is one that will be absorbed by the device and that will dissolve the device. The device can be spray coated, either partially or completely, in the fibrosis-inhibiting agent/solvent solution. The rate of spraying of the fibrosis-inhibiting agent/solvent solution can be altered (e.g., 0.001 mL per sec to 10 mL per sec) to ensure that a good coating of the fibrosis-inhibiting agent is obtained. The coated device can be air-dried. The spray coating process can be repeated one or more times depending on the specific application. The device can be dried under vacuum to reduce residual solvent levels. This process will result in the fibrosis-inhibiting agent being adsorbed into the medical device as well as being surface associated. In the preferred embodiment, the exposure time of the device to the solvent can be such that there are not significant permanent dimensional changes to the device. The fibrosis-inhibiting agent may also be present on the surface of the device. The amount of surface associated fibrosis-inhibiting agent may be reduced by dipping the coated device into a solvent for the fibrosis-inhibiting agent or by spraying the coated device with a solvent for the fibrosis-inhibiting agent.

In the above description the device can be a device that has not been modified as well as a device that has been further modified by coating with a polymer (e.g., parylene), surface treated by plasma treatment, flame treatment, corona treatment, surface oxidation or reduction, surface etching, mechanical smoothing or roughening, or grafting prior to the coating process.

In one embodiment, the fibrosis-inhibiting agent and a polymer are dissolved in a solvent, for both the polymer and the anti-fibrosing agent, and are then spray coated onto the device.

Fibrosis-Inhibiting Agent/Polymer with an Inert-Solvent

In one embodiment, the solvent is an inert solvent for the device such that the solvent does not dissolve the medical device to any great extent and is not absorbed by the device to any great extent. The device can be spray coated, either partially or completely, in the fibrosis-inhibiting agent/polymer/solvent solution for a specific period of time. The rate of spraying of the fibrosis-inhibiting agent/solvent solution can be altered (e.g., 0.001 mL per sec to 10 mL per sec) to ensure that a good coating of the fibrosis-inhibiting agent is obtained. The coated device can be air-dried. The spray coating process can be repeated one or more times depending on the specific application. The device can be dried under vacuum to reduce residual solvent levels. This process will result in the fibrosis-inhibiting agent/polymer being coated on the surface of the device.

Fibrosis-Inhibiting Agent/Polymer with a Swelling Solvent

In one embodiment, the solvent is one that will not dissolve the device but will be absorbed by the device. These solvents can thus swell the device to some extent. The device can be spray coated, either partially or completely, in the fibrosis-inhibiting agent/polymer/solvent solution. The rate of spraying of the fibrosis-inhibiting agent/solvent solution can be altered (e.g., 0.001 mL per sec to 10 mL per sec) to ensure that a good coating of the fibrosis-inhibiting agent is obtained. The coated device can be air-dried. The spray coating process can be repeated one or more times depending on the specific application. The device can be dried under vacuum to reduce residual solvent levels. This process will result in the fibrosis-inhibiting agent/polymer being coated onto the surface of the device as well as the potential for the fibrosis-inhibiting agent being adsorbed into the medical device. The fibrosis-inhibiting agent may also be present on the surface of the device. The amount of surface associated fibrosis-inhibiting agent may be reduced by dipping the coated device into a solvent for the fibrosis-inhibiting agent or by spraying the coated device with a solvent for the fibrosis-inhibiting agent.

Fibrosis-Inhibiting Agent/Polymer with a Solvent

In one embodiment, the solvent is one that will be absorbed by the device and that will dissolve the device. The device can be spray coated, either partially or completely, in the fibrosis-inhibiting agent/solvent solution. The rate of spraying of the fibrosis-inhibiting agent/solvent solution can be altered (e.g., 0.001 mL per sec to 10 mL per sec) to ensure that a good coating of the fibrosis-inhibiting agent is obtained. The coated device can be air-dried. The spray coating process can be repeated one or more times depending on the specific application. The device can be dried under vacuum to reduce residual solvent levels. In the preferred embodiment, the exposure time of the device to the solvent can be such that there are not significant permanent dimensional changes to the device (other than those associated with the coating itself). The fibrosis-inhibiting agent may also be present on the surface of the device. The amount of surface associated fibrosis-inhibiting agent may be reduced by dipping the coated device into a solvent for the fibrosis-inhibiting agent or by spraying the coated device with a solvent for the fibrosis-inhibiting agent.

In the above description the device can be a device that has not been modified as well as a device that has been further modified by coating with a polymer (e.g., parylene), surface treated by plasma treatment, flame treatment, corona treatment, surface oxidation or reduction, surface etching, mechanical smoothing or roughening, or grafting prior to the coating process.

In another embodiment, a suspension of the fibrosis-inhibiting agent in a polymer solution can be prepared. The suspension can be prepared by choosing a solvent that can dissolve the polymer but not the fibrosis-inhibiting agent or a solvent that can dissolve the polymer and in which the fibrosis-inhibiting agent is above its solubility limit. In similar processes described above, the suspension of the fibrosis-inhibiting and polymer solution can be sprayed onto the device such that the device is coated with a polymer that has a fibrosis-inhibiting agent suspended within it.

D. Methods for Utilizing Medical Implants

There are numerous medical devices where the occurrence of a fibrotic reaction will adversely affect the functioning of the device or the biological problem for which the device was implanted or used. Representative examples of implants or devices that can be coated with or otherwise constructed to contain and/or release the therapeutic agents provided herein include cardiovascular devices (e.g., implantable venous catheters, venous ports, tunneled venous catheters, chronic infusion lines or ports, including hepatic artery infusion catheters, pacemakers and pacemaker leads, implantable defibrillators; neurologic/neurosurgical devices (e.g., ventricular peritoneal shunts, ventricular atrial shunts, dural patches and implants to prevent epidural fibrosis post-laminectomy, devices for continuous subarachnoid infusions); gastrointestinal devices (e.g., chronic indwelling catheters, feeding tubes, portosystemic shunts, shunts for ascites, peritoneal implants for drug delivery, peritoneal dialysis catheters, and suspensions or solid implants to prevent surgical adhesions); genitourinary devices (e.g., uterine implants, including intrauterine devices (IUDs) and devices to prevent endometrial hyperplasia, fallopian tubal implants, including reversible sterilization devices, fallopian tubal stents, ureteric stents, chronic indwelling catheters, bladder augmentations, or wraps or splints for vasovasostomy, central venous catheters, urinary catheters; prosthetic heart valves, vascular grafts, ophthalmologic implants (e.g., multino implants and other implants for neovascular glaucoma, drug eluting contact lenses for pterygiums, splints for failed dacrocystalrhinostomy, drug eluting contact lenses for corneal neovascularity, implants for diabetic retinopathy, drug eluting contact lenses for high risk corneal transplants); otolaryngology devices (e.g., ossicular implants, Eustachian tube splints or stents for glue ear or chronic otitis as an alternative to transtempanic drains); catheter cuffs and orthopedic implants (e.g., cemented orthopedic prostheses).

Other examples of implants include drainage tubes, biliary T-tubes, clips, sutures, braids, meshes (e.g., hernia meshes, tissue support meshes), barriers (for the prevention of adhesions), anastomotic devices, anastomotic connectors, ventrical assist devices (e.g., LVAD's), artificial hearts, artificial joints, conduits, irrigation fluids, packing agents, stents, staples, inferior vena cava filters, pumps (e.g., for the delivery of therapeutics), hemostatic implants (e.g., sponges), tissue fillers, surgical adhesion barriers (e.g., INTERCEED, degradable polyester films (e.g., PLLA/PDLLA), CMC/PEO association complexes (e.g., OXIPLEX from Fziomed), hyaluronic acid/CMC films (e.g., SEPRAFILM from Genzyme Corporation), bone grafts, skin grafts, tissue sealants, intrauterine devices (IUD), ligatures, titanium implants (particularly for use in dental applications), chest tubes, nasogastric tubes, percutaneous feeding tubes, colostomy devices, bone wax, and Penrose drains, hair plugs, ear rings, nose rings, and other piercing-associated implants, as well as anaesthetic solutions.

The coating of fibrosis-inhibiting agent(s) onto or incorporation of a fibrosis-inhibiting agent(s) into medical devices provides a solution to the clinical problems that can be encountered with these devices. Alternatively, or additional, compositions that comprise anti-scarring agents can be infiltrated in to the space or onto tissue surrounding the area where medical devices are implanted either before, during or after implantation of the devices.

Described below are examples of medical devices whose functioning can be improved by the use of a fibrosis-inhibiting agent as well as methods for incorporating fibrosis-inhibiting agents into or onto these devices and methods for using such devices.

Intravascular Devices

The present invention provides for the combination of an anti-scarring agent and an intravascular device. “Intravascular devices” refers to devices that are implanted at least partially within the vasculature (e.g., blood vessels). Examples of intravascular devices that can be used to deliver anti-scarring agents to the desired location include, e.g., catheters, balloon catheters, balloons, stents, covered stents, stent grafts, anastomotic connectors, and guidewires.

In one aspect, the present invention provides for the combination of an anti-scarring agent or a composition comprising an anti-scarring agent and an intravascular stent.

“Stent” refers to devices comprising a cylindrical tube (composed of a metal, textile, non-degradable or degradable polymer, and/or other suitable material (such as biological tissue) which maintains the flow of blood from one portion of a blood vessel to another. In one aspect, a stent is an endovascular scaffolding which maintains the lumen of a body passageway (e.g., an artery) and allows bloodflow. Representative examples of stents that can benefit from being coated with or having incorporated therein, a fibrosis-inhibiting agent include vascular stents, such as coronary stents, peripheral stents, and covered stents.

Stents that can be used in the present invention include metallic stents, polymeric stents, biodegradable stents and covered stents. Stents may be self-expandable or balloon-expandable, composed of a variety of metal compounds and/or polymeric materials, fabricated in innumerable designs, used in coronary or peripheral vessels, composed of degradable and/or nondegradable components, fully or partially covered with vascular graft materials (so called “covered stents”) or “sleeves”, and can be bare metal or drug-eluting.

Stents may be comprise a metal or metal alloy such as stainless steel, spring tempered stainless steel, stainless steel alloys, gold, platinum, super elastic alloys, cobalt-chromium alloys and other cobalt-containing alloys (including ELGILOY (Combined Metals of Chicago, Grove Village, Ill.), PHYNOX (Alloy Wire International, United Kingdom) and CONICHROME (Carpenter Technology Corporation, Wyomissing, Pa.)), titanium-containing alloys, platinum-tungsten alloys, nickel-containing alloys, nickel-titanium alloys (including nitinol), malleable metals (including tantalum); a composite material or a clad composite material and/or other functionally equivalent materials; and/or a polymeric (non-biodegradable or biodegradable) material. Representative examples of polymers that may be included in the stent construction include polyethylene, polypropylene, polyurethanes, polyesters, such as polyethylene terephthalate (e.g., DACRON or MYLAR (E. I. DuPont De Nemours and Company, Wilmington, Del.)), polyamides, polyaramids (e.g., KEVLAR from E.I. DuPont De Nemours and Company), polyfluorocarbons such as poly(tetrafluoroethylene with and without copolymerized hexafluoropropylene) (available, e.g., under the trade name TEFLON (E. I. DuPont De Nemours and Company), silk, as well as the mixtures, blends and copolymers of these polymers. Stents also may be made with engineering plastics, such as thermotropic liquid crystal polymers (LCP), such as those formed from p,p′-dihydroxy-polynuclear-aromatics or dicarboxy-polynuclear-aromatics.

Further types of stents that can be used with the described therapeutic agents are described, e.g., in PCT Publication No. WO 01/01957 and U.S. Pat. Nos. 6,165,210; 6,099,561; 6,071,305; 6,063,101; 5,997,468; 5,980,551; 5,980,566; 5,972,027; 5,968,092; 5,951,586; 5,893,840; 5,891,108; 5,851,231; 5,843,172; 5,837,008; 5,766,237; 5,769,883; 5,735,811; 5,700,286; 5,683,448; 5,679,400; 5,665,115; 5,649,977; 5,637,113; 5,591,227; 5,551,954; 5,545,208; 5,500,013; 5,464,450; 5,419,760; 5,411,550; 5,342,348; 5,286,254; and 5,163,952. Removable drug-eluting stents are described, e.g., in Lambert, T. (1993) J. Am. Coll. Cardiol.: 21: 483A. Moreover, the stent may be adapted to release the desired agent at only the distal ends, or along the entire body of the stent.

Balloon over stent devices, such as are described in Wilensky, R. L. (1993) J. Am. Coll. Cardiol.: 21: 185A, also are suitable for local delivery of a fibrosing agent to a treatment site.

In addition to using the more traditional stents, stents that are specifically designed for drug delivery can be used. Examples of these specialized drug delivery stents as well as traditional stents include those from Conor Medsystems (Palo Alto, Calif.) (e.g., U.S. Patent. Nos. 6,527,799; 6,293,967; 6,290,673; 6,241,762; U.S. Patent Application Publication Nos. 2003/0199970 and 2003/0167085; and PCT Publication No. WO 03/015664).

Examples of intravascular stents, which may be combined with one or more therapeutic agents according to the present invention, include commercially available products. The stent may be self-expanding or balloon expandable (e.g., STRECKER stent by Medi-Tech/Boston Scientific Corporation), or implanted by a change in temperature (e.g., nitinol stent). Self-expanding stents that can be used include the coronary WALLSTENT and the SCIMED RADIUS stent from Boston Scientific Corporation (Natick, Mass.) and the GIANTURCO stents from Cook Group, Inc. (Bloomington, Ind.). Examples of balloon expandable stents that can be used include the CROSSFLEX stent, BX-VELOCITY stent and the PALMAZ-SCHATZ crown and spiral stents from Cordis Corporation (Miami Lakes, Fla.), the V-FLEX PLUS stent by Cook Group, Inc., the NIR, EXPRESS and LIBRERTE stents from Boston Scientific Corporation, the ACS MULTILINK, MULTILINK PENTA, SPIRIT, and CHAMPION stents from Guidant Corporation, and the Coronary Stent S670 and S7 by Medtronic, Inc. (Minneapolis, Minn.).

Other examples of stents that can be combined with a fibrosing agent in accordance with the invention include those from Boston Scientific Corporation, (e.g., the drug-eluting TAXUS EXPRESS² Paclitaxel-Eluting Coronary Stent System; over the wire stent stents such as the Express² Coronary Stent System and NIR Elite OTW Stent System; rapid exchange stents such as the EXPRESS² Coronary Stent System and the NIR ELITE MONORAIL Stent System; and self-expanding stents such as the MAGIC WALLSTENT Stent System and RADIUS Self Expanding Stent); Medtronic, Inc. (Minneapolis, Minn.) (e.g., DRIVER ABT578-eluting stent, DRIVER ZIPPER MX Multi-Exchange Coronary Stent System and the DRIVER Over-the-Wire Coronary Stent System; the S7 ZIPPER MX Multi-Exchange Coronary Stent System; S7, S670, S660, and BESTENT2 with Discrete Technology Over-the-Wire Coronary Stent System); Guidant Corporation (e.g., cobalt chromium stents such as the MULTI-LINK VISION Coronary Stent System; MULTI-LINK ZETA Coronary Stent System; MULTI-LINK PIXEL Coronary Stent System; MULTI-LINK ULTRA Coronary Stent System; and the MULTI-LINK FRONTIER); Johnson & Johnson/Cordis Corporation (e.g., CYPHER sirolimus-eluting Stent; PALMAZ-SCHATZ Balloon Expandable Stent; and S.M.A.R.T. Stents); Abbott Vascular (Redwood City, Calif.) (e.g., MATRIX LO Stent; TRIMAXX Stent; and DEXAMET stent); Conor Medsystems (Menlo Park, Calif.) (e.g., MEDSTENT and COSTAR stent); AMG GmbH (Germany) (e.g., PICO Elite stent); Biosensors International (Singapore) (e.g., MATRIX stent, CHAMPION Stent (formerly the S-STENT), and CHALLENGE Stent); Biotronik (Switzerland) (e.g., MAGIC AMS stent); Clearstream Technologies (Ireland) (e.g., CLEARFLEX stent); Cook Inc. (Bloomington, Ind.) (e.g., V-FLEX PLUS stent, ZILVER PTX self-expanding vascular stent coating, LOGIX PTX stent (in development); Devax (e.g., AXXESS stent) (Irvine, Calif.); DISA Vascular (Pty) Ltd (South Africa) (e.g., CHROMOFLEX Stent, S-FLEX Stent, S-FLEX Micro Stent, and TAXOCHROME DES); Intek Technology (Baar, Switzerland) (e.g., APOLLO stent); Orbus Medical Technologies (Hoevelaken, The Netherlands) (e.g., GENOUS); Sorin Biomedica (Saluggia, Italy) (e.g., JANUS and CARBOSTENT); and stents from Bard/Angiomed GmbH Medizintechnik KG (Murray Hill, N.J.), and Blue Medical Supply & Equipment (Marietta, Ga.), Aachen Resonance GmbH (Germany); Eucatech AG (Germany), Eurocor GmbH (Bonn, Gemany), Prot, Goodman, Terumo (Japan), Translumina GmbH (Germany), MIV Therapeutics (Canada), Occam International B.V. (Eindhoven, The Netherlands), Sahajanand Medical Technologies PVT LTD. (India); AVI Biopharma/Medtronic/Interventional Technologies (Portland, Oreg.) (e.g., RESTEN NG-coated stent); and Jomed (e.g., FLEXMASTER drug-eluting stent) (Sweden).

Generally, stents are inserted in a similar fashion regardless of the site or the disease being treated. Briefly, a preinsertion examination, usually a diagnostic imaging procedure, endoscopy, or direct visualization at the time of surgery, is generally first performed in order to determine the appropriate positioning for stent insertion. A guidewire is then advanced through the lesion or proposed site of insertion, and over this is passed a delivery catheter which allows a stent in its collapsed form to be inserted. Intravascular stents may be inserted into an artery such as the femoral artery in the groin and advanced through the circulation under radiological guidance until they reach the anatomical location of the plaque in the coronary or peripheral circulation. Typically, stents are capable of being compressed, so that they can be inserted through tiny cavities via small catheters, and then expanded to a larger diameter once they are at the desired location. The delivery catheter then is removed, leaving the stent standing on its own as a scaffold. Once expanded, the stent physically forces the walls of the passageway apart and holds them open. A post insertion examination, usually an x-ray, is often utilized to confirm appropriate positioning.

Stents are typically maneuvered into place under, radiologic or direct visual control, taking particular care to place the stent precisely within the vessel being treated. In certain aspects, the stent can further include a radio-opaque, echogenic material, or MRI responsive material (e.g., MRI contrast agent) to aid in visualization of the device under ultrasound, fluoroscopy and/or magnetic resonance imaging. The radio-opaque or MRI visible material may be in the form of one or more markers (e.g., bands of material that are disposed on either end of the stent) that may be used to orient and guide the device during the implantation procedure.

In another aspect, the present invention provides for the combination of an anti-scarring agent or a composition comprising an anti-scarring agent and an intravascular catheter.

“Intravascular Catheter” refers to any intravascular catheter containing one or more lumens suitable for the delivery of aqueous, microparticulate, fluid, or gel formulations into the bloodstream or into the vascular wall. These formulations may contain a biologically active agent (e.g., an anti-scarring agent). Numerous intravascular catheters have been described for direct, site-specific drug delivery (e.g., microinjector catheters, catheters placed within or immediately adjacent to the target tissue), regional drug delivery (i.e., catheters placed in an artery that supplies the target organ or tissue), or systemic drug delivery (i.e., intra-arterial and intravenous catheters placed in the peripheral circulation). For example, catheters and balloon catheters can deliver anti-fibrosing agents from an end orifice, through one or more side ports, through a microporous outer structure, or through direct injection into the desired tissue or vascular location.

A variety of catheters are available for regional or localized arterial drug-delivery. Intravascular balloon and non-balloon catheters for delivering drugs are described, for example, in U.S. Pat. Nos. 5,180,366; 5,171,217; 5,049,132; 5,021,044; 6,592,568; 5,304,121; 5,295,962; 5,286,254; 5,254,089; 5,112,305; PCT Publication Nos WO 93/08866, WO 92/11890, and WO 92/11895; and Riessen et al. (1994) JACC 23: 1234-1244, Kandarpa K. (2000) J. Vasc. Interv. Radio. 11 (suppl.): 419-423, and Yang, X. (2003) Imaging of Vascular Gene Therapy 228(1): 36-49.

Representative examples of drug delivery catheters include balloon catheters, such as the CHANNEL and TRANSPORT balloon catheters from Boston Scientific Corporation (Natick, Mass.) and Stack Perfusion Coronary Dilitation catheters from Advanced Cardiovascular Systems, Inc. (Santa Clara, Calif.). Other examples of drug delivery catheters include infusion catheters, such as the CRESCENDO coronary infusion catheter available from Cordis Corporation (Miami Lakes, Fla.), the Cragg-McNamara Valved Infusion Catheter available from Microtherapeutics, Inc. (San Clemente, Calif.), the DISPATCH catheter from Boston Scientific Corporation, the GALILEO Centering Catheter from Guidant Corporation (Houston, Tex.), and infusion sleeve catheters, such as the INFUSASLEEVE catheter from LocalMed, Inc. (Sunnyvale, Calif.). Infusion sleeve catheters are described in, e.g., U.S. Pat. Nos. 5,318,531; 5,336,178; 5,279,565; 5,364,356; 5,772,629; 5,810,767; and 5,941,868. Catheters that mechanically or electrically enhance drug delivery include, for example, pressure driven catheters (e.g., needle injection catheters having injector ports, such as the INFILTRATOR catheter available from InterVentional Technologies, Inc. (San Diego, Calif.)) (see, e.g., U.S. Pat. No. 5,354,279) and ultrasonically assisted (phonophoresis) and iontophoresis catheters (see, e.g., Singh, J., et al. (1989) Drug Des. Deliv.: 4: 1-12 and U.S. Pat. Nos. 5,362,309; 5,318,014; 5,315,998; 5,304,120; 5,282,785; and 5,267,985).

In one aspect, the present invention provides for the combination of an anti-scarring agent or a composition comprising an anti-scarring agent and a drug delivery balloon.

“Drug-Delivery Balloon” refers to an intra-arterial balloon (typically based upon percutaneous angioplasty balloons) suitable for insertion into a peripheral artery (typically the femoral artery) and manipulated via a catheter to the treatment site (either in the coronary or peripheral circulation). Numerous drug delivery balloons have been developed for local delivery of therapeutic agents to the arterial wall such as “sweaty balloons,” “channel balloons,” “microinjector balloons,” “double balloons,” “spiral balloons” and other specialized drug-delivery balloons. Other examples of balloons include BHP balloons and Transurethral and Radiofrequency Needle Ablation (TUNA or RFNA)) balloons for prostate applications.

In addition, numerous drug delivery balloons have been developed for local delivery of therapeutic agents to the arterial wall. Representative examples of drug delivery balloons include porous (WOLINSKY) balloons, available from Advanced Polymers (Salem, N.H.), described in, e.g., U.S. Pat. No. 5,087,244. Microporous and macroporous balloons (i.e., “sweaty balloons”) for use in infusion catheters are described in, e.g., Lambert, C. R. et al. (1992) Circ. Res. 71: 27-33. Other types of specialized drug delivery balloons include hydrogel coated balloons (e.g., ULTRATHIN GLIDES from Boston Scientific Corporation) (see, e.g., Fram, D. B. et al. (1992) Circulation: 86 Suppl. I: 1-380), “channel balloons” (see, e.g., U.S. Pat. Nos. 5,860,954; 5,843,033; and 5,254,089, and Hong, M. K., et al. (1992) Circulation: 86 Suppl. I: 1-380), “microinjector balloons” (see, e.g., U.S. Pat. Nos. 5,681,281 and 5,746,716), “double balloons,” described in, e.g., U.S. Pat. No. 6,544,221, and double-layer channeled perfusion balloons (such as the REMEDY balloon from Boston Scientific Corportion), and “spiral balloons” (see, e.g., U.S. Pat. Nos. 6,527,739 and 6,605,056). Drug delivery catheters that include helical (i.e., spiral) balloons are described in, e.g., U.S. Pat. Nos. 6,190,356; 5,279,546; 5236424, 5,226,888; 5,181,911; 4,824,436; and 4,636,195.

The balloon catheter systems that can be used include systems in which the balloon can be inflated at the desired location the desired fibrosis-inducing agents can be delivered through holes that are located in the balloon wall. Other balloon catheters that can be used include systems that have a plurality of holes that are located between two balloons. The system can be guided into the desired location such that the inflatable balloon components are located on either side of the specific site that is to be treated. The balloons can then be inflated to isolate the treatment area. The compositions containing the fibrosing agent are then injected into the isolated area through the plurality of holes between the two balloons. Representative examples of these types of drug delivery balloons are described in U.S. Pat. Nos. 5,087,244, 6,623,452, 5,397,307, 4,636,195 and 4,994,033.

The compositions of the invention can be delivered using a catheter that has the ability to enhance uptake or efficacy of the compositions of the invention. The stimulus for enhanced uptake can include the use of heat, the use of cooling, the use of electrical fields or the use of radiation (e.g., ultraviolet light, visible light, infrared, microwaves, ultrasound or X-rays). Further Representative examples of catheter systems that can be used are described in U.S. Patent. Nos. and 2002/0068869; and PCT Publication Nos. WO 01/15771; WO 94/05361; WO 96/04955 and WO 96/22111.

In another aspect of the invention, the compositions of the inventions can be delivered into the treatment site and/or into the tissue surrounding the treatment site by using catheter systems that have one or more injectors that can penetrate the surrounding tissue. Following insertion into the appropriate vessel, the catheter can be maneuvered into the desired position such that the injectors are aligned with or adjacent to the tissue. The injector(s) are inserted into the desired location, for example by direct insertion into the tissue, by inflating the balloon or mechanical rotation of the injector, and the composition of the invention is injected into the desired location. Representative examples of catheters that can be used for this application are described in and U.S. Patent Application Publication No. 2002/0082594 and U.S. Pat. Nos. 6,443,949; 6,488,659; 6,569,144; 5,609,151; 5,385,148; 5,551,427; 5,746,716; 5,681,281; and 5,713,863.

In another aspect of the invention, the catheter may be adapted to deliver a thermoreversible polymer composition. For the site-specific delivery of these materials, a catheter delivery system has the ability to either heat the composition to above body temperature or to cool the composition to below body temperature such that the composition remains in a fluent state within the catheter delivery system. The catheter delivery system can be guided to the desired location and the composition of the invention can be delivered to the surface of the surrounding tissue or can be injected directly into the surrounding tissue. A representative example of a catheter delivery system for direct injection of a thermoreversible material is described in U.S. Pat. No. 6,488,659. Representative examples of catheter delivery systems that can deliver the thermoreversible compositions to the surface of the vessel are described in U.S. Pat. Nos. 6,443,941; 6,290,729; 5,947,977; 5,800,538; and 5,749,922.

In another aspect, the present invention provides for the combination of an anti-scarring agent or a composition comprising an anti-scarring agent and an anastomotic connector device.

“Anasomotic connector device” refers to any vascular device that mechanizes the creation of a vascular anastomosis (i.e., artery-to-artery, vein-to-artery, artery-to-vein, artery-to-synthetic graft, synthetic graft-to-artery, vein-to-synthetic graft or synthetic graft-to-vein anastomosis) without the manual suturing that is typically done in the creation of an anastomosis. The term also refers to anastomotic connector devices (described below), designed to produce a facilitated semiautomatic vascular anastomosis without the use of suture and reduce connection time substantially (often to several seconds), where there are numerous types and designs of such devices. The term also refers to devices which facilitate attachment of a vascular graft to an aperture or orifice (e.g., in the side or at the end of a vessel) in a target vessel. Anastomotic connector devices may be anchored to the outside of a blood vessel, and/or into the wall of a blood vessel (e.g., into the adventitial, intramural, or intimal layer of the tissue), and/or a portion of the device may reside within the lumen of the vessel.

Anastomotic connector devices also may be used to create new flow from one structure to another through a channel or diversionary shunt. Accordingly, such devices (also referred to herein as “bypass devices”) typically include at least one tubular structure, wherein a tubular structure defines a lumen. Anastomotic connector devices may include one tubular structure or a plurality of tubular structures through which blood can flow. At least a portion of the tubular structure resides external to a blood vessel (i.e., extravascular) to provide a diversionary passageway. A portion of the device also may reside within the lumen and/or within the tissue of the blood vessel.

Examples of anastomotic connector devices are described in co-pending application entitled, “Anastomotic Connector Devices”, filed May 23, 2003 (U.S. Ser. No. 60/473,185). Representative examples of anastomotic connector devices include, without limitation, vascular clips, vascular sutures, vascular staples, vascular clamps, suturing devices, anastomotic coupling devices (i.e., anastomotic couplers), including couplers that include tubular segments for carrying blood, anastomotic rings, and percutaneous in situ coronary artery bypass (PISCAB and PICVA) devices. Broadly, anastomotic connector devices may be classified into three categories: (1) automated and modified suturing methods and devices, (2) micromechanical devices, and (3) anastomotic coupling devices.

(1) Automated and Modified Suturing Methods and Devices

Automated sutures and modified suturing methods generally facilitate the rapid deployment of multiple sutures, usually in a single step, and eliminate the need for knot tying or the use of aortic side-biting clamps. Suturing devices include those devices that are adapted to be minimally invasive such that anastomoses are formed between vascular conduits and hollow organ structures by applying sutures or other surgical fasteners through device ports or other small openings. With these devices, sutures and other fasteners are applied in a relatively quick and automated manner within bodily areas that have limited access. By using minimally invasive means for establishing anastomoses, there is less blood loss and there is no need to temporarily stop the flow of blood distal to the operating site. For example, the suturing device may be composed of a shaft-supported vascular conduit that is adapted for anastomosis and a collar that is slideable on the shaft configured to hold a plurality of needles and sutures that passes through the vascular conduit. See, e.g., U.S. Pat. No. 6,709,441. The suturing device may be composed of a carrier portion for inserting graft, arm portions that extend to support the graft into position, and a needle assembly adapted to retain and advance coil fasteners into engagement with the vessel wall and the graft flange to complete the anastomosis. See, e.g., U.S. Pat. No. 6,709,442. The suturing device may include two oblong interlinked members that include a split bush adapted for suturing (e.g., U.S. Pat. No. 4,350,160).

One representative example of a suturing device is the HEARTFLOW device, made by Perclose-Abbott Labs, Redwood City, Calif. (see generally, U.S. Pat. Nos. 6,358,258, 6,355,050, 6,190,396, and 6,036,699, and PCT Publication No. WO 01/19257).

The nitinol U-CLIP suture clip device by Coalescent Surgical (Sunnyvale, Calif.) consists of a self-closing nitinol wire loop attached to a flexible member and a needle with a quick release mechanism. This device facilitates the construction of anastomosis by simplifying suture management and eliminating knot tying (see generally, U.S. Pat. Nos. 6,074,401 and 6,149,658, and PCT Publication Nos. WO 99/62406, WO 99/62409, WO 00/59380, WO 01/17441).

The ENCLOSE Anastomotic Assist Device (Novare Surgical Systems, Cupertino, Calif.) allows a surgeon to create a sutured anastomosis using standard suturing techniques but without the use of a partial occluding side-biting aortic clamp, avoiding aortic wall distortion (see U.S. Pat. Nos. 6,312,445 and 6,165,186).

In one aspect, automated and modified suturing methods and devices can deliver a surgical fastener (e.g., a suture or suture clip) that comprises an anti-scarring agent. In another aspect, automated and modified suturing methods and devices can deliver a vascular graft that comprises an anti-scarring agent to complete an anastomosis.

(2) Micromechanical Devices

Micromechanical devices are used to create an anastomosis and/or secure a graft vessel to the site of an anastomosis. Representative examples of micromechanical devices include staples (either penetrating or non-penetrating) and clips.

Anastomotic staple and clip devices may take a variety of forms and may be made from different types of materials. For example, staples and clips may be formed of a metal or metal alloy, such as titanium, nickel-titanium alloy, or stainless steel, or a polymeric material, such as silicone, poly(urethane), rubber, or a thermoplastic elastomer.

The polymeric material may be an absorbable or biodegradable material designed to dissolve after completion of the anastomosis. Biodegradable polymers include, for example, homopolymers and copolymers that comprise one or more of the monomers selected from lactide, lactic acid, glycolide, glycolic acid, ε-caprolactone, gamma-caprolactone, hydroxyvaleric acid, hydroxybutyric acid, beta-butyrolactone, gamma-butyrolactone, gamma-valerolactone, ?-decanolactone, d-decanolactone, trimethylene carbonate, 1,4-dioxane-2-one or 1,5-dioxepan-2one.

A variety of devices for guiding staples and clips into position also have been described.

One manufacturer of non-penetrating staples for use in the creation of anastomosis is United States Surgical Corp. (Norwalk, Conn.). The VCS system (Autosuture) is an automatic stapling device that applies non-penetrating, titanium vascular clips which are usually used in an interrupted fashion to evert tissue edges with high compressive forces. (See, e.g., U.S. Pat. Nos. 6,440,146, 6,391,039, 6,024,748, 5,833,698, 5,799,857, 5,779,718, 5,725,538, 5,725,537, 5,720,756, 5,360,154, 5,193,731, and 5,005,749 for the description of anastomotic connector devices made by U.S. Surgical).

An anastomotic clip may be composed of a shape memory material, such as nitinol, which is self-closing between an open U-shaped configuration and a closed configuration. See, e.g., U.S. Pat. No. 6,641,593. The anastomotic clip may be composed of a wire having a shape memory that defines a closed configuration which may be substantially spiral-shaped and having a needle that may be releasably attached to the clip. See, e.g., U.S. Pat. No. 6,551,332. Other anastomotic clips are described in, e.g., U.S. Pat. Nos. 6,461,365; and 6,514,265.

Automatic stapling devices are also made by Bypass/Ethicon, Inc. (Somerville, N.J.) and are described in, e.g., U.S. Pat. Nos. 6,193,129; 5,632,433; 5,609,285; 5,533,661; 5,439,156; 5,350,104; 5,333,773; 5,312,024; 5,292,053; 5,285,945; 5,275,322; 5,271,544; 5,271,543 and 5,205,459 and WO 03/02016. Resorbable surgical staples that include a polymer blend that is rich in glycolide (i.e., 65 to 85 weight % polymerized glycolide) are described in, e.g., U.S. Pat. Nos. 4,741,337 and 4,889,119. Surgical staples made from a blend of lactide/glycolide-copolymer and poly(p-dioxanone) are described in U.S. Pat. No. 4,646,741. Other types of stapling devices are described in, e.g., U.S. Pat. Nos. 5,234,447; 5,904,697 and 6,565,582; and U.S. Publication No. 2002/0185517A1.

In another aspect, the micromechanical device may be an anastomotic clip. For example, an anastomotic clip may be composed of a shape memory material, such as nitinol, which is self-closing between an open U-shaped configuration and a closed configuration. See, e.g., U.S. Pat. No. 6,641,593. The anastomotic clip may be composed of a wire having a shape memory that defines a closed configuration which may be substantially spiral-shaped and having a needle that may be releasably attached to the clip. See, e.g., U.S. Pat. No. 6,551,332. Other anastomotic clips are described in, e.g., U.S. Pat. Nos. 6,461,365; 6,187,019; and 6,514,265.

In one aspect, the present invention provides for the combination of a micromechanical anastomotic device (e.g., a staple or a clip) and an anti-scarring agent.

(3) Anastomotic Coupling Devices

Anastomotic coupling devices may be used to connect a first blood vessel to a second vessel, either with or without a graft vessel, for completion of an anastomosis. In one aspect, anastomotic coupling devices facilitate automated attachment of a graft or vessel to an aperture or orifice (e.g., in the side or at the end of a vessel) in a target vessel without the use of sutures or staples. In another aspect, the anastomotic coupling device comprises a tubular structure defining a lumen through which blood may flow (described below).

Anastomotic coupling devices that facilitate automated attachment of a graft or vessel to an aperture or orifice in a target vessel may take a variety of forms and may be made from a variety of materials. Typically, such devices are made of a biocompatible material, such as a polymer or a metal or metal alloy. For example, the device may be formed from a synthetic material, such as a fluoropolymer, such as expanded poly(tetrafluoroethylene) (ePTFE) (ePTFE) sold under the trade name GORE-TEX available from W.L. Gore & Associates, Inc. or fluorinated ethylene propylene (FEP), a polyurethane, polyethylene, polyamide (nylon), silicone, polypropylene, polysulfone, or a polyester.

Anastomotic coupling devices may include an absorbable or biodegradable material designed to dissolve after completion of the anastomosis. Biodegradable polymers include, for example, homopolymers and copolymers that comprise one or more of the monomers selected from lactide, lactic acid, glycolide, glycolic acid, ε-caprolactone, gamma-caprolactone, hydroxyvaleric acid, hydroxybutyric acid, beta-butyrolactone, gamma-butyrolactone, gamma-valerolactone, ?-decanolactone, d-decanolactone, trimethylene carbonate, 1,4-dioxane-2-one or 1,5-dioxepan-2one.

The device may include a metal or metal alloy (e.g., nitinol, stainless steel, titanium, iron, nickel, nickel-titanium, cobalt, platinum, tungsten, tantalum, silver, gold, molybdenum, chromium, and chrome), or a combination of a metal and a polymer.

The device may be anchored to the outside of a vessel, within the tissue that surrounds the lumen of a blood vessel, and/or a portion of the device may reside within the lumen of the vessel.

In one aspect, the anastomotic coupler may be an artificially formed aperture connector that is placed in the side wall of the target vessel so that the tubular graft conduit may be extended from the target vessel. The connector may include a plurality of tissue-piercing members and retention fingers disposed in a concentric annular array which may be passed through the side wall of the tubular graft conduit for securing and retaining the graft to the connector in a fluid-tight configuration. See, e.g., U.S. Pat. Nos. 6,702,829 and 6,699,256.

In another aspect, the anastomotic coupler may be in the form of a frame. For example, the frame may be configured to be deformable and scissor-shaped such that spreading members are moveable to secure a graft vessel upon insertion into a target vessel. See, e.g., U.S. Pat. No. 6,179,849.

In another aspect, the anastomotic coupler may be a ring-like device that is used as an anastomotic interface between a lumen of a graft and an opening in a lumen of a target vessel. For example, the anastomotic ring may be composed of stainless steel alloy, titanium alloy, or cobalt alloy and have a flange with an expandable diameter. See, e.g., U.S. Pat. No. 6,699,257. Anastomosis rings are also described in, e.g., U.S. Pat. No. 6,248,117.

In another aspect, the anastomotic coupler is resorbable. Resorbable anastomotic coupling devices may include, for example, a polymeric blend that is rich in glycolide (i.e., 65 to 85 weight % polymerized glycolide) (see, e.g., U.S. Pat. Nos. 4,741,337 and 4,889,119) or a blend of lactide/glycolide-copolymer and poly(p-dioxanone) (see, e.g., U.S. Pat. No. 4,646,741).

In another aspect, the anastomotic coupler includes a bioabsorbable, elastomeric material. Representative examples of elastomeric materials for use in resorbable devices are described in, e.g., U.S. Pat. No. 5,468,253.

In another aspect, the anastomotic coupler may be used to connect a first blood vessel to a second vessel, either with or without a graft vessel. For example, the anastomotic coupler may be a device that serves to interconnect two vessels in a side-to-side anastomosis, such as when grafting two juxtaposed cardiac vessels. The anastomotic coupler may be configured as two partially opened cylindrical segments that are interconnected along the periphery by a flow opening whereby the device may be inserted in a minimally-invasive manner which then conforms to provide pressure against the interior wall when in the original configuration such that leakage is prevented. See, e.g., U.S. Pat. Nos. 6,464,709; 6,458,140 and 6,251,116 and U.S. Application Publication No. 2003/0100920A1.

In another aspect, the anastomotic coupler may also be incorporated in the design of a vascular graft to eliminate the step of attaching the interface prior to deployment. For example, the anastomotic coupler may have a leading and rear petal for dilating the vessel opening during advancement, and a base which is configured for attachment to a graft while forming a seal with the opening of the vessel. See, e.g., U.S. Pat. No. 6,702,828.

In another aspect, the anastomotic coupler may be in the form of a frame. For example, the anastomotic coupler may be composed of a deformable, scissor-shaped frame with spreading members that is inserted into a target vessel. See, e.g., U.S. Pat. No. 6,179,849.

In another aspect, the anastomotic coupling device may include a graft that incorporates fixation mechanisms (e.g., a collet or a grommet) at its opposite ends and a heating element to create a thermal bond between the graft and a blood vessel (see, e.g., U.S. Pat. Nos. 6,652,544 and 6,293,955).

In another aspect, the anastomotic coupling device includes a compressible, expandable fitting for securing the ends of a bypass graft to two vessels. The fitting may be incorporated in the bypass graft design to eliminate the step of attaching the graft to the fitting prior to deployment (see, e.g., U.S. Pat. No. 6,494,889).

In another aspect, the anastomotic coupling device includes a pair of coupling disc members for joining two vessels in an end-to-end or end-to-side fashion. One of the members includes hook members, while the other member has receptor cavities aligned with the hooks for locking everted tissue of the vessels together (see, e.g., U.S. Pat. No. 4,523,592).

Representative examples of anastomotic connector devices of Bypass/Ethicon, Inc. are described in U.S. Application Publication Nos. U.S. 2002/0082625A1 and 2003/0100910A1 and U.S. Pat. Nos. 6,036,703, 6,036,700, 6,015,416, and 5,346,501.

Other anastomotic coupling devices are those described in e.g., U.S. Pat. Nos. 6,036,702; 6,508,822; 6,599,303; 6,673,084, 5,695,504; 6,569,173; 4,931,057; 5,868,763; 4,624,257; 4,917,090; 4,917,091; 5,697,943; 5,562,690; 5,454,825; 5,447,514; 5,437,684; 5,376,098; 6,652,542; 6,551,334; and 6,726,694 and U.S. Application Publication Nos. 2003/0120293A1 and 2004/0030348A1.

Anastomotic coupling devices may include proximal aortic connectors and distal coronary connectors. For example, aortic anastomotic connectors include devices such as the SYMMETRY Bypass Aortic Connector device made by St. Jude Medical, Inc. (Maple Grove, Minn.), which consists of an aortic cutter or hole punch assembly and a graft delivery system. The aortic hole punch is a cylindrical cutter with a barbed needle that provides an anchor and back pressure for the rotating cutter to core a round hole in the wall of the aorta. The graft delivery system is a radially expandable nitinol device that holds the vein graft with small hooks which pierce through vein graft wall. The graft is fixed to the aorta through use of an inner and outer ring of struts or flanges. This and other anastomotic connector devices by St. Jude are described in U.S. Pat. Nos. 6,309,416, 6,302,905, 6,152,937, and PCT Publication Nos. WO 00/27312 and WO 00/27311.

The CORLINK Automated Anastomotic connector device, which is produced by the CardioVations division of Ethicon, Inc. (Johnson & Johnson, Somerville, N.J.), uses a nitinol metal alloy fastener to connect the grafted vessel to the aorta. It consists of a central cylindrical body made of interconnected elliptical arches and two sets of several pins radiating from each end. The graft is loaded into a CORLINK insertion instrument and deployed to create an anastomosis in one step.

Further examples of anastomotic coupling devices include those made by Cardica (see, U.S. Pat. Nos. 6,719,769; 6,419,681 and 6,537,287), Converge Medical (formerly Advanced Bypass Technologies), Onux Medical (see, e.g., PCT Publication No. WO 01/34037) and Ventrica, Menlo Park, Calif. (VENTRICA Magnetic Vascular Positioner) (see, e.g., U.S. Pat. Nos. 6,719,768; 6,517,558 and 6,352,543).

As described above, an anastomotic coupling device may comprise a tubular structure defining a lumen through which blood may flow. These types of devices (also referred to herein as “bypass devices”) can function as an artificial passageway or conduit for fluid communication between blood vessels and can be used to divert (i.e., shunt) blood from one part of a blood vessel (e.g., an artery) to another part of the same vessel, or to a second vessel (e.g., an artery or a vein) or to multiple vessels (e.g., a vein and an artery). In one aspect of the invention, the anastomotic device is a bypass device.

Bypass devices may be used in a variety of end-to-end and end-to-side anastomotic procedures. The bypass device may be placed into a patient where it is desired to create a pathway between two or more vascular structures, or between two different parts of the same vascular structure. For example, bypass devices may be used to create a passageway which allows blood to flow around a blood vessel, such as an artery (e.g., coronary artery, carotid artery, or artery supplying the lower limb), which has become damaged or completely or partially obstructed. Bypass devices may be used in coronary artery bypass surgery to shunt blood from an artery, such as the aorta, to a portion of a coronary artery downstream from an occlusion in the artery.

Certain types of anastomotic coupling devices are configured to join two abutting vessels. The device can further include a tubular segment to shunt blood to another vessel. These types of connectors are often used for end-to-end anastomosis if a vessel is severed or injured.

Bypass devices include at least one tubular structure having a first end and a second end, which defines a single lumen through which blood can flow, or may include more than one tubular structure, defining multiple lumens through which blood can flow. The tubular structure includes an extravascular portion and may, optionally, include an intravascular portion. The extravascular portion resides external to the adventitial tissue of a blood vessel, whereas the intravascular portion may reside within the vessel lumen or within the intimal, medial, and/or adventitial tissue.

The configuration of the tubular segment may take a variety of forms. For example, the tubular portion may be generally straight, bent or curved (e.g., L-shaped or helical), tapered, branched (e.g., bifurcated or trifurcated), or may include a network of conduits through which blood may flow. Generally, straight or bent devices have a single lumen through which blood may flow, while branched conduits (e.g., generally T-shaped and Y-shaped devices) and conduit networks (described below) have two or more lumens through which blood may flow. A tubular structure may be in the form, for example, of a hollow cylinder and may or may not include a support structure, such as a mesh or porous framework. Depending on the procedure, the device may be biodegradable or non-biodegradable; expandable or rigid; metal and/or polymeric; and/or may include a shape-memory material (e.g., nitinol). In certain embodiments, the device may include a self-expanding stent structure.

Bypass devices typically are made of a biocompatible material. Any of the materials described above for other types of connectors may be used to make a bypass device, such as a synthetic or naturally-derived polymer, or a metal or metal alloy. For example, the device may be formed from a synthetic material, such as a fluoropolymer, such as expanded poly(tetrafluoroethylene) (ePTFE) or fluorinated ethylene propylene (FEP), a polyurethane, polyethylene, polyamide (nylon), silicone, polypropylene, polysulfone, or a polyester and/or a naturally derived material, such as collagen or a polysaccharide. The device may include a metal or metal alloy (e.g., nitinol, stainless steel, titanium, nickel, nickel-titanium, cobalt, platinum, iron, tungsten, tantalum, silver, gold, molybdenum, chromium and chrome), or a combination of a metal and a polymer. Other types of devices include a natural graft material (e.g., autologous vessel, homologous vessel, or xenograft), or a combination of a synthetic and a natural graft material. In another aspect, the bypass device may be formed of an absorbable or biodegradable material designed to dissolve after completion of the anastomosis (e.g., polylactide, polyglycolide, and copolymers of lactide and glycolide). In yet another aspect, demineralized bone may be used to provide a pliable tubular conduit (see, e.g., U.S. Pat. No. 6,290,718).

The tubular structure(s) include a proximal end that may be configured for attachment to a proximal blood vessel and a distal end configured for attachment to a distal blood vessel. As described above, an anastomosis may be described as being either “proximal” or “distal” depending on its location relative to the vascular obstruction. The “proximal” anastomosis may be formed in a proximal blood vessel, and the “distal” anastomosis may be formed in a distal blood vessel, which may the same vessel or a different vessel than the proximal vessel. The terms “distal” and “proximal” may also be used to describe the direction that blood flows through a tubular structure from one vessel into another vessel. For example, blood may flow from a proximal vessel (e.g., the aorta) into a distal vessel, such as a coronary artery to bypass an obstruction in the coronary artery.

The tubular structure may be attached directly to a proximal or distal blood vessel. Alternatively, the bypass device may further include a graft vessel or be configured to receive a graft vessel, which can be connected to the same or a different blood vessel for completion of the anastomosis. Representative examples of graft vessels include, for example, vascular grafts or grafts used in hemodialysis applications (e.g., AV graft, AV shunt, or AV graft).

In one aspect, a tubular anastomotic coupler includes a proximal end that is attached to a proximal vessel and a distal end that is used to attach a bypass graft. The bypass graft can be secured to the distal vessel to complete the anastomosis. The direction of blood flow can be from the proximal blood vessel and into the proximal end of the tubular structure. Blood can exit through the distal end of the tubular structure and into the graft vessel.

In another aspect, the tubular anastomotic coupler includes a proximal end that is attached to a graft vessel, which is secured to the proximal blood vessel, and a distal end that is configured for attachment to a distal blood vessel. The direction of blood flow can be from the proximal vessel into the graft vessel and into the proximal end of the tubular structure. Blood can exit through the distal end of the tubular structure and into the distal vessel.

Anastomotic bypass devices may be anchored to a blood vessel in a variety of ways and may be attached to a blood vessel for the formation of an anastomosis with or without the use of sutures. Bypass devices may be attached to the outside of a blood vessel, and/or a portion of the device may be implanted into a vessel. For example, a portion of the implanted device may reside within the lumen of the vessel (i.e., endoluminally), and/or a portion of the implanted device may reside intravascularly (i.e., within the intimal, intramural, and/or adventitial tissue of the blood vessel). In one aspect, at least one of the tubular structures, or a portion thereof, may be inserted into the end of a vessel or into the side of a blood vessel. The device may be secured directly to the vessel using, for example, a fastener, such as sutures, staples, or clips and/or an adhesive. Bypass devices may include an interface to secure the conduit to a target vessel without the use of sutures. The interface may include means, such as, for example, hooks, barbs, pins, clamps, or a flange or lip for coupling the device to the site of an anastomosis.

Representative examples of anastomotic coupling devices that include at least one tubular portion include, without limitation, devices used for end-to-end anastomosis procedures (e.g., anastomotic stents and anastomotic sleeves) and end-to-side anastomosis procedures (e.g., single-lumen and multi-lumen bypass devices).

In one aspect of the invention, the anastomotic coupling device comprises a single tubular portion that may by used as a shunt to divert blood from a source vessel to a graft vessel (e.g., in an end-to-side anastomosis procedure). In one aspect, an end of the tubular portion may be connected directly or indirectly to a target vessel, as described above. The opposite end of the tubular portion may be attached to a graft vessel, where the graft vessel may be secured to a target vessel to complete the anastomosis.

The tubular portion(s) may be straight or may have a curved or bent shape (e.g., L-shaped or helical) and may be oriented orthogonally or at an angle relative to the vessel to which it is connected. In one aspect, the conduit may be secured into the site by, for example, a fastener, such as staples, clamps, or hooks, or by adhesives, radiofrequency sealing, or by other methods known to those skilled in the art.

In one aspect, the anastomotic coupling device may be, for example, a tubular metal braided graft with suture rings welded at the distal end to provide a means for securing in place to the target vessel. See, e.g., U.S. Pat. No. 6,235,054. Other types of conduits that are secured into the site include, e.g., U.S. Pat. Nos. 4,368,736 and 4,366,819.

In certain types of single-lumen coupling devices, the conduit terminates in a flange that resides within the lumen of the vessel. For example, the conduit may have a tubular body with a connector which has a plurality of extensions and is configured for disposition annularly within the inside of a tubular vessel. See, e.g., U.S. Pat. No. 6,660,015. In other devices, the flange may be attached into or onto the surface of the adventitial tissue of the blood vessel.

Other types of single-lumen bypass devices are described, for example, in U.S. Pat. Nos. 6,241,743; 6,428,550; 6,241,743; 6,428,550; 5,904,697; 5,290,298; 6,007,576; 6,361,559; 6,648,901, 4,931,057 and U.S. Application Publication Nos. 2004/0015180A1, 2003/0065344A1, and 2002/0116018A1.

In one aspect of the invention, the anastomotic coupling device comprises more than one lumen through which blood may travel. Multi-lumen bypass devices may include two or more tubular portions configured to interconnect multiple (two or more) blood vessels. Multi-lumen coupling devices may be used in a variety of anastomosis procedures. For example, such devices may be used in coronary artery bypass graft (CABG) surgery to divert blood from an occluded proximal vessel (e.g., an artery) into one or more target (i.e., distal) vessels (e.g., an artery or vein).

In one aspect, at least one tubular portion may by used as a shunt for diverting blood between a source vessel and a target vessel. In another aspect, the device may be configured as an interface for securing a graft vessel to a target vessel for completion of an anastomosis. Depending on the procedure, the tubular arms may be of equal length and diameter or of unequal length and diameter and may include a tubular portion(s) that is expandable and/or includes a shape-memory material (e.g., nitinol). Furthermore, the tubular portions may be made of the same material or a different material.

In one aspect, one or more ends of a tubular portion may be inserted into the end or into the side of one or more blood vessels. In other embodiments, one or more tubular portions of the device may reside within the lumen of a blood or graft vessel. The device, optionally, may be secured to the blood vessel using a fastener or an adhesive, or another approach known to those skilled in the art.

At least one arm of the multi-lumen connector may be attached to a graft vessel. The graft vessel may be a synthetic graft, such as an ePTFE or polyester graft, or natural graft material (e.g., autologous vessel, homologous vessel, or xenograft), or a combination of a synthetic and a natural graft material. In certain embodiments, a graft vessel may be attached to an end of a tubular portion of the device, and a second graft vessel may be attached to the opposite end of the same tubular portion or to the end of another tubular portion. The graft vessel(s) may be further attached to a target vessel(s) for the completion of the anastomosis.

In one aspect, the device may include three or more tubular arms that extend from a junction site. For example, the multi-lumen device may be generally T-shaped or Y-shaped (i.e., having two or three lumens, respectively). For example, the multi-lumen device may be a T-shaped tubular graft connector having a longitudinal member that extends into the target vessel and a second section that is exterior to the vessel which provides a connection to an alternate tubular structure. See, e.g., U.S. Pat. Nos. 6,152,945 and 5,972,017. Other multi-lumen devices are described in, (see, e.g., U.S. Pat. Nos. 6,152,945; 6,451,033; 5,755,778; 5,922,022; 6,293,965; 6,517,558 and 6,626,914 and U.S. Publication No. 2004/0015180A1).

In another aspect, the device may be a tube for bypassing blood flow directly from a portion of the heart (e.g., left ventricle) to a coronary artery. For example, the device may be a hollow tube that may be partially closable by a one-way valve in response to movement of the cardiac tissue during diastole while permitting blood flow during systole (see, e.g., U.S. Pat. No. 6,641,610). The device may be an elongated rigid shunt body composed of a diversion tube having two apertures in which one may be disposed within the cyocardium of the left ventricle and the other may be disposed within the coronary artery (see, e.g., WO 00/15146 and U.S. Application Publication No. 2003/0055371A1). The device may be a valved, tubular apparatus that is L- or T-shaped which is adapted for insertion into the wall of the heart to provide blood communication from the heart to a coronary vessel (see, e.g., U.S. Pat. No. 6,123,682).

In another aspect, the device may include a network of interconnected tubular conduits. For example, the device may include two tubular portions that may be oriented generally axially or orthogonally relative to each other. See U.S. Pat. Nos. 6,241,761 and 6,241,764. Communication between the two tubular structures may be achieved through a flow channel which facilitates blood to flow between the bores of each tube.

In another aspect, the anastomotic coupling device is a resorbable device that may be configured with two or three termini which provide a vessel interface without the need for sutures and provides a fluid communication through an intersecting lumen, such as a bypass graft or alternate vessel. See, e.g., U.S. Application Publication Nos. 2002/0052572A1 and PCT Publication No. WO 02/24114A2. An anastomotic connector may also be formed of a resorbable tubular structure configured to include snap-connectors or other components for securing it to the tissue as well as hemostasis inducing sealing rings to prevent blood leakage. See, e.g., U.S. Pat. Nos. 6,056,762. The anastomotic connector may be designed with three legs whereby two legs are adapted to be inserted within the continuous blood vessel in a contracted state and then enlarged to form a tight fit and the third leg is adapted for connecting and sealing with a third conduit. See, e.g., U.S. Pat. No. 6,019,788.

An example of a commercially available multi-lumen anastomotic coupling device is the SOLEM graft connector (made by Jomed, Sweden). This device, which is described in more detail in PCT Publication No. WO 01/13820, and U.S. Pat. Nos. 6,179,848, D438618 and D429334, includes a T-shaped connector composed of nitinol and an ePTFE graft for completion of a distal anastomosis.

Another example of an anastomotic connector is the HOLLY GRAFT System (in development) for use in bypass surgery from CABG Medical, Inc. (Minneapolis, Minn.), which is described, e.g., in U.S. Pat. Nos. 6,241,761 and 6,241,764.

In one aspect, the present invention provides for the combination of an anastomotic coupling device and an anti-scarring agent or a composition comprising an anti-scarring device. In one aspect, the anastomotic coupling device may be attached to a blood vessel for the formation of an anastomosis without the use of sutures or staples. In certain aspects, the anastomotic coupling device may comprise a tubular structure defining a lumen through which blood may flow, and an anti-scarring agent. The device may include one, two, three, or more lumens defined by one, two, three, or more tubular structures, depending on the number of vessels to be connected.

Introduction of an anastomotic connector into or onto an intramural, luminal, or adventitial portion of a blood vessel may irritate or damage the endothelial tissue of the blood vessel and/or may alter the natural hemodynamic flow through the vessel. This irritation or damage may stimulate a cascade of biological events resulting in a fibrotic response which can lead to the formation of scar tissue in the vessel. Incorporation of a therapeutic agent in accordance with the invention into or onto a portion of the device that is in direct contact with the blood vessel (e.g., a terminal portion or edge of the device) may inhibit one or more of the scarring processes described above (e.g., smooth muscle cell proliferation, cell migration, inflammation), making the vessel less prone to the formation of intimal hyperplasia and stenosis.

Thus, in one aspect, the therapeutic agent may be associated only with the portion of the device that is in contact with the blood or endothelial tissue. For example, the anti-scarring agent may be incorporated into only an intravascular portion (i.e., that portion that resides within the lumen of the vessel or in the vessel tissue) of the device. The anti-scarring agent may be incorporated onto all or a portion of the intravascular portion of the device. In other embodiments, the coating may reside on all or a portion of an extravascular portion of the device.

The anti-scarring agent or a composition that includes an anti-scarring agent may be coated onto a portion of or onto the entire surface of the device or may be incorporated into a portion of, or into the entire the structure of, the device (e.g., either within voids, reservoirs, or divets in the device or within the material used to construct the device). In other aspects, the agent or a composition comprising the agent is impregnated into or affixed onto the device surface.

As described above, the device may include a tubular portion that is disposed within the lumen of a blood vessel. The entire tubular portion may, for example, be coated with an anti-scarring agent or a composition comprising an anti-scarring. Alternatively, only a portion of the tubular portion may include the anti-scarring agent. For example, only an external (abluminal) surface or only the interior (endoluminal) surface of the tubular portion may be coated. In other embodiments, one or both termini of the tubular portion may be coated. For example, the endoluminal and/or abluminal surface of the tubular section through which blood enters into the device (i.e., proximal end) may be coated with the anti-scarring agent or composition comprising the anti-scarring agent. In another aspect, the endoluminal and/or abluminal surface of the tubular section through which blood exits (i.e., distal end) from the device may be coated with the anti-scarring agent or composition comprising the anti-scarring agent.

In another embodiment, the anti-scarring agent or composition comprising the anti-scarring agent is associated (e.g., coated onto or incorporated into) with an anchoring member (e.g., a fastener, such as a staple or clip) that secures the device to a blood vessel.

As described above, anastomotic connector devices can include a fibrosis-inhibiting agent as a means to improve the clinical efficacy of the device. In another approach, the fibrosis-inhibiting agent can be incorporated into or onto a film or mesh (described in further detail below) that is applied in a perivascular manner to an anastomotic site (e.g., at the junction of a graft vessel and the blood vessel). These films or wraps can be used with any of the anastomotic connector devices described above and, typically, are placed around the outside of the anastomosis at the time of surgery. In other embodiments, the agent may be delivered to the anastomotic site in the form of a spray, paste, gel, or the like. In yet another approach, the fibrosis-inhibiting agent can be incorporated into or onto the graft vessel that is secured to the blood vessel with the connector device.

In yet another aspect, other specialized intravascular devices, such as coronary drug infusion guidewires, such as those available from TherOx, Inc., grafts and balloon over stent devices, such as are described in Wilensky, R. L. (1993) J. Am. Coll. Cardiol.: 21: 185A can also be utilized for local delivery of an anti-fibrosing agent.

As described above, the present invention provides intravascular devices (e.g., anastomotic connectors, stents, drug-delivery balloons, intravascular catheters) that include an anti-scarring agent or a composition that includes an anti-scarring agent. Numerous polymeric and non-polymeric delivery systems for use with intravascular devices have been described above. Methods for incorporating coating fibrosis-inhibiting agents and compositions onto or into intravascular devices include: (a) directly affixing to the intravascular device a fibrosis-inhibiting composition (e.g., by either a spraying process or dipping process as described above, with or without a carrier), (b) directly incorporating into the device a fibrosis-inhibiting composition (e.g., by either a spraying process or dipping process as described above, with or without a carrier (c) by coating the device with a substance such as a hydrogel which will in turn absorb the fibrosis-inhibiting composition), (d) by interweaving fibrosis-inhibiting composition coated thread (or the polymer itself formed into a thread) into the device structure, (e) by inserting the device into a sleeve or mesh which contains or is coated with a fibrosis-inhibiting composition, (f) constructing the device itself or a portion of the device with a fibrosis-inhibiting composition, or (g) by covalently binding the fibrosis-inhibiting agent directly to the device surface or to a linker (small molecule or polymer) that is coated or attached to the device surface. For these devices, the coating process can be performed in such a manner as to (a) coat the external surface of the stent, (b) coat the internal (luminal) surface of the stent or (c) coat all or parts of both the internal and external surfaces of the stent.

The intravascular device (e.g., a stent) may be adapted to release the desired agent at only the distal ends, or along the entire body of the device.

According to the present invention, any fibrosis-inhibiting agent described above can be utilized in the practice of this embodiment. Within one embodiment of the invention, intravascular devices may be adapted to release an agent that inhibits one or more of the four general components of the process of fibrosis (or scarring), including: formation of new blood vessels (angiogenesis), migration and proliferation of connective tissue cells (such as fibroblasts or smooth muscle cells), deposition of extracellular matrix (ECM), and remodeling (maturation and organization of the fibrous tissue). By inhibiting one or more of the components of fibrosis (or scarring), the overgrowth of granulation tissue may be inhibited or reduced.

As intravascular devices are made in a variety of configurations and sizes, the exact dose administered will vary with device size, surface area and design. However, certain principles can be applied in the application of this art. Drug dose can be calculated as a function of dose per unit area (of the portion of the device being coated), total drug dose administered, and appropriate surface concentrations of active drug can be determined. Drugs are to be used at concentrations that range from several times more than to 10%, 5%, or even less than 1% of the concentration typically used in a single chemotherapeutic systemic dose application. Preferably, the drug is released in effective concentrations for a period ranging from 1-90 days.

Several examples of agents for use in intravascular devices include the following: cell cycle inhibitors including (A) anthracyclines (e.g., doxorubicin and mitoxantrone), (B) taxanes (e.g., paclitaxel, TAXOTERE and docetaxel), and (C) podophyllotoxins (e.g., etoposide); (D) immunomodulators (e.g., sirolimus, everolimus, tacrolimus); (E) heat shock protein 90 antagonists (e.g., geldanamycin); (F) HMGCoA reductase inhibitors (e.g., simvastatin); (G) inosine monophosphate dehydrogenase inhibitors (e.g., mycophenolic acid, 1-alpha-25 dihydroxy vitamin D₃); (H)NF kappa B inhibitors (e.g., Bay 11-7082); (I) antimycotic agents (e.g., sulconizole), (J) p38 MAP kinase inhibitors (e.g., SB202190), and (K) angiogenesis inhibitors (e.g., halofuginone bromide), as well as analogues and derivatives of the aforementioned.

Regardless of the method of application of the drug to the intravascular device, the exemplary anti-fibrosing agents, used alone or in combination, should be administered under the following dosing guidelines. The total amount (dose) of anti-scarring agent in or on the device may be in the range of about 0.01 μg-10 μg, or 10 μg-10 mg, or 10 mg-250 mg, or 250 mg-1000 mg, or 1000 mg-2500 mg. The dose (amount) of anti-scarring agent per unit area of device surface to which the agent is applied may be in the range of about 0.01 μg/mm²-1 μg/mm², or 1 μg/mm²-10 μg/mm², or 10 μg/mm²-250 μg/mm², 250 μg/mm²-1000 μg/mm², or 1000 μg/mm²-2500 μg/mm².

Provided below are exemplary dosage ranges for various anti-scarring agents that can be used in conjunction with intravascular devices in accordance with the invention. A) Cell cycle inhibitors including doxorubicin and mitoxantrone. Doxorubicin analogues and derivatives thereof: total dose not to exceed 25 mg (range of 0.1 μg to 25 mg); preferred 1 μg to 5 mg. The dose per unit area of 0.01 μg-100 μg per mm²; preferred dose of 0.1 μg/mm²-10 μg/mm². Minimum concentration of 10⁻⁸-10−4 M of doxorubicin is to be maintained on the device surface. Mitoxantrone and analogues and derivatives thereof: total dose not to exceed 5 mg (range of 0.01 μg to 5 mg); preferred 0.1 μg to 1 mg. The dose per unit area of the device of 0.01 μg-20 μg per mm²; preferred dose of 0.05 μg/mm²-3 μg/mm². Minimum concentration of 10⁻⁸-10⁻⁴ M of mitoxantrone is to be maintained on the device surface. B) Cell cycle inhibitors including paclitaxel and analogues and derivatives (e.g., docetaxel) thereof: total dose not to exceed 10 mg (range of 0.1 μg to 10 mg); preferred 1 μg to 3 mg. The dose per unit area of the device of 0.1 μg-10 μg per mm²; preferred dose of 0.25 μg/mm²-5 μg/mm². Minimum concentration of 10⁻⁸-10⁻⁴ M of paclitaxel is to be maintained on the device surface. (C) Cell cycle inhibitors such as podophyllotoxins (e.g., etoposide): total dose not to exceed 10 mg (range of 0.1 μg to 10 mg); preferred 1 μg to 3 mg. The dose per unit area of the device of 0.1 μg-10 μg per mm²; preferred dose of 0.25 μg/mm²-5 μg/mm². Minimum concentration of 10⁻⁸-10⁻⁴ M of etoposide is to be maintained on the device surface. (D) Immunomodulators including sirolimus and everolimus. Sirolimus (i.e., rapamycin, RAPAMUNE): Total dose not to exceed 10 mg (range of 0.1 μg to 10 mg); preferred 10 μg to 1 mg. The dose per unit area of 0.1 μg-100 μg per mm²; preferred dose of 0.5 μg/mm²-10 μg/mm². Minimum concentration of 10⁻⁸-10⁻⁴ M is to be maintained on the device surface. Everolimus and derivatives and analogues thereof: Total dose should not exceed 10 mg (range of 0.1 μg to 10 mg); preferred 10 μg to 1 mg. The dose per unit area of 0.1 μg-100 μg per mm² of surface area; preferred dose of 0.3 μg/mm²-10 μg/mm². Minimum concentration of 10⁻⁸-10⁻⁴ M of everolimus is to be maintained on the device surface. (E) Heat shock protein 90 antagonists (e.g., geldanamycin) and analogues and derivatives thereof: total dose not to exceed 20 mg (range of 0.1 μg to 20 mg); preferred 1 μg to 5 mg. The dose per unit area of the device of 0.1 μg-10 μg per mm²; preferred dose of 0.25 μg/mm²-5 μg/mm². Minimum concentration of 10⁻⁸-10⁻⁴ M of geldanamycin is to be maintained on the device surface. (F) HMGCoA reductase inhibitors (e.g., simvastatin) and analogues and derivatives thereof: total dose not to exceed 2000 mg (range of 10.0 μg to 2000 mg); preferred 10 μg to 300 mg. The dose per unit area of the device of 1.0 μg-1000 μg per mm²; preferred dose of 2.5 μg/mm²-500 μg/mm². Minimum concentration of 10⁻⁸-10⁻³ M of simvastatin is to be maintained on the device surface. (G) Inosine monophosphate dehydrogenase inhibitors (e.g., mycophenolic acid, 1-alpha-25 dihydroxy vitamin D₃) and analogues and derivatives thereof: total dose not to exceed 2000 mg (range of 10.0 μg to 2000 mg); preferred 10 μg to 300 mg. The dose per unit area of the device of 1.0 μg-1000 μg per mm²; preferred dose of 2.5 μg/mm²-500 μg/mm². Minimum concentration of 10⁻⁸-10⁻³ M of mycophenolic acid is to be maintained on the device surface. (H) NF kappa B inhibitors (e.g., Bay 11-7082) and analogues and derivatives thereof: total dose not to exceed 200 mg (range of 1.0 μg to 200 mg); preferred 1 μg to 50 mg. The dose per unit area of the device of 1.0 μg-100 μg per mm²; preferred dose of 2.5 μg/mm²-50 μg/mm². Minimum concentration of 10⁻⁸-10⁻⁴ M of Bay 11-7082 is to be maintained on the device surface. (I) Antimycotic agents (e.g., sulconizole) and analogues and derivatives thereof: total dose not to exceed 2000 mg (range of 10.0 μg to 2000 mg); preferred 10 μg to 300 mg. The dose per unit area of the device of 1.0 μg-1000 μg per mm²; preferred dose of 2.5 μg/mm²-500 μg/mm². Minimum concentration of 10⁻⁸-10⁻³ M of sulconizole is to be maintained on the device surface. (J) p38 MAP Kinase Inhibitors (e.g., SB202190) and analogues and derivatives thereof: total dose not to exceed 2000 mg (range of 10.0 μg to 2000 mg); preferred 10 μg to 300 mg. The dose per unit area of the device of 1.0 μg-1000 μg per mm²; preferred dose of 2.5 μg/mm²-500 μg/mm². Minimum concentration of 10⁻⁸-10⁻³ M of SB202190 is to be maintained on the device surface. (K) anti-angiogenic agents (e.g., halofuginone bromide) and analogues and derivatives thereof: total dose not to exceed 10 mg (range of 0.1 μg to 10 mg); preferred 1 μg to 3 mg. The dose per unit area of the device of 0.1 μg-10 μg per mm²; preferred dose of 0.25 μg/mm²-5 μg/mm². Minimum concentration of 10⁻⁸-10⁻⁴ M of halofuginone bromide is to be maintained on the device surface.

In addition to those described above (e.g., sirolimus, everolimus, and tacrolimus), several other examples of immunomodulators and appropriate dosages ranges for use with intravascular devices include the following: (A) Biolimus and derivatives and analogues thereof: Total dose should not exceed 10 mg (range of 0.1 μg to 10 mg); preferred 10 μg to 1 mg. The dose per unit area of 0.1 μg-100 μg per mm² of surface area; preferred dose of 0.3 μg/mm²-10 μg/mm². Minimum concentration of 10⁻⁸-10⁻⁴ M of everolimus is to be maintained on the device surface. (B) Tresperimus and derivatives and analogues thereof: Total dose should not exceed 10 mg (range of 0.1 μg to 10 mg); preferred 10 μg to 1 mg. The dose per unit area of 0.1 μg-100 μg per mm² of surface area; preferred dose of 0.3 μg/mm²-10 μg/mm². Minimum concentration of 10⁻⁸-10⁻⁴ M of tresperimus is to be maintained on the device surface. (C) Auranofin and derivatives and analogues thereof: Total dose should not exceed 10 mg (range of 0.1 μg to 10 mg); preferred 10 μg to 1 mg. The dose per unit area of 0.1 μg-100 μg per mm² of surface area; preferred dose of 0.3 μg/mm²-10 μg/mm². Minimum concentration of 10⁻⁸-10⁻⁴ M of auranofin is to be maintained on the device surface. (D) 27-0-Demethylrapamycin and derivatives and analogues thereof: Total dose should not exceed 10 mg (range of 0.1 μg to 10 mg); preferred 10 μg to 1 mg. The dose per unit area of 0.1 μg-100 μg per mm² of surface area; preferred dose of 0.3 μg/mm²-10 μg/mm². Minimum concentration of 10⁻⁸-10⁻⁴ M of 27-0-Demethylrapamycin is to be maintained on the device surface. (E) Gusperimus and derivatives and analogues thereof: Total dose should not exceed 10 mg (range of 0.1 μg to 10 mg); preferred 10 μg to 1 mg. The dose per unit area of 0.1 μg-100 μg per mm² of surface area; preferred dose of 0.3 μg/mm²-10 μg/mm². Minimum concentration of 10⁻⁸-10⁻⁴ M of gusperimus is to be maintained on the device surface. (F) Pimecrolimus and derivatives and analogues thereof: Total dose should not exceed 10 mg (range of 0.1 μg to 10 mg); preferred 10 μg to 1 mg. The dose per unit area of 0.1 μg-100 μg per mm² of surface area; preferred dose of 0.3 μg/mm²-10 μg/mm². Minimum concentration of 10⁻⁸-10⁻⁴ M of pimecrolimus is to be maintained on the device surface and (G) ABT-578 and analogues and derivatives thereof: Total dose should not exceed 10 mg (range of 0.1 μg to 10 mg); preferred 10 μg to 1 mg. The dose per unit area of 0.1 μg-100 μg per mm² of surface area; preferred dose of 0.3 μg/mm²-10 μg/mm². Minimum concentration of 10⁻⁸-10⁻⁴ M of ABT-578 is to be maintained on the device surface.

Gastrointestinal Stents

The present invention provides for the combination of a drug and a gastrointestinal (GI) stent. The term GI stent refers to devices that are located in the gastrointestinal tract including the biliary duct, pancreatic duct, colon, and the esophagus. GI stents are or comprise scaffoldings that are used to treat endoluminal body passageways that have become blocked due to disease or damage, including malignancy or benign disease.

In one aspect, the GI stent may be an esophageal stent used to keep the esophagus open whereby food is able to travel from the mouth to the stomach. For example, the esophageal stent may be composed of a cylindrical supporting mesh inner layer, retaining mesh outer layer and a semi-permeable membrane sandwiched between. See, e.g., U.S. Pat. No. 6,146,416. The esophageal stent may be a radially, self-expanding stent of open weave construction with an elastomeric film formed along the stent to prevent tissue ingrowth and distal cuffs that resist stent migration. See, e.g., U.S. Pat. No. 5,876,448. The esophageal stent may be composed of a flexible wire configuration to form a cylindrical tube with a deformed end portion increased to a larger diameter for anchoring pressure. See, e.g., U.S. Pat. No. 5,876,445. The esophageal stent may be a flexible, self-expandable tubular wall incorporating at least one truncated conical segment along the longitudinal axis. See, e.g., U.S. Pat. No. 6,533,810.

In another aspect, the GI stent may be a biliary stent used to keep the biliary duct open whereby bile is able to drain into the small intestines. For example, the biliary stent may be composed of shape memory alloy. See, e.g., U.S. Pat. No. 5,466,242. The biliary stent may be a plurality of radially extending wings with grooves which project from a helical core. See, e.g., U.S. Pat. Nos. 5,776,160 and 5,486,191.

In another aspect, the GI stent may be a colonic stent. For example, the colonic stent may be a hollow tubular body that may expand radially and be secured to the inner wall of the organ in a release fitting. See, e.g., European Patent Application No. EP1092400A2.

In another aspect, the GI stent may be a pancreatic stent used to keep the pancreatic duct open to facilitate secretion into the small intestines. For example, the pancreatic stent may be composed of a soft biocompatible material which is resiliently compliant which conforms to the duct's curvature and contains perforations that facilitates drainage. See, e.g., U.S. Pat. No. 6,132,471.

GI stents, which may be combined with one or more drugs according to the present invention, include commercially available products, such as the NIR Biliary Stent System and the WALLSTENT Endoprostheses from Boston Scientific Corporation.

In one aspect, the present invention provides GI stents that include an anti-scarring agent or a composition that includes an anti-scarring agent. Numerous polymeric and non-polymeric delivery systems for use in GI stents have been described above.

Methods for incorporating fibrosis-inhibiting agents or fibrosis-inhibiting compositions onto or into the GI stents include: (a) directly affixing to the stent a fibrosis-inhibiting composition (e.g., by either a spraying process or dipping process as described above, with or without a carrier), (b) directly incorporating into the stent a fibrosis-inhibiting composition (e.g., by either a spraying process or dipping process as described above, with or without a carrier), (c) by coating the stent with a substance such as a hydrogel which will in turn absorb the fibrosis-inhibiting composition, (d) by interweaving fibrosis-inhibiting composition coated thread (or the polymer itself formed into a thread) into the stent structure, (e) by inserting the stent into a sleeve or mesh which is comprised of or coated with a fibrosis-inhibiting composition, (f) constructing the stent itself or a portion of the stent with a fibrosis-inhibiting composition, or (g) by covalently binding the fibrosis-inhibiting agent directly to the stent surface or to a linker (small molecule or polymer) that is coated or attached to the stent surface. For these devices, the coating process can be performed in such a manner as to (a) coat the external surface of the stent, (b) coat the internal (luminal) surface of the stent or (c) coat all or parts of both the internal and external surfaces of the stent.

In addition to coating the GI stent with the fibrosis-inhibiting composition, the fibrosis-inhibiting agent can be mixed with the materials that are used to make the device such that the fibrosis-inhibiting agent is incorporated into the final device. This can include the GI stent structure itself, the outer covering or sleeve, if applicable, or both the stent structure and the outer covering or sleeve.

According to the present invention, any fibrosis-inhibiting agent described above can be utilized in the practice of this embodiment. Within one embodiment of the invention, GI stents may be adapted to release an agent that inhibits one or more of the four general components of the process of fibrosis (or scarring), including: formation of new blood vessels (angiogenesis), migration and proliferation of connective tissue cells (such as fibroblasts or smooth muscle cells), deposition of extracellular matrix (ECM), and remodeling (maturation and organization of the fibrous tissue). By inhibiting one or more of the components of fibrosis (or scarring), the overgrowth of granulation tissue may be inhibited or reduced.

As GI stents are made in a variety of configurations and sizes, the exact dose administered will vary with device size, surface area and design. However, certain principles can be applied in the application of this art. Drug dose can be calculated as a function of dose per unit area (of the portion of the device being coated), total dose administered, and appropriate surface concentrations of active drug can be determined. Drugs are to be used at concentrations that range from several times more than to 10%, 5%, or even less than 1% of the concentration typically used in a single chemotherapeutic systemic dose application. Preferably, the drug is released in effective concentrations for a period ranging from 1-90 days.

Several examples of scarring agents for use in GI stents include the following: cell cycle inhibitors including (A) anthracyclines (e.g., doxorubicin and mitoxantrone), (B) taxanes (e.g., paclitaxel, TAXOTERE and docetaxel), and (C) podophyllotoxins (e.g., etoposide); (D) immunomodulators (e.g., sirolimus, everolimus, tacrolimus); (E) heat shock protein 90 antagonists (e.g., geldanamycin); (F) HMGCoA reductase inhibitors (e.g., simvastatin); (G) inosine monophosphate dehydrogenase inhibitors (e.g., mycophenolic acid, 1-alpha-25 dihydroxy vitamin D₃); (H)NF kappa B inhibitors (e.g., Bay 11-7082); (I) antimycotic agents (e.g., sulconizole), (J) p38 MAP kinase inhibitors (e.g., SB202190), and (K) anti-angiogenic agents (e.g., halofuginone bromide), as well as analogues and derivatives of the aforementioned.

Regardless of the method of application of the drug to the GI stent, the exemplary anti-fibrosing agents, used alone or in combination, should be administered under the following dosing guidelines. The total amount (dose) of anti-scarring agent in or on the device may be in the range of about 0.01 μg-10 μg, or 10 μg-10 mg, or 10 mg-250 mg, or 250 mg-1000 mg, or 1000 mg-2500 mg. The dose (amount) of anti-scarring agent per unit area of device surface to which the agent is applied may be in the range of about 0.01 μg/mm²-1 μg/mm², or 1 μg/mm²-10 μg/mm², or 10 μg/mm²-250 μg/mm², 250 μg/mm²-1000 μg/mm², or 1000 μg/mm²-2500 μg/mm².

Provided below are exemplary dosage ranges for various anti-scarring agents that can be used in conjunction with GI stent devices in accordance with the invention. A) Cell cycle inhibitors including doxorubicin and mitoxantrone. Doxorubicin analogues and derivatives thereof: total dose not to exceed 25 mg (range of 0.1 μg to 25 mg); preferred 1 μg to 5 mg. The dose per unit area of 0.01 μg-100 μg per mm²; preferred dose of 0.1 μg/mm²-10 μg/mm². Minimum concentration of 10⁻⁸-10⁻⁴ M of doxorubicin is to be maintained on the device surface. Mitoxantrone and analogues and derivatives thereof: total dose not to exceed 5 mg (range of 0.01 μg to 5 mg); preferred 0.1 μg to 1 mg. The dose per unit area of the device of 0.01 μg-20 μg per mm²; preferred dose of 0.05 μg/mm²-3 μg/mm². Minimum concentration of 10⁻⁸-10⁻⁴ M of mitoxantrone is to be maintained on the device surface. B) Cell cycle inhibitors including Paclitaxel and analogues and derivatives (e.g., docetaxel) thereof: total dose not to exceed 10 mg (range of 0.1 μg to 10 mg); preferred 1 μg to 3 mg. The dose per unit area of the device of 0.1 μg-10 μg per mm²; preferred dose of 0.25 μg/mm²-5 μg/mm². Minimum concentration of 10⁻⁸-10⁻⁴ M of paclitaxel is to be maintained on the device surface. (C) Cell cycle inhibitors such as podophyllotoxins (e.g., etoposide): total dose not to exceed 10 mg (range of 0.1 μg to 10 mg); preferred 1 μg to 3 mg. The dose per unit area of the device of 0.1 μg-10 μg per mm²; preferred dose of 0.25 μg/mm²-5 μg/mm². Minimum concentration of 10⁻⁸-10⁻⁴ M of etoposide is to be maintained on the device surface. (D) Immunomodulators including sirolimus and everolimus. Sirolimus (i.e., rapamycin, RAPAMUNE): Total dose not to exceed 10 mg (range of 0.1 μg to 10 mg); preferred 10 μg to 1 mg. The dose per unit area of 0.1 μg-100 μg per mm²; preferred dose of 0.5 μg/mm²-10 μg/mm². Minimum concentration of 10⁻⁸-10⁻⁴ M is to be maintained on the device surface. Everolimus and derivatives and analogues thereof: Total dose should not exceed 10 mg (range of 0.1 μg to 10 mg); preferred 10 μg to 1 mg. The dose per unit area of 0.1 μg-100 μg per mm² of surface area; preferred dose of 0.3 μg/mm²-10 μg/mm². Minimum concentration of 10⁻⁸-10⁻⁴ M of everolimus is to be maintained on the device surface. (E) Heat shock protein 90 antagonists (e.g., geldanamycin) and analogues and derivatives thereof: total dose not to exceed 20 mg (range of 0.1 μg to 20 mg); preferred 1 μg to 5 mg. The dose per unit area of the device of 0.1 μg-10 μg per mm²; preferred dose of 0.25 μg/mm²-5 μg/mm². Minimum concentration of 10⁻⁸-10⁻⁴ M of geldanamycin is to be maintained on the device surface. (F) HMGCoA reductase inhibitors (e.g., simvastatin) and analogues and derivatives thereof: total dose not to exceed 2000 mg (range of 10.0 μg to 2000 mg); preferred 10 μg to 300 mg. The dose per unit area of the device of 1.0 μg-1000 μg per mm²; preferred dose of 2.5 μg/mm²-500 μg/mm². Minimum concentration of 10⁻⁸-10⁻³ M of simvastatin is to be maintained on the device surface. (G) Inosine monophosphate dehydrogenase inhibitors (e.g., mycophenolic acid, 1-alpha-25 dihydroxy vitamin D₃) and analogues and derivatives thereof: total dose not to exceed 2000 mg (range of 10.0 μg to 2000 mg); preferred 10 μg to 300 mg. The dose per unit area of the device of 1.0 μg-1000 μg per mm²; preferred dose of 2.5 μg/mm²-500 μg/mm². Minimum concentration of 10⁻⁸-10⁻³ M of mycophenolic acid is to be maintained on the device surface. (H)NF kappa B inhibitors (e.g., Bay 11-7082) and analogues and derivatives thereof: total dose not to exceed 200 mg (range of 1.0 μg to 200 mg); preferred 1 μg to 50 mg. The dose per unit area of the device of 1.0 μg-100 μg per mm²; preferred dose of 2.5 μg/mm²-50 μg/mm². Minimum concentration of 10⁻⁸-10⁻⁴ M of Bay 11-7082 is to be maintained on the device surface. (I) Antimycotic agents (e.g., sulconizole) and analogues and derivatives thereof: total dose not to exceed 2000 mg (range of 10.0 μg to 2000 mg); preferred 10 μg to 300 mg. The dose per unit area of the device of 1.0 μg-1000 μg per mm²; preferred dose of 2.5 μg/mm²-500 μg/mm². Minimum concentration of 10⁻⁸-10⁻³ M of sulconizole is to be maintained on the device surface. (J) p38 MAP kinase inhibitors (e.g., SB202190) and analogues and derivatives thereof: total dose not to exceed 2000 mg (range of 10.0 μg to 2000 mg); preferred 10 μg to 300 mg. The dose per unit area of the device of 1.0 μg-1000 μg per mm²; preferred dose of 2.5 μg/mm²-500 μg/mm². Minimum concentration of 10⁻⁸-10⁻³ M of SB202190 is to be maintained on the device surface. K) Anti-angiogenic agents (e.g., halofuginone bromide) and analogues and derivatives thereof: total dose not to exceed 10 mg (range of 0.1 μg to 10 mg); preferred 1 μg to 3 mg. The dose per unit area of the device of 0.1 μg-10 μg per mm²; preferred dose of 0.25 μg/mm²-5 μg/mm². Minimum concentration of 10⁻⁸-10⁻⁴ M of halofuginone bromide is to be maintained on the device surface.

In addition to those described above (e.g., sirolimus, everolimus, and tacrolimus), several other examples of immunomodulators and appropriate dosages ranges for use with gastrointestinal stent devices include the following: (A) Biolimus and derivatives and analogues thereof: Total dose should not exceed 10 mg (range of 0.1 μg to 10 mg); preferred 10 μg to 1 mg. The dose per unit area of 0.1 μg-100 μg per mm² of surface area; preferred dose of 0.3 μg/mm²-10 μg/mm². Minimum concentration of 10⁻⁸-10⁻⁴ M of everolimus is to be maintained on the device surface. (B) Tresperimus and derivatives and analogues thereof: Total dose should not exceed 10 mg (range of 0.1 μg to 10 mg); preferred 10 μg to 1 mg. The dose per unit area of 0.1 μg-100 μg per mm² of surface area; preferred dose of 0.3 μg/mm²-10 μg/mm². Minimum concentration of 10⁻⁸-10⁻⁴ M of tresperimus is to be maintained on the device surface. (C) Auranofin and derivatives and analogues thereof: Total dose should not exceed 10 mg (range of 0.1 μg to 10 mg); preferred 10 μg to 1 mg. The dose per unit area of 0.1 μg-100 μg per mm² of surface area; preferred dose of 0.3 μg/mm²-10 μg/mm². Minimum concentration of 10⁻⁸-10⁻⁴ M of auranofin is to be maintained on the device surface. (D) 27-0-Demethylrapamycin and derivatives and analogues thereof: Total dose should not exceed 10 mg (range of 0.1 μg to 10 mg); preferred 10 μg to 1 mg. The dose per unit area of 0.1 μg-100 μg per mm² of surface area; preferred dose of 0.3 μg/mm²-10 μg/mm². Minimum concentration of 10⁻⁸-10⁻⁴ M of 27-0-Demethylrapamycin is to be maintained on the device surface. (E) Gusperimus and derivatives and analogues thereof: Total dose should not exceed 10 mg (range of 0.1 μg to 10 mg); preferred 10 μg to 1 mg. The dose per unit area of 0.1 μg-100 μg per mm² of surface area; preferred dose of 0.3 μg/mm²-10 μg/mm². Minimum concentration of 10⁻⁸-10⁻⁴ M of gusperimus is to be maintained on the device surface. (F) Pimecrolimus and derivatives and analogues thereof: Total dose should not exceed 10 mg (range of 0.1 μg to 10 mg); preferred 10 μg to 1 mg. The dose per unit area of 0.1 μg-100 μg per mm² of surface area; preferred dose of 0.3 μg/mm²-10 μg/mm². Minimum concentration of 10⁻⁸-10⁻⁴ M of pimecrolimus is to be maintained on the device surface and (G) ABT-578 and analogues and derivatives thereof: Total dose should not exceed 10 mg (range of 0.1 μg to 10 mg); preferred 10 μg to 1 mg. The dose per unit area of 0.1 μg-100 μg per mm² of surface area; preferred dose of 0.3 μg/mm²-10 μg/mm². Minimum concentration of 10⁻⁸-10⁻⁴ M of ABT-578 is to be maintained on the device surface.

Tracheal and Bronchial Stents

The present invention provides for the combination of an anti-scarring agent and a tracheal or bronchial stent device.

Representative examples of tracheal or bronchial stents that can benefit from being coated with or having incorporated therein, a fibrosis-inhibiting agent include tracheal stents or bronchial stents, including metallic and polymeric tracheal or bronchial stents and tracheal or bronchial stents that have an external covering (e.g., polyurethane, poly(ethylene terephthalate), PTFE, or silicone rubber).

Tracheal and bronchial stents may be, for example, composed of an elastic plastic shaft with metal clasps that expands to form a lumen along the axis for opening the diseased portion of the trachea and having three sections to emulate the natural shape of the trachea. See, e.g., U.S. Pat. No. 5,480,431. The tracheal/bronchial stent may be a T-shaped tube having a tracheotomy tubular portion that projects outwardly through a tracheotomy orifice which is configured to close and form a fluid seal. See, e.g., U.S. Pat. Nos. 5,184,610 and 3,721,233. The tracheal/bronchial stent may be composed of a flexible, synthetic polymeric resin with a tracheotomy tube mounted on the wall with a bifurcated bronchial end that is configured in a T-Y shape with specific curves at the intersections to minimize tissue damage. See, e.g., U.S. Pat. No. 4,795,465. The tracheal/bronchial stent may be a scaffolding configured to be substantially cylindrical with a shape-memory frame having geometrical patterns and having a coating of sufficient thickness to prevent epithelialization. See, e.g., U.S. Patent Application Publication No. 2003/0024534A1.

Tracheal/bronchial stents, which may be combined with one or more agents according to the present invention, include commercially available products, such as the WALLSTENT Tracheobronchial Endoprostheses and ULTRAFLEX Tracheobronchial Stent Systems from Boston Scientific Corporation and the DUMON Tracheobronchial Silicone Stents from Bryan Corporation (Woburn, Mass.).

In one aspect, the present invention provides tracheal and bronchial stents that include an anti-scarring agent or a composition that includes an anti-scarring agent. Numerous polymeric and non-polymeric delivery systems for use in tracheal and bronchial stents have been described above. Methods for incorporating fibrosis-inhibiting agents or fibrosis-inhibiting compositions onto or into the tracheal or bronchial stents include: (a) directly affixing to the stent a fibrosis-inhibiting composition (e.g., by either a spraying process or dipping process as described above, with or without a carrier), (b) directly incorporating into the stent a fibrosis-inhibiting composition (e.g., by either a spraying process or dipping process as described above, with or without a carrier (c) by coating the stent with a substance such as a hydrogel which will in turn absorb the fibrosis-inhibiting composition), (d) by interweaving fibrosis-inhibiting composition coated thread (or the polymer itself formed into a thread) into the stent structure, (e) by inserting the stent into a sleeve or mesh which is comprised of or coated with a fibrosis-inhibiting composition, (f) constructing the stent itself or a portion of the stent with a fibrosis-inhibiting composition, or (g) by covalently binding the fibrosis-inhibiting agent directly to the stent surface or to a linker (small molecule or polymer) that is coated or attached to the stent surface. For these devices, the coating process can be performed in such a manner as to (a) coat the external surface of the stent, (b) coat the internal (luminal) surface of the stent or (c) coat all or parts of both the internal and external surfaces of the stent.

In addition to coating the device with the fibrosis-inhibiting composition, the fibrosis-inhibiting agent can be mixed with the materials that are used to make the device such that the fibrosis-inhibiting agent is incorporated into the final device. This can include the stent structure itself, the outer covering or sleeve, if applicable, or both the stent structure and the outer covering or sleeve.

According to the present invention, any fibrosis-inhibiting agent described above can be utilized in the practice of this embodiment. Within one embodiment of the invention, tracheal and bronchial stents may be adapted to release an agent that inhibits one or more of the four general components of the process of fibrosis (or scarring), including: formation of new blood vessels (angiogenesis), migration and proliferation of connective tissue cells (such as fibroblasts or smooth muscle cells), deposition of extracellular matrix (ECM), and remodeling (maturation and organization of the fibrous tissue). By inhibiting one or more of the components of fibrosis (or scarring), the overgrowth of granulation tissue may be inhibited or reduced.

As tracheal and bronchial stents are made in a variety of configurations and sizes, the exact dose administered will vary with device size, surface area and design. However, certain principles can be applied in the application of this art. Drug dose can be calculated as a function of dose per unit area (of the portion of the device being coated), total dose administered, and appropriate surface concentrations of active drug can be determined. Drugs are to be used at concentrations that range from several times more than to 10%, 5%, or even less than 1% of the concentration typically used in a single chemotherapeutic systemic dose application. Preferably, the drug is released in effective concentrations for a period ranging from 1-90 days.

Several fibrosis-inhibiting agents for use in tracheal and bronchial stents include the following: cell cycle inhibitors including (A) anthracyclines (e.g., doxorubicin and mitoxantrone), (B) taxanes (e.g., paclitaxel, TAXOTERE and docetaxel), and (C) podophyllotoxins (e.g., etoposide); (D) immunomodulators (e.g., sirolimus, everolimus, tacrolimus); (E) heat shock protein 90 antagonists (e.g., geldanamycin); (F) HMGCoA reductase inhibitors (e.g., simvastatin); (G) inosine monophosphate dehydrogenase inhibitors (e.g., mycophenolic acid, 1-alpha-25 dihydroxy vitamin D₃); (H)NF kappa B inhibitors (e.g., Bay 11-7082); (I) antimycotic agents (e.g., sulconizole), (J) p38 MAP kinase inhibitors (e.g., SB202190), and (K) and anti-angiogenesis agents (e.g., halofuginone bromide), as well as analogues and derivatives of the aforementioned.

Regardless of the method of application of the drug to the tracheal or bronchial stent, the exemplary anti-fibrosing agents, used alone or in combination, should be administered under the following dosing guidelines. The total amount (dose) of anti-scarring agent in or on the device may be in the range of about 0.01 μg-10 μg, or 10 μg-10 mg, or 10 mg-250 mg, or 250 mg-1000 mg, or 1000 mg-2500 mg. The dose (amount) of anti-scarring agent per unit area of device surface to which the agent is applied may be in the range of about 0.01 μg/mm²-1 μg/mm², or 1 μg/mm²-10 μg/mm², or 10 μg/mm²-250 μg/mm², 250 μg/mm²-1000 μg/mm², or 1000 μg/mm²-2500 μg/mm².

Provided below are exemplary dosage ranges for various anti-scarring agents that can be used in conjunction with tracheal and bronchial stent devices in accordance with the invention. A) Cell cycle inhibitors including doxorubicin and mitoxantrone. Doxorubicin analogues and derivatives thereof: total dose not to exceed 25 mg (range of 0.1 μg to 25 mg); preferred 1 μg to 5 mg. The dose per unit area of 0.01 μg-100 μg per mm²; preferred dose of 0.1 μg/mm²-10 μg/mm². Minimum concentration of 10⁻⁸-10⁻⁴ M of doxorubicin is to be maintained on the device surface. Mitoxantrone and analogues and derivatives thereof: total dose not to exceed 5 mg (range of 0.01 μg to 5 mg); preferred 0.1 μg to 1 mg. The dose per unit area of the device of 0.01 μg-20 μg per mm²; preferred dose of 0.05 μg/mm²-3 μg/mm². Minimum concentration of 10⁻⁸-10⁻⁴ M of mitoxantrone is to be maintained on the device surface. B) Cell cycle inhibitors including paclitaxel and analogues and derivatives (e.g., docetaxel) thereof: total dose not to exceed 10 mg (range of 0.1 μg to 10 mg); preferred 1 μg to 3 mg. The dose per unit area of the device of 0.1 μg-10 μg per mm²; preferred dose of 0.25 μg/mm²-5 μg/mm². Minimum concentration of 10⁻⁸-10⁻⁴ M of paclitaxel is to be maintained on the device surface. (C) Cell cycle inhibitors such as podophyllotoxins (e.g., etoposide): total dose not to exceed 10 mg (range of 0.1 μg to 10 mg); preferred 1 μg to 3 mg. The dose per unit area of the device of 0.1 μg-10 μg per mm²; preferred dose of 0.25 μg/mm²-5 μg/mm². Minimum concentration of 10⁻⁸-10⁻⁴ M of etoposide is to be maintained on the device surface. (D) Immunomodulators including sirolimus and everolimus. Sirolimus (i.e., rapamycin, RAPAMUNE): Total dose not to exceed 10 mg (range of 0.1 μg to 10 mg); preferred 10 μg to 1 mg. The dose per unit area of the device 0.1 μg-100 μg per mm²; preferred dose of 0.5 μg/mm²-10 μg/mm². Minimum concentration of 10⁻⁸-10⁻⁴ M is to be maintained on the device surface. Everolimus and derivatives and analogues thereof: Total dose should not exceed 10 mg (range of 0.1 μg to 10 mg); preferred 10 μg to 1 mg. The dose per unit area of 0.1 μg-100 μg per mm² of surface area; preferred dose of 0.3 μg/mm²-10 μg/mm². Minimum concentration of 10⁻⁸-10⁻⁴ M of everolimus is to be maintained on the device surface. (E) Heat shock protein 90 antagonists (e.g., geldanamycin) and analogues and derivatives thereof: total dose not to exceed 20 mg (range of 0.1 μg to 20 mg); preferred 1 μg to 5 mg. The dose per unit area of the device of 0.1 μg-10 μg per mm²; preferred dose of 0.25 μg/mm²-5 μg/mm². Minimum concentration of 10⁻⁸-10⁻⁴ M of geldanamycin is to be maintained on the device surface. (F) HMGCoA reductase inhibitors (e.g., simvastatin) and analogues and derivatives thereof: total dose not to exceed 2000 mg (range of 10.0 μg to 2000 mg); preferred 10 μg to 300 mg. The dose per unit area of the device of 1.0 μg-1000 μg per mm²; preferred dose of 2.5 μg/mm²-500 μg/mm². Minimum concentration of 10⁻⁸-10⁻³ M of simvastatin is to be maintained on the device surface. (G) Inosine monophosphate dehydrogenase inhibitors (e.g., mycophenolic acid, 1-alpha-25 dihydroxy vitamin D₃) and analogues and derivatives thereof: total dose not to exceed 2000 mg (range of 10.0 μg to 2000 mg); preferred 10 μg to 300 mg. The dose per unit area of the device of 1.0 μg-1000 μg per mm²; preferred dose of 2.5 μg/mm²-500 μg/mm². Minimum concentration of 10⁻⁸-10⁻³ M of mycophenolic acid is to be maintained on the device surface. (H)NF kappa B inhibitors (e.g., Bay 11-7082) and analogues and derivatives thereof: total dose not to exceed 200 mg (range of 1.0 μg to 200 mg); preferred 1 μg to 50 mg. The dose per unit area of the device of 1.0 μg-100 μg per mm²; preferred dose of 2.5 μg/mm²-50 μg/mm². Minimum concentration of 10⁻⁸-10⁻⁴ M of Bay 11-7082 is to be maintained on the device surface. (I) Antimycotic agents (e.g., sulconizole) and analogues and derivatives thereof: total dose not to exceed 2000 mg (range of 10.0 μg to 2000 mg); preferred 10 μg to 300 mg. The dose per unit area of the device of 1.0 μg-1000 μg per mm²; preferred dose of 2.5 μg/mm²-500 μg/mm². Minimum concentration of 10⁻⁸-10⁻³ M of sulconizole is to be maintained on the device surface. (J) p38 MAP kinase inhibitors (e.g., SB202190) and analogues and derivatives thereof: total dose not to exceed 2000 mg (range of 10.0 μg to 2000 mg); preferred 10 μg to 300 mg. The dose per unit area of the device of 1.0 μg-1000 μg per mm²; preferred dose of 2.5 μg/mm²-500 μg/mm². Minimum concentration of 10⁻⁸-10⁻³ M of SB202190 is to be maintained on the device surface. (K) Anti-angiogenic agents (e.g., halofuginone bromide) and analogues and derivatives thereof: total dose not to exceed 10 mg (range of 0.1 μg to 10 mg); preferred 1 μg to 3 mg. The dose per unit area of the device of 0.1 μg-10 μg per mm²; preferred dose of 0.25 μg/mm²-5 μg/mm². Minimum concentration of 10⁻⁸-10⁻⁴ M of halofuginone bromide is to be maintained on the device surface.

In addition to those described above (e.g., sirolimus, everolimus, and tacrolimus), several other examples of immunomodulators and appropriate dosages ranges for use with intravascular devices include the following: (A) Biolimus and derivatives and analogues thereof: Total dose should not exceed 10 mg (range of 0.1 μg to 10 mg); preferred 10 μg to 1 mg. The dose per unit area of 0.1 μg-100 μg per mm² of surface area; preferred dose of 0.3 μg/mm²-10 μg/mm². Minimum concentration of 10⁻⁸-10⁻⁴ M of everolimus is to be maintained on the device surface. (B) Tresperimus and derivatives and analogues thereof: Total dose should not exceed 10 mg (range of 0.1 μg to 10 mg); preferred 10 μg to 1 mg. The dose per unit area of 0.1 μg-100 μg per mm² of surface area; preferred dose of 0.3 μg/mm²-10 μg/mm². Minimum concentration of 10⁻⁸-10⁻⁴ M of tresperimus is to be maintained on the device surface. (C) Auranofin and derivatives and analogues thereof: Total dose should not exceed 10 mg (range of 0.1 μg to 10 mg); preferred 10 μg to 1 mg. The dose per unit area of 0.1 μg-100 μg per mm² of surface area; preferred dose of 0.3 μg/mm²-10 μg/mm². Minimum concentration of 10⁻⁸-10⁻⁴ M of auranofin is to be maintained on the device surface. (D) 27-0-Demethylrapamycin and derivatives and analogues thereof: Total dose should not exceed 10 mg (range of 0.1 μg to 10 mg); preferred 10 μg to 1 mg. The dose per unit area of 0.1 μg-100 μg per mm² of surface area; preferred dose of 0.3 μg/mm²-10 μg/mm². Minimum concentration of 10⁻⁸-10⁻⁴ M of 27-0-Demethylrapamycin is to be maintained on the device surface. (E) Gusperimus and derivatives and analogues thereof: Total dose should not exceed 10 mg (range of 0.1 μg to 10 mg); preferred 10 μg to 1 mg. The dose per unit area of 0.1 μg-100 μg per mm² of surface area; preferred dose of 0.3 μg/mm²-10 μg/mm². Minimum concentration of 10⁻⁸-10⁻⁴ M of gusperimus is to be maintained on the device surface. (F) Pimecrolimus and derivatives and analogues thereof: Total dose should not exceed 10 mg (range of 0.1 μg to 10 mg); preferred 10 μg to 1 mg. The dose per unit area of 0.1 μg-100 μg per mm² of surface area; preferred dose of 0.3 μg/mm²-10 μg/mm². Minimum concentration of 10⁻⁸-10⁻⁴ M of pimecrolimus is to be maintained on the device surface and (G) ABT-578 and analogues and derivatives thereof: Total dose should not exceed 10 mg (range of 0.1 μg to 10 mg); preferred 10 μg to 1 mg. The dose per unit area of 0.1 μg-100 μg per mm² of surface area; preferred dose of 0.3 μg/mm²-10 μg/mm². Minimum concentration of 10⁻⁸-10⁻⁴ M of ABT-578 is to be maintained on the device surface.

Genital-Urinary Stents

The present invention provides for the combination of an anti-scarring agent and genital-urinary (GU) stent device.

Representative examples genital-urinary (GU) stents that can benefit from being coated with or having incorporated therein, a fibrosis-inhibiting agent include ureteric and urethral stents, fallopian tube stents, prostate stents, including metallic and polymeric GU stents and GU stents that have an external covering (e.g., polyurethane, poly(ethylene terephthalate), PTFE or silicone rubber).

In one aspect, genital-urinary stents include ureteric and urethral stents. Ureteral stents are hollow tubes with holes along the sides and coils at either end to prevent migration. Ureteral stents are used to relieve obstructions (caused by stones or malignancy), to facilitate the passage of stones, or to allow healing of ureteral anastomoses or leaks following surgery or trauma. They are placed endoscopically via the bladder or percutaneously via the kidney.

Urethral stents are used for the treatment of recurrent urethral strictures, detruso-external sphincter dyssynergia and bladder outlet obstruction due to benign prostatic hypertrophy. In addition, procedures that are conducted for the prostate, such as external radiation or brachytherapy, may lead to fibrosis due to tissue insult resulting from these procedures. The incidence of urethral stricture in prostate cancer patients treated with external beam radiation is about 2%. Development of urethral stricture may also occur in other conditions such as following urinary catheterization or surgery, which results in damage to the epithelium of the urethra. The clinical manifestation of urinary tract obstruction includes decreased force and caliber of the urinary stream, intermittency, postvoid dribbling, hesitance and nocturia. Complete closure of the urethra can result in numerous problems including eventual kidney failure. To maintain patency in the urethra, urethral stents may be used. The stents are typically self-expanding and composed of metal superalloy, titanium, stainless steel or polyurethane.

For example, the ureteric/urethral stent may be composed of a main catheter body of flexible polymeric material having an enlarged entry end with a hydrophilic tip that dissolves when contacted with body fluids. See, e.g., U.S. Pat. No. 5,401,257. The ureteric/urethral stent may be composed of a multi-sections including a closed section at that the bladder end which does not contain any fluid passageways such that it acts as an anti-reflux device to prevent reflux of urine back into the kidney. See, e.g., U.S. Pat. No. 5,647,843. The ureteric/urethral stent may be composed of a central catheter tube made of shape memory material that forms a stent with a retention coil for anchoring to the ureter. See, e.g., U.S. Pat. No. 5,681,274. The ureteric/urethral stent may be a composed of an elongated flexible tubular stent with preformed set curls at both ends and an elongated tubular rigid extension attached to the distal end which allows the combination function as an externalized ureteral catheter. See, e.g., U.S. Pat. Nos. 5,221,253 and 5,116,309. The ureteric/urethral stent may be composed of an elongated member, a proximal retention structure, and a resilient portion connecting them together, whereby they are all in fluid communication with each other with a slideable portion providing a retracted and expanded position. See, e.g., U.S. Pat. No. 6,685,744. The ureteric/urethral stent may be a hollow cylindrical tube that has a flexible connecting means and locating means that expands and selectively contracts. See, e.g., U.S. Pat. No. 5,322,501. The ureteric/urethral stent may be composed of a stiff polymeric body that affords superior columnar and axial strength for advancement into the ureter, and a softer bladder coil portion for reducing the risk of irritation. See, e.g., U.S. Pat. No. 5,141,502. The ureteric/urethral stent may be composed of an elongated tubular segment that has a pliable wall at the proximal region and a plurality of members that prevent blockage of fluid drainage upon compression. See, e.g., U.S. Pat. No. 6,676,623. The ureteric/urethral stent may be a catheter composed of a conduit which is part of an assembly that allows for non-contaminated insertion into a urinary canal by providing a sealing member that surrounds the catheter 2003/0060807A1.

In another aspect, genital-urinary stents include prostatic stents. For example, the prostatic stent may be composed of two polymeric rings constructed of tubing with a plurality of connecting arm members connecting the rings in a parallel manner. See, e.g., U.S. Pat. No. 5,269,802. The prostatic stent may be composed of thermoplastic material and a circumferential reinforcing helical spring, which provides rigid mechanical support while being flexible to accommodate the natural anatomical bend of the prostatic urethra. See, e.g., U.S. Pat. No. 5,069,169.

In another aspect, genital-urinary stents include fallopian stents and other female genital-urinary devices. For example, the genital-urinary device may be a female urinary incontinence device composed of a vaginal-insertable supporting portion that is resilient and flexible, which is capable of self-support by expansion against the vaginal wall and extending about the urethral orifice. See, e.g., U.S. Pat. No. 3,661,155. The genital-urinary device may be a urinary evacuation device composed of a ovular bulbous concave wall having an opening to a body engaging perimetal edge integral with the wall and an attached tubular member with a pleated body. See, e.g., U.S. Pat. No. 6,041,448.

Genital-urinary stents, which may be combined with one or more agents according to the present invention, include commercially available products, such as the UROLUME Endoprosthesis Stents from American Medical Systems, Inc. (Minnetonka, Minn.), the RELIEVE Prostatic/Urethral Endoscopic Device from InjecTx, Inc. (San Jose, Calif.), the PERCUFLEX Ureteral Stents from Boston Scientific Corporation, and the TARKINGTON Urethral Stents and FIRLIT-KLUGE Urethral Stents from Cook Group Inc (Bloomington, Ind.).

In one aspect, the present invention provides GU stents that include an anti-scarring agent or a composition that includes an anti-scarring agent. Numerous polymeric and non-polymeric delivery systems for use in GU stents have been described above. Methods for incorporating fibrosing agents or fibrosis-inhibiting compositions onto or into the GU stents include: (a) directly affixing to the stent a fibrosis-inhibiting composition (e.g., by either a spraying process or dipping process as described above, with or without a carrier), (b) directly incorporating into the stent a fibrosis-inhibiting composition (e.g., by either a spraying process or dipping process as described above, with or without a carrier), (c) by coating the stent with a substance such as a hydrogel which will in turn absorb the fibrosis-inhibiting composition, (d) by interweaving fibrosis-inhibiting composition coated thread (or the polymer itself formed into a thread) into the stent structure, (e) by inserting the stent into a sleeve or mesh which is comprised of or coated with a fibrosis-inhibiting composition, (f) constructing the stent itself or a portion of the stent with a fibrosis-inhibiting composition, or (g) by covalently binding the fibrosis-inhibiting agent directly to the stent surface or to a linker (small molecule or polymer) that is coated or attached to the stent surface. For these devices, the coating process can be performed in such a manner as to (a) coat the external surface of the stent, (b) coat the internal (luminal) surface of the stent or (c) coat all or parts of both the internal and external surfaces of the stent.

In addition to coating the device with the fibrosis-inhibiting composition, the fibrosis-inhibiting agent can be mixed with the materials that are used to make the device such that the fibrosis-inhibiting agent is incorporated into the final device.

According to the present invention, any fibrosis-inhibiting agent described above can be utilized in the practice of this embodiment. Within one embodiment of the invention, GU stents may be adapted to release an agent that inhibits one or more of the four general components of the process of fibrosis (or scarring), including: formation of new blood vessels (angiogenesis), migration and proliferation of connective tissue cells (such as fibroblasts or smooth muscle cells), deposition of extracellular matrix (ECM), and remodeling (maturation and organization of the fibrous tissue). By inhibiting one or more of the components of fibrosis (or scarring), the overgrowth of granulation tissue may be inhibited or reduced.

As GU stents are made in a variety of configurations and sizes, the exact dose administered will vary with device size, surface area and design. However, certain principles can be applied in the application of this art. Drug dose can be calculated as a function of dose per unit area (of the portion of the device being coated), total dose administered, and appropriate surface concentrations of active drug can be determined. Drugs are to be used at concentrations that range from several times more than to 10%, 5%, or even less than 1% of the concentration typically used in a single chemotherapeutic systemic dose application. Preferably, the drug is released in effective concentrations for a period ranging from 1-90 days.

Several examples of scarring agents for use in GU stents include the following: cell cycle inhibitors including (A) anthracyclines (e.g., doxorubicin and mitoxantrone), (B) taxanes (e.g., paclitaxel, TAXOTERE and docetaxel), and (C) podophyllotoxins (e.g., etoposide); (D) immunomodulators (e.g., sirolimus, everolimus, tacrolimus); (E) heat shock protein 90 antagonists (e.g., geldanamycin); (F) HMGCoA reductase inhibitors (e.g., simvastatin); (G) inosine monophosphate dehydrogenase inhibitors (e.g., mycophenolic acid, 1-alpha-25 dihydroxy vitamin D₃); (H)NF kappa B inhibitors (e.g., Bay 11-7082); (I) antimycotic agents (e.g., sulconizole), (J) p38 MAP kinase inhibitors (e.g., SB202190), and (K) and anti-angiogenesis agents (e.g., halofuginone bromide), as well as analogues and derivatives of the aforementioned.

Regardless of the method of application of the drug to the GU stent, the exemplary anti-fibrosing agents, used alone or in combination, should be administered under the following dosing guidelines. The total amount (dose) of anti-scarring agent in or on the device may be in the range of about 0.01 μg-10 μg, or 10 μg-10 mg, or 10 mg-250 mg, or 250 mg-1000 mg, or 1000 mg-2500 mg. The dose (amount) of anti-scarring agent per unit area of device surface to which the agent is applied may be in the range of about 0.01 μg/mm²-1 μg/mm², or 1 μg/mm²-10 μg/mm², or 10 μg/mm²-250 μg/mm², 250 μg/mm²-1000 μg/mm², or 1000 μg/mm²-2500 μg/mm².

Provided below are exemplary dosage ranges for various anti-scarring agents that can be used in conjunction with GU stent devices in accordance with the invention. A) Cell cycle inhibitors including doxorubicin and mitoxantrone. Doxorubicin analogues and derivatives thereof: total dose not to exceed 25 mg (range of 0.1 μg to 25 mg); preferred 1 μg to 5 mg. The dose per unit area of 0.01 μg-100 μg per mm²; preferred dose of 0.1 μg mm²-10 μg/mm². Minimum concentration of 10⁻⁸-10⁻⁴ M of doxorubicin is to be maintained on the device surface. Mitoxantrone and analogues and derivatives thereof: total dose not to exceed 5 mg (range of 0.01 μg to 5 mg); preferred 0.1 μg to 1 mg. The dose per unit area of the device of 0.01 μg-20 μg per mm²; preferred dose of 0.05 μg/mm²-3 μg/mm². Minimum concentration of 10⁻⁸-10⁻⁴ M of mitoxantrone is to be maintained on the device surface. B) Cell cycle inhibitors including Paclitaxel and analogues and derivatives (e.g., docetaxel) thereof: total dose not to exceed 10 mg (range of 0.1 μg to 10 mg); preferred 1 μg to 3 mg. The dose per unit area of the device of 0.1 μg-10 μg per mm²; preferred dose of 0.25 μg/mm²-5 μg/mm². Minimum concentration of 10⁻⁸-10⁴ M of paclitaxel is to be maintained on the device surface. (C) Cell cycle inhibitors such as podophyllotoxins (e.g., etoposide): total dose not to exceed 10 mg (range of 0.1 μg to 10 mg); preferred 1 μg to 3 mg. The dose per unit area of the device of 0.1 μg-10 μg per mm²; preferred dose of 0.25 μg/mm²-5 μg/mm². Minimum concentration of 10⁻⁸-10⁻⁴ M of etoposide is to be maintained on the device surface. (D) Immunomodulators including sirolimus and everolimus. Sirolimus (i.e., rapamycin, RAPAMUNE): Total dose not to exceed 10 mg (range of 0.1 μg to 10 mg); preferred 10 μg to 1 mg. The dose per unit area of 0.1 μg-100 μg per mm²; preferred dose of 0.5 μg/mm²-10 μg/mm². Minimum concentration of 10⁻⁸-10⁻⁴ M is to be maintained on the device surface. Everolimus and derivatives and analogues thereof: Total dose should not exceed 10 mg (range of 0.1 μg to 10 mg); preferred 10 μg to 1 mg. The dose per unit area of 0.1 μg-100 μg per mm² of surface area; preferred dose of 0.3 μg/mm²-10 μg/mm². Minimum concentration of 10⁻⁸-10⁻⁴ M of everolimus is to be maintained on the device surface. (E) Heat shock protein 90 antagonists (e.g., geldanamycin) and analogues and derivatives thereof: total dose not to exceed 20 mg (range of 0.1 μg to 20 mg); preferred 1 μg to 5 mg. The dose per unit area of the device of 0.1 μg-10 μg per mm²; preferred dose of 0.25 μg/mm²-5 μg/mm². Minimum concentration of 10⁻⁸-10⁻⁴ M of geldanamycin is to be maintained on the device surface. (F) HMGCoA reductase inhibitors (e.g., simvastatin) and analogues and derivatives thereof: total dose not to exceed 2000 mg (range of 10.0 μg to 2000 mg); preferred 10 μg to 300 mg. The dose per unit area of the device of 1.0 μg-1000 μg per mm²; preferred dose of 2.5 μg/mm²-500 μg/mm². Minimum concentration of 10⁻⁸-10⁻³ M of simvastatin is to be maintained on the device surface. (G) Inosine monophosphate dehydrogenase inhibitors (e.g., mycophenolic acid, 1-alpha-25 dihydroxy vitamin D₃) and analogues and derivatives thereof: total dose not to exceed 2000 mg (range of 10.0 μg to 2000 mg); preferred 10 μg to 300 mg. The dose per unit area of the device of 1.0 μg-1000 μg per mm²; preferred dose of 2.5 μg/mm²-500 μg/mm². Minimum concentration of 10⁻⁸-10⁻³ M of mycophenolic acid is to be maintained on the device surface. (H)NF kappa B inhibitors (e.g., Bay 11-7082) and analogues and derivatives thereof: total dose not to exceed 200 mg (range of 1.0 μg to 200 mg); preferred 1 μg to 50 mg. The dose per unit area of the device of 1.0 μg-100 μg per mm²; preferred dose of 2.5 μg/mm²-50 μg/mm². Minimum concentration of 10⁻⁸-10⁻⁴ M of Bay 11-7082 is to be maintained on the device surface. (I) Antimycotic agents (e.g., sulconizole) and analogues and derivatives thereof: total dose not to exceed 2000 mg (range of 10.0 μg to 2000 mg); preferred 10 μg to 300 mg. The dose per unit area of the device of 1.0 μg-1000 μg per mm²; preferred dose of 2.5 μg/mm²-500 μg/mm². Minimum concentration of 10⁻⁸-10⁻³ M of sulconizole is to be maintained on the device surface. (J) p38 MAP kinase inhibitors (e.g., SB202190) and analogues and derivatives thereof: total dose not to exceed 2000 mg (range of 10.0 μg to 2000 mg); preferred 10 μg to 300 mg. The dose per unit area of the device of 1.0 μg-1000 μg per mm²; preferred dose of 2.5 μg/mm²-500 μg/mm². Minimum concentration of 10⁻⁸-10⁻³ M of SB202190 is to be maintained on the device surface. (K) Anti-angiogenic agents (e.g., halofuginone bromide) and analogues and derivatives thereof: total dose not to exceed 10 mg (range of 0.1 μg to 10 mg); preferred 1 μg to 3 mg. The dose per unit area of the device of 0.1 μg-10 μg per mm²; preferred dose of 0.25 μg/mm²-5 μg/mm². Minimum concentration of 10⁻⁸-10⁻⁴ M of halofuginone bromide is to be maintained on the device surface.

In addition to those described above (e.g., sirolimus, everolimus, and tacrolimus), several other examples of immunomodulators and appropriate dosages ranges for use with genital-urinary stent devices include the following: (A) Biolimus and derivatives and analogues thereof: Total dose should not exceed 10 mg (range of 0.1 μg to 10 mg); preferred 10 μg to 1 mg. The dose per unit area of 0.1 μg-100 μg per mm² of surface area; preferred dose of 0.3 μg/mm²-10 μg/mm². Minimum concentration of 10⁻⁸-10⁻⁴ M of everolimus is to be maintained on the device surface. (B) Tresperimus and derivatives and analogues thereof: Total dose should not exceed 10 mg (range of 0.1 μg to 10 mg); preferred 10 μg to 1 mg. The dose per unit area of 0.1 μg-100 μg per mm² of surface area; preferred dose of 0.3 μg/mm²-10 μg/mm². Minimum concentration of 10⁻⁸-10⁻⁴ M of tresperimus is to be maintained on the device surface. (C) Auranofin and derivatives and analogues thereof: Total dose should not exceed 10 mg (range of 0.1 μg to 10 mg); preferred 10 μg to 1 mg. The dose per unit area of 0.1 μg-100 μg per mm² of surface area; preferred dose of 0.3 μg/mm²-10 μg/mm². Minimum concentration of 10⁻⁸-10⁻⁴ M of auranofin is to be maintained on the device surface. (D) 27-0-Demethylrapamycin and derivatives and analogues thereof: Total dose should not exceed 10 mg (range of 0.1 μg to 10 mg); preferred 10 μg to 1 mg. The dose per unit area of 0.1 μg-100 μg per mm² of surface area; preferred dose of 0.3 μg/mm²-10 μg/mm². Minimum concentration of 10⁻⁸-10⁻⁴ M of 27-0-Demethylrapamycin is to be maintained on the device surface. (E) Gusperimus and derivatives and analogues thereof: Total dose should not exceed 10 mg (range of 0.1 μg to 10 mg); preferred 10 μg to 1 mg. The dose per unit area of 0.1 μg-100 μg per mm² of surface area; preferred dose of 0.3 μg/mm²-10 μg/mm². Minimum concentration of 10⁻⁸-10⁻⁴ M of gusperimus is to be maintained on the device surface. (F) Pimecrolimus and derivatives and analogues thereof: Total dose should not exceed 10 mg (range of 0.1 μg to 10 mg); preferred 10 μg to 1 mg. The dose per unit area of 0.1 μg-100 μg per mm² of surface area; preferred dose of 0.3 μg/mm²-10 μg/mm². Minimum concentration of 10⁻⁸-10⁻⁴ M of pimecrolimus is to be maintained on the device surface and (G) ABT-578 and analogues and derivatives thereof: Total dose should not exceed 10 mg (range of 0.1 μg to 10 mg); preferred 10 μg to 1 mg. The dose per unit area of 0.1 μg-100 μg per mm² of surface area; preferred dose of 0.3 μg/mm²-10 μg/mm². Minimum concentration of 10⁻⁸-10⁻⁴ M of ABT-578 is to be maintained on the device surface.

Ear and Nose Stents

The present invention provides for the combination of an anti-scarring agent and an ear-nose-throat (ENT) stent device (e.g., a lacrimal duct stent, Eustachian tube stent, nasal stent, or sinus stent).

The sinuses are four pairs of hollow regions contained in the bones of the skull named after the bones in which they are located (ethmoid, maxillary, frontal and sphenoid). All are lined by respiratory mucosa which is directly attached to the bone. Following an inflammatory insult such as an upper respiratory tract infection or allergic rhinitis, a purulent form of sinusitis can develop. Occasionally secretions can be retained in the sinus due to altered ciliary function or obstruction of the opening (ostea) that drains the sinus. Incomplete drainage makes the sinus prone to infection typically with Haemophilus influenza, Streptococcus pneumoniae, Moraxella catarrhalis, Veillonella, Peptococcus, Corynebacterium acnes and certain species of fungi.

When initial treatment such as antibiotics, intranasal steroid sprays and decongestants are ineffective, it may become necessary to perform surgical drainage of the infected sinus. Surgical therapy often involves debridement of the ostea to remove anatomic obstructions and removal of parts of the mucosa. Occasionally a stent (a cylindrical tube which physically holds the lumen of the ostea open) is left in the osta to ensure drainage is maintained even in the presence of postoperative swelling. ENT stents, typically made of stainless steel or plastic, remain in place for several days or several weeks before being removed.

Representative examples of ENT stents that can benefit from being coated with or having incorporated therein a fibrosis-inhibiting agent include lacrimal duct stents, Eustachian tube stents, nasal stents, and sinus stents.

In one aspect, the present invention provides for the combination of a lacrimal duct stent and a fibrosis-inhibiting agent or a composition comprising a fibrosis-inhibiting agent.

In another aspect, the present invention provides for the combination of a Eustachian tube stent and a fibrosis-inhibiting agent or a composition comprising a fibrosis-inhibiting agent.

In yet another aspect, the present invention provides for the combination of a sinus stent and a fibrosis-inhibiting agent or a composition comprising a fibrosis-inhibiting agent.

In yet another aspect, the present invention provides for the combination of a nasal stent and a fibrosis-inhibiting agent or a composition comprising a fibrosis-inhibiting agent.

The ENT stent may be a choanal atresia stent composed of two long hollow tubes that are bridged by a flexible transverse tube. See, e.g., U.S. Pat. No. 6,606,995. The ENT stent may be an expandable nasal stent for postoperative nasal packing composed of a highly porous, pliable and absorbent foam material capable of expanding outwardly, which has a nonadherent surface. See, e.g., U.S. Pat. No. 5,336,163. The ENT stent may be a nasal stent composed of a deformable cylinder with a breathing passageway that has a smooth outer non-absorbent surface used for packing the nasal cavity following surgery. See, e.g., U.S. Pat. No. 5,601,594. The ENT stent may be a ventilation tube composed of a flexible, plastic, tubular vent with a rectangular flexible flange which is used for the nasal sinuses following endoscopic antrostomy. See, e.g., U.S. Pat. No. 5,246,455. The ENT stent may be a ventilating ear tube composed of a shaft and an extended tab which is used for equalizing the pressure between the middle ear and outer ear. See, e.g., U.S. Pat. No. 6,042,574. The ENT stent may be a middle ear vent tube composed of a non-compressible, tubular base and an eccentric flange. See, e.g., U.S. Pat. No. 5,047,053.

ENT stents, which may be combined with one or more agents according to the present invention, include commercially available products such as Genzyme Corporation (Ridgefield, N.J.) SEPRAGEL Sinus Stents and MEROGEL Nasal Dressing and Sinus Stents from Medtronic Xomed Surgical Products, Inc. (Jacksonville, Fla.).

In one aspect, the present invention provides ENT stents that include an anti-scarring agent or a composition that includes an anti-scarring agent. Numerous polymeric and non-polymeric delivery systems for use in ENT stents have been described above. Methods for incorporating fibrosis-inhibiting compositions onto or into the ENT stents include: (a) directly affixing to the stent a fibrosis-inhibiting composition (e.g., by either a spraying process or dipping process as described above, with or without a carrier), (b) directly incorporating into the stent a fibrosis-inhibiting composition (e.g., by either a spraying process or dipping process as described above, with or without a carrier), (c) by coating the stent with a substance such as a hydrogel which will in turn absorb the fibrosis-inhibiting composition, (d) by interweaving fibrosis-inhibiting composition coated thread (or the polymer itself formed into a thread) into the stent structure, (e) by inserting the stent into a sleeve or mesh which is comprised of or coated with a fibrosis-inhibiting composition, (f) constructing the stent itself or a portion of the stent with a fibrosis-inhibiting composition, or (g) by covalently binding the fibrosis-inhibiting agent directly to the stent surface or to a linker (small molecule or polymer) that is coated or attached to the stent surface. For these devices, the coating process can be performed in such a manner as to (a) coat the external surface of the specific stent, (b) coat the internal (luminal) surface of the stent, or (c) coat all or parts of both the internal and external surfaces of the device.

In addition to coating the device with the fibrosis-inhibiting composition, the fibrosis-inhibiting agent can be mixed with the materials that are used to make the device such that the fibrosis-inhibiting agent is incorporated into the final device.

According to the present invention, any fibrosis-inhibiting agent described above can be utilized in the practice of this embodiment. Within one embodiment of the invention, ENT stents may be adapted to release an agent that inhibits one or more of the four general components of the process of fibrosis (or scarring), including: formation of new blood vessels (angiogenesis), migration and proliferation of connective tissue cells (such as fibroblasts or smooth muscle cells), deposition of extracellular matrix (ECM), and remodeling (maturation and organization of the fibrous tissue). By inhibiting one or more of the components of fibrosis (or scarring), the overgrowth of granulation tissue may be inhibited or reduced.

As ENT stents are made in a variety of configurations and sizes, the exact dose administered will vary with device size, surface area and design. However, certain principles can be applied in the application of this art. Drug dose can be calculated as a function of dose per unit area (of the portion of the device being coated), total dose administered, and appropriate surface concentrations of active drug can be determined. Drugs are to be used at concentrations that range from several times more than to 10%, 5%, or even less than 1% of the concentration typically used in a single chemotherapeutic systemic dose application. Preferably, the drug is released in effective concentrations for a period ranging from 1-90 days.

Several examples of fibrosis-inhibiting agents for use in ENT stents include the following: Cell Cycle Inhibitors including (A) anthracyclines (e.g., doxorubicin and mitoxantrone), (B) taxanes (e.g., paclitaxel, TAXOTERE and docetaxel), and (C) podophyllotoxins (e.g., etoposide); (D) immunomodulators (e.g., sirolimus, everolimus, tacrolimus); (E) heat shock protein 90 antagonists (e.g., geldanamycin); (F) HMGCoA reductase inhibitors (e.g., simvastatin); (G) inosine monophosphate dehydrogenase inhibitors (e.g., mycophenolic acid, 1-alpha-25 dihydroxy vitamin D₃); (H)NF kappa B inhibitors (e.g., Bay 11-7082); (I) antimycotic agents (e.g., sulconizole), (J) p38 MAP kinase inhibitors (e.g., SB202190), and (K) and anti-angiogenesis agents (e.g., halofuginone bromide), as well as analogues and derivatives of the aforementioned.

Regardless of the method of application of the drug to the ENT stent, the exemplary anti-fibrosing agents, used alone or in combination, should be administered under the following dosing guidelines. The total amount (dose) of anti-scarring agent in or on the device may be in the range of about 0.01 μg-10 μg, or 10 μg-10 mg, or 10 mg-250 mg, or 250 mg-1000 mg, or 1000 mg-2500 mg. The dose (amount) of anti-scarring agent per unit area of device surface to which the agent is applied may be in the range of about 0.01 μg/mm²-1 μg/mm², or 1 μg/mm²-10 μg/mm², or 10 μg/mm²-250 μg/mm², 250 μg/mm²-1000 μg/mm², or 1000 μg/mm²-2500 μg/mm².

Provided below are exemplary dosage ranges for various anti-scarring agents that can be used in conjunction with ENT stent devices in accordance with the invention. A) Cell cycle inhibitors including doxorubicin and mitoxantrone. Doxorubicin analogues and derivatives thereof: total dose not to exceed 25 mg (range of 0.1 μg to 25 mg); preferred 1 μg to 5 mg. The dose per unit area of 0.01 μg-100 μg per mm²; preferred dose of 0.1 μg/mm²-10 μg/mm². Minimum concentration of 10⁻⁸-10⁻⁴ M of doxorubicin is to be maintained on the device surface. Mitoxantrone and analogues and derivatives thereof: total dose not to exceed 5 mg (range of 0.01 μg to 5 mg); preferred 0.1 μg to 1 mg. The dose per unit area of the device of 0.01 μg-20 μg per mm²; preferred dose of 0.05 μg/mm²-3 μg/mm². Minimum concentration of 10⁻⁸-10⁻⁴ M of mitoxantrone is to be maintained on the device surface. B) Cell cycle inhibitors including Paclitaxel and analogues and derivatives (e.g., docetaxel) thereof: total dose not to exceed 10 mg (range of 0.1 μg to 10 mg); preferred 1 μg to 3 mg. The dose per unit area of the device of 0.1 μg-10 μg per mm²; preferred dose of 0.25 μg/mm²-5 μg/mm². Minimum concentration of 10⁻⁸-10⁻⁴ M of paclitaxel is to be maintained on the device surface. (C) Cell cycle inhibitors such as podophyllotoxins (e.g., etoposide): total dose not to exceed 10 mg (range of 0.1 μg to 10 mg); preferred 1 μg to 3 mg. The dose per unit area of the device of 0.1 μg-10 μg per mm²; preferred dose of 0.25 μg/mm²-5 μg/mm². Minimum concentration of 10⁻⁸-10⁻⁴ M of etoposide is to be maintained on the device surface. (D) Immunomodulators including sirolimus and everolimus. Sirolimus (i.e., rapamycin, RAPAMUNE): Total dose not to exceed 10 mg (range of 0.1 μg to 10 mg); preferred 10 μg to 1 mg. The dose per unit area of 0.1 μg-100 μg per mm²; preferred dose of 0.5 μg/mm²-10 μg/mm². Minimum concentration of 10⁻⁸-10⁻⁴ M is to be maintained on the device surface. Everolimus and derivatives and analogues thereof: Total dose should not exceed 10 mg (range of 0.1 μg to 10 mg); preferred 10 μg to 1 mg. The dose per unit area of 0.1 μg-100 μg per mm² of surface area; preferred dose of 0.3 μg/mm²-10 μg/mm². Minimum concentration of 10⁻⁸-10⁻⁴ M of everolimus is to be maintained on the device surface. (E) Heat shock protein 90 antagonists (e.g., geldanamycin) and analogues and derivatives thereof: total dose not to exceed 20 mg (range of 0.1 μg to 20 mg); preferred 1 μg to 5 mg. The dose per unit area of the device of 0.1 μg-10 μg per mm²; preferred dose of 0.25 μg/mm²-5 μg/mm². Minimum concentration of 10⁻⁸-10⁻⁴ M of geldanamycin is to be maintained on the device surface. (F) HMGCoA reductase inhibitors (e.g., simvastatin) and analogues and derivatives thereof: total dose not to exceed 2000 mg (range of 10.0 μg to 2000 mg); preferred 10 μg to 300 mg. The dose per unit area of the device of 1.0 μg-1000 μg per mm²; preferred dose of 2.5 μg/mm²-500 μg/mm². Minimum concentration of 10⁻⁸-10⁻³ M of simvastatin is to be maintained on the device surface. (G) Inosine monophosphate dehydrogenase inhibitors (e.g., mycophenolic acid, 1-alpha-25 dihydroxy vitamin D₃) and analogues and derivatives thereof: total dose not to exceed 2000 mg (range of 10.0 μg to 2000 mg); preferred 10 μg to 300 mg. The dose per unit area of the device of 1.0 μg-1000 μg per mm²; preferred dose of 2.5 g/mm²-500 μg/mm². Minimum concentration of 10⁻⁸-10⁻³ M of mycophenolic acid is to be maintained on the device surface. (H)NF kappa B inhibitors (e.g., Bay 11-7082) and analogues and derivatives thereof: total dose not to exceed 200 mg (range of 1.0 μg to 200 mg); preferred 1 μg to 50 mg. The dose per unit area of the device of 1.0 μg-100 μg per mm²; preferred dose of 2.5 μg/mm²-50 μg/mm². Minimum concentration of 10⁻⁸-10⁻⁴ M of Bay 11-7082 is to be maintained on the device surface. (I) Antimycotic agents (e.g., sulconizole) and analogues and derivatives thereof: total dose not to exceed 2000 mg (range of 10.0 μg to 2000 mg); preferred 10 μg to 300 mg. The dose per unit area of the device of 1.0 μg-1000 μg per mm²; preferred dose of 2.5 μg/mm²-500 μg/mm². Minimum concentration of 10⁻⁸-10⁻³ M of sulconizole is to be maintained on the device surface. (J) p38 MAP kinase inhibitors (e.g., SB202190) and analogues and derivatives thereof: total dose not to exceed 2000 mg (range of 10.0 μg to 2000 mg); preferred 10 μg to 300 mg. The dose per unit area of the device of 1.0 μg-1000 μg per mm²; preferred dose of 2.5 μg/mm²-500 μg/mm². Minimum concentration of 10⁻⁸-10⁻³ M of SB202190 is to be maintained on the device surface. (K) Anti-angiogenic agents (e.g., halofuginone bromide) and analogues and derivatives thereof: total dose not to exceed 10 mg (range of 0.1 μg to 10 mg); preferred 1 μg to 3 mg. The dose per unit area of the device of 0.1 μg-10 μg per mm²; preferred dose of 0.25 μg/mm²-5 μg/mm². Minimum concentration of 10⁻⁸-10⁻⁴ M of halofuginone bromide is to be maintained on the device surface.

In addition to those described above (e.g., sirolimus, everolimus, and tacrolimus), several other examples of immunomodulators and appropriate dosages ranges for use with ENT stent devices include the following: (A) Biolimus and derivatives and analogues thereof: Total dose should not exceed 10 mg (range of 0.1 μg to 10 mg); preferred 10 μg to 1 mg. The dose per unit area of 0.1 μg-100 μg per mm² of surface area; preferred dose of 0.3 μg/mm²-10 μg/mm². Minimum concentration of 10⁻⁸-10⁻⁴ M of everolimus is to be maintained on the device surface. (B) Tresperimus and derivatives and analogues thereof: Total dose should not exceed 10 mg (range of 0.1 μg to 10 mg); preferred 10 μg to 1 mg. The dose per unit area of 0.1 μg-100 μg per mm² of surface area; preferred dose of 0.3 μg/mm²-10 μg/mm². Minimum concentration of 10⁻⁸-10⁻⁴ M of tresperimus is to be maintained on the device surface. (C) Auranofin and derivatives and analogues thereof: Total dose should not exceed 10 mg (range of 0.1 μg to 10 mg); preferred 10 μg to 1 mg. The dose per unit area of 0.1 μg-100 μg per mm² of surface area; preferred dose of 0.3 μg/mm²-10 μg/mm². Minimum concentration of 10⁻⁸-10⁻⁴ M of auranofin is to be maintained on the device surface. (D) 27-0-Demethylrapamycin and derivatives and analogues thereof: Total dose should not exceed 10 mg (range of 0.1 μg to 10 mg); preferred 10 μg to 1 mg. The dose per unit area of 0.1 μg-100 μg per mm² of surface area; preferred dose of 0.3 μg/mm²-10 μg/mm². Minimum concentration of 10⁻⁸-10⁻⁴ M of 27-0-Demethylrapamycin is to be maintained on the device surface. (E) Gusperimus and derivatives and analogues thereof: Total dose should not exceed 10 mg (range of 0.1 μg to 10 mg); preferred 10 μg to 1 mg. The dose per unit area of 0.1 μg-100 μg per mm² of surface area; preferred dose of 0.3 μg/mm²-10 μg/mm². Minimum concentration of 10⁻⁸-10⁻⁴ M of gusperimus is to be maintained on the device surface. (F) Pimecrolimus and derivatives and analogues thereof: Total dose should not exceed 10 mg (range of 0.1 μg to 10 mg); preferred 10 μg to 1 mg. The dose per unit area of 0.1 μg-100 μg per mm² of surface area; preferred dose of 0.3 μg/mm²-10 μg/mm². Minimum concentration of 10⁻⁸-10⁻⁴ M of pimecrolimus is to be maintained on the device surface and (G) ABT-578 and analogues and derivatives thereof: Total dose should not exceed 10 mg (range of 0.1 μg to 10 mg); preferred 10 μg to 1 mg. The dose per unit area of 0.1 μg-100 μg per mm² of surface area; preferred dose of 0.3 μg/mm²-10 μg/mm². Minimum concentration of 10⁻⁸-10⁻⁴ M of ABT-578 is to be maintained on the device surface.

Ear Ventilation Tubes

In another aspect, the present invention provides for the combination of an anti-scarring agent and an ear ventilation tube (also referred to as a tympanostomy tube).

Acute otitis media is the most common bacterial infection, the most frequent indication for surgical therapy, the leading cause of hearing loss and a common cause of impaired language development in children. The cost of treating this condition in children under the age of five is estimated at $5 billion annually in the United States alone. In fact, 85% of all children will have at least one episode of otitis media and 600,000 will require surgical therapy annually. The prevalence of otitis media is increasing and for severe cases surgical therapy is more cost effective than conservative management.

Acute otitis media (bacterial infection of the middle ear) is characterized by Eustachian tube dysfunction leading to failure of the middle ear clearance mechanism. The most common causes of otitis media are Streptococcus pneumoniae (30%), Haemophilus influenza (20%), Branhamella catarrhalis (12%), Streptococcus pyogenes (3%), and Staphylococcus aureus (1.5%). The end result is the accumulation of bacteria, white blood cells and fluid which, in the absence of an ability to drain through the Eustachian tube, results in increased pressure in the middle ear. For many cases antibiotic therapy is sufficient treatment and the condition resolves. However, for a significant number of patients the condition becomes frequently recurrent or does not resolve completely. In recurrent otitis media or chronic otitis media with effusion, there is a continuous build-up of fluid and bacteria that creates a pressure gradient across the tympanic membrane causing pain and impaired hearing. Fenestration of the tympanic membrane (typically with placement of a tympanostomy tube) relieves the pressure gradient and facilitates drainage of the middle ear (through the outer ear instead of through the Eustachian tube—a form of “Eustachian tube bypass”).

Recurrent otitis media or otitis media with effusion may be treated with tympanostomy tubes or artificial Eustachian tubes/stents, such as described above. These ventilation tubes are indicated for chronic otitis media with effusion, recurrent acute otitis media, tympanic membrane atelectasis, and complications of acute otitis media in children. The excessive formation of granulation tissue around these devices can result in a decreased functioning of these devices. This can then result in a second procedure to either clear the obstruction or to insert a new device. The incorporation of a fibrosis-inhibiting agent into or onto the ventilation tubes may prevent the overgrowth of this granulation tissue.

Surgical placement of tympanostomy tubes is the most widely used treatment for chronic otitis media because, although not curative, it improves hearing (which in turn improves language development) and reduces the incidence of acute otitis media. Tympanostomy tube placement is one of the most common surgical procedures in the United States with 1.3 million surgical placements per year.

Representative examples of ear ventilation tubes that can benefit from being coated with or having incorporated therein a fibrosis-inhibiting agent include, without limitation, grommet-shaped tubes, T-tubes, tympanostomy tubes, drain tubes, tympanic tubes, otological tubes, myringotomy tubes, artificial Eustachian tubes, Eustachian tube prostheses, and Eustachian stents. Ear ventilation tubes have been made out of, e.g., polytetrafluoroethylene (e.g., TEFLON), silicone, nylon, polyethylene and other polymers, stainless steel, titanium, and gold plated steel.

In one aspect, the ear ventilation tube may be a tympanostomy tube that is used to provide an alternative conduit for ventilation of the middle ear cavity via the external ear canal. Typically, ventilation of the middle ear is performed by conducting a myringotomy, in which a slit or opening in the tympanic membrane is surgically made to alleviate a buildup or reduction of pressure in the middle ear cavity and to drain accumulated fluids. Tympanostomy tubes may be inserted into the surgical slit of the tympanic membrane to serve as a bypass for the normal Eustachian tube, which drains the middle ear cavity under normal conditions. For example, the tympanostomy tube may be an elongated uniform tubular member composed of pure titanium or titanium alloy that has a concavity inwardly spaced from one end that forms a flange. See, e.g., U.S. Pat. No. 5,645,584. The tympanostomy tube may be composed of a micro-pitted titanium exterior flangeless surface used to ventilate the middle ear. See, e.g., U.S. Pat. No. 4,971,076. The tympanostomy tube may be composed of a shaft with a tab that extends outwardly perpendicular from the bottom of the shaft. See, e.g., U.S. Pat. No. 6,042,574. The tympanostomy tube may be a permanent ear ventilation device composed of an elongated tubular base having a flange eccentrically connected made of a non-compressible material. See, e.g., U.S. Pat. No. 5,047,053. The tympanostomy tube may be composed of a cap-plug, central body and end cap, which together form a plurality of lumens within the tube. See, e.g., U.S. Pat. No. 5,851,199. The tympanostomy tube may be composed of a microporous resin cured to form a gas-permeable matrix containing a homogenous dispersion of silver particles capable of migrating to the surface of the tube sidewalls to provide antimicrobial activity. See, e.g., U.S. Pat. No. 6,361,526. The tympanostomy tube may be composed of tubular body and a rib structure that projects outwardly to define a channel spiraling around the tubular body. See, e.g., U.S. Pat. No. 5,775,336. The tympanostomy tube may be composed of an integral cutting tang extending from one of two flanges of a grommet for incising the tympanic membrane. See, e.g., U.S. Pat. Nos. 5,827,295 and 5,643,280. The tympanostomy tube may be composed of a tubular member having two opposed flanges in which the insertion of the tube is facilitated by a cutting edge on the flange which induces an incision of the tympanic membrane. See, e.g., U.S. Pat. Nos. 5,489,286; 5,466,239; 5,254,120 and 5,207,685. Other tympanostomy tubes are described in, e.g., U.S. Pat. Nos. 6,406,453; 5,178,623; 4,808,171 and 4,744,792.

In another aspect, the ear ventilation tube may be used to establish the normal function of the Eustachian tube and thus, attempt to resolve the stenosis that prevents its normal function. Fluid in the middle ear cavity normally secretes away from the tympanic membrane and thus, restoring the normal function of the Eustachian tube may provide optimal ventilation and drainage. For example, the ventilation tube may be an Eustachian stent composed of a hollow tubular body having a compressible core with two connected parallel arms and a radially-oriented flange, which is placed in the Eustachian tube to maintain patency. See, e.g., U.S. Pat. No. 6,589,286. The ventilation tube may be an Eustachian tube prosthesis composed of a flexible tube having a flange that extends radially for positioning within the Eustachian tube passageway. See, e.g., U.S. Pat. No. 4,015,607.

Tympanostomy tubes, which may be combined with one or more agents according to the present invention, include commercially available products. For example, Medtronic Xomed, Inc. (Jackonsville, Fla.) sells a variety of ear ventilation tubes, including Long-Term Ventilation Tubes and Grommet Style Ventilation Tubes, including ARMSTRONG Grommets, GOODE T-Grommets, VENTUR1 Style Ventilation Tubes, SHEEHY Type Collar Buttons, REUTER Bobbins, COHEN T-Grommets, and SOILEAU TYTAN Titanium Tubes. Micromedics, Inc. (Eagan, Minn.) also sells a variety of ear ventilation tubes, including BAXTER Bevel Buttons, TINY TOUMA, SPOONER, TOUMA T-Tubes, SHOEHORN Bobbins, SHAH, and SILVERSTEIN MICROWICK Eustachian Tubes. Gyrus ENT LLC (Bartlett, Tenn.) also sells a variety of ear ventilation tubes, including ULTRASIL Ventilation Tubes, RICHARDS COLLAR Bobbins, BALDWIN BUTTERFLY Ventilation Tubes and PAPARELLA 2000 Tubes.

In one aspect, the present invention provides ear ventilation tube devices that include an anti-scarring agent or a composition that includes an anti-scarring agent. Numerous polymeric and non-polymeric delivery systems for use in ear ventilation tubes have been described above. These compositions can further include one or more fibrosis-inhibiting agents such that the overgrowth of granulation tissue is inhibited or reduced.

Numerous polymeric and non-polymeric delivery systems for use in ear ventilation tubes have been described above. Methods for incorporating the fibrosis-inhibiting agent or a composition comprising the fibrosis-inhibiting agent into or onto the device includes: (a) directly affixing to the device a fibrosis-inhibiting composition (e.g., by either a spraying process or dipping process as described above, with or without a carrier), (b) directly incorporating into the device a fibrosis-inhibiting composition (e.g., by either a spraying process or dipping process as described above, with or without a carrier, (c) by coating the device with a substance such as a hydrogel which will in turn absorb the fibrosis-inhibiting composition, (d) by interweaving fibrosis-inhibiting composition coated thread (or the polymer itself formed into a thread) into the device structure, (e) constructing the device itself or a portion of the device with a fibrosis-inhibiting composition, or (f) by covalently binding the fibrosis-inhibiting agent directly to the device surface or to a linker (small molecule or polymer) that is coated or attached to the device surface. The coatings can be applied to different portions of the device. For example, the coating can be (a) a coating applied to the external surface of the ear ventilation tube; (b) a coating applied to the internal (luminal) surface of the ear ventilation tube; or (c) a coating applied to all or parts of both surfaces.

In addition to coating the device with the fibrosis-inhibiting composition, the fibrosis-inhibiting agent can be mixed with the materials that are used to make the device such that the fibrosis-inhibiting agent is incorporated into the final device.

In addition to incorporation of a fibrosis-inhibiting agent into or onto the device, another biologically active agent can be incorporated into or onto the device, for example an anti-inflammatory (e.g., dexamethazone or aspirin) and/or an antibiotic (e.g., amoxicillin, trimethoprim-sulfamethoxazole, azithromycin, clarithromycin, amoxicillin-clavulanate, cefprozil, cefuroxime, cefpodoxime, or cefdinir).

According to the present invention, any fibrosis-inhibiting agent described above can be utilized in the practice of this embodiment. Within one embodiment of the invention, ear ventilation tubes may be adapted to release an agent that inhibits one or more of the four general components of the process of fibrosis (or scarring), including: formation of new blood vessels (angiogenesis), migration and proliferation of connective tissue cells (such as fibroblasts or smooth muscle cells), deposition of extracellular matrix (ECM), and remodeling (maturation and organization of the fibrous tissue). By inhibiting one or more of the components of fibrosis (or scarring), the overgrowth of granulation tissue may be inhibited or reduced.

As ear ventilation tube devices are made in a variety of configurations and sizes, the exact dose administered will vary with device size, surface area and design. However, certain principles can be applied in the application of this art. Drug dose can be calculated as a function of dose per unit area (of the portion of the device being coated), total dose administered, and appropriate surface concentrations of active drug can be determined. Drugs are to be used at concentrations that range from several times more than to 10%, 5%, or even less than 1% of the concentration typically used in a single chemotherapeutic systemic dose application. Preferably, the drug is released in effective concentrations for a period ranging from 1-90 days.

Several examples of fibrosis-inhibiting agents for use in ear ventilation tubes include the following: cell cycle inhibitors including (A) anthracyclines (e.g., doxorubicin and mitoxantrone), (B) taxanes (e.g., paclitaxel, TAXOTERE and docetaxel), and (C) podophyllotoxins (e.g., etoposide); (D) immunomodulators (e.g., sirolimus, everolimus, tacrolimus); (E) heat shock protein 90 antagonists (e.g., geldanamycin); (F) HMGCoA reductase inhibitors (e.g., simvastatin); (G) inosine monophosphate dehydrogenase inhibitors (e.g., mycophenolic acid, 1-alpha-25 dihydroxy vitamin D₃); (H)NF kappa B inhibitors (e.g., Bay 11-7082); (I) antimycotic agents (e.g., sulconizole), (J) p38 MAP kinase inhibitors (e.g., SB202190), and (K) and anti-angiogenesis agents (e.g., halofuginone bromide), as well as analogues and derivatives of the aforementioned.

Regardless of the method of application of the drug to the ear ventilation tube device, the exemplary anti-fibrosing agents, used alone or in combination, should be administered under the following dosing guidelines. The total amount (dose) of anti-scarring agent in or on the device may be in the range of about 0.01 μg-10 μg, or 10 μg-10 mg, or 10 mg-250 mg, or 250 mg-1000 mg, or 1000 mg-2500 mg. The dose (amount) of anti-scarring agent per unit area of device surface to which the agent is applied may be in the range of about 0.01 μg/mm²-1 μg/mm², or 1 μg/mm²-10 μg/mm², or 10 μg/mm²-250 μg/mm², 250 μg/mm²-1000 μg/mm², or 1000 μg/mm²-2500 μg/mm².

Provided below are exemplary dosage ranges for various anti-scarring agents that can be used in conjunction with ear ventilation tube devices in accordance with the invention. A) Cell cycle inhibitors including doxorubicin and mitoxantrone. Doxorubicin analogues and derivatives thereof: total dose not to exceed 25 mg (range of 0.1 μg to 25 mg); preferred 1 μg to 5 mg. The dose per unit area of 0.01 μg-100 μg per mm²; preferred dose of 0.1 μg/mm²-10 μg/mm². Minimum concentration of 10⁻⁸-10⁻⁴ M of doxorubicin is to be maintained on the device surface. Mitoxantrone and analogues and derivatives thereof: total dose not to exceed 5 mg (range of 0.01 μg to 5 mg); preferred 0.1 μg to 1 mg. The dose per unit area of the device of 0.01 μg-20 μg per mm²; preferred dose of 0.05 μg/mm²-3 μg/mm². Minimum concentration of 10⁻⁸-10⁻⁴ M of mitoxantrone is to be maintained on the device surface. B) Cell cycle inhibitors including Paclitaxel and analogues and derivatives (e.g., docetaxel) thereof: total dose not to exceed 10 mg (range of 0.1 μg to 10 mg); preferred 1 μg to 3 mg. The dose per unit area of the device of 0.1 μg-10 μg per mm²; preferred dose of 0.25 μg/mm²-5 μg/mm². Minimum concentration of 10⁻⁸-10⁻⁴ M of paclitaxel is to be maintained on the device surface. (C) Cell cycle inhibitors such as podophyllotoxins (e.g., etoposide): total dose not to exceed 10 mg (range of 0.1 μg to 10 mg); preferred 1 μg to 3 mg. The dose per unit area of the device of 0.1 μg-10 μg per mm²; preferred dose of 0.25 μg/mm²-5 μg/mm². Minimum concentration of 10⁻⁸-10⁻⁴ M of etoposide is to be maintained on the device surface. (D) Immunomodulators including sirolimus and everolimus. Sirolimus (i.e., rapamycin, RAPAMUNE): Total dose not to exceed 10 mg (range of 0.1 μg to 10 mg); preferred 10 μg to 1 mg. The dose per unit area of 0.1 μg-100 μg per mm²; preferred dose of 0.5 μg/mm²-10 μg/mm². Minimum concentration of 10⁻⁸-10⁴ M is to be maintained on the device surface. Everolimus and derivatives and analogues thereof: Total dose should not exceed 10 mg (range of 0.1 μg to 10 mg); preferred 10 μg to 1 mg. The dose per unit area of 0.1 μg-100 μg per mm² of surface area; preferred dose of 0.3 μg/mm²-10 μg/mm². Minimum concentration of 10⁻⁸-10⁴ M of everolimus is to be maintained on the device surface. (E) Heat shock protein 90 antagonists (e.g., geldanamycin) and analogues and derivatives thereof: total dose not to exceed 20 mg (range of 0.1 μg to 20 mg); preferred 1 μg to 5 mg. The dose per unit area of the device of 0.1 μg-10 μg per mm²; preferred dose of 0.25 μg/mm²-5 μg/mm². Minimum concentration of 10⁻⁸-10⁻⁴ M of geldanamycin is to be maintained on the device surface. (F) HMGCoA reductase inhibitors (e.g., simvastatin) and analogues and derivatives thereof: total dose not to exceed 2000 mg (range of 10.0 μg to 2000 mg); preferred 10 μg to 300 mg. The dose per unit area of the device of 1.0 μg-1000 μg per mm²; preferred dose of 2.5 μg/mm²-500 μg/mm². Minimum concentration of 10⁻⁸-10⁻³ M of simvastatin is to be maintained on the device surface. (G) Inosine monophosphate dehydrogenase inhibitors (e.g., mycophenolic acid, 1-alpha-25 dihydroxy vitamin D₃) and analogues and derivatives thereof: total dose not to exceed 2000 mg (range of 10.0 μg to 2000 mg); preferred 10 μg to 300 mg. The dose per unit area of the device of 1.0 μg-1000 μg per mm²; preferred dose of 2.5 μg/mm²-500 μg/mm². Minimum concentration of 10⁻⁸-10⁻³ M of mycophenolic acid is to be maintained on the device surface. (H)NF kappa B inhibitors (e.g., Bay 11-7082) and analogues and derivatives thereof: total dose not to exceed 200 mg (range of 1.0 μg to 200 mg); preferred 1 μg to 50 mg. The dose per unit area of the device of 1.0 μg-100 μg per mm²; preferred dose of 2.5 g/mm²-50 μg/mm². Minimum concentration of 10⁻⁸-10⁻⁴ M of Bay 11-7082 is to be maintained on the device surface. (I) Antimycotic agents (e.g., sulconizole) and analogues and derivatives thereof: total dose not to exceed 2000 mg (range of 10.0 μg to 2000 mg); preferred 10 μg to 300 mg. The dose per unit area of the device of 1.0 μg-1000 μg per mm²; preferred dose of 2.5 μg/mm²-500 μg/mm². Minimum concentration of 10⁻⁸-10⁻³ M of sulconizole is to be maintained on the device surface. (J) p38 MAP kinase inhibitors (e.g., SB202190) and analogues and derivatives thereof: total dose not to exceed 2000 mg (range of 10.0 μg to 2000 mg); preferred 10 μg to 300 mg. The dose per unit area of the device of 1.0 μg-1000 μg per mm²; preferred dose of 2.5 μg/mm²-500 μg/mm². Minimum concentration of 10⁻⁸-10⁻³ M of SB202190 is to be maintained on the device surface. (K) Anti-angiogenic agents (e.g., halofuginone bromide) and analogues and derivatives thereof: total dose not to exceed 10 mg (range of 0.01 μg to 10 mg); preferred 1 μg to 3 mg. The dose per unit area of the device of 0.1 μg-10 μg per mm²; preferred dose of 0.25 μg/mm²-5 μg/mm². Minimum concentration of 10⁻⁸-10⁻⁴ M of halofuginone bromide is to be maintained on the device surface.

In addition to those described above (e.g., sirolimus, everolimus, and tacrolimus), several other examples of immunomodulators and appropriate dosages ranges for use with ear ventilation devices include the following: (A) Biolimus and derivatives and analogues thereof: Total dose should not exceed 10 mg (range of 0.1 μg to 10 mg); preferred 10 μg to 1 mg. The dose per unit area of 0.1 μg-100 μg per mm² of surface area; preferred dose of 0.3 μg/mm²-10 μg/mm². Minimum concentration of 10⁻⁸-10⁻⁴ M of everolimus is to be maintained on the device surface. (B) Tresperimus and derivatives and analogues thereof: Total dose should not exceed 10 mg (range of 0.1 μg to 10 mg); preferred 10 μg to 1 mg. The dose per unit area of 0.1 μg-100 μg per mm² of surface area; preferred dose of 0.3 μg/mm²-10 μg/mm². Minimum concentration of 10⁻⁸-10⁻⁴ M of tresperimus is to be maintained on the device surface. (C) Auranofin and derivatives and analogues thereof: Total dose should not exceed 10 mg (range of 0.1 μg to 10 mg); preferred 10 μg to 1 mg. The dose per unit area of 0.1 μg-100 μg per mm² of surface area; preferred dose of 0.3 μg/mm²-10 μg/mm². Minimum concentration of 10⁻⁸-10⁻⁴ M of auranofin is to be maintained on the device surface. (D) 27-0-Demethylrapamycin and derivatives and analogues thereof: Total dose should not exceed 10 mg (range of 0.1 μg to 10 mg); preferred 10 μg to 1 mg. The dose per unit area of 0.1 μg-100 μg per mm² of surface area; preferred dose of 0.3 μg/mm²-10 μg/mm². Minimum concentration of 10⁻⁸-10⁻⁴ M of 27-0-Demethylrapamycin is to be maintained on the device surface. (E) Gusperimus and derivatives and analogues thereof: Total dose should not exceed 10 mg (range of 0.1 μg to 10 mg); preferred 10 μg to 1 mg. The dose per unit area of 0.1 μg-100 μg per mm² of surface area; preferred dose of 0.3 μg/mm²-10 μg/mm². Minimum concentration of 10⁻⁸-10⁻⁴ M of gusperimus is to be maintained on the device surface. (F) Pimecrolimus and derivatives and analogues thereof: Total dose should not exceed 10 mg (range of 0.1 μg to 10 mg); preferred 10 μg to 1 mg. The dose per unit area of 0.1 μg-100 μg per mm² of surface area; preferred dose of 0.3 μg/mm²-10 μg/mm². Minimum concentration of 10⁻⁸-10⁻⁴ M of pimecrolimus is to be maintained on the device surface and (G) ABT-578 and analogues and derivatives thereof: Total dose should not exceed 10 mg (range of 0.1 μg to 10 mg); preferred 10 μg to 1 mg. The dose per unit area of 0.1 μg-100 μg per mm² of surface area; preferred dose of 0.3 μg/mm²-10 μg/mm². Minimum concentration of 10⁻⁸-10⁻⁴ M of ABT-578 is to be maintained on the device surface.

In addition to those described above (e.g., paclitaxel, TAXOTERE, and docetaxel), several other examples of anti-microtubule agents and appropriate dosages ranges for use with ear ventilation devices include vinca alkaloids such as vinblastine and vincristine sulfate and analogues and derivatives thereof: total dose not to exceed 10 mg (range of 0.1 μg to 10 mg); preferred 1 μg to 3 mg. Dose per unit area of the device of 0.1 μg-10 μg per mm²; preferred dose of 0.25 μg/mm²-5 μg/mm². Minimum concentration of 10⁻⁸-10⁻⁴ M of drug is to be maintained on the device surface.

Intraocular Implants

In another aspect, the present invention provides for the combination of an anti-scarring agent and an intraocular implant.

In one embodiment, the intraocular implant is an intraocular lens device for the prevention of lens (e.g., anterior or posterior lens) opacification. Eyesight deficiencies that may be treated with intraocular lenses include, without limitation, cataracts, myopia, hyperopia, astigmatism and other eye diseases. Intraocular lenses are most commonly used to replace the natural crystalline lens which is removed during cataract surgery. A cataract results from a change in the transparency of the normal crystalline lens in the eye. When the lens becomes opaque from calcification (e.g., yellow and/or cloudy), the light cannot enter the eye properly and vision is impaired.

Implantation of intraocular lenses into the eye is a standard technique to restore useful vision in diseased or damaged eyes. The number of intraocular lenses implanted in the United States has grown exponentially over the last decade. Currently, over 1 million intraocular lenses are implanted annually, with the vast majority (90%) being placed in the posterior chamber of the eye. The intent of intraocular lenses is to replace the natural crystalline lens (i.e., aphakic eye) or to supplement and correct refractive errors (i.e., phakic eye, natural crystalline lens is not removed).

Implanted intraocular lenses may develop complications caused by mechanical trauma, inflammation, infection or optical problems. Mechanical and inflammatory injury may lead to reduced vision, chronic pain, secondary cataracts, corneal decompensation, cystoid macular edema, hyphema, uveitis or glaucoma. One common problem that occurs with cataract extraction is opacification which results from the tissue's reaction to the surgical procedure or to the artificial lens. Opacification leads to clouding of the intraocular lens, thus reducing the long-term benefits. Opacification typically results when proliferation and migration of epithelial cells occur along the posterior capsule behind the intraocular lens. Subsequent surgery may be required to correct this reaction; however, it involves a complex technical process and may lead to further serious, sight-threatening complications. Therefore, coating or incorporating the intraocular lens with a fibrosis-inhibiting agent may reduce these complications.

Representative examples of intraocular lenses that can benefit from being coated with or having incorporated therein a fibrosis-inhibiting agent include, without limitation, polymethylmethacrylate (PMMA) intraocular lenses, silicone intraocular lenses, achromatic lenses, pseudophakos, phakic lenses, aphakic lenses, multi-focal intraocular lenses, hydrophilic and hydrophobic acrylic intraocular lenses, intraocular implants, optic lenses and rigid gas permeable (RGP) lenses.

In one aspect, intraocular lenses may be foldable or rigid. The foldable lenses may be inserted in a small incision site using a tiny tube whereas the hard lenses are inserted through a larger incision site. Foldable lenses may be composed of silicone, acrylic or hydrogel whereas rigid lenses may be composed of hard polymeric compositions (PMMA).

In one aspect, the intraocular lens may be used as an implant for the treatment of cataracts, where the natural crystalline lens of the eye has been removed (i.e., aphakic lens). For example, the intraocular lens may be composed of two lenses having distinct refractive indices and distinct optical powers being joined together as an achromatic lens that may be connected within a posterior or anterior chamber of the eye. See, e.g., U.S. Pat. No. 5,201,762. The intraocular lens may be secured in the posterior chamber by a system of posts that protrude through the iris attached to retaining rings. See, e.g., U.S. Pat. No. 4,053,953. The intraocular lens may be hard with a shape memory which is capable of deforming for insertion into the eye but will harden at normal body temperature. See, e.g., U.S. Pat. No. 4,946,470. The intraocular lens may be coated with proteins, polypeptides, polyamino acids, polyamines or carbohydrates bound to the surface of the implant. See, e.g., U.S. Pat. Nos. 6,454,802 and 6,106,554. Other examples of aphakic intraocular lenses are described in, e.g., U.S. Pat. Nos. 6,599,317; 6,585,768; 6,558,419; 6,533,813; 6,210,438; 5,266,074; 4,753,654; 4,718,904 and 4,704,123.

In another aspect, the intraocular lens may be used as a corrective implant for vision impairment, where the natural crystalline lens of the eye has not been removed (i.e., phakic lens). For example, the intraocular lens may be a narrow profile, glare reducing, phakic anterior chamber lens that may be composed of an optic zone and a transition zone that has a curvature shaped to minimize direct glare. See, e.g., U.S. Pat. No. 6,596,025. The intraocular lens may be a self-centering phakic lens inserted in the posterior chamber lens in which arms (i.e., haptic bodies) extend outwardly and protrude into the pupil such that the iris provides centering force to keep lens in place. See, e.g., U.S. Pat. No. 6,015,435. The intraocular lens may be composed of a circumferential edge and two haptics extending from the edge to a transverse member which is substantially straight or bowed inward toward the lens. See, e.g., U.S. Pat. No. 6,241,777. Other examples of phakic intraocular lenses are described in, e.g., U.S. Pat. Nos. 6,228,115; 5,480,428 and 5,222,981.

In another aspect, the intraocular lens may be a multi-focal lens capable of variable accommodation to enable the user to look through different portions of the lens to achieve different levels of focusing power. For example, the intraocular lens may be a variable focus lens composed of two lens portions with an optical zone between the lenses which may contain a fluid reservoir and channel containing charged solution. See, e.g., U.S. Pat. No. 5,443,506.

In another aspect, intraocular lenses may be deformable such that the lens may be folded for insertion through a tunnel incision. For example, the intraocular lens may be composed of a lens with fixation members for retaining the lens in the eye which may be configured for folding or rolling from a normal optical condition into an insertion condition to permit the lens to be passed through an incision into the eye. See, e.g., U.S. Pat. No. 5,476,513. The intraocular lens may be composed of a resilient, deformable silicone based optic with a fixation means coupled to the optic for retaining the optic in the eye. See, e.g., U.S. Pat. No. 5,201,763. The intraocular lens may be composed of a copolymer of three constituents which may be deformable from its original shape. See, e.g., U.S. Pat. No. 5,359,021. The intraocular lens may be composed of a transparent, flexible membrane with an interior sac and an attached bladder, in which optical fluid medium is shunted from the optical element to the bladder to aid in its deformity during insertion. See, e.g., U.S. Pat. No. 6,048,364. The intraocular lens may be a biocomposite composed of an optic portion made of high water content hydrogel capable of being folded and a haptic portion of low water content hydrogel having strength and rigidity. See, e.g., U.S. Pat. No. 5,211,662. Other deformable intraocular lenses are described in, e.g., U.S. Pat. Nos. 6,267,784; 5,507,806 and U.S. Patent Application Publication No. 2003/0114928A1.

Other related devices and/or compositions (e.g., insertion devices) that may be used in conjunction with intraocular lenses are described in, e.g., U.S. Pat. Nos. 6,629,979; 6,187,042; 6,113,633; 4,740,282 and U.S. Patent Application Publication Nos. 2003/0212409A1 and 2003/0187455A1.

Intraocular lenses, which may be combined with one or more agents according to the present invention, include commercially available products. For example, Alcon Laboratories, Inc. (Fort Worth, Tex.) sells the foldable ACRYSOF Intraocular Lens. Bausch & Lomb Surgical, Inc. (San Dimas, Calif.) sells the foldable SOFLEX SE Intraocular Lens. Advanced Medical Optics, Inc (Santa Ana, Calif.) sells the CLARIFLEX Foldable Intraocular Lens, SENSAR Acrylic Intraocular Lens, and PHACOFLEX II SI40NB and SI30NB.

The intraocular implant may comprise the fibrosis-inhibiting agent or a composition that includes the fibrosis-inhibiting agent directly. Alternatively, or in addition, the agent may be coated, absorbed into, or bound onto the lens surface (e.g., to the haptics), or may be released from a hole (pore) or cavity outside the optical part of the lens surface.

The intraocular implants of the invention may be used in various surgical procedures. For example, the intraocular implant may be used in conjunction with a transplant for the cornea. Synthetic corneas can be used in patients loosing vision due to a degenarative cornea. Implanted synthetic corneas can restore patient vision, however, they often induce a fibrous foreign body response that limits their use. The intraocular implant of the present invention can prevent the foreign body response to the synthetic cornea and extend the cornea longevity. In another example, the synthetic cornea itself is coated with the agents of the invention, thus minimizing tissue reaction to corneal implantation.

In another aspect, the intraocular lens may be used in conjunction with treatment of secondary cataract after extracapsular cataract extraction.

As described above, the present invention provides intraocular lenses and other implants that include an anti-scarring agent or a composition that includes an anti-scarring agent. In one aspect, the anti-scarring agent is not paclitaxel or a derivative thereof.

Numerous polymeric and non-polymeric delivery systems for use in intraocular implants have been described above.

Methods for coating fibrosis-inhibiting compositions onto or into the implants include: (a) directly affixing to the implants a fibrosis-inhibiting composition (e.g., by either a spraying process or dipping process as described above, with or without a carrier), (b) directly incorporating into the implant a fibrosis-inhibiting composition (e.g., by either a spraying process or dipping process as described above, with or without a carrier (c) by coating the implant with a substance such as a hydrogel which will in turn absorb the fibrosis-inhibiting composition, (d) constructing the implant itself or a portion of the lens with a fibrosis-inhibiting composition, or (e) by covalently binding the fibrosis-inhibiting agent directly to the lens surface or to a linker (small molecule or polymer) that is coated or attached to the implant surface. For these devices, the coating process can be performed in such a manner as to (a) coat the posterior surface of the specific implant, (b) coat the anterior surface of the implant or (c) coat all or parts of both the posterior and anterior surfaces of the device. The protruding arms of the implant can also be coated with the fibrosis-inhibiting agent.

In addition to coating the device with the fibrosis-inhibiting composition, the fibrosis-inhibiting agent can be mixed with the materials that are used to make the device such that the fibrosis-inhibiting agent is incorporated into the final device.

The process of coating these implants with a fibrosis-inhibiting agent or incorporating the fibrosis-inhibiting agent into the implant and the materials selected for these processes are such that they do not significantly alter the refractive index of the intraocular implant or the visible light transmission of the implant or lens.

According to the present invention, any scarring agent described above can be utilized in the practice of this embodiment. Within one embodiment of the invention, intraocular implants may be adapted to release an agent that inhibits one or more of the four general components of the process of fibrosis (or scarring), including: formation of new blood vessels (angiogenesis), migration and proliferation of connective tissue cells (such as fibroblasts or smooth muscle cells), deposition of extracellular matrix (ECM), and remodeling (maturation and organization of the fibrous tissue). By inhibiting one or more of the components of fibrosis (or scarring), the overgrowth of granulation tissue may be inhibited or reduced.

As intraocular implants are made in a variety of configurations and sizes, the exact dose administered will vary with device size, surface area and design. However, certain principles can be applied in the application of this art. Drug dose can be calculated as a function of dose per unit area (of the portion of the device being coated), total dose administered, and appropriate surface concentrations of active drug can be determined. Drugs are to be used at concentrations that range from several times more than to 10%, 5%, or even less than 1% of the concentration typically used in a single chemotherapeutic systemic dose application. Preferably, the drug is released in effective concentrations for a period ranging from 1-90 days.

Several examples of fibrosis-inhibiting agents for use in intraocular implants include the following: cell cycle inhibitor s including (A) anthracyclines (e.g., doxorubicin and mitoxantrone), (B) taxanes (e.g., paclitaxel, TAXOTERE and docetaxel), and (C) podophyllotoxins (e.g., etoposide); (D) immunomodulators (e.g., sirolimus, everolimus, tacrolimus); (E) heat shock protein 90 antagonists (e.g., geldanamycin); (F) HMGCoA reductase inhibitors (e.g., simvastatin); (G) inosine monophosphate dehydrogenase inhibitors (e.g., mycophenolic acid, 1-alpha-25 dihydroxy vitamin D₃); (H)NF kappa B inhibitors (e.g., Bay 11-7082); (I) antimycotic agents (e.g., sulconizole), (J) p38 MAP kinase inhibitors (e.g., SB202190), and (K) and anti-angiogenesis agents (e.g., halofuginone bromide), as well as analogues and derivatives of the aforementioned.

Regardless of the method of application of the drug to the intraocular implant, the exemplary anti-fibrosing agents, used alone or in combination, should be administered under the following dosing guidelines. The total amount (dose) of anti-scarring agent in or on the device may be in the range of about 0.01 μg-10 μg, or 10 μg-10 mg, or 10 mg-250 mg, or 250 mg-1000 mg, or 1000 mg-2500 mg. The dose (amount) of anti-scarring agent per unit area of device surface to which the agent is applied may be in the range of about 0.01 μg/mm²-1 μg/mm², or 1 μg/mm²-10 μg/mm², or 10 μg/mm²-250 μg/mm², 250 μg/mm²-1000 μg/mm², or 1000 μg/mm²-2500 μg/mm².

Provided below are exemplary dosage ranges for various anti-scarring agents that can be used in conjunction with intraocular implants in accordance with the invention. A) Cell cycle inhibitors including doxorubicin and mitoxantrone. Doxorubicin analogues and derivatives thereof: total dose not to exceed 25 mg (range of 0.1 μg to 25 mg); preferred 1 μg to 5 mg. The dose per unit area of 0.01 μg-100 μg per mm²; preferred dose of 0.1 μg/mm²-10 μg/mm². Minimum concentration of 10⁻⁸-10⁻⁴ M of doxorubicin is to be maintained on the device surface. Mitoxantrone and analogues and derivatives thereof: total dose not to exceed 5 mg (range of 0.01 μg to 5 mg); preferred 0.1 μg to 1 mg. The dose per unit area of the device of 0.01 μg-20 μg per mm²; preferred dose of 0.05 μg/mm²-3 μg/mm². Minimum concentration of 10⁻⁸-10⁻⁴ M of mitoxantrone is to be maintained on the device surface. B) Cell cycle inhibitors including Paclitaxel and analogues and derivatives (e.g., docetaxel) thereof: total dose not to exceed 10 mg (range of 0.1 μg to 10 mg); preferred 1 μg to 3 mg. The dose per unit area of the device of 0.1 μg-10 μg per mm²; preferred dose of 0.25 μg/mm²-5 μg/mm². Minimum concentration of 10⁻⁸-10⁻⁴ M of paclitaxel is to be maintained on the device surface. (C) Cell cycle inhibitors such as podophyllotoxins (e.g., etoposide): total dose not to exceed 10 mg (range of 0.1 μg to 10 mg); preferred 1 μg to 3 mg. The dose per unit area of the device of 0.1 μg-10 μg per mm²; preferred dose of 0.25 μg/mm²-5 μg/mm². Minimum concentration of 10⁻⁸-10⁻⁴ M of etoposide is to be maintained on the device surface. (D) Immunomodulators including sirolimus and everolimus. Sirolimus (i.e., rapamycin, RAPAMUNE): Total dose not to exceed 10 mg (range of 0.1 μg to 10 mg); preferred 10 μg to 1 mg. The dose per unit area of 0.1 μg-100 μg per mm²; preferred dose of 0.5 μg/mm²-10 μg/mm². Minimum concentration of 10⁻⁸-10⁻⁴ M is to be maintained on the device surface. Everolimus and derivatives and analogues thereof: Total dose should not exceed 10 mg (range of 0.1 μg to 10 mg); preferred 10 μg to 1 mg. The dose per unit area of 0.1 μg-100 μg per mm² of surface area; preferred dose of 0.3 μg/mm²-10 μg/mm². Minimum concentration of 10⁻⁸-10⁻⁴ M of everolimus is to be maintained on the device surface. (E) Heat shock protein 90 antagonists (e.g., geldanamycin) and analogues and derivatives thereof: total dose not to exceed 20 mg (range of 0.1 μg to 20 mg); preferred 1 μg to 5 mg. The dose per unit area of the device of 0.1 μg-10 μg per mm²; preferred dose of 0.25 μg/mm²-5 μg/mm². Minimum concentration of 10⁻⁸-10⁻⁴ M of geldanamycin is to be maintained on the device surface. (F) HMGCoA reductase inhibitors (e.g., simvastatin) and analogues and derivatives thereof: total dose not to exceed 200 mg (range of 10.0 μg to 200 mg); preferred 10 μg to 100 mg. The dose per unit area of the device of 1.0 μg-1000 μg per mm²; preferred dose of 2.5 μg/mm²-500 μg/mm². Minimum concentration of 10⁻⁸-10⁻³ M of simvastatin is to be maintained on the device surface. (G) Inosine monophosphate dehydrogenase inhibitors (e.g., mycophenolic acid, 1-alpha-25 dihydroxy vitamin D₃) and analogues and derivatives thereof: total dose not to exceed 200 mg (range of 10.0 μg to 200 mg); preferred 10 μg to 100 mg. The dose per unit area of the device of 1.0 μg-1000 μg per mm²; preferred dose of 2.5 μg/mm²-500 μg/mm². Minimum concentration of 10⁻⁸-10⁻³ M of mycophenolic acid is to be maintained on the device surface. (H)NF kappa B inhibitors (e.g., Bay 11-7082) and analogues and derivatives thereof: total dose not to exceed 200 mg (range of 1.0 μg to 200 mg); preferred 1 μg to 50 mg. The dose per unit area of the device of 1.0 μg-100 μg per mm²; preferred dose of 2.5 μg/mm²-50 μg/mm². Minimum concentration of 10⁻⁸-10⁻⁴ M of Bay 11-7082 is to be maintained on the device surface. (I) Antimycotic agents (e.g., sulconizole) and analogues and derivatives thereof: total dose not to exceed 200 mg (range of 10.0 μg to 200 mg); preferred 10 μg to 100 mg. The dose per unit area of the device of 1.0 μg-1000 μg per mm²; preferred dose of 2.5 μg/mm²-500 μg/mm². Minimum concentration of 10⁻⁸-10⁻³ M of sulconizole is to be maintained on the device surface. (J) p38 MAP kinase inhibitors (e.g., SB202190) and analogues and derivatives thereof: total dose not to exceed 200 mg (range of 10.0 μg to 200 mg); preferred 10 μg to 100 mg. The dose per unit area of the device of 1.0 μg-1000 μg per mm²; preferred dose of 2.5 μg/mm²-500 μg/mm². Minimum concentration of 10⁻⁸-10⁻³ M of SB202190 is to be maintained on the device surface. (K) Anti-angiogenic agents (e.g., halofuginone bromide) and analogues and derivatives thereof: total dose not to exceed 10 mg (range of 0.1 μg to 10 mg); preferred 1 μg to 3 mg. The dose per unit area of the device of 0.1 μg-10 μg per mm²; preferred dose of 0.25 μg/mM²-5 μg/mm². Minimum concentration of 10⁻⁸-10⁻⁴ M of halofuginone bromide is to be maintained on the device surface.

Hypertrophic Scars and Keloids

In another aspect, the present invention provides for the combination of an anti-scarring agent and a device for use in treating hypertrophic scars and keloids.

Hypertrophic scars and keloids are the result of an excessive fibroproliferative wound healing response. Briefly, healing of wounds and scar formation occurs in three phases: inflammation, proliferation, and maturation. The first phase, inflammation, occurs in response to an injury which is severe enough to break the skin. During this phase, which lasts 3 to 4 days, blood and tissue fluid form an adhesive coagulum and fibrinous network which serves to bind the wound surfaces together. This is then followed by a proliferative phase in which there is ingrowth of capillaries and connective tissue from the wound edges, and closure of the skin defect. Finally, once capillary and fibroblastic proliferation has ceased, the maturation process begins wherein the scar contracts and becomes less cellular, less vascular, and appears flat and white. This final phase may take between 6 and 12 months. If too much connective tissue is produced and the wound remains persistently cellular, the scar may become red and raised. If the scar remains within the boundaries of the original wound it is referred to as a hypertrophic scar, but if it extends beyond the original scar and into the surrounding tissue, the lesion is referred to as a keloid. Hypertrophic scars and keloids are produced during the second and third phases of scar formation. Several wounds are particularly prone to excessive endothelial and fibroblastic proliferation, including burns, open wounds, and infected wounds. With hypertrophic scars, some degree of maturation occurs and gradual improvement occurs. In the case of keloids however, an actual tumor is produced which can become quite large. Spontaneous improvement in such cases rarely occurs.

A variety of devices for treating hypertrophic scars and keloids have been described. For example, the device may be an external tissue expansion device composed of two suture steel plates with adhesive attached foam cushions which apply constant continuous low grade force to skin and tissue to provide removal of hypertrophic scars and keloids. See, e.g., U.S. Pat. No. 6,254,624. The device may be a masking element which is pressed onto the scar tissue with an adjustable force by means of a pressure control unit and is connected with inflatable or suction members in the masking element. See, e.g., U.S. Pat. No. 6,013,094. The treatment may be a device having locking elements and grasping structures such that the dermal and epidermal layers of a skin wound can be pushed together such that the tissue edges are abutting, such that a wound may be closed with minimal scarring. See, e.g., U.S. Pat. No. 5,591,206.

In another aspect, the hypertrophic scar or keloid may be treated by using a device in conjunction with a coating or sheet that may be used to deliver either anti-scarring agents alone, or anti-scarring compositions as described above. For example, the coating or sheet may be a copolymer composed of a hydrophilic polymer, such as polyethylene glycol, that is bound to a polymer that adsorbs readily to the surfaces of body tissues, such as phenylboronic acid. See, e.g., U.S. Pat. No. 6,596,267. The coating or sheet may be a self-adhering silicone sheet which is impregnated with an antioxidant and/or antimicrobial. See, e.g., U.S. Pat. No. 6,572,878. The coating or sheet may be a wound dressing garment composed of an outer pliable layer and a self-adhesive inner gel lining which serves as a dressing for contacting wounds. See, e.g., U.S. Pat. No. 6,548,728. The coating or sheet may be a liquid composition composed of a film-forming carrier such as a collodion which contains one or more active ingredients such as a topical steroid, silicone gel and vitamin E. See, e.g., U.S. Pat. No. 6,337,076. The coating or sheet may be a bandage with a scar treatment pad with a layer of silicone elastomer or silicone gel. See, e.g., U.S. Pat. Nos. 6,284,941 and 5,891,076.

In another aspect, a medical device may be used in conjunction with an injectable composition that may be directly injected into a hypertrophic scar or keloid, in order to prevent the progression of these lesions. The frequency of injections will depend upon the release kinetics of the polymer used (if present), and the clinical response. This therapy is of particular value in the prophylactic treatment of conditions which are known to result in the development of hypertrophic scars and keloids (e.g., burns), and is preferably initiated after the proliferative phase has had time to progress (approximately 14 days after the initial injury), but before hypertrophic scar or keloid development. For example, an injectable treatment for hypertrophic scars and keloids may include the administration of an effective amount of angiogenesis inhibitor (e.g., fumagillol, thalidomide) as a systemic or local treatment to decrease excessive scarring. See, e.g., U.S. Pat. No. 6,638,949. The injectable treatment may be a cryoprobe containing cryogen whereby it is positioned within the hypertrophic scar or keloid to freeze the tissue. See, e.g., U.S. Pat. No. 6,503,246. The injectable treatment may be a method of locally administering an amount of botulinum toxin in or in close proximity to the skin wound, such that the healing is enhanced. See, e.g., U.S. Pat. No. 6,447,787. The injectable treatment may be a method of administering an antifibrotic amount of fluoroquinolone to prevent or treat scar tissue formation. See, e.g., U.S. Pat. No. 6,060,474. The injectable treatment may be a composition of an effective amount of calcium antagonist and protein synthesis inhibitor sufficient to cause matrix degradation at a scar site so as to control scar formation. See, e.g., U.S. Pat. No. 5,902,609. The injectable treatment may be a composition of non-biodegradable microspheres with a substantial surface charge in a pharmaceutically acceptable carrier. See, e.g., U.S. Pat. No. 5,861,149. The injectable treatment may be a composition of endothelial cell growth factor and heparin which may be administered topically or by intralesional injection. See, e.g., U.S. Pat. No. 5,500,409.

Treatments and devices used for hypertrophic scars and keloids, which may be combined with one or more agents according to the present invention, include commercially available products. Representative products include, for example, PROXIDERM External Tissue Expansion product for wound healing from Progressive Surgical Products (Westbury, N.Y.), CICA-CARE Gel Sheet dressing product from Smith & Nephew Healthcare Ltd. (India), and MEPIFORM Self-Adherent Silicone Dressing from Molnlycke Health Care (Eddystone, Pa.).

In one aspect, devices for the treatment of hypertrophic scars and keloids may be combined with a topical or injectable composition that includes an anti-scarring agent and a polymeric carrier suitable for application on or into hypertrophic scars or keloids. Incorporation of a fibrosis-inhibiting agent into a topical formulation or an injectable formulation is one approach to treat this condition. The topical formulation can be in the form of a solution, a suspension, an emulsion, a gel, an ointment, a cream, film or mesh. The injectable formulation can be in the form of a solution, a suspension, an emulsion or a gel. Polymeric and non-polymeric components that can be used to prepare these topical or injectable compositions are described above.

In another embodiment, the therapeutic agent can be incorporated into a secondary carrier (e.g., micelles, liposomes, emulsions, microspheres, nanospheres etc, as described above). Microsphere and nanospheres may include degradable polymers. Degradable polymers that can be used include poly(hydroxyl esters) (e.g., PLGA, PLA, PCL, and the like) as well as polyanhydrides, polyorthoesters and polysaccharides (e.g., chitosan and alginates).

According to the present invention, any fibrosis-inhibiting agent described above can be utilized in the practice of this embodiment. Within one embodiment of the invention, devices for the treatment of hypertrophic scars and keloids may be adapted to release an agent that inhibits one or more of the four general components of the process of fibrosis (or scarring), including: formation of new blood vessels (angiogenesis), migration and proliferation of connective tissue cells (such as fibroblasts or smooth muscle cells), deposition of extracellular matrix (ECM), and remodeling (maturation and organization of the fibrous tissue). By inhibiting one or more of the components of fibrosis (or scarring), the overgrowth of granulation tissue may be inhibited or reduced.

As devices for preventing hypertrophic scarring or keloids are made in a variety of configurations and sizes, the exact dose administered will vary with device size, surface area and design. However, certain principles can be applied in the application of this art. Drug dose can be calculated as a function of dose per unit area (of the portion of the device being coated), total dose administered, and appropriate surface concentrations of active drug can be determined. Drugs are to be used at concentrations that range from several times more than to 10%, 5%, or even less than 1% of the concentration typically used in a single chemotherapeutic systemic dose application. Preferably, the drug is released in effective concentrations for a period ranging from 1-90 days.

Several examples of fibrosis-inhibiting agents for use devices for treating hypertrophic scars and keloids include the following: cell cycle inhibitors including (A) anthracyclines (e.g., doxorubicin and mitoxantrone), (B) taxanes (e.g., paclitaxel, TAXOTERE and docetaxel), and (C) podophyllotoxins (e.g., etoposide); (D) immunomodulators (e.g., sirolimus, everolimus, tacrolimus); (E) heat shock protein 90 antagonists (e.g., geldanamycin); (F) HMGCoA reductase inhibitors (e.g., simvastatin); (G) inosine monophosphate dehydrogenase inhibitors (e.g., mycophenolic acid, 1-alpha-25 dihydroxy vitamin D₃); (H)NF kappa B inhibitors (e.g., Bay 11-7082); (I) antimycotic agents (e.g., sulconizole), (J) p38 MAP kinase inhibitors (e.g., SB202190), and (K) and anti-angiogenesis agents (e.g., halofuginone bromide), as well as analogues and derivatives of the aforementioned.

Regardless of the method of application of the drug to the device, the exemplary anti-fibrosing agents, used alone or in combination, should be administered under the following dosing guidelines. The total amount (dose) of anti-scarring agent in or on the device may be in the range of about 0.01 μg-10 μg, or 10 μg-10 mg, or 10 mg-250 mg, or 250 mg-1000 mg, or 1000 mg-2500 mg. The dose (amount) of anti-scarring agent per unit area of device surface to which the agent is applied may be in the range of about 0.01 μg/mm²-1 μg/mm², or 1 μg/mm²-10 μg/mm², or 10 μg/mm²-250 μg/mm², 250 μg/mm²-1000 μg/mm², or 1000 μg/mm²-2500 μg/mm².

Provided below are exemplary dosage ranges for various anti-scarring agents that can be used in conjunction with devices for treating hypertrophic scars and keloids in accordance with the invention. A) Cell cycle inhibitors including doxorubicin and mitoxantrone. Doxorubicin analogues and derivatives thereof: total dose not to exceed 250 mg (range of 1.0 μg to 250 mg); preferred 1 μg to 100 mg. The dose per unit area of 0.01 μg-500 μg per mm²; preferred dose of 0.1 μg/mm²-100 μg/mm². Minimum concentration of 10⁻⁸-10⁻⁴ M of doxorubicin is to be maintained on the device surface. Mitoxantrone and analogues and derivatives thereof: total dose not to exceed 200 mg (range of 1.0 μg to 200 mg); preferred 0.1 μg to 75 mg. The dose per unit area of the device of 0.01 μg-300 μg per mm²; preferred dose of 0.05 μg/mm²-75 μg/mm². Minimum concentration of 10⁻⁸-10⁻⁴ M of mitoxantrone is to be maintained on the device surface. B) Cell cycle inhibitors including Paclitaxel and analogues and derivatives (e.g., docetaxel) thereof: total dose not to exceed 250 mg (range of 1.0 μg to 250 mg); preferred 1 μg to 100 mg. The dose per unit area of the device of 0.1 μg-500 μg per mm²; preferred dose of 0.25 μg/mm²-100 μg/mm². Minimum concentration of of paclitaxel is to be maintained on the device surface. (C) Cell cycle inhibitors such as podophyllotoxins (e.g., etoposide): total dose not to exceed 250 mg (range of 1.0 μg to 250 mg); preferred 1 μg to 100 mg. The dose per unit area of the device of 0.1 μg-500 μg per mm²; preferred dose of 0.25 μg/mm²-100 μg/mm². Minimum concentration of 10⁻⁸-10⁻⁴ M of etoposide is to be maintained on the device surface. (D) Immunomodulators including sirolimus and everolimus. Sirolimus (i.e., rapamycin, RAPAMUNE): Total dose not to exceed 250 mg (range of 1.0 μg to 250 mg); preferred 1 μg to 100 mg. The dose per unit area of the device of 0.1 μg-500 μg per mm²; preferred dose of 0.25 μg/mm²-100 μg/mm². Minimum concentration of 10⁻⁸-10⁻⁴ M is to be maintained on the device surface. Everolimus and derivatives and analogues thereof: Total dose should not exceed 250 mg (range of 1.0 μg to 250 mg); preferred 1 μg to 100 mg. The dose per unit area of the device of 0.1 μg-500 μg per mm²; preferred dose of 0.25 μg/mm²-100 μg/mm². Minimum concentration of 10⁻⁸-10⁻⁴ M of everolimus is to be maintained on the device surface. (E) Heat shock protein 90 antagonists (e.g., geldanamycin) and analogues and derivatives thereof: total dose not to exceed 250 mg (range of 1.0 μg to 250 mg); preferred 1 μg to 100 mg. The dose per unit area of the device of 0.1 μg-500 μg per mm²; preferred dose of 0.25 μg/mm²-100 μg/mm². Minimum concentration of 10⁻⁸-10⁻⁴ M of geldanamycin is to be maintained on the device surface. (F) HMGCoA reductase inhibitors (e.g., simvastatin) and analogues and derivatives thereof: total dose not to exceed 2000 mg (range of 10.0 μg to 2000 mg); preferred 10 μg to 300 mg. The dose per unit area of the device of 1.0 μg-1000 μg per mm²; preferred dose of 2.5 μg/mm²-500 μg/mm². Minimum concentration of 10⁻⁸-10⁻³ M of simvastatin is to be maintained on the device surface. (G) Inosine monophosphate dehydrogenase inhibitors (e.g., mycophenolic acid, 1-alpha-25 dihydroxy vitamin D₃) and analogues and derivatives thereof: total dose not to exceed 2000 mg (range of 10.0 μg to 2000 mg); preferred 10 μg to 300 mg. The dose per unit area of the device of 1.0 μg-1000 μg per mm²; preferred dose of 2.5 μg/mm²-500 μg/mm². Minimum concentration of 10⁻⁸-10⁻³ M of mycophenolic acid is to be maintained on the device surface. (H)NF kappa B inhibitors (e.g., Bay 11-7082) and analogues and derivatives thereof: total dose not to exceed 200 mg (range of 1.0 μg to 200 mg); preferred 1 μg to 50 mg. The dose per unit area of the device of 1.0 μg-100 μg per mm²; preferred dose of 2.5 μg/mm²-50 μg/mm². Minimum concentration of 10⁻⁸-10⁻⁴ M of Bay 11-7082 is to be maintained on the device surface. (I) Antimycotic agents (e.g., sulconizole) and analogues and derivatives thereof: total dose not to exceed 2000 mg (range of 10.0 μg to 2000 mg); preferred 10 μg to 300 mg. The dose per unit area of the device of 1.0 μg-1000 μg per mm²; preferred dose of 2.5 μg/mm²-500 μg/mm². Minimum concentration of 10⁻⁸-10⁻³ M of sulconizole is to be maintained on the device surface. (J) p38 MAP kinase inhibitors (e.g., SB202190) and analogues and derivatives thereof: total dose not to exceed 2000 mg (range of 10.0 μg to 2000 mg); preferred 10 μg to 300 mg. The dose per unit area of the device of 1.0 μg-1000 μg per mm²; preferred dose of 2.5 μg/mm²-500 μg/mm². Minimum concentration of 10⁻⁸-10⁻³ M of SB202190 is to be maintained on the device surface. (K) Anti-angiogenic agents (e.g., halofuginone bromide) and analogues and derivatives thereof: total dose not to exceed 10 mg (range of 0.1 μg to 10 mg); preferred 1 μg to 3 mg. The dose per unit area of the device of 0.1 μg-10 μg per mm²; preferred dose of 0.25 μg/mm²-5 μg/mm². Minimum concentration of 10⁻⁸-10⁻⁴ M of halofuginone bromide is to be maintained on the device surface.

Vascular Grafts

In one aspect, the present invention provides for the combination of an anti-scarring agent and a vascular graft. Vascular graft devices that include a fibrosis-inhibiting agent are capable of inhibiting or reducing the overgrowth of granulation tissue, which can improve the clinical efficacy of these devices.

The vascular graft may be an extravascular graft or an intravascular (i.e., endoluminal) graft. The vascular graft may be, without limitation, in the form of a peripheral bypass application or a coronary bypass application. Vascular grafts may be used to replace or substitute damaged or diseased veins and arteries, including, without limitation, blood vessels damaged by aneurysms, intimal hyperplasia and thrombosis. Vascular grafts may also be used to provide access to blood vessels, for example, for hemodialysis access. Vascular grafts are implanted, for example, to provide an alternative conduit for blood flow through damaged or diseased areas in veins and arteries, including, without limitation, blood vessels damaged by aneurysms, intimal hyperplasia and thrombosis, however, the graft may lead to further complications, including, without limitation, infections, inflammation, thrombosis and intimal hyperplasia. The lack of long-term patency with vascular grafts may be due, for example, to surgical injury and abnormal hemodynamics and material mismatch at the suture line. Typically, further disease (e.g., restenosis) of the vessel occurs along the bed of the artery.

Some forms of improvements to vascular grafts have been made in an attempt to reduce the restenosis that occurs at the anastomosis site. Improvements include: (a) using a Miller cuff, which is a small piece of natural vein to make a short cuff that is joined by stitching it to the artery opening and the prosthetic graft; (b) using a flanged graft whereby the graft has a terminal skirt or cuff that facilitates an end-to-side anastomosis; (c) using a graft with an enlarged chamber having a large diameter for suture at the anastomosis site; and (d) using a graft that dispensing an agent that prevents thrombosis and/or intimal hyperplasia.

Representative examples of vascular grafts include, without limitation, synthetic bypass grafts (e.g., femoral-popliteal, femoral-femoral, axillary-femoral, and the like), vein grafts (e.g., peripheral and coronary), and internal mammary (e.g., coronary) grafts, bifurcated vascular grafts, intraluminal grafts, endovascular grafts and prosthetic grafts. Synthetic grafts can be made from a variety of polymeric materials, such as, for example, polytetrafluoroethylene (e.g., ePTFE), polyesters such as DACRON, polyurethanes, and combinations of polymeric materials.

Endoluminal vascular grafts may be used to treat aneurysms. For example, the vascular graft may be composed of a tubular graft with two tubular self-expanding stents that may be implanted for the treatment of aneurysms by means of minimally invasive procedures. See, e.g., U.S. Pat. No. 6,168,620. The vascular graft may be composed of a flexible tubular body and a compressible frame positioned against the tubular body for support which has pores on the surface to promote ingrowth. See, e.g., U.S. Pat. No. 5,693,088. The vascular graft may be bifurcated endovascular graft having a tubular trunk and two tubular limbs. See, e.g., U.S. Pat. No. 6,454,796. The vascular graft may be a kink-resistant endoluminal bifurcated graft having two separate lumens contacted by a single lumen section. See, e.g., U.S. Pat. No. 6,551,350. The vascular graft may be an intraluminal tube composed of ePTFE that has a seamline formed by overlapping the edges such that the microstructure fibrils are oriented in perpendicular directions. See, e.g., U.S. Pat. No. 5,718,973.

In another aspect, the vascular graft may be used as a conduit to bypass vascular stenosis or other vascular abnormalities. For example, the vascular graft may be composed of a porous material having a layer of porous hollow fibers positioned along the inner surface which allows for tissue growth while inhibiting bleeding during the healing process. See, e.g., U.S. Pat. No. 5,024,671. The vascular graft may be a flexible, monolithic, reinforced polymer tube having a microporous ePTFE tubular member and external ePTFE rib members projecting outwardly from the outer wall. See, e.g., U.S. Pat. No. 5,609,624. The vascular graft may be composed of a tubular wall having longitudinally extending pleats that respond flexurally to changes in blood pressure while maintaining high compliance with reduced kinking. See, e.g., U.S. Pat. No. 5,653,745. The vascular graft may be a radially supported ePTFE tube that is reinforced with greater density ring-shaped regions. See, e.g., U.S. Pat. No. 5,747,128. The vascular graft may be porous PTFE tubing composed of a microstructure of nodes interconnected by fibrils which has a coating of elastomer on the outer wall. See, e.g., U.S. Pat. Nos. 5,152,782 and 4,955,899. The vascular graft may be a plurality of polymeric fibers knitted together composed of at least three different fibers in which two fibers are absorbable and one is non-absorbable. See, e.g., U.S. Pat. Nos. 4,997,440; 4,871,365 and 4,652,264.

In another aspect, the vascular graft may be modified to reduce thrombus formation or intimal hyperplasia at the anastomotic site. For example, the vascular graft may have an enlarged chamber having a first diameter parallel to the axis of the tubular wall and a second diameter transverse to the axis of the tube. See, e.g., U.S. Pat. No. 6,589,278. The vascular graft may have a flanged skirt or cuff section with facilitates an end-to-side anastomosis directly between the artery and the end of the flanged bypass graft. See, e.g., U.S. Pat. No. 6,273,912. The vascular graft may be composed of a tubular wall having a non-thrombogenic agent within the luminal layer and a thrombogenic layer forming the exterior of the vascular graft. See, e.g., U.S. Pat. No. 6,440,166. The vascular graft may be composed of a smooth luminal surface made of ePTFE with a small pore size to reduce adherence of occlusive blood components. See, e.g., U.S. Pat. No. 6,517,571. The vascular graft may be composed of hollow tubing that contains drug that is helically wrapped around the outer wall of a porous ePTFE graft whereby drug is dispensed by infusion through the porous interstices of the graft wall. See, e.g., U.S. Pat. No. 6,355,063.

In another aspect, the vascular graft may be a harvested blood vessel that is used for bypass grafting. For example, vascular grafts may be composed of harvested arterial vessels from a host, such as the internal mammary arteries or inferior epigastric arteries. See, e.g., U.S. Pat. No. 5,797,946. Vascular grafts may also be composed of saphenous veins which may be harvested from the host and used for coronary bypass or peripheral bypass procedures. See, e.g., U.S. Pat. No. 6,558,313.

Other examples of vascular grafts are described in U.S. Pat. Nos. 3,096,560, 3,805,301, 3,945,052, 4,140,126, 4,323,525, 4,355,426, 4,475,972, 4,530,113, 4,550,447, 4,562,596, 4,601,718, 4,647,416, 4,878,908, 5,024,671, 5,104,399, 5,116,360, 5,151,105, 5,197,977, 5,282,824, 5,405,379, 5,609,624, 5,693,088, and 5,910,168.

Vascular grafts, which may be combined with one or more agents according to the present invention, include commercially available products. GORE-TEX Vascular Grafts and GORE-TEX INTERING Vascular Grafts are sold by Gore Medical Division (W. L. Gore & Associates, Inc. Newark, Del.). C.R. Bard, Inc. (Murray Hill, N.J.) sells the DISTAFLO Bypass Grafts and IMPRA CARBOFLO Vascular Grafts.

In one aspect, the anti-scarring agent or a composition containing the anti-scarring agent is combined with a vascular graft.

Numerous polymeric and non-polymeric delivery systems for use in vascular grafts have been described above. Methods for incorporating fibrosis-inhibiting agents or fibrosis-inhibiting compositions onto or into the graft include: (a) affixing (directly or indirectly) to the graft a fibrosis-inhibiting composition (e.g., by either a spraying process or dipping process as described above, with or without a carrier), (b) incorporating or impregnating into the graft a fibrosis-inhibiting composition (e.g., by either a spraying process or dipping process as described above, with or without a carrier), (c) by coating the graft with a substance such as a hydrogel which will in turn absorb the fibrosis-inhibiting composition, (d) constructing the graft itself or a portion of the graft with a fibrosis-inhibiting composition, or (e) by covalently binding the fibrosis-inhibiting agent directly to the graft surface or to a linker (small molecule or polymer) that is coated or attached to the graft surface. For these grafts, the coating process can be performed in such a manner as to (a) coat the external surface of the graft, (b) coat the interior (luminal) surface of the graft, or (c) coat all or parts of both the external and internal surfaces of the graft, or (d) coat at least one end of the graft.

The fibrosis-inhibiting agent can be incorporated directly into the coating composition or into a secondary carrier (e.g., micelles, liposomes, emulsions, microspheres, nanospheres etc, as described above). Microsphere and nanospheres may include degradable polymers. Degradable polymers that can be used include poly(hydroxyl esters) (e.g., PLGA, PLA, PCL, and the like) as well as polyanhydrides, polyorthoesters and polysaccharides (e.g., chitosan and alginates).

In yet another embodiment, a gel, paste, thermogel or in situ forming gel that includes a fibrosis-inhibiting agent can be applied in a perivascular manner to the anastomosis produced during implantation of the graft device. Numerous polymeric and non-polymeric delivery systems for use in paste and gel formulations have been described above. The fibrosis-inhibiting agent can be incorporated directly into the gel or paste composition, or the therapeutic agent can be incorporated into a secondary carrier (e.g., micelles, liposomes, emulsions, microspheres, nanospheres etc, as described above).

In another aspect, the fibrosis-inhibiting agent can be incorporated into or onto an implant (e.g., a film or mesh material), which can be used in conjunction with a vascular graft to inhibit scarring at an anastomotic site. For example, a film or mesh material may be placed or wrapped in a perivascular (periadventitial) manner around the outside of the anastomosis at the time of surgery. Film and mesh implants may be used with a various types of vascular grafts, including synthetic bypass grafts (femoral-popliteal, femoral-femoral, axillary-femoral etc.), vein grafts (peripheral and coronary), internal mammary (coronary) grafts or hemodialysis grafts (AV fistulas, AV access grafts). Representative examples of films and meshes are described in further detail below.

In addition to the fibrosis-inhibiting agent, the vascular graft devices compositions for use with vascular graft devices can also further contain an anti-inflammatory agent (e.g., dexamethazone or aspirin) and/or an anti-thrombotic agent (e.g., heparin, heparin complexes, hydrophobic heparin derivatives, dipyridamole, or aspirin). The combination of agents may be coated onto the entire or portions of the vascular graft such that the thrombogenicity and/or fibrosis is reduced or inhibited. In certain embodiments, these agents may be coated onto the vascular graft using biodegradable polymers. For example, polymeric material that forms a gel in the pores and/or on the surface of the graft may be used, such as alginates, chitosan and chitosan sulfate, hyaluronic acid, dextran sulfate, PLURONIC polymers, chain extended PLURONIC polymers, polyester-polyether block copolymers of the various configurations (e.g., MePEG-PLA, PLA-PEG-PLA, and the like).

In one aspect, synthetic vascular grafts are provided that comprise, in addition to the anti-fibrosing agent, a composition in the form of a biodegradable gel. The gel composition can have anti-thrombogenic properties or include an agent having anti-thrombogenic properties, which may or may not be released from the gel composition. Gel coated grafts may reduce or prevent early thrombotic events commonly associated with implantation of synthetic grafts.

Polymeric biodegradable gels may comprise, for example, a chain extended PLURONIC polymer. Chain extended polymers may include a PLURONIC polymer (e.g., F127, F87, or the like) that has been reacted with a difunctional molecule such as succinyl chloride to increase the molecular weight of the polymer and thereby increase the viscosity of the PLURONIC polymer. Chain extended polymers can be dissolved in a solvent and then coated onto the synthetic vascular graft.

Gel compositions may be formed from a combination of small and/or polymeric molecules having two or more electrophilic groups and two or more nucleophilic groups. For example, the formulations may include a combination of a multi-armed PEG molecule in which the terminal hydroxyl groups are activated with succinimidyl moieties and a multi-armed PEG molecule having terminal amino and/or sulfhydryl groups. The multi-armed PEG reagents may be dissolved separately in an appropriate solvent (e.g., aqueous buffer, IPA, dichloromethane, or a combination of solvents) and then sprayed sequentially or simultaneously onto the desired surface of the graft, such that the two components react to produce a crosslinked gel. The solvent may then be removed by air or vacuum drying.

In another embodiment, the composition may be formed from a polymer having two or more succinimidyl groups and a small molecule having two or more amino or sulfhydryl groups (e.g., dilysine). Alternatively, the polymer components can include two or more sulfhydryl groups or amino groups, and the small molecule contains two or more succinimidyl groups.

In yet another embodiment, gel coatings may be produced from polyester-polyether block copolymers of various configurations (e.g., X—Y, X—Y—X or Y—X—Y, R—(Y—X)ₙ, R—(X—Y)ₙ where X is a polyalkylene oxide and Y is a polyester (e.g., polyester can comprise the residues of one or more of the monomers selected from lactide, lactic acid, glycolide, glycolic acid, e-caprolactone, gamma-caprolactone, hydroxyvaleric acid, hydroxybutyric acid, beta-butyrolactone, gamma-butyrolactone, gamma-valerolactone, ?-decanolactone, d-decanolactone, trimethylene carbonate, 1,4-dioxane-2-one or 1,5-dioxepan-2one.), R is a multifunctional initiator and copolymers as well as blends and copolymers thereof.) may be used to form the gel coating.

In one embodiment, the synthetic vascular graft is formed of a porous synthetic material such as expanded PTFE (ePTFE). A coating comprising a gel composition, such as described above, may be applied onto the entire graft or a portion of the graft surface (e.g., the interior surface of the graft or the ends of the graft). Further, the pores of the graft may be either partially or fully filled with the coating composition. The extent to which the coating occupies the pores of the device can be altered by changing the solvent used to dissolve the polymer. For example, a surface coating may be achieved by using a hydrophilic solvent such as water which will not wet the hydrophobic surface of an ePTFE graft. Coating from a solvent such as dichloromethane, which wets an ePTFE surface, can be used to coat the polymer composition onto the inner pore structure of the graft.

The gel formulations may have anti-thrombogenic properties due to the hydrophilicity. Hydrophilic coatings may be physically removed from the surface of the graft over time which may reduce the adhesion of platelets to the graft surface. Additionally, an anti-thrombogenic agent (e.g., heparin, fragments of heparin, organic soluble salts of heparin, sulfonated carbohydrates, warfarin, coumadin, coumarin, heparinoid, danaparoid, argatroban chitosan sulfate, or chondroitin sulfate) may be included into the formulation. In one embodiment, the anti-thrombotic agent(s) may be incorporated into microspheres. Other additives which may be added into gel compositions for use with vascular grafts include buffers, osmolality modifiers, viscosity modifiers, and hydrating agents (e.g., PEG, MePEG, and various sugars).

According to the present invention, any scarring agent described above can be utilized in the practice of this embodiment. Within one embodiment of the invention, vascular grafts may be adapted to release an agent that inhibits one or more of the four general components of the process of fibrosis (or scarring), including: formation of new blood vessels (angiogenesis), migration and proliferation of connective tissue cells (such as fibroblasts or smooth muscle cells), deposition of extracellular matrix (ECM), and remodeling (maturation and organization of the fibrous tissue). By inhibiting one or more of the components of fibrosis (or scarring), the overgrowth of granulation tissue may be inhibited or reduced.

As vascular grafts are made in a variety of configurations and sizes, the exact dose administered will vary with device size, surface area and design. However, certain principles can be applied in the application of this art. Drug dose can be calculated as a function of dose per unit area (of the portion of the device being coated), total dose administered, and appropriate surface concentrations of active drug can be determined. Drugs are to be used at concentrations that range from several times more than to 10%, 5%, or even less than 1% of the concentration typically used in a single chemotherapeutic systemic dose application. Preferably, the drug is released in effective concentrations for a period ranging from 1-90 days.

Several examples of fibrosis-inhibiting agents for use with vascular grafts include the following: cell cycle inhibitors including (A) anthracyclines (e.g., doxorubicin and mitoxantrone), (B) taxanes (e.g., paclitaxel, TAXOTERE and docetaxel), and (C) podophyllotoxins (e.g., etoposide); (D) immunomodulators (e.g., sirolimus, everolimus, tacrolimus); (E) heat shock protein 90 antagonists (e.g., geldanamycin); (F) HMGCoA reductase inhibitors (e.g., simvastatin); (G) inosine monophosphate dehydrogenase inhibitors (e.g., mycophenolic acid, 1-alpha-25 dihydroxy vitamin D₃); (H)NF kappa B inhibitors (e.g., Bay 11-7082); (I) antimycotic agents (e.g., sulconizole), (J) p38 MAP kinase inhibitors (e.g., SB202190), and (K) and anti-angiogenesis agents (e.g., halofuginone bromide), as well as analogues and derivatives of the aforementioned.

Regardless of the method of application of the drug to the vascular graft, the exemplary anti-fibrosing agents, used alone or in combination, should be administered under the following dosing guidelines. The total amount (dose) of anti-scarring agent in or on the device may be in the range of about 0.01 μg-10 μg, or 10 μg-10 mg, or 10 mg-250 mg, or 250 mg-1000 mg, or 1000 mg-2500 mg. The dose (amount) of anti-scarring agent per unit area of device surface to which the agent is applied may be in the range of about 0.01 μg/mm²-1 μg/mm², or 1 μg/mm²-10 μg/mm², or 10 μg/mm²-250 μg/mm², 250 μg/mm²-1000 μg/mm², or 1000 μg/mm²-2500 μg/mm².

Provided below are exemplary dosage ranges for various anti-scarring agents that can be used in conjunction with vascular graft devices in accordance with the invention. A) Cell cycle inhibitors including doxorubicin and mitoxantrone. Doxorubicin analogues and derivatives thereof: total amount of drug on the device not to exceed 25 mg (range of 0.1 μg to 25 mg); preferred 1 μg to 5 mg. The dose per unit area of 0.01 μg-100 μg per mm²; preferred dose of 0.1 μg/mm²-10 μg/mm². Minimum concentration of 10⁻⁸-10⁻⁴ M of doxorubicin is to be maintained on the device surface. Mitoxantrone and analogues and derivatives thereof: total amount of drug on the device not to exceed 5 mg (range of 0.01 μg to 5 mg); preferred 0.1 μg to 1 mg. The dose per unit area of the device of 0.01 μg-20 μg per mm²; preferred dose of 0.05 μg/mm²-3 μg/mm². Minimum concentration of 10⁻⁸-10⁻⁴ M of mitoxantrone is to be maintained on the device surface. B) Cell cycle inhibitors including Paclitaxel and analogues and derivatives (e.g., docetaxel) thereof: total amount of drug on the device not to exceed 10 mg (range of 0.1 μg to 10 mg); preferred 1 μg to 3 mg. The dose per unit area of the device of 0.1 μg-10 μg per mm²; preferred dose of 0.25 μg/mm²-5 μg/mm². Minimum concentration of 10⁻⁸-10⁻⁴ M of paclitaxel is to be maintained on the device surface. (C) Cell cycle inhibitors such as podophyllotoxins (e.g., etoposide): total amount of drug on the device not to exceed 10 mg (range of 0.1 μg to 10 mg); preferred 1 μg to 3 mg. The dose per unit area of the device of 0.1 μg-10 μg per mm²; preferred dose of 0.25 μg/mm²-5 μg/mm². Minimum concentration of 10⁻⁸-10⁻⁴ M of etoposide is to be maintained on the device surface. (D) Immunomodulators including sirolimus and everolimus. Sirolimus (i.e., rapamycin, RAPAMUNE): Total amount of drug on the device not to exceed 10 mg (range of 0.1 μg to 10 mg); preferred 10 μg to 1 mg. The dose per unit area of 0.1 μg-100 μg per mm²; preferred dose of 0.5 μg/mm²-10 μg/mm². Minimum concentration of 10⁻⁸-10⁻⁴ M is to be maintained on the device surface. Everolimus and derivatives and analogues thereof: Total dose should not exceed 10 mg (range of 0.1 μg to 10 mg); preferred 10 μg to 1 mg. The dose per unit area of 0.1 μg-100 μg per mm² of surface area; preferred dose of 0.3 μg/mm²-10 μg/mm². Minimum concentration of 10⁻⁸-10⁻⁴ M of everolimus is to be maintained on the device surface. (E) Heat shock protein 90 antagonists (e.g., geldanamycin) and analogues and derivatives thereof: total amount of drug on the device not to exceed 20 mg (range of 0.1 μg to 20 mg); preferred 1 μg to 5 mg. The dose per unit area of the device of 0.1 μg-10 μg per mm²; preferred dose of 0.25 μg/mm²-5 μg/mm². Minimum concentration of 10⁻⁸-10⁻⁴ M of geldanamycin is to be maintained on the device surface. (F) HMGCoA reductase inhibitors (e.g., simvastatin) and analogues and derivatives thereof: total amount of drug on the device not to exceed 2000 mg (range of 10.0 μg to 2000 mg); preferred 10 μg to 300 mg. The dose per unit area of the device of 1.0 μg-1000 μg per mm², preferred dose of 2.5 μg/mm²-500 μg/mm². Minimum concentration of 10⁻⁸-10⁻³ M of simvastatin is to be maintained on the device surface. (G) Inosine monophosphate dehydrogenase inhibitors (e.g., mycophenolic acid, 1-alpha-25 dihydroxy vitamin D₃) and analogues and derivatives thereof: total amount of drug on the device not to exceed 2000 mg (range of 10.0 μg to 2000 mg); preferred 10 μg to 300 mg. The dose per unit area of the device of 1.0 μg-1000 μg per mm²; preferred dose of 2.5 μg/mm²-500 μg/mm². Minimum concentration of 10⁻⁸-10⁻³ M of mycophenolic acid is to be maintained on the device surface. (H)NF kappa B inhibitors (e.g., Bay 11-7082) and analogues and derivatives thereof: total amount of drug on the device not to exceed 200 mg (range of 1.0 μg to 200 mg); preferred 1 μg to 50 mg. The dose per unit area of the device of 1.0 μg-100 μg per mm²; preferred dose of 2.5 μg/mm²-50 μg/mm². Minimum concentration of 10⁻⁸-10⁻⁴ M of Bay 11-7082 is to be maintained on the device surface. (1) Antimycotic agents (e.g., sulconizole) and analogues and derivatives thereof: total amount of drug on the device not to exceed 2000 mg (range of 10.0 μg to 2000 mg); preferred 10 μg to 300 mg. The dose per unit area of the device of 1.0 μg-1000 μg per mm preferred dose of 2.5 μg/mm²-500 μg/mm². Minimum concentration of 10⁻⁸-10⁻³ M of sulconizole is to be maintained on the device surface. (J) p38 MAP kinase inhibitors (e.g., SB202190) and analogues and derivatives thereof: total amount of drug on the device not to exceed 2000 mg (range of 10.0 μg to 2000 mg); preferred 10 μg to 300 mg. The dose per unit area of the device of 1.0 μg-1000 μg per mm²; preferred dose of 2.5 μg/mm²-500 μg/mm². Minimum concentration of 10⁻⁸-10⁻³ M of SB202190 is to be maintained on the device surface. (K) Anti-angiogenic agents (e.g., halofuginone bromide) and analogues and derivatives thereof: total dose not to exceed 10 mg (range of 0.1 μg to 10 mg); preferred 1 μg to 3 mg. The dose per unit area of the device of 0.1 μg-10 μg per mm²; preferred dose of 0.25 μg/mm²-5 μg/mm². Minimum concentration of 10⁻⁸-10⁴ M of halofuginone bromide is to be maintained on the device surface.

In addition to those described above (e.g., sirolimus, everolimus, and tacrolimus), several other examples of immunomodulators and appropriate dosages ranges for use with vascular graft devices include the following: (A) Biolimus and derivatives and analogues thereof: Total dose should not exceed 10 mg (range of 0.1 μg to 10 mg); preferred 10 μg to 1 mg. The dose per unit area of 0.1 μg-100 μg per mm² of surface area; preferred dose of 0.3 μg/mm²-10 μg/mm². Minimum concentration of 10⁻⁸-10⁻⁴ M of everolimus is to be maintained on the device surface. (B) Tresperimus and derivatives and analogues thereof: Total dose should not exceed 10 mg (range of 0.1 μg to 10 mg); preferred 10 μg to 1 mg. The dose per unit area of 0.1 μg-100 μg per mm² of surface area; preferred dose of 0.3 μg/mm²-10 μg/mm². Minimum concentration of 10⁻⁸-10⁻⁴ M of tresperimus is to be maintained on the device surface. (C) Auranofin and derivatives and analogues thereof: Total dose should not exceed 10 mg (range of 0.1 μg to 10 mg); preferred 10 μg to 1 mg. The dose per unit area of 0.1 μg-100 μg per mm² of surface area; preferred dose of 0.3 μg/mm²-10 μg/mm². Minimum concentration of 10⁻⁸-10⁻⁴ M of auranofin is to be maintained on the device surface. (D) 27-0-Demethylrapamycin and derivatives and analogues thereof: Total dose should not exceed 10 mg (range of 0.1 μg to 10 mg); preferred 10 μg to 1 mg. The dose per unit area of 0.1 μg-100 μg per mm² of surface area; preferred dose of 0.3 μg/mm²-10 μg/mm². Minimum concentration of 10⁻⁸-10⁻⁴ M of 27-0-Demethylrapamycin is to be maintained on the device surface. (E) Gusperimus and derivatives and analogues thereof: Total dose should not exceed 10 mg (range of 0.1 μg to 10 mg); preferred 10 μg to 1 mg. The dose per unit area of 0.1 μg-100 μg per mm² of surface area; preferred dose of 0.3 μg/mm²-10 μg/mm². Minimum concentration of 10⁻⁸-10⁻⁴ M of gusperimus is to be maintained on the device surface. (F) Pimecrolimus and derivatives and analogues thereof: Total dose should not exceed 10 mg (range of 0.1 μg to 10 mg); preferred 10 μg to 1 mg. The dose per unit area of 0.1 μg-100 μg per mm² of surface area; preferred dose of 0.3 μg/mm²-10 μg/mm². Minimum concentration of 10⁻⁸-10⁻⁴ M of pimecrolimus is to be maintained on the device surface and (G) ABT-578 and analogues and derivatives thereof: Total dose should not exceed 10 mg (range of 0.1 μg to 10 mg); preferred 10 μg to 1 mg. The dose per unit area of 0.1 μg-100 μg per mm² of surface area; preferred dose of 0.3 μg/mm²-10 μg/mm². Minimum concentration of 10⁻⁸-10⁻⁴ M of ABT-578 is to be maintained on the device surface.

In addition to those described above (e.g., paclitaxel, TAXOTERE, and docetaxel), several other examples of anti-microtubule agents and appropriate dosages ranges for use with vascular graft devices include vinca alkaloids such as vinblastine and vincristine sulfate and analogues and derivatives thereof: total dose not to exceed 10 mg (range of 0.1 μg to 10 mg); preferred 1 μg to 3 mg. Dose per unit area of the device of 0.1 μg-10 μg per mm²; preferred dose of 0.25 g/mm²-5 μg/mm². Minimum concentration of 10⁻⁸-10⁻⁴ M of drug is to be maintained on the device surface.

Hemodialysis Access Devices

In one aspect, the present invention provides for the combination of an anti-scarring agent and a hemodialysis access device. Hemodialysis dialysis access devices that include a fibrosis-inhibiting agent are capable of inhibiting or reducing the overgrowth of granulation tissue, which can improve the clinical efficacy of these devices.

Hemodialysis access devices may be used when blood needs to be removed, cleansed and then returned to the body. Hemodialysis regulates the body's fluid and chemical balances as well as removes waste from the blood stream that cannot be cleansed by a normally functioning kidney due to disease or injury. For hemodialysis to occur, the blood may be obtained through a hemodialysis access or vascular access, in which minor surgery is performed to provide access through an AV fistula or AV access graft. These hemodialysis access devices may develop complications, including infections, inflammation, thrombosis and intimal hyperplasia of the associated blood vessels. The lack of long-term patency with hemodialysis access may be due to surgical injury, abnormal hemodynamics and material mismatch at the suture line. Typically, further disease (e.g., restenosis) of the vessel occurs along the bed of the artery and/or at the site of anastomosis.

In addition to the AV fistulas and AV access grafts described above, implantable subcutaneous hemodialysis access systems such as the commercially available catheters, ports, and shunts, may also be used for hemodialysis patients. These access systems may consist of a small metallic or polymeric device or devices implanted underneath the skin. These devices may be connected to flexible tubes, which are inserted into a vessel to allow for blood access.

Representative examples of hemodialysis access devices include, without limitation, AV access grafts, venous catheters, vascular grafts, implantable ports, and AV shunts. Synthetic hemodialysis access devices can be made from metals or polymers, such as polytetrafluoroethylene (e.g., ePTFE), polyesters such as DACRON, polyurethanes, or combinations of these materials.

In one aspect, the hemodialysis access device may be an AV access graft. For example, the AV access graft may be composed of an implantable self-expanding flexible percutaneous stent-graft of open weave construction with ends being compressible and having an elastic layer arranged along a portion of its length. See, e.g., U.S. Pat. Nos. 5,755,775 and 5,591,226. The AV access graft may be composed of a tubular section with a generally constant diameter which tapers towards the venous end. See, e.g., U.S. Pat. No. 6,585,762. The AV access graft may be composed of a two microporous ePTFE tubes that are circumferentially disposed over each other with a polymeric layer interposed between such that the graft is self-sealing and exhibits superior radial tensile strength and suture hole elongation resistance. See, e.g., U.S. Pat. No. 6,428,571. The AV access graft may be composed of a coaxial double lumen tube with an inner and outer tube having a self-sealing, nonbiodegradable, polymeric adhesive between the tubes. See, e.g., U.S. Pat. No. 4,619,641. The AV access graft may be composed of a synthetic fabric having a high external velour profile which is woven or knitted to form a tubular prosthesis which has elastic fibers that allows self-sealing following a punctured state. See, e.g., U.S. Pat. No. 6,547,820. The AV access graft may be of tubular form having a base tube with the ablumenal surface covered with a deflectable material, such as a porous film, which is arranged adjacently to allow movement. See, e.g., U.S. Pat. No. 5,910,168.

In another aspect, the hemodialysis access device may be a catheter system. For example, the catheter system may be composed of a suction and return line that are adapted for disposition in the vascular system of the body and are connected to a subcutaneous connector port. See, e.g., U.S. Pat. Nos. 6,620,118 and 5,989,206. The catheter system may be an apparatus that is used to arterialize a vein by creating an AV fistula by inserting a catheter into a vein and a catheter into an adjacent artery. See, e.g., U.S. Pat. No. 6,464,665. The catheter system may be composed of a hollow sheath that provides percutaneous introduction of fistula-generating vascular catheters through a perforation in a vessel wall, such that the catheters generate an intervascular fistula on-demand between adjacent vessels. See, e.g., U.S. Pat. Nos. 6,099,542 and 5,830,224.

In another aspect, the hemodialysis access device may be used for an AV fistula. For example, the hemodialysis access device may be an AV fistula assembly composed of a synthetic coiled stent graft with helically-extending turns with gaps used to enhance the function of an AV fistula. See, e.g., U.S. Pat. No. 6,585,760.

In another aspect, the hemodialysis access device may be an implantable access port, shunt or valve. These devices may be implanted subcutaneously with communication to the blood supply and accessed using a percutaneous puncture. For example, the hemodialysis access device may be composed of housing having an entry port and an exit port to a passageway which has an elastomeric sealing valve that provides access into the exit port for a needle. See, e.g., U.S. Pat. No. 5,741,228. The hemodialysis access device may be a shunt composed of a slideable valve and flexible lid that has a fluid communication tube between the arterial and venous ends. See, e.g., U.S. Pat. No. 5,879,320. The hemodialysis access device may be a shunt in the form of a junction that has a connector with two legs that are inserted into the native blood vessel and one leg that is adapted for sealing to another blood vessel without punctures. See, e.g., U.S. Pat. No. 6,019,788. The hemodialysis access device may be a surface access double hemostatic valve that may be mounted on the wall of an AV graft for hemodialysis access. See, e.g., U.S. Pat. Nos. 6,004,301 and 6,090,067.

Hemodialysis access devices, which may be combined with one or more agents according to the present invention, include commercially available products. For example, hemodialysis access devices include products, such as the LIFESITE (Vasca Inc., Tewksbury, Mass.) and the DIALOCK catheters from Biolink Corp. (Middleboro, Mass.), VECTRA Vascular Access Grafts and VENAFLO Vascular Grafts from C.R. Bard, Inc. (Murray Hill, N.J.), and GORE-TEX Vascular Grafts and Stretch Vascular Grafts from Gore Medical Division (W. L. Gore & Associates, Inc. Newark, Del.).

In one aspect, the anti-scarring agent or a composition containing the anti-scarring agent is combined with a hemodialysis access device. Numerous polymeric and non-polymeric delivery systems for use in hemodialysis access devices have been described above. Methods for incorporating fibrosis-inhibiting agents or compositions comprising fibrosis-inhibiting agents onto or into the hemodialysis access device include: (a) directly affixing to the hemodialysis access device a fibrosis-inhibiting composition (e.g., by either a spraying process or dipping process as described above, with or without a carrier), (b) directly incorporating into the hemodialysis access device a fibrosis-inhibiting composition (e.g., by either a spraying process or dipping process as described above, with or without a carrier), (c) by coating the hemodialysis access device with a substance such as a hydrogel which will in turn absorb the fibrosis-inhibiting composition, (d) constructing the hemodialysis access device itself or a portion of the graft with a fibrosis-inhibiting composition, or (e) by covalently binding the fibrosis-inhibiting agent directly to the hemodialysis access device surface or to a linker (small molecule or polymer) that is coated or attached to the hemodialysis access device surface. For devices that are coated, the coating process can be performed in such a manner as to (a) coat only the external surface of the device; (b) coat the internal (luminal) surface of the device; or (c) coat all or parts of both the external and internal surfaces.

In another aspect, the fibrosis-inhibiting agent or a composition containing the fibrosis-inhibiting agent can be incorporated into an implant, such as a film or mesh, which can be used in conjunction with a hemodialysis access device to inhibit scarring at the site of an anastomosis or fistula. These films or meshes may be placed or wrapped in a perivascular (periadventitial) manner around the outside of the fistula or anastomosis at the time of surgery. Representative examples of implants (i.e., meshes and films) for use with hemodialysis access devices are described below.

In yet another aspect, a composition in the form of, for example, a gel, paste, thermogel, or in situ forming gel, which includes a fibrosis-inhibiting agent can be applied in a perivascular manner to the fistula or anastomosis produced during implantation of the hemodialysis access device.

The fibrosis-inhibiting agent can be incorporated directly into the gel or paste composition, or the therapeutic agent can be incorporated into a secondary carrier (e.g., micelles, liposomes, emulsions, microspheres, nanospheres etc, as described above) that is then incorporated into the composition that is to be delivered. Microsphere and nanospheres may include degradable polymers. Degradable polymers that can be used include poly (hydroxyl esters) (e.g., PLGA, PLA, PCL, and the like) as well as polyanhydrides, polyorthoesters and polysaccharides (e.g., chitosan and alginates).

In addition to the fibrosis-inhibiting agent, hemodialysis access devices and compositions for use with hemodialysis access devices can also further contain an anti-inflammatory agent (e.g., dexamethazone or aspirin) and/or an anti-thrombotic agent (e.g., heparin, heparin complexes, hydrophobic heparin derivatives, dipyridamole, or aspirin).

According to the present invention, any scarring agent described above can be utilized in the practice of this embodiment. Within one embodiment of the invention, hemodialysis access devices may be adapted to release an agent that inhibits one or more of the four general components of the process of fibrosis (or scarring), including: formation of new blood vessels (angiogenesis), migration and proliferation of connective tissue cells (such as fibroblasts or smooth muscle cells), deposition of extracellular matrix (ECM), and remodeling (maturation and organization of the fibrous tissue). By inhibiting one or more of the components of fibrosis (or scarring), the overgrowth of granulation tissue may be inhibited or reduced.

Several examples of fibrosis-inhibiting agents for use with hemodialysis access devices include the following: cell cycle inhibitors including (A) anthracyclines (e.g., doxorubicin and mitoxantrone), (B) taxanes (e.g., paclitaxel, TAXOTERE and docetaxel), and (C) podophyllotoxins (e.g., etoposide); (D) immunomodulators (e.g., sirolimus, everolimus, tacrolimus); (E) heat shock protein 90 antagonists (e.g., geldanamycin); (F) HMGCoA reductase inhibitors (e.g., simvastatin); (G) inosine monophosphate dehydrogenase inhibitors (e.g., mycophenolic acid, 1-alpha-25 dihydroxy vitamin D₃); (H)NF kappa B inhibitors (e.g., Bay 11-7082); (I) antimycotic agents (e.g., sulconizole), (J) p38 MAP kinase inhibitors (e.g., SB202190), and (K) and anti-angiogenesis agents (e.g., halofuginone bromide), as well as analogues and derivatives of the aforementioned.

As hemodialysis access devices are made in a variety of configurations and sizes, the exact dose administered will vary with device size, surface area and design. However, certain principles can be applied in the application of this art. Drug dose can be calculated as a function of dose per unit area (of the portion of the device being coated), and total amount of drug on the device can be measured and appropriate surface concentrations of active drug can be determined. Drugs are to be used at concentrations that range from several times more than to 10%, 5%, or even less than 1% of the concentration typically used in a single chemotherapeutic systemic dose application. Preferably, the drug is released in effective concentrations for a period ranging from 1-90 days.

Regardless of the method of application of the drug to the device, the exemplary anti-fibrosing agents, used alone or in combination, should be administered under the following dosing guidelines. The total amount (dose) of anti-scarring agent in or on the device may be in the range of about 0.01 μg-10 μg, or 10 μg-10 mg, or 10 mg-250 mg, or 250 mg-1000 mg, or 1000 mg-2500 mg. The dose (amount) of anti-scarring agent per unit area of device surface to which the agent is applied may be in the range of about 0.01 μg/mm²-1 μg/mm², or 1 μg/mm²-10 μg/mm², or 10 μg/mm²-250 μg/mm², 250 μg/mm²-1000 μg/mm², or 1000 μg/mm²-2500 μg/mm².

Provided below are exemplary dosage ranges for various anti-scarring agents that can be used in conjunction with hemodialysis access devices in accordance with the invention. A) Cell cycle inhibitors including doxorubicin and mitoxantrone. Doxorubicin analogues and derivatives thereof: total amount of drug on the device not to exceed 25 mg (range of 0.1 μg to 25 mg); preferred 1 μg to 5 mg. The dose per unit area of 0.01 μg-100 μg per mm²; preferred dose of 0.1 μg/mm²-10 μg/mm². Minimum concentration of 10⁻⁸-10⁴ M of doxorubicin is to be maintained on the device surface. Mitoxantrone and analogues and derivatives thereof: total amount of drug on the device not to exceed 5 mg (range of 0.01 μg to 5 mg); preferred 0.1 μg to 1 mg. The dose per unit area of the device of 0.01 μg-20 μg per mm²; preferred dose of 0.05 μg/mm²-3 μg/mm². Minimum concentration of 10⁻⁸-10⁻⁴ M of mitoxantrone is to be maintained on the device surface for up to 90 days. B) Cell cycle inhibitors including paclitaxel and analogues and derivatives (e.g., docetaxel) thereof: total amount of drug on the device not to exceed 10 mg (range of 0.1 μg to 10 mg); preferred 1 μg to 3 mg. The dose per unit area of the device of 0.1 μg-10 μg per mm²; preferred dose of 0.25 μg/mm²-5 μg/mm². Minimum concentration of 10⁻⁸-10⁻⁴ M of paclitaxel is to be maintained on the device surface for up to 90 days. (C) Cell cycle inhibitors such as podophyllotoxins (e.g., etoposide): total amount of drug on the device not to exceed 10 mg (range of 0.1 μg to 10 mg); preferred 1 μg to 3 mg. The dose per unit area of the device of 0.1 μg-10 μg per mm²; preferred dose of 0.25 μg/mm²-5 μg/mm². Minimum concentration of 10⁻⁸-10⁻⁴ M of etoposide is to be maintained on the device surface for up to 90 days. (D) Immunomodulators including sirolimus and everolimus. Sirolimus (i.e., rapamycin, RAPAMUNE): Total amount of drug on the device not to exceed 10 mg (range of 0.1 μg to 10 mg); preferred 10 μg to 1 mg. The dose per unit area of 0.1 μg-100 μg per mm²; preferred dose of 0.5 μg/mm²-10 μg/mm². Minimum concentration of 10⁻⁸-10⁻⁴ M is to be maintained on the device surface for up to 90 days. Everolimus and derivatives and analogues thereof: Total dose should not exceed 10 mg (range of 0.1 μg to 10 mg); preferred 10 μg to 1 mg. The dose per unit area of 0.1 μg-100 μg per mm² of surface area; preferred dose of 0.3 μg/mm²-10 μg/mm². Minimum concentration of 10⁻⁸-10⁻⁴ M of everolimus is to be maintained on the device surface for up to 90 days. (E) Heat shock protein 90 antagonists (e.g., geldanamycin) and analogues and derivatives thereof: total dose not to exceed 20 mg (range of 0.1 μg to 20 mg); preferred 1 μg to 5 mg. The dose per unit area of the device of 0.1 μg-10 μg per mm²; preferred dose of 0.25 μg/mm²-5 μg/mm². Minimum concentration of 10⁻⁸-10⁻⁴ M of paclitaxel is to be maintained on the device surface for up to 90 days. (F) HMGCoA reductase inhibitors (e.g., simvastatin) and analogues and derivatives thereof: total amount of drug on the device not to exceed 2000 mg (range of 10.0 μg to 2000 mg); preferred 10 μg to 300 mg. The dose per unit area of the device of 1.0 μg-1000 μg per mm²; preferred dose of 2.5 μg/mm²-500 μg/mm². Minimum concentration of 10⁻⁸-10⁻³ M of simvastatin is to be maintained on the device surface for up to 90 days. (G) Inosine monophosphate dehydrogenase inhibitors (e.g., mycophenolic acid, 1-alpha-25 dihydroxy vitamin D₃) and analogues and derivatives thereof: total amount of drug on the device not to exceed 2000 mg (range of 10.0 μg to 2000 mg); preferred 10 μg to 300 mg. The dose per unit area of the device of 1.0 μg-1000 μg per mm²; preferred dose of 2.5 μg/mm²-500 μg/mm². Minimum concentration of 10⁻⁸-10⁻³ M of mycophenolic acid is to be maintained on the device surface for up to 90 days. (H)NF kappa B inhibitors (e.g., Bay 11-7082) and analogues and derivatives thereof: total amount of drug on the device not to exceed 200 mg (range of 1.0 μg to 200 mg); preferred 1 μg to 50 mg. The dose per unit area of the device of 1.0 μg-100 μg per mm²; preferred dose of 2.5 μg/mm²-50 μg/mm². Minimum concentration of 10⁻⁸-10⁻⁴ M of Bay 11-7082 is to be maintained on the device surface for up to 90 days. (I) Antimycotic agents (e.g., sulconizole) and analogues and derivatives thereof: total amount of drug on the device not to exceed 2000 mg (range of 10.0 μg to 2000 mg); preferred 10 μg to 300 mg. The dose per unit area of the device of 1.0 μg-1000 μg per mm²; preferred dose of 2.5 μg/mm²-500 μg/mm². Minimum concentration of 10⁻⁸-10⁻³ M of sulconizole is to be maintained on the device surface for up to 90 days and (J) p38 MAP kinase inhibitors (e.g., SB202190) and analogues and derivatives thereof: total amount of drug on the device not to exceed 2000 mg (range of 10.0 μg to 2000 mg); preferred 10 μg to 300 mg. The dose per unit area of the device of 1.0 μg-1000 μg per mm²; preferred dose of 2.5 μg/mm²-500 μg/mm². Minimum concentration of 10⁻⁸-10⁻⁴ M of SB202190 is to be maintained on the device surface for up to 90 days. (K) Anti-angiogenic agents (e.g., halofuginone bromide) and analogues and derivatives thereof: total dose not to exceed 10 mg (range of 0.1 μg to 10 mg); preferred 1 μg to 3 mg. The dose per unit area of the device of 0.1 μg-10 μg per mm²; preferred dose of 0.25 μg/mm²-5 μg/mm². Minimum concentration of 10⁻⁸-10⁻⁴ M of halofuginone bromide is to be maintained on the device surface.

In addition to those described above (e.g., sirolimus, everolimus, and tacrolimus), several other examples of immunomodulators and appropriate dosages ranges for use with hemodialysis access devices include the following: (A) Biolimus and derivatives and analogues thereof: Total dose should not exceed 10 mg (range of 0.1 μg to 10 mg); preferred 10 μg to 1 mg. The dose per unit area of 0.1 μg-100 μg per mm² of surface area; preferred dose of 0.3 μg/mm²-10 μg/mm². Minimum concentration of 10⁻⁸-10⁻⁴ M of everolimus is to be maintained on the device surface. (B) Tresperimus and derivatives and analogues thereof: Total dose should not exceed 10 mg (range of 0.1 μg to 10 mg); preferred 10 μg to 1 mg. The dose per unit area of 0.1 μg-100 μg per mm² of surface area; preferred dose of 0.3 μg/mm²-10 μg/mm². Minimum concentration of 10⁻⁸-10⁻⁴ M of tresperimus is to be maintained on the device surface. (C) Auranofin and derivatives and analogues thereof: Total dose should not exceed 10 mg (range of 0.1 μg to 10 mg); preferred 10 μg to 1 mg. The dose per unit area of 0.1 μg-100 μg per mm² of surface area; preferred dose of 0.3 μg/mm²-10 μg/mm². Minimum concentration of 10⁻⁸-10⁻⁴ M of auranofin is to be maintained on the device surface. (D) 27-0-Demethylrapamycin and derivatives and analogues thereof: Total dose should not exceed 10 mg (range of 0.1 μg to 10 mg); preferred 10 μg to 1 mg. The dose per unit area of 0.1 μg-100 μg per mm² of surface area; preferred dose of 0.3 μg/mm²-10 μg/mm². Minimum concentration of 10⁻⁸-10⁻⁴ M of 27-0-Demethylrapamycin is to be maintained on the device surface. (E) Gusperimus and derivatives and analogues thereof: Total dose should not exceed 10 mg (range of 0.1 μg to 10 mg); preferred 10 μg to 1 mg. The dose per unit area of 0.1 μg-100 μg per mm² of surface area; preferred dose of 0.3 μg/mm²-10 μg/mm². Minimum concentration of 10⁻⁸-10⁻⁴M of gusperimus is to be maintained on the device surface. (F) Pimecrolimus and derivatives and analogues thereof: Total dose should not exceed 10 mg (range of 0.1 μg to 10 mg); preferred 10 μg to 1 mg. The dose per unit area of 0.1 μg-100 μg per mm² of surface area; preferred dose of 0.3 μg/mm²-10 μg/mm². Minimum concentration of 10⁻⁸-10⁻⁴ M of pimecrolimus is to be maintained on the device surface and (G) ABT-578 and analogues and derivatives thereof: Total dose should not exceed 10 mg (range of 0.1 μg to 10 mg); preferred 10 μg to 1 mg. The dose per unit area of 0.1 μg-100 μg per mm² of surface area; preferred dose of 0.3 μg/mm²-10 μg/mm². Minimum concentration of 10⁻⁸-10⁻⁴ M of ABT-578 is to be maintained on the device surface.

In addition to those described above (e.g., paclitaxel, TAXOTERE, and docetaxel), several other examples of anti-microtubule agents and appropriate dosages ranges for use with hemodialysis access devices include vinca alkaloids such as vinblastine and vincristine sulfate and analogues and derivatives thereof: total dose not to exceed 10 mg (range of 0.1 μg to 10 mg); preferred 1 μg to 3 mg. Dose per unit area of the device of 0.1 μg-10 μg per mm²; preferred dose of 0.25 μg/mm²-5 μg/mm². Minimum concentration of 10⁻⁸-10⁻⁴ M of drug is to be maintained on the device surface.

Films and Meshes

In one aspect, the present invention provides for the combination of an anti-scarring agent and a film or mesh. Incorporation of a fibrosis-inhibiting agent into or onto a film or mesh can minimize fibrosis (or scarring) in the vicinity of the implant and may reduce or prevent the formation of adhesions between the implant and the surrounding tissue. In certain aspects, the film or mesh may be used as a drug-delivery vehicle (e.g., as a perivascular delivery device for the prevention of neointimal hyperplasia at an anastomotic site).

Films or meshes may take a variety of forms including, but not limited to, surgical barriers, surgical adhesion barriers, membranes (e.g., barrier membranes), surgical sheets, surgical patches (e.g., dural patches), surgical wraps (e.g., vascular, perivascular, adventitial, periadventitital wraps, and adventitial sheets), meshes (e.g., perivascular meshes), bandages, liquid bandages, surgical dressings, gauze, fabrics, tapes, surgical membranes, polymer matrices, shells, envelopes, tissue coverings, and other types of surgical matrices, scaffolds, and coatings.

In one aspect, the device comprises or may be in the form of a film. The film may be formed into one of many geometric shapes. Depending on the application, the film may be formed into the shape of a tube or may be a thin, elastic sheet of polymer. Generally, films are less than 5, 4, 3, 2, or 1 mm thick, more preferably less than 0.75 mm, 0.5 mm, 0.25 mm, or, 0.10 mm thick. Films can also be generated of thicknesses less than 50 μm, 25 μm or 10 μm. Films generally are flexible with a good tensile strength (e.g., greater than 50, preferably greater than 100, and more preferably greater than 150 or 200 N/cm²), good adhesive properties (i.e., adheres to moist or wet surfaces), and have controlled permeability. Polymeric films (which may be porous or non-porous) are particularly useful for application to the surface of a device or implant as well as to the surface of tissue, cavity or an organ.

Films may be made by several processes, including for example, by casting, and by spraying, or may be formed at the treatment site in situ. For example, a sprayable formulation may be applied onto the treatment site which then forms into a solid film.

In another aspect, the device may comprise or be in the form of a polymer, wherein at least some of the polymer is in the form of a mesh. A mesh, as used herein, is a material composed of a plurality of fibers or filaments (i.e., a fibrous material), where the fibers or filaments are arranged in such a manner (e.g., interwoven, knotted, braided, overlapping, looped, knitted, interlaced, intertwined, webbed, felted, and the like) so as to form a porous structure. Typically, a mesh is a pliable material, such that it has sufficient flexibility to be wrapped around the external surface of a body passageway or cavity, or a portion thereof. The mesh may be capable of providing support to the structure (e.g., the vessel or cavity wall) and may be adapted to release an amount of the therapeutic agent.

Mesh materials may take a variety of forms. For example, the mesh may be in a woven, knit, or non-woven form and may include fibers or filaments that are randomly oriented relative to each other or that are arranged in an ordered array or pattern. In one embodiment, for example, a mesh may be in the form of a fabric, such as, for example, a knitted, braided, crocheted, woven, non-woven (e.g., a melt-blown or wet-laid) or webbed fabric. In one embodiment, a mesh may include a natural or synthetic biodegradable polymer that may be formed into a knit mesh, a weave mesh, a sprayed mesh, a web mesh, a braided mesh, a looped mesh, and the like. Preferably, a mesh or wrap has intertwined threads that form a porous structure, which may be, for example, knitted, woven, or webbed.

The structure and properties of the mesh used in a device depend on the application and the desired mechanical (i.e., flexibility, tensile strength, and elasticity), degradation properties, and the desired loading and release characteristics for the selected therapeutic agent(s). The mesh should have mechanical properties, such that the device will remain sufficiently strong until the surrounding tissue has healed. Factors that affect the flexibility and mechanical strength of the mesh include, for example, the porosity, fabric thickness, fiber diameter, polymer composition (e.g., type of monomers and initiators), process conditions, and the additives that are used to prepare the material.

Typically, the mesh possesses sufficient porosity to permit the flow of fluids through the pores of the fiber network and to facilitate tissue ingrowth. Generally, the interstices of the mesh should be sufficiently wide apart to allow light visible by eye, or fluids, to pass through the pores. However, materials having a more compact structure also may be used. The flow of fluid through the interstices of the mesh depends on a variety of factors, including, for example, the stitch count or thread density. The porosity of the mesh may be further tailored by, for example, filling the interstices of the mesh with another material (e.g., particles or polymer) or by processing the mesh (e.g., by heating) in order to reduce the pore size and to create non-fibrous areas. Fluid flow through the mesh of the invention will vary depending on the properties of the fluid, such as viscosity, hydrophilicity/hydrophobicity, ionic concentration, temperature, elasticity, pseudoplasticity, particulate content, and the like. Preferably, the interstices do not prevent the release of impregnated or coated therapeutic agent(s) from the mesh, and the interstices preferably do not prevent the exchange of tissue fluid at the application site.

Mesh materials should be sufficiently flexible so as to be capable of being wrapped around all or a portion of the external surface of a body passageway or cavity. Flexible mesh materials are typically in the form of flexible woven or knitted sheets having a thickness ranging from about 25 microns to about 3000 microns; preferably from about 50 to about 1000 microns. Mesh material suitable for wrapping around arteries and veins typically ranges from about 100 to 400 microns in thickness.

The diameter and length of the fibers or filaments may range in size depending on the form of the material (e.g., knit, woven, or non-woven), and the desired elasticity, porosity, surface area, flexibility, and tensile strength. The fibers may be of any length, ranging from short filaments to long threads (i.e., several microns to hundreds of meters in length). Depending on the application, the fibers may have a monofilament or a multifilament construction.

The mesh may include fibers that are of same dimension or of different dimensions, and the fibers may be formed from the same or different types of biodegradable polymers. Woven materials, for example, may include a regular or irregular array of warp and weft strands and may include one type of polymer in the weft direction and another type (having the same or a different degradation profile from the first polymer) in the warp direction. The degradation profile of the weft polymer may be different than or the same as the degradation profile of the warp polymer. Similarly, knit materials may include one or more types (e.g., monofilament, multi-filament) and sizes of fibers and may include fibers made from the same or from different types of biodegradable polymers.

The structure of the mesh (e.g., fiber density and porosity) may impact the amount of therapeutic agent that may be loaded into or onto the device. For example, a fabric having a loose weave characterized by a low fiber density and high porosity will have a lower thread count, resulting in a reduced total fiber volume and surface area. As a result, the amount of agent that may be loaded into or onto, with a fixed carrier: therapeutic agent ratio, the fibers will be lower than for a fabric having a high fiber density and lower porosity. It is preferable that the mesh also should not invoke biologically detrimental inflammatory or toxic response, should be capable of being fully metabolized in the body, have an acceptable shelf life, and be easily sterilized.

The device may include multiple mesh materials in any combination or arrangement. For example, a portion of the device may be a knitted material and another portion may be a woven material. In another embodiment, the device may more than one layer (e.g., a layer of woven material fused to a layer of knitted material or to another layer of the same type or a different type of woven material). In some embodiments, multi-layer constructions (e.g., device having two or more layers of material) may be used, for example, to enhance the performance properties of the device (e.g., for enhancing the rigidity or for altering the porosity, elasticity, or tensile strength of the device) or for increasing the amount of drug loading.

Multi-layer constructions may be useful, for example, in devices containing more than one type of therapeutic agent. For example, a first layer of mesh material may be loaded with one type of agent and a second layer may be loaded with another type of agent. The two layers may be unconnected or connected (e.g., fused together, such as by heat welding or ultrasonic welding) and may be formed of the same type of fabric or from a different type of fabric having a different polymer composition and/or structure.

In certain aspects, a mesh may include portions that are not in the form of a mesh. For example, the device may include the form of a film, sheet, paste, and the like, and combinations thereof. For example, the device may have a multi-layer construction having a film layer that includes the therapeutic agent and one or more layers of mesh material. For example, the film layer may be interposed between two layers of mesh or may be disposed on just one side the mesh material. The film layer may include a first therapeutic agent, whereas one or more of the layers of mesh may include the same or a different agent. In another embodiment, the device includes at least two layers of mesh. In one aspect, at least two of the at least two layers of mesh are fused together.

In one aspect, multilayer devices are provided that may further include a film layer. The film layer may reside between two of the at least two layers of mesh. In yet another embodiment, a delivery device is described that includes a mesh, wherein the mesh includes a biodegradable polymer and a first therapeutic agent. The device may further include a film that includes a second therapeutic agent, which may have the same or a different composition than the first therapeutic agent. For example, in one embodiment, a device suitable for wrapping around a vein or artery includes a layer of mesh and a film layer loaded with a therapeutic agent. The device may be wrapped around a body passageway or cavity, such that the film layer contacts the external surface of the passageway or cavity. Thus, the device may deliver the appropriate dosage of agent and may provide sufficient mechanical strength to improve and maintain the structural integrity of the body passageway or cavity.

In one aspect, the mesh or film includes a polymer. The polymer may be a biodegradable polymer. Biodegradable compositions that may be used to prepare the mesh include polymers that comprise albumin, collagen, hyaluronic acid and derivatives, sodium alginate and derivatives, chitosan and derivatives gelatin, starch, cellulose polymers (for example methylcellulose, hydroxypropylcellulose, hydroxypropylmethylcellulose, carboxymethylcellulose, cellulose acetate phthalate, cellulose acetate succinate, hydroxypropylmethylcellulose phthalate), casein, dextran and derivatives, polysaccharides, poly(caprolactone), fibrinogen, poly(hydroxyl acids), poly(L-lactide) poly(D,L lactide), poly(D,L-lactide-co-glycolide), poly(L-lactide-co-glycolide), copolymers of lactic acid and glycolic acid, copolymers of ε-caprolactone and lactide, copolymers of glycolide and ε-caprolactone, copolymers of lactide and 1,4-dioxane-2-one, polymers and copolymers that include one or more of the residue units of the monomers D-lactide, L-lactide, D,L-lactide, glycolide, ε-caprolactone, trimethylene carbonate, 1,4-dioxane-2-one or 1,5-dioxepan-2-one, poly(glycolide), poly(hydroxybutyrate), poly(alkylcarbonate) and poly(orthoesters), polyesters, poly(hydroxyvaleric acid), polydioxanone, poly(ethylene terephthalate), poly(malic acid), poly(tartronic acid), polyanhydrides, polyphosphazenes, poly(amino acids). These compositions include copolymers of the above polymers as well as blends and combinations of the above polymers. (see generally, Ilium, L., Davids, S. S. (eds.) “Polymers in Controlled Drug Delivery” Wright, Bristol, 1987; Arshady, J. Controlled Release 17: 1-22, 1991; Pitt, Int. J. Phar. 59: 173-196, 1990; Holland et al., J. Controlled Release 4: 155-0180, 1986).

In one aspect, the mesh or film includes a biodegradable or resorbable polymer that is formed from one or more monomers selected from the group consisting of lactide, glycolide, e-caprolactone, trimethylene carbonate, 1,4-dioxan-2-one, 1,5-dioxepan-2-one, 1,4-dioxepan-2-one, hydroxyvalerate, and hydroxybutyrate. In one aspect, the polymer may include, for example, a copolymer of a lactide and a glycolide. In another aspect, the polymer includes a poly(caprolactone). In yet another aspect, the polymer includes a poly(lactic acid), poly(L-lactide)/poly(D,L-Lactide) blends or copolymers of L-lactide and D,L-lactide. In yet another aspect, the polymer includes a copolymer of lactide and e-caprolactone. In yet another aspect, the polymer includes a polyester (e.g., a poly(lactide-co-glycolide). The poly(lactide-co-glycolide) may have a lactide:glycolide ratio ranges from about 20:80 to about 2:98, a lactide:glycolide ratio of about 10:90, or a lactide:glycolide ratio of about 5:95. In one aspect, the poly(lactide-co-glycolide) is poly(L-lactide-co-glycolide). Other examples of biodegradable materials include polyglactin, polyglycolic acid, autogenous, heterogenous, and xenogeneic tissue (e.g., pericardium or small intestine submucosa), and oxidized, regenerated cellulose. These meshes can be knitted, woven or non-woven meshes. Examples of non-woven meshes include electrospun materials.

Meshes and films may be prepared from non-biodegradable polymers. Representative examples of non-biodegradable compositions include ethylene-co-vinyl acetate copolymers, acrylic-based and methacrylic-based polymers (e.g., poly(acrylic acid), poly(methylacrylic acid), poly(methylmethacrylate), poly(hydroxyethylmethacrylate), poly(alkylcynoacrylate), poly(alkyl acrylates), poly(alkyl methacrylates)), polyolefins such as poly(ethylene) or poly(propylene), polyamides (e.g., nylon 6,6), poly(urethanes) (e.g., poly(ester urethanes), poly(ether urethanes), poly(carbonate urethanes), poly(ester-urea)), polyesters (e.g., PET, polybutyleneterephthalate, and polyhexyleneterephthalate), polyethers (poly(ethylene oxide), poly(propylene oxide), poly(ethylene oxide)-poly(propylene oxide) copolymers, diblock and triblock copolymers, poly(tetramethylene glycol)), silicone containing polymers and vinyl-based polymers (polyvinylpyrrolidone, poly(vinyl alcohol), poly(vinyl acetate phthalate), poly(styrene-co-isobutylene-co-styrene), fluorine containing polymers (fluoropolymers) such as fluorinated ethylene propylene (FEP) or polytetrafluoroethylene (e.g., expanded PTFE).

The mesh or film material may comprise a combination of the above-mentioned biodegradable and non-degradable polymers. Further examples of polymers that may be used are either anionic (e.g., alginate, carrageenin, hyaluronic acid, dextran sulfate, chondroitin sulfate, carboxymethyl dextran, caboxymethyl cellulose and poly(acrylic acid)], or cationic [e.g., chitosan, poly-1-lysine, polyethylenimine, and poly(allyl amine)) (see generally, Dunn et al., J. Applied Polymer Sci. 50: 353, 1993; Cascone et al., J. Materials Sci.: Materials in Medicine 5: 770, 1994; Shiraishi et al., Biol. Pharm. Bull. 16: 1164, 1993; Thacharodi and Rao, Int'l J. Pharm. 120: 115, 1995; Miyazaki et al., Int'l J. Pharm. 118: 257, 1995). Preferred polymers (including copolymers and blends of these polymers) include poly(ethylene-co-vinyl acetate), poly(carbonate urethanes), poly(hydroxyl acids) (e.g., poly(D,L-lactic acid) oligomers and polymers, poly(L-lactic acid) oligomers and polymers, poly(D-lactic acid) oligomers and polymers, poly(glycolic acid), copolymers of lactic acid and glycolic acid, copolymers of lactide and glycolide, poly(caprolactone), copolymers of lactide or glycolide and ε-caprolactone), poly(valerolactone), poly(anhydrides), copolymers prepared from caprolactone and/or lactide and/or glycolide and/or polyethylene glycol.

A variety of polymeric and non-polymeric films and meshes have been described which may be combined with an anti-scarring agent. For example, the film or mesh may be a biodegradable polymeric matrix that conforms to the tissue and releases the agent in a controlled release manner. See, e.g., U.S. Pat. No. 6,461,640. The film or mesh may be a self-adhering silicone sheet which is impregnated with an antioxidant and/or antimicrobial. See, e.g., U.S. Pat. No. 6,572,878. The film or mesh may be a pliable shield with attachment ports and fenestrations that is adapted to cover a bony dissection in the spine. See, e.g., U.S. Pat. No. 5,868,745 and U.S. Patent Application No. 2003/0078588. The film or mesh may be a resorbable micro-membrane having a single layer of non-porous polymer base material of poly-lactide. See, e.g., U.S. Pat. No. 6,531,146 and U.S. Application No. 2004/0137033. The film or mesh may be a flexible neuro decompression device that has an outer surface texturized with microstructures to reduce fibroplasia when it is wrapped around a nerve in a canal. See, e.g., U.S. Pat. No. 6,106,558. The film or mesh may be a resorbable collagen membrane that is wrapped around the spinal chord to inhibit cell adhesions. See, e.g., U.S. Pat. No. 6,221,109. The film or mesh may be a wound dressing garment composed of an outer pliable layer and a self-adhesive inner gel lining which serves as a dressing for contacting wounds. See, e.g., U.S. Pat. No. 6,548,728. The film or mesh may be a bandage with a scar treatment pad with a layer of silicone elastomer or silicone gel. See, e.g., U.S. Pat. Nos. 6,284,941 and 5,891,076. The film or mesh may be a crosslinkable system with at least three reactive compounds each having a polymeric molecular core with at least one functional group. See, e.g., U.S. Pat. No. 6,458,889. The film or mesh may be composed of a prosthetic fabric having a 3-dimensional structure separating two surfaces in which one is open to post-surgical cell colonization and one is linked to a film of collagenous material. See, e.g., U.S. Pat. No. 6,451,032. The film or mesh may be composed by crosslinking two synthetic polymers, one having nucleophilic groups and the other having electrophilic groups, such that they form a matrix that may be used to incorporate a biologically active compound. See, e.g., U.S. Pat. Nos. 6,323,278; 6,166,130; 6,051,648 and 5,874,500. The film or mesh may be a film composed of hetero-bifunctional anti-adhesion binding agents that act to covalently link substrate materials, such as collagen, to receptive tissue. See, e.g., U.S. Pat. No. 5,580,923. The film or mesh may be a conformable warp-knit fabric of oxidized regenerated cellulose or other bioresorbable material which acts like a physical barrier to prevent postoperative adhesions. See, e.g., U.S. Pat. No. 5,007,916. Meshes for use in the practice of the invention also are described in U.S. Pat. No. 6,575,887, and co-pending application, entitled “Perivascular Wraps,” filed Sep. 26, 2003 (U.S. Ser. No. (U.S. Ser. No. 10/673,046).

In one aspect, the mesh may be suitable for use in hernia repair surgery or in other types of surgical procedures. Mesh fabrics for use in connection with hernia repairs are disclosed in U.S. Pat. Nos. 6,638,284; 5,292,328; 4,769,038 and 2,671,444. Surgical meshes may be produced by knitting, weaving, braiding, or otherwise forming a plurality of yarns (e.g., monofilament or multifilament yarns made of polymeric materials such as polypropylene and polyester) into a support trellis. Knitted and woven fabrics constructed from a variety of synthetic fibers and the use of the fabrics, in surgical repair are also discussed in U.S. Pat. Nos. 3,054,406; 3,124,136; 4,193,137; 4,347,847; 4,452,245; 4,520,821; 4,633,873; 4,652,264; 4,655,221; 4,838,884 and 5,002,551 and European Patent Application No. 334,046. Implantable hernia meshes are described in U.S. Pat. Nos. 6,610,006; 6,368,541 and 6,319,264. Hernia meshes for the repair of hiatal hernias are described in, e.g., U.S. Pat. No. 6,436,030. Hernia meshes for the repair of abdominal (e.g., ventral and umbilical) hernias are described in U.S. Pat. No. 6,383,201. Infection-resistant hernia meshes are described in, e.g., U.S. Pat. No. 6,375,662. Hernia meshes such as those described in the patents listed above are suitable for combining with a fibrosis-inducing agent to create a mesh which promotes the growth of fibrous tissue.

In one aspect, the fibrosis-inhibiting agent can be incorporated into a biodegradable or dissolvable film or mesh that is then applied to the treatment site prior or post implantation of the prosthesis/implant. Exemplary materials for the manufacture of these films or meshes are hyaluronic acid (crosslinked or non-crosslinked), cellulose derivatives (e.g., hydroxypropyl cellulose), PLGA, collagen and crosslinked poly(ethylene glycol).

The film or mesh may be in the form of a tissue graft, which may be an autograft, allograft, biograft, biogenic graft or xenograft. Tissue grafts may be derived from various tissue types. Representative examples of tissues that may be used to prepare biografts include, but are not limited to, rectus sheaths, peritoneum, bladder, pericardium, veins, arteries, diaphragm and pleura. The biograft may be harvested from a host, loaded with an anti-scarring agent and then applied in a perivascular manner at the site where lesions and intimal hyperplasia can develop (e.g., at an anastomotic site). Once implanted, the agent (e.g., paclitaxel) is released from the graft and can penetrate the vessel wall to prevent the formation of intimal hyperplasia at the treatment site. In certain embodiments, the biograft may be used as a backing layer to enclose a composition (e.g., a gel or paste loaded with anti-scarring agent).

Films and meshes, which may be combined with one or more anti-scarring agents according to the present invention, include commercially available products. Examples of films and meshes into which a fibrosis agent can be incorporated include INTERCEED (Johnson & Johnson, Inc.), PRECLUDE (W.L. Gore), and POLYACTIVE (poly(ether ester) multiblock copolymers (Osteotech, Inc., Shrewsbury, N.J.), based on poly(ethylene glycol) and poly(butylene terephthalate), and SURGICAL absorbable hemostat gauze-like sheet from Johnson & Johnson. Another mesh is a prosthetic polypropylene mesh with a bioresorbable coating called SEPRAMESH Biosurgical Composite (Genzyme Corporation, Cambridge, Mass.). One side of the mesh is coated with a bioresorbable layer of sodium hyaluronate and carboxymethylcellulose, providing a temporary physical barrier that separates the underlying tissue and organ surfaces from the mesh. The other side of the mesh is uncoated, allowing for complete tissue ingrowth similar to bare polypropylene mesh. In one embodiment, the fibrosis-inducing agent may be applied only to the uncoated side of SEPRAMESH and not to the sodium hyaluronate/carboxymethylcellulose coated side. Other films and meshes include: (a) BARD MARLEX mesh (C.R. Bard, Inc.), which is a very dense knitted fabric structure with low porosity; (b) monofilament polypropylene mesh such as PROLENE available from Ethicon, Inc. Somerville, N.J. (see, e.g., U.S. Pat. Nos. 5,634,931 and 5,824,082)); (c) SURGISIS GOLD and SURGISIS IHM soft tissue graft (both from Cook Surgical, Inc.) which are devices specifically configured for use to reinforce soft tissue in repair of inguinal hernias in open and laparoscopic procedures; (d) thin walled polypropylene surgical meshes such as are available from Atrium Medical Corporation (Hudson, N.H.) under the trade names PROLITE, PROLITE ULTRA, and LITEMESH; (e) COMPOSIX hernia mesh (C.R. Bard, Murray Hill, N.J.), which incorporates a mesh patch (the patch includes two layers of an inert synthetic mesh, generally made of polypropylene, and is described in U.S. Pat. No. 6,280,453) that includes a filament to stiffen and maintain the device in a flat configuration; (f) VISILEX mesh (from C.R. Bard, Inc.), which is a polypropylene mesh that is constructed with monofilament polypropylene; (g) other meshes available from C.R. Bard, Inc. which include PERFIX Plug, KUGEL Hernia Patch, 3D MAX mesh, LHI mesh, DULEX mesh, and the VENTRALEX Hernia Patch; and (h) other types of polypropylene monofilament hernia mesh and plug products include HERTRA mesh 1, 2, and 2A, HERMESH 3, 4 & 5 and HERNIAMESH plugs T1, T2, and T3 from Herniamesh USA, Inc. (Great Neck, N.Y.).

Other examples of commercially available meshes which may be combined with fibrosis-inhibiting agents are described below. One example includes a prosthetic polypropylene mesh with a bioresorbable coating sold under the trade name SEPRAMESH Biosurgical Composite (Genzyme Corporation). One side of the mesh is coated with a bioresorbable layer of sodium hyaluronate and carboxymethylcellulose, providing a temporary physical barrier that separates the underlying tissue and organ surfaces from the mesh. The other side of the mesh is uncoated, allowing for complete tissue ingrowth similar to bare polypropylene mesh. In one embodiment, the fibrosis-inducing agent may be applied only to the uncoated side of SEPRAMESH and not to the sodium hyaluronate/carboxymethylcellulose coated side. Boston Scientific Corporation sells the TRELEX NATURAL Mesh which is composed of a unique knitted polypropylene material. Ethicon, Inc. makes the absorbable VICRYL (polyglactin 910) meshes (knitted and woven) and MERSILENE Polyester Fiber Mesh. Dow Corning Corporation (Midland, Mich.) sells a mesh material formed from silicone elastomer known as SILASTIC Rx Medical Grade Sheeting (Platinum Cured). United States Surgical/Syneture (Norwalk, Conn.) sells a mesh made from absorbable polyglycolic acid under the trade name DEXON Mesh Products. Membrana Accurel Systems (Obernburg, Germany) sells the CELGARD microporous polypropylene fiber and membrane. Gynecare Worldwide, a division of Ethicon, Inc. sells a mesh material made from oxidized, regenerated cellulose known as INTERCEED TC7. Integra LifeSciences Corporation (Plainsboro, N.J.) makes DURAGEN PLUS Adhesion Barrier Matrix, which can be used as a barrier against adhesions following spinal and cranial surgery and for restoration of the dura mater. HYDROSORB Shield from MacroPore Biosurgery, Inc. (San Diego, Calif.) is a film for temporary wound support to control the formation of adhesions in specific spinal applications.

Numerous polymeric and non-polymeric carrier systems that can be used with films and meshes have been described above. Methods for incorporating the fibrosis-inhibiting compositions onto or into the film or mesh include: (a) affixing (directly or indirectly) to the film or mesh a fibrosis-inhibiting composition (e.g., by either a spraying process or dipping process as described above, with or without a carrier), (b) incorporating or impregnating into the film or mesh a fibrosis-inhibiting composition (e.g., by either a spraying process or dipping process as described above, with or without a carrier (c) by coating the film or mesh with a substance such as a hydrogel which will in turn absorb the fibrosis-inhibiting composition, (d) constructing the film or mesh itself or a portion of the film or mesh with a fibrosis-inhibiting composition, or (e) by covalently binding the fibrosis-inhibiting agent directly to the film or mesh surface or to a linker (small molecule or polymer) that is coated or attached to the film or mesh surface. For devices that are coated, the coating process can be performed in such a manner as to (a) coat only one surface of the film or mesh or (b) coat all or parts of both sides of the film or mesh.

The therapeutic agent(s) may be an integral part of the film or mesh (i.e., may reside within the fibers of the mesh). The fibrosis inhibiting agent can be incorporated directly into the film or mesh or it can be incorporated into a secondary carrier (polymeric or non-polymeric), as described above, that is then incorporated into the film or mesh.

The film or mesh may be coated with a fibrosis-inhibiting agent or a composition that includes the fibrosis-inhibiting agent. In some embodiments, the composition is a polymer composition can function as a surgical adhesion barrier. The coating may take the form of a surface-adherent coating, mask, film, gel, foam, or mold.

A variety of polymeric compositions have been described that may be used in conjunction with the films and meshes of the invention. Such compositions may be in the form of, for example, gels, sprays, liquids, and pastes, or may be polymerized from monomeric or prepolymeric constituents in situ. For example, the composition may be a polymeric tissue coating which is formed by applying a polymerization initiator to the tissue and then covering it with a water-soluble macromer that is polymerizable using free radical initiators under the influence of UV light. See, e.g., U.S. Pat. Nos. 6,177,095 and 6,083,524. The composition may be an aqueous composition including a surfactant, pentoxifylline and a polyoxyalkylene polyether. See, e.g., U.S. Pat. No. 6,399,624. The composition may be a hydrogel-forming, self-solvating, absorbable polyester copolymers capable of selective, segmental association into compliant hydrogels mass upon contact with an aqueous environment. See, e.g., U.S. Pat. No. 5,612,052. The composition may be composed of fluent pre-polymeric material that is emitted to the tissue surface and then exposed to activating energy in situ to initiate conversion of the applied material to non-fluent polymeric form. See, e.g., U.S. Pat. Nos. 6,004,547 and 5,612,050. The composition may be composed of a gas mixture of oxygen present in a volume ratio of 1 to 20%. See, e.g., U.S. Pat. No. 6,428,500. The composition may be composed of an anionic polymer having an acid sulfate and sulfur content greater than 5% which acts to inhibit monocyte or macrophage invasion. See, e.g., U.S. Pat. No. 6,417,173. The composition may be composed of a non-gelling polyoxyalkylene composition with or without a therapeutic agent. See, e.g., U.S. Pat. No. 6,436,425. The composition may be coated onto tissue surfaces and may be composed of an aqueous solution of a hydrophilic, polymeric material (e.g., polypeptides or polysaccharide) having greater than 50,000 molecular weight and a concentration range of 0.01% to 15% by weight. See, e.g., U.S. Pat. No. 6,464,970.

Other representative examples of polymeric compositions which may be coated onto the film or mesh include poly(ethylene glycol)-based systems, hyaluronic acid and crosslinked hyaluronic acid compositions. These compositions can be applied as the final composition or they can be applied as materials that form crosslinked gel in situ.

Other compositions that can be used in conjunction with films and meshes, include, but are not limited to: (a) sprayable PEG-containing formulations such as COSEAL, SPRAYGEL, FOCALSEAL or DURASEAL; (b) hyaluronic acid-containing formulations such as RESTYLANE, HYLAFORM, PERLANE, SYNVISC, SEPRAFILM, SEPRACOAT, INTERGEL, (c) polymeric gels such as REPEL or FLOWGEL, (d) dextran sulfate gels such as the ADCON range of products, (e) lipid based compositions such as ADSURF (Brittania Pharmaceuticals).

The film or mesh (or device comprising the film or mesh) may be made sterile either by preparing them under aseptic environment and/or they may be terminally sterilized using methods known in the art, such as gamma radiation or electron beam sterilization methods or a combination of both of these methods.

Films and meshes may be applied to any bodily conduit or any tissue that may be prone to the development of fibrosis or intimal hyperplasia. Prior to implantation, the film or mesh may be trimmed or cut from a sheet of bulk material to match the configuration of the widened foramen, canal, or dissection region, or at a minimum, to overlay the exposed tissue area. The film or mesh may be bent or shaped to match the particular configuration of the placement region. The film or mesh may also be rolled in a cuff shape or cylindrical shape and placed around the exterior periphery of the desired tissue. The film or mesh may be provided in a relatively large bulk sheet and then cut into shapes to mold the particular structure and surface topography of the tissue or device to be wrapped. Alternatively, the film or mesh may be pre-shaped into one or more patterns for subsequent use. The films and meshes may be typically rectangular in shape and be placed at the desired location within the surgical site by direct surgical placement or by endoscopic techniques. The film or mesh may be secured into place by wrapping it onto itself (i.e., self-adhesive), or by securing it with sutures, staples, sealant, and the like. Alternatively, the film or mesh may adhere readily to tissue and therefore, additional securing mechanisms may not be required.

The films or meshes of the invention may be used for a variety of indications, including, without limitation: (a) prevention of surgical adhesions between tissues following surgery (e.g., gyneacologic surgery, vasovasostomy, hernia repair, nerve root decompression surgery and laminectomy); (b) prevention of hypertrophic scars or keloids (e.g., resulting from tissue burns or other wounds); (c) prevention of intimal hyperplasia and/or restenosis (e.g., resulting from insertion of vascular grafts or hemodialysis access devices); or (d) may be used in affiliation with devices and implants that lead to scarring as described herein (e.g., as a sleeve or mesh around a breast implant to reduce or inhibit scarring).

In one embodiment, films or meshes may be used to prevent adhesions that occur between tissues following surgery, injury or disease. Adhesion formation, a complex process in which bodily tissues that are normally separate grow together, occurs most commonly as a result of surgical intervention and/or trauma. Generally, adhesion formation is an inflammatory reaction in which factors are released, increasing vascular permeability and resulting in fibrinogen influx and fibrin deposition. This deposition forms a matrix that bridges the abutting tissues. Fibroblasts accumulate, attach to the matrix, deposit collagen and induce angiogenesis. If this cascade of events can be prevented within 4 to 5 days following surgery, then adhesion formation can be inhibited. Adhesion formation or unwanted scar tissue accumulation and encapsulation complicates a variety of surgical procedures and virtually any open or endoscopic surgical procedure in the abdominal or pelvic cavity. Encapsulation of surgical implants also complicates breast reconstruction surgery, joint replacement surgery, hernia repair surgery, artificial vascular graft surgery, and neurosurgery. In each case, the implant becomes encapsulated by a fibrous connective tissue capsule which compromises or impairs the function of the surgical implant (e.g., breast implant, artificial joint, surgical mesh, vascular graft, dural patch). Chronic inflammation and scarring also occurs during surgery to correct chronic sinusitis or removal of other regions of chronic inflammation (e.g., foreign bodies, infections (fungal, mycobacterium). Surgical procedures that may lead to surgical adhesions may include cardiac, spinal, neurologic, pleural, thoracic and gynaecologic surgeries. However, adhesions may also develop as a result of other processes, including, but not limited to, non-surgical mechanical injury, ischemia, hemorrhage, radiation treatment, infection-related inflammation, pelvic inflammatory disease and/or foreign body reaction. This abnormal scarring interferes with normal physiological functioning and, in come cases, can force and/or interfere with follow-up, corrective or other surgical operations. For example, these post-operative surgical adhesions occur in 60 to 90% of patients undergoing major gynaecologic surgery and represent one of the most common causes of intestinal obstruction in the industrialized world. These adhesions are a major cause of failed surgical therapy and are the leading cause of bowel obstruction and infertility. Other adhesion-treated complications include chronic pelvic pain, urethral obstruction and voiding dysfunction.

Currently, preventative therapies, administered 4 to 5 days following surgery, are used to inhibit adhesion formation. Various modes of adhesion prevention have been examined, including (1) prevention of fibrin deposition, (2) reduction of local tissue inflammation, and (3) removal of fibrin deposits. Fibrin deposition is prevented through the use of physical adhesion barriers that are either mechanical or comprised of viscous solutions. Although many investigators are utilizing adhesion prevention barriers, a number of technical difficulties exist.

In one aspect, the present invention provides films and meshes that include an anti-scarring agent or a composition that includes an anti-scarring agent for use as surgical adhesion barriers.

In one aspect, films and meshes may be used to prevent surgical adhesions in the epidural and dural tissue which is a factor contributing to failed back surgeries and complications associated with spinal injuries (e.g., compression and crush injuries). Scar formation within dura and around nerve roots has been implicated in rendering subsequent spine operations technically more difficult. To gain access to the spinal foramen during back surgeries, vertebral bone tissue is often disrupted. Back surgeries, such as laminectomies and diskectomies, often leave the spinal dura exposed and unprotected. As a result, scar tissue frequently forms between the dura and the surrounding tissue. This scar is formed from the damaged erector spinae muscles that overlay the laminectomy site. This results in adhesion development between the muscle tissue and the fragile dura, thereby, reducing mobility of the spine and nerve roots which leads to pain and slow post-operative recovery. To circumvent adhesion development, a scar-reducing barrier may be inserted between the dural sleeve and the paravertebral musculature post-laminotomy. This reduces cellular and vascular invasion into the epidural space from the overlying muscle and exposed cancellous bone and thus, reduces the complications associated with the canal housing the spinal chord and/or nerve roots.

In another aspect, films and meshes comprising an anti-scarring agent may be used to prevent the fibrosis from occurring between a hernia repair mesh and the surrounding tissue. Hernias are abnormal protrusions (outpouchings) of an organ or other body structure through a defect or natural opening in a covering membrane, muscle or bone. Hernias themselves are not dangerous, but can become extremely problematic if they become incarcerated. Surgical prostheses used in hernia repair (referred to herein as “hernia meshes”) include prosthetic mesh- or gauze-like materials, which support the repaired hernia or other body structures during the healing process. Hernias are often repaired surgically to prevent complications. Conditions in which a hernia mesh may need to be used include, without limitation, the repair of inguinal (i.e., groin), umbilical, ventral, femoral, abdominal, diaphragmatic, epigastric, gastroesophageal, hiatal, intermuscular, mesenteric, paraperitoneal, rectovaginal, rectocecal, uterine, and vesical hernias. Hernia repair typically involves returning the viscera to its normal location and the defect in the wall is primarily closed with sutures, but for bigger gaps, a mesh is placed over the defect to close the hernia opening. Inclusion of an anti-scarring agent or composition comprising an anti-scarring agent into or onto a hernia repair mesh may reduce or prevent fibrosis proximate to the implanted hernia mesh, thereby minimizing the possibility of adhesions between the abdominal wall or other tissues and the mesh itself, and reducing further complications and abdominal pain.

In yet another aspect, films or meshes may be used to prevent hypertrophic scars or keloids (e.g., resulting from tissue burns or other wounds). Hypertrophic scars and keloids are the result of an excessive fibroproliferative wound healing response. Briefly, healing of wounds and scar formation occurs in three phases: inflammation, proliferation, and maturation. The first phase, inflammation, occurs in response to an injury which is severe enough to break the skin. During this phase, which lasts 3 to 4 days, blood and tissue fluid form an adhesive coagulum and fibrinous network which serves to bind the wound surfaces together. This is then followed by a proliferative phase in which there is ingrowth of capillaries and connective tissue from the wound edges, and closure of the skin defect. Finally, once capillary and fibroblastic proliferation has ceased, the maturation process begins wherein the scar contracts and becomes less cellular, less vascular, and appears flat and white. This final phase may take between 6 and 12 months. If too much connective tissue is produced and the wound remains persistently cellular, the scar may become red and raised. If the scar remains within the boundaries of the original wound it is referred to as a hypertrophic scar, but if it extends beyond the original scar and into the surrounding tissue, the lesion is referred to as a keloid. Hypertrophic scars and keloids are produced during the second and third phases of scar formation. Several wounds are particularly prone to excessive endothelial and fibroblastic proliferation, including burns, open wounds, and infected wounds. With hypertrophic scars, some degree of maturation occurs and gradual improvement occurs. In the case of keloids however, an actual tumor is produced which can become quite large. Spontaneous improvement in such cases rarely occurs. A film or mesh that comprises an anti-scarring agent or a composition that comprises an anti-scarring agent may be placed in contact with a wound or burn site in order to prevent formation of hypertrophic scar or keloids.

In yet another aspect, films and meshes are provided that may be used for delivering an anti-scarring agent to an external portion (surface) of a body passageway or cavity. Examples of body passageways include arteries, veins, the heart, the esophagus, the stomach, the duodenum, the small intestine, the large intestine, biliary tracts, the ureter, the bladder, the urethra, lacrimal ducts, the trachea, bronchi, bronchiole, nasal airways, eustachian tubes, the external auditory mayal, vas deferens and fallopian tubes. Examples of cavities include the abdominal cavity, the buccal cavity, the peritoneal cavity, the pericardial cavity, the pelvic cavity, perivisceral cavity, pleural cavity and uterine cavity.

Examples of conditions that may be treated or prevented with fibrosis-inhibiting films and meshes include iatrogenic complications of arterial and venous catheterization, complications of vascular dissection, complications of gastrointestinal passageway rupture and dissection, restonotic complications associated with vascular surgery (e.g., bypass surgery), and intimal hyperplasia.

In one aspect, an anti-scarring agent may be delivered from a film or mesh to the external walls of body passageways or cavities for the purpose of preventing and/or reducing a proliferative biological response that may obstruct or hinder the optimal functioning of the passageway or cavity, including, for example, iatrogenic complications of arterial and venous catheterization, aortic dissection, cardiac rupture, aneurysm, cardiac valve dehiscence, graft placement (e.g., A-V-bypass, peripheral bypass, CABG), fistula formation, passageway rupture and surgical wound repair.

The films or meshes may be used in the form of a perivascular wrap to prevent restenosis at anastomotic sites resulting from insertion of vascular grafts or hemodialysis access devices. In this case, perivascular wraps may be incorporated with or coated with a fibrosis-inhibiting agent, which can be used in conjunction with a vascular graft to inhibit scarring at an anastomotic site. These films or meshes may be placed or wrapped in a perivascular (periadventitial) manner around the outside of the anastomosis at the time of surgery. Film and mesh implants comprising an anti-scarring agent may be used with synthetic bypass grafts (femoral-popliteal, femoral-femoral, axillary-femoral etc.), vein grafts (peripheral and coronary), internal mammary (coronary) grafts or hemodialysis grafts (AV fistulas, AV access grafts).

In order to further the understanding of such conditions, representative complications leading to compromised body passageway or cavity integrity are discussed in more detail below.

Cardiac Bypass Surgery

Coronary artery bypass graft (“CABG”) surgery was introduced in the 1950s, and still remains a highly invasive, open surgical procedure, although less invasive surgical techniques are being developed. CABG surgery is a surgical procedure that is performed to overcome many types of coronary artery blockages. The purpose of bypass surgery is to increase the circulation and nourishment to the heart muscle that has been reduced due to arterial blockage. This procedure involves the surgeon accessing the heart and the diseased arteries, usually through an incision in the middle of the chest. Often, healthy arteries or veins are “harvested” from the patient to create “bypass grafts” that channel the needed blood flow around the blocked portions of the coronary arteries. The arteries or veins are connected from the aorta to the surface of the heart beyond the blockages thereby forming an autologous graft. This allows the blood to flow through these grafts and “bypass” the narrowed or closed vessel. The use of synthetic graft materials to create the “bypass” has been limited due to the lack of the appropriate biocompatibility of these synthetic grafts. CABG has significant short term limitations, including medical complications, such as stroke, multiple organ dysfunction, inflammatory response, respiratory failure and post-operative bleeding, each of which may result in death. Another problem associated with CABG is restenosis. Restenosis is typically defined as a renarrowing of an arterial blood vessel within six months of the CABG procedure. It typically occurs in approximately 25% to 45% of patients, and is the result of an excessive healing response to arterial injury after a revascularization procedure. Restenosis may occur within a short period following a procedure or may develop over the course of months or years. Longer term or “late” restenosis may result from excessive proliferation of scar tissue at the treatment site, the causes of which are not well understood. Thus any product that may reduce the incidence or magnitude of the restenotic process following CABG surgery can greatly enhance the well being of a patient.

In order to prevent the restenotic complications associated with CABG surgery, such as those discussed above, a wide variety of therapeutic agents (with or without a carrier) may be delivered to the external portion of the blood vessel. The carrier (e.g., polymer) or therapeutic agent/polymer composition can be applied to the external portion of the vessel following the interventional or surgical procedure in order to prevent the restenotic complications.

Peripheral Bypass Surgery

Peripheral arterial disease (PAD) refers to diseases of any of the blood vessels outside of the heart. PAD is a range of disorders that may affect the blood vessels in the hands, arms, legs, or feet. The most common form of PAD is atherosclerosis. Atherosclerosis is a gradual process in which cholesterol and scar tissue build up in the arteries to form plaque. This build-up causes a gradual narrowing of the artery, which leads to a decrease in the amount of blood flow through that artery. When the flow of blood decreases, it results in a decrease of oxygen and nutrient supply to the body's tissues, which in turn may result in pain sensation. When the arteries to the legs are affected, the most common symptom is pain in the calf when walking. This is known as intermittent claudication.

Peripheral bypass surgery is a procedure to bypass an area of stenosed (narrowed) or blocked artery that is a result of atherosclerosis. In this surgical procedure, a synthetic graft (artificial blood vessels) or an autologous graft, vein, will be implanted to provide blood flow around the diseased area. First, the surgeon makes an incision in the leg, thigh, calf or ankle skin. The location of the incision may vary based on which vessels need to be bypassed and where there is healthy artery to connect to maintain the blood flow. The bypass graft is sewn into the artery above the stenosis or blockage, and below the stenosis or blockage. This bypass provides a means whereby blood will reach the tissue that has not been receiving enough blood and oxygen. Synthetic bypass grafts used in the legs are usually made of ePTFE.

Restenosis and occlusion of bypass grafts are one of the most important problems in peripheral bypass surgery. This restenosis is caused by neointimal growth (hyperplasia) and is especially pronounced within artificial graft material. This restenosis is usually at the anastomotic site where the graft and artery are connected via a surgical procedure. The intimal tissue typically grows from the native vessel into the graft. In order to prevent the restenotic complications associated with peripheral bypass surgery, such as those discussed above, a wide variety of therapeutic agents (with or without a carrier)/polymer compositions may be delivered to the external portion of the blood vessel. The polymer or therapeutic agent/polymer composition can be applied to the external portion of the vessel/anastomotic site following the interventional or surgical procedure in order to prevent the restenotic complications.

Arterio-Venous (AV) Fistula

The arterio-venous (AV) fistula is surgically created vascular connection which allows the flow of blood from an artery directly to a vein. The AV fistula was first created by researchers for kidney failure patients who must undergo kidney dialysis.

Hemodialysis requires a viable artery and vein to draw blood from and return it to the body. The repeated puncturing often either causes a vein or artery to fail or causes other complications for the patient. The AV fistula increases the amount of possible puncture sites for hemodialysis and minimizes the damage to the patient's natural blood vessels. The connection that is created between the vein and artery forms a large blood vessel that continuously supplies an increased blood flow for performing hemodialysis.

Restenosis and eventual occlusion are one of the most important problems in the long term patency of the AV fistula. In order to prevent the restenotic complications associated with the surgical formation of an AV fistula, a wide variety of therapeutic agents (with or without a carrier)/polymer compositions may be delivered to the external portion of the blood vessel. The polymer or therapeutic agent/polymer composition can be applied to the external portion of the vessel/anastomotic site following the interventional or surgical procedure in order to prevent the restenotic complications.

Arterio-Venous (AV) Graft Surgery

The AV graft surgical procedure is used for similar application as those for the AV fistula (e.g., hemodialysis patients). For the AV graft surgery, a synthetic graft material is used to connect the artery to the vein rather that the direct connection of the artery to the vein as is the case for the AV fistula. The incidence of intimal hyperplasia, which leads to occlusion of the graft, is one of the main factors that affect the long term patency of these grafts. This intimal hyperplasia may occur at the venous anastomosis and at the floor of the vein. A product that may reduce or prevent this occurrence of intimal hyperplasia will increase the duration of patency of these grafts. In order to reduce the occurrence of intimal hyperplasia at the venous anastomosis of an AV graft, a wide variety of therapeutic agents (with or without a carrier)/polymer compositions may be delivered to the external portion of the blood vessel. The polymer or therapeutic agent/polymer composition can be applied to the external portion of the vessel/anastomotic site following the interventional or surgical procedure in order to prevent the restenotic complications.

Anastomotic Closure Devices

Anastomotic closure devices provide a means for rapidly repairing an anastomosis. The use of some of these devices requires an invasive surgical procedure. In one embodiment of this invention, following the use of an anastomotic closure device, the mesh containing the therapeutic agent may be wrapped around the anastomosis and the anastomotic closure device, if it is left at the surgical site.

In one embodiment, the invention provides a method for treating or preventing intimal hyperplasia that includes delivering to an anastomotic site a delivery device. The device includes a therapeutic agent and a biodegradable polymer, wherein at least some of the biodegradable polymer is in the form of a mesh. Exemplary anastomotic sites include venous anastomosis, arterial anastomosis, arteriovenous fistula, arterial bypass, and arteriovenous graft. Preferably, the device includes a polymer mesh with a therapeutic agent is delivered to an external portion of an anastomotic site.

Transplant Applications

There are many applications in which various organs in the human body fail to function in a manner to sustain the well being of the patient. When an appropriate donor organ is available, an impaired organ may be replaced by a donor organ (e.g., lung, heart, kidney etc). One of the potential complications following these transplant surgeries is the potential for stenosis to occur in the blood vessels at or near the anastomotic site between the donor and recipient vessels. For example, transplant renal artery stenosis is a complication that may occur following a kidney transplant. Transplant renal artery stenosis is when the artery from the abdominal aorta to the kidney narrows, limiting blood flow to the kidney. This may also make it difficult to keep blood pressure under control. Treatment typically involves expanding the narrowed segment using a small balloon.

One method to treat this stenosis is to apply the composition of this invention around the anastomotic site (junction of the donor and recipient vessels) in a perivascular manner. In a similar manner, the composition of this invention may be applied in a peritubular manner to the exterior surfaces of the trachea and or bronchi following a lung transplant procedure.

According to the present invention, any scarring agent described above can be utilized in the practice of this embodiment. Films and meshes may be adapted to contain and/or release an agent that inhibits one or more of the four general components of the process of fibrosis (or scarring), including: formation of new blood vessels (angiogenesis), migration and proliferation of connective tissue cells (such as fibroblasts or smooth muscle cells), deposition of extracellular matrix (ECM), and remodeling (maturation and organization of the fibrous tissue).

As films and meshes are made in a variety of configurations and sizes, the exact dose administered will vary with device size, surface area and design. However, certain principles can be applied in the application of this art. Drug dose can be calculated as a function of dose per unit area (of the portion of the device being coated), total dose administered, and appropriate surface concentrations of active drug can be determined. Drugs are to be used at concentrations that range from several times more than to 10%, 5%, or even less than 1% of the concentration typically used in a single chemotherapeutic systemic dose application. Preferably, the drug is released in effective concentrations for a period ranging from 1-90 days.

Several examples of fibrosis-inhibiting agents for use in films or meshes include the following: cell cycle inhibitors including (A) anthracyclines (e.g., doxorubicin and mitoxantrone), (B) taxanes (e.g., paclitaxel, TAXOTERE and docetaxel), and (C) podophyllotoxins (e.g., etoposide); (D) immunomodulators (e.g., sirolimus, everolimus, tacrolimus); (E) heat shock protein 90 antagonists (e.g., geldanamycin); (F) HMGCoA reductase inhibitors (e.g., simvastatin); (G) inosine monophosphate dehydrogenase inhibitors (e.g., mycophenolic acid, 1-alpha-25 dihydroxy vitamin D₃); (H)NF kappa B inhibitors (e.g., Bay 11-7082); (I) antimycotic agents (e.g., sulconizole), (J) p38 MAP kinase inhibitors (e.g., SB202190), and (K) and anti-angiogenesis agents (e.g., halofuginone bromide), as well as analogues and derivatives of the aforementioned.

Regardless of the method of application of the drug to the film or mesh, the exemplary anti-fibrosing agents, used alone or in combination, should be administered under the following dosing guidelines. The total amount (dose) of anti-scarring agent in or on the film or mesh may be in the range of about 0.01 μg-10 μg, or 10 μg-10 mg, or 10 mg-250 mg, or 250 mg-1000 mg, or 1000 mg-2500 mg. The dose (amount) of anti-scarring agent per unit area of device surface to which the agent is applied may be in the range of about 0.01 μg/mm²-1 μg/mm², or 1 μg/mm²-10 μg/mm², or 10 μg/mm²-250 μg/mm², 250 μg/mm²-1000 μg/mm², or 1000 μg/mm²-2500 μg/mm².

Provided below are exemplary dosage ranges for various anti-scarring agents that can be used in conjunction with films or meshes in accordance with the invention. A) Cell cycle inhibitors including doxorubicin and mitoxantrone. Doxorubicin analogues and derivatives thereof: total dose not to exceed 25 mg (range of 0.1 μg to 25 mg); preferred 1 μg to 5 mg. The dose per unit area of 0.01 μg-100 μg per mm²; preferred dose of 0.1 μg/mm²-10 μg/mm². Minimum concentration of 10⁻⁸-10⁻⁴ M of doxorubicin is to be maintained on the device surface. Mitoxantrone and analogues and derivatives thereof: total dose not to exceed 5 mg (range of 0.01 μg to 5 mg); preferred 0.1 μg to 1 mg. The dose per unit area of the device of 0.01 μg-20 μg per mm²; preferred dose of 0.05 μg/mm²-3 μg/mm². Minimum concentration of 10⁻⁸-10⁻⁴M of mitoxantrone is to be maintained on the device surface. B) Cell cycle inhibitors including Paclitaxel and analogues and derivatives (e.g., docetaxel) thereof: total dose not to exceed 10 mg (range of 0.1 μg to 10 mg); preferred 1 μg to 3 mg. The dose per unit area of the device of 0.1 μg-10 μg per mm²; preferred dose of 0.25 μg/mm²-5 μg/mm². Minimum concentration of 10⁻⁸-10⁻⁴ M of paclitaxel is to be maintained on the device surface. (C) Cell cycle inhibitors such as podophyllotoxins (e.g., etoposide): total dose not to exceed 10 mg (range of 0.1 μg to 10 mg); preferred 1 μg to 3 mg. The dose per unit area of the device of 0.1 μg-10 μg per mm²; preferred dose of 0.25 μg/mm²-5 μg/mm². Minimum concentration of 10⁻⁸-10⁻⁴ M of etoposide is to be maintained on the device surface. (D) Immunomodulators including sirolimus and everolimus. Sirolimus (i.e., rapamycin, RAPAMUNE): Total dose not to exceed 10 mg (range of 0.1 μg to 10 mg); preferred 10 μg to 1 mg. The dose per unit area of 0.1 μg-100 μg per mm²; preferred dose of 0.5 μg/mm²-10 μg/mm². Minimum concentration of 10⁻⁸-10⁻⁴ M is to be maintained on the device surface. Everolimus and derivatives and analogues thereof: Total dose should not exceed 10 mg (range of 0.1 μg to 10 mg); preferred 10 μg to 1 mg. The dose per unit area of 0.1 μg-100 μg per mm² of surface area; preferred dose of 0.3 μg/mm²-10 μg/mm². Minimum concentration of 10⁻⁸-10⁻⁴ M of everolimus is to be maintained on the device surface. (E) Heat shock protein 90 antagonists (e.g., geldanamycin) and analogues and derivatives thereof: total dose not to exceed 20 mg (range of 0.1 μg to 20 mg); preferred 1 μg to 5 mg. The dose per unit area of the device of 0.1 μg-10 μg per mm²; preferred dose of 0.25 μg/mm²-5 μg/mm². Minimum concentration of 10⁻⁸-10⁻⁴ M of geldanamycin is to be maintained on the device surface. (F) HMGCoA reductase inhibitors (e.g., simvastatin) and analogues and derivatives thereof: total dose not to exceed 2000 mg (range of 10.0 μg to 2000 mg); preferred 10 μg to 300 mg. The dose per unit area of the device of 1.0 μg-1000 μg per mm²; preferred dose of 2.5 μg/mm²-500 μg/mm². Minimum concentration of 10⁻⁸-10⁻³ M of simvastatin is to be maintained on the device surface. (G) Inosine monophosphate dehydrogenase inhibitors (e.g., mycophenolic acid, 1-alpha-25 dihydroxy vitamin D₃) and analogues and derivatives thereof: total dose not to exceed 2000 mg (range of 10.0 μg to 2000 mg); preferred 10 μg to 300 mg. The dose per unit area of the device of 1.0 μg-1000 μg per mm²; preferred dose of 2.5 μg/mm²-500 μg/mm². Minimum concentration of 10⁻⁸-10⁻³ M of mycophenolic acid is to be maintained on the device surface. (H)NF kappa B inhibitors (e.g., Bay 11-7082) and analogues and derivatives thereof: total dose not to exceed 200 mg (range of 1.0 μg to 200 mg); preferred 1 μg to 50 mg. The dose per unit area of the device of 1.0 μg-100 μg per mm²; preferred dose of 2.5 μg/mm²-50 μg/mm². Minimum concentration of 10⁻⁸-10⁴ M of Bay 11-7082 is to be maintained on the device surface. (I) Antimycotic agents (e.g., sulconizole) and analogues and derivatives thereof: total dose not to exceed 2000 mg (range of 10.0 μg to 2000 mg); preferred 10 μg to 300 mg. The dose per unit area of the device of 1.0 μg-1000 μg per mm²; preferred dose of 2.5 μg/mm²-500 μg/mm². Minimum concentration of 10⁻⁸-10⁻³ M of sulconizole is to be maintained on the device surface. (J) p38 MAP kinase inhibitors (e.g., SB202190) and analogues and derivatives thereof: total dose not to exceed 2000 mg (range of 10.0 μg to 2000 mg); preferred 10 μg to 300 mg. The dose per unit area of the device of 1.0 μg-1000 μg per mm²; preferred dose of 2.5 μg/mm²-500 μg/mm². Minimum concentration of 10⁻⁸-10⁻³ M of SB202190 is to be maintained on the device surface. (K) Anti-angiogenic agents (e.g., halofuginone bromide) and analogues and derivatives thereof: total dose not to exceed 10 mg (range of 0.1 μg to 10 mg); preferred 1 μg to 3 mg. The dose per unit area of the device of 0.1 μg-10 μg per mm²; preferred dose of 0.25 μg/mm²-5 μg/mm². Minimum concentration of 10⁻⁸-10⁻⁴ M of halofuginone bromide is to be maintained on the device surface.

In addition to those described above (e.g., sirolimus, everolimus, and tacrolimus), several other examples of immunomodulators and appropriate dosages ranges for use with meshes and films include the following: (A) Biolimus and derivatives and analogues thereof: Total dose should not exceed 10 mg (range of 0.1 μg to 10 mg); preferred 10 μg to 1 mg. The dose per unit area of 0.1 μg-100 μg per mm² of surface area; preferred dose of 0.3 μg/mm²-10 μg/mm². Minimum concentration of 10⁻⁸-10⁻⁴ M of everolimus is to be maintained on the device surface. (B) Tresperimus and derivatives and analogues thereof: Total dose should not exceed 10 mg (range of 0.1 μg to 10 mg); preferred 10 μg to 1 mg. The dose per unit area of 0.1 μg-100 μg per mm² of surface area; preferred dose of 0.3 μg/mm²-10 μg/mm². Minimum concentration of 10⁻⁸-10⁻⁴ M of tresperimus is to be maintained on the device surface. (C) Auranofin and derivatives and analogues thereof: Total dose should not exceed 10 mg (range of 0.1 μg to 10 mg); preferred 10 μg to 1 mg. The dose per unit area of 0.1 μg-100 μg per mm² of surface area; preferred dose of 0.3 μg/mm²-10 μg/mm². Minimum concentration of 10⁻⁸-10⁻⁴ M of auranofin is to be maintained on the device surface. (D) 27-0-Demethylrapamycin and derivatives and analogues thereof: Total dose should not exceed 10 mg (range of 0.1 μg to 10 mg); preferred 10 μg to 1 mg. The dose per unit area of 0.1 μg-100 μg per mm² of surface area; preferred dose of 0.3 μg/mm²-10 μg/mm². Minimum concentration of 10⁻⁸-10⁻⁴ M of 27-0-Demethylrapamycin is to be maintained on the device surface. (E) Gusperimus and derivatives and analogues thereof: Total dose should not exceed 10 mg (range of 0.1 μg to 10 mg); preferred 10 μg to 1 mg. The dose per unit area of 0.1 μg-100 μg per mm² of surface area; preferred dose of 0.3 μg/mm²-10 μg/mm². Minimum concentration of 10⁻⁸-10⁻⁴ M of gusperimus is to be maintained on the device surface. (F) Pimecrolimus and derivatives and analogues thereof: Total dose should not exceed 10 mg (range of 0.1 μg to 10 mg); preferred 10 μg to 1 mg. The dose per unit area of 0.1 μg-100 μg per mm² of surface area; preferred dose of 0.3 μg/mm²-10 μg/mm². Minimum concentration of 10⁻⁸-10⁻⁴ M of pimecrolimus is to be maintained on the device surface and (G) ABT-578 and analogues and derivatives thereof: Total dose should not exceed 10 mg (range of 0.1 μg to 10 mg); preferred 10 μg to 1 mg. The dose per unit area of 0.1 μg-100 μg per mm² of surface area; preferred dose of 0.3 μg/mm²-10 μg/mm². Minimum concentration of 10⁻⁸-10⁻⁴ M of ABT-578 is to be maintained on the device surface.

In addition to those described above (e.g., paclitaxel, TAXOTERE, and docetaxel), several other examples of anti-microtubule agents and appropriate dosages ranges for use with meshes and films include vinca alkaloids such as vinblastine and vincristine sulfate and analogues and derivatives thereof: total dose not to exceed 10 mg (range of 0.1 μg to 10 mg); preferred 1 μg to 3 mg. Dose per unit area of the device of 0.1 μg-10 μg per mm²; preferred dose of 0.25 μg/mm²-5 μg/mm². Minimum concentration of 10⁻⁸-10⁻⁴ M of drug is to be maintained on the device surface.

Glaucoma Drainage Devices

In one aspect, the present invention provides for the combination of an anti-scarring agent and a glaucoma drainage device.

Various types of glaucoma drainage devices have been described. Some glaucoma drainage devices include a plate and a tube. The function of the tube is to deliver aqueous from within the eye onto the upper surface of the episcleral plate. The episcleral plate is firmly sutured to the sclera and covered by a thick flap of Tenon's tissue and conjunctiva. The function of the plate is to initiate the formation of a large circular bleb which develops a specialized fibrovascular bleb lining and becomes distended by aqueous. It is this fibrovascular bleb lining which is responsible for regulating the escape of aqueous from the eye and which determines the final level of intraocular pressure (IOP) that is achieved after insertion of the implant. If the fibrovascular response is too great, the drainage capability of the device is reduced. In an embodiment of the present invention, a fibrosis-inhibiting agent is incorporated into or onto all or a portion of the device such that the released fibrosis-inhibiting agent modulates the healing response, thereby enabling the device to function correctly.

Glaucoma drainage devices may be, for example, a conduit attached to an episcleral drainage plate having a porous posterior surface for cellular ingrowth and attachment by the sclera. See, e.g., U.S. Pat. No. 5,882,327. The glaucoma drainage device may be composed of a foldable and rollable episcleral plate and a drainage tube whereby the device may be delivered to the implant site through an injection delivery system. See, e.g., U.S. Pat. No. 6,589,203. The glaucoma drainage device may be pressure regulator composed of a base plate formed of a thin, flexible rubber material (e.g., silicone rubber) which has a mounted housing chamber that is attached to a tube. See, e.g., U.S. Pat. No. 5,752,928. The glaucoma drainage device may be composed of an elastomeric plate having a sealing member that conforms to the sclera to restrict fluid and an attached non-valved elastomeric drainage tube. See, e.g., U.S. Pat. No. 5,476,445. The glaucoma drainage device may be composed of ridged plates that extend outwardly that are concave on one side to match the curvature of the sclera and are adapted for side by side attachment to the sclera whereby a tube extends between the ridged plates for communication. See, e.g., U.S. Pat. No. 4,457,757. The glaucoma drainage device may be composed of a thin, elliptical, elastomeric plate having a centrally positioned hole for growth of scar tissue and an elastomeric drainage tube attached to the plate for fluid communication with the eye. See, e.g., U.S. Pat. No. 5,397,300. The glaucoma drainage device may be composed of a tube with a circumferential hole with a connected disk at the outlet end of the tube for placing on a surface of an eyeball. See, e.g., U.S. Pat. No. 5,868,697. The glaucoma drainage device may be a tube with a flow controlling structure that constricts flow passage within the tube and has at least one circumferential hole within the tube that is temporarily occluded with an absorbable material. See, e.g., U.S. Pat. No. 6,203,513. The glaucoma drainage device may be composed of a tube with an engagement means and a porous, liquid-absorbing plug with an attached filamentary extension that substantially restricts fluid flow. See, e.g., U.S. Pat. No. 5,300,020. The glaucoma drainage device may be a resilient polymeric drain implant with a passage extending between the ends and flanges that project radially from the body. See, e.g., U.S. Pat. No. 4,968,296. The glaucoma drainage device may be a shunt to divert aqueous humor in the eye from the anterior chamber into a portion of the device that branches to provide fluid communication in either direction along the Schlemm's canal. See, e.g., U.S. Pat. No. 6,626,858.

Glaucoma drainage devices, which may be combined with one or more anti-scarring agents according to the present invention, include commercially available products. For example, cylindrical tubes, such as the AQUAFLOW Collagen Glaucoma Drainage Device (STAAR Surgical Company, Monrovia, Calif.) may be used in the practice of the present invention. Other examples of glaucoma drainage devices includes the Molteno Glaucoma Implant (Single Plate Molteno Implant, Pressure Ridge Single Plate Molteno Implant (D1), Microphthalmic Plate Molteno Implant (M1), Double Plate Molteno Implant (R2/L2), and Pressure Ridge Double Plate Molteno Implant (DR2/DL2) from Molteno Opthalmic Limited (New Zealand), BAERVELDT Glaucoma Implants (Models BG-101-350, BG-102-350, BG-103-250; Pfizer, New York, N.Y.), and the Ahmed Glaucoma Valve (Models FP7, S2, S3, PS2, PS3, B1 from New World Medical, Inc. (Rancho Cucamonga, Calif.).

In one aspect, the present invention provides a glaucoma drainage device that includes an anti-scarring agent or a composition that includes an anti-scarring agent. Numerous polymeric and non-polymeric delivery systems for use in glaucoma drainage devices have been described above. Methods for incorporating the fibrosis-inhibiting agent into or onto the device includes: (a) directly affixing to the device a fibrosis-inhibiting composition (e.g., by either a spraying process or dipping process as described above, with or without a carrier),
(b) directly incorporating into the device a fibrosis-inhibiting composition (e.g., by either a spraying process or dipping process as described above, with or without a carrier), (c) by coating the device with a substance such as a hydrogel which will in turn absorb the fibrosis-inhibiting composition, (d) by interweaving fibrosis-inhibiting composition coated thread (or the polymer itself formed into a thread) into the device structure, (e) by inserting the device into a sleeve or mesh which is comprised of or coated with a fibrosis-inhibiting composition, (f) constructing the device itself or a portion of the device with a fibrosis-inhibiting composition, or (g) by covalently binding the fibrosis-inhibiting agent directly to the device surface or to a linker (small molecule or polymer) that is coated or attached to the device surface.

In addition to coating the device with the fibrosis-inhibiting composition, the fibrosis-inhibiting agent can be mixed with the materials that are used to make the device such that the fibrosis-inhibiting agent is incorporated into the final device.

In one embodiment, the methods above can be used to incorporate the fibrosis-inhibiting agent into or onto all or portions of the plate of the device.

In another embodiment, the methods above can be used to incorporate the fibrosis-inhibiting agent into or onto all or portions of the tube of the device.

In yet another embodiment, the methods above can be used to incorporate the fibrosis-inhibiting agent into or onto all or potions of both the plate and the tube of the device.

In addition to incorporation of a fibrosis-inhibiting agent into or onto the device (e.g., as a coating), another biologically active agent can be incorporated into or onto the device, for example an anti-inflammatory (e.g., dexamethazone or aspirin) or a MMP inhibitor.

According to the present invention, any fibrosis-inhibiting agent described above can be utilized in the practice of this embodiment. Within one embodiment of the invention, glaucoma drainage devices may be adapted to release an agent that inhibits one or more of the four general components of the process of fibrosis (or scarring), including: formation of new blood vessels (angiogenesis), migration and proliferation of connective tissue cells (such as fibroblasts or smooth muscle cells), deposition of extracellular matrix (ECM), and remodeling (maturation and organization of the fibrous tissue). By inhibiting one or more of the components of fibrosis (or scarring), the overgrowth of granulation tissue may be inhibited or reduced.

As glaucoma drainage devices are made in a variety of configurations and sizes, the exact dose administered will vary with device size, surface area and design. However, certain principles can be applied in the application of this art. Drug dose can be calculated as a function of dose per unit area (of the portion of the device being coated), total dose administered, and appropriate surface concentrations of active drug can be determined. Drugs are to be used at concentrations that range from several times more than to 10%, 5%, or even less than 1% of the concentration typically used in a single chemotherapeutic systemic dose application. Preferably, the drug is released in effective concentrations for a period ranging from 1-90 days.

Several examples of fibrosis-inhibiting agents for use in glaucoma drainage devices include the following: cell cycle inhibitors including (A) anthracyclines (e.g., doxorubicin and mitoxantrone), (B) taxanes (e.g., paclitaxel, TAXOTERE and docetaxel), and (C) podophyllotoxins (e.g., etoposide); (D) immunomodulators (e.g., sirolimus, everolimus, tacrolimus); (E) heat shock protein 90 antagonists (e.g., geldanamycin); (F) HMGCoA reductase inhibitors (e.g., simvastatin); (G) inosine monophosphate dehydrogenase inhibitors (e.g., mycophenolic acid, 1-alpha-25 dihydroxy vitamin D₃); (H)NF kappa B inhibitors (e.g., Bay 11-7082); (I) antimycotic agents (e.g., sulconizole), (J) p38 MAP kinase inhibitors (e.g., SB202190), and (K) and anti-angiogenesis agents (e.g., halofuginone bromide), as well as analogues and derivatives of the aforementioned.

Regardless of the method of application of the drug to the devices, the exemplary anti-fibrosing agents, used alone or in combination, should be administered under the following dosing guidelines. The total amount (dose) of anti-scarring agent in or on the device may be in the range of about 0.01 μg-10 μg, or 10 μg-10 mg, or 10 mg-250 mg, or 250 mg-1000 mg, or 1000 mg-2500 mg. The dose (amount) of anti-scarring agent per unit area of device surface to which the agent is applied may be in the range of about 0.01 μg/mm²-1 μg/mm², or 1 μg/mm²-10 μg/mm², or 10 μg/mm²-250 μg/mm², 250 μg/mm²-1000 μg/mm², or 1000 μg/mm²-2500 μg/mm².

Provided below are exemplary dosage ranges for various anti-scarring agents that can be used in conjunction with glaucoma drainage devices in accordance with the invention. A) Cell cycle inhibitors including doxorubicin and mitoxantrone. Doxorubicin analogues and derivatives thereof: total dose not to exceed 25 mg (range of 0.1 μg to 25 mg); preferred 1 μg to 5 mg. The dose per unit area of 0.01 μg-100 μg per mm²; preferred dose of 0.1 μg/mm²-10 μg/mm². Minimum concentration of 10⁻⁸-10⁻⁴ M of doxorubicin is to be maintained on the device surface. Mitoxantrone and analogues and derivatives thereof: total dose not to exceed 5 mg (range of 0.01 μg to 5 mg); preferred 0.1 μg to 1 mg. The dose per unit area of the device of 0.01 μg-20 μg per mm²; preferred dose of 0.05 μg/mm²-3 μg/mm². Minimum concentration of 10⁻⁸-10⁻⁴ M of mitoxantrone is to be maintained on the device surface. B) Cell cycle inhibitors including Paclitaxel and analogues and derivatives (e.g., docetaxel) thereof: total dose not to exceed 10 mg (range of 0.1 μg to 10 mg); preferred 1 μg to 3 mg. The dose per unit area of the device of 0.1 μg-10 μg per mm²; preferred dose of 0.25 μg/mm²-5 μg/mm². Minimum concentration of 10⁻⁸-10⁻⁴ M of paclitaxel is to be maintained on the device surface. (C) Cell cycle inhibitors such as podophyllotoxins (e.g., etoposide): total dose not to exceed 10 mg (range of 0.1 μg to 10 mg); preferred 1 μg to 3 mg. The dose per unit area of the device of 0.1 μg-10 μg per mm²; preferred dose of 0.25 μg/mm²-5 μg/mm². Minimum concentration of 10⁻⁸-10⁻⁴ M of etoposide is to be maintained on the device surface. (D) Immunomodulators including sirolimus and everolimus. Sirolimus (i.e., rapamycin, RAPAMUNE): Total dose not to exceed 10 mg (range of 0.1 μg to 10 mg); preferred 10 μg to 1 mg. The dose per unit area of 0.1 μg-100 μg per mm²; preferred dose of 0.5 μg/mm²-10 μg/mm². Minimum concentration of 10⁻⁸-10⁴ M is to be maintained on the device surface. Everolimus and derivatives and analogues thereof: Total dose should not exceed 10 mg (range of 0.1 μg to 10 mg); preferred 10 μg to 1 mg. The dose per unit area of 0.1 μg-100 μg per mm² of surface area; preferred dose of 0.3 μg/mm²-10 μg/mm². Minimum concentration of 10⁻⁸-10⁻⁴ M of everolimus is to be maintained on the device surface. (E) Heat shock protein 90 antagonists (e.g., geldanamycin) and analogues and derivatives thereof: total dose not to exceed 20 mg (range of 0.1 μg to 20 mg); preferred 1 μg to 5 mg. The dose per unit area of the device of 0.1 μg-10 μg per mm²; preferred dose of 0.25 μg/mm²-5 μg/mm². Minimum concentration of 10⁻⁸-10⁻⁴ M of geldanamycin is to be maintained on the device surface. (F) HMGCoA reductase inhibitors (e.g., simvastatin) and analogues and derivatives thereof: total dose not to exceed 2000 μg (range of 10.0 μg to 2000 mg); preferred 10 μg to 300 mg. The dose per unit area of the device of 1.0 μg-1000 μg per mm²; preferred dose of 2.5 μg/mm²-500 μg/mm². Minimum concentration of 10⁻⁸-10⁻³ M of simvastatin is to be maintained on the device surface. (G) Inosine monophosphate dehydrogenase inhibitors (e.g., mycophenolic acid, 1-alpha-25 dihydroxy vitamin D₃) and analogues and derivatives thereof: total dose not to exceed 2000 mg (range of 10.0 μg to 2000 mg); preferred 10 μg to 300 mg. The dose per unit area of the device of 1.0 μg-1000 μg per mm²; preferred dose of 2.5 μg/mm²-500 μg/mm². Minimum concentration of 10⁻⁸-10⁻³ M of mycophenolic acid is to be maintained on the device surface. (H)NF kappa B inhibitors (e.g., Bay 11-7082) and analogues and derivatives thereof: total dose not to exceed 200 mg (range of 1.0 μg to 200 mg); preferred 1 μg to 50 mg. The dose per unit area of the device of 1.0 μg-100 μg per mm²; preferred dose of 2.5 μg/mm²-50 μg/mm². Minimum concentration of 10⁻⁸-10⁻⁴ M of Bay 11-7082 is to be maintained on the device surface. (I) Antimycotic agents (e.g., sulconizole) and analogues and derivatives thereof: total dose not to exceed 2000 mg (range of 10.0 μg to 2000 mg); preferred 10 μg to 300 mg. The dose per unit area of the device of 1.0 μg-1000 μg per mm²; preferred dose of 2.5 μg/mm²-500 μg/mm². Minimum concentration of 10⁻⁸-10⁻³ M of sulconizole is to be maintained on the device surface. (J) p38 MAP kinase inhibitors (e.g., SB202190) and analogues and derivatives thereof: total dose not to exceed 2000 mg (range of 10.0 μg to 2000 mg); preferred 10 μg to 300 mg. The dose per unit area of the device of 1.0 μg-1000 μg per mm²; preferred dose of 2.5 μg/mm²-500 μg/mm². Minimum concentration of 10⁻⁸-10⁻³ M of SB202190 is to be maintained on the device surface. (K) Anti-angiogenic agents (e.g., halofuginone bromide) and analogues and derivatives thereof: total dose not to exceed 10 mg (range of 0.1 μg to 10 mg); preferred 1 μg to 3 mg. The dose per unit area of the device of 0.1 μg-10 μg per mm²; preferred dose of 0.25 μg/mm²-5 μg/mm². Minimum concentration of 10⁻⁸-10⁻⁴ M of halofuginone bromide is to be maintained on the device surface.

In addition to those described above (e.g., sirolimus, everolimus, and tacrolimus), several other examples of immunomodulators and appropriate dosages ranges for use with glaucoma drainage devices include the following: (A) Biolimus and derivatives and analogues thereof: Total dose should not exceed 10 mg (range of 0.1 μg to 10 mg); preferred 10 μg to 1 mg. The dose per unit area of 0.1 μg-100 μg per mm² of surface area; preferred dose of 0.3 μg/mm²-10 μg/mm². Minimum concentration of 10⁻⁸-10⁻⁴ M of everolimus is to be maintained on the device surface. (B) Tresperimus and derivatives and analogues thereof: Total dose should not exceed 10 mg (range of 0.1 μg to 10 mg); preferred 10 μg to 1 mg. The dose per unit area of 0.1 μg-100 μg per mm² of surface area; preferred dose of 0.3 μg/mm²-10 μg/mm². Minimum concentration of 10⁻⁸-10⁻⁴ M of tresperimus is to be maintained on the device surface. (C) Auranofin and derivatives and analogues thereof: Total dose should not exceed 10 mg (range of 0.1 μg to 10 mg); preferred 10 μg to 1 mg. The dose per unit area of 0.1 μg-100 μg per mm of surface area; preferred dose of 0.3 μg/mm²-10 μg/mm². Minimum concentration of 10⁻⁸-10⁻⁴ M of auranofin is to be maintained on the device surface. (D) 27-0-Demethylrapamycin and derivatives and analogues thereof: Total dose should not exceed 10 mg (range of 0.1 μg to 10 mg); preferred 10 μg to 1 mg. The dose per unit area of 0.1 μg-100 μg per mm² of surface area; preferred dose of 0.3 μg/mm²-10 μg/mm². Minimum concentration of 10⁻⁸-10⁻⁴ M of 27-0-Demethylrapamycin is to be maintained on the device surface. (E) Gusperimus and derivatives and analogues thereof: Total dose should not exceed 10 mg (range of 0.1 μg to 10 mg); preferred 10 μg to 1 mg. The dose per unit area of 0.1 μg-100 μg per mm² of surface area; preferred dose of 0.3 μg/mm²-10 μg/mm². Minimum concentration of 10⁻¹⁸-10⁻⁴ M of gusperimus is to be maintained on the device surface. (F) Pimecrolimus and derivatives and analogues thereof: Total dose should not exceed 10 mg (range of 0.1 μg to 10 mg); preferred 10 μg to 1 mg. The dose per unit area of 0.1 μg-100 μg per mm² of surface area; preferred dose of 0.3 μg/mm²-10 μg/mm². Minimum concentration of 10⁻⁸-10⁻⁴ M of pimecrolimus is to be maintained on the device surface and (G) ABT-578 and analogues and derivatives thereof: Total dose should not exceed 10 mg (range of 0.1 μg to 10 mg); preferred 10 μg to 1 mg. The dose per unit area of 0.1 μg-100 μg per mm² of surface area; preferred dose of 0.3 μg/mm²-10 μg/mm². Minimum concentration of 10⁻⁸-10⁻⁴ M of ABT-578 is to be maintained on the device surface.

In addition to those described above (e.g., paclitaxel, TAXOTERE, and docetaxel), several other examples of anti-microtubule agents and appropriate dosages ranges for use with glaucoma drainage devices include vinca alkaloids such as vinblastine and vincristine sulfate and analogues and derivatives thereof: total dose not to exceed 10 mg (range of 0.1 μg to 10 mg); preferred 1 μg to 3 mg. Dose per unit area of the device of 0.1 μg-10 μg per mm²; preferred dose of 0.25 μg/mm²-5 μg/mm². Minimum concentration of 10⁻⁸-10⁻⁴ M of drug is to be maintained on the device surface.

Prosthetic Heart Valves

The present invention provides for the combination of a drug and a prosthetic heart valve.

Prosthetic heart valves are devices that are used to replace natural heart valves that are defective, due to congenital malformations, infections, partial occlusion, or wearing. Prosthetic heart valves are typically composed of an occluder(s) attached to the occluder base, which is in turn attached to the suture ring that provides anchorage of the device to the heart tissue. The occluder base is annular and provides a passageway for blood flow. There may be one or more occluders which alternate in an opened and closed position to regulate the flow of blood. To secure the prosthetic heart valve to the heart tissue, a suture ring, typically composed of a knit fabric tube, is rolled into a toroidal form and is secured to the periphery of the occluder base of the prosthesis. Affixing the suture ring to the heart tissue typically occurs using sutures, sealants, adhesives, staples, or clamping with metal or polymer wires.

Although the design of prosthetic heart valves has been gradually refined, complications continue to occur. Since the suture rings are often made out of synthetic material, thrombus, fibrosis and pannus often occur around the prosthetic heart valve. This scar formation often hinders the function of the valve and over time may require a second surgical procedure and replacement. Suture rings are generally composed of synthetic polymer, including, but not limited to, polyester (e.g., DACRON), polytetrafluoroethylene (e.g., TEFLON), silicone, and polypropylene. Suture rings are often made of a filler material with a woven material stitched over the filler. The surface of the suture ring is often course due to the covering cloth material. This predisposes the suture ring to scarring formation early in the post-operative period with severe pannus/fibrosis developing over several months following implantation. The consequences of fibrosis encroachment onto a prosthetic heart valve can be drastic, and potentially catastrophic. For example, fibrosis may inhibit valve occluder function by limiting its ability to open and close properly. The fibrosis may extend from the suture ring to the leaflets. This fibrosis may fuse the leaflets at their commissure, distort individual leaflets, and/or stiffen leaflets such that they do not open or close properly. The end result of this fibrosis typically is a heart valve that is both stenotic and insufficient.

There are two main types of prosthetic heart valves, mechanical and bioprosthetic. Typically, both mechanical and bioprosthetic heart valves utilize a synthetic suture ring. They differ primarily in the type of occluder that is utilized. The occluders of the mechanical heart valve may be composed of a ball and cage assembly, single leaflet disk valves, or bileaflet disk valves. The occluders of the bioprosthetic heart valve are composed of animal or human tissue that mimic the appearance and function of the natural heart valve it is replacing. The bioprosthetic heart valve leaflets are usually composed of chemically treated tissue. The harvested valves are fixed in glutaraldehyde or similar fixatives in order to make them suitable for human implantation.

In one aspect, the prosthetic heart valve may be a mechanical prosthesis which is typically composed of rigid leaflets formed of a biocompatible substance (e.g., pyrolitic carbon, titanium or DACRON). Mechanical prosthetic heart valves may be a ball and cage assembly, bileaflet, trileaflet or tilting disks. The most common is the bileaflet type since the hemodynamics of this valve is better as blood flow is smoother and less turbulent. For example, the mechanical prosthesis may be composed of a base with an external suture ring and an internal rim for blood flow as well as at least two closing leaflets. See, e.g., U.S. Pat. No. 6,068,657. The mechanical prosthesis may be composed of annular valve housing with a center orifice and first and second valve leaflets pivotally mounted to the valve housing. See, e.g., U.S. Pat. Nos. 4,808,180 and 5,026,391. The mechanical prosthesis may be designed with an annular body with at least one leaflet pivotally mounted such that it is movable between an open and closed position by a magnet that exerts a force on the leaflet at a defined pressure. See, e.g., U.S. Pat. No. 6,638,303. The mechanical prosthesis may have an annular body with a plurality of hinges which form an entrance ramp and supports at least one leaflet to the valve body. See, e.g., U.S. Pat. Nos. 6,645,244 and 5,919,226. The mechanical prosthesis may be composed of a supporting flexible, cylindrical frame with a cover that forms a cusp supporting stent for the valve trileaflet apparatus and a sewing ring as an attachment surface. See, e.g., U.S. Pat. No. 5,258,023. The mechanical prosthesis may have an increased valve lumen composed of a single piece valve orifice housing with at least one movable occluder coupled to the housing and a suture cuff for attaching the housing to the heart tissue. See, e.g., U.S. Pat. Nos. 6,007,577 and 6,391,053. The mechanical prosthesis may be composed of a sewing ring and a removable valve assembly which slides in a central core of the sewing ring. See, e.g., U.S. Pat. No. 5,032,128. The mechanical prosthesis may be a highly flexible cylindrical stent composed of a plurality of separate adjacent stent members with alternating cusps and commissures that are able to move radially and support a plurality of flexible leaflets. See, e.g., U.S. Pat. Nos. 6,558,418 and 6,338,740. Other mechanical heart valve prostheses are described in, e.g., U.S. Pat. Nos. 6,395,025; 6,358,278; 6,176,877; 6,139,575 and 5,984,958.

In another aspect, the prosthetic heart valve may be a bioprosthetic device which typically is flexible leaflets formed of a biological material (e.g., porcine valves or bovine pericardial valves). Tissue valves may be supported with a stent frame that provides the leaflets with more structure and durability. Stentless tissue valves may also be implanted by harvesting the porcine valves with the pig's aorta still attached. For example, the bioprosthetic heart valve, which may be obtained from a donor (e.g., porcine), may be treated to reduce antigens to prevent inflammatory response upon transplantation. See, e.g., U.S. Pat. No. 6,592,618. The bioprosthetic heart valve may be composed of a biological tissue material disposed around a mechanical annular support to provide at least part of the sewing ring. See, e.g., U.S. Pat. No. 6,582,464. The bioprosthetic heart valve may be composed of a xenograft mitral valve (e.g., porcine) and a sewing tube and cover of flexible material which is attached to the mitral valve. See, e.g., U.S. Pat. No. 5,662,704. The bioprosthetic heart valve may be composed of a natural tissue heart valve attached to a prosthetic stent frame that may be covered by a fabric cover. See, e.g., U.S. Pat. Nos. 3,983,581; 4,035,849; 5,861,028; 6,350,282 and 6,585,766. The bioprosthetic heart valve may be a self-supporting stentless valve that may be composed of a tubular body of mammalian origin. See, e.g., U.S. Pat. Nos. 5,156,621 and 6,342,070.

In another aspect, the prosthetic heart valve may be inserted into place using minimally-invasive techniques. For example, the prosthetic heart valve may be an expandable device adapted for delivery in a collapsed state to an implantation site and then expanded to a plurality of leaflets attached to a stent system. See, e.g., U.S. Pat. No. 6,454,799.

In another aspect, the device may be a component of the heart valve. For example, the device may be an implantable annular ring for receiving a prosthetic heart valve. See, e.g., U.S. Pat. No. 6,106,550. The device may be a suture ring having an outer peripheral tapered thread for attaching a heart valve prosthesis. See, e.g., U.S. Pat. No. 6,113,632. The device may be a suture ring for a mechanical heart valve composed of a stiffening ring attachment, a knit fabric sewing cuff and a locking ring. See, e.g., U.S. Pat. No. 5,071,431.

Prosthetic heart valves and components thereof (e.g., annular suture rings), which may be combined with one or more drugs according to the present invention, include commercially available products, such as the Carpentier-Edwards PERIMOUNT (CEP) Pericardial Bioprosthesis, Carpentier-Edwards S.A.V. Aortic Bioprosthesis and Edwards PRIMA PLUS STENTLESS BIOPROSTHESIS from Edwards Lifesciences (Irvine, Calif.), the SJM REGENT Valve from St. Jude Medical (St. Paul, Minn.), and the MOSAIC Bioprosthetic Heart Valve from Medtronic (Minneapolis, Minn.).

In one aspect, the present invention provides prosthetic heart valve devices that include an anti-scarring agent or a composition that includes an anti-scarring agent. Numerous polymeric and non-polymeric delivery systems for use in prosthetic heart valves have been described above. Methods for incorporating the fibrosis-inhibiting agent into or onto the device includes: (a) directly affixing to the device a fibrosis-inhibiting composition (e.g., by either a spraying process or dipping process as described above, with or without a carrier), (b) directly incorporating into the device a fibrosis-inhibiting composition (e.g., by either a spraying process or dipping process as described above, with or without a carrier (c) by coating the device with a substance such as a hydrogel which will in turn absorb the fibrosis-inhibiting composition, (d) by interweaving fibrosis-inhibiting composition coated thread (or the polymer itself formed into a thread) into the device structure, (e) constructing the device itself or a portion of the device with a fibrosis-inhibiting composition, or (f) by covalently binding the fibrosis-inhibiting agent directly to the device surface or to a linker (small molecule or polymer) that is coated or attached to the device surface, and/or (g) any combination of the aforementioned.

According to the present invention, any fibrosis-inhibiting agent described above can be utilized in the practice of this embodiment. Within one embodiment of the invention, prosthetic heart valves may be adapted to release an agent that inhibits one or more of the four general components of the process of fibrosis (or scarring), including: formation of new blood vessels (angiogenesis), migration and proliferation of connective tissue cells (such as fibroblasts or smooth muscle cells), deposition of extracellular matrix (ECM), and remodeling (maturation and organization of the fibrous tissue). By inhibiting one or more of the components of fibrosis (or scarring), the overgrowth of granulation tissue may be inhibited or reduced.

As prosthetic heart valve devices are made in a variety of configurations and sizes, the exact dose administered will vary with device size, surface area and design. However, certain principles can be applied in the application of this art. Drug dose can be calculated as a function of dose per unit area (of the portion of the device being coated), total dose administered, and appropriate surface concentrations of active drug can be determined. Drugs are to be used at concentrations that range from several times more than to 10%, 5%, or even less than 1% of the concentration typically used in a single chemotherapeutic systemic dose application. Preferably, the drug is released in effective concentrations for a period ranging from 1-90 days.

Several examples offibrosis-inhibiting agents for use in prosthetic heart valves include the following: cell cycle inhibitors including (A) anthracyclines (e.g., doxorubicin and mitoxantrone), (B) taxanes (e.g., paclitaxel, TAXOTERE and docetaxel), and (C) podophyllotoxins (e.g., etoposide); (D) immunomodulators (e.g., sirolimus, everolimus, tacrolimus); (E) heat shock protein 90 antagonists (e.g., geldanamycin); (F) HMGCoA reductase inhibitors (e.g., simvastatin); (G) inosine monophosphate dehydrogenase inhibitors (e.g., mycophenolic acid, 1-alpha-25 dihydroxy vitamin D₃); (H)NF kappa B inhibitors (e.g., Bay 11-7082); (I) antimycotic agents (e.g., sulconizole), (J) p38 MAP kinase inhibitors (e.g., SB202190), and (K) and anti-angiogenesis agents (e.g., halofuginone bromide), as well as analogues and derivatives of the aforementioned.

Regardless of the method of application of the drug to the prosthetic heart valve, the exemplary anti-fibrosing agents, used alone or in combination, should be administered under the following dosing guidelines. The total amount (dose) of anti-scarring agent in or on the prosthetic heart valve may be in the range of about 0.01 μg-10 μg, or 10 μg-10 mg, or 10 mg-250 mg, or 250 mg-1000 mg, or 1000 mg-2500 mg. The dose (amount) of anti-scarring agent per unit area of device surface to which the agent is applied may be in the range of about 0.01 μg/mm²-1 μg/mm², or 1 μg/mm²-10 μg/mm², or 10 μg/mm²-250 μg/mm², 250 μg/mm²-1000 μg/mm², or 1000 μg/mm²-2500 μg/mm².

Provided below are exemplary dosage ranges for various anti-scarring agents that can be used in conjunction with prosthetic heart valve devices in accordance with the invention. A) Cell cycle inhibitors including doxorubicin and mitoxantrone. Doxorubicin analogues and derivatives thereof: total dose not to exceed 25 mg (range of 0.1 μg to 25 mg); preferred 1 μg to 5 mg. The dose per unit area of 0.01 μg-100 μg per mm²; preferred dose of 0.1 μg/mm²-10 μg/mm². Minimum concentration of 10⁻⁸-10⁻⁴ M of doxorubicin is to be maintained on the device surface. Mitoxantrone and analogues and derivatives thereof: total dose not to exceed 5 mg (range of 0.01 μg to 5 mg); preferred 0.1 μg to 1 mg. The dose per unit area of the device of 0.01 μg-20 μg per mm²; preferred dose of 0.05 μg/mm²-3 μg/mm². Minimum concentration of 10⁻⁸-10⁻⁴ M of mitoxantrone is to be maintained on the device surface. B) Cell cycle inhibitors including Paclitaxel and analogues and derivatives (e.g., docetaxel) thereof: total dose not to exceed 10 mg (range of 0.1 μg to 10 mg); preferred 1 μg to 3 mg. The dose per unit area of the device of 0.1 μg-10 μg per mm²; preferred dose of 0.25 μg/mm²-5 μg/mm². Minimum concentration of 10⁻⁸-10⁻⁴ M of paclitaxel is to be maintained on the device surface. (C) Cell cycle inhibitors such as podophyllotoxins (e.g., etoposide): total dose not to exceed 10 mg (range of 0.1 μg to 10 mg); preferred 1 μg to 3 mg. The dose per unit area of the device of 0.1 μg-10 μg per mm²; preferred dose of 0.25 μg/mm²-5 μg/mm². Minimum concentration of 10⁻⁸-10⁻⁴ M of etoposide is to be maintained on the device surface. (D) Immunomodulators including sirolimus and everolimus. Sirolimus (i.e., rapamycin, RAPAMUNE): Total dose not to exceed 10 mg (range of 0.1 μg to 10 mg); preferred 10 μg to 1 mg. The dose per unit area of 0.1 μg-100 μg per mm²; preferred dose of 0.5 μg/mm²-10 μg/mm². Minimum concentration of 10⁻⁸-10⁻⁴ M is to be maintained on the device surface. Everolimus and derivatives and analogues thereof: Total dose should not exceed 10 mg (range of 0.1 μg to 10 mg); preferred 10 μg to 1 mg. The dose per unit area of 0.1 μg-100 μg per mm² of surface area; preferred dose of 0.3 μg/mm²-10 μg/mm². Minimum concentration of 10⁻⁸-10⁻⁴ M of everolimus is to be maintained on the device surface. (E) Heat shock protein 90 antagonists (e.g., geldanamycin) and analogues and derivatives thereof: total dose not to exceed 20 mg (range of 0.1 μg to 20 mg); preferred 1 μg to 5 mg. The dose per unit area of the device of 0.1 μg-10 μg per mm²; preferred dose of 0.25 μg/mm²-5 μg/mm². Minimum concentration of 10⁻⁸-10⁻⁴ M of geldanamycin is to be maintained on the device surface. (F) HMGCoA reductase inhibitors (e.g., simvastatin) and analogues and derivatives thereof: total dose not to exceed 2000 mg (range of 10.0 μg to 2000 mg); preferred 10 μg to 300 mg. The dose per unit area of the device of 1.0 μg-1000 μg per mm²; preferred dose of 2.5 μg/mm²-500 μg/mm². Minimum concentration of 10⁻⁸-10⁻³ M of simvastatin is to be maintained on the device surface. (G) Inosine monophosphate dehydrogenase inhibitors (e.g., mycophenolic acid, 1-alpha-25 dihydroxy vitamin D₃) and analogues and derivatives thereof: total dose not to exceed 2000 mg (range of 10.0 μg to 2000 mg); preferred 10 μg to 300 mg. The dose per unit area of the device of 1.0 μg-1000 μg per mm²; preferred dose of 2.5 μg/mm²-500 μg/mm². Minimum concentration of 10⁻⁸-10⁻³ M of mycophenolic acid is to be maintained on the device surface. (H)NF kappa B inhibitors (e.g., Bay 11-7082) and analogues and derivatives thereof: total dose not to exceed 200 mg (range of 1.0 μg to 200 mg); preferred 1 μg to 50 mg. The dose per unit area of the device of 1.0 μg-100 μg per mm²; preferred dose of 2.5 μg/mm²-50 μg/mm². Minimum concentration of 10⁻⁸-10⁻⁴ M of Bay 11-7082 is to be maintained on the device surface. (I) Antimycotic agents (e.g., sulconizole) and analogues and derivatives thereof: total dose not to exceed 2000 mg (range of 10.0 μg to 2000 mg); preferred 10 μg to 300 mg. The dose per unit area of the device of 1.0 μg-1000 μg per mm²; preferred dose of 2.5 μg/mm²-500 μg/mm². Minimum concentration of 10⁻⁸-10⁻³ M of sulconizole is to be maintained on the device surface. (J) p38 MAP kinase inhibitors (e.g., SB202190) and analogues and derivatives thereof: total dose not to exceed 2000 mg (range of 10.0 μg to 2000 mg); preferred 10 μg to 300 mg. The dose per unit area of the device of 1.0 μg-1000 μg per mm²; preferred dose of 2.5 μg/mm²-500 μg/mm². Minimum concentration of 10⁻⁸-10⁻³ M of SB202190 is to be maintained on the device surface. (K) Anti-angiogenic agents (e.g., halofuginone bromide) and analogues and derivatives thereof: total dose not to exceed 10 mg (range of 0.1 μg to 10 mg); preferred 1 μg to 3 mg. The dose per unit area of the device of 0.1 μg-10 μg per mm²; preferred dose of 0.25 μg/mm²-5 μg/mm². Minimum concentration of 10⁻⁸-10⁻⁴ M of halofuginone bromide is to be maintained on the device surface.

In addition to those described above (e.g., sirolimus, everolimus, and tacrolimus), several other examples of immunomodulators and appropriate dosages ranges for use with prosthetic heart valve devices include the following: (A) Biolimus and derivatives and analogues thereof: Total dose should not exceed 10 mg (range of 0.1 μg to 10 mg); preferred 10 μg to 1 mg. The dose per unit area of 0.1 μg-100 μg per mm² of surface area; preferred dose of 0.3 μg/mm²-10 μg/mm². Minimum concentration of 10⁻⁸-10⁻⁴ M of everolimus is to be maintained on the device surface. (B) Tresperimus and derivatives and analogues thereof: Total dose should not exceed 10 mg (range of 0.1 μg to 10 mg); preferred 10 μg to 1 mg. The dose per unit area of 0.1 μg-100 μg per mm² of surface area; preferred dose of 0.3 μg/mm²-10 μg/mm². Minimum concentration of 10⁻⁸-10⁻⁴ M of tresperimus is to be maintained on the device surface. (C) Auranofin and derivatives and analogues thereof: Total dose should not exceed 10 mg (range of 0.1 μg to 10 mg); preferred 10 μg to 1 mg. The dose per unit area of 0.1 μg-100 μg per mm² of surface area; preferred dose of 0.3 μg/mm²-10 μg/mm². Minimum concentration of 10⁻⁸-10⁻⁴ M of auranofin is to be maintained on the device surface. (D) 27-0-Demethylrapamycin and derivatives and analogues thereof: Total dose should not exceed 10 mg (range of 0.1 μg to 10 mg); preferred 10 μg to 1 mg. The dose per unit area of 0.1 μg-100 μg per mm² of surface area; preferred dose of 0.3 μg/mm²-10 μg/mm². Minimum concentration of 10⁻⁸-10⁻⁴ M of 27-0-Demethylrapamycin is to be maintained on the device surface. (E) Gusperimus and derivatives and analogues thereof: Total dose should not exceed 10 mg (range of 0.1 μg to 10 mg); preferred 10 μg to 1 mg. The dose per unit area of 0.1 μg-100 μg per mm² of surface area; preferred dose of 0.3 μg/mm²-10 μg/mm². Minimum concentration of 10⁻⁸-10⁻⁴ M of gusperimus is to be maintained on the device surface. (F) Pimecrolimus and derivatives and analogues thereof: Total dose should not exceed 10 mg (range of 0.1 μg to 10 mg); preferred 10 μg to 1 mg. The dose per unit area of 0.1 μg-100 μg per mm² of surface area; preferred dose of 0.3 μg/mm²-10 μg/mm². Minimum concentration of 10⁻⁸-10⁻⁴ M of pimecrolimus is to be maintained on the device surface and (G) ABT-578 and analogues and derivatives thereof: Total dose should not exceed 10 mg (range of 0.1 μg to 10 mg); preferred 10 μg to 1 mg. The dose per unit area of 0.1 μg-100 μg per mm² of surface area; preferred dose of 0.3 μg/mm²-10 μg/mm². Minimum concentration of 10⁻⁸-10⁻⁴ M of ABT-578 is to be maintained on the device surface.

In addition to those described above (e.g., paclitaxel, TAXOTERE, and docetaxel), several other examples of anti-microtubule agents and appropriate dosages ranges for use with prosthetic heart valve devices include vinca alkaloids such as vinblastine and vincristine sulfate and analogues and derivatives thereof: total dose not to exceed 10 mg (range of 0.1 μg to 10 mg); preferred 1 μg to 3 mg. Dose per unit area of the device of 0.1 μg-10 μg per mm²; preferred dose of 0.25 μg/mm²-5 μg/mm². Minimum concentration of 10⁻⁸-10⁻⁴ M of drug is to be maintained on the device surface.

Penile Implants

In one aspect, the present invention provides for the combination of an anti-scarring agent and a penile implant device. In one aspect, penile implants are loaded with an anti-scarring drug or a composition that includes an anti-scarring drug to prevent fibrous encapsulation.

Penile implants are used to treat erectile dysfunction and are generally flexible rods, hinged rods or inflatable devices with a pump. Penile implants may be composed of rods, coils, inflatable tubes and/or pressure chambers and may be used to provide erectile function, enlargement or provide shape to a misshapen or damaged penis. For example, the penile implant may be an implantable polymeric material which is injected into the lamina propria mucosae of the glans in order to enlarge the glans of the male genital organ. See, e.g., U.S. Pat. No. 6,418,934. The penile implant may be composed of a pair of arced, elongated portions made of silicone rubber that are mirror images of each other, which has a varying circumferential wall thickness. See, e.g., U.S. Pat. No. 6,537,204. The penile implant may be used to increase penile volume by being adapted to cover the outer lateral sides of the corpus cavernosum without covering the upper and lower sides thereof. See, e.g., U.S. Pat. No. 6,015,380. The penile implant may be an inflatable, self-contained implant composed of a cylindrical body having a pump that transfers fluid from a reservoir to a pressure chamber that has a pressure relief valve. See, e.g., U.S. Pat. Nos. 4,898,158 and 4,823,779. The penile implant may be composed of an elongated rod having a relatively short proximal stem portion, which is covered by a layer of hydrophilic material that contains a plurality of openings and swells as it absorbs water. See, e.g., U.S. Pat. No. 4,611,584. The penile implant may be composed of at least one inflatable tube that has fluid interchange with a mounting base which is controlled by a manual pump implanted in the scrotum. See, e.g., U.S. Pat. No. 6,475,137. The penile implant may be a flexible double-walled partial cylindrical sleeve that has bellow-like construction which is suited for penile malformation. See, e.g., U.S. Pat. No. 5,669,870. The penile implant may be used for correcting erectile impotence by being composed of at least one flexible portion with a pressure chamber connected by tubing to an accumulator charged with fluid, such that pressurizing fluid flows when the valve is opened. See, e.g., U.S. Pat. No. 4,917,110. The penile implant may be composed of a stainless steel pad supported by a plurality of strands which is surrounded by a cylinder with a silicone ring that can move longitudinally in response to the expansion or shrinkage of the penis. See, e.g., U.S. Pat. No. 5,433,694. The penile implant may increase girth and length by being composed of a cylindrical sleeve that has an elastic outer sheet and an inner inelastic sheet that forms a closed sack to receive a fluid under pressure from a fluid source. See, e.g., U.S. Pat. No. 5,445,594. The penile implant may be composed of a braided sleeve with an outer elastomeric surface and inner surface having grooves and ribs in a helical arrangement, such that the implant is malleable having both a bendable configuration and an unbent rigid configuration. See, e.g., U.S. Pat. No. 5,512,033. The penile implant may be a polymeric matrix having dissociated cartilage-forming cells deposited on and in said matrix whereby a cartilaginous structure is formed upon implantation having controlled biomechanical properties and tensile strength. See, e.g., U.S. Pat. No. 6,547,719. The penile implant may be composed of an implantable supply pump, deformable reservoir, and conducting/dispensing catheters, such that a vasodilator agent is delivered to the erectile bodies to treat male impotence. See, e.g., U.S. Pat. No. 6,679,832. Other penile implants are described in, e.g., U.S. Pat. Nos. 6,579,230; 5,704,895; 5,250,020; 5,048,510 and 4,875,472.

A fibrosis-inhibiting agent may be incorporated into, onto or near the device. Penile implants, which may be combined with one or more agents according to the present invention, include commercially available products, such as, for example, the TITAN Inflatable Penile Prosthesis from Mentor Corporation (Santa Barbara, Calif.) and the AMS penile prosthesis product line including the AMS 700 CX CXM, AMS AMBICOR, and AMS Malleable 600M Penile Prostheses from American Medical Systems, Inc. (Minnetonka, Minn.),

In one aspect, the present invention provides penile implant devices that include an anti-scarring agent or a composition that includes an anti-scarring agent. Numerous polymeric and non-polymeric delivery systems for use in penile implants have been described above. Methods for incorporating the fibrosis-inhibiting agent into or onto the device includes: (a) directly affixing to the device a fibrosis-inhibiting composition (e.g., by either a spraying process or dipping process as described above, with or without a carrier), (b) directly incorporating into the device a fibrosis-inhibiting composition (e.g., by either a spraying process or dipping process as described above, with or without a carrier), (c) by coating the device with a substance such as a hydrogel which will in turn absorb the fibrosis-inhibiting composition, (d) by interweaving fibrosis-inhibiting composition coated thread (or the polymer itself formed into a thread) into the device structure, (e) constructing the device itself or a portion of the device with a fibrosis-inhibiting composition, or (f) by covalently binding the fibrosis-inhibiting agent directly to the device surface or to a linker (small molecule or polymer) that is coated or attached to the device surface. The coatings can be applied to different portions of the device. For example, the coating can be (a) a coating applied to the external surface of the portion of the penile implant that is implanted into the penis; (b) a coating applied to the external surfaces of the portions of the penile implant that are implanted in the scrotum, or (c) a coating applied to all or parts of the surfaces of the entire device.

In addition to coating the device with the fibrosis-inhibiting composition, the fibrosis-inhibiting agent can be mixed with the materials that are used to make the device such that the fibrosis-inhibiting agent is incorporated into the final device.

In addition to incorporation of a fibrosis-inhibiting agent into or onto the device, another biologically active agent can be incorporated into or onto the device, for example an anti-inflammatory (e.g., dexamethazone or aspirin).

In another aspect, the device may further comprise an antibiotic or a combination of an antibiotic and an anti-inflammatory agent in order to combat infection associated with implantation of penile implants.

The placement of penile implants can be complicated by infection (usually in the first 6 months after surgery) with Coagulase Negative Staphylococci (including Staphylococcus epidermidis), Staphylococcus aureus, Pseudomonas aeruginosa, Enterococci, Serratia and Candida. Infection is characterized by fever, erythema, induration and purulent drainage from the operative site. The usual route of infection is through the incision at the time of surgery and up to 3% of penile implants become infected despite the best sterile surgical technique. To help combat this, intraoperative irrigation with antibiotic solutions is often employed.

Drug-coating of, or drug incorporation into, the penile implant can allow bacteriocidal drug levels to be achieved locally, thus reducing the incidence of bacterial colonization (and subsequent development of local infection and device failure), while producing negligible systemic exposure to the drugs.

Representative examples of antibiotics include amoxicillin, trimethoprim-sulfamethoxazole, azithromycin, clarithromycin, amoxicillin-clavulanate, cefprozil, cefuroxime, cefpodoxime, or cefdinir).

Other examples of anti-infective compounds include doxorubicin, mitoxantrone, 5-fluorouracil and etoposide.

Utilizing the fluoropyrimidine, 5-fluorouracil, as an example, whether applied as a polymer coating, incorporated into the polymers which make up the implant, or applied without a carrier polymer, the total dose of 5-fluorouracil applied should not exceed 250 mg (range of 1.0 μg to 250 mg). In a particularly preferred embodiment, the total amount of drug applied should be in the range of 10 μg to 25 mg. The dose per unit area (i.e., the amount of drug as a function of the surface area of the portion of the implant to which drug is applied and/or incorporated) should fall within the range of 0.1 μg-1 mg per mm² of surface area. In a particularly preferred embodiment, 5-fluorouracil should be applied to the implant surface at a dose of 1.0 μg/mm²-50 μg/mm². As different polymer and non-polymer coatings will release 5-fluorouracil at differing rates, the above dosing parameters should be utilized in combination with the release rate of the drug from the implant surface such that a minimum concentration of 10⁻⁴-10⁻⁷ M of 5-fluorouracil is maintained. It is necessary to insure that surface drug concentrations exceed concentrations of 5-fluorouracil known to be lethal to numerous species of bacteria and fungi (i.e., are in excess of 10⁻⁴ M; although for some embodiments lower drug levels will be sufficient). In a preferred embodiment, 5-fluorouracil is released from the implant surface such that anti-infective activity is maintained for a period ranging from several hours to several months. In a particularly preferred embodiment the drug is released in effective concentrations for a period ranging from 1 week-6 months. It should be readily evident based upon the discussions provided herein that analogues and derivatives of 5-fluorouracil (as described previously) with similar functional activity can be utilized for the purposes of this invention; the above dosing parameters are then adjusted according to the relative potency of the analogue or derivative as compared to the parent compound (e.g., a compound twice as potent as 5-fluorouracil is administered at half the above parameters, a compound half as potent as 5-fluorouracil is administered at twice the above parameters, etc.).

Anti-inflammatory and anti-infective agents may be formulated, for example, into a coating applied to the surface of the penile implant. The drug(s) can be applied in several manners: (a) as a coating applied to the external surface of the penile implant; and/or (b) incorporated into the polymers which comprise the penile implant.

According to the present invention, any fibrosis-inhibiting agent described above can be utilized in the practice of this embodiment. Within one embodiment of the invention, penile implants may be adapted to release an agent that inhibits one or more of the four general components of the process of fibrosis (or scarring), including: formation of new blood vessels (angiogenesis), migration and proliferation of connective tissue cells (such as fibroblasts or smooth muscle cells), deposition of extracellular matrix (ECM), and remodeling (maturation and organization of the fibrous tissue). By inhibiting one or more of the components of fibrosis (or scarring), the overgrowth of granulation tissue may be inhibited or reduced.

As penile implant devices are made in a variety of configurations and sizes, the exact dose administered will vary with device size, surface area and design. However, certain principles can be applied in the application of this art. Drug dose can be calculated as a function of dose per unit area (of the portion of the device being coated), total dose administered, and appropriate surface concentrations of active drug can be determined. Drugs are to be used at concentrations that range from several times more than to 10%, 5%, or even less than 1% of the concentration typically used in a single chemotherapeutic systemic dose application. Preferably, the drug is released in effective concentrations for a period ranging from 1-90 days.

Several examples of fibrosis-inhibiting agents for use in penile implants include the following: cell cycle inhibitors including (A) anthracyclines (e.g., doxorubicin and mitoxantrone), (B) taxanes (e.g., paclitaxel, TAXOTERE and docetaxel), and (C) podophyllotoxins (e.g., etoposide); (D) immunomodulators (e.g., sirolimus, everolimus, tacrolimus); (E) heat shock protein 90 antagonists (e.g., geldanamycin); (F) HMGCoA reductase inhibitors (e.g., simvastatin); (G) inosine monophosphate dehydrogenase inhibitors (e.g., mycophenolic acid, 1-alpha-25 dihydroxy vitamin D₃); (H)NF kappa B inhibitors (e.g., Bay 11-7082); (I) antimycotic agents (e.g., sulconizole), (J) p38 MAP kinase inhibitors (e.g., SB202190), and (K) and anti-angiogenesis agents (e.g., halofuginone bromide), as well as analogues and derivatives of the aforementioned.

Regardless of the method of application of the drug to the penile implant, the exemplary anti-fibrosing agents, used alone or in combination, should be administered under the following dosing guidelines. The total amount (dose) of anti-scarring agent in or on the penile implant may be in the range of about 0.01 μg-10 μg, or 10 μg-10 mg, or 10 mg-250 mg, or 250 mg-1000 mg, or 1000 mg-2500 mg. The dose (amount) of anti-scarring agent per unit area of device surface to which the agent is applied may be in the range of about 0.01 μg/mm²-1 μg/mm², or 1 μg/mm²-10 μg/mm², or 10 μg/mm²-250 μg/mm², 250 μg/mm²-1000 μg/mm², or 1000 μg/mm²-2500 μg/mm².

Provided below are exemplary dosage ranges for various anti-scarring agents that can be used in conjunction with penile implant devices in accordance with the invention. A) Cell cycle inhibitors including doxorubicin and mitoxantrone. Doxorubicin analogues and derivatives thereof: total dose not to exceed 25 mg (range of 0.1 μg to 25 mg); preferred 1 μg to 5 mg. The dose per unit area of 0.01 μg-100 μg per mm²; preferred dose of 0.1 μg/mm²-10 μg/mm². Minimum concentration of 10⁻⁸-10⁻⁴ M of doxorubicin is to be maintained on the device surface. Mitoxantrone and analogues and derivatives thereof: total dose not to exceed 5 mg (range of 0.01 μg to 5 mg); preferred 0.1 μg to 1 mg. The dose per unit area of the device of 0.01 μg-20 μg per mm²; preferred dose of 0.05 μg/mm²-3 μg/mm². Minimum concentration of 10⁻⁸-10⁻⁴ M of mitoxantrone is to be maintained on the device surface. B) Cell cycle inhibitors including Paclitaxel and analogues and derivatives (e.g., docetaxel) thereof: total dose not to exceed 10 mg (range of 0.1 μg to 10 mg); preferred 1 μg to 3 mg. The dose per unit area of the device of 0.1 μg-10 μg per mm²; preferred dose of 0.25 μg/mm²-5 μg/mm². Minimum concentration of 10⁻⁸-10⁻⁴ M of paclitaxel is to be maintained on the device surface. (C) Cell cycle inhibitors such as podophyllotoxins (e.g., etoposide): total dose not to exceed 10 mg (range of 0.1 μg to 10 mg); preferred 1 μg to 3 mg. The dose per unit area of the device of 0.1 μg-10 μg per mm²; preferred dose of 0.25 μg/mm²-5 μg/mm². Minimum concentration of 10⁻⁸-10⁴ M of etoposide is to be maintained on the device surface. (D) Immunomodulators including sirolimus and everolimus. Sirolimus (i.e., rapamycin, RAPAMUNE): Total dose not to exceed 10 mg (range of 0.1 μg to 10 mg); preferred 10 μg to 1 mg. The dose per unit area of 0.1 μg-100 μg per mm²; preferred dose of 0.5 μg/mm²-10 μg/mm². Minimum concentration of 10⁻⁸-10⁻⁴ M is to be maintained on the device surface. Everolimus and derivatives and analogues thereof: Total dose should not exceed 10 mg (range of 0.1 μg to 10 mg); preferred 10 μg to 1 mg. The dose per unit area of 0.1 μg-100 μg per mm² of surface area; preferred dose of 0.3 μg/mm²-10 μg/mm². Minimum concentration of 10⁻⁸-10⁻⁴ M of everolimus is to be maintained on the device surface. (E) Heat shock protein 90 antagonists (e.g., geldanamycin) and analogues and derivatives thereof: total dose not to exceed 20 mg (range of 0.1 μg to 20 mg); preferred 1 μg to 5 mg. The dose per unit area of the device of 0.1 μg-10 μg per mm²; preferred dose of 0.25 μg/mm²-5 μg/mm². Minimum concentration of 10⁸-10⁻⁴ M of geldanamycin is to be maintained on the device surface. (F) HMGCoA reductase inhibitors (e.g., simvastatin) and analogues and derivatives thereof: total dose not to exceed 2000 mg (range of 10.0 μg to 2000 mg); preferred 10 μg to 300 mg. The dose per unit area of the device of 1.0 μg-1000 μg per mm²; preferred dose of 2.5 μg/mm²-500 μg/mm². Minimum concentration of 10⁻⁸-10⁻³ M of simvastatin is to be maintained on the device surface. (G) Inosine monophosphate dehydrogenase inhibitors (e.g., mycophenolic acid, 1-alpha-25 dihydroxy vitamin D₃) and analogues and derivatives thereof: total dose not to exceed 2000 mg (range of 10.0 μg to 2000 mg); preferred 10 μg to 300 mg. The dose per unit area of the device of 1.0 μg-1000 μg per mm²; preferred dose of 2.5 μg/mm²-500 μg/mm². Minimum concentration of 10⁻⁸-10⁻³ M of mycophenolic acid is to be maintained on the device surface. (H)NF kappa B inhibitors (e.g., Bay 11-7082) and analogues and derivatives thereof: total dose not to exceed 200 mg (range of 1.0 μg to 200 mg); preferred 1 μg to 50 mg. The dose per unit area of the device of 1.0 μg-100 μg per mm²; preferred dose of 2.5 μg/mm²-50 μg/mm². Minimum concentration of 10⁻⁸-10⁻⁴ M of Bay 11-7082 is to be maintained on the device surface. (I) Antimycotic agents (e.g., sulconizole) and analogues and derivatives thereof: total dose not to exceed 2000 mg (range of 10.0 μg to 2000 mg); preferred 10 μg to 300 mg. The dose per unit area of the device of 1.0 μg-1000 μg per mm²; preferred dose of 2.5 μg/mm²-500 μg/mm². Minimum concentration of 10⁻⁸-10⁻³ M of sulconizole is to be maintained on the device surface. (J) p38 MAP kinase inhibitors (e.g., SB202190) and analogues and derivatives thereof: total dose not to exceed 2000 mg (range of 10.0 μg to 2000 mg); preferred 10 μg to 300 mg. The dose per unit area of the device of 1.0 μg-1000 μg per mm²; preferred dose of 2.5 μg/mm²-500 μg/mm². Minimum concentration of 10⁻⁸-10⁻³ M of SB202190 is to be maintained on the device surface. (K) Anti-angiogenic agents (e.g., halofuginone bromide) and analogues and derivatives thereof: total dose not to exceed 10 mg (range of 0.1 μg to 10 mg); preferred 1 μg to 3 mg. The dose per unit area of the device of 0.01 μg-10 μg per mm²; preferred dose of 0.25 μg/mm²-5 μg/mm². Minimum concentration of 10⁻⁸-10⁻⁴ M of halofuginone bromide is to be maintained on the device surface.

In addition to those described above (e.g., sirolimus, everolimus, and tacrolimus), several other examples of immunomodulators and appropriate dosages ranges for use with penile implant devices include the following: (A) Biolimus and derivatives and analogues thereof: Total dose should not exceed 10 mg (range of 0.1 μg to 10 mg); preferred 10 μg to 1 mg. The dose per unit area of 0.1 μg-100 μg per mm² of surface area; preferred dose of 0.3 μg/mm²-10 μg/mm². Minimum concentration of 10⁻⁸-10⁻⁴ M of everolimus is to be maintained on the device surface. (B) Tresperimus and derivatives and analogues thereof: Total dose should not exceed 10 mg (range of 0.1 μg to 10 mg); preferred 10 μg to 1 mg. The dose per unit area of 0.1 μg-100 μg per mm² of surface area; preferred dose of 0.3 μg/mm²-10 μg/mm². Minimum concentration of 10⁻⁸-10⁻⁴ M of tresperimus is to be maintained on the device surface. (C) Auranofin and derivatives and analogues thereof: Total dose should not exceed 10 mg (range of 0.1 μg to 10 mg); preferred 10 μg to 1 mg. The dose per unit area of 0.1 μg-100 μg per mm² of surface area; preferred dose of 0.3 μg/mm²-10 μg/mm². Minimum concentration of 10⁻⁸-10⁻⁴ M of auranofin is to be maintained on the device surface. (D) 27-0-Demethylrapamycin and derivatives and analogues thereof: Total dose should not exceed 10 mg (range of 0.1 μg to 10 mg); preferred 10 μg to 1 mg. The dose per unit area of 0.1 μg-100 μg per mm² of surface area; preferred dose of 0.3 μg/mm²-10 μg/mm². Minimum concentration of 10⁻⁸-10⁻⁴ M of 27-0-Demethylrapamycin is to be maintained on the device surface. (E) Gusperimus and derivatives and analogues thereof: Total dose should not exceed 10 mg (range of 0.1 μg to 10 mg); preferred 10 μg to 1 mg. The dose per unit area of 0.1 μg-100 μg per mm² of surface area; preferred dose of 0.3 μg/mm²-10 μg/mm². Minimum concentration of 10⁻⁸-10⁻⁴ M of gusperimus is to be maintained on the device surface. (F) Pimecrolimus and derivatives and analogues thereof: Total dose should not exceed 10 mg (range of 0.1 μg to 10 mg); preferred 10 μg to 1 mg. The dose per unit area of 0.1 μg-100 μg per mm² of surface area; preferred dose of 0.3 μg/mm²-10 μg/mm². Minimum concentration of 10⁻⁸-10⁻⁴ M of pimecrolimus is to be maintained on the device surface and (G) ABT-578 and analogues and derivatives thereof: Total dose should not exceed 10 mg (range of 0.1 μg to 10 mg); preferred 10 μg to 1 mg. The dose per unit area of 0.1 μg-100 μg per mm² of surface area; preferred dose of 0.3 μg/mm²-10 μg/mm². Minimum concentration of 10⁻⁸-10⁻⁴ M of ABT-578 is to be maintained on the device surface.

In addition to those described above (e.g., paclitaxel, TAXOTERE, and docetaxel), several other examples of anti-microtubule agents and appropriate dosages ranges for use with penile implant devices include vinca alkaloids such as vinblastine and vincristine sulfate and analogues and derivatives thereof: total dose not to exceed 10 mg (range of 0.1 μg to 10 mg); preferred 1 μg to 3 mg. Dose per unit area of the device of 0.1 μg-10 μg per mm²; preferred dose of 0.25 μg/mm²-5 μg/mm². Minimum concentration of 10⁻⁸-10⁻⁴ M of drug is to be maintained on the device surface.

Endotracheal and Tracheostomy Tubes

In one aspect, the present invention provides for the combination of an anti-scarring agent and endotracheal and tracheostomy tube devices. Association of an anti-scarring agent with an endotracheal or a tracheostomy tube (e.g., chest tube) may be used to prevent stenosis of the artificial airway.

Endotracheal tubes and tracheostomy tubes are used to maintain the airway when ventilatory assistance is required. Endotracheal tubes tend to be used to establish an airway in the acute setting, while tracheostomy tubes are used when prolonged ventilation is required or when there is a fixed obstruction in the upper airway.

In one aspect, endotracheal tubes may be used to provide a mechanical air passageway, which may be required for ventilation of the lungs during injury or surgery. Endotracheal tubes may have a single lumen or double lumen, and may have a flange or balloon for engaging its position within the trachea. For example, the endotracheal tube may be composed of an inner and outer flexible tube having a radially extending flange that prevents advancement beyond the larynx. See, e.g., U.S. Pat. No. 5,259,371. The endotracheal tube may have a double lumen which is removably affixed whereby the first tubular lumen may be removed from the airway while the second tubular lumen remains intact. See, e.g., U.S. Pat. No. 6,443,156. The endotracheal tube may have a tracheal portion and a bronchial portion attached at an angle that forms a single lumen, whereby when a balloon that is positioned within the tube is inflated, it blocks the flow of gas through the bronchial portion. See, e.g., U.S. Pat. No. 6,609,521. The endotracheal tube may be composed of two cylindrical portions of different diameters which are connected by a non-circularly shaped tapered portion to complement the glottis which has a plurality of sealing gills that are thin and pliable that extends from the tapered portion. See, e.g., U.S. Pat. No. 5,429,127. The endotracheal tube may be composed of a tubular portion with a visual indicator to provide guidance of the rotational orientation of the beveled tip at the distal end as it is advanced along the airway. See, e.g., U.S. Pat. No. 6,568,393. The endotracheal tube may be composed of a light reflective coated bore to enhance image transmission and a flexible plurality of passages, one adapted to receive a fiber optic bundle, another connected to an inflatable cuff, and another adapted to receive a malleable stylette to aid in insertion and removal. See, e.g., U.S. Pat. No. 6,629,924. The endotracheal tube may be composed of a hollow, flexible, cylindrical tube having an annular flange at its tip and a connector with an annular internal ridge that is concentrically mounted upon the outer proximal surface of the tube portion. See, e.g., U.S. Pat. No. 5,251,617. The endotracheal tube may be composed of a main tube with an inflatable cuff for sealing, which has a double lumen for irrigation and suction for removal of secretions that may pool in the trachea. See, e.g., U.S. Pat. No. 5,143,062. Other endotracheal tubes are described in, e.g., U.S. Pat. Nos. 6,321,749; 5,765,559; 5,353,787; 5,291,882 and 4,977,894.

Tracheostomy tubes can be used to provide a bypass supply of air when the throat is obstructed. Tracheostomy tubes are used with an obturator for percutaneous insertion into a trachea through a stoma in the neck between adjacent cartilages to assist breathing. For example, the tracheostomy tube may be a tubular cannula formed of soft flexible plastic material which has a tapered distal end that is beveled, narrow, angled and curved downwardly for positioning within the trachea. See, e.g., U.S. Pat. No. 5,058,580. The tracheostomy tube may be composed of a tube with a removable fitting mounted on the exposed end which may be sealed to the tube. See, e.g., U.S. Pat. No. 5,606,966. The tracheostomy tube may be composed of an arcuate cannula with a flange that extends laterally outward and a rotatable tubular elbow that has a fluid connection with the cannula. See, e.g., U.S. Pat. Nos. 5,259,376 and 5,054,482. The tracheostomy tube may be composed of two airways with a pneumatic vibrator that generates sonic vibrations to permit audible speech. See, e.g., U.S. Pat. No. 4,773,412. The tracheostomy tube may be composed of an inner cannula removably received within an outer cannula with a sealing cuff between the outer cannula and the trachea to substantially prevent air from escaping from the trachea and to allow phonation through a secondary passageway formed between the inner and outer cannula. See, e.g., U.S. Pat. No. 4,573,460. The tracheostomy tube may be composed of a first port for orienting outside the neck of the wearer, a second port for orienting within the trachea, and a third connecting port to provide and control gas flow via a valve. See, e.g., U.S. Pat. No. 5,957,978. The tracheostomy tube may be composed of a hollow tube, an inflatable balloon having orthogonal projections, and a flange that provides an anchor external to the throat. See, e.g., U.S. Pat. No. 6,612,305. The tracheostomy tube may be composed of a highly flexible material having wire reinforcement and a neck plate with a collar portion that may slide along the tube. See, e.g., U.S. Pat. No. 5,443,064. Other tracheostomy tubes are described in, e.g., U.S. Pat. Nos. 6,662,804; 6,135,110 and 5,983,895.

Endotracheal tubes, which may be combined with one or more agents according to the present invention, include commercially available products, such as the HI-LO Tracheal Tubes, LASER-FLEX Tracheal Tubes, and ENDOTROL Tracheal Tubes from Nellcor Puritan Bennett Inc. (Pleasanton, Calif.), the SHERIDAN Endotracheal Tubes from Hudson RCI (Temecula, Calif.), and the BARD Endotracheal Tube, Cuffed from C.R. Bard, Inc. (Murray Hill, N.J.).

Tracheostomy tubes, which may be combined with one or more agents according to the present invention, include commercially available products, such as the SHILEY TRACHEOSOFT XLT Tracheostomy Tubes, PHONATE Speaking Valves, and Reusable Cannula Cuffless Tracheostomy Tubes from Nellcor Puritan Bennett Inc. (Pleasanton, Calif.), the PER-FIT Percutaneous Dilational Tracheostomy Kits, PORTEX BLUE LINE Cuffed Tracheostomy Tubes, and BIVONA Uncuffed Tracheostomy Tubes from Portex, Inc. (Keene, N.H.), and the CRYSTALCLEAR Tracheostomy Tubes from Rusch (Germany).

In one aspect, the present invention provides endotracheal and tracheostomy tube devices that include an anti-scarring agent or a composition that includes an anti-scarring agent. Numerous polymeric and non-polymeric delivery systems for use in endotracheal and tracheostomy devices have been described above. Methods for incorporating the fibrosis-inhibiting agent into or onto the device includes: (a) directly affixing to the device a fibrosis-inhibiting composition (e.g., by either a spraying process or dipping process as described above, with or without a carrier), (b) directly incorporating into the device a fibrosis-inhibiting composition (e.g., by either a spraying process or dipping process as described above, with or without a carrier), (c) by coating the device with a substance such as a hydrogel which will in turn absorb the fibrosis-inhibiting composition, (d) by interweaving fibrosis-inhibiting composition coated thread (or the polymer itself formed into a thread) into the device structure, (e) constructing the device itself or a portion of the device with a fibrosis-inhibiting composition, or (f) by covalently binding the fibrosis-inhibiting agent directly to the device surface or to a linker (small molecule or polymer) that is coated or attached to the device surface. The coatings can be applied to different portions of the device. For example, the coating can be (a) as a coating applied to the internal (luminal) surface of the endotracheal tube or tracheostomy tube; (b) as a coating applied to the external surface of the endotracheal tube or tracheostomy tube; or (c) as a coating applied to all or parts of both surfaces.

The fibrosis-inhibiting agent can be mixed with the materials that are used to make the device such that the fibrosis-inhibiting agent is incorporated into the final device.

In addition to incorporation of a fibrosis-inhibiting agent into or onto the device, another biologically active agent can be incorporated into or onto the device, for example an anti-inflammatory (e.g., dexamethazone or aspirin) and/or an antibiotic (e.g., amoxicillin, trimethoprim-sulfamethoxazole, azithromycin, clarithromycin, amoxicillin-clavulanate, cefprozil, cefuroxime, cefpodoxime, or cefdinir).

According to the present invention, any fibrosis-inhibiting agent described above can be utilized in the practice of this embodiment. Within one embodiment of the invention, endotracheal and tracheostomy devices may be adapted to release an agent that inhibits one or more of the four general components of the process of fibrosis (or scarring), including: formation of new blood vessels (angiogenesis), migration and proliferation of connective tissue cells (such as fibroblasts or smooth muscle cells), deposition of extracellular matrix (ECM), and remodeling (maturation and organization of the fibrous tissue). By inhibiting one or more of the components of fibrosis (or scarring), the overgrowth of granulation tissue may be inhibited or reduced.

As endotracheal and tracheostomy tube devices are made in a variety of configurations and sizes, the exact dose administered will vary with device size, surface area and design. However, certain principles can be applied in the application of this art. Drug dose can be calculated as a function of dose per unit area (of the portion of the device being coated), total dose administered, and appropriate surface concentrations of active drug can be determined. Drugs are to be used at concentrations that range from several times more than to 10%, 5%, or even less than 1% of the concentration typically used in a single chemotherapeutic systemic dose application. Preferably, the drug is released in effective concentrations for a period ranging from 1-90 days.

Several examples of fibrosis-inhibiting agents for use in endotracheal and tracheostomy tube devices include the following: cell cycle inhibitors including (A) anthracyclines (e.g., doxorubicin and mitoxantrone), (B) taxanes (e.g., paclitaxel, TAXOTERE and docetaxel), and (C) podophyllotoxins (e.g., etoposide); (D) immunomodulators (e.g., sirolimus, everolimus, tacrolimus); (E) heat shock protein 90 antagonists (e.g., geldanamycin); (F) HMGCoA reductase inhibitors (e.g., simvastatin); (G) inosine monophosphate dehydrogenase inhibitors (e.g., mycophenolic acid, 1-alpha-25 dihydroxy vitamin D₃); (H)NF kappa B inhibitors (e.g., Bay 11-7082); (I) antimycotic agents (e.g., sulconizole), (J) p38 MAP kinase inhibitors (e.g., SB202190), and (K) and anti-angiogenesis agents (e.g., halofuginone bromide), as well as analogues and derivatives of the aforementioned.

Regardless of the method of application of the drug to the device, the exemplary anti-fibrosing agents, used alone or in combination, should be administered under the following dosing guidelines. The total amount (dose) of anti-scarring agent in or on the device may be in the range of about 0.01 μg-10 μg, or 10 μg-10 mg, or 10 mg-250 mg, or 250 mg-1000 mg, or 1000 mg-2500 mg. The dose (amount) of anti-scarring agent per unit area of device surface to which the agent is applied may be in the range of about 0.01 μg/mm²-1 μg/mm², or 1 μg/mm²-10 μg/mm², or 10 μg/mm²-250 μg/mm², 250 μg/mm²-1000 μg/mm², or 1000 μg/mm²-2500 μg/mm².

Provided below are exemplary dosage ranges for various anti-scarring agents that can be used in conjunction with endotracheal and tracheostomy tube devices in accordance with the invention. A) Cell cycle inhibitors including doxorubicin and mitoxantrone. Doxorubicin analogues and derivatives thereof: total dose not to exceed 25 mg (range of 0.1 μg to 25 mg); preferred 1 μg to 5 mg. The dose per unit area of 0.01 μg-100 μg per mm²; preferred dose of 0.1 μg/mm²-10 μg/mm². Minimum concentration of 10⁻⁸-10⁻⁴ M of doxorubicin is to be maintained on the device surface. Mitoxantrone and analogues and derivatives thereof: total dose not to exceed 5 mg (range of 0.01 μg to 5 mg); preferred 0.1 μg to 1 mg. The dose per unit area of the device of 0.01 μg-20 μg per mm²; preferred dose of 0.05 μg/mm²-3 μg/mm². Minimum concentration of 10⁻⁸-10⁻⁴ M of mitoxantrone is to be maintained on the device surface. B) Cell cycle inhibitors including paclitaxel and analogues and derivatives (e.g., docetaxel) thereof: total dose not to exceed 10 mg (range of 0.1 μg to 10 mg); preferred 1 μg to 3 mg. The dose per unit area of the device of 0.1 μg-10 μg per mm²; preferred dose of 0.25 μg/mm²-5 μg/mm². Minimum concentration of 10⁻⁸-10⁻⁴ M of paclitaxel is to be maintained on the device surface. (C) Cell cycle inhibitors such as podophyllotoxins (e.g., etoposide): total dose not to exceed 10 mg (range of 0.1 μg to 10 mg); preferred 1 μg to 3 mg. The dose per unit area of the device of 0.1 μg-10 μg per mm²; preferred dose of 0.25 μg/mm²-5 μg/mm². Minimum concentration of 10⁻⁸-10⁻⁴ M of etoposide is to be maintained on the device surface. (D) Immunomodulators including sirolimus and everolimus. Sirolimus (i.e., rapamycin, RAPAMUNE): Total dose not to exceed 10 mg (range of 0.1 μg to 10 mg); preferred 10 μg to 1 mg. The dose per unit area of 0.1 μg-100 μg per mm²; preferred dose of 0.5 μg/mm²-10 μg/mm². Minimum concentration of 10⁻⁸-10⁻⁴ M is to be maintained on the device surface. Everolimus and derivatives and analogues thereof: Total dose should not exceed 10 mg (range of 0.1 μg to 10 mg); preferred 10 μg to 1 mg. The dose per unit area of 0.1 μg-100 μg per mm² of surface area; preferred dose of 0.3 μg/mm²-10 μg/mm². Minimum concentration of 10⁻⁸-10⁻⁴ M of everolimus is to be maintained on the device surface. (E) Heat shock protein 90 antagonists (e.g., geldanamycin) and analogues and derivatives thereof: total dose not to exceed 20 mg (range of 0.1 μg to 20 mg); preferred 1 μg to 5 mg. The dose per unit area of the device of 0.1 μg-10 μg per mm²; preferred dose of 0.25 μg/mm²-5 μg/mm². Minimum concentration of 10⁻⁸-10⁴ M of geldanamycin is to be maintained on the device surface. (F) HMGCoA reductase inhibitors (e.g., simvastatin) and analogues and derivatives thereof: total dose not to exceed 2000 mg (range of 10.0 μg to 2000 mg); preferred 10 μg to 300 mg. The dose per unit area of the device of 1.0 μg-1000 μg per mm²; preferred dose of 2.5 μg/mm²-500 μg/mm². Minimum concentration of 10⁻⁸-10⁻³ M of simvastatin is to be maintained on the device surface. (G) Inosine monophosphate dehydrogenase inhibitors (e.g., mycophenolic acid, 1-alpha-25 dihydroxy vitamin D₃) and analogues and derivatives thereof: total dose not to exceed 2000 mg (range of 10.0 μg to 2000 mg); preferred 10 μg to 300 mg. The dose per unit area of the device of 1.0 μg-1000 μg per mm²; preferred dose of 2.5 μg/mm²-500 μg/mm². Minimum concentration of 10⁻⁸-10⁻³ M of mycophenolic acid is to be maintained on the device surface. (H)NF kappa B inhibitors (e.g., Bay 11-7082) and analogues and derivatives thereof: total dose not to exceed 200 mg (range of 1.0 μg to 200 mg); preferred 1 μg to 50 mg. The dose per unit area of the device of 1.0 μg-100 μg per mm²; preferred dose of 2.5 μg/mm²-50 μg/mm². Minimum concentration of 10⁻⁸-10⁻⁴ M of Bay 11-7082 is to be maintained on the device surface. (I) Antimycotic agents (e.g., sulconizole) and analogues and derivatives thereof: total dose not to exceed 2000 mg (range of 10.0 μg to 2000 mg); preferred 10 μg to 300 mg. The dose per unit area of the device of 1.0 μg-1000 μg per mm²; preferred dose of 2.5 μg/mm²-500 μg/mm². Minimum concentration of 10⁻⁸-10⁻³ M of sulconizole is to be maintained on the device surface. (J) p38 MAP kinase inhibitors (e.g., SB202190) and analogues and derivatives thereof: total dose not to exceed 2000 mg (range of 10.0 μg to 2000 mg); preferred 10 μg to 300 mg. The dose per unit area of the device of 1.0 μg-1000 μg per mm²; preferred dose of 2.5 μg/mm²-500 μg/mm². Minimum concentration of 10⁻⁸-10⁻³ M of SB202190 is to be maintained on the device surface. (K) Anti-angiogenic agents (e.g., halofuginone bromide) and analogues and derivatives thereof: total dose not to exceed 10 mg (range of 0.1 μg to 10 mg); preferred 1 μg to 3 mg. The dose per unit area of the device of 0.1 μg-10 μg per mm²; preferred dose of 0.25 μg/mm²-5 μg/mm². Minimum concentration of 10⁻⁸-10⁻⁴ M of halofuginone bromide is to be maintained on the device surface.

In addition to those described above (e.g., sirolimus, everolimus, and tacrolimus), several other examples of immunomodulators and appropriate dosages ranges for use with endotracheal and tracheostomy devices include the following: (A) Biolimus and derivatives and analogues thereof: Total dose should not exceed 10 mg (range of 0.1 μg to 10 mg); preferred 10 μg to 1 mg. The dose per unit area of 0.1 μg-100 μg per mm² of surface area; preferred dose of 0.3 μg/mm²-10 μg/mm². Minimum concentration of 10⁻⁸-10⁻⁴ M of everolimus is to be maintained on the device surface. (B) Tresperimus and derivatives and analogues thereof: Total dose should not exceed 10 mg (range of 0.1 μg to 10 mg); preferred 10 μg to 1 mg. The dose per unit area of 0.1 μg-100 μg per mm² of surface area; preferred dose of 0.3 μg/mm²-10 μg/mm². Minimum concentration of 10⁻⁸-10⁻⁴ M of tresperimus is to be maintained on the device surface. (C) Auranofin and derivatives and analogues thereof: Total dose should not exceed 10 mg (range of 0.1 μg to 10 mg); preferred 10 μg to 1 mg. The dose per unit area of 0.1 μg-100 μg per mm² of surface area; preferred dose of 0.3 μg/mm²-10 μg/mm². Minimum concentration of 10⁻⁸-10⁻⁴ M of auranofin is to be maintained on the device surface. (D) 27-0-Demethylrapamycin and derivatives and analogues thereof: Total dose should not exceed 10 mg (range of 0.1 μg to 10 mg); preferred 10 μg to 1 mg. The dose per unit area of 0.1 μg-100 μg per mm² of surface area; preferred dose of 0.3 μg/mm²-10 μg/mm². Minimum concentration of 10⁻⁸-10⁻⁴ M of 27-0-Demethylrapamycin is to be maintained on the device surface. (E) Gusperimus and derivatives and analogues thereof: Total dose should not exceed 10 mg (range of 0.1 μg to 10 mg); preferred 10 μg to 1 mg. The dose per unit area of 0.1 μg-100 μg per mm² of surface area; preferred dose of 0.3 μg/mm²-10 μg/mm². Minimum concentration of 10⁻⁸-10⁻⁴ M of gusperimus is to be maintained on the device surface. (F) Pimecrolimus and derivatives and analogues thereof: Total dose should not exceed 10 mg (range of 0.1 μg to 10 mg); preferred 10 μg to 1 mg. The dose per unit area of 0.1 μg-100 μg per mm² of surface area; preferred dose of 0.3 μg/mm²-10 μg/mm². Minimum concentration of 10⁻⁸-10⁻⁴ M of pimecrolimus is to be maintained on the device surface and (G) ABT-578 and analogues and derivatives thereof: Total dose should not exceed 10 mg (range of 0.1 μg to 10 mg); preferred 10 μg to 1 mg. The dose per unit area of 0.1 μg-100 μg per mm² of surface area; preferred dose of 0.3 μg/mm²-10 μg/mm². Minimum concentration of 10⁻⁸-10⁻⁴ M of ABT-578 is to be maintained on the device surface.

In addition to those described above (e.g., paclitaxel, TAXOTERE, and docetaxel), several other examples of anti-microtubule agents and appropriate dosages ranges for use with endotracheal and tracheostomy tube devices include vinca alkaloids such as vinblastine and vincristine sulfate and analogues and derivatives thereof: total dose not to exceed 10 mg (range of 0.1 μg to 10 mg); preferred 1 μg to 3 mg. Dose per unit area of the device of 0.1 μg-10 μg per mm²; preferred dose of 0.25 μg/mm²-5 μg/mm². Minimum concentration of 10⁻⁸-10⁻⁴ M of drug is to be maintained on the device surface.

Peritoneal Dialysis Catheters

In one aspect, the present invention provides for the combination of an anti-scarring agent and a peritoneal dialysis catheter or a peritoneal implant for drug delivery.

Peritoneal catheters may be used for peritoneal dialysis. Peritoneal dialysis is a form of dialysis in which the blood is not removed from the body but instead, cleansing fluid is put into the abdominal cavity where the body's peritoneum acts as the dialysis membrane. The dialysate equilibrates with plasma for several hours and then the equilibrated dialysate is drained with the associated toxins. The peritoneal catheter is surgically placed into the peritoneal cavity in order to drain dialysate into and out of the peritoneal cavity.

Peritoneal dialysis catheters are typically double-cuffed and tunnelled catheters that provide access to the peritoneum. The most common peritoneal dialysis catheter designs are the Tenckhoff catheter, the Swan Neck Missouri catheter and the Toronto Western catheter. In peritoneal dialysis, the peritoneum acts as a semipermeable membrane across which solutes can be exchanged down a concentration gradient. Continuous peritoneal access catheters are permanently implanted for those that require repeated access to the peritoneum. Implanted peritoneal catheters may be used for peritoneal dialysis or for a means of delivering drug to the peritoneum. These catheters may be composed of synthetic materials, such as silicone, rubber, polyurethane or other polymers that provide flexibility. They may be designed to be configured as a straight tube or may be bent and molded into a variety of shapes to provide different configurations, including helices and coils. The peritoneal catheters may be composed of one continuous element or may be sectioned into parts to provide flanges, cuffs, beads or discs at one of the ends to fix the catheter in position.

For example, the peritoneal catheter may be a resilient, foldable, T-shaped housing chamber with access ports that have elongated, flexible, fluid channels that gather or distribute a liquid such as dialysis fluid. See, e.g., U.S. Pat. No. 5,322,519. The peritoneal catheter may be composed of two linearly mated inflow and outflow conduits contoured as a circular cross-section, which join fluted fluid transport branches. See, e.g., U.S. Pat. No. 6,659,134. The peritoneal catheter may be composed of a ductwork of multiple tubes with fluid holes enclosed within a fluid permeable envelope structure that has slits to allow fluid flow but not tissue adherence. See, e.g., U.S. Pat. No. 5,254,084. The peritoneal catheter may have a one-half helical turn to provide a radial flow and be composed of a plurality of ingress and egress ports positioned about its circumference and length, and have a coating of ultra low temperature isotropic carbon on the intra-abdominal section. See, e.g., U.S. Pat. No. 5,098,413. The peritoneal catheter may be an elongated flexible tube with one end connected to a pair of spaced apart sheets that extends exteriorly into the body cavity with at least one cuff for preventing catheter infections. See, e.g., U.S. Pat. No. 4,368,737. The peritoneal catheter may be composed of two sections which includes a retainer section that permanently ingrows into the abdominal wall and an elongated flexible tube section for delivering and withdrawing dialysate. See, e.g., U.S. Pat. No. 4,278,092. The peritoneal catheter may be flexible tube having a natural bent segment between the proximal and distal ends which includes a flange extending circumferentially at a nonperpendicular angle relative to the axis of the catheter tube. See, e.g., U.S. Pat. No. 4,687,471. The peritoneal catheter may be a percutaneous access device composed of a cylindrical neck portion for skin protrusion, an annular skirt portion for anchoring into the dermis/subcutaneous tissue, and a catheter tube that may be threaded through the neck and skirt portions that has flexible bellows which can form a 90 degree angle. See, e.g., U.S. Pat. No. 4,886,502. The peritoneal catheter may be a flexible, elongated tube with perforations in the wall to pass fluid with a means for urging the central portion of the tube into a tightly wound cylindrical helix configuration. See, e.g., U.S. Pat. No. 4,681,570. Other examples of peritoneal catheters used for dialysis are described in, e.g., U.S. Pat. Nos. 6,290,669; 5,752,939 and 5,171,227.

In another aspect, the peritoneal catheter may be used to administer drugs to the peritoneum. For example, the peritoneal catheter may be a subcutaneous injection catheter apparatus having a receiving chamber with a penetrable membrane to accommodate an injection needle, which may be interconnected to the peritoneal cavity by a hollow stem. See, e.g., U.S. Pat. No. 4,400,169. The peritoneal catheter may be composed of a porous outer casing defining an inner space with an inlet and outlet catheter of non-porous material which are in communication with an opening of the outer casing to form two passageways. See, e.g., U.S. Pat. No. 5,100,392.

Long-term use of peritoneal catheters may lead to infections or blockage of the catheter due to fibrin formation. Synthetic peritoneal catheters and delivery devices that include an anti-scarring drug are capable of preventing stenosis.

Peritoneal catheters, which may be combined with one or more agents according to the present invention, include commercially available products. For example, Cook Critical Care (Bloomington, Ind.) sells the Spiral Chronic Peritoneal Dialysis Catheters and Tenckhoff Chronic Peritoneal Dialysis Catheters. Bard Access Systems (Salt Lake City, Utah) sells the Tenckhoff and HEMOSPLIT Peritoneal Dialysis Catheters. CardioMed Supplies, Inc (ON, Canada) sells the Single Cuff and Double Cuff Straight Peritoneal Dialysis Catheters, as well as the Single Cuff and Double Cuff Coiled Peritoneal Dialysis Catheters. Other companies that sell Single and Double Cuff, Straight and Coiled Tenckhoff catheters and other types of peritoneal catheters include Baxter International, Inc. (Deerfield, Ill.), Fresenius Medical Care (Lexington, Mass.) and Gambro AB (Sweden).

In one aspect, the present invention provides peritoneal access catheters that include an anti-scarring agent or a composition that includes an anti-scarring agent. Numerous polymeric and non-polymeric delivery systems for use in peritoneal dialysis implants and catheters have been described above.

Methods for incorporating the fibrosis-inhibiting agent into or onto the device includes: (a) directly affixing to the device a fibrosis-inhibiting composition (e.g., by either a spraying process or dipping process as described above, with or without a carrier), (b) directly incorporating into the device a fibrosis-inhibiting composition (e.g., by either a spraying process or dipping process as described above, with or without a carrier), (c) by coating the device with a substance such as a hydrogel which will in turn absorb the fibrosis-inhibiting composition, (d) by interweaving fibrosis-inhibiting composition coated thread (or the polymer itself formed into a thread) into the device structure, (e) constructing the device itself or a portion of the device with a fibrosis-inhibiting composition, or (f) by covalently binding the fibrosis-inhibiting agent directly to the device surface or to a linker (small molecule or polymer) that is coated or attached to the device surface. The coatings can be applied to different portions of the device. For example, the coating can be (a) as a coating applied to the external surface of the graft; (b) as a coating applied to the internal (luminal) surface of the graft; (c) as a coating applied to the superficial cuff; (d) as a coating applied to the deep cuff; or (e) as a coating applied to a combination of these surfaces.

The fibrosis-inhibiting agent can be mixed with the materials that are used to make the device such that the fibrosis-inhibiting agent is incorporated into the final device.

In addition to incorporation of a fibrosis-inhibiting agent into or onto the device, another biologically active agent can be incorporated into or onto the device, for example an anti-inflammatory (e.g., dexamethazone or aspirin), antithrombotic agents (e.g., heparin, heparin complexes, hydrophobic heparin derivatives, aspirin, or dipyridamole) and/or an antibiotic including sulfonamides, penicillins, cephalosporins, aminoglycosides (e.g., amoxicillin, trimethoprim-sulfamethoxazole, azithromycin, clarithromycin, bacitracin, polymixin, chloramphenicol, erythromycin, clindomycin, amoxicillin-clavulanate, cefprozil, cefuroxime, cefpodoxime, or cefdinir).

According to the present invention, any fibrosis-inhibiting agent described above can be utilized in the practice of this embodiment. Within one embodiment of the invention, peritoneal dialysis implants and catheters may be adapted to release an agent that inhibits one or more of the four general components of the process of fibrosis (or scarring), including: formation of new blood vessels (angiogenesis), migration and proliferation of connective tissue cells (such as fibroblasts or smooth muscle cells), deposition of extracellular matrix (ECM), and remodeling (maturation and organization of the fibrous tissue). By inhibiting one or more of the components of fibrosis (or scarring), the overgrowth of granulation tissue may be inhibited or reduced.

As peritoneal access catheters devices are made in a variety of configurations and sizes, the exact dose administered will vary with device size, surface area and design. However, certain principles can be applied in the application of this art. Drug dose can be calculated as a function of dose per unit area (of the portion of the device being coated), total dose administered, and appropriate surface concentrations of active drug can be determined. Drugs are to be used at concentrations that range from several times more than to 10%, 5%, or even less than 1% of the concentration typically used in a single chemotherapeutic systemic dose application. Preferably, the drug is released in effective concentrations for a period ranging from 1-90 days.

Preferred fibrosis-inhibiting agents for use in peritoneal access catheters and implants include the following: cell cycle inhibitors including (A) anthracyclines (e.g., doxorubicin and mitoxantrone), (B) taxanes (e.g., paclitaxel, TAXOTERE and docetaxel), and (C) podophyllotoxins (e.g., etoposide); (D) immunomodulators (e.g., sirolimus, everolimus, tacrolimus); (E) heat shock protein 90 antagonists (e.g., geldanamycin); (F) HMGCoA reductase inhibitors (e.g., simvastatin); (G) inosine monophosphate dehydrogenase inhibitors (e.g., mycophenolic acid, 1-alpha-25 dihydroxy vitamin D₃); (H)NF kappa B inhibitors (e.g., Bay 11-7082); (I) antimycotic agents (e.g., sulconizole), (J) p38 MAP kinase inhibitors (e.g., SB202190), and (K) and anti-angiogenesis agents (e.g., halofuginone bromide), as well as analogues and derivatives of the aforementioned.

Regardless of the method of application of the drug to the device, the exemplary anti-fibrosing agents, used alone or in combination, should be administered under the following dosing guidelines. The total amount (dose) of anti-scarring agent in or on the device may be in the range of about 0.01 μg-10 μg, or 10 μg-10 mg, or 10 mg-250 mg, or 250 mg-1000 mg, or 1000 mg-2500 mg. The dose (amount) of anti-scarring agent per unit area of device surface to which the agent is applied may be in the range of about 0.01 μg/mm²-1 μg/mm², or 1 μg/mm²-10 μg/mm², or 10 μg/mm²-250 μg/mm², 250 μg/mm²-1000 μg/mm², or 1000 μg/mm²-2500 μg/mm².

Provided below are exemplary dosage ranges for various anti-scarring agents that can be used in conjunction with peritoneal access catheter devices and implants in accordance with the invention. A) Cell cycle inhibitors including doxorubicin and mitoxantrone. Doxorubicin analogues and derivatives thereof: total dose not to exceed 25 mg (range of 0.1 μg to 25 mg); preferred 1 μg to 5 mg. The dose per unit area of 0.01 μg-100 μg per mm²; preferred dose of 0.1 μg/mm²-10 μg/mm². Minimum concentration of 10⁻⁸-10⁻⁴ M of doxorubicin is to be maintained on the device surface. Mitoxantrone and analogues and derivatives thereof: total dose not to exceed 5 mg (range of 0.01 μg to 5 mg); preferred 0.1 μg to 1 mg. The dose per unit area of the device of 0.01 μg-20 μg per mm²; preferred dose of 0.05 μg/mm²-3 μg/mm². Minimum concentration of 10⁻⁸-10⁻⁴ M of mitoxantrone is to be maintained on the device surface. B) Cell cycle inhibitors including Paclitaxel and analogues and derivatives (e.g., docetaxel) thereof: total dose not to exceed 10 mg (range of 0.1 μg to 10 mg); preferred 1 μg to 3 mg. The dose per unit area of the device of 0.1 μg-10 μg per mm²; preferred dose of 0.25 μg/mm²-5 μg/mm². Minimum concentration of 10⁻⁸-10⁻⁴ M of paclitaxel is to be maintained on the device surface. (C) Cell cycle inhibitors such as podophyllotoxins (e.g., etoposide): total dose not to exceed 10 mg (range of 0.1 μg to 10 mg); preferred 1 μg to 3 mg. The dose per unit area of the device of 0.1 μg-10 μg per mm²; preferred dose of 0.25 μg/mm²-5 μg/mm². Minimum concentration of 10⁻⁸-10⁻⁴ M of etoposide is to be maintained on the device surface. (D) Immunomodulators including sirolimus and everolimus. Sirolimus (i.e., rapamycin, RAPAMUNE): Total dose not to exceed 10 mg (range of 0.1 μg to 10 mg); preferred 10 μg to 1 mg. The dose per unit area of 0.1 μg-100 μg per mm²; preferred dose of 0.5 μg/mm²-10 μg/mm². Minimum concentration of 10⁻⁸-10⁻⁴ M is to be maintained on the device surface. Everolimus and derivatives and analogues thereof: Total dose should not exceed 10 mg (range of 0.1 μg to 10 mg); preferred 10 μg to 1 mg. The dose per unit area of 0.1 μg-100 μg per mm² of surface area; preferred dose of 0.3 μg/mm²-10 μg/mm². Minimum concentration of 10⁻⁸-10⁻⁴ M of everolimus is to be maintained on the device surface. (E) Heat shock protein 90 antagonists (e.g., geldanamycin) and analogues and derivatives thereof: total dose not to exceed 20 mg (range of 0.1 μg to 20 mg); preferred 1 μg to 5 mg. The dose per unit area of the device of 0.1 μg-10 μg per mm²; preferred dose of 0.25 μg/mm²-5 μg/mm². Minimum concentration of 10⁻⁸-10⁻⁴ M of geldanamycin is to be maintained on the device surface. (F) HMGCoA reductase inhibitors (e.g., simvastatin) and analogues and derivatives thereof: total dose not to exceed 2000 mg (range of 10.0 μg to 2000 mg); preferred 10 μg to 300 mg. The dose per unit area of the device of 1.0 μg-1000 μg per mm²; preferred dose of 2.5 μg/mm²-500 μg/mm². Minimum concentration of 10⁻⁸-10⁻³ M of simvastatin is to be maintained on the device surface. (G) Inosine monophosphate dehydrogenase inhibitors (e.g., mycophenolic acid, 1-alpha-25 dihydroxy vitamin D₃) and analogues and derivatives thereof: total dose not to exceed 2000 mg (range of 10.0 μg to 2000 mg); preferred 10 μg to 300 mg. The dose per unit area of the device of 1.0 μg-1000 μg per mm²; preferred dose of 2.5 μg/mm²-500 μg/mm². Minimum concentration of 10⁻⁸-10⁻⁴ M of mycophenolic acid is to be maintained on the device surface. (H) NF kappa B inhibitors (e.g., Bay 11-7082) and analogues and derivatives thereof: total dose not to exceed 200 mg (range of 1.0 μg to 200 mg); preferred 1 μg to 50 mg. The dose per unit area of the device of 1.0 μg-100 μg per mm²; preferred dose of 2.5 μg/mm²-50 μg/mm². Minimum concentration of 10⁻⁸-10⁻⁴ M of Bay 11-7082 is to be maintained on the device surface. (I) Antimycotic agents (e.g., sulconizole) and analogues and derivatives thereof: total dose not to exceed 2000 mg (range of 10.0 μg to 2000 mg); preferred 10 μg to 300 mg. The dose per unit area of the device of 1.0 μg-1000 μg per mm²; preferred dose of 2.5 μg/mm²-500 μg/mm². Minimum concentration of 10⁻⁸-10⁻³ M of sulconizole is to be maintained on the device surface. (J) p38 MAP kinase inhibitors (e.g., SB202190) and analogues and derivatives thereof: total dose not to exceed 2000 mg (range of 10.0 μg to 2000 mg); preferred 10 μg to 300 mg. The dose per unit area of the device of 1.0 μg-1000 μg per mm²; preferred dose of 2.5 μg/mm²-500 μg/mm². Minimum concentration of 10⁻⁸-10⁻³ M of SB202190 is to be maintained on the device surface. (K) Antiangiogenic agents (e.g., halofuginone bromide) and analogues and derivatives thereof: total dose not to exceed 10 mg (range of 0.1 μg to 10 mg); preferred 1 μg to 3 mg. The dose per unit area of the device of 0.1 μg-10 μg per mm²; preferred dose of 0.25 μg/mm²-5 μg/mm². Minimum concentration of 10⁻⁸-10⁻⁴ M of halofuginone bromide is to be maintained on the device surface.

In addition to those described above (e.g., sirolimus, everolimus, and tacrolimus), several other examples of immunomodulators and appropriate dosages ranges for use with peritoneal dialysis catheter devices include the following: (A) Biolimus and derivatives and analogues thereof: Total dose should not exceed 10 mg (range of 0.1 μg to 10 mg); preferred 10 μg to 1 mg. The dose per unit area of 0.1 μg-100 μg per mm² of surface area; preferred dose of 0.3 μg/mm²-10 μg/mm². Minimum concentration of 10⁻⁸-10⁻⁴ M of everolimus is to be maintained on the device surface. (B) Tresperimus and derivatives and analogues thereof: Total dose should not exceed 10 mg (range of 0.1 μg to 10 mg); preferred 10 μg to 1 mg. The dose per unit area of 0.1 μg-100 μg per mm² of surface area; preferred dose of 0.3 μg/mm²-10 μg/mm². Minimum concentration of 10⁻⁸-10⁻⁴ M of tresperimus is to be maintained on the device surface. (C) Auranofin and derivatives and analogues thereof: Total dose should not exceed 10 mg (range of 0.1 μg to 10 mg); preferred 10 μg to 1 mg. The dose per unit area of 0.1 μg-100 μg per mm² of surface area; preferred dose of 0.3 μg/mm²-10 μg/mm². Minimum concentration of 10⁻⁸-10⁻⁴ M of auranofin is to be maintained on the device surface. (D) 27-0-Demethylrapamycin and derivatives and analogues thereof: Total dose should not exceed 10 mg (range of 0.1 μg to 10 mg); preferred 10 μg to 1 mg. The dose per unit area of 0.1 μg-100 μg per mm² of surface area; preferred dose of 0.3 μg/mm²-10 μg/mm². Minimum concentration of 10⁻⁸-10⁻⁴ M of 27-0-demethylrapamycin is to be maintained on the device surface. (E) Gusperimus and derivatives and analogues thereof: Total dose should not exceed 10 mg (range of 0.1 μg to 10 mg); preferred 10 μg to 1 mg. The dose per unit area of 0.1 μg-100 μg per mm² of surface area; preferred dose of 0.3 μg/mm²-10 μg/mm². Minimum concentration of 10⁻⁸-10⁻⁴ M of gusperimus is to be maintained on the device surface. (F) Pimecrolimus and derivatives and analogues thereof: Total dose should not exceed 10 mg (range of 0.1 μg to 10 mg); preferred 10 μg to 1 mg. The dose per unit area of 0.1 μg-100 μg per mm² of surface area; preferred dose of 0.3 μg/mm²-10 μg/mm². Minimum concentration of 10⁻⁸-10⁻⁴ M of pimecrolimus is to be maintained on the device surface and (G) ABT-578 and analogues and derivatives thereof: Total dose should not exceed 10 mg (range of 0.1 μg to 10 mg); preferred 10 μg to 1 mg. The dose per unit area of 0.1 μg-100 μg per mm² of surface area; preferred dose of 0.3 μg/mm²-10 μg/mm². Minimum concentration of 10⁻⁸-10⁻⁴ M of ABT-578 is to be maintained on the device surface.

In addition to those described above (e.g., paclitaxel, TAXOTERE, and docetaxel), several other examples of anti-microtubule agents and appropriate dosages ranges for use with peritoneal dialysis catheter devices include vinca alkaloids such as vinblastine and vincristine sulfate and analogues and derivatives thereof: total dose not to exceed 10 mg (range of 0.1 μg to 10 mg); preferred 1 μg to 3 mg. Dose per unit area of the device of 0.1 μg-10 μg per mm²; preferred dose of 0.25 μg/mm²-5 μg/mm². Minimum concentration of 10⁻⁸-10⁻⁴ M of drug is to be maintained on the device surface.

Central Nervous System Shunts and Pressure Monitoring Devices

In one aspect, the present invention provides for the combination of an anti-scarring agent and a central nervous system (CNS) device, such as a CNS shunt or a pressure monitoring device. CNS devices that comprise an anti-scarring agent are capable of preventing stenosis and obstruction of the device leading to hydrocephalus and increased intercranial pressure.

Hydocephalus, or accumulation of cerebrospinal fluid (CSF) in the brain, is a frequently encountered neurosurgical condition arising from congenital malformations, infection, hemmorrhage, or malignancy. The incompressible fluid exerts pressure on the brain leading to brain damage or even death if untreated. CNS shunts are conduits placed in the ventricles of the brain to divert the flow of CSF from the brain to other body compartments and relieve the fluid pressure. Ventricular CSF is diverted via a prosthetic shunt to a number of drainage locations including the pleura (ventriculopleural shunt), jugular vein, vena cava (VA shunt), gallbladder and peritoneum (VP shunt; most common).

Representative examples of CNS devices include, e.g., CNS shunts, such as ventriculopleural shunts, jugular vein and vena cava (VA) shunts, and ventriculoperitoneal shunt (VP shunt), such as gallbladder and peritoneum shunts; External Ventricular Drainage (EVD) devices; and Intracranial Pressure (ICP) Monitoring Devices. Other CNS devices include, e.g., dural patches and implants to prevent epidural fibrosis post-laminectomy; and devices for continuous subarachnoid infusions.

In one aspect, the CNS device may be a drainage shunt used to drain fluids in the brain. For example, the CNS device may be a cerebrospinal shunt composed of two tubes whereby an inner tube supplies the fluid from the brain ventricles to the peritoneum region and an outer tube is arranged to exert pressure on the inner tube as the volume of fluid builds in the outer tube. See, e.g., U.S. Pat. No. 5,405,316. The CNS device may be a ventricular drainage system adapted for connection to a ventricular drainage catheter for receiving cerebrospinal fluid and having a valve for controlling fluid flow therethrough. See, e.g., U.S. Pat. No. 5,772,625. The CNS device may be a brain ventricular shunt system composed of a brain check valve for preventing cerebrospinal fluid backflow and a flow-rate switching mechanism to provide flow of cerebrospinal fluid from the brain ventricle catheter to the peritoneum or auricle catheter. See, e.g., U.S. Pat. No. 4,781,673. The CNS device may be shunt member with a flow restricting passage that is connected to catheters to provide cerebrospinal fluid drainage from the brain ventricle to the sinus sagittalis. See, e.g., U.S. Pat. No. 6,283,934. The CNS device may be a ventricular end of a ventriculo-cardiac shunt that has a closed distal end with lateral passageways adjacent thereto which are porous and expansible for providing an umbrella-like liner to allow passage of fluid while preventing obstruction. See, e.g., U.S. Pat. No. 3,690,323. The CNS device may be a hydrocephalus valve composed of a chamber with an inlet and outlet valve for routing cerebrospinal fluid away from the brain at a controlled pressure. See, e.g., U.S. Pat. No. 5,069,663. The CNS device may be a hydrocephalus device composed of an external, flexible shell forming a fluid reservoir and housing a non-obstructive, self-regulating valve having a folded membrane which forms a slit-like opening, which has inlet and outlet tubes. See, e.g., U.S. Pat. No. 5,728,061. The CNS device may be a cerebral spinal fluid draining shunt composed of an implantable master control unit that interconnects a cerebral spinal space catheter with a catheter that drains the fluid into a body cavity. See, e.g., U.S. Pat. No. 6,585,677. The CNS device may be a cerebrospinal fluid shunt composed of a ventricular catheter connected to a flexible drainage tube which has an exterior flexible tubular cover from which the drainage tube may be drawn. See, e.g., U.S. Pat. No. 4,950,232. The CNS device may be an intracranial shunting tube composed of a thin film that extends radially and outwardly from the open end of a ventricular tube which has a plurality of side holes to bypass ventricular cerebrospinal fluid to the subdural space on the surface of the brain. See, e.g., U.S. Pat. No. 5,000,731. Other CNS shunts are described in, e.g., U.S. Pat. Nos. 6,575,928; 5,437,626 and 4,631,051.

In another aspect, the CNS device may be a pressure monitoring device. For example, the pressure monitoring device may be an intracranial pressure sensor which is mounted within the skull of a body at the situs where the pressure is to be monitored and a means of transmitting the pressure externally from the skull. See, e.g., U.S. Pat. No. 4,003,141. The pressure monitoring device may be a telemetric differential pressure sensitive device composed of a thin, planar, closed, conductive loop which moves with a flexible diaphragm upon changes in the difference of two bodily pressures on its opposite sides. See, e.g., U.S. Pat. No. 4,593,703. The pressure monitoring device may be composed of a radio-opaque liquid contained within a resiliently compressible vessel of a silastic material in which the volume of liquid is variable as a function of the pressure or force applied to the vessel. See, e.g., U.S. Pat. No. 3,877,137. The pressure monitoring device may be a probe composed of a threaded shaft having a lumen and an engaging lock nut, which is inserted through an opening in the scalp and into the subarachnoid space. See, e.g., U.S. Pat. No. 4,600,013. The pressure monitoring device may be composed of an external transceiver unit and an implantable cavity resonator unit having a dielectric-filled cavity with a predetermined resonance frequency for high frequency electromagnetic waves. See, e.g., U.S. Pat. No. 5,873,840. The pressure monitoring device may be an implantable sensor that detects a physiological parameter (e.g., cerebral spinal fluid flow) and then generates, processes, and transmits the signal to an external receiver. See, e.g., U.S. Pat. No. 6,533,733. Other CNS pressure monitoring devices are described in, e.g., U.S. Pat. Nos. 6,248,080 and 6,210,346.

CNS shunts, which may be combined with one or more agents according to the present invention, include commercially available products, such as the Codman HAKIM Programmable Valves from Codman & Shurtleff, Inc. (Raynham, Mass.), a Johnson & Johnson Company. Other examples include the Integra Neuro Sciences (Plainsboro, N.J.) HEYER-SCHULTE Neurosurgical Shunts, HERMETIC CSF Drainage Systems, and OSV II SMART VALVE Systems and the Medtronic, Inc. (Minneapolis, Minn.) Shunt Assemblies, including the STRATA, DELTA, CSF-Snap and CSF-Flow Control Shunt Assemblies.

Pressure Monitoring CNS devices, which may be combined with one or more agents according to the present invention, include commercially available products such as the VENTRIX Pressure Monitoring Kits and CAMINO Micro Ventricular Bolt ICP Monitoring Catheters from Integra Neuro Sciences (Plainsboro, N.J.).

In one aspect, the present invention provides CNS devices that include an anti-scarring agent or a composition that includes an anti-scarring agent. Numerous polymeric and non-polymeric delivery systems for use in CNS devices have been described above. Methods for incorporating the fibrosis-inhibiting agent into or onto the device includes: (a) directly affixing to the device a fibrosis-inhibiting composition (e.g., by either a spraying process or dipping process as described above, with or without a carrier), (b) directly incorporating into the device a fibrosis-inhibiting composition (e.g., by either a spraying process or dipping process as described above, with or without a carrier), (c) by coating the device with a substance such as a hydrogel which will in turn absorb the fibrosis-inhibiting composition, (d) by interweaving fibrosis-inhibiting composition coated thread (or the polymer itself formed into a thread) into the device structure, (e) constructing the device itself or a portion of the device with a fibrosis-inhibiting composition, or (f) by covalently binding the fibrosis-inhibiting agent directly to the device surface or to a linker (small molecule or polymer) that is coated or attached to the device surface. The coatings can be applied to different portions of the device. For example, the coating can be (a) as a coating applied to the external surface of the shunt; (b) as a coating applied to the internal (luminal) surface of the shunt; or (c) as a coating applied to all or parts of both surfaces The fibrosis-inhibiting agent can be mixed with the materials that are used to make the device such that the fibrosis-inhibiting agent is incorporated into the final device.

In addition to incorporation of a fibrosis-inhibiting agent into or onto the device, another biologically active agent can be incorporated into or onto the device, for example an anti-inflammatory (e.g., dexamethazone or aspirin), antithrombotic agents (e.g., heparin, heparin complexes, hydrophobic heparin derivatives, aspirin, or dipyridamole) and/or an antibiotic (e.g., amoxicillin, trimethoprim-sulfamethoxazole, azithromycin, clarithromycin, amoxicillin-clavulanate, cefprozil, cefuroxime, cefpodoxime, or cefdinir).

According to the present invention, any fibrosis-inhibiting agent described above can be utilized in the practice of this embodiment. Within one embodiment of the invention, CNS devices may be adapted to release an agent that inhibits one or more of the four general components of the process of fibrosis (or scarring), including: formation of new blood vessels (angiogenesis), migration and proliferation of connective tissue cells (such as fibroblasts or smooth muscle cells), deposition of extracellular matrix (ECM), and remodeling (maturation and organization of the fibrous tissue). By inhibiting one or more of the components of fibrosis (or scarring), the overgrowth of granulation tissue may be inhibited or reduced.

As CNS devices are made in a variety of configurations and sizes, the exact dose administered will vary with device size, surface area and design. However, certain principles can be applied in the application of this art. Drug dose can be calculated as a function of dose per unit area (of the portion of the device being coated), total dose administered, and appropriate surface concentrations of active drug can be determined. Drugs are to be used at concentrations that range from several times more than to 10%, 5%, or even less than 1% of the concentration typically used in a single chemotherapeutic systemic dose application. Preferably, the drug is released in effective concentrations for a period ranging from 1-90 days.

Several examples of fibrosis-inhibiting agents for use in CNS devices include the following: cell cycle inhibitors including (A) anthracyclines (e.g., doxorubicin and mitoxantrone), (B) taxanes (e.g., paclitaxel, TAXOTERE and docetaxel), and (C) podophyllotoxins (e.g., etoposide); (D) immunomodulators (e.g., sirolimus, everolimus, tacrolimus); (E) heat shock protein 90 antagonists (e.g., geldanamycin); (F) HMGCoA reductase inhibitors (e.g., simvastatin); (G) inosine monophosphate dehydrogenase inhibitors (e.g., mycophenolic acid, 1-alpha-25 dihydroxy vitamin D₃); (H)NF kappa B inhibitors (e.g., Bay 11-7082); (I) antimycotic agents (e.g., sulconizole), (J) p38 MAP kinase inhibitors (e.g., SB202190), and (K) and anti-angiogenesis agents (e.g., halofuginone bromide), as well as analogues and derivatives of the aforementioned.

Regardless of the method of application of the drug to the device, the exemplary anti-fibrosing agents, used alone or in combination, should be administered under the following dosing guidelines. The total amount (dose) of anti-scarring agent in or on the device may be in the range of about 0.01 μg-10 μg, or 10 μg-10 mg, or 10 mg-250 mg, or 250 mg-1000 mg, or 1000 mg-2500 mg. The dose (amount) of anti-scarring agent per unit area of device surface to which the agent is applied may be in the range of about 0.01 μg/mm²-1 μg/mm², or 1 μg/mm²-10 μg/mm², or 10 μg/mm²-250 μg/mm², 250 μg/mm²-1000 μg/mm², or 1000 μg/mm²-2500 μg/mm².

Provided below are exemplary dosage ranges for various anti-scarring agents that can be used in conjunction with CNS devices in accordance with the invention. A) Cell cycle inhibitors including doxorubicin and mitoxantrone. Doxorubicin analogues and derivatives thereof: total dose not to exceed 25 mg (range of 0.1 μg to 25 mg); preferred 1 μg to 5 mg. The dose per unit area of 0.01 g-100 μg per mm²; preferred dose of 0.1 μg/mm²-10 μg/mm². Minimum concentration of 10⁻⁸-10⁻⁴ M of doxorubicin is to be maintained on the device surface. Mitoxantrone and analogues and derivatives thereof: total dose not to exceed 5 mg (range of 0.01 μg to 5 mg); preferred 0.1 μg to 1 mg. The dose per unit area of the device of 0.01 μg-20 μg per mm²; preferred dose of 0.05 μg/mm²-3 μg/mm². Minimum concentration of 10⁻⁸-10⁻⁴ M of mitoxantrone is to be maintained on the device surface. B) Cell cycle inhibitors including Paclitaxel and analogues and derivatives (e.g., docetaxel) thereof: total dose not to exceed 10 mg (range of 0.1 μg to 10 mg); preferred 1 μg to 3 mg. The dose per unit area of the device of 0.1 μg-10 μg per mm²; preferred dose of 0.25 μg/mm²-5 μg/mm². Minimum concentration of 10⁻⁸-10⁻⁴ M of paclitaxel is to be maintained on the device surface. (C) Cell cycle inhibitors such as podophyllotoxins (e.g., etoposide): total dose not to exceed 10 mg (range of 0.1 μg to 10 mg); preferred 1 μg to 3 mg. The dose per unit area of the device of 0.1 μg-10 μg per mm²; preferred dose of 0.25 μg/mm²-5 μg/mm². Minimum concentration of 10⁻⁸-10⁻⁴ M of etoposide is to be maintained on the device surface. (D) Immunomodulators including sirolimus and everolimus. Sirolimus (i.e., rapamycin, RAPAMUNE): Total dose not to exceed 10 mg (range of 0.1 μg to 10 mg); preferred 10 μg to 1 mg. The dose per unit area of 0.1 μg-100 μg per mm²; preferred dose of 0.5 μg/mm²-10 μg/mm². Minimum concentration of 10⁻⁸-10⁻⁴ M is to be maintained on the device surface. Everolimus and derivatives and analogues thereof: Total dose should not exceed 10 mg (range of 0.1 μg to 10 mg); preferred 10 μg to 1 mg. The dose per unit area of 0.1 μg-100 μg per mm² of surface area; preferred dose of 0.3 μg/mm²-10 μg/mm². Minimum concentration of 10⁻⁸-10⁻⁴ M of everolimus is to be maintained on the device surface. (E) Heat shock protein 90 antagonists (e.g., geldanamycin) and analogues and derivatives thereof: total dose not to exceed 20 mg (range of 0.1 μg to 20 mg); preferred 1 μg to 5 mg. The dose per unit area of the device of 0.1 μg-10 μg per mm²; preferred dose of 0.25 μg/mm²-5 μg/mm². Minimum concentration of 10⁻⁸-10⁻⁴ M of geldanamycin is to be maintained on the device surface. (F) HMGCoA reductase inhibitors (e.g., simvastatin) and analogues and derivatives thereof: total dose not to exceed 2000 mg (range of 10.0 μg to 2000 mg); preferred 10 μg to 300 mg. The dose per unit area of the device of 1.0 μg-1000 μg per mm²; preferred dose of 2.5 μg/mm²-500 μg/mm². Minimum concentration of 10⁻⁸-10⁻³ M of simvastatin is to be maintained on the device surface. (G) Inosine monophosphate dehydrogenase inhibitors (e.g., mycophenolic acid, 1-alpha-25 dihydroxy vitamin D₃) and analogues and derivatives thereof: total dose not to exceed 2000 mg (range of 10.0 μg to 2000 mg); preferred 10 μg to 300 mg. The dose per unit area of the device of 1.0 μg-1000 μg per mm²; preferred dose of 2.5 μg/mm²-500 μg/mm². Minimum concentration of 10⁻⁸-10⁻³ M of mycophenolic acid is to be maintained on the device surface. (H)NF kappa B inhibitors (e.g., Bay 11-7082) and analogues and derivatives thereof: total dose not to exceed 200 mg (range of 1.0 μg to 200 mg); preferred 1 μg to 50 mg. The dose per unit area of the device of 1.0 μg-100 μg per mm²; preferred dose of 2.5 μg/mm²-50 μg/mm². Minimum concentration of 10⁻⁸-10⁻⁴ M of Bay 11-7082 is to be maintained on the device surface. (I) Antimycotic agents (e.g., sulconizole) and analogues and derivatives thereof: total dose not to exceed 2000 mg (range of 10.0 μg to 2000 mg); preferred 10 μg to 300 mg. The dose per unit area of the device of 1.0 μg-1000 μg per mm²; preferred dose of 2.5 μg/mm²-500 μg/mm². Minimum concentration of 10⁻⁸-10⁻³ M of sulconizole is to be maintained on the device surface. (J) p38 MAP kinase inhibitors (e.g., SB202190) and analogues and derivatives thereof: total dose not to exceed 2000 mg (range of 10.0 μg to 2000 mg); preferred 10 μg to 300 mg. The dose per unit area of the device of 1.0 μg-1000 μg per mm²; preferred dose of 2.5 μg/mm²-500 μg/mm². Minimum concentration of 10⁻⁸-10⁻⁴ M of SB202190 is to be maintained on the device surface. (K) Anti-angiogenic agents (e.g., halofuginone bromide) and analogues and derivatives thereof: total dose not to exceed 10 mg (range of 0.01 μg to 10 mg); preferred 1 μg to 3 mg. The dose per unit area of the device of 0.01 μg-10 μg per mm²; preferred dose of 0.25 μg/mm²-5 μg/mm². Minimum concentration of 10⁻⁸-10⁻⁴ M of halofuginone bromide is to be maintained on the device surface.

In addition to those described above (e.g., sirolimus, everolimus, and tacrolimus), several other examples of immunomodulators and appropriate dosages ranges for use with CNS devices and pressure monitoring devices include the following: (A) Biolimus and derivatives and analogues thereof: Total dose should not exceed 10 mg (range of 0.1 μg to 10 mg); preferred 10 μg to 1 mg. The dose per unit area of 0.1 μg-100 μg per mm² of surface area; preferred dose of 0.3 μg/mm²-10 μg/mm². Minimum concentration of 10⁻⁸-10⁻⁴ M of everolimus is to be maintained on the device surface. (B) Tresperimus and derivatives and analogues thereof: Total dose should not exceed 10 mg (range of 0.1 μg to 10 mg); preferred 10 μg to 1 mg. The dose per unit area of 0.1 μg-100 μg per mm² of surface area; preferred dose of 0.3 μg/mm²-10 μg/mm². Minimum concentration of 10⁻⁸-10⁻⁴ M of tresperimus is to be maintained on the device surface. (C) Auranofin and derivatives and analogues thereof: Total dose should not exceed 10 mg (range of 0.1 μg to 10 mg); preferred 10 μg to 1 mg. The dose per unit area of 0.1 μg-100 μg per mm² of surface area; preferred dose of 0.3 μg/mm²-10 μg/mm². Minimum concentration of 10⁻⁸-10⁻⁴ M of auranofin is to be maintained on the device surface. (D) 27-0-Demethylrapamycin and derivatives and analogues thereof: Total dose should not exceed 10 mg (range of 0.1 μg to 10 mg); preferred 10 μg to 1 mg. The dose per unit area of 0.1 μg-100 μg per mm² of surface area; preferred dose of 0.3 μg/mm²-10 μg/mm². Minimum concentration of 10⁻⁸-10⁻⁴ M of 27-0-Demethylrapamycin is to be maintained on the device surface. (E) Gusperimus and derivatives and analogues thereof: Total dose should not exceed 10 mg (range of 0.1 μg to 10 mg); preferred 10 μg to 1 mg. The dose per unit area of 0.1 μg-100 μg per mm² of surface area; preferred dose of 0.3 μg/mm²-10 μg/mm². Minimum concentration of 10⁻⁸-10⁻⁴ M of gusperimus is to be maintained on the device surface. (F) Pimecrolimus and derivatives and analogues thereof: Total dose should not exceed 10 mg (range of 0.1 μg to 10 mg); preferred 10 μg to 1 mg. The dose per unit area of 0.1 μg-100 μg per mm² of surface area; preferred dose of 0.3 μg/mm²-10 μg/mm². Minimum concentration of 10⁻⁸-10⁻⁴ M of pimecrolimus is to be maintained on the device surface and (G) ABT-578 and analogues and derivatives thereof: Total dose should not exceed 10 mg (range of 0.1 μg to 10 mg); preferred 10 μg to 1 mg. The dose per unit area of 0.1 μg-100 μg per mm² of surface area; preferred dose of 0.3 μg/mm²-10 μg/mm². Minimum concentration of 10⁻⁸-10⁻⁴ M of ABT-578 is to be maintained on the device surface.

In addition to those described above (e.g., paclitaxel, TAXOTERE, and docetaxel), several other examples of anti-microtubule agents and appropriate dosages ranges for use with CNS devices and pressure monitoring devices include vinca alkaloids such as vinblastine and vincristine sulfate and analogues and derivatives thereof: total dose not to exceed 10 mg (range of 0.1 μg to 10 mg); preferred 1 μg to 3 mg. Dose per unit area of the device of 0.1 μg-10 μg per mm²; preferred dose of 0.25 μg/mm²-5 μg/mm². Minimum concentration of 10⁻⁸-10⁻⁴ M of drug is to be maintained on the device surface.

Inferior Vena Cava Filters

In one aspect, the present invention provides for the combination of an anti-scarring agent and an inferior vena cava filter device. The term inferior vena cava filters are devices that are intended to capture emboli and prevent them from migrating through the blood stream. Examples of inferior vena cava filters include, without limitation, vascular filters, blood filters, implantable blood filters, caval filters, vena cava filters, vena cava filtering devices, thrombosis filters, thrombus filters, antimigration filters, filtering devices, percutaneous filter systems, intravascular traps, intravascular filters, clot filters, vein filters and body vessel filters.

Inferior vena cava filters catch blood clots to prevent them from traveling to other parts of the body to form an embolus. It may be life threatening if plaques or blood clots migrate through the blood stream and travel to the lungs and cause a pulmonary embolism. To prevent such an occurrence, inferior vena cava filters are placed in the large veins of the body to prevent pulmonary emboli in patients with (or at risk of developing) deep vein thrombosis. Most often these filters are composed of synthetic polymers or metals. These filters may be a variety of configurations, including but not limited to, baskets, cones, umbrellas or loops. The shape of the filter must provide adequate trapping ability while allowing sufficient blood flow. Along with the functional shape, filters may also have other design features including peripheral loops for alignment or anchoring features to prevent migration (e.g., ridges, struts or sharp points). Where the filter comes into contact with the vessel wall for anchoring, a fibrotic response may occur. This fibrotic response can result in difficulties in removal of the filter. This is a particular problem for filters that are to be kept in place for a relatively short period of time. Incorporation of a fibrosis-inhibiting agent into or onto the filter may reduce or prevent stenosis or obstruction of the device via a fibroproliferative response.

In one aspect, inferior vena cava filters may be designed in a variety of configurations. For example, the inferior vena cava filter may be composed of a plurality of intraluminal filter elements held by a retainer in a filter configuration that may be released to an open, stent-like configuration. See, e.g., U.S. Pat. No. 6,267,776. The inferior vena cava filter may be composed of an embolus capturing portion having a plurality of elongated filter wires diverging in a helical arrangement to form a conical surface and an anchoring portion that has a plurality of struts. See, e.g., U.S. Pat. No. 6,391,045. The inferior vena cava filter may be composed of a textured echogenic feature so the filter position may be determined by sonographic visualization. See, e.g., U.S. Pat. No. 6,436,120. The inferior vena cava filter may be composed of a plurality of core wire struts that are anchored to radiate outwardly which are interconnected by compression material to form a filter basket. See, e.g., U.S. Pat. No. 5,370,657. The inferior vena cava filter may be composed of an apical head with a plurality of divergent legs in a conical shaped geometry which have a hook and pad for securing to the vessel. See, e.g., U.S. Pat. No. 5,059,205. The inferior vena cava filter may be composed of a filtering device made of shape memory/superelastic material formed at the distal end of a deployment/retrieval wire section for minimally invasive positioning. See, e.g., U.S. Pat. No. 5,893,869. The inferior vena cava filter may be composed of a plurality of intraluminal elements joined by a retainer, whereby upon release of the retainer, the intraluminal filter elements convert to an open configuration in the blood vessel. See, e.g., U.S. Pat. Nos. 6,517,559 and 6,267,776. The inferior vena cava filter may be composed of an outer catheter and an inner catheter having a collapsible mesh-like filter basket at the distal end made of spring wires or plastic monofilaments. See, e.g., U.S. Pat. No. 5,549,626. The inferior vena cava filter may be composed of a plurality of radiating struts that attach at a body element and has a two layer surface treatment to provide endothelial cell growth and anti-proliferative properties. See, e.g., U.S. Pat. No. 6,273,901. The inferior vena cava filter may be composed of a metal fabric that is configured as a particle-trapping screen that may be slideable along a guidewire. See, e.g., U.S. Pat. No. 6,605,102. The inferior vena cava filter may be non-permanent with a single high memory coiled wire having a cylindrical and a conical segment. See, e.g., U.S. Pat. No. 6,059,825. Other inferior vena cava filters are described in, e.g., U.S. Pat. Nos. 6,623,506; 6,391,044; 6,231,589; 5,984,947; 5,695,518 and 4,817,600.

Vena cava filters, which may be combined with one or more anti-scarring agents according to the present invention, include commercially available products. Examples of vena cava filters that can benefit from the incorporation of a fibrosis-inhibiting agent include, without limitation, the GÜNTHER TULIP Vena Cava FILTER and the GIANTURCO-ROEHM BIRD'S NEST Filter which are sold by Cook, Inc. (Bloomington, Ind.). C.R. Bard (Murray Hill, N.J.) sells the SIMON-NITINOL FILTER and RECOVERY Filter. Cordis Endovascular which is a subsidiary of Cordis Corporation (Miami Lakes, Fla.) sells the TRAPEASE Permanent Vena Cava Filter. B. Braun Medical Inc. (Bethlehem, Pa.) sells the VENA TECH LP Vena Cava Filter and VENA TECH—LGM Vena Cava Filter. Boston Scientific Corporation (Natick, Mass.) sells the Over-the-Wire GREENFIELD Vena Cava Filter.

In one aspect, the present invention provides inferior vena cava filter devices that include an anti-scarring agent or a composition that includes an anti-scarring agent. Numerous polymeric and non-polymeric delivery systems for use in inferior vena cava filters have been described above. These compositions can further comprise one or more fibrosis-inhibiting agents such that the overgrowth of granulation tissue is inhibited or reduced.

Methods for incorporating the fibrosis-inhibiting agent into or onto the device includes: (a) directly affixing to the device a fibrosis-inhibiting composition (e.g., by either a spraying process or dipping process as described above, with or without a carrier), (b) directly incorporating into the device a fibrosis-inhibiting composition (e.g., by either a spraying process or dipping process as described above, with or without a carrier, (c) by coating the device with a substance such as a hydrogel which will in turn absorb the fibrosis-inhibiting composition, (d) by interweaving fibrosis-inhibiting composition coated thread (or the polymer itself formed into a thread) into the device structure, (e) constructing the device itself or a portion of the device with a fibrosis-inhibiting composition, or (f) by covalently binding the fibrosis-inhibiting agent directly to the device surface or to a linker (small molecule or polymer) that is coated or attached to the device surface. The coatings can be applied to different portions of the device. For example, the coating can be (a) as a coating applied to the entire leg of the filter; (b) as a coating applied to the tips of the filter that come into contact with the blood vessel; and/or (c) as a coating applied to all or parts of the entire filter device.

In addition to coating the device with the fibrosis-inhibiting composition, the fibrosis-inhibiting agent can be mixed with the materials that are used to make the device such that the fibrosis-inhibiting agent is incorporated into the final device.

In addition to incorporation of a fibrosis-inhibiting agent into or onto the device, another biologically active agent can be incorporated into or onto the device, for example an anti-inflammatory (e.g., dexamethazone or aspirin), antithrombotic agents (e.g., heparin, heparin complexes, hydrophobic heparin derivatives, aspirin, or dipyridamole), and/or an antibiotic (e.g., amoxicillin, trimethoprim-sulfamethoxazole, azithromycin, clarithromycin, amoxicillin-clavulanate, cefprozil, cefuroxime, cefpodoxime, or cefdinir).

According to the present invention, any fibrosis-inhibiting agent described above can be utilized in the practice of this embodiment. Within one embodiment of the invention, vena cava filters (e.g., inferior vena cava filters) may be adapted to release an agent that inhibits one or more of the four general components of the process of fibrosis (or scarring), including: formation of new blood vessels (angiogenesis), migration and proliferation of connective tissue cells (such as fibroblasts or smooth muscle cells), deposition of extracellular matrix (ECM), and remodeling (maturation and organization of the fibrous tissue). By inhibiting one or more of the components of fibrosis (or scarring), the overgrowth of granulation tissue may be inhibited or reduced.

Several examples of fibrosis-inhibiting agents for use in vena cava filter devices include the following: cell cycle inhibitors including (A) anthracyclines (e.g., doxorubicin and mitoxantrone), (B) taxanes (e.g., paclitaxel, TAXOTERE and docetaxel), and (C) podophyllotoxins (e.g., etoposide); (D) immunomodulators (e.g., sirolimus, everolimus, tacrolimus); (E) heat shock protein 90 antagonists (e.g., geldanamycin); (F) HMGCoA reductase inhibitors (e.g., simvastatin); (G) inosine monophosphate dehydrogenase inhibitors (e.g., mycophenolic acid, 1-alpha-25 dihydroxy vitamin D₃); (H)NF kappa B inhibitors (e.g., Bay 11-7082); (I) antimycotic agents (e.g., sulconizole), (J) p38 MAP kinase inhibitors (e.g., SB202190), and (K) and anti-angiogenesis agents (e.g., halofuginone bromide), as well as analogues and derivatives of the aforementioned.

As vena cava filter devices are made in a variety of configurations and sizes, the exact dose administered will vary with device size, surface area and design. However, certain principles can be applied in the application of this art. Drug dose can be calculated as a function of dose per unit area (of the portion of the device being coated), total dose administered, and appropriate surface concentrations of active drug can be determined. Drugs are to be used at concentrations that range from several times more than to 10%, 5%, or even less than 1% of the concentration typically used in a single chemotherapeutic systemic dose application. Preferably, the drug is released in effective concentrations for a period ranging from 1-90 days.

Regardless of the method of application of the drug to the device, the exemplary anti-fibrosing agents, used alone or in combination, should be administered under the following dosing guidelines. The total amount (dose) of anti-scarring agent in or on the device may be in the range of about 0.01 μg-10 μg, or 10 μg-10 mg, or 10 mg-250 mg, or 250 mg-000 mg, or 1000 mg-2500 mg. The dose (amount) of anti-scarring agent per unit area of device surface to which the agent is applied may be in the range of about 0.01 μg/mm²-1 μg/mm², or 1 μg/mm²-10 μg/mm², or 10 μg/mm²-250 μg/mm², 250 μg/mm²-1000 μg/mm², or 1000 μg/mm²-2500 μg/mm².

Provided below are exemplary dosage ranges for various anti-scarring agents that can be used in conjunction with vena cava devices in accordance with the invention. A) Cell cycle inhibitors including doxorubicin and mitoxantrone. Doxorubicin analogues and derivatives thereof: total dose not to exceed 25 mg (range of 0.1 μg to 25 mg); preferred 1 μg to 5 mg. The dose per unit area of 0.01 μg-100 μg per mm²; preferred dose of 0.1 μg/mm²-10 μg/mm². Minimum concentration of 10⁻⁸-10⁻⁴ M of doxorubicin is to be maintained on the device surface. Mitoxantrone and analogues and derivatives thereof: total dose not to exceed 5 mg (range of 0.01 μg to 5 mg); preferred 0.1 μg to 1 mg. The dose per unit area of the device of 0.01 μg-20 μg per mm²; preferred dose of 0.05 μg/mm²-3 μg/mm². Minimum concentration of 10⁻⁸-10⁻⁴ M of mitoxantrone is to be maintained on the device surface. B) Cell cycle inhibitors including Paclitaxel and analogues and derivatives (e.g., docetaxel) thereof: total dose not to exceed 10 mg (range of 0.1 μg to 10 mg); preferred 1 μg to 3 mg. The dose per unit area of the device of 0.1 μg-10 μg per mm²; preferred dose of 0.25 μg/mm²-5 μg/mm². Minimum concentration of 10⁻⁸-10⁻⁴ M of paclitaxel is to be maintained on the device surface. (C) Cell cycle inhibitors such as podophyllotoxins (e.g., etoposide): total dose not to exceed 10 mg (range of 0.1 μg to 10 mg); preferred 1 μg to 3 mg. The dose per unit area of the device of 0.1 μg-10 μg per mm²; preferred dose of 0.25 μg/mm²-5 μg/mm². Minimum concentration of 10⁻⁸-10⁻⁴ M of etoposide is to be maintained on the device surface. (D) Immunomodulators including sirolimus and everolimus. Sirolimus (i.e., rapamycin, RAPAMUNE): Total dose not to exceed 10 mg (range of 0.1 μg to 10 mg); preferred 10 μg to 1 mg. The dose per unit area of 0.1 μg-100 μg per mm²; preferred dose of 0.5 μg/mm²-10 μg/mm². Minimum concentration of 10⁻⁸-10⁻⁴ M is to be maintained on the device surface. Everolimus and derivatives and analogues thereof: Total dose should not exceed 10 mg (range of 0.1 μg to 10 mg); preferred 10 μg to 1 mg. The dose per unit area of 0.1 μg-100 μg per mm² of surface area; preferred dose of 0.3 μg/mm²-10 μg/mm². Minimum concentration of 10⁻⁸-10⁻⁴ M of everolimus is to be maintained on the device surface. (E) Heat shock protein 90 antagonists (e.g., geldanamycin) and analogues and derivatives thereof: total dose not to exceed 20 mg (range of 0.1 μg to 20 mg); preferred 1 μg to 5 mg. The dose per unit area of the device of 0.1 μg-10 μg per mm²; preferred dose of 0.25 μg/mm²-5 μg/mm². Minimum concentration of 10⁻⁸-10⁻⁴ M of geldanamycin is to be maintained on the device surface. (F) HMGCoA reductase inhibitors (e.g., simvastatin) and analogues and derivatives thereof: total dose not to exceed 2000 mg (range of 10.0 μg to 2000 mg); preferred 10 μg to 300 mg. The dose per unit area of the device of 1.0 μg-1000 μg per mm²; preferred dose of 2.5 μg/mm²-500 μg/mm². Minimum concentration of 10⁻⁸-10⁻³ M of simvastatin is to be maintained on the device surface. (G) Inosine monophosphate dehydrogenase inhibitors (e.g., mycophenolic acid, 1-alpha-25 dihydroxy vitamin D₃) and analogues and derivatives thereof: total dose not to exceed 2000 mg (range of 10.0 μg to 2000 mg); preferred 10 μg to 300 mg. The dose per unit area of the device of 1.0 μg-1000 μg per mm²; preferred dose of 2.5 μg/mm²-500 μg/mm². Minimum concentration of 10⁻⁸-10⁻³ M of mycophenolic acid is to be maintained on the device surface. (H)NF kappa B inhibitors (e.g., Bay 11-7082) and analogues and derivatives thereof: total dose not to exceed 200 mg (range of 1.0 μg to 200 mg); preferred 1 μg to 50 mg. The dose per unit area of the device of 1.0 μg-100 μg per mm²; preferred dose of 2.5 μg/mm²-50 μg/mm². Minimum concentration of 10⁻⁸-10⁻⁴ M of Bay 11-7082 is to be maintained on the device surface. (I) Antimycotic agents (e.g., sulconizole) and analogues and derivatives thereof: total dose not to exceed 2000 mg (range of 10.0 μg to 2000 mg); preferred 10 μg to 300 mg. The dose per unit area of the device of 1.0 μg-1000 μg per mm²; preferred dose of 2.5 μg/mm²-500 μg/mm². Minimum concentration of 10⁻⁸-10⁻³ M of sulconizole is to be maintained on the device surface. (J) p38 MAP kinase inhibitors (e.g., SB202190) and analogues and derivatives thereof: total dose not to exceed 2000 mg (range of 10.0 μg to 2000 mg); preferred 10 μg to 300 mg. The dose per unit area of the device of 1.0 μg-1000 μg per mm²; preferred dose of 2.5 μg/mm²-500 μg/mm². Minimum concentration of 10⁻⁸-10⁻³ M of SB202190 is to be maintained on the device surface. (K) Anti-angiogenic agents (e.g., halofuginone bromide) and analogues and derivatives thereof: total dose not to exceed 10 mg (range of 0.1 μg to 10 mg); preferred 1 μg to 3 mg. The dose per unit area of the device of 0.1 μg-10 μg per mm²; preferred dose of 0.25 μg/mm²-5 μg/mm². Minimum concentration of 10⁻⁸-10⁻⁴ M of halofuginone bromide is to be maintained on the device surface.

In addition to those described above (e.g., sirolimus, everolimus, and tacrolimus), several other examples of immunomodulators and appropriate dosages ranges for use with inferior vena cava devices include the following: (A) Biolimus and derivatives and analogues thereof: Total dose should not exceed 10 mg (range of 0.1 μg to 10 mg); preferred 10 μg to 1 mg. The dose per unit area of 0.1 μg-100 μg per mm² of surface area; preferred dose of 0.3 μg/mm²-10 μg/mm². Minimum concentration of 10⁻⁸-10⁻⁴ M of everolimus is to be maintained on the device surface. (B) Tresperimus and derivatives and analogues thereof: Total dose should not exceed 10 mg (range of 0.1 μg to 10 mg); preferred 10 μg to 1 mg. The dose per unit area of 0.1 μg-100 μg per mm² of surface area; preferred dose of 0.3 μg/mm²-10 μg/mm². Minimum concentration of 10⁻⁸-10⁻⁴ M of tresperimus is to be maintained on the device surface. (C) Auranofin and derivatives and analogues thereof: Total dose should not exceed 10 mg (range of 0.1 μg to 10 mg); preferred 10 μg to 1 mg. The dose per unit area of 0.1 μg-100 μg per mm² of surface area; preferred dose of 0.3 μg/mm²-10 μg/mm². Minimum concentration of 10⁻⁸-10⁻⁴ M of auranofin is to be maintained on the device surface. (D) 27-0-Demethylrapamycin and derivatives and analogues thereof: Total dose should not exceed 10 mg (range of 0.1 μg to 10 mg); preferred 10 μg to 1 mg. The dose per unit area of 0.1 μg-100 μg per mm² of surface area; preferred dose of 0.3 μg/mm²-10 μg/mm². Minimum concentration of 10⁻⁸-10⁻⁴ M of 27-0-Demethylrapamycin is to be maintained on the device surface. (E) Gusperimus and derivatives and analogues thereof: Total dose should not exceed 10 mg (range of 0.1 μg to 10 mg); preferred 10 μg to 1 mg. The dose per unit area of 0.1 μg-100 μg per mm² of surface area; preferred dose of 0.3 μg/mm²-10 μg/mm². Minimum concentration of 10⁻⁸-10⁻⁴ M of gusperimus is to be maintained on the device surface. (F) Pimecrolimus and derivatives and analogues thereof: Total dose should not exceed 10 mg (range of 0.1 μg to 10 mg); preferred 10 μg to 1 mg. The dose per unit area of 0.1 μg-100 μg per mm² of surface area; preferred dose of 0.3 μg/mm²-10 μg/mm². Minimum concentration of 10⁻⁸-10⁻⁴ M of pimecrolimus is to be maintained on the device surface and (G) ABT-578 and analogues and derivatives thereof: Total dose should not exceed 10 mg (range of 0.1 μg to 10 mg); preferred 10 μg to 1 mg. The dose per unit area of 0.1 μg-100 μg per mm² of surface area; preferred dose of 0.3 μg/mm²-10 μg/mm². Minimum concentration of 10⁻⁸-10⁻⁴ M of ABT-578 is to be maintained on the device surface.

In addition to those described above (e.g., paclitaxel, TAXOTERE, and docetaxel), several other examples of anti-microtubule agents and appropriate dosages ranges for use with vena cava filter devices include vinca alkaloids such as vinblastine and vincristine sulfate and analogues and derivatives thereof: total dose not to exceed 10 mg (range of 0.1 μg to 10 mg); preferred 1 μg to 3 mg. Dose per unit area of the device of 0.1 μg-10 μg per mm²; preferred dose of 0.25 μg/mm²-5 μg/mm². Minimum concentration of 10⁻⁸-10⁻⁴ M of drug is to be maintained on the device surface.

Gastrointestinal Devices

In one aspect, the present invention provides for the combination of an anti-scarring agent and a gastrointestinal (GI) device. There are many gastrointestinal tube devices that are used for feeding applications and for drainage applications. The functioning of these tubes can be compromised if there is an excessive fibroproliferative response to these devices. The incorporation of a fibrosis-inhibiting agent into or onto the device can modulate this fibroproliferative response (e.g., to prevent stenosis and/or obstruction of the device) thereby maintaining performance of the device.

A variety of GI tubes for drainage or feeding can be combined with a fibrosis-inhibiting agent to prevent stenosis and/or obstruction of the device. These devices may include, without limitation, GI tubes for drainage or feeding, portosystemic shunts, shunts for ascites, nasogastric or nasoenteral tubes, gastrostomy or percutaneous feeding tubes, jejunostomy endoscopic tubes, colostomy devices, drainage tubes, biliary T-tubes, biopsy forceps, biliary stone removal devices, endoscopic retrograde cholangiopancretography (ERCP) devices, dilation balloons, enteral feeding devices, stents, low profile devices, virtual colonoscopy (VC) devices, capsule endoscopes, and retrieval devices.

GI devices may be composed of synthetic materials, including, without limitation, stainless steel, metals, nitinol, glass, resins or polymers.

In one aspect, the GI device may be an instrument used to examine or provide access to the interior of the gastrointestinal tract. This may include optical imaging in the form of still imaging or videoing for diagnosing purposes. Procedures that use these devices include, without limitation, enteroscopy, colonoscopy or esophagogastroduodenoscopy, where an endoscope enters the esophagus or anal canal to assess portions of the GI tract. For example, the GI device may be an endoscope having a tubular shaft for receiving a viewing lens and a treatment instrument. See, e.g., U.S. Pat. No. 5,421,323. The GI device may be a multi-lumen endoscopic catheter that may be inserted through an endoscope for the practice of endoscopic retrograde cholangiopancreatography, whereby the first lumen has a wire threaded through it, the second lumen provides a conduit to infuse a radio-opaque contrast medium to identify obstructions, and the third lumen provides a conduit to dilate a balloon. See, e.g., U.S. Pat. Nos. 5,788,681 and 5,843,028. The GI device may be a video endoscope system composed of a swallowable capsule, a transmitter and a reception system. See, e.g., U.S. Pat. No. 5,604,531. The GI device may be an endoscope composed of an encapsulated ultrasonic transducer capsule having a self-contained electromechanical sector scanner, which may be used for transesophageal echocardiography. See, e.g., U.S. Pat. Nos. 4,977,898 and 4,834,102. The GI device may be a sterilizable endoscope having an image sensor mounted on a cylindrical capsule and a separable disposable channel. See, e.g., U.S. Pat. No. 5,643,175. The GI device may be a body canal intrusion instrument that may be composed of a bi-directional surface friction for engaging tissue during navigation to decrease the risk of puncture and time associated with the insertion of catheters, guidewires and endoscopes through body cavities and canals. See, e.g., U.S. Pat. No. 6,589,213. The GI device may be a colonic access device composed of flexible tubing with a tether for releasing from a colonoscope, which may be placed in the colon for up to several days to monitor and treat colorectal diseases. See, e.g., U.S. Pat. No. 6,149,581. The GI device may be adapted for the bile or pancreatic duct by being composed of a mother endoscope that is inserted into the duodenum and a daughter endoscope that is inserted via papilla through a forceps channel. See, e.g., U.S. Pat. No. 4,979,496.

In another aspect, the GI device may be used as a conduit for long-term tube feeding. These GI devices may include, without limitation, percutaneous feeding tubes, enteral feeding devices/catheters, gastrostomy feeding tubes, low profile devices, and nasogastric tubes. These long-term feeding tubes may be advanced through the GI tract via nasal canal or through the abdominal wall via a gastrostomy. For example, the GI device may be an enteral feeding catheter adapted to serve as a conduit for passage of sustenance through an abdominal wall into the body and having a retainer and retractable locking means. See, e.g., U.S. Pat. No. 4,826,481. The GI device may be an enteral feeding tube having a catheter that allows for easy insertion and removal by having a slim, tapered guide tube and a balloon bolster. See, e.g., U.S. Pat. No. 6,582,395. The GI device may be an enteral feeding device for administering fluids into the stomach, which is composed of a female connector, flexible feeding tube, fluid discharge tube, and probe, which are connected to the male end of the guide wire. See, e.g., U.S. Pat. No. 5,242,429. The GI device may be a hollow, cylindrical elongated body with a spring-biased valve, which is maintained through a surgical opening in the stomach wall by an extended concentric flange that facilitates fixation. See, e.g., U.S. Pat. No. 4,344,435. The GI device may be a nasogastric tube having openings along its distal end with a coupled introducer flexible sheath extending longitudinally along the tube. See, e.g., U.S. Pat. No. 5,334,167. Other GI devices used as feeding tubes or related devices are described in, e.g., U.S. Pat. Nos. 6,582,395; 5,989,225; 5,720,734; 5,716,347; 5,503,629; 5,342,321; 4,861,334; 4,758,219 and 4,057,065.

In another aspect, the GI device may be used for irrigation or aspiration of the GI tract. These GI devices may be used, for example, to remove ingested poisons or blood, to treat absorption-related conditions, to decompress the stomach, pre-operatively to ensure portions of the GI tract is empty, post-operatively to remove gas, and to treat diseases such as bowel obstructions or paralytic ileus. For example, the GI tube may be elongated and configured to be inserted in the GI tract having a slidable treatment device for controlling bleeding and a fluid reservoir coupled to the tube. See, e.g., U.S. Pat. No. 5,947,926. The GI tube may be a nasogastric flexible tube with a curved or bent leading end to anatomically conform and facilitate advancement into the esophagus and stomach. See, e.g., U.S. Pat. No. 5,690,620. The GI tube may be a nasogastric elongated tube fixedly bent to extend from the nostril without affixation to avoid pressure necrosis in the nose due to force exertion. See, e.g., U.S. Pat. No. 4,363,323. The GI device may be composed of aspirating, feeding and inflation lumens, which is surgically inserted through the abdominal and gastric wall. See, e.g., U.S. Pat. No. 4,543,089. The GI device may be composed of drain tube and irrigating tube with a cuffed fluid sealing that is used for unidirectional irrigation of the bowels. See, e.g., U.S. Pat. No. 4,637,814. The GI device may be an open-ended, thin-walled, balloon-like tube shaped to extend through at least part of an alimentary canal for the purpose of passing digested food solids and thereby treating absorption-related diseases. See, e.g., U.S. Pat. Nos. 4,315,509 and 4,134,405.

In another aspect, the GI device may be a colostomy device. For example, the colostomy device may be an artificial anus composed of a hollow tubular support with a cylindrical body having a pair of radially-extending flanges to engage the member See, e.g., U.S. Pat. No. 4,781,176. The colostomy device may be composed of internal and external balloons connected by a tube and an annular supporting plate for attachment to the stoma or rectum. See, e.g., U.S. Pat. No. 5,569,216.

In another aspect, the GI device may be a mechanical hemostatic device used to control GI bleeding. Hemostatic devices, which are used to constrict blood flow, may include, without limitation, clamps, clips, staples and sutures. For example, the hemostatic device may be a compression clip composed of an anchor and stem having a transverse hole and a bolster which may be fixed or movable along the stem. See, e.g., U.S. Pat. No. 6,387,114. The hemostatic device may be an endoscopic clip composed of deformable material and a tissue-penetrating pair of hollow jaws. See, e.g., U.S. Pat. No. 5,989,268.

In another aspect, the GI device may be a means to clear blocked GI tracts. For example, the GI device may be a dilation catheter composed of a shroud tube having a strain relief tube extending from within which is used to alter the configuration of a dilation balloon. See, e.g., U.S. Pat. No. 6,537,247.

In another aspect, the GI device may function to deliver drug to the GI tract. For example, the GI device may be orally administered and composed of a two-chambered water-permeable body, in which one chamber has an orifice for expelling a: liquid drug when under pressure, and the second chamber contains an electric circuit that generates a gas which compresses the first chamber to expel the drug. See, e.g., U.S. Pat. No. 5,925,030. The GI device may be a collapsible, ellipsoidal gastric anchor with a tether and a long, narrow intestinal payload module, which contains slow release medicaments, bound enzymes or nonpathogenic microorganisms. See, e.g., U.S. Pat. No. 4,878,905. The GI device may be an ingestible device for delivering a substance to a chosen site within the GI tract, which includes a receiver of electromagnetic radiation for powering an openable part of the device for inserting or dispensing the substance. See, e.g., U.S. Pat. No. 6,632,216.

In another aspect, the GI device may be a shunting device used to provide communication between two bodily systems. Shunting devices may be used to treat abnormal conditions, such as bypassing occlusions in a body passageway or transferring unwanted accumulation of fluids from a body cavity to a site where it can be processed by the body. For example, a shunting device may be used to displace peritoneal cavity fluid into the systemic venous circulation as a treatment for ascites. Shunting devices may include, without limitation, portosystemic shunts and peritoneovenous shunts. For example, the shunt may be an implantable pump composed of a cylindrical chamber and port with pumping means for aspirating fluid and expelling fluids. See, e.g., U.S. Pat. No. 4,725,207. The shunt may be an implantable peritoneovenous shunt system composed of a double-chambered ascites collection device, a pump (e.g., magnetically driven or compression driven), and an anti-reflux catheter, that are all connected by flexible tubing. See, e.g., U.S. Pat. Nos. 4,657,530 and 4,610,658. The shunt may be composed of a peritoneal tube connected to a hollow plastic implanted valve assembly that passes fluid when under pressure to a venous tube. See, e.g., U.S. Pat. No. 5,520,632. The shunt may be a collapsible, shape-memory metal fabric with a plurality of woven metal strands having a central passageway for fluid and delivered in a collapsed state through a body channel to create a portosystemic shunt. See, e.g., U.S. Pat. No. 6,468,303. The GI device may be a laparoscopic tunneling dissector composed of an inflatable balloon and a hollow blunt tipped obturator which is used to tunnel through tissue to provide an anatomic working space for laparoscopic procedures. See, e.g., U.S. Pat. Nos. 5,836,961 and 5,817,123.

GI devices, which may be combined with one or more agents according to the present invention, include commercially available products.

In one aspect, GI devices that are used for feeding purposes may include a variety of devices. For example, gastrostomy tubes such as the DURA-G Polyurethane Gastrostomy Tubes and MAGNA-PORT Gastrostomy Tubes are sold by Ross Products (Columbus, Ohio), a division of Abbott Laboratories. Moss Tubes, Inc. (West Sand Lake, N.Y.) sells the MOSS G-Tube Percutaneous Endoscopic Gastrostomy Kits. Other enteral feeding tubes include, for example, EASY-FEED Enteral Feeding Sets which are sold by Ross Products (Columbus, Ohio), a division of Abbott Laboratories. COMPAT Enteral Delivery Systems are sold by Novartis AG (Basel, Switzerland). CORFLO Feeding Tubes are sold by VIASYS Healthcare Medsystems Division (Wheeling, Ill.). ENDOVIVE Enteral Feeding Systems are sold by Boston Scientific Corporation. Nasogastric tubes, such as the Mark IV Nasal (SIL) Tubes are sold by Moss Tubes, Inc. (West Sand Lake, N.Y.). Bard Medical Division (Covington, Ga.) of C.R. Bard, Inc. and Andersen Products Limited (England, United Kingdom) also sells a variety of Nasogastric Feeding Tubes. Low profile devices, such as the Low-Profile Replacement Gastrostomy Devices and the Bard Button Replacement Gastrostomy Devices are sold by Bard Endoscopic Technologies (Billerica, Mass.), a division of C.R. Bard, Inc.

In another aspect, GI devices may include gastrointestinal tubes for irrigation or aspiration, such as the LAVACUATOR Gastro Intestinal Tubes and VENTROL Levine Tubes, which are sold by Nellcor Puritan Bennett Inc. (Pleasanton, Calif.).

In another aspect, GI devices may include those used as portosystemic shunts or other shunting devices, such as the VIATORR TIPS Endoprostheses that are sold by W.L. Gore & Associates, Inc. (Newark, Del.). Denver Ascites Shunts are sold by Denver Biomedical, Inc. (Golden, Colo.). LEVEEN Shunts are sold by Becton, Dickinson and Company (Franklin Lakes, N.J.).

In another aspect, GI devices may include colostomy devices, such as ASSURA Pouches and COLOPLAST Pouches, which are sold by Coloplast Corporation (Marietta, Ga.). ESTEEM SYNERGY Standard Closed-End Pouches and SUR-FIT NATURA Closed-End Pouches are sold by ConvaTec (Princeton, N.J.), a Bristol-Myers Squibb Company. Cymed Ostomy Company (Berkeley, Calif.) sells the MICROSKIN Colostomy Pouching Systems. KARAYA 5 One-Piece Pouching Systems, CONTOUR I One-Piece Ostomy Pouching Systems, and CENTERPOINTLOCK (CPL) Two-Piece Pouching Systems are sold by Hollister Inc. (Libertyville, Ill.). Bard Medical Division (Covington, Ga.) of C.R. Bard, Inc. also sells a variety of Colostomy Pouches.

In another aspect, GI devices may include dilatation catheters, such as the ELIMINATOR Multi-Stage Balloon Dilators, which are sold by Bard Endoscopic Technologies (Billerica, Mass.), a division of C.R. Bard, Inc. CRE Fixed Wire and Wireguided Balloon Dilators are sold by Boston Scientific Corporation (Natick, Mass.).

In one aspect, the present invention provides GI devices that include an anti-scarring agent or a composition that includes an anti-scarring agent. Numerous polymeric and non-polymeric delivery systems have been described above. These compositions can further comprise one or more fibrosis-inhibiting agents such that the overgrowth of granulation tissue is inhibited or reduced.

Methods for incorporating the fibrosis-inhibiting agent into or onto the device includes: (a) directly affixing to the device a fibrosis-inhibiting composition (e.g., by either a spraying process or dipping process as described above, with or without a carrier), (b) directly incorporating into the device a fibrosis-inhibiting composition (e.g., by either a spraying process or dipping process as described above, with or without a carrier, (c) by coating the device with a substance such as a hydrogel which will in turn absorb the fibrosis-inhibiting composition, (d) by interweaving fibrosis-inhibiting composition coated thread (or the polymer itself formed into a thread) into the device structure, (e) constructing the device itself or a portion of the device with a fibrosis-inhibiting composition, or (f) by covalently binding the fibrosis-inhibiting agent directly to the device surface or to a linker (small molecule or polymer) that is coated or attached to the device surface. The coatings can be applied to different portions of the device. For example, the coating can be (a) as a coating applied to the external surface of the tube; (b) as a coating applied to the internal (luminal) surface of the tube; (c) as a coating applied to the ends of the tube; and/or (d) as a coating applied to all or parts of both surfaces of the tube.

In addition to coating the device with the fibrosis-inhibiting composition, the fibrosis-inhibiting agent can be mixed with the materials that are used to make the device such that the fibrosis-inhibiting agent is incorporated into the final device.

In addition to incorporation of a fibrosis-inhibiting agent into or onto the device, another biologically active agent can be incorporated into or onto the device, for example an anti-inflammatory (e.g., dexamethazone or aspirin), antithrombotic agents (e.g., heparin, heparin complexes, hydrophobic heparin derivatives, aspirin, or dipyridamole) and/or an antibiotic (e.g., amoxicillin, trimethoprim-sulfamethoxazole, azithromycin, clarithromycin, amoxicillin-clavulanate, cefprozil, cefuroxime, cefpodoxime, or cefdinir).

According to the present invention, any anti-scarring agent described above can be utilized in the practice of this embodiment. Within one embodiment of the invention, GI devices may be adapted to release an agent that inhibits one or more of the four general components of the process of fibrosis (or scarring), including: formation of new blood vessels (angiogenesis), migration and proliferation of connective tissue cells (such as fibroblasts or smooth muscle cells), deposition of extracellular matrix (ECM), and remodeling (maturation and organization of the fibrous tissue). By inhibiting one or more of the components of fibrosis (or scarring), the overgrowth of granulation tissue may be inhibited or reduced.

Examples of fibrosis-inhibiting agents for use with GI devices include the following: cell cycle inhibitors including (A) anthracyclines (e.g., doxorubicin and mitoxantrone), (B) taxanes (e.g., paclitaxel, TAXOTERE and docetaxel), and (C) podophyllotoxins (e.g., etoposide); (D) immunomodulators (e.g., sirolimus, everolimus, tacrolimus); (E) heat shock protein 90 antagonists (e.g., geldanamycin); (F) HMGCoA reductase inhibitors (e.g., simvastatin); (G) inosine monophosphate dehydrogenase inhibitors (e.g., mycophenolic acid, 1-alpha-25 dihydroxy vitamin D₃); (H)NF kappa B inhibitors (e.g., Bay 11-7082); (I) antimycotic agents (e.g., sulconizole), (J) p38 MAP kinase inhibitors (e.g., SB202190), and (K) and anti-angiogenesis agents (e.g., halofuginone bromide), as well as analogues and derivatives of the aforementioned.

As GI devices are made in a variety of configurations and sizes, the exact dose administered will vary with device size, surface area and design. However, certain principles can be applied in the application of this art. Drug dose can be calculated as a function of dose per unit area (of the portion of the device being coated), total dose administered, and appropriate surface concentrations of active drug can be determined. Drugs are to be used at concentrations that range from several times more than to 10%, 5%, or even less than 1% of the concentration typically used in a single chemotherapeutic systemic dose application. Preferably, the drug is released in effective concentrations for a period ranging from 1-90 days.

Regardless of the method of application of the drug to the device, the exemplary anti-fibrosing agents, used alone or in combination, should be administered under the following dosing guidelines. The total amount (dose) of anti-scarring agent in or on the device may be in the range of about 0.01 μg-10 μg, or 10 μg-10 mg, or 10 mg-250 mg, or 250 mg-1000 mg, or 1000 μg-2500 mg. The dose (amount) of anti-scarring agent per unit area of device surface to which the agent is applied may be in the range of about 0.01 μg/mm²-1 μg/mm², or 1 μg/mm²-10 μg/mm², or 10 μg/mm²-250 μg/mm², 250 μg/mm²-1000 μg/mm², or 1000 μg/mm²-2500 μg/mm².

Provided below are exemplary dosage ranges for various anti-scarring agents that can be used in conjunction with GI devices in accordance with the invention. A) Cell cycle inhibitors including doxorubicin and mitoxantrone. Doxorubicin analogues and derivatives thereof: total dose not to exceed 25 mg (range of 0.1 μg to 25 mg); preferred 1 μg to 5 mg. The dose per unit area of 0.01 μg-100 μg per mm²; preferred dose of 0.1 μg/mm²-10 μg/mm². Minimum concentration of 10⁻⁸-10⁻⁴ M of doxorubicin is to be maintained on the device surface. Mitoxantrone and analogues and derivatives thereof: total dose not to exceed 5 mg (range of 0.01 μg to 5 mg); preferred 0.1 μg to 1 mg. The dose per unit area of the device of 0.01 μg-20 μg per mm²; preferred dose of 0.05 μg/mm²-3 μg/mm². Minimum concentration of 10⁻⁸-10⁻⁴ M of mitoxantrone is to be maintained on the device surface. B) Cell cycle inhibitors including Paclitaxel and analogues and derivatives (e.g., docetaxel) thereof: total dose not to exceed 10 mg (range of 0.1 μg to 10 mg); preferred 1 μg to 3 mg. The dose per unit area of the device of 0.1 μg-10 μg per mm²; preferred dose of 0.25 μg/mm²-5 μg/mm². Minimum concentration of 10⁻⁸-10⁻⁴ M of paclitaxel is to be maintained on the device surface. (C) Cell cycle inhibitors such as podophyllotoxins (e.g., etoposide): total dose not to exceed 10 mg (range of 0.1 μg to 10 mg); preferred 1 μg to 3 mg. The dose per unit area of the device of 0.1 μg-10 μg per mm²; preferred dose of 0.25 μg/mm²-5 μg/mm². Minimum concentration of 10⁻⁸-10⁻⁴ M of etoposide is to be maintained on the device surface. (D) Immunomodulators including sirolimus and everolimus. Sirolimus (i.e., rapamycin, RAPAMUNE): Total dose not to exceed 10 mg (range of 0.1 μg to 10 mg); preferred 10 μg to 1 mg. The dose per unit area of 0.1 μg-100 μg per mm²; preferred dose of 0.5 μg/mm²-10 μg/mm². Minimum concentration of 10⁻⁸-10⁻⁴ M is to be maintained on the device surface. Everolimus and derivatives and analogues thereof: Total dose should not exceed 10 mg (range of 0.1 μg to 10 mg); preferred 10 μg to 1 mg. The dose per unit area of 0.1 μg-100 μg per mm² of surface area; preferred dose of 0.3 μg/mm²-10 μg/mm². Minimum concentration of 10⁻⁸-10⁻⁴ M of everolimus is to be maintained on the device surface. (E) Heat shock protein 90 antagonists (e.g., geldanamycin) and analogues and derivatives thereof: total dose not to exceed 20 mg (range of 0.1 μg to 20 mg); preferred 1 μg to 5 mg. The dose per unit area of the device of 0.1 μg-10 μg per mm²; preferred dose of 0.25 μg/mm²-5 μg/mm². Minimum concentration of 10⁻⁸-10⁻⁴ M of geldanamycin is to be maintained on the device surface. (F) HMGCoA reductase inhibitors (e.g., simvastatin) and analogues and derivatives thereof: total dose not to exceed 2000 mg (range of 10.0 μg to 2000 mg); preferred 10 μg to 300 mg. The dose per unit area of the device of 1.0 μg-1000 μg per mm²; preferred dose of 2.5 μg/mm²-500 μg/mm². Minimum concentration of 10⁻⁸-10⁻³ M of simvastatin is to be maintained on the device surface. (G) Inosine monophosphate dehydrogenase inhibitors (e.g., mycophenolic acid, 1-alpha-25 dihydroxy vitamin D₃) and analogues and derivatives thereof: total dose not to exceed 2000 mg (range of 10.0 μg to 2000 mg); preferred 10 μg to 300 mg. The dose per unit area of the device of 1.0 μg-1000 μg per mm²; preferred dose of 2.5 μg/mm²-500 μg/mm². Minimum concentration of 10⁻⁸-10⁻³ M of mycophenolic acid is to be maintained on the device surface. (H)NF kappa B inhibitors (e.g., Bay 11-7082) and analogues and derivatives thereof: total dose not to exceed 200 mg (range of 1.0 μg to 200 mg); preferred 1 μg to 50 mg. The dose per unit area of the device of 1.0 μg-100 μg per mm²; preferred dose of 2.5 μg/mm²-50 μg/mm². Minimum concentration of 10⁻⁸-10⁻⁴ M of Bay 11-7082 is to be maintained on the device surface. (I) Antimycotic agents (e.g., sulconizole) and analogues and derivatives thereof: total dose not to exceed 2000 mg (range of 10.0 μg to 2000 mg); preferred 10 μg to 300 mg. The dose per unit area of the device of 1.0 μg-1000 μg per mm²; preferred dose of 2.5 μg/mm²-500 μg/mm². Minimum concentration of 10⁻⁸-10⁻⁴ M of sulconizole is to be maintained on the device surface. (J) p38 MAP kinase inhibitors (e.g., SB202190) and analogues and derivatives thereof: total dose not to exceed 2000 mg (range of 10.0 μg to 2000 mg); preferred 10 μg to 300 mg. The dose per unit area of the device of 1.0 μg-1000 μg per mm²; preferred dose of 2.5 μg/mm²-500 μg/mm². Minimum concentration of 10⁻⁸-10⁻⁴ M of SB202190 is to be maintained on the device surface. (K) Anti-angiogenic agents (e.g., halofuginone bromide) and analogues and derivatives thereof: total dose not to exceed 10 mg (range of 0.1 μg to 10 mg); preferred 1 μg to 3 mg. The dose per unit area of the device of 0.1 μg-10 μg per mm²; preferred dose of 0.25 μg/mm²-5 μg/mm². Minimum concentration of 10⁻⁸-10⁻⁴ M of halofuginone bromide is to be maintained on the device surface.

In addition to those described above (e.g., sirolimus, everolimus, and tacrolimus), several other examples of immunomodulators and appropriate dosages ranges for use with GI devices include the following: (A) Biolimus and derivatives and analogues thereof: Total dose should not exceed 10 mg (range of 0.1 μg to 10 mg); preferred 10 μg to 1 mg. The dose per unit area of 0.1 μg-100 μg per mm² of surface area; preferred dose of 0.3 μg/mm²-10 μg/mm². Minimum concentration of 10⁻⁸-10⁻⁴ M of everolimus is to be maintained on the device surface. (B) Tresperimus and derivatives and analogues thereof: Total dose should not exceed 10 mg (range of 0.1 μg to 10 mg); preferred 10 μg to 1 mg. The dose per unit area of 0.1 μg-100 μg per mm² of surface area; preferred dose of 0.3 μg/mm²-10 μg/mm². Minimum concentration of 10⁻⁸-10⁻⁴ M of tresperimus is to be maintained on the device surface. (C) Auranofin and derivatives and analogues thereof: Total dose should not exceed 10 mg (range of 0.1 μg to 10 mg); preferred 10 μg to 1 mg. The dose per unit area of 0.1 μg-100 μg per mm² of surface area; preferred dose of 0.3 μg/mm²-10 μg/mm². Minimum concentration of 10⁻⁸-10⁻⁴ M of auranofin is to be maintained on the device surface. (D) 27-0-Demethylrapamycin and derivatives and analogues thereof: Total dose should not exceed 10 mg (range of 0.1 μg to 10 mg); preferred 10 μg to 1 mg. The dose per unit area of 0.1 μg-100 μg per mm² of surface area; preferred dose of 0.3 μg/mm²-10 μg/mm². Minimum concentration of 10⁻⁸-10⁻⁴ M of 27-0-Demethylrapamycin is to be maintained on the device surface. (E) Gusperimus and derivatives and analogues thereof: Total dose should not exceed 10 mg (range of 0.1 μg to 10 mg); preferred 10 μg to 1 mg. The dose per unit area of 0.1 μg-100 μg per mm² of surface area; preferred dose of 0.3 μg/mm²-10 μg/mm². Minimum concentration of 10⁻⁸-10⁻⁴ M of gusperimus is to be maintained on the device surface. (F) Pimecrolimus and derivatives and analogues thereof: Total dose should not exceed 10 mg (range of 0.1 μg to 10 mg); preferred 10 μg to 1 mg. The dose per unit area of 0.1 μg-100 μg per mm² of surface area; preferred dose of 0.3 μg/mm²-10 μg/mm². Minimum concentration of 10⁻⁸-10⁻⁴ M of pimecrolimus is to be maintained on the device surface and (G) ABT-578 and analogues and derivatives thereof: Total dose should not exceed 10 mg (range of 0.1 μg to 10 mg); preferred 10 μg to 1 mg. The dose per unit area of 0.1 μg-100 μg per mm² of surface area; preferred dose of 0.3 μg/mm²-10 μg/mm². Minimum concentration of 10⁻⁸-10⁻⁴ M of ABT-578 is to be maintained on the device surface.

In addition to those described above (e.g., paclitaxel, TAXOTERE, and docetaxel), several other examples of anti-microtubule agents and appropriate dosages ranges for use with GI devices include vinca alkaloids such as vinblastine and vincristine sulfate and analogues and derivatives thereof: total dose not to exceed 10 mg (range of 0.1 μg to 10 mg); preferred 1 μg to 3 mg. Dose per unit area of the device of 0.1 μg-10 μg per mm²; preferred dose of 0.25 μg/mm²-5 μg/mm². Minimum concentration of 10⁻⁸-10⁻⁴ M of drug is to be maintained on the device surface.

Central Venous Catheters

In one aspect, the present invention provides for the combination of an anti-scarring agent and a central venous catheter (CVC) device. For the purposes of this invention, the term “Central Venous Catheters” should be understood to include any catheter or line that is used to deliver fluids to the large (central) veins of the body (e.g., jugular, pulmonary, femoral, iliac, inferior vena cava, superior vena cava, axillary etc.). CVC devices are generally hollow, tubular cannulae that are inserted into body passageways to permit injection or withdrawal of bodily fluids. CVCs may be inserted into a large vein, such as the superior vena cava, with a portion of the catheter disposed within the body and a connection port which extends out of the body for access to the circulatory system. CVCs may be used to administer drugs (e.g., chemotherapy or antibiotic therapy) or intravenous feeding, pressure monitoring or periodic blood sampling.

CVCs may be designed with or without a cuff or flange. Cuffs are used to prevent the catheter from slipping or becoming infected. CVCs may have one lumen or multiple lumens and range in many sizes to adapt to the required needs. They may be composed of synthetic materials, including, but not limited to, polyurethane, polyethylene, silicone, copolymers and other polymeric compositions.

CVCs are typically left in the body for a long period of time and thus, may develop infection or inflammation in response to the catheter. CVC access lumens may be blocked by clotted blood or thrombus formation. Some CVCs may also be available with coatings and treated surfaces to minimize the risk of infection and/or inflammation. The incorporation of a fibrosis-inhibiting agent into or onto the device can modulate an excessive fibroproliferative response to the device, which may prevent stenosis and/or obstruction of the device.

In one aspect, the CVC may be designed for specialized access to the circulatory system for specific conditions/purposes. For example, the CVC may be especially made for hemodialysis use by being elongated with a needle-like, dual lumen that may be used as a conduit for administering drugs or additives into the body through an AV access fistula or graft. See, e.g., U.S. Pat. No. 5,876,366. The CVC may be composed of an indwelling cannula adapted for placement within the superior vena cava having an exit port at the distal end whereby fluid medicament may be delivered to essentially the area of subcutaneous tissue surrounding the cannula. See, e.g., U.S. Pat. No. 5,817,072.

In another aspect, the CVC may be designed to provide multiple conduits for accessing the circulatory system. For example, the CVC may be an elongated, integral flexible catheter tube with a plurality of independent lumens that may be adapted for attachment to a separate fluid conveying device whereby fluids may be separately infused into the vein without becoming mixed, and blood may be withdrawn and venous pressure monitored simultaneously with fluid infusion. See, e.g., U.S. Pat. No. 4,072,146. The CVC may be a multi-lumen catheter composed of a central flexible lumen with a formed fluid passageway and a plurality of collapsible lumens mounted around the periphery of the central lumen also having formed fluid passageways therein. See, e.g., U.S. Pat. No. 4,406,656.

In another aspect, the CVC may have a means for preventing infection as a result of long-term use. For example, the CVC may be composed of polyurethane with a thin hydrophilic layer on the surface loaded with an antibiotic of the ramoplanin group to inhibit bacterial colonization on the catheter after insertion. See, e.g., U.S. Pat. No. 5,752,941. The CVC may be composed of a polymeric material that has an outer surface embedded by atoms of an antimicrobial metal (e.g., silver) that extend in a subsurface stratum to form a nonleaching surface treatment. See, e.g., U.S. Pat. No. 5,520,664.

In another aspect, the CVC may be used with an apparatus that provides a means of controlling the injection or withdrawal of bodily fluids through the CVC. For example, the CVC apparatus may be composed of a syringe body with two barrels that have two separate fluid conduits with independent plungers and a valve body. See, e.g., U.S. Pat. No. 5,411,485. The CVC apparatus may be composed of an upper and lower molded sheets and a plurality of syringe channels and barrels that are individually operated by syringe plungers. See, e.g., U.S. Pat. No. 5,417,667. The CVC apparatus may be an integrally molded base sheet which forms opposed slide valve walls that have a plurality of syringes mounted for fluid communication with the inlet ports. See, e.g., U.S. Pat. No. 5,454,792. The CVC apparatus may be composed with access apparatus to provide easier accessibility by being composed of a connector that is in bi-directional fluid communication between a manifold and a CVC. See, e.g., U.S. Pat. No. 5,308,322. The CVC apparatus may be a valve assembly that is provided for the distal end of a CVC for controlling fluid passage from the catheter to the blood flow passage in which it is inserted. See, e.g., U.S. Pat. No. 5,030,210.

Other examples of central venous catheters include total parenteral nutrition catheters, peripherally inserted central venous catheters, flow-directed balloon-tipped pulmonary artery catheters, long-term central venous access catheters (such as Hickman lines and Broviac catheters). Representative examples of such catheters are described in U.S. Pat. Nos. 3,995,623, 4,072,146 4,096,860, 4,099,528, 4,134,402, 4,180,068, 4,385,631, 4,406,656, 4,568,329, 4,960,409, 5,176,661, 5,916,208.

CVCs, which may be combined with one or more agents according to the present invention, include commercially available products. For example, Bard Access Systems (Salt Lake City, Utah) which is a division of C.R. Bard sells the HICKMAN, BROVIAC and LEONARD Central Venous Catheters which are available with SureCuff tissue in-growth cuff and the VitaCuff Antimicrobial Cuff. Edward Lifesciences (Irvine, Calif.) sells the VANTEX Catheter as well as the PRESEP CENTRAL VENOUS OXIMETRY Catheter. Cook Critical Care (Bloomington, Ind.) sells the SPECTRUM Antibiotic Impregnated Catheters as well as other CVC sets and trays. Arrow International (Reading, Pa.) sells the ARROWGARD BLUE Catheters that have single or multiple lumens.

A variety of central venous catheters are available for use in hemodialysis including, but not restricted to, catheters which are totally implanted such as the Lifesite (Vasca Inc., Tewksbury, Mass.) and the Dialock (Biolink Corp., Middleboro, Mass.). Central venous catheters are prone to infection and embodiments for that purpose are described above.

In one aspect, the present invention provides CVC devices that include an anti-scarring agent or a composition that includes an anti-scarring agent. Numerous polymeric and non-polymeric delivery systems have been described above. These compositions can further comprise one or more fibrosis-inhibiting agents such that the overgrowth of granulation tissue is inhibited or reduced.

Methods for incorporating the fibrosis-inhibiting agent into or onto the device includes: (a) directly affixing to the device a fibrosis-inhibiting composition (e.g., by either a spraying process or dipping process as described above, with or without a carrier), (b) directly incorporating into the device a fibrosis-inhibiting composition (e.g., by either a spraying process or dipping process as described above, with or without a carrier, (c) by coating the device with a substance such as a hydrogel which will in turn absorb the fibrosis-inhibiting composition, (d) by interweaving fibrosis-inhibiting composition coated thread (or the polymer itself formed into a thread) into the device structure, (e) constructing the device itself or a portion of the device with a fibrosis-inhibiting composition, or (f) by covalently binding the fibrosis-inhibiting agent directly to the device surface or to a linker (small molecule or polymer) that is coated or attached to the device surface. The coatings can be applied to different portions of the device. For example, the coating can be (a) as a coating applied to the external surface of the tube; (b) as a coating applied to the internal (luminal) surface of the tube; (c) as a coating applied to the ends of the tube; and/or (d) as a coating applied to all or parts of both surfaces of the tube.

In addition to coating the device with the fibrosis-inhibiting composition, the fibrosis-inhibiting agent can be mixed with the materials that are used to make the device such that the fibrosis-inhibiting agent is incorporated into the final device.

In addition to incorporation of a fibrosis-inhibiting agent into or onto the device, another biologically active agent can be incorporated into or onto the device, for example an anti-inflammatory (e.g., dexamethazone or aspirin), antithrombotic agents (e.g., heparin, heparin complexes, hydrophobic heparin derivatives, aspirin, or dipyridamole) and/or an antibiotic (e.g., amoxicillin, trimethoprim-sulfamethoxazole, azithromycin, clarithromycin, amoxicillin-clavulanate, cefprozil, cefuroxime, cefpodoxime, or cefdinir).

According to the present invention, any anti-scarring agent described above can be utilized in the practice of this embodiment. Within one embodiment of the invention, CVC devices may be adapted to release an agent that inhibits one or more of the four general components of the process of fibrosis (or scarring), including: formation of new blood vessels (angiogenesis), migration and proliferation of connective tissue cells (such as fibroblasts or smooth muscle cells), deposition of extracellular matrix (ECM), and remodeling (maturation and organization of the fibrous tissue). By inhibiting one or more of the components of fibrosis (or scarring), the overgrowth of granulation tissue may be inhibited or reduced.

Examples of fibrosis-inhibiting agents for use in CVC devices include the following: cell cycle inhibitors including (A) anthracyclines (e.g., doxorubicin and mitoxantrone), (B) taxanes (e.g., paclitaxel, TAXOTERE and docetaxel), and (C) podophyllotoxins (e.g., etoposide); (D) immunomodulators (e.g., sirolimus, everolimus, tacrolimus); (E) heat shock protein 90 antagonists (e.g., geldanamycin); (F) HMGCoA reductase inhibitors (e.g., simvastatin); (G) inosine monophosphate dehydrogenase inhibitors (e.g., mycophenolic acid, 1-alpha-25 dihydroxy vitamin D₃); (H)NF kappa B inhibitors (e.g., Bay 11-7082); (I) antimycotic agents (e.g., sulconizole), (J) p38 MAP kinase inhibitors (e.g., SB202190), and (K) and anti-angiogenesis agents (e.g., halofuginone bromide), as well as analogues and derivatives of the aforementioned.

As CVC devices are made in a variety of configurations and sizes, the exact dose administered will vary with device size, surface area and design. However, certain principles can be applied in the application of this art. Drug dose can be calculated as a function of dose per unit area (of the portion of the device being coated), total dose administered, and appropriate surface concentrations of active drug can be determined. Drugs are to be used at concentrations that range from several times more than to 10%, 5%, or even less than 1% of the concentration typically used in a single chemotherapeutic systemic dose application. Preferably, the drug is released in effective concentrations for a period ranging from 1-90 days.

Regardless of the method of application of the drug to the device, the exemplary anti-fibrosing agents, used alone or in combination, should be administered under the following dosing guidelines. The total amount (dose) of anti-scarring agent in or on the device may be in the range of about 0.01 μg-10 μg, or 10 μg-10 mg, or 10 mg-250 mg, or 250 mg-1000 mg, or 1000 mg-2500 mg. The dose (amount) of anti-scarring agent per unit area of device surface to which the agent is applied may be in the range of about 0.01 μg/mm²-1 μg/mm², or 1 μg/mm²-10 μg/mm², or 10 μg/mm²-250 μg/mm², 250 μg/mm²-1000 μg/mm², or 1000 μg/mm² 2500 μg/mm².

Provided below are exemplary dosage ranges for various anti-scarring agents that can be used in conjunction with CVC devices in accordance with the invention. A) Cell cycle inhibitors including doxorubicin and mitoxantrone. Doxorubicin analogues and derivatives thereof: total dose not to exceed 25 mg (range of 0.1 μg to 25 mg); preferred 1 μg to 5 mg. The dose per unit area of 0.01 μg-100 μg per mm²; preferred dose of 0.1 μg/mm²-10 μg/mm². Minimum concentration of 10⁻⁸-10⁻⁴ M of doxorubicin is to be maintained on the device surface. Mitoxantrone and analogues and derivatives thereof: total dose not to exceed 5 mg (range of 0.01 μg to 5 mg); preferred 0.1 μg to 1 mg. The dose per unit area of the device of 0.01 μg-20 μg per mm²; preferred dose of 0.05 μg/mm²-3 μg/mm². Minimum concentration of 10⁻⁸-10⁴ M of mitoxantrone is to be maintained on the device surface. B) Cell cycle inhibitors including Paclitaxel and analogues and derivatives (e.g., docetaxel) thereof: total dose not to exceed 10 mg (range of 0.1 μg to 10 mg); preferred 1 μg to 3 mg. The dose per unit area of the device of 0.1 μg-10 μg per mm²; preferred dose of 0.25 μg/mm²-5 μg/mm². Minimum concentration of 10-8104 M of paclitaxel is to be maintained on the device surface. (C) Cell cycle inhibitors such as podophyllotoxins (e.g., etoposide): total dose not to exceed 10 mg (range of 0.1 μg to 10 mg); preferred 1 μg to 3 mg. The dose per unit area of the device of 0.1 μg-10 μg per mm²; preferred dose of 0.25 μg/mm²-5 μg/mm². Minimum concentration of 10⁻⁸-10⁻⁴ M of etoposide is to be maintained on the device surface. (D) Immunomodulators including sirolimus and everolimus. Sirolimus (i.e., rapamycin, RAPAMUNE): Total dose not to exceed 10 mg (range of 0.1 μg to 10 mg); preferred 10 μg to 1 mg. The dose per unit area of 0.1 μg-100 μg per mm²; preferred dose of 0.5 μg/mm²-10 μg/mm². Minimum concentration of 10⁻⁸-10⁻⁴ M is to be maintained on the device surface. Everolimus and derivatives and analogues thereof: Total dose should not exceed 10 mg (range of 0.1 μg to 10 mg); preferred 10 μg to 1 mg. The dose per unit area of 0.1 μg-100 μg per mm² of surface area; preferred dose of 0.3 μg/mm²-10 μg/mm². Minimum concentration of 10⁻⁸-10⁻⁴ M of everolimus is to be maintained on the device surface. (E) Heat shock protein 90 antagonists (e.g., geldanamycin) and analogues and derivatives thereof: total dose not to exceed 20 mg (range of 0.1 μg to 20 mg); preferred 1 μg to 5 mg. The dose per unit area of the device of 0.1 μg-10 μg per mm²; preferred dose of 0.25 μg/mm²-5 μg/mm². Minimum concentration of 10⁻⁸-10⁻⁴ M of geldanamycin is to be maintained on the device surface. (F) HMGCoA reductase inhibitors (e.g., simvastatin) and analogues and derivatives thereof: total dose not to exceed 2000 mg (range of 10.0 μg to 2000 mg); preferred 10 μg to 300 mg. The dose per unit area of the device of 1.0 μg-1000 μg per mm²; preferred dose of 2.5 μg/mm²-500 μg/mm². Minimum concentration of 10⁻⁸-10⁻³ M of simvastatin is to be maintained on the device surface. (G) Inosine monophosphate dehydrogenase inhibitors (e.g., mycophenolic acid, 1-alpha-25 dihydroxy vitamin D₃) and analogues and derivatives thereof: total dose not to exceed 2000 mg (range of 10.0 μg to 2000 mg); preferred 10 μg to 300 mg. The dose per unit area of the device of 1.0 μg-1000 μg per mm²; preferred dose of 2.5 μg/mm²-500 μg/mm². Minimum concentration of 10⁻⁸-10⁻³ M of mycophenolic acid is to be maintained on the device surface. (H)NF kappa B inhibitors (e.g., Bay 11-7082) and analogues and derivatives thereof: total dose not to exceed 200 mg (range of 1.0 μg to 200 mg); preferred 1 μg to 50 mg. The dose per unit area of the device of 1.0 μg-100 μg per mm²; preferred dose of 2.5 μg/mm²-50 μg/mm². Minimum concentration of 10⁻⁸-10⁻⁴ M of Bay 11-7082 is to be maintained on the device surface. (I) Antimycotic agents (e.g., sulconizole) and analogues and derivatives thereof: total dose not to exceed 2000 mg (range of 10.0 μg to 2000 mg); preferred 10 μg to 300 mg. The dose per unit area of the device of 1.0 μg-1000 μg per mm²; preferred dose of 2.5 μg/mm²-500 μg/mm². Minimum concentration of 10⁻⁸-10⁻³ M of sulconizole is to be maintained on the device surface. (J) p38 MAP kinase inhibitors (e.g., SB202190) and analogues and derivatives thereof: total dose not to exceed 2000 mg (range of 10.0 μg to 2000 mg); preferred 10 μg to 300 mg. The dose per unit area of the device of 1.0 μg-1000 μg per mm²; preferred dose of 2.5 μg/mm²-500 μg/mm². Minimum concentration of 10⁻⁸-10⁻³ M of SB202190 is to be maintained on the device surface. (K) Anti-angiogenic agents (e.g., halofuginone bromide) and analogues and derivatives thereof: total dose not to exceed 10 mg (range of 0.1 μg to 10 mg); preferred 1 μg to 3 mg. The dose per unit area of the device of 0.1 μg-10 μg per mm²; preferred dose of 0.25 μg/mm²-5 μg/mm². Minimum concentration of 10⁻⁸-10⁻⁴ M of halofuginone bromide is to be maintained on the device surface.

In addition to those described above (e.g., sirolimus, everolimus, and tacrolimus), several other examples of immunomodulators and appropriate dosages ranges for use with central venous catheter devices include the following: (A) Biolimus and derivatives and analogues thereof: Total dose should not exceed 10 mg (range of 0.1 μg to 10 mg); preferred 10 μg to 1 mg. The dose per unit area of 0.1 μg-100 μg per mm² of surface area; preferred dose of 0.3 μg/mm²-10 μg/mm². Minimum concentration of 10⁻⁸-10⁻⁴ M of everolimus is to be maintained on the device surface. (B) Tresperimus and derivatives and analogues thereof: Total dose should not exceed 10 mg (range of 0.1 μg to 10 mg); preferred 10 μg to 1 mg. The dose per unit area of 0.1 μg-100 μg per mm² of surface area; preferred dose of 0.3 μg/mm²-10 μg/mm². Minimum concentration of 10⁻⁸-10⁻⁴ M of tresperimus is to be maintained on the device surface. (C) Auranofin and derivatives and analogues thereof: Total dose should not exceed 10 mg (range of 0.1 μg to 10 mg); preferred 10 μg to 1 mg. The dose per unit area of 0.1 μg-100 μg per mm² of surface area; preferred dose of 0.3 μg/mm²-10 μg/mm². Minimum concentration of 10⁻⁸-10⁻⁴ M of auranofin is to be maintained on the device surface. (D) 27-0-Demethylrapamycin and derivatives and analogues thereof: Total dose should not exceed 10 mg (range of 0.1 μg to 10 mg); preferred 10 μg to 1 mg. The dose per unit area of 0.1 μg-100 μg per mm² of surface area; preferred dose of 0.3 μg/mm²-10 μg/mm². Minimum concentration of 10⁻⁸-10⁻⁴ M of 27-0-Demethylrapamycin is to be maintained on the device surface. (E) Gusperimus and derivatives and analogues thereof: Total dose should not exceed 10 mg (range of 0.1 μg to 10 mg); preferred 10 μg to 1 mg. The dose per unit area of 0.1 μg-100 μg per mm² of surface area; preferred dose of 0.3 μg/mm²-10 μg/mm². Minimum concentration of 10⁻⁸-10⁻⁴ M of gusperimus is to be maintained on the device surface. (F) Pimecrolimus and derivatives and analogues thereof: Total dose should not exceed 10 mg (range of 0.1 μg to 10 mg); preferred 10 μg to 1 mg. The dose per unit area of 0.1 μg-100 μg per mm² of surface area; preferred dose of 0.3 μg/mm²-10 μg/mm². Minimum concentration of 10⁻⁸-10⁻⁴ M of pimecrolimus is to be maintained on the device surface and (G) ABT-578 and analogues and derivatives thereof: Total dose should not exceed 10 mg (range of 0.1 μg to 10 mg); preferred 10 μg to 1 mg. The dose per unit area of 0.1 μg-100 μg per mm² of surface area; preferred dose of 0.3 μg/mm²-10 μg/mm². Minimum concentration of 10⁻⁸-10⁻⁴ M of ABT-578 is to be maintained on the device surface.

In addition to those described above (e.g., paclitaxel, TAXOTERE, and docetaxel), several other examples of anti-microtubule agents and appropriate dosages ranges for use with central venous catheter devices include vinca alkaloids such as vinblastine and vincristine sulfate and analogues and derivatives thereof: total dose not to exceed 10 mg (range of 0.1 μg to 10 mg); preferred 1 μg to 3 mg. Dose per unit area of the device of 0.1 μg-10 μg per mm²; preferred dose of 0.25 μg/mm²-5 μg/mm². Minimum concentration of 10⁻⁸-10⁻⁴ M of drug is to be maintained on the device surface.

Ventricular Assist Devices

In one aspect, the present invention provides for the combination of an anti-scarring agent and a ventricular assist device (VAD).

Ventrical assist devices are intended to assist the native heart in pumping blood throughout the body. Examples of VADs and other related devices include, without limitation, left ventricular assist devices, right ventricular assist devices, biventricular assist devices, cardiac assist devices, mechanical assist devices, artificial cardiac assist devices, implantable heart assist systems, implantable ventricular assist devices, heart assist pumps and intra-ventricular cardiac assist devices.

VADs are used to treat heart failure where the heart is incapable of pumping blood throughout the body at the rate needed to maintain adequate blood flow. Heart failure includes, without limitation, acute myocardial infarction, cardiomyopathy, cardiac valvular dysfunction, extensive cardiac surgery and uncontrolled cardiac arrhythmias. VADs assist the failing heart by increasing its pumping ability and allowing the heart to rest to recover its normal pumping function. In general, VADs are typically composed of a blood pump that is attached between the ventricle and aorta, cannulae that connect the pump to the heart, and a drive console that powers and controls the device. The most common VAD that exists is the left VAD because the left ventricle of the heart becomes diseased more often than the right ventricle; however, VADs may be used to pump blood from the left ventricle, right ventricle or both ventricles. VADs may be categorized by the pumping drives, which may function as either pulsatile (e.g., intra-aortic balloon pumps) or continuous, (e.g., reciprocating piston-type pumps or rotary pumps (centrifugal or axial impellers)).

VADs, however, may have medical complications associated with the implantation or prolonged use, such as, infections, septic emboli, hemorrhaging, inflammation as a reaction to tissue damage, and thrombosis induced by coagulation or blood stasis. These complications may obstruct the utility of the VAD and may lead to life threatening events. Incorporation of an anti-scarring agent into a VAD may prevent stenosis and/or obstruction of the device.

In one aspect, the VAD may be a pulsatile pump. These devices may have flexible sacks or diaphragms which are compressed and released to provide pulsatile pumping action. One type of pulsatile pump is the intra-aortic balloon pumps (IABP) which is a pulsatile sack device that may be implemented using minimally invasive procedures and are most functional when the left ventricle is able to eject blood to maintain a systemic arterial pressure. For example, the VAD may be an IABP that is a temporary, removable support within the aortic arch that descends through the aorta which has both a depressurized and pressurized position which is maintained by a pumping and blocking balloon. See, e.g., U.S. Pat. No. 6,228,018. The VAD may be an IABP catheter and a pumping chamber having both a large and small diameter portions that are separated by a flexible diaphragm/membrane. See, e.g., U.S. Pat. No. 5,928,132. The VAD may be a pulsatile pump composed of a cannula with an outer sheath and lumen, intake and outlet valves, fluid reservoir, and hydraulic pump that produces a pulsatile pumping action of blood through the cannula. See, e.g., U.S. Pat. No. 6,007,479.

In another aspect, the VAD may be a continuous pump providing mostly steady flow of blood which may include an imperceptible pulsatile component. Continuous pumps may include reciprocating piston-type pumps, such as pneumatically powered devices or magnetically operated devices, and rotary pumps, such as centrifugal or axial impellers. For example, the VAD may be an implantable apparatus with a stator member and a magnetically suspended rotor member that act as a centrifugal pump where an impeller draws blood from the left ventricle and delivers it to the aorta thereby reducing the left ventricle pressure. See, e.g., U.S. Pat. No. 5,928,131. The VAD may be composed of an implantable reciprocating piston for driving an implanted blood-pumping mechanism which is controlled by external electromagnets. See, e.g., U.S. Pat. No. 5,089,017.

In another aspect, the VAD may be a device for assisting the pumping capacity of one of either the left or right ventricle. For example, the VAD may be composed of a housing apparatus with a pair of chambers with an inlet and outlet port, at least one ventricular outflow conduit, and an actuator that contracts one of the chambers while expanding the other to provide a positive displacement pump. See, e.g., U.S. Pat. No. 6,264,601. The VAD may be composed of a pump, a chamber above the pump, and a tube that connects the pump and chamber using liquid and gas as a means for communication. See, e.g., U.S. Pat. No. 6,146,325.

In another aspect, the VAD may be a device designed specifically for the left ventricle. For example, the VAD may be a blood pump adapted to be joined in flow communication between the left ventricle and the aorta using an inlet flow pressure sensor and a controller that may adjust speed of pump based on sensor feedback. See, e.g., U.S. Pat. No. 6,623,420. The VAD may be composed of a bag adapted to expand by being filled with blood and able to contract to expel the blood, and the means for varying the resistance of the bag by using gaseous substance through a duct to a containing casing. See, e.g., U.S. Pat. No. 6,569,079. The VAD may be a pump system composed of a deformable sac with inlet and outlet means and a pair of plates on opposite sides of the sac to deform the sac. See, e.g., U.S. Pat. No. 5,599,173.

In another aspect, the VAD may be a device designed as a biventricular assist device. For example, the VAD may be a biventricular assist device composed of a self-supporting cup having an annular diaphragm that forms a fluid chamber around the heart cavity whereby it may have a pressure inlet/port that communicates with the fluid chamber to regulate positive and negative pressures. See, e.g., U.S. Pat. Nos. 5,908,378; 5,749,839 and 5,738,627.

In another aspect, the VAD may be an implanted system used to supplement the pumping of blood circulation from a location outside the heart. For example, the VAD may be an extracardiac pumping system composed of an inflow and outflow conduit fluidly coupled to the pump (e.g., pulsatile or rotary pump) and a control circuit to synchronously actuate the pump. See, e.g., U.S. Pat. Nos. 6,610,004; 6,428,464 and 6,200,260.

In another aspect, the VAD related devices may be a used in conjunction with VADs or as stand alone to treat congestive heart failure victims. For example, a VAD related device may be a reinforcement device composed of a jacket that is applied to the heart to constrain cardiac expansion to a predetermined limit. See, e.g., U.S. Pat. Nos. 6,582,355; 6,567,699; 6,241,654 and 6,169,922.

Representative examples of VADs, which may be combined with one or more agents according to the present invention, include commercially available products. For example, Thoratec Corporation (Pleasanton, Calif.) sells the HEARTMATE Left Ventricular Assist Systems. WorldHeart Corporation (ON, Canada) sells the WORLDHEART NOVACOR Left Ventricular Assist System. Arrow International (Reading, Pa.) sells the LIONHEART Left Ventricular Assist System.

In one aspect, the present invention provides LVAD devices that include an anti-scarring agent or a composition that includes an anti-scarring agent. Numerous polymeric and non-polymeric delivery systems have been described above. These compositions can further comprise one or more fibrosis-inhibiting agents such that the overgrowth of granulation tissue is inhibited or reduced.

Methods for incorporating the fibrosis-inhibiting agent into or onto the device includes: (a) directly affixing to the device a fibrosis-inhibiting composition (e.g., by either a spraying process or dipping process as described above, with or without a carrier), (b) directly incorporating into the device a fibrosis-inhibiting composition (e.g., by either a spraying process or dipping process as described above, with or without a carrier, (c) by coating the device with a substance such as a hydrogel which will in turn absorb the fibrosis-inhibiting composition, (d) by interweaving fibrosis-inhibiting composition coated thread (or the polymer itself formed into a thread) into the device structure, (e) constructing the device itself or a portion of the device with a fibrosis-inhibiting composition, or (f) by covalently binding the fibrosis-inhibiting agent directly to the device surface or to a linker (small molecule or polymer) that is coated or attached to the device surface. The coatings can be applied to different portions of the device. For example, the coating can be (a) as a coating applied to the external surface of the tube that leads out of the left ventricle; (b) as a coating applied to the internal (luminal) surface of the tube that leads out of the left ventricle; (c) as a coating applied to external surface of the tube that lead to the aorta; (d) as a coating applied to internal (luminal) surface of the tube that lead to the aorta; (e) as a coating that is applied to the ends of the tube where they are in contact with the heart tissue, and/or (f) as a coating applied to all or parts of the entire device.

In addition to coating the device with the fibrosis-inhibiting composition, the fibrosis-inhibiting agent can be mixed with the materials that are used to make the device such that the fibrosis-inhibiting agent is incorporated into the final device.

In addition to incorporation of a fibrosis-inhibiting agent into or onto the device, another biologically active agent can be incorporated into or onto the device, for example an anti-inflammatory (e.g., dexamethazone or aspirin), antithrombotic agents (e.g., heparin, heparin complexes, hydrophobic heparin derivatives, aspirin, or dipyridamole) and/or an antibiotic (e.g., amoxicillin, trimethoprim-sulfamethoxazole, azithromycin, clarithromycin, amoxicillin-clavulanate, cefprozil, cefuroxime, cefpodoxime, or cefdinir).

According to the present invention, any anti-scarring agent described above can be utilized in the practice of this embodiment. Within one embodiment of the invention, VAD devices (e.g., LVAD's) may be adapted to release an agent that inhibits one or more of the four general components of the process of fibrosis (or scarring), including: formation of new blood vessels (angiogenesis), migration and proliferation of connective tissue cells (such as fibroblasts or smooth muscle cells), deposition of extracellular matrix (ECM), and remodeling (maturation and organization of the fibrous tissue). By inhibiting one or more of the components of fibrosis (or scarring), the overgrowth of granulation tissue may be inhibited or reduced.

Examples of fibrosis-inhibiting agents for use in left ventricular assist devices include the following: cell cycle inhibitors such as (A) anthracyclines (e.g., doxorubicin and mitoxantrone), (B) taxanes (e.g., paclitaxel, TAXOTERE and docetaxel), and (C) podophyllotoxins (e.g., etoposide); (D) immunomodulators (e.g., sirolimus, everolimus, tacrolimus); (E) heat shock protein 90 antagonists (e.g., geldanamycin); (F) HMGCoA reductase inhibitors (e.g., simvastatin); (G) inosine monophosphate dehydrogenase inhibitors (e.g., mycophenolic acid, 1-alpha-25 dihydroxy vitamin D₃); (H)NF kappa B inhibitors (e.g., Bay 11-7082); (I) antimycotic agents (e.g., sulconizole), (J) p38 MAP kinase inhibitors (e.g., SB202190), and (K) and anti-angiogenesis agents (e.g., halofuginone bromide), as well as analogues and derivatives of the aforementioned.

As ventricular assist devices are made in a variety of configurations and sizes, the exact dose administered will vary with device size, surface area and design. However, certain principles can be applied in the application of this art. Drug dose can be calculated as a function of dose per unit area (of the portion of the device being coated), total dose administered, and appropriate surface concentrations of active drug can be determined. Drugs are to be used at concentrations that range from several times more than to 10%, 5%, or even less than 1% of the concentration typically used in a single chemotherapeutic systemic dose application. Preferably, the drug is released in effective concentrations for a period ranging from 1-90 days.

Regardless of the method of application of the drug to the device, the exemplary anti-fibrosing agents, used alone or in combination, should be administered under the following dosing guidelines. The total amount (dose) of anti-scarring agent in or on the device may be in the range of about 0.01 μg-10 μg, or 10 μg-10 mg, or 10 mg-250 mg, or 250 mg-1000 mg, or 1000 mg-2500 mg. The dose (amount) of anti-scarring agent per unit area of device surface to which the agent is applied may be in the range of about 0.01 μg/mm²-1 μg/mm², or 1 μg/mm²-10 μg/mm², or 10 μg/mm²-250 μg/mm², 250 μg/mm²-1000 μg/mm², or 1000 μg/mm²-2500 μg/mm².

Provided below are exemplary dosage ranges for various anti-scarring agents that can be used in conjunction with ventricular assist devices in accordance with the invention. A) Cell cycle inhibitors including doxorubicin and mitoxantrone. Doxorubicin analogues and derivatives thereof: total dose not to exceed 25 mg (range of 0.1 μg to 25 mg); preferred 1 μg to 5 mg. The dose per unit area of 0.01 μg-100 μg per mm²; preferred dose of 0.1 μg/mm²-10 μg/mm². Minimum concentration of 10⁻⁸-10⁻⁴ M of doxorubicin is to be maintained on the device surface. Mitoxantrone and analogues and derivatives thereof: total dose not to exceed 5 mg (range of 0.01 μg to 5 mg); preferred 0.1 μg to 1 mg. The dose per unit area of the device of 0.01 μg-20 μg per mm²; preferred dose of 0.05 μg/mm²-3 μg/mm². Minimum concentration of 10⁻⁸-10⁴ M of mitoxantrone is to be maintained on the device surface. B) Cell cycle inhibitors including paclitaxel and analogues and derivatives (e.g., docetaxel) thereof: total dose not to exceed 10 mg (range of 0.1 μg to 10 mg); preferred 1 μg to 3 mg. The dose per unit area of the device of 0.1 μg-10 μg per mm²; preferred dose of 0.25 μg/mm²-5 μg/mm². Minimum concentration of 10⁻⁸-10⁻⁴ M of paclitaxel is to be maintained on the device surface. (C) Cell cycle inhibitors such as podophyllotoxins (e.g., etoposide): total dose not to exceed 10 mg (range of 0.1 μg to 10 mg); preferred 1 μg to 3 mg. The dose per unit area of the device of 0.1 μg-10 μg per mm²; preferred dose of 0.25 μg/mm²-5 μg/mm². Minimum concentration of 10⁻⁸-10⁻⁴ M of etoposide is to be maintained on the device surface. (D) Immunomodulators including sirolimus and everolimus. Sirolimus (i.e., rapamycin, RAPAMUNE): Total dose not to exceed 10 mg (range of 0.1 μg to 10 mg); preferred 10 μg to 1 mg. The dose per unit area of 0.1 μg-100 μg per mm²; preferred dose of 0.5 μg/mm²-10 μg/mm². Minimum concentration of 10⁻⁸-10⁻⁴ M is to be maintained on the device surface. Everolimus and derivatives and analogues thereof: Total dose should not exceed 10 mg (range of 0.1 μg to 10 mg); preferred 10 μg to 1 mg. The dose per unit area of 0.1 μg-100 μg per mm² of surface area; preferred dose of 0.3 μg/mm²-10 μg/mm². Minimum concentration of 10⁻⁸-10⁻⁴ M of everolimus is to be maintained on the device surface. (E) Heat shock protein 90 antagonists (e.g., geldanamycin) and analogues and derivatives thereof: total dose not to exceed 20 mg (range of 0.1 μg to 20 mg); preferred 1 μg to 5 mg. The dose per unit area of the device of 0.1 μg-10 μg per mm²; preferred dose of 0.25 μg/mm²-5 μg/mm². Minimum concentration of 10⁻⁸-10⁻⁴ M of geldanamycin is to be maintained on the device surface. (F) HMGCoA reductase inhibitors (e.g., simvastatin) and analogues and derivatives thereof: total dose not to exceed 2000 mg (range of 10.0 μg to 2000 mg); preferred 10 μg to 300 mg. The dose per unit area of the device of 1.0 μg-1000 μg per mm²; preferred dose of 2.5 μg/mm²-500 μg/mm². Minimum concentration of 10⁻⁸-10⁻³ M of simvastatin is to be maintained on the device surface. (G) Inosine monophosphate dehydrogenase inhibitors (e.g., mycophenolic acid, 1-alpha-25 dihydroxy vitamin D₃) and analogues and derivatives thereof: total dose not to exceed 2000 mg (range of 10.0 μg to 2000 mg); preferred 10 μg to 300 mg. The dose per unit area of the device of 1.0 μg-1000 μg per mm²; preferred dose of 2.5 μg/mm²-500 μg/mm². Minimum concentration of 10⁻⁸-10⁻³ M of mycophenolic acid is to be maintained on the device surface. (H) NF kappa B inhibitors (e.g., Bay 11-7082) and analogues and derivatives thereof: total dose not to exceed 200 mg (range of 1.0 μg to 200 mg); preferred 1 μg to 50 mg. The dose per unit area of the device of 1.0 μg-100 μg per mm²; preferred dose of 2.5 μg/mm²-50 μg/mm². Minimum concentration of 10⁻⁸-10⁻⁴ M of Bay 11-7082 is to be maintained on the device surface. (I) Antimycotic agents (e.g., sulconizole) and analogues and derivatives thereof: total dose not to exceed 2000 mg (range of 10.0 μg to 2000 mg); preferred 10 μg to 300 mg. The dose per unit area of the device of 1.0 μg-1000 μg per mm²; preferred dose of 2.5 μg/mm²-500 μg/mm². Minimum concentration of 10⁻⁸-10⁻³ M of sulconizole is to be maintained on the device surface. (J) p38 MAP kinase inhibitors (e.g., SB202190) and analogues and derivatives thereof: total dose not to exceed 2000 mg (range of 10.0 μg to 2000 mg); preferred 10 μg to 300 mg. The dose per unit area of the device of 1.0 μg-1000 μg per mm²; preferred dose of 2.5 μg/mm²-500 μg/mm². Minimum concentration of 10⁻⁸-10⁻³ M of SB202190 is to be maintained on the device surface. (K) Anti-angiogenic agents (e.g., halofuginone bromide) and analogues and derivatives thereof: total dose not to exceed 10 mg (range of 0.1 μg to 10 mg); preferred 1 μg to 3 mg. The dose per unit area of the device of 0.1 μg-10 μg per mm²; preferred dose of 0.25 μg/mm²-5 μg/mm². Minimum concentration of 10⁻⁸-10⁻⁴ M of halofuginone bromide is to be maintained on the device surface.

In addition to those described above (e.g., sirolimus, everolimus, and tacrolimus), several other examples of immunomodulators and appropriate dosages ranges for use with ventricular assist devices include the following: (A) Biolimus and derivatives and analogues thereof: Total dose should not exceed 10 mg (range of 0.1 μg to 10 mg); preferred 10 μg to 1 mg. The dose per unit area of 0.1 μg-100 μg per mm² of surface area; preferred dose of 0.3 μg/mm²-10 μg/mm². Minimum concentration of 10⁻⁸-10⁻⁴ M of everolimus is to be maintained on the device surface. (B) Tresperimus and derivatives and analogues thereof: Total dose should not exceed 10 mg (range of 0.1 μg to 10 mg); preferred 10 μg to 1 mg. The dose per unit area of 0.1 μg-100 μg per mm² of surface area; preferred dose of 0.3 μg/mm²-10 μg/mm². Minimum concentration of 10⁻⁸-10⁻⁴ M of tresperimus is to be maintained on the device surface. (C) Auranofin and derivatives and analogues thereof: Total dose should not exceed 10 mg (range of 0.1 μg to 10 mg); preferred 10 μg to 1 mg. The dose per unit area of 0.1 μg-100 μg per mm² of surface area; preferred dose of 0.3 μg/mm²-10 μg/mm². Minimum concentration of 10⁻⁸-10⁻⁴ M of auranofin is to be maintained on the device surface. (D) 27-0-Demethylrapamycin and derivatives and analogues thereof: Total dose should not exceed 10 mg (range of 0.1 μg to 10 mg); preferred 10 μg to 1 mg. The dose per unit area of 0.1 μg-100 μg per mm² of surface area; preferred dose of 0.3 μg/mm²-10 μg/mm². Minimum concentration of 10⁻⁸-10⁻⁴ M of 27-0-Demethylrapamycin is to be maintained on the device surface. (E) Gusperimus and derivatives and analogues thereof: Total dose should not exceed 10 mg (range of 0.1 μg to 10 mg); preferred 10 μg to 1 mg. The dose per unit area of 0.1 μg-100 μg per mm² of surface area; preferred dose of 0.3 μg/mm²-10 μg/mm². Minimum concentration of 10⁻⁸-10⁻⁴ M of gusperimus is to be maintained on the device surface. (F) Pimecrolimus and derivatives and analogues thereof: Total dose should not exceed 10 mg (range of 0.1 μg to 10 mg); preferred 10 μg to 1 mg. The dose per unit area of 0.1 μg-100 μg per mm² of surface area; preferred dose of 0.3 μg/mm²-10 μg/mm². Minimum concentration of 10⁻⁸-10⁻⁴ M of pimecrolimus is to be maintained on the device surface and (G) ABT-578 and analogues and derivatives thereof: Total dose should not exceed 10 mg (range of 0.1 μg to 10 mg); preferred 10 μg to 1 mg. The dose per unit area of 0.1 μg-100 μg per mm² of surface area; preferred dose of 0.3 μg/mm²-10 μg/mm². Minimum concentration of 10⁻⁸-10⁻⁴ M of ABT-578 is to be maintained on the device surface.

In addition to those described above (e.g., paclitaxel, TAXOTERE, and docetaxel), several other examples of anti-microtubule agents and appropriate dosages ranges for use with ventricular assist devices include vinca alkaloids such as vinblastine and vincristine sulfate and analogues and derivatives thereof: total dose not to exceed 10 mg (range of 0.1 μg to 10 mg); preferred 1 μg to 3 mg. Dose per unit area of the device of 0.1 μg-10 μg per mm²; preferred dose of 0.25 μg/mm²-5 μg/mm². Minimum concentration of 10⁻⁸-10⁻⁴ M of drug is to be maintained on the device surface.

Spinal Implants

In one aspect, the present invention provides for the combination of an anti-scarring agent and a spinal implant (e.g., a spinal prosthesis). As used herein, the term “spinal prostheses” refers to devices that are located in, on, or near the spine and which enhance the ability of the spine to perform its function in the host. Spinal prostheses may be used to treat the vertebral column following degeneration or damage to the spine or a component or portion thereof. In healthy hosts, the vertebral column is composed of vertebral bone plates separated by intervertebral discs that form strong joints and absorb spinal compression. The intervertebral disc is comprised of an inner gel-like substance called the nucleus pulposus with surrounding tough fibrocartilagenous fibers called the annulus fibrosis. When damage occurs to the intervertebral disc, the host can develop spinal dysfunction, crippling pain, as well as long-term disability. Typically, damage to an intervertebral disc requires surgery which often results in the fusion of adjacent vertebral bone plates using various techniques and devices. Fusion of vertebral segments alleviates the pain by restricting vertebral motion at the damaged intervertebral disc. When only one vertebral segment is fused, the host will not have any noticeable motion limitations. However, when two or more segments are fused, the normal motion of the back may become limited and thus, pain relief may not resolve due to the additional stress that is induced across the remaining vertebral joints.

In one aspect, the damaged vertebral segment may be treated using a spinal prosthesis that induces fusion between the vertebral plates. This may be conducted when only one vertebral segment is damaged. In another aspect, the damaged vertebral segment may be treated using a spinal prosthesis that maintains vertebral movement within the vertebral joint. This may be conducted when damage to more than one vertebral segment occurs.

Examples of spinal prostheses include, without limitation, spinal discs and related devices including vertebral implants, vertebral disc prostheses, lumbar disc implants, cervical disc implants, intervertebral discs, implantable prostheses, spinal prostheses, artificial discs, prosthetic implants, prosthetic spinal discs, spinal disc endoprostheses, spinal implants, artificial spinal discs, intervertebral implants, implantable spinal grafts, implantable bone grafts, artificial lumbar discs, spinal nucleus implants, and intervertebral disc spacers. Also included within the term spinal prostheses are fusion cages and related devices including fusion baskets, fusion cage apparatus, interbody cages, interbody implants, fusion devices, fusion cage anchoring devices, bone fixation apparatus, bone fixation instrumentation, bone fixation devices, fusion stabilization chamber, fusion cage anchoring plates, anchoring bone plates and bone screws.

A spinal prosthesis according to the present invention may be composed of a single material or a variety of materials including, without limitation, allograft bone material (see, e.g., U.S. Pat. No. 6,143,033), metals (see, e.g., U.S. Pat. No. 4,955,908), and/or synthetic materials (see, e.g., U.S. Pat. Nos. 6,264,695, 6,419,706, 5,824,093 and 4,911,718). The prosthesis must be biocompatible. It may consist of biodegradable or non-biodegradable components depending on the intended function of the device. See, e.g., U.S. Pat. No. 4,772,287. The spinal prosthesis may be biologically inert and serve as a mechanical means of stabilizing the vertebral column (see, e.g., U.S. Pat. Nos. 4,955,908 and 5,716,415) or it may be biologically active and serve to promote fusion with the adjacent vertebral bone plates (see, e.g., U.S. Pat. Nos. 5,489,308 and 6,520,993).

In one aspect, the prosthesis may be a fusion cage designed to promote vertebral fusion in order to limit movement between adjacent vertebrae. Fusion cages may be interbody devices that fit within the intervertebral space or they may encompass both the intervertebral space and the anterior region of the vertebral column. Fusion cages may have various shapes. For example, fusion cages may be have a rectangular shape or may be cylindrical in shape and may have a plurality of openings and helical threading. Fusion cages may have an outer body and a hollow cavity that may or may not be used to insert bone growth-promoting material for stimulating bone fusion. For example, the prosthesis may be an interbody fusion cage that has an externally threaded stem projecting from a domed outer end which is fixed using an assembly of a plate, a fastener and bone screws. See, e.g., U.S. Pat. No. 6,156,037. The prosthesis may be a fusion cage with a threaded outer surface adapted for promoting fusion with bone structures when a bone-growth-inducing substance is packed into the cage body. See, e.g., U.S. Pat. Nos. 4,961,740, 5,015,247, 4,878,915 and 4,501,269. The prosthesis may be a generally tubular shell with a helical thread projecting with a plurality of pillars with holes to facilitate bone ingrowth and mechanical anchoring. See, e.g., U.S. Pat. Nos. 6,071,310 and 5,489,308. Other U.S. patents that describe the threaded spinal implant include U.S. Pat. Nos. 5,263,953, 5,458,638 and 5,026,373.

In another aspect, the prosthesis may be a bone fixation device designed to promote vertebral fusion in order to limit movement between adjacent vertebrae. For example, bone dowels, rods, hooks, wires, wedges, plates, screws and other components may be used to fix the vertebral segments into place. The fixation device may fit within the intervertebral space or it may encompass both the intervertebral space and the anterior region of the vertebral column or it may only encompass the anterior region of the vertebral column. A bone fixation device may be used with a fusion cage to assist in stabilizing the device within the intervertebral area. For example, the prosthesis may be in the form of a solid annular body having a plurality of discrete bone-engaging teeth protruding on the superior and inferior surfaces and having a central opening that may be filled with a bone growth-promoting material. See, e.g., U.S. Pat. No. 6,520,993. The prosthesis may have a disk-like body with weld-like raised parts disposed on opposite surfaces to enhance lateral stability in situ. See, e.g., U.S. Pat. No. 4,917,704. The prosthesis may be composed of opposite end pieces that maintain the height of the intervertebral space with an integral central element that is smaller in diameter wherein osteogenic material is disposed within the annular pocket between the end pieces. See, e.g., U.S. Pat. No. 6,146,420. The prosthesis may be composed of first and second side surfaces extending parallel to each other with upper and lower surfaces that engage the adjacent vertebrae. See, e.g., U.S. Pat. No. 5,716,415. The prosthesis may be a fusion stabilization chamber composed of a hollow intervertebral spacer and an end portion with at least one hole for affixing into the surrounding bone. See, e.g., U.S. Pat. No. 6,066,175. The prosthesis may be composed of a metallic body tapering conically from the ventral to the dorsal end and having a plurality of fishplates extending from opposite sides with openings for bone screws. See, e.g., U.S. Pat. No. 4,955,908. The prosthesis may be composed of a pair of plates which may have protrusions for engaging the adjacent vertebrae and an alignment device disposed between the engaging plates for separating the plates to maintain them in lordotic alignment. See, e.g., U.S. Pat. No. 6,576,016. The prosthesis may be a plurality of implants that are inserted side by side into the disc space that promote bone fusion across an intervertebral space. See, e.g., U.S. Pat. No. 5,522,899. The prosthesis may be an anchoring device composed of an anchoring plate with a central portion configured for-attachment to a vertebral implant (e.g., fusion cage) and the end portions adapted to fasten in a fixed manner to a bony segment of the vertebra. See, e.g., U.S. Pat. No. 6,306,170. The prosthesis may be a bone fixation apparatus composed of a bone plate and a fastener apparatus (e.g., bone screws). See, e.g., U.S. Pat. Nos. 6,342,055, 6,454,769, 6,602,257 and 6,620,163.

In another aspect, the prosthesis may be an alternative to spinal fusion. The prosthesis may be a disc designed to provide normal movement between vertebral bone plates. The disc may be intended to mimic the natural shock absorbent function of the natural disc. The disc may be composed of a center core and end elements that support the disc against the adjacent vertebra or it may be intended to replace only a portion of the natural intervertebral disc (e.g., nucleus pulposus). For example, the disc may be in the form of an elastomeric section sandwiched between two rigid plates. See, e.g., U.S. Pat. Nos. 6,162,252; 5,534,030, 5,017,437 and 5,031,437. The disc may be an elongated prosthetic disc nucleus composed of a hydrogel core and a constraining flexible jacket that allows the core to deform and reform. See, e.g., U.S. Pat. No. 5,824,093. The disc may be composed of a rigid superior and inferior concaval-convex elements and a nuclear body which is located between the concave surfaces to permit movement. See, e.g., U.S. Pat. No. 6,156,067. The disc may be a partial spinal prosthesis composed of a core made of an elastic material such as silicone polymer or an elastomer which is covered by a casing made of a rigid material which is in contact with the adjacent vertebrae. See, e.g., U.S. Pat. No. 6,419,706. The disc may replace only the nucleus pulposus tissue by using a spinal nucleus implant comprised of a swellable, biomimetic plastic with a hydrophobic and hydrophilic phase which can be expanded in situ to conform to the natural size and shape. See, e.g., U.S. Pat. No. 6,264,695. The disc may be composed of a central core formed from a biocompatible elastomer wrapped by multi-layered laminae made from elastomer and fibers. See, e.g. U.S. Pat. No. 4,911,718. The disc may be composed of a fluid-filled inner bladder with an outer layer of strong, inert fibers intermingled with a bioresorbable material which promotes tissue ingrowth. See, e.g., U.S. Pat. No. 4,772,287.

In another aspect, the spinal implant may be a device that reduces spine compression or reduces adhesions that may form as a result to spinal surgery and/or trauma. For example, the device may be a protection device composed of a shield to fit onto at least one lamina on the posterior surface to prevent postoperative formation of adhesions to the spinal dura. See, e.g., U.S. Pat. Nos. 5,437,672 and 5,868,745 and U.S. Patent Application No. 2003/0078588. The device may be a prosthesis having a patch flange and a suture flange extending circumferentially around the patch such that the tissue underlying the patch is shielded and effectively nonadhesive to scar growth. See, e.g., U.S. Pat. No. 5,634,944. The device may be a protective intervening barrier composed of a biocompatible shield which is used following intraspinal or vertebral surgery to prevent postoperative adhesions from binding onto the spinal nerves. See, e.g., U.S. Pat. No. 4,013,078. The device may be used for neuro decompression while reducing fibroplasia proximate to the nerve tissue by having a surface topography texturized with outwardly-extending microstructures. See, e.g., U.S. Pat. No. 6,106,558 and U.S. Patent Application No. 2003/0078673.

Spinal prostheses and other spinal implants, which may be combined with one or more drugs according to the present invention, include commercially available products. Medtronic Sofamor Danek (Memphis, Term.) sells the fusion cage product INTERFIX Threaded Fusion Device. Centerpulse Spine-Tech (Minneapolis, Minn.) sells the BAK/C Cervical Interbody Fusion System fusion cage product and the CERVI-LOK Cervical Fixation System fixation device. Spinal Concepts (Austin, Tex.) sells the SC-ACUFIX Anterior Cervical Plate System. DePuy Spine, Inc. (Raynham, Mass.) sells the spinal discs, ACROFLEX TDR prostheses and the CHARITE Artificial Disc. Synthes-Stratec (Switzerland) sells the PRODISC system, including the PRODISC Cervical-C IDE disc replacement. Raymedica, Inc. (Minneapolis, Minn.) sells the PDN (PROSTHETIC DISC NUCLEUS).

Numerous polymeric and non-polymeric carrier systems that can be used in conjunction with spinal implants have been described above. Incorporation of a fibrosis-inhibiting agent into or onto a spinal implant can minimize fibrosis (or scarring) in the vicinity of the implant and may reduce or prevent the formation of adhesions between the implant and the surrounding tissue.

In one aspect, the present invention provides spinal implants that include an anti-scarring agent or a composition that includes an anti-scarring agent to inhibit scarring and adhesion between the device and the surrounding bone.

Methods for incorporating the anti-fibrosing compositions onto or into a spinal implant include: (a) directly affixing to the device an anti-fibrosing composition (e.g., by either a spraying process or dipping process as described above, with or without a carrier, (b) directly incorporating into the device an anti-fibrosing composition (e.g., by either a spraying process or dipping process as described above, with or without a carrier, (c) by coating the device with a substance such as a hydrogel which will in turn absorb the anti-fibrosing composition, (d) by interweaving anti-fibrosing composition coated thread (or the polymer itself formed into a thread) into the device structure, (e) by binding film or mesh which is comprised of or coated with an anti-fibrosing composition to the spinal prosthesis, (f) constructing the device itself or a portion of the device with an anti-fibrosing composition, or (g) by covalently binding the anti-fibrosing agent directly to the device surface or to a linker (small molecule or polymer) that is coated or attached to the device surface. For these devices, the coating process can be performed in such a manner as to a) coat the exterior surfaces of the device, b) coat the interior surfaces of the device or c) coat all or parts of both external and internal surface of the device.

In one aspect, a spinal implant (e.g., an implantable cages or disc) is coated with an anti-scarring agent or a composition that includes the anti-scarring agent. In certain aspects, the spinal implant may be coated with (or adapted to contain) an anti-scarring agent on one part of the device and a fibrosis-inducing agent (e.g., silk or talc) on another part of the device. For example, the outer surface of the implant (e.g., a vertebral implant) may be coated with a fibrosis-inducing agent to improve adhesion between the device and the surrounding tissue, while the interior of the device may be coated with an anti-scarring agent to minimize adhesion of tissue to the interior of the implant. Examples of fibrosis-inducing agents and methods of using fibrosis-inducing agents in combination with spinal implants are described in co-pending application entitled, “Medical Implants and Fibrosis-Inducing Agents,” filed Nov. 20, 2003 (U.S. Ser. No. 60/524,023) and Jun. 9, 2004 (U.S. Ser. No. 60/578,471).

In addition to coating the device with the anti-fibrosing composition, the anti-fibrosing agent can be mixed with the materials that are used to make the device such that the anti-fibrosing agent is incorporated into the final device.

In addition to applying the fibrosis agent to the spinal implant, an in situ forming composition, gel or thermogel composition that further comprises a fibrosis-inhibiting agent can be applied to the placement site of the spinal prosthesis, (a) prior to placement of the prosthesis, (b) after placement of the prosthesis and/or (c) both prior and post placement on the prosthesis.

For the in situ forming, thermogel and gel compositions, the fibrosis-inhibiting agents can be incorporated directly into the formulation to produced a suspension or a solution or it can be incorporated into a secondary carrier (e.g., micelles, liposomes, microspheres, microparticles, nanospheres, microparticulates, emulsions and/or microemulations) that is then incorporated into the in situ forming compositions. In another embodiment, the fibrosis-inhibiting agent can be electrostatically or covalently bound to one or more of the polymeric components of the in situ forming composition.

In another embodiment, the fibrosis-inhibiting agent can be incorporated into a biodegradable or dissolvable film or mesh that is then applied to the treatment site prior or post implantation of the prosthesis/implant. Preferred materials for the manufacture of these films or meshes are hyaluronic acid (crosslinked or non-crosslinked), cellulose derivatives (e.g., hydroxypropyl cellulose), PLGA, POLYACTIVE, collagen and crosslinked poly(ethylene glycol).

In another embodiment, a solution or suspension that further comprises a fibrosis-inhibiting agent can be applied to the placement site of the spinal prosthesis, (a) prior to placement of the prosthesis, (b) after placement of the prosthesis and/or (c) both prior and post placement on the prosthesis. The fibrosis-inhibiting agents can be incorporated directly into the formulation to produced a suspension or a solution or it can be incorporated into a secondary carrier (e.g., micelles, liposomes, microspheres, microparticles, nanospheres, microparticulates, emulsions and/or microemulations) that is then incorporated into the in situ forming compositions. This solution or suspension can be applied (sprayed, rubbed, dripped etc) onto the treatment are prior to or post prosthesis placement.

In addition to incorporation of a fibrosis-inhibiting agent into or onto the device, another biologically active agent can be incorporated into or onto the device, for example an anti-inflammatory (e.g., dexamethazone or aspirin), antithrombotic agent (e.g., heparin, heparin complexes, hydrophobic heparin derivatives, aspirin, or dipyridamole) and/or an antibiotic (e.g., amoxicillin, trimethoprim-sulfamethoxazole, azithromycin, clarithromycin, amoxicillin-clavulanate, cefprozil, cefuroxime, cefpodoxime, or cefdinir).

According to the present invention, any adhesion or fibrosis-inducing agent described above can be utilized in the practice of this embodiment. Within one embodiment of the invention, spinal implants may be adapted to release an agent that inhibits one or more of the four general components of the process of fibrosis (or scarring), including: formation of new blood vessels (angiogenesis), migration and proliferation of connective tissue cells (such as fibroblasts or smooth muscle cells), deposition of extracellular matrix (ECM), and remodeling (maturation and organization of the fibrous tissue). By inhibiting one or more of the components of fibrosis (or scarring), the overgrowth of granulation tissue may be inhibited or reduced.

Examples of fibrosis-inhibiting agents for use in spinal implants include the following: cell cycle inhibitors including (A) anthracyclines (e.g., doxorubicin and mitoxantrone), (B) taxanes (e.g., paclitaxel, TAXOTERE and docetaxel), and (C) podophyllotoxins (e.g., etoposide); (D) immunomodulators (e.g., sirolimus, everolimus, tacrolimus), (E) heat shock protein 90 antagonists (.e.g., geldanamycin); (F) HMGCoA reductase inhibitors (.e.g., simvastatin); (G) inosine monophosphate dehydrogenase inhibitors (e.g., mycophenolic acid, 1-alpha-25 dihydroxy vitamin D₃), (H)NF kappa B inhibitors (e.g., Bay 11-7082), (I) antimycotic agents (e.g., sulconizole) and (J) p38 MAP kinase inhibitors (e.g., SB202190), as well as analogues and derivatives of the aforementioned.

As spinal implants are made in a variety of configurations and sizes, the exact dose administered will vary with device size, surface area and design. However, certain principles can be applied in the application of this art. Drug dose can be calculated as a function of dose per unit area (of the portion of the device being coated), total dose administered, and appropriate surface concentrations of active drug can be determined. Drugs are to be used at concentrations that range from several times more than to 10%, 5%, or even less than 1% of the concentration typically used in a single chemotherapeutic systemic dose application. Preferably, the drug is released in effective concentrations for a period ranging from 1-90 days.

Regardless of the method of application of the drug to the device, the exemplary anti-fibrosing agents, used alone or in combination, should be administered under the following dosing guidelines. The total amount (dose) of anti-scarring agent in or on the device may be in the range of about 0.01 μg-10 μg, or 10 μg-10 mg, or 10 mg-250 mg, or 250 mg-1000 mg, or 1000 mg-2500 mg. The dose (amount) of anti-scarring agent per unit area of device surface to which the agent is applied may be in the range of about 0.01 μg/mm²-1 μg/mm², or 1 μg/mm²-10 μg/mm², or 10 μg/mm²-250 μg/mm², 250 μg/mm²-1000 μg/mm², or 1000 μg/mm²-2500 μg/mm².

Provided below are exemplary dosage ranges for various anti-scarring agents that can be used in conjunction with spinal implants and devices in accordance with the invention. A) Cell cycle inhibitors including doxorubicin and mitoxantrone. Doxorubicin analogues and derivatives thereof: total dose not to exceed 25 mg (range of 0.1 μg to 25 mg); preferred 1 μg to 5 mg. The dose per unit area of 0.01 μg-100 μg per mm²; preferred dose of 0.1 μg/mm²-10 μg/mm². Minimum concentration of 10⁻⁸-10⁻⁴ M of doxorubicin is to be maintained on the device surface. Mitoxantrone and analogues and derivatives thereof: total dose not to exceed 5 mg (range of 0.01 μg to 5 mg); preferred 0.1 μg to 1 mg. The dose per unit area of the device of 0.01 μg-20 μg per mm²; preferred dose of 0.05 μg/mm²-3 μg/mm². Minimum concentration of 10⁻⁸-10⁴ M of mitoxantrone is to be maintained on the device surface. B) Cell cycle inhibitors including Paclitaxel and analogues and derivatives (e.g., docetaxel) thereof: total dose not to exceed 10 mg (range of 0.1 μg to 10 mg); preferred 1 μg to 3 mg. The dose per unit area of the device of 0.1 μg-10 μg per mm²; preferred dose of 0.25 μg/mm²-5 μg/mm². Minimum concentration of 10⁻⁸-10⁻⁴ M of paclitaxel is to be maintained on the device surface. (C) Cell cycle inhibitors such as podophyllotoxins (e.g., etoposide): total dose not to exceed 10 mg (range of 0.1 μg to 10 mg); preferred 1 μg to 3 mg. The dose per unit area of the device of 0.1 μg-10 μg per mm²; preferred dose of 0.25 μg/mm²-5 μg/mm². Minimum concentration of 10⁻⁸-10⁻⁴ M of etoposide is to be maintained on the device surface. (D) Immunomodulators including sirolimus and everolimus. Sirolimus (i.e., rapamycin, RAPAMUNE): Total dose not to exceed 10 mg (range of 0.1 μg to 10 mg); preferred 10 μg to 1 mg. The dose per unit area of 0.1 μg-100 μg per mm²; preferred dose of 0.5 μg/mm²-10 μg/mm². Minimum concentration of 10⁻⁸-10⁻⁴ M is to be maintained on the device surface. Everolimus and derivatives and analogues thereof: Total dose should not exceed 10 mg (range of 0.1 μg to 10 mg); preferred 10 μg to 1 mg. The dose per unit area of 0.1 μg-100 μg per mm² of surface area; preferred dose of 0.3 μg/mm²-10 μg/mm². Minimum concentration of 10⁻⁸-10⁻⁴ M of everolimus is to be maintained on the device surface. (E) Heat shock protein 90 antagonists (e.g., geldanamycin) and analogues and derivatives thereof: total dose not to exceed 20 mg (range of 0.1 μg to 20 mg); preferred 1 μg to 5 mg. The dose per unit area of the device of 0.1 μg-10 μg per mm²; preferred dose of 0.25 μg/mm²-5 μg/mm². Minimum concentration of 10⁻⁸-10⁻⁴ M of geldanamycin is to be maintained on the device surface. (F) HMGCoA reductase inhibitors (e.g., simvastatin) and analogues and derivatives thereof: total dose not to exceed 2000 mg (range of 10.0 μg to 2000 mg); preferred 10 μg to 300 mg. The dose per unit area of the device of 1.0 μg-1000 μg per mm²; preferred dose of 2.5 μg/mm²-500 μg/mm². Minimum concentration of 10⁻⁸-10⁻³ M of simvastatin is to be maintained on the device surface. (G) Inosine monophosphate dehydrogenase inhibitors (e.g., mycophenolic acid, 1-alpha-25 dihydroxy vitamin D₃) and analogues and derivatives thereof: total dose not to exceed 2000 mg (range of 10.0 μg to 2000 mg); preferred 10 μg to 300 mg. The dose per unit area of the device of 1.0 μg-1000 μg per mm²; preferred dose of 2.5 μg/mm²-500 μg/mm². Minimum concentration of 10⁻⁸-10⁻³ M of mycophenolic acid is to be maintained on the device surface. (H)NF kappa B inhibitors (e.g., Bay 11-7082) and analogues and derivatives thereof: total dose not to exceed 200 mg (range of 1.0 μg to 200 mg); preferred 1 μg to 50 mg. The dose per unit area of the device of 1.0 μg-100 μg per mm²; preferred dose of 2.5 μg/mm²-50 μg/mm². Minimum concentration of 10⁻⁸-10⁻⁴ M of Bay 11-7082 is to be maintained on the device surface. (I) Antimycotic agents (e.g., sulconizole) and analogues and derivatives thereof: total dose not to exceed 2000 mg (range of 10.0 μg to 2000 mg); preferred 10 μg to 300 mg. The dose per unit area of the device of 1.0 μg-1000 μg per mm²; preferred dose of 2.5 μg/mm²-500 μg/mm². Minimum concentration of 10⁻⁸-10⁻³ M of sulconizole is to be maintained on the device surface. (J) p38 MAP kinase inhibitors (e.g., SB202190) and analogues and derivatives thereof: total dose not to exceed 2000 mg (range of 10.0 μg to 2000 mg); preferred 10 μg to 300 mg. The dose per unit area of the device of 1.0 μg-1000 μg per mm²; preferred dose of 2.5 μg/mm²-500 μg/mm². Minimum concentration of 10⁻⁸-10⁻³ M of SB202190 is to be maintained on the device surface. (K) Anti-angiogenic agents (e.g., halofuginone bromide) and analogues and derivatives thereof: total dose not to exceed 10 mg (range of 0.1 μg to 10 mg); preferred 1 μg to 3 mg. The dose per unit area of the device of 0.1 μg-10 μg per mm²; preferred dose of 0.25 μg/mm²-5 μg/mm². Minimum concentration of 10⁻⁸-10⁻⁴ M of halofuginone bromide is to be maintained on the device surface.

In addition to those described above (e.g., sirolimus, everolimus, and tacrolimus), several other examples of immunomodulators and appropriate dosages ranges for use with spinal devices include the following: (A) Biolimus and derivatives and analogues thereof: Total dose should not exceed 10 mg (range of 0.1 μg to 10 mg); preferred 10 μg to 1 mg. The dose per unit area of 0.1 μg-100 μg per mm² of surface area; preferred dose of 0.3 μg/mm²-10 μg/mm². Minimum concentration of 10⁻⁸-10⁻⁴ M of everolimus is to be maintained on the device surface. (B) Tresperimus and derivatives and analogues thereof: Total dose should not exceed 10 mg (range of 0.1 μg to 10 mg); preferred 10 μg to 1 mg. The dose per unit area of 0.1 μg-100 μg per mm² of surface area; preferred dose of 0.3 μg/mm²-10 μg/mm². Minimum concentration of 10⁻⁸-10⁻⁴ M of tresperimus is to be maintained on the device surface. (C) Auranofin and derivatives and analogues thereof: Total dose should not exceed 10 mg (range of 0.1 μg to 10 mg); preferred 10 μg to 1 mg. The dose per unit area of 0.1 μg-100 μg per mm² of surface area; preferred dose of 0.3 μg/mm²-10 μg/mm². Minimum concentration of 10⁻⁸-10⁻⁴ M of auranofin is to be maintained on the device surface. (D) 27-0-Demethylrapamycin and derivatives and analogues thereof: Total dose should not exceed 10 mg (range of 0.1 μg to 10 mg); preferred 10 μg to 1 mg. The dose per unit area of 0.1 μg-100 μg per mm² of surface area; preferred dose of 0.3 μg/mm²-10 μg/mm². Minimum concentration of 10⁻⁸-10⁻⁴ M of 27-0-Demethylrapamycin is to be maintained on the device surface. (E) Gusperimus and derivatives and analogues thereof: Total dose should not exceed 10 mg (range of 0.1 μg to 10 mg); preferred 10 μg to 1 mg. The dose per unit area of 0.1 μg-100 μg per mm² of surface area; preferred dose of 0.3 μg/mm²-10 μg/mm². Minimum concentration of 10⁻⁸-10⁻⁴ M of gusperimus is to be maintained on the device surface. (F) Pimecrolimus and derivatives and analogues thereof: Total dose should not exceed 10 mg (range of 0.1 μg to 10 mg); preferred 10 μg to 1 mg. The dose per unit area of 0.1 μg-100 μg per mm² of surface area; preferred dose of 0.3 μg/mm²-10 μg/mm². Minimum concentration of 10⁻⁸-10⁻⁴ M of pimecrolimus is to be maintained on the device surface and (G) ABT-578 and analogues and derivatives thereof: Total dose should not exceed 10 mg (range of 0.1 μg to 10 mg); preferred 10 μg to 1 mg. The dose per unit area of 0.1 μg-100 μg per mm² of surface area; preferred dose of 0.3 μg/mm²-10 μg/mm². Minimum concentration of 10⁻⁸-10⁻⁴ M of ABT-578 is to be maintained on the device surface.

In addition to those described above (e.g., paclitaxel, TAXOTERE, and docetaxel), several other examples of anti-microtubule agents and appropriate dosages ranges for use with meshes and films include vinca alkaloids such as vinblastine and vincristine sulfate and analogues and derivatives thereof: total dose not to exceed 10 mg (range of 0.1 μg to 10 mg); preferred 1 μg to 3 mg. Dose per unit area of the device of 0.1 μg-10 μg per mm²; preferred dose of 0.25 μg/mm²-5 μg/mm². Minimum concentration of 10⁻⁸-10⁻⁴ M of drug is to be maintained on the device surface.

It should be apparent to one of skill in the art that potentially any anti-scarring agent described above can be utilized alone, or in combination, in the practice of this embodiment. In various aspects, the present invention provides a medical device contain an angiogenesis inhibitor in a dosage as set forth above. In various aspects, the present invention provides a medical device containing a 5-lipoxygenase inhibitor or antagonist in a dosage as set forth above. In various aspects, the present invention provides a medical device containing a chemokine receptor antagonist in a dosage as set forth above. In various aspects, the present invention provides a medical device containing a cell cycle inhibitor in a dosage as set forth above. In various aspects, the present invention provides a medical device containing an anthracycline (e.g., doxorubicin and mitoxantrone) in a dosage as set forth above. In various aspects, the present invention provides a medical device containing a taxane (e.g., paclitaxel or an analogue or derivative of paclitaxel) in a dosage as set forth above. In various aspects, the present invention provides a medical device containing a podophyllotoxin (e.g., etoposide) in a dosage as set forth above. In various aspects, the present invention provides a medical device containing a vinca alkaloid in a dosage as set forth above. In various aspects, the present invention provides a medical device containing a camptothecin or an analogue or derivative thereof in a dosage as set forth above. In various aspects, the present invention provides a medical device containing a platinum compound in a dosage as set forth above. In various aspects, the present invention provides a medical device containing a nitrosourea in a dosage as set forth above. In various aspects, the present invention provides a medical device containing a nitroimidazole in a dosage as set forth above. In various aspects, the present invention provides a medical device containing a folic acid antagonist in a dosage as set forth above. In various aspects, the present invention provides a medical device containing a cytidine analogue in a dosage as set forth above. In various aspects, the present invention provides a medical device containing a pyrimidine analogue in a dosage as set forth above. In various aspects, the present invention provides a medical device containing a fluoropyrimidine analogue in a dosage as set forth above. In various aspects, the present invention provides a medical device containing a purine analogue in a dosage as set forth above. In various aspects, the present invention provides a medical device containing a nitrogen mustard in a dosage as set forth above. In various aspects, the present invention provides a medical device containing a hydroxyurea in a dosage as set forth above. In various aspects, the present invention provides a medical device containing a mytomicin in a dosage as set forth above. In various aspects, the present invention provides a medical device containing an alkyl sulfonate in a dosage as set forth above. In various aspects, the present invention provides a medical device containing a benzamide in a dosage as set forth above. In various aspects, the present invention provides a medical device containing a nicotinamide in a dosage as set forth above. In various aspects, the present invention provides a medical device containing a halogenated sugar in a dosage as set forth above. In various aspects, the present invention provides a medical device containing a DNA alkylating agent in a dosage as set forth above. In various aspects, the present invention provides a medical device containing an anti-microtubule agent in a dosage as set forth above. In various aspects, the present invention provides a medical device containing a topoisomerase inhibitor in a dosage as set forth above. In various aspects, the present invention provides a medical device containing a DNA cleaving agent in a dosage as set forth above. In various aspects, the present invention provides a medical device containing an antimetabolite in a dosage as set forth above. In various aspects, the present invention provides a medical device containing an agent that inhibits adenosine deaminase in a dosage as set forth above. In various aspects, the present invention provides a medical device containing an agent that inhibits purine ring synthesis in a dosage as set forth above. In various aspects, the present invention provides a medical device containing a nucleotide interconversion inhibitor in a dosage as set forth above. In various aspects, the present invention provides a medical device containing an agent that inhibits dihydrofolate reduction in a dosage as set forth above. In various aspects, the present invention provides a medical device containing an agent that blocks thymidine monophosphate in a dosage as set forth above. In various aspects, the present invention provides a medical device containing an agent that causes DNA damage in a dosage as set forth above. In various aspects, the present invention provides a medical device containing a DNA intercalation agent in a dosage as set forth above. In various aspects, the present invention provides a medical device containing an agent that is a RNA synthesis inhibitor in a dosage as set forth above. In various aspects, the present invention provides a medical device containing an agent that is a pyrimidine synthesis inhibitor in a dosage as set forth above. In various aspects, the present invention provides a medical device containing an agent that inhibits ribonucleotide synthesis in a dosage as set forth above. In various aspects, the present invention provides a medical device containing an agent that inhibits thymidine monophosphate function in a dosage as set forth above. In various aspects, the present invention provides a medical device containing an agent that inhibits DNA synthesis in a dosage as set forth above. In various aspects, the present invention provides a medical device containing an agent that causes DNA adduct formation in a dosage as set forth above. In various aspects, the present invention provides a medical device containing an agent that inhibits protein synthesis in a dosage as set forth above. In various aspects, the present invention provides a medical device containing an agent that inhibits microtubule function in a dosage as set forth above. In various aspects, the present invention provides a medical device containing an immunomodulatory agent (e.g., sirolimus, everolimus, tacrolimus, or an analogue or derivative thereof) in a dosage as set forth above. In various aspects, the present invention provides a medical device containing a heat shock protein 90 antagonist (e.g., geldanamycin) in a dosage as set forth above. In various aspects, the present invention provides a medical device containing an HMGCoA reductase inhibitor (e.g., simvastatin) in a dosage as set forth above. In various aspects, the present invention provides a medical device containing an inosine monophosphate dehydrogenase inhibitor (e.g., mycophenolic acid, 1-alpha-25 dihydroxy vitamin D₃) in a dosage as set forth above. In various aspects, the present invention provides a medical device containing an NF kappa B inhibitor (e.g., Bay 11-7082) in a dosage as set forth above. In various aspects, the present invention provides a medical device containing an antimycotic agent (e.g., sulconizole) in a dosage as set forth above. In various aspects, the present invention provides a medical device containing a p38 MAP Kinase inhibitor (e.g., SB202190) in a dosage as set forth above. In various aspects, the present invention provides a medical device containing a cyclin dependent protein kinase inhibitor in a dosage as set forth above. In various aspects, the present invention provides a medical device containing an epidermal growth factor kinase inhibitor in a dosage as set forth above. In various aspects, the present invention provides a medical device containing an elastase inhibitor in a dosage as set forth above. In various aspects, the present invention provides a medical device containing a factor Xa inhibitor in a dosage as set forth above. In various aspects, the present invention provides a medical device containing a farnesyltransferase inhibitor in a dosage as set forth above. In various aspects, the present invention provides a medical device containing a fibrinogen antagonist in a dosage as set forth above. In various aspects, the present invention provides a medical device containing a guanylate cyclase stimulant in a dosage as set forth above. In various aspects, the present invention provides a medical device containing a hydroorotate dehydrogenase inhibitor in a dosage as set forth above. In various aspects, the present invention provides a medical device containing an IKK2 inhibitor in a dosage as set forth above. In various aspects, the present invention provides a medical device containing an IL-1 antagonist in a dosage as set forth above. In various aspects, the present invention provides a medical device containing an ICE antagonist in a dosage as set forth above. In various aspects, the present invention provides a medical device containing an IRAK antagonist in a dosage as set forth above. In various aspects, the present invention provides a medical device containing an IL-4 agonist in a dosage as set forth above. In various aspects, the present invention provides a medical device containing a leukotriene inhibitor in a dosage as set forth above. In various aspects, the present invention provides a medical device containing an MCP-1 antagonist in a dosage as set forth above. In various aspects, the present invention provides a medical device containing a MMP inhibitor in a dosage as set forth above. In various aspects, the present invention provides a medical device containing an NO agonist in a dosage as set forth above. In various aspects, the present invention provides a medical device containing a phosphodiesterase inhibitor in a dosage as set forth above. In various aspects, the present invention provides a medical device containing a TGF beta inhibitor in a dosage as set forth above. In various aspects, the present invention provides a medical device containing a thromboxane A2 antagonist in a dosage as set forth above. In various aspects, the present invention provides a medical device containing a TNFa antagonist in a dosage as set forth above. In various aspects, the present invention provides a medical device containing a TACE inhibitor in a dosage as set forth above. In various aspects, the present invention provides a medical device containing a tyrosine kinase inhibitor in a dosage as set forth above. In various aspects, the present invention provides a medical device containing a vitronectin inhibitor in a dosage as set forth above. In various aspects, the present invention provides a medical device containing a fibroblast growth factor inhibitor in a dosage as set forth above. In various aspects, the present invention provides a medical device containing a protein kinase inhibitor in a dosage as set forth above. In various aspects, the present invention provides a medical device containing a PDGF receptor kinase inhibitor in a dosage as set forth above. In various aspects, the present invention provides a medical device containing an endothelial growth factor receptor kinase inhibitor in a dosage as set forth above. In various aspects, the present invention provides a medical device containing a retinoic acid receptor antagonist in a dosage as set forth above. In various aspects, the present invention provides a medical device containing a platelet derived growth factor receptor kinase inhibitor in a dosage as set forth above. In various aspects, the present invention provides a medical device containing a fibronogin antagonist in a dosage as set forth above. In various aspects, the present invention provides a medical device containing a bisphosphonate in a dosage as set forth above. In various aspects, the present invention provides a medical device containing a phospholipase A1 inhibitor in a dosage as set forth above. In various aspects, the present invention provides a medical device containing a histamine H1/H2/H3 receptor antagonist in a dosage as set forth above. In various aspects, the present invention provides a medical device containing a macrolide antibiotic in a dosage as set forth above. In various aspects, the present invention provides a medical device containing a GPIIb IIIa receptor antagonist in a dosage as set forth above. In various aspects, the present invention provides a medical device containing an endothelin receptor antagonist in a dosage as set forth above. In various aspects, the present invention provides a medical device containing a peroxisome proliferator-activated receptor agonist in a dosage as set forth above. In various aspects, the present invention provides a medical device containing an estrogen receptor agent in a dosage as set forth above. In various aspects, the present invention provides a medical device containing a somastostatin analogue in a dosage as set forth above. In various aspects, the present invention provides a medical device containing a neurokinin 1 antagonist in a dosage as set forth above. In various aspects, the present invention provides a medical device containing a neurokinin 3 antagonist in a dosage as set forth above. In various aspects, the present invention provides a medical device containing a VLA-4 antagonist in a dosage as set forth above. In various aspects, the present invention provides a medical device containing an osteoclast inhibitor in a dosage as set forth above. In various aspects, the present invention provides a medical device containing a DNA topoisomerase ATP hydrolyzing inhibitor in a dosage as set forth above. In various aspects, the present invention provides a medical device containing an angiotensin I converting enzyme inhibitor in a dosage as set forth above. In various aspects, the present invention provides a medical device containing an angiotensin II antagonist in a dosage as set forth above. In various aspects, the present invention provides a medical device containing an enkephalinase inhibitor in a dosage as set forth above. In various aspects, the present invention provides a medical device containing a peroxisome proliferator-activated receptor gamma agonist insulin sensitizer in a dosage as set forth above. In various aspects, the present invention provides a medical device containing a protein kinase C inhibitor in a dosage as set forth above.

The following examples are offered by way of illustration, and not by way of limitation.

### EXAMPLES

### Example 1

### Parylene Coating

The metallic portion of a coronary stent is washed by dipping it into HPLC grade isopropanol. The cleaned device is then coated with a parylene coating using a parylene coater and either di-p-xylylene or dichloro-di-p-xylylene as the coating feed material. This procedure may be used to coat other types of medical devices that include a metallic portion (e.g., peripheral stents, covered stents, guidewires, shunts, GI drainage tubes, and anastomotic connectors).

### Example 2

Paclitaxel Coating—End Coating

Paclitaxel solutions are prepared by dissolving paclitaxel in 5 mL HPLC grade THF. The ends of a parylene coated coronary stent (prepared as in Example 1) are then dipped into the paclitaxel/THF solution. After various incubation times, the devices are removed and dried in a forced air oven (50° C.). The device is then further dried in a vacuum oven overnight. The amount of paclitaxel used in each solution is varied such that the amount of paclitaxel coated onto the ends of the device is in the range of 0.06 mg/mm² to 10 mg/mm². In addition to paclitaxel, the following are exemplary compounds that may be used to coat the device: mitoxantrone, doxorubicin, epithilone B, etoposide, TAXOTERE, tubercidin, vinblastine, geldanamycin, simvastatin, sirolimus, everolimus, mycophenolic acid, 1-alpha-25 dihydroxy vitamin D₃, Bay 11-7082, SB202190, and sulconizole. This procedure may be used to coat other types of devices that include a metallic portion (e.g., peripheral stents, covered stents, guidewires, GI drainage tubes, shunts, and anastomotic connectors).

### Example 3

### Paclitaxel Coating—Complete Coating

Paclitaxel solutions are prepared by dissolving paclitaxel in 5 mL HPLC grade THF. A parylene coated coronary stent (as prepared in Example 1) is then dipped entirely into the paclitaxel/THF solution. After various incubation times, the device is removed and dried in a forced air oven (50° C.). The device is then further dried in a vacuum oven overnight. The amount of paclitaxel used in each solution is varied such that the amount of paclitaxel coated onto the ends of the device is in the range of 0.06 mg/mm² to 10 mg/mm². In addition to paclitaxel, the following are exemplary compounds that may be also used to coat the device: paclitaxel, mitoxantrone, doxorubicin, epithilone B, etoposide, TAXOTERE, tubercidin, vinblastine, geldanamycin, simvastatin, sirolimus, everolimus, mycophenolic acid, 1-alpha-25 dihydroxy vitamin D₃, Bay 11-7082, SB202190, and sulconizole. This procedure may be used to coated other types of paryiene coated devices that include a metallic portion (e.g., peripheral stents, covered stents, guidewires, GI drainage tubes, shunts, and anastomotic connectors).

### Example 4

### Application of a Parylene Overcoat

A paclitaxel coated device is placed in a parylene coater and an additional thin layer of parylene is deposited on the paclitaxel coated device (see Examples 2 or 3). The coating duration is altered such that the parylene top-coat thickness is varied such that different elution profiles of the paclitaxel may be obtained.

### Example 5

### Application of an Echogenic Coating Layer

DESMODUR (Bayer AG, Germany), an isocyanate pre-polymer, is dissolved in a 50:50 mixture of dimethylsulfoxide and tetrahydrofuran. A paclitaxel/parylene overcoated coronary stent (prepared as in Example 4) is then dipped into the pre-polymer solution. The device is then removed and the coating is then partially dried at room temperature for 3 to 5 minutes. The device is then immersed in a beaker of water (room temperature) for 3-5 minutes to cause the polymerization reaction to occur rapidly. An echogenic coating is formed. This procedure may be used to coat other types of devices (e.g., peripheral stents, covered stents, guidewires, GI drainage tubes, shunts, and anastomotic connectors).

### Example 6

### Paclitaxel/Polymer Coating—End Coating

5% solutions of poly(ethylene-co-vinyl acetate) (EVA) (60% vinyl acetate) are prepared using THF as the solvent. Various amounts of paclitaxel are added to each of the EVA solutions. The ends of a corornary stent are dipped into the paclitaxel/EVA solution. After removing the end-coated device from the solution, the coating is dried by placing the device in a forced air oven (40° C.) for 3 hours. The coated device is then further dried under vacuum for 24 hours. The dip coating process may be repeated to increase the amount of polymer/paclitaxel coated onto the device. In addition to paclitaxel, the following are exemplary compounds that may also be used to coat the device: mitoxantrone, doxorubicin, epithilone B, etoposide, TAXOTERE, tubercidin, vinblastine, geldanamycin, simvastatin, sirolimus, everolimus, mycophenolic acid, 1-alpha-25 dihydroxy vitamin D₃, Bay 11-7082, SB202190, and sulconizole. This procedure may be used to coated other types of devices (e.g., central venous catheters, ventricular assist devices, peripheral stents, and nasal stents).

### Example 7

### Paclitaxel-Heparin Coating—End Coating

5% solutions of poly(ethylene-co-vinyl acetate) (EVA) (60% vinyl acetate) are prepared using THF as the solvent. Various amounts of paclitaxel and a solution of tridodecyl methyl ammonium chloride-heparin complex (PolySciences) are added to each of the EVA solutions. The ends of an anastomotic connector device are dipped into the paclitaxel/EVA solution. After removing the end-coated device from the solution, the coating is dried by placing the anastomotic device in a forced air oven (40° C.) for 3 hours. The coated anastomotic device is then further dried under vacuum for 24 hours. In addition to paclitaxel, the following are exemplary compounds that may be used to coat the device: mitoxantrone, doxorubicin, epithilone B, etoposide, TAXOTERE, tubercidin, vinblastine, geldanamycin, simvastatin, sirolimus, everolimus, mycophenolic acid, 1-alpha-25 dihydroxy vitamin D₃, Bay 11-7082, SB202190, and sulconizole. This procedure may be used to coated other types of devices including peritoneal dialysis catheters, coronary stents, peripheral stents, hemodialysis access devices, guidewires, shunts, and VAD's.

### Example 8

### Paclitaxel—Heparin/Heparin Coating

The uncoated portions of paclitaxel-heparin coated devices (Example 7) are dipped into a 5% EVA solution containing different amounts of a tridodecyl methyl ammonium chloride-heparin complex solution (PolySciences). After removing the end-coated device from the solution, the coating is dried by placing the anastomotic device in a forced air oven (40° C.) for 3 hours. The coated device is then further dried under vacuum for 24 hours. This provides a device with a paclitaxel/heparin coating on the ends of the device and a heparin coating on the remaining parts of the device. In addition to paclitaxel, the following are exemplary compounds that may be used to coat the device: mitoxantrone, doxorubicin, epithilone B, etoposide, TAXOTERE, tubercidin, vinblastine, geldanamycin, simvastatin, sirolimus, everolimus, mycophenolic acid, 1-alpha-25 dihydroxy vitamin D₃, Bay 11-7082, SB202190, and sulconizole. This procedure may be used to coated other types of devices including peritoneal dialysis catheters, coronary stents, peripheral stents, hemodialysis access devices, guidewires, shunts, and VAD's.

### Example 9

### Paclitaxel/Polymer Coating—End Coating

5% solutions of poly(styrene-co-isobutylene-styrene) (SIBS) are prepared using THF as the solvent. Various amounts of paclitaxel are added to each of the SIBS solutions. The ends of a central venous catheter device are dipped into the paclitaxel/SIBS solution. After removing the end-coated device from the solution, the coating is dried by placing the device in a forced air oven (40° C.) for 3 hours. The coated device is then further dried under vacuum for 24 hours. The dip coating process may be repeated to increase the amount of polymer/paclitaxel coated onto the device. In addition to paclitaxel, the following exemplary compounds that may be used to coat the device: mitoxantrone, doxorubicin, epithilone B, etoposide, TAXOTERE, tubercidin, vinblastine, geldanamycin, simvastatin, sirolimus, everolimus, mycophenolic acid, 1-alpha-25 dihydroxy vitamin D₃, Bay 11-7082, SB202190, and sulconizole. This procedure may be used to coated other types of devices including peritoneal dialysis catheters, coronary stents, non-vascular stents, peripheral stents, hemodialysis access devices, guidewires, shunts, and anastomotic connectors, LVAD's.

### Example 10

### Paclitaxel/Polymer Coating—Echogenic Overcoat

A coated CVC device from Example 9 is dipped into a DESMODUR solution (50:50 mixture of dimethylsulfoxide and tetrahydrofuran). The anastomotic device is then removed and the coating is then partially dried at room temperature for 3 to 5 minutes. The device is then immersed in a beaker of water (room temperature) for 3-5 minutes to cause the polymerization reaction to occur rapidly. An echogenic coating is formed. In addition to paclitaxel, the following are exemplary compounds that may be used to coat the device: mitoxantrone, doxorubicin, epithilone B, etoposide, TAXOTERE, tubercidin, vinblastine, geldanamycin, simvastatin, sirolimus, everolimus, mycophenolic acid, 1-alpha-25 dihydroxy vitamin D₃, Bay 11-7082, SB202190, and sulconizole.

### Example 11

### Polymer/Echogenic Coating

5% solutions of poly(styrene-co-isobutylene-styrene) (SIBS) are prepared using THF as the solvent. A LVAD device is dipped into the SIBS solution. After removing the device from the solution, the coating is dried by placing the device in a forced air oven (40° C.) for 3 hours. The coated device is then further dried under vacuum for 24 hours.

The coated device is dipped into a DESMODUR solution (50:50 mixture of dimethylsulfoxide and tetrahydrofuran). The device is then removed and the coating is then partially dried at room temperature for 3 to 5 minutes. The device is then immersed in a beaker of water (room temperature) for 3-5 minutes to cause the polymerization reaction to occur rapidly. The device is dried under vacuum for 24 hours at room temperature. The ends of the coated device are immersed into a solution of paclitaxel. The device is removed and dried at 40° C. for 1 hour and then under vacuum for 24 hours.

The amount of paclitaxel absorbed by the polymeric coating may be altered by changing the paclitaxel concentration, the immersion time as well as the solvent composition of the paclitaxel solution. In addition to paclitaxel, the following are exemplary compounds that may be used to coat the device: mitoxantrone, doxorubicin, epithilone B, etoposide, TAXOTERE, tubercidin, vinblastine, geldanamycin, simvastatin, sirolimus, everolimus, mycophenolic acid, 1-alpha-25 dihydroxy vitamin D₃, Bay 11-7082, SB202190, and sulconizole.

This procedure may be used to coat other types of devices including peritoneal dialysis catheters, coronary stents, non-vascular stents, peripheral stents, hemodialysis access devices, guidewires, shunts, anastomotic connectors, CVC's.

### Example 12

### Paclitaxel/Siloxane Coating—End Coating

A central venous catheter is coated with a silioxane layer by exposing the device to gaseous tetramethylcyclotetrasiloxane that is then polymerized by low energy plasma polymerization onto the device surface. The thickness of the siloxane layer may be increased by increasing the polymerization time. The ends of the device are then immersed into a paclitaxel/THF solution. The paclitaxel is absorbed into the siloxane coating. The device is then removed from the solution and is dried for 2 hours at 40° C. in a forced air oven. The device is then further dried under vacuum at room temperature for 24 hours. The amount of paclitaxel coated onto the device ends may be varied by altering the concentration of the paclitaxel/THF solution as well as altering the immersion time of the device ends in the paclitaxel THF solution. In addition to paclitaxel, the following are exemplary compounds that may be used to coat the device: mitoxantrone, doxorubicin, epithilone B, etoposide, TAXOTERE, tubercidin, vinblastine, geldanamycin, simvastatin, sirolimus, everolimus, mycophenolic acid, 1-alpha-25 dihydroxy vitamin D₃, Bay 11-7082, SB202190, and sulconizole. This procedure may be used to coat other types of devices including peritoneal dialysis catheters, coronary stents, non-vascular stents, peripheral stents, hemodialysis access devices, guidewires, GI drainage tubes, shunts, and anastomotic connectors.

### Example 13

### Heparin Coating

A CNS shunt device is dipped into a solution containing different amounts of a tridodecyl methyl ammonium chloride-heparin complex solution (PolySciences). After various incubation times, the device is removed and dried in a forced air oven (50° C.). The device is then further dried in a vacuum oven overnight. Other types of devices that may be coated with this procedure include coronary stents, peripheral stents, nasal and sinus stents, tracheal stents, peritoneal dialysis catheters, vascular grafts, hemodialysis access devices, guidewires, shunts, and anastomotic connectors.

### Example 14

### Spray-Coated Devices

2% solutions poly(styrene-co-isobutylene-styrene) (SIBS) are prepared using THF as the solvent. Various amounts of paclitaxel are added to each solution. A device (e.g., a stent, central venous catheter, LVAD, anastomotic connector, or shunt) is held with a pair of tweezers and is then spray coated with one of the paclitaxel/polymer solutions using an airbrush. The device is then air-dried. The device is then held in a new location using the tweezers and a second coat of paclitaxel/polymer is applied. The device is air-dried and is then dried under vacuum overnight. The total amount of paclitaxel coated onto the device may be altered by changing the paclitaxel content in the solution as well as by increasing the number of coatings applied. In addition to paclitaxel, the following are exemplary compounds that may be used to coat the device: mitoxantrone, doxorubicin, epithilone B, etoposide, TAXOTERE, tubercidin, vinblastine, geldanamycin, simvastatin, sirolimus, everolimus, mycophenolic acid, 1-alpha-25 dihydroxy vitamin D₃, Bay 11-7082, SB202190, and sulconizole.

### Example 15

### Drug Coated Covered Stent-Non-Degradable

A covered stent (WALLGRAFT, Boston Scientific Corporation) is attached to a rotating mandrel. A solution of paclitaxel (5% w/w) in a polyurethane (CHRONOFLEX 85A)/THF solution (2.5% w/v) is then sprayed onto the outer surface of the covered stent. The solution is sprayed on at a rate that ensures that the graft material is not damaged or saturated with the sprayed solution. The covered stent is allowed to air dry after which it is dried under vacuum for 24 hours. In addition to paclitaxel, the following are exemplary compounds that may be used to coat the device: mitoxantrone, doxorubicin, epithilone B, etoposide, TAXOTERE, tubercidin, vinblastine, geldanamycin, simvastatin, sirolimus, everolimus, mycophenolic acid, 1-alpha-25 dihydroxy vitamin D₃, Bay 11-7082, SB202190, and sulconizole.

### Example 16

### Drug Coated Covered Stent—Degradable

A WALLGRAFT stent is attached to a rotating mandrel. Paclitaxel (5% w/w) in a PLGA/ethyl acetate solution (2.5% w/v) is then sprayed onto the outer surface of the covered stent. The solution is sprayed on at a rate that ensures that the graft material is not damaged or saturated with the sprayed solution. The covered stent is allowed to air dry after which it is dried under vacuum for 24 hours. In addition to paclitaxel, the following are exemplary compounds that may also be used to coat the device: paclitaxel, mitoxantrone, doxorubicin, epithilone B, etoposide, TAXOTERE, tubercidin, vinblastine, geldanamycin, simvastatin, sirolimus, everolimus, mycophenolic acid, 1-alpha-25 dihydroxy vitamin D₃, Bay 11-7082, SB202190, and sulconizole.

### Example 17

### Drug Coated Covered Stent—Degradable Overcoat

A drug-coated WALLGRAFT stent from either Example 15 or Example 16 is attached to a rotating mandrel. A PLGA/ethyl acetate solution (2.5% w/v) is then sprayed onto the outer surface of the covered stent such that a coating is formed over the initial drug containing coating. The solution is sprayed on at a rate that ensures that the graft material is not damaged or saturated with the sprayed solution. The covered stent is allowed to air dry after which it is dried under vacuum for 24 hours.

### Example 18

### Drug-Loaded Microsphere Formulation

Paclitaxel (10% w/w) is added to a solution of PLGA (50/50, Mw≈54,000) in DCM (5% w/v). The solution is vortexed and then poured into a stirred (overhead stirrer with a 3 bladed TEFLON coated stirrer) aqueous PVA (approximately 89% hydrolyzed, Mw≈13,000, 2% w/v). The solution is stirred for 6 hours after which the solution is centrifuged to sediment the microspheres. The microspheres were resuspended in water. The centrifugation—washing process is repeated 4 times. The final microsphere solution is flash frozen in an acetone/dry-ice bath. The frozen solution is then freeze-dried to produce a fine powder. The size of the microspheres formed may be altered by changing the stirring speed and/or the PVA solution concentration. The freeze dried powder may be resuspended in PBS or saline and may be used for direct injection, as an incubation fluid or as an irrigation fluid. In addition to paclitaxel, the following are exemplary compounds that may be used to coat the device: mitoxantrone, doxorubicin, epithilone B, etoposide, TAXOTERE, tubercidin, vinblastine, geldanamycin, simvastatin, sirolimus, everolimus, mycophenolic acid, 1-alpha-25 dihydroxy vitamin D3, Bay 11-7082, SB202190, and sulconizole.

### Example 19

### Drug Coated Stent (Exterior Coating)

A stent is dipped into a polyurethane (CHRONOFLEX 85A)/THF solution (2.5% w/v). The coated stent is allowed to air dry for 10 seconds. The stent is then rolled in powdered paclitaxel that is spread thinly on a piece of release liner. The rolling process is done in such a manner that the paclitaxel powder predominantly adheres to the exterior side of the coated stent. The stents are air-dried for 1 hour followed by vacuum drying for 24 hours. In addition to paclitaxel, the following are exemplary compounds that may be used to coat the device: mitoxantrone, doxorubicin, epithilone B, etoposide, TAXOTERE, tubercidin, vinblastine, geldanamycin, simvastatin, sirolimus, everolimus, mycophenolic acid, 1-alpha-25 dihydroxy vitamin D₃, Bay 11-7082, SB202190, and sulconizole.

### Example 20

### Drug Coated Stent (Exterior Coating) with a Heparin Coating

The drug-coated stent from Example 19 is further coated with a heparin coating. The stents that are prepared in Example 19 are dipped into a solution of heparin-benzalkonium chloride complex (1.5% (w/v) in isopropanol, STS Biopolymers). The stents are removed from the solution and are air-dried for 1 hour followed by vacuum drying for 24 hours. This process results in both the interior and exterior surfaces of the covered stent being coated with heparin.

### Example 21

### Partial Drug Coating of a Covered Stent

A WALLGRAFT covered stent is attached to a rotating mandrel. A mask system is set up so that only the middle of the outer surface of the covered stent may be sprayed. A solution of paclitaxel (5% w/w) in a polyurethane (CHRONOFLEX 85A)/THF solution (2.5% w/v) is then sprayed onto the outer surface of the covered stent. The solution is sprayed on at a rate that ensures that the graft material is not damaged or saturated with the sprayed solution. The covered stent is allowed to air dry after which it is dried under vacuum for 24 hours. In addition to paclitaxel, the following are exemplary compounds that also may be used to coat the device: mitoxantrone, doxorubicin, epithilone B, etoposide, TAXOTERE, tubercidin, vinblastine, geldanamycin, simvastatin, sirolimus, everolimus, mycophenolic acid, 1-alpha-25 dihydroxy vitamin D₃, Bay 11-7082, SB202190, and sulconizole.

### Example 22

### Drug—Dexamethasone Coated Covered Stent

A WALLGRAFT covered stent is attached to a rotating mandrel. A mask system is set up so that only the middle of the outer surface of the covered stent may be sprayed. A solution of paclitaxel (5% w/w) in a polyurethane (CHRONOFLEX 85A)/THF solution (2.5% w/v) is then sprayed onto the outer surface of the covered stent. The solution is sprayed on at a rate that ensures that the graft material is not damaged or saturated with the sprayed solution. The covered stent is allowed to air dry. The mask is then rearranged so that only the ends of the outer surface of the covered stent may be sprayed. The ends of the outer surface of the covered stent are then sprayed with a dexamethasone (10% w/w)/polyurethane (CHRONOFLEX 85A)/THF solution (2.5% w/v). The sample is air dried after which it is dried under vacuum for 24 hours. In addition to paclitaxel, the following are exemplary compounds that also may be used to coat the device: mitoxantrone, doxorubicin, epithilone B, etoposide, TAXOTERE, tubercidin, vinblastine, geldanamycin, simvastatin, sirolimus, everolimus, mycophenolic acid, 1-alpha-25 dihydroxy vitamin D₃, Bay 11-7082, SB202190, and sulconizole.

### Example 23

### Drug—Heparin Coated Covered Stent

A WALLGRAFT covered stent is attached to a rotating mandrel. A mask system is set up so that only the middle of the outer surface of the covered stent may be sprayed. A solution of paclitaxel (5% w/w) in a polyurethane (CHRONOFLEX 85A)/THF solution (2.5% w/v) is then sprayed onto the outer surface of the covered stent. The solution is sprayed on at a rate that ensures that the graft material is not damaged or saturated with the sprayed solution. The covered stent is allowed to air dry. The mask is then rearranged so that only the ends of the outer surface of the covered stent may be sprayed. The ends of the outer surface of the covered stent are then sprayed with a heparin-benzalkonium chloride complex (1.5% (w/v) in isopropanol, STS Biopolymers). The sample is air dried after which it is dried under vacuum for 24 hours. In addition to paclitaxel, the following are exemplary compounds that may be used to coat the device: mitoxantrone, doxorubicin, epithilone B, etoposide, TAXOTERE, tubercidin, vinblastine, geldanamycin, simvastatin, sirolimus, everolimus, mycophenolic acid, 1-alpha-25 dihydroxy vitamin D₃, Bay 11-7082, SB202190, and sulconizole.

### Example 24

### Drug-Dexamethaxone Coated Covered Stent

A WALLGRAFT stent is attached to a rotating mandrel. A solution of paclitaxel (5% w/w) and dexamethazone (5% w/w) in a PLGA (50/50, Mw^{˜b 54,000})/ethyl acetate solution (2.5% w/v) is sprayed onto the outer surface of the covered stent. The solution is sprayed on at a rate that ensures that the graft material is not damaged or saturated with the sprayed solution. The covered stent is allowed to air dry after which it is dried under vacuum for 24 hours. In addition to paclitaxel, the following are exemplary compounds that also may be used to coat the device: paclitaxel, mitoxantrone, doxorubicin, epithilone B, etoposide, TAXOTERE, tubercidin, vinblastine, geldanamycin, simvastatin, sirolimus, everolimus, mycophenolic acid, 1-alpha-25 dihydroxy vitamin D₃, Bay 11-7082, SB202190, and sulconizole.

### Example 25

### Drug-Dexamethasone Coated Covered Stent (Sequential Coating)

A WALLGRAFT stent is attached to a rotating mandrel. A solution of paclitaxel (5% w/w) in a PLGA (50/50, Mw^{˜}54,000)/ethyl acetate solution (2.5% w/v) is sprayed onto the outer surface of the covered stent. The solution is sprayed on at a rate that ensures that the graft material is not damaged or saturated with the sprayed solution. The covered stent is allowed to air dry. A methanol solution of dexamethasone is then sprayed onto the outer surface of the covered stent (at a rate that ensures that the graft material is not damaged or saturated with the sprayed solution). The covered stent is allowed to air dry after which it is dried under vacuum for 24 hours. In addition to paclitaxel, the following are exemplary compounds that may be used to coat the device: mitoxantrone, doxorubicin, epithilone B, etoposide, TAXOTERE, tubercidin, vinblastine, geldanamycin, simvastatin, sirolimus, everolimus, mycophenolic acid, 1-alpha-25 dihydroxy vitamin D₃, Bay 11-7082, SB202190, and sulconizole.

### Example 26

### Screening Assay for Assessing the Effect of Various Compounds on Nitric Oxide Production by Macrophages

The murine macrophage cell line RAW 264.7 was trypsinized to remove cells from flasks and plated in individual wells of a 6-well plate. Approximately 2×10⁶ cells were plated in 2 mL of media containing 5% heat-inactivated fetal bovine serum (FBS). RAW 264.7 cells were incubated at 37° C. for 1.5 hours to allow adherence to plastic. Mitoxantrone was prepared in DMSO at a concentration of 10⁻² M and serially diluted 10-fold to give a range of stock concentrations (10⁻⁸ M to 10⁻² M). Media was then removed and cells were incubated in 1 ng/mL of recombinant murine IFN? and 5 ng/mL of LPS with or without mitoxantrone in fresh media containing 5% FBS. Mitoxantrone was added to cells by directly adding mitoxantrone DMSO stock solutions, prepared earlier, at a 1/1000 dilution, to each well. Plates containing IFN?, LPS plus or minus mitoxantrone were incubated at 37° C. for 24 hours (Chem. Ber. (1879) 12: 426; J. AOAC (1977) 60-594; Ann. Rev. Biochem. (1994) 63: 175).

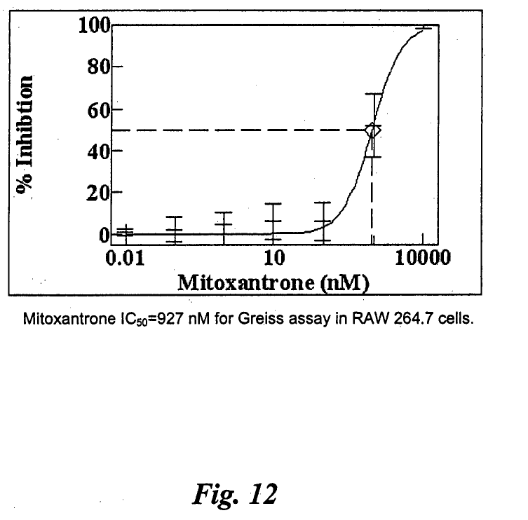

At the end of the 24 hour period, supernatants were collected from the cells and assayed for the production of nitrites. Each sample was tested in triplicate by aliquoting 50 μL of supernatant in a 96-well plate and adding 50 μL of Greiss Reagent A (0.5 g sulfanilamide, 1.5 mL H₃PO₄, 48.5 mL ddH₂O) and 50 μL of Greiss Reagent B (0.05 g N-(1-naphthyl)-ethylenediamine, 1.5 mL H₃PO₄, 48.5 mL ddH₂O). Optical density was read immediately on microplate spectrophotometer at 562 nm absorbance. Absorbance over triplicate wells was averaged after subtracting background and concentration values were obtained from the nitrite standard curve (1 μM to 2 mM). Inhibitory concentration of 50% (IC₅₀) was determined by comparing average nitrite concentration to the positive control (cell stimulated with IFN? and LPS). An average of n=4 replicate experiments was used to determine IC₅₀ values for mitoxantrone (see, FIG. 12 (IC₅₀=927 nM)). The IC₅₀ values for the following additional compounds were determined using this assay: IC₅₀ (nM): paclitaxel, 7; CNI-1493, 249; halofuginone, 12; geldanamycin, 51; anisomycin, 68; 17-AAG, 840; epirubicin hydrochloride, 769.

### Example 27

### Screening Assay for Assessing the Effect of Various Anti-Scarring Agents on Tnf-Alpha Production by Macrophages

The human macrophage cell line, THP-1 was plated in a 12 well plate such that each well contains 1×10⁶ cells in 2 mL of media containing 10% FCS. Opsonized zymosan was prepared by resuspending 20 mg of zymosan A in 2 mL of ddH₂O and homogenizing until a uniform suspension was obtained. Homogenized zymosan was pelleted at 250 g and resuspended in 4 mL of human serum for a final concentration of 5 mg/mL and incubated in a 37° C. water bath for 20 minutes to enable opsonization. Bay 11-7082 was prepared in DMSO at a concentration of 10⁻² M and serially diluted 10-fold to give a range of stock concentrations (10⁻⁸ M to 10⁻² M) (J. Immunol. (2000) 165: 411-418; J. Immunol. (2000) 164: 4804-4811; J. Immunol Meth. (2000) 235 (1-2): 33-40).

THP-1 cells were stimulated to produce TNFa by the addition of 1 mg/mL opsonized zymosan. Bay 11-7082 was added to THP-1 cells by directly adding DMSO stock solutions, prepared earlier, at a 1/1000 dilution, to each well. Each drug concentration was tested in triplicate wells. Plates were incubated at 37° C. for 24 hours.

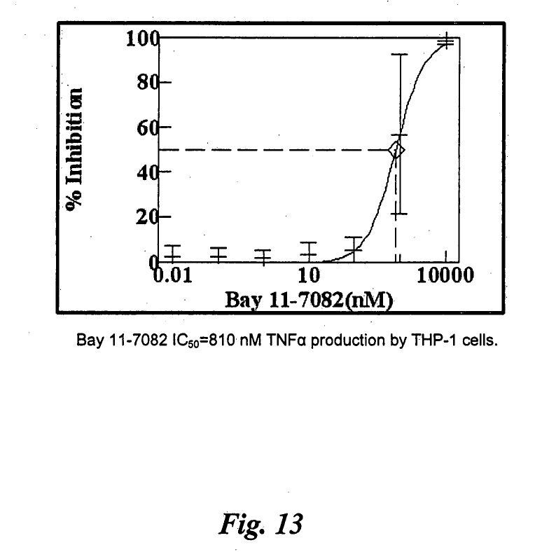

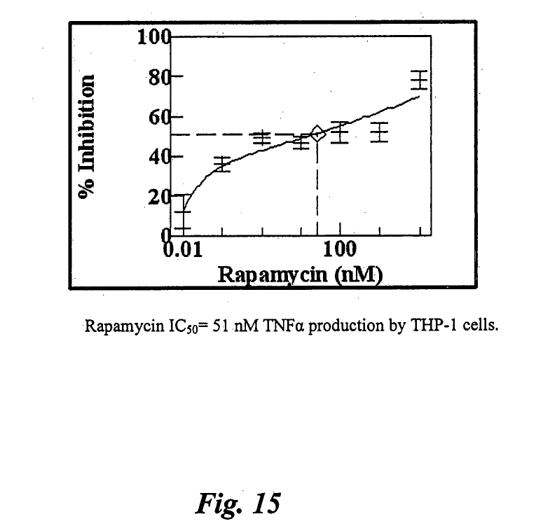

After a 24 hour stimulation, supernatants were collected to quantify TNFa production. TNFa concentrations in the supernatants were determined by ELISA using recombinant human TNFa to obtain a standard curve. A 96-well MaxiSorb plate was coated with 100 μL of anti-human TNFa Capture Antibody diluted in Coating Buffer (0.1M sodium carbonate pH 9.5) overnight at 4° C. The dilution of Capture Antibody used was lot-specific and was determined empirically. Capture antibody was then aspirated and the plate washed 3 times with Wash Buffer (PBS, 0.05% TWEEN-20). Plates were blocked for 1 hour at room temperature with 200 μL/well of Assay Diluent (PBS, 10% FCS pH 7.0). After blocking, plates were washed 3 times with Wash Buffer. Standards and sample dilutions were prepared as follows: (a) sample supernatants were diluted 1/8 and 1/16; (b) recombinant human TNFa was prepared at 500 pg/mL and serially diluted to yield as standard curve of 7.8 pg/mL to 500 pg/mL. Sample supernatants and standards were assayed in triplicate and were incubated at room temperature for 2 hours after addition to the plate coated with Capture Antibody. The plates were washed 5 times and incubated with 100 μL of Working Detector (biotinylated anti-human TNFa detection antibody+avidin-HRP) for 1 hour at room temperature. Following this incubation, the plates were washed 7 times and 100 μL of Substrate Solution (tetramethylbenzidine, H₂O₂) was added to plates and incubated for 30 minutes at room temperature. Stop Solution (2 N H₂SO₄) was then added to the wells and a yellow color reaction was read at 450 nm with ? correction at 570 nm. Mean absorbance was determined from triplicate data readings and the mean background was subtracted. TNFa concentration values were obtained from the standard curve. Inhibitory concentration of 50% (IC₅₀) was determined by comparing average TNFa concentration to the positive control (THP-1 cells stimulated with opsonized zymosan). An average of n=4 replicate experiments was used to determine IC₅₀ values for Bay 11-7082 (see FIG. 13; IC₅₀=810 nM)) and rapamycin (IC₅₀=51 nM; FIG. 15). The IC₅₀ values for the following additional compounds were determined using this assay: IC₅₀ (nM): geldanamycin, 14; mycophenolic acid, 756; mofetil, 792; chlorpromazine, 6; CNI-1493, 0.15; SKF 86002, 831; 15-deoxy prostaglandin J2, 742; fascaplycin, 701; podophyllotoxin, 75; mithramycin, 570; daunorubicin, 195; celastrol, 87; chromomycin A3, 394; vinorelbine, 605; vinblastine, 65.

### Example 28

### Surgical Adhesions Model to Assess Fibrosis Inhibiting Agents

The rabbit uterine horn model is used to assess the anti-fibrotic capacity of formulations in vivo. Mature New Zealand White (NZW) female rabbits are placed under general anesthetic. Using aseptic precautions, the abdomen is opened in two layers at the midline to expose the uterus. Both uterine horns are lifted out of the abdominal cavity and assessed for size on the French Scale of catheters. Horns between #8 and #14 on the French Scale (2.5-4.5 mm diameter) are deemed suitable for this model. Both uterine horns and the opposing peritoneal wall are abraded with a #10 scalpel blade at a 45° angle over an area 2.5 cm in length and 0.4 cm in width until punctuate bleeding is observed. Abraded surfaces are tamponaded until bleeding stops. The individual horns are then opposed to the peritoneal wall and secured by two sutures placed 2 mm beyond the edges of the abraded area. The formulation is applied and the abdomen is closed in three layers. After 14 days, animals are evaluated post mortem with the extent and severity of adhesions being scored both quantitatively and qualitatively.

### Example 29

### Screening Assay for Assessing the Effect of Various Compounds on Cell Proliferation

Fibroblasts at 70-90% confluency were trypsinized, replated at 600 cells/well in media in 96-well plates and allowed to attach overnight. Mitoxantrone was prepared in DMSO at a concentration of 10⁻² M and diluted 10-fold to give a range of stock concentrations (10⁻⁸ M to 10⁻² M). Drug dilutions were diluted 1/1000 in media and added to cells to give a total volume of 200 μL/well. Each drug concentration was tested in triplicate wells. Plates containing fibroblasts and mitoxantrone were incubated at 37° C. for 72 hours (In vitro toxicol. (1990) 3: 219; Biotech. Histochem. (1993) 68: 29; Anal. Biochem. (1993) 213: 426).

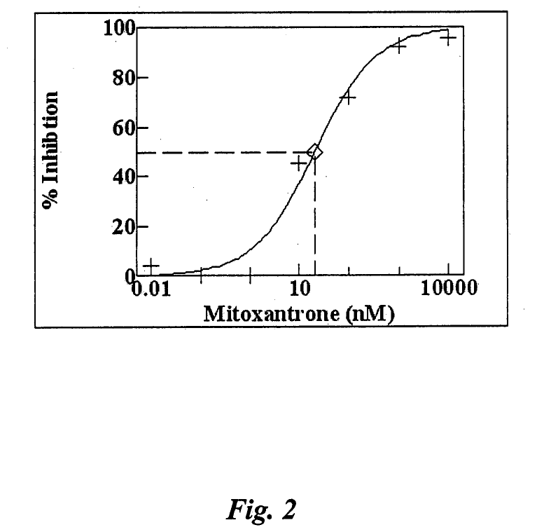

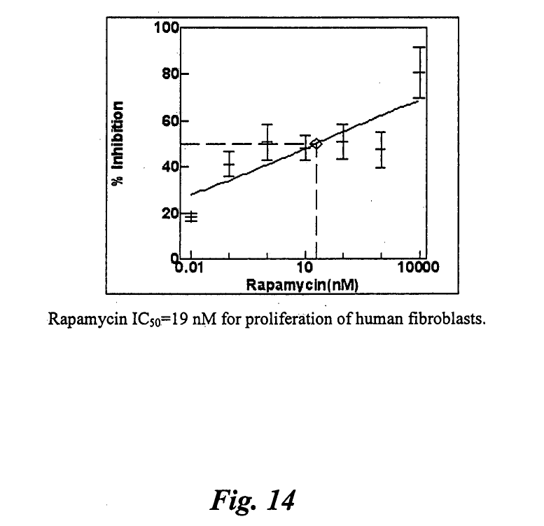

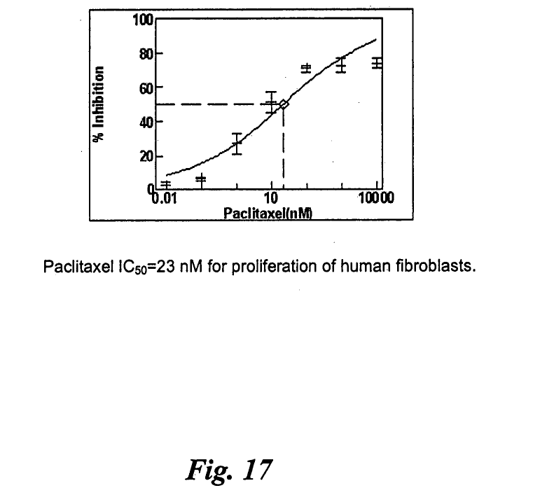

To terminate the assay, the media was removed by gentle aspiration. A 1/400 dilution of CYQUANT 400×GR dye indicator (Molecular Probes; Eugene, Oreg.) was added to 1× Cell Lysis buffer, and 200 μL of the mixture was added to the wells of the plate. Plates were incubated at room temperature, protected from light for 3-5 minutes. Fluorescence was read in a fluorescence microplate reader at ˜480 nm excitation wavelength and ˜520 nm emission maxima. Inhibitory concentration of 50% (IC₅₀) was determined by taking the average of triplicate wells and comparing average relative fluorescence units to the DMSO control. An average of n=4 replicate experiments was used to determine IC₅₀ values. The IC₅₀ values for the following compounds were determined using this assay: IC₅₀ (nM): paclitaxel, 23; mitoxantrone, 20; rapamycin, 19; mycophenolic Acid, 550; mofetil, 601; GW8510, 98; simvastatin, 885; doxorubicin, 84; geldanamycin, 11; anisomycin, 435; 17-AAG, 106; bleomycin, 86; halofuginone, 36; gemfibrozil, 164; ciprofibrate, 503; bezafibrate, 184; epirubicin hydrochloride, 57; topotemay, 81; fascaplysin, 854; tamoxifen, 13; etanidazole, 55; gemcitabine, 7; puromycin, 254; mithramycin, 156; daunorubicin, 51; L(−)-perillyl alcohol, 966; celastrol, 271; anacitabine, 225; oxalipatin, 380; chromomycin A3, 4; vinorelbine, 4; idarubicin, 34; nogalamycin, 5; 17-DMAG, 5; epothilone D, 2; vinblastine, 2; vincristine, 7; cytarabine, 137. The results of the assay for three of these compounds are shown in FIG. 2, FIG. 14, and FIG. 17.

### Example 30

### Evaluation of Paclitaxel Containing Mesh on Intimal Hyperplasia Development in a Rat Balloon Injury Carotid Artery Model

A rat balloon injury carotid artery model was used to demonstrate the efficacy of a paclitaxel containing mesh system on the development of intimal hyperplasia fourteen days following placement.

Control Group

Wistar rats weighing 400-500 g were anesthetized with 1.5% halothane in oxygen and the left external carotid artery was exposed. An A 2 French Fogarty balloon embolectomy catheter (Baxter, Irvine, Calif.) was advanced through an arteriotomy in the external carotid artery down the left common carotid artery to the aorta. The balloon was inflated with enough saline to generate slight resistance (approximately 0.02 ml) and it was withdrawn with a twisting motion to the carotid bifurcation. The balloon was then deflated and the procedure repeated twice more. This technique produced distension of the arterial wall and denudation of the endothelium. The external carotid artery was ligated after removal of the catheter. The right common carotid artery was not injured and was used as a control.

Local Perivascular Paclitaxel Treatment

Immediately after injury of the left common carotid artery, a 1 cm long distal segment of the artery was exposed and treated with a 1×1 cm paclitaxel-containing mesh. The wound was then closed the animals were kept for 14 days.

Histology and Immunohistochemistry

At the time of sacrifice, the animals were euthanized with carbon dioxide and pressure perfused at 100 mmHg with 10% phosphate buffered formaldehyde for 15 minutes. Both carotid arteries were harvested and left overnight in fixative. The fixed arteries were processed and embedded in paraffin wax. Serial cross-sections were cut at 3 μm thickness every 2 mm within and outside the implant region of the injured left carotid artery and at corresponding levels in the control right carotid artery. Cross-sections were stained with Mayer's hematoxylin-and-eosin for cell count and with Movat's pentachrome stains for morphometry analysis and for extracellular matrix composition assessment.

Results

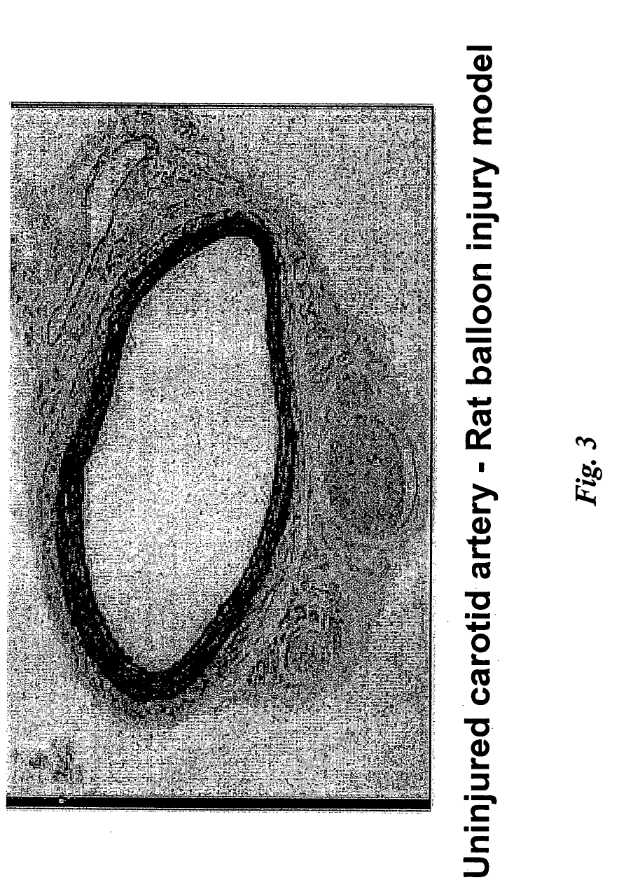

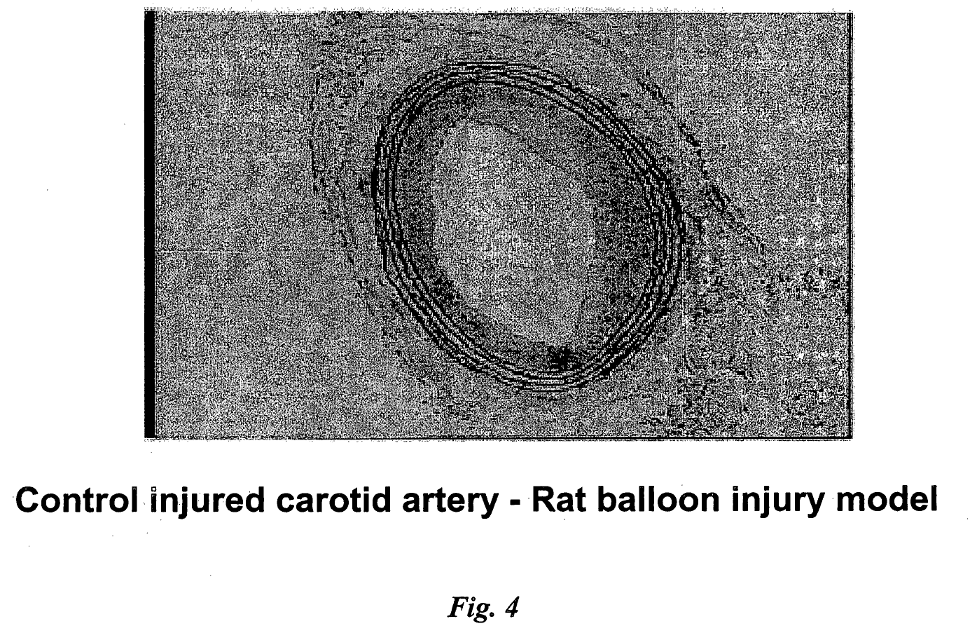

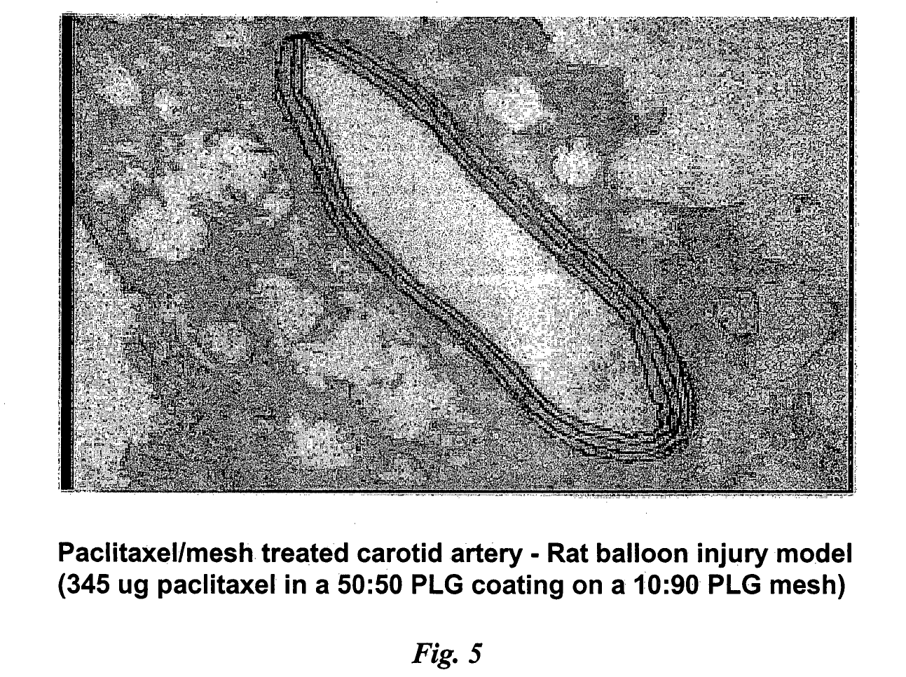

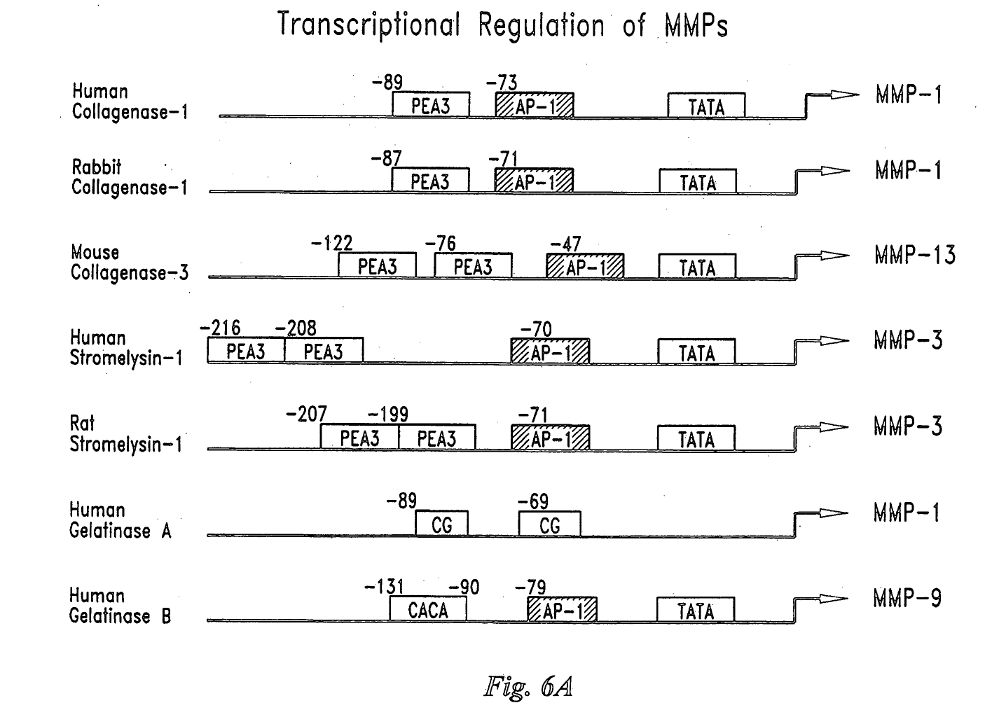

From FIGS. 3-5, it is evident that the perivascular delivery of paclitaxel using the paclitaxel mesh formulation resulted is a dramatic reduction in intimal hyperplasia.

### Example 31

### Effect of Paclitaxel and Other Anti-Microtubule Agents on Matrix Metalloproteinase Production

A. Materials and Methods

1. IL-1 Stimulated AP-1 Transcriptional Activity is Inhibited by Paclitaxel

Chondrocytes were transfected with constructs containing an AP-1 driven CAT reporter gene, and stimulated with IL-1, IL-1 (50 ng/ml) was added and incubated for 24 hours in the absence and presence of paclitaxel at various concentrations. Paclitaxel treatment decreased CAT activity in a concentration dependent manner (mean±SD). The data noted with an asterisk (*) have signifimayce compared with IL-1-induced CAT activity according to a t-test, P<0.05. The results shown are representative of three independent experiments.

2. Effect of Paclitaxel on IL-1 Induced AP-1 DNA Binding Activity, AP-1 DNA

Binding activity was assayed with a radiolabeled human AP-1 sequence probe and gel mobility shift assay. Extracts from chondrocytes untreated or treated with various amounts of paclitaxel (10⁻⁷ to 10⁻⁵ M) followed by IL-1β (20 ng/ml) were incubated with excess probe on ice for 30 minutes, followed by non-denaturing gel electrophoresis. The “com” lane contains excess unlabeled AP-1 oligonucleotide. The results shown are representative of three independent experiments.

3. Effect of Paclitaxel on IL-1 Induced MMP-1 and MMP-3 mRNA Expression

Cells were treated with paclitaxel at various concentrations (10⁻⁷ to 10⁻⁵ M) for 24 hours, then treated with IL-1β (20 ng/ml) for additional 18 hours in the presence of paclitaxel. Total RNA was isolated, and the MMP-1 mRNA levels were determined by Northern blot analysis. The blots were subsequently stripped and reprobed with ³²P-radiolabeled rat GAPDH cDNA, which was used as a housekeeping gene. The results shown are representative of four independent experiments. Quantitation of collagenase-1 and stromelysin-expression mRNA levels. The MMP-1 and MMP-3 expression levels were normalized with GAPDH.

4. Effect of Other Anti-Microtubules on Collagenase Expression

Primary chondrocyte cultures were freshly isolated from calf cartilage. The cells were plated at 2.5×10⁶ per ml in 100×20 mm culture dishes and incubated in Ham's F12 medium containing 5% FBS overnight at 37° C. The cells were starved in serum-free medium overnight and then treated with anti-microtubule agents at various concentrations for 6 hours. IL-1 (20 ng/ml) was then added to each plate and the plates incubated for an additional 18 hours. Total RNA was isolated by the acidified guanidine isothiocyanate method and subjected to electrophoresis on a denatured gel. Denatured RNA samples (15 μg) were analyzed by gel electrophoresis in a 1% denatured gel, transferred to a nylon membrane and hydridized with the ³²P-labeled collagenase cDNA probe. ³²P-labeled glyceraldehyde phosphate dehydrase (GAPDH) cDNA as an internal standard to ensure roughly equal loading. The exposed films were smayned and quantitatively analyzed with IMAGEQUANT.

B. Results

1. Promoters on the Family of Matrix Metalloproteinases

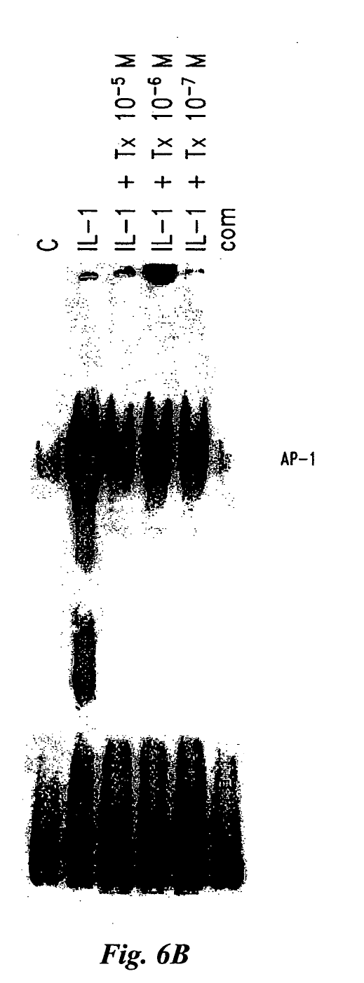

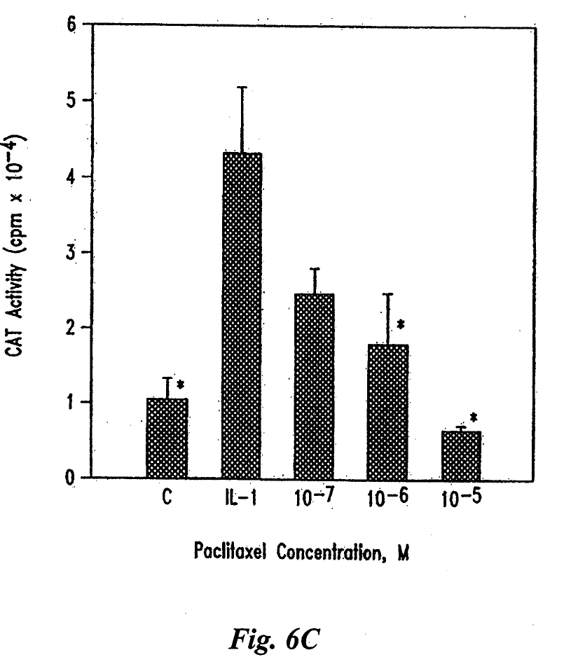

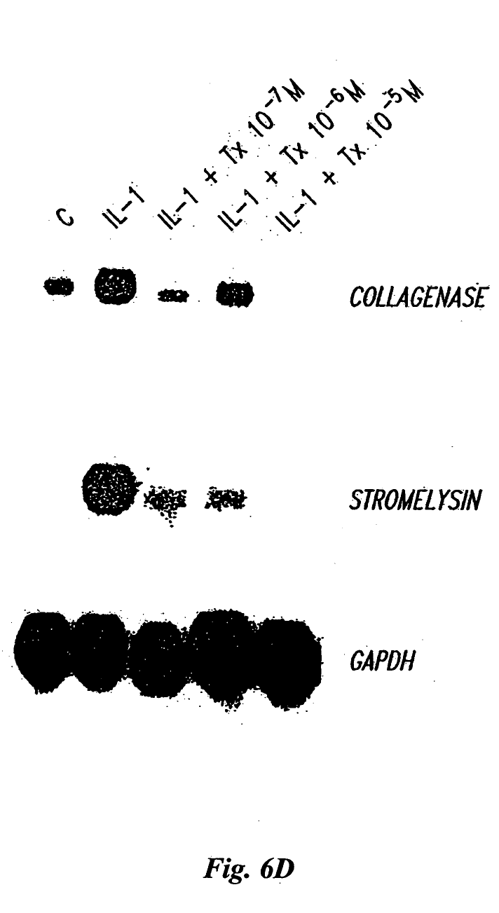

FIG. 6A shows that all matrix metalloproteinases contained the transcriptional elements AP-1 and PEA-3 with the exception of Gelatinase B. It has been well established that expression of matrix metalloproteinases such as collagenases and stromelysins are dependent on the activation of the transcription factors AP-1. Thus inhibitors of AP-1 may inhibit the expression of matrix metalloproteinases.

2. Effect of Paclitaxel on AP-1 Transcriptional Activity

As demonstrated in FIG. 6B, IL-1 stimulated AP-1 transcriptional activity 5-fold. Pretreatment of transiently transfected chondrocytes with paclitaxel reduced IL-1 induced AP-1 reporter gene CAT activity. Thus, IL-1 induced AP-1 activity was reduced in chondrocytes by paclitaxel in a concentration dependent manner (10⁻⁷ to 10⁻⁵ M). These data demonstrated that paclitaxel was a potent inhibitor of AP-1 activity in chondrocytes.

3. Effect of Paclitaxel on AP-1 DNA Binding Activity

To confirm that paclitaxel inhibition of AP-1 activity was not due to nonspecific effects, the effect of paclitaxel on IL-1 induced AP-1 binding to oligonucleotides using chondrocyte nuclear lysates was examined. As shown in FIG. 6C, IL-1 induced binding activity decreased in lysates from chondrocyte which had been pretreated with paclitaxel at concentration 10⁻⁷ to 10⁻⁵ M for 24 hours. Paclitaxel inhibition of AP-1 transcriptional activity closely correlated with the decrease in AP-1 binding to DNA.

4. Effect of Paclitaxel on Collagenase and Stromelysin Expression

Since paclitaxel was a potent inhibitor of AP-1 activity, the effect of paclitaxel or IL-1 induced collagenase and stromelysin expression, two important matrix metalloproteinases involved in inflammatory diseases was examined. Briefly, as shown in FIG. 6D, IL-1 induction increases collagenase and stromelysin mRNA levels in chondrocytes. Pretreatment of chondrocytes with paclitaxel for 24 hours signifimaytly reduced the levels of collagenase and stromelysin mRNA. At 10⁻⁵ M paclitaxel, there was complete inhibition. The results show that paclitaxel completely inhibited the expression of two matrix metalloproteinases at concentrations similar to which it inhibits AP-1 activity.

5. Effect of Other Anti-Microtubules on Collagenase Expression

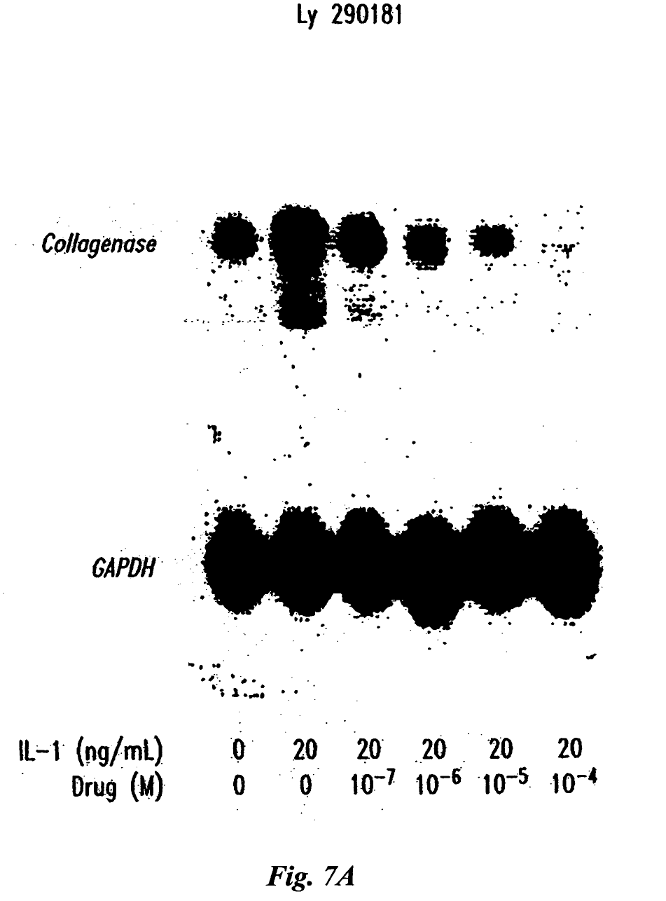

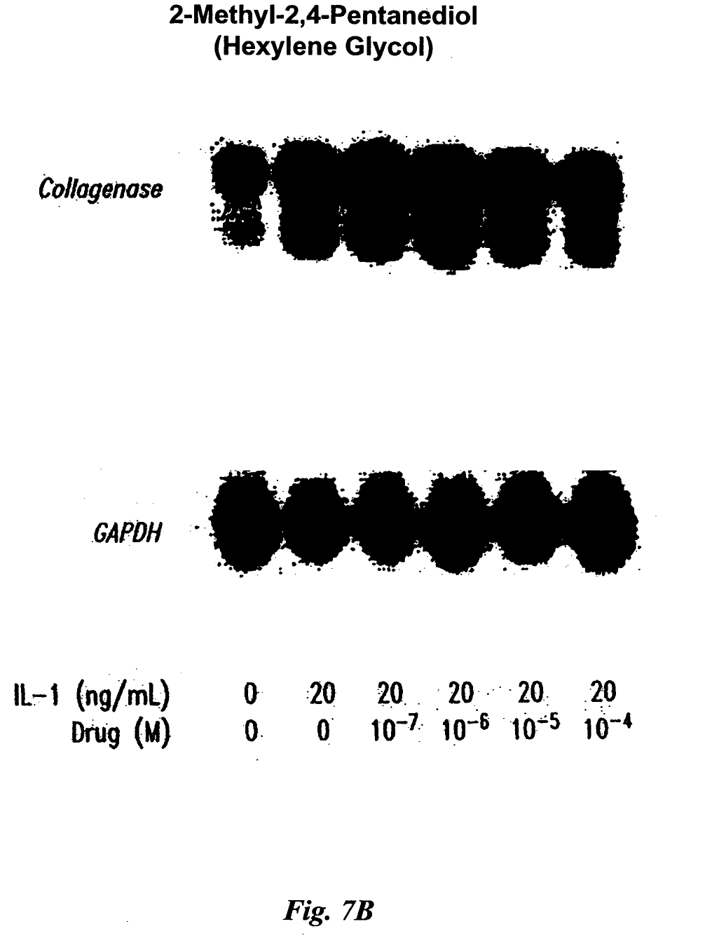

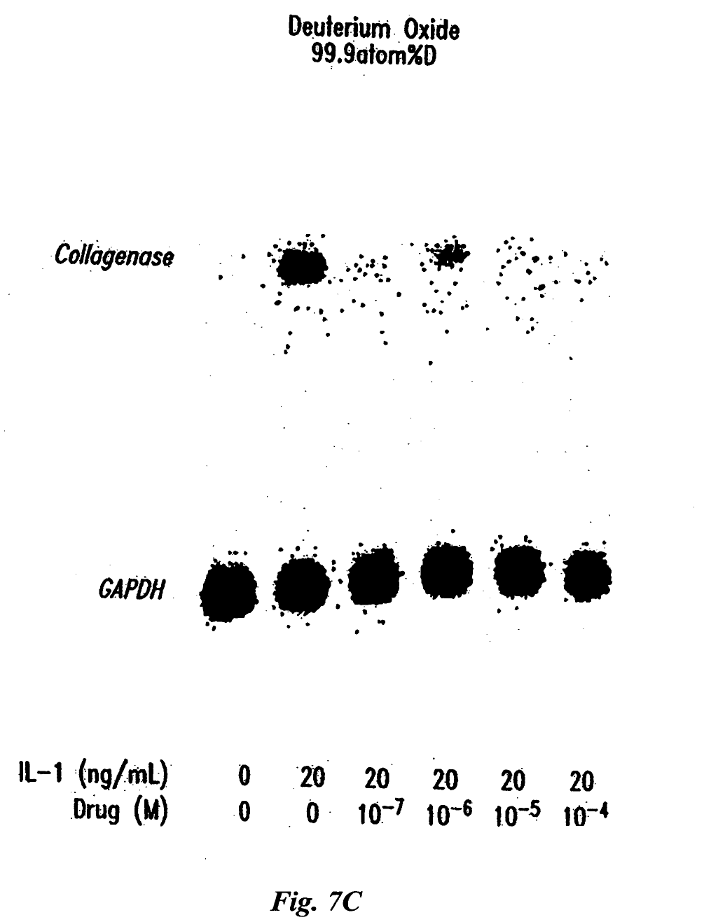

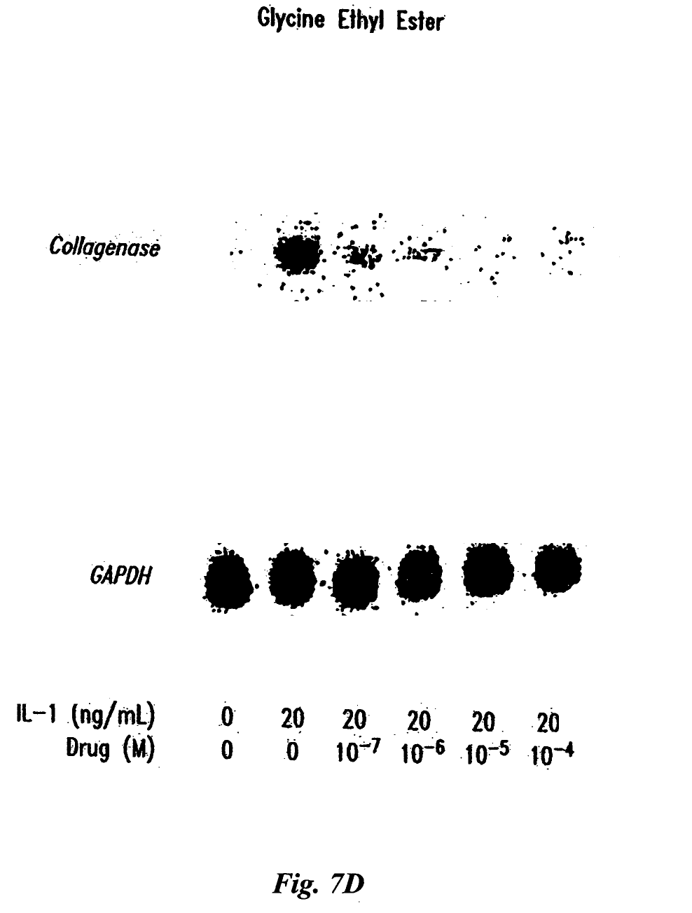

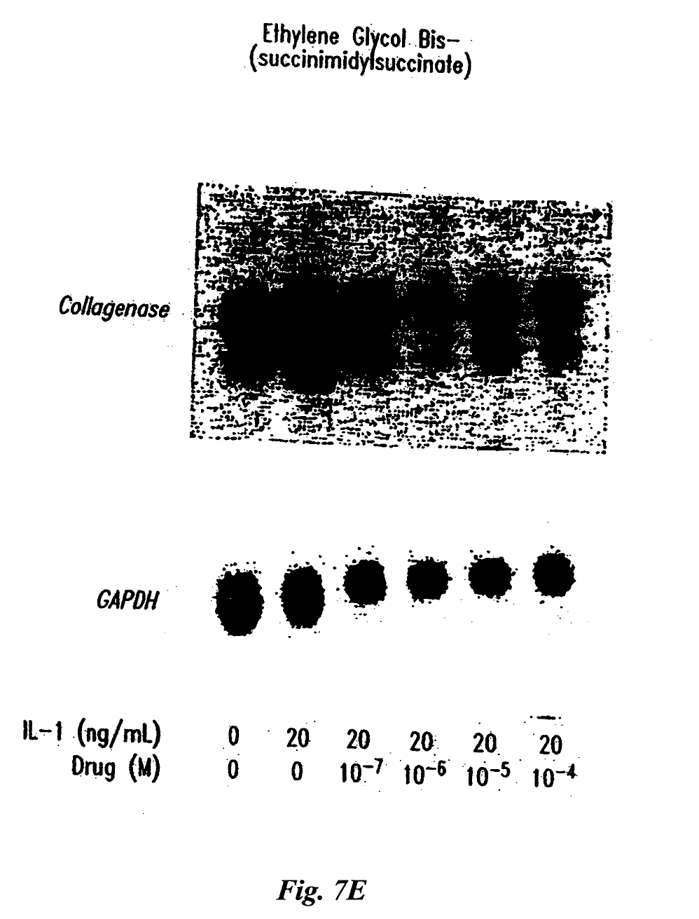

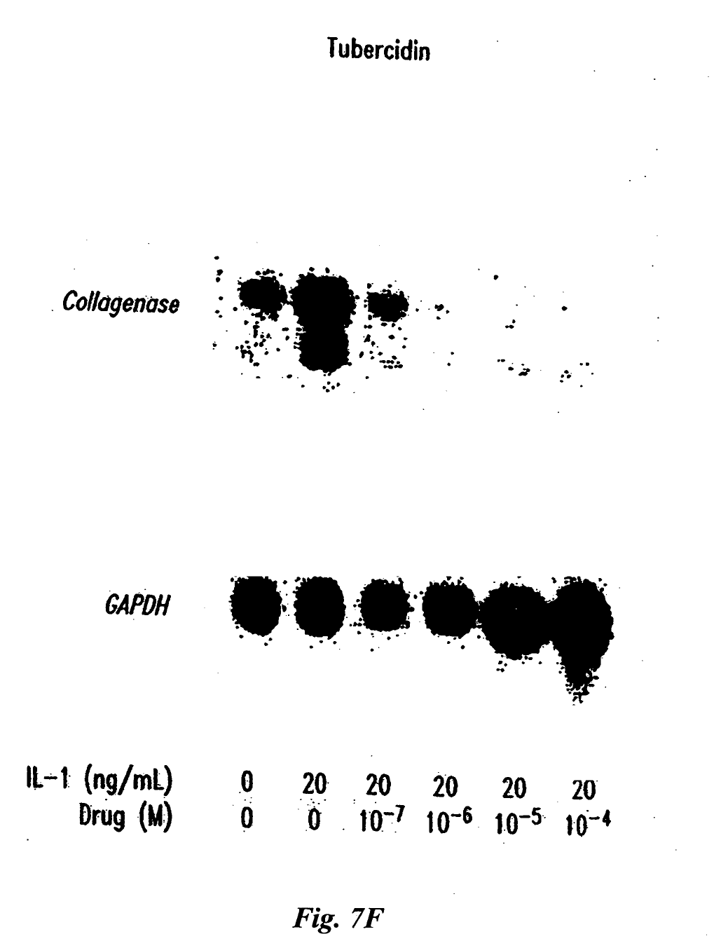

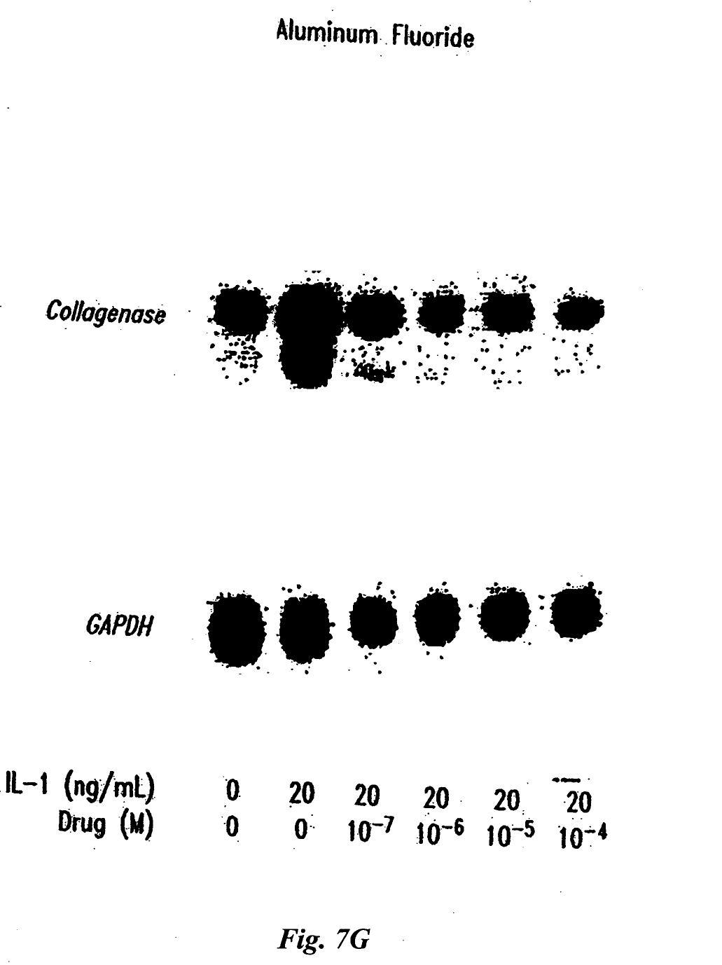

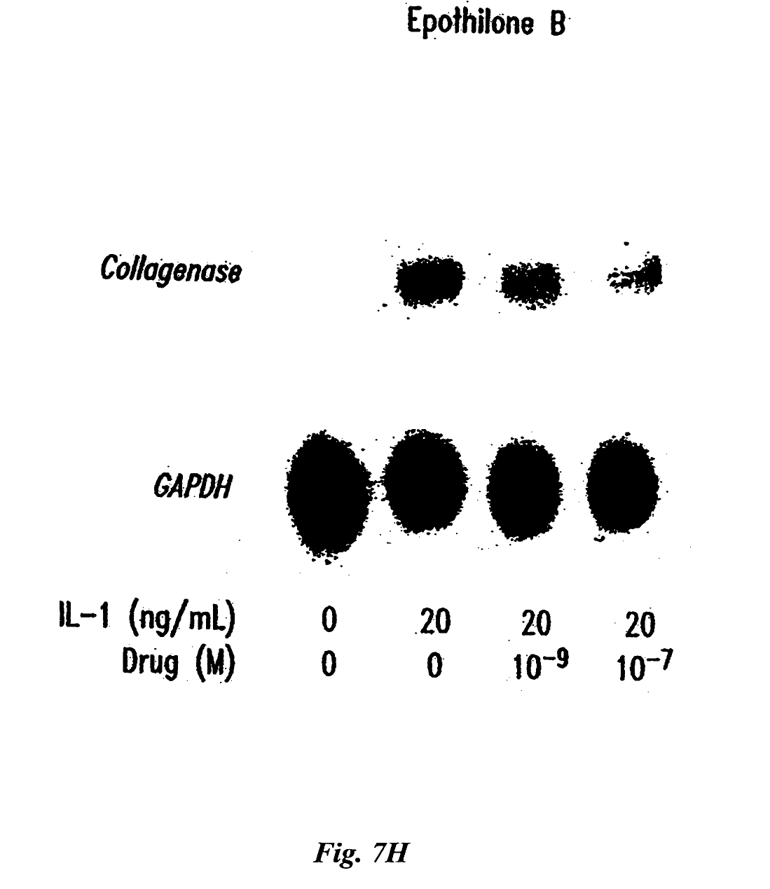

FIGS. 7A-H demonstrate that anti-microtubule agents inhibited collagenase expression. Expression of collagenase was stimulated by the addition of IL-1 which is a proinflammatory cytokine. Pre-incubation of chondrocytes with various anti-microtubule agents, specifically LY290181, hexylene glycol, deuterium oxide, glycine ethyl ester, ethylene glycol bis-(succinimidylsuccinate), tubercidin, AIF₃, and epothilone, all prevented IL-1-induced collagenase expression at concentrations as low as 1×10⁻⁷ M.

C. Discussion

Paclitaxel was capable of inhibiting collagenase and stromelysin expression in vitro at concentrations of 10⁻⁶ M. Since this inhibition may be explained by the inhibition of AP-1 activity, a required step in the induction of all matrix metalloproteinases with the exception of gelatinase B, it is expected that paclitaxel may inhibit other matrix metalloproteinases which are AP-1 dependent. The levels of these matrix metalloproteinases are elevated in all inflammatory diseases and play a principle role in matrix degradation, cellular migration and proliferation, and angiogenesis. Thus, paclitaxel inhibition of expression of matrix metalloproteinases such as collagenase and stromelysin will have a beneficial effect in inflammatory diseases.

In addition to paclitaxel's inhibitory effect on collagenase expression, LY290181, hexylene glycol, deuterium oxide, glycine ethyl ester, AIF₃, tubercidin epothilone, and ethylene glycol bis-(succinimidylsuccinate), all prevented IL-1-induced collagenase expression at concentrations as low as 1×10⁻⁷ M. Thus, anti-microtubule agents are capable of inhibiting the AP-1 pathway at varying concentrations.

### Example 32

### Inhibition of Angiogenesis by Paclitaxel

A. Chick Chorioallantoic Membrane (“CAM”) Assays

Fertilized, domestic chick embryos were incubated for 3 days prior to shell-less culturing. In this procedure, the egg contents were emptied by removing the shell located around the air space. The interior shell membrane was then severed and the opposite end of the shell was perforated to allow the contents of the egg to gently slide out from the blunted end. The egg contents were emptied into round-bottom sterilized glass bowls and covered with petri dish covers. These were then placed into an incubator at 90% relative humidity and 3% CO₂ and incubated for 3 days.

Paclitaxel (Sigma, St. Louis, Mich.) was mixed at concentrations of 0.25, 0.5, 1, 5, 10, 30 μg per 10 ul aliquot of 0.5% aqueous methylcellulose. Since paclitaxel is insoluble in water, glass beads were used to produce fine particles. Ten microliter aliquots of this solution were dried on parafilm for 1 hour forming disks 2 mm in diameter. The dried disks containing paclitaxel were then carefully placed at the growing edge of each CAM at day 6 of incubation. Controls were obtained by placing paclitaxel-free methylcellulose disks on the CAMs over the same time course. After a 2 day exposure (day 8 of incubation) the vasculature was examined with the aid of a stereomicroscope. Liposyn II, a white opaque solution, was injected into the CAM to increase the visibility of the vascular details. The vasculature of unstained, living embryos were imaged using a Zeiss stereomicroscope which was interfaced with a video camera (Dage-MTI Inc., Michigan City, Ind.). These video signals were then displayed at 160× magnification and captured using an image analysis system (Vidas, Kontron; Etching, Germany). Image negatives were then made on a graphics recorder (Model 3000; Matrix Instruments, Orangeburg, N.Y.).

The membranes of the 8 day-old shell-less embryo were flooded with 2% glutaraldehyde in 0.1M sodium cacodylate buffer; additional fixative was injected under the CAM. After 10 minutes in situ, the CAM was removed and placed into fresh fixative for 2 hours at room temperature. The tissue was then washed overnight in cacodylate buffer containing 6% sucrose. The areas of interest were postfixed in 1% osmium tetroxide for 1.5 hours at 4° C. The tissues were then dehydrated in a graded series of ethanols, solvent exchanged with propylene oxide, and embedded in Spurr resin. Thin sections were cut with a diamond knife, placed on copper grids, stained, and examined in a Joel 1200EX electron microscope. Similarly, 0.5 mm sections were cut and stained with toluene blue for light microscopy.

At day 11 of development, chick embryos were used for the corrosion casting technique. Mercox resin (Ted Pella, Inc., Redding, Calif.) was injected into the CAM vasculature using a 30-gauge hypodermic needle. The casting material consisted of 2.5 grams of Mercox CL-2B polymer and 0.05 grams of catalyst (55% benzoyl peroxide) having a 5 minute polymerization time. After injection, the plastic was allowed to sit in situ for an hour at room temperature and then overnight in an oven at 65° C. The CAM was then placed in 50% aqueous solution of sodium hydroxide to digest all organic components. The plastic casts were washed extensively in distilled water, air-dried, coated with gold/palladium, and viewed with the Philips 501B smayning electron microscope.

Results of the assay were as follows. At day 6 of incubation, the embryo was centrally positioned to a radially expanding network of blood vessels; the CAM developed adjacent to the embryo. These growing vessels lie close to the surface and are readily visible making this system an idealized model for the study of angiogenesis. Living, unstained capillary networks of the CAM may be imaged noninvasively with a stereomicroscope.

Transverse sections through the CAM show an outer ectoderm consisting of a double cell layer, a broader mesodermal layer containing capillaries which lie subjacent to the ectoderm, adventitial cells, and an inner, single endodermal cell layer. At the electron microscopic level, the typical structural details of the CAM capillaries are demonstrated. Typically, these vessels lie in close association with the inner cell layer of ectoderm.

After 48 hours exposure to paclitaxel at concentrations of 0.25, 0.5, 1, 5, 10, or 30 μg, each CAM was examined under living conditions with a stereomicroscope equipped with a video/computer interface in order to evaluate the effects on angiogenesis. This imaging setup was used at a magnification of 160× which permitted the direct visualization of blood cells within the capillaries; thereby blood flow in areas of interest may be easily assessed and recorded. For this study, the inhibition of angiogenesis was defined as an area of the CAM (measuring 2-6 mm in diameter) lacking a capillary network and vascular blood flow. Throughout the experiments, avascular zones were assessed on a 4 point avascular gradient (Table 1). This scale represents the degree of overall inhibition with maximal inhibition represented as a 3 on the avascular gradient scale. Paclitaxel was very consistent and induced a maximal avascular zone (6 mm in diameter or a 3 on the avascular gradient scale) within 48 hours depending on its concentration.

TABLE 1
Avascular Gradient

0 - normal vascularity
1 - lacking some microvascular movement
2* - small avascular zone approximately 2 mm in diameter
3* - avascularity extending beyond the disk (6 mm in diameter)
*indicates a positive antiangiogenesis response

The dose-dependent, experimental data of the effects of paclitaxel at different concentrations are shown in Table 2.

TABLE 2

| Agent | Delivery Vehicle | Concentration | Inhibition/n |
|---|---|---|---|
| paclitaxel | methylcellulose (10 ul) | 0.25 ug | 2/11 |
| | methylcellulose (10 ul) | 0.5 ug | 6/11 |
| | methylcellulose (10 ul) | 1 ug | 6/15 |
| | methylcellulose (10 ul) | 5 ug | 20/27 |
| | methylcellulose (10 ul) | 10 ug | 16/21 |
| | methylcellulose (10 ul) | 30 ug | 31/31 |

Typical paclitaxel-treated CAMs are also shown with the transparent methylcellulose disk centrally positioned over the avascular zone measuring 6 mm in diameter. At a slightly higher magnification, the periphery of such avascular zones is clearly evident; the surrounding functional vessels were often redirected away from the source of paclitaxel. Such angular redirecting of blood flow was never observed under normal conditions. Another feature of the effects of paclitaxel was the formation of blood islands within the avascular zone representing the aggregation of blood cells.

In summary, this study demonstrated that 48 hours after paclitaxel application to the CAM, angiogenesis was inhibited. The blood vessel inhibition formed an avascular zone which was represented by three transitional phases of paclitaxel's effect. The central, most affected area of the avascular zone contained disrupted capillaries with extravasated red blood cells; this indicated that intercellular junctions between endothelial cells were absent. The cells of the endoderm and ectoderm maintained their intercellular junctions and therefore these germ layers remained intact; however, they were slightly thickened. As the normal vascular area was approached, the blood vessels retained their junctional complexes and therefore also remained intact. At the periphery of the paclitaxel-treated zone, further blood vessel growth was inhibited which was evident by the typical redirecting or “elbowing” effect of the blood vessels.

### Example 33

### Screening Assay for Assessing the Effect of Paclitaxel on Smooth Muscle Cell Migration

Primary human smooth muscle cells were starved of serum in smooth muscle cell basal media containing insulin and human basic fibroblast growth factor (bFGF) for 16 hours prior to the assay. For the migration assay, cells were trypsinized to remove cells from flasks, washed with migration media and diluted to a concentration of 2-2.5×10⁵ cells/mL in migration media. Migration media consists of phenol red free Dulbecco's Modified Eagle Medium (DMEM) containing 0.35% human serum albumin. A 100 μL volume of smooth muscle cells (approximately 20,000-25,000 cells) was added to the top of a Boyden chamber assembly (Chemicon QCM CHEMOTAXIS 96-well migration plate). To the bottom wells, the chemotactic agent, recombinant human platelet derived growth factor (rhPDGF-BB) was added at a concentration of 10 ng/mL in a total volume of 150 μL. Paclitaxel was prepared in DMSO at a concentration of 10⁻² M and serially diluted 10-fold to give a range of stock concentrations (10⁻⁸ M to 10⁻² M). Paclitaxel was added to cells by directly adding paclitaxel DMSO stock solutions, prepared earlier, at a 1/1000 dilution, to the cells in the top chamber. Plates were incubated for 4 hours to allow cell migration.

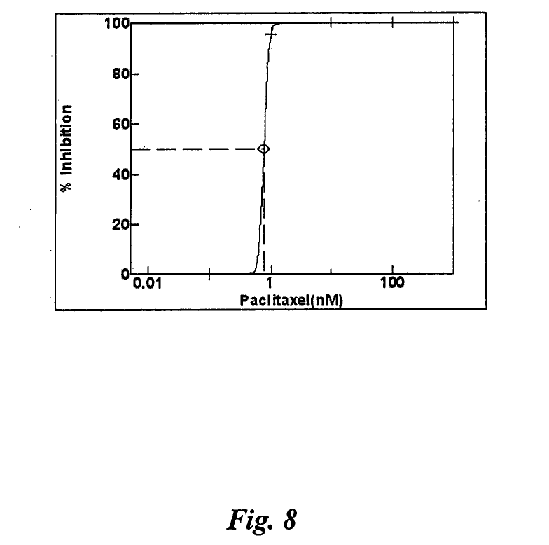

At the end of the 4 hour period, cells in the top chamber were discarded and the smooth muscle cells attached to the underside of the filter were detached for 30 minutes at 37° C. in Cell Detachment Solution (Chemicon). Dislodged cells were lysed in lysis buffer containing the DNA binding CYQUANT GR dye and incubated at room temperature for 15 minutes. Fluorescence was read in a fluorescence microplate reader at ˜480 nm excitation wavelength and 520 nm emission maxima. Relative fluorescence units from triplicate wells were averaged after subtracting background fluorescence (control chamber without chemoattractant) and average number of cells migrating was obtained from a standard curve of smooth muscle cells serially diluted from 25,000 cells/well down to 98 cells/well. Inhibitory concentration of 50% (IC₅₀) was determined by comparing the average number of cells migrating in the presence of paclitaxel to the positive control (smooth muscle cell chemotaxis in response to rhPDGF-BB). See FIG. 8 (IC₅₀=0.76 nM). References: Biotechniques (2000) 29: 81; J. Immunol Methods (2001) 254: 85.

### Example 34

### Screening Assay for Assessing the Effect of Various Compounds on Il-1β Production by Macrophages

The human macrophage cell line, THP-1 was plated in a 12 well plate such that each well contains 1×10⁶ cells in 2 mL of media containing 10% FCS. Opsonized zymosan was prepared by resuspending 20 mg of zymosan A in 2 mL of ddH₂O and homogenizing until a uniform suspension was obtained. Homogenized zymosan was pelleted at 250 g and resuspended in 4 mL of human serum for a final concentration of 5 mg/mL and incubated in a 37° C. water bath for 20 minutes to enable opsonization. Geldanamycin was prepared in DMSO at a concentration of 10⁻² M and serially diluted 10-fold to give a range of stock concentrations (10⁻⁸ M to 10⁻² M).

THP-1 cells were stimulated to produce IL-1β by the addition of 1 mg/mL opsonized zymosan. Geldanamycin was added to THP-1 cells by directly adding DMSO stock solutions, prepared earlier, at a 1/1000 dilution, to each well. Each drug concentration was tested in triplicate wells. Plates were incubated at 37° C. for 24 hours.

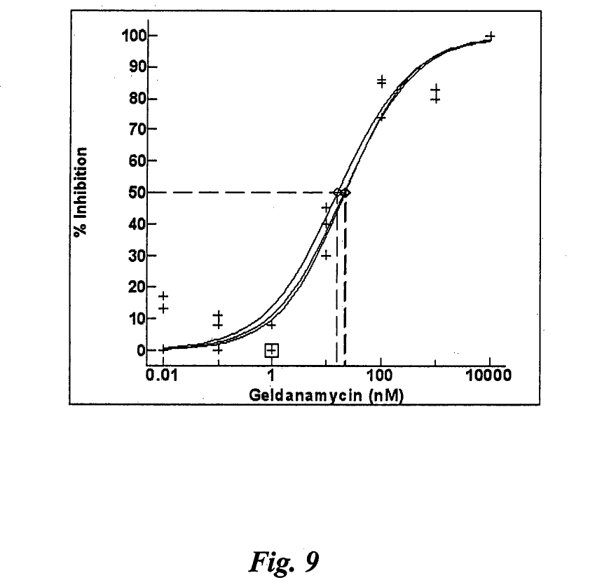

After a 24 hour stimulation, supernatants were collected to quantify IL-1β production. IL-1β concentrations in the supernatants were determined by ELISA using recombinant human IL-1β to obtain a standard curve. A 96-well MaxiSorb plate was coated with 100 μL of anti-human IL-1β Capture Antibody diluted in Coating Buffer (0.1M Sodium carbonate pH 9.5) overnight at 4° C. The dilution of Capture Antibody used was lot-specific and was determined empirically. Capture antibody was then aspirated and the plate washed 3 times with Wash Buffer (PBS, 0.05% TWEEN-20). Plates were blocked for 1 hour at room temperature with 200 μL/well of Assay Diluent (PBS, 10% FCS pH 7.0). After blocking, plates were washed 3 times with Wash Buffer. Standards and sample dilutions were prepared as follows: (a) sample supernatants were diluted 1/4 and ?; (b) recombinant human IL-1β was prepared at 1000 pg/mL and serially diluted to yield as standard curve of 15.6 μg/mL to 1000 μg/mL. Sample supernatants and standards were assayed in triplicate and were incubated at room temperature for 2 hours after addition to the plate coated with Capture Antibody. The plates were washed 5 times and incubated with 100 μL of Working Detector (biotinylated anti-human IL-1β detection antibody+avidin-HRP) for 1 hour at room temperature. Following this incubation, the plates were washed 7 times and 100 μL of Substrate Solution (Tetramethylbenzidine, H₂O₂) was added to plates and incubated for 30 minutes at room temperature. Stop Solution (2 N H₂SO₄) was then added to the wells and a yellow color reaction was read at 450 nm with ? correction at 570 nm. Mean absorbance was determined from triplicate data readings and the mean background was subtracted. IL-1β concentration values were obtained from the standard curve. Inhibitory concentration of 50% (IC₅₀) was determined by comparing average IL-1β concentration to the positive control (THP-1 cells stimulated with opsonized zymosan). An average of n=4 replicate experiments was used to determine IC₅₀ values for geldanamycin (IC₅₀=20 nM). See FIG. 9. The IC₅₀ values for the following additional compounds were determined using this assay: IC₅₀ (nM): mycophenolic acid 2888 nM); anisomycin, 127; rapamycin, 0.48; halofuginone, 919; IDN-6556, 642; epirubicin hydrochloride, 774; topotemay, 509; fascaplycin, 425; daunorubicin, 517; celastrol, 23; oxalipatin, 107; chromomycin A3, 148.

References: J. Immunol. (2000) 165: 411-418; J. Immunol. (2000) 164: 4804-4811; J. Immunol Meth. (2000) 235 (1-2): 33-40.

### Example 35

### Screening Assay for Assessing the Effect of Various Compounds on IL-8 Production by Macrophages

The human macrophage cell line, THP-1 was plated in a 12 well plate such that each well contains 1×10⁶ cells in 2 mL of media containing 10% FCS. Opsonized zymosan was prepared by resuspending 20 mg of zymosan A in 2 mL of ddH₂O and homogenizing until a uniform suspension was obtained. Homogenized zymosan was pelleted at 250 g, resuspended in 4 mL of human serum for a final concentration of 5 mg/mL, and incubated in a 37° C. water bath for 20 minutes to enable opsonization. Geldanamycin was prepared in DMSO at a concentration of 10⁻² M and serially diluted 10-fold to give a range of stock concentrations (10⁻⁸ M to 10⁻² M).

THP-1 cells were stimulated to produce IL-8 by the addition of 1 mg/mL opsonized zymosan. Geldanamycin was added to THP-1 cells by directly adding DMSO stock solutions, prepared earlier, at a 1/1000 dilution, to each well. Each drug concentration was tested in triplicate wells. Plates were incubated at 37° C. for 24 hours.

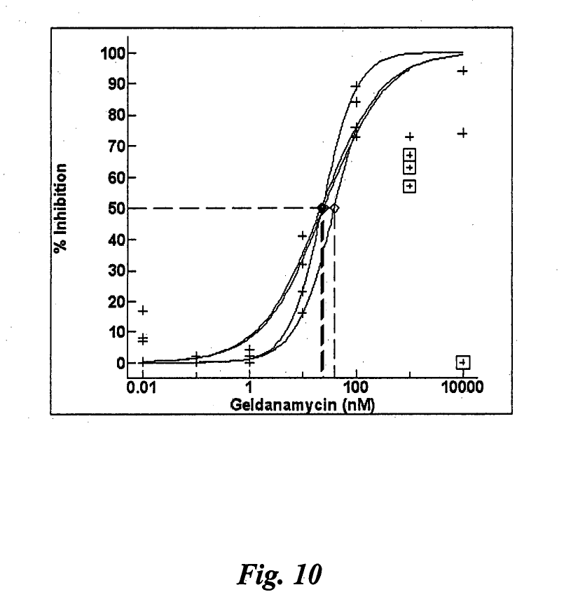

After a 24 hour stimulation, supernatants were collected to quantify IL-8 production. IL-8 concentrations in the supernatants were determined by ELISA using recombinant human IL-8 to obtain a standard curve. A 96-well MAXISORB plate was coated with 100 μL of anti-human IL-8 Capture Antibody diluted in Coating Buffer (0.1M sodium carbonate pH 9.5) overnight at 4° C. The dilution of Capture Antibody used was lot-specific and was determined empirically. Capture antibody was then aspirated and the plate washed 3 times with Wash Buffer (PBS, 0.05% TWEEN-20). Plates were blocked for 1 hour at room temperature with 200 μL/well of Assay Diluent (PBS, 10% FCS pH 7.0). After blocking, plates were washed 3 times with Wash Buffer. Standards and sample dilutions were prepared as follows: (a) sample supernatants were diluted 1/100 and 1/1000; (b) recombinant human IL-8 was prepared at 200 pg/mL and serially diluted to yield as standard curve of 3.1 pg/mL to 200 pg/mL. Sample supernatants and standards were assayed in triplicate and were incubated at room temperature for 2 hours after addition to the plate coated with Capture Antibody. The plates were washed 5 times and incubated with 100 μL of Working Detector (biotinylated anti-human IL-8 detection antibody+avidin-HRP) for 1 hour at room temperature. Following this incubation, the plates were washed 7 times and 100 μL of Substrate Solution (Tetramethylbenzidine, H₂O₂) was added to plates and incubated for 30 minutes at room temperature. Stop Solution (2 N H₂SO₄) was then added to the wells and a yellow color reaction was read at 450 nm with ? correction at 570 nm. Mean absorbance was determined from triplicate data readings and the mean background was subtracted. IL-8 concentration values were obtained from the standard curve. Inhibitory concentration of 50% (IC₅₀) was determined by comparing average IL-8 concentration to the positive control (THP-1 cells stimulated with opsonized zymosan). An average of n=4 replicate experiments was used to determine IC₅₀ values for geldanamycin (IC₅₀=27 nM). See FIG. 10. The IC₅₀ values for the following additional compounds were determined using this assay: IC₅₀ (nM): 17-AAG, 56; mycophenolic acid, 549; resveratrol, 507; rapamycin, 4; 41; SP600125, 344; halofuginone, 641; D-mannose-6-phosphate, 220; epirubicin hydrochloride, 654; topotemay, 257; mithramycin, 33; daunorubicin, 421; celastrol, 490; chromomycin A3, 36.

References: J. Immunol. (2000) 165: 411-418; J. Immunol. (2000) 164: 4804-4811; J. Immunol Meth. (2000) 235 (1-2): 33-40.

### Example 36

### Screening Assay for Assessing the Effect of Various Compounds on MCP-1 Production by Macrophages

The human macrophage cell line, THP-1 was plated in a 12 well plate such that each well contains 1×10⁶ cells in 2 mL of media containing 10% FCS. Opsonized zymosan was prepared by resuspending 20 mg of zymosan A in 2 mL of ddH₂O and homogenizing until a uniform suspension was obtained. Homogenized zymosan was pelleted at 250 g and resuspended in 4 mL of human serum for a final concentration of 5 mg/mL and incubated in a 37° C. water bath for 20 minutes to enable opsonization. Geldanamycin was prepared in DMSO at a concentration of 10⁻² M and serially diluted 10-fold to give a range of stock concentrations (10⁻⁸ M to 10⁻² M).

THP-1 cells were stimulated to produce MCP-1 by the addition of 1 mg/mL opsonized zymosan. Eldanamycin was added to THP-1 cells by directly adding DMSO stock solutions, prepared earlier, at a 1/1000 dilution, to each well. Each drug concentration was tested in triplicate wells. Plates were incubated at 37° C. for 24 hours.

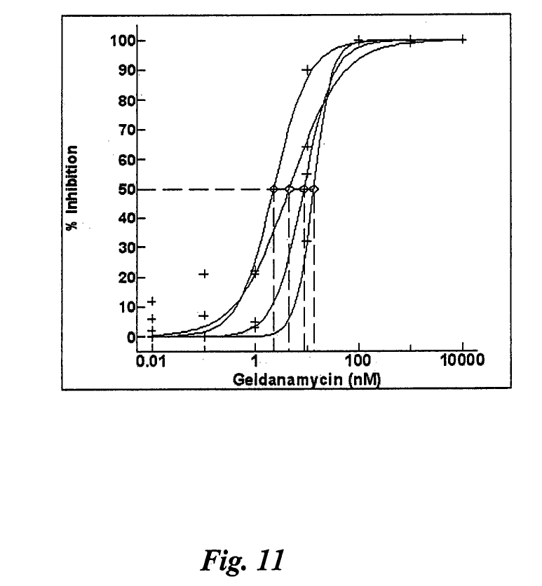

After a 24 hour stimulation, supernatants were collected to quantify MCP-1 production. MCP-1 concentrations in the supernatants were determined by ELISA using recombinant human MCP-1 to obtain a standard curve. A 96-well MaxiSorb plate was coated with 100 μL of anti-human MCP-1 Capture Antibody diluted in Coating Buffer (0.1M Sodium carbonate pH 9.5) overnight at 4° C. The dilution of Capture Antibody used was lot-specific and was determined empirically. Capture antibody was then aspirated and the plate washed 3 times with Wash Buffer (PBS, 0.05% TWEEN-20). Plates were blocked for 1 hour at room temperature with 200 μL/well of Assay Diluent (PBS, 10% FCS pH 7.0). After blocking, plates were washed 3 times with Wash Buffer. Standards and sample dilutions were prepared as follows: (a) sample supernatants were diluted 1/100 and 1/1000; (b) recombinant human MCP-1 was prepared at 500 pg/mL and serially diluted to yield as standard curve of 7.8 pg/mL to 500 pg/mL. Sample supernatants and standards were assayed in triplicate and were incubated at room temperature for 2 hours after addition to the plate coated with Capture Antibody. The plates were washed 5 times and incubated with 100 μL of Working Detector (biotinylated anti-human MCP-1 detection antibody+avidin-HRP) for 1 hour at room temperature. Following this incubation, the plates were washed 7 times and 100 μL of Substrate Solution (tetramethylbenzidine, H₂O₂) was added to plates and incubated for 30 minutes at room temperature. Stop Solution (2 N H₂SO₄) was then added to the wells and a yellow color reaction was read at 450 nm with ? correction at 570 nm. Mean absorbance was determined from triplicate data readings and the mean background was subtracted. MCP-1 concentration values were obtained from the standard curve. Inhibitory concentration of 50% (IC₅₀) was determined by comparing average MCP-1 concentration to the positive control (THP-1 cells stimulated with opsonized zymosan). An average of n=4 replicate experiments was used to determine IC₅₀ values for geldanamycin (IC₅₀=7 nM). See FIG. 11. The IC₅₀ values for the following additional compounds were determined using this assay: IC₅₀ (nM): 17-AAG, 135; anisomycin, 71; mycophenolic acid, 764; mofetil, 217; mitoxantrone, 62; chlorpromazine, 0.011; 1-a-25 dihydroxy vitamin D₃, 1; Bay 58-2667, 216; 15-deoxy prostaglandin J2, 724; rapamycin, 0.05; CNI-1493, 0.02; BXT-51072, 683; halofuginone, 9; CYC 202, 306; topotemay, 514; fascaplycin, 215; podophyllotoxin, 28; gemcitabine, 50; puromycin, 161; mithramycin, 18; daunorubicin, 570; celastrol, 421; chromomycin A3, 37; vinorelbine, 69; tubercidin, 56; vinblastine, 19; vincristine, 16.

References: J. Immunol. (2000) 165: 411-418; J. Immunol. (2000) 164: 4804-4811; J. Immunol Meth. (2000) 235 (1-2): 33-40.

### Example 37

### Preparation of Release Buffer

The release buffer is prepared by adding 8.22 g sodium chloride, 0.32 g sodium phosphate monobasic (monohydrate) and 2.60 g sodium phosphate dibasic (anhydrous) to a beaker. 1 L HPLC grade water is added and the solution is stirred until all the salts are dissolved. If required, the pH of the solution is adjusted to pH 7.4±0.2 using either 0.1N NaOH or 0.1N phosphoric acid.

### Example 38

### Release Study to Determine Release Profile of the Therapeutic Agent from a Coated Device

A sample of the therapeutic agent-loaded catheter is placed in a 15 ml culture tube. 15 ml release buffer (Example 38) is added to the culture tube. The tube is sealed with a TEFLON lined screw cap and is placed on a rotating wheel in a 37° C. oven. At various time points, the buffer is withdrawn from the culture tube and is replaced with fresh buffer. The withdrawn buffer is then analyzed for the amount of therapeutic agent contained in this buffer solution using HPLC.

### Example 39

### Screening Assay for Assessing the Effect of Paclitaxel on Cell Proliferation

Smooth muscle cells at 70-90% confluency were trypsinized, replated at 600 cells/well in media in 96-well plates and allowed to attachment overnight. Paclitaxel was prepared in DMSO at a concentration of 10⁻² M and diluted 10-fold to give a range of stock concentrations (10⁻⁸ M to 10⁻² M). Drug dilutions were diluted 1/1000 in media and added to cells to give a total volume of 200 μL/well. Each drug concentration was tested in triplicate wells. Plates containing cells and paclitaxel were incubated at 37° C. for 72 hours.

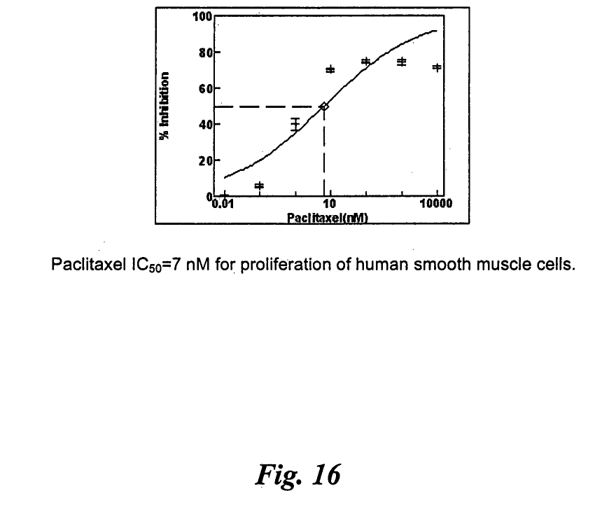

To terminate the assay, the media was removed by gentle aspiration. A 1/400 dilution of CYQUANT 400×GR dye indicator (Molecular Probes; Eugene, Oreg.) was added to 1× Cell Lysis buffer, and 200 μL of the mixture was added to the wells of the plate. Plates were incubated at room temperature, protected from light for 3-5 minutes. Fluorescence was read in a fluorescence microplate reader at ˜480 nm excitation wavelength and ˜520 nm emission maxima. Inhibitory concentration of 50% (IC₅₀) was determined by taking the average of triplicate wells and comparing average relative fluorescence units to the DMSO control. An average of n=3 replicate experiments was used to determine IC₅₀ values. See FIG. 16 (IC₅₀=7 nM). The IC₅₀ values for the following additional compounds were determined using this assay: IC₅₀ (nM): mycophenolic acid, 579; mofetil, 463; doxorubicin, 64; mitoxantrone, 1; geldanamycin, 5; anisomycin, 276; 17-AAG, 47; cytarabine, 85; halofuginone, 81; mitomycin C, 53; etoposide, 320; cladribine, 137; lovastatin, 978; epirubicin hydrochloride, 19; topotemay, 51; fascaplysin, 510; podophyllotoxin, 21; cytochalasin A, 221; gemcitabine, 9; puromycin, 384; mithramycin, 19; daunorubicin, 50; celastrol, 493; chromomycin A3, 12; vinorelbine, 15; idarubicin, 38; nogalamycin, 49; itraconazole, 795; 17-DMAG, 17; epothilone D, 5; tubercidin, 30; vinblastine, 3; vincristine, 9.

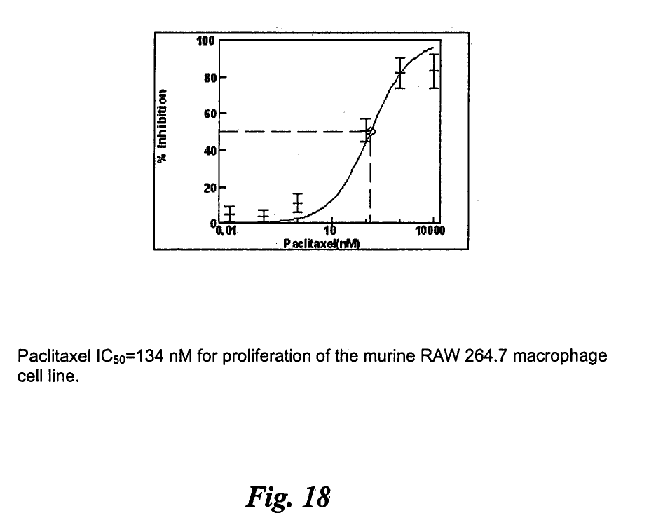

This assay also may be used assess the effect of compounds on proliferation of fibroblasts and murine macrophage cell line RAW 264.7. The results of the assay for assessing the effect of paclitaxel on proliferation of murine RAW 264.7 macrophage cell line were shown in FIG. 18 (IC₅₀=134 nM).

Reference: In vitro toxicol. (1990) 3: 219; Biotech. Histochem. (1993) 68: 29; Anal. Biochem. (1993) 213: 426.

### Example 40

### Perivascular Administration of Paclitaxel

WISTAR rats weighing 250-300 g are anesthetized by the intramuscular injection of Innovar (0.33 ml/kg). Once sedated, they are then placed under Halothane anesthesia. After general anesthesia is established, fur over the neck region is shaved, the skin clamped and swabbed with betadine. A vertical incision is made over the left carotid artery and the external carotid artery exposed. Two ligatures are placed around the external carotid artery and a transverse arteriotomy is made. A number 2 FRENCH FOGART balloon catheter is then introduced into the carotid artery and passed into the left common carotid artery and the balloon is inflated with saline. The catheter is passed up and down the carotid artery three times. The catheter is then removed and the ligature is tied off on the left external carotid artery.

Paclitaxel (33%) in ethelyne vinyl acetate (EVA) is then injected in a circumferential fashion around the common carotid artery in ten rats. EVA alone is injected around the common carotid artery in ten additional rats. (The paclitaxel may also be coated onto an EVA film which is then placed in a circumferential fashion around the common carotid artery.) Five rats from each group are sacrificed at 14 days and the final five at 28 days. The rats are observed for weight loss or other signs of systemic illness. After 14 or 28 days the animals are anesthetized and the left carotid artery is exposed in the manner of the initial experiment. The carotid artery is isolated, fixed at 10% buffered formaldehyde and examined for histology.

A statistically signifimayt reduction in the degree of initimal hyperplasia, as measured by standard morphometric analysis, indicates a drug induced reduction in fibrotic response.

### Example 41

### Complete Coating—Dip Coating a Vena Cava Filter

Poly(ethylene-co-vinyl acetate) {28% vinyl acetate} [p(EVA)] is dissolved in 10 ml THF to produce a solution that has a polymer concentration of approximately 40 mg/mL. Paclitaxel is added to the pEVA solution to produce a final paclitaxel concentration of 3 mg/mL. A vena cava filter is cleaned by immersing the filter into isopropanol for 30 minutes and then rinsing 3 times with isopropanol. The filter is air dried. The filter is dip coated by completely immersing the cleaned filter into the pEVA—paclitaxel solution. The filter is the removed from the solution and is air dried. This process may be repeated until the desired paclitaxel dose is achieved. The filter is then dried under vacuum. Other fibrosis-inhbiting agents that may be coated onto a vena cava filter device using this procedure include halofuginone, rapamycin, everolimus, and pimecerolimus.

### Example 42

### Partial Coating—Dip Coating a Vena Cava Filter

Polyurethane (CHRONOFLEX AL 85A) is dissolved in 10 ml THF to produce a solution that has a polymer concentration of approximately 400 mg/mL. Everolimus is added to the polyurethane solution to produce a final everolimus concentration of 3 mg/mL. A vena cava filter is cleaned by immersing the filter into isopropanol for 30 minutes and then rinsing 3 times with isopropanol. The filter is air dried. The filter is dip coated by immersing only the portions of the cleaned filter that will come into contact with the body tissue into the polyurethane—everolimus solution. The filter is the removed from the solution and is air dried. This process may be repeated until the desired everolimus dose is achieved. The filter is then dried under vacuum. Other fibrosis-inhbiting agents that may be coated onto a vena cava filter device using this procedure include halofuginone, rapamycin, paclitaxel, and pimecerolimus.

### Example 43

### Complete Coating—Spray Coating

A 2% solution poly(styrene-co-isobutylene-styrene) (SIBS) is prepared using THF as the solvent. Paclitaxel is added to the SIBS solution to produce a final paclitaxel concentration of 3 mg/mL. The SIBS—paclitaxel solution is then transferred to the reservoir of an artist's air brush tool. A vena cava filter is cleaned by immersing the filter into isopropanol for 30 minutes and then rinsing 3 times with isopropanol. The filter is air dried. Using a crocodile clip, the filter is suspended in the air and is spray coated from several angles to ensure complete coating of the filter. Once the coating is dry to the touch, the filter is removed from the clip and the uncoated portion is spray coated. The filter is then air dried and/or vacuum dried to remove the solvent. This process may be repeated until the desired paclitaxel dose is achieved. The filter is then dried under vacuum. Other fibrosis-inhbiting agents that may be coated onto a vena cava filter device using this procedure include halofuginone, rapamycin, everolimus, and pimecerolimus.

### Example 44

### Partial Coating—Spray Coating a Vena Cava Filter

A 2% solution poly(styrene-co-isobutylene-styrene) (SIBS) is prepared using THF as the solvent. Halofuginone is added to the SIBS solution to produce a final concentration of 3 mg/mL. The SIBS—halofuginone solution is then transferred to the reservoir of an artist's air brush tool. A vena cava filter is cleaned by immersing the filter into isopropanol for 30 minutes and then rinsing 3 times with isopropanol. The filter is air dried. Using a crocodile clip that is attached to a portion of the filter that is not to be coated, the filter is suspended in the air and is spray coated through a mask to ensure that only the desired portions of the filter are coated. The filter is then air dried and/or vacuum dried to remove the solvent. This process may be repeated until the desired halofuginone dose is achieved. The filter is then dried under vacuum. Other fibrosis-inhbiting agents that may be coated onto a vena cava filter device using this procedure include, paclitaxel, rapamycin, everolimus, and pimecerolimus.

### Example 45

### Application of a Second Coating to a Vena Cava Filter

Poly(ethylene-co-vinyl acetate) {28% vinyl acetate) [p(EVA)] is dissolved in 10 ml THF to produce a solution that has a polymer concentration of approximately 40 mg/mL. Halofuginone is added to the pEVA solution to produce a final halofuginone concentration of 3 mg/mL. A vena cava filter is cleaned by immersing the filter into isopropanol for 30 minutes and then rinsing 3 times with isopropanol. The filter is air dried. The filter is dip coated by completely immersing the cleaned filter into the pEVA—halofuginone solution. The filter is the removed from the solution and is air dried. This process may be repeated until the desired halofuginone dose is achieved. The filter is then dried under vacuum to remove the residual solvent. The filter is then dipped into an aqueous solution of sodium hyaluronate [HA] (mw approximately 1-1.5×10⁶ kDa, 10 mg/mL). The water is removed by air drying at 37° C. The process is repeated until the desired amount of HA is coated onto the filter. The filter is then dried under vacuum. Other fibrosis-inhbiting agents that may be coated onto a vena cava filter device using this procedure include, paclitaxel, rapamycin, everolimus, and pimecerolimus.

### Example 46

### Coating Containing Two Bioactive Agents for a Vena Cava Filter

Poly(ethylene-co-vinyl acetate) {28% vinyl acetate) [p(EVA)] is dissolved in 10 ml THF to produce a solution that has a polymer concentration of approximately 40 mg/mL. Paclitaxel is added to the pEVA solution to produce a final paclitaxel concentration of 3 mg/mL. Heparin-benzalkonium chloride is then added to the pEVA solution to achieve a final concentration of 1 mg/ml. A vena cava filter is cleaned by immersing the filter into isopropanol for 30 minutes and then rinsing 3 times with isopropanol. The filter is air dried. The filter is dip coated by completely immersing the cleaned filter into the pEVA—paclitaxel solution. The filter is the removed from the solution and is air dried. This process may be repeated until the desired paclitaxel dose is achieved. The filter is then dried under vacuum. Other fibrosis-inhbiting agents that may be coated onto a vena cava filter device using this procedure include, halofuginone, rapamycin, everolimus, and pimecerolimus.

### Example 47

### Two Coating Layers Containing Two Different Bioactive Agents for a Vena Cava Filter

Poly(ethylene-co-vinyl acetate) {28% vinyl acetate) [p(EVA)] is dissolved in 10 ml THF to produce a solution that has a polymer concentration of approximately 40 mg/mL. Rapamycin is added to the pEVA solution to produce a final Rapamycin concentration of 3 mg/mL. A vena cava filter is cleaned by immersing the filter into isopropanol for 30 minutes and then rinsing 3 times with isopropanol. The filter is air dried. The filter is dip coated by completely immersing the cleaned filter into the pEVA—rapamycin solution. The filter is the removed from the solution and is air dried. This process may be repeated until the desired rapamycin dose is achieved. The filter is then dried under vacuum to remove the residual solvent. The filter is then dipped into an aqueous solution of sodium hyaluronate [HA] (mw approximately 1-1.5×10⁶ kDa, 10 mg/mL) that contains 1 mg/ml heparin. The water is removed by air drying at 37° C. The process is repeated until the desired amount of HA is coated onto the filter. The filter is then dried under vacuum. Other fibrosis-inhbiting agents that may be coated onto a vena cava filter device using this procedure include, halofuginone, paclitaxel, everolimus, and pimecerolimus.

### Example 48

### Drug Incorporation into a Vascular Graft

A solution of halofuginone is prepared by dissolving 70 mg halofuginone in 10 mL water/ethanol (1:1) in a 20 mL glass scintillation vial. A 5 cm piece of a ePTFE vascular graft (IMPRA, 6 mm) is immersed in the solution. The solution is placed in an ultrasonic bath (Fisher) for 1 min. The graft is removed using a pair of tweezers. The graft is air dried for 3 hours after which it is dried under vacuum for 24 hours. Other fibrosis-inhbiting agents that may be coated onto a vascular graft device using this procedure include, rapamycin, paclitaxel, everolimus, and pimecerolimus.

### Example 49

### Drug Incorporation into a Tympanostomy Tube

Five 15 mL solutions of paclitaxel at 5 mg/ml are prepared in methanol in a 20 mL scintillation vial. A soft silicone T-tube ((Medco Catalogue Number T5030) is then immersed in each of the paclitaxel solutions. The tubes are removed from the paclitaxel solutions at 30 min, 1 hour, 2 hours, 6 hours and 24 hours. The tubes are air dried and then dried under vacuum for 24 hours. Other fibrosis-inhbiting agents that may be coated onto a tympanostomy tube device using this procedure include, rapamycin, halofuginone, everolimus, and pimecerolimus.

### Example 50

### Drug Incorporation into a Tympanostomy Tube

Five 15 mL solution of paclitaxel (5 mg/mL) and 5-fluorouracil (4 mg/mL) are prepared in methanol in a 20 mL scintillation vial. A soft silicone T-tube ((Medco Catalogue Number T5030) is then immersed in each of the paclitaxel solutions. The tubes are removed from the paclitaxel solutions at 30 min, 1 hour, 2 hours, 6 hours and 24 hours. The tubes are air dried and then dried under vacuum for 24 hours. Other fibrosis-inhbiting agents that may be coated onto a tympanostomy tube device using this procedure include, halofuginone, rapamycin, everolimus, and pimecerolimus.

### Example 51

### Drug Incorporation into an Intraocular Lens

Five 15 mL solution of paclitaxel (1 mg/mL) are prepared in methanol in a 20 mL scintillation vial. An intra-ocular lens (STAAR) then immersed in each of the paclitaxel solutions. The lenses are removed from the paclitaxel solutions at 30 min, 1 hour, 2 hours, 6 hours and 24 hours. The lenses are air dried and then dried under vacuum for 24 hours. Other fibrosis-inhbiting agents that may be coated onto an intraocular lens device using this procedure include, rapamycin, halofuginone, everolimus, and pimecerolimus.

The present invention also provides the following itemized embodiments:

1. A device, comprising an intravascular implant and an anti-scarring agent or a composition comprising an anti-scarring agent, wherein the agent inhibits scarring between the device and a host into which the device is implanted.

2. The device of item 1 wherein the agent inhibits cell regeneration.

3. The device of item 1 wherein the agent inhibits angiogenesis.

4. The device of item 1 wherein the agent inhibits fibroblast migration.

5. The device of item 1 wherein the agent inhibits fibroblast proliferation.

6. The device of item 1 wherein the agent inhibits deposition of extracellular matrix.

7. The device of item 1 wherein the agent inhibits tissue remodeling.

8. The device of item 1 wherein the agent is an angiogenesis inhibitor.

9. The device of item 1 wherein the agent is a 5-lipoxygenase inhibitor or antagonist.

10. The device of item 1 wherein the agent is a chemokine receptor antagonist.

11. The device of item 1 wherein the agent is a cell cycle inhibitor.

12. The device of item 1 wherein the agent is a taxane.

13. The device of item 1 wherein the agent is an anti-microtubule agent.

14. The device of item 1 wherein the agent is paclitaxel.

15. The device of item 1 wherein the agent is not paclitaxel.

16. The device of item 1 wherein the agent is an analogue or derivative of paclitaxel.

17. The device of item 1 wherein the agent is a vinca alkaloid.

18. The device of item 1 wherein the agent is camptothecin or an analogue or derivative thereof.

19. The device of item 1 wherein the agent is a podophyllotoxin.

20. The device of item 1 wherein the agent is a podophyllotoxin, wherein the podophyllotoxin is etoposide or an analogue or derivative thereof.

21. The device of item 1 wherein the agent is an anthracycline.

22. The device of item 1 wherein the agent is an anthracycline, wherein the anthracycline is doxorubicin or an analogue or derivative thereof.

23. The device of item 1 wherein the agent is an anthracycline, wherein the anthracycline is mitoxantrone or an analogue or derivative thereof.

24. The device of item 1 wherein the agent is a platinum compound.

25. The device of item 1 wherein the agent is a nitrosourea.

26. The device of item 1 wherein the agent is a nitroimidazole.

27. The device of item 1 wherein the agent is a folic acid antagonist.

28. The device of item 1 wherein the agent is a cytidine analogue.

29. The device of item 1 wherein the agent is a pyrimidine analogue.

30. The device of item 1 wherein the agent is a fluoropyrimidine analogue.

31. The device of item 1 wherein the agent is a purine analogue.

32. The device of item 1 wherein the agent is a nitrogen mustard or an analogue or derivative thereof.

33. The device of item 1 wherein the agent is a hydroxyurea.

34. The device of item 1 wherein the agent is a mytomicin or an analogue or derivative thereof.

35. The device of item 1 wherein the agent is an alkyl sulfonate.

36. The device of item 1 wherein the agent is a benzamide or an analogue or derivative thereof.

37. The device of item 1 wherein the agent is a nicotinamide or an analogue or derivative thereof.

38. The device of item 1 wherein the agent is a halogenated sugar or an analogue or derivative thereof.

39. The device of item 1 wherein the agent is a DNA alkylating agent.

40. The device of item 1 wherein the agent is an anti-microtubule agent.

41. The device of item 1 wherein the agent is a topoisomerase inhibitor.

42. The device of item 1 wherein the agent is a DNA cleaving agent.

43. The device of item 1 wherein the agent is an antimetabolite.

44. The device of item 1 wherein the agent inhibits adenosine deaminase.

45. The device of item 1 wherein the agent inhibits purine ring synthesis.

46. The device of item 1 wherein the agent is a nucleotide interconversion inhibitor.

47. The device of item 1 wherein the agent inhibits dihydrofolate reduction.

48. The device of item 1 wherein the agent blocks thymidine monophosphate.

49. The device of item 1 wherein the agent causes DNA damage.

50. The device of item 1 wherein the agent is a DNA intercalation agent.

51. The device of item 1 wherein the agent is a RNA synthesis inhibitor.

52. The device of item 1 wherein the agent is a pyrimidine synthesis inhibitor.

53. The device of item 1 wherein the agent inhibits ribonucleotide synthesis or function.

54. The device of item 1 wherein the agent inhibits thymidine monophosphate synthesis or function.

55. The device of item 1 wherein the agent inhibits DNA synthesis.

56. The device of item 1 wherein the agent causes DNA adduct formation.

57. The device of item 1 wherein the agent inhibits protein synthesis.

58. The device of item 1 wherein the agent inhibits microtubule function.

59. The device of item 1 wherein the agent is a cyclin dependent protein kinase inhibitor.

60. The device of item 1 wherein the agent is an epidermal growth factor kinase inhibitor.

61. The device of item 1 wherein the agent is an elastase inhibitor.

62. The device of item 1 wherein the agent is a factor Xa inhibitor.

63. The device of item 1 wherein the agent is a farnesyltransferase inhibitor.

64. The device of item 1 wherein the agent is a fibrinogen antagonist.

65. The device of item 1 wherein the agent is a guanylate cyclase stimulant.

66. The device of item 1 wherein the agent is a heat shock protein 90 antagonist.

67. The device of item 1 wherein the agent is a heat shock protein 90 antagonist, wherein the heat shock protein 90 antagonist is geldanamycin or an analogue or derivative thereof.

68. The device of item 1 wherein the agent is a guanylate cyclase stimulant.

69. The device of item 1 wherein the agent is a HMGCoA reductase inhibitor.

70. The device of item 1 wherein the agent is a HMGCoA reductase inhibitor, wherein the HMGCoA reductase inhibitor is simvastatin or an analogue or derivative thereof.

71. The device of item 1 wherein the agent is a hydroorotate dehydrogenase inhibitor.

72. The device of item 1 wherein the agent is an IKK2 inhibitor.

73. The device of item 1 wherein the agent is an IL-1 antagonist.

74. The device of item 1 wherein the agent is an ICE antagonist.

75. The device of item 1 wherein the agent is an IRAK antagonist.

76. The device of item 1 wherein the agent is an IL-4 agonist.

77. The device of item 1 wherein the agent is an immunomodulatory agent.

78. The device of item 1 wherein the agent is sirolimus or an analogue or derivative thereof.

79. The device of item 1 wherein the agent is not sirolimus.

80. The device of item 1 wherein the agent is everolimus or an analogue or derivative thereof.

81. The device of item 1 wherein the agent is tacrolimus or an analogue or derivative thereof.

82. The device of item 1 wherein the agent is not tacrolimus.

83. The device of item 1 wherein the agent is biolmus or an analogue or derivative thereof.

84. The device of item 1 wherein the agent is tresperimus or an analogue or derivative thereof.

85. The device of item 1 wherein the agent is auranofin or an analogue or derivative thereof.

86. The device of item 1 wherein the agent is 27-0-demethylrapamycin or an analogue or derivative thereof.

87. The device of item 1 wherein the agent is gusperimus or an analogue or derivative thereof.

88. The device of item 1 wherein the agent is pimecrolimus or an analogue or derivative thereof.

89. The device of item 1 wherein the agent is ABT-578 or an analogue or derivative thereof.

90. The device of item 1 wherein the agent is an inosine monophosphate dehydrogenase (IMPDH) inhibitor.

91. The device of item 1 wherein the agent is an IMPDH inhibitor, wherein the IMPDH inhibitor is mycophenolic acid or an analogue or derivative thereof.

92. The device of item 1 wherein the agent is an IMPDH inhibitor, wherein the IMPDH inhibitor is 1-alpha-25 dihydroxy vitamin D3 or an analogue or derivative thereof.

93. The device of item 1 wherein the agent is a leukotriene inhibitor.

94. The device of item 1 wherein the agent is a MCP-1 antagonist.

95. The device of item 1 wherein the agent is a MMP inhibitor.

96. The device of item 1 wherein the agent is an NF kappa B inhibitor.

97. The device of item 1 wherein the agent is an NF kappa B inhibitor, wherein the NF kappa B inhibitor is Bay 11-7082.

98. The device of item 1 wherein the agent is an NO agonist.

99. The device of item 1 wherein the agent is a p38 MAP kinase inhibitor.

100. The device of item 1 wherein the agent is a p38 MAP kinase inhibitor, wherein the p38 MAP kinase inhibitor is SB 202190.

101. The device of item 1 wherein the agent is a phosphodiesterase inhibitor.

102. The device of item 1 wherein the agent is a TGF beta inhibitor.

103. The device of item 1 wherein the agent is a thromboxane A2 antagonist.

104. The device of item 1 wherein the agent is a TNFa antagonist.

105. The device of item 1 wherein the agent is a TACE inhibitor.

106. The device of item 1 wherein the agent is a tyrosine kinase inhibitor.

107. The device of item 1 wherein the agent is a vitronectin inhibitor.

108. The device of item 1 wherein the agent is a fibroblast growth factor inhibitor.

109. The device of item 1 wherein the agent is a protein kinase inhibitor.

110. The device of item 1 wherein the agent is a PDGF receptor kinase inhibitor.

111. The device of item 1 wherein the agent is an endothelial growth factor receptor kinase inhibitor.

112. The device of item 1 wherein the agent is a retinoic acid receptor antagonist.

113. The device of item 1 wherein the agent is a platelet derived growth factor receptor kinase inhibitor.

114. The device of item 1 wherein the agent is a fibronogin antagonist.

115. The device of item 1 wherein the agent is an antimycotic agent.

116. The device of item 1 wherein the agent is an antimycotic agent, wherein the antimycotic agent is sulconizole.

117. The device of item 1 wherein the agent is a bisphosphonate.

118. The device of item 1 wherein the agent is a phospholipase A1 inhibitor.

119. The device of item 1 wherein the agent is a histamine H1/H2/H3 receptor antagonist.

120. The device of item 1 wherein the agent is a macrolide antibiotic.

121. The device of item 1 wherein the agent is a GPIIb/IIIa receptor antagonist.

122. The device of item 1 wherein the agent is an endothelin receptor antagonist.

123. The device of item 1 wherein the agent is a peroxisome proliferator-activated receptor agonist.

124. The device of item 1 wherein the agent is an estrogen receptor agent.

125. The device of item 1 wherein the agent is a somastostatin analogue.

126. The device of item 1 wherein the agent is a neurokinin 1 antagonist.

127. The device of item 1 wherein the agent is a neurokinin 3 antagonist.

128. The device of item 1 wherein the agent is a VLA-4 antagonist.

129. The device of item 1 wherein the agent is an osteoclast inhibitor.

130. The device of item 1 wherein the agent is a DNA topoisomerase ATP hydrolyzing inhibitor.

131. The device of item 1 wherein the agent is an angiotensin I converting enzyme inhibitor.

132. The device of item 1 wherein the agent is an angiotensin II antagonist.

133. The device of item 1 wherein the agent is an enkephalinase inhibitor.

134. The device of item 1 wherein the agent is a peroxisome proliferator-activated receptor gamma agonist insulin sensitizer.

135. The device of item 1 wherein the agent is a protein kinase C inhibitor.

136. The device of item 1 wherein the agent is a ROCK (rho-associated kinase) inhibitor.

137. The device of item 1 wherein the agent is a CXCR3 inhibitor.

138. The device of item 1 wherein the agent is an Itk inhibitor.

139. The device of item 1 wherein the agent is a cytosolic phospholipase A2-alpha inhibitor.

140. The device of item 1 wherein the agent is a PPAR agonist.

141. The device of item 1 wherein the agent is an immunosuppressant.

142. The device of item 1 wherein the agent is an Erb inhibitor.

143. The device of item 1 wherein the agent is an apoptosis agonist.

144. The device of item 1 wherein the agent is a lipocortin agonist.

145. The device of item 1 wherein the agent is a VCAM-1 antagonist.

146. The device of item 1 wherein the agent is a collagen antagonist.

147. The device of item 1 wherein the agent is an alpha 2 integrin antagonist.

148. The device of item 1 wherein the agent is a TNF alpha inhibitor.

149. The device of item 1 wherein the agent is a nitric oxide inhibitor.

150. The device of item 1 wherein the agent is a cathepsin inhibitor.

151. The device of item 1 wherein the agent is not an anti-inflammatory agent.

152. The device of item 1 wherein the agent is not a steroid.

153. The device of item 1 wherein the agent is not a glucocorticosteroid.

154. The device of item 1 wherein the agent is not dexamethasone.

155. The device of item 1 wherein the agent is not an anti-infective agent.

156. The device of item 1 wherein the agent is not an antibiotic.

157. The device of item 1 wherein the agent is not an anti-fungal agent.

158. The device of item 1, further comprising a polymer.

159. The device of item 1, further comprising a polymeric carrier.

160. The device of item 1 wherein the anti-scarring agent inhibits adhesion between the device and a host into which the device is implanted.

161. The device of item 1 wherein the device delivers the anti-scarring agent locally to tissue proximate to the device.

162. The device of item 1, further comprising a coating, wherein the coating comprises the anti-scarring agent.

163. The device of item 1, further comprising a coating, wherein the coating is disposed on a surface of the device.

164. The device of item 1, further comprising a coating, wherein the coating directly contacts the device.

165. The device of item 1, further comprising a coating, wherein the coating indirectly contacts the device.

166. The device of item 1, further comprising a coating, wherein the coating partially covers the device.

167. The device of item 1, further comprising a coating, wherein the coating completely covers the device.

168. The device of item 1, further comprising a coating, wherein the coating is a uniform coating.

169. The device of item 1, further comprising a coating, wherein the coating is a non-uniform coating.

170. The device of item 1, further comprising a coating, wherein the coating is a discontinuous coating.

171. The device of item 1, further comprising a coating, wherein the coating is a patterned coating.

172. The device of item 1, further comprising a coating, wherein the coating has a thickness of 100 μm or less.

173. The device of item 1, further comprising a coating, wherein the coating has a thickness of 10 μm or less.

174. The device of item 1, further comprising a coating, wherein the coating adheres to the surface of the device upon deployment of the device.

175. The device of item 1, further comprising a coating, wherein the coating is stable at room temperature for a period of 1 year.

176. The device of item 1, further comprising a coating, wherein the anti-scarring agent is present in the coating in an amount ranging between about 0.0001% to about 1% by weight.

177. The device of item 1, further comprising a coating, wherein the anti-scarring agent is present in the coating in an amount ranging between about 1% to about 10% by weight.

178. The device of item 1, further comprising a coating, wherein the anti-scarring agent is present in the coating in an amount ranging between about 10% to about 25% by weight.

179. The device of item 1, further comprising a coating, wherein the anti-scarring agent is present in the coating in an amount ranging between about 25% to about 70% by weight.

180. The device of item 1, further comprising a coating, wherein the coating further comprises a polymer.

181. The device of item 1, further comprising a first coating having a first composition and the second coating having a second composition.

182. The device of item 1, further comprising a first coating having a first composition and the second coating having a second composition, wherein the first composition and the second composition are different.

183. The device of item 1, further comprising a polymer.

184. The device of item 1, further comprising a polymeric carrier.

185. The device of item 1, further comprising a polymeric carrier, wherein the polymeric carrier comprises a copolymer.

186. The device of item 1, further comprising a polymeric carrier, wherein the polymeric carrier comprises a block copolymer.

187. The device of item 1, further comprising a polymeric carrier, wherein the polymeric carrier comprises a random copolymer.

188. The device of item 1, further comprising a polymeric carrier, wherein the polymeric carrier comprises a biodegradable polymer.

189. The device of item 1, further comprising a polymeric carrier, wherein the polymeric carrier comprises a non-biodegradable polymer.

190. The device of item 1, further comprising a polymeric carrier, wherein the polymeric carrier comprises a hydrophilic polymer.

191. The device of item 1, further comprising a polymeric carrier, wherein the polymeric carrier comprises a hydrophobic polymer.

192. The device of item 1, further comprising a polymeric carrier, wherein the polymeric carrier comprises a polymer having hydrophilic domains.

193. The device of item 1, further comprising a polymeric carrier, wherein the polymeric carrier comprises a polymer having hydrophobic domains.

194. The device of item 1, further comprising a polymeric carrier, wherein the polymeric carrier comprises a non-conductive polymer.

195. The device of item 1, further comprising a polymeric carrier, wherein the polymeric carrier comprises an elastomer.

196. The device of item 1, further comprising a polymeric carrier, wherein the polymeric carrier comprises a hydrogel.

197. The device of item 1, further comprising a polymeric carrier, wherein the polymeric carrier comprises a silicone polymer.

198. The device of item 1, further comprising a polymeric carrier, wherein the polymeric carrier comprises a hydrocarbon polymer.

199. The device of item 1, further comprising a polymeric carrier, wherein the polymeric carrier comprises a styrene-derived polymer.

200. The device of item 1, further comprising a polymeric carrier, wherein the polymeric carrier comprises a butadiene polymer.

201. The device of item 1, further comprising a polymeric carrier, wherein the polymeric carrier comprises a macromer.

202. The device of item 1, further comprising a polymeric carrier, wherein the polymeric carrier comprises a poly(ethylene glycol)polymer.

203. The device of item 1, further comprising a polymeric carrier, wherein the polymeric carrier comprises an amorphous polymer.

204. The device of item 1, further comprising a lubricious coating.

205. The device of item 1 wherein the anti-scarring agent is located within pores or holes of the device.

206. The device of item 1 wherein the anti-scarring agent is located within a channel, lumen, or divet of the device.

207. The device of item 1, further comprising a second pharmaceutically active agent.

208. The device of item 1, further comprising an anti-inflammatory agent.

209. The device of item 1, further comprising an agent that inhibits infection.

210. The device of item 1, further comprising an agent that inhibits infection, wherein the agent is an anthracycline.

211. The device of item 1, further comprising an agent that inhibits infection, wherein the agent is doxorubicin.

212. The device of item 1, further comprising an agent that inhibits infection, wherein the agent is mitoxantrone.

213. The device of item 1, further comprising an agent that inhibits infection, wherein the agent is a fluoropyrimidine.

214. The device of item 1, further comprising an agent that inhibits infection, wherein the agent is 5-fluorouracil (5-FU).

215. The device of item 1, further comprising an agent that inhibits infection, wherein the agent is a folic acid antagonist.

216. The device of item 1, further comprising an agent that inhibits infection, wherein the agent is methotrexate.

217. The device of item 1, further comprising an agent that inhibits infection, wherein the agent is a podophylotoxin.

218. The device of item 1, further comprising an agent that inhibits infection, wherein the agent is etoposide.

219. The device of item 1, further comprising an agent that inhibits infection, wherein the agent is a camptothecin.

220. The device of item 1, further comprising an agent that inhibits infection, wherein the agent is a hydroxyurea.

221. The device of item 1, further comprising an agent that inhibits infection, wherein the agent is a platinum complex.

222. The device of item 1, further comprising an agent that inhibits infection, wherein the agent is cisplatin.

223. The device of item 1, further comprising an anti-thrombotic agent.

224. The device of item 1, further comprising a visualization agent.

225. The device of item 1, further comprising a visualization agent, wherein the visualization agent is a radiopaque material, wherein the radiopaque material comprises a metal, a halogenated compound, or a barium containing compound.

226. The device of item 1, further comprising a visualization agent, wherein the visualization agent is a radiopaque material, wherein the radiopaque material comprises barium, tantalum, or technetium.

227. The device of item 1, further comprising a visualization agent, wherein the visualization agent is a MRI responsive material.

228. The device of item 1, further comprising a visualization agent, wherein the visualization agent comprises a gadolinium chelate.

229. The device of item 1, further comprising a visualization agent, wherein the visualization agent comprises iron, magnesium, manganese, copper, or chromium.

230. The device of item 1, further comprising a visualization agent, wherein the visualization agent comprises an iron oxide compound.

231. The device of item 1, further comprising a visualization agent, wherein the visualization agent comprises a dye, pigment, or colorant.

232. The device of item 1, further comprising an echogenic material.

233. The device of item 1, further comprising an echogenic material, wherein the echogenic material is in the form of a coating.

234. The device of item 1 wherein the device is sterile.

235. The device of item 1 wherein the anti-scarring agent is released into tissue in the vicinity of the device after deployment of the device.

236. The device of item 1 wherein the anti-scarring agent is released into tissue in the vicinity of the device after deployment of the device, wherein the tissue is connective tissue.

237. The device of item 1 wherein the anti-scarring agent is released into tissue in the vicinity of the device after deployment of the device, wherein the tissue is muscle tissue.

238. The device of item 1 wherein the anti-scarring agent is released into tissue in the vicinity of the device after deployment of the device, wherein the tissue is nerve tissue.

239. The device of item 1 wherein the anti-scarring agent is released into tissue in the vicinity of the device after deployment of the device, wherein the tissue is epithelium tissue.

240. The device of item 1 wherein the anti-scarring agent is released in effective concentrations from the device over a period ranging from the time of deployment of the device to about 1 year.

241. The device of item 1 wherein the anti-scarring agent is released in effective concentrations from the device over a period ranging from about 1 month to 6 months.

242. The device of item 1 wherein the anti-scarring agent is released in effective concentrations from the device over a period ranging from about 1-90 days.

243. The device of item 1 wherein the anti-scarring agent is released in effective concentrations from the device at a constant rate.

244. The device of item 1 wherein the anti-scarring agent is released in effective concentrations from the device at an increasing rate.

245. The device of item 1 wherein the anti-scarring agent is released in effective concentrations from the device at a decreasing rate.

246. The device of item 1 wherein the anti-scarring agent is released in effective concentrations from the composition comprising the anti-scarring agent by diffusion over a period ranging from the time of deployment of the device to about 90 days.

247. The device of item 1 wherein the anti-scarring agent is released in effective concentrations from the composition comprising the anti-scarring agent by erosion of the composition over a period ranging from the time of deployment of the device to about 90 days.

248. The device of item 1 wherein the device comprises about 0.01 μg to about 10 μg of the anti-scarring agent.

249. The device of item 1 wherein the device comprises about 10 μg to about 10 mg of the anti-scarring agent.

250. The device of item 1 wherein the device comprises about 10 mg to about 250 mg of the anti-scarring agent.

251. The device of item 1 wherein the device comprises about 250 mg to about 1000 mg of the anti-scarring agent.

252. The device of item 1 wherein the device comprises about 1000 mg to about 2500 mg of the anti-scarring agent.

253. The device of item 1 wherein a surface of the device comprises less than 0.01 μg of the anti-scarring agent per mm2 of device surface to which the anti-scarring agent is applied.

254. The device of item I wherein a surface of the device comprises about 0.01 μg to about 1 μg of the anti-scarring agent per mm2 of device surface to which the anti-scarring agent is applied.

255. The device of item 1 wherein a surface of the device comprises about 1 μg to about 10 μg of the anti-scarring agent per mm2 of device surface to which the anti-scarring agent is applied.

256. The device of item 1 wherein a surface of the device comprises about 10 μg to about 250 μg of the anti-scarring agent per mm2 of device surface to which the anti-scarring agent is applied.

257. The device of item 1 wherein a surface of the device comprises about 250 μg to about 1000 μg of the anti-scarring agent of anti-scarring agent per mm2 of device surface to which the anti-scarring agent is applied.

258. The device of item 1 wherein a surface of the device comprises about 1000 μg to about 2500 μg of the anti-scarring agent per mm2 of device surface to which the anti-scarring agent is applied.

259. The device of any one of items 1-258 wherein the implant is a stent.

260. The device of any one of items 1-258 wherein the implant is a coronary stent.

261. The device of any one of items 1-258 wherein the implant is a peripheral stent.

262. The device of any one of items 1-258 wherein the implant is a covered stent.

263. The device of any one of items 1-258 wherein the implant is an intravascular catheter.

264. The device of any one of items 1-258 wherein the implant is a microinjector catheter.

265. The device of any one of items 1-258 wherein the implant is a drug delivery balloon.

266. The device of any one of items 1-258 wherein the implant is a sweaty balloon.

267. The device of any one of items 1-258 wherein the implant is a channel balloon.

268. The device of any one of items 1-258 wherein the implant is a microinjector balloon.

269. The device of any one of items 1-258 wherein the implant is a double balloon.

270. The device of any one of items 1-258 wherein the implant is a spiral balloon.

271. The device of any one of items 1-258 wherein the implant is a BHP balloon.

272. The device of any one of items 1-258 wherein the implant is a transurethral needle ablation (TUNA) balloon.

273. The device of any one of items 1-258 wherein the implant is a radio frequency needle ablation (RFNA) balloon.

274. The device of any one of items 1-258 wherein the implant is a coronary drug infuction guidewire.

275. A device, comprising a vascular graft or wrap implant and an anti-scarring agent or a composition comprising an anti-scarring agent, wherein the agent inhibits scarring between the device and a host into which the device is implanted.

276. The device of item 275 wherein the agent inhibits cell regeneration.

277. The device of item 275 wherein the agent inhibits angiogenesis.

278. The device of item 275 wherein the agent inhibits fibroblast migration.

279. The device of item 275 wherein the agent inhibits fibroblast proliferation.

280. The device of item 275 wherein the agent inhibits deposition of extracellular matrix.

281. The device of item 275 wherein the agent inhibits tissue remodeling.

282. The device of item 275 wherein the agent is an angiogenesis inhibitor.

283. The device of item 275 wherein the agent is a 5-lipoxygenase inhibitor or antagonist.

284. The device of item 275 wherein the agent is a chemokine receptor antagonist.

285. The device of item 275 wherein the agent is a cell cycle inhibitor.

286. The device of item 275 wherein the agent is a taxane.

287. The device of item 275 wherein the agent is an anti-microtubule agent.

288. The device of item 275 wherein the agent is paclitaxel.

289. The device of item 275 wherein the agent is not paclitaxel.

290. The device of item 275 wherein the agent is an analogue or derivative of paclitaxel.

291. The device of item 275 wherein the agent is a vinca alkaloid.

292. The device of item 275 wherein the agent is camptothecin or an analogue or derivative thereof.

293. The device of item 275 wherein the agent is a podophyllotoxin.

294. The device of item 275 wherein the agent is a podophyllotoxin, wherein the podophyllotoxin is etoposide or an analogue or derivative thereof.

295. The device of item 275 wherein the agent is an anthracycline.

296. The device of item 275 wherein the agent is an anthracycline, wherein the anthracycline is doxorubicin or an analogue or derivative thereof.

297. The device of item 275 wherein the agent is an anthracycline, wherein the anthracycline is mitoxantrone or an analogue or derivative thereof.

298. The device of item 275 wherein the agent is a platinum compound.

299. The device of item 275 wherein the agent is a nitrosourea.

300. The device of item 275 wherein the agent is a nitroimidazole.

301. The device of item 275 wherein the agent is a folic acid antagonist.

302. The device of item 275 wherein the agent is a cytidine analogue.

303. The device of item 275 wherein the agent is a pyrimidine analogue.

304. The device of item 275 wherein the agent is a fluoropyrimidine analogue.

305. The device of item 275 wherein the agent is a purine analogue.

306. The device of item 275 wherein the agent is a nitrogen mustard or an analogue or derivative thereof.

307. The device of item 275 wherein the agent is a hydroxyurea.

308. The device of item 275 wherein the agent is a mytomicin or an analogue or derivative thereof.

309. The device of item 275 wherein the agent is an alkyl sulfonate.

310. The device of item 275 wherein the agent is a benzamide or an analogue or derivative thereof.

311. The device of item 275 wherein the agent is a nicotinamide or an analogue or derivative thereof.

312. The device of item 275 wherein the agent is a halogenated sugar or an analogue or derivative thereof.

313. The device of item 275 wherein the agent is a DNA alkylating agent.

314. The device of item 275 wherein the agent is an anti-microtubule agent.

315. The device of item 275 wherein the agent is a topoisomerase inhibitor.

316. The device of item 275 wherein the agent is a DNA cleaving agent.

317. The device of item 275 wherein the agent is an antimetabolite.

318. The device of item 275 wherein the agent inhibits adenosine deaminase.

319. The device of item 275 wherein the agent inhibits purine ring synthesis.

320. The device of item 275 wherein the agent is a nucleotide interconversion inhibitor.

321. The device of item 275 wherein the agent inhibits dihydrofolate reduction.

322. The device of item 275 wherein the agent blocks thymidine monophosphate.

323. The device of item 275 wherein the agent causes DNA damage.

324. The device of item 275 wherein the agent is a DNA intercalation agent.

325. The device of item 275 wherein the agent is a RNA synthesis inhibitor.

326. The device of item 275 wherein the agent is a pyrimidine synthesis inhibitor.

327. The device of item 275 wherein the agent inhibits ribonucleotide synthesis or function.

328. The device of item 275 wherein the agent inhibits thymidine monophosphate synthesis or function.

329. The device of item 275 wherein the agent inhibits DNA synthesis.

330. The device of item 275 wherein the agent causes DNA adduct formation.

331. The device of item 275 wherein the agent inhibits protein synthesis.

332. The device of item 275 wherein the agent inhibits microtubule function.

333. The device of item 275 wherein the agent is a cyclin dependent protein kinase inhibitor.

334. The device of item 275 wherein the agent is an epidermal growth factor kinase inhibitor.

335. The device of item 275 wherein the agent is an elastase inhibitor.

336. The device of item 275 wherein the agent is a factor Xa inhibitor.

337. The device of item 275 wherein the agent is a farnesyltransferase inhibitor.

338. The device of item 275 wherein the agent is a fibrinogen antagonist.

339. The device of item 275 wherein the agent is a guanylate cyclase stimulant.

340. The device of item 275 wherein the agent is a heat shock protein 90 antagonist.

341. The device of item 275 wherein the agent is a heat shock protein 90 antagonist, wherein the heat shock protein 90 antagonist is geldanamycin or an analogue or derivative thereof.

342. The device of item 275 wherein the agent is a guanylate cyclase stimulant.

343. The device of item 275 wherein the agent is a HMGCoA reductase inhibitor.

344. The device of item 275 wherein the agent is a HMGCoA reductase inhibitor, wherein the HMGCoA reductase inhibitor is simvastatin or an analogue or derivative thereof.

345. The device of item 275 wherein the agent is a hydroorotate dehydrogenase inhibitor.

346. The device of item 275 wherein the agent is an IKK2 inhibitor.

347. The device of item 275 wherein the agent is an IL-1 antagonist.

348. The device of item 275 wherein the agent is an ICE antagonist.

349. The device of item 275 wherein the agent is an IRAK antagonist.

350. The device of item 275 wherein the agent is an IL-4 agonist.

351. The device of item 275 wherein the agent is an immunomodulatory agent.

352. The device of item 275 wherein the agent is sirolimus or an analogue or derivative thereof.

353. The device of item 275 wherein the agent is not sirolimus.

354. The device of item 275 wherein the agent is everolimus or an analogue or derivative thereof.

355. The device of item 275 wherein the agent is tacrolimus or an analogue or derivative thereof.

356. The device of item 275 wherein the agent is not tacrolimus.

357. The device of item 275 wherein the agent is biolmus or an analogue or derivative thereof.

358. The device of item 275 wherein the agent is tresperimus or an analogue or derivative thereof.

359. The device of item 275 wherein the agent is auranofin or an analogue or derivative thereof.

360. The device of item 275 wherein the agent is 27-0-demethylrapamycin or an analogue or derivative thereof.

361. The device of item 275 wherein the agent is gusperimus or an analogue or derivative thereof.

362. The device of item 275 wherein the agent is pimecrolimus or an analogue or derivative thereof.

363. The device of item 275 wherein the agent is ABT-578 or an analogue or derivative thereof.

364. The device of item 275 wherein the agent is an inosine monophosphate dehydrogenase (IMPDH) inhibitor.

365. The device of item 275 wherein the agent is an IMPDH inhibitor, wherein the IMPDH inhibitor is mycophenolic acid or an analogue or derivative thereof.

366. The device of item 275 wherein the agent is an IMPDH inhibitor, wherein the IMPDH inhibitor is 1-alpha-25 dihydroxy vitamin D3 or an analogue or derivative thereof.

367. The device of item 275 wherein the agent is a leukotriene inhibitor.

368. The device of item 275 wherein the agent is a MCP-1 antagonist.

369. The device of item 275 wherein the agent is a MMP inhibitor.

370. The device of item 275 wherein the agent is an NF kappa B inhibitor.

371. The device of item 275 wherein the agent is an NF kappa B inhibitor, wherein the NF kappa B inhibitor is Bay 11-7082.

372. The device of item 275 wherein the agent is an NO agonist.

373. The device of item 275 wherein the agent is a p38 MAP kinase inhibitor.

374. The device of item 275 wherein the agent is a p38 MAP kinase inhibitor, wherein the p38 MAP kinase inhibitor is SB 202190.

375. The device of item 275 wherein the agent is a phosphodiesterase inhibitor.

376. The device of item 275 wherein the agent is a TGF beta inhibitor.

377. The device of item 275 wherein the agent is a thromboxane A2 antagonist.

378. The device of item 275 wherein the agent is a TNFa antagonist.

379. The device of item 275 wherein the agent is a TACE inhibitor.

380. The device of item 275 wherein the agent is a tyrosine kinase inhibitor.

381. The device of item 275 wherein the agent is a vitronectin inhibitor.

382. The device of item 275 wherein the agent is a fibroblast growth factor inhibitor.

383. The device of item 275 wherein the agent is a protein kinase inhibitor.

384. The device of item 275 wherein the agent is a PDGF receptor kinase inhibitor.

385. The device of item 275 wherein the agent is an endothelial growth factor receptor kinase inhibitor.

386. The device of item 275 wherein the agent is a retinoic acid receptor antagonist.

387. The device of item 275 wherein the agent is a platelet derived growth factor receptor kinase inhibitor.

388. The device of item 275 wherein the agent is a fibronogin antagonist.

389. The device of item 275 wherein the agent is an antimycotic agent.

390. The device of item 275 wherein the agent is an antimycotic agent, wherein the antimycotic agent is sulconizole.

391. The device of item 275 wherein the agent is a bisphosphonate.

392. The device of item 275 wherein the agent is a phospholipase A1 inhibitor.

393. The device of item 275 wherein the agent is a histamine H1/H2/H3 receptor antagonist.

394. The device of item 275 wherein the agent is a macrolide antibiotic.

395. The device of item 275 wherein the agent is a GPIIb/IIIa receptor antagonist.

396. The device of item 275 wherein the agent is an endothelin receptor antagonist.

397. The device of item 275 wherein the agent is a peroxisome proliferator-activated receptor agonist.

398. The device of item 275 wherein the agent is an estrogen receptor agent.

399. The device of item 275 wherein the agent is a somastostatin analogue.

400. The device of item 275 wherein the agent is a neurokinin 1 antagonist.

401. The device of item 275 wherein the agent is a neurokinin 3 antagonist.

402. The device of item 275 wherein the agent is a VLA-4 antagonist.

403. The device of item 275 wherein the agent is an osteoclast inhibitor.

404. The device of item 275 wherein the agent is a DNA topoisomerase ATP hydrolyzing inhibitor.

405. The device of item 275 wherein the agent is an angiotensin I converting enzyme inhibitor.

406. The device of item 275 wherein the agent is an angiotensin II antagonist.

407. The device of item 275 wherein the agent is an enkephalinase inhibitor.

408. The device of item 275 wherein the agent is a peroxisome proliferator-activated receptor gamma agonist insulin sensitizer.

409. The device of item 275 wherein the agent is a protein kinase C inhibitor.

410. The device of item 275 wherein the agent is a ROCK (rho-associated kinase) inhibitor.

411. The device of item 275 wherein the agent is a CXCR3 inhibitor.

412. The device of item 275 wherein the agent is an Itk inhibitor.

413. The device of item 275 wherein the agent is a cytosolic phospholipase A2-alpha inhibitor.

414. The device of item 275 wherein the agent is a PPAR agonist.

415. The device of item 275 wherein the agent is an immunosuppressant.

416. The device of item 275 wherein the agent is an Erb inhibitor.

417. The device of item 275 wherein the agent is an apoptosis agonist.

418. The device of item 275 wherein the agent is a lipocortin agonist.

419. The device of item 275 wherein the agent is a VCAM-1 antagonist.

420. The device of item 275 wherein the agent is a collagen antagonist.

421. The device of item 275 wherein the agent is an alpha 2 integrin antagonist.

422. The device of item 275 wherein the agent is a TNF alpha inhibitor.

423. The device of item 275 wherein the agent is a nitric oxide inhibitor 424. The device of item 275 wherein the agent is a cathepsin inhibitor.

425. The device of item 275 wherein the agent is not an anti-inflammatory agent.

426. The device of item 275 wherein the agent is not a steroid.

427. The device of item 275 wherein the agent is not a glucocorticosteroid.

428. The device of item 275 wherein the agent is not dexamethasone.

429. The device of item 275 wherein the agent is not an anti-infective agent.

430. The device of item 275 wherein the agent is not an antibiotic.

431. The device of item 275 wherein the agent is not an anti-fungal agent.

432. The device of item 275, further comprising a polymer.

433. The device of item 275, further comprising a polymeric carrier.

434. The device of item 275 wherein the anti-scarring agent inhibits adhesion between the device and a host into which the device is implanted.

435. The device of item 275 wherein the device delivers the anti-scarring agent locally to tissue proximate to the device.

436. The device of item 275, further comprising a coating, wherein the coating comprises the anti-scarring agent.

437. The device of item 275, further comprising a coating, wherein the coating is disposed on a surface of the device.

438. The device of item 275, further comprising a coating, wherein the coating directly contacts the device.

439. The device of item 275, further comprising a coating, wherein the coating indirectly contacts the device.

440. The device of item 275, further comprising a coating, wherein the coating partially covers the device.

441. The device of item 275, further comprising a coating, wherein the coating completely covers the device.

442. The device of item 275, further comprising a coating, wherein the coating is a uniform coating.

443. The device of item 275, further comprising a coating, wherein the coating is a non-uniform coating.

444. The device of item 275, further comprising a coating, wherein the coating is a discontinuous coating.

445. The device of item 275, further comprising a coating, wherein the coating is a patterned coating.

446. The device of item 275, further comprising a coating, wherein the coating has a thickness of 100 μm or less.

447. The device of item 275, further comprising a coating, wherein the coating has a thickness of 10 μm or less.

448. The device of item 275, further comprising a coating, wherein the coating adheres to the surface of the device upon deployment of the device.

449. The device of item 275, further comprising a coating, wherein the coating is stable at room temperature for a period of 1 year.

450. The device of item 275, further comprising a coating, wherein the anti-scarring agent is present in the coating in an amount ranging between about 0.0001% to about 1% by weight.

451. The device of item 275, further comprising a coating, wherein the anti-scarring agent is present in the coating in an amount ranging between about 1% to about 10% by weight.

452. The device of item 275, further comprising a coating, wherein the anti-scarring agent is present in the coating in an amount ranging between about 10% to about 25% by weight.

453. The device of item 275, further comprising a coating, wherein the anti-scarring agent is present in the coating in an amount ranging between about 25% to about 70% by weight.

454. The device of item 275, further comprising a coating, wherein the coating further comprises a polymer.

455. The device of item 275, further comprising a first coating having a first composition and the second coating having a second composition.

456. The device of item 275, further comprising a first coating having a first composition and the second coating having a second composition, wherein the first composition and the second composition are different.

457. The device of item 275, further comprising a polymer.

458. The device of item 275, further comprising a polymeric carrier.

459. The device of item 275, further comprising a polymeric carrier, wherein the polymeric carrier comprises a copolymer.

460. The device of item 275, further comprising a polymeric carrier, wherein the polymeric carrier comprises a block copolymer.

461. The device of item 275, further comprising a polymeric carrier, wherein the polymeric carrier comprises a random copolymer.

462. The device of item 275, further comprising a polymeric carrier, wherein the polymeric carrier comprises a biodegradable polymer.

463. The device of item 275, further comprising a polymeric carrier, wherein the polymeric carrier comprises a non-biodegradable polymer.

464. The device of item 275, further comprising a polymeric carrier, wherein the polymeric carrier comprises a hydrophilic polymer.

465. The device of item 275, further comprising a polymeric carrier, wherein the polymeric carrier comprises a hydrophobic polymer.

466. The device of item 275, further comprising a polymeric carrier, wherein the polymeric carrier comprises a polymer having hydrophilic domains.

467. The device of item 275, further comprising a polymeric carrier, wherein the polymeric carrier comprises a polymer having hydrophobic domains.

468. The device of item 275, further comprising a polymeric carrier, wherein the polymeric carrier comprises a non-conductive polymer.

469. The device of item 275, further comprising a polymeric carrier, wherein the polymeric carrier comprises an elastomer.

470. The device of item 275, further comprising a polymeric carrier, wherein the polymeric carrier comprises a hydrogel.

471. The device of item 275, further comprising a polymeric carrier, wherein the polymeric carrier comprises a silicone polymer.

472. The device of item 275, further comprising a polymeric carrier, wherein the polymeric carrier comprises a hydrocarbon polymer.

473. The device of item 275, further comprising a polymeric carrier, wherein the polymeric carrier comprises a styrene-derived polymer.

474. The device of item 275, further comprising a polymeric carrier, wherein the polymeric carrier comprises a butadiene polymer.

475. The device of item 275, further comprising a polymeric carrier, wherein the polymeric carrier comprises a macromer.

476. The device of item 275, further comprising a polymeric carrier, wherein the polymeric carrier comprises a poly(ethylene glycol)polymer.

477. The device of item 275, further comprising a polymeric carrier, wherein the polymeric carrier comprises an amorphous polymer.

478. The device of item 275, further comprising a lubricious coating.

479. The device of item 275 wherein the anti-scarring agent is located within pores or holes of the device.

480. The device of item 275 wherein the anti-scarring agent is located within a channel, lumen, or divet of the device.

481. The device of item 275, further comprising a second pharmaceutically active agent.

482. The device of item 275, further comprising an anti-inflammatory agent.

483. The device of item 275, further comprising an agent that inhibits infection.

484. The device of item 275, further comprising an agent that inhibits infection, wherein the agent is an anthracycline.

485. The device of item 275, further comprising an agent that inhibits infection, wherein the agent is doxorubicin.

486. The device of item 275, further comprising an agent that inhibits infection, wherein the agent is mitoxantrone.

487. The device of item 275, further comprising an agent that inhibits infection, wherein the agent is a fluoropyrimidine.

488. The device of item 275, further comprising an agent that inhibits infection, wherein the agent is 5-fluorouracil (5-FU).

489. The device of item 275, further comprising an agent that inhibits infection, wherein the agent is a folic acid antagonist.

490. The device of item 275, further comprising an agent that inhibits infection, wherein the agent is methotrexate.

491. The device of item 275, further comprising an agent that inhibits infection, wherein the agent is a podophylotoxin.

492. The device of item 275, further comprising an agent that inhibits infection, wherein the agent is etoposide.

493. The device of item 275, further comprising an agent that inhibits infection, wherein the agent is a camptothecin.

494. The device of item 275, further comprising an agent that inhibits infection, wherein the agent is a hydroxyurea.

495. The device of item 275, further comprising an agent that inhibits infection, wherein the agent is a platinum complex.

496. The device of item 275, further comprising an agent that inhibits infection, wherein the agent is cisplatin.

497. The device of item 275, further comprising an anti-thrombotic agent.

498. The device of item 275, further comprising a visualization agent.

499. The device of item 275, further comprising a visualization agent, wherein the visualization agent is a radiopaque material, wherein the radiopaque material comprises a metal, a halogenated compound, or a barium containing compound.

500. The device of item 275, further comprising a visualization agent, wherein the visualization agent is a radiopaque material, wherein the radiopaque material comprises barium, tantalum, or technetium.

501. The device of item 275, further comprising a visualization agent, wherein the visualization agent is a MRI responsive material.

502. The device of item 275, further comprising a visualization agent, wherein the visualization agent comprises a gadolinium chelate.

503. The device of item 275, further comprising a visualization agent, wherein the visualization agent comprises iron, magnesium, manganese, copper, or chromium.

504. The device of item 275, further comprising a visualization agent, wherein the visualization agent comprises an iron oxide compound.

505. The device of item 275, further comprising a visualization agent, wherein the visualization agent comprises a dye, pigment, or colorant.

506. The device of item 275, further comprising an echogenic material.

507. The device of item 275, further comprising an echogenic material, wherein the echogenic material is in the form of a coating.

508. The device of item 275 wherein the device is sterile.

509. The device of item 275 wherein the anti-scarring agent is released into tissue in the vicinity of the device after deployment of the device.

510. The device of item 275 wherein the anti-scarring agent is released into tissue in the vicinity of the device after deployment of the device, wherein the tissue is connective tissue.

511. The device of item 275 wherein the anti-scarring agent is released into tissue in the vicinity of the device after deployment of the device, wherein the tissue is muscle tissue.

512. The device of item 275 wherein the anti-scarring agent is released into tissue in the vicinity of the device after deployment of the device, wherein the tissue is nerve tissue.

513. The device of item 275 wherein the anti-scarring agent is released into tissue in the vicinity of the device after deployment of the device, wherein the tissue is epithelium tissue.

514. The device of item 275 wherein the anti-scarring agent is released in effective concentrations from the device over a period ranging from the time of deployment of the device to about 1 year.

515. The device of item 275 wherein the anti-scarring agent is released in effective concentrations from the device over a period ranging from about 1 month to 6 months.

516. The device of item 275 wherein the anti-scarring agent is released in effective concentrations from the device over a period ranging from about 1-90 days.

517. The device of item 275 wherein the anti-scarring agent is released in effective concentrations from the device at a constant rate.

518. The device of item 275 wherein the anti-scarring agent is released in effective concentrations from the device at an increasing rate.

519. The device of item 275 wherein the anti-scarring agent is released in effective concentrations from the device at a decreasing rate.

520. The device of item 275 wherein the anti-scarring agent is released in effective concentrations from the composition comprising the anti-scarring agent by diffusion over a period ranging from the time of deployment of the device to about 90 days.

521. The device of item 275 wherein the anti-scarring agent is released in effective concentrations from the composition comprising the anti-scarring agent by erosion of the composition over a period ranging from the time of deployment of the device to about 90 days.

522. The device of item 275 wherein the device comprises about 0.01 μg to about 10 μg of the anti-scarring agent.

523. The device of item 275 wherein the device comprises about 10 μg to about 10 mg of the anti-scarring agent.

524. The device of item 275 wherein the device comprises about 10 mg to about 250 mg of the anti-scarring agent.

525. The device of item 275 wherein the device comprises about 250 mg to about 1000 mg of the anti-scarring agent.

526. The device of item 275 wherein the device comprises about 1000 mg to about 2500 mg of the anti-scarring agent.

527. The device of item 275 wherein a surface of the device comprises less than 0.01 μg of the anti-scarring agent per mm2 of device surface to which the anti-scarring agent is applied.

528. The device of item 275 wherein a surface of the device comprises about 0.01 μg to about 1 μg of the anti-scarring agent per mm2 of device surface to which the anti-scarring agent is applied.

529. The device of item 275 wherein a surface of the device comprises about 1 μg to about 10 μg of the anti-scarring agent per mm2 of device surface to which the anti-scarring agent is applied.

530. The device of item 275 wherein a surface of the device comprises about 10 μg to about 250 μg of the anti-scarring agent per mm2 of device surface to which the anti-scarring agent is applied.

531. The device of item 275 wherein a surface of the device comprises about 250 μg to about 1000 μg of the anti-scarring agent of anti-scarring agent per mm2 of device surface to which the anti-scarring agent is applied.

532. The device of item 275 wherein a surface of the device comprises about 1000 μg to about 2500 μg of the anti-scarring agent per mm2 of device surface to which the anti-scarring agent is applied.

533. The device of any one of items 275-532 wherein the implant is a synthetic bypass graft.

534. The device of any one of items 275-532 wherein the implant is a femoral-popliteal synthetic bypass graft.

535. The device of any one of items 275-532 wherein the implant is a femoral-femoral synthetic bypass graft.

536. The device of any one of items 275-532 wherein the implant is an axillary-femoral synthetic bypass graft.

537. The device of any one of items 275-532 wherein the implant is a vein graft.

538. The device of any one of items 275-532 wherein the implant is a peripheral vein graft.

539. The device of any one of items 275-532 wherein the implant is a coronary vein graft.

540. The device of any one of items 275-532 wherein the implant is an internal mammary graft.

541. The device of any one of items 275-532 wherein the implant is an internal mammary coronary graft.

542. The device of any one of items 275-532 wherein the implant is a bifurcated vascular graft.

543. The device of any one of items 275-532 wherein the implant is a vascular wrap.

544. A device, comprising an implant for hemodialysis access (i.e., a hemodialysis access device) and an anti-scarring agent or a composition comprising an anti-scarring agent, wherein the agent inhibits scarring between the device and a host into which the device is implanted.

545. The device of item 544 wherein the agent inhibits cell regeneration.

546. The device of item 544 wherein the agent inhibits angiogenesis.

547. The device of item 544 wherein the agent inhibits fibroblast migration.

548. The device of item 544 wherein the agent inhibits fibroblast proliferation.

549. The device of item 544 wherein the agent inhibits deposition of extracellular matrix.

550. The device of item 544 wherein the agent inhibits tissue remodeling.

551. The device of item 544 wherein the agent is an angiogenesis inhibitor.

552. The device of item 544 wherein the agent is a 5-lipoxygenase inhibitor or antagonist.

553. The device of item 544 wherein the agent is a chemokine receptor antagonist.

554. The device of item 544 wherein the agent is a cell cycle inhibitor.

555. The device of item 544 wherein the agent is a taxane.

556. The device of item 544 wherein the agent is an anti-microtubule agent.

557. The device of item 544 wherein the agent is paclitaxel.

558. The device of item 544 wherein the agent is not paclitaxel.

559. The device of item 544 wherein the agent is an analogue or derivative of paclitaxel.

560. The device of item 544 wherein the agent is a vinca alkaloid.

561. The device of item 544 wherein the agent is camptothecin or an analogue or derivative thereof.

562. The device of item 544 wherein the agent is a podophyllotoxin.

563. The device of item 544 wherein the agent is a podophyllotoxin, wherein the podophyllotoxin is etoposide or an analogue or derivative thereof.

564. The device of item 544 wherein the agent is an anthracycline.

565. The device of item 544 wherein the agent is an anthracycline, wherein the anthracycline is doxorubicin or an analogue or derivative thereof.

566. The device of item 544 wherein the agent is an anthracycline, wherein the anthracycline is mitoxantrone or an analogue or derivative thereof.

567. The device of item 544 wherein the agent is a platinum compound.

568. The device of item 544 wherein the agent is a nitrosourea.

569. The device of item 544 wherein the agent is a nitroimidazole.

570. The device of item 544 wherein the agent is a folic acid antagonist.

571. The device of item 544 wherein the agent is a cytidine analogue.

572. The device of item 544 wherein the agent is a pyrimidine analogue.

573. The device of item 544 wherein the agent is a fluoropyrimidine analogue.

574. The device of item 544 wherein the agent is a purine analogue.

575. The device of item 544 wherein the agent is a nitrogen mustard or an analogue or derivative thereof.

576. The device of item 544 wherein the agent is a hydroxyurea.

577. The device of item 544 wherein the agent is a mytomicin or an analogue or derivative thereof.

578. The device of item 544 wherein the agent is an alkyl sulfonate.

579. The device of item 544 wherein the agent is a benzamide or an analogue or derivative thereof.

580. The device of item 544 wherein the agent is a nicotinamide or an analogue or derivative thereof.

581. The device of item 544 wherein the agent is a halogenated sugar or an analogue or derivative thereof.

582. The device of item 544 wherein the agent is a DNA alkylating agent.

583. The device of item 544 wherein the agent is an anti-microtubule agent.

584. The device of item 544 wherein the agent is a topoisomerase inhibitor.

585. The device of item 544 wherein the agent is a DNA cleaving agent.

586. The device of item 544 wherein the agent is an antimetabolite.

587. The device of item 544 wherein the agent inhibits adenosine deaminase.

588. The device of item 544 wherein the agent inhibits purine ring synthesis.

589. The device of item 544 wherein the agent is a nucleotide interconversion inhibitor.

590. The device of item 544 wherein the agent inhibits dihydrofolate reduction.

591. The device of item 544 wherein the agent blocks thymidine mono phosphate.

592. The device of item 544 wherein the agent causes DNA damage.

593. The device of item 544 wherein the agent is a DNA intercalation agent.

594. The device of item 544 wherein the agent is a RNA synthesis inhibitor.

595. The device of item 544 wherein the agent is a pyrimidine synthesis inhibitor.

596. The device of item 544 wherein the agent inhibits ribonucleotide synthesis or function.

597. The device of item 544 wherein the agent inhibits thymidine monophosphate synthesis or function.

598. The device of item 544 wherein the agent inhibits DNA synthesis.

599. The device of item 544 wherein the agent causes DNA adduct formation.

600. The device of item 544 wherein the agent inhibits protein synthesis.

601. The device of item 544 wherein the agent inhibits microtubule function.

602. The device of item 544 wherein the agent is a cyclin dependent protein kinase inhibitor.

603. The device of item 544 wherein the agent is an epidermal growth factor kinase inhibitor.

604. The device of item 544 wherein the agent is an elastase inhibitor.

605. The device of item 544 wherein the agent is a factor Xa inhibitor.

606. The device of item 544 wherein the agent is a farnesyltransferase inhibitor.

607. The device of item 544 wherein the agent is a fibrinogen antagonist.

608. The device of item 544 wherein the agent is a guanylate cyclase stimulant.

609. The device of item 544 wherein the agent is a heat shock protein 90 antagonist.

610. The device of item 544 wherein the agent is a heat shock protein 90 antagonist, wherein the heat shock protein 90 antagonist is geldanamycin or an analogue or derivative thereof.

611. The device of item 544 wherein the agent is a guanylate cyclase stimulant.

612. The device of item 544 wherein the agent is a HMGCoA reductase inhibitor.

613. The device of item 544 wherein the agent is a HMGCoA reductase inhibitor, wherein the HMGCoA reductase inhibitor is simvastatin or an analogue or derivative thereof.

614. The device of item 544 wherein the agent is a hydroorotate dehydrogenase inhibitor.

615. The device of item 544 wherein the agent is an IKK2 inhibitor.

616. The device of item 544 wherein the agent is an IL-1 antagonist.

617. The device of item 544 wherein the agent is an ICE antagonist.

618. The device of item 544 wherein the agent is an IRAK antagonist.

619. The device of item 544 wherein the agent is an IL-4 agonist.

620. The device of item 544 wherein the agent is an immunomodulatory agent.

621. The device of item 544 wherein the agent is sirolimus or an analogue or derivative thereof.

622. The device of item 544 wherein the agent is not sirolimus.

623. The device of item 544 wherein the agent is everolimus or an analogue or derivative thereof.

624. The device of item 544 wherein the agent is tacrolimus or an analogue or derivative thereof.

625. The device of item 544 wherein the agent is not tacrolimus.

626. The device of item 544 wherein the agent is biolmus or an analogue or derivative thereof.

627. The device of item 544 wherein the agent is tresperimus or an analogue or derivative thereof.

628. The device of item 544 wherein the agent is auranofin or an analogue or derivative thereof.

629. The device of item 544 wherein the agent is 27-0-demethylrapamycin or an analogue or derivative thereof.

630. The device of item 544 wherein the agent is gusperimus or an analogue or derivative thereof.

631. The device of item 544 wherein the agent is pimecrolimus or an analogue or derivative thereof.

632. The device of item 544 wherein the agent is ABT-578 or an analogue or derivative thereof.

633. The device of item 544 wherein the agent is an inosine monophosphate dehydrogenase (IMPDH) inhibitor.

634. The device of item 544 wherein the agent is an IMPDH inhibitor, wherein the IMPDH inhibitor is mycophenolic acid or an analogue or derivative thereof.

635. The device of item 544 wherein the agent is an IMPDH inhibitor, wherein the IMPDH inhibitor is 1-alpha-25 dihydroxy vitamin D3 or an analogue or derivative thereof.

636. The device of item 544 wherein the agent is a leukotriene inhibitor.

637. The device of item 544 wherein the agent is a MCP-1 antagonist.

638. The device of item 544 wherein the agent is a MMP inhibitor.

639. The device of item 544 wherein the agent is an NF kappa B inhibitor.

640. The device of item 544 wherein the agent is an NF kappa B inhibitor, wherein the NF kappa B inhibitor is Bay 11-7082.

641. The device of item 544 wherein the agent is an NO agonist.

642. The device of item 544 wherein the agent is a p38 MAP kinase inhibitor.

643. The device of item 544 wherein the agent is a p38 MAP kinase inhibitor, wherein the p38 MAP kinase inhibitor is SB 202190.

644. The device of item 544 wherein the agent is a phosphodiesterase inhibitor.

645. The device of item 544 wherein the agent is a TGF beta inhibitor.

646. The device of item 544 wherein the agent is a thromboxane A2 antagonist.

647. The device of item 544 wherein the agent is a TNFa antagonist.

648. The device of item 544 wherein the agent is a TACE inhibitor.

649. The device of item 544 wherein the agent is a tyrosine kinase inhibitor.

650. The device of item 544 wherein the agent is a vitronectin inhibitor.

651. The device of item 544 wherein the agent is a fibroblast growth factor inhibitor.

652. The device of item 544 wherein the agent is a protein kinase inhibitor.

653. The device of item 544 wherein the agent is a PDGF receptor kinase inhibitor.

654. The device of item 544 wherein the agent is an endothelial growth factor receptor kinase inhibitor.

655. The device of item 544 wherein the agent is a retinoic acid receptor antagonist.

656. The device of item 544 wherein the agent is a platelet derived growth factor receptor kinase inhibitor.

657. The device of item 544 wherein the agent is a fibronogin antagonist.

658. The device of item 544 wherein the agent is an antimycotic agent.

659. The device of item 544 wherein the agent is an antimycotic agent, wherein the antimycotic agent is sulconizole.

660. The device of item 544 wherein the agent is a bisphosphonate.

661. The device of item 544 wherein the agent is a phospholipase A1 inhibitor.

662. The device of item 544 wherein the agent is a histamine H1/H2/H3 receptor antagonist.

663. The device of item 544 wherein the agent is a macrolide antibiotic.

664. The device of item 544 wherein the agent is a GPIIb/IIIa receptor antagonist.

665. The device of item 544 wherein the agent is an endothelin receptor antagonist.

666. The device of item 544 wherein the agent is a peroxisome proliferator-activated receptor agonist.

667. The device of item 544 wherein the agent is an estrogen receptor agent.

668. The device of item 544 wherein the agent is a somastostatin analogue.

669. The device of item 544 wherein the agent is a neurokinin 1 antagonist.

670. The device of item 544 wherein the agent is a neurokinin 3 antagonist.

671. The device of item 544 wherein the agent is a VLA-4 antagonist.

672. The device of item 544 wherein the agent is an osteoclast inhibitor.

673. The device of item 544 wherein the agent is a DNA topoisomerase ATP hydrolyzing inhibitor.

674. The device of item 544 wherein the agent is an angiotensin I converting enzyme inhibitor.

675. The device of item 544 wherein the agent is an angiotensin II antagonist.

676. The device of item 544 wherein the agent is an enkephalinase inhibitor.

677. The device of item 544 wherein the agent is a peroxisome proliferator-activated receptor gamma agonist insulin sensitizer.

678. The device of item 544 wherein the agent is a protein kinase C inhibitor.

679. The device of item 544 wherein the agent is a ROCK (rho-associated kinase) inhibitor.

680. The device of item 544 wherein the agent is a CXCR3 inhibitor.

681. The device of item 544 wherein the agent is an Itk inhibitor.

682. The device of item 544 wherein the agent is a cytosolic phospholipase A2-alpha inhibitor.

683. The device of item 544 wherein the agent is a PPAR agonist.

684. The device of item 544 wherein the agent is an immunosuppressant.

685. The device of item 544 wherein the agent is an Erb inhibitor.

686. The device of item 544 wherein the agent is an apoptosis agonist.

687. The device of item 544 wherein the agent is a lipocortin agonist.

688. The device of item 544 wherein the agent is a VCAM-1 antagonist.

689. The device of item 544 wherein the agent is a collagen antagonist.

690. The device of item 544 wherein the agent is an alpha 2 integrin antagonist.

691. The device of item 544 wherein the agent is a TNF alpha inhibitor.

692. The device of item 544 wherein the agent is a nitric oxide inhibitor.

693. The device of item 544 wherein the agent is a cathepsin inhibitor.

694. The device of item 544 wherein the agent is not an anti-inflammatory agent.

695. The device of item 544 wherein the agent is not a steroid.

696. The device of item 544 wherein the agent is not a glucocorticosteroid.

697. The device of item 544 wherein the agent is not dexamethasone.

698. The device of item 544 wherein the agent is not an anti-infective agent.

699. The device of item 544 wherein the agent is not an antibiotic.

700. The device of item 544 wherein the agent is not an anti-fungal agent.

701. The device of item 544, further comprising a polymer.

702. The device of item 544, further comprising a polymeric carrier.

703. The device of item 544 wherein the anti-scarring agent inhibits adhesion between the device and a host into which the device is implanted.

704. The device of item 544 wherein the device delivers the anti-scarring agent locally to tissue proximate to the device.

705. The device of item 544, further comprising a coating, wherein the coating comprises the anti-scarring agent.

706. The device of item 544, further comprising a coating, wherein the coating is disposed on a surface of the device.

707. The device of item 544, further comprising a coating, wherein the coating directly contacts the device.

708. The device of item 544, further comprising a coating, wherein the coating indirectly contacts the device.

709. The device of item 544, further comprising a coating, wherein the coating partially covers the device.

710. The device of item 544, further comprising a coating, wherein the coating completely covers the device.

711. The device of item 544, further comprising a coating, wherein the coating is a uniform coating.

712. The device of item 544, further comprising a coating, wherein the coating is a non-uniform coating.

713. The device of item 544, further comprising a coating, wherein the coating is a discontinuous coating.

714. The device of item 544, further comprising a coating, wherein the coating is a patterned coating.

715. The device of item 544, further comprising a coating, wherein the coating has a thickness of 100 μm or less.

716. The device of item 544, further comprising a coating, wherein the coating has a thickness of 10 μm or less.

717. The device of item 544, further comprising a coating, wherein the coating adheres to the surface of the device upon deployment of the device.

718. The device of item 544, further comprising a coating, wherein the coating is stable at room temperature for a period of 1 year.

719. The device of item 544, further comprising a coating, wherein the anti-scarring agent is present in the coating in an amount ranging between about 0.0001% to about 1% by weight.

720. The device of item 544, further comprising a coating, wherein the anti-scarring agent is present in the coating in an amount ranging between about 1% to about 10% by weight.

721. The device of item 544, further comprising a coating, wherein the anti-scarring agent is present in the coating in an amount ranging between about 10% to about 25% by weight.

722. The device of item 544, further comprising a coating, wherein the anti-scarring agent is present in the coating in an amount ranging between about 25% to about 70% by weight.

723. The device of item 544, further comprising a coating, wherein the coating further comprises a polymer.

724. The device of item 544, further comprising a first coating having a first composition and the second coating having a second composition.

725. The device of item 544, further comprising a first coating having a first composition and the second coating having a second composition, wherein the first composition and the second composition are different.

726. The device of item 544, further comprising a polymer.

727. The device of item 544, further comprising a polymeric carrier.

728. The device of item 544, further comprising a polymeric carrier, wherein the polymeric carrier comprises a copolymer.

729. The device of item 544, further comprising a polymeric carrier, wherein the polymeric carrier comprises a block copolymer.

730. The device of item 544, further comprising a polymeric carrier, wherein the polymeric carrier comprises a random copolymer.

731. The device of item 544, further comprising a polymeric carrier, wherein the polymeric carrier comprises a biodegradable polymer.

732. The device of item 544, further comprising a polymeric carrier, wherein the polymeric carrier comprises a non-biodegradable polymer.

733. The device of item 544, further comprising a polymeric carrier, wherein the polymeric carrier comprises a hydrophilic polymer.

734. The device of item 544, further comprising a polymeric carrier, wherein the polymeric carrier comprises a hydrophobic polymer.

735. The device of item 544, further comprising a polymeric carrier, wherein the polymeric carrier comprises a polymer having hydrophilic domains.

736. The device of item 544, further comprising a polymeric carrier, wherein the polymeric carrier comprises a polymer having hydrophobic domains.

737. The device of item 544, further comprising a polymeric carrier, wherein the polymeric carrier comprises a non-conductive polymer.

738. The device of item 544, further comprising a polymeric carrier, wherein the polymeric carrier comprises an elastomer.

739. The device of item 544, further comprising a polymeric carrier, wherein the polymeric carrier comprises a hydrogel.

740. The device of item 544, further comprising a polymeric carrier, wherein the polymeric carrier comprises a silicone polymer.

741. The device of item 544, further comprising a polymeric carrier, wherein the polymeric carrier comprises a hydrocarbon polymer.

742. The device of item 544, further comprising a polymeric carrier, wherein the polymeric carrier comprises a styrene-derived polymer.

743. The device of item 544, further comprising a polymeric carrier, wherein the polymeric carrier comprises a butadiene polymer.

744. The device of item 544, further comprising a polymeric carrier, wherein the polymeric carrier comprises a macromer.

745. The device of item 544, further comprising a polymeric carrier, wherein the polymeric carrier comprises a poly(ethylene glycol)polymer.

746. The device of item 544, further comprising a polymeric carrier, wherein the polymeric carrier comprises an amorphous polymer.

747. The device of item 544, further comprising a lubricious coating.

748. The device of item 544 wherein the anti-scarring agent is located within pores or holes of the device.

749. The device of item 544 wherein the anti-scarring agent is located within a channel, lumen, or divet of the device.

750. The device of item 544, further comprising a second pharmaceutically active agent.

751. The device of item 544, further comprising an anti-inflammatory agent.

752. The device of item 544, further comprising an agent that inhibits infection.

753. The device of item 544, further comprising an agent that inhibits infection, wherein the agent is an anthracycline.

754. The device of item 544, further comprising an agent that inhibits infection, wherein the agent is doxorubicin.

755. The device of item 544, further comprising an agent that inhibits infection, wherein the agent is mitoxantrone.

756. The device of item 544, further comprising an agent that inhibits infection, wherein the agent is a fluoropyrimidine.

757. The device of item 544, further comprising an agent that inhibits infection, wherein the agent is 5-fluorouracil (5-FU).

758. The device of item 544, further comprising an agent that inhibits infection, wherein the agent is a folic acid antagonist.

759. The device of item 544, further comprising an agent that inhibits infection, wherein the agent is methotrexate.

760. The device of item 544, further comprising an agent that inhibits infection, wherein the agent is a podophylotoxin.

761. The device of item 544, further comprising an agent that inhibits infection, wherein the agent is etoposide.

762. The device of item 544, further comprising an agent that inhibits infection, wherein the agent is a camptothecin.

763. The device of item 544, further comprising an agent that inhibits infection, wherein the agent is a hydroxyurea.

764. The device of item 544, further comprising an agent that inhibits infection, wherein the agent is a platinum complex.

765. The device of item 544, further comprising an agent that inhibits infection, wherein the agent is cisplatin.

766. The device of item 544, further comprising an anti-thrombotic agent.

767. The device of item 544, further comprising a visualization agent.

768. The device of item 544, further comprising a visualization agent, wherein the visualization agent is a radiopaque material, wherein the radiopaque material comprises a metal, a halogenated compound, or a barium containing compound.

769. The device of item 544, further comprising a visualization agent, wherein the visualization agent is a radiopaque material, wherein the radiopaque material comprises barium, tantalum, or technetium.

770. The device of item 544, further comprising a visualization agent, wherein the visualization agent is a MRI responsive material.

771. The device of item 544, further comprising a visualization agent, wherein the visualization agent comprises a gadolinium chelate.

772. The device of item 544, further comprising a visualization agent, wherein the visualization agent comprises iron, magnesium, manganese, copper, or chromium.

773. The device of item 544, further comprising a visualization agent, wherein the visualization agent comprises an iron oxide compound.

774. The device of item 544, further comprising a visualization agent, wherein the visualization agent comprises a dye, pigment, or colorant.

775. The device of item 544, further comprising an echogenic material.

776. The device of item 544, further comprising an echogenic material, wherein the echogenic material is in the form of a coating.

777. The device of item 544 wherein the device is sterile.

778. The device of item 544 wherein the anti-scarring agent is released into tissue in the vicinity of the device after deployment of the device.

779. The device of item 544 wherein the anti-scarring agent is released into tissue in the vicinity of the device after deployment of the device, wherein the tissue is connective tissue.

780. The device of item 544 wherein the anti-scarring agent is released into tissue in the vicinity of the device after deployment of the device, wherein the tissue is muscle tissue.

781. The device of item 544 wherein the anti-scarring agent is released into tissue in the vicinity of the device after deployment of the device, wherein the tissue is nerve tissue.

782. The device of item 544 wherein the anti-scarring agent is released into tissue in the vicinity of the device after deployment of the device, wherein the tissue is epithelium tissue.

783. The device of item 544 wherein the anti-scarring agent is released in effective concentrations from the device over a period ranging from the time of deployment of the device to about 1 year.

784. The device of item 544 wherein the anti-scarring agent is released in effective concentrations from the device over a period ranging from about 1 month to 6 months.

785. The device of item 544 wherein the anti-scarring agent is released in effective concentrations from the device over a period ranging from about 1-90 days.

786. The device of item 544 wherein the anti-scarring agent is released in effective concentrations from the device at a constant rate.

787. The device of item 544 wherein the anti-scarring agent is released in effective concentrations from the device at an increasing rate.

788. The device of item 544 wherein the anti-scarring agent is released in effective concentrations from the device at a decreasing rate.

789. The device of item 544 wherein the anti-scarring agent is released in effective concentrations from the composition comprising the anti-scarring agent by diffusion over a period ranging from the time of deployment of the device to about 90 days.

790. The device of item 544 wherein the anti-scarring agent is released in effective concentrations from the composition comprising the anti-scarring agent by erosion of the composition over a period ranging from the time of deployment of the device to about 90 days.

791. The device of item 544 wherein the device comprises about 0.01 μg to about 10 μg of the anti-scarring agent.

792. The device of item 544 wherein the device comprises about 10 μg to about 10 mg of the anti-scarring agent.

793. The device of item 544 wherein the device comprises about 10 mg to about 250 mg of the anti-scarring agent.

794. The device of item 544 wherein the device comprises about 250 mg to about 1000 mg of the anti-scarring agent.

795. The device of item 544 wherein the device comprises about 1000 mg to about 2500 mg of the anti-scarring agent.

796. The device of item 544 wherein a surface of the device comprises less than 0.01 μg of the anti-scarring agent per mm2 of device surface to which the anti-scarring agent is applied.

797. The device of item 544 wherein a surface of the device comprises about 0.01 μg to about 1 μg of the anti-scarring agent per mm2 of device surface to which the anti-scarring agent is applied.

798. The device of item 544 wherein a surface of the device comprises about 1 μg to about 10 μg of the anti-scarring agent per mm2 of device surface to which the anti-scarring agent is applied.

799. The device of item 544 wherein a surface of the device comprises about 10 μg to about 250 μg of the anti-scarring agent per mm2 of device surface to which the anti-scarring agent is applied.

800. The device of item 544 wherein a surface of the device comprises about 250 μg to about 1000 μg of the anti-scarring agent of anti-scarring agent per mm2 of device surface to which the anti-scarring agent is applied.

801. The device of item 544 wherein a surface of the device comprises about 1000 μg to about 2500 μg of the anti-scarring agent per mm2 of device surface to which the anti-scarring agent is applied.

802. The device of any one of items 544-801 wherein the implant is an AV fistula.

803. The device of any one of items 544-801 wherein the implant is an AV access graft.

804. The device of any one of items 544-801 wherein the implant is a venous catheter.

805. The device of any one of items 544-801 wherein the implant is an implantable port.

806. The device of any one of items 544-801 wherein the implant is an AV shunt.

807. A device, comprising an implant that provides an anastomotic connection (i.e., an anastomotic connector device) and an anti-scarring agent or a composition comprising an anti-scarring agent, wherein the agent inhibits scarring between the device and a host into which the device is implanted.

808. The device of item 807 wherein the agent inhibits cell regeneration.

809. The device of item 807 wherein the agent inhibits angiogenesis.

810. The device of item 807 wherein the agent inhibits fibroblast migration.

811. The device of item 807 wherein the agent inhibits fibroblast proliferation.

812. The device of item 807 wherein the agent inhibits deposition of extracellular matrix.

813. The device of item 807 wherein the agent inhibits tissue remodeling.

814. The device of item 807 wherein the agent is an angiogenesis inhibitor.

815. The device of item 807 wherein the agent is a 5-lipoxygenase inhibitor or antagonist.

816. The device of item 807 wherein the agent is a chemokine receptor antagonist.

817. The device of item 807 wherein the agent is a cell cycle inhibitor.

818. The device of item 807 wherein the agent is a taxane.

819. The device of item 807 wherein the agent is an anti-microtubule agent.

820. The device of item 807 wherein the agent is paclitaxel.

821. The device of item 807 wherein the agent is not paclitaxel.

822. The device of item 807 wherein the agent is an analogue or derivative of paclitaxel.

823. The device of item 807 wherein the agent is a vinca alkaloid.

824. The device of item 807 wherein the agent is camptothecin or an analogue or derivative thereof.

825. The device of item 807 wherein the agent is a podophyllotoxin.

826. The device of item 807 wherein the agent is a podophyllotoxin, wherein the podophyllotoxin is etoposide or an analogue or derivative thereof.

827. The device of item 807 wherein the agent is an anthracycline.

828. The device of item 807 wherein the agent is an anthracycline, wherein the anthracycline is doxorubicin or an analogue or derivative thereof.

829. The device of item 807 wherein the agent is an anthracycline, wherein the anthracycline is mitoxantrone or an analogue or derivative thereof.

830. The device of item 807 wherein the agent is a platinum compound.

831. The device of item 807 wherein the agent is a nitrosourea.

832. The device of item 807 wherein the agent is a nitroimidazole.

833. The device of item 807 wherein the agent is a folic acid antagonist.

834. The device of item 807 wherein the agent is a cytidine analogue.

835. The device of item 807 wherein the agent is a pyrimidine analogue.

836. The device of item 807 wherein the agent is a fluoropyrimidine analogue.

837. The device of item 807 wherein the agent is a purine analogue.

838. The device of item 807 wherein the agent is a nitrogen mustard or an analogue or derivative thereof.

839. The device of item 807 wherein the agent is a hydroxyurea.

840. The device of item 807 wherein the agent is a mytomicin or an analogue or derivative thereof.

841. The device of item 807 wherein the agent is an alkyl sulfonate.

842. The device of item 807 wherein the agent is a benzamide or an analogue or derivative thereof.

843. The device of item 807 wherein the agent is a nicotinamide or an analogue or derivative thereof.

844. The device of item 807 wherein the agent is a halogenated sugar or an analogue or derivative thereof.

845. The device of item 807 wherein the agent is a DNA alkylating agent.

846. The device of item 807 wherein the agent is an anti-microtubule agent.

847. The device of item 807 wherein the agent is a topoisomerase inhibitor.

848. The device of item 807 wherein the agent is a DNA cleaving agent.

849. The device of item 807 wherein the agent is an antimetabolite.

850. The device of item 807 wherein the agent inhibits adenosine deaminase.

851. The device of item 807 wherein the agent inhibits purine ring synthesis.

852. The device of item 807 wherein the agent is a nucleotide interconversion inhibitor.

853. The device of item 807 wherein the agent inhibits dihydrofolate reduction.

854. The device of item 807 wherein the agent blocks thymidine monophosphate.

855. The device of item 807 wherein the agent causes DNA damage.

856. The device of item 807 wherein the agent is a DNA intercalation agent.

857. The device of item 807 wherein the agent is a RNA synthesis inhibitor.

858. The device of item 807 wherein the agent is a pyrimidine synthesis inhibitor.

859. The device of item 807 wherein the agent inhibits ribonucleotide synthesis or function.

860. The device of item 807 wherein the agent inhibits thymidine monophosphate synthesis or function.

861. The device of item 807 wherein the agent inhibits DNA synthesis.

862. The device of item 807 wherein the agent causes DNA adduct formation.

863. The device of item 807 wherein the agent inhibits protein synthesis.

864. The device of item 807 wherein the agent inhibits microtubule function.

865. The device of item 807 wherein the agent is a cyclin dependent protein kinase inhibitor.

866. The device of item 807 wherein the agent is an epidermal growth factor kinase inhibitor.

867. The device of item 807 wherein the agent is an elastase inhibitor.

868. The device of item 807 wherein the agent is a factor Xa inhibitor.

869. The device of item 807 wherein the agent is a farnesyltransferase inhibitor.

870. The device of item 807 wherein the agent is a fibrinogen antagonist.

871. The device of item 807 wherein the agent is a guanylate cyclase stimulant.

872. The device of item 807 wherein the agent is a heat shock protein 90 antagonist.

873. The device of item 807 wherein the agent is a heat shock protein 90 antagonist, wherein the heat shock protein 90 antagonist is geldanamycin or an analogue or derivative thereof.

874. The device of item 807 wherein the agent is a guanylate cyclase stimulant.

875. The device of item 807 wherein the agent is a HMGCoA reductase inhibitor.

876. The device of item 807 wherein the agent is a HMGCoA reductase inhibitor, wherein the HMGCoA reductase inhibitor is simvastatin or an analogue or derivative thereof.

877. The device of item 807 wherein the agent is a hydroorotate dehydrogenase inhibitor.

878. The device of item 807 wherein the agent is an IKK2 inhibitor.

879. The device of item 807 wherein the agent is an IL-1 antagonist.

880. The device of item 807 wherein the agent is an ICE antagonist.

881. The device of item 807 wherein the agent is an IRAK antagonist.

882. The device of item 807 wherein the agent is an IL-4 agonist.

883. The device of item 807 wherein the agent is an immunomodulatory agent.

884. The device of item 807 wherein the agent is sirolimus or an analogue or derivative thereof.

885. The device of item 807 wherein the agent is not sirolimus.

886. The device of item 807 wherein the agent is everolimus or an analogue or derivative thereof.

887. The device of item 807 wherein the agent is tacrolimus or an analogue or derivative thereof.

888. The device of item 807 wherein the agent is not tacrolimus.

889. The device of item 807 wherein the agent is biolmus or an analogue or derivative thereof.

890. The device of item 807 wherein the agent is tresperimus or an analogue or derivative thereof.

891. The device of item 807 wherein the agent is auranofin or an analogue or derivative thereof.

892. The device of item 807 wherein the agent is 27-0-demethylrapamycin or an analogue or derivative thereof.

893. The device of item 807 wherein the agent is gusperimus or an analogue or derivative thereof.

894. The device of item 807 wherein the agent is pimecrolimus or an analogue or derivative thereof.

895. The device of item 807 wherein the agent is ABT-578 or an analogue or derivative thereof.

896. The device of item 807 wherein the agent is an inosine monophosphate dehydrogenase (IMPDH) inhibitor.

897. The device of item 807 wherein the agent is an IMPDH inhibitor, wherein the IMPDH inhibitor is mycophenolic acid or an analogue or derivative thereof.

898. The device of item 807 wherein the agent is an IMPDH inhibitor, wherein the IMPDH inhibitor is 1-alpha-25 dihydroxy vitamin D3 or an analogue or derivative thereof.

899. The device of item 807 wherein the agent is a leukotriene inhibitor.

900. The device of item 807 wherein the agent is a MCP-1 antagonist.

901. The device of item 807 wherein the agent is a MMP inhibitor.

902. The device of item 807 wherein the agent is an NF kappa B inhibitor.

903. The device of item 807 wherein the agent is an NF kappa B inhibitor, wherein the NF kappa B inhibitor is Bay 11-7082.

904. The device of item 807 wherein the agent is an NO agonist.

905. The device of item 807 wherein the agent is a p38 MAP kinase inhibitor.

906. The device of item 807 wherein the agent is a p38 MAP kinase inhibitor, wherein the p38 MAP kinase inhibitor is SB 202190.

907. The device of item 807 wherein the agent is a phosphodiesterase inhibitor.

908. The device of item 807 wherein the agent is a TGF beta inhibitor.

909. The device of item 807 wherein the agent is a thromboxane A2 antagonist.

910. The device of item 807 wherein the agent is a TNFa antagonist.

911. The device of item 807 wherein the agent is a TACE inhibitor.

912. The device of item 807 wherein the agent is a tyrosine kinase inhibitor.

913. The device of item 807 wherein the agent is a vitronectin inhibitor.

914. The device of item 807 wherein the agent is a fibroblast growth factor inhibitor.

915. The device of item 807 wherein the agent is a protein kinase inhibitor.

916. The device of item 807 wherein the agent is a PDGF receptor kinase inhibitor.

917. The device of item 807 wherein the agent is an endothelial growth factor receptor kinase inhibitor.

918. The device of item 807 wherein the agent is a retinoic acid receptor antagonist.

919. The device of item 807 wherein the agent is a platelet derived growth factor receptor kinase inhibitor.

920. The device of item 807 wherein the agent is a fibronogin antagonist.

921. The device of item 807 wherein the agent is an antimycotic agent.

922. The device of item 807 wherein the agent is an antimycotic agent, wherein the antimycotic agent is sulconizole.

923. The device of item 807 wherein the agent is a bisphosphonate.

924. The device of item 807 wherein the agent is a phospholipase A1 inhibitor.

925. The device of item 807 wherein the agent is a histamine H1/H2/H3 receptor antagonist.

926. The device of item 807 wherein the agent is a macrolide antibiotic.

927. The device of item 807 wherein the agent is a GPIIb/IIIa receptor antagonist.

928. The device of item 807 wherein the agent is an endothelin receptor antagonist.

929. The device of item 807 wherein the agent is a peroxisome proliferator-activated receptor agonist.

930. The device of item 807 wherein the agent is an estrogen receptor agent.

931. The device of item 807 wherein the agent is a somastostatin analogue.

932. The device of item 807 wherein the agent is a neurokinin 1 antagonist.

933. The device of item 807 wherein the agent is a neurokinin 3 antagonist.

934. The device of item 807 wherein the agent is a VLA-4 antagonist.

935. The device of item 807 wherein the agent is an osteoclast inhibitor.

936. The device of item 807 wherein the agent is a DNA topoisomerase ATP hydrolyzing inhibitor.

937. The device of item 807 wherein the agent is an angiotensin I converting enzyme inhibitor.

938. The device of item 807 wherein the agent is an angiotensin II antagonist.

939. The device of item 807 wherein the agent is an enkephalinase inhibitor.

940. The device of item 807 wherein the agent is a peroxisome proliferator-activated receptor gamma agonist insulin sensitizer.

941. The device of item 807 wherein the agent is a protein kinase C inhibitor.

942. The device of item 807 wherein the agent is a ROCK (rho-associated kinase) inhibitor.

943. The device of item 807 wherein the agent is a CXCR3 inhibitor.

944. The device of item 807 wherein the agent is an Itk inhibitor.

945. The device of item 807 wherein the agent is a cytosolic phospholipase A2-alpha inhibitor.

946. The device of item 807 wherein the agent is a PPAR agonist.

947. The device of item 807 wherein the agent is an immunosuppressant.

948. The device of item 807 wherein the agent is an Erb inhibitor.

949. The device of item 807 wherein the agent is an apoptosis agonist.

950. The device of item 807 wherein the agent is a lipocortin agonist.

951. The device of item 807 wherein the agent is a VCAM-1 antagonist.

952. The device of item 807 wherein the agent is a collagen antagonist.

953. The device of item 807 wherein the agent is an alpha 2 integrin antagonist.

954. The device of item 807 wherein the agent is a TNF alpha inhibitor.

955. The device of item 807 wherein the agent is a nitric oxide inhibitor.

956. The device of item 807 wherein the agent is a cathepsin inhibitor.

957. The device of item 807 wherein the agent is not an anti-inflammatory agent.

958. The device of item 807 wherein the agent is not a steroid.

959. The device of item 807 wherein the agent is not a glucocorticosteroid.

960. The device of item 807 wherein the agent is not dexamethasone.

961. The device of item 807 wherein the agent is not an anti-infective agent.

962. The device of item 807 wherein the agent is not an antibiotic.

963. The device of item 807 wherein the agent is not an anti-fungal agent.

964. The device of item 807, further comprising a polymer.

965. The device of item 807, further comprising a polymeric carrier.

966. The device of item 807 wherein the anti-scarring agent inhibits adhesion between the device and a host into which the device is implanted.

967. The device of item 807 wherein the device delivers the anti-scarring agent locally to tissue proximate to the device.

968. The device of item 807, further comprising a coating, wherein the coating comprises the anti-scarring agent.

969. The device of item 807, further comprising a coating, wherein the coating is disposed on a surface of the device.

970. The device of item 807, further comprising a coating, wherein the coating directly contacts the device.

971. The device of item 807, further comprising a coating, wherein the coating indirectly contacts the device.

972. The device of item 807, further comprising a coating, wherein the coating partially covers the device.

973. The device of item 807, further comprising a coating, wherein the coating completely covers the device.

974. The device of item 807, further comprising a coating, wherein the coating is a uniform coating.

975. The device of item 807, further comprising a coating, wherein the coating is a non-uniform coating.

976. The device of item 807, further comprising a coating, wherein the coating is a discontinuous coating.

977. The device of item 807, further comprising a coating, wherein the coating is a patterned coating.

978. The device of item 807, further comprising a coating, wherein the coating has a thickness of 100 μm or less.

979. The device of item 807, further comprising a coating, wherein the coating has a thickness of 10 μm or less.

980. The device of item 807, further comprising a coating, wherein the coating adheres to the surface of the device upon deployment of the device.

981. The device of item 807, further comprising a coating, wherein the coating is stable at room temperature for a period of 1 year.

982. The device of item 807, further comprising a coating, wherein the anti-scarring agent is present in the coating in an amount ranging between about 0.0001% to about 1% by weight.

983. The device of item 807, further comprising a coating, wherein the anti-scarring agent is present in the coating in an amount ranging between about 1% to about 10% by weight.

984. The device of item 807, further comprising a coating, wherein the anti-scarring agent is present in the coating in an amount ranging between about 10% to about 25% by weight.

985. The device of item 807, further comprising a coating, wherein the anti-scarring agent is present in the coating in an amount ranging between about 25% to about 70% by weight.

986. The device of item 807, further comprising a coating, wherein the coating further comprises a polymer.

987. The device of item 807, further comprising a first coating having a first composition and the second coating having a second composition.

988. The device of item 807, further comprising a first coating having a first composition and the second coating having a second composition, wherein the first composition and the second composition are different.

989. The device of item 807, further comprising a polymer.

990. The device of item 807, further comprising a polymeric carrier.

991. The device of item 807, further comprising a polymeric carrier, wherein the polymeric carrier comprises a copolymer.

992. The device of item 807, further comprising a polymeric carrier, wherein the polymeric carrier comprises a block copolymer.

993. The device of item 807, further comprising a polymeric carrier, wherein the polymeric carrier comprises a random copolymer.

994. The device of item 807, further comprising a polymeric carrier, wherein the polymeric carrier comprises a biodegradable polymer.

995. The device of item 807, further comprising a polymeric carrier, wherein the polymeric carrier comprises a non-biodegradable polymer.

996. The device of item 807, further comprising a polymeric carrier, wherein the polymeric carrier comprises a hydrophilic polymer.

997. The device of item 807, further comprising a polymeric carrier, wherein the polymeric carrier comprises a hydrophobic polymer.

998. The device of item 807, further comprising a polymeric carrier, wherein the polymeric carrier comprises a polymer having hydrophilic domains.

999. The device of item 807, further comprising a polymeric carrier, wherein the polymeric carrier comprises a polymer having hydrophobic domains.

1000. The device of item 807, further comprising a polymeric carrier, wherein the polymeric carrier comprises a non-conductive polymer.

1001. The device of item 807, further comprising a polymeric carrier, wherein the polymeric carrier comprises an elastomer.

1002. The device of item 807, further comprising a polymeric carrier, wherein the polymeric carrier comprises a hydrogel.

1003. The device of item 807, further comprising a polymeric carrier, wherein the polymeric carrier comprises a silicone polymer.

1004. The device of item 807, further comprising a polymeric carrier, wherein the polymeric carrier comprises a hydrocarbon polymer.

1005. The device of item 807, further comprising a polymeric carrier, wherein the polymeric carrier comprises a styrene-derived polymer.

1006. The device of item 807, further comprising a polymeric carrier, wherein the polymeric carrier comprises a butadiene polymer.

1007. The device of item 807, further comprising a polymeric carrier, wherein the polymeric carrier comprises a macromer.

1008. The device of item 807, further comprising a polymeric carrier, wherein the polymeric carrier comprises a poly(ethylene glycol)polymer.

1009. The device of item 807, further comprising a polymeric carrier, wherein the polymeric carrier comprises an amorphous polymer.

1010. The device of item 807, further comprising a lubricious coating.

1011. The device of item 807 wherein the anti-scarring agent is located within pores or holes of the device.

1012. The device of item 807 wherein the anti-scarring agent is located within a channel, lumen, or divet of the device.

1013. The device of item 807, further comprising a second pharmaceutically active agent.

1014. The device of item 807, further comprising an anti-inflammatory agent.

1015. The device of item 807, further comprising an agent that inhibits infection.

1016. The device of item 807, further comprising an agent that inhibits infection, wherein the agent is an anthracycline.

1017. The device of item 807, further comprising an agent that inhibits infection, wherein the agent is doxorubicin.

1018. The device of item 807, further comprising an agent that inhibits infection, wherein the agent is mitoxantrone.

1019. The device of item 807, further comprising an agent that inhibits infection, wherein the agent is a fluoropyrimidine.

1020. The device of item 807, further comprising an agent that inhibits infection, wherein the agent is 5-fluorouracil (5-FU).

1021. The device of item 807, further comprising an agent that inhibits infection, wherein the agent is a folic acid antagonist.

1022. The device of item 807, further comprising an agent that inhibits infection, wherein the agent is methotrexate.

1023. The device of item 807, further comprising an agent that inhibits infection, wherein the agent is a podophylotoxin.

1024. The device of item 807, further comprising an agent that inhibits infection, wherein the agent is etoposide.

1025. The device of item 807, further comprising an agent that inhibits infection, wherein the agent is a camptothecin.

1026. The device of item 807, further comprising an agent that inhibits infection, wherein the agent is a hydroxyurea.

1027. The device of item 807, further comprising an agent that inhibits infection, wherein the agent is a platinum complex.

1028. The device of item 807, further comprising an agent that inhibits infection, wherein the agent is cisplatin.

1029. The device of item 807, further comprising an anti-thrombotic agent.

1030. The device of item 807, further comprising a visualization agent.

1031. The device of item 807, further comprising a visualization agent, wherein the visualization agent is a radiopaque material, wherein the radiopaque material comprises a metal, a halogenated compound, or a barium containing compound.

1032. The device of item 807, further comprising a visualization agent, wherein the visualization agent is a radiopaque material, wherein the radiopaque material comprises barium, tantalum, or technetium.

1033. The device of item 807, further comprising a visualization agent, wherein the visualization agent is a MRI responsive material.

1034. The device of item 807, further comprising a visualization agent, wherein the visualization agent comprises a gadolinium chelate.

1035. The device of item 807, further comprising a visualization agent, wherein the visualization agent comprises iron, magnesium, manganese, copper, or chromium.

1036. The device of item 807, further comprising a visualization agent, wherein the visualization agent comprises an iron oxide compound.

1037. The device of item 807, further comprising a visualization agent, wherein the visualization agent comprises a dye, pigment, or colorant.

1038. The device of item 807, further comprising an echogenic material.

1039. The device of item 807, further comprising an echogenic material, wherein the echogenic material is in the form of a coating.

1040. The device of item 807 wherein the device is sterile.

1041. The device of item 807 wherein the anti-scarring agent is released into tissue in the vicinity of the device after deployment of the device.

1042. The device of item 807 wherein the anti-scarring agent is released into tissue in the vicinity of the device after deployment of the device, wherein the tissue is connective tissue.

1043. The device of item 807 wherein the anti-scarring agent is released into tissue in the vicinity of the device after deployment of the device, wherein the tissue is muscle tissue.

1044. The device of item 807 wherein the anti-scarring agent is released into tissue in the vicinity of the device after deployment of the device, wherein the tissue is nerve tissue.

1045. The device of item 807 wherein the anti-scarring agent is released into tissue in the vicinity of the device after deployment of the device, wherein the tissue is epithelium tissue.

1046. The device of item 807 wherein the anti-scarring agent is released in effective concentrations from the device over a period ranging from the time of deployment of the device to about 1 year.

1047. The device of item 807 wherein the anti-scarring agent is released in effective concentrations from the device over a period ranging from about 1 month to 6 months.

1048. The device of item 807 wherein the anti-scarring agent is released in effective concentrations from the device over a period ranging from about 1-90 days.

1049. The device of item 807 wherein the anti-scarring agent is released in effective concentrations from the device at a constant rate.

1050. The device of item 807 wherein the anti-scarring agent is released in effective concentrations from the device at an increasing rate.

1051. The device of item 807 wherein the anti-scarring agent is released in effective concentrations from the device at a decreasing rate.

1052. The device of item 807 wherein the anti-scarring agent is released in effective concentrations from the composition comprising the anti-scarring agent by diffusion over a period ranging from the time of deployment of the device to about 90 days.

1053. The device of item 807 wherein the anti-scarring agent is released in effective concentrations from the composition comprising the anti-scarring agent by erosion of the composition over a period ranging from the time of deployment of the device to about 90 days.

1054. The device of item 807 wherein the device comprises about 0.01 μg to about 10 μg of the anti-scarring agent.

1055. The device of item 807 wherein the device comprises about 10 μg to about 10 mg of the anti-scarring agent.

1056. The device of item 807 wherein the device comprises about 10 mg to about 250 mg of the anti-scarring agent.

1057. The device of item 807 wherein the device comprises about 250 mg to about 1000 mg of the anti-scarring agent.

1058. The device of item 807 wherein the device comprises about 1000 mg to about 2500 mg of the anti-scarring agent.

1059. The device of item 807 wherein a surface of the device comprises less than 0.01 μg of the anti-scarring agent per mm2 of device surface to which the anti-scarring agent is applied.

1060. The device of item 807 wherein a surface of the device comprises about 0.01 μg to about 1 μg of the anti-scarring agent per mm2 of device surface to which the anti-scarring agent is applied.

1061. The device of item 807 wherein a surface of the device comprises about 1 μg to about 10 μg of the anti-scarring agent per mm2 of device surface to which the anti-scarring agent is applied.

1062. The device of item 807 wherein a surface of the device comprises about 10 μg to about 250 μg of the anti-scarring agent per mm2 of device surface to which the anti-scarring agent is applied.

1063. The device of item 807 wherein a surface of the device comprises about 250 μg to about 1000 μg of the anti-scarring agent of anti-scarring agent per mm2 of device surface to which the anti-scarring agent is applied.

1064. The device of item 807 wherein a surface of the device comprises about 1000 μg to about 2500 μg of the anti-scarring agent per mm2 of device surface to which the anti-scarring agent is applied.

1065. A device, comprising a ventricular assist implant (i.e., a ventricular assist device) and an anti-scarring agent or a composition comprising an anti-scarring agent, wherein the agent inhibits scarring between the device and a host into which the device is implanted.

1066. The device of item 1065 wherein the agent inhibits cell regeneration.

1067. The device of item 1065 wherein the agent inhibits angiogenesis.

1068. The device of item 1065 wherein the agent inhibits fibroblast migration.

1069. The device of item 1065 wherein the agent inhibits fibroblast proliferation.

1070. The device of item 1065 wherein the agent inhibits deposition of extracellular matrix.

1071. The device of item 1065 wherein the agent inhibits tissue remodeling.

1072. The device of item 1065 wherein the agent is an angiogenesis inhibitor.

1073. The device of item 1065 wherein the agent is a 5-lipoxygenase inhibitor or antagonist.

1074. The device of item 1065 wherein the agent is a chemokine receptor antagonist.

1075. The device of item 1065 wherein the agent is a cell cycle inhibitor.

1076. The device of item 1065 wherein the agent is a taxane.

1077. The device of item 1065 wherein the agent is an anti-microtubule agent.

1078. The device of item 1065 wherein the agent is paclitaxel.

1079. The device of item 1065 wherein the agent is not paclitaxel.

1080. The device of item 1065 wherein the agent is an analogue or derivative of paclitaxel.

1081. The device of item 1065 wherein the agent is a vinca alkaloid.

1082. The device of item 1065 wherein the agent is camptothecin or an analogue or derivative thereof.

1083. The device of item 1065 wherein the agent is a podophyllotoxin.

1084. The device of item 1065 wherein the agent is a podophyllotoxin, wherein the podophyllotoxin is etoposide or an analogue or derivative thereof.

1085. The device of item 1065 wherein the agent is an anthracycline.

1086. The device of item 1065 wherein the agent is an anthracycline, wherein the anthracycline is doxorubicin or an analogue or derivative thereof.

1087. The device of item 1065 wherein the agent is an anthracycline, wherein the anthracycline is mitoxantrone or an analogue or derivative thereof.

1088. The device of item 1065 wherein the agent is a platinum compound.

1089. The device of item 1065 wherein the agent is a nitrosourea.

1090. The device of item 1065 wherein the agent is a nitroimidazole.

1091. The device of item 1065 wherein the agent is a folic acid antagonist.

1092. The device of item 1065 wherein the agent is a cytidine analogue.

1093. The device of item 1065 wherein the agent is a pyrimidine analogue.

1094. The device of item 1065 wherein the agent is a fluoropyrimidine analogue.

1095. The device of item 1065 wherein the agent is a purine analogue.

1096. The device of item 1065 wherein the agent is a nitrogen mustard or an analogue or derivative thereof.

1097. The device of item 1065 wherein the agent is a hydroxyurea.

1098. The device of item 1065 wherein the agent is a mytomicin or an analogue or derivative thereof.

1099. The device of item 1065 wherein the agent is an alkyl sulfonate.

1100. The device of item 1065 wherein the agent is a benzamide or an analogue or derivative thereof.

1101. The device of item 1065 wherein the agent is a nicotinamide or an analogue or derivative thereof.

1102. The device of item 1065 wherein the agent is a halogenated sugar or an analogue or derivative thereof.

1103. The device of item 1065 wherein the agent is a DNA alkylating agent.

1104. The device of item 1065 wherein the agent is an anti-microtubule agent.

1105. The device of item 1065 wherein the agent is a topoisomerase inhibitor.

1106. The device of item 1065 wherein the agent is a DNA cleaving agent.

1107. The device of item 1065 wherein the agent is an antimetabolite.

1108. The device of item 1065 wherein the agent inhibits adenosine deaminase.

1109. The device of item 1065 wherein the agent inhibits purine ring synthesis.

1110. The device of item 1065 wherein the agent is a nucleotide interconversion inhibitor.

1111. The device of item 1065 wherein the agent inhibits dihydrofolate reduction.

1112. The device of item 1065 wherein the agent blocks thymidine mono phosphate.

1113. The device of item 1065 wherein the agent causes DNA damage.

1114. The device of item 1065 wherein the agent is a DNA intercalation agent.

1115. The device of item 1065 wherein the agent is a RNA synthesis inhibitor.

1116. The device of item 1065 wherein the agent is a pyrimidine synthesis inhibitor.

1117. The device of item 1065 wherein the agent inhibits ribonucleotide synthesis or function.

1118. The device of item 1065 wherein the agent inhibits thymidine monophosphate synthesis or function.

1119. The device of item 1065 wherein the agent inhibits DNA synthesis.

1120. The device of item 1065 wherein the agent causes DNA adduct formation.

1121. The device of item 1065 wherein the agent inhibits protein synthesis.

1122. The device of item 1065 wherein the agent inhibits microtubule function.

1123. The device of item 1065 wherein the agent is a cyclin dependent protein kinase inhibitor.

1124. The device of item 1065 wherein the agent is an epidermal growth factor kinase inhibitor.

1125. The device of item 1065 wherein the agent is an elastase inhibitor.

1126. The device of item 1065 wherein the agent is a factor Xa inhibitor.

1127. The device of item 1065 wherein the agent is a farnesyltransferase inhibitor.

1128. The device of item 1065 wherein the agent is a fibrinogen antagonist.

1129. The device of item 1065 wherein the agent is a guanylate cyclase stimulant.

1130. The device of item 1065 wherein the agent is a heat shock protein 90 antagonist.

1131. The device of item 1065 wherein the agent is a heat shock protein 90 antagonist, wherein the heat shock protein 90 antagonist is geldanamycin or an analogue or derivative thereof.

1132. The device of item 1065 wherein the agent is a guanylate cyclase stimulant.

1133. The device of item 1065 wherein the agent is a HMGCoA reductase inhibitor.

1134. The device of item 1065 wherein the agent is a HMGCoA reductase inhibitor, wherein the HMGCoA reductase inhibitor is simvastatin or an analogue or derivative thereof.

1135. The device of item 1065 wherein the agent is a hydroorotate dehydrogenase inhibitor.

1136. The device of item 1065 wherein the agent is an IKK2 inhibitor.

1137. The device of item 1065 wherein the agent is an IL-1 antagonist.

1138. The device of item 1065 wherein the agent is an ICE antagonist.

1139. The device of item 1065 wherein the agent is an IRAK antagonist.

1140. The device of item 1065 wherein the agent is an IL-4 agonist.

1141. The device of item 1065 wherein the agent is an immunomodulatory agent.

1142. The device of item 1065 wherein the agent is sirolimus or an analogue or derivative thereof.

1143. The device of item 1065 wherein the agent is not sirolimus.

1144. The device of item 1065 wherein the agent is everolimus or an analogue or derivative thereof.

1145. The device of item 1065 wherein the agent is tacrolimus or an analogue or derivative thereof.

1146. The device of item 1065 wherein the agent is not tacrolimus.

1147. The device of item 1065 wherein the agent is biolmus or an analogue or derivative thereof.

1148. The device of item 1065 wherein the agent is tresperimus or an analogue or derivative thereof.

1149. The device of item 1065 wherein the agent is auranofin or an analogue or derivative thereof.

1150. The device of item 1065 wherein the agent is 27-0-demethylrapamycin or an analogue or derivative thereof.

1151. The device of item 1065 wherein the agent is gusperimus or an analogue or derivative thereof.

1152. The device of item 1065 wherein the agent is pimecrolimus or an analogue or derivative thereof.

1153. The device of item 1065 wherein the agent is ABT-578 or an analogue or derivative thereof.

1154. The device of item 1065 wherein the agent is an inosine monophosphate dehydrogenase (IMPDH) inhibitor.

1155. The device of item 1065 wherein the agent is an IMPDH inhibitor, wherein the IMPDH inhibitor is mycophenolic acid or an analogue or derivative thereof.

1156. The device of item 1065 wherein the agent is an IMPDH inhibitor, wherein the IMPDH inhibitor is 1-alpha-25 dihydroxy vitamin D3 or an analogue or derivative thereof.

1157. The device of item 1065 wherein the agent is a leukotriene inhibitor.

1158. The device of item 1065 wherein the agent is a MCP-1 antagonist.

1159. The device of item 1065 wherein the agent is a MMP inhibitor.

1160. The device of item 1065 wherein the agent is an NF kappa B inhibitor.

1161. The device of item 1065 wherein the agent is an NF kappa B inhibitor, wherein the NF kappa B inhibitor is Bay 11-7082.

1162. The device of item 1065 wherein the agent is an NO agonist.

1163. The device of item 1065 wherein the agent is a p38 MAP kinase inhibitor.

1164. The device of item 1065 wherein the agent is a p38 MAP kinase inhibitor, wherein the p38 MAP kinase inhibitor is SB 202190.

1165. The device of item 1065 wherein the agent is a phosphodiesterase inhibitor.

1166. The device of item 1065 wherein the agent is a TGF beta inhibitor.

1167. The device of item 1065 wherein the agent is a thromboxane A2 antagonist.

1168. The device of item 1065 wherein the agent is a TNFa antagonist.

1169. The device of item 1065 wherein the agent is a TACE inhibitor.

1170. The device of item 1065 wherein the agent is a tyrosine kinase inhibitor.

1171. The device of item 1065 wherein the agent is a vitronectin inhibitor.

1172. The device of item 1065 wherein the agent is a fibroblast growth factor inhibitor.

1173. The device of item 1065 wherein the agent is a protein kinase inhibitor.

1174. The device of item 1065 wherein the agent is a PDGF receptor kinase inhibitor.

1175. The device of item 1065 wherein the agent is an endothelial growth factor receptor kinase inhibitor.

1176. The device of item 1065 wherein the agent is a retinoic acid receptor antagonist.

1177. The device of item 1065 wherein the agent is a platelet derived growth factor receptor kinase inhibitor.

1178. The device of item 1065 wherein the agent is a fibronogin antagonist.

1179. The device of item 1065 wherein the agent is an antimycotic agent.

1180. The device of item 1065 wherein the agent is an antimycotic agent, wherein the antimycotic agent is sulconizole.

1181. The device of item 1065 wherein the agent is a bisphosphonate.

1182. The device of item 1065 wherein the agent is a phospholipase A1 inhibitor.

1183. The device of item 1065 wherein the agent is a histamine H1/H2/H3 receptor antagonist.

1184. The device of item 1065 wherein the agent is a macrolide antibiotic.

1185. The device of item 1065 wherein the agent is a GPIIb/IIIa receptor antagonist.

1186. The device of item 1065 wherein the agent is an endothelin receptor antagonist.

1187. The device of item 1065 wherein the agent is a peroxisome proliferator-activated receptor agonist.

1188. The device of item 1065 wherein the agent is an estrogen receptor agent.

1189. The device of item 1065 wherein the agent is a somastostatin analogue.

1190. The device of item 1065 wherein the agent is a neurokinin 1 antagonist.

1191. The device of item 1065 wherein the agent is a neurokinin 3 antagonist.

1192. The device of item 1065 wherein the agent is a VLA-4 antagonist.

1193. The device of item 1065 wherein the agent is an osteoclast inhibitor.

1194. The device of item 1065 wherein the agent is a DNA topoisomerase ATP hydrolyzing inhibitor.

1195. The device of item 1065 wherein the agent is an angiotensin I converting enzyme inhibitor.

1196. The device of item 1065 wherein the agent is an angiotensin II antagonist.

1197. The device of item 1065 wherein the agent is an enkephalinase inhibitor.

1198. The device of item 1065 wherein the agent is a peroxisome proliferator-activated receptor gamma agonist insulin sensitizer.

1199. The device of item 1065 wherein the agent is a protein kinase C inhibitor.

1200. The device of item 1065 wherein the agent is a ROCK (rho-associated kinase) inhibitor.

1201. The device of item 1065 wherein the agent is a CXCR3 inhibitor.

1202. The device of item 1065 wherein the agent is an Itk inhibitor.

1203. The device of item 1065 wherein the agent is a cytosolic phospholipase A2-alpha inhibitor.

1204. The device of item 1065 wherein the agent is a PPAR agonist.

1205. The device of item 1065 wherein the agent is an immunosuppressant.

1206. The device of item 1065 wherein the agent is an Erb inhibitor.

1207. The device of item 1065 wherein the agent is an apoptosis agonist.

1208. The device of item 1065 wherein the agent is a lipocortin agonist.

1209. The device of item 1065 wherein the agent is a VCAM-1 antagonist.

1210. The device of item 1065 wherein the agent is a collagen antagonist.

1211. The device of item 1065 wherein the agent is an alpha 2 integrin antagonist.

1212. The device of item 1065 wherein the agent is a TNF alpha inhibitor.

1213. The device of item 1065 wherein the agent is a nitric oxide inhibitor.

1214. The device of item 1065 wherein the agent is a cathepsin inhibitor.

1215. The device of item 1065 wherein the agent is not an anti-inflammatory agent.

1216. The device of item 1065 wherein the agent is not a steroid.

1217. The device of item 1065 wherein the agent is not a glucocorticosteroid.

1218. The device of item 1065 wherein the agent is not dexamethasone.

1219. The device of item 1065 wherein the agent is not an anti-infective agent.

1220. The device of item 1065 wherein the agent is not an antibiotic.

1221. The device of item 1065 wherein the agent is not an anti-fungal agent.

1222. The device of item 1065, further comprising a polymer.

1223. The device of item 1065, further comprising a polymeric carrier.

1224. The device of item 1065 wherein the anti-scarring agent inhibits adhesion between the device and a host into which the device is implanted.

1225. The device of item 1065 wherein the device delivers the anti-scarring agent locally to tissue proximate to the device.

1226. The device of item 1065, further comprising a coating, wherein the coating comprises the anti-scarring agent.

1227. The device of item 1065, further comprising a coating, wherein the coating is disposed on a surface of the device.

1228. The device of item 1065, further comprising a coating, wherein the coating directly contacts the device.

1229. The device of item 1065, further comprising a coating, wherein the coating indirectly contacts the device.

1230. The device of item 1065, further comprising a coating, wherein the coating partially covers the device.

1231. The device of item 1065, further comprising a coating, wherein the coating completely covers the device.

1232. The device of item 1065, further comprising a coating, wherein the coating is a uniform coating.

1233. The device of item 1065, further comprising a coating, wherein the coating is a non-uniform coating.

1234. The device of item 1065, further comprising a coating, wherein the coating is a discontinuous coating.

1235. The device of item 1065, further comprising a coating, wherein the coating is a patterned coating.

1236. The device of item 1065, further comprising a coating, wherein the coating has a thickness of 100 μm or less.

1237. The device of item 1065, further comprising a coating, wherein the coating has a thickness of 10 μm or less.

1238. The device of item 1065, further comprising a coating, wherein the coating adheres to the surface of the device upon deployment of the device.

1239. The device of item 1065, further comprising a coating, wherein the coating is stable at room temperature for a period of 1 year.

1240. The device of item 1065, further comprising a coating, wherein the anti-scarring agent is present in the coating in an amount ranging between about 0.0001% to about 1% by weight.

1241. The device of item 1065, further comprising a coating, wherein the anti-scarring agent is present in the coating in an amount ranging between about 1% to about 10% by weight.

1242. The device of item 1065, further comprising a coating, wherein the anti-scarring agent is present in the coating in an amount ranging between about 10% to about 25% by weight.

1243. The device of item 1065, further comprising a coating, wherein the anti-scarring agent is present in the coating in an amount ranging between about 25% to about 70% by weight.

1244. The device of item 1065, further comprising a coating, wherein the coating further comprises a polymer.

1245. The device of item 1065, further comprising a first coating having a first composition and the second coating having a second composition.

1246. The device of item 1065, further comprising a first coating having a first composition and the second coating having a second composition, wherein the first composition and the second composition are different.

1247. The device of item 1065, further comprising a polymer.

1248. The device of item 1065, further comprising a polymeric carrier.

1249. The device of item 1065, further comprising a polymeric carrier, wherein the polymeric carrier comprises a copolymer.

1250. The device of item 1065, further comprising a polymeric carrier, wherein the polymeric carrier comprises a block copolymer.

1251. The device of item 1065, further comprising a polymeric carrier, wherein the polymeric carrier comprises a random copolymer.

1252. The device of item 1065, further comprising a polymeric carrier, wherein the polymeric carrier comprises a biodegradable polymer.

1253. The device of item 1065, further comprising a polymeric carrier, wherein the polymeric carrier comprises a non-biodegradable polymer.

1254. The device of item 1065, further comprising a polymeric carrier, wherein the polymeric carrier comprises a hydrophilic polymer.

1255. The device of item 1065, further comprising a polymeric carrier, wherein the polymeric carrier comprises a hydrophobic polymer.

1256. The device of item 1065, further comprising a polymeric carrier, wherein the polymeric carrier comprises a polymer having hydrophilic domains.

1257. The device of item 1065, further comprising a polymeric carrier, wherein the polymeric carrier comprises a polymer having hydrophobic domains.

1258. The device of item 1065, further comprising a polymeric carrier, wherein the polymeric carrier comprises a non-conductive polymer.

1259. The device of item 1065, further comprising a polymeric carrier, wherein the polymeric carrier comprises an elastomer.

1260. The device of item 1065, further comprising a polymeric carrier, wherein the polymeric carrier comprises a hydrogel.

1261. The device of item 1065, further comprising a polymeric carrier, wherein the polymeric carrier comprises a silicone polymer.

1262. The device of item 1065, further comprising a polymeric carrier, wherein the polymeric carrier comprises a hydrocarbon polymer.

1263. The device of item 1065, further comprising a polymeric carrier, wherein the polymeric carrier comprises a styrene-derived polymer.

1264. The device of item 1065, further comprising a polymeric carrier, wherein the polymeric carrier comprises a butadiene polymer.

1265. The device of item 1065, further comprising a polymeric carrier, wherein the polymeric carrier comprises a macromer.

1266. The device of item 1065, further comprising a polymeric carrier, wherein the polymeric carrier comprises a poly(ethylene glycol)polymer.

1267. The device of item 1065, further comprising a polymeric carrier, wherein the polymeric carrier comprises an amorphous polymer.

1268. The device of item 1065, further comprising a lubricious coating.

1269. The device of item 1065 wherein the anti-scarring agent is located within pores or holes of the device.

1270. The device of item 1065 wherein the anti-scarring agent is located within a channel, lumen, or divet of the device.

1271. The device of item 1065, further comprising a second pharmaceutically active agent.

1272. The device of item 1065, further comprising an anti-inflammatory agent.

1273. The device of item 1065, further comprising an agent that inhibits infection.

1274. The device of item 1065, further comprising an agent that inhibits infection, wherein the agent is an anthracycline.

1275. The device of item 1065, further comprising an agent that inhibits infection, wherein the agent is doxorubicin.

1276. The device of item 1065, further comprising an agent that inhibits infection, wherein the agent is mitoxantrone.

1277. The device of item 1065, further comprising an agent that inhibits infection, wherein the agent is a fluoropyrimidine.

1278. The device of item 1065, further comprising an agent that inhibits infection, wherein the agent is 5-fluorouracil (5-FU).

1279. The device of item 1065, further comprising an agent that inhibits infection, wherein the agent is a folic acid antagonist.

1280. The device of item 1065, further comprising an agent that inhibits infection, wherein the agent is methotrexate.

1281. The device of item 1065, further comprising an agent that inhibits infection, wherein the agent is a podophylotoxin.

1282. The device of item 1065, further comprising an agent that inhibits infection, wherein the agent is etoposide.

1283. The device of item 1065, further comprising an agent that inhibits infection, wherein the agent is a camptothecin.

1284. The device of item 1065, further comprising an agent that inhibits infection, wherein the agent is a hydroxyurea.

1285. The device of item 1065, further comprising an agent that inhibits infection, wherein the agent is a platinum complex.

1286. The device of item 1065, further comprising an agent that inhibits infection, wherein the agent is cisplatin.

1287. The device of item 1065, further comprising an anti-thrombotic agent.

1288. The device of item 1065, further comprising a visualization agent.

1289. The device of item 1065, further comprising a visualization agent, wherein the visualization agent is a radiopaque material, wherein the radiopaque material comprises a metal, a halogenated compound, or a barium containing compound.

1290. The device of item 1065, further comprising a visualization agent, wherein the visualization agent is a radiopaque material, wherein the radiopaque material comprises barium, tantalum, or technetium.

1291. The device of item 1065, further comprising a visualization agent, wherein the visualization agent is a MRI responsive material.

1292. The device of item 1065, further comprising a visualization agent, wherein the visualization agent comprises a gadolinium chelate.

1293. The device of item 1065, further comprising a visualization agent, wherein the visualization agent comprises iron, magnesium, manganese, copper, or chromium.

1294. The device of item 1065, further comprising a visualization agent, wherein the visualization agent comprises an iron oxide compound.

1295. The device of item 1065, further comprising a visualization agent, wherein the visualization agent comprises a dye, pigment, or colorant.

1296. The device of item 1065, further comprising an echogenic material.

1297. The device of item 1065, further comprising an echogenic material, wherein the echogenic material is in the form of a coating.

1298. The device of item 1065 wherein the device is sterile.

1299. The device of item 1065 wherein the anti-scarring agent is released into tissue in the vicinity of the device after deployment of the device.

1300. The device of item 1065 wherein the anti-scarring agent is released into tissue in the vicinity of the device after deployment of the device, wherein the tissue is connective tissue.

1301. The device of item 1065 wherein the anti-scarring agent is released into tissue in the vicinity of the device after deployment of the device, wherein the tissue is muscle tissue.

1302. The device of item 1065 wherein the anti-scarring agent is released into tissue in the vicinity of the device after deployment of the device, wherein the tissue is nerve tissue.

1303. The device of item 1065 wherein the anti-scarring agent is released into tissue in the vicinity of the device after deployment of the device, wherein the tissue is epithelium tissue.

1304. The device of item 1065 wherein the anti-scarring agent is released in effective concentrations from the device over a period ranging from the time of deployment of the device to about 1 year.

1305. The device of item 1065 wherein the anti-scarring agent is released in effective concentrations from the device over a period ranging from about 1 month to 6 months.

1306. The device of item 1065 wherein the anti-scarring agent is released in effective concentrations from the device over a period ranging from about 1-90 days.

1307. The device of item 1065 wherein the anti-scarring agent is released in effective concentrations from the device at a constant rate.

1308. The device of item 1065 wherein the anti-scarring agent is released in effective concentrations from the device at an increasing rate.

1309. The device of item 1065 wherein the anti-scarring agent is released in effective concentrations from the device at a decreasing rate.

1310. The device of item 1065 wherein the anti-scarring agent is released in effective concentrations from the composition comprising the anti-scarring agent by diffusion over a period ranging from the time of deployment of the device to about 90 days.

1311. The device of item 1065 wherein the anti-scarring agent is released in effective concentrations from the composition comprising the anti-scarring agent by erosion of the composition over a period ranging from the time of deployment of the device to about 90 days.

1312. The device of item 1065 wherein the device comprises about 0.01 μg to about 10 μg of the anti-scarring agent.

1313. The device of item 1065 wherein the device comprises about 10 μg to about 10 mg of the anti-scarring agent.

1314. The device of item 1065 wherein the device comprises about 10 mg to about 250 mg of the anti-scarring agent.

1315. The device of item 1065 wherein the device comprises about 250 mg to about 1000 mg of the anti-scarring agent.

1316. The device of item 1065 wherein the device comprises about 1000 mg to about 2500 mg of the anti-scarring agent.

1317. The device of item 1065 wherein a surface of the device comprises less than 0.01 μg of the anti-scarring agent per mm2 of device surface to which the anti-scarring agent is applied.

1318. The device of item 1065 wherein a surface of the device comprises about 0.01 μg to about 1 μg of the anti-scarring agent per mm2 of device surface to which the anti-scarring agent is applied.

1319. The device of item 1065 wherein a surface of the device comprises about 1 μg to about 10 μg of the anti-scarring agent per mm2 of device surface to which the anti-scarring agent is applied.

1320. The device of item 1065 wherein a surface of the device comprises about 10 μl to about 250 μg of the anti-scarring agent per mm2 of device surface to which the anti-scarring agent is applied.

1321. The device of item 1065 wherein a surface of the device comprises about 250 μg to about 1000 μg of the anti-scarring agent of anti-scarring agent per mm2 of device surface to which the anti-scarring agent is applied.

1322. The device of item 1065 wherein a surface of the device comprises about 1000 μg to about 2500 μg of the anti-scarring agent per mm2 of device surface to which the anti-scarring agent is applied.

1323. The device of any one of items 1065-1322 wherein the implant is a left ventricular assist device.

1324. The device of any one of items 1065-1322 wherein the implant is a right ventricular assist device.

1325. The device of any one of items 1065-1322 wherein the implant is a biventricular assist device.

1326. The device of any one of items 1065-1322 wherein the implant is a cardiac assist device.

1327. A device, comprising a prosthetic heart valve implant and an anti-scarring agent or a composition comprising an anti-scarring agent, wherein the agent inhibits scarring between the device and a host into which the device is implanted.

1328. The device of item 1327 wherein the agent inhibits cell regeneration.

1329. The device of item 1327 wherein the agent inhibits angiogenesis.

1330. The device of item 1327 wherein the agent inhibits fibroblast migration.

1331. The device of item 1327 wherein the agent inhibits fibroblast proliferation.

1332. The device of item 1327 wherein the agent inhibits deposition of extracellular matrix.

1333. The device of item 1327 wherein the agent inhibits tissue remodeling.

1334. The device of item 1327 wherein the agent is an angiogenesis inhibitor.

1335. The device of item 1327 wherein the agent is a 5-lipoxygenase inhibitor or antagonist.

1336. The device of item 1327 wherein the agent is a chemokine receptor antagonist.

1337. The device of item 1327 wherein the agent is a cell cycle inhibitor.

1338. The device of item 1327 wherein the agent is a taxane.

1339. The device of item 1327 wherein the agent is an anti-microtubule agent.

1340. The device of item 1327 wherein the agent is paclitaxel.

1341. The device of item 1327 wherein the agent is not paclitaxel.

1342. The device of item 1327 wherein the agent is an analogue or derivative of paclitaxel.

1343. The device of item 1327 wherein the agent is a vinca alkaloid.

1344. The device of item 1327 wherein the agent is camptothecin or an analogue or derivative thereof.

1345. The device of item 1327 wherein the agent is a podophyllotoxin.

1346. The device of item 1327 wherein the agent is a podophyllotoxin, wherein the podophyllotoxin is etoposide or an analogue or derivative thereof.

1347. The device of item 1327 wherein the agent is an anthracycline.

1348. The device of item 1327 wherein the agent is an anthracycline, wherein the anthracycline is doxorubicin or an analogue or derivative thereof.

1349. The device of item 1327 wherein the agent is an anthracycline, wherein the anthracycline is mitoxantrone or an analogue or derivative thereof.

1350. The device of item 1327 wherein the agent is a platinum compound.

1351. The device of item 1327 wherein the agent is a nitrosourea.

1352. The device of item 1327 wherein the agent is a nitroimidazole.

1353. The device of item 1327 wherein the agent is a folic acid antagonist.

1354. The device of item 1327 wherein the agent is a cytidine analogue.

1355. The device of item 1327 wherein the agent is a pyrimidine analogue.

1356. The device of item 1327 wherein the agent is a fluoropyrimidine analogue.

1357. The device of item 1327 wherein the agent is a purine analogue.

1358. The device of item 1327 wherein the agent is a nitrogen mustard or an analogue or derivative thereof.

1359. The device of item 1327 wherein the agent is a hydroxyurea.

1360. The device of item 1327 wherein the agent is a mytomicin or an analogue or derivative thereof.

1361. The device of item 1327 wherein the agent is an alkyl sulfonate.

1362. The device of item 1327 wherein the agent is a benzamide or an analogue or derivative thereof.

1363. The device of item 1327 wherein the agent is a nicotinamide or an analogue or derivative thereof.

1364. The device of item 1327 wherein the agent is a halogenated sugar or an analogue or derivative thereof.

1365. The device of item 1327 wherein the agent is a DNA alkylating agent.

1366. The device of item 1327 wherein the agent is an anti-microtubule agent.

1367. The device of item 1327 wherein the agent is a topoisomerase inhibitor.

1368. The device of item 1327 wherein the agent is a DNA cleaving agent.

1369. The device of item 1327 wherein the agent is an antimetabolite.

1370. The device of item 1327 wherein the agent inhibits adenosine deaminase.

1371. The device of item 1327 wherein the agent inhibits purine ring synthesis.

1372. The device of item 1327 wherein the agent is a nucleotide interconversion inhibitor.

1373. The device of item 1327 wherein the agent inhibits dihydrofolate reduction.

1374. The device of item 1327 wherein the agent blocks thymidine monophosphate.

1375. The device of item 1327 wherein the agent causes DNA damage.

1376. The device of item 1327 wherein the agent is a DNA intercalation agent.

1377. The device of item 1327 wherein the agent is a RNA synthesis inhibitor.

1378. The device of item 1327 wherein the agent is a pyrimidine synthesis inhibitor.

1379. The device of item 1327 wherein the agent inhibits ribonucleotide synthesis or function.

1380. The device of item 1327 wherein the agent inhibits thymidine monophosphate synthesis or function.

1381. The device of item 1327 wherein the agent inhibits DNA synthesis.

1382. The device of item 1327 wherein the agent causes DNA adduct formation.

1383. The device of item 1327 wherein the agent inhibits protein synthesis.

1384. The device of item 1327 wherein the agent inhibits microtubule function.

1385. The device of item 1327 wherein the agent is a cyclin dependent protein kinase inhibitor.

1386. The device of item 1327 wherein the agent is an epidermal growth factor kinase inhibitor.

1387. The device of item 1327 wherein the agent is an elastase inhibitor.

1388. The device of item 1327 wherein the agent is a factor Xa inhibitor.

1389. The device of item 1327 wherein the agent is a farnesyltransferase inhibitor.

1390. The device of item 1327 wherein the agent is a fibrinogen antagonist.

1391. The device of item 1327 wherein the agent is a guanylate cyclase stimulant.

1392. The device of item 1327 wherein the agent is a heat shock protein 90 antagonist.

1393. The device of item 1327 wherein the agent is a heat shock protein 90 antagonist, wherein the heat shock protein 90 antagonist is geldanamycin or an analogue or derivative thereof.

1394. The device of item 1327 wherein the agent is a guanylate cyclase stimulant.

1395. The device of item 1327 wherein the agent is a HMGCoA reductase inhibitor.

1396. The device of item 1327 wherein the agent is a HMGCoA reductase inhibitor, wherein the HMGCoA reductase inhibitor is simvastatin or an analogue or derivative thereof.

1397. The device of item 1327 wherein the agent is a hydroorotate dehydrogenase inhibitor.

1398. The device of item 1327 wherein the agent is an IKK2 inhibitor.

1399. The device of item 1327 wherein the agent is an IL-1 antagonist.

1400. The device of item 1327 wherein the agent is an ICE antagonist.

1401. The device of item 1327 wherein the agent is an IRAK antagonist.

1402. The device of item 1327 wherein the agent is an IL-4 agonist.

1403. The device of item 1327 wherein the agent is an immunomodulatory agent.

1404. The device of item 1327 wherein the agent is sirolimus or an analogue or derivative thereof.

1405. The device of item 1327 wherein the agent is not sirolimus.

1406. The device of item 1327 wherein the agent is everolimus or an analogue or derivative thereof.

1407. The device of item 1327 wherein the agent is tacrolimus or an analogue or derivative thereof.

1408. The device of item 1327 wherein the agent is not tacrolimus.

1409. The device of item 1327 wherein the agent is biolmus or an analogue or derivative thereof.

1410. The device of item 1327 wherein the agent is tresperimus or an analogue or derivative thereof.

1411. The device of item 1327 wherein the agent is auranofin or an analogue or derivative thereof.

1412. The device of item 1327 wherein the agent is 27-0-demethylrapamycin or an analogue or derivative thereof.

1413. The device of item 1327 wherein the agent is gusperimus or an analogue or derivative thereof.

1414. The device of item 1327 wherein the agent is pimecrolimus or an analogue or derivative thereof.

1415. The device of item 1327 wherein the agent is ABT-578 or an analogue or derivative thereof.

1416. The device of item 1327 wherein the agent is an inosine monophosphate dehydrogenase (IMPDH) inhibitor.

1417. The device of item 1327 wherein the agent is an IMPDH inhibitor, wherein the IMPDH inhibitor is mycophenolic acid or an analogue or derivative thereof.

1418. The device of item 1327 wherein the agent is an IMPDH inhibitor, wherein the IMPDH inhibitor is 1-alpha-25 dihydroxy vitamin D3 or an analogue or derivative thereof.

1419. The device of item 1327 wherein the agent is a leukotriene inhibitor.

1420. The device of item 1327 wherein the agent is a MCP-1 antagonist.

1421. The device of item 1327 wherein the agent is a MMP inhibitor.

1422. The device of item 1327 wherein the agent is an NF kappa B inhibitor.

1423. The device of item 1327 wherein the agent is an NF kappa B inhibitor, wherein the NF kappa B inhibitor is Bay 11-7082.

1424. The device of item 1327 wherein the agent is an NO agonist.

1425. The device of item 1327 wherein the agent is a p38 MAP kinase inhibitor.

1426. The device of item 1327 wherein the agent is a p38 MAP kinase inhibitor, wherein the p38 MAP kinase inhibitor is SB 202190.

1427. The device of item 1327 wherein the agent is a phosphodiesterase inhibitor.

1428. The device of item 1327 wherein the agent is a TGF beta inhibitor.

1429. The device of item 1327 wherein the agent is a thromboxane A2 antagonist.

1430. The device of item 1327 wherein the agent is a TNFa antagonist.

1431. The device of item 1327 wherein the agent is a TACE inhibitor.

1432. The device of item 1327 wherein the agent is a tyrosine kinase inhibitor.

1433. The device of item 1327 wherein the agent is a vitronectin inhibitor.

1434. The device of item 1327 wherein the agent is a fibroblast growth factor inhibitor.

1435. The device of item 1327 wherein the agent is a protein kinase inhibitor.

1436. The device of item 1327 wherein the agent is a PDGF receptor kinase inhibitor.

1437. The device of item 1327 wherein the agent is an endothelial growth factor receptor kinase inhibitor.

1438. The device of item 1327 wherein the agent is a retinoic acid receptor antagonist.

1439. The device of item 1327 wherein the agent is a platelet derived growth factor receptor kinase inhibitor.

1440. The device of item 1327 wherein the agent is a fibronogin antagonist.

1441. The device of item 1327 wherein the agent is an antimycotic agent.

1442. The device of item 1327 wherein the agent is an antimycotic agent, wherein the antimycotic agent is sulconizole.

1443. The device of item 1327 wherein the agent is a bisphosphonate.

1444. The device of item 1327 wherein the agent is a phospholipase A1 inhibitor.

1445. The device of item 1327 wherein the agent is a histamine H1/H2/H3 receptor antagonist.

1446. The device of item 1327 wherein the agent is a macrolide antibiotic.

1447. The device of item 1327 wherein the agent is a GPIIb/IIIa receptor antagonist.

1448. The device of item 1327 wherein the agent is an endothelin receptor antagonist.

1449. The device of item 1327 wherein the agent is a peroxisome proliferator-activated receptor agonist.

1450. The device of item 1327 wherein the agent is an estrogen receptor agent.

1451. The device of item 1327 wherein the agent is a somastostatin analogue.

1452. The device of item 1327 wherein the agent is a neurokinin 1 antagonist.

1453. The device of item 1327 wherein the agent is a neurokinin 3 antagonist.

1454. The device of item 1327 wherein the agent is a VLA-4 antagonist.

1455. The device of item 1327 wherein the agent is an osteoclast inhibitor.

1456. The device of item 1327 wherein the agent is a DNA topoisomerase ATP hydrolyzing inhibitor.

1457. The device of item 1327 wherein the agent is an angiotensin I converting enzyme inhibitor.

1458. The device of item 1327 wherein the agent is an angiotensin II antagonist.

1459. The device of item 1327 wherein the agent is an enkephalinase inhibitor.

1460. The device of item 1327 wherein the agent is a peroxisome proliferator-activated receptor gamma agonist insulin sensitizer.

1461. The device of item 1327 wherein the agent is a protein kinase C inhibitor.

1462. The device of item 1327 wherein the agent is a ROCK (rho-associated kinase) inhibitor.

1463. The device of item 1327 wherein the agent is a CXCR3 inhibitor.

1464. The device of item 1327 wherein the agent is an Itk inhibitor.

1465. The device of item 1327 wherein the agent is a cytosolic phospholipase A2-alpha inhibitor.

1466. The device of item 1327 wherein the agent is a PPAR agonist.

1467. The device of item 1327 wherein the agent is an immunosuppressant.

1468. The device of item 1327 wherein the agent is an Erb inhibitor.

1469. The device of item 1327 wherein the agent is an apoptosis agonist.

1470. The device of item 1327 wherein the agent is a lipocortin agonist.

1471. The device of item 1327 wherein the agent is a VCAM-1 antagonist.

1472. The device of item 1327 wherein the agent is a collagen antagonist.

1473. The device of item 1327 wherein the agent is an alpha 2 integrin antagonist.

1474. The device of item 1327 wherein the agent is a TNF alpha inhibitor.

1475. The device of item 1327 wherein the agent is a nitric oxide inhibitor.

1476. The device of item 1327 wherein the agent is a cathepsin inhibitor.

1477. The device of item 1327 wherein the agent is not an anti-inflammatory agent.

1478. The device of item 1327 wherein the agent is not a steroid.

1479. The device of item 1327 wherein the agent is not a glucocorticosteroid.

1480. The device of item 1327 wherein the agent is not dexamethasone.

1481. The device of item 1327 wherein the agent is not an anti-infective agent.

1482. The device of item 1327 wherein the agent is not an antibiotic.

1483. The device of item 1327 wherein the agent is not an anti-fungal agent.

1484. The device of item 1327, further comprising a polymer.

1485. The device of item 1327, further comprising a polymeric carrier.

1486. The device of item 1327 wherein the anti-scarring agent inhibits adhesion between the device and a host into which the device is implanted.

1487. The device of item 1327 wherein the device delivers the anti-scarring agent locally to tissue proximate to the device.

1488. The device of item 1327, further comprising a coating, wherein the coating comprises the anti-scarring agent.

1489. The device of item 1327, further comprising a coating, wherein the coating is disposed on a surface of the device.

1490. The device of item 1327, further comprising a coating, wherein the coating directly contacts the device.

1491. The device of item 1327, further comprising a coating, wherein the coating indirectly contacts the device.

1492. The device of item 1327, further comprising a coating, wherein the coating partially covers the device.

1493. The device of item 1327, further comprising a coating, wherein the coating completely covers the device.

1494. The device of item 1327, further comprising a coating, wherein the coating is a uniform coating.

1495. The device of item 1327, further comprising a coating, wherein the coating is a non-uniform coating.

1496. The device of item 1327, further comprising a coating, wherein the coating is a discontinuous coating.

1497. The device of item 1327, further comprising a coating, wherein the coating is a patterned coating.

1498. The device of item 1327, further comprising a coating, wherein the coating has a thickness of 100 μm or less.

1499. The device of item 1327, further comprising a coating, wherein the coating has a thickness of 10 μm or less.

1500. The device of item 1327, further comprising a coating, wherein the coating adheres to the surface of the device upon deployment of the device.

1501. The device of item 1327, further comprising a coating, wherein the coating is stable at room temperature for a period of 1 year.

1502. The device of item 1327, further comprising a coating, wherein the anti-scarring agent is present in the coating in an amount ranging between about 0.0001% to about 1% by weight.

1503. The device of item 1327, further comprising a coating, wherein the anti-scarring agent is present in the coating in an amount ranging between about 1% to about 10% by weight.

1504. The device of item 1327, further comprising a coating, wherein the anti-scarring agent is present in the coating in an amount ranging between about 10% to about 25% by weight.

1505. The device of item 1327, further comprising a coating, wherein the anti-scarring agent is present in the coating in an amount ranging between about 25% to about 70% by weight.

1506. The device of item 1327, further comprising a coating, wherein the coating further comprises a polymer.

1507. The device of item 1327, further comprising a first coating having a first composition and the second coating having a second composition.

1508. The device of item 1327, further comprising a first coating having a first composition and the second coating having a second composition, wherein the first composition and the second composition are different.

1509. The device of item 1327, further comprising a polymer.

1510. The device of item 1327, further comprising a polymeric carrier.

1511. The device of item 1327, further comprising a polymeric carrier, wherein the polymeric carrier comprises a copolymer.

1512. The device of item 1327, further comprising a polymeric carrier, wherein the polymeric carrier comprises a block copolymer.

1513. The device of item 1327, further comprising a polymeric carrier, wherein the polymeric carrier comprises a random copolymer.

1514. The device of item 1327, further comprising a polymeric carrier, wherein the polymeric carrier comprises a biodegradable polymer.

1515. The device of item 1327, further comprising a polymeric carrier, wherein the polymeric carrier comprises a non-biodegradable polymer.

1516. The device of item 1327, further comprising a polymeric carrier, wherein the polymeric carrier comprises a hydrophilic polymer.

1517. The device of item 1327, further comprising a polymeric carrier, wherein the polymeric carrier comprises a hydrophobic polymer.

1518. The device of item 1327, further comprising a polymeric carrier, wherein the polymeric carrier comprises a polymer having hydrophilic domains.

1519. The device of item 1327, further comprising a polymeric carrier, wherein the polymeric carrier comprises a polymer having hydrophobic domains.

1520. The device of item 1327, further comprising a polymeric carrier, wherein the polymeric carrier comprises a non-conductive polymer.

1521. The device of item 1327, further comprising a polymeric carrier, wherein the polymeric carrier comprises an elastomer.

1522. The device of item 1327, further comprising a polymeric carrier, wherein the polymeric carrier comprises a hydrogel.

1523. The device of item 1327, further comprising a polymeric carrier, wherein the polymeric carrier comprises a silicone polymer.

1524. The device of item 1327, further comprising a polymeric carrier, wherein the polymeric carrier comprises a hydrocarbon polymer.

1525. The device of item 1327, further comprising a polymeric carrier, wherein the polymeric carrier comprises a styrene-derived polymer.

1526. The device of item 1327, further comprising a polymeric carrier, wherein the polymeric carrier comprises a butadiene polymer.

1527. The device of item 1327, further comprising a polymeric carrier, wherein the polymeric carrier comprises a macromer.

1528. The device of item 1327, further comprising a polymeric carrier, wherein the polymeric carrier comprises a poly(ethylene glycol)polymer.

1529. The device of item 1327, further comprising a polymeric carrier, wherein the polymeric carrier comprises an amorphous polymer.

1530. The device of item 1327, further comprising a lubricious coating.

1531. The device of item 1327 wherein the anti-scarring agent is located within pores or holes of the device.

1532. The device of item 1327 wherein the anti-scarring agent is located within a channel, lumen, or divet of the device.

1533. The device of item 1327, further comprising a second pharmaceutically active agent.

1534. The device of item 1327, further comprising an anti-inflammatory agent.

1535. The device of item 1327, further comprising an agent that inhibits infection.

1536. The device of item 1327, further comprising an agent that inhibits infection, wherein the agent is an anthracycline.

1537. The device of item 1327, further comprising an agent that inhibits infection, wherein the agent is doxorubicin.

1538. The device of item 1327, further comprising an agent that inhibits infection, wherein the agent is mitoxantrone.

1539. The device of item 1327, further comprising an agent that inhibits infection, wherein the agent is a fluoropyrimidine.

1540. The device of item 1327, further comprising an agent that inhibits infection, wherein the agent is 5-fluorouracil (5-FU).

1541. The device of item 1327, further comprising an agent that inhibits infection, wherein the agent is a folic acid antagonist.

1542. The device of item 1327, further comprising an agent that inhibits infection, wherein the agent is methotrexate.

1543. The device of item 1327, further comprising an agent that inhibits infection, wherein the agent is a podophylotoxin.

1544. The device of item 1327, further comprising an agent that inhibits infection, wherein the agent is etoposide.

1545. The device of item 1327, further comprising an agent that inhibits infection, wherein the agent is a camptothecin.

1546. The device of item 1327, further comprising an agent that inhibits infection, wherein the agent is a hydroxyurea.

1547. The device of item 1327, further comprising an agent that inhibits infection, wherein the agent is a platinum complex.

1548. The device of item 1327, further comprising an agent that inhibits infection, wherein the agent is cisplatin.

1549. The device of item 1327, further comprising an anti-thrombotic agent.

1550. The device of item 1327, further comprising a visualization agent.

1551. The device of item 1327, further comprising a visualization agent, wherein the visualization agent is a radiopaque material, wherein the radiopaque material comprises a metal, a halogenated compound, or a barium containing compound.

1552. The device of item 1327, further comprising a visualization agent, wherein the visualization agent is a radiopaque material, wherein the radiopaque material comprises barium, tantalum, or technetium.

1553. The device of item 1327, further comprising a visualization agent, wherein the visualization agent is a MRI responsive material.

1554. The device of item 1327, further comprising a visualization agent, wherein the visualization agent comprises a gadolinium chelate.

1555. The device of item 1327, further comprising a visualization agent, wherein the visualization agent comprises iron, magnesium, manganese, copper, or chromium.

1556. The device of item 1327, further comprising a visualization agent, wherein the visualization agent comprises an iron oxide compound.

1557. The device of item 1327, further comprising a visualization agent, wherein the visualization agent comprises a dye, pigment, or colorant.

1558. The device of item 1327, further comprising an echogenic material.

1559. The device of item 1327, further comprising an echogenic material, wherein the echogenic material is in the form of a coating.

1560. The device of item 1327 wherein the device is sterile.

1561. The device of item 1327 wherein the anti-scarring agent is released into tissue in the vicinity of the device after deployment of the device.

1562. The device of item 1327 wherein the anti-scarring agent is released into tissue in the vicinity of the device after deployment of the device, wherein the tissue is connective tissue.

1563. The device of item 1327 wherein the anti-scarring agent is released into tissue in the vicinity of the device after deployment of the device, wherein the tissue is muscle tissue.

1564. The device of item 1327 wherein the anti-scarring agent is released into tissue in the vicinity of the device after deployment of the device, wherein the tissue is nerve tissue.

1565. The device of item 1327 wherein the anti-scarring agent is released into tissue in the vicinity of the device after deployment of the device, wherein the tissue is epithelium tissue.

1566. The device of item 1327 wherein the anti-scarring agent is released in effective concentrations from the device over a period ranging from the time of deployment of the device to about 1 year.

1567. The device of item 1327 wherein the anti-scarring agent is released in effective concentrations from the device over a period ranging from about 1 month to 6 months.

1568. The device of item 1327 wherein the anti-scarring agent is released in effective concentrations from the device over a period ranging from about 1-90 days.

1569. The device of item 1327 wherein the anti-scarring agent is released in effective concentrations from the device at a constant rate.

1570. The device of item 1327 wherein the anti-scarring agent is released in effective concentrations from the device at an increasing rate.

1571. The device of item 1327 wherein the anti-scarring agent is released in effective concentrations from the device at a decreasing rate.

1572. The device of item 1327 wherein the anti-scarring agent is released in effective concentrations from the composition comprising the anti-scarring agent by diffusion over a period ranging from the time of deployment of the device to about 90 days.

1573. The device of item 1327 wherein the anti-scarring agent is released in effective concentrations from the composition comprising the anti-scarring agent by erosion of the composition over a period ranging from the time of deployment of the device to about 90 days.

1574. The device of item 1327 wherein the device comprises about 0.01 μg to about 10 μg of the anti-scarring agent.

1575. The device of item 1327 wherein the device comprises about 10 μg to about 10 mg of the anti-scarring agent.

1576. The device of item 1327 wherein the device comprises about 10 mg to about 250 mg of the anti-scarring agent.

1577. The device of item 1327 wherein the device comprises about 250 mg to about 1000 mg of the anti-scarring agent.

1578. The device of item 1327 wherein the device comprises about 1000 mg to about 2500 mg of the anti-scarring agent.

1579. The device of item 1327 wherein a surface of the device comprises less than 0.01 μg of the anti-scarring agent per mm2 of device surface to which the anti-scarring agent is applied.

1580. The device of item 1327 wherein a surface of the device comprises about 0.01 μg to about 1 μg of the anti-scarring agent per mm2 of device surface to which the anti-scarring agent is applied.

1581. The device of item 1327 wherein a surface of the device comprises about 1 μg to about 10 μg of the anti-scarring agent per mm2 of device surface to which the anti-scarring agent is applied.

1582. The device of item 1327 wherein a surface of the device comprises about 10 μg to about 250 μg of the anti-scarring agent per mm2 of device surface to which the anti-scarring agent is applied.

1583. The device of item 1327 wherein a surface of the device comprises about 250 μg to about 1000 μg of the anti-scarring agent of anti-scarring agent per mm2 of device surface to which the anti-scarring agent is applied.

1584. The device of item 1327 wherein a surface of the device comprises about 1000 μg to about 2500 μg of the anti-scarring agent per mm2 of device surface to which the anti-scarring agent is applied.

1585. The device of any one of items 1327-1584 wherein the implant is a mechanical prosthesis.

1586. The device of any one of items 1327-1584 wherein the implant is a bioprosthetic heart valve.

1587. The device of any one of items 1327-1584 wherein the implant is a bioprosthetic heart valve formed, at least in part, from porcine valve.

1588. The device of any one of items 1327-1584 wherein the implant is a bioprosthetic heart valve formed, at least in part, from bovine pericardial valve.

1589. A device, comprising an inferior vena cava filter implant an anti-scarring agent or a composition comprising an anti-scarring agent, wherein the agent inhibits scarring between the device and a host into which the device is implanted.

1590. The device of item 1589 wherein the agent inhibits cell regeneration.

1591. The device of item 1589 wherein the agent inhibits angiogenesis.

1592. The device of item 1589 wherein the agent inhibits fibroblast migration.

1593. The device of item 1589 wherein the agent inhibits fibroblast proliferation.

1594. The device of item 1589 wherein the agent inhibits deposition of extracellular matrix.

1595. The device of item 1589 wherein the agent inhibits tissue remodeling.

1596. The device of item 1589 wherein the agent is an angiogenesis inhibitor.

1597. The device of item 1589 wherein the agent is a 5-lipoxygenase inhibitor or antagonist.

1598. The device of item 1589 wherein the agent is a chemokine receptor antagonist.

1599. The device of item 1589 wherein the agent is a cell cycle inhibitor.

1600. The device of item 1589 wherein the agent is a taxane.

1601. The device of item 1589 wherein the agent is an anti-microtubule agent.

1602. The device of item 1589 wherein the agent is paclitaxel.

1603. The device of item 1589 wherein the agent is not paclitaxel.

1604. The device of item 1589 wherein the agent is an analogue or derivative of paclitaxel.

1605. The device of item 1589 wherein the agent is a vinca alkaloid.

1606. The device of item 1589 wherein the agent is camptothecin or an analogue or derivative thereof.

1607. The device of item 1589 wherein the agent is a podophyllotoxin.

1608. The device of item 1589 wherein the agent is a podophyllotoxin, wherein the podophyllotoxin is etoposide or an analogue or derivative thereof.

1609. The device of item 1589 wherein the agent is an anthracycline.

1610. The device of item 1589 wherein the agent is an anthracycline, wherein the anthracycline is doxorubicin or an analogue or derivative thereof.

1611. The device of item 1589 wherein the agent is an anthracycline, wherein the anthracycline is mitoxantrone or an analogue or derivative thereof.

1612. The device of item 1589 wherein the agent is a platinum compound.

1613. The device of item 1589 wherein the agent is a nitrosourea.

1614. The device of item 1589 wherein the agent is a nitroimidazole.

1615. The device of item 1589 wherein the agent is a folic acid antagonist.

1616. The device of item 1589 wherein the agent is a cytidine analogue.

1617. The device of item 1589 wherein the agent is a pyrimidine analogue.

1618. The device of item 1589 wherein the agent is a fluoropyrimidine analogue.

1619. The device of item 1589 wherein the agent is a purine analogue.

1620. The device of item 1589 wherein the agent is a nitrogen mustard or an analogue or derivative thereof.

1621. The device of item 1589 wherein the agent is a hydroxyurea.

1622. The device of item 1589 wherein the agent is a mytomicin or an analogue or derivative thereof.

1623. The device of item 1589 wherein the agent is an alkyl sulfonate.

1624. The device of item 1589 wherein the agent is a benzamide or an analogue or derivative thereof.

1625. The device of item 1589 wherein the agent is a nicotinamide or an analogue or derivative thereof.

1626. The device of item 1589 wherein the agent is a halogenated sugar or an analogue or derivative thereof.

1627. The device of item 1589 wherein the agent is a DNA alkylating agent.

1628. The device of item 1589 wherein the agent is an anti-microtubule agent.

1629. The device of item 1589 wherein the agent is a topoisomerase inhibitor.

1630. The device of item 1589 wherein the agent is a DNA cleaving agent.

1631. The device of item 1589 wherein the agent is an antimetabolite.

1632. The device of item 1589 wherein the agent inhibits adenosine deaminase.

1633. The device of item 1589 wherein the agent inhibits purine ring synthesis.

1634. The device of item 1589 wherein the agent is a nucleotide interconversion inhibitor.

1635. The device of item 1589 wherein the agent inhibits dihydrofolate reduction.

1636. The device of item 1589 wherein the agent blocks thymidine mono phosphate.

1637. The device of item 1589 wherein the agent causes DNA damage.

1638. The device of item 1589 wherein the agent is a DNA intercalation agent.

1639. The device of item 1589 wherein the agent is a RNA synthesis inhibitor.

1640. The device of item 1589 wherein the agent is a pyrimidine synthesis inhibitor.

1641. The device of item 1589 wherein the agent inhibits ribonucleotide synthesis or function.

1642. The device of item 1589 wherein the agent inhibits thymidine monophosphate synthesis or function.

1643. The device of item 1589 wherein the agent inhibits DNA synthesis.

1644. The device of item 1589 wherein the agent causes DNA adduct formation.

1645. The device of item 1589 wherein the agent inhibits protein synthesis.

1646. The device of item 1589 wherein the agent inhibits microtubule function.

1647. The device of item 1589 wherein the agent is a cyclin dependent protein kinase inhibitor.

1648. The device of item 1589 wherein the agent is an epidermal growth factor kinase inhibitor.

1649. The device of item 1589 wherein the agent is an elastase inhibitor.

1650. The device of item 1589 wherein the agent is a factor Xa inhibitor.

1651. The device of item 1589 wherein the agent is a farnesyltransferase inhibitor.

1652. The device of item 1589 wherein the agent is a fibrinogen antagonist.

1653. The device of item 1589 wherein the agent is a guanylate cyclase stimulant.

1654. The device of item 1589 wherein the agent is a heat shock protein 90 antagonist.

1655. The device of item 1589 wherein the agent is a heat shock protein 90 antagonist, wherein the heat shock protein 90 antagonist is geldanamycin or an analogue or derivative thereof.

1656. The device of item 1589 wherein the agent is a guanylate cyclase stimulant.

1657. The device of item 1589 wherein the agent is a HMGCoA reductase inhibitor.

1658. The device of item 1589 wherein the agent is a HMGCoA reductase inhibitor, wherein the HMGCoA reductase inhibitor is simvastatin or an analogue or derivative thereof.

1659. The device of item 1589 wherein the agent is a hydroorotate dehydrogenase inhibitor.

1660. The device of item 1589 wherein the agent is an IKK2 inhibitor.

1661. The device of item 1589 wherein the agent is an IL-1 antagonist.

1662. The device of item 1589 wherein the agent is an ICE antagonist.

1663. The device of item 1589 wherein the agent is an IRAK antagonist.

1664. The device of item 1589 wherein the agent is an IL-4 agonist.

1665. The device of item 1589 wherein the agent is an immunomodulatory agent.

1666. The device of item 1589 wherein the agent is sirolimus or an analogue or derivative thereof.

1667. The device of item 1589 wherein the agent is not sirolimus.

1668. The device of item 1589 wherein the agent is everolimus or an analogue or derivative thereof.

1669. The device of item 1589 wherein the agent is tacrolimus or an analogue or derivative thereof.

1670. The device of item 1589 wherein the agent is not tacrolimus.

1671. The device of item 1589 wherein the agent is biolmus or an analogue or derivative thereof.

1672. The device of item 1589 wherein the agent is tresperimus or an analogue or derivative thereof.

1673. The device of item 1589 wherein the agent is auranofin or an analogue or derivative thereof.

1674. The device of item 1589 wherein the agent is 27-0-demethylrapamycin or an analogue or derivative thereof.

1675. The device of item 1589 wherein the agent is gusperimus or an analogue or derivative thereof.

1676. The device of item 1589 wherein the agent is pimecrolimus or an analogue or derivative thereof.

1677. The device of item 1589 wherein the agent is ABT-578 or an analogue or derivative thereof.

1678. The device of item 1589 wherein the agent is an inosine monophosphate dehydrogenase (IMPDH) inhibitor.

1679. The device of item 1589 wherein the agent is an IMPDH inhibitor, wherein the IMPDH inhibitor is mycophenolic acid or an analogue or derivative thereof.

1680. The device of item 1589 wherein the agent is an IMPDH inhibitor, wherein the IMPDH inhibitor is 1-alpha-25 dihydroxy vitamin D3 or an analogue or derivative thereof.

1681. The device of item 1589 wherein the agent is a leukotriene inhibitor.

1682. The device of item 1589 wherein the agent is a MCP-1 antagonist.

1683. The device of item 1589 wherein the agent is a MMP inhibitor.

1684. The device of item 1589 wherein the agent is an NF kappa B inhibitor.

1685. The device of item 1589 wherein the agent is an NF kappa B inhibitor, wherein the NF kappa B inhibitor is Bay 11-7082.

1686. The device of item 1589 wherein the agent is an NO agonist.

1687. The device of item 1589 wherein the agent is a p38 MAP kinase inhibitor.

1688. The device of item 1589 wherein the agent is a p38 MAP kinase inhibitor, wherein the p38 MAP kinase inhibitor is SB 202190.

1689. The device of item 1589 wherein the agent is a phosphodiesterase inhibitor.

1690. The device of item 1589 wherein the agent is a TGF beta inhibitor.

1691. The device of item 1589 wherein the agent is a thromboxane A2 antagonist.

1692. The device of item 1589 wherein the agent is a TNFa antagonist.

1693. The device of item 1589 wherein the agent is a TACE inhibitor.

1694. The device of item 1589 wherein the agent is a tyrosine kinase inhibitor.

1695. The device of item 1589 wherein the agent is a vitronectin inhibitor.

1696. The device of item 1589 wherein the agent is a fibroblast growth factor inhibitor.

1697. The device of item 1589 wherein the agent is a protein kinase inhibitor.

1698. The device of item 1589 wherein the agent is a PDGF receptor kinase inhibitor.

1699. The device of item 1589 wherein the agent is an endothelial growth factor receptor kinase inhibitor.

1700. The device of item 1589 wherein the agent is a retinoic acid receptor antagonist.

1701. The device of item 1589 wherein the agent is a platelet derived growth factor receptor kinase inhibitor.

1702. The device of item 1589 wherein the agent is a fibronogin antagonist.

1703. The device of item 1589 wherein the agent is an antimycotic agent.

1704. The device of item 1589 wherein the agent is an antimycotic agent, wherein the antimycotic agent is sulconizole.

1705. The device of item 1589 wherein the agent is a bisphosphonate.

1706. The device of item 1589 wherein the agent is a phospholipase A1 inhibitor.

1707. The device of item 1589 wherein the agent is a histamine H1/H2/H3 receptor antagonist.

1708. The device of item 1589 wherein the agent is a macrolide antibiotic.

1709. The device of item 1589 wherein the agent is a GPIIb/IIIa receptor antagonist.

1710. The device of item 1589 wherein the agent is an endothelin receptor antagonist.

1711. The device of item 1589 wherein the agent is a peroxisome proliferator-activated receptor agonist.

1712. The device of item 1589 wherein the agent is an estrogen receptor agent.

1713. The device of item 1589 wherein the agent is a somastostatin analogue.

1714. The device of item 1589 wherein the agent is a neurokinin 1 antagonist.

1715. The device of item 1589 wherein the agent is a neurokinin 3 antagonist.

1716. The device of item 1589 wherein the agent is a VLA-4 antagonist.

1717. The device of item 1589 wherein the agent is an osteoclast inhibitor.

1718. The device of item 1589 wherein the agent is a DNA topoisomerase ATP hydrolyzing inhibitor.

1719. The device of item 1589 wherein the agent is an angiotensin I converting enzyme inhibitor.

1720. The device of item 1589 wherein the agent is an angiotensin II antagonist.

1721. The device of item 1589 wherein the agent is an enkephalinase inhibitor.

1722. The device of item 1589 wherein the agent is a peroxisome proliferator-activated receptor gamma agonist insulin sensitizer.

1723. The device of item 1589 wherein the agent is a protein kinase C inhibitor.

1724. The device of item 1589 wherein the agent is a ROCK (rho-associated kinase) inhibitor.

1725. The device of item 1589 wherein the agent is a CXCR3 inhibitor.

1726. The device of item 1589 wherein the agent is an Itk inhibitor.

1727. The device of item 1589 wherein the agent is a cytosolic phospholipase A2-alpha inhibitor.

1728. The device of item 1589 wherein the agent is a PPAR agonist.

1729. The device of item 1589 wherein the agent is an immunosuppressant.

1730. The device of item 1589 wherein the agent is an Erb inhibitor.

1731. The device of item 1589 wherein the agent is an apoptosis agonist.

1732. The device of item 1589 wherein the agent is a lipocortin agonist.

1733. The device of item 1589 wherein the agent is a VCAM-1 antagonist.

1734. The device of item 1589 wherein the agent is a collagen antagonist.

1735. The device of item 1589 wherein the agent is an alpha 2 integrin antagonist.

1736. The device of item 1589 wherein the agent is a TNF alpha inhibitor.

1737. The device of item 1589 wherein the agent is a nitric oxide inhibitor.

1738. The device of item 1589 wherein the agent is a cathepsin inhibitor.

1739. The device of item 1589 wherein the agent is not an anti-inflammatory agent.

1740. The device of item 1589 wherein the agent is not a steroid.

1741. The device of item 1589 wherein the agent is not a glucocorticosteroid.

1742. The device of item 1589 wherein the agent is not dexamethasone.

1743. The device of item 1589 wherein the agent is not an anti-infective agent.

1744. The device of item 1589 wherein the agent is not an antibiotic.

1745. The device of item 1589 wherein the agent is not an anti-fungal agent.

1746. The device of item 1589, further comprising a polymer.

1747. The device of item 1589, further comprising a polymeric carrier.

1748. The device of item 1589 wherein the anti-scarring agent inhibits adhesion between the device and a host into which the device is implanted.

1749. The device of item 1589 wherein the device delivers the anti-scarring agent locally to tissue proximate to the device.

1750. The device of item 1589, further comprising a coating, wherein the coating comprises the anti-scarring agent.

1751. The device of item 1589, further comprising a coating, wherein the coating is disposed on a surface of the device.

1752. The device of item 1589, further comprising a coating, wherein the coating directly contacts the device.

1753. The device of item 1589, further comprising a coating, wherein the coating indirectly contacts the device.

1754. The device of item 1589, further comprising a coating, wherein the coating partially covers the device.

1755. The device of item 1589, further comprising a coating, wherein the coating completely covers the device.

1756. The device of item 1589, further comprising a coating, wherein the coating is a uniform coating.

1757. The device of item 1589, further comprising a coating, wherein the coating is a non-uniform coating.

1758. The device of item 1589, further comprising a coating, wherein the coating is a discontinuous coating.

1759. The device of item 1589, further comprising a coating, wherein the coating is a patterned coating.

1760. The device of item 1589, further comprising a coating, wherein the coating has a thickness of 100 μm or less.

1761. The device of item 1589, further comprising a coating, wherein the coating has a thickness of 10 μm or less.

1762. The device of item 1589, further comprising a coating, wherein the coating adheres to the surface of the device upon deployment of the device.

1763. The device of item 1589, further comprising a coating, wherein the coating is stable at room temperature for a period of 1 year.

1764. The device of item 1589, further comprising a coating, wherein the anti-scarring agent is present in the coating in an amount ranging between about 0.0001% to about 1% by weight.

1765. The device of item 1589, further comprising a coating, wherein the anti-scarring agent is present in the coating in an amount ranging between about 1% to about 10% by weight.

1766. The device of item 1589, further comprising a coating, wherein the anti-scarring agent is present in the coating in an amount ranging between about 10% to about 25% by weight.

1767. The device of item 1589, further comprising a coating, wherein the anti-scarring agent is present in the coating in an amount ranging between about 25% to about 70% by weight.

1768. The device of item 1589, further comprising a coating, wherein the coating further comprises a polymer.

1769. The device of item 1589, further comprising a first coating having a first composition and the second coating having a second composition.

1770. The device of item 1589, further comprising a first coating having a first composition and the second coating having a second composition, wherein the first composition and the second composition are different.

1771. The device of item 1589, further comprising a polymer.

1772. The device of item 1589, further comprising a polymeric carrier.

1773. The device of item 1589, further comprising a polymeric carrier, wherein the polymeric carrier comprises a copolymer.

1774. The device of item 1589, further comprising a polymeric carrier, wherein the polymeric carrier comprises a block copolymer.

1775. The device of item 1589, further comprising a polymeric carrier, wherein the polymeric carrier comprises a random copolymer.

1776. The device of item 1589, further comprising a polymeric carrier, wherein the polymeric carrier comprises a biodegradable polymer.

1777. The device of item 1589, further comprising a polymeric carrier, wherein the polymeric carrier comprises a non-biodegradable polymer.

1778. The device of item 1589, further comprising a polymeric carrier, wherein the polymeric carrier comprises a hydrophilic polymer.

1779. The device of item 1589, further comprising a polymeric carrier, wherein the polymeric carrier comprises a hydrophobic polymer.

1780. The device of item 1589, further comprising a polymeric carrier, wherein the polymeric carrier comprises a polymer having hydrophilic domains.

1781. The device of item 1589, further comprising a polymeric carrier, wherein the polymeric carrier comprises a polymer having hydrophobic domains.

1782. The device of item 1589, further comprising a polymeric carrier, wherein the polymeric carrier comprises a non-conductive polymer.

1783. The device of item 1589, further comprising a polymeric carrier, wherein the polymeric carrier comprises an elastomer.

1784. The device of item 1589, further comprising a polymeric carrier, wherein the polymeric carrier comprises a hydrogel.

1785. The device of item 1589, further comprising a polymeric carrier, wherein the polymeric carrier comprises a silicone polymer.

1786. The device of item 1589, further comprising a polymeric carrier, wherein the polymeric carrier comprises a hydrocarbon polymer.

1787. The device of item 1589, further comprising a polymeric carrier, wherein the polymeric carrier comprises a styrene-derived polymer.

1788. The device of item 1589, further comprising a polymeric carrier, wherein the polymeric carrier comprises a butadiene polymer.

1789. The device of item 1589, further comprising a polymeric carrier, wherein the polymeric carrier comprises a macromer.

1790. The device of item 1589, further comprising a polymeric carrier, wherein the polymeric carrier comprises a poly(ethylene glycol)polymer.

1791. The device of item 1589, further comprising a polymeric carrier, wherein the polymeric carrier comprises an amorphous polymer.

1792. The device of item 1589, further comprising a lubricious coating.

1793. The device of item 1589 wherein the anti-scarring agent is located within pores or holes of the device.

1794. The device of item 1589 wherein the anti-scarring agent is located within a channel, lumen, or divet of the device.

1795. The device of item 1589, further comprising a second pharmaceutically active agent.

1796. The device of item 1589, further comprising an anti-inflammatory agent.

1797. The device of item 1589, further comprising an agent that inhibits infection.

1798. The device of item 1589, further comprising an agent that inhibits infection, wherein the agent is an anthracycline.

1799. The device of item 1589, further comprising an agent that inhibits infection, wherein the agent is doxorubicin.

1800. The device of item 1589, further comprising an agent that inhibits infection, wherein the agent is mitoxantrone.

1801. The device of item 1589, further comprising an agent that inhibits infection, wherein the agent is a fluoropyrimidine.

1802. The device of item 1589, further comprising an agent that inhibits infection, wherein the agent is 5-fluorouracil (5-FU).

1803. The device of item 1589, further comprising an agent that inhibits infection, wherein the agent is a folic acid antagonist.

1804. The device of item 1589, further comprising an agent that inhibits infection, wherein the agent is methotrexate.

1805. The device of item 1589, further comprising an agent that inhibits infection, wherein the agent is a podophylotoxin.

1806. The device of item 1589, further comprising an agent that inhibits infection, wherein the agent is etoposide.

1807. The device of item 1589, further comprising an agent that inhibits infection, wherein the agent is a camptothecin.

1808. The device of item 1589, further comprising an agent that inhibits infection, wherein the agent is a hydroxyurea.

1809. The device of item 1589, further comprising an agent that inhibits infection, wherein the agent is a platinum complex.

1810. The device of item 1589, further comprising an agent that inhibits infection, wherein the agent is cisplatin.

1811. The device of item 1589, further comprising an anti-thrombotic agent.

1812. The device of item 1589, further comprising a visualization agent.

1813. The device of item 1589, further comprising a visualization agent, wherein the visualization agent is a radiopaque material, wherein the radiopaque material comprises a metal, a halogenated compound, or a barium containing compound.

1814. The device of item 1589, further comprising a visualization agent, wherein the visualization agent is a radiopaque material, wherein the radiopaque material comprises barium, tantalum, or technetium.

1815. The device of item 1589, further comprising a visualization agent, wherein the visualization agent is a MRI responsive material.

1816. The device of item 1589, further comprising a visualization agent, wherein the visualization agent comprises a gadolinium chelate.

1817. The device of item 1589, further comprising a visualization agent, wherein the visualization agent comprises iron, magnesium, manganese, copper, or chromium.

1818. The device of item 1589, further comprising a visualization agent, wherein the visualization agent comprises an iron oxide compound.

1819. The device of item 1589, further comprising a visualization agent, wherein the visualization agent comprises a dye, pigment, or colorant.

1820. The device of item 1589, further comprising an echogenic material.

1821. The device of item 1589, further comprising an echogenic material, wherein the echogenic material is in the form of a coating.

1822. The device of item 1589 wherein the device is sterile.

1823. The device of item 1589 wherein the anti-scarring agent is released into tissue in the vicinity of the device after deployment of the device.

1824. The device of item 1589 wherein the anti-scarring agent is released into tissue in the vicinity of the device after deployment of the device, wherein the tissue is connective tissue.

1825. The device of item 1589 wherein the anti-scarring agent is released into tissue in the vicinity of the device after deployment of the device, wherein the tissue is muscle tissue.

1826. The device of item 1589 wherein the anti-scarring agent is released into tissue in the vicinity of the device after deployment of the device, wherein the tissue is nerve tissue.

1827. The device of item 1589 wherein the anti-scarring agent is released into tissue in the vicinity of the device after deployment of the device, wherein the tissue is epithelium tissue.

1828. The device of item 1589 wherein the anti-scarring agent is released in effective concentrations from the device over a period ranging from the time of deployment of the device to about 1 year.

1829. The device of item 1589 wherein the anti-scarring agent is released in effective concentrations from the device over a period ranging from about 1 month to 6 months.

1830. The device of item 1589 wherein the anti-scarring agent is released in effective concentrations from the device over a period ranging from about 1-90 days.

1831. The device of item 1589 wherein the anti-scarring agent is released in effective concentrations from the device at a constant rate.

1832. The device of item 1589 wherein the anti-scarring agent is released in effective concentrations from the device at an increasing rate.

1833. The device of item 1589 wherein the anti-scarring agent is released in effective concentrations from the device at a decreasing rate.

1834. The device of item 1589 wherein the anti-scarring agent is released in effective concentrations from the composition comprising the anti-scarring agent by diffusion over a period ranging from the time of deployment of the device to about 90 days.

1835. The device of item 1589 wherein the anti-scarring agent is released in effective concentrations from the composition comprising the anti-scarring agent by erosion of the composition over a period ranging from the time of deployment of the device to about 90 days.

1836. The device of item 1589 wherein the device comprises about 0.01 μg to about 10 μg of the anti-scarring agent.

1837. The device of item 1589 wherein the device comprises about 10 μg to about 10 mg of the anti-scarring agent.

1838. The device of item 1589 wherein the device comprises about 10 mg to about 250 mg of the anti-scarring agent.

1839. The device of item 1589 wherein the device comprises about 250 mg to about 1000 mg of the anti-scarring agent.

1840. The device of item 1589 wherein the device comprises about 1000 mg to about 2500 mg of the anti-scarring agent.

1841. The device of item 1589 wherein a surface of the device comprises less than 0.01 μg of the anti-scarring agent per mm2 of device surface to which the anti-scarring agent is applied.

1842. The device of item 1589 wherein a surface of the device comprises about 0.01 μg to about 1 μg of the anti-scarring agent per mm2 of device surface to which the anti-scarring agent is applied.

1843. The device of item 1589 wherein a surface of the device comprises about 1 μg to about 10 μg of the anti-scarring agent per mm2 of device surface to which the anti-scarring agent is applied.

1844. The device of item 1589 wherein a surface of the device comprises about 10 μg to about 250 μg of the anti-scarring agent per mm2 of device surface to which the anti-scarring agent is applied.

1845. The device of item 1589 wherein a surface of the device comprises about 250 μg to about 1000 μg of the anti-scarring agent of anti-scarring agent per mm2 of device surface to which the anti-scarring agent is applied.

1846. The device of item 1589 wherein a surface of the device comprises about 1000 μg to about 2500 μg of the anti-scarring agent per mm2 of device surface to which the anti-scarring agent is applied.

1847. A device, comprising a peritoneal dialysis catheter implant and an anti-scarring agent or a composition comprising an anti-scarring agent, wherein the agent inhibits scarring between the device and a host into which the device is implanted.

1848. The device of item 1847 wherein the agent inhibits cell regeneration.

1849. The device of item 1847 wherein the agent inhibits angiogenesis.

1850. The device of item 1847 wherein the agent inhibits fibroblast migration.

1851. The device of item 1847 wherein the agent inhibits fibroblast proliferation.

1852. The device of item 1847 wherein the agent inhibits deposition of extracellular matrix.

1853. The device of item 1847 wherein the agent inhibits tissue remodeling.

1854. The device of item 1847 wherein the agent is an angiogenesis inhibitor.

1855. The device of item 1847 wherein the agent is a 5-lipoxygenase inhibitor or antagonist.

1856. The device of item 1847 wherein the agent is a chemokine receptor antagonist.

1857. The device of item 1847 wherein the agent is a cell cycle inhibitor.

1858. The device of item 1847 wherein the agent is a taxane.

1859. The device of item 1847 wherein the agent is an anti-microtubule agent.

1860. The device of item 1847 wherein the agent is paclitaxel.

1861. The device of item 1847 wherein the agent is not paclitaxel.

1862. The device of item 1847 wherein the agent is an analogue or derivative of paclitaxel.

1863. The device of item 1847 wherein the agent is a vinca alkaloid.

1864. The device of item 1847 wherein the agent is camptothecin or an analogue or derivative thereof.

1865. The device of item 1847 wherein the agent is a podophyllotoxin.

1866. The device of item 1847 wherein the agent is a podophyllotoxin, wherein the podophyllotoxin is etoposide or an analogue or derivative thereof.

1867. The device of item 1847 wherein the agent is an anthracycline.

1868. The device of item 1847 wherein the agent is an anthracycline, wherein the anthracycline is doxorubicin or an analogue or derivative thereof.

1869. The device of item 1847 wherein the agent is an anthracycline, wherein the anthracycline is mitoxantrone or an analogue or derivative thereof.

1870. The device of item 1847 wherein the agent is a platinum compound.

1871. The device of item 1847 wherein the agent is a nitrosourea.

1872. The device of item 1847 wherein the agent is a nitroimidazole.

1873. The device of item 1847 wherein the agent is a folic acid antagonist.

1874. The device of item 1847 wherein the agent is a cytidine analogue.

1875. The device of item 1847 wherein the agent is a pyrimidine analogue.

1876. The device of item 1847 wherein the agent is a fluoropyrimidine analogue.

1877. The device of item 1847 wherein the agent is a purine analogue.

1878. The device of item 1847 wherein the agent is a nitrogen mustard or an analogue or derivative thereof.

1879. The device of item 1847 wherein the agent is a hydroxyurea.

1880. The device of item 1847 wherein the agent is a mytomicin or an analogue or derivative thereof.

1881. The device of item 1847 wherein the agent is an alkyl sulfonate.

1882. The device of item 1847 wherein the agent is a benzamide or an analogue or derivative thereof.

1883. The device of item 1847 wherein the agent is a nicotinamide or an analogue or derivative thereof.

1884. The device of item 1847 wherein the agent is a halogenated sugar or an analogue or derivative thereof.

1885. The device of item 1847 wherein the agent is a DNA alkylating agent.

1886. The device of item 1847 wherein the agent is an anti-microtubule agent.

1887. The device of item 1847 wherein the agent is a topoisomerase inhibitor.

1888. The device of item 1847 wherein the agent is a DNA cleaving agent.

1889. The device of item 1847 wherein the agent is an antimetabolite.

1890. The device of item 1847 wherein the agent inhibits adenosine deaminase.

1891. The device of item 1847 wherein the agent inhibits purine ring synthesis.

1892. The device of item 1847 wherein the agent is a nucleotide interconversion inhibitor.

1893. The device of item 1847 wherein the agent inhibits dihydrofolate reduction.

1894. The device of item 1847 wherein the agent blocks thymidine monophosphate.

1895. The device of item 1847 wherein the agent causes DNA damage.

1896. The device of item 1847 wherein the agent is a DNA intercalation agent.

1897. The device of item 1847 wherein the agent is a RNA synthesis inhibitor.

1898. The device of item 1847 wherein the agent is a pyrimidine synthesis inhibitor.

1899. The device of item 1847 wherein the agent inhibits ribonucleotide synthesis or function.

1900. The device of item 1847 wherein the agent inhibits thymidine monophosphate synthesis or function.

1901. The device of item 1847 wherein the agent inhibits DNA synthesis.

1902. The device of item 1847 wherein the agent causes DNA adduct formation.

1903. The device of item 1847 wherein the agent inhibits protein synthesis.

1904. The device of item 1847 wherein the agent inhibits microtubule function.

1905. The device of item 1847 wherein the agent is a cyclin dependent protein kinase inhibitor.

1906. The device of item 1847 wherein the agent is an epidermal growth factor kinase inhibitor.

1907. The device of item 1847 wherein the agent is an elastase inhibitor.

1908. The device of item 1847 wherein the agent is a factor Xa inhibitor.

1909. The device of item 1847 wherein the agent is a farnesyltransferase inhibitor.

1910. The device of item 1847 wherein the agent is a fibrinogen antagonist.

1911. The device of item 1847 wherein the agent is a guanylate cyclase stimulant.

1912. The device of item 1847 wherein the agent is a heat shock protein 90 antagonist.

1913. The device of item 1847 wherein the agent is a heat shock protein 90 antagonist, wherein the heat shock protein 90 antagonist is geldanamycin or an analogue or derivative thereof.

1914. The device of item 1847 wherein the agent is a guanylate cyclase stimulant.

1915. The device of item 1847 wherein the agent is a HMGCoA reductase inhibitor.

1916. The device of item 1847 wherein the agent is a HMGCoA reductase inhibitor, wherein the HMGCoA reductase inhibitor is simvastatin or an analogue or derivative thereof.

1917. The device of item 1847 wherein the agent is a hydroorotate dehydrogenase inhibitor.

1918. The device of item 1847 wherein the agent is an IKK2 inhibitor.

1919. The device of item 1847 wherein the agent is an IL-1 antagonist.

1920. The device of item 1847 wherein the agent is an ICE antagonist.

1921. The device of item 1847 wherein the agent is an IRAK antagonist.

1922. The device of item 1847 wherein the agent is an IL-4 agonist.

1923. The device of item 1847 wherein the agent is an immunomodulatory agent.

1924. The device of item 1847 wherein the agent is sirolimus or an analogue or derivative thereof.

1925. The device of item 1847 wherein the agent is not sirolimus.

1926. The device of item 1847 wherein the agent is everolimus or an analogue or derivative thereof.

1927. The device of item 1847 wherein the agent is tacrolimus or an analogue or derivative thereof.

1928. The device of item 1847 wherein the agent is not tacrolimus.

1929. The device of item 1847 wherein the agent is biolmus or an analogue or derivative thereof.

1930. The device of item 1847 wherein the agent is tresperimus or an analogue or derivative thereof.

1931. The device of item 1847 wherein the agent is auranofin or an analogue or derivative thereof.

1932. The device of item 1847 wherein the agent is 27-0-demethylrapamycin or an analogue or derivative thereof.

1933. The device of item 1847 wherein the agent is gusperimus or an analogue or derivative thereof.

1934. The device of item 1847 wherein the agent is pimecrolimus or an analogue or derivative thereof.

1935. The device of item 1847 wherein the agent is ABT-578 or an analogue or derivative thereof.

1936. The device of item 1847 wherein the agent is an inosine monophosphate dehydrogenase (IMPDH) inhibitor.

1937. The device of item 1847 wherein the agent is an IMPDH inhibitor, wherein the IMPDH inhibitor is mycophenolic acid or an analogue or derivative thereof.

1938. The device of item 1847 wherein the agent is an IMPDH inhibitor, wherein the IMPDH inhibitor is 1-alpha-25 dihydroxy vitamin D3 or an analogue or derivative thereof.

1939. The device of item 1847 wherein the agent is a leukotriene inhibitor.

1940. The device of item 1847 wherein the agent is a MCP-1 antagonist.

1941. The device of item 1847 wherein the agent is a MMP inhibitor.

1942. The device of item 1847 wherein the agent is an NF kappa B inhibitor.

1943. The device of item 1847 wherein the agent is an NF kappa B inhibitor, wherein the NF kappa B inhibitor is Bay 11-7082.

1944. The device of item 1847 wherein the agent is an NO agonist.

1945. The device of item 1847 wherein the agent is a p38 MAP kinase inhibitor.

1946. The device of item 1847 wherein the agent is a p38 MAP kinase inhibitor, wherein the p38 MAP kinase inhibitor is SB 202190.

1947. The device of item 1847 wherein the agent is a phosphodiesterase inhibitor.

1948. The device of item 1847 wherein the agent is a TGF beta inhibitor.

1949. The device of item 1847 wherein the agent is a thromboxane A2 antagonist.

1950. The device of item 1847 wherein the agent is a TNFa antagonist.

1951. The device of item 1847 wherein the agent is a TACE inhibitor.

1952. The device of item 1847 wherein the agent is a tyrosine kinase inhibitor.

1953. The device of item 1847 wherein the agent is a vitronectin inhibitor.

1954. The device of item 1847 wherein the agent is a fibroblast growth factor inhibitor.

1955. The device of item 1847 wherein the agent is a protein kinase inhibitor.

1956. The device of item 1847 wherein the agent is a PDGF receptor kinase inhibitor.

1957. The device of item 1847 wherein the agent is an endothelial growth factor receptor kinase inhibitor.

1958. The device of item 1847 wherein the agent is a retinoic acid receptor antagonist.

1959. The device of item 1847 wherein the agent is a platelet derived growth factor receptor kinase inhibitor.

1960. The device of item 1847 wherein the agent is a fibronogin antagonist.

1961. The device of item 1847 wherein the agent is an antimycotic agent.

1962. The device of item 1847 wherein the agent is an antimycotic agent, wherein the antimycotic agent is sulconizole.

1963. The device of item 1847 wherein the agent is a bisphosphonate.

1964. The device of item 1847 wherein the agent is a phospholipase A1 inhibitor.

1965. The device of item 1847 wherein the agent is a histamine H1/H2/H3 receptor antagonist.

1966. The device of item 1847 wherein the agent is a macrolide antibiotic.

1967. The device of item 1847 wherein the agent is a GPIIb/IIIa receptor antagonist.

1968. The device of item 1847 wherein the agent is an endothelin receptor antagonist.

1969. The device of item 1847 wherein the agent is a peroxisome proliferator-activated receptor agonist.

1970. The device of item 1847 wherein the agent is an estrogen receptor agent.

1971. The device of item 1847 wherein the agent is a somastostatin analogue.

1972. The device of item 1847 wherein the agent is a neurokinin 1 antagonist.

1973. The device of item 1847 wherein the agent is a neurokinin 3 antagonist.

1974. The device of item 1847 wherein the agent is a VLA-4 antagonist.

1975. The device of item 1847 wherein the agent is an osteoclast inhibitor.

1976. The device of item 1847 wherein the agent is a DNA topoisomerase ATP hydrolyzing inhibitor.

1977. The device of item 1847 wherein the agent is an angiotensin I converting enzyme inhibitor.

1978. The device of item 1847 wherein the agent is an angiotensin II antagonist.

1979. The device of item 1847 wherein the agent is an enkephalinase inhibitor.

1980. The device of item 1847 wherein the agent is a peroxisome proliferator-activated receptor gamma agonist insulin sensitizer.

1981. The device of item 1847 wherein the agent is a protein kinase C inhibitor.

1982. The device of item 1847 wherein the agent is a ROCK (rho-associated kinase) inhibitor.

1983. The device of item 1847 wherein the agent is a CXCR3 inhibitor.

1984. The device of item 1847 wherein the agent is an Itk inhibitor.

1985. The device of item 1847 wherein the agent is a cytosolic phospholipase A2-alpha inhibitor.

1986. The device of item 1847 wherein the agent is a PPAR agonist.

1987. The device of item 1847 wherein the agent is an immunosuppressant.

1988. The device of item 1847 wherein the agent is an Erb inhibitor.

1989. The device of item 1847 wherein the agent is an apoptosis agonist.

1990. The device of item 1847 wherein the agent is a lipocortin agonist.

1991. The device of item 1847 wherein the agent is a VCAM-1 antagonist.

1992. The device of item 1847 wherein the agent is a collagen antagonist.

1993. The device of item 1847 wherein the agent is an alpha 2 integrin antagonist.

1994. The device of item 1847 wherein the agent is a TNF alpha inhibitor.

1995. The device of item 1847 wherein the agent is a nitric oxide inhibitor 1996. The device of item 1847 wherein the agent is a cathepsin inhibitor.

1997. The device of item 1847 wherein the agent is not an anti-inflammatory agent.

1998. The device of item 1847 wherein the agent is not a steroid.

1999. The device of item 1847 wherein the agent is not a glucocorticosteroid.

2000. The device of item 1847 wherein the agent is not dexamethasone.

2001. The device of item 1847 wherein the agent is not an anti-infective agent.

2002. The device of item 1847 wherein the agent is not an antibiotic.

2003. The device of item 1847 wherein the agent is not an anti-fungal agent.

2004. The device of item 1847, further comprising a polymer.

2005. The device of item 1847, further comprising a polymeric carrier.

2006. The device of item 1847 wherein the anti-scarring agent inhibits adhesion between the device and a host into which the device is implanted.

2007. The device of item 1847 wherein the device delivers the anti-scarring agent locally to tissue proximate to the device.

2008. The device of item 1847, further comprising a coating, wherein the coating comprises the anti-scarring agent.

2009. The device of item 1847, further comprising a coating, wherein the coating is disposed on a surface of the device.

2010. The device of item 1847, further comprising a coating, wherein the coating directly contacts the device.

2011. The device of item 1847, further comprising a coating, wherein the coating indirectly contacts the device.

2012. The device of item 1847, further comprising a coating, wherein the coating partially covers the device.

2013. The device of item 1847, further comprising a coating, wherein the coating completely covers the device.

2014. The device of item 1847, further comprising a coating, wherein the coating is a uniform coating.

2015. The device of item 1847, further comprising a coating, wherein the coating is a non-uniform coating.

2016. The device of item 1847, further comprising a coating, wherein the coating is a discontinuous coating.

2017. The device of item 1847, further comprising a coating, wherein the coating is a patterned coating.

2018. The device of item 1847, further comprising a coating, wherein the coating has a thickness of 100 μm or less.

2019. The device of item 1847, further comprising a coating, wherein the coating has a thickness of 10 μm or less.

2020. The device of item 1847, further comprising a coating, wherein the coating adheres to the surface of the device upon deployment of the device.

2021. The device of item 1847, further comprising a coating, wherein the coating is stable at room temperature for a period of 1 year.

2022. The device of item 1847, further comprising a coating, wherein the anti-scarring agent is present in the coating in an amount ranging between about 0.0001% to about 1% by weight.

2023. The device of item 1847, further comprising a coating, wherein the anti-scarring agent is present in the coating in an amount ranging between about 1% to about 10% by weight.

2024. The device of item 1847, further comprising a coating, wherein the anti-scarring agent is present in the coating in an amount ranging between about 10% to about 25% by weight.

2025. The device of item 1847, further comprising a coating, wherein the anti-scarring agent is present in the coating in an amount ranging between about 25% to about 70% by weight.

2026. The device of item 1847, further comprising a coating, wherein the coating further comprises a polymer.

2027. The device of item 1847, further comprising a first coating having a first composition and the second coating having a second composition.

2028. The device of item 1847, further comprising a first coating having a first composition and the second coating having a second composition, wherein the first composition and the second composition are different.

2029. The device of item 1847, further comprising a polymer.

2030. The device of item 1847, further comprising a polymeric carrier.

2031. The device of item 1847, further comprising a polymeric carrier, wherein the polymeric carrier comprises a copolymer.

2032. The device of item 1847, further comprising a polymeric carrier, wherein the polymeric carrier comprises a block copolymer.

2033. The device of item 1847, further comprising a polymeric carrier, wherein the polymeric carrier comprises a random copolymer.

2034. The device of item 1847, further comprising a polymeric carrier, wherein the polymeric carrier comprises a biodegradable polymer.

2035. The device of item 1847, further comprising a polymeric carrier, wherein the polymeric carrier comprises a non-biodegradable polymer.

2036. The device of item 1847, further comprising a polymeric carrier, wherein the polymeric carrier comprises a hydrophilic polymer.

2037. The device of item 1847, further comprising a polymeric carrier, wherein the polymeric carrier comprises a hydrophobic polymer.

2038. The device of item 1847, further comprising a polymeric carrier, wherein the polymeric carrier comprises a polymer having hydrophilic domains.

2039. The device of item 1847, further comprising a polymeric carrier, wherein the polymeric carrier comprises a polymer having hydrophobic domains.

2040. The device of item 1847, further comprising a polymeric carrier, wherein the polymeric carrier comprises a non-conductive polymer.

2041. The device of item 1847, further comprising a polymeric carrier, wherein the polymeric carrier comprises an elastomer.

2042. The device of item 1847, further comprising a polymeric carrier, wherein the polymeric carrier comprises a hydrogel.

2043. The device of item 1847, further comprising a polymeric carrier, wherein the polymeric carrier comprises a silicone polymer.

2044. The device of item 1847, further comprising a polymeric carrier, wherein the polymeric carrier comprises a hydrocarbon polymer.

2045. The device of item 1847, further comprising a polymeric carrier, wherein the polymeric carrier comprises a styrene-derived polymer.

2046. The device of item 1847, further comprising a polymeric carrier, wherein the polymeric carrier comprises a butadiene polymer.

2047. The device of item 1847, further comprising a polymeric carrier, wherein the polymeric carrier comprises a macromer.

2048. The device of item 1847, further comprising a polymeric carrier, wherein the polymeric carrier comprises a poly(ethylene glycol)polymer.

2049. The device of item 1847, further comprising a polymeric carrier, wherein the polymeric carrier comprises an amorphous polymer.

2050. The device of item 1847, further comprising a lubricious coating.

2051. The device of item 1847 wherein the anti-scarring agent is located within pores or holes of the device.

2052. The device of item 1847 wherein the anti-scarring agent is located within a channel, lumen, or divet of the device.

2053. The device of item 1847, further comprising a second pharmaceutically active agent.

2054. The device of item 1847, further comprising an anti-inflammatory agent.

2055. The device of item 1847, further comprising an agent that inhibits infection.

2056. The device of item 1847, further comprising an agent that inhibits infection, wherein the agent is an anthracycline.

2057. The device of item 1847, further comprising an agent that inhibits infection, wherein the agent is doxorubicin.

2058. The device of item 1847, further comprising an agent that inhibits infection, wherein the agent is mitoxantrone.

2059. The device of item 1847, further comprising an agent that inhibits infection, wherein the agent is a fluoropyrimidine.

2060. The device of item 1847, further comprising an agent that inhibits infection, wherein the agent is 5-fluorouracil (5-FU).

2061. The device of item 1847, further comprising an agent that inhibits infection, wherein the agent is a folic acid antagonist.

2062. The device of item 1847, further comprising an agent that inhibits infection, wherein the agent is methotrexate.

2063. The device of item 1847, further comprising an agent that inhibits infection, wherein the agent is a podophylotoxin.

2064. The device of item 1847, further comprising an agent that inhibits infection, wherein the agent is etoposide.

2065. The device of item 1847, further comprising an agent that inhibits infection, wherein the agent is a camptothecin.

2066. The device of item 1847, further comprising an agent that inhibits infection, wherein the agent is a hydroxyurea.

2067. The device of item 1847, further comprising an agent that inhibits infection, wherein the agent is a platinum complex.

2068. The device of item 1847, further comprising an agent that inhibits infection, wherein the agent is cisplatin.

2069. The device of item 1847, further comprising an anti-thrombotic agent.

2070. The device of item 1847, further comprising a visualization agent.

2071. The device of item 1847, further comprising a visualization agent, wherein the visualization agent is a radiopaque material, wherein the radiopaque material comprises a metal, a halogenated compound, or a barium containing compound.

2072. The device of item 1847, further comprising a visualization agent, wherein the visualization agent is a radiopaque material, wherein the radiopaque material comprises barium, tantalum, or technetium.

2073. The device of item 1847, further comprising a visualization agent, wherein the visualization agent is a MRI responsive material.

2074. The device of item 1847, further comprising a visualization agent, wherein the visualization agent comprises a gadolinium chelate.

2075. The device of item 1847, further comprising a visualization agent, wherein the visualization agent comprises iron, magnesium, manganese, copper, or chromium.

2076. The device of item 1847, further comprising a visualization agent, wherein the visualization agent comprises an iron oxide compound.

2077. The device of item 1847, further comprising a visualization agent, wherein the visualization agent comprises a dye, pigment, or colorant.

2078. The device of item 1847, further comprising an echogenic material.

2079. The device of item 1847, further comprising an echogenic material, wherein the echogenic material is in the form of a coating.

2080. The device of item 1847 wherein the device is sterile.

2081. The device of item 1847 wherein the anti-scarring agent is released into tissue in the vicinity of the device after deployment of the device.

2082. The device of item 1847 wherein the anti-scarring agent is released into tissue in the vicinity of the device after deployment of the device, wherein the tissue is connective tissue.

2083. The device of item 1847 wherein the anti-scarring agent is released into tissue in the vicinity of the device after deployment of the device, wherein the tissue is muscle tissue.

2084. The device of item 1847 wherein the anti-scarring agent is released into tissue in the vicinity of the device after deployment of the device, wherein the tissue is nerve tissue.

2085. The device of item 1847 wherein the anti-scarring agent is released into tissue in the vicinity of the device after deployment of the device, wherein the tissue is epithelium tissue.

2086. The device of item 1847 wherein the anti-scarring agent is released in effective concentrations from the device over a period ranging from the time of deployment of the device to about 1 year.

2087. The device of item 1847 wherein the anti-scarring agent is released in effective concentrations from the device over a period ranging from about 1 month to 6 months.

2088. The device of item 1847 wherein the anti-scarring agent is released in effective concentrations from the device over a period ranging from about 1-90 days.

2089. The device of item 1847 wherein the anti-scarring agent is released in effective concentrations from the device at a constant rate.

2090. The device of item 1847 wherein the anti-scarring agent is released in effective concentrations from the device at an increasing rate.

2091. The device of item 1847 wherein the anti-scarring agent is released in effective concentrations from the device at a decreasing rate.

2092. The device of item 1847 wherein the anti-scarring agent is released in effective concentrations from the composition comprising the anti-scarring agent by diffusion over a period ranging from the time of deployment of the device to about 90 days.

2093. The device of item 1847 wherein the anti-scarring agent is released in effective concentrations from the composition comprising the anti-scarring agent by erosion of the composition over a period ranging from the time of deployment of the device to about 90 days.

2094. The device of item 1847 wherein the device comprises about 0.01 μg to about 10 μg of the anti-scarring agent.

2095. The device of item 1847 wherein the device comprises about 10 μg to about 10 mg of the anti-scarring agent.

2096. The device of item 1847 wherein the device comprises about 10 mg to about 250 mg of the anti-scarring agent.

2097. The device of item 1847 wherein the device comprises about 250 mg to about 1000 mg of the anti-scarring agent.

2098. The device of item 1847 wherein the device comprises about 1000 mg to about 2500 mg of the anti-scarring agent.

2099. The device of item 1847 wherein a surface of the device comprises less than 0.01 μg of the anti-scarring agent per mm2 of device surface to which the anti-scarring agent is applied.

2100. The device of item 1847 wherein a surface of the device comprises about 0.01 μg to about 1 μg of the anti-scarring agent per mm2 of device surface to which the anti-scarring agent is applied.

2101. The device of item 1847 wherein a surface of the device comprises about 1 μg to about 10 μg of the anti-scarring agent per mm2 of device surface to which the anti-scarring agent is applied.

2102. The device of item 1847 wherein a surface of the device comprises about 10 μg to about 250 μg of the anti-scarring agent per mm2 of device surface to which the anti-scarring agent is applied.

2103. The device of item 1847 wherein a surface of the device comprises about 250 μg to about 1000 μg of the anti-scarring agent of anti-scarring agent per mm2 of device surface to which the anti-scarring agent is applied.

2104. The device of item 1847 wherein a surface of the device comprises about 1000 μg to about 2500 μg of the anti-scarring agent per mm2 of device surface to which the anti-scarring agent is applied.

2105. A device, comprising a central nervous system shunt (i.e., an implant) and an anti-scarring agent or a composition comprising an anti-scarring agent, wherein the agent inhibits scarring between the device and a host into which the device is implanted.

2106. The device of item 2105 wherein the agent inhibits cell regeneration.

2107. The device of item 2105 wherein the agent inhibits angiogenesis.

2108. The device of item 2105 wherein the agent inhibits fibroblast migration.

2109. The device of item 2105 wherein the agent inhibits fibroblast proliferation.

2110. The device of item 2105 wherein the agent inhibits deposition of extracellular matrix.

2111. The device of item 2105 wherein the agent inhibits tissue remodeling.

2112. The device of item 2105 wherein the agent is an angiogenesis inhibitor.

2113. The device of item 2105 wherein the agent is a 5-lipoxygenase inhibitor or antagonist.

2114. The device of item 2105 wherein the agent is a chemokine receptor antagonist.

2115. The device of item 2105 wherein the agent is a cell cycle inhibitor.

2116. The device of item 2105 wherein the agent is a taxane.

2117. The device of item 2105 wherein the agent is an anti-microtubule agent.

2118. The device of item 2105 wherein the agent is paclitaxel.

2119. The device of item 2105 wherein the agent is not paclitaxel.

2120. The device of item 2105 wherein the agent is an analogue or derivative of paclitaxel.

2121. The device of item 2105 wherein the agent is a vinca alkaloid.

2122. The device of item 2105 wherein the agent is camptothecin or an analogue or derivative thereof.

2123. The device of item 2105 wherein the agent is a podophyllotoxin.

2124. The device of item 2105 wherein the agent is a podophyllotoxin, wherein the podophyllotoxin is etoposide or an analogue or derivative thereof.

2125. The device of item 2105 wherein the agent is an anthracycline.

2126. The device of item 2105 wherein the agent is an anthracycline, wherein the anthracycline is doxorubicin or an analogue or derivative thereof.

2127. The device of item 2105 wherein the agent is an anthracycline, wherein the anthracycline is mitoxantrone or an analogue or derivative thereof.

2128. The device of item 2105 wherein the agent is a platinum compound.

2129. The device of item 2105 wherein the agent is a nitrosourea.

2130. The device of item 2105 wherein the agent is a nitroimidazole.

2131. The device of item 2105 wherein the agent is a folic acid antagonist.

2132. The device of item 2105 wherein the agent is a cytidine analogue.

2133. The device of item 2105 wherein the agent is a pyrimidine analogue.

2134. The device of item 2105 wherein the agent is a fluoropyrimidine analogue.

2135. The device of item 2105 wherein the agent is a purine analogue.

2136. The device of item 2105 wherein the agent is a nitrogen mustard or an analogue or derivative thereof.

2137. The device of item 2105 wherein the agent is a hydroxyurea.

2138. The device of item 2105 wherein the agent is a mytomicin or an analogue or derivative thereof.

2139. The device of item 2105 wherein the agent is an alkyl sulfonate.

2140. The device of item 2105 wherein the agent is a benzamide or an analogue or derivative thereof.

2141. The device of item 2105 wherein the agent is a nicotinamide or an analogue or derivative thereof.

2142. The device of item 2105 wherein the agent is a halogenated sugar or an analogue or derivative thereof.

2143. The device of item 2105 wherein the agent is a DNA alkylating agent.

2144. The device of item 2105 wherein the agent is an anti-microtubule agent.

2145. The device of item 2105 wherein the agent is a topoisomerase inhibitor.

2146. The device of item 2105 wherein the agent is a DNA cleaving agent.

2147. The device of item 2105 wherein the agent is an antimetabolite.

2148. The device of item 2105 wherein the agent inhibits adenosine deaminase.

2149. The device of item 2105 wherein the agent inhibits purine ring synthesis.

2150. The device of item 2105 wherein the agent is a nucleotide interconversion inhibitor.

2151. The device of item 2105 wherein the agent inhibits dihydrofolate reduction.

2152. The device of item 2105 wherein the agent blocks thymidine monophosphate.

2153. The device of item 2105 wherein the agent causes DNA damage.

2154. The device of item 2105 wherein the agent is a DNA intercalation agent.

2155. The device of item 2105 wherein the agent is a RNA synthesis inhibitor.

2156. The device of item 2105 wherein the agent is a pyrimidine synthesis inhibitor.

2157. The device of item 2105 wherein the agent inhibits ribonucleotide synthesis or function.

2158. The device of item 2105 wherein the agent inhibits thymidine monophosphate synthesis or function.

2159. The device of item 2105 wherein the agent inhibits DNA synthesis.

2160. The device of item 2105 wherein the agent causes DNA adduct formation.

2161. The device of item 2105 wherein the agent inhibits protein synthesis.

2162. The device of item 2105 wherein the agent inhibits microtubule function.

2163. The device of item 2105 wherein the agent is a cyclin dependent protein kinase inhibitor.

2164. The device of item 2105 wherein the agent is an epidermal growth factor kinase inhibitor.

2165. The device of item 2105 wherein the agent is an elastase inhibitor.

2166. The device of item 2105 wherein the agent is a factor Xa inhibitor.

2167. The device of item 2105 wherein the agent is a farnesyltransferase inhibitor.

2168. The device of item 2105 wherein the agent is a fibrinogen antagonist.

2169. The device of item 2105 wherein the agent is a guanylate cyclase stimulant.

2170. The device of item 2105 wherein the agent is a heat shock protein 90 antagonist.

2171. The device of item 2105 wherein the agent is a heat shock protein 90 antagonist, wherein the heat shock protein 90 antagonist is geldanamycin or an analogue or derivative thereof.

2172. The device of item 2105 wherein the agent is a guanylate cyclase stimulant.

2173. The device of item 2105 wherein the agent is a HMGCoA reductase inhibitor.

2174. The device of item 2105 wherein the agent is a HMGCoA reductase inhibitor, wherein the HMGCoA reductase inhibitor is simvastatin or an analogue or derivative thereof.

2175. The device of item 2105 wherein the agent is a hydroorotate dehydrogenase inhibitor.

2176. The device of item 2105 wherein the agent is an IKK2 inhibitor.

2177. The device of item 2105 wherein the agent is an IL-1 antagonist.

2178. The device of item 2105 wherein the agent is an ICE antagonist.

2179. The device of item 2105 wherein the agent is an IRAK antagonist.

2180. The device of item 2105 wherein the agent is an IL-4 agonist.

2181. The device of item 2105 wherein the agent is an immunomodulatory agent.

2182. The device of item 2105 wherein the agent is sirolimus or an analogue or derivative thereof.

2183. The device of item 2105 wherein the agent is not sirolimus.

2184. The device of item 2105 wherein the agent is everolimus or an analogue or derivative thereof.

2185. The device of item 2105 wherein the agent is tacrolimus or an analogue or derivative thereof.

2186. The device of item 2105 wherein the agent is not tacrolimus.

2187. The device of item 2105 wherein the agent is biolmus or an analogue or derivative thereof.

2188. The device of item 2105 wherein the agent is tresperimus or an analogue or derivative thereof.

2189. The device of item 2105 wherein the agent is auranofin or an analogue or derivative thereof.

2190. The device of item 2105 wherein the agent is 27-0-demethylrapamycin or an analogue or derivative thereof.

2191. The device of item 2105 wherein the agent is gusperimus or an analogue or derivative thereof.

2192. The device of item 2105 wherein the agent is pimecrolimus or an analogue or derivative thereof.

2193. The device of item 2105 wherein the agent is ABT-578 or an analogue or derivative thereof.

2194. The device of item 2105 wherein the agent is an inosine monophosphate dehydrogenase (IMPDH) inhibitor.

2195. The device of item 2105 wherein the agent is an IMPDH inhibitor, wherein the IMPDH inhibitor is mycophenolic acid or an analogue or derivative thereof.

2196. The device of item 2105 wherein the agent is an IMPDH inhibitor, wherein the IMPDH inhibitor is 1-alpha-25 dihydroxy vitamin D3 or an analogue or derivative thereof.

2197. The device of item 2105 wherein the agent is a leukotriene inhibitor.

2198. The device of item 2105 wherein the agent is a MCP-1 antagonist.

2199. The device of item 2105 wherein the agent is a MMP inhibitor.

2200. The device of item 2105 wherein the agent is an NF kappa B inhibitor.

2201. The device of item 2105 wherein the agent is an NF kappa B inhibitor, wherein the NF kappa B inhibitor is Bay 11-7082.

2202. The device of item 2105 wherein the agent is an NO agonist.

2203. The device of item 2105 wherein the agent is a p38 MAP kinase inhibitor.

2204. The device of item 2105 wherein the agent is a p38 MAP kinase inhibitor, wherein the p38 MAP kinase inhibitor is SB 202190.

2205. The device of item 2105 wherein the agent is a phosphodiesterase inhibitor.

2206. The device of item 2105 wherein the agent is a TGF beta inhibitor.

2207. The device of item 2105 wherein the agent is a thromboxane A2 antagonist.

2208. The device of item 2105 wherein the agent is a TNFa antagonist.

2209. The device of item 2105 wherein the agent is a TACE inhibitor.

2210. The device of item 2105 wherein the agent is a tyrosine kinase inhibitor.

2211. The device of item 2105 wherein the agent is a vitronectin inhibitor.

2212. The device of item 2105 wherein the agent is a fibroblast growth factor inhibitor.

2213. The device of item 2105 wherein the agent is a protein kinase inhibitor.

2214. The device of item 2105 wherein the agent is a PDGF receptor kinase inhibitor.

2215. The device of item 2105 wherein the agent is an endothelial growth factor receptor kinase inhibitor.

2216. The device of item 2105 wherein the agent is a retinoic acid receptor antagonist.

2217. The device of item 2105 wherein the agent is a platelet derived growth factor receptor kinase inhibitor.

2218. The device of item 2105 wherein the agent is a fibronogin antagonist.

2219. The device of item 2105 wherein the agent is an antimycotic agent.

2220. The device of item 2105 wherein the agent is an antimycotic agent, wherein the antimycotic agent is sulconizole.

2221. The device of item 2105 wherein the agent is a bisphosphonate.

2222. The device of item 2105 wherein the agent is a phospholipase A1 inhibitor.

2223. The device of item 2105 wherein the agent is a histamine H1/H2/H3 receptor antagonist.

2224. The device of item 2105 wherein the agent is a macrolide antibiotic.

2225. The device of item 2105 wherein the agent is a GPIIb/IIIa receptor antagonist.

2226. The device of item 2105 wherein the agent is an endothelin receptor antagonist.

2227. The device of item 2105 wherein the agent is a peroxisome proliferator-activated receptor agonist.

2228. The device of item 2105 wherein the agent is an estrogen receptor agent.

2229. The device of item 2105 wherein the agent is a somastostatin analogue.

2230. The device of item 2105 wherein the agent is a neurokinin 1 antagonist.

2231. The device of item 2105 wherein the agent is a neurokinin 3 antagonist.

2232. The device of item 2105 wherein the agent is a VLA-4 antagonist.

2233. The device of item 2105 wherein the agent is an osteoclast inhibitor.

2234. The device of item 2105 wherein the agent is a DNA topoisomerase ATP hydrolyzing inhibitor.

2235. The device of item 2105 wherein the agent is an angiotensin I converting enzyme inhibitor.

2236. The device of item 2105 wherein the agent is an angiotensin II antagonist.

2237. The device of item 2105 wherein the agent is an enkephalinase inhibitor.

2238. The device of item 2105 wherein the agent is a peroxisome proliferator-activated receptor gamma agonist insulin sensitizer.

2239. The device of item 2105 wherein the agent is a protein kinase C inhibitor.

2240. The device of item 2105 wherein the agent is a ROCK (rho-associated kinase) inhibitor.

2241. The device of item 2105 wherein the agent is a CXCR3 inhibitor.

2242. The device of item 2105 wherein the agent is an Itk inhibitor.

2243. The device of item 2105 wherein the agent is a cytosolic phospholipase A2-alpha inhibitor.

2244. The device of item 2105 wherein the agent is a PPAR agonist.

2245. The device of item 2105 wherein the agent is an immunosuppressant.

2246. The device of item 2105 wherein the agent is an Erb inhibitor.

2247. The device of item 2105 wherein the agent is an apoptosis agonist.

2248. The device of item 2105 wherein the agent is a lipocortin agonist.

2249. The device of item 2105 wherein the agent is a VCAM-1 antagonist.

2250. The device of item 2105 wherein the agent is a collagen antagonist.

2251. The device of item 2105 wherein the agent is an alpha 2 integrin antagonist.

2252. The device of item 2105 wherein the agent is a TNF alpha inhibitor.

2253. The device of item 2105 wherein the agent is a nitric oxide inhibitor 2254. The device of item 2105 wherein the agent is a cathepsin inhibitor.

2255. The device of item 2105 wherein the agent is not an anti-inflammatory agent.

2256. The device of item 2105 wherein the agent is not a steroid.

2257. The device of item 2105 wherein the agent is not a glucocorticosteroid.

2258. The device of item 2105 wherein the agent is not dexamethasone.

2259. The device of item 2105 wherein the agent is not an anti-infective agent.

2260. The device of item 2105 wherein the agent is not an antibiotic.

2261. The device of item 2105 wherein the agent is not an anti-fungal agent.

2262. The device of item 2105, further comprising a polymer.

2263. The device of item 2105, further comprising a polymeric carrier.

2264. The device of item 2105 wherein the anti-scarring agent inhibits adhesion between the device and a host into which the device is implanted.

2265. The device of item 2105 wherein the device delivers the anti-scarring agent locally to tissue proximate to the device.

2266. The device of item 2105, further comprising a coating, wherein the coating comprises the anti-scarring agent.

2267. The device of item 2105, further comprising a coating, wherein the coating is disposed on a surface of the device.

2268. The device of item 2105, further comprising a coating, wherein the coating directly contacts the device.

2269. The device of item 2105, further comprising a coating, wherein the coating indirectly contacts the device.

2270. The device of item 2105, further comprising a coating, wherein the coating partially covers the device.

2271. The device of item 2105, further comprising a coating, wherein the coating completely covers the device.

2272. The device of item 2105, further comprising a coating, wherein the coating is a uniform coating.

2273. The device of item 2105, further comprising a coating, wherein the coating is a non-uniform coating.

2274. The device of item 2105, further comprising a coating, wherein the coating is a discontinuous coating.

2275. The device of item 2105, further comprising a coating, wherein the coating is a patterned coating.

2276. The device of item 2105, further comprising a coating, wherein the coating has a thickness of 100 μm or less.

2277. The device of item 2105, further comprising a coating, wherein the coating has a thickness of 10 μm or less.

2278. The device of item 2105, further comprising a coating, wherein the coating adheres to the surface of the device upon deployment of the device.

2279. The device of item 2105, further comprising a coating, wherein the coating is stable at room temperature for a period of 1 year.

2280. The device of item 2105, further comprising a coating, wherein the anti-scarring agent is present in the coating in an amount ranging between about 0.0001% to about 1% by weight.

2281. The device of item 2105, further comprising a coating, wherein the anti-scarring agent is present in the coating in an amount ranging between about 1% to about 10% by weight.

2282. The device of item 2105, further comprising a coating, wherein the anti-scarring agent is present in the coating in an amount ranging between about 10% to about 25% by weight.

2283. The device of item 2105, further comprising a coating, wherein the anti-scarring agent is present in the coating in an amount ranging between about 25% to about 70% by weight.

2284. The device of item 2105, further comprising a coating, wherein the coating further comprises a polymer.

2285. The device of item 2105, further comprising a first coating having a first composition and the second coating having a second composition.

2286. The device of item 2105, further comprising a first coating having a first composition and the second coating having a second composition, wherein the first composition and the second composition are different.

2287. The device of item 2105, further comprising a polymer.

2288. The device of item 2105, further comprising a polymeric carrier.

2289. The device of item 2105, further comprising a polymeric carrier, wherein the polymeric carrier comprises a copolymer.

2290. The device of item 2105, further comprising a polymeric carrier, wherein the polymeric carrier comprises a block copolymer.

2291. The device of item 2105, further comprising a polymeric carrier, wherein the polymeric carrier comprises a random copolymer.

2292. The device of item 2105, further comprising a polymeric carrier, wherein the polymeric carrier comprises a biodegradable polymer.

2293. The device of item 2105, further comprising a polymeric carrier, wherein the polymeric carrier comprises a non-biodegradable polymer.

2294. The device of item 2105, further comprising a polymeric carrier, wherein the polymeric carrier comprises a hydrophilic polymer.

2295. The device of item 2105, further comprising a polymeric carrier, wherein the polymeric carrier comprises a hydrophobic polymer.

2296. The device of item 2105, further comprising a polymeric carrier, wherein the polymeric carrier comprises a polymer having hydrophilic domains.

2297. The device of item 2105, further comprising a polymeric carrier, wherein the polymeric carrier comprises a polymer having hydrophobic domains.

2298. The device of item 2105, further comprising a polymeric carrier, wherein the polymeric carrier comprises a non-conductive polymer.

2299. The device of item 2105, further comprising a polymeric carrier, wherein the polymeric carrier comprises an elastomer.

2300. The device of item 2105, further comprising a polymeric carrier, wherein the polymeric carrier comprises a hydrogel.

2301. The device of item 2105, further comprising a polymeric carrier, wherein the polymeric carrier comprises a silicone polymer.

2302. The device of item 2105, further comprising a polymeric carrier, wherein the polymeric carrier comprises a hydrocarbon polymer.

2303. The device of item 2105, further comprising a polymeric carrier, wherein the polymeric carrier comprises a styrene-derived polymer.

2304. The device of item 2105, further comprising a polymeric carrier, wherein the polymeric carrier comprises a butadiene polymer.

2305. The device of item 2105, further comprising a polymeric carrier, wherein the polymeric carrier comprises a macromer.

2306. The device of item 2105, further comprising a polymeric carrier, wherein the polymeric carrier comprises a poly(ethylene glycol)polymer.

2307. The device of item 2105, further comprising a polymeric carrier, wherein the polymeric carrier comprises an amorphous polymer.

2308. The device of item 2105, further comprising a lubricious coating.

2309. The device of item 2105 wherein the anti-scarring agent is located within pores or holes of the device.

2310. The device of item 2105 wherein the anti-scarring agent is located within a channel, lumen, or divet of the device.

2311. The device of item 2105, further comprising a second pharmaceutically active agent.

2312. The device of item 2105, further comprising an anti-inflammatory agent.

2313. The device of item 2105, further comprising an agent that inhibits infection.

2314. The device of item 2105, further comprising an agent that inhibits infection, wherein the agent is an anthracycline.

2315. The device of item 2105, further comprising an agent that inhibits infection, wherein the agent is doxorubicin.

2316. The device of item 2105, further comprising an agent that inhibits infection, wherein the agent is mitoxantrone.

2317. The device of item 2105, further comprising an agent that inhibits infection, wherein the agent is a fluoropyrimidine.

2318. The device of item 2105, further comprising an agent that inhibits infection, wherein the agent is 5-fluorouracil (5-FU).

2319. The device of item 2105, further comprising an agent that inhibits infection, wherein the agent is a folic acid antagonist.

2320. The device of item 2105, further comprising an agent that inhibits infection, wherein the agent is methotrexate.

2321. The device of item 2105, further comprising an agent that inhibits infection, wherein the agent is a podophylotoxin.

2322. The device of item 2105, further comprising an agent that inhibits infection, wherein the agent is etoposide.

2323. The device of item 2105, further comprising an agent that inhibits infection, wherein the agent is a camptothecin.

2324. The device of item 2105, further comprising an agent that inhibits infection, wherein the agent is a hydroxyurea.

2325. The device of item 2105, further comprising an agent that inhibits infection, wherein the agent is a platinum complex.

2326. The device of item 2105, further comprising an agent that inhibits infection, wherein the agent is cisplatin.

2327. The device of item 2105, further comprising an anti-thrombotic agent.

2328. The device of item 2105, further comprising a visualization agent.

2329. The device of item 2105, further comprising a visualization agent, wherein the visualization agent is a radiopaque material, wherein the radiopaque material comprises a metal, a halogenated compound, or a barium containing compound.

2330. The device of item 2105, further comprising a visualization agent, wherein the visualization agent is a radiopaque material, wherein the radiopaque material comprises barium, tantalum, or technetium.

2331. The device of item 2105, further comprising a visualization agent, wherein the visualization agent is a MRI responsive material.

2332. The device of item 2105, further comprising a visualization agent, wherein the visualization agent comprises a gadolinium chelate.

2333. The device of item 2105, further comprising a visualization agent, wherein the visualization agent comprises iron, magnesium, manganese, copper, or chromium.

2334. The device of item 2105, further comprising a visualization agent, wherein the visualization agent comprises an iron oxide compound.

2335. The device of item 2105, further comprising a visualization agent, wherein the visualization agent comprises a dye, pigment, or colorant.

2336. The device of item 2105, further comprising an echogenic material.

2337. The device of item 2105, further comprising an echogenic material, wherein the echogenic material is in the form of a coating.

2338. The device of item 2105 wherein the device is sterile.

2339. The device of item 2105 wherein the anti-scarring agent is released into tissue in the vicinity of the device after deployment of the device.

2340. The device of item 2105 wherein the anti-scarring agent is released into tissue in the vicinity of the device after deployment of the device, wherein the tissue is connective tissue.

2341. The device of item 2105 wherein the anti-scarring agent is released into tissue in the vicinity of the device after deployment of the device, wherein the tissue is muscle tissue.

2342. The device of item 2105 wherein the anti-scarring agent is released into tissue in the vicinity of the device after deployment of the device, wherein the tissue is nerve tissue.

2343. The device of item 2105 wherein the anti-scarring agent is released into tissue in the vicinity of the device after deployment of the device, wherein the tissue is epithelium tissue.

2344. The device of item 2105 wherein the anti-scarring agent is released in effective concentrations from the device over a period ranging from the time of deployment of the device to about 1 year.

2345. The device of item 2105 wherein the anti-scarring agent is released in effective concentrations from the device over a period ranging from about 1 month to 6 months.

2346. The device of item 2105 wherein the anti-scarring agent is released in effective concentrations from the device over a period ranging from about 1-90 days.

2347. The device of item 2105 wherein the anti-scarring agent is released in effective concentrations from the device at a constant rate.

2348. The device of item 2105 wherein the anti-scarring agent is released in effective concentrations from the device at an increasing rate.

2349. The device of item 2105 wherein the anti-scarring agent is released in effective concentrations from the device at a decreasing rate.

2350. The device of item 2105 wherein the anti-scarring agent is released in effective concentrations from the composition comprising the anti-scarring agent by diffusion over a period ranging from the time of deployment of the device to about 90 days.

2351. The device of item 2105 wherein the anti-scarring agent is released in effective concentrations from the composition comprising the anti-scarring agent by erosion of the composition over a period ranging from the time of deployment of the device to about 90 days.

2352. The device of item 2105 wherein the device comprises about 0.01 μg to about 10 μg of the anti-scarring agent.

2353. The device of item 2105 wherein the device comprises about 10 μg to about 10 mg of the anti-scarring agent.

2354. The device of item 2105 wherein the device comprises about 10 mg to about 250 mg of the anti-scarring agent.

2355. The device of item 2105 wherein the device comprises about 250 mg to about 1000 mg of the anti-scarring agent.

2356. The device of item 2105 wherein the device comprises about 1000 mg to about 2500 mg of the anti-scarring agent.

2357. The device of item 2105 wherein a surface of the device comprises less than 0.01 μg of the anti-scarring agent per mm2 of device surface to which the anti-scarring agent is applied.

2358. The device of item 2105 wherein a surface of the device comprises about 0.01 μg to about 1 μg of the anti-scarring agent per mm2 of device surface to which the anti-scarring agent is applied.

2359. The device of item 2105 wherein a surface of the device comprises about 1 μg to about 10 μg of the anti-scarring agent per mm2 of device surface to which the anti-scarring agent is applied.

2360. The device of item 2105 wherein a surface of the device comprises about 10 μg to about 250 μg of the anti-scarring agent per mm2 of device surface to which the anti-scarring agent is applied.

2361. The device of item 2105 wherein a surface of the device comprises about 250 μg to about 1000 μg of the anti-scarring agent of anti-scarring agent per mm2 of device surface to which the anti-scarring agent is applied.

2362. The device of item 2105 wherein a surface of the device comprises about 1000 μg to about 2500 μg of the anti-scarring agent per mm2 of device surface to which the anti-scarring agent is applied.

2363. The device of any one of items 2105-2362 wherein the implant is a ventriculopleural shunt.

2364. The device of any one of items 2105-2362 wherein the implant is a jugular vein shunt.

2365. The device of any one of items 2105-2362 wherein the implant is a vena cava (VA) shunt.

2366. The device of any one of items 2105-2362 wherein the implant is a ventriculoperitoneal shunt (VP shunt).

2367. The device of any one of items 2105-2362 wherein the implant is a gallbladder shunt.

2368. The device of any one of items 2105-2362 wherein the implant is a peritoneum shunt.

2369. The device of any one of items 2105-2362 wherein the implant is an external ventricular drainage (EVD) device.

2370. The device of any one of items 2105-2362 wherein the implant is an intracranial pressure (ICP) monitoring device.

2371. The device of any one of items 2105-2362 wherein the implant is a dural patch to prevent epidural fibrosis post-laminectomy.

2372. The device of any one of items 2105-2362 wherein the implant is a device for continuous subarachnoid infusions.

2373. A device, comprising an intraocular lens (i.e., an implant) and an anti-scarring agent or a composition comprising an anti-scarring agent, wherein the agent inhibits scarring between the device and a host into which the device is implanted.

2374. The device of item 2373 wherein the agent inhibits cell regeneration.

2375. The device of item 2373 wherein the agent inhibits angiogenesis.

2376. The device of item 2373 wherein the agent inhibits fibroblast migration.

2377. The device of item 2373 wherein the agent inhibits fibroblast proliferation.

2378. The device of item 2373 wherein the agent inhibits deposition of extracellular matrix.

2379. The device of item 2373 wherein the agent inhibits tissue remodeling.

2380. The device of item 2373 wherein the agent is an angiogenesis inhibitor.

2381. The device of item 2373 wherein the agent is a 5-lipoxygenase inhibitor or antagonist.

2382. The device of item 2373 wherein the agent is a chemokine receptor antagonist.

2383. The device of item 2373 wherein the agent is a cell cycle inhibitor.

2384. The device of item 2373 wherein the agent is a taxane.

2385. The device of item 2373 wherein the agent is an anti-microtubule agent.

2386. The device of item 2373 wherein the agent is paclitaxel.

2387. The device of item 2373 wherein the agent is not paclitaxel.

2388. The device of item 2373 wherein the agent is an analogue or derivative of paclitaxel.

2389. The device of item 2373 wherein the agent is a vinca alkaloid.

2390. The device of item 2373 wherein the agent is camptothecin or an analogue or derivative thereof.

2391. The device of item 2373 wherein the agent is a podophyllotoxin.

2392. The device of item 2373 wherein the agent is a podophyllotoxin, wherein the podophyllotoxin is etoposide or an analogue or derivative thereof.

2393. The device of item 2373 wherein the agent is an anthracycline.

2394. The device of item 2373 wherein the agent is an anthracycline, wherein the anthracycline is doxorubicin or an analogue or derivative thereof.

2395. The device of item 2373 wherein the agent is an anthracycline, wherein the anthracycline is mitoxantrone or an analogue or derivative thereof.

2396. The device of item 2373 wherein the agent is a platinum compound.

2397. The device of item 2373 wherein the agent is a nitrosourea.

2398. The device of item 2373 wherein the agent is a nitroimidazole.

2399. The device of item 2373 wherein the agent is a folic acid antagonist.

2400. The device of item 2373 wherein the agent is a cytidine analogue.

2401. The device of item 2373 wherein the agent is a pyrimidine analogue.

2402. The device of item 2373 wherein the agent is a fluoropyrimidine analogue.

2403. The device of item 2373 wherein the agent is a purine analogue.

2404. The device of item 2373 wherein the agent is a nitrogen mustard or an analogue or derivative thereof.

2405. The device of item 2373 wherein the agent is a hydroxyurea.

2406. The device of item 2373 wherein the agent is a mytomicin or an analogue or derivative thereof.

2407. The device of item 2373 wherein the agent is an alkyl sulfonate.

2408. The device of item 2373 wherein the agent is a benzamide or an analogue or derivative thereof.

2409. The device of item 2373 wherein the agent is a nicotinamide or an analogue or derivative thereof.

2410. The device of item 2373 wherein the agent is a halogenated sugar or an analogue or derivative thereof.

2411. The device of item 2373 wherein the agent is a DNA alkylating agent.

2412. The device of item 2373 wherein the agent is an anti-microtubule agent.

2413. The device of item 2373 wherein the agent is a topoisomerase inhibitor.

2414. The device of item 2373 wherein the agent is a DNA cleaving agent.

2415. The device of item 2373 wherein the agent is an antimetabolite.

2416. The device of item 2373 wherein the agent inhibits adenosine deaminase.

2417. The device of item 2373 wherein the agent inhibits purine ring synthesis.

2418. The device of item 2373 wherein the agent is a nucleotide interconversion inhibitor.

2419. The device of item 2373 wherein the agent inhibits dihydrofolate reduction.

2420. The device of item 2373 wherein the agent blocks thymidine monophosphate.

2421. The device of item 2373 wherein the agent causes DNA damage.

2422. The device of item 2373 wherein the agent is a DNA intercalation agent.

2423. The device of item 2373 wherein the agent is a RNA synthesis inhibitor.

2424. The device of item 2373 wherein the agent is a pyrimidine synthesis inhibitor.

2425. The device of item 2373 wherein the agent inhibits ribonucleotide synthesis or function.

2426. The device of item 2373 wherein the agent inhibits thymidine monophosphate synthesis or function.

2427. The device of item 2373 wherein the agent inhibits DNA synthesis.

2428. The device of item 2373 wherein the agent causes DNA adduct formation.

2429. The device of item 2373 wherein the agent inhibits protein synthesis.

2430. The device of item 2373 wherein the agent inhibits microtubule function.

2431. The device of item 2373 wherein the agent is a cyclin dependent protein kinase inhibitor.

2432. The device of item 2373 wherein the agent is an epidermal growth factor kinase inhibitor.

2433. The device of item 2373 wherein the agent is an elastase inhibitor.

2434. The device of item 2373 wherein the agent is a factor Xa inhibitor.

2435. The device of item 2373 wherein the agent is a farnesyltransferase inhibitor.

2436. The device of item 2373 wherein the agent is a fibrinogen antagonist.

2437. The device of item 2373 wherein the agent is a guanylate cyclase stimulant.

2438. The device of item 2373 wherein the agent is a heat shock protein 90 antagonist.

2439. The device of item 2373 wherein the agent is a heat shock protein 90 antagonist, wherein the heat shock protein 90 antagonist is geldanamycin or an analogue or derivative thereof.

2440. The device of item 2373 wherein the agent is a guanylate cyclase stimulant.

2441. The device of item 2373 wherein the agent is a HMGCoA reductase inhibitor.

2442. The device of item 2373 wherein the agent is a HMGCoA reductase inhibitor, wherein the HMGCoA reductase inhibitor is simvastatin or an analogue or derivative thereof.

2443. The device of item 2373 wherein the agent is a hydroorotate dehydrogenase inhibitor.

2444. The device of item 2373 wherein the agent is an IKK2 inhibitor.

2445. The device of item 2373 wherein the agent is an IL-1 antagonist.

2446. The device of item 2373 wherein the agent is an ICE antagonist.

2447. The device of item 2373 wherein the agent is an IRAK antagonist.

2448. The device of item 2373 wherein the agent is an IL-4 agonist.

2449. The device of item 2373 wherein the agent is an immunomodulatory agent.

2450. The device of item 2373 wherein the agent is sirolimus or an analogue or derivative thereof.

2451. The device of item 2373 wherein the agent is not sirolimus.

2452. The device of item 2373 wherein the agent is everolimus or an analogue or derivative thereof.

2453. The device of item 2373 wherein the agent is tacrolimus or an analogue or derivative thereof.

2454. The device of item 2373 wherein the agent is not tacrolimus.

2455. The device of item 2373 wherein the agent is biolmus or an analogue or derivative thereof.

2456. The device of item 2373 wherein the agent is tresperimus or an analogue or derivative thereof.

2457. The device of item 2373 wherein the agent is auranofin or an analogue or derivative thereof.

2458. The device of item 2373 wherein the agent is 27-0-demethylrapamycin or an analogue or derivative thereof.

2459. The device of item 2373 wherein the agent is gusperimus or an analogue or derivative thereof.

2460. The device of item 2373 wherein the agent is pimecrolimus or an analogue or derivative thereof.

2461. The device of item 2373 wherein the agent is ABT-578 or an analogue or derivative thereof.

2462. The device of item 2373 wherein the agent is an inosine monophosphate dehydrogenase (IMPDH) inhibitor.

2463. The device of item 2373 wherein the agent is an IMPDH inhibitor, wherein the IMPDH inhibitor is mycophenolic acid or an analogue or derivative thereof.

2464. The device of item 2373 wherein the agent is an IMPDH inhibitor, wherein the IMPDH inhibitor is 1-alpha-25 dihydroxy vitamin D3 or an analogue or derivative thereof.

2465. The device of item 2373 wherein the agent is a leukotriene inhibitor.

2466. The device of item 2373 wherein the agent is a MCP-1 antagonist.

2467. The device of item 2373 wherein the agent is a MMP inhibitor.

2468. The device of item 2373 wherein the agent is an NF kappa B inhibitor.

2469. The device of item 2373 wherein the agent is an NF kappa B inhibitor, wherein the NF kappa B inhibitor is Bay 11-7082.

2470. The device of item 2373 wherein the agent is an NO agonist.

2471. The device of item 2373 wherein the agent is a p38 MAP kinase inhibitor.

2472. The device of item 2373 wherein the agent is a p38 MAP kinase inhibitor, wherein the p38 MAP kinase inhibitor is SB 202190.

2473. The device of item 2373 wherein the agent is a phosphodiesterase inhibitor.

2474. The device of item 2373 wherein the agent is a TGF beta inhibitor.

2475. The device of item 2373 wherein the agent is a thromboxane A2 antagonist.

2476. The device of item 2373 wherein the agent is a TNFa antagonist.

2477. The device of item 2373 wherein the agent is a TACE inhibitor.

2478. The device of item 2373 wherein the agent is a tyrosine kinase inhibitor.

2479. The device of item 2373 wherein the agent is a vitronectin inhibitor.

2480. The device of item 2373 wherein the agent is a fibroblast growth factor inhibitor.

2481. The device of item 2373 wherein the agent is a protein kinase inhibitor.

2482. The device of item 2373 wherein the agent is a PDGF receptor kinase inhibitor.

2483. The device of item 2373 wherein the agent is an endothelial growth factor receptor kinase inhibitor.

2484. The device of item 2373 wherein the agent is a retinoic acid receptor antagonist.

2485. The device of item 2373 wherein the agent is a platelet derived growth factor receptor kinase inhibitor.

2486. The device of item 2373 wherein the agent is a fibronogin antagonist.

2487. The device of item 2373 wherein the agent is an antimycotic agent.

2488. The device of item 2373 wherein the agent is an antimycotic agent, wherein the antimycotic agent is sulconizole.

2489. The device of item 2373 wherein the agent is a bisphosphonate.

2490. The device of item 2373 wherein the agent is a phospholipase A1 inhibitor.

2491. The device of item 2373 wherein the agent is a histamine H1/H2/H3 receptor antagonist.

2492. The device of item 2373 wherein the agent is a macrolide antibiotic.

2493. The device of item 2373 wherein the agent is a GPIIb/IIIa receptor antagonist.

2494. The device of item 2373 wherein the agent is an endothelin receptor antagonist.

2495. The device of item 2373 wherein the agent is a peroxisome proliferator-activated receptor agonist.

2496. The device of item 2373 wherein the agent is an estrogen receptor agent.

2497. The device of item 2373 wherein the agent is a somastostatin analogue.

2498. The device of item 2373 wherein the agent is a neurokinin 1 antagonist.

2499. The device of item 2373 wherein the agent is a neurokinin 3 antagonist.

2500. The device of item 2373 wherein the agent is a VLA-4 antagonist.

2501. The device of item 2373 wherein the agent is an osteoclast inhibitor.

2502. The device of item 2373 wherein the agent is a DNA topoisomerase ATP hydrolyzing inhibitor.

2503. The device of item 2373 wherein the agent is an angiotensin I converting enzyme inhibitor.

2504. The device of item 2373 wherein the agent is an angiotensin II antagonist.

2505. The device of item 2373 wherein the agent is an enkephalinase inhibitor.

2506. The device of item 2373 wherein the agent is a peroxisome proliferator-activated receptor gamma agonist insulin sensitizer.

2507. The device of item 2373 wherein the agent is a protein kinase C inhibitor.

2508. The device of item 2373 wherein the agent is a ROCK (rho-associated kinase) inhibitor.

2509. The device of item 2373 wherein the agent is a CXCR3 inhibitor.

2510. The device of item 2373 wherein the agent is an Itk inhibitor.

2511. The device of item 2373 wherein the agent is a cytosolic phospholipase A2-alpha inhibitor.

2512. The device of item 2373 wherein the agent is a PPAR agonist.

2513. The device of item 2373 wherein the agent is an immunosuppressant.

2514. The device of item 2373 wherein the agent is an Erb inhibitor.

2515. The device of item 2373 wherein the agent is an apoptosis agonist.

2516. The device of item 2373 wherein the agent is a lipocortin agonist.

2517. The device of item 2373 wherein the agent is a VCAM-1 antagonist.

2518. The device of item 2373 wherein the agent is a collagen antagonist.

2519. The device of item 2373 wherein the agent is an alpha 2 integrin antagonist.

2520. The device of item 2373 wherein the agent is a TNF alpha inhibitor.

2521. The device of item 2373 wherein the agent is a nitric oxide inhibitor 2522. The device of item 2373 wherein the agent is a cathepsin inhibitor.

2523. The device of item 2373 wherein the agent is not an anti-inflammatory agent.

2524. The device of item 2373 wherein the agent is not a steroid.

2525. The device of item 2373 wherein the agent is not a glucocorticosteroid.

2526. The device of item 2373 wherein the agent is not dexamethasone.

2527. The device of item 2373 wherein the agent is not an anti-infective agent.

2528. The device of item 2373 wherein the agent is not an antibiotic.

2529. The device of item 2373 wherein the agent is not an anti-fungal agent.

2530. The device of item 2373, further comprising a polymer.

2531. The device of item 2373, further comprising a polymeric carrier.

2532. The device of item 2373 wherein the anti-scarring agent inhibits adhesion between the device and a host into which the device is implanted.

2533. The device of item 2373 wherein the device delivers the anti-scarring agent locally to tissue proximate to the device.

2534. The device of item 2373, further comprising a coating, wherein the coating comprises the anti-scarring agent.

2535. The device of item 2373, further comprising a coating, wherein the coating is disposed on a surface of the device.

2536. The device of item 2373, further comprising a coating, wherein the coating directly contacts the device.

2537. The device of item 2373, further comprising a coating, wherein the coating indirectly contacts the device.

2538. The device of item 2373, further comprising a coating, wherein the coating partially covers the device.

2539. The device of item 2373, further comprising a coating, wherein the coating completely covers the device.

2540. The device of item 2373, further comprising a coating, wherein the coating is a uniform coating.

2541. The device of item 2373, further comprising a coating, wherein the coating is a non-uniform coating.

2542. The device of item 2373, further comprising a coating, wherein the coating is a discontinuous coating.

2543. The device of item 2373, further comprising a coating, wherein the coating is a patterned coating.

2544. The device of item 2373, further comprising a coating, wherein the coating has a thickness of 100 μm or less.

2545. The device of item 2373, further comprising a coating, wherein the coating has a thickness of 10 μm or less.

2546. The device of item 2373, further comprising a coating, wherein the coating adheres to the surface of the device upon deployment of the device.

2547. The device of item 2373, further comprising a coating, wherein the coating is stable at room temperature for a period of 1 year.

2548. The device of item 2373, further comprising a coating, wherein the anti-scarring agent is present in the coating in an amount ranging between about 0.0001% to about 1% by weight.

2549. The device of item 2373, further comprising a coating, wherein the anti-scarring agent is present in the coating in an amount ranging between about 1% to about 10% by weight.

2550. The device of item 2373, further comprising a coating, wherein the anti-scarring agent is present in the coating in an amount ranging between about 10% to about 25% by weight.

2551. The device of item 2373, further comprising a coating, wherein the anti-scarring agent is present in the coating in an amount ranging between about 25% to about 70% by weight.

2552. The device of item 2373, further comprising a coating, wherein the coating further comprises a polymer.

2553. The device of item 2373, further comprising a first coating having a first composition and the second coating having a second composition.

2554. The device of item 2373, further comprising a first coating having a first composition and the second coating having a second composition, wherein the first composition and the second composition are different.

2555. The device of item 2373, further comprising a polymer.

2556. The device of item 2373, further comprising a polymeric carrier.

2557. The device of item 2373, further comprising a polymeric carrier, wherein the polymeric carrier comprises a copolymer.

2558. The device of item 2373, further comprising a polymeric carrier, wherein the polymeric carrier comprises a block copolymer.

2559. The device of item 2373, further comprising a polymeric carrier, wherein the polymeric carrier comprises a random copolymer.

2560. The device of item 2373, further comprising a polymeric carrier, wherein the polymeric carrier comprises a biodegradable polymer.

2561. The device of item 2373, further comprising a polymeric carrier, wherein the polymeric carrier comprises a non-biodegradable polymer.

2562. The device of item 2373, further comprising a polymeric carrier, wherein the polymeric carrier comprises a hydrophilic polymer.

2563. The device of item 2373, further comprising a polymeric carrier, wherein the polymeric carrier comprises a hydrophobic polymer.

2564. The device of item 2373, further comprising a polymeric carrier, wherein the polymeric carrier comprises a polymer having hydrophilic domains.

2565. The device of item 2373, further comprising a polymeric carrier, wherein the polymeric carrier comprises a polymer having hydrophobic domains.

2566. The device of item 2373, further comprising a polymeric carrier, wherein the polymeric carrier comprises a non-conductive polymer.

2567. The device of item 2373, further comprising a polymeric carrier, wherein the polymeric carrier comprises an elastomer.

2568. The device of item 2373, further comprising a polymeric carrier, wherein the polymeric carrier comprises a hydrogel.

2569. The device of item 2373, further comprising a polymeric carrier, wherein the polymeric carrier comprises a silicone polymer.

2570. The device of item 2373, further comprising a polymeric carrier, wherein the polymeric carrier comprises a hydrocarbon polymer.

2571. The device of item 2373, further comprising a polymeric carrier, wherein the polymeric carrier comprises a styrene-derived polymer.

2572. The device of item 2373, further comprising a polymeric carrier, wherein the polymeric carrier comprises a butadiene polymer.

2573. The device of item 2373, further comprising a polymeric carrier, wherein the polymeric carrier comprises a macromer.

2574. The device of item 2373, further comprising a polymeric carrier, wherein the polymeric carrier comprises a poly(ethylene glycol)polymer.

2575. The device of item 2373, further comprising a polymeric carrier, wherein the polymeric carrier comprises an amorphous polymer.

2576. The device of item 2373, further comprising a lubricious coating.

2577. The device of item 2373 wherein the anti-scarring agent is located within pores or holes of the device.

2578. The device of item 2373 wherein the anti-scarring agent is located within a channel, lumen, or divet of the device.

2579. The device of item 2373, further comprising a second pharmaceutically active agent.

2580. The device of item 2373, further comprising an anti-inflammatory agent.

2581. The device of item 2373, further comprising an agent that inhibits infection.

2582. The device of item 2373, further comprising an agent that inhibits infection, wherein the agent is an anthracycline.

2583. The device of item 2373, further comprising an agent that inhibits infection, wherein the agent is doxorubicin.

2584. The device of item 2373, further comprising an agent that inhibits infection, wherein the agent is mitoxantrone.

2585. The device of item 2373, further comprising an agent that inhibits infection, wherein the agent is a fluoropyrimidine.

2586. The device of item 2373, further comprising an agent that inhibits infection, wherein the agent is 5-fluorouracil (5-FU).

2587. The device of item 2373, further comprising an agent that inhibits infection, wherein the agent is a folic acid antagonist.

2588. The device of item 2373, further comprising an agent that inhibits infection, wherein the agent is methotrexate.

2589. The device of item 2373, further comprising an agent that inhibits infection, wherein the agent is a podophylotoxin.

2590. The device of item 2373, further comprising an agent that inhibits infection, wherein the agent is etoposide.

2591. The device of item 2373, further comprising an agent that inhibits infection, wherein the agent is a camptothecin.

2592. The device of item 2373, further comprising an agent that inhibits infection, wherein the agent is a hydroxyurea.

2593. The device of item 2373, further comprising an agent that inhibits infection, wherein the agent is a platinum complex.

2594. The device of item 2373, further comprising an agent that inhibits infection, wherein the agent is cisplatin.

2595. The device of item 2373, further comprising an anti-thrombotic agent.

2596. The device of item 2373, further comprising a visualization agent.

2597. The device of item 2373, further comprising a visualization agent, wherein the visualization agent is a radiopaque material, wherein the radiopaque material comprises a metal, a halogenated compound, or a barium containing compound.

2598. The device of item 2373, further comprising a visualization agent, wherein the visualization agent is a radiopaque material, wherein the radiopaque material comprises barium, tantalum, or technetium.

2599. The device of item 2373, further comprising a visualization agent, wherein the visualization agent is a MRI responsive material.

2600. The device of item 2373, further comprising a visualization agent, wherein the visualization agent comprises a gadolinium chelate.

2601. The device of item 2373, further comprising a visualization agent, wherein the visualization agent comprises iron, magnesium, manganese, copper, or chromium.

2602. The device of item 2373, further comprising a visualization agent, wherein the visualization agent comprises an iron oxide compound.

2603. The device of item 2373, further comprising a visualization agent, wherein the visualization agent comprises a dye, pigment, or colorant.

2604. The device of item 2373, further comprising an echogenic material.

2605. The device of item 2373, further comprising an echogenic material, wherein the echogenic material is in the form of a coating.

2606. The device of item 2373 wherein the device is sterile.

2607. The device of item 2373 wherein the anti-scarring agent is released into tissue in the vicinity of the device after deployment of the device.

2608. The device of item 2373 wherein the anti-scarring agent is released into tissue in the vicinity of the device after deployment of the device, wherein the tissue is connective tissue.

2609. The device of item 2373 wherein the anti-scarring agent is released into tissue in the vicinity of the device after deployment of the device, wherein the tissue is muscle tissue.

2610. The device of item 2373 wherein the anti-scarring agent is released into tissue in the vicinity of the device after deployment of the device, wherein the tissue is nerve tissue.

2611. The device of item 2373 wherein the anti-scarring agent is released into tissue in the vicinity of the device after deployment of the device, wherein the tissue is epithelium tissue.

2612. The device of item 2373 wherein the anti-scarring agent is released in effective concentrations from the device over a period ranging from the time of deployment of the device to about 1 year.

2613. The device of item 2373 wherein the anti-scarring agent is released in effective concentrations from the device over a period ranging from about 1 month to 6 months.

2614. The device of item 2373 wherein the anti-scarring agent is released in effective concentrations from the device over a period ranging from about 1-90 days.

2615. The device of item 2373 wherein the anti-scarring agent is released in effective concentrations from the device at a constant rate.

2616. The device of item 2373 wherein the anti-scarring agent is released in effective concentrations from the device at an increasing rate.

2617. The device of item 2373 wherein the anti-scarring agent is released in effective concentrations from the device at a decreasing rate.

2618. The device of item 2373 wherein the anti-scarring agent is released in effective concentrations from the composition comprising the anti-scarring agent by diffusion over a period ranging from the time of deployment of the device to about 90 days.

2619. The device of item 2373 wherein the anti-scarring agent is released in effective concentrations from the composition comprising the anti-scarring agent by erosion of the composition over a period ranging from the time of deployment of the device to about 90 days.

2620. The device of item 2373 wherein the device comprises about 0.01 μg to about 10 μg of the anti-scarring agent.

2621. The device of item 2373 wherein the device comprises about 10 μg to about 10 mg of the anti-scarring agent.

2622. The device of item 2373 wherein the device comprises about 10 mg to about 250 mg of the anti-scarring agent.

2623. The device of item 2373 wherein the device comprises about 250 mg to about 1000 mg of the anti-scarring agent.

2624. The device of item 2373 wherein the device comprises about 1000 mg to about 2500 mg of the anti-scarring agent.

2625. The device of item 2373 wherein a surface of the device comprises less than 0.01 μg of the anti-scarring agent per mm2 of device surface to which the anti-scarring agent is applied.

2626. The device of item 2373 wherein a surface of the device comprises about 0.01 μg to about 1 μg of the anti-scarring agent per mm2 of device surface to which the anti-scarring agent is applied.

2627. The device of item 2373 wherein a surface of the device comprises about 1 μg to about 10 μg of the anti-scarring agent per mm2 of device surface to which the anti-scarring agent is applied.

2628. The device of item 2373 wherein a surface of the device comprises about 10 μg to about 250 μg of the anti-scarring agent per mm2 of device surface to which the anti-scarring agent is applied.

2629. The device of item 2373 wherein a surface of the device comprises about 250 μg to about 1000 μg of the anti-scarring agent of anti-scarring agent per mm2 of device surface to which the anti-scarring agent is applied.

2630. The device of item 2373 wherein a surface of the device comprises about 1000 μg to about 2500 μg of the anti-scarring agent per mm2 of device surface to which the anti-scarring agent is applied.

2631. The device of any one of items 2373-2630 wherein the implant is an aphakic lens.

2632. The device of any one of items 2373-2630 wherein the implant is a phakic lens.

2633. The device of any one of items 2373-2630 wherein the implant is a multi-focal lens.

2634. A device, comprising a glaucoma drainage device (i.e., an implant) and an anti-scarring agent or a composition comprising an anti-scarring agent, wherein the agent inhibits scarring between the device and a host into which the device is implanted.

2635. The device of item 2634 wherein the agent inhibits cell regeneration.

2636. The device of item 2634 wherein the agent inhibits angiogenesis.

2637. The device of item 2634 wherein the agent inhibits fibroblast migration.

2638. The device of item 2634 wherein the agent inhibits fibroblast proliferation.

2639. The device of item 2634 wherein the agent inhibits deposition of extracellular matrix.

2640. The device of item 2634 wherein the agent inhibits tissue remodeling.

2641. The device of item 2634 wherein the agent is an angiogenesis inhibitor.

2642. The device of item 2634 wherein the agent is a 5-lipoxygenase inhibitor or antagonist.

2643. The device of item 2634 wherein the agent is a chemokine receptor antagonist.

2644. The device of item 2634 wherein the agent is a cell cycle inhibitor.

2645. The device of item 2634 wherein the agent is a taxane.

2646. The device of item 2634 wherein the agent is an anti-microtubule agent.

2647. The device of item 2634 wherein the agent is paclitaxel.

2648. The device of item 2634 wherein the agent is not paclitaxel.

2649. The device of item 2634 wherein the agent is an analogue or derivative of paclitaxel.

2650. The device of item 2634 wherein the agent is a vinca alkaloid.

2651. The device of item 2634 wherein the agent is camptothecin or an analogue or derivative thereof.

2652. The device of item 2634 wherein the agent is a podophyllotoxin.

2653. The device of item 2634 wherein the agent is a podophyllotoxin, wherein the podophyllotoxin is etoposide or an analogue or derivative thereof.

2654. The device of item 2634 wherein the agent is an anthracycline.

2655. The device of item 2634 wherein the agent is an anthracycline, wherein the anthracycline is doxorubicin or an analogue or derivative thereof.

2656. The device of item 2634 wherein the agent is an anthracycline, wherein the anthracycline is mitoxantrone or an analogue or derivative thereof.

2657. The device of item 2634 wherein the agent is a platinum compound.

2658. The device of item 2634 wherein the agent is a nitrosourea.

2659. The device of item 2634 wherein the agent is a nitroimidazole.

2660. The device of item 2634 wherein the agent is a folic acid antagonist.

2661. The device of item 2634 wherein the agent is a cytidine analogue.

2662. The device of item 2634 wherein the agent is a pyrimidine analogue.

2663. The device of item 2634 wherein the agent is a fluoropyrimidine analogue.

2664. The device of item 2634 wherein the agent is a purine analogue.

2665. The device of item 2634 wherein the agent is a nitrogen mustard or an analogue or derivative thereof.

2666. The device of item 2634 wherein the agent is a hydroxyurea.

2667. The device of item 2634 wherein the agent is a mytomicin or an analogue or derivative thereof.

2668. The device of item 2634 wherein the agent is an alkyl sulfonate.

2669. The device of item 2634 wherein the agent is a benzamide or an analogue or derivative thereof.

2670. The device of item 2634 wherein the agent is a nicotinamide or an analogue or derivative thereof.

2671. The device of item 2634 wherein the agent is a halogenated sugar or an analogue or derivative thereof.

2672. The device of item 2634 wherein the agent is a DNA alkylating agent.

2673. The device of item 2634 wherein the agent is an anti-microtubule agent.

2674. The device of item 2634 wherein the agent is a topoisomerase inhibitor.

2675. The device of item 2634 wherein the agent is a DNA cleaving agent.

2676. The device of item 2634 wherein the agent is an antimetabolite.

2677. The device of item 2634 wherein the agent inhibits adenosine deaminase.

2678. The device of item 2634 wherein the agent inhibits purine ring synthesis.

2679. The device of item 2634 wherein the agent is a nucleotide interconversion inhibitor.

2680. The device of item 2634 wherein the agent inhibits dihydrofolate reduction.

2681. The device of item 2634 wherein the agent blocks thymidine monophosphate.

2682. The device of item 2634 wherein the agent causes DNA damage.

2683. The device of item 2634 wherein the agent is a DNA intercalation agent.

2684. The device of item 2634 wherein the agent is a RNA synthesis inhibitor.

2685. The device of item 2634 wherein the agent is a pyrimidine synthesis inhibitor.

2686. The device of item 2634 wherein the agent inhibits ribonucleotide synthesis or function.

2687. The device of item 2634 wherein the agent inhibits thymidine monophosphate synthesis or function.

2688. The device of item 2634 wherein the agent inhibits DNA synthesis.

2689. The device of item 2634 wherein the agent causes DNA adduct formation.

2690. The device of item 2634 wherein the agent inhibits protein synthesis.

2691. The device of item 2634 wherein the agent inhibits microtubule function.

2692. The device of item 2634 wherein the agent is a cyclin dependent protein kinase inhibitor.

2693. The device of item 2634 wherein the agent is an epidermal growth factor kinase inhibitor.

2694. The device of item 2634 wherein the agent is an elastase inhibitor.

2695. The device of item 2634 wherein the agent is a factor Xa inhibitor.

2696. The device of item 2634 wherein the agent is a farnesyltransferase inhibitor.

2697. The device of item 2634 wherein the agent is a fibrinogen antagonist.

2698. The device of item 2634 wherein the agent is a guanylate cyclase stimulant.

2699. The device of item 2634 wherein the agent is a heat shock protein 90 antagonist.

2700. The device of item 2634 wherein the agent is a heat shock protein 90 antagonist, wherein the heat shock protein 90 antagonist is geldanamycin or an analogue or derivative thereof.

2701. The device of item 2634 wherein the agent is a guanylate cyclase stimulant.

2702. The device of item 2634 wherein the agent is a HMGCoA reductase inhibitor.

2703. The device of item 2634 wherein the agent is a HMGCoA reductase inhibitor, wherein the HMGCoA reductase inhibitor is simvastatin or an analogue or derivative thereof.

2704. The device of item 2634 wherein the agent is a hydroorotate dehydrogenase inhibitor.

2705. The device of item 2634 wherein the agent is an IKK2 inhibitor.

2706. The device of item 2634 wherein the agent is an IL-1 antagonist.

2707. The device of item 2634 wherein the agent is an ICE antagonist.

2708. The device of item 2634 wherein the agent is an IRAK antagonist.

2709. The device of item 2634 wherein the agent is an IL-4 agonist.

2710. The device of item 2634 wherein the agent is an immunomodulatory agent.

2711. The device of item 2634 wherein the agent is sirolimus or an analogue or derivative thereof.

2712. The device of item 2634 wherein the agent is not sirolimus.

2713. The device of item 2634 wherein the agent is everolimus or an analogue or derivative thereof.

2714. The device of item 2634 wherein the agent is tacrolimus or an analogue or derivative thereof.

2715. The device of item 2634 wherein the agent is not tacrolimus.

2716. The device of item 2634 wherein the agent is biolmus or an analogue or derivative thereof.

2717. The device of item 2634 wherein the agent is tresperimus or an analogue or derivative thereof.

2718. The device of item 2634 wherein the agent is auranofin or an analogue or derivative thereof.

2719. The device of item 2634 wherein the agent is 27-0-demethylrapamycin or an analogue or derivative thereof.

2720. The device of item 2634 Wherein the agent is gusperimus or an analogue or derivative thereof.

2721. The device of item 2634 wherein the agent is pimecrolimus or an analogue or derivative thereof.

2722. The device of item 2634 wherein the agent is ABT-578 or an analogue or derivative thereof.

2723. The device of item 2634 wherein the agent is an inosine monophosphate dehydrogenase (IMPDH) inhibitor.

2724. The device of item 2634 wherein the agent is an IMPDH inhibitor, wherein the IMPDH inhibitor is mycophenolic acid or an analogue or derivative thereof.

2725. The device of item 2634 wherein the agent is an IMPDH inhibitor, wherein the IMPDH inhibitor is 1-alpha-25 dihydroxy vitamin D3 or an analogue or derivative thereof.

2726. The device of item 2634 wherein the agent is a leukotriene inhibitor.

2727. The device of item 2634 wherein the agent is a MCP-1 antagonist.

2728. The device of item 2634 wherein the agent is a MMP inhibitor.

2729. The device of item 2634 wherein the agent is an NF kappa B inhibitor.

2730. The device of item 2634 wherein the agent is an NF kappa B inhibitor, wherein the NF kappa B inhibitor is Bay 11-7082.

2731. The device of item 2634 wherein the agent is an NO agonist.

2732. The device of item 2634 wherein the agent is a p38 MAP kinase inhibitor.

2733. The device of item 2634 wherein the agent is a p38 MAP kinase inhibitor, wherein the p38 MAP kinase inhibitor is SB 202190.

2734. The device of item 2634 wherein the agent is a phosphodiesterase inhibitor.

2735. The device of item 2634 wherein the agent is a TGF beta inhibitor.

2736. The device of item 2634 wherein the agent is a thromboxane A2 antagonist.

2737. The device of item 2634 wherein the agent is a TNFa antagonist.

2738. The device of item 2634 wherein the agent is a TACE inhibitor.

2739. The device of item 2634 wherein the agent is a tyrosine kinase inhibitor.

2740. The device of item 2634 wherein the agent is a vitronectin inhibitor.

2741. The device of item 2634 wherein the agent is a fibroblast growth factor inhibitor.

2742. The device of item 2634 wherein the agent is a protein kinase inhibitor.

2743. The device of item 2634 wherein the agent is a PDGF receptor kinase inhibitor.

2744. The device of item 2634 wherein the agent is an endothelial growth factor receptor kinase inhibitor.

2745. The device of item 2634 wherein the agent is a retinoic acid receptor antagonist.

2746. The device of item 2634 wherein the agent is a platelet derived growth factor receptor kinase inhibitor.

2747. The device of item 2634 wherein the agent is a fibronogin antagonist.

2748. The device of item 2634 wherein the agent is an antimycotic agent.

2749. The device of item 2634 wherein the agent is an antimycotic agent, wherein the antimycotic agent is sulconizole.

2750. The device of item 2634 wherein the agent is a bisphosphonate.

2751. The device of item 2634 wherein the agent is a phospholipase A1 inhibitor.

2752. The device of item 2634 wherein the agent is a histamine H1/H2/H3 receptor antagonist.

2753. The device of item 2634 wherein the agent is a macrolide antibiotic.

2754. The device of item 2634 wherein the agent is a GPIIb/IIIa receptor antagonist.

2755. The device of item 2634 wherein the agent is an endothelin receptor antagonist.

2756. The device of item 2634 wherein the agent is a peroxisome proliferator-activated receptor agonist.

2757. The device of item 2634 wherein the agent is an estrogen receptor agent.

2758. The device of item 2634 wherein the agent is a somastostatin analogue.

2759. The device of item 2634 wherein the agent is a neurokinin 1 antagonist.

2760. The device of item 2634 wherein the agent is a neurokinin 3 antagonist.

2761. The device of item 2634 wherein the agent is a VLA-4 antagonist.

2762. The device of item 2634 wherein the agent is an osteoclast inhibitor.

2763. The device of item 2634 wherein the agent is a DNA topoisomerase ATP hydrolyzing inhibitor.

2764. The device of item 2634 wherein the agent is an angiotensin I converting enzyme inhibitor.

2765. The device of item 2634 wherein the agent is an angiotensin II antagonist.

2766. The device of item 2634 wherein the agent is an enkephalinase inhibitor.

2767. The device of item 2634 wherein the agent is a peroxisome proliferator-activated receptor gamma agonist insulin sensitizer.

2768. The device of item 2634 wherein the agent is a protein kinase C inhibitor.

2769. The device of item 2634 wherein the agent is a ROCK (rho-associated kinase) inhibitor.

2770. The device of item 2634 wherein the agent is a CXCR3 inhibitor.

2771. The device of item 2634 wherein the agent is an Itk inhibitor.

2772. The device of item 2634 wherein the agent is a cytosolic phospholipase A2-alpha inhibitor.

2773. The device of item 2634 wherein the agent is a PPAR agonist.

2774. The device of item 2634 wherein the agent is an immunosuppressant.

2775. The device of item 2634 wherein the agent is an Erb inhibitor.

2776. The device of item 2634 wherein the agent is an apoptosis agonist.

2777. The device of item 2634 wherein the agent is a lipocortin agonist.

2778. The device of item 2634 wherein the agent is a VCAM-1 antagonist.

2779. The device of item 2634 wherein the agent is a collagen antagonist.

2780. The device of item 2634 wherein the agent is an alpha 2 integrin antagonist.

2781. The device of item 2634 wherein the agent is a TNF alpha inhibitor.

2782. The device of item 2634 wherein the agent is a nitric oxide inhibitor.

2783. The device of item 2634 wherein the agent is a cathepsin inhibitor.

2784. The device of item 2634 wherein the agent is not an anti-inflammatory agent.

2785. The device of item 2634 wherein the agent is not a steroid.

2786. The device of item 2634 wherein the agent is not a glucocorticosteroid.

2787. The device of item 2634 wherein the agent is not dexamethasone.

2788. The device of item 2634 wherein the agent is not an anti-infective agent.

2789. The device of item 2634 wherein the agent is not an antibiotic.

2790. The device of item 2634 wherein the agent is not an anti-fungal agent.

2791. The device of item 2634, further comprising a polymer.

2792. The device of item 2634, further comprising a polymeric carrier.

2793. The device of item 2634 wherein the anti-scarring agent inhibits adhesion between the device and a host into which the device is implanted.

2794. The device of item 2634 wherein the device delivers the anti-scarring agent locally to tissue proximate to the device.

2795. The device of item 2634, further comprising a coating, wherein the coating comprises the anti-scarring agent.

2796. The device of item 2634, further comprising a coating, wherein the coating is disposed on a surface of the device.

2797. The device of item 2634, further comprising a coating, wherein the coating directly contacts the device.

2798. The device of item 2634, further comprising a coating, wherein the coating indirectly contacts the device.

2799. The device of item 2634, further comprising a coating, wherein the coating partially covers the device.

2800. The device of item 2634, further comprising a coating, wherein the coating completely covers the device.

2801. The device of item 2634, further comprising a coating, wherein the coating is a uniform coating.

2802. The device of item 2634, further comprising a coating, wherein the coating is a non-uniform coating.

2803. The device of item 2634, further comprising a coating, wherein the coating is a discontinuous coating.

2804. The device of item 2634, further comprising a coating, wherein the coating is a patterned coating.

2805. The device of item 2634, further comprising a coating, wherein the coating has a thickness of 100 μm or less.

2806. The device of item 2634, further comprising a coating, wherein the coating has a thickness of 10 μm or less.

2807. The device of item 2634, further comprising a coating, wherein the coating adheres to the surface of the device upon deployment of the device.

2808. The device of item 2634, further comprising a coating, wherein the coating is stable at room temperature for a period of 1 year.

2809. The device of item 2634, further comprising a coating, wherein the anti-scarring agent is present in the coating in an amount ranging between about 0.0001% to about 1% by weight.

2810. The device of item 2634, further comprising a coating, wherein the anti-scarring agent is present in the coating in an amount ranging between about 1% to about 10% by weight.

2811. The device of item 2634, further comprising a coating, wherein the anti-scarring agent is present in the coating in an amount ranging between about 10% to about 25% by weight.

2812. The device of item 2634, further comprising a coating, wherein the anti-scarring agent is present in the coating in an amount ranging between about 25% to about 70% by weight.

2813. The device of item 2634, further comprising a coating, wherein the coating further comprises a polymer.

2814. The device of item 2634, further comprising a first coating having a first composition and the second coating having a second composition.

2815. The device of item 2634, further comprising a first coating having a first composition and the second coating having a second composition, wherein the first composition and the second composition are different.

2816. The device of item 2634, further comprising a polymer.

2817. The device of item 2634, further comprising a polymeric carrier.

2818. The device of item 2634, further comprising a polymeric carrier, wherein the polymeric carrier comprises a copolymer.

2819. The device of item 2634, further comprising a polymeric carrier, wherein the polymeric carrier comprises a block copolymer.

2820. The device of item 2634, further comprising a polymeric carrier, wherein the polymeric carrier comprises a random copolymer.

2821. The device of item 2634, further comprising a polymeric carrier, wherein the polymeric carrier comprises a biodegradable polymer.

2822. The device of item 2634, further comprising a polymeric carrier, wherein the polymeric carrier comprises a non-biodegradable polymer.

2823. The device of item 2634, further comprising a polymeric carrier, wherein the polymeric carrier comprises a hydrophilic polymer.

2824. The device of item 2634, further comprising a polymeric carrier, wherein the polymeric carrier comprises a hydrophobic polymer.

2825. The device of item 2634, further comprising a polymeric carrier, wherein the polymeric carrier comprises a polymer having hydrophilic domains.

2826. The device of item 2634, further comprising a polymeric carrier, wherein the polymeric carrier comprises a polymer having hydrophobic domains.

2827. The device of item 2634, further comprising a polymeric carrier, wherein the polymeric carrier comprises a non-conductive polymer.

2828. The device of item 2634, further comprising a polymeric carrier, wherein the polymeric carrier comprises an elastomer.

2829. The device of item 2634, further comprising a polymeric carrier, wherein the polymeric carrier comprises a hydrogel.

2830. The device of item 2634, further comprising a polymeric carrier, wherein the polymeric carrier comprises a silicone polymer.

2831. The device of item 2634, further comprising a polymeric carrier, wherein the polymeric carrier comprises a hydrocarbon polymer.

2832. The device of item 2634, further comprising a polymeric carrier, wherein the polymeric carrier comprises a styrene-derived polymer.

2833. The device of item 2634, further comprising a polymeric carrier, wherein the polymeric carrier comprises a butadiene polymer.

2834. The device of item 2634, further comprising a polymeric carrier, wherein the polymeric carrier comprises a macromer.

2835. The device of item 2634, further comprising a polymeric carrier, wherein the polymeric carrier comprises a poly(ethylene glycol)polymer.

2836. The device of item 2634, further comprising a polymeric carrier, wherein the polymeric carrier comprises an amorphous polymer.

2837. The device of item 2634, further comprising a lubricious coating.

2838. The device of item 2634 wherein the anti-scarring agent is located within pores or holes of the device.

2839. The device of item 2634 wherein the anti-scarring agent is located within a channel, lumen, or divet of the device.

2840. The device of item 2634, further comprising a second pharmaceutically active agent.

2841. The device of item 2634, further comprising an anti-inflammatory agent.

2842. The device of item 2634, further comprising an agent that inhibits infection.

2843. The device of item 2634, further comprising an agent that inhibits infection, wherein the agent is an anthracycline.

2844. The device of item 2634, further comprising an agent that inhibits infection, wherein the agent is doxorubicin.

2845. The device of item 2634, further comprising an agent that inhibits infection, wherein the agent is mitoxantrone.

2846. The device of item 2634, further comprising an agent that inhibits infection, wherein the agent is a fluoropyrimidine.

2847. The device of item 2634, further comprising an agent that inhibits infection, wherein the agent is 5-fluorouracil (5-FU).

2848. The device of item 2634, further comprising an agent that inhibits infection, wherein the agent is a folic acid antagonist.

2849. The device of item 2634, further comprising an agent that inhibits infection, wherein the agent is methotrexate.

2850. The device of item 2634, further comprising an agent that inhibits infection, wherein the agent is a podophylotoxin.

2851. The device of item 2634, further comprising an agent that inhibits infection, wherein the agent is etoposide.

2852. The device of item 2634, further comprising an agent that inhibits infection, wherein the agent is a camptothecin.

2853. The device of item 2634, further comprising an agent that inhibits infection, wherein the agent is a hydroxyurea.

2854. The device of item 2634, further comprising an agent that inhibits infection, wherein the agent is a platinum complex.

2855. The device of item 2634, further comprising an agent that inhibits infection, wherein the agent is cisplatin.

2856. The device of item 2634, further comprising an anti-thrombotic agent.

2857. The device of item 2634, further comprising a visualization agent.

2858. The device of item 2634, further comprising a visualization agent, wherein the visualization agent is a radiopaque material, wherein the radiopaque material comprises a metal, a halogenated compound, or a barium containing compound.

2859. The device of item 2634, further comprising a visualization agent, wherein the visualization agent is a radiopaque material, wherein the radiopaque material comprises barium, tantalum, or technetium.

2860. The device of item 2634, further comprising a visualization agent, wherein the visualization agent is a MRI responsive material.

2861. The device of item 2634, further comprising a visualization agent, wherein the visualization agent comprises a gadolinium chelate.

2862. The device of item 2634, further comprising a visualization agent, wherein the visualization agent comprises iron, magnesium, manganese, copper, or chromium.

2863. The device of item 2634, further comprising a visualization agent, wherein the visualization agent comprises an iron oxide compound.

2864. The device of item 2634, further comprising a visualization agent, wherein the visualization agent comprises a dye, pigment, or colorant.

2865. The device of item 2634, further comprising an echogenic material.

2866. The device of item 2634, further comprising an echogenic material, wherein the echogenic material is in the form of a coating.

2867. The device of item 2634 wherein the device is sterile.

2868. The device of item 2634 wherein the anti-scarring agent is released into tissue in the vicinity of the device after deployment of the device.

2869. The device of item 2634 wherein the anti-scarring agent is released into tissue in the vicinity of the device after deployment of the device, wherein the tissue is connective tissue.

2870. The device of item 2634 wherein the anti-scarring agent is released into tissue in the vicinity of the device after deployment of the device, wherein the tissue is muscle tissue.

2871. The device of item 2634 wherein the anti-scarring agent is released into tissue in the vicinity of the device after deployment of the device, wherein the tissue is nerve tissue.

2872. The device of item 2634 wherein the anti-scarring agent is released into tissue in the vicinity of the device after deployment of the device, wherein the tissue is epithelium tissue.

2873. The device of item 2634 wherein the anti-scarring agent is released in effective concentrations from the device over a period ranging from the time of deployment of the device to about 1 year.

2874. The device of item 2634 wherein the anti-scarring agent is released in effective concentrations from the device over a period ranging from about 1 month to 6 months.

2875. The device of item 2634 wherein the anti-scarring agent is released in effective concentrations from the device over a period ranging from about 1-90 days.

2876. The device of item 2634 wherein the anti-scarring agent is released in effective concentrations from the device at a constant rate.

2877. The device of item 2634 wherein the anti-scarring agent is released in effective concentrations from the device at an increasing rate.

2878. The device of item 2634 wherein the anti-scarring agent is released in effective concentrations from the device at a decreasing rate.

2879. The device of item 2634 wherein the anti-scarring agent is released in effective concentrations from the composition comprising the anti-scarring agent by diffusion over a period ranging from the time of deployment of the device to about 90 days.

2880. The device of item 2634 wherein the anti-scarring agent is released in effective concentrations from the composition comprising the anti-scarring agent by erosion of the composition over a period ranging from the time of deployment of the device to about 90 days.

2881. The device of item 2634 wherein the device comprises about 0.01 μg to about 10 μg of the anti-scarring agent.

2882. The device of item 2634 wherein the device comprises about 10 μg to about 10 mg of the anti-scarring agent.

2883. The device of item 2634 wherein the device comprises about 10 mg to about 250 mg of the anti-scarring agent.

2884. The device of item 2634 wherein the device comprises about 250 mg to about 1000 mg of the anti-scarring agent.

2885. The device of item 2634 wherein the device comprises about 1000 mg to about 2500 mg of the anti-scarring agent.

2886. The device of item 2634 wherein a surface of the device comprises less than 0.01 μg of the anti-scarring agent per mm2 of device surface to which the anti-scarring agent is applied.

2887. The device of item 2634 wherein a surface of the device comprises about 0.01 μg to about 1 μg of the anti-scarring agent per mm2 of device surface to which the anti-scarring agent is applied.

2888. The device of item 2634 wherein a surface of the device comprises about 1 μg to about 10 μg of the anti-scarring agent per mm2 of device surface to which the anti-scarring agent is applied.

2889. The device of item 2634 wherein a surface of the device comprises about 10 μg to about 250 μg of the anti-scarring agent per mm2 of device surface to which the anti-scarring agent is applied.

2890. The device of item 2634 wherein a surface of the device comprises about 250 μg to about 1000 μg of the anti-scarring agent of anti-scarring agent per mm2 of device surface to which the anti-scarring agent is applied.

2891. The device of item 2634 wherein a surface of the device comprises about 1000 μg to about 2500 μg of the anti-scarring agent per mm2 of device surface to which the anti-scarring agent is applied.

2892. The device of any one of items 2634-2891 wherein the implant is an episcleral drainage plate or tube.

2893. A device, comprising a penile implant and an anti-scarring agent or a composition comprising an anti-scarring agent, wherein the agent inhibits scarring between the device and a host into which the device is implanted.

2894. The device of item 2893 wherein the agent inhibits cell regeneration.

2895. The device of item 2893 wherein the agent inhibits angiogenesis.

2896. The device of item 2893 wherein the agent inhibits fibroblast migration.

2897. The device of item 2893 wherein the agent inhibits fibroblast proliferation.

2898. The device of item 2893 wherein the agent inhibits deposition of extracellular matrix.

2899. The device of item 2893 wherein the agent inhibits tissue remodeling.

2900. The device of item 2893 wherein the agent is an angiogenesis inhibitor.

2901. The device of item 2893 wherein the agent is a 5-lipoxygenase inhibitor or antagonist.

2902. The device of item 2893 wherein the agent is a chemokine receptor antagonist.

2903. The device of item 2893 wherein the agent is a cell cycle inhibitor.

2904. The device of item 2893 wherein the agent is a taxane.

2905. The device of item 2893 wherein the agent is an anti-microtubule agent.

2906. The device of item 2893 wherein the agent is paclitaxel.

2907. The device of item 2893 wherein the agent is not paclitaxel.

2908. The device of item 2893 wherein the agent is an analogue or derivative of paclitaxel.

2909. The device of item 2893 wherein the agent is a vinca alkaloid.

2910. The device of item 2893 wherein the agent is camptothecin or an analogue or derivative thereof.

2911. The device of item 2893 wherein the agent is a podophyllotoxin.

2912. The device of item 2893 wherein the agent is a podophyllotoxin, wherein the podophyllotoxin is etoposide or an analogue or derivative thereof.

2913. The device of item 2893 wherein the agent is an anthracycline.

2914. The device of item 2893 wherein the agent is an anthracycline, wherein the anthracycline is doxorubicin or an analogue or derivative thereof.

2915. The device of item 2893 wherein the agent is an anthracycline, wherein the anthracycline is mitoxantrone or an analogue or derivative thereof.

2916. The device of item 2893 wherein the agent is a platinum compound.

2917. The device of item 2893 wherein the agent is a nitrosourea.

2918. The device of item 2893 wherein the agent is a nitroimidazole.

2919. The device of item 2893 wherein the agent is a folic acid antagonist.

2920. The device of item 2893 wherein the agent is a cytidine analogue.

2921. The device of item 2893 wherein the agent is a pyrimidine analogue.

2922. The device of item 2893 wherein the agent is a fluoropyrimidine analogue.

2923. The device of item 2893 wherein the agent is a purine analogue.

2924. The device of item 2893 wherein the agent is a nitrogen mustard or an analogue or derivative thereof.

2925. The device of item 2893 wherein the agent is a hydroxyurea.

2926. The device of item 2893 wherein the agent is a mytomicin or an analogue or derivative thereof.

2927. The device of item 2893 wherein the agent is an alkyl sulfonate.

2928. The device of item 2893 wherein the agent is a benzamide or an analogue or derivative thereof.

2929. The device of item 2893 wherein the agent is a nicotinamide or an analogue or derivative thereof.

2930. The device of item 2893 wherein the agent is a halogenated sugar or an analogue or derivative thereof.

2931. The device of item 2893 wherein the agent is a DNA alkylating agent.

2932. The device of item 2893 wherein the agent is an anti-microtubule agent.

2933. The device of item 2893 wherein the agent is a topoisomerase inhibitor.

2934. The device of item 2893 wherein the agent is a DNA cleaving agent.

2935. The device of item 2893 wherein the agent is an antimetabolite.

2936. The device of item 2893 wherein the agent inhibits adenosine deaminase.

2937. The device of item 2893 wherein the agent inhibits purine ring synthesis.

2938. The device of item 2893 wherein the agent is a nucleotide interconversion inhibitor.

2939. The device of item 2893 wherein the agent inhibits dihydrofolate reduction.

2940. The device of item 2893 wherein the agent blocks thymidine monophosphate.

2941. The device of item 2893 wherein the agent causes DNA damage.

2942. The device of item 2893 wherein the agent is a DNA intercalation agent.

2943. The device of item 2893 wherein the agent is a RNA synthesis inhibitor.

2944. The device of item 2893 wherein the agent is a pyrimidine synthesis inhibitor.

2945. The device of item 2893 wherein the agent inhibits ribonucleotide synthesis or function.

2946. The device of item 2893 wherein the agent inhibits thymidine monophosphate synthesis or function.

2947. The device of item 2893 wherein the agent inhibits DNA synthesis.

2948. The device of item 2893 wherein the agent causes DNA adduct formation.

2949. The device of item 2893 wherein the agent inhibits protein synthesis.

2950. The device of item 2893 wherein the agent inhibits microtubule function.

2951. The device of item 2893 wherein the agent is a cyclin dependent protein kinase inhibitor.

2952. The device of item 2893 wherein the agent is an epidermal growth factor kinase inhibitor.

2953. The device of item 2893 wherein the agent is an elastase inhibitor.

2954. The device of item 2893 wherein the agent is a factor Xa inhibitor.

2955. The device of item 2893 wherein the agent is a farnesyltransferase inhibitor.

2956. The device of item 2893 wherein the agent is a fibrinogen antagonist.

2957. The device of item 2893 wherein the agent is a guanylate cyclase stimulant.

2958. The device of item 2893 wherein the agent is a heat shock protein 90 antagonist.

2959. The device of item 2893 wherein the agent is a heat shock protein 90 antagonist, wherein the heat shock protein 90 antagonist is geldanamycin or an analogue or derivative thereof.

2960. The device of item 2893 wherein the agent is a guanylate cyclase stimulant.

2961. The device of item 2893 wherein the agent is a HMGCoA reductase inhibitor.

2962. The device of item 2893 wherein the agent is a HMGCoA reductase inhibitor, wherein the HMGCoA reductase inhibitor is simvastatin or an analogue or derivative thereof.

2963. The device of item 2893 wherein the agent is a hydroorotate dehydrogenase inhibitor.

2964. The device of item 2893 wherein the agent is an IKK2 inhibitor.

2965. The device of item 2893 wherein the agent is an IL-1 antagonist.

2966. The device of item 2893 wherein the agent is an ICE antagonist.

2967. The device of item 2893 wherein the agent is an IRAK antagonist.

2968. The device of item 2893 wherein the agent is an IL-4 agonist.

2969. The device of item 2893 wherein the agent is an immunomodulatory agent.

2970. The device of item 2893 wherein the agent is sirolimus or an analogue or derivative thereof.

2971. The device of item 2893 wherein the agent is not sirolimus.

2972. The device of item 2893 wherein the agent is everolimus or an analogue or derivative thereof.

2973. The device of item 2893 wherein the agent is tacrolimus or an analogue or derivative thereof.

2974. The device of item 2893 wherein the agent is not tacrolimus.

2975. The device of item 2893 wherein the agent is biolmus or an analogue or derivative thereof.

2976. The device of item 2893 wherein the agent is tresperimus or an analogue or derivative thereof.

2977. The device of item 2893 wherein the agent is auranofin or an analogue or derivative thereof.

2978. The device of item 2893 wherein the agent is 27-0-demethylrapamycin or an analogue or derivative thereof.

2979. The device of item 2893 wherein the agent is gusperimus or an analogue or derivative thereof.

2980. The device of item 2893 wherein the agent is pimecrolimus or an analogue or derivative thereof.

2981. The device of item 2893 wherein the agent is ABT-578 or an analogue or derivative thereof.

2982. The device of item 2893 wherein the agent is an inosine monophosphate dehydrogenase (IMPDH) inhibitor.

2983. The device of item 2893 wherein the agent is an IMPDH inhibitor, wherein the IMPDH inhibitor is mycophenolic acid or an analogue or derivative thereof.

2984. The device of item 2893 wherein the agent is an IMPDH inhibitor, wherein the IMPDH inhibitor is 1-alpha-25 dihydroxy vitamin D3 or an analogue or derivative thereof.

2985. The device of item 2893 wherein the agent is a leukotriene inhibitor.

2986. The device of item 2893 wherein the agent is a MCP-1 antagonist.

2987. The device of item 2893 wherein the agent is a MMP inhibitor.

2988. The device of item 2893 wherein the agent is an NF kappa B inhibitor.

2989. The device of item 2893 wherein the agent is an NF kappa B inhibitor, wherein the NF kappa B inhibitor is Bay 11-7082.

2990. The device of item 2893 wherein the agent is an NO agonist.

2991. The device of item 2893 wherein the agent is a p38 MAP kinase inhibitor.

2992. The device of item 2893 wherein the agent is a p38 MAP kinase inhibitor, wherein the p38 MAP kinase inhibitor is SB 202190.

2993. The device of item 2893 wherein the agent is a phosphodiesterase inhibitor.

2994. The device of item 2893 wherein the agent is a TGF beta inhibitor.

2995. The device of item 2893 wherein the agent is a thromboxane A2 antagonist.

2996. The device of item 2893 wherein the agent is a TNFa antagonist.

2997. The device of item 2893 wherein the agent is a TACE inhibitor.

2998. The device of item 2893 wherein the agent is a tyrosine kinase inhibitor.

2999. The device of item 2893 wherein the agent is a vitronectin inhibitor.

3000. The device of item 2893 wherein the agent is a fibroblast growth factor inhibitor.

3001. The device of item 2893 wherein the agent is a protein kinase inhibitor.

3002. The device of item 2893 wherein the agent is a PDGF receptor kinase inhibitor.

3003. The device of item 2893 wherein the agent is an endothelial growth factor receptor kinase inhibitor.

3004. The device of item 2893 wherein the agent is a retinoic acid receptor antagonist.

3005. The device of item 2893 wherein the agent is a platelet derived growth factor receptor kinase inhibitor.

3006. The device of item 2893 wherein the agent is a fibronogin antagonist.

3007. The device of item 2893 wherein the agent is an antimycotic agent.

3008. The device of item 2893 wherein the agent is an antimycotic agent, wherein the antimycotic agent is sulconizole.

3009. The device of item 2893 wherein the agent is a bisphosphonate.

3010. The device of item 2893 wherein the agent is a phospholipase A1 inhibitor.

3011. The device of item 2893 wherein the agent is a histamine H1/H2/H3 receptor antagonist.

3012. The device of item 2893 wherein the agent is a macrolide antibiotic.

3013. The device of item 2893 wherein the agent is a GPIIb/IIIa receptor antagonist.

3014. The device of item 2893 wherein the agent is an endothelin receptor antagonist.

3015. The device of item 2893 wherein the agent is a peroxisome proliferator-activated receptor agonist.

3016. The device of item 2893 wherein the agent is an estrogen receptor agent.

3017. The device of item 2893 wherein the agent is a somastostatin analogue.

3018. The device of item 2893 wherein the agent is a neurokinin 1 antagonist.

3019. The device of item 2893 wherein the agent is a neurokinin 3 antagonist.

3020. The device of item 2893 wherein the agent is a VLA-4 antagonist.

3021. The device of item 2893 wherein the agent is an osteoclast inhibitor.

3022. The device of item 2893 wherein the agent is a DNA topoisomerase ATP hydrolyzing inhibitor.

3023. The device of item 2893 wherein the agent is an angiotensin I converting enzyme inhibitor.

3024. The device of item 2893 wherein the agent is an angiotensin II antagonist.

3025. The device of item 2893 wherein the agent is an enkephalinase inhibitor.

3026. The device of item 2893 wherein the agent is a peroxisome proliferator-activated receptor gamma agonist insulin sensitizer.

3027. The device of item 2893 wherein the agent is a protein kinase C inhibitor.

3028. The device of item 2893 wherein the agent is a ROCK (rho-associated kinase) inhibitor.

3029. The device of item 2893 wherein the agent is a CXCR3 inhibitor.

3030. The device of item 2893 wherein the agent is an Itk inhibitor.

3031. The device of item 2893 wherein the agent is a cytosolic phospholipase A2-alpha inhibitor.

3032. The device of item 2893 wherein the agent is a PPAR agonist.

3033. The device of item 2893 wherein the agent is an immunosuppressant.

3034. The device of item 2893 wherein the agent is an Erb inhibitor.

3035. The device of item 2893 wherein the agent is an apoptosis agonist.

3036. The device of item 2893 wherein the agent is a lipocortin agonist.

3037. The device of item 2893 wherein the agent is a VCAM-1 antagonist.

3038. The device of item 2893 wherein the agent is a collagen antagonist.

3039. The device of item 2893 wherein the agent is an alpha 2 integrin antagonist.

3040. The device of item 2893 wherein the agent is a TNF alpha inhibitor.

3041. The device of item 2893 wherein the agent is a nitric oxide inhibitor.

3042. The device of item 2893 wherein the agent is a cathepsin inhibitor.

3043. The device of item 2893 wherein the agent is not an anti-inflammatory agent.

3044. The device of item 2893 wherein the agent is not a steroid.

3045. The device of item 2893 wherein the agent is not a glucocorticosteroid.

3046. The device of item 2893 wherein the agent is not dexamethasone.

3047. The device of item 2893 wherein the agent is not an anti-infective agent.

3048. The device of item 2893 wherein the agent is not an antibiotic.

3049. The device of item 2893 wherein the agent is not an anti-fungal agent.

3050. The device of item 2893, further comprising a polymer.

3051. The device of item 2893, further comprising a polymeric carrier.

3052. The device of item 2893 wherein the anti-scarring agent inhibits adhesion between the device and a host into which the device is implanted.

3053. The device of item 2893 wherein the device delivers the anti-scarring agent locally to tissue proximate to the device.

3054. The device of item 2893, further comprising a coating, wherein the coating comprises the anti-scarring agent.

3055. The device of item 2893, further comprising a coating, wherein the coating is disposed on a surface of the device.

3056. The device of item 2893, further comprising a coating, wherein the coating directly contacts the device.

3057. The device of item 2893, further comprising a coating, wherein the coating indirectly contacts the device.

3058. The device of item 2893, further comprising a coating, wherein the coating partially covers the device.

3059. The device of item 2893, further comprising a coating, wherein the coating completely covers the device.

3060. The device of item 2893, further comprising a coating, wherein the coating is a uniform coating.

3061. The device of item 2893, further comprising a coating, wherein the coating is a non-uniform coating.

3062. The device of item 2893, further comprising a coating, wherein the coating is a discontinuous coating.

3063. The device of item 2893, further comprising a coating, wherein the coating is a patterned coating.

3064. The device of item 2893, further comprising a coating, wherein the coating has a thickness of 100 μm or less.

3065. The device of item 2893, further comprising a coating, wherein the coating has a thickness of 10 μm or less.

3066. The device of item 2893, further comprising a coating, wherein the coating adheres to the surface of the device upon deployment of the device.

3067. The device of item 2893, further comprising a coating, wherein the coating is stable at room temperature for a period of 1 year.

3068. The device of item 2893, further comprising a coating, wherein the anti-scarring agent is present in the coating in an amount ranging between about 0.0001% to about 1% by weight.

3069. The device of item 2893, further comprising a coating, wherein the anti-scarring agent is present in the coating in an amount ranging between about 1% to about 10% by weight.

3070. The device of item 2893, further comprising a coating, wherein the anti-scarring agent is present in the coating in an amount ranging between about 10% to about 25% by weight.

3071. The device of item 2893, further comprising a coating, wherein the anti-scarring agent is present in the coating in an amount ranging between about 25% to about 70% by weight.

3072. The device of item 2893, further comprising a coating, wherein the coating further comprises a polymer.

3073. The device of item 2893, further comprising a first coating having a first composition and the second coating having a second composition.

3074. The device of item 2893, further comprising a first coating having a first composition and the second coating having a second composition, wherein the first composition and the second composition are different.

3075. The device of item 2893, further comprising a polymer.

3076. The device of item 2893, further comprising a polymeric carrier.

3077. The device of item 2893, further comprising a polymeric carrier, wherein the polymeric carrier comprises a copolymer.

3078. The device of item 2893, further comprising a polymeric carrier, wherein the polymeric carrier comprises a block copolymer.

3079. The device of item 2893, further comprising a polymeric carrier, wherein the polymeric carrier comprises a random copolymer.

3080. The device of item 2893, further comprising a polymeric carrier, wherein the polymeric carrier comprises a biodegradable polymer.

3081. The device of item 2893, further comprising a polymeric carrier, wherein the polymeric carrier comprises a non-biodegradable polymer.

3082. The device of item 2893, further comprising a polymeric carrier, wherein the polymeric carrier comprises a hydrophilic polymer.

3083. The device of item 2893, further comprising a polymeric carrier, wherein the polymeric carrier comprises a hydrophobic polymer.

3084. The device of item 2893, further comprising a polymeric carrier, wherein the polymeric carrier comprises a polymer having hydrophilic domains.

3085. The device of item 2893, further comprising a polymeric carrier, wherein the polymeric carrier comprises a polymer having hydrophobic domains.

3086. The device of item 2893, further comprising a polymeric carrier, wherein the polymeric carrier comprises a non-conductive polymer.

3087. The device of item 2893, further comprising a polymeric carrier, wherein the polymeric carrier comprises an elastomer.

3088. The device of item 2893, further comprising a polymeric carrier, wherein the polymeric carrier comprises a hydrogel.

3089. The device of item 2893, further comprising a polymeric carrier, wherein the polymeric carrier comprises a silicone polymer.

3090. The device of item 2893, further comprising a polymeric carrier, wherein the polymeric carrier comprises a hydrocarbon polymer.

3091. The device of item 2893, further comprising a polymeric carrier, wherein the polymeric carrier comprises a styrene-derived polymer.

3092. The device of item 2893, further comprising a polymeric carrier, wherein the polymeric carrier comprises a butadiene polymer.

3093. The device of item 2893, further comprising a polymeric carrier, wherein the polymeric carrier comprises a macromer.

3094. The device of item 2893, further comprising a polymeric carrier, wherein the polymeric carrier comprises a poly(ethylene glycol)polymer.

3095. The device of item 2893, further comprising a polymeric carrier, wherein the polymeric carrier comprises an amorphous polymer.

3096. The device of item 2893, further comprising a lubricious coating.

3097. The device of item 2893 wherein the anti-scarring agent is located within pores or holes of the device.

3098. The device of item 2893 wherein the anti-scarring agent is located within a channel, lumen, or divet of the device.

3099. The device of item 2893, further comprising a second pharmaceutically active agent.

3100. The device of item 2893, further comprising an anti-inflammatory agent.

3101. The device of item 2893, further comprising an agent that inhibits infection.

3102. The device of item 2893, further comprising an agent that inhibits infection, wherein the agent is an anthracycline.

3103. The device of item 2893, further comprising an agent that inhibits infection, wherein the agent is doxorubicin.

3104. The device of item 2893, further comprising an agent that inhibits infection, wherein the agent is mitoxantrone.

3105. The device of item 2893, further comprising an agent that inhibits infection, wherein the agent is a fluoropyrimidine.

3106. The device of item 2893, further comprising an agent that inhibits infection, wherein the agent is 5-fluorouracil (5-FU).

3107. The device of item 2893, further comprising an agent that inhibits infection, wherein the agent is a folic acid antagonist.

3108. The device of item 2893, further comprising an agent that inhibits infection, wherein the agent is methotrexate.

3109. The device of item 2893, further comprising an agent that inhibits infection, wherein the agent is a podophylotoxin.

3110. The device of item 2893, further comprising an agent that inhibits infection, wherein the agent is etoposide.

3111. The device of item 2893, further comprising an agent that inhibits infection, wherein the agent is a camptothecin.

3112. The device of item 2893, further comprising an agent that inhibits infection, wherein the agent is a hydroxyurea.

3113. The device of item 2893, further comprising an agent that inhibits infection, wherein the agent is a platinum complex.

3114. The device of item 2893, further comprising an agent that inhibits infection, wherein the agent is cisplatin.

3115. The device of item 2893, further comprising an anti-thrombotic agent.

3116. The device of item 2893, further comprising a visualization agent.

3117. The device of item 2893, further comprising a visualization agent, wherein the visualization agent is a radiopaque material, wherein the radiopaque material comprises a metal, a halogenated compound, or a barium containing compound.

3118. The device of item 2893, further comprising a visualization agent, wherein the visualization agent is a radiopaque material, wherein the radiopaque material comprises barium, tantalum, or technetium.

3119. The device of item 2893, further comprising a visualization agent, wherein the visualization agent is a MRI responsive material.

3120. The device of item 2893, further comprising a visualization agent, wherein the visualization agent comprises a gadolinium chelate.

3121. The device of item 2893, further comprising a visualization agent, wherein the visualization agent comprises iron, magnesium, manganese, copper, or chromium.

3122. The device of item 2893, further comprising a visualization agent, wherein the visualization agent comprises an iron oxide compound.

3123. The device of item 2893, further comprising a visualization agent, wherein the visualization agent comprises a dye, pigment, or colorant.

3124. The device of item 2893, further comprising an echogenic material.

3125. The device of item 2893, further comprising an echogenic material, wherein the echogenic material is in the form of a coating.

3126. The device of item 2893 wherein the device is sterile.

3127. The device of item 2893 wherein the anti-scarring agent is released into tissue in the vicinity of the device after deployment of the device.

3128. The device of item 2893 wherein the anti-scarring agent is released into tissue in the vicinity of the device after deployment of the device, wherein the tissue is connective tissue.

3129. The device of item 2893 wherein the anti-scarring agent is released into tissue in the vicinity of the device after deployment of the device, wherein the tissue is muscle tissue.

3130. The device of item 2893 wherein the anti-scarring agent is released into tissue in the vicinity of the device after deployment of the device, wherein the tissue is nerve tissue.

3131. The device of item 2893 wherein the anti-scarring agent is released into tissue in the vicinity of the device after deployment of the device, wherein the tissue is epithelium tissue.

3132. The device of item 2893 wherein the anti-scarring agent is released in effective concentrations from the device over a period ranging from the time of deployment of the device to about 1 year.

3133. The device of item 2893 wherein the anti-scarring agent is released in effective concentrations from the device over a period ranging from about 1 month to 6 months.

3134. The device of item 2893 wherein the anti-scarring agent is released in effective concentrations from the device over a period ranging from about 1-90 days.

3135. The device of item 2893 wherein the anti-scarring agent is released in effective concentrations from the device at a constant rate.

3136. The device of item 2893 wherein the anti-scarring agent is released in effective concentrations from the device at an increasing rate.

3137. The device of item 2893 wherein the anti-scarring agent is released in effective concentrations from the device at a decreasing rate.

3138. The device of item 2893 wherein the anti-scarring agent is released in effective concentrations from the composition comprising the anti-scarring agent by diffusion over a period ranging from the time of deployment of the device to about 90 days.

3139. The device of item 2893 wherein the anti-scarring agent is released in effective concentrations from the composition comprising the anti-scarring agent by erosion of the composition over a period ranging from the time of deployment of the device to about 90 days.

3140. The device of item 2893 wherein the device comprises about 0.01 μg to about 10 μg of the anti-scarring agent.

3141. The device of item 2893 wherein the device comprises about 10 μg to about 10 mg of the anti-scarring agent.

3142. The device of item 2893 wherein the device comprises about 10 mg to about 250 mg of the anti-scarring agent.

3143. The device of item 2893 wherein the device comprises about 250 mg to about 1000 mg of the anti-scarring agent.

3144. The device of item 2893 wherein the device comprises about 1000 mg to about 2500 mg of the anti-scarring agent.

3145. The device of item 2893 wherein a surface of the device comprises less than 0.01 μg of the anti-scarring agent per mm2 of device surface to which the anti-scarring agent is applied.

3146. The device of item 2893 wherein a surface of the device comprises about 0.01 μg to about 1 μg of the anti-scarring agent per mm2 of device surface to which the anti-scarring agent is applied.

3147. The device of item 2893 wherein a surface of the device comprises about 1 μg to about 10 μg of the anti-scarring agent per mm2 of device surface to which the anti-scarring agent is applied.

3148. The device of item 2893 wherein a surface of the device comprises about 10 μg to about 250 μg of the anti-scarring agent per mm2 of device surface to which the anti-scarring agent is applied.

3149. The device of item 2893 wherein a surface of the device comprises about 250 μg to about 1000 μg of the anti-scarring agent of anti-scarring agent per mm2 of device surface to which the anti-scarring agent is applied.

3150. The device of item 2893 wherein a surface of the device comprises about 1000 μg to about 2500 μg of the anti-scarring agent per mm2 of device surface to which the anti-scarring agent is applied.

3151. The device of any one of items 2893-3150 wherein the implant is a flexible rod or coil.

3152. The device of any one of items 2893-3150 wherein the implant comprises an inflatable tube and a pump.

3153. The device of any one of items 2893-3150 wherein the implant comprises a pressure chamber.

3154. A device, comprising an endotracheal tube (i.e., an implant) and an anti-scarring agent or a composition comprising an anti-scarring agent, wherein the agent inhibits scarring between the device and a host into which the device is implanted.

3155. The device of item 3154 wherein the agent inhibits cell regeneration.

3156. The device of item 3154 wherein the agent inhibits angiogenesis.

3157. The device of item 3154 wherein the agent inhibits fibroblast migration.

3158. The device of item 3154 wherein the agent inhibits fibroblast proliferation.

3159. The device of item 3154 wherein the agent inhibits deposition of extracellular matrix.

3160. The device of item 3154 wherein the agent inhibits tissue remodeling.

3161. The device of item 3154 wherein the agent is an angiogenesis inhibitor.

3162. The device of item 3154 wherein the agent is a 5-lipoxygenase inhibitor or antagonist.

3163. The device of item 3154 wherein the agent is a chemokine receptor antagonist.

3164. The device of item 3154 wherein the agent is a cell cycle inhibitor.

3165. The device of item 3154 wherein the agent is a taxane.

3166. The device of item 3154 wherein the agent is an anti-microtubule agent.

3167. The device of item 3154 wherein the agent is paclitaxel.

3168. The device of item 3154 wherein the agent is not paclitaxel.

3169. The device of item 3154 wherein the agent is an analogue or derivative of paclitaxel.

3170. The device of item 3154 wherein the agent is a vinca alkaloid.

3171. The device of item 3154 wherein the agent is camptothecin or an analogue or derivative thereof.

3172. The device of item 3154 wherein the agent is a podophyllotoxin.

3173. The device of item 3154 wherein the agent is a podophyllotoxin, wherein the podophyllotoxin is etoposide or an analogue or derivative thereof.

3174. The device of item 3154 wherein the agent is an anthracycline.

3175. The device of item 3154 wherein the agent is an anthracycline, wherein the anthracycline is doxorubicin or an analogue or derivative thereof.

3176. The device of item 3154 wherein the agent is an anthracycline, wherein the anthracycline is mitoxantrone or an analogue or derivative thereof.

3177. The device of item 3154 wherein the agent is a platinum compound.

3178. The device of item 3154 wherein the agent is a nitrosourea.

3179. The device of item 3154 wherein the agent is a nitroimidazole.

3180. The device of item 3154 wherein the agent is a folic acid antagonist.

3181. The device of item 3154 wherein the agent is a cytidine analogue.

3182. The device of item 3154 wherein the agent is a pyrimidine analogue.

3183. The device of item 3154 wherein the agent is a fluoropyrimidine analogue.

3184. The device of item 3154 wherein the agent is a purine analogue.

3185. The device of item 3154 wherein the agent is a nitrogen mustard or an analogue or derivative thereof.

3186. The device of item 3154 wherein the agent is a hydroxyurea.

3187. The device of item 3154 wherein the agent is a mytomicin or an analogue or derivative thereof.

3188. The device of item 3154 wherein the agent is an alkyl sulfonate.

3189. The device of item 3154 wherein the agent is a benzamide or an analogue or derivative thereof.

3190. The device of item 3154 wherein the agent is a nicotinamide or an analogue or derivative thereof.

3191. The device of item 3154 wherein the agent is a halogenated sugar or an analogue or derivative thereof.

3192. The device of item 3154 wherein the agent is a DNA alkylating agent.

3193. The device of item 3154 wherein the agent is an anti-microtubule agent.

3194. The device of item 3154 wherein the agent is a topoisomerase inhibitor.

3195. The device of item 3154 wherein the agent is a DNA cleaving agent.

3196. The device of item 3154 wherein the agent is an antimetabolite.

3197. The device of item 3154 wherein the agent inhibits adenosine deaminase.

3198. The device of item 3154 wherein the agent inhibits purine ring synthesis.

3199. The device of item 3154 wherein the agent is a nucleotide interconversion inhibitor.

3200. The device of item 3154 wherein the agent inhibits dihydrofolate reduction.

3201. The device of item 3154 wherein the agent blocks thymidine mono phosphate.

3202. The device of item 3154 wherein the agent causes DNA damage.

3203. The device of item 3154 wherein the agent is a DNA intercalation agent.

3204. The device of item 3154 wherein the agent is a RNA synthesis inhibitor.

3205. The device of item 3154 wherein the agent is a pyrimidine synthesis inhibitor.

3206. The device of item 3154 wherein the agent inhibits ribonucleotide synthesis or function.

3207. The device of item 3154 wherein the agent inhibits thymidine monophosphate synthesis or function.

3208. The device of item 3154 wherein the agent inhibits DNA synthesis.

3209. The device of item 3154 wherein the agent causes DNA adduct formation.

3210. The device of item 3154 wherein the agent inhibits protein synthesis.

3211. The device of item 3154 wherein the agent inhibits microtubule function.

3212. The device of item 3154 wherein the agent is a cyclin dependent protein kinase inhibitor.

3213. The device of item 3154 wherein the agent is an epidermal growth factor kinase inhibitor.

3214. The device of item 3154 wherein the agent is an elastase inhibitor.

3215. The device of item 3154 wherein the agent is a factor Xa inhibitor.

3216. The device of item 3154 wherein the agent is a farnesyltransferase inhibitor.

3217. The device of item 3154 wherein the agent is a fibrinogen antagonist.

3218. The device of item 3154 wherein the agent is a guanylate cyclase stimulant.

3219. The device of item 3154 wherein the agent is a heat shock protein 90 antagonist.

3220. The device of item 3154 wherein the agent is a heat shock protein 90 antagonist, wherein the heat shock protein 90 antagonist is geldanamycin or an analogue or derivative thereof.

3221. The device of item 3154 wherein the agent is a guanylate cyclase stimulant.

3222. The device of item 3154 wherein the agent is a HMGCoA reductase inhibitor.

3223. The device of item 3154 wherein the agent is a HMGCoA reductase inhibitor, wherein the HMGCoA reductase inhibitor is simvastatin or an analogue or derivative thereof.

3224. The device of item 3154 wherein the agent is a hydroorotate dehydrogenase inhibitor.

3225. The device of item 3154 wherein the agent is an IKK2 inhibitor.

3226. The device of item 3154 wherein the agent is an IL-1 antagonist.

3227. The device of item 3154 wherein the agent is an ICE antagonist.

3228. The device of item 3154 wherein the agent is an IRAK antagonist.

3229. The device of item 3154 wherein the agent is an IL-4 agonist.

3230. The device of item 3154 wherein the agent is an immunomodulatory agent.

3231. The device of item 3154 wherein the agent is sirolimus or an analogue or derivative thereof.

3232. The device of item 3154 wherein the agent is not sirolimus.

3233. The device of item 3154 wherein the agent is everolimus or an analogue or derivative thereof.

3234. The device of item 3154 wherein the agent is tacrolimus or an analogue or derivative thereof.

3235. The device of item 3154 wherein the agent is not tacrolimus.

3236. The device of item 3154 wherein the agent is biolmus or an analogue or derivative thereof.

3237. The device of item 3154 wherein the agent is tresperimus or an analogue or derivative thereof.

3238. The device of item 3154 wherein the agent is auranofin or an analogue or derivative thereof.

3239. The device of item 3154 wherein the agent is 27-0-demethylrapamycin or an analogue or derivative thereof.

3240. The device of item 3154 wherein the agent is gusperimus or an analogue or derivative thereof.

3241. The device of item 3154 wherein the agent is pimecrolimus or an analogue or derivative thereof.

3242. The device of item 3154 wherein the agent is ABT-578 or an analogue or derivative thereof.

3243. The device of item 3154 wherein the agent is an inosine monophosphate dehydrogenase (IMPDH) inhibitor.

3244. The device of item 3154 wherein the agent is an IMPDH inhibitor, wherein the IMPDH inhibitor is mycophenolic acid or an analogue or derivative thereof.

3245. The device of item 3154 wherein the agent is an IMPDH inhibitor, wherein the IMPDH inhibitor is 1-alpha-25 dihydroxy vitamin D3 or an analogue or derivative thereof.

3246. The device of item 3154 wherein the agent is a leukotriene inhibitor.

3247. The device of item 3154 wherein the agent is a MCP-1 antagonist.

3248. The device of item 3154 wherein the agent is a MMP inhibitor.

3249. The device of item 3154 wherein the agent is an NF kappa B inhibitor.

3250. The device of item 3154 wherein the agent is an NF kappa B inhibitor, wherein the NF kappa B inhibitor is Bay 11-7082.

3251. The device of item 3154 wherein the agent is an NO agonist.

3252. The device of item 3154 wherein the agent is a p38 MAP kinase inhibitor.

3253. The device of item 3154 wherein the agent is a p38 MAP kinase inhibitor, wherein the p38 MAP kinase inhibitor is SB 202190.

3254. The device of item 3154 wherein the agent is a phosphodiesterase inhibitor.

3255. The device of item 3154 wherein the agent is a TGF beta inhibitor.

3256. The device of item 3154 wherein the agent is a thromboxane A2 antagonist.

3257. The device of item 3154 wherein the agent is a TNFa antagonist.

3258. The device of item 3154 wherein the agent is a TACE inhibitor.

3259. The device of item 3154 wherein the agent is a tyrosine kinase inhibitor.

3260. The device of item 3154 wherein the agent is a vitronectin inhibitor.

3261. The device of item 3154 wherein the agent is a fibroblast growth factor inhibitor.

3262. The device of item 3154 wherein the agent is a protein kinase inhibitor.

3263. The device of item 3154 wherein the agent is a PDGF receptor kinase inhibitor.

3264. The device of item 3154 wherein the agent is an endothelial growth factor receptor kinase inhibitor.

3265. The device of item 3154 wherein the agent is a retinoic acid receptor antagonist.

3266. The device of item 3154 wherein the agent is a platelet derived growth factor receptor kinase inhibitor.

3267. The device of item 3154 wherein the agent is a fibronogin antagonist.

3268. The device of item 3154 wherein the agent is an antimycotic agent.

3269. The device of item 3154 wherein the agent is an antimycotic agent, wherein the antimycotic agent is sulconizole.

3270. The device of item 3154 wherein the agent is a bisphosphonate.

3271. The device of item 3154 wherein the agent is a phospholipase A1 inhibitor.

3272. The device of item 3154 wherein the agent is a histamine H1/H2/H3 receptor antagonist.

3273. The device of item 3154 wherein the agent is a macrolide antibiotic.

3274. The device of item 3154 wherein the agent is a GPIIb/IIIa receptor antagonist.

3275. The device of item 3154 wherein the agent is an endothelin receptor antagonist.

3276. The device of item 3154 wherein the agent is a peroxisome proliferator-activated receptor agonist.

3277. The device of item 3154 wherein the agent is an estrogen receptor agent.

3278. The device of item 3154 wherein the agent is a somastostatin analogue.

3279. The device of item 3154 wherein the agent is a neurokinin 1 antagonist.

3280. The device of item 3154 wherein the agent is a neurokinin 3 antagonist.

3281. The device of item 3154 wherein the agent is a VLA-4 antagonist.

3282. The device of item 3154 wherein the agent is an osteoclast inhibitor.

3283. The device of item 3154 wherein the agent is a DNA topoisomerase ATP hydrolyzing inhibitor.

3284. The device of item 3154 wherein the agent is an angiotensin I converting enzyme inhibitor.

3285. The device of item 3154 wherein the agent is an angiotensin II antagonist.

3286. The device of item 3154 wherein the agent is an enkephalinase inhibitor.

3287. The device of item 3154 wherein the agent is a peroxisome proliferator-activated receptor gamma agonist insulin sensitizer.

3288. The device of item 3154 wherein the agent is a protein kinase C inhibitor.

3289. The device of item 3154 wherein the agent is a ROCK (rho-associated kinase) inhibitor.

3290. The device of item 3154 wherein the agent is a CXCR3 inhibitor.

3291. The device of item 3154 wherein the agent is an Itk inhibitor.

3292. The device of item 3154 wherein the agent is a cytosolic phospholipase A2-alpha inhibitor.

3293. The device of item 3154 wherein the agent is a PPAR agonist.

3294. The device of item 3154 wherein the agent is an immunosuppressant.

3295. The device of item 3154 wherein the agent is an Erb inhibitor.

3296. The device of item 3154 wherein the agent is an apoptosis agonist.

3297. The device of item 3154 wherein the agent is a lipocortin agonist.

3298. The device of item 3154 wherein the agent is a VCAM-1 antagonist.

3299. The device of item 3154 wherein the agent is a collagen antagonist.

3300. The device of item 3154 wherein the agent is an alpha 2 integrin antagonist.

3301. The device of item 3154 wherein the agent is a TNF alpha inhibitor.

3302. The device of item 3154 wherein the agent is a nitric oxide inhibitor.

3303. The device of item 3154 wherein the agent is a cathepsin inhibitor.

3304. The device of item 3154 wherein the agent is not an anti-inflammatory agent.

3305. The device of item 3154 wherein the agent is not a steroid.

3306. The device of item 3154 wherein the agent is not a glucocorticosteroid.

3307. The device of item 3154 wherein the agent is not dexamethasone.

3308. The device of item 3154 wherein the agent is not an anti-infective agent.

3309. The device of item 3154 wherein the agent is not an antibiotic.

3310. The device of item 3154 wherein the agent is not an anti-fungal agent.

3311. The device of item 3154, further comprising a polymer.

3312. The device of item 3154, further comprising a polymeric carrier.

3313. The device of item 3154 wherein the anti-scarring agent inhibits adhesion between the device and a host into which the device is implanted.

3314. The device of item 3154 wherein the device delivers the anti-scarring agent locally to tissue proximate to the device.

3315. The device of item 3154, further comprising a coating, wherein the coating comprises the anti-scarring agent.

3316. The device of item 3154, further comprising a coating, wherein the coating is disposed on a surface of the device.

3317. The device of item 3154, further comprising a coating, wherein the coating directly contacts the device.

3318. The device of item 3154, further comprising a coating, wherein the coating indirectly contacts the device.

3319. The device of item 3154, further comprising a coating, wherein the coating partially covers the device.

3320. The device of item 3154, further comprising a coating, wherein the coating completely covers the device.

3321. The device of item 3154, further comprising a coating, wherein the coating is a uniform coating.

3322. The device of item 3154, further comprising a coating, wherein the coating is a non-uniform coating.

3323. The device of item 3154, further comprising a coating, wherein the coating is a discontinuous coating.

3324. The device of item 3154, further comprising a coating, wherein the coating is a patterned coating.

3325. The device of item 3154, further comprising a coating, wherein the coating has a thickness of 100 μm or less.

3326. The device of item 3154, further comprising a coating, wherein the coating has a thickness of 10 μm or less.

3327. The device of item 3154, further comprising a coating, wherein the coating adheres to the surface of the device upon deployment of the device.

3328. The device of item 3154, further comprising a coating, wherein the coating is stable at room temperature for a period of 1 year.

3329. The device of item 3154, further comprising a coating, wherein the anti-scarring agent is present in the coating in an amount ranging between about 0.0001% to about 1% by weight.

3330. The device of item 3154, further comprising a coating, wherein the anti-scarring agent is present in the coating in an amount ranging between about 1% to about 10% by weight.

3331. The device of item 3154, further comprising a coating, wherein the anti-scarring agent is present in the coating in an amount ranging between about 10% to about 25% by weight.

3332. The device of item 3154, further comprising a coating, wherein the anti-scarring agent is present in the coating in an amount ranging between about 25% to about 70% by weight.

3333. The device of item 3154, further comprising a coating, wherein the coating further comprises a polymer.

3334. The device of item 3154, further comprising a first coating having a first composition and the second coating having a second composition.

3335. The device of item 3154, further comprising a first coating having a first composition and the second coating having a second composition, wherein the first composition and the second composition are different.

3336. The device of item 3154, further comprising a polymer.

3337. The device of item 3154, further comprising a polymeric carrier.

3338. The device of item 3154, further comprising a polymeric carrier, wherein the polymeric carrier comprises a copolymer.

3339. The device of item 3154, further comprising a polymeric carrier, wherein the polymeric carrier comprises a block copolymer.

3340. The device of item 3154, further comprising a polymeric carrier, wherein the polymeric carrier comprises a random copolymer.

3341. The device of item 3154, further comprising a polymeric carrier, wherein the polymeric carrier comprises a biodegradable polymer.

3342. The device of item 31.54, further comprising a polymeric carrier, wherein the polymeric carrier comprises a non-biodegradable polymer.

3343. The device of item 3154, further comprising a polymeric carrier, wherein the polymeric carrier comprises a hydrophilic polymer.

3344. The device of item 3154, further comprising a polymeric carrier, wherein the polymeric carrier comprises a hydrophobic polymer.

3345. The device of item 3154, further comprising a polymeric carrier, wherein the polymeric carrier comprises a polymer having hydrophilic domains.

3346. The device of item 3154, further comprising a polymeric carrier, wherein the polymeric carrier comprises a polymer having hydrophobic domains.

3347. The device of item 3154, further comprising a polymeric carrier, wherein the polymeric carrier comprises a non-conductive polymer.

3348. The device of item 3154, further comprising a polymeric carrier, wherein the polymeric carrier comprises an elastomer.

3349. The device of item 3154, further comprising a polymeric carrier, wherein the polymeric carrier comprises a hydrogel.

3350. The device of item 3154, further comprising a polymeric carrier, wherein the polymeric carrier comprises a silicone polymer.

3351. The device of item 3154, further comprising a polymeric carrier, wherein the polymeric carrier comprises a hydrocarbon polymer.

3352. The device of item 3154, further comprising a polymeric carrier, wherein the polymeric carrier comprises a styrene-derived polymer.

3353. The device of item 3154, further comprising a polymeric carrier, wherein the polymeric carrier comprises a butadiene polymer.

3354. The device of item 3154, further comprising a polymeric carrier, wherein the polymeric carrier comprises a macromer.

3355. The device of item 3154, further comprising a polymeric carrier, wherein the polymeric carrier comprises a poly(ethylene glycol)polymer.

3356. The device of item 3154, further comprising a polymeric carrier, wherein the polymeric carrier comprises an amorphous polymer.

3357. The device of item 3154, further comprising a lubricious coating.

3358. The device of item 3154 wherein the anti-scarring agent is located within pores or holes of the device.

3359. The device of item 3154 wherein the anti-scarring agent is located within a channel, lumen, or divet of the device.

3360. The device of item 3154, further comprising a second pharmaceutically active agent.

3361. The device of item 3154, further comprising an anti-inflammatory agent.

3362. The device of item 3154, further comprising an agent that inhibits infection.

3363. The device of item 3154, further comprising an agent that inhibits infection, wherein the agent is an anthracycline.

3364. The device of item 3154, further comprising an agent that inhibits infection, wherein the agent is doxorubicin.

3365. The device of item 3154, further comprising an agent that inhibits infection, wherein the agent is mitoxantrone.

3366. The device of item 3154, further comprising an agent that inhibits infection, wherein the agent is a fluoropyrimidine.

3367. The device of item 3154, further comprising an agent that inhibits infection, wherein the agent is 5-fluorouracil (5-FU).

3368. The device of item 3154, further comprising an agent that inhibits infection, wherein the agent is a folic acid antagonist.

3369. The device of item 3154, further comprising an agent that inhibits infection, wherein the agent is methotrexate.

3370. The device of item 3154, further comprising an agent that inhibits infection, wherein the agent is a podophylotoxin.

3371. The device of item 3154, further comprising an agent that inhibits infection, wherein the agent is etoposide.

3372. The device of item 3154, further comprising an agent that inhibits infection, wherein the agent is a camptothecin.

3373. The device of item 3154, further comprising an agent that inhibits infection, wherein the agent is a hydroxyurea.

3374. The device of item 3154, further comprising an agent that inhibits infection, wherein the agent is a platinum complex.

3375. The device of item 3154, further comprising an agent that inhibits infection, wherein the agent is cisplatin.

3376. The device of item 3154, further comprising an anti-thrombotic agent.

3377. The device of item 3154, further comprising a visualization agent.

3378. The device of item 3154, further comprising a visualization agent, wherein the visualization agent is a radiopaque material, wherein the radiopaque material comprises a metal, a halogenated compound, or a barium containing compound.

3379. The device of item 3154, further comprising a visualization agent, wherein the visualization agent is a radiopaque material, wherein the radiopaque material comprises barium, tantalum, or technetium.

3380. The device of item 3154, further comprising a visualization agent, wherein the visualization agent is a MRI responsive material.

3381. The device of item 3154, further comprising a visualization agent, wherein the visualization agent comprises a gadolinium chelate.

3382. The device of item 3154, further comprising a visualization agent, wherein the visualization agent comprises iron, magnesium, manganese, copper, or chromium.

3383. The device of item 3154, further comprising a visualization agent, wherein the visualization agent comprises an iron oxide compound.

3384. The device of item 3154, further comprising a visualization agent, wherein the visualization agent comprises a dye, pigment, or colorant.

3385. The device of item 3154, further comprising an echogenic material.

3386. The device of item 3154, further comprising an echogenic material, wherein the echogenic material is in the form of a coating.

3387. The device of item 3154 wherein the device is sterile.

3388. The device of item 3154 wherein the anti-scarring agent is released into tissue in the vicinity of the device after deployment of the device.

3389. The device of item 3154 wherein the anti-scarring agent is released into tissue in the vicinity of the device after deployment of the device, wherein the tissue is connective tissue.

3390. The device of item 3154 wherein the anti-scarring agent is released into tissue in the vicinity of the device after deployment of the device, wherein the tissue is muscle tissue.

3391. The device of item 3154 wherein the anti-scarring agent is released into tissue in the vicinity of the device after deployment of the device, wherein the tissue is nerve tissue.

3392. The device of item 3154 wherein the anti-scarring agent is released into tissue in the vicinity of the device after deployment of the device, wherein the tissue is epithelium tissue.

3393. The device of item 3154 wherein the anti-scarring agent is released in effective concentrations from the device over a period ranging from the time of deployment of the device to about 1 year.

3394. The device of item 3154 wherein the anti-scarring agent is released in effective concentrations from the device over a period ranging from about 1 month to 6 months.

3395. The device of item 3154 wherein the anti-scarring agent is released in effective concentrations from the device over a period ranging from about 1-90 days.

3396. The device of item 3154 wherein the anti-scarring agent is released in effective concentrations from the device at a constant rate.

3397. The device of item 3154 wherein the anti-scarring agent is released in effective concentrations from the device at an increasing rate.

3398. The device of item 3154 wherein the anti-scarring agent is released in effective concentrations from the device at a decreasing rate.

3399. The device of item 3154 wherein the anti-scarring agent is released in effective concentrations from the composition comprising the anti-scarring agent by diffusion over a period ranging from the time of deployment of the device to about 90 days.

3400. The device of item 3154 wherein the anti-scarring agent is released in effective concentrations from the composition comprising the anti-scarring agent by erosion of the composition over a period ranging from the time of deployment of the device to about 90 days.

3401. The device of item 3154 wherein the device comprises about 0.01 μg to about 10 μg of the anti-scarring agent.

3402. The device of item 3154 wherein the device comprises about 10 μg to about 10 mg of the anti-scarring agent.

3403. The device of item 3154 wherein the device comprises about 10 mg to about 250 mg of the anti-scarring agent.

3404. The device of item 3154 wherein the device comprises about 250 mg to about 1000 mg of the anti-scarring agent.

3405. The device of item 3154 wherein the device comprises about 1000 mg to about 2500 mg of the anti-scarring agent.

3406. The device of item 3154 wherein a surface of the device comprises less than 0.01 μg of the anti-scarring agent per mm2 of device surface to which the anti-scarring agent is applied.

3407. The device of item 3154 wherein a surface of the device comprises about 0.01 μg to about 1 μg of the anti-scarring agent per mm2 of device surface to which the anti-scarring agent is applied.

3408. The device of item 3154 wherein a surface of the device comprises about 1 μg to about 10 μg of the anti-scarring agent per mm2 of device surface to which the anti-scarring agent is applied.

3409. The device of item 3154 wherein a surface of the device comprises about 10 μg to about 250 μg of the anti-scarring agent per mm2 of device surface to which the anti-scarring agent is applied.

3410. The device of item 3154 wherein a surface of the device comprises about 250 μg to about 1000 μg of the anti-scarring agent of anti-scarring agent per mm2 of device surface to which the anti-scarring agent is applied.

3411. The device of item 3154 wherein a surface of the device comprises about 1000 μg to about 2500 μg of the anti-scarring agent per mm2 of device surface to which the anti-scarring agent is applied.

3412. A device, comprising a tracheostomy tube (i.e., an implant) and an anti-scarring agent or a composition comprising an anti-scarring agent, wherein the agent inhibits scarring between the device and a host into which the device is implanted.

3413. The device of item 3412 wherein the agent inhibits cell regeneration.

3414. The device of item 3412 wherein the agent inhibits angiogenesis.

3415. The device of item 3412 wherein the agent inhibits fibroblast migration.

3416. The device of item 3412 wherein the agent inhibits fibroblast proliferation.

3417. The device of item 3412 wherein the agent inhibits deposition of extracellular matrix.

3418. The device of item 3412 wherein the agent inhibits tissue remodeling.

3419. The device of item 3412 wherein the agent is an angiogenesis inhibitor.

3420. The device of item 3412 wherein the agent is a 5-lipoxygenase inhibitor or antagonist.

3421. The device of item 3412 wherein the agent is a chemokine receptor antagonist.

3422. The device of item 3412 wherein the agent is a cell cycle inhibitor.

3423. The device of item 3412 wherein the agent is a taxane.

3424. The device of item 3412 wherein the agent is an anti-microtubule agent.

3425. The device of item 3412 wherein the agent is paclitaxel.

3426. The device of item 3412 wherein the agent is not paclitaxel.

3427. The device of item 3412 wherein the agent is an analogue or derivative of paclitaxel.

3428. The device of item 3412 wherein the agent is a vinca alkaloid.

3429. The device of item 3412 wherein the agent is camptothecin or an analogue or derivative thereof.

3430. The device of item 3412 wherein the agent is a podophyllotoxin.

3431. The device of item 3412 wherein the agent is a podophyllotoxin, wherein the podophyllotoxin is etoposide or an analogue or derivative thereof.

3432. The device of item 3412 wherein the agent is an anthracycline.

3433. The device of item 3412 wherein the agent is an anthracycline, wherein the anthracycline is doxorubicin or an analogue or derivative thereof.

3434. The device of item 3412 wherein the agent is an anthracycline, wherein the anthracycline is mitoxantrone or an analogue or derivative thereof.

3435. The device of item 3412 wherein the agent is a platinum compound.

3436. The device of item 3412 wherein the agent is a nitrosourea.

3437. The device of item 3412 wherein the agent is a nitroimidazole.

3438. The device of item 3412 wherein the agent is a folic acid antagonist.

3439. The device of item 3412 wherein the agent is a cytidine analogue.

3440. The device of item 3412 wherein the agent is a pyrimidine analogue.

3441. The device of item 3412 wherein the agent is a fluoropyrimidine analogue.

3442. The device of item 3412 wherein the agent is a purine analogue.

3443. The device of item 3412 wherein the agent is a nitrogen mustard or an analogue or derivative thereof.

3444. The device of item 3412 wherein the agent is a hydroxyurea.

3445. The device of item 3412 wherein the agent is a mytomicin or an analogue or derivative thereof.

3446. The device of item 3412 wherein the agent is an alkyl sulfonate.

3447. The device of item 3412 wherein the agent is a benzamide or an analogue or derivative thereof.

3448. The device of item 3412 wherein the agent is a nicotinamide or an analogue or derivative thereof.

3449. The device of item 3412 wherein the agent is a halogenated sugar or an analogue or derivative thereof.

3450. The device of item 3412 wherein the agent is a DNA alkylating agent.

3451. The device of item 3412 wherein the agent is an anti-microtubule agent.

3452. The device of item 3412 wherein the agent is a topoisomerase inhibitor.

3453. The device of item 3412 wherein the agent is a DNA cleaving agent.

3454. The device of item 3412 wherein the agent is an antimetabolite.

3455. The device of item 3412 wherein the agent inhibits adenosine deaminase.

3456. The device of item 3412 wherein the agent inhibits purine ring synthesis.

3457. The device of item 3412 wherein the agent is a nucleotide interconversion inhibitor.

3458. The device of item 3412 wherein the agent inhibits dihydrofolate reduction.

3459. The device of item 3412 wherein the agent blocks thymidine monophosphate.

3460. The device of item 3412 wherein the agent causes DNA damage.

3461. The device of item 3412 wherein the agent is a DNA intercalation agent.

3462. The device of item 3412 wherein the agent is a RNA synthesis inhibitor.

3463. The device of item 3412 wherein the agent is a pyrimidine synthesis inhibitor.

3464. The device of item 3412 wherein the agent inhibits ribonucleotide synthesis or function.

3465. The device of item 3412 wherein the agent inhibits thymidine monophosphate synthesis or function.

3466. The device of item 3412 wherein the agent inhibits DNA synthesis.

3467. The device of item 3412 wherein the agent causes DNA adduct formation.

3468. The device of item 3412 wherein the agent inhibits protein synthesis.

3469. The device of item 3412 wherein the agent inhibits microtubule function.

3470. The device of item 3412 wherein the agent is a cyclin dependent protein kinase inhibitor.

3471. The device of item 3412 wherein the agent is an epidermal growth factor kinase inhibitor.

3472. The device of item 3412 wherein the agent is an elastase inhibitor.

3473. The device of item 3412 wherein the agent is a factor Xa inhibitor.

3474. The device of item 3412 wherein the agent is a farnesyltransferase inhibitor.

3475. The device of item 3412 wherein the agent is a fibrinogen antagonist.

3476. The device of item 3412 wherein the agent is a guanylate cyclase stimulant.

3477. The device of item 3412 wherein the agent is a heat shock protein 90 antagonist.

3478. The device of item 3412 wherein the agent is a heat shock protein 90 antagonist, wherein the heat shock protein 90 antagonist is geldanamycin or an analogue or derivative thereof.

3479. The device of item 3412 wherein the agent is a guanylate cyclase stimulant.

3480. The device of item 3412 wherein the agent is a HMGCoA reductase inhibitor.

3481. The device of item 3412 wherein the agent is a HMGCoA reductase inhibitor, wherein the HMGCoA reductase inhibitor is simvastatin or an analogue or derivative thereof.

3482. The device of item 3412 wherein the agent is a hydroorotate dehydrogenase inhibitor.

3483. The device of item 3412 wherein the agent is an IKK2 inhibitor.

3484. The device of item 3412 wherein the agent is an IL-1 antagonist.

3485. The device of item 3412 wherein the agent is an ICE antagonist.

3486. The device of item 3412 wherein the agent is an IRAK antagonist.

3487. The device of item 3412 wherein the agent is an IL-4 agonist.

3488. The device of item 3412 wherein the agent is an immunomodulatory agent.

3489. The device of item 3412 wherein the agent is sirolimus or an analogue or derivative thereof.

3490. The device of item 3412 wherein the agent is not sirolimus.

3491. The device of item 3412 wherein the agent is everolimus or an analogue or derivative thereof.

3492. The device of item 3412 wherein the agent is tacrolimus or an analogue or derivative thereof.

3493. The device of item 3412 wherein the agent is not tacrolimus.

3494. The device of item 3412 wherein the agent is biolmus or an analogue or derivative thereof.

3495. The device of item 3412 wherein the agent is tresperimus or an analogue or derivative thereof.

3496. The device of item 3412 wherein the agent is auranofin or an analogue or derivative thereof.

3497. The device of item 3412 wherein the agent is 27-0-demethylrapamycin or an analogue or derivative thereof.

3498. The device of item 3412 wherein the agent is gusperimus or an analogue or derivative thereof.

3499. The device of item 3412 wherein the agent is pimecrolimus or an analogue or derivative thereof.

3500. The device of item 3412 wherein the agent is ABT-578 or an analogue or derivative thereof.

3501. The device of item 3412 wherein the agent is an inosine monophosphate dehydrogenase (IMPDH) inhibitor.

3502. The device of item 3412 wherein the agent is an IMPDH inhibitor, wherein the IMPDH inhibitor is mycophenolic acid or an analogue or derivative thereof.

3503. The device of item 3412 wherein the agent is an IMPDH inhibitor, wherein the IMPDH inhibitor is 1-alpha-25 dihydroxy vitamin D3 or an analogue or derivative thereof.

3504. The device of item 3412 wherein the agent is a leukotriene inhibitor.

3505. The device of item 3412 wherein the agent is a MCP-1 antagonist.

3506. The device of item 3412 wherein the agent is a MMP inhibitor.

3507. The device of item 3412 wherein the agent is an NF kappa B inhibitor.

3508. The device of item 3412 wherein the agent is an NF kappa B inhibitor, wherein the NF kappa B inhibitor is Bay 11-7082.

3509. The device of item 3412 wherein the agent is an NO agonist.

3510. The device of item 3412 wherein the agent is a p38 MAP kinase inhibitor.

3511. The device of item 3412 wherein the agent is a p38 MAP kinase inhibitor, wherein the p38 MAP kinase inhibitor is SB 202190.

3512. The device of item 3412 wherein the agent is a phosphodiesterase inhibitor.

3513. The device of item 3412 wherein the agent is a TGF beta inhibitor.

3514. The device of item 3412 wherein the agent is a thromboxane A2 antagonist.

3515. The device of item 3412 wherein the agent is a TNFa antagonist.

3516. The device of item 3412 wherein the agent is a TACE inhibitor.

3517. The device of item 3412 wherein the agent is a tyrosine kinase inhibitor.

3518. The device of item 3412 wherein the agent is a vitronectin inhibitor.

3519. The device of item 3412 wherein the agent is a fibroblast growth factor inhibitor.

3520. The device of item 3412 wherein the agent is a protein kinase inhibitor.

3521. The device of item 3412 wherein the agent is a PDGF receptor kinase inhibitor.

3522. The device of item 3412 wherein the agent is an endothelial growth factor receptor kinase inhibitor.

3523. The device of item 3412 wherein the agent is a retinoic acid receptor antagonist.

3524. The device of item 3412 wherein the agent is a platelet derived growth factor receptor kinase inhibitor.

3525. The device of item 3412 wherein the agent is a fibronogin antagonist.

3526. The device of item 3412 wherein the agent is an antimycotic agent.

3527. The device of item 3412 wherein the agent is an antimycotic agent, wherein the antimycotic agent is sulconizole.

3528. The device of item 3412 wherein the agent is a bisphosphonate.

3529. The device of item 3412 wherein the agent is a phospholipase A1 inhibitor.

3530. The device of item 3412 wherein the agent is a histamine H1/H2/H3 receptor antagonist.

3531. The device of item 3412 wherein the agent is a macrolide antibiotic.

3532. The device of item 3412 wherein the agent is a GPIIb/IIIa receptor antagonist.

3533. The device of item 3412 wherein the agent is an endothelin receptor antagonist.

3534. The device of item 3412 wherein the agent is a peroxisome proliferator-activated receptor agonist.

3535. The device of item 3412 wherein the agent is an estrogen receptor agent.

3536. The device of item 3412 wherein the agent is a somastostatin analogue.

3537. The device of item 3412 wherein the agent is a neurokinin 1 antagonist.

3538. The device of item 3412 wherein the agent is a neurokinin 3 antagonist.

3539. The device of item 3412 wherein the agent is a VLA-4 antagonist.

3540. The device of item 3412 wherein the agent is an osteoclast inhibitor.

3541. The device of item 3412 wherein the agent is a DNA topoisomerase ATP hydrolyzing inhibitor.

3542. The device of item 3412 wherein the agent is an angiotensin I converting enzyme inhibitor.

3543. The device of item 3412 wherein the agent is an angiotensin II antagonist.

3544. The device of item 3412 wherein the agent is an enkephalinase inhibitor.

3545. The device of item 3412 wherein the agent is a peroxisome proliferator-activated receptor gamma agonist insulin sensitizer.

3546. The device of item 3412 wherein the agent is a protein kinase C inhibitor.

3547. The device of item 3412 wherein the agent is a ROCK (rho-associated kinase) inhibitor.

3548. The device of item 3412 wherein the agent is a CXCR3 inhibitor.

3549. The device of item 3412 wherein the agent is an Itk inhibitor.

3550. The device of item 3412 wherein the agent is a cytosolic phospholipase A2-alpha inhibitor.

3551. The device of item 3412 wherein the agent is a PPAR agonist.

3552. The device of item 3412 wherein the agent is an immunosuppressant.

3553. The device of item 3412 wherein the agent is an Erb inhibitor.

3554. The device of item 3412 wherein the agent is an apoptosis agonist.

3555. The device of item 3412 wherein the agent is a lipocortin agonist.

3556. The device of item 3412 wherein the agent is a VCAM-1 antagonist.

3557. The device of item 3412 wherein the agent is a collagen antagonist.

3558. The device of item 3412 wherein the agent is an alpha 2 integrin antagonist.

3559. The device of item 3412 wherein the agent is a TNF alpha inhibitor.

3560. The device of item 3412 wherein the agent is a nitric oxide inhibitor.

3561. The device of item 3412 wherein the agent is a cathepsin inhibitor.

3562. The device of item 3412 wherein the agent is not an anti-inflammatory agent.

3563. The device of item 3412 wherein the agent is not a steroid.

3564. The device of item 3412 wherein the agent is not a glucocorticosteroid.

3565. The device of item 3412 wherein the agent is not dexamethasone.

3566. The device of item 3412 wherein the agent is not an anti-infective agent.

3567. The device of item 3412 wherein the agent is not an antibiotic.

3568. The device of item 3412 wherein the agent is not an anti-fungal agent.

3569. The device of item 3412, further comprising a polymer.

3570. The device of item 3412, further comprising a polymeric carrier.

3571. The device of item 3412 wherein the anti-scarring agent inhibits adhesion between the device and a host into which the device is implanted.

3572. The device of item 3412 wherein the device delivers the anti-scarring agent locally to tissue proximate to the device.

3573. The device of item 3412, further comprising a coating, wherein the coating comprises the anti-scarring agent.

3574. The device of item 3412, further comprising a coating, wherein the coating is disposed on a surface of the device.

3575. The device of item 3412, further comprising a coating, wherein the coating directly contacts the device.

3576. The device of item 3412, further comprising a coating, wherein the coating indirectly contacts the device.

3577. The device of item 3412, further comprising a coating, wherein the coating partially covers the device.

3578. The device of item 3412, further comprising a coating, wherein the coating completely covers the device.

3579. The device of item 3412, further comprising a coating, wherein the coating is a uniform coating.

3580. The device of item 3412, further comprising a coating, wherein the coating is a non-uniform coating.

3581. The device of item 3412, further comprising a coating, wherein the coating is a discontinuous coating.

3582. The device of item 3412, further comprising a coating, wherein the coating is a patterned coating.

3583. The device of item 3412, further comprising a coating, wherein the coating has a thickness of 100 μm or less.

3584. The device of item 3412, further comprising a coating, wherein the coating has a thickness of 10 μm or less.

3585. The device of item 3412, further comprising a coating, wherein the coating adheres to the surface of the device upon deployment of the device.

3586. The device of item 3412, further comprising a coating, wherein the coating is stable at room temperature for a period of 1 year.

3587. The device of item 3412, further comprising a coating, wherein the anti-scarring agent is present in the coating in an amount ranging between about 0.0001% to about 1% by weight.

3588. The device of item 3412, further comprising a coating, wherein the anti-scarring agent is present in the coating in an amount ranging between about 1% to about 10% by weight.

3589. The device of item 3412, further comprising a coating, wherein the anti-scarring agent is present in the coating in an amount ranging between about 10% to about 25% by weight.

3590. The device of item 3412, further comprising a coating, wherein the anti-scarring agent is present in the coating in an amount ranging between about 25% to about 70% by weight.

3591. The device of item 3412, further comprising a coating, wherein the coating further comprises a polymer.

3592. The device of item 3412, further comprising a first coating having a first composition and the second coating having a second composition.

3593. The device of item 3412, further comprising a first coating having a first composition and the second coating having a second composition, wherein the first composition and the second composition are different.

3594. The device of item 3412, further comprising a polymer.

3595. The device of item 3412, further comprising a polymeric carrier.

3596. The device of item 3412, further comprising a polymeric carrier, wherein the polymeric carrier comprises a copolymer.

3597. The device of item 3412, further comprising a polymeric carrier, wherein the polymeric carrier comprises a block copolymer.

3598. The device of item 3412, further comprising a polymeric carrier, wherein the polymeric carrier comprises a random copolymer.

3599. The device of item 3412, further comprising a polymeric carrier, wherein the polymeric carrier comprises a biodegradable polymer.

3600. The device of item 3412, further comprising a polymeric carrier, wherein the polymeric carrier comprises a non-biodegradable polymer.

3601. The device of item 3412, further comprising a polymeric carrier, wherein the polymeric carrier comprises a hydrophilic polymer.

3602. The device of item 3412, further comprising a polymeric carrier, wherein the polymeric carrier comprises a hydrophobic polymer.

3603. The device of item 3412, further comprising a polymeric carrier, wherein the polymeric carrier comprises a polymer having hydrophilic domains.

3604. The device of item 3412, further comprising a polymeric carrier, wherein the polymeric carrier comprises a polymer having hydrophobic domains.

3605. The device of item 3412, further comprising a polymeric carrier, wherein the polymeric carrier comprises a non-conductive polymer.

3606. The device of item 3412, further comprising a polymeric carrier, wherein the polymeric carrier comprises an elastomer.

3607. The device of item 3412, further comprising a polymeric carrier, wherein the polymeric carrier comprises a hydrogel.

3608. The device of item 3412, further comprising a polymeric carrier, wherein the polymeric carrier comprises a silicone polymer.

3609. The device of item 3412, further comprising a polymeric carrier, wherein the polymeric carrier comprises a hydrocarbon polymer.

3610. The device of item 3412, further comprising a polymeric carrier, wherein the polymeric carrier comprises a styrene-derived polymer.

3611. The device of item 3412, further comprising a polymeric carrier, wherein the polymeric carrier comprises a butadiene polymer.

3612. The device of item 3412, further comprising a polymeric carrier, wherein the polymeric carrier comprises a macromer.

3613. The device of item 3412, further comprising a polymeric carrier, wherein the polymeric carrier comprises a poly(ethylene glycol)polymer.

3614. The device of item 3412, further comprising a polymeric carrier, wherein the polymeric carrier comprises an amorphous polymer.

3615. The device of item 3412, further comprising a lubricious coating.

3616. The device of item 3412 wherein the anti-scarring agent is located within pores or holes of the device.

3617. The device of item 3412 wherein the anti-scarring agent is located within a channel, lumen, or divet of the device.

3618. The device of item 3412, further comprising a second pharmaceutically active agent.

3619. The device of item 3412, further comprising an anti-inflammatory agent.

3620. The device of item 3412, further comprising an agent that inhibits infection.

3621. The device of item 3412, further comprising an agent that inhibits infection, wherein the agent is an anthracycline.

3622. The device of item 3412, further comprising an agent that inhibits infection, wherein the agent is doxorubicin.

3623. The device of item 3412, further comprising an agent that inhibits infection, wherein the agent is mitoxantrone.

3624. The device of item 3412, further comprising an agent that inhibits infection, wherein the agent is a fluoropyrimidine.

3625. The device of item 3412, further comprising an agent that inhibits infection, wherein the agent is 5-fluorouracil (5-FU).

3626. The device of item 3412, further comprising an agent that inhibits infection, wherein the agent is a folic acid antagonist.

3627. The device of item 3412, further comprising an agent that inhibits infection, wherein the agent is methotrexate.

3628. The device of item 3412, further comprising an agent that inhibits infection, wherein the agent is a podophylotoxin.

3629. The device of item 3412, further comprising an agent that inhibits infection, wherein the agent is etoposide.

3630. The device of item 3412, further comprising an agent that inhibits infection, wherein the agent is a camptothecin.

3631. The device of item 3412, further comprising an agent that inhibits infection, wherein the agent is a hydroxyurea.

3632. The device of item 3412, further comprising an agent that inhibits infection, wherein the agent is a platinum complex.

3633. The device of item 3412, further comprising an agent that inhibits infection, wherein the agent is cisplatin.

3634. The device of item 3412, further comprising an anti-thrombotic agent.

3635. The device of item 3412, further comprising a visualization agent.

3636. The device of item 3412, further comprising a visualization agent, wherein the visualization agent is a radiopaque material, wherein the radiopaque material comprises a metal, a halogenated compound, or a barium containing compound.

3637. The device of item 3412, further comprising a visualization agent, wherein the visualization agent is a radiopaque material, wherein the radiopaque material comprises barium, tantalum, or technetium.

3638. The device of item 3412, further comprising a visualization agent, wherein the visualization agent is a MRI responsive material.

3639. The device of item 3412, further comprising a visualization agent, wherein the visualization agent comprises a gadolinium chelate.

3640. The device of item 3412, further comprising a visualization agent, wherein the visualization agent comprises iron, magnesium, manganese, copper, or chromium.

3641. The device of item 3412, further comprising a visualization agent, wherein the visualization agent comprises an iron oxide compound.

3642. The device of item 3412, further comprising a visualization agent, wherein the visualization agent comprises a dye, pigment, or colorant.

3643. The device of item 3412, further comprising an echogenic material.

3644. The device of item 3412, further comprising an echogenic material, wherein the echogenic material is in the form of a coating.

3645. The device of item 3412 wherein the device is sterile.

3646. The device of item 3412 wherein the anti-scarring agent is released into tissue in the vicinity of the device after deployment of the device.

3647. The device of item 3412 wherein the anti-scarring agent is released into tissue in the vicinity of the device after deployment of the device, wherein the tissue is connective tissue.

3648. The device of item 3412 wherein the anti-scarring agent is released into tissue in the vicinity of the device after deployment of the device, wherein the tissue is muscle tissue.

3649. The device of item 3412 wherein the anti-scarring agent is released into tissue in the vicinity of the device after deployment of the device, wherein the tissue is nerve tissue.

3650. The device of item 3412 wherein the anti-scarring agent is released into tissue in the vicinity of the device after deployment of the device, wherein the tissue is epithelium tissue.

3651. The device of item 3412 wherein the anti-scarring agent is released in effective concentrations from the device over a period ranging from the time of deployment of the device to about 1 year.

3652. The device of item 3412 wherein the anti-scarring agent is released in effective concentrations from the device over a period ranging from about 1 month to 6 months.

3653. The device of item 3412 wherein the anti-scarring agent is released in effective concentrations from the device over a period ranging from about 1-90 days.

3654. The device of item 3412 wherein the anti-scarring agent is released in effective concentrations from the device at a constant rate.

3655. The device of item 3412 wherein the anti-scarring agent is released in effective concentrations from the device at an increasing rate.

3656. The device of item 3412 wherein the anti-scarring agent is released in effective concentrations from the device at a decreasing rate.

3657. The device of item 3412 wherein the anti-scarring agent is released in effective concentrations from the composition comprising the anti-scarring agent by diffusion over a period ranging from the time of deployment of the device to about 90 days.

3658. The device of item 3412 wherein the anti-scarring agent is released in effective concentrations from the composition comprising the anti-scarring agent by erosion of the composition over a period ranging from the time of deployment of the device to about 90 days.

3659. The device of item 3412 wherein the device comprises about 0.01 μg to about 10 μg of the anti-scarring agent.

3660. The device of item 3412 wherein the device comprises about 10 μg to about 10 mg of the anti-scarring agent.

3661. The device of item 3412 wherein the device comprises about 10 mg to about 250 mg of the anti-scarring agent.

3662. The device of item 3412 wherein the device comprises about 250 mg to about 1000 mg of the anti-scarring agent.

3663. The device of item 3412 wherein the device comprises about 1000 mg to about 2500 mg of the anti-scarring agent.

3664. The device of item 3412 wherein a surface of the device comprises less than 0.01 μg of the anti-scarring agent per mm2 of device surface to which the anti-scarring agent is applied.

3665. The device of item 3412 wherein a surface of the device comprises about 0.01 μg to about 1 μg of the anti-scarring agent per mm2 of device surface to which the anti-scarring agent is applied.

3666. The device of item 3412 wherein a surface of the device comprises about 1 μg to about 10 μg of the anti-scarring agent per mm2 of device surface to which the anti-scarring agent is applied.

3667. The device of item 3412 wherein a surface of the device comprises about 10 μg to about 250 μg of the anti-scarring agent per mm2 of device surface to which the anti-scarring agent is applied.

3668. The device of item 3412 wherein a surface of the device comprises about 250 μg to about 1000 μg of the anti-scarring agent of anti-scarring agent per mm2 of device surface to which the anti-scarring agent is applied.

3669. The device of item 3412 wherein a surface of the device comprises about 1000 μg to about 2500 μg of the anti-scarring agent per mm2 of device surface to which the anti-scarring agent is applied.

3670. A device, comprising a gastrointestinal device (i.e., an implant) and an anti-scarring agent or a composition comprising an anti-scarring agent, wherein the agent inhibits scarring between the device and a host into which the device is implanted.

3671. The device of item 3670 wherein the agent inhibits cell regeneration.

3672. The device of item 3670 wherein the agent inhibits angiogenesis.

3673. The device of item 3670 wherein the agent inhibits fibroblast migration.

3674. The device of item 3670 wherein the agent inhibits fibroblast proliferation.

3675. The device of item 3670 wherein the agent inhibits deposition of extracellular matrix.

3676. The device of item 3670 wherein the agent inhibits tissue remodeling.

3677. The device of item 3670 wherein the agent is an angiogenesis inhibitor.

3678. The device of item 3670 wherein the agent is a 5-lipoxygenase inhibitor or antagonist.

3679. The device of item 3670 wherein the agent is a chemokine receptor antagonist.

3680. The device of item 3670 wherein the agent is a cell cycle inhibitor.

3681. The device of item 3670 wherein the agent is a taxane.

3682. The device of item 3670 wherein the agent is an anti-microtubule agent.

3683. The device of item 3670 wherein the agent is paclitaxel.

3684. The device of item 3670 wherein the agent is not paclitaxel.

3685. The device of item 3670 wherein the agent is an analogue or derivative of paclitaxel.

3686. The device of item 3670 wherein the agent is a vinca alkaloid.

3687. The device of item 3670 wherein the agent is camptothecin or an analogue or derivative thereof.

3688. The device of item 3670 wherein the agent is a podophyllotoxin.

3689. The device of item 3670 wherein the agent is a podophyllotoxin, wherein the podophyllotoxin is etoposide or an analogue or derivative thereof.

3690. The device of item 3670 wherein the agent is an anthracycline.

3691. The device of item 3670 wherein the agent is an anthracycline, wherein the anthracycline is doxorubicin or an analogue or derivative thereof.

3692. The device of item 3670 wherein the agent is an anthracycline, wherein the anthracycline is mitoxantrone or an analogue or derivative thereof.

3693. The device of item 3670 wherein the agent is a platinum compound.

3694. The device of item 3670 wherein the agent is a nitrosourea.

3695. The device of item 3670 wherein the agent is a nitroimidazole.

3696. The device of item 3670 wherein the agent is a folic acid antagonist.

3697. The device of item 3670 wherein the agent is a cytidine analogue.

3698. The device of item 3670 wherein the agent is a pyrimidine analogue.

3699. The device of item 3670 wherein the agent is a fluoropyrimidine analogue.

3700. The device of item 3670 wherein the agent is a purine analogue.

3701. The device of item 3670 wherein the agent is a nitrogen mustard or an analogue or derivative thereof.

3702. The device of item 3670 wherein the agent is a hydroxyurea.

3703. The device of item 3670 wherein the agent is a mytomicin or an analogue or derivative thereof.

3704. The device of item 3670 wherein the agent is an alkyl sulfonate.

3705. The device of item 3670 wherein the agent is a benzamide or an analogue or derivative thereof.

3706. The device of item 3670 wherein the agent is a nicotinamide or an analogue or derivative thereof.

3707. The device of item 3670 wherein the agent is a halogenated sugar or an analogue or derivative thereof.

3708. The device of item 3670 wherein the agent is a DNA alkylating agent.

3709. The device of item 3670 wherein the agent is an anti-microtubule agent.

3710. The device of item 3670 wherein the agent is a topoisomerase inhibitor.

3711. The device of item 3670 wherein the agent is a DNA cleaving agent.

3712. The device of item 3670 wherein the agent is an antimetabolite.

3713. The device of item 3670 wherein the agent inhibits adenosine deaminase.

3714. The device of item 3670 wherein the agent inhibits purine ring synthesis.

3715. The device of item 3670 wherein the agent is a nucleotide interconversion inhibitor.

3716. The device of item 3670 wherein the agent inhibits dihydrofolate reduction.

3717. The device of item 3670 wherein the agent blocks thymidine mono phosphate.

3718. The device of item 3670 wherein the agent causes DNA damage.

3719. The device of item 3670 wherein the agent is a DNA intercalation agent.

3720. The device of item 3670 wherein the agent is a RNA synthesis inhibitor.

3721. The device of item 3670 wherein the agent is a pyrimidine synthesis inhibitor.

3722. The device of item 3670 wherein the agent inhibits ribonucleotide synthesis or function.

3723. The device of item 3670 wherein the agent inhibits thymidine monophosphate synthesis or function.

3724. The device of item 3670 wherein the agent inhibits DNA synthesis.

3725. The device of item 3670 wherein the agent causes DNA adduct formation.

3726. The device of item 3670 wherein the agent inhibits protein synthesis.

3727. The device of item 3670 wherein the agent inhibits microtubule function.

3728. The device of item 3670 wherein the agent is a cyclin dependent protein kinase inhibitor.

3729. The device of item 3670 wherein the agent is an epidermal growth factor kinase inhibitor.

3730. The device of item 3670 wherein the agent is an elastase inhibitor.

3731. The device of item 3670 wherein the agent is a factor Xa inhibitor.

3732. The device of item 3670 wherein the agent is a farnesyltransferase inhibitor.

3733. The device of item 3670 wherein the agent is a fibrinogen antagonist.

3734. The device of item 3670 wherein the agent is a guanylate cyclase stimulant.

3735. The device of item 3670 wherein the agent is a heat shock protein 90 antagonist.

3736. The device of item 3670 wherein the agent is a heat shock protein 90 antagonist, wherein the heat shock protein 90 antagonist is geldanamycin or an analogue or derivative thereof.

3737. The device of item 3670 wherein the agent is a guanylate cyclase stimulant.

3738. The device of item 3670 wherein the agent is a HMGCoA reductase inhibitor.

3739. The device of item 3670 wherein the agent is a HMGCoA reductase inhibitor, wherein the HMGCoA reductase inhibitor is simvastatin or an analogue or derivative thereof.

3740. The device of item 3670 wherein the agent is a hydroorotate dehydrogenase inhibitor.

3741. The device of item 3670 wherein the agent is an IKK2 inhibitor.

3742. The device of item 3670 wherein the agent is an IL-1 antagonist.

3743. The device of item 3670 wherein the agent is an ICE antagonist.

3744. The device of item 3670 wherein the agent is an IRAK antagonist.

3745. The device of item 3670 wherein the agent is an IL-4 agonist.

3746. The device of item 3670 wherein the agent is an immunomodulatory agent.

3747. The device of item 3670 wherein the agent is sirolimus or an analogue or derivative thereof.

3748. The device of item 3670 wherein the agent is not sirolimus.

3749. The device of item 3670 wherein the agent is everolimus or an analogue or derivative thereof.

3750. The device of item 3670 wherein the agent is tacrolimus or an analogue or derivative thereof.

3751. The device of item 3670 wherein the agent is not tacrolimus.

3752. The device of item 3670 wherein the agent is biolmus or an analogue or derivative thereof.

3753. The device of item 3670 wherein the agent is tresperimus or an analogue or derivative thereof.

3754. The device of item 3670 wherein the agent is auranofin or an analogue or derivative thereof.

3755. The device of item 3670 wherein the agent is 27-0-demethylrapamycin or an analogue or derivative thereof.

3756. The device of item 3670 wherein the agent is gusperimus or an analogue or derivative thereof.

3757. The device of item 3670 wherein the agent is pimecrolimus or an analogue or derivative thereof.

3758. The device of item 3670 wherein the agent is ABT-578 or an analogue or derivative thereof.

3759. The device of item 3670 wherein the agent is an inosine monophosphate dehydrogenase (IMPDH) inhibitor.

3760. The device of item 3670 wherein the agent is an IMPDH inhibitor, wherein the IMPDH inhibitor is mycophenolic acid or an analogue or derivative thereof.

3761. The device of item 3670 wherein the agent is an IMPDH inhibitor, wherein the IMPDH inhibitor is 1-alpha-25 dihydroxy vitamin D3 or an analogue or derivative thereof.

3762. The device of item 3670 wherein the agent is a leukotriene inhibitor.

3763. The device of item 3670 wherein the agent is a MCP-1 antagonist.

3764. The device of item 3670 wherein the agent is a MMP inhibitor.

3765. The device of item 3670 wherein the agent is an NF kappa B inhibitor.

3766. The device of item 3670 wherein the agent is an NF kappa B inhibitor, wherein the NF kappa B inhibitor is Bay 11-7082.

3767. The device of item 3670 wherein the agent is an NO agonist.

3768. The device of item 3670 wherein the agent is a p38 MAP kinase inhibitor.

3769. The device of item 3670 wherein the agent is a p38 MAP kinase inhibitor, wherein the p38 MAP kinase inhibitor is SB 202190.

3770. The device of item 3670 wherein the agent is a phosphodiesterase inhibitor.

3771. The device of item 3670 wherein the agent is a TGF beta inhibitor.

3772. The device of item 3670 wherein the agent is a thromboxane A2 antagonist.

3773. The device of item 3670 wherein the agent is a TNFa antagonist.

3774. The device of item 3670 wherein the agent is a TACE inhibitor.

3775. The device of item 3670 wherein the agent is a tyrosine kinase inhibitor.

3776. The device of item 3670 wherein the agent is a vitronectin inhibitor.

3777. The device of item 3670 wherein the agent is a fibroblast growth factor inhibitor.

3778. The device of item 3670 wherein the agent is a protein kinase inhibitor.

3779. The device of item 3670 wherein the agent is a PDGF receptor kinase inhibitor.

3780. The device of item 3670 wherein the agent is an endothelial growth factor receptor kinase inhibitor.

3781. The device of item 3670 wherein the agent is a retinoic acid receptor antagonist.

3782. The device of item 3670 wherein the agent is a platelet derived growth factor receptor kinase inhibitor.

3783. The device of item 3670 wherein the agent is a fibronogin antagonist.

3784. The device of item 3670 wherein the agent is an antimycotic agent.

3785. The device of item 3670 wherein the agent is an antimycotic agent, wherein the antimycotic agent is sulconizole.

3786. The device of item 3670 wherein the agent is a bisphosphonate.

3787. The device of item 3670 wherein the agent is a phospholipase A1 inhibitor.

3788. The device of item 3670 wherein the agent is a histamine H1/H2/H3 receptor antagonist.

3789. The device of item 3670 wherein the agent is a macrolide antibiotic.

3790. The device of item 3670 wherein the agent is a GPIIb/IIIa receptor antagonist.

3791. The device of item 3670 wherein the agent is an endothelin receptor antagonist.

3792. The device of item 3670 wherein the agent is a peroxisome proliferator-activated receptor agonist.

3793. The device of item 3670 wherein the agent is an estrogen receptor agent.

3794. The device of item 3670 wherein the agent is a somastostatin analogue.

3795. The device of item 3670 wherein the agent is a neurokinin 1 antagonist.

3796. The device of item 3670 wherein the agent is a neurokinin 3 antagonist.

3797. The device of item 3670 wherein the agent is a VLA-4 antagonist.

3798. The device of item 3670 wherein the agent is an osteoclast inhibitor.

3799. The device of item 3670 wherein the agent is a DNA topoisomerase ATP hydrolyzing inhibitor.

3800. The device of item 3670 wherein the agent is an angiotensin I converting enzyme inhibitor.

3801. The device of item 3670 wherein the agent is an angiotensin II antagonist.

3802. The device of item 3670 wherein the agent is an enkephalinase inhibitor.

3803. The device of item 3670 wherein the agent is a peroxisome proliferator-activated receptor gamma agonist insulin sensitizer.

3804. The device of item 3670 wherein the agent is a protein kinase C inhibitor.

3805. The device of item 3670 wherein the agent is a ROCK (rho-associated kinase) inhibitor.

3806. The device of item 3670 wherein the agent is a CXCR3 inhibitor.

3807. The device of item 3670 wherein the agent is an Itk inhibitor.

3808. The device of item 3670 wherein the agent is a cytosolic phospholipase A2-alpha inhibitor.

3809. The device of item 3670 wherein the agent is a PPAR agonist.

3810. The device of item 3670 wherein the agent is an immunosuppressant.

3811. The device of item 3670 wherein the agent is an Erb inhibitor.

3812. The device of item 3670 wherein the agent is an apoptosis agonist.

3813. The device of item 3670 wherein the agent is a lipocortin agonist.

3814. The device of item 3670 wherein the agent is a VCAM-1 antagonist.

3815. The device of item 3670 wherein the agent is a collagen antagonist.

3816. The device of item 3670 wherein the agent is an alpha 2 integrin antagonist.

3817. The device of item 3670 wherein the agent is a TNF alpha inhibitor.

3818. The device of item 3670 wherein the agent is a nitric oxide inhibitor.

3819. The device of item 3670 wherein the agent is a cathepsin inhibitor.

3820. The device of item 3670 wherein the agent is not an anti-inflammatory agent.

3821. The device of item 3670 wherein the agent is not a steroid.

3822. The device of item 3670 wherein the agent is not a glucocorticosteroid.

3823. The device of item 3670 wherein the agent is not dexamethasone.

3824. The device of item 3670 wherein the agent is not an anti-infective agent.

3825. The device of item 3670 wherein the agent is not an antibiotic.

3826. The device of item 3670 wherein the agent is not an anti-fungal agent.

3827. The device of item 3670, further comprising a polymer.

3828. The device of item 3670, further comprising a polymeric carrier.

3829. The device of item 3670 wherein the anti-scarring agent inhibits adhesion between the device and a host into which the device is implanted.

3830. The device of item 3670 wherein the device delivers the anti-scarring agent locally to tissue proximate to the device.

3831. The device of item 3670, further comprising a coating, wherein the coating comprises the anti-scarring agent.

3832. The device of item 3670, further comprising a coating, wherein the coating is disposed on a surface of the device.

3833. The device of item 3670, further comprising a coating, wherein the coating directly contacts the device.

3834. The device of item 3670, further comprising a coating, wherein the coating indirectly contacts the device.

3835. The device of item 3670, further comprising a coating, wherein the coating partially covers the device.

3836. The device of item 3670, further comprising a coating, wherein the coating completely covers the device.

3837. The device of item 3670, further comprising a coating, wherein the coating is a uniform coating.

3838. The device of item 3670, further comprising a coating, wherein the coating is a non-uniform coating.

3839. The device of item 3670, further comprising a coating, wherein the coating is a discontinuous coating.

3840. The device of item 3670, further comprising a coating, wherein the coating is a patterned coating.

3841. The device of item 3670, further comprising a coating, wherein the coating has a thickness of 100 μm or less.

3842. The device of item 3670, further comprising a coating, wherein the coating has a thickness of 10 μm or less.

3843. The device of item 3670, further comprising a coating, wherein the coating adheres to the surface of the device upon deployment of the device.

3844. The device of item 3670, further comprising a coating, wherein the coating is stable at room temperature for a period of 1 year.

3845. The device of item 3670, further comprising a coating, wherein the anti-scarring agent is present in the coating in an amount ranging between about 0.0001% to about 1% by weight.

3846. The device of item 3670, further comprising a coating, wherein the anti-scarring agent is present in the coating in an amount ranging between about 1% to about 10% by weight.

3847. The device of item 3670, further comprising a coating, wherein the anti-scarring agent is present in the coating in an amount ranging between about 10% to about 25% by weight.

3848. The device of item 3670, further comprising a coating, wherein the anti-scarring agent is present in the coating in an amount ranging between about 25% to about 70% by weight.

3849. The device of item 3670, further comprising a coating, wherein the coating further comprises a polymer.

3850. The device of item 3670, further comprising a first coating having a first composition and the second coating having a second composition.

3851. The device of item 3670, further comprising a first coating having a first composition and the second coating having a second composition, wherein the first composition and the second composition are different.

3852. The device of item 3670, further comprising a polymer.

3853. The device of item 3670, further comprising a polymeric carrier.

3854. The device of item 3670, further comprising a polymeric carrier, wherein the polymeric carrier comprises a copolymer.

3855. The device of item 3670, further comprising a polymeric carrier, wherein the polymeric carrier comprises a block copolymer.

3856. The device of item 3670, further comprising a polymeric carrier, wherein the polymeric carrier comprises a random copolymer.

3857. The device of item 3670, further comprising a polymeric carrier, wherein the polymeric carrier comprises a biodegradable polymer.

3858. The device of item 3670, further comprising a polymeric carrier, wherein the polymeric carrier comprises a non-biodegradable polymer.

3859. The device of item 3670, further comprising a polymeric carrier, wherein the polymeric carrier comprises a hydrophilic polymer.

3860. The device of item 3670, further comprising a polymeric carrier, wherein the polymeric carrier comprises a hydrophobic polymer.

3861. The device of item 3670, further comprising a polymeric carrier, wherein the polymeric carrier comprises a polymer having hydrophilic domains.

3862. The device of item 3670, further comprising a polymeric carrier, wherein the polymeric carrier comprises a polymer having hydrophobic domains.

3863. The device of item 3670, further comprising a polymeric carrier, wherein the polymeric carrier comprises a non-conductive polymer.

3864. The device of item 3670, further comprising a polymeric carrier, wherein the polymeric carrier comprises an elastomer.

3865. The device of item 3670, further comprising a polymeric carrier, wherein the polymeric carrier comprises a hydrogel.

3866. The device of item 3670, further comprising a polymeric carrier, wherein the polymeric carrier comprises a silicone polymer.

3867. The device of item 3670, further comprising a polymeric carrier, wherein the polymeric carrier comprises a hydrocarbon polymer.

3868. The device of item 3670, further comprising a polymeric carrier, wherein the polymeric carrier comprises a styrene-derived polymer.

3869. The device of item 3670, further comprising a polymeric carrier, wherein the polymeric carrier comprises a butadiene polymer.

3870. The device of item 3670, further comprising a polymeric carrier, wherein the polymeric carrier comprises a macromer.

3871. The device of item 3670, further comprising a polymeric carrier, wherein the polymeric carrier comprises a poly(ethylene glycol)polymer.

3872. The device of item 3670, further comprising a polymeric carrier, wherein the polymeric carrier comprises an amorphous polymer.

3873. The device of item 3670, further comprising a lubricious coating.

3874. The device of item 3670 wherein the anti-scarring agent is located within pores or holes of the device.

3875. The device of item 3670 wherein the anti-scarring agent is located within a channel, lumen, or divet of the device.

3876. The device of item 3670, further comprising a second pharmaceutically active agent.

3877. The device of item 3670, further comprising an anti-inflammatory agent.

3878. The device of item 3670, further comprising an agent that inhibits infection.

3879. The device of item 3670, further comprising an agent that inhibits infection, wherein the agent is an anthracycline.

3880. The device of item 3670, further comprising an agent that inhibits infection, wherein the agent is doxorubicin.

3881. The device of item 3670, further comprising an agent that inhibits infection, wherein the agent is mitoxantrone.

3882. The device of item 3670, further comprising an agent that inhibits infection, wherein the agent is a fluoropyrimidine.

3883. The device of item 3670, further comprising an agent that inhibits infection, wherein the agent is 5-fluorouracil (5-FU).

3884. The device of item 3670, further comprising an agent that inhibits infection, wherein the agent is a folic acid antagonist.

3885. The device of item 3670, further comprising an agent that inhibits infection, wherein the agent is methotrexate.

3886. The device of item 3670, further comprising an agent that inhibits infection, wherein the agent is a podophylotoxin.

3887. The device of item 3670, further comprising an agent that inhibits infection, wherein the agent is etoposide.

3888. The device of item 3670, further comprising an agent that inhibits infection, wherein the agent is a camptothecin.

3889. The device of item 3670, further comprising an agent that inhibits infection, wherein the agent is a hydroxyurea.

3890. The device of item 3670, further comprising an agent that inhibits infection, wherein the agent is a platinum complex.

3891. The device of item 3670, further comprising an agent that inhibits infection, wherein the agent is cisplatin.

3892. The device of item 3670, further comprising an anti-thrombotic agent.

3893. The device of item 3670, further comprising a visualization agent.

3894. The device of item 3670, further comprising a visualization agent, wherein the visualization agent is a radiopaque material, wherein the radiopaque material comprises a metal, a halogenated compound, or a barium containing compound.

3895. The device of item 3670, further comprising a visualization agent, wherein the visualization agent is a radiopaque material, wherein the radiopaque material comprises barium, tantalum, or technetium.

3896. The device of item 3670, further comprising a visualization agent, wherein the visualization agent is a MRI responsive material.

3897. The device of item 3670, further comprising a visualization agent, wherein the visualization agent comprises a gadolinium chelate.

3898. The device of item 3670, further comprising a visualization agent, wherein the visualization agent comprises iron, magnesium, manganese, copper, or chromium.

3899. The device of item 3670, further comprising a visualization agent, wherein the visualization agent comprises an iron oxide compound.

3900. The device of item 3670, further comprising a visualization agent, wherein the visualization agent comprises a dye, pigment, or colorant.

3901. The device of item 3670, further comprising an echogenic material.

3902. The device of item 3670, further comprising an echogenic material, wherein the echogenic material is in the form of a coating.

3903. The device of item 3670 wherein the device is sterile.

3904. The device of item 3670 wherein the anti-scarring agent is released into tissue in the vicinity of the device after deployment of the device.

3905. The device of item 3670 wherein the anti-scarring agent is released into tissue in the vicinity of the device after deployment of the device, wherein the tissue is connective tissue.

3906. The device of item 3670 wherein the anti-scarring agent is released into tissue in the vicinity of the device after deployment of the device, wherein the tissue is muscle tissue.

3907. The device of item 3670 wherein the anti-scarring agent is released into tissue in the vicinity of the device after deployment of the device, wherein the tissue is nerve tissue.

3908. The device of item 3670 wherein the anti-scarring agent is released into tissue in the vicinity of the device after deployment of the device, wherein the tissue is epithelium tissue.

3909. The device of item 3670 wherein the anti-scarring agent is released in effective concentrations from the device over a period ranging from the time of deployment of the device to about 1 year.

3910. The device of item 3670 wherein the anti-scarring agent is released in effective concentrations from the device over a period ranging from about 1 month to 6 months.

3911. The device of item 3670 wherein the anti-scarring agent is released in effective concentrations from the device over a period ranging from about 1-90 days.

3912. The device of item 3670 wherein the anti-scarring agent is released in effective concentrations from the device at a constant rate.

3913. The device of item 3670 wherein the anti-scarring agent is released in effective concentrations from the device at an increasing rate.

3914. The device of item 3670 wherein the anti-scarring agent is released in effective concentrations from the device at a decreasing rate.

3915. The device of item 3670 wherein the anti-scarring agent is released in effective concentrations from the composition comprising the anti-scarring agent by diffusion over a period ranging from the time of deployment of the device to about 90 days.

3916. The device of item 3670 wherein the anti-scarring agent is released in effective concentrations from the composition comprising the anti-scarring agent by erosion of the composition over a period ranging from the time of deployment of the device to about 90 days.

3917. The device of item 3670 wherein the device comprises about 0.01 μg to about 10 μg of the anti-scarring agent.

3918. The device of item 3670 wherein the device comprises about 10 μg to about 10 mg of the anti-scarring agent.

3919. The device of item 3670 wherein the device comprises about 10 mg to about 250 mg of the anti-scarring agent.

3920. The device of item 3670 wherein the device comprises about 250 mg to about 1000 mg of the anti-scarring agent.

3921. The device of item 3670 wherein the device comprises about 1000 mg to about 2500 mg of the anti-scarring agent.

3922. The device of item 3670 wherein a surface of the device comprises less than 0.01 μg of the anti-scarring agent per mm2 of device surface to which the anti-scarring agent is applied.

3923. The device of item 3670 wherein a surface of the device comprises about 0.01 μg to about 1 μg of the anti-scarring agent per mm2 of device surface to which the anti-scarring agent is applied.

3924. The device of item 3670 wherein a surface of the device comprises about 1 μg to about 10 μg of the anti-scarring agent per mm2 of device surface to which the anti-scarring agent is applied.

3925. The device of item 3670 wherein a surface of the device comprises about 10 μg to about 250 μg of the anti-scarring agent per mm2 of device surface to which the anti-scarring agent is applied.

3926. The device of item 3670 wherein a surface of the device comprises about 250 μg to about 1000 μg of the anti-scarring agent of anti-scarring agent per mm2 of device surface to which the anti-scarring agent is applied.

3927. The device of item 3670 wherein a surface of the device comprises about 1000 μg to about 2500 μg of the anti-scarring agent per mm2 of device surface to which the anti-scarring agent is applied.

3928. The device of any one of items 3670-3927 wherein the implant is a GI tube for drainage.

3929. The device of any one of items 3670-3927 wherein the implant is a GI tube for feeding.

3930. The device of any one of items 3670-3927 wherein the implant is a portosystemic shunt.

3931. The device of any one of items 3670-3927 wherein the implant is a shunt for ascites.

3932. The device of any one of items 3670-3927 wherein the implant is a nasogastric tube.

3933. The device of any one of items 3670-3927 wherein the implant is a nasoenteral tube.

3934. The device of any one of items 3670-3927 wherein the implant is a gastrostomy feeding tube.

3935. The device of any one of items 3670-3927 wherein the implant is a percutaneous feeding tube.

3936. The device of any one of items 3670-3927 wherein the implant is a colostomy device.

3937. The device of any one of items 3670-3927 wherein the implant is a biliary T-tube.

3938. The device of any one of items 3670-3927 wherein the implant is a biliary stone removal device.

3939. The device of any one of items 3670-3927 wherein the implant is a dilation balloon.

3940. The device of any one of items 3670-3927 wherein the implant is a dilation catheter.

3941. The device of any one of items 3670-3927 wherein the implant is an enteral feeding device.

3942. The device of any one of items 3670-3927 wherein the implant is an esophageal stent.

3943. The device of any one of items 3670-3927 wherein the implant is a biliary stent.

3944. The device of any one of items 3670-3927 wherein the implant is a pancreatic stent.

3945. A device, comprising a spinal implant and an anti-scarring agent or a composition comprising an anti-scarring agent, wherein the agent inhibits scarring between the device and a host into which the device is implanted.

3946. The device of item 3945 wherein the agent inhibits cell regeneration.

3947. The device of item 3945 wherein the agent inhibits angiogenesis.

3948. The device of item 3945 wherein the agent inhibits fibroblast migration.

3949. The device of item 3945 wherein the agent inhibits fibroblast proliferation.

3950. The device of item 3945 wherein the agent inhibits deposition of extracellular matrix.

3951. The device of item 3945 wherein the agent inhibits tissue remodeling.

3952. The device of item 3945 wherein the agent is an angiogenesis inhibitor.

3953. The device of item 3945 wherein the agent is a 5-lipoxygenase inhibitor or antagonist.

3954. The device of item 3945 wherein the agent is a chemokine receptor antagonist.

3955. The device of item 3945 wherein the agent is a cell cycle inhibitor.

3956. The device of item 3945 wherein the agent is a taxane.

3957. The device of item 3945 wherein the agent is an anti-microtubule agent.

3958. The device of item 3945 wherein the agent is paclitaxel.

3959. The device of item 3945 wherein the agent is not paclitaxel.

3960. The device of item 3945 wherein the agent is an analogue or derivative of paclitaxel.

3961. The device of item 3945 wherein the agent is a vinca alkaloid.

3962. The device of item 3945 wherein the agent is camptothecin or an analogue or derivative thereof.

3963. The device of item 3945 wherein the agent is a podophyllotoxin.

3964. The device of item 3945 wherein the agent is a podophyllotoxin, wherein the podophyllotoxin is etoposide or an analogue or derivative thereof.

3965. The device of item 3945 wherein the agent is an anthracycline.

3966. The device of item 3945 wherein the agent is an anthracycline, wherein the anthracycline is doxorubicin or an analogue or derivative thereof.

3967. The device of item 3945 wherein the agent is an anthracycline, wherein the anthracycline is mitoxantrone or an analogue or derivative thereof.

3968. The device of item 3945 wherein the agent is a platinum compound.

3969. The device of item 3945 wherein the agent is a nitrosourea.

3970. The device of item 3945 wherein the agent is a nitroimidazole.

3971. The device of item 3945 wherein the agent is a folic acid antagonist.

3972. The device of item 3945 wherein the agent is a cytidine analogue.

3973. The device of item 3945 wherein the agent is a pyrimidine analogue.

3974. The device of item 3945 wherein the agent is a fluoropyrimidine analogue.

3975. The device of item 3945 wherein the agent is a purine analogue.

3976. The device of item 3945 wherein the agent is a nitrogen mustard or an analogue or derivative thereof.

3977. The device of item 3945 wherein the agent is a hydroxyurea.

3978. The device of item 3945 wherein the agent is a mytomicin or an analogue or derivative thereof.

3979. The device of item 3945 wherein the agent is an alkyl sulfonate.

3980. The device of item 3945 wherein the agent is a benzamide or an analogue or derivative thereof.

3981. The device of item 3945 wherein the agent is a nicotinamide or an analogue or derivative thereof.

3982. The device of item 3945 wherein the agent is a halogenated sugar or an analogue or derivative thereof.

3983. The device of item 3945 wherein the agent is a DNA alkylating agent.

3984. The device of item 3945 wherein the agent is an anti-microtubule agent.

3985. The device of item 3945 wherein the agent is a topoisomerase inhibitor.

3986. The device of item 3945 wherein the agent is a DNA cleaving agent.

3987. The device of item 3945 wherein the agent is an antimetabolite.

3988. The device of item 3945 wherein the agent inhibits adenosine deaminase.

3989. The device of item 3945 wherein the agent inhibits purine ring synthesis.

3990. The device of item 3945 wherein the agent is a nucleotide interconversion inhibitor.

3991. The device of item 3945 wherein the agent inhibits dihydrofolate reduction.

3992. The device of item 3945 wherein the agent blocks thymidine mono phosphate.

3993. The device of item 3945 wherein the agent causes DNA damage.

3994. The device of item 3945 wherein the agent is a DNA intercalation agent.

3995. The device of item 3945 wherein the agent is a RNA synthesis inhibitor.

3996. The device of item 3945 wherein the agent is a pyrimidine synthesis inhibitor.

3997. The device of item 3945 wherein the agent inhibits ribonucleotide synthesis or function.

3998. The device of item 3945 wherein the agent inhibits thymidine monophosphate synthesis or function.

3999. The device of item 3945 wherein the agent inhibits DNA synthesis.

4000. The device of item 3945 wherein the agent causes DNA adduct formation.

4001. The device of item 3945 wherein the agent inhibits protein synthesis.

4002. The device of item 3945 wherein the agent inhibits microtubule function.

4003. The device of item 3945 wherein the agent is a cyclin dependent protein kinase inhibitor.

4004. The device of item 3945 wherein the agent is an epidermal growth factor kinase inhibitor.

4005. The device of item 3945 wherein the agent is an elastase inhibitor.

4006. The device of item 3945 wherein the agent is a factor Xa inhibitor.

4007. The device of item 3945 wherein the agent is a farnesyltransferase inhibitor.

4008. The device of item 3945 wherein the agent is a fibrinogen antagonist.

4009. The device of item 3945 wherein the agent is a guanylate cyclase stimulant.

4010. The device of item 3945 wherein the agent is a heat shock protein 90 antagonist.

4011. The device of item 3945 wherein the agent is a heat shock protein 90 antagonist, wherein the heat shock protein 90 antagonist is geldanamycin or an analogue or derivative thereof.

4012. The device of item 3945 wherein the agent is a guanylate cyclase stimulant.

4013. The device of item 3945 wherein the agent is a HMGCoA reductase inhibitor.

4014. The device of item 3945 wherein the agent is a HMGCoA reductase inhibitor, wherein the HMGCoA reductase inhibitor is simvastatin or an analogue or derivative thereof.

4015. The device of item 3945 wherein the agent is a hydroorotate dehydrogenase inhibitor.

4016. The device of item 3945 wherein the agent is an IKK2 inhibitor.

4017. The device of item 3945 wherein the agent is an IL-1 antagonist.

4018. The device of item 3945 wherein the agent is an ICE antagonist.

4019. The device of item 3945 wherein the agent is an IRAK antagonist.

4020. The device of item 3945 wherein the agent is an IL-4 agonist.

4021. The device of item 3945 wherein the agent is an immunomodulatory agent.

4022. The device of item 3945 wherein the agent is sirolimus or an analogue or derivative thereof.

4023. The device of item 3945 wherein the agent is not sirolimus.

4024. The device of item 3945 wherein the agent is everolimus or an analogue or derivative thereof.

4025. The device of item 3945 wherein the agent is tacrotimus or an analogue or derivative thereof.

4026. The device of item 3945 wherein the agent is not tacrolimus.

4027. The device of item 3945 wherein the agent is biolmus or an analogue or derivative thereof.

4028. The device of item 3945 wherein the agent is tresperimus or an analogue or derivative thereof.

4029. The device of item 3945 wherein the agent is auranofin or an analogue or derivative thereof.

4030. The device of item 3945 wherein the agent is 27-0-demethylrapamycin or an analogue or derivative thereof.

4031. The device of item 3945 wherein the agent is gusperimus or an analogue or derivative thereof.

4032. The device of item 3945 wherein the agent is pimecrolimus or an analogue or derivative thereof.

4033. The device of item 3945 wherein the agent is ABT-578 or an analogue or derivative thereof.

4034. The device of item 3945 wherein the agent is an inosine monophosphate dehydrogenase (IMPDH) inhibitor.

4035. The device of item 3945 wherein the agent is an IMPDH inhibitor, wherein the IMPDH inhibitor is mycophenolic acid or an analogue or derivative thereof.

4036. The device of item 3945 wherein the agent is an IMPDH inhibitor, wherein the IMPDH inhibitor is 1-alpha-25 dihydroxy vitamin D3 or an analogue or derivative thereof.

4037. The device of item 3945 wherein the agent is a leukotriene inhibitor.

4038. The device of item 3945 wherein the agent is a MCP-1 antagonist.

4039. The device of item 3945 wherein the agent is a MMP inhibitor.

4040. The device of item 3945 wherein the agent is an NF kappa B inhibitor.

4041. The device of item 3945 wherein the agent is an NF kappa B inhibitor, wherein the NF kappa B inhibitor is Bay 11-7082.

4042. The device of item 3945 wherein the agent is an NO agonist.

4043. The device of item 3945 wherein the agent is a p38 MAP kinase inhibitor.

4044. The device of item 3945 wherein the agent is a p38 MAP kinase inhibitor, wherein the p38 MAP kinase inhibitor is SB 202190.

4045. The device of item 3945 wherein the agent is a phosphodiesterase inhibitor.

4046. The device of item 3945 wherein the agent is a TGF beta inhibitor.

4047. The device of item 3945 wherein the agent is a thromboxane A2 antagonist.

4048. The device of item 3945 wherein the agent is a TNFa antagonist.

4049. The device of item 3945 wherein the agent is a TACE inhibitor.

4050. The device of item 3945 wherein the agent is a tyrosine kinase inhibitor.

4051. The device of item 3945 wherein the agent is a vitronectin inhibitor.

4052. The device of item 3945 wherein the agent is a fibroblast growth factor inhibitor.

4053. The device of item 3945 wherein the agent is a protein kinase inhibitor.

4054. The device of item 3945 wherein the agent is a PDGF receptor kinase inhibitor.

4055. The device of item 3945 wherein the agent is an endothelial growth factor receptor kinase inhibitor.

4056. The device of item 3945 wherein the agent is a retinoic acid receptor antagonist.

4057. The device of item 3945 wherein the agent is a platelet derived growth factor receptor kinase inhibitor.

4058. The device of item 3945 wherein the agent is a fibronogin antagonist.

4059. The device of item 3945 wherein the agent is an antimycotic agent.

4060. The device of item 3945 wherein the agent is an antimycotic agent, wherein the antimycotic agent is sulconizole.

4061. The device of item 3945 wherein the agent is a bisphosphonate.

4062. The device of item 3945 wherein the agent is a phospholipase A1 inhibitor.

4063. The device of item 3945 wherein the agent is a histamine H1/H2/H3 receptor antagonist.

4064. The device of item 3945 wherein the agent is a macrolide antibiotic.

4065. The device of item 3945 wherein the agent is a GPIIb/IIIa receptor antagonist.

4066. The device of item 3945 wherein the agent is an endothelin receptor antagonist.

4067. The device of item 3945 wherein the agent is a peroxisome proliferator-activated receptor agonist.

4068. The device of item 3945 wherein the agent is an estrogen receptor agent.

4069. The device of item 3945 wherein the agent is a somastostatin analogue.

4070. The device of item 3945 wherein the agent is a neurokinin 1 antagonist.

4071. The device of item 3945 wherein the agent is a neurokinin 3 antagonist.

4072. The device of item 3945 wherein the agent is a VLA-4 antagonist.

4073. The device of item 3945 wherein the agent is an osteoclast inhibitor.

4074. The device of item 3945 wherein the agent is a DNA topoisomerase ATP hydrolyzing inhibitor.

4075. The device of item 3945 wherein the agent is an angiotensin I converting enzyme inhibitor.

4076. The device of item 3945 wherein the agent is an angiotensin II antagonist.

4077. The device of item 3945 wherein the agent is an enkephalinase inhibitor.

4078. The device of item 3945 wherein the agent is a peroxisome proliferator-activated receptor gamma agonist insulin sensitizer.

4079. The device of item 3945 wherein the agent is a protein kinase C inhibitor.

4080. The device of item 3945 wherein the agent is a ROCK (rho-associated kinase) inhibitor.

4081. The device of item 3945 wherein the agent is a CXCR3 inhibitor.

4082. The device of item 3945 wherein the agent is an Itk inhibitor.

4083. The device of item 3945 wherein the agent is a cytosolic phospholipase A2-alpha inhibitor.

4084. The device of item 3945 wherein the agent is a PPAR agonist.

4085. The device of item 3945 wherein the agent is an immunosuppressant.

4086. The device of item 3945 wherein the agent is an Erb inhibitor.

4087. The device of item 3945 wherein the agent is an apoptosis agonist.

4088. The device of item 3945 wherein the agent is a lipocortin agonist.

4089. The device of item 3945 wherein the agent is a VCAM-1 antagonist.

4090. The device of item 3945 wherein the agent is a collagen antagonist.

4091. The device of item 3945 wherein the agent is an alpha 2 integrin antagonist.

4092. The device of item 3945 wherein the agent is a TNF alpha inhibitor.

4093. The device of item 3945 wherein the agent is a nitric oxide inhibitor.

4094. The device of item 3945 wherein the agent is a cathepsin inhibitor.

4095. The device of item 3945 wherein the agent is not an anti-inflammatory agent.

4096. The device of item 3945 wherein the agent is not a steroid.

4097. The device of item 3945 wherein the agent is not a glucocorticosteroid.

4098. The device of item 3945 wherein the agent is not dexamethasone.

4099. The device of item 3945 wherein the agent is not an anti-infective agent.

4100. The device of item 3945 wherein the agent is not an antibiotic.

4101. The device of item 3945 wherein the agent is not an anti-fungal agent.

4102. The device of item 3945, further comprising a polymer.

4103. The device of item 3945, further comprising a polymeric carrier.

4104. The device of item 3945 wherein the anti-scarring agent inhibits adhesion between the device and a host into which the device is implanted.

4105. The device of item 3945 wherein the device delivers the anti-scarring agent locally to tissue proximate to the device.

4106. The device of item 3945, further comprising a coating, wherein the coating comprises the anti-scarring agent.

4107. The device of item 3945, further comprising a coating, wherein the coating is disposed on a surface of the device.

4108. The device of item 3945, further comprising a coating, wherein the coating directly contacts the device.

4109. The device of item 3945, further comprising a coating, wherein the coating indirectly contacts the device.

4110. The device of item 3945, further comprising a coating, wherein the coating partially covers the device.

4111. The device of item 3945, further comprising a coating, wherein the coating completely covers the device.

4112. The device of item 3945, further comprising a coating, wherein the coating is a uniform coating.

4113. The device of item 3945, further comprising a coating, wherein the coating is a non-uniform coating.

4114. The device of item 3945, further comprising a coating, wherein the coating is a discontinuous coating.

4115. The device of item 3945, further comprising a coating, wherein the coating is a patterned coating.

4116. The device of item 3945, further comprising a coating, wherein the coating has a thickness of 100 μm or less.

4117. The device of item 3945, further comprising a coating, wherein the coating has a thickness of 10 μm or less.

4118. The device of item 3945, further comprising a coating, wherein the coating adheres to the surface of the device upon deployment of the device.

4119. The device of item 3945, further comprising a coating, wherein the coating is stable at room temperature for a period of 1 year.

4120. The device of item 3945, further comprising a coating, wherein the anti-scarring agent is present in the coating in an amount ranging between about 0.0001% to about 1% by weight.

4121. The device of item 3945, further comprising a coating, wherein the anti-scarring agent is present in the coating in an amount ranging between about 1% to about 10% by weight.

4122. The device of item 3945, further comprising a coating, wherein the anti-scarring agent is present in the coating in an amount ranging between about 10% to about 25% by weight.

4123. The device of item 3945, further comprising a coating, wherein the anti-scarring agent is present in the coating in an amount ranging between about 25% to about 70% by weight.

4124. The device of item 3945, further comprising a coating, wherein the coating further comprises a polymer.

4125. The device of item 3945, further comprising a first coating having a first composition and the second coating having a second composition.

4126. The device of item 3945, further comprising a first coating having a first composition and the second coating having a second composition, wherein the first composition and the second composition are different.

4127. The device of item 3945, further comprising a polymer.

4128. The device of item 3945, further comprising a polymeric carrier.

4129. The device of item 3945, further comprising a polymeric carrier, wherein the polymeric carrier comprises a copolymer.

4130. The device of item 3945, further comprising a polymeric carrier, wherein the polymeric carrier comprises a block copolymer.

4131. The device of item 3945, further comprising a polymeric carrier, wherein the polymeric carrier comprises a random copolymer.

4132. The device of item 3945, further comprising a polymeric carrier, wherein the polymeric carrier comprises a biodegradable polymer.

4133. The device of item 3945, further comprising a polymeric carrier, wherein the polymeric carrier comprises a non-biodegradable polymer.

4134. The device of item 3945, further comprising a polymeric carrier, wherein the polymeric carrier comprises a hydrophilic polymer.

4135. The device of item 3945, further comprising a polymeric carrier, wherein the polymeric carrier comprises a hydrophobic polymer.

4136. The device of item 3945, further comprising a polymeric carrier, wherein the polymeric carrier comprises a polymer having hydrophilic domains.

4137. The device of item 3945, further comprising a polymeric carrier, wherein the polymeric carrier comprises a polymer having hydrophobic domains.

4138. The device of item 3945, further comprising a polymeric carrier, wherein the polymeric carrier comprises a non-conductive polymer.

4139. The device of item 3945, further comprising a polymeric carrier, wherein the polymeric carrier comprises an elastomer.

4140. The device of item 3945, further comprising a polymeric carrier, wherein the polymeric carrier comprises a hydrogel.

4141. The device of item 3945, further comprising a polymeric carrier, wherein the polymeric carrier comprises a silicone polymer.

4142. The device of item 3945, further comprising a polymeric carrier, wherein the polymeric carrier comprises a hydrocarbon polymer.

4143. The device of item 3945, further comprising a polymeric carrier, wherein the polymeric carrier comprises a styrene-derived polymer.

4144. The device of item 3945, further comprising a polymeric carrier, wherein the polymeric carrier comprises a butadiene polymer.

4145. The device of item 3945, further comprising a polymeric carrier, wherein the polymeric carrier comprises a macromer.

4146. The device of item 3945, further comprising a polymeric carrier, wherein the polymeric carrier comprises a poly(ethylene glycol)polymer.

4147. The device of item 3945, further comprising a polymeric carrier, wherein the polymeric carrier comprises an amorphous polymer.

4148. The device of item 3945, further comprising a lubricious coating.

4149. The device of item 3945 wherein the anti-scarring agent is located within pores or holes of the device.

4150. The device of item 3945 wherein the anti-scarring agent is located within a channel, lumen, or divet of the device.

4151. The device of item 3945, further comprising a second pharmaceutically active agent.

4152. The device of item 3945, further comprising an anti-inflammatory agent.

4153. The device of item 3945, further comprising an agent that inhibits infection.

4154. The device of item 3945, further comprising an agent that inhibits infection, wherein the agent is an anthracycline.

4155. The device of item 3945, further comprising an agent that inhibits infection, wherein the agent is doxorubicin.

4156. The device of item 3945, further comprising an agent that inhibits infection, wherein the agent is mitoxantrone.

4157. The device of item 3945, further comprising an agent that inhibits infection, wherein the agent is a fluoropyrimidine.

4158. The device of item 3945, further comprising an agent that inhibits infection, wherein the agent is 5-fluorouracil (5-FU).

4159. The device of item 3945, further comprising an agent that inhibits infection, wherein the agent is a folic acid antagonist.

4160. The device of item 3945, further comprising an agent that inhibits infection, wherein the agent is methotrexate.

4161. The device of item 3945, further comprising an agent that inhibits infection, wherein the agent is a podophylotoxin.

4162. The device of item 3945, further comprising an agent that inhibits infection, wherein the agent is etoposide.

4163. The device of item 3945, further comprising an agent that inhibits infection, wherein the agent is a camptothecin.

4164. The device of item 3945, further comprising an agent that inhibits infection, wherein the agent is a hydroxyurea.

4165. The device of item 3945, further comprising an agent that inhibits infection, wherein the agent is a platinum complex.

4166. The device of item 3945, further comprising an agent that inhibits infection, wherein the agent is cisplatin.

4167. The device of item 3945, further comprising an anti-thrombotic agent.

4168. The device of item 3945, further comprising a visualization agent.

4169. The device of item 3945, further comprising a visualization agent, wherein the visualization agent is a radiopaque material, wherein the radiopaque material comprises a metal, a halogenated compound, or a barium containing compound.

4170. The device of item 3945, further comprising a visualization agent, wherein the visualization agent is a radiopaque material, wherein the radiopaque material comprises barium, tantalum, or technetium.

4171. The device of item 3945, further comprising a visualization agent, wherein the visualization agent is a MRI responsive material.

4172. The device of item 3945, further comprising a visualization agent, wherein the visualization agent comprises a gadolinium chelate.

4173. The device of item 3945, further comprising a visualization agent, wherein the visualization agent comprises iron, magnesium, manganese, copper, or chromium.

4174. The device of item 3945, further comprising a visualization agent, wherein the visualization agent comprises an iron oxide compound.

4175. The device of item 3945, further comprising a visualization agent, wherein the visualization agent comprises a dye, pigment, or colorant.

4176. The device of item 3945, further comprising an echogenic material.

4177. The device of item 3945, further comprising an echogenic material, wherein the echogenic material is in the form of a coating.

4178. The device of item 3945 wherein the device is sterile.

4179. The device of item 3945 wherein the anti-scarring agent is released into tissue in the vicinity of the device after deployment of the device.

4180. The device of item 3945 wherein the anti-scarring agent is released into tissue in the vicinity of the device after deployment of the device, wherein the tissue is connective tissue.

4181. The device of item 3945 wherein the anti-scarring agent is released into tissue in the vicinity of the device after deployment of the device, wherein the tissue is muscle tissue.

4182. The device of item 3945 wherein the anti-scarring agent is released into tissue in the vicinity of the device after deployment of the device, wherein the tissue is nerve tissue.

4183. The device of item 3945 wherein the anti-scarring agent is released into tissue in the vicinity of the device after deployment of the device, wherein the tissue is epithelium tissue.

4184. The device of item 3945 wherein the anti-scarring agent is released in effective concentrations from the device over a period ranging from the time of deployment of the device to about 1 year.

4185. The device of item 3945 wherein the anti-scarring agent is released in effective concentrations from the device over a period ranging from about 1 month to 6 months.

4186. The device of item 3945 wherein the anti-scarring agent is released in effective concentrations from the device over a period ranging from about 1-90 days.

4187. The device of item 3945 wherein the anti-scarring agent is released in effective concentrations from the device at a constant rate.

4188. The device of item 3945 wherein the anti-scarring agent is released in effective concentrations from the device at an increasing rate.

4189. The device of item 3945 wherein the anti-scarring agent is released in effective concentrations from the device at a decreasing rate.

4190. The device of item 3945 wherein the anti-scarring agent is released in effective concentrations from the composition comprising the anti-scarring agent by diffusion over a period ranging from the time of deployment of the device to about 90 days.

4191. The device of item 3945 wherein the anti-scarring agent is released in effective concentrations from the composition comprising the anti-scarring agent by erosion of the composition over a period ranging from the time of deployment of the device to about 90 days.

4192. The device of item 3945 wherein the device comprises about 0.01 μg to about 10 μg of the anti-scarring agent.

4193. The device of item 3945 wherein the device comprises about 10 μg to about 10 mg of the anti-scarring agent.

4194. The device of item 3945 wherein the device comprises about 10 mg to about 250 mg of the anti-scarring agent.

4195. The device of item 3945 wherein the device comprises about 250 mg to about 1000 mg of the anti-scarring agent.

4196. The device of item 3945 wherein the device comprises about 1000 mg to about 2500 mg of the anti-scarring agent.

4197. The device of item 3945 wherein a surface of the device comprises less than 0.01 μg of the anti-scarring agent per mm2 of device surface to which the anti-scarring agent is applied.

4198. The device of item 3945 wherein a surface of the device comprises about 0.01 μg to about 1 μg of the anti-scarring agent per mm2 of device surface to which the anti-scarring agent is applied.

4199. The device of item 3945 wherein a surface of the device comprises about 1 μg to about 10 μg of the anti-scarring agent per mm2 of device surface to which the anti-scarring agent is applied.

4200. The device of item 3945 wherein a surface of the device comprises about 10 μg to about 250 μg of the anti-scarring agent per mm2 of device surface to which the anti-scarring agent is applied.

4201. The device of item 3945 wherein a surface of the device comprises about 250 μg to about 1000 μg of the anti-scarring agent of anti-scarring agent per mm2 of device surface to which the anti-scarring agent is applied.

4202. The device of item 3945 wherein a surface of the device comprises about 1000 μg to about 2500 μg of the anti-scarring agent per mm2 of device surface to which the anti-scarring agent is applied.

4203. The device of any one of items 3945-4202 wherein the implant is a spinal disc.

4204. The device of any one of items 3945-4202 wherein the implant is a vertebral disc prosthesis.

4205. The device of any one of items 3945-4202 wherein the implant is an intervertebral disc.

4206. The device of any one of items 3945-4202 wherein the implant is a partial spinal prosthesis.

4207. The device of any one of items 3945-4202 wherein the implant is a spinal nucleus implant.

4208. The device of any one of items 3945-4202 wherein the implant is an intervertebral disc spacer.

4209. The device of any one of items 3945-4202 wherein the implant is a fusion cage.

4210. The device of any one of items 3945-4202 wherein the implant is a fusion basket.

4211. The device of any one of items 3945-4202 wherein the implant is a fusion chamber.

4212. The device of any one of items 3945-4202 wherein the implant is a spinal anchoring device.

4213. The device of any one of items 3945-4202 wherein the implant is a bone fixation device.

4214. The device of any one of items 3945-4202 wherein the implant is an anchoring bone plate for the spine.

4215. The device of any one of items 3945-4202 wherein the implant is an anchoring screw for the spine.

4216. The device of any one of items 3945-4202 wherein the implant is an implantable rod for the spine.

4217. The device of any one of items 3945-4202 wherein the implant is an implantable dowel for the spine.

4218. The device of any one of items 3945-4202 wherein the implant is an implantable hook for the spine.

4219. The device of any one of items 3945-4202 wherein the implant is a wire for spinal binding.

4220. The device of any one of items 3945-4202 wherein the implant is a wedge for spinal support.

4221. A device, comprising a pressure monitoring implant and an anti-scarring agent or a composition comprising an anti-scarring agent, wherein the agent inhibits scarring between the device and a host into which the device is implanted.

4222. The device of item 4221 wherein the agent inhibits cell regeneration.

4223. The device of item 4221 wherein the agent inhibits angiogenesis.

4224. The device of item 4221 wherein the agent inhibits fibroblast migration.

4225. The device of item 4221 wherein the agent inhibits fibroblast proliferation.

4226. The device of item 4221 wherein the agent inhibits deposition of extracellular matrix.

4227. The device of item 4221 wherein the agent inhibits tissue remodeling.

4228. The device of item 4221 wherein the agent is an angiogenesis inhibitor.

4229. The device of item 4221 wherein the agent is a 5-lipoxygenase inhibitor or antagonist.

4230. The device of item 4221 wherein the agent is a chemokine receptor antagonist.

4231. The device of item 4221 wherein the agent is a cell cycle inhibitor.

4232. The device of item 4221 wherein the agent is a taxane.

4233. The device of item 4221 wherein the agent is an anti-microtubule agent.

4234. The device of item 4221 wherein the agent is paclitaxel.

4235. The device of item 4221 wherein the agent is not paclitaxel.

4236. The device of item 4221 wherein the agent is an analogue or derivative of paclitaxel.

4237. The device of item 4221 wherein the agent is a vinca alkaloid.

4238. The device of item 4221 wherein the agent is camptothecin or an analogue or derivative thereof.

4239. The device of item 4221 wherein the agent is a podophyllotoxin.

4240. The device of item 4221 wherein the agent is a podophyllotoxin, wherein the podophyllotoxin is etoposide or an analogue or derivative thereof.

4241. The device of item 4221 wherein the agent is an anthracycline.

4242. The device of item 4221 wherein the agent is an anthracycline, wherein the anthracycline is doxorubicin or an analogue or derivative thereof.

4243. The device of item 4221 wherein the agent is an anthracycline, wherein the anthracycline is mitoxantrone or an analogue or derivative thereof.

4244. The device of item 4221 wherein the agent is a platinum compound.

4245. The device of item 4221 wherein the agent is a nitrosourea.

4246. The device of item 4221 wherein the agent is a nitroimidazole.

4247. The device of item 4221 wherein the agent is a folic acid antagonist.

4248. The device of item 4221 wherein the agent is a cytidine analogue.

4249. The device of item 4221 wherein the agent is a pyrimidine analogue.

4250. The device of item 4221 wherein the agent is a fluoropyrimidine analogue.

4251. The device of item 4221 wherein the agent is a purine analogue.

4252. The device of item 4221 wherein the agent is a nitrogen mustard or an analogue or derivative thereof.

4253. The device of item 4221 wherein the agent is a hydroxyurea.

4254. The device of item 4221 wherein the agent is a mytomicin or an analogue or derivative thereof.

4255. The device of item 4221 wherein the agent is an alkyl sulfonate.

4256. The device of item 4221 wherein the agent is a benzamide or an analogue or derivative thereof.

4257. The device of item 4221 wherein the agent is a nicotinamide or an analogue or derivative thereof.

4258. The device of item 4221 wherein the agent is a halogenated sugar or an analogue or derivative thereof.

4259. The device of item 4221 wherein the agent is a DNA alkylating agent.

4260. The device of item 4221 wherein the agent is an anti-microtubule agent.

4261. The device of item 4221 wherein the agent is a topoisomerase inhibitor.

4262. The device of item 4221 wherein the agent is a DNA cleaving agent.

4263. The device of item 4221 wherein the agent is an antimetabolite.

4264. The device of item 4221 wherein the agent inhibits adenosine deaminase.

4265. The device of item 4221 wherein the agent inhibits purine ring synthesis.

4266. The device of item 4221 wherein the agent is a nucleotide interconversion inhibitor.

4267. The device of item 4221 wherein the agent inhibits dihydrofolate reduction.

4268. The device of item 4221 wherein the agent blocks thymidine monophosphate.

4269. The device of item 4221 wherein the agent causes DNA damage.

4270. The device of item 4221 wherein the agent is a DNA intercalation agent.

4271. The device of item 4221 wherein the agent is a RNA synthesis inhibitor.

4272. The device of item 4221 wherein the agent is a pyrimidine synthesis inhibitor.

4273. The device of item 4221 wherein the agent inhibits ribonucleotide synthesis or function.

4274. The device of item 4221 wherein the agent inhibits thymidine monophosphate synthesis or function.

4275. The device of item 4221 wherein the agent inhibits DNA synthesis.

4276. The device of item 4221 wherein the agent causes DNA adduct formation.

4277. The device of item 4221 wherein the agent inhibits protein synthesis.

4278. The device of item 4221 wherein the agent inhibits microtubule function.

4279. The device of item 4221 wherein the agent is a cyclin dependent protein kinase inhibitor.

4280. The device of item 4221 wherein the agent is an epidermal growth factor kinase inhibitor.

4281. The device of item 4221 wherein the agent is an elastase inhibitor.

4282. The device of item 4221 wherein the agent is a factor Xa inhibitor.

4283. The device of item 4221 wherein the agent is a farnesyltransferase inhibitor.

4284. The device of item 4221 wherein the agent is a fibrinogen antagonist.

4285. The device of item 4221 wherein the agent is a guanylate cyclase stimulant.

4286. The device of item 4221 wherein the agent is a heat shock protein 90 antagonist.

4287. The device of item 4221 wherein the agent is a heat shock protein 90 antagonist, wherein the heat shock protein 90 antagonist is geldanamycin or an analogue or derivative thereof.

4288. The device of item 4221 wherein the agent is a guanylate cyclase stimulant.

4289. The device of item 4221 wherein the agent is a HMGCoA reductase inhibitor.

4290. The device of item 4221 wherein the agent is a HMGCoA reductase inhibitor, wherein the HMGCoA reductase inhibitor is simvastatin or an analogue or derivative thereof.

4291. The device of item 4221 wherein the agent is a hydroorotate dehydrogenase inhibitor.

4292. The device of item 4221 wherein the agent is an IKK2 inhibitor.

4293. The device of item 4221 wherein the agent is an IL-1 antagonist.

4294. The device of item 4221 wherein the agent is an ICE antagonist.

4295. The device of item 4221 wherein the agent is an IRAK antagonist.

4296. The device of item 4221 wherein the agent is an IL-4 agonist.

4297. The device of item 4221 wherein the agent is an immunomodulatory agent.

4298. The device of item 4221 wherein the agent is sirolimus or an analogue or derivative thereof.

4299. The device of item 4221 wherein the agent is not sirolimus.

4300. The device of item 4221 wherein the agent is everolimus or an analogue or derivative thereof.

4301. The device of item 4221 wherein the agent is tacrolimus or an analogue or derivative thereof.

4302. The device of item 4221 wherein the agent is not tacrolimus.

4303. The device of item 4221 wherein the agent is biolmus or an analogue or derivative thereof.

4304. The device of item 4221 wherein the agent is tresperimus or an analogue or derivative thereof.

4305. The device of item 4221 wherein the agent is auranofin or an analogue or derivative thereof.

4306. The device of item 4221 wherein the agent is 27-0-demethylrapamycin or an analogue or derivative thereof.

4307. The device of item 4221 wherein the agent is gusperimus or an analogue or derivative thereof.

4308. The device of item 4221 wherein the agent is pimecrolimus or an analogue or derivative thereof.

4309. The device of item 4221 wherein the agent is ABT-578 or an analogue or derivative thereof.

4310. The device of item 4221 wherein the agent is an inosine monophosphate dehydrogenase (IMPDH) inhibitor.

4311. The device of item 4221 wherein the agent is an IMPDH inhibitor, wherein the IMPDH inhibitor is mycophenolic acid or an analogue or derivative thereof.

4312. The device of item 4221 wherein the agent is an IMPDH inhibitor, wherein the IMPDH inhibitor is 1-alpha-25 dihydroxy vitamin D3 or an analogue or derivative thereof.

4313. The device of item 4221 wherein the agent is a leukotriene inhibitor.

4314. The device of item 4221 wherein the agent is a MCP-1 antagonist.

4315. The device of item 4221 wherein the agent is a MMP inhibitor.

4316. The device of item 4221 wherein the agent is an NF kappa B inhibitor.

4317. The device of item 4221 wherein the agent is an NF kappa B inhibitor, wherein the NF kappa B inhibitor is Bay 11-7082.

4318. The device of item 4221 wherein the agent is an NO agonist.

4319. The device of item 4221 wherein the agent is a p38 MAP kinase inhibitor.

4320. The device of item 4221 wherein the agent is a p38 MAP kinase inhibitor, wherein the p38 MAP kinase inhibitor is SB 202190.

4321. The device of item 4221 wherein the agent is a phosphodiesterase inhibitor.

4322. The device of item 4221 wherein the agent is a TGF beta inhibitor.

4323. The device of item 4221 wherein the agent is a thromboxane A2 antagonist.

4324. The device of item 4221 wherein the agent is a TNFa antagonist.

4325. The device of item 4221 wherein the agent is a TACE inhibitor.

4326. The device of item 4221 wherein the agent is a tyrosine kinase inhibitor.

4327. The device of item 4221 wherein the agent is a vitronectin inhibitor.

4328. The device of item 4221 wherein the agent is a fibroblast growth factor inhibitor.

4329. The device of item 4221 wherein the agent is a protein kinase inhibitor.

4330. The device of item 4221 wherein the agent is a PDGF receptor kinase inhibitor.

4331. The device of item 4221 wherein the agent is an endothelial growth factor receptor kinase inhibitor.

4332. The device of item 4221 wherein the agent is a retinoic acid receptor antagonist.

4333. The device of item 4221 wherein the agent is a platelet derived growth factor receptor kinase inhibitor.

4334. The device of item 4221 wherein the agent is a fibronogin antagonist.

4335. The device of item 4221 wherein the agent is an antimycotic agent.

4336. The device of item 4221 wherein the agent is an antimycotic agent, wherein the antimycotic agent is sulconizole.

4337. The device of item 4221 wherein the agent is a bisphosphonate.

4338. The device of item 4221 wherein the agent is a phospholipase A1 inhibitor.

4339. The device of item 4221 wherein the agent is a histamine H1/H2/H3 receptor antagonist.

4340. The device of item 4221 wherein the agent is a macrolide antibiotic.

4341. The device of item 4221 wherein the agent is a GPIIb/IIIa receptor antagonist.

4342. The device of item 4221 wherein the agent is an endothelin receptor antagonist.

4343. The device of item 4221 wherein the agent is a peroxisome proliferator-activated receptor agonist.

4344. The device of item 4221 wherein the agent is an estrogen receptor agent.

4345. The device of item 4221 wherein the agent is a somastostatin analogue.

4346. The device of item 4221 wherein the agent is a neurokinin 1 antagonist.

4347. The device of item 4221 wherein the agent is a neurokinin 3 antagonist.

4348. The device of item 4221 wherein the agent is a VLA-4 antagonist.

4349. The device of item 4221 wherein the agent is an osteoclast inhibitor.

4350. The device of item 4221 wherein the agent is a DNA topoisomerase ATP hydrolyzing inhibitor.

4351. The device of item 4221 wherein the agent is an angiotensin I converting enzyme inhibitor.

4352. The device of item 4221 wherein the agent is an angiotensin II antagonist.

4353. The device of item 4221 wherein the agent is an enkephalinase inhibitor.

4354. The device of item 4221 wherein the agent is a peroxisome proliferator-activated receptor gamma agonist insulin sensitizer.

4355. The device of item 4221 wherein the agent is a protein kinase C inhibitor.

4356. The device of item 4221 wherein the agent is a ROCK (rho-associated kinase) inhibitor.

4357. The device of item 4221 wherein the agent is a CXCR3 inhibitor.

4358. The device of item 4221 wherein the agent is an Itk inhibitor.

4359. The device of item 4221 wherein the agent is a cytosolic phospholipase A2-alpha inhibitor.

4360. The device of item 4221 wherein the agent is a PPAR agonist.

4361. The device of item 4221 wherein the agent is an immunosuppressant.

4362. The device of item 4221 wherein the agent is an Erb inhibitor.

4363. The device of item 4221 wherein the agent is an apoptosis agonist.

4364. The device of item 4221 wherein the agent is a lipocortin agonist.

4365. The device of item 4221 wherein the agent is a VCAM-1 antagonist.

4366. The device of item 4221 wherein the agent is a collagen antagonist.

4367. The device of item 4221 wherein the agent is an alpha 2 integrin antagonist.

4368. The device of item 4221 wherein the agent is a TNF alpha inhibitor.

4369. The device of item 4221 wherein the agent is a nitric oxide inhibitor.

4370. The device of item 4221 wherein the agent is a cathepsin inhibitor.

4371. The device of item 4221 wherein the agent is not an anti-inflammatory agent.

4372. The device of item 4221 wherein the agent is not a steroid.

4373. The device of item 4221 wherein the agent is not a glucocorticosteroid.

4374. The device of item 4221 wherein the agent is not dexamethasone.

4375. The device of item 4221 wherein the agent is not an anti-infective agent.

4376. The device of item 4221 wherein the agent is not an antibiotic.

4377. The device of item 4221 wherein the agent is not an anti-fungal agent.

4378. The device of item 4221, further comprising a polymer.

4379. The device of item 4221, further comprising a polymeric carrier.

4380. The device of item 4221 wherein the anti-scarring agent inhibits adhesion between the device and a host into which the device is implanted.

4381. The device of item 4221 wherein the device delivers the anti-scarring agent locally to tissue proximate to the device.

4382. The device of item 4221, further comprising a coating, wherein the coating comprises the anti-scarring agent.

4383. The device of item 4221, further comprising a coating, wherein the coating is disposed on a surface of the device.

4384. The device of item 4221, further comprising a coating, wherein the coating directly contacts the device.

4385. The device of item 4221, further comprising a coating, wherein the coating indirectly contacts the device.

4386. The device of item 4221, further comprising a coating, wherein the coating partially covers the device.

4387. The device of item 4221, further comprising a coating, wherein the coating completely covers the device.

4388. The device of item 4221, further comprising a coating, wherein the coating is a uniform coating.

4389. The device of item 4221, further comprising a coating, wherein the coating is a non-uniform coating.

4390. The device of item 4221, further comprising a coating, wherein the coating is a discontinuous coating.

4391. The device of item 4221, further comprising a coating, wherein the coating is a patterned coating.

4392. The device of item 4221, further comprising a coating, wherein the coating has a thickness of 100 μm or less.

4393. The device of item 4221, further comprising a coating, wherein the coating has a thickness of 10 μm or less.

4394. The device of item 4221, further comprising a coating, wherein the coating adheres to the surface of the device upon deployment of the device.

4395. The device of item 4221, further comprising a coating, wherein the coating is stable at room temperature for a period of 1 year.

4396. The device of item 4221, further comprising a coating, wherein the anti-scarring agent is present in the coating in an amount ranging between about 0.0001% to about 1% by weight.

4397. The device of item 4221, further comprising a coating, wherein the anti-scarring agent is present in the coating in an amount ranging between about 1% to about 10% by weight.

4398. The device of item 4221, further comprising a coating, wherein the anti-scarring agent is present in the coating in an amount ranging between about 10% to about 25% by weight.

4399. The device of item 4221, further comprising a coating, wherein the anti-scarring agent is present in the coating in an amount ranging between about 25% to about 70% by weight.

4400. The device of item 4221, further comprising a coating, wherein the coating further comprises a polymer.

4401. The device of item 4221, further comprising a first coating having a first composition and the second coating having a second composition.

4402. The device of item 4221, further comprising a first coating having a first composition and the second coating having a second composition, wherein the first composition and the second composition are different.

4403. The device of item 4221, further comprising a polymer.

4404. The device of item 4221, further comprising a polymeric carrier.

4405. The device of item 4221, further comprising a polymeric carrier, wherein the polymeric carrier comprises a copolymer.

4406. The device of item 4221, further comprising a polymeric carrier, wherein the polymeric carrier comprises a block copolymer.

4407. The device of item 4221, further comprising a polymeric carrier, wherein the polymeric carrier comprises a random copolymer.

4408. The device of item 4221, further comprising a polymeric carrier, wherein the polymeric carrier comprises a biodegradable polymer.

4409. The device of item 4221, further comprising a polymeric carrier, wherein the polymeric carrier comprises a non-biodegradable polymer.

4410. The device of item 4221, further comprising a polymeric carrier, wherein the polymeric carrier comprises a hydrophilic polymer.

4411. The device of item 4221, further comprising a polymeric carrier, wherein the polymeric carrier comprises a hydrophobic polymer.

4412. The device of item 4221, further comprising a polymeric carrier, wherein the polymeric carrier comprises a polymer having hydrophilic domains.

4413. The device of item 4221, further comprising a polymeric carrier, wherein the polymeric carrier comprises a polymer having hydrophobic domains.

4414. The device of item 4221, further comprising a polymeric carrier, wherein the polymeric carrier comprises a non-conductive polymer.

4415. The device of item 4221, further comprising a polymeric carrier, wherein the polymeric carrier comprises an elastomer.

4416. The device of item 4221, further comprising a polymeric carrier, wherein the polymeric carrier comprises a hydrogel.

4417. The device of item 4221, further comprising a polymeric carrier, wherein the polymeric carrier comprises a silicone polymer.

4418. The device of item 4221, further comprising a polymeric carrier, wherein the polymeric carrier comprises a hydrocarbon polymer.

4419. The device of item 4221, further comprising a polymeric carrier, wherein the polymeric carrier comprises a styrene-derived polymer.

4420. The device of item 4221, further comprising a polymeric carrier, wherein the polymeric carrier comprises a butadiene polymer.

4421. The device of item 4221, further comprising a polymeric carrier, wherein the polymeric carrier comprises a macromer.

4422. The device of item 4221, further comprising a polymeric carrier, wherein the polymeric carrier comprises a poly(ethylene glycol)polymer.

4423. The device of item 4221, further comprising a polymeric carrier, wherein the polymeric carrier comprises an amorphous polymer.

4424. The device of item 4221, further comprising a lubricious coating.

4425. The device of item 4221 wherein the anti-scarring agent is located within pores or holes of the device.

4426. The device of item 4221 wherein the anti-scarring agent is located within a channel, lumen, or divet of the device.

4427. The device of item 4221, further comprising a second pharmaceutically active agent.

4428. The device of item 4221, further comprising an anti-inflammatory agent.

4429. The device of item 4221, further comprising an agent that inhibits infection.

4430. The device of item 4221, further comprising an agent that inhibits infection, wherein the agent is an anthracycline.

4431. The device of item 4221, further comprising an agent that inhibits infection, wherein the agent is doxorubicin.

4432. The device of item 4221, further comprising an agent that inhibits infection, wherein the agent is mitoxantrone.

4433. The device of item 4221, further comprising an agent that inhibits infection, wherein the agent is a fluoropyrimidine.

4434. The device of item 4221, further comprising an agent that inhibits infection, wherein the agent is 5-fluorouracil (5-FU).

4435. The device of item 4221, further comprising an agent that inhibits infection, wherein the agent is a folic acid antagonist.

4436. The device of item 4221, further comprising an agent that inhibits infection, wherein the agent is methotrexate.

4437. The device of item 4221, further comprising an agent that inhibits infection, wherein the agent is a podophylotoxin.

4438. The device of item 4221, further comprising an agent that inhibits infection, wherein the agent is etoposide.

4439. The device of item 4221, further comprising an agent that inhibits infection, wherein the agent is a camptothecin.

4440. The device of item 4221, further comprising an agent that inhibits infection, wherein the agent is a hydroxyurea.

4441. The device of item 4221, further comprising an agent that inhibits infection, wherein the agent is a platinum complex.

4442. The device of item 4221, further comprising an agent that inhibits infection, wherein the agent is cisplatin.

4443. The device of item 4221, further comprising an anti-thrombotic agent.

4444. The device of item 4221, further comprising a visualization agent.

4445. The device of item 4221, further comprising a visualization agent, wherein the visualization agent is a radiopaque material, wherein the radiopaque material comprises a metal, a halogenated compound, or a barium containing compound.

4446. The device of item 4221, further comprising a visualization agent, wherein the visualization agent is a radiopaque material, wherein the radiopaque material comprises barium, tantalum, or technetium.

4447. The device of item 4221, further comprising a visualization agent, wherein the visualization agent is a MRI responsive material.

4448. The device of item 4221, further comprising a visualization agent, wherein the visualization agent comprises a gadolinium chelate.

4449. The device of item 4221, further comprising a visualization agent, wherein the visualization agent comprises iron, magnesium, manganese, copper, or chromium.

4450. The device of item 4221, further comprising a visualization agent, wherein the visualization agent comprises an iron oxide compound.

4451. The device of item 4221, further comprising a visualization agent, wherein the visualization agent comprises a dye, pigment, or colorant.

4452. The device of item 4221, further comprising an echogenic material.

4453. The device of item 4221, further comprising an echogenic material, wherein the echogenic material is in the form of a coating.

4454. The device of item 4221 wherein the device is sterile.

4455. The device of item 4221 wherein the anti-scarring agent is released into tissue in the vicinity of the device after deployment of the device.

4456. The device of item 4221 wherein the anti-scarring agent is released into tissue in the vicinity of the device after deployment of the device, wherein the tissue is connective tissue.

4457. The device of item 4221 wherein the anti-scarring agent is released into tissue in the vicinity of the device after deployment of the device, wherein the tissue is muscle tissue.

4458. The device of item 4221 wherein the anti-scarring agent is released into tissue in the vicinity of the device after deployment of the device, wherein the tissue is nerve tissue.

4459. The device of item 4221 wherein the anti-scarring agent is released into tissue in the vicinity of the device after deployment of the device, wherein the tissue is epithelium tissue.

4460. The device of item 4221 wherein the anti-scarring agent is released in effective concentrations from the device over a period ranging from the time of deployment of the device to about 1 year.

4461. The device of item 4221 wherein the anti-scarring agent is released in effective concentrations from the device over a period ranging from about 1 month to 6 months.

4462. The device of item 4221 wherein the anti-scarring agent is released in effective concentrations from the device over a period ranging from about 1-90 days.

4463. The device of item 4221 wherein the anti-scarring agent is released in effective concentrations from the device at a constant rate.

4464. The device of item 4221 wherein the anti-scarring agent is released in effective concentrations from the device at an increasing rate.

4465. The device of item 4221 wherein the anti-scarring agent is released in effective concentrations from the device at a decreasing rate.

4466. The device of item 4221 wherein the anti-scarring agent is released in effective concentrations from the composition comprising the anti-scarring agent by diffusion over a period ranging from the time of deployment of the device to about 90 days.

4467. The device of item 4221 wherein the anti-scarring agent is released in effective concentrations from the composition comprising the anti-scarring agent by erosion of the composition over a period ranging from the time of deployment of the device to about 90 days.

4468. The device of item 4221 wherein the device comprises about 0.01 μg to about 10 μg of the anti-scarring agent.

4469. The device of item 4221 wherein the device comprises about 10 μg to about 10 mg of the anti-scarring agent.

4470. The device of item 4221 wherein the device comprises about 10 mg to about 250 mg of the anti-scarring agent.

4471. The device of item 4221 wherein the device comprises about 250 mg to about 1000 mg of the anti-scarring agent.

4472. The device of item 4221 wherein the device comprises about 1000 mg to about 2500 mg of the anti-scarring agent.

4473. The device of item 4221 wherein a surface of the device comprises less than 0.01 μg of the anti-scarring agent per mm2 of device surface to which the anti-scarring agent is applied.

4474. The device of item 4221 wherein a surface of the device comprises about 0.01 μg to about 1 μg of the anti-scarring agent per mm2 of device surface to which the anti-scarring agent is applied.

4475. The device of item 4221 wherein a surface of the device comprises about 1 μg to about 10 μg of the anti-scarring agent per mm2 of device surface to which the anti-scarring agent is applied.

4476. The device of item 4221 wherein a surface of the device comprises about 10 μg to about 250 μg of the anti-scarring agent per mm2 of device surface to which the anti-scarring agent is applied.

4477. The device of item 4221 wherein a surface of the device comprises about 250 μg to about 1000 μg of the anti-scarring agent of anti-scarring agent per mm2 of device surface to which the anti-scarring agent is applied.

4478. The device of item 4221 wherein a surface of the device comprises about 1000 μg to about 2500 μg of the anti-scarring agent per mm2 of device surface to which the anti-scarring agent is applied.

4479. A device, comprising a tympanostomy tube implant and an anti-scarring agent or a composition comprising an anti-scarring agent, wherein the agent inhibits scarring between the device and a host into which the device is implanted.

4480. The device of item 4479 wherein the agent inhibits cell regeneration.

4481. The device of item 4479 wherein the agent inhibits angiogenesis.

4482. The device of item 4479 wherein the agent inhibits fibroblast migration.

4483. The device of item 4479 wherein the agent inhibits fibroblast proliferation.

4484. The device of item 4479 wherein the agent inhibits deposition of extracellular matrix.

4485. The device of item 4479 wherein the agent inhibits tissue remodeling.

4486. The device of item 4479 wherein the agent is an angiogenesis inhibitor.

4487. The device of item 4479 wherein the agent is a 5-lipoxygenase inhibitor or antagonist.

4488. The device of item 4479 wherein the agent is a chemokine receptor antagonist.

4489. The device of item 4479 wherein the agent is a cell cycle inhibitor.

4490. The device of item 4479 wherein the agent is a taxane.

4491. The device of item 4479 wherein the agent is an anti-microtubule agent.

4492. The device of item 4479 wherein the agent is paclitaxel.

4493. The device of item 4479 wherein the agent is not paclitaxel.

4494. The device of item 4479 wherein the agent is an analogue or derivative of paclitaxel.

4495. The device of item 4479 wherein the agent is a vinca alkaloid.

4496. The device of item 4479 wherein the agent is camptothecin or an analogue or derivative thereof.

4497. The device of item 4479 wherein the agent is a podophyllotoxin.

4498. The device of item 4479 wherein the agent is a podophyllotoxin, wherein the podophyllotoxin is etoposide or an analogue or derivative thereof.

4499. The device of item 4479 wherein the agent is an anthracycline.

4500. The device of item 4479 wherein the agent is an anthracycline, wherein the anthracycline is doxorubicin or an analogue or derivative thereof.

4501. The device of item 4479 wherein the agent is an anthracycline, wherein the anthracycline is mitoxantrone or an analogue or derivative thereof.

4502. The device of item 4479 wherein the agent is a platinum compound.

4503. The device of item 4479 wherein the agent is a nitrosourea.

4504. The device of item 4479 wherein the agent is a nitroimidazole.

4505. The device of item 4479 wherein the agent is a folic acid antagonist.

4506. The device of item 4479 wherein the agent is a cytidine analogue.

4507. The device of item 4479 wherein the agent is a pyrimidine analogue.

4508. The device of item 4479 wherein the agent is a fluoropyrimidine analogue.

4509. The device of item 4479 wherein the agent is a purine analogue.

4510. The device of item 4479 wherein the agent is a nitrogen mustard or an analogue or derivative thereof.

4511. The device of item 4479 wherein the agent is a hydroxyurea.

4512. The device of item 4479 wherein the agent is a mytomicin or an analogue or derivative thereof.

4513. The device of item 4479 wherein the agent is an alkyl sulfonate.

4514. The device of item 4479 wherein the agent is a benzamide or an analogue or derivative thereof.

4515. The device of item 4479 wherein the agent is a nicotinamide or an analogue or derivative thereof.

4516. The device of item 4479 wherein the agent is a halogenated sugar or an analogue or derivative thereof.

4517. The device of item 4479 wherein the agent is a DNA alkylating agent.

4518. The device of item 4479 wherein the agent is an anti-microtubule agent.

4519. The device of item 4479 wherein the agent is a topoisomerase inhibitor.

4520. The device of item 4479 wherein the agent is a DNA cleaving agent.

4521. The device of item 4479 wherein the agent is an antimetabolite.

4522. The device of item 4479 wherein the agent inhibits adenosine deaminase.

4523. The device of item 4479 wherein the agent inhibits purine ring synthesis.

4524. The device of item 4479 wherein the agent is a nucleotide interconversion inhibitor.

4525. The device of item 4479 wherein the agent inhibits dihydrofolate reduction.

4526. The device of item 4479 wherein the agent blocks thymidine monophosphate.

4527. The device of item 4479 wherein the agent causes DNA damage.

4528. The device of item 4479 wherein the agent is a DNA intercalation agent.

4529. The device of item 4479 wherein the agent is a RNA synthesis inhibitor.

4530. The device of item 4479 wherein the agent is a pyrimidine synthesis inhibitor.

4531. The device of item 4479 wherein the agent inhibits ribonucleotide synthesis or function.

4532. The device of item 4479 wherein the agent inhibits thymidine monophosphate synthesis or function.

4533. The device of item 4479 wherein the agent inhibits DNA synthesis.

4534. The device of item 4479 wherein the agent causes DNA adduct formation.

4535. The device of item 4479 wherein the agent inhibits protein synthesis.

4536. The device of item 4479 wherein the agent inhibits microtubule function.

4537. The device of item 4479 wherein the agent is a cyclin dependent protein kinase inhibitor.

4538. The device of item 4479 wherein the agent is an epidermal growth factor kinase inhibitor.

4539. The device of item 4479 wherein the agent is an elastase inhibitor.

4540. The device of item 4479 wherein the agent is a factor Xa inhibitor.

4541. The device of item 4479 wherein the agent is a farnesyltransferase inhibitor.

4542. The device of item 4479 wherein the agent is a fibrinogen antagonist.

4543. The device of item 4479 wherein the agent is a guanylate cyclase stimulant.

4544. The device of item 4479 wherein the agent is a heat shock protein 90 antagonist.

4545. The device of item 4479 wherein the agent is a heat shock protein 90 antagonist, wherein the heat shock protein 90 antagonist is geldanamycin or an analogue or derivative thereof.

4546. The device of item 4479 wherein the agent is a guanylate cyclase stimulant.

4547. The device of item 4479 wherein the agent is a HMGCoA reductase inhibitor.

4548. The device of item 4479 wherein the agent is a HMGCoA reductase inhibitor, wherein the HMGCoA reductase inhibitor is simvastatin or an analogue or derivative thereof.

4549. The device of item 4479 wherein the agent is a hydroorotate dehydrogenase inhibitor.

4550. The device of item 4479 wherein the agent is an IKK2 inhibitor.

4551. The device of item 4479 wherein the agent is an IL-1 antagonist.

4552. The device of item 4479 wherein the agent is an ICE antagonist.

4553. The device of item 4479 wherein the agent is an IRAK antagonist.

4554. The device of item 4479 wherein the agent is an IL-4 agonist.

4555. The device of item 4479 wherein the agent is an immunomodulatory agent.

4556. The device of item 4479 wherein the agent is sirolimus or an analogue or derivative thereof.

4557. The device of item 4479 wherein the agent is not sirolimus.

4558. The device of item 4479 wherein the agent is everolimus or an analogue or derivative thereof.

4559. The device of item 4479 wherein the agent is tacrolimus or an analogue or derivative thereof.

4560. The device of item 4479 wherein the agent is not tacrolimus.

4561. The device of item 4479 wherein the agent is biolmus or an analogue or derivative thereof.

4562. The device of item 4479 wherein the agent is tresperimus or an analogue or derivative thereof.

4563. The device of item 4479 wherein the agent is auranofin or an analogue or derivative thereof.

4564. The device of item 4479 wherein the agent is 27-0-demethylrapamycin or an analogue or derivative thereof.

4565. The device of item 4479 wherein the agent is gusperimus or an analogue or derivative thereof.

4566. The device of item 4479 wherein the agent is pimecrolimus or an analogue or derivative thereof.

4567. The device of item 4479 wherein the agent is ABT-578 or an analogue or derivative thereof.

4568. The device of item 4479 wherein the agent is an inosine monophosphate dehydrogenase (IMPDH) inhibitor.

4569. The device of item 4479 wherein the agent is an IMPDH inhibitor, wherein the IMPDH inhibitor is mycophenolic acid or an analogue or derivative thereof.

4570. The device of item 4479 wherein the agent is an IMPDH inhibitor, wherein the IMPDH inhibitor is 1-alpha-25 dihydroxy vitamin D3 or an analogue or derivative thereof.

4571. The device of item 4479 wherein the agent is a leukotriene inhibitor.

4572. The device of item 4479 wherein the agent is a MCP-1 antagonist.

4573. The device of item 4479 wherein the agent is a MMP inhibitor.

4574. The device of item 4479 wherein the agent is an NF kappa B inhibitor.

4575. The device of item 4479 wherein the agent is an NF kappa B inhibitor, wherein the NF kappa B inhibitor is Bay 11-7082.

4576. The device of item 4479 wherein the agent is an NO agonist.

4577. The device of item 4479 wherein the agent is a p38 MAP kinase inhibitor.

4578. The device of item 4479 wherein the agent is a p38 MAP kinase inhibitor, wherein the p38 MAP kinase inhibitor is SB 202190.

4579. The device of item 4479 wherein the agent is a phosphodiesterase inhibitor.

4580. The device of item 4479 wherein the agent is a TGF beta inhibitor.

4581. The device of item 4479 wherein the agent is a thromboxane A2 antagonist.

4582. The device of item 4479 wherein the agent is a TNFa antagonist.

4583. The device of item 4479 wherein the agent is a TACE inhibitor.

4584. The device of item 4479 wherein the agent is a tyrosine kinase inhibitor.

4585. The device of item 4479 wherein the agent is a vitronectin inhibitor.

4586. The device of item 4479 wherein the agent is a fibroblast growth factor inhibitor.

4587. The device of item 4479 wherein the agent is a protein kinase inhibitor.

4588. The device of item 4479 wherein the agent is a PDGF receptor kinase inhibitor.

4589. The device of item 4479 wherein the agent is an endothelial growth factor receptor kinase inhibitor.

4590. The device of item 4479 wherein the agent is a retinoic acid receptor antagonist.

4591. The device of item 4479 wherein the agent is a platelet derived growth factor receptor kinase inhibitor.

4592. The device of item 4479 wherein the agent is a fibronogin antagonist.

4593. The device of item 4479 wherein the agent is an antimycotic agent.

4594. The device of item 4479 wherein the agent is an antimycotic agent, wherein the antimycotic agent is sulconizole.

4595. The device of item 4479 wherein the agent is a bisphosphonate.

4596. The device of item 4479 wherein the agent is a phospholipase A1 inhibitor.

4597. The device of item 4479 wherein the agent is a histamine H1/H2/H3 receptor antagonist.

4598. The device of item 4479 wherein the agent is a macrolide antibiotic.

4599. The device of item 4479 wherein the agent is a GPIIb/IIIa receptor antagonist.

4600. The device of item 4479 wherein the agent is an endothelin receptor antagonist.

4601. The device of item 4479 wherein the agent is a peroxisome proliferator-activated receptor agonist.

4602. The device of item 4479 wherein the agent is an estrogen receptor agent.

4603. The device of item 4479 wherein the agent is a somastostatin analogue.

4604. The device of item 4479 wherein the agent is a neurokinin 1 antagonist.

4605. The device of item 4479 wherein the agent is a neurokinin 3 antagonist.

4606. The device of item 4479 wherein the agent is a VLA-4 antagonist.

4607. The device of item 4479 wherein the agent is an osteoclast inhibitor.

4608. The device of item 4479 wherein the agent is a DNA topoisomerase ATP hydrolyzing inhibitor.

4609. The device of item 4479 wherein the agent is an angiotensin I converting enzyme inhibitor.

4610. The device of item 4479 wherein the agent is an angiotensin II antagonist.

4611. The device of item 4479 wherein the agent is an enkephalinase inhibitor.

4612. The device of item 4479 wherein the agent is a peroxisome proliferator-activated receptor gamma agonist insulin sensitizer.

4613. The device of item 4479 wherein the agent is a protein kinase C inhibitor.

4614. The device of item 4479 wherein the agent is a ROCK (rho-associated kinase) inhibitor.

4615. The device of item 4479 wherein the agent is a CXCR3 inhibitor.

4616. The device of item 4479 wherein the agent is an Itk inhibitor.

4617. The device of item 4479 wherein the agent is a cytosolic phospholipase A2-alpha inhibitor.

4618. The device of item 4479 wherein the agent is a PPAR agonist.

4619. The device of item 4479 wherein the agent is an immunosuppressant.

4620. The device of item 4479 wherein the agent is an Erb inhibitor.

4621. The device of item 4479 wherein the agent is an apoptosis agonist.

4622. The device of item 4479 wherein the agent is a lipocortin agonist.

4623. The device of item 4479 wherein the agent is a VCAM-1 antagonist.

4624. The device of item 4479 wherein the agent is a collagen antagonist.

4625. The device of item 4479 wherein the agent is an alpha 2 integrin antagonist.

4626. The device of item 4479 wherein the agent is a TNF alpha inhibitor.

4627. The device of item 4479 wherein the agent is a nitric oxide inhibitor.

4628. The device of item 4479 wherein the agent is a cathepsin inhibitor.

4629. The device of item 4479 wherein the agent is not an anti-inflammatory agent.

4630. The device of item 4479 wherein the agent is not a steroid.

4631. The device of item 4479 wherein the agent is not a glucocorticosteroid.

4632. The device of item 4479 wherein the agent is not dexamethasone.

4633. The device of item 4479 wherein the agent is not an anti-infective agent.

4634. The device of item 4479 wherein the agent is not an antibiotic.

4635. The device of item 4479 wherein the agent is not an anti-fungal agent.

4636. The device of item 4479, further comprising a polymer.

4637. The device of item 4479, further comprising a polymeric carrier.

4638. The device of item 4479 wherein the anti-scarring agent inhibits adhesion between the device and a host into which the device is implanted.

4639. The device of item 4479 wherein the device delivers the anti-scarring agent locally to tissue proximate to the device.

4640. The device of item 4479, further comprising a coating, wherein the coating comprises the anti-scarring agent.

4641. The device of item 4479, further comprising a coating, wherein the coating is disposed on a surface of the device.

4642. The device of item 4479, further comprising a coating, wherein the coating directly contacts the device.

4643. The device of item 4479, further comprising a coating, wherein the coating indirectly contacts the device.

4644. The device of item 4479, further comprising a coating, wherein the coating partially covers the device.

4645. The device of item 4479, further comprising a coating, wherein the coating completely covers the device.

4646. The device of item 4479, further comprising a coating, wherein the coating is a uniform coating.

4647. The device of item 4479, further comprising a coating, wherein the coating is a non-uniform coating.

4648. The device of item 4479, further comprising a coating, wherein the coating is a discontinuous coating.

4649. The device of item 4479, further comprising a coating, wherein the coating is a patterned coating.

4650. The device of item 4479, further comprising a coating, wherein the coating has a thickness of 100 μm or less.

4651. The device of item 4479, further comprising a coating, wherein the coating has a thickness of 10 μm or less.

4652. The device of item 4479, further comprising a coating, wherein the coating adheres to the surface of the device upon deployment of the device.

4653. The device of item 4479, further comprising a coating, wherein the coating is stable at room temperature for a period of 1 year.

4654. The device of item 4479, further comprising a coating, wherein the anti-scarring agent is present in the coating in an amount ranging between about 0.0001% to about 1% by weight.

4655. The device of item 4479, further comprising a coating, wherein the anti-scarring agent is present in the coating in an amount ranging between about 1% to about 10% by weight.

4656. The device of item 4479, further comprising a coating, wherein the anti-scarring agent is present in the coating in an amount ranging between about 10% to about 25% by weight.

4657. The device of item 4479, further comprising a coating, wherein the anti-scarring agent is present in the coating in an amount ranging between about 25% to about 70% by weight.

4658. The device of item 4479, further comprising a coating, wherein the coating further comprises a polymer.

4659. The device of item 4479, further comprising a first coating having a first composition and the second coating having a second composition.

4660. The device of item 4479, further comprising a first coating having a first composition and the second coating having a second composition, wherein the first composition and the second composition are different.

4661. The device of item 4479, further comprising a polymer.

4662. The device of item 4479, further comprising a polymeric carrier.

4663. The device of item 4479, further comprising a polymeric carrier, wherein the polymeric carrier comprises a copolymer.

4664. The device of item 4479, further comprising a polymeric carrier, wherein the polymeric carrier comprises a block copolymer.

4665. The device of item 4479, further comprising a polymeric carrier, wherein the polymeric carrier comprises a random copolymer.

4666. The device of item 4479, further comprising a polymeric carrier, wherein the polymeric carrier comprises a biodegradable polymer.

4667. The device of item 4479, further comprising a polymeric carrier, wherein the polymeric carrier comprises a non-biodegradable polymer.

4668. The device of item 4479, further comprising a polymeric carrier, wherein the polymeric carrier comprises a hydrophilic polymer.

4669. The device of item 4479, further comprising a polymeric carrier, wherein the polymeric carrier comprises a hydrophobic polymer.

4670. The device of item 4479, further comprising a polymeric carrier, wherein the polymeric carrier comprises a polymer having hydrophilic domains.

4671. The device of item 4479, further comprising a polymeric carrier, wherein the polymeric carrier comprises a polymer having hydrophobic domains.

4672. The device of item 4479, further comprising a polymeric carrier, wherein the polymeric carrier comprises a non-conductive polymer.

4673. The device of item 4479, further comprising a polymeric carrier, wherein the polymeric carrier comprises an elastomer.

4674. The device of item 4479, further comprising a polymeric carrier, wherein the polymeric carrier comprises a hydrogel.

4675. The device of item 4479, further comprising a polymeric carrier, wherein the polymeric carrier comprises a silicone polymer.

4676. The device of item 4479, further comprising a polymeric carrier, wherein the polymeric carrier comprises a hydrocarbon polymer.

4677. The device of item 4479, further comprising a polymeric carrier, wherein the polymeric carrier comprises a styrene-derived polymer.

4678. The device of item 4479, further comprising a polymeric carrier, wherein the polymeric carrier comprises a butadiene polymer.

4679. The device of item 4479, further comprising a polymeric carrier, wherein the polymeric carrier comprises a macromer.

4680. The device of item 4479, further comprising a polymeric carrier, wherein the polymeric carrier comprises a poly(ethylene glycol)polymer.

4681. The device of item 4479, further comprising a polymeric carrier, wherein the polymeric carrier comprises an amorphous polymer.

4682. The device of item 4479, further comprising a lubricious coating.

4683. The device of item 4479 wherein the anti-scarring agent is located within pores or holes of the device.

4684. The device of item 4479 wherein the anti-scarring agent is located within a channel, lumen, or divet of the device.

4685. The device of item 4479, further comprising a second pharmaceutically active agent.

4686. The device of item 4479, further comprising an anti-inflammatory agent.

4687. The device of item 4479, further comprising an agent that inhibits infection.

4688. The device of item 4479, further comprising an agent that inhibits infection, wherein the agent is an anthracycline.

4689. The device of item 4479, further comprising an agent that inhibits infection, wherein the agent is doxorubicin.

4690. The device of item 4479, further comprising an agent that inhibits infection, wherein the agent is mitoxantrone.

4691. The device of item 4479, further comprising an agent that inhibits infection, wherein the agent is a fluoropyrimidine.

4692. The device of item 4479, further comprising an agent that inhibits infection, wherein the agent is 5-fluorouracil (5-FU).

4693. The device of item 4479, further comprising an agent that inhibits infection, wherein the agent is a folic acid antagonist.

4694. The device of item 4479, further comprising an agent that inhibits infection, wherein the agent is methotrexate.

4695. The device of item 4479, further comprising an agent that inhibits infection, wherein the agent is a podophylotoxin.

4696. The device of item 4479, further comprising an agent that inhibits infection, wherein the agent is etoposide.

4697. The device of item 4479, further comprising an agent that inhibits infection, wherein the agent is a camptothecin.

4698. The device of item 4479, further comprising an agent that inhibits infection, wherein the agent is a hydroxyurea.

4699. The device of item 4479, further comprising an agent that inhibits infection, wherein the agent is a platinum complex.

4700. The device of item 4479, further comprising an agent that inhibits infection, wherein the agent is cisplatin.

4701. The device of item 4479, further comprising an anti-thrombotic agent.

4702. The device of item 4479, further comprising a visualization agent.

4703. The device of item 4479, further comprising a visualization agent, wherein the visualization agent is a radiopaque material, wherein the radiopaque material comprises a metal, a halogenated compound, or a barium containing compound.

4704. The device of item 4479, further comprising a visualization agent, wherein the visualization agent is a radiopaque material, wherein the radiopaque material comprises barium, tantalum, or technetium.

4705. The device of item 4479, further comprising a visualization agent, wherein the visualization agent is a MRI responsive material.

4706. The device of item 4479, further comprising a visualization agent, wherein the visualization agent comprises a gadolinium chelate.

4707. The device of item 4479, further comprising a visualization agent, wherein the visualization agent comprises iron, magnesium, manganese, copper, or chromium.

4708. The device of item 4479, further comprising a visualization agent, wherein the visualization agent comprises an iron oxide compound.

4709. The device of item 4479, further comprising a visualization agent, wherein the visualization agent comprises a dye, pigment, or colorant.

4710. The device of item 4479, further comprising an echogenic material.

4711. The device of item 4479, further comprising an echogenic material, wherein the echogenic material is in the form of a coating.

4712. The device of item 4479 wherein the device is sterile.

4713. The device of item 4479 wherein the anti-scarring agent is released into tissue in the vicinity of the device after deployment of the device.

4714. The device of item 4479 wherein the anti-scarring agent is released into tissue in the vicinity of the device after deployment of the device, wherein the tissue is connective tissue.

4715. The device of item 4479 wherein the anti-scarring agent is released into tissue in the vicinity of the device after deployment of the device, wherein the tissue is muscle tissue.

4716. The device of item 4479 wherein the anti-scarring agent is released into tissue in the vicinity of the device after deployment of the device, wherein the tissue is nerve tissue.

4717. The device of item 4479 wherein the anti-scarring agent is released into tissue in the vicinity of the device after deployment of the device, wherein the tissue is epithelium tissue.

4718. The device of item 4479 wherein the anti-scarring agent is released in effective concentrations from the device over a period ranging from the time of deployment of the device to about 1 year.

4719. The device of item 4479 wherein the anti-scarring agent is released in effective concentrations from the device over a period ranging from about 1 month to 6 months.

4720. The device of item 4479 wherein the anti-scarring agent is released in effective concentrations from the device over a period ranging from about 1-90 days.

4721. The device of item 4479 wherein the anti-scarring agent is released in effective concentrations from the device at a constant rate.

4722. The device of item 4479 wherein the anti-scarring agent is released in effective concentrations from the device at an increasing rate.

4723. The device of item 4479 wherein the anti-scarring agent is released in effective concentrations from the device at a decreasing rate.

4724. The device of item 4479 wherein the anti-scarring agent is released in effective concentrations from the composition comprising the anti-scarring agent by diffusion over a period ranging from the time of deployment of the device to about 90 days.

4725. The device of item 4479 wherein the anti-scarring agent is released in effective concentrations from the composition comprising the anti-scarring agent by erosion of the composition over a period ranging from the time of deployment of the device to about 90 days.

4726. The device of item 4479 wherein the device comprises about 0.01 μg to about 10 μg of the anti-scarring agent.

4727. The device of item 4479 wherein the device comprises about 10 μg to about 10 mg of the anti-scarring agent.

4728. The device of item 4479 wherein the device comprises about 10 mg to about 250 mg of the anti-scarring agent.

4729. The device of item 4479 wherein the device comprises about 250 mg to about 1000 mg of the anti-scarring agent.

4730. The device of item 4479 wherein the device comprises about 1000 mg to about 2500 mg of the anti-scarring agent.

4731. The device of item 4479 wherein a surface of the device comprises less than 0.01 μg of the anti-scarring agent per mm2 of device surface to which the anti-scarring agent is applied.

4732. The device of item 4479 wherein a surface of the device comprises about 0.01 μg to about 1 μg of the anti-scarring agent per mm2 of device surface to which the anti-scarring agent is applied.

4733. The device of item 4479 wherein a surface of the device comprises about 1 μg to about 10 μg of the anti-scarring agent per mm2 of device surface to which the anti-scarring agent is applied.

4734. The device of item 4479 wherein a surface of the device comprises about 10 μg to about 250 μg of the anti-scarring agent per mm2 of device surface to which the anti-scarring agent is applied.

4735. The device of item 4479 wherein a surface of the device comprises about 250 μg to about 1000 μg of the anti-scarring agent of anti-scarring agent per mm2 of device surface to which the anti-scarring agent is applied.

4736. The device of item 4479 wherein a surface of the device comprises about 1000 μg to about 2500 μg of the anti-scarring agent per mm2 of device surface to which the anti-scarring agent is applied.

4737. A device, comprising an implant that provides a surgical adhesion barrier and an anti-scarring agent or a composition comprising an anti-scarring agent, wherein the agent inhibits scarring between the device and a host into which the device is implanted.

4738. The device of item 4737 wherein the agent inhibits cell regeneration.

4739. The device of item 4737 wherein the agent inhibits angiogenesis.

4740. The device of item 4737 wherein the agent inhibits fibroblast migration.

4741. The device of item 4737 wherein the agent inhibits fibroblast proliferation.

4742. The device of item 4737 wherein the agent inhibits deposition of extracellular matrix.

4743. The device of item 4737 wherein the agent inhibits tissue remodeling.

4744. The device of item 4737 wherein the agent is an angiogenesis inhibitor.

4745. The device of item 4737 wherein the agent is a 5-lipoxygenase inhibitor or antagonist.

4746. The device of item 4737 wherein the agent is a chemokine receptor antagonist.

4747. The device of item 4737 wherein the agent is a cell cycle inhibitor.

4748. The device of item 4737 wherein the agent is a taxane.

4749. The device of item 4737 wherein the agent is an anti-microtubule agent.

4750. The device of item 4737 wherein the agent is paclitaxel.

4751. The device of item 4737 wherein the agent is not paclitaxel.

4752. The device of item 4737 wherein the agent is an analogue or derivative of paclitaxel.

4753. The device of item 4737 wherein the agent is a vinca alkaloid.

4754. The device of item 4737 wherein the agent is camptothecin or an analogue or derivative thereof.

4755. The device of item 4737 wherein the agent is a podophyllotoxin.

4756. The device of item 4737 wherein the agent is a podophyllotoxin, wherein the podophyllotoxin is etoposide or an analogue or derivative thereof.

4757. The device of item 4737 wherein the agent is an anthracycline.

4758. The device of item 4737 wherein the agent is an anthracycline, wherein the anthracycline is doxorubicin or an analogue or derivative thereof.

4759. The device of item 4737 wherein the agent is an anthracycline, wherein the anthracycline is mitoxantrone or an analogue or derivative thereof.

4760. The device of item 4737 wherein the agent is a platinum compound.

4761. The device of item 4737 wherein the agent is a nitrosourea.

4762. The device of item 4737 wherein the agent is a nitroimidazole.

4763. The device of item 4737 wherein the agent is a folic acid antagonist.

4764. The device of item 4737 wherein the agent is a cytidine analogue.

4765. The device of item 4737 wherein the agent is a pyrimidine analogue.

4766. The device of item 4737 wherein the agent is a fluoropyrimidine analogue.

4767. The device of item 4737 wherein the agent is a purine analogue.

4768. The device of item 4737 wherein the agent is a nitrogen mustard or an analogue or derivative thereof.

4769. The device of item 4737 wherein the agent is a hydroxyurea.

4770. The device of item 4737 wherein the agent is a mytomicin or an analogue or derivative thereof.

4771. The device of item 4737 wherein the agent is an alkyl sulfonate.

4772. The device of item 4737 wherein the agent is a benzamide or an analogue or derivative thereof.

4773. The device of item 4737 wherein the agent is a nicotinamide or an analogue or derivative thereof.

4774. The device of item 4737 wherein the agent is a halogenated sugar or an analogue or derivative thereof.

4775. The device of item 4737 wherein the agent is a DNA alkylating agent.

4776. The device of item 4737 wherein the agent is an anti-microtubule agent.

4777. The device of item 4737 wherein the agent is a topoisomerase inhibitor.

4778. The device of item 4737 wherein the agent is a DNA cleaving agent.

4779. The device of item 4737 wherein the agent is an antimetabolite.

4780. The device of item 4737 wherein the agent inhibits adenosine deaminase.

4781. The device of item 4737 wherein the agent inhibits purine ring synthesis.

4782. The device of item 4737 wherein the agent is a nucleotide interconversion inhibitor.

4783. The device of item 4737 wherein the agent inhibits dihydrofolate reduction.

4784. The device of item 4737 wherein the agent blocks thymidine monophosphate.

4785. The device of item 4737 wherein the agent causes DNA damage.

4786. The device of item 4737 wherein the agent is a DNA intercalation agent.

4787. The device of item 4737 wherein the agent is a RNA synthesis inhibitor.

4788. The device of item 4737 wherein the agent is a pyrimidine synthesis inhibitor.

4789. The device of item 4737 wherein the agent inhibits ribonucleotide synthesis or function.

4790. The device of item 4737 wherein the agent inhibits thymidine monophosphate synthesis or function.

4791. The device of item 4737 wherein the agent inhibits DNA synthesis.

4792. The device of item 4737 wherein the agent causes DNA adduct formation.

4793. The device of item 4737 wherein the agent inhibits protein synthesis.

4794. The device of item 4737 wherein the agent inhibits microtubule function.

4795. The device of item 4737 wherein the agent is a cyclin dependent protein kinase inhibitor.

4796. The device of item 4737 wherein the agent is an epidermal growth factor kinase inhibitor.

4797. The device of item 4737 wherein the agent is an elastase inhibitor.

4798. The device of item 4737 wherein the agent is a factor Xa inhibitor.

4799. The device of item 4737 wherein the agent is a farnesyltransferase inhibitor.

4800. The device of item 4737 wherein the agent is a fibrinogen antagonist.

4801. The device of item 4737 wherein the agent is a guanylate cyclase stimulant.

4802. The device of item 4737 wherein the agent is a heat shock protein 90 antagonist.

4803. The device of item 4737 wherein the agent is a heat shock protein 90 antagonist, wherein the heat shock protein 90 antagonist is geldanamycin or an analogue or derivative thereof.

4804. The device of item 4737 wherein the agent is a guanylate cyclase stimulant.

4805. The device of item 4737 wherein the agent is a HMGCoA reductase inhibitor.

4806. The device of item 4737 wherein the agent is a HMGCoA reductase inhibitor, wherein the HMGCoA reductase inhibitor is simvastatin or an analogue or derivative thereof.

4807. The device of item 4737 wherein the agent is a hydroorotate dehydrogenase inhibitor.

4808. The device of item 4737 wherein the agent is an IKK2 inhibitor.

4809. The device of item 4737 wherein the agent is an IL-1 antagonist.

4810. The device of item 4737 wherein the agent is an ICE antagonist.

4811. The device of item 4737 wherein the agent is an IRAK antagonist.

4812. The device of item 4737 wherein the agent is an IL-4 agonist.

4813. The device of item 4737 wherein the agent is an immunomodulatory agent.

4814. The device of item 4737 wherein the agent is sirolimus or an analogue or derivative thereof.

4815. The device of item 4737 wherein the agent is not sirolimus.

4816. The device of item 4737 wherein the agent is everolimus or an analogue or derivative thereof.

4817. The device of item 4737 wherein the agent is tacrolimus or an analogue or derivative thereof.

4818. The device of item 4737 wherein the agent is not tacrolimus.

4819. The device of item 4737 wherein the agent is biolmus or an analogue or derivative thereof.

4820. The device of item 4737 wherein the agent is tresperimus or an analogue or derivative thereof.

4821. The device of item 4737 wherein the agent is auranofin or an analogue or derivative thereof.

4822. The device of item 4737 wherein the agent is 27-0-demethylrapamycin or an analogue or derivative thereof.

4823. The device of item 4737 wherein the agent is gusperimus or an analogue or derivative thereof.

4824. The device of item 4737 wherein the agent is pimecrolimus or an analogue or derivative thereof.

4825. The device of item 4737 wherein the agent is ABT-578 or an analogue or derivative thereof.

4826. The device of item 4737 wherein the agent is an inosine monophosphate dehydrogenase (IMPDH) inhibitor.

4827. The device of item 4737 wherein the agent is an IMPDH inhibitor, wherein the IMPDH inhibitor is mycophenolic acid or an analogue or derivative thereof.

4828. The device of item 4737 wherein the agent is an IMPDH inhibitor, wherein the IMPDH inhibitor is 1-alpha-25 dihydroxy vitamin D3 or an analogue or derivative thereof.

4829. The device of item 4737 wherein the agent is a leukotriene inhibitor.

4830. The device of item 4737 wherein the agent is a MCP-1 antagonist.

4831. The device of item 4737 wherein the agent is a MMP inhibitor.

4832. The device of item 4737 wherein the agent is an NF kappa B inhibitor.

4833. The device of item 4737 wherein the agent is an NF kappa B inhibitor, wherein the NF kappa B inhibitor is Bay 11-7082.

4834. The device of item 4737 wherein the agent is an NO agonist.

4835. The device of item 4737 wherein the agent is a p38 MAP kinase inhibitor.

4836. The device of item 4737 wherein the agent is a p38 MAP kinase inhibitor, wherein the p38 MAP kinase inhibitor is SB 202190.

4837. The device of item 4737 wherein the agent is a phosphodiesterase inhibitor.

4838. The device of item 4737 wherein the agent is a TGF beta inhibitor.

4839. The device of item 4737 wherein the agent is a thromboxane A2 antagonist.

4840. The device of item 4737 wherein the agent is a TNFa antagonist.

4841. The device of item 4737 wherein the agent is a TACE inhibitor.

4842. The device of item 4737 wherein the agent is a tyrosine kinase inhibitor.

4843. The device of item 4737 wherein the agent is a vitronectin inhibitor.

4844. The device of item 4737 wherein the agent is a fibroblast growth factor inhibitor.

4845. The device of item 4737 wherein the agent is a protein kinase inhibitor.

4846. The device of item 4737 wherein the agent is a PDGF receptor kinase inhibitor.

4847. The device of item 4737 wherein the agent is an endothelial growth factor receptor kinase inhibitor.

4848. The device of item 4737 wherein the agent is a retinoic acid receptor antagonist.

4849. The device of item 4737 wherein the agent is a platelet derived growth factor receptor kinase inhibitor.

4850. The device of item 4737 wherein the agent is a fibronogin antagonist.

4851. The device of item 4737 wherein the agent is an antimycotic agent.

4852. The device of item 4737 wherein the agent is an antimycotic agent, wherein the antimycotic agent is sulconizole.

4853. The device of item 4737 wherein the agent is a bisphosphonate.

4854. The device of item 4737 wherein the agent is a phospholipase A1 inhibitor.

4855. The device of item 4737 wherein the agent is a histamine H1/H2/H3 receptor antagonist.

4856. The device of item 4737 wherein the agent is a macrolide antibiotic.

4857. The device of item 4737 wherein the agent is a GPIIb/IIIa receptor antagonist.

4858. The device of item 4737 wherein the agent is an endothelin receptor antagonist.

4859. The device of item 4737 wherein the agent is a peroxisome proliferator-activated receptor agonist.

4860. The device of item 4737 wherein the agent is an estrogen receptor agent.

4861. The device of item 4737 wherein the agent is a somastostatin analogue.

4862. The device of item 4737 wherein the agent is a neurokinin 1 antagonist.

4863. The device of item 4737 wherein the agent is a neurokinin 3 antagonist.

4864. The device of item 4737 wherein the agent is a VLA-4 antagonist.

4865. The device of item 4737 wherein the agent is an osteoclast inhibitor.

4866. The device of item 4737 wherein the agent is a DNA topoisomerase ATP hydrolyzing inhibitor.

4867. The device of item 4737 wherein the agent is an angiotensin I converting enzyme inhibitor.

4868. The device of item 4737 wherein the agent is an angiotensin II antagonist.

4869. The device of item 4737 wherein the agent is an enkephalinase inhibitor.

4870. The device of item 4737 wherein the agent is a peroxisome proliferator-activated receptor gamma agonist insulin sensitizer.

4871. The device of item 4737 wherein the agent is a protein kinase C inhibitor.

4872. The device of item 4737 wherein the agent is a ROCK (rho-associated kinase) inhibitor.

4873. The device of item 4737 wherein the agent is a CXCR3 inhibitor.

4874. The device of item 4737 wherein the agent is an Itk inhibitor.

4875. The device of item 4737 wherein the agent is a cytosolic phospholipase A2-alpha inhibitor.

4876. The device of item 4737 wherein the agent is a PPAR agonist.

4877. The device of item 4737 wherein the agent is an immunosuppressant.

4878. The device of item 4737 wherein the agent is an Erb inhibitor.

4879. The device of item 4737 wherein the agent is an apoptosis agonist.

4880. The device of item 4737 wherein the agent is a lipocortin agonist.

4881. The device of item 4737 wherein the agent is a VCAM-1 antagonist.

4882. The device of item 4737 wherein the agent is a collagen antagonist.

4883. The device of item 4737 wherein the agent is an alpha 2 integrin antagonist.

4884. The device of item 4737 wherein the agent is a TNF alpha inhibitor.

4885. The device of item 4737 wherein the agent is a nitric oxide inhibitor.

4886. The device of item 4737 wherein the agent is a cathepsin inhibitor.

4887. The device of item 4737 wherein the agent is not an anti-inflammatory agent.

4888. The device of item 4737 wherein the agent is not a steroid.

4889. The device of item 4737 wherein the agent is not a glucocorticosteroid.

4890. The device of item 4737 wherein the agent is not dexamethasone.

4891. The device of item 4737 wherein the agent is not an anti-infective agent.

4892. The device of item 4737 wherein the agent is not an antibiotic.

4893. The device of item 4737 wherein the agent is not an anti-fungal agent.

4894. The device of item 4737, further comprising a polymer.

4895. The device of item 4737, further comprising a polymeric carrier.

4896. The device of item 4737 wherein the anti-scarring agent inhibits adhesion between the device and a host into which the device is implanted.

4897. The device of item 4737 wherein the device delivers the anti-scarring agent locally to tissue proximate to the device.

4898. The device of item 4737, further comprising a coating, wherein the coating comprises the anti-scarring agent.

4899. The device of item 4737, further comprising a coating, wherein the coating is disposed on a surface of the device.

4900. The device of item 4737, further comprising a coating, wherein the coating directly contacts the device.

4901. The device of item 4737, further comprising a coating, wherein the coating indirectly contacts the device.

4902. The device of item 4737, further comprising a coating, wherein the coating partially covers the device.

4903. The device of item 4737, further comprising a coating, wherein the coating completely covers the device.

4904. The device of item 4737, further comprising a coating, wherein the coating is a uniform coating.

4905. The device of item 4737, further comprising a coating, wherein the coating is a non-uniform coating.

4906. The device of item 4737, further comprising a coating, wherein the coating is a discontinuous coating.

4907. The device of item 4737, further comprising a coating, wherein the coating is a patterned coating.

4908. The device of item 4737, further comprising a coating, wherein the coating has a thickness of 100 μm or less.

4909. The device of item 4737, further comprising a coating, wherein the coating has a thickness of 10 μm or less.

4910. The device of item 4737, further comprising a coating, wherein the coating adheres to the surface of the device upon deployment of the device.

4911. The device of item 4737, further comprising a coating, wherein the coating is stable at room temperature for a period of 1 year.

4912. The device of item 4737, further comprising a coating, wherein the anti-scarring agent is present in the coating in an amount ranging between about 0.0001% to about 1% by weight.

4913. The device of item 4737, further comprising a coating, wherein the anti-scarring agent is present in the coating in an amount ranging between about 1% to about 10% by weight.

4914. The device of item 4737, further comprising a coating, wherein the anti-scarring agent is present in the coating in an amount ranging between about 10% to about 25% by weight.

4915. The device of item 4737, further comprising a coating, wherein the anti-scarring agent is present in the coating in an amount ranging between about 25% to about 70% by weight.

4916. The device of item 4737, further comprising a coating, wherein the coating further comprises a polymer.

4917. The device of item 4737, further comprising a first coating having a first composition and the second coating having a second composition.

4918. The device of item 4737, further comprising a first coating having a first composition and the second coating having a second composition, wherein the first composition and the second composition are different.

4919. The device of item 4737, further comprising a polymer.

4920. The device of item 4737, further comprising a polymeric carrier.

4921. The device of item 4737, further comprising a polymeric carrier, wherein the polymeric carrier comprises a copolymer.

4922. The device of item 4737, further comprising a polymeric carrier, wherein the polymeric carrier comprises a block copolymer.

4923. The device of item 4737, further comprising a polymeric carrier, wherein the polymeric carrier comprises a random copolymer.

4924. The device of item 4737, further comprising a polymeric carrier, wherein the polymeric carrier comprises a biodegradable polymer.

4925. The device of item 4737, further comprising a polymeric carrier, wherein the polymeric carrier comprises a non-biodegradable polymer.

4926. The device of item 4737, further comprising a polymeric carrier, wherein the polymeric carrier comprises a hydrophilic polymer.

4927. The device of item 4737, further comprising a polymeric carrier, wherein the polymeric carrier comprises a hydrophobic polymer.

4928. The device of item 4737, further comprising a polymeric carrier, wherein the polymeric carrier comprises a polymer having hydrophilic domains.

4929. The device of item 4737, further comprising a polymeric carrier, wherein the polymeric carrier comprises a polymer having hydrophobic domains.

4930. The device of item 4737, further comprising a polymeric carrier, wherein the polymeric carrier comprises a non-conductive polymer.

4931. The device of item 4737, further comprising a polymeric carrier, wherein the polymeric carrier comprises an elastomer.

4932. The device of item 4737, further comprising a polymeric carrier, wherein the polymeric carrier comprises a hydrogel.

4933. The device of item 4737, further comprising a polymeric carrier, wherein the polymeric carrier comprises a silicone polymer.

4934. The device of item 4737, further comprising a polymeric carrier, wherein the polymeric carrier comprises a hydrocarbon polymer.

4935. The device of item 4737, further comprising a polymeric carrier, wherein the polymeric carrier comprises a styrene-derived polymer.

4936. The device of item 4737, further comprising a polymeric carrier, wherein the polymeric carrier comprises a butadiene polymer.

4937. The device of item 4737, further comprising a polymeric carrier, wherein the polymeric carrier comprises a macromer.

4938. The device of item 4737, further comprising a polymeric carrier, wherein the polymeric carrier comprises a poly(ethylene glycol)polymer.

4939. The device of item 4737, further comprising a polymeric carrier, wherein the polymeric carrier comprises an amorphous polymer.

4940. The device of item 4737, further comprising a lubricious coating.

4941. The device of item 4737 wherein the anti-scarring agent is located within pores or holes of the device.

4942. The device of item 4737 wherein the anti-scarring agent is located within a channel, lumen, or divet of the device.

4943. The device of item 4737, further comprising a second pharmaceutically active agent.

4944. The device of item 4737, further comprising an anti-inflammatory agent.

4945. The device of item 4737, further comprising an agent that inhibits infection.

4946. The device of item 4737, further comprising an agent that inhibits infection, wherein the agent is an anthracycline.

4947. The device of item 4737, further comprising an agent that inhibits infection, wherein the agent is doxorubicin.

4948. The device of item 4737, further comprising an agent that inhibits infection, wherein the agent is mitoxantrone.

4949. The device of item 4737, further comprising an agent that inhibits infection, wherein the agent is a fluoropyrimidine.

4950. The device of item 4737, further comprising an agent that inhibits infection, wherein the agent is 5-fluorouracil (5-FU).

4951. The device of item 4737, further comprising an agent that inhibits infection, wherein the agent is a folic acid antagonist.

4952. The device of item 4737, further comprising an agent that inhibits infection, wherein the agent is methotrexate.

4953. The device of item 4737, further comprising an agent that inhibits infection, wherein the agent is a podophylotoxin.

4954. The device of item 4737, further comprising an agent that inhibits infection, wherein the agent is etoposide.

4955. The device of item 4737, further comprising an agent that inhibits infection, wherein the agent is a camptothecin.

4956. The device of item 4737, further comprising an agent that inhibits infection, wherein the agent is a hydroxyurea.

4957. The device of item 4737, further comprising an agent that inhibits infection, wherein the agent is a platinum complex.

4958. The device of item 4737, further comprising an agent that inhibits infection, wherein the agent is cisplatin.

4959. The device of item 4737, further comprising an anti-thrombotic agent.

4960. The device of item 4737, further comprising a visualization agent.

4961. The device of item 4737, further comprising a visualization agent, wherein the visualization agent is a radiopaque material, wherein the radiopaque material comprises a metal, a halogenated compound, or a barium containing compound.

4962. The device of item 4737, further comprising a visualization agent, wherein the visualization agent is a radiopaque material, wherein the radiopaque material comprises barium, tantalum, or technetium.

4963. The device of item 4737, further comprising a visualization agent, wherein the visualization agent is a MRI responsive material.

4964. The device of item 4737, further comprising a visualization agent, wherein the visualization agent comprises a gadolinium chelate.

4965. The device of item 4737, further comprising a visualization agent, wherein the visualization agent comprises iron, magnesium, manganese, copper, or chromium.

4966. The device of item 4737, further comprising a visualization agent, wherein the visualization agent comprises an iron oxide compound.

4967. The device of item 4737, further comprising a visualization agent, wherein the visualization agent comprises a dye, pigment, or colorant.

4968. The device of item 4737, further comprising an echogenic material.

4969. The device of item 4737, further comprising an echogenic material, wherein the echogenic material is in the form of a coating.

4970. The device of item 4737 wherein the device is sterile.

4971. The device of item 4737 wherein the anti-scarring agent is released into tissue in the vicinity of the device after deployment of the device.

4972. The device of item 4737 wherein the anti-scarring agent is released into tissue in the vicinity of the device after deployment of the device, wherein the tissue is connective tissue.

4973. The device of item 4737 wherein the anti-scarring agent is released into tissue in the vicinity of the device after deployment of the device, wherein the tissue is muscle tissue.

4974. The device of item 4737 wherein the anti-scarring agent is released into tissue in the vicinity of the device after deployment of the device, wherein the tissue is nerve tissue.

4975. The device of item 4737 wherein the anti-scarring agent is released into tissue in the vicinity of the device after deployment of the device, wherein the tissue is epithelium tissue.

4976. The device of item 4737 wherein the anti-scarring agent is released in effective concentrations from the device over a period ranging from the time of deployment of the device to about 1 year.

4977. The device of item 4737 wherein the anti-scarring agent is released in effective concentrations from the device over a period ranging from about 1 month to 6 months.

4978. The device of item 4737 wherein the anti-scarring agent is released in effective concentrations from the device over a period ranging from about 1-90 days.

4979. The device of item 4737 wherein the anti-scarring agent is released in effective concentrations from the device at a constant rate.

4980. The device of item 4737 wherein the anti-scarring agent is released in effective concentrations from the device at an increasing rate.

4981. The device of item 4737 wherein the anti-scarring agent is released in effective concentrations from the device at a decreasing rate.

4982. The device of item 4737 wherein the anti-scarring agent is released in effective concentrations from the composition comprising the anti-scarring agent by diffusion over a period ranging from the time of deployment of the device to about 90 days.

4983. The device of item 4737 wherein the anti-scarring agent is released in effective concentrations from the composition comprising the anti-scarring agent by erosion of the composition over a period ranging from the time of deployment of the device to about 90 days.

4984. The device of item 4737 wherein the device comprises about 0.01 μg to about 10 μg of the anti-scarring agent.

4985. The device of item 4737 wherein the device comprises about 10 μg to about 10 mg of the anti-scarring agent.

4986. The device of item 4737 wherein the device comprises about 10 mg to about 250 mg of the anti-scarring agent.

4987. The device of item 4737 wherein the device comprises about 250 mg to about 1000 mg of the anti-scarring agent.

4988. The device of item 4737 wherein the device comprises about 1000 mg to about 2500 mg of the anti-scarring agent.

4989. The device of item 4737 wherein a surface of the device comprises less than 0.01 μg of the anti-scarring agent per mm2 of device surface to which the anti-scarring agent is applied.

4990. The device of item 4737 wherein a surface of the device comprises about 0.01 μg to about 1 μg of the anti-scarring agent per mm2 of device surface to which the anti-scarring agent is applied.

4991. The device of item 4737 wherein a surface of the device comprises about 1 μg to about 10 μg of the anti-scarring agent per mm2 of device surface to which the anti-scarring agent is applied.

4992. The device of item 4737 wherein a surface of the device comprises about 10 μg to about 250 μg of the anti-scarring agent per mm2 of device surface to which the anti-scarring agent is applied.

4993. The device of item 4737 wherein a surface of the device comprises about 250 μg to about 1000 μg of the anti-scarring agent of anti-scarring agent per mm2 of device surface to which the anti-scarring agent is applied.

4994. The device of item 4737 wherein a surface of the device comprises about 1000 μg to about 2500 μg of the anti-scarring agent per mm2 of device surface to which the anti-scarring agent is applied.

4995. A device, comprising an implantable nonvascular stent or tube (i.e., an implant) and an anti-scarring agent or a composition comprising an anti-scarring agent, wherein the agent inhibits scarring between the device and a host into which the device is implanted.

4996. The device of item 4995 wherein the agent inhibits cell regeneration.

4997. The device of item 4995 wherein the agent inhibits angiogenesis.

4998. The device of item 4995 wherein the agent inhibits fibroblast migration.

4999. The device of item 4995 wherein the agent inhibits fibroblast proliferation.

5000. The device of item 4995 wherein the agent inhibits deposition of extracellular matrix.

5001. The device of item 4995 wherein the agent inhibits tissue remodeling.

5002. The device of item 4995 wherein the agent is an angiogenesis inhibitor.

5003. The device of item 4995 wherein the agent is a 5-lipoxygenase inhibitor or antagonist.

5004. The device of item 4995 wherein the agent is a chemokine receptor antagonist.

5005. The device of item 4995 wherein the agent is a cell cycle inhibitor.

5006. The device of item 4995 wherein the agent is a taxane.

5007. The device of item 4995 wherein the agent is an anti-microtubule agent.

5008. The device of item 4995 wherein the agent is paclitaxel.

5009. The device of item 4995 wherein the agent is not paclitaxel.

5010. The device of item 4995 wherein the agent is an analogue or derivative of paclitaxel.

5011. The device of item 4995 wherein the agent is a vinca alkaloid.

5012. The device of item 4995 wherein the agent is camptothecin or an analogue or derivative thereof.

5013. The device of item 4995 wherein the agent is a podophyllotoxin.

5014. The device of item 4995 wherein the agent is a podophyllotoxin, wherein the podophyllotoxin is etoposide or an analogue or derivative thereof.

5015. The device of item 4995 wherein the agent is an anthracycline.

5016. The device of item 4995 wherein the agent is an anthracycline, wherein the anthracycline is doxorubicin or an analogue or derivative thereof.

5017. The device of item 4995 wherein the agent is an anthracycline, wherein the anthracycline is mitoxantrone or an analogue or derivative thereof.

5018. The device of item 4995 wherein the agent is a platinum compound.

5019. The device of item 4995 wherein the agent is a nitrosourea.

5020. The device of item 4995 wherein the agent is a nitroimidazole.

5021. The device of item 4995 wherein the agent is a folic acid antagonist.

5022. The device of item 4995 wherein the agent is a cytidine analogue.

5023. The device of item 4995 wherein the agent is a pyrimidine analogue.

5024. The device of item 4995 wherein the agent is a fluoropyrimidine analogue.

5025. The device of item 4995 wherein the agent is a purine analogue.

5026. The device of item 4995 wherein the agent is a nitrogen mustard or an analogue or derivative thereof.

5027. The device of item 4995 wherein the agent is a hydroxyurea.

5028. The device of item 4995 wherein the agent is a mytomicin or an analogue or derivative thereof.

5029. The device of item 4995 wherein the agent is an alkyl sulfonate.

5030. The device of item 4995 wherein the agent is a benzamide or an analogue or derivative thereof.

5031. The device of item 4995 wherein the agent is a nicotinamide or an analogue or derivative thereof.

5032. The device of item 4995 wherein the agent is a halogenated sugar or an analogue or derivative thereof.

5033. The device of item 4995 wherein the agent is a DNA alkylating agent.

5034. The device of item 4995 wherein the agent is an anti-microtubule agent.

5035. The device of item 4995 wherein the agent is a topoisomerase inhibitor.

5036. The device of item 4995 wherein the agent is a DNA cleaving agent.

5037. The device of item 4995 wherein the agent is an antimetabolite.

5038. The device of item 4995 wherein the agent inhibits adenosine deaminase.

5039. The device of item 4995 wherein the agent inhibits purine ring synthesis.

5040. The device of item 4995 wherein the agent is a nucleotide interconversion inhibitor.

5041. The device of item 4995 wherein the agent inhibits dihydrofolate reduction.

5042. The device of item 4995 wherein the agent blocks thymidine monophosphate.

5043. The device of item 4995 wherein the agent causes DNA damage.

5044. The device of item 4995 wherein the agent is a DNA intercalation agent.

5045. The device of item 4995 wherein the agent is a RNA synthesis inhibitor.

5046. The device of item 4995 wherein the agent is a pyrimidine synthesis inhibitor.

5047. The device of item 4995 wherein the agent inhibits ribonucleotide synthesis or function.

5048. The device of item 4995 wherein the agent inhibits thymidine monophosphate synthesis or function.

5049. The device of item 4995 wherein the agent inhibits DNA synthesis.

5050. The device of item 4995 wherein the agent causes DNA adduct formation.

5051. The device of item 4995 wherein the agent inhibits protein synthesis.

5052. The device of item 4995 wherein the agent inhibits microtubule function.

5053. The device of item 4995 wherein the agent is a cyclin dependent protein kinase inhibitor.

5054. The device of item 4995 wherein the agent is an epidermal growth factor kinase inhibitor.

5055. The device of item 4995 wherein the agent is an elastase inhibitor.

5056. The device of item 4995 wherein the agent is a factor Xa inhibitor.

5057. The device of item 4995 wherein the agent is a farnesyltransferase inhibitor.

5058. The device of item 4995 wherein the agent is a fibrinogen antagonist.

5059. The device of item 4995 wherein the agent is a guanylate cyclase stimulant.

5060. The device of item 4995 wherein the agent is a heat shock protein 90 antagonist.

5061. The device of item 4995 wherein the agent is a heat shock protein 90 antagonist, wherein the heat shock protein 90 antagonist is geldanamycin or an analogue or derivative thereof.

5062. The device of item 4995 wherein the agent is a guanylate cyclase stimulant.

5063. The device of item 4995 wherein the agent is a HMGCoA reductase inhibitor.

5064. The device of item 4995 wherein the agent is a HMGCoA reductase inhibitor, wherein the HMGCoA reductase inhibitor is simvastatin or an analogue or derivative thereof.

5065. The device of item 4995 wherein the agent is a hydroorotate dehydrogenase inhibitor.

5066. The device of item 4995 wherein the agent is an IKK2 inhibitor.

5067. The device of item 4995 wherein the agent is an IL-1 antagonist.

5068. The device of item 4995 wherein the agent is an ICE antagonist.

5069. The device of item 4995 wherein the agent is an IRAK antagonist.

5070. The device of item 4995 wherein the agent is an IL-4 agonist.

5071. The device of item 4995 wherein the agent is an immunomodulatory agent.

5072. The device of item 4995 wherein the agent is sirolimus or an analogue or derivative thereof.

5073. The device of item 4995 wherein the agent is not sirolimus.

5074. The device of item 4995 wherein the agent is everolimus or an analogue or derivative thereof.

5075. The device of item 4995 wherein the agent is tacrolimus or an analogue or derivative thereof.

5076. The device of item 4995 wherein the agent is not tacrolimus.

5077. The device of item 4995 wherein the agent is biolmus or an analogue or derivative thereof.

5078. The device of item 4995 wherein the agent is tresperimus or an analogue or derivative thereof.

5079. The device of item 4995 wherein the agent is auranofin or an analogue or derivative thereof.

5080. The device of item 4995 wherein the agent is 27-0-demethylrapamycin or an analogue or derivative thereof.

5081. The device of item 4995 wherein the agent is gusperimus or an analogue or derivative thereof.

5082. The device of item 4995 wherein the agent is pimecrolimus or an analogue or derivative thereof.

5083. The device of item 4995 wherein the agent is ABT-578 or an analogue or derivative thereof.

5084. The device of item 4995 wherein the agent is an inosine monophosphate dehydrogenase (IMPDH) inhibitor.

5085. The device of item 4995 wherein the agent is an IMPDH inhibitor, wherein the IMPDH inhibitor is mycophenolic acid or an analogue or derivative thereof.

5086. The device of item 4995 wherein the agent is an IMPDH inhibitor, wherein the IMPDH inhibitor is 1-alpha-25 dihydroxy vitamin D3 or an analogue or derivative thereof.

5087. The device of item 4995 wherein the agent is a leukotriene inhibitor.

5088. The device of item 4995 wherein the agent is a MCP-1 antagonist.

5089. The device of item 4995 wherein the agent is a MMP inhibitor.

5090. The device of item 4995 wherein the agent is an NF kappa B inhibitor.

5091. The device of item 4995 wherein the agent is an NF kappa B inhibitor, wherein the NF kappa B inhibitor is Bay 11-7082.

5092. The device of item 4995 wherein the agent is an NO agonist.

5093. The device of item 4995 wherein the agent is a p38 MAP kinase inhibitor.

5094. The device of item 4995 wherein the agent is a p38 MAP kinase inhibitor, wherein the p38 MAP kinase inhibitor is SB 202190.

5095. The device of item 4995 wherein the agent is a phosphodiesterase inhibitor.

5096. The device of item 4995 wherein the agent is a TGF beta inhibitor.

5097. The device of item 4995 wherein the agent is a thromboxane A2 antagonist.

5098. The device of item 4995 wherein the agent is a TNFa antagonist.

5099. The device of item 4995 wherein the agent is a TACE inhibitor.

5100. The device of item 4995 wherein the agent is a tyrosine kinase inhibitor.

5101. The device of item 4995 wherein the agent is a vitronectin inhibitor.

5102. The device of item 4995 wherein the agent is a fibroblast growth factor inhibitor.

5103. The device of item 4995 wherein the agent is a protein kinase inhibitor.

5104. The device of item 4995 wherein the agent is a PDGF receptor kinase inhibitor.

5105. The device of item 4995 wherein the agent is an endothelial growth factor receptor kinase inhibitor.

5106. The device of item 4995 wherein the agent is a retinoic acid receptor antagonist.

5107. The device of item 4995 wherein the agent is a platelet derived growth factor receptor kinase inhibitor.

5108. The device of item 4995 wherein the agent is a fibronogin antagonist.

5109. The device of item 4995 wherein the agent is an antimycotic agent.

5110. The device of item 4995 wherein the agent is an antimycotic agent, wherein the antimycotic agent is sulconizole.

5111. The device of item 4995 wherein the agent is a bisphosphonate.

5112. The device of item 4995 wherein the agent is a phospholipase A1 inhibitor.

5113. The device of item 4995 wherein the agent is a histamine H1/H2/H3 receptor antagonist.

5114. The device of item 4995 wherein the agent is a macrolide antibiotic.

5115. The device of item 4995 wherein the agent is a GPIIb/IIIa receptor antagonist.

5116. The device of item 4995 wherein the agent is an endothelin receptor antagonist.

5117. The device of item 4995 wherein the agent is a peroxisome proliferator-activated receptor agonist.

5118. The device of item 4995 wherein the agent is an estrogen receptor agent.

5119. The device of item 4995 wherein the agent is a somastostatin analogue.

5120. The device of item 4995 wherein the agent is a neurokinin 1 antagonist.

5121. The device of item 4995 wherein the agent is a neurokinin 3 antagonist.

5122. The device of item 4995 wherein the agent is a VLA-4 antagonist.

5123. The device of item 4995 wherein the agent is an osteoclast inhibitor.

5124. The device of item 4995 wherein the agent is a DNA topoisomerase ATP hydrolyzing inhibitor.

5125. The device of item 4995 wherein the agent is an angiotensin I converting enzyme inhibitor.

5126. The device of item 4995 wherein the agent is an angiotensin II antagonist.

5127. The device of item 4995 wherein the agent is an enkephalinase inhibitor.

5128. The device of item 4995 wherein the agent is a peroxisome proliferator-activated receptor gamma agonist insulin sensitizer.

5129. The device of item 4995 wherein the agent is a protein kinase C inhibitor.

5130. The device of item 4995 wherein the agent is a ROCK (rho-associated kinase) inhibitor.

5131. The device of item 4995 wherein the agent is a CXCR3 inhibitor.

5132. The device of item 4995 wherein the agent is an Itk inhibitor.

5133. The device of item 4995 wherein the agent is a cytosolic phospholipase A2-alpha inhibitor.

5134. The device of item 4995 wherein the agent is a PPAR agonist.

5135. The device of item 4995 wherein the agent is an immunosuppressant.

5136. The device of item 4995 wherein the agent is an Erb inhibitor.

5137. The device of item 4995 wherein the agent is an apoptosis agonist.

5138. The device of item 4995 wherein the agent is a lipocortin agonist.

5139. The device of item 4995 wherein the agent is a VCAM-1 antagonist.

5140. The device of item 4995 wherein the agent is a collagen antagonist.

5141. The device of item 4995 wherein the agent is an alpha 2 integrin antagonist.

5142. The device of item 4995 wherein the agent is a TNF alpha inhibitor.

5143. The device of item 4995 wherein the agent is a nitric oxide inhibitor.

5144. The device of item 4995 wherein the agent is a cathepsin inhibitor.

5145. The device of item 4995 wherein the agent is not an anti-inflammatory agent.

5146. The device of item 4995 wherein the agent is not a steroid.

5147. The device of item 4995 wherein the agent is not a glucocorticosteroid.

5148. The device of item 4995 wherein the agent is not dexamethasone.

5149. The device of item 4995 wherein the agent is not an anti-infective agent.

5150. The device of item 4995 wherein the agent is not an antibiotic.

5151. The device of item 4995 wherein the agent is not an anti-fungal agent.

5152. The device of item 4995, further comprising a polymer.

5153. The device of item 4995, further comprising a polymeric carrier.

5154. The device of item 4995 wherein the anti-scarring agent inhibits adhesion between the device and a host into which the device is implanted.

5155. The device of item 4995 wherein the device delivers the anti-scarring agent locally to tissue proximate to the device.

5156. The device of item 4995, further comprising a coating, wherein the coating comprises the anti-scarring agent.

5157. The device of item 4995, further comprising a coating, wherein the coating is disposed on a surface of the device.

5158. The device of item 4995, further comprising a coating, wherein the coating directly contacts the device.

5159. The device of item 4995, further comprising a coating, wherein the coating indirectly contacts the device.

5160. The device of item 4995, further comprising a coating, wherein the coating partially covers the device.

5161. The device of item 4995, further comprising a coating, wherein the coating completely covers the device.

5162. The device of item 4995, further comprising a coating, wherein the coating is a uniform coating.

5163. The device of item 4995, further comprising a coating, wherein the coating is a non-uniform coating.

5164. The device of item 4995, further comprising a coating, wherein the coating is a discontinuous coating.

5165. The device of item 4995, further comprising a coating, wherein the coating is a patterned coating.

5166. The device of item 4995, further comprising a coating, wherein the coating has a thickness of 100 μm or less.

5167. The device of item 4995, further comprising a coating, wherein the coating has a thickness of 10 μm or less.

5168. The device of item 4995, further comprising a coating, wherein the coating adheres to the surface of the device upon deployment of the device.

5169. The device of item 4995, further comprising a coating, wherein the coating is stable at room temperature for a period of 1 year.

5170. The device of item 4995, further comprising a coating, wherein the anti-scarring agent is present in the coating in an amount ranging between about 0.0001% to about 1% by weight.

5171. The device of item 4995, further comprising a coating, wherein the anti-scarring agent is present in the coating in an amount ranging between about 1% to about 10% by weight.

5172. The device of item 4995, further comprising a coating, wherein the anti-scarring agent is present in the coating in an amount ranging between about 10% to about 25% by weight.

5173. The device of item 4995, further comprising a coating, wherein the anti-scarring agent is present in the coating in an amount ranging between about 25% to about 70% by weight.

5174. The device of item 4995, further comprising a coating, wherein the coating further comprises a polymer.

5175. The device of item 4995, further comprising a first coating having a first composition and the second coating having a second composition.

5176. The device of item 4995, further comprising a first coating having a first composition and the second coating having a second composition, wherein the first composition and the second composition are different.

5177. The device of item 4995, further comprising a polymer.

5178. The device of item 4995, further comprising a polymeric carrier.

5179. The device of item 4995, further comprising a polymeric carrier, wherein the polymeric carrier comprises a copolymer.

5180. The device of item 4995, further comprising a polymeric carrier, wherein the polymeric carrier comprises a block copolymer.

5181. The device of item 4995, further comprising a polymeric carrier, wherein the polymeric carrier comprises a random copolymer.

5182. The device of item 4995, further comprising a polymeric carrier, wherein the polymeric carrier comprises a biodegradable polymer.

5183. The device of item 4995, further comprising a polymeric carrier, wherein the polymeric carrier comprises a non-biodegradable polymer.

5184. The device of item 4995, further comprising a polymeric carrier, wherein the polymeric carrier comprises a hydrophilic polymer.

5185. The device of item 4995, further comprising a polymeric carrier, wherein the polymeric carrier comprises a hydrophobic polymer.

5186. The device of item 4995, further comprising a polymeric carrier, wherein the polymeric carrier comprises a polymer having hydrophilic domains.

5187. The device of item 4995, further comprising a polymeric carrier, wherein the polymeric carrier comprises a polymer having hydrophobic domains.

5188. The device of item 4995, further comprising a polymeric carrier, wherein the polymeric carrier comprises a non-conductive polymer.

5189. The device of item 4995, further comprising a polymeric carrier, wherein the polymeric carrier comprises an elastomer.

5190. The device of item 4995, further comprising a polymeric carrier, wherein the polymeric carrier comprises a hydrogel.

5191. The device of item 4995, further comprising a polymeric carrier, wherein the polymeric carrier comprises a silicone polymer.

5192. The device of item 4995, further comprising a polymeric carrier, wherein the polymeric carrier comprises a hydrocarbon polymer.

5193. The device of item 4995, further comprising a polymeric carrier, wherein the polymeric carrier comprises a styrene-derived polymer.

5194. The device of item 4995, further comprising a polymeric carrier, wherein the polymeric carrier comprises a butadiene polymer.

5195. The device of item 4995, further comprising a polymeric carrier, wherein the polymeric carrier comprises a macromer.

5196. The device of item 4995, further comprising a polymeric carrier, wherein the polymeric carrier comprises a poly(ethylene glycol)polymer.

5197. The device of item 4995, further comprising a polymeric carrier, wherein the polymeric carrier comprises an amorphous polymer.

5198. The device of item 4995, further comprising a lubricious coating.

5199. The device of item 4995 wherein the anti-scarring agent is located within pores or holes of the device.

5200. The device of item 4995 wherein the anti-scarring agent is located within a channel, lumen, or divet of the device.

5201. The device of item 4995, further comprising a second pharmaceutically active agent.

5202. The device of item 4995, further comprising an anti-inflammatory agent.

5203. The device of item 4995, further comprising an agent that inhibits infection.

5204. The device of item 4995, further comprising an agent that inhibits infection, wherein the agent is an anthracycline.

5205. The device of item 4995, further comprising an agent that inhibits infection, wherein the agent is doxorubicin.

5206. The device of item 4995, further comprising an agent that inhibits infection, wherein the agent is mitoxantrone.

5207. The device of item 4995, further comprising an agent that inhibits infection, wherein the agent is a fluoropyrimidine.

5208. The device of item 4995, further comprising an agent that inhibits infection, wherein the agent is 5-fluorouracil (5-FU).

5209. The device of item 4995, further comprising an agent that inhibits infection, wherein the agent is a folic acid antagonist.

5210. The device of item 4995, further comprising an agent that inhibits infection, wherein the agent is methotrexate.

5211. The device of item 4995, further comprising an agent that inhibits infection, wherein the agent is a podophylotoxin.

5212. The device of item 4995, further comprising an agent that inhibits infection, wherein the agent is etoposide.

5213. The device of item 4995, further comprising an agent that inhibits infection, wherein the agent is a camptothecin.

5214. The device of item 4995, further comprising an agent that inhibits infection, wherein the agent is a hydroxyurea.

5215. The device of item 4995, further comprising an agent that inhibits infection, wherein the agent is a platinum complex.

5216. The device of item 4995, further comprising an agent that inhibits infection, wherein the agent is cisplatin.

5217. The device of item 4995, further comprising an anti-thrombotic agent.

5218. The device of item 4995, further comprising a visualization agent.

5219. The device of item 4995, further comprising a visualization agent, wherein the visualization agent is a radiopaque material, wherein the radiopaque material comprises a metal, a halogenated compound, or a barium containing compound.

5220. The device of item 4995, further comprising a visualization agent, wherein the visualization agent is a radiopaque material, wherein the radiopaque material comprises barium, tantalum, or technetium.

5221. The device of item 4995, further comprising a visualization agent, wherein the visualization agent is a MRI responsive material.

5222. The device of item 4995, further comprising a visualization agent, wherein the visualization agent comprises a gadolinium chelate.

5223. The device of item 4995, further comprising a visualization agent, wherein the visualization agent comprises iron, magnesium, manganese, copper, or chromium.

5224. The device of item 4995, further comprising a visualization agent, wherein the visualization agent comprises an iron oxide compound.

5225. The device of item 4995, further comprising a visualization agent, wherein the visualization agent comprises a dye, pigment, or colorant.

5226. The device of item 4995, further comprising an echogenic material.

5227. The device of item 4995, further comprising an echogenic material, wherein the echogenic material is in the form of a coating.

5228. The device of item 4995 wherein the device is sterile.

5229. The device of item 4995 wherein the anti-scarring agent is released into tissue in the vicinity of the device after deployment of the device.

5230. The device of item 4995 wherein the anti-scarring agent is released into tissue in the vicinity of the device after deployment of the device, wherein the tissue is connective tissue.

5231. The device of item 4995 wherein the anti-scarring agent is released into tissue in the vicinity of the device after deployment of the device, wherein the tissue is muscle tissue.

5232. The device of item 4995 wherein the anti-scarring agent is released into tissue in the vicinity of the device after deployment of the device, wherein the tissue is nerve tissue.

5233. The device of item 4995 wherein the anti-scarring agent is released into tissue in the vicinity of the device after deployment of the device, wherein the tissue is epithelium tissue.

5234. The device of item 4995 wherein the anti-scarring agent is released in effective concentrations from the device over a period ranging from the time of deployment of the device to about 1 year.

5235. The device of item 4995 wherein the anti-scarring agent is released in effective concentrations from the device over a period ranging from about 1 month to 6 months.

5236. The device of item 4995 wherein the anti-scarring agent is released in effective concentrations from the device over a period ranging from about 1-90 days.

5237. The device of item 4995 wherein the anti-scarring agent is released in effective concentrations from the device at a constant rate.

5238. The device of item 4995 wherein the anti-scarring agent is released in effective concentrations from the device at an increasing rate.

5239. The device of item 4995 wherein the anti-scarring agent is released in effective concentrations from the device at a decreasing rate.

5240. The device of item 4995 wherein the anti-scarring agent is released in effective concentrations from the composition comprising the anti-scarring agent by diffusion over a period ranging from the time of deployment of the device to about 90 days.

5241. The device of item 4995 wherein the anti-scarring agent is released in effective concentrations from the composition comprising the anti-scarring agent by erosion of the composition over a period ranging from the time of deployment of the device to about 90 days.

5242. The device of item 4995 wherein the device comprises about 0.01 μg to about 10 μg of the anti-scarring agent.

5243. The device of item 4995 wherein the device comprises about 10 μg to about 10 mg of the anti-scarring agent.

5244. The device of item 4995 wherein the device comprises about 10 mg to about 250 mg of the anti-scarring agent.

5245. The device of item 4995 wherein the device comprises about 250 mg to about 1000 mg of the anti-scarring agent.

5246. The device of item 4995 wherein the device comprises about 1000 mg to about 2500 mg of the anti-scarring agent.

5247. The device of item 4995 wherein a surface of the device comprises less than 0.01 μg of the anti-scarring agent per mm2 of device surface to which the anti-scarring agent is applied.

5248. The device of item 4995 wherein a surface of the device comprises about 0.01 μg to about 1 μg of the anti-scarring agent per mm2 of device surface to which the anti-scarring agent is applied.

5249. The device of item 4995 wherein a surface of the device comprises about 1 μg to about 10 μg of the anti-scarring agent per mm2 of device surface to which the anti-scarring agent is applied.

5250. The device of item 4995 wherein a surface of the device comprises about 10 μg to about 250 μg of the anti-scarring agent per mm2 of device surface to which the anti-scarring agent is applied.

5251. The device of item 4995 wherein a surface of the device comprises about 250 μg to about 1000 μg of the anti-scarring agent of anti-scarring agent per mm2 of device surface to which the anti-scarring agent is applied.

5252. The device of item 4995 wherein a surface of the device comprises about 1000 μg to about 2500 μg of the anti-scarring agent per mm2 of device surface to which the anti-scarring agent is applied.

5253. A method for inhibiting scarring comprising placing an intravascular implant and an anti-scarring agent or a composition comprising an anti-scarring agent into an animal host, wherein the agent inhibits scarring.

5254. The method of item 5253 wherein the agent inhibits cell regeneration.

5255. The method of item 5253 wherein the agent inhibits angiogenesis.

5256. The method of item 5253 wherein the agent inhibits fibroblast migration.

5257. The method of item 5253 wherein the agent inhibits fibroblast proliferation.

5258. The method of item 5253 wherein the agent inhibits deposition of extracellular matrix.

5259. The method of item 5253 wherein the agent inhibits tissue remodeling.

5260. The method of item 5253 wherein the agent is an angiogenesis inhibitor.

5261. The method of item 5253 wherein the agent is a 5-lipoxygenase inhibitor or antagonist.

5262. The method of item 5253 wherein the agent is a chemokine receptor antagonist.

5263. The method of item 5253 wherein the agent is a cell cycle inhibitor.

5264. The method of item 5253 wherein the agent is a taxane.

5265. The method of item 5253 wherein the agent is an anti-microtubule agent.

5266. The method of item 5253 wherein the agent is paclitaxel.

5267. The method of item 5253 wherein the agent is not paclitaxel.

5268. The method of item 5253 wherein the agent is an analogue or derivative of paclitaxel.

5269. The method of item 5253 wherein the agent is a vinca alkaloid.

5270. The method of item 5253 wherein the agent is camptothecin or an analogue or derivative thereof.

5271. The method of item 5253 wherein the agent is a podophyllotoxin.

5272. The method of item 5253 wherein the agent is a podophyllotoxin, wherein the podophyllotoxin is etoposide or an analogue or derivative thereof.

5273. The method of item 5253 wherein the agent is an anthracycline.

5274. The method of item 5253 wherein the agent is an anthracycline, wherein the anthracycline is doxorubicin or an analogue or derivative thereof.

5275. The method of item 5253 wherein the agent is an anthracycline, wherein the anthracycline is mitoxantrone or an analogue or derivative thereof.

5276. The method of item 5253 wherein the agent is a platinum compound.

5277. The method of item 5253 wherein the agent is a nitrosourea.

5278. The method of item 5253 wherein the agent is a nitroimidazole.

5279. The method of item 5253 wherein the agent is a folic acid antagonist.

5280. The method of item 5253 wherein the agent is a cytidine analogue.

5281. The method of item 5253 wherein the agent is a pyrimidine analogue.

5282. The method of item 5253 wherein the agent is a fluoropyrimidine analogue.

5283. The method of item 5253 wherein the agent is a purine analogue.

5284. The method of item 5253 wherein the agent is a nitrogen mustard or an analogue or derivative thereof.

5285. The method of item 5253 wherein the agent is a hydroxyurea.

5286. The method of item 5253 wherein the agent is a mytomicin or an analogue or derivative thereof.

5287. The method of item 5253 wherein the agent is an alkyl sulfonate.

5288. The method of item 5253 wherein the agent is a benzamide or an analogue or derivative thereof.

5289. The method of item 5253 wherein the agent is a nicotinamide or an analogue or derivative thereof.

5290. The method of item 5253 wherein the agent is a halogenated sugar or an analogue or derivative thereof.

5291. The method of item 5253 wherein the agent is a DNA alkylating agent.

5292. The method of item 5253 wherein the agent is an anti-microtubule agent.

5293. The method of item 5253 wherein the agent is a topoisomerase inhibitor.

5294. The method of item 5253 wherein the agent is a DNA cleaving agent.

5295. The method of item 5253 wherein the agent is an antimetabolite.

5296. The method of item 5253 wherein the agent inhibits adenosine deaminase.

5297. The method of item 5253 wherein the agent inhibits purine ring synthesis.

5298. The method of item 5253 wherein the agent is a nucleotide interconversion inhibitor.

5299. The method of item 5253 wherein the agent inhibits dihydrofolate reduction.

5300. The method of item 5253 wherein the agent blocks thymidine monophosphate.

5301. The method of item 5253 wherein the agent causes DNA damage.

5302. The method of item 5253 wherein the agent is a DNA intercalation agent.

5303. The method of item 5253 wherein the agent is a RNA synthesis inhibitor.

5304. The method of item 5253 wherein the agent is a pyrimidine synthesis inhibitor.

5305. The method of item 5253 wherein the agent inhibits ribonucleotide synthesis or function.

5306. The method of item 5253 wherein the agent inhibits thymidine monophosphate synthesis or function.

5307. The method of item 5253 wherein the agent inhibits DNA synthesis.

5308. The method of item 5253 wherein the agent causes DNA adduct formation.

5309. The method of item 5253 wherein the agent inhibits protein synthesis.

5310. The method of item 5253 wherein the agent inhibits microtubule function.

5311. The method of item 5253 wherein the agent is a cyclin dependent protein kinase inhibitor.

5312. The method of item 5253 wherein the agent is an epidermal growth factor kinase inhibitor.

5313. The method of item 5253 wherein the agent is an elastase inhibitor.

5314. The method of item 5253 wherein the agent is a factor Xa inhibitor.

5315. The method of item 5253 wherein the agent is a farnesyltransferase inhibitor.

5316. The method of item 5253 wherein the agent is a fibrinogen antagonist.

5317. The method of item 5253 wherein the agent is a guanylate cyclase stimulant.

5318. The method of item 5253 wherein the agent is a heat shock protein 90 antagonist.

5319. The method of item 5253 wherein the agent is a heat shock protein 90 antagonist, wherein the heat shock protein 90 antagonist is geldanamycin or an analogue or derivative thereof.

5320. The method of item 5253 wherein the agent is a guanylate cyclase stimulant.

5321. The method of item 5253 wherein the agent is a HMGCoA reductase inhibitor.

5322. The method of item 5253 wherein the agent is a HMGCoA reductase inhibitor, wherein the HMGCoA reductase inhibitor is simvastatin or an analogue or derivative thereof.

5323. The method of item 5253 wherein the agent is a hydroorotate dehydrogenase inhibitor.

5324. The method of item 5253 wherein the agent is an IKK2 inhibitor.

5325. The method of item 5253 wherein the agent is an IL-1 antagonist.

5326. The method of item 5253 wherein the agent is an ICE antagonist.

5327. The method of item 5253 wherein the agent is an IRAK antagonist.

5328. The method of item 5253 wherein the agent is an IL-4 agonist.

5329. The method of item 5253 wherein the agent is an immunomodulatory agent.

5330. The method of item 5253 wherein the agent is sirolimus or an analogue or derivative thereof.

5331. The method of item 5253 wherein the agent is not sirolimus.

5332. The method of item 5253 wherein the agent is everolimus or an analogue or derivative thereof.

5333. The method of item 5253 wherein the agent is tacrolimus or an analogue or derivative thereof.

5334. The method of item 5253 wherein the agent is not tacrolimus.

5335. The method of item 5253 wherein the agent is biolmus or an analogue or derivative thereof.

5336. The method of item 5253 wherein the agent is tresperimus or an analogue or derivative thereof.

5337. The method of item 5253 wherein the agent is auranofin or an analogue or derivative thereof.

5338. The method of item 5253 wherein the agent is 27-0-demethylrapamycin or an analogue or derivative thereof.

5339. The method of item 5253 wherein the agent is gusperimus or an analogue or derivative thereof.

5340. The method of item 5253 wherein the agent is pimecrolimus or an analogue or derivative thereof.

5341. The method of item 5253 wherein the agent is ABT-578 or an analogue or derivative thereof.

5342. The method of item 5253 wherein the agent is an inosine monophosphate dehydrogenase (IMPDH) inhibitor.

5343. The method of item 5253 wherein the agent is an IMPDH inhibitor, wherein the IMPDH inhibitor is mycophenolic acid or an analogue or derivative thereof.

5344. The method of item 5253 wherein the agent is an IMPDH inhibitor, wherein the IMPDH inhibitor is 1-alpha-25 dihydroxy vitamin D3 or an analogue or derivative thereof.

5345. The method of item 5253 wherein the agent is a leukotriene inhibitor.

5346. The method of item 5253 wherein the agent is a MCP-1 antagonist.

5347. The method of item 5253 wherein the agent is a MMP inhibitor.

5348. The method of item 5253 wherein the agent is an NF kappa B inhibitor.

5349. The method of item 5253 wherein the agent is an NF kappa B inhibitor, wherein the NF kappa B inhibitor is Bay 11-7082.

5350. The method of item 5253 wherein the agent is an NO agonist.

5351. The method of item 5253 wherein the agent is a p38 MAP kinase inhibitor.

5352. The method of item 5253 wherein the agent is a p38 MAP kinase inhibitor, wherein the p38 MAP kinase inhibitor is SB 202190.

5353. The method of item 5253 wherein the agent is a phosphodiesterase inhibitor.

5354. The method of item 5253 wherein the agent is a TGF beta inhibitor.

5355. The method of item 5253 wherein the agent is a thromboxane A2 antagonist.

5356. The method of item 5253 wherein the agent is a TNFa antagonist.

5357. The method of item 5253 wherein the agent is a TACE inhibitor.

5358. The method of item 5253 wherein the agent is a tyrosine kinase inhibitor.

5359. The method of item 5253 wherein the agent is a vitronectin inhibitor.

5360. The method of item 5253 wherein the agent is a fibroblast growth factor inhibitor.

5361. The method of item 5253 wherein the agent is a protein kinase inhibitor.

5362. The method of item 5253 wherein the agent is a PDGF receptor kinase inhibitor.

5363. The method of item 5253 wherein the agent is an endothelial growth factor receptor kinase inhibitor.

5364. The method of item 5253 wherein the agent is a retinoic acid receptor antagonist.

5365. The method of item 5253 wherein the agent is a platelet derived growth factor receptor kinase inhibitor.

5366. The method of item 5253 wherein the agent is a fibronogin antagonist.

5367. The method of item 5253 wherein the agent is an antimycotic agent.

5368. The method of item 5253 wherein the agent is an antimycotic agent, wherein the antimycotic agent is sulconizole.

5369. The method of item 5253 wherein the agent is a bisphosphonate.

5370. The method of item 5253 wherein the agent is a phospholipase A1 inhibitor.

5371. The method of item 5253 wherein the agent is a histamine H1/H2/H3 receptor antagonist.

5372. The method of item 5253 wherein the agent is a macrolide antibiotic.

5373. The method of item 5253 wherein the agent is a GPIIb/IIIa receptor antagonist.

5374. The method of item 5253 wherein the agent is an endothelin receptor antagonist.

5375. The method of item 5253 wherein the agent is a peroxisome proliferator-activated receptor agonist.

5376. The method of item 5253 wherein the agent is an estrogen receptor agent.

5377. The method of item 5253 wherein the agent is a somastostatin analogue.

5378. The method of item 5253 wherein the agent is a neurokinin 1 antagonist.

5379. The method of item 5253 wherein the agent is a neurokinin 3 antagonist.

5380. The method of item 5253 wherein the agent is a VLA-4 antagonist.

5381. The method of item 5253 wherein the agent is an osteoclast inhibitor.

5382. The method of item 5253 wherein the agent is a DNA topoisomerase ATP hydrolyzing inhibitor.

5383. The method of item 5253 wherein the agent is an angiotensin I converting enzyme inhibitor.

5384. The method of item 5253 wherein the agent is an angiotensin II antagonist.

5385. The method of item 5253 wherein the agent is an enkephalinase inhibitor.

5386. The method of item 5253 wherein the agent is a peroxisome proliferator-activated receptor gamma agonist insulin sensitizer.

5387. The method of item 5253 wherein the agent is a protein kinase C inhibitor.

5388. The method of item 5253 wherein the agent is a ROCK (rho-associated kinase) inhibitor.

5389. The method of item 5253 wherein the agent is a CXCR3 inhibitor.

5390. The method of item 5253 wherein the agent is an Itk inhibitor.

5391. The method of item 5253 wherein the agent is a cytosolic phospholipase A2-alpha inhibitor.

5392. The method of item 5253 wherein the agent is a PPAR agonist.

5393. The method of item 5253 wherein the agent is an immunosuppressant.

5394. The method of item 5253 wherein the agent is an Erb inhibitor.

5395. The method of item 5253 wherein the agent is an apoptosis agonist.

5396. The method of item 5253 wherein the agent is a lipocortin agonist.

5397. The method of item 5253 wherein the agent is a VCAM-1 antagonist.

5398. The method of item 5253 wherein the agent is a collagen antagonist.

5399. The method of item 5253 wherein the agent is an alpha 2 integrin antagonist.

5400. The method of item 5253 wherein the agent is a TNF alpha inhibitor.

5401. The method of item 5253 wherein the agent is a nitric oxide inhibitor.

5402. The method of item 5253 wherein the agent is a cathepsin inhibitor.

5403. The method of item 5253 wherein the agent is not an anti-inflammatory agent.

5404. The method of item 5253 wherein the agent is not a steroid.

5405. The method of item 5253 wherein the agent is not a glucocorticosteroid.

5406. The method of item 5253 wherein the agent is not dexamethasone.

5407. The method of item 5253 wherein the agent is not an anti-infective agent.

5408. The method of item 5253 wherein the agent is not an antibiotic.

5409. The method of item 5253 wherein the agent is not an anti-fungal agent.

5410. The method of item 5253, wherein the composition comprises a polymer.

5411. The method of item 5253, wherein the composition comprises a polymer, and the polymer is, or comprises, a copolymer.

5412. The method of item 5253, wherein the composition comprises a polymer, and the polymer is, or comprises, a block copolymer.

5413. The method of item 5253, wherein the composition comprises a polymer, and the polymer is, or comprises, a random copolymer.

5414. The method of item 5253, wherein the composition comprises a polymer, and the polymer is, or comprises, a biodegradable polymer.

5415. The method of item 5253, wherein the composition comprises a polymer, and the polymer is, or comprises, a non-biodegradable polymer.

5416. The method of item 5253, wherein the composition comprises a polymer, and the polymer is, or comprises, a hydrophilic polymer.

5417. The method of item 5253, wherein the composition comprises a polymer, and the polymer is, or comprises, a hydrophobic polymer.

5418. The method of item 5253, wherein the composition comprises a polymer, and the polymer is, or comprises, a polymer having hydrophilic domains.

5419. The method of item 5253, wherein the composition comprises a polymer, and the polymer is, or comprises, a polymer having hydrophobic domains.

5420. The method of item 5253, wherein the composition comprises a polymer, and the polymer is, or comprises, a non-conductive polymer.

5421. The method of item 5253, wherein the composition comprises a polymer, and the polymer is, or comprises, an elastomer.

5422. The method of item 5253, wherein the composition comprises a polymer, and the polymer is, or comprises, a hydrogel.

5423. The method of item 5253, wherein the composition comprises a polymer, and the polymer is, or comprises, a silicone polymer.

5424. The method of item 5253, wherein the composition comprises a polymer, and the polymer is, or comprises, a hydrocarbon polymer.

5425. The method of item 5253, wherein the composition comprises a polymer, and the polymer is, or comprises, a styrene-derived polymer.

5426. The method of item 5253, wherein the composition comprises a polymer, and the polymer is, or comprises, a butadiene-derived polymer.

5427. The method of item 5253, wherein the composition comprises a polymer, and the polymer is, or comprises, a macromer.

5428. The method of item 5253, wherein the composition comprises a polymer, and the polymer is, or comprises, a poly(ethylene glycol)polymer.

5429. The method of item 5253, wherein the composition comprises a polymer, and the polymer is, or comprises, an amorphous polymer.

5430. The method of item 5253, wherein the composition further comprises a second pharmaceutically active agent.

5431. The method of item 5253, wherein the composition further comprises an anti-inflammatory agent.

5432. The method of item 5253, wherein the composition further comprises an agent that inhibits infection.

5433. The method of item 5253, wherein the composition further comprises an anthracycline.

5434. The method of item 5253, wherein the composition further comprises doxorubicin.

5435. The method of item 5253 wherein the composition further comprises mitoxantrone.

5436. The method of item 5253 wherein the composition further comprises a fluoropyrimidine.

5437. The method of item 5253, wherein the composition further comprises 5-fluorouracil (5-FU).

5438. The method of item 5253, wherein the composition further comprises a folic acid antagonist.

5439. The method of item 5253, wherein the composition further comprises methotrexate.

5440. The method of item 5253, wherein the composition further comprises a podophylotoxin.

5441. The method of item 5253, wherein the composition further comprises etoposide.

5442. The method of item 5253, wherein the composition further comprises camptothecin.

5443. The method of item 5253, wherein the composition further comprises a hydroxyurea.

5444. The method of item 5253, wherein the composition further comprises a platinum complex.

5445. The method of item 5253, wherein the composition further comprises cisplatin.

5446. The method of item 5253 wherein the composition further comprises an anti-thrombotic agent.

5447. The method of item 5253, wherein the composition further comprises a visualization agent.

5448. The method of item 5253, wherein the composition further comprises a visualization agent, and the visualization agent is a radiopaque material, wherein the radiopaque material comprises a metal, a halogenated compound, or a barium containing compound.

5449. The method of item 5253, wherein the composition further comprises a visualization agent, and the visualization agent is, or comprises, barium, tantalum, or technetium.

5450. The method of item 5253, wherein the composition further comprises a visualization agent, and the visualization agent is, or comprises, an MRI responsive material.

5451. The method of item 5253, wherein the composition further comprises a visualization agent, and the visualization agent is, or comprises, a gadolinium chelate.

5452. The method of item 5253, wherein the composition further comprises a visualization agent, and the visualization agent is, or comprises, iron, magnesium, manganese, copper, or chromium.

5453. The method of item 5253, wherein the composition further comprises a visualization agent, and the visualization agent is, or comprises, iron oxide compound.

5454. The method of item 5253, wherein the composition further comprises a visualization agent, and the visualization agent is, or comprises, a dye, pigment, or colorant.

5455. The method of item 5253 wherein the agent is released in effective concentrations from the composition comprising the agent by diffusion over a period ranging from the time of administration to about 90 days.

5456. The method of item 5253 wherein the agent is released in effective concentrations from the composition comprising the agent by erosion of the composition over a period ranging from the time of administration to about 90 days.

5457. The method of item 5253 wherein the composition further comprises an inflammatory cytokine.

5458. The method of item 5253 wherein the composition further comprises an agent that stimulates cell proliferation.

5459. The method of item 5253 wherein the composition further comprises a polymeric carrier.

5460. The method of item 5253 wherein the composition is in the form of a gel, paste, or spray.

5461. The method of item 5253 wherein the implant is partially constructed with the agent or the composition.

5462. The method of item 5253 wherein the implant is fully constructed with the agent or the composition.

5463. The method of item 5253 wherein the implant is impregnated with the agent or the composition.

5464. The method of item 5253, wherein the agent or the composition forms a coating, and the coating directly contacts the implant.

5465. The method of item 5253, wherein the agent or the composition forms a coating, and the coating indirectly contacts the implant.

5466. The method of item 5253 wherein the agent or the composition forms a coating, and the coating partially covers the implant.

5467. The method of item 5253, wherein the agent or the composition forms a coating, and the coating completely covers the implant.

5468. The method of item 5253 wherein the agent or the composition is located within pores or holes of the implant.

5469. The method of item 5253 wherein the agent or the composition is located within a channel, lumen, or divet of the implant.

5470. The method of item 5253 wherein the implant further comprising an echogenic material.

5471. The method of item 5253 wherein the implant further comprises an echogenic material, wherein the echogenic material is in the form of a coating.

5472. The method of item 5253 wherein the implant is sterile.

5473. The method of item 5253 wherein the agent is delivered from the implant, wherein the agent is released into tissue in the vicinity of the implant after deployment of the implant.

5474. The method of item 5253 wherein the agent is delivered from the implant, wherein the agent is released into tissue in the vicinity of the implant after deployment of the implant, wherein the tissue is connective tissue.

5475. The method of item 5253 wherein the agent is delivered from the implant, wherein the agent is released into tissue in the vicinity of the implant after deployment of the implant, wherein the tissue is muscle tissue.

5476. The method of item 5253 wherein the agent is delivered from the implant, wherein the agent is released into tissue in the vicinity of the implant after deployment of the implant, wherein the tissue is nerve tissue.

5477. The method of item 5253 wherein the agent is delivered from the implant, wherein the agent is released into tissue in the vicinity of the implant after deployment of the implant, wherein the tissue is epithelium tissue.

5478. The method of item 5253 wherein the agent is delivered from the implant, wherein the agent is released in effective concentrations from the implant over a period ranging from the time of deployment of the implant to about 1 year.

5479. The method of item 5253 wherein the agent is delivered from the implant, wherein the agent is released in effective concentrations from the implant over a period ranging from about 1 month to 6 months.

5480. The method of item 5253 wherein the agent is delivered from the implant, wherein the agent is released in effective concentrations from the implant over a period ranging from about 1-90 days.

5481. The method of item 5253 wherein the agent is delivered from the implant, wherein the agent is released in effective concentrations from the implant at a constant rate.

5482. The method of item 5253 wherein the agent is delivered from the implant, wherein the agent is released in effective concentrations from the implant at an increasing rate.

5483. The method of item 5253 wherein the agent is delivered from the implant, wherein the agent is released in effective concentrations from the implant at a decreasing rate.

5484. The method of item 5253 wherein the agent is delivered from the implant, wherein the implant comprises about 0.01 μg to about 10 μg of the agent.

5485. The method of item 5253 wherein the agent is delivered from the implant, wherein the implant comprises about 10 μg to about 10 mg of the agent.

5486. The method of item 5253 wherein the agent is delivered from the implant, wherein the implant comprises about 10 mg to about 250 mg of the agent.

5487. The method of item 5253 wherein the agent is delivered from the implant, wherein the implant comprises about 250 mg to about 1000 mg of the agent.

5488. The method of item 5253 wherein the agent is delivered from the implant, wherein the implant comprises about 1000 mg to about 2500 mg of the agent.

5489. The method of item 5253 wherein the agent is delivered from the implant, wherein a surface of the implant comprises less than 0.01 μg of the agent per mm2 of implant surface to which the agent is applied.

5490. The method of item 5253 wherein the agent is delivered from the implant, wherein a surface of the implant comprises about 0.01 μg to about 1 μg of the agent per mm2 of implant surface to which the agent is applied.

5491. The method of item 5253 wherein the agent is delivered from the implant, wherein a surface of the implant comprises about 1 μg to about 10 μg of the agent per mm2 of implant surface to which the agent is applied.

5492. The method of item 5253 wherein the agent is delivered from the implant, wherein a surface of the implant comprises about 10 μg to about 250 μg of the agent per mm2 of implant surface to which the agent is applied.

5493. The method of item 5253 wherein the agent is delivered from the implant, wherein a surface of the implant comprises about 250 μg to about 1000 μg of the agent per mm2 of implant surface to which the agent is applied.

5494. The method of item 5253 wherein the agent is delivered from the implant, wherein a surface of the implant comprises about 1000 μg to about 2500 μg of the agent per mm2 of implant surface to which the agent is applied.

5495. The method of item 5253, wherein the implant further comprises a coating, and the coating is a uniform coating.

5496. The method of item 5253, wherein the implant further comprises a coating, and the coating is a non-uniform coating.

5497. The method of item 5253, wherein the implant further comprises a coating, and the coating is a discontinuous coating.

5498. The method of item 5253, wherein the implant further comprises a coating, and the coating is a patterned coating.

5499. The method of item 5253, wherein the implant further comprises a coating, and the coating has a thickness of 100 μm or less.

5500. The method of item 5253, wherein the implant further comprises a coating, and the coating has a thickness of 10 μm or less.

5501. The method of item 5253, wherein the implant further comprises a coating, and the coating adheres to the surface of the implant upon deployment of the implant.

5502. The method of item 5253, wherein the implant further comprises a coating, and the coating is stable at room temperature for a period of at least 1 year.

5503. The method of item 5253, wherein the implant further comprises a coating, and the agent is present in the coating in an amount ranging between about 0.0001% to about 1% by weight.

5504. The method of item 5253, wherein the implant further comprises a coating, and the agent is present in the coating in an amount ranging between about 1% to about 10% by weight.

5505. The method of item 5253, wherein the implant further comprises a coating, and the agent is present in the coating in an amount ranging between about 10% to about 25% by weight.

5506. The method of item 5253, wherein the implant further comprises a coating, and the agent is present in the coating in an amount ranging between about 25% to about 70% by weight.

5507. The method of item 5253, wherein the implant further comprises a coating, and the coating comprises a polymer.

5508. The method of item 5253, wherein the implant comprises a first coating having a first composition and a second coating having a second composition.

5509. The method of item 5253, wherein the implant comprises a first coating having a first composition and a second coating having a second composition, wherein the first composition and the second composition are different.

5510. A method for inhibiting scarring comprising placing a vascular graft or wrap implant and an anti-scarring agent or a composition comprising an anti-scarring agent into an animal host, wherein the agent inhibits scarring.

5511. The method of item 5510 wherein the agent inhibits cell regeneration.

5512. The method of item 5510 wherein the agent inhibits angiogenesis.

5513. The method of item 5510 wherein the agent inhibits fibroblast migration.

5514. The method of item 5510 wherein the agent inhibits fibroblast proliferation.

5515. The method of item 5510 wherein the agent inhibits deposition of extracellular matrix.

5516. The method of item 5510 wherein the agent inhibits tissue remodeling.

5517. The method of item 5510 wherein the agent is an angiogenesis inhibitor.

5518. The method of item 5510 wherein the agent is a 5-lipoxygenase inhibitor or antagonist.

5519. The method of item 5510 wherein the agent is a chemokine receptor antagonist.

5520. The method of item 5510 wherein the agent is a cell cycle inhibitor.

5521. The method of item 5510 wherein the agent is a taxane.

5522. The method of item 5510 wherein the agent is an anti-microtubule agent.

5523. The method of item 5510 wherein the agent is paclitaxel.

5524. The method of item 5510 wherein the agent is not paclitaxel.

5525. The method of item 5510 wherein the agent is an analogue or derivative of paclitaxel.

5526. The method of item 5510 wherein the agent is a vinca alkaloid.

5527. The method of item 5510 wherein the agent is camptothecin or an analogue or derivative thereof.

5528. The method of item 5510 wherein the agent is a podophyllotoxin.

5529. The method of item 5510 wherein the agent is a podophyllotoxin, wherein the podophyllotoxin is etoposide or an analogue or derivative thereof.

5530. The method of item 5510 wherein the agent is an anthracycline.

5531. The method of item 5510 wherein the agent is an anthracycline, wherein the anthracycline is doxorubicin or an analogue or derivative thereof.

5532. The method of item 5510 wherein the agent is an anthracycline, wherein the anthracycline is mitoxantrone or an analogue or derivative thereof.

5533. The method of item 5510 wherein the agent is a platinum compound.

5534. The method of item 5510 wherein the agent is a nitrosourea.

5535. The method of item 5510 wherein the agent is a nitroimidazole.

5536. The method of item 5510 wherein the agent is a folic acid antagonist.

5537. The method of item 5510 wherein the agent is a cytidine analogue.

5538. The method of item 5510 wherein the agent is a pyrimidine analogue.

5539. The method of item 5510 wherein the agent is a fluoropyrimidine analogue.

5540. The method of item 5510 wherein the agent is a purine analogue.

5541. The method of item 5510 wherein the agent is a nitrogen mustard or an analogue or derivative thereof.

5542. The method of item 5510 wherein the agent is a hydroxyurea.

5543. The method of item 5510 wherein the agent is a mytomicin or an analogue or derivative thereof.

5544. The method of item 5510 wherein the agent is an alkyl sulfonate.

5545. The method of item 5510 wherein the agent is a benzamide or an analogue or derivative thereof.

5546. The method of item 5510 wherein the agent is a nicotinamide or an analogue or derivative thereof.

5547. The method of item 5510 wherein the agent is a halogenated sugar or an analogue or derivative thereof.

5548. The method of item 5510 wherein the agent is a DNA alkylating agent.

5549. The method of item 5510 wherein the agent is an anti-microtubule agent.

5550. The method of item 5510 wherein the agent is a topoisomerase inhibitor.

5551. The method of item 5510 wherein the agent is a DNA cleaving agent.

5552. The method of item 5510 wherein the agent is an antimetabolite.

5553. The method of item 5510 wherein the agent inhibits adenosine deaminase.

5554. The method of item 5510 wherein the agent inhibits purine ring synthesis.

5555. The method of item 5510 wherein the agent is a nucleotide interconversion inhibitor.

5556. The method of item 5510 wherein the agent inhibits dihydrofolate reduction.

5557. The method of item 5510 wherein the agent blocks thymidine monophosphate.

5558. The method of item 5510 wherein the agent causes DNA damage.

5559. The method of item 5510 wherein the agent is a DNA intercalation agent.

5560. The method of item 5510 wherein the agent is a RNA synthesis inhibitor.

5561. The method of item 5510 wherein the agent is a pyrimidine synthesis inhibitor.

5562. The method of item 5510 wherein the agent inhibits ribonucleotide synthesis or function.

5563. The method of item 5510 wherein the agent inhibits thymidine monophosphate synthesis or function.

5564. The method of item 5510 wherein the agent inhibits DNA synthesis.

5565. The method of item 5510 wherein the agent causes DNA adduct formation.

5566. The method of item 5510 wherein the agent inhibits protein synthesis.

5567. The method of item 5510 wherein the agent inhibits microtubule function.

5568. The method of item 5510 wherein the agent is a cyclin dependent protein kinase inhibitor.

5569. The method of item 5510 wherein the agent is an epidermal growth factor kinase inhibitor.

5570. The method of item 5510 wherein the agent is an elastase inhibitor.

5571. The method of item 5510 wherein the agent is a factor Xa inhibitor.

5572. The method of item 5510 wherein the agent is a farnesyltransferase inhibitor.

5573. The method of item 5510 wherein the agent is a fibrinogen antagonist.

5574. The method of item 5510 wherein the agent is a guanylate cyclase stimulant.

5575. The method of item 5510 wherein the agent is a heat shock protein 90 antagonist.

5576. The method of item 5510 wherein the agent is a heat shock protein 90 antagonist, wherein the heat shock protein 90 antagonist is geldanamycin or an analogue or derivative thereof.

5577. The method of item 5510 wherein the agent is a guanylate cyclase stimulant.

5578. The method of item 5510 wherein the agent is a HMGCoA reductase inhibitor.

5579. The method of item 5510 wherein the agent is a HMGCoA reductase inhibitor, wherein the HMGCoA reductase inhibitor is simvastatin or an analogue or derivative thereof.

5580. The method of item 5510 wherein the agent is a hydroorotate dehydrogenase inhibitor.

5581. The method of item 5510 wherein the agent is an IKK2 inhibitor.

5582. The method of item 5510 wherein the agent is an IL-1 antagonist.

5583. The method of item 5510 wherein the agent is an ICE antagonist.

5584. The method of item 5510 wherein the agent is an IRAK antagonist.

5585. The method of item 5510 wherein the agent is an IL-4 agonist.

5586. The method of item 5510 wherein the agent is an immunomodulatory agent.

5587. The method of item 5510 wherein the agent is sirolimus or an analogue or derivative thereof.

5588. The method of item 5510 wherein the agent is not sirolimus.

5589. The method of item 5510 wherein the agent is everolimus or an analogue or derivative thereof.

5590. The method of item 5510 wherein the agent is tacrolimus or an analogue or derivative thereof.

5591. The method of item 5510 wherein the agent is not tacrolimus.

5592. The method of item 5510 wherein the agent is biolmus or an analogue or derivative thereof.

5593. The method of item 5510 wherein the agent is tresperimus or an analogue or derivative thereof.

5594. The method of item 5510 wherein the agent is auranofin or an analogue or derivative thereof.

5595. The method of item 5510 wherein the agent is 27-0-demethylrapamycin or an analogue or derivative thereof.

5596. The method of item 5510 wherein the agent is gusperimus or an analogue or derivative thereof.

5597. The method of item 5510 wherein the agent is pimecrolimus or an analogue or derivative thereof.

5598. The method of item 5510 wherein the agent is ABT-578 or an analogue or derivative thereof.

5599. The method of item 5510 wherein the agent is an inosine monophosphate dehydrogenase (IMPDH) inhibitor.

5600. The method of item 5510 wherein the agent is an IMPDH inhibitor, wherein the IMPDH inhibitor is mycophenolic acid or an analogue or derivative thereof.

5601. The method of item 5510 wherein the agent is an IMPDH inhibitor, wherein the IMPDH inhibitor is 1-alpha-25 dihydroxy vitamin D3 or an analogue or derivative thereof.

5602. The method of item 5510 wherein the agent is a leukotriene inhibitor.

5603. The method of item 5510 wherein the agent is a MCP-1 antagonist.

5604. The method of item 5510 wherein the agent is a MMP inhibitor.

5605. The method of item 5510 wherein the agent is an NF kappa B inhibitor.

5606. The method of item 5510 wherein the agent is an NF kappa B inhibitor, wherein the NF kappa B inhibitor is Bay 11-7082.

5607. The method of item 5510 wherein the agent is an NO agonist.

5608. The method of item 5510 wherein the agent is a p38 MAP kinase inhibitor.

5609. The method of item 5510 wherein the agent is a p38 MAP kinase inhibitor, wherein the p38 MAP kinase inhibitor is SB 202190.

5610. The method of item 5510 wherein the agent is a phosphodiesterase inhibitor.

5611. The method of item 5510 wherein the agent is a TGF beta inhibitor.

5612. The method of item 5510 wherein the agent is a thromboxane A2 antagonist.

5613. The method of item 5510 wherein the agent is a TNFa antagonist.

5614. The method of item 5510 wherein the agent is a TACE inhibitor.

5615. The method of item 5510 wherein the agent is a tyrosine kinase inhibitor.

5616. The method of item 5510 wherein the agent is a vitronectin inhibitor.

5617. The method of item 5510 wherein the agent is a fibroblast growth factor inhibitor.

5618. The method of item 5510 wherein the agent is a protein kinase inhibitor.

5619. The method of item 5510 wherein the agent is a PDGF receptor kinase inhibitor.

5620. The method of item 5510 wherein the agent is an endothelial growth factor receptor kinase inhibitor.

5621. The method of item 5510 wherein the agent is a retinoic acid receptor antagonist.

5622. The method of item 5510 wherein the agent is a platelet derived growth factor receptor kinase inhibitor.

5623. The method of item 5510 wherein the agent is a fibronogin antagonist.

5624. The method of item 5510 wherein the agent is an antimycotic agent.

5625. The method of item 5510 wherein the agent is an antimycotic agent, wherein the antimycotic agent is sulconizole.

5626. The method of item 5510 wherein the agent is a bisphosphonate.

5627. The method of item 5510 wherein the agent is a phospholipase A1 inhibitor.

5628. The method of item 5510 wherein the agent is a histamine H1/H2/H3 receptor antagonist.

5629. The method of item 5510 wherein the agent is a macrolide antibiotic.

5630. The method of item 5510 wherein the agent is a GPIIb/IIIa receptor antagonist.

5631. The method of item 5510 wherein the agent is an endothelin receptor antagonist.

5632. The method of item 5510 wherein the agent is a peroxisome proliferator-activated receptor agonist.

5633. The method of item 5510 wherein the agent is an estrogen receptor agent.

5634. The method of item 5510 wherein the agent is a somastostatin analogue.

5635. The method of item 5510 wherein the agent is a neurokinin 1 antagonist.

5636. The method of item 5510 wherein the agent is a neurokinin 3 antagonist.

5637. The method of item 5510 wherein the agent is a VLA-4 antagonist.

5638. The method of item 5510 wherein the agent is an osteoclast inhibitor.

5639. The method of item 5510 wherein the agent is a DNA topoisomerase ATP hydrolyzing inhibitor.

5640. The method of item 5510 wherein the agent is an angiotensin I converting enzyme inhibitor.

5641. The method of item 5510 wherein the agent is an angiotensin II antagonist.

5642. The method of item 5510 wherein the agent is an enkephalinase inhibitor.

5643. The method of item 5510 wherein the agent is a peroxisome proliferator-activated receptor gamma agonist insulin sensitizer.

5644. The method of item 5510 wherein the agent is a protein kinase C inhibitor.

5645. The method of item 5510 wherein the agent is a ROCK (rho-associated kinase) inhibitor.

5646. The method of item 5510 wherein the agent is a CXCR3 inhibitor.

5647. The method of item 5510 wherein the agent is an Itk inhibitor.

5648. The method of item 5510 wherein the agent is a cytosolic phospholipase A2-alpha inhibitor.

5649. The method of item 5510 wherein the agent is a PPAR agonist.

5650. The method of item 5510 wherein the agent is an immunosuppressant.

5651. The method of item 5510 wherein the agent is an Erb inhibitor.

5652. The method of item 5510 wherein the agent is an apoptosis agonist.

5653. The method of item 5510 wherein the agent is a lipocortin agonist.

5654. The method of item 5510 wherein the agent is a VCAM-1 antagonist.

5655. The method of item 5510 wherein the agent is a collagen antagonist.

5656. The method of item 5510 wherein the agent is an alpha 2 integrin antagonist.

5657. The method of item 5510 wherein the agent is a TNF alpha inhibitor.

5658. The method of item 5510 wherein the agent is a nitric oxide inhibitor.

5659. The method of item 5510 wherein the agent is a cathepsin inhibitor.

5660. The method of item 5510 wherein the agent is not an anti-inflammatory agent.

5661. The method of item 5510 wherein the agent is not a steroid.

5662. The method of item 5510 wherein the agent is not a glucocorticosteroid.

5663. The method of item 5510 wherein the agent is not dexamethasone.

5664. The method of item 5510 wherein the agent is not an anti-infective agent.

5665. The method of item 5510 wherein the agent is not an antibiotic.

5666. The method of item 5510 wherein the agent is not an anti-fungal agent.

5667. The method of item 5510, wherein the composition comprises a polymer.

5668. The method of item 5510, wherein the composition comprises a polymer, and the polymer is, or comprises, a copolymer.

5669. The method of item 5510, wherein the composition comprises a polymer, and the polymer is, or comprises, a block copolymer.

5670. The method of item 5510, wherein the composition comprises a polymer, and the polymer is, or comprises, a random copolymer.

5671. The method of item 5510, wherein the composition comprises a polymer, and the polymer is, or comprises, a biodegradable polymer.

5672. The method of item 5510, wherein the composition comprises a polymer, and the polymer is, or comprises, a non-biodegradable polymer.

5673. The method of item 5510, wherein the composition comprises a polymer, and the polymer is, or comprises, a hydrophilic polymer.

5674. The method of item 5510, wherein the composition comprises a polymer, and the polymer is, or comprises, a hydrophobic polymer.

5675. The method of item 5510, wherein the composition comprises a polymer, and the polymer is, or comprises, a polymer having hydrophilic domains.

5676. The method of item 5510, wherein the composition comprises a polymer, and the polymer is, or comprises, a polymer having hydrophobic domains.

5677. The method of item 5510, wherein the composition comprises a polymer, and the polymer is, or comprises, a non-conductive polymer.

5678. The method of item 5510, wherein the composition comprises a polymer, and the polymer is, or comprises, an elastomer.

5679. The method of item 5510, wherein the composition comprises a polymer, and the polymer is, or comprises, a hydrogel.

5680. The method of item 5510, wherein the composition comprises a polymer, and the polymer is, or comprises, a silicone polymer.

5681. The method of item 5510, wherein the composition comprises a polymer, and the polymer is, or comprises, a hydrocarbon polymer.

5682. The method of item 5510, wherein the composition comprises a polymer, and the polymer is, or comprises, a styrene-derived polymer.

5683. The method of item 5510, wherein the composition comprises a polymer, and the polymer is, or comprises, a butadiene-derived polymer.

5684. The method of item 5510, wherein the composition comprises a polymer, and the polymer is, or comprises, a macromer.

5685. The method of item 5510, wherein the composition comprises a polymer, and the polymer is, or comprises, a poly(ethylene glycol)polymer.

5686. The method of item 5510, wherein the composition comprises a polymer, and the polymer is, or comprises, an amorphous polymer.

5687. The method of item 5510, wherein the composition further comprises a second pharmaceutically active agent.

5688. The method of item 5510, wherein the composition further comprises an anti-inflammatory agent.

5689. The method of item 5510, wherein the composition further comprises an agent that inhibits infection.

5690. The method of item 5510, wherein the composition further comprises an anthracycline.

5691. The method of item 5510, wherein the composition further comprises doxorubicin.

5692. The method of item 5510 wherein the composition further comprises mitoxantrone.

5693. The method of item 5510 wherein the composition further comprises a fluoropyrimidine.

5694. The method of item 5510, wherein the composition further comprises 5-fluorouracil (5-FU).

5695. The method of item 5510, wherein the composition further comprises a folic acid antagonist.

5696. The method of item 5510, wherein the composition further comprises methotrexate.

5697. The method of item 5510, wherein the composition further comprises a podophylotoxin.

5698. The method of item 5510, wherein the composition further comprises etoposide.

5699. The method of item 5510, wherein the composition further comprises camptothecin.

5700. The method of item 5510, wherein the composition further comprises a hydroxyurea.

5701. The method of item 5510, wherein the composition further comprises a platinum complex.

5702. The method of item 5510, wherein the composition further comprises cisplatin.

5703. The method of item 5510 wherein the composition further comprises an anti-thrombotic agent.

5704. The method of item 5510, wherein the composition further comprises a visualization agent.

5705. The method of item 5510, wherein the composition further comprises a visualization agent, and the visualization agent is a radiopaque material, wherein the radiopaque material comprises a metal, a halogenated compound, or a barium containing compound.

5706. The method of item 5510, wherein the composition further comprises a visualization agent, and the visualization agent is, or comprises, barium, tantalum, or technetium.

5707. The method of item 5510, wherein the composition further comprises a visualization agent, and the visualization agent is, or comprises, an MRI responsive material.

5708. The method of item 5510, wherein the composition further comprises a visualization agent, and the visualization agent is, or comprises, a gadolinium chelate.

5709. The method of item 5510, wherein the composition further comprises a visualization agent, and the visualization agent is, or comprises, iron, magnesium, manganese, copper, or chromium.

5710. The method of item 5510, wherein the composition further comprises a visualization agent, and the visualization agent is, or comprises, iron oxide compound.

5711. The method of item 5510, wherein the composition further comprises a visualization agent, and the visualization agent is, or comprises, a dye, pigment, or colorant.

5712. The method of item 5510 wherein the agent is released in effective concentrations from the composition comprising the agent by diffusion over a period ranging from the time of administration to about 90 days.

5713. The method of item 5510 wherein the agent is released in effective concentrations from the composition comprising the agent by erosion of the composition over a period ranging from the time of administration to about 90 days.

5714. The method of item 5510 wherein the composition further comprises an inflammatory cytokine.

5715. The method of item 5510 wherein the composition further comprises an agent that stimulates cell proliferation.

5716. The method of item 5510 wherein the composition further comprises a polymeric carrier.

5717. The method of item 5510 wherein the composition is in the form of a gel, paste, or spray.

5718. The method of item 5510 wherein the implant is partially constructed with the agent or the composition.

5719. The method of item 5510 wherein the implant is fully constructed with the agent or the composition.

5720. The method of item 5510 wherein the implant is impregnated with the agent or the composition.

5721. The method of item 5510, wherein the agent or the composition forms a coating, and the coating directly contacts the implant.

5722. The method of item 5510, wherein the agent or the composition forms a coating, and the coating indirectly contacts the implant.

5723. The method of item 5510 wherein the agent or the composition forms a coating, and the coating partially covers the implant.

5724. The method of item 5510, wherein the agent or the composition forms a coating, and the coating completely covers the implant.

5725. The method of item 5510 wherein the agent or the composition is located within pores or holes of the implant.

5726. The method of item 5510 wherein the agent or the composition is located within a channel, lumen, or divet of the implant.

5727. The method of item 5510 wherein the implant further comprising an echogenic material.

5728. The method of item 5510 wherein the implant further comprises an echogenic material, wherein the echogenic material is in the form of a coating.

5729. The method of item 5510 wherein the implant is sterile.

5730. The method of item 5510 wherein the agent is delivered from the implant, wherein the agent is released into tissue in the vicinity of the implant after deployment of the implant.

5731. The method of item 5510 wherein the agent is delivered from the implant, wherein the agent is released into tissue in the vicinity of the implant after deployment of the implant, wherein the tissue is connective tissue.

5732. The method of item 5510 wherein the agent is delivered from the implant, wherein the agent is released into tissue in the vicinity of the implant after deployment of the implant, wherein the tissue is muscle tissue.

5733. The method of item 5510 wherein the agent is delivered from the implant, wherein the agent is released into tissue in the vicinity of the implant after deployment of the implant, wherein the tissue is nerve tissue.

5734. The method of item 5510 wherein the agent is delivered from the implant, wherein the agent is released into tissue in the vicinity of the implant after deployment of the implant, wherein the tissue is epithelium tissue.

5735. The method of item 5510 wherein the agent is delivered from the implant, wherein the agent is released in effective concentrations from the implant over a period ranging from the time of deployment of the implant to about 1 year.

5736. The method of item 5510 wherein the agent is delivered from the implant, wherein the agent is released in effective concentrations from the implant over a period ranging from about 1 month to 6 months.

5737. The method of item 5510 wherein the agent is delivered from the implant, wherein the agent is released in effective concentrations from the implant over a period ranging from about 1-90 days.

5738. The method of item 5510 wherein the agent is delivered from the implant, wherein the agent is released in effective concentrations from the implant at a constant rate.

5739. The method of item 5510 wherein the agent is delivered from the implant, wherein the agent is released in effective concentrations from the implant at an increasing rate.

5740. The method of item 5510 wherein the agent is delivered from the implant, wherein the agent is released in effective concentrations from the implant at a decreasing rate.

5741. The method of item 5510 wherein the agent is delivered from the implant, wherein the implant comprises about 0.01 μg to about 10 μg of the agent.

5742. The method of item 5510 wherein the agent is delivered from the implant, wherein the implant comprises about 10 μg to about 10 mg of the agent.

5743. The method of item 5510 wherein the agent is delivered from the implant, wherein the implant comprises about 10 mg to about 250 mg of the agent.

5744. The method of item 5510 wherein the agent is delivered from the implant, wherein the implant comprises about 250 mg to about 1000 mg of the agent.

5745. The method of item 5510 wherein the agent is delivered from the implant, wherein the implant comprises about 1000 mg to about 2500 mg of the agent.

5746. The method of item 5510 wherein the agent is delivered from the implant, wherein a surface of the implant comprises less than 0.01 μg of the agent per mm2 of implant surface to which the agent is applied.

5747. The method of item 5510 wherein the agent is delivered from the implant, wherein a surface of the implant comprises about 0.01 μg to about 1 μg of the agent per mm2 of implant surface to which the agent is applied.

5748. The method of item 5510 wherein the agent is delivered from the implant, wherein a surface of the implant comprises about 1 μg to about 10 μg of the agent per mm2 of implant surface to which the agent is applied.

5749. The method of item 5510 wherein the agent is delivered from the implant, wherein a surface of the implant comprises about 10 μg to about 250 μg of the agent per mm2 of implant surface to which the agent is applied.

5750. The method of item 5510 wherein the agent is delivered from the implant, wherein a surface of the implant comprises about 250 μg to about 1000 μg of the agent per mm2 of implant surface to which the agent is applied.

5751. The method of item 5510 wherein the agent is delivered from the implant, wherein a surface of the implant comprises about 1000 μg to about 2500 μg of the agent per mm2 of implant surface to which the agent is applied.

5752. The method of item 5510, wherein the implant further comprises a coating, and the coating is a uniform coating.

5753. The method of item 5510, wherein the implant further comprises a coating, and the coating is a non-uniform coating.

5754. The method of item 5510, wherein the implant further comprises a coating, and the coating is a discontinuous coating.

5755. The method of item 5510, wherein the implant further comprises a coating, and the coating is a patterned coating.

5756. The method of item 5510, wherein the implant further comprises a coating, and the coating has a thickness of 100 μm or less.

5757. The method of item 5510, wherein the implant further comprises a coating, and the coating has a thickness of 10 μm or less.

5758. The method of item 5510, wherein the implant further comprises a coating, and the coating adheres to the surface of the implant upon deployment of the implant.

5759. The method of item 5510, wherein the implant further comprises a coating, and the coating is stable at room temperature for a period of at least 1 year.

5760. The method of item 5510, wherein the implant further comprises a coating, and the agent is present in the coating in an amount ranging between about 0.0001% to about 1% by weight.

5761. The method of item 5510, wherein the implant further comprises a coating, and the agent is present in the coating in an amount ranging between about 1% to about 10% by weight.

5762. The method of item 5510, wherein the implant further comprises a coating, and the agent is present in the coating in an amount ranging between about 10% to about 25% by weight.

5763. The method of item 5510, wherein the implant further comprises a coating, and the agent is present in the coating in an amount ranging between about 25% to about 70% by weight.

5764. The method of item 5510, wherein the implant further comprises a coating, and the coating comprises a polymer.

5765. The method of item 5510, wherein the implant comprises a first coating having a first composition and a second coating having a second composition.

5766. The method of item 5510, wherein the implant comprises a first coating having a first composition and a second coating having a second composition, wherein the first composition and the second composition are different.

5767. A method for inhibiting scarring comprising placing an implant for hemodialysis access and an anti-scarring agent or a composition comprising an anti-scarring agent into an animal host, wherein the agent inhibits scarring.

5768. The method of item 5767 wherein the agent inhibits cell regeneration.

5769. The method of item 5767 wherein the agent inhibits angiogenesis.

5770. The method of item 5767 wherein the agent inhibits fibroblast migration.

5771. The method of item 5767 wherein the agent inhibits fibroblast proliferation.

5772. The method of item 5767 wherein the agent inhibits deposition of extracellular matrix.

5773. The method of item 5767 wherein the agent inhibits tissue remodeling.

5774. The method of item 5767 wherein the agent is an angiogenesis inhibitor.

5775. The method of item 5767 wherein the agent is a 5-lipoxygenase inhibitor or antagonist.

5776. The method of item 5767 wherein the agent is a chemokine receptor antagonist.

5777. The method of item 5767 wherein the agent is a cell cycle inhibitor.

5778. The method of item 5767 wherein the agent is a taxane.

5779. The method of item 5767 wherein the agent is an anti-microtubule agent.

5780. The method of item 5767 wherein the agent is paclitaxel.

5781. The method of item 5767 wherein the agent is not paclitaxel.

5782. The method of item 5767 wherein the agent is an analogue or derivative of paclitaxel.

5783. The method of item 5767 wherein the agent is a vinca alkaloid.

5784. The method of item 5767 wherein the agent is camptothecin or an analogue or derivative thereof.

5785. The method of item 5767 wherein the agent is a podophyllotoxin.

5786. The method of item 5767 wherein the agent is a podophyllotoxin, wherein the podophyllotoxin is etoposide or an analogue or derivative thereof.

5787. The method of item 5767 wherein the agent is an anthracycline.

5788. The method of item 5767 wherein the agent is an anthracycline, wherein the anthracycline is doxorubicin or an analogue or derivative thereof.

5789. The method of item 5767 wherein the agent is an anthracycline, wherein the anthracycline is mitoxantrone or an analogue or derivative thereof.

5790. The method of item 5767 wherein the agent is a platinum compound.

5791. The method of item 5767 wherein the agent is a nitrosourea.

5792. The method of item 5767 wherein the agent is a nitroimidazole.

5793. The method of item 5767 wherein the agent is a folic acid antagonist.

5794. The method of item 5767 wherein the agent is a cytidine analogue.

5795. The method of item 5767 wherein the agent is a pyrimidine analogue.

5796. The method of item 5767 wherein the agent is a fluoropyrimidine analogue.

5797. The method of item 5767 wherein the agent is a purine analogue.

5798. The method of item 5767 wherein the agent is a nitrogen mustard or an analogue or derivative thereof.

5799. The method of item 5767 wherein the agent is a hydroxyurea.

5800. The method of item 5767 wherein the agent is a mytomicin or an analogue or derivative thereof.

5801. The method of item 5767 wherein the agent is an alkyl sulfonate.

5802. The method of item 5767 wherein the agent is a benzamide or an analogue or derivative thereof.

5803. The method of item 5767 wherein the agent is a nicotinamide or an analogue or derivative thereof.

5804. The method of item 5767 wherein the agent is a halogenated sugar or an analogue or derivative thereof.

5805. The method of item 5767 wherein the agent is a DNA alkylating agent.

5806. The method of item 5767 wherein the agent is an anti-microtubule agent.

5807. The method of item 5767 wherein the agent is a topoisomerase inhibitor.

5808. The method of item 5767 wherein the agent is a DNA cleaving agent.

5809. The method of item 5767 wherein the agent is an antimetabolite.

5810. The method of item 5767 wherein the agent inhibits adenosine deaminase.

5811. The method of item 5767 wherein the agent inhibits purine ring synthesis.

5812. The method of item 5767 wherein the agent is a nucleotide interconversion inhibitor.

5813. The method of item 5767 wherein the agent inhibits dihydrofolate reduction.

5814. The method of item 5767 wherein the agent blocks thymidine monophosphate.

5815. The method of item 5767 wherein the agent causes DNA damage.

5816. The method of item 5767 wherein the agent is a DNA intercalation agent.

5817. The method of item 5767 wherein the agent is a RNA synthesis inhibitor.

5818. The method of item 5767 wherein the agent is a pyrimidine synthesis inhibitor.

5819. The method of item 5767 wherein the agent inhibits ribonucleotide synthesis or function.

5820. The method of item 5767 wherein the agent inhibits thymidine monophosphate synthesis or function.

5821. The method of item 5767 wherein the agent inhibits DNA synthesis.

5822. The method of item 5767 wherein the agent causes DNA adduct formation.

5823. The method of item 5767 wherein the agent inhibits protein synthesis.

5824. The method of item 5767 wherein the agent inhibits microtubule function.

5825. The method of item 5767 wherein the agent is a cyclin dependent protein kinase inhibitor.

5826. The method of item 5767 wherein the agent is an epidermal growth factor kinase inhibitor.

5827. The method of item 5767 wherein the agent is an elastase inhibitor.

5828. The method of item 5767 wherein the agent is a factor Xa inhibitor.

5829. The method of item 5767 wherein the agent is a farnesyltransferase inhibitor.

5830. The method of item 5767 wherein the agent is a fibrinogen antagonist.

5831. The method of item 5767 wherein the agent is a guanylate cyclase stimulant.

5832. The method of item 5767 wherein the agent is a heat shock protein 90 antagonist.

5833. The method of item 5767 wherein the agent is a heat shock protein 90 antagonist, wherein the heat shock protein 90 antagonist is geldanamycin or an analogue or derivative thereof.

5834. The method of item 5767 wherein the agent is a guanylate cyclase stimulant.

5835. The method of item 5767 wherein the agent is a HMGCoA reductase inhibitor.

5836. The method of item 5767 wherein the agent is a HMGCoA reductase inhibitor, wherein the HMGCoA reductase inhibitor is simvastatin or an analogue or derivative thereof.

5837. The method of item 5767 wherein the agent is a hydroorotate dehydrogenase inhibitor.

5838. The method of item 5767 wherein the agent is an IKK2 inhibitor.

5839. The method of item 5767 wherein the agent is an IL-1 antagonist.

5840. The method of item 5767 wherein the agent is an ICE antagonist.

5841. The method of item 5767 wherein the agent is an IRAK antagonist.

5842. The method of item 5767 wherein the agent is an IL-4 agonist.

5843. The method of item 5767 wherein the agent is an immunomodulatory agent.

5844. The method of item 5767 wherein the agent is sirolimus or an analogue or derivative thereof.

5845. The method of item 5767 wherein the agent is not sirolimus.

5846. The method of item 5767 wherein the agent is everolimus or an analogue or derivative thereof.

5847. The method of item 5767 wherein the agent is tacrolimus or an analogue or derivative thereof.

5848. The method of item 5767 wherein the agent is not tacrolimus.

5849. The method of item 5767 wherein the agent is biolmus or an analogue or derivative thereof.

5850. The method of item 5767 wherein the agent is tresperimus or an analogue or derivative thereof.

5851. The method of item 5767 wherein the agent is auranofin or an analogue or derivative thereof.

5852. The method of item 5767 wherein the agent is 27-0-demethylrapamycin or an analogue or derivative thereof.

5853. The method of item 5767 wherein the agent is gusperimus or an analogue or derivative thereof.

5854. The method of item 5767 wherein the agent is pimecrolimus or an analogue or derivative thereof.

5855. The method of item 5767 wherein the agent is ABT-578 or an analogue or derivative thereof.

5856. The method of item 5767 wherein the agent is an inosine monophosphate dehydrogenase (IMPDH) inhibitor.

5857. The method of item 5767 wherein the agent is an IMPDH inhibitor, wherein the IMPDH inhibitor is mycophenolic acid or an analogue or derivative thereof.

5858. The method of item 5767 wherein the agent is an IMPDH inhibitor, wherein the IMPDH inhibitor is 1-alpha-25 dihydroxy vitamin D₃ or an analogue or derivative thereof.

5859. The method of item 5767 wherein the agent is a leukotriene inhibitor.

5860. The method of item 5767 wherein the agent is a MCP-1 antagonist.

5861. The method of item 5767 wherein the agent is a MMP inhibitor.

5862. The method of item 5767 wherein the agent is an NF kappa B inhibitor.

5863. The method of item 5767 wherein the agent is an NF kappa B inhibitor, wherein the NF kappa B inhibitor is Bay 11-7082.

5864. The method of item 5767 wherein the agent is an NO agonist.

5865. The method of item 5767 wherein the agent is a p38 MAP kinase inhibitor.

5866. The method of item 5767 wherein the agent is a p38 MAP kinase inhibitor, wherein the p38 MAP kinase inhibitor is SB 202190.

5867. The method of item 5767 wherein the agent is a phosphodiesterase inhibitor.

5868. The method of item 5767 wherein the agent is a TGF beta inhibitor.

5869. The method of item 5767 wherein the agent is a thromboxane A2 antagonist.

5870. The method of item 5767 wherein the agent is a TNFa antagonist.

5871. The method of item 5767 wherein the agent is a TACE inhibitor.

5872. The method of item 5767 wherein the agent is a tyrosine kinase inhibitor.

5873. The method of item 5767 wherein the agent is a vitronectin inhibitor.

5874. The method of item 5767 wherein the agent is a fibroblast growth factor inhibitor.

5875. The method of item 5767 wherein the agent is a protein kinase inhibitor.

5876. The method of item 5767 wherein the agent is a PDGF receptor kinase inhibitor.

5877. The method of item 5767 wherein the agent is an endothelial growth factor receptor kinase inhibitor.

5878. The method of item 5767 wherein the agent is a retinoic acid receptor antagonist.

5879. The method of item 5767 wherein the agent is a platelet derived growth factor receptor kinase inhibitor.

5880. The method of item 5767 wherein the agent is a fibronogin antagonist.

5881. The method of item 5767 wherein the agent is an antimycotic agent.

5882. The method of item 5767 wherein the agent is an antimycotic agent, wherein the antimycotic agent is sulconizole.

5883. The method of item 5767 wherein the agent is a bisphosphonate.

5884. The method of item 5767 wherein the agent is a phospholipase A1 inhibitor.

5885. The method of item 5767 wherein the agent is a histamine H1/H2/H3 receptor antagonist.

5886. The method of item 5767 wherein the agent is a macrolide antibiotic.

5887. The method of item 5767 wherein the agent is a GPIIb/IIIa receptor antagonist.

5888. The method of item 5767 wherein the agent is an endothelin receptor antagonist.

5889. The method of item 5767 wherein the agent is a peroxisome proliferator-activated receptor agonist.

5890. The method of item 5767 wherein the agent is an estrogen receptor agent.

5891. The method of item 5767 wherein the agent is a somastostatin analogue.

5892. The method of item 5767 wherein the agent is a neurokinin 1 antagonist.

5893. The method of item 5767 wherein the agent is a neurokinin 3 antagonist.

5894. The method of item 5767 wherein the agent is a VLA-4 antagonist.

5895. The method of item 5767 wherein the agent is an osteoclast inhibitor.

5896. The method of item 5767 wherein the agent is a DNA topoisomerase ATP hydrolyzing inhibitor.

5897. The method of item 5767 wherein the agent is an angiotensin I converting enzyme inhibitor.

5898. The method of item 5767 wherein the agent is an angiotensin II antagonist.

5899. The method of item 5767 wherein the agent is an enkephalinase inhibitor.

5900. The method of item 5767 wherein the agent is a peroxisome proliferator-activated receptor gamma agonist insulin sensitizer.

5901. The method of item 5767 wherein the agent is a protein kinase C inhibitor.

5902. The method of item 5767 wherein the agent is a ROCK (rho-associated kinase) inhibitor.

5903. The method of item 5767 wherein the agent is a CXCR3 inhibitor.

5904. The method of item 5767 wherein the agent is an Itk inhibitor.

5905. The method of item 5767 wherein the agent is a cytosolic phospholipase A₂-alpha inhibitor.

5906. The method of item 5767 wherein the agent is a PPAR agonist.

5907. The method of item 5767 wherein the agent is an immunosuppressant.

5908. The method of item 5767 wherein the agent is an Erb inhibitor.

5909. The method of item 5767 wherein the agent is an apoptosis agonist.

5910. The method of item 5767 wherein the agent is a lipocortin agonist.

5911. The method of item 5767 wherein the agent is a VCAM-1 antagonist.

5912. The method of item 5767 wherein the agent is a collagen antagonist.

5913. The method of item 5767 wherein the agent is an alpha 2 integrin antagonist.

5914. The method of item 5767 wherein the agent is a TNF alpha inhibitor.

5915. The method of item 5767 wherein the agent is a nitric oxide inhibitor.

5916. The method of item 5767 wherein the agent is a cathepsin inhibitor.

5917. The method of item 5767 wherein the agent is not an anti-inflammatory agent.

5918. The method of item 5767 wherein the agent is not a steroid.

5919. The method of item 5767 wherein the agent is not a glucocorticosteroid.

5920. The method of item 5767 wherein the agent is not dexamethasone.

5921. The method of item 5767 wherein the agent is not an anti-infective agent.

5922. The method of item 5767 wherein the agent is not an antibiotic.

5923. The method of item 5767 wherein the agent is not an anti-fungal agent.

5924. The method of item 5767, wherein the composition comprises a polymer.

5925. The method of item 5767, wherein the composition comprises a polymer, and the polymer is, or comprises, a copolymer.

5926. The method of item 5767, wherein the composition comprises a polymer, and the polymer is, or comprises, a block copolymer.

5927. The method of item 5767, wherein the composition comprises a polymer, and the polymer is, or comprises, a random copolymer.

5928. The method of item 5767, wherein the composition comprises a polymer, and the polymer is, or comprises, a biodegradable polymer.

5929. The method of item 5767, wherein the composition comprises a polymer, and the polymer is, or comprises, a non-biodegradable polymer.

5930. The method of item 5767, wherein the composition comprises a polymer, and the polymer is, or comprises, a hydrophilic polymer.

5931. The method of item 5767, wherein the composition comprises a polymer, and the polymer is, or comprises, a hydrophobic polymer.

5932. The method of item 5767, wherein the composition comprises a polymer, and the polymer is, or comprises, a polymer having hydrophilic domains.

5933. The method of item 5767, wherein the composition comprises a polymer, and the polymer is, or comprises, a polymer having hydrophobic domains.

5934. The method of item 5767, wherein the composition comprises a polymer, and the polymer is, or comprises, a non-conductive polymer.

5935. The method of item 5767, wherein the composition comprises a polymer, and the polymer is, or comprises, an elastomer.

5936. The method of item 5767, wherein the composition comprises a polymer, and the polymer is, or comprises, a hydrogel.

5937. The method of item 5767, wherein the composition comprises a polymer, and the polymer is, or comprises, a silicone polymer.

5938. The method of item 5767, wherein the composition comprises a polymer, and the polymer is, or comprises, a hydrocarbon polymer.

5939. The method of item 5767, wherein the composition comprises a polymer, and the polymer is, or comprises, a styrene-derived polymer.

5940. The method of item 5767, wherein the composition comprises a polymer, and the polymer is, or comprises, a butadiene-derived polymer.

5941. The method of item 5767, wherein the composition comprises a polymer, and the polymer is, or comprises, a macromer.

5942. The method of item 5767, wherein the composition comprises a polymer, and the polymer is, or comprises, a poly(ethylene glycol)polymer.

5943. The method of item 5767, wherein the composition comprises a polymer, and the polymer is, or comprises, an amorphous polymer.

5944. The method of item 5767, wherein the composition further comprises a second pharmaceutically active agent.

5945. The method of item 5767, wherein the composition further comprises an anti-inflammatory agent.

5946. The method of item 5767, wherein the composition further comprises an agent that inhibits infection.

5947. The method of item 5767, wherein the composition further comprises an anthracycline.

5948. The method of item 5767, wherein the composition further comprises doxorubicin.

5949. The method of item 5767 wherein the composition further comprises mitoxantrone.

5950. The method of item 5767 wherein the composition further comprises a fluoropyrimidine.

5951. The method of item 5767, wherein the composition further comprises 5-fluorouracil (5-FU).

5952. The method of item 5767, wherein the composition further comprises a folic acid antagonist.

5953. The method of item 5767, wherein the composition further comprises methotrexate.

5954. The method of item 5767, wherein the composition further comprises a podophylotoxin.

5955. The method of item 5767, wherein the composition further comprises etoposide.

5956. The method of item 5767, wherein the composition further comprises camptothecin.

5957. The method of item 5767, wherein the composition further comprises a hydroxyurea.

5958. The method of item 5767, wherein the composition further comprises a platinum complex.

5959. The method of item 5767, wherein the composition further comprises cisplatin.

5960. The method of item 5767 wherein the composition further comprises an anti-thrombotic agent.

5961. The method of item 5767, wherein the composition further comprises a visualization agent.

5962. The method of item 5767, wherein the composition further comprises a visualization agent, and the visualization agent is a radiopaque material, wherein the radiopaque material comprises a metal, a halogenated compound, or a barium containing compound.

5963. The method of item 5767, wherein the composition further comprises a visualization agent, and the visualization agent is, or comprises, barium, tantalum, or technetium.

5964. The method of item 5767, wherein the composition further comprises a visualization agent, and the visualization agent is, or comprises, an MRI responsive material.

5965. The method of item 5767, wherein the composition further comprises a visualization agent, and the visualization agent is, or comprises, a gadolinium chelate.

5966. The method of item 5767, wherein the composition further comprises a visualization agent, and the visualization agent is, or comprises, iron, magnesium, manganese, copper, or chromium.

5967. The method of item 5767, wherein the composition further comprises a visualization agent, and the visualization agent is, or comprises, iron oxide compound.

5968. The method of item 5767, wherein the composition further comprises a visualization agent, and the visualization agent is, or comprises, a dye, pigment, or colorant.

5969. The method of item 5767 wherein the agent is released in effective concentrations from the composition comprising the agent by diffusion over a period ranging from the time of administration to about 90 days.

5970. The method of item 5767 wherein the agent is released in effective concentrations from the composition comprising the agent by erosion of the composition over a period ranging from the time of administration to about 90 days.

5971. The method of item 5767 wherein the composition further comprises an inflammatory cytokine.

5972. The method of item 5767 wherein the composition further comprises an agent that stimulates cell proliferation.

5973. The method of item 5767 wherein the composition further comprises a polymeric carrier.

5974. The method of item 5767 wherein the composition is in the form of a gel, paste, or spray.

5975. The method of item 5767 wherein the implant is partially constructed with the agent or the composition.

5976. The method of item 5767 wherein the implant is fully constructed with the agent or the composition.

5977. The method of item 5767 wherein the implant is impregnated with the agent or the composition.

5978. The method of item 5767, wherein the agent or the composition forms a coating, and the coating directly contacts the implant.

5979. The method of item 5767, wherein the agent or the composition forms a coating, and the coating indirectly contacts the implant.

5980. The method of item 5767 wherein the agent or the composition forms a coating, and the coating partially covers the implant.

5981. The method of item 5767, wherein the agent or the composition forms a coating, and the coating completely covers the implant.

5982. The method of item 5767 wherein the agent or the composition is located within pores or holes of the implant.

5983. The method of item 5767 wherein the agent or the composition is located within a channel, lumen, or divet of the implant.

5984. The method of item 5767 wherein the implant further comprising an echogenic material.

5985. The method of item 5767 wherein the implant further comprises an echogenic material, wherein the echogenic material is in the form of a coating.

5986. The method of item 5767 wherein the implant is sterile.

5987. The method of item 5767 wherein the agent is delivered from the implant, wherein the agent is released into tissue in the vicinity of the implant after deployment of the implant.

5988. The method of item 5767 wherein the agent is delivered from the implant, wherein the agent is released into tissue in the vicinity of the implant after deployment of the implant, wherein the tissue is connective tissue.

5989. The method of item 5767 wherein the agent is delivered from the implant, wherein the agent is released into tissue in the vicinity of the implant after deployment of the implant, wherein the tissue is muscle tissue.

5990. The method of item 5767 wherein the agent is delivered from the implant, wherein the agent is released into tissue in the vicinity of the implant after deployment of the implant, wherein the tissue is nerve tissue.

5991. The method of item 5767 wherein the agent is delivered from the implant, wherein the agent is released into tissue in the vicinity of the implant after deployment of the implant, wherein the tissue is epithelium tissue.

5992. The method of item 5767 wherein the agent is delivered from the implant, wherein the agent is released in effective concentrations from the implant over a period ranging from the time of deployment of the implant to about 1 year.

5993. The method of item 5767 wherein the agent is delivered from the implant, wherein the agent is released in effective concentrations from the implant over a period ranging from about 1 month to 6 months.

5994. The method of item 5767 wherein the agent is delivered from the implant, wherein the agent is released in effective concentrations from the implant over a period ranging from about 1-90 days.

5995. The method of item 5767 wherein the agent is delivered from the implant, wherein the agent is released in effective concentrations from the implant at a constant rate.

5996. The method of item 5767 wherein the agent is delivered from the implant, wherein the agent is released in effective concentrations from the implant at an increasing rate.

5997. The method of item 5767 wherein the agent is delivered from the implant, wherein the agent is released in effective concentrations from the implant at a decreasing rate.

5998. The method of item 5767 wherein the agent is delivered from the implant, wherein the implant comprises about 0.01 μg to about 10 μg of the agent.

5999. The method of item 5767 wherein the agent is delivered from the implant, wherein the implant comprises about 10 μg to about 10 mg of the agent.

6000. The method of item 5767 wherein the agent is delivered from the implant, wherein the implant comprises about 10 mg to about 250 mg of the agent.

6001. The method of item 5767 wherein the agent is delivered from the implant, wherein the implant comprises about 250 mg to about 1000 mg of the agent.

6002. The method of item 5767 wherein the agent is delivered from the implant, wherein the implant comprises about 1000 mg to about 2500 mg of the agent.

6003. The method of item 5767 wherein the agent is delivered from the implant, wherein a surface of the implant comprises less than 0.01 μg of the agent per mm² of implant surface to which the agent is applied.

6004. The method of item 5767 wherein the agent is delivered from the implant, wherein a surface of the implant comprises about 0.01 μg to about 1 μg of the agent per mm² of implant surface to which the agent is applied.

6005. The method of item 5767 wherein the agent is delivered from the implant, wherein a surface of the implant comprises about 1 μg to about 10 μg of the agent per mm² of implant surface to which the agent is applied.

6006. The method of item 5767 wherein the agent is delivered from the implant, wherein a surface of the implant comprises about 10 μg to about 250 μg of the agent per mm² of implant surface to which the agent is applied.

6007. The method of item 5767 wherein the agent is delivered from the implant, wherein a surface of the implant comprises about 250 μg to about 1000 μg of the agent per mm² of implant surface to which the agent is applied.

6008. The method of item 5767 wherein the agent is delivered from the implant, wherein a surface of the implant comprises about 1000 μg to about 2500 μg of the agent per mm² of implant surface to which the agent is applied.

6009. The method of item 5767, wherein the implant further comprises a coating, and the coating is a uniform coating.

6010. The method of item 5767, wherein the implant further comprises a coating, and the coating is a non-uniform coating.

6011. The method of item 5767, wherein the implant further comprises a coating, and the coating is a discontinuous coating.

6012. The method of item 5767, wherein the implant further comprises a coating, and the coating is a patterned coating.

6013. The method of item 5767, wherein the implant further comprises a coating, and the coating has a thickness of 100 μm or less.

6014. The method of item 5767, wherein the implant further comprises a coating, and the coating has a thickness of 10 μm or less.

6015. The method of item 5767, wherein the implant further comprises a coating, and the coating adheres to the surface of the implant upon deployment of the implant.

6016. The method of item 5767, wherein the implant further comprises a coating, and the coating is stable at room temperature for a period of at least 1 year.

6017. The method of item 5767, wherein the implant further comprises a coating, and the agent is present in the coating in an amount ranging between about 0.0001% to about 1% by weight.

6018. The method of item 5767, wherein the implant further comprises a coating, and the agent is present in the coating in an amount ranging between about 1% to about 10% by weight.

6019. The method of item 5767, wherein the implant further comprises a coating, and the agent is present in the coating in an amount ranging between about 10% to about 25% by weight.

6020. The method of item 5767, wherein the implant further comprises a coating, and the agent is present in the coating in an amount ranging between about 25% to about 70% by weight.

6021. The method of item 5767, wherein the implant further comprises a coating, and the coating comprises a polymer.

6022. The method of item 5767, wherein the implant comprises a first coating having a first composition and a second coating having a second composition.

6023. The method of item 5767, wherein the implant comprises a first coating having a first composition and a second coating having a second composition, wherein the first composition and the second composition are different.

6024. A method for inhibiting scarring comprising placing an implant that provides an anastomotic connection (i.e., an anastomotic connector device) and an anti-scarring agent or a composition comprising an anti-scarring agent into an animal host, wherein the agent inhibits scarring.

6025. The method of item 6024 wherein the agent inhibits cell regeneration.

6026. The method of item 6024 wherein the agent inhibits angiogenesis.

6027. The method of item 6024 wherein the agent inhibits fibroblast migration.

6028. The method of item 6024 wherein the agent inhibits fibroblast proliferation.

6029. The method of item 6024 wherein the agent inhibits deposition of extracellular matrix.

6030. The method of item 6024 wherein the agent inhibits tissue remodeling.

6031. The method of item 6024 wherein the agent is an angiogenesis inhibitor.

6032. The method of item 6024 wherein the agent is a 5-lipoxygenase inhibitor or antagonist.

6033. The method of item 6024 wherein the agent is a chemokine receptor antagonist.

6034. The method of item 6024 wherein the agent is a cell cycle inhibitor.

6035. The method of item 6024 wherein the agent is a taxane.

6036. The method of item 6024 wherein the agent is an anti-microtubule agent.

6037. The method of item 6024 wherein the agent is paclitaxel.

6038. The method of item 6024 wherein the agent is not paclitaxel.

6039. The method of item 6024 wherein the agent is an analogue or derivative of paclitaxel.

6040. The method of item 6024 wherein the agent is a vinca alkaloid.

6041. The method of item 6024 wherein the agent is camptothecin or an analogue or derivative thereof.

6042. The method of item 6024 wherein the agent is a podophyllotoxin.

6043. The method of item 6024 wherein the agent is a podophyllotoxin, wherein the podophyllotoxin is etoposide or an analogue or derivative thereof.

6044. The method of item 6024 wherein the agent is an anthracycline.

6045. The method of item 6024 wherein the agent is an anthracycline, wherein the anthracycline is doxorubicin or an analogue or derivative thereof.

6046. The method of item 6024 wherein the agent is an anthracycline, wherein the anthracycline is mitoxantrone or an analogue or derivative thereof.

6047. The method of item 6024 wherein the agent is a platinum compound.

6048. The method of item 6024 wherein the agent is a nitrosourea.

6049. The method of item 6024 wherein the agent is a nitroimidazole.

6050. The method of item 6024 wherein the agent is a folic acid antagonist.

6051. The method of item 6024 wherein the agent is a cytidine analogue.

6052. The method of item 6024 wherein the agent is a pyrimidine analogue.

6053. The method of item 6024 wherein the agent is a fluoropyrimidine analogue.

6054. The method of item 6024 wherein the agent is a purine analogue.

6055. The method of item 6024 wherein the agent is a nitrogen mustard or an analogue or derivative thereof.

6056. The method of item 6024 wherein the agent is a hydroxyurea.

6057. The method of item 6024 wherein the agent is a mytomicin or an analogue or derivative thereof.

6058. The method of item 6024 wherein the agent is an alkyl sulfonate.

6059. The method of item 6024 wherein the agent is a benzamide or an analogue or derivative thereof.

6060. The method of item 6024 wherein the agent is a nicotinamide or an analogue or derivative thereof.

6061. The method of item 6024 wherein the agent is a halogenated sugar or an analogue or derivative thereof.

6062. The method of item 6024 wherein the agent is a DNA alkylating agent.

6063. The method of item 6024 wherein the agent is an anti-microtubule agent.

6064. The method of item 6024 wherein the agent is a topoisomerase inhibitor.

6065. The method of item 6024 wherein the agent is a DNA cleaving agent.

6066. The method of item 6024 wherein the agent is an antimetabolite.

6067. The method of item 6024 wherein the agent inhibits adenosine deaminase.

6068. The method of item 6024 wherein the agent inhibits purine ring synthesis.

6069. The method of item 6024 wherein the agent is a nucleotide interconversion inhibitor.

6070. The method of item 6024 wherein the agent inhibits dihydrofolate reduction.

6071. The method of item 6024 wherein the agent blocks thymidine monophosphate.

6072. The method of item 6024 wherein the agent causes DNA damage.

6073. The method of item 6024 wherein the agent is a DNA intercalation agent.

6074. The method of item 6024 wherein the agent is a RNA synthesis inhibitor.

6075. The method of item 6024 wherein the agent is a pyrimidine synthesis inhibitor.

6076. The method of item 6024 wherein the agent inhibits ribonucleotide synthesis or function.

6077. The method of item 6024 wherein the agent inhibits thymidine monophosphate synthesis or function.

6078. The method of item 6024 wherein the agent inhibits DNA synthesis.

6079. The method of item 6024 wherein the agent causes DNA adduct formation.

6080. The method of item 6024 wherein the agent inhibits protein synthesis.

6081. The method of item 6024 wherein the agent inhibits microtubule function.

6082. The method of item 6024 wherein the agent is a cyclin dependent protein kinase inhibitor.

6083. The method of item 6024 wherein the agent is an epidermal growth factor kinase inhibitor.

6084. The method of item 6024 wherein the agent is an elastase inhibitor.

6085. The method of item 6024 wherein the agent is a factor Xa inhibitor.

6086. The method of item 6024 wherein the agent is a farnesyltransferase inhibitor.

6087. The method of item 6024 wherein the agent is a fibrinogen antagonist.

6088. The method of item 6024 wherein the agent is a guanylate cyclase stimulant.

6089. The method of item 6024 wherein the agent is a heat shock protein 90 antagonist.

6090. The method of item 6024 wherein the agent is a heat shock protein 90 antagonist, wherein the heat shock protein 90 antagonist is geldanamycin or an analogue or derivative thereof.

6091. The method of item 6024 wherein the agent is a guanylate cyclase stimulant.

6092. The method of item 6024 wherein the agent is a HMGCoA reductase inhibitor.

6093. The method of item 6024 wherein the agent is a HMGCoA reductase inhibitor, wherein the HMGCoA reductase inhibitor is simvastatin or an analogue or derivative thereof.

6094. The method of item 6024 wherein the agent is a hydroorotate dehydrogenase inhibitor.

6095. The method of item 6024 wherein the agent is an IKK2 inhibitor.

6096. The method of item 6024 wherein the agent is an IL-1 antagonist.

6097. The method of item 6024 wherein the agent is an ICE antagonist.

6098. The method of item 6024 wherein the agent is an IRAK antagonist.

6099. The method of item 6024 wherein the agent is an IL-4 agonist.

6100. The method of item 6024 wherein the agent is an immunomodulatory agent.

6101. The method of item 6024 wherein the agent is sirolimus or an analogue or derivative thereof.

6102. The method of item 6024 wherein the agent is not sirolimus.

6103. The method of item 6024 wherein the agent is everolimus or an analogue or derivative thereof.

6104. The method of item 6024 wherein the agent is tacrolimus or an analogue or derivative thereof.

6105. The method of item 6024 wherein the agent is not tacrolimus.

6106. The method of item 6024 wherein the agent is biolmus or an analogue or derivative thereof.

6107. The method of item 6024 wherein the agent is tresperimus or an analogue or derivative thereof.

6108. The method of item 6024 wherein the agent is auranofin or an analogue or derivative thereof.

6109. The method of item 6024 wherein the agent is 27-0-demethylrapamycin or an analogue or derivative thereof.

6110. The method of item 6024 wherein the agent is gusperimus or an analogue or derivative thereof.

6111. The method of item 6024 wherein the agent is pimecrolimus or an analogue or derivative thereof.

6112. The method of item 6024 wherein the agent is ABT-578 or an analogue or derivative thereof.

6113. The method of item 6024 wherein the agent is an inosine monophosphate dehydrogenase (IMPDH) inhibitor.

6114. The method of item 6024 wherein the agent is an IMPDH inhibitor, wherein the IMPDH inhibitor is mycophenolic acid or an analogue or derivative thereof.

6115. The method of item 6024 wherein the agent is an IMPDH inhibitor, wherein the IMPDH inhibitor is 1-alpha-25 dihydroxy vitamin D₃ or an analogue or derivative thereof.

6116. The method of item 6024 wherein the agent is a leukotriene inhibitor.

6117. The method of item 6024 wherein the agent is a MCP-1 antagonist.

6118. The method of item 6024 wherein the agent is a MMP inhibitor.

6119. The method of item 6024 wherein the agent is an NF kappa B inhibitor.

6120. The method of item 6024 wherein the agent is an NF kappa B inhibitor, wherein the NF kappa B inhibitor is Bay 11-7082.

6121. The method of item 6024 wherein the agent is an NO agonist.

6122. The method of item 6024 wherein the agent is a p38 MAP kinase inhibitor.

6123. The method of item 6024 wherein the agent is a p38 MAP kinase inhibitor, wherein the p38 MAP kinase inhibitor is SB 202190.

6124. The method of item 6024 wherein the agent is a phosphodiesterase inhibitor.

6125. The method of item 6024 wherein the agent is a TGF beta inhibitor.

6126. The method of item 6024 wherein the agent is a thromboxane A2 antagonist.

6127. The method of item 6024 wherein the agent is a TNFa antagonist.

6128. The method of item 6024 wherein the agent is a TACE inhibitor.

6129. The method of item 6024 wherein the agent is a tyrosine kinase inhibitor.

6130. The method of item 6024 wherein the agent is a vitronectin inhibitor.

6131. The method of item 6024 wherein the agent is a fibroblast growth factor inhibitor.

6132. The method of item 6024 wherein the agent is a protein kinase inhibitor.

6133. The method of item 6024 wherein the agent is a PDGF receptor kinase inhibitor.

6134. The method of item 6024 wherein the agent is an endothelial growth factor receptor kinase inhibitor.

6135. The method of item 6024 wherein the agent is a retinoic acid receptor antagonist.

6136. The method of item 6024 wherein the agent is a platelet derived growth factor receptor kinase inhibitor.

6137. The method of item 6024 wherein the agent is a fibronogin antagonist.

6138. The method of item 6024 wherein the agent is an antimycotic agent.

6139. The method of item 6024 wherein the agent is an antimycotic agent, wherein the antimycotic agent is sulconizole.

6140. The method of item 6024 wherein the agent is a bisphosphonate.

6141. The method of item 6024 wherein the agent is a phospholipase A1 inhibitor.

6142. The method of item 6024 wherein the agent is a histamine H1/H2/H3 receptor antagonist.

6143. The method of item 6024 wherein the agent is a macrolide antibiotic.

6144. The method of item 6024 wherein the agent is a GPIIb/IIIa receptor antagonist.

6145. The method of item 6024 wherein the agent is an endothelin receptor antagonist.

6146. The method of item 6024 wherein the agent is a peroxisome proliferator-activated receptor agonist.

6147. The method of item 6024 wherein the agent is an estrogen receptor agent.

6148. The method of item 6024 wherein the agent is a somastostatin analogue.

6149. The method of item 6024 wherein the agent is a neurokinin 1 antagonist.

6150. The method of item 6024 wherein the agent is a neurokinin 3 antagonist.

6151. The method of item 6024 wherein the agent is a VLA-4 antagonist.

6152. The method of item 6024 wherein the agent is an osteoclast inhibitor.

6153. The method of item 6024 wherein the agent is a DNA topoisomerase ATP hydrolyzing inhibitor.

6154. The method of item 6024 wherein the agent is an angiotensin I converting enzyme inhibitor.

6155. The method of item 6024 wherein the agent is an angiotensin II antagonist.

6156. The method of item 6024 wherein the agent is an enkephalinase inhibitor.

6157. The method of item 6024 wherein the agent is a peroxisome proliferator-activated receptor gamma agonist insulin sensitizer.

6158. The method of item 6024 wherein the agent is a protein kinase C inhibitor.

6159. The method of item 6024 wherein the agent is a ROCK (rho-associated kinase) inhibitor.

6160. The method of item 6024 wherein the agent is a CXCR3 inhibitor.

6161. The method of item 6024 wherein the agent is an Itk inhibitor.

6162. The method of item 6024 wherein the agent is a cytosolic phospholipase A₂-alpha inhibitor.

6163. The method of item 6024 wherein the agent is a PPAR agonist.

6164. The method of item 6024 wherein the agent is an immunosuppressant.

6165. The method of item 6024 wherein the agent is an Erb inhibitor.

6166. The method of item 6024 wherein the agent is an apoptosis agonist.

6167. The method of item 6024 wherein the agent is a lipocortin agonist.

6168. The method of item 6024 wherein the agent is a VCAM-1 antagonist.

6169. The method of item 6024 wherein the agent is a collagen antagonist.

6170. The method of item 6024 wherein the agent is an alpha 2 integrin antagonist.

6171. The method of item 6024 wherein the agent is a TNF alpha inhibitor.

6172. The method of item 6024 wherein the agent is a nitric oxide inhibitor.

6173. The method of item 6024 wherein the agent is a cathepsin inhibitor.

6174. The method of item 6024 wherein the agent is not an anti-inflammatory agent.

6175. The method of item 6024 wherein the agent is not a steroid.

6176. The method of item 6024 wherein the agent is not a glucocorticosteroid.

6177. The method of item 6024 wherein the agent is not dexamethasone.

6178. The method of item 6024 wherein the agent is not an anti-infective agent.

6179. The method of item 6024 wherein the agent is not an antibiotic.

6180. The method of item 6024 wherein the agent is not an anti-fungal agent.

6181. The method of item 6024, wherein the composition comprises a polymer.

6182. The method of item 6024, wherein the composition comprises a polymer, and the polymer is, or comprises, a copolymer.

6183. The method of item 6024, wherein the composition comprises a polymer, and the polymer is, or comprises, a block copolymer.

6184. The method of item 6024, wherein the composition comprises a polymer, and the polymer is, or comprises, a random copolymer.

6185. The method of item 6024, wherein the composition comprises a polymer, and the polymer is, or comprises, a biodegradable polymer.

6186. The method of item 6024, wherein the composition comprises a polymer, and the polymer is, or comprises, a non-biodegradable polymer.

6187. The method of item 6024, wherein the composition comprises a polymer, and the polymer is, or comprises, a hydrophilic polymer.

6188. The method of item 6024, wherein the composition comprises a polymer, and the polymer is, or comprises, a hydrophobic polymer.

6189. The method of item 6024, wherein the composition comprises a polymer, and the polymer is, or comprises, a polymer having hydrophilic domains.

6190. The method of item 6024, wherein the composition comprises a polymer, and the polymer is, or comprises, a polymer having hydrophobic domains.

6191. The method of item 6024, wherein the composition comprises a polymer, and the polymer is, or comprises, a non-conductive polymer.

6192. The method of item 6024, wherein the composition comprises a polymer, and the polymer is, or comprises, an elastomer.

6193. The method of item 6024, wherein the composition comprises a polymer, and the polymer is, or comprises, a hydrogel.

6194. The method of item 6024, wherein the composition comprises a polymer, and the polymer is, or comprises, a silicone polymer.

6195. The method of item 6024, wherein the composition comprises a polymer, and the polymer is, or comprises, a hydrocarbon polymer.

6196. The method of item 6024, wherein the composition comprises a polymer, and the polymer is, or comprises, a styrene-derived polymer.

6197. The method of item 6024, wherein the composition comprises a polymer, and the polymer is, or comprises, a butadiene-derived polymer.

6198. The method of item 6024, wherein the composition comprises a polymer, and the polymer is, or comprises, a macromer.

6199. The method of item 6024, wherein the composition comprises a polymer, and the polymer is, or comprises, a poly(ethylene glycol)polymer.

6200. The method of item 6024, wherein the composition comprises a polymer, and the polymer is, or comprises, an amorphous polymer.

6201. The method of item 6024, wherein the composition further comprises a second pharmaceutically active agent.

6202. The method of item 6024, wherein the composition further comprises an anti-inflammatory agent.

6203. The method of item 6024, wherein the composition further comprises an agent that inhibits infection.

6204. The method of item 6024, wherein the composition further comprises an anthracycline.

6205. The method of item 6024, wherein the composition further comprises doxorubicin.

6206. The method of item 6024 wherein the composition further comprises mitoxantrone.

6207. The method of item 6024 wherein the composition further comprises a fluoropyrimidine.

6208. The method of item 6024, wherein the composition further comprises 5-fluorouracil (5-FU).

6209. The method of item 6024, wherein the composition further comprises a folic acid antagonist.

6210. The method of item 6024, wherein the composition further comprises methotrexate.

6211. The method of item 6024, wherein the composition further comprises a podophylotoxin.

6212. The method of item 6024, wherein the composition further comprises etoposide.

6213. The method of item 6024, wherein the composition further comprises camptothecin.

6214. The method of item 6024, wherein the composition further comprises a hydroxyurea.

6215. The method of item 6024, wherein the composition further comprises a platinum complex.

6216. The method of item 6024, wherein the composition further comprises cisplatin.

6217. The method of item 6024 wherein the composition further comprises an anti-thrombotic agent.

6218. The method of item 6024, wherein the composition further comprises a visualization agent.

6219. The method of item 6024, wherein the composition further comprises a visualization agent, and the visualization agent is a radiopaque material, wherein the radiopaque material comprises a metal, a halogenated compound, or a barium containing compound.

6220. The method of item 6024, wherein the composition further comprises a visualization agent, and the visualization agent is, or comprises, barium, tantalum, or technetium.

6221. The method of item 6024, wherein the composition further comprises a visualization agent, and the visualization agent is, or comprises, an MRI responsive material.

6222. The method of item 6024, wherein the composition further comprises a visualization agent, and the visualization agent is, or comprises, a gadolinium chelate.

6223. The method of item 6024, wherein the composition further comprises a visualization agent, and the visualization agent is, or comprises, iron, magnesium, manganese, copper, or chromium.

6224. The method of item 6024, wherein the composition further comprises a visualization agent, and the visualization agent is, or comprises, iron oxide compound.

6225. The method of item 6024, wherein the composition further comprises a visualization agent, and the visualization agent is, or comprises, a dye, pigment, or colorant.

6226. The method of item 6024 wherein the agent is released in effective concentrations from the composition comprising the agent by diffusion over a period ranging from the time of administration to about 90 days.

6227. The method of item 6024 wherein the agent is released in effective concentrations from the composition comprising the agent by erosion of the composition over a period ranging from the time of administration to about 90 days.

6228. The method of item 6024 wherein the composition further comprises an inflammatory cytokine.

6229. The method of item 6024 wherein the composition further comprises an agent that stimulates cell proliferation.

6230. The method of item 6024 wherein the composition further comprises a polymeric carrier.

6231. The method of item 6024 wherein the composition is in the form of a gel, paste, or spray.

6232. The method of item 6024 wherein the implant is partially constructed with the agent or the composition.

6233. The method of item 6024 wherein the implant is fully constructed with the agent or the composition.

6234. The method of item 6024 wherein the implant is impregnated with the agent or the composition.

6235. The method of item 6024, wherein the agent or the composition forms a coating, and the coating directly contacts the implant.

6236. The method of item 6024, wherein the agent or the composition forms a coating, and the coating indirectly contacts the implant.

6237. The method of item 6024 wherein the agent or the composition forms a coating, and the coating partially covers the implant.

6238. The method of item 6024, wherein the agent or the composition forms a coating, and the coating completely covers the implant.

6239. The method of item 6024 wherein the agent or the composition is located within pores or holes of the implant.

6240. The method of item 6024 wherein the agent or the composition is located within a channel, lumen, or divet of the implant.

6241. The method of item 6024 wherein the implant further comprising an echogenic material.

6242. The method of item 6024 wherein the implant further comprises an echogenic material, wherein the echogenic material is in the form of a coating.

6243. The method of item 6024 wherein the implant is sterile.

6244. The method of item 6024 wherein the agent is delivered from the implant, wherein the agent is released into tissue in the vicinity of the implant after deployment of the implant.

6245. The method of item 6024 wherein the agent is delivered from the implant, wherein the agent is released into tissue in the vicinity of the implant after deployment of the implant, wherein the tissue is connective tissue.

6246. The method of item 6024 wherein the agent is delivered from the implant, wherein the agent is released into tissue in the vicinity of the implant after deployment of the implant, wherein the tissue is muscle tissue.

6247. The method of item 6024 wherein the agent is delivered from the implant, wherein the agent is released into tissue in the vicinity of the implant after deployment of the implant, wherein the tissue is nerve tissue.

6248. The method of item 6024 wherein the agent is delivered from the implant, wherein the agent is released into tissue in the vicinity of the implant after deployment of the implant, wherein the tissue is epithelium tissue.

6249. The method of item 6024 wherein the agent is delivered from the implant, wherein the agent is released in effective concentrations from the implant over a period ranging from the time of deployment of the implant to about 1 year.

6250. The method of item 6024 wherein the agent is delivered from the implant, wherein the agent is released in effective concentrations from the implant over a period ranging from about 1 month to 6 months.

6251. The method of item 6024 wherein the agent is delivered from the implant, wherein the agent is released in effective concentrations from the implant over a period ranging from about 1-90 days.

6252. The method of item 6024 wherein the agent is delivered from the implant, wherein the agent is released in effective concentrations from the implant at a constant rate.

6253. The method of item 6024 wherein the agent is delivered from the implant, wherein the agent is released in effective concentrations from the implant at an increasing rate.

6254. The method of item 6024 wherein the agent is delivered from the implant, wherein the agent is released in effective concentrations from the implant at a decreasing rate.

6255. The method of item 6024 wherein the agent is delivered from the implant, wherein the implant comprises about 0.01 μg to about 10 μg of the agent.

6256. The method of item 6024 wherein the agent is delivered from the implant, wherein the implant comprises about 10 μg to about 10 mg of the agent.

6257. The method of item 6024 wherein the agent is delivered from the implant, wherein the implant comprises about 10 mg to about 250 mg of the agent.

6258. The method of item 6024 wherein the agent is delivered from the implant, wherein the implant comprises about 250 mg to about 1000 mg of the agent.

6259. The method of item 6024 wherein the agent is delivered from the implant, wherein the implant comprises about 1000 mg to about 2500 mg of the agent.

6260. The method of item 6024 wherein the agent is delivered from the implant, wherein a surface of the implant comprises less than 0.01 μg of the agent per mm² of implant surface to which the agent is applied.

6261. The method of item 6024 wherein the agent is delivered from the implant, wherein a surface of the implant comprises about 0.01 μg to about 1 μg of the agent per mm² of implant surface to which the agent is applied.

6262. The method of item 6024 wherein the agent is delivered from the implant, wherein a surface of the implant comprises about 1 μg to about 10 μg of the agent per mm2 of implant surface to which the agent is applied.

6263. The method of item 6024 wherein the agent is delivered from the implant, wherein a surface of the implant comprises about 10 μg to about 250 μg of the agent per mm² of implant surface to which the agent is applied.

6264. The method of item 6024 wherein the agent is delivered from the implant, wherein a surface of the implant comprises about 250 μg to about 1000 μg of the agent per mm² of implant surface to which the agent is applied.

6265. The method of item 6024 wherein the agent is delivered from the implant, wherein a surface of the implant comprises about 1000 μg to about 2500 μg of the agent per mm² of implant surface to which the agent is applied.

6266. The method of item 6024, wherein the implant further comprises a coating, and the coating is a uniform coating.

6267. The method of item 6024, wherein the implant further comprises a coating, and the coating is a non-uniform coating.

6268. The method of item 6024, wherein the implant further comprises a coating, and the coating is a discontinuous coating.

6269. The method of item 6024, wherein the implant further comprises a coating, and the coating is a patterned coating.

6270. The method of item 6024, wherein the implant further comprises a coating, and the coating has a thickness of 100 μm or less.

6271. The method of item 6024, wherein the implant further comprises a coating, and the coating has a thickness of 10 μm or less.

6272. The method of item 6024, wherein the implant further comprises a coating, and the coating adheres to the surface of the implant upon deployment of the implant.

6273. The method of item 6024, wherein the implant further comprises a coating, and the coating is stable at room temperature for a period of at least 1 year.

6274. The method of item 6024, wherein the implant further comprises a coating, and the agent is present in the coating in an amount ranging between about 0.0001% to about 1% by weight.

6275. The method of item 6024, wherein the implant further comprises a coating, and the agent is present in the coating in an amount ranging between about 1% to about 10% by weight.

6276. The method of item 6024, wherein the implant further comprises a coating, and the agent is present in the coating in an amount ranging between about 10% to about 25% by weight.

6277. The method of item 6024, wherein the implant further comprises a coating, and the agent is present in the coating in an amount ranging between about 25% to about 70% by weight.

6278. The method of item 6024, wherein the implant further comprises a coating, and the coating comprises a polymer.

6279. The method of item 6024, wherein the implant comprises a first coating having a first composition and a second coating having a second composition.

6280. The method of item 6024, wherein the implant comprises a first coating having a first composition and a second coating having a second composition, wherein the first composition and the second composition are different.

6281. A method for inhibiting scarring comprising placing a ventricular assist implant and an anti-scarring agent or a composition comprising an anti-scarring agent into an animal host, wherein the agent inhibits scarring.

6282. The method of item 6281 wherein the agent inhibits cell regeneration.

6283. The method of item 6281 wherein the agent inhibits angiogenesis.

6284. The method of item 6281 wherein the agent inhibits fibroblast migration.

6285. The method of item 6281 wherein the agent inhibits fibroblast proliferation.

6286. The method of item 6281 wherein the agent inhibits deposition of extracellular matrix.

6287. The method of item 6281 wherein the agent inhibits tissue remodeling.

6288. The method of item 6281 wherein the agent is an angiogenesis inhibitor.

6289. The method of item 6281 wherein the agent is a 5-lipoxygenase inhibitor or antagonist.

6290. The method of item 6281 wherein the agent is a chemokine receptor antagonist.

6291. The method of item 6281 wherein the agent is a cell cycle inhibitor.

6292. The method of item 6281 wherein the agent is a taxane.

6293. The method of item 6281 wherein the agent is an anti-microtubule agent.

6294. The method of item 6281 wherein the agent is paclitaxel.

6295. The method of item 6281 wherein the agent is not paclitaxel.

6296. The method of item 6281 wherein the agent is an analogue or derivative of paclitaxel.

6297. The method of item 6281 wherein the agent is a vinca alkaloid.

6298. The method of item 6281 wherein the agent is camptothecin or an analogue or derivative thereof.

6299. The method of item 6281 wherein the agent is a podophyllotoxin.

6300. The method of item 6281 wherein the agent is a podophyllotoxin, wherein the podophyllotoxin is etoposide or an analogue or derivative thereof.

6301. The method of item 6281 wherein the agent is an anthracycline.

6302. The method of item 6281 wherein the agent is an anthracycline, wherein the anthracycline is doxorubicin or an analogue or derivative thereof.

6303. The method of item 6281 wherein the agent is an anthracycline, wherein the anthracycline is mitoxantrone or an analogue or derivative thereof.

6304. The method of item 6281 wherein the agent is a platinum compound.

6305. The method of item 6281 wherein the agent is a nitrosourea.

6306. The method of item 6281 wherein the agent is a nitroimidazole.

6307. The method of item 6281 wherein the agent is a folic acid antagonist.

6308. The method of item 6281 wherein the agent is a cytidine analogue.

6309. The method of item 6281 wherein the agent is a pyrimidine analogue.

6310. The method of item 6281 wherein the agent is a fluoropyrimidine analogue.

6311. The method of item 6281 wherein the agent is a purine analogue.

6312. The method of item 6281 wherein the agent is a nitrogen mustard or an analogue or derivative thereof.

6313. The method of item 6281 wherein the agent is a hydroxyurea.

6314. The method of item 6281 wherein the agent is a mytomicin or an analogue or derivative thereof.

6315. The method of item 6281 wherein the agent is an alkyl sulfonate.

6316. The method of item 6281 wherein the agent is a benzamide or an analogue or derivative thereof.

6317. The method of item 6281 wherein the agent is a nicotinamide or an analogue or derivative thereof.

6318. The method of item 6281 wherein the agent is a halogenated sugar or an analogue or derivative thereof.

6319. The method of item 6281 wherein the agent is a DNA alkylating agent.

6320. The method of item 6281 wherein the agent is an anti-microtubule agent.

6321. The method of item 6281 wherein the agent is a topoisomerase inhibitor.

6322. The method of item 6281 wherein the agent is a DNA cleaving agent.

6323. The method of item 6281 wherein the agent is an antimetabolite.

6324. The method of item 6281 wherein the agent inhibits adenosine deaminase.

6325. The method of item 6281 wherein the agent inhibits purine ring synthesis.

6326. The method of item 6281 wherein the agent is a nucleotide interconversion inhibitor.

6327. The method of item 6281 wherein the agent inhibits dihydrofolate reduction.

6328. The method of item 6281 wherein the agent blocks thymidine monophosphate.

6329. The method of item 6281 wherein the agent causes DNA damage.

6330. The method of item 6281 wherein the agent is a DNA intercalation agent.

6331. The method of item 6281 wherein the agent is a RNA synthesis inhibitor.

6332. The method of item 6281 wherein the agent is a pyrimidine synthesis inhibitor.

6333. The method of item 6281 wherein the agent inhibits ribonucleotide synthesis or function.

6334. The method of item 6281 wherein the agent inhibits thymidine monophosphate synthesis or function.

6335. The method of item 6281 wherein the agent inhibits DNA synthesis.

6336. The method of item 6281 wherein the agent causes DNA adduct formation.

6337. The method of item 6281 wherein the agent inhibits protein synthesis.

6338. The method of item 6281 wherein the agent inhibits microtubule function.

6339. The method of item 6281 wherein the agent is a cyclin dependent protein kinase inhibitor.

6340. The method of item 6281 wherein the agent is an epidermal growth factor kinase inhibitor.

6341. The method of item 6281 wherein the agent is an elastase inhibitor.

6342. The method of item 6281 wherein the agent is a factor Xa inhibitor.

6343. The method of item 6281 wherein the agent is a farnesyltransferase inhibitor.

6344. The method of item 6281 wherein the agent is a fibrinogen antagonist.

6345. The method of item 6281 wherein the agent is a guanylate cyclase stimulant.

6346. The method of item 6281 wherein the agent is a heat shock protein 90 antagonist.

6347. The method of item 6281 wherein the agent is a heat shock protein 90 antagonist, wherein the heat shock protein 90 antagonist is geldanamycin or an analogue or derivative thereof.

6348. The method of item 6281 wherein the agent is a guanylate cyclase stimulant.

6349. The method of item 6281 wherein the agent is a HMGCoA reductase inhibitor.

6350. The method of item 6281 wherein the agent is a HMGCoA reductase inhibitor, wherein the HMGCoA reductase inhibitor is simvastatin or an analogue or derivative thereof.

6351. The method of item 6281 wherein the agent is a hydroorotate dehydrogenase inhibitor.

6352. The method of item 6281 wherein the agent is an IKK2 inhibitor.

6353. The method of item 6281 wherein the agent is an IL-1 antagonist.

6354. The method of item 6281 wherein the agent is an ICE antagonist.

6355. The method of item 6281 wherein the agent is an IRAK antagonist.

6356. The method of item 6281 wherein the agent is an IL-4 agonist.

6357. The method of item 6281 wherein the agent is an immunomodulatory agent.

6358. The method of item 6281 wherein the agent is sirolimus or an analogue or derivative thereof.

6359. The method of item 6281 wherein the agent is not sirolimus.

6360. The method of item 6281 wherein the agent is everolimus or an analogue or derivative thereof.

6361. The method of item 6281 wherein the agent is tacrolimus or an analogue or derivative thereof.

6362. The method of item 6281 wherein the agent is not tacrolimus.

6363. The method of item 6281 wherein the agent is biolmus or an analogue or derivative thereof.

6364. The method of item 6281 wherein the agent is tresperimus or an analogue or derivative thereof.

6365. The method of item 6281 wherein the agent is auranofin or an analogue or derivative thereof.

6366. The method of item 6281 wherein the agent is 27-0-demethylrapamycin or an analogue or derivative thereof.

6367. The method of item 6281 wherein the agent is gusperimus or an analogue or derivative thereof.

6368. The method of item 6281 wherein the agent is pimecrolimus or an analogue or derivative thereof.

6369. The method of item 6281 wherein the agent is ABT-578 or an analogue or derivative thereof.

6370. The method of item 6281 wherein the agent is an inosine monophosphate dehydrogenase (IMPDH) inhibitor.

6371. The method of item 6281 wherein the agent is an IMPDH inhibitor, wherein the IMPDH inhibitor is mycophenolic acid or an analogue or derivative thereof.

6372. The method of item 6281 wherein the agent is an IMPDH inhibitor, wherein the IMPDH inhibitor is 1-alpha-25 dihydroxy vitamin D₃ or an analogue or derivative thereof.

6373. The method of item 6281 wherein the agent is a leukotriene inhibitor.

6374. The method of item 6281 wherein the agent is a MCP-1 antagonist.

6375. The method of item 6281 wherein the agent is a MMP inhibitor.

6376. The method of item 6281 wherein the agent is an NF kappa B inhibitor.

6377. The method of item 6281 wherein the agent is an NF kappa B inhibitor, wherein the NF kappa B inhibitor is Bay 11-7082.

6378. The method of item 6281 wherein the agent is an NO agonist.

6379. The method of item 6281 wherein the agent is a p38 MAP kinase inhibitor.

6380. The method of item 6281 wherein the agent is a p38 MAP kinase inhibitor, wherein the p38 MAP kinase inhibitor is SB 202190.

6381. The method of item 6281 wherein the agent is a phosphodiesterase inhibitor.

6382. The method of item 6281 wherein the agent is a TGF beta inhibitor.

6383. The method of item 6281 wherein the agent is a thromboxane A2 antagonist.

6384. The method of item 6281 wherein the agent is a TNFa antagonist.

6385. The method of item 6281 wherein the agent is a TACE inhibitor.

6386. The method of item 6281 wherein the agent is a tyrosine kinase inhibitor.

6387. The method of item 6281 wherein the agent is a vitronectin inhibitor.

6388. The method of item 6281 wherein the agent is a fibroblast growth factor inhibitor.

6389. The method of item 6281 wherein the agent is a protein kinase inhibitor.

6390. The method of item 6281 wherein the agent is a PDGF receptor kinase inhibitor.

6391. The method of item 6281 wherein the agent is an endothelial growth factor receptor kinase inhibitor.

6392. The method of item 6281 wherein the agent is a retinoic acid receptor antagonist.

6393. The method of item 6281 wherein the agent is a platelet derived growth factor receptor kinase inhibitor.

6394. The method of item 6281 wherein the agent is a fibronogin antagonist.

6395. The method of item 6281 wherein the agent is an antimycotic agent.

6396. The method of item 6281 wherein the agent is an antimycotic agent, wherein the antimycotic agent is sulconizole.

6397. The method of item 6281 wherein the agent is a bisphosphonate.

6398. The method of item 6281 wherein the agent is a phospholipase A1 inhibitor.

6399. The method of item 6281 wherein the agent is a histamine H1/H2/H3 receptor antagonist.

6400. The method of item 6281 wherein the agent is a macrolide antibiotic.

6401. The method of item 6281 wherein the agent is a GPIIb/IIIa receptor antagonist.

6402. The method of item 6281 wherein the agent is an endothelin receptor antagonist.

6403. The method of item 6281 wherein the agent is a peroxisome proliferator-activated receptor agonist.

6404. The method of item 6281 wherein the agent is an estrogen receptor agent.

6405. The method of item 6281 wherein the agent is a somastostatin analogue.

6406. The method of item 6281 wherein the agent is a neurokinin 1 antagonist.

6407. The method of item 6281 wherein the agent is a neurokinin 3 antagonist.

6408. The method of item 6281 wherein the agent is a VLA-4 antagonist.

6409. The method of item 6281 wherein the agent is an osteoclast inhibitor.

6410. The method of item 6281 wherein the agent is a DNA topoisomerase ATP hydrolyzing inhibitor.

6411. The method of item 6281 wherein the agent is an angiotensin I converting enzyme inhibitor.

6412. The method of item 6281 wherein the agent is an angiotensin II antagonist.

6413. The method of item 6281 wherein the agent is an enkephalinase inhibitor.

6414. The method of item 6281 wherein the agent is a peroxisome proliferator-activated receptor gamma agonist insulin sensitizer.

6415. The method of item 6281 wherein the agent is a protein kinase C inhibitor.

6416. The method of item 6281 wherein the agent is a ROCK (rho-associated kinase) inhibitor.

6417. The method of item 6281 wherein the agent is a CXCR3 inhibitor.

6418. The method of item 6281 wherein the agent is an Itk inhibitor.

6419. The method of item 6281 wherein the agent is a cytosolic phospholipase A₂-alpha inhibitor.

6420. The method of item 6281 wherein the agent is a PPAR agonist.

6421. The method of item 6281 wherein the agent is an immunosuppressant.

6422. The method of item 6281 wherein the agent is an Erb inhibitor.

6423. The method of item 6281 wherein the agent is an apoptosis agonist.

6424. The method of item 6281 wherein the agent is a lipocortin agonist.

6425. The method of item 6281 wherein the agent is a VCAM-1 antagonist.

6426. The method of item 6281 wherein the agent is a collagen antagonist.

6427. The method of item 6281 wherein the agent is an alpha 2 integrin antagonist.

6428. The method of item 6281 wherein the agent is a TNF alpha inhibitor.

6429. The method of item 6281 wherein the agent is a nitric oxide inhibitor.

6430. The method of item 6281 wherein the agent is a cathepsin inhibitor.

6431. The method of item 6281 wherein the agent is not an anti-inflammatory agent.

6432. The method of item 6281 wherein the agent is not a steroid.

6433. The method of item 6281 wherein the agent is not a glucocorticosteroid.

6434. The method of item 6281 wherein the agent is not dexamethasone.

6435. The method of item 6281 wherein the agent is not an anti-infective agent.

6436. The method of item 6281 wherein the agent is not an antibiotic.

6437. The method of item 6281 wherein the agent is not an anti-fungal agent.

6438. The method of item 6281, wherein the composition comprises a polymer.

6439. The method of item 6281, wherein the composition comprises a polymer, and the polymer is, or comprises, a copolymer.

6440. The method of item 6281, wherein the composition comprises a polymer, and the polymer is, or comprises, a block copolymer.

6441. The method of item 6281, wherein the composition comprises a polymer, and the polymer is, or comprises, a random copolymer.

6442. The method of item 6281, wherein the composition comprises a polymer, and the polymer is, or comprises, a biodegradable polymer.

6443. The method of item 6281, wherein the composition comprises a polymer, and the polymer is, or comprises, a non-biodegradable polymer.

6444. The method of item 6281, wherein the composition comprises a polymer, and the polymer is, or comprises, a hydrophilic polymer.

6445. The method of item 6281, wherein the composition comprises a polymer, and the polymer is, or comprises, a hydrophobic polymer.

6446. The method of item 6281, wherein the composition comprises a polymer, and the polymer is, or comprises, a polymer having hydrophilic domains.

6447. The method of item 6281, wherein the composition comprises a polymer, and the polymer is, or comprises, a polymer having hydrophobic domains.

6448. The method of item 6281, wherein the composition comprises a polymer, and the polymer is, or comprises, a non-conductive polymer.

6449. The method of item 6281, wherein the composition comprises a polymer, and the polymer is, or comprises, an elastomer.

6450. The method of item 6281, wherein the composition comprises a polymer, and the polymer is, or comprises, a hydrogel.

6451. The method of item 6281, wherein the composition comprises a polymer, and the polymer is, or comprises, a silicone polymer.

6452. The method of item 6281, wherein the composition comprises a polymer, and the polymer is, or comprises, a hydrocarbon polymer.

6453. The method of item 6281, wherein the composition comprises a polymer, and the polymer is, or comprises, a styrene-derived polymer.

6454. The method of item 6281, wherein the composition comprises a polymer, and the polymer is, or comprises, a butadiene-derived polymer.

6455. The method of item 6281, wherein the composition comprises a polymer, and the polymer is, or comprises, a macromer.

6456. The method of item 6281, wherein the composition comprises a polymer, and the polymer is, or comprises, a poly(ethylene glycol)polymer.

6457. The method of item 6281, wherein the composition comprises a polymer, and the polymer is, or comprises, an amorphous polymer.

6458. The method of item 6281, wherein the composition further comprises a second pharmaceutically active agent.

6459. The method of item 6281, wherein the composition further comprises an anti-inflammatory agent.

6460. The method of item 6281, wherein the composition further comprises an agent that inhibits infection.

6461. The method of item 6281, wherein the composition further comprises an anthracycline.

6462. The method of item 6281, wherein the composition further comprises doxorubicin.

6463. The method of item 6281 wherein the composition further comprises mitoxantrone.

6464. The method of item 6281 wherein the composition further comprises a fluoropyrimidine.

6465. The method of item 6281, wherein the composition further comprises 5-fluorouracil (5-FU).

6466. The method of item 6281, wherein the composition further comprises a folic acid antagonist.

6467. The method of item 6281, wherein the composition further comprises methotrexate.

6468. The method of item 6281, wherein the composition further comprises a podophylotoxin.

6469. The method of item 6281, wherein the composition further comprises etoposide.

6470. The method of item 6281, wherein the composition further comprises camptothecin.

6471. The method of item 6281, wherein the composition further comprises a hydroxyurea.

6472. The method of item 6281, wherein the composition further comprises a platinum complex.

6473. The method of item 6281, wherein the composition further comprises cisplatin.

6474. The method of item 6281 wherein the composition further comprises an anti-thrombotic agent.

6475. The method of item 6281, wherein the composition further comprises a visualization agent.

6476. The method of item 6281, wherein the composition further comprises a visualization agent, and the visualization agent is a radiopaque material, wherein the radiopaque material comprises a metal, a halogenated compound, or a barium containing compound.

6477. The method of item 6281, wherein the composition further comprises a visualization agent, and the visualization agent is, or comprises, barium, tantalum, or technetium.

6478. The method of item 6281, wherein the composition further comprises a visualization agent, and the visualization agent is, or comprises, an MRI responsive material.

6479. The method of item 6281, wherein the composition further comprises a visualization agent, and the visualization agent is, or comprises, a gadolinium chelate.

6480. The method of item 6281, wherein the composition further comprises a visualization agent, and the visualization agent is, or comprises, iron, magnesium, manganese, copper, or chromium.

6481. The method of item 6281, wherein the composition further comprises a visualization agent, and the visualization agent is, or comprises, iron oxide compound.

6482. The method of item 6281, wherein the composition further comprises a visualization agent, and the visualization agent is, or comprises, a dye, pigment, or colorant.

6483. The method of item 6281 wherein the agent is released in effective concentrations from the composition comprising the agent by diffusion over a period ranging from the time of administration to about 90 days.

6484. The method of item 6281 wherein the agent is released in effective concentrations from the composition comprising the agent by erosion of the composition over a period ranging from the time of administration to about 90 days.

6485. The method of item 6281 wherein the composition further comprises an inflammatory cytokine.

6486. The method of item 6281 wherein the composition further comprises an agent that stimulates cell proliferation.

6487. The method of item 6281 wherein the composition further comprises a polymeric carrier.

6488. The method of item 6281 wherein the composition is in the form of a gel, paste, or spray.

6489. The method of item 6281 wherein the implant is partially constructed with the agent or the composition.

6490. The method of item 6281 wherein the implant is fully constructed with the agent or the composition.

6491. The method of item 6281 wherein the implant is impregnated with the agent or the composition.

6492. The method of item 6281, wherein the agent or the composition forms a coating, and the coating directly contacts the implant.

6493. The method of item 6281, wherein the agent or the composition forms a coating, and the coating indirectly contacts the implant.

6494. The method of item 6281 wherein the agent or the composition forms a coating, and the coating partially covers the implant.

6495. The method of item 6281, wherein the agent or the composition forms a coating, and the coating completely covers the implant.

6496. The method of item 6281 wherein the agent or the composition is located within pores or holes of the implant.

6497. The method of item 6281 wherein the agent or the composition is located within a channel, lumen, or divet of the implant.

6498. The method of item 6281 wherein the implant further comprising an echogenic material.

6499. The method of item 6281 wherein the implant further comprises an echogenic material, wherein the echogenic material is in the form of a coating.

6500. The method of item 6281 wherein the implant is sterile.

6501. The method of item 6281 wherein the agent is delivered from the implant, wherein the agent is released into tissue in the vicinity of the implant after deployment of the implant.

6502. The method of item 6281 wherein the agent is delivered from the implant, wherein the agent is released into tissue in the vicinity of the implant after deployment of the implant, wherein the tissue is connective tissue.

6503. The method of item 6281 wherein the agent is delivered from the implant, wherein the agent is released into tissue in the vicinity of the implant after deployment of the implant, wherein the tissue is muscle tissue.

6504. The method of item 6281 wherein the agent is delivered from the implant, wherein the agent is released into tissue in the vicinity of the implant after deployment of the implant, wherein the tissue is nerve tissue.

6505. The method of item 6281 wherein the agent is delivered from the implant, wherein the agent is released into tissue in the vicinity of the implant after deployment of the implant, wherein the tissue is epithelium tissue.

6506. The method of item 6281 wherein the agent is delivered from the implant, wherein the agent is released in effective concentrations from the implant over a period ranging from the time of deployment of the implant to about 1 year.

6507. The method of item 6281 wherein the agent is delivered from the implant, wherein the agent is released in effective concentrations from the implant over a period ranging from about 1 month to 6 months.

6508. The method of item 6281 wherein the agent is delivered from the implant, wherein the agent is released in effective concentrations from the implant over a period ranging from about 1-90 days.

6509. The method of item 6281 wherein the agent is delivered from the implant, wherein the agent is released in effective concentrations from the implant at a constant rate.

6510. The method of item 6281 wherein the agent is delivered from the implant, wherein the agent is released in effective concentrations from the implant at an increasing rate.

6511. The method of item 6281 wherein the agent is delivered from the implant, wherein the agent is released in effective concentrations from the implant at a decreasing rate.

6512. The method of item 6281 wherein the agent is delivered from the implant, wherein the implant comprises about 0.01 μg to about 10 μg of the agent.

6513. The method of item 6281 wherein the agent is delivered from the implant, wherein the implant comprises about 10 μg to about 10 mg of the agent.

6514. The method of item 6281 wherein the agent is delivered from the implant, wherein the implant comprises about 10 mg to about 250 mg of the agent.

6515. The method of item 6281 wherein the agent is delivered from the implant, wherein the implant comprises about 250 mg to about 1000 mg of the agent.

6516. The method of item 6281 wherein the agent is delivered from the implant, wherein the implant comprises about 1000 mg to about 2500 mg of the agent.

6517. The method of item 6281 wherein the agent is delivered from the implant, wherein a surface of the implant comprises less than 0.01 μg of the agent per mm² of implant surface to which the agent is applied.

6518. The method of item 6281 wherein the agent is delivered from the implant, wherein a surface of the implant comprises about 0.01 μg to about 1 μg of the agent per mm² of implant surface to which the agent is applied.

6519. The method of item 6281 wherein the agent is delivered from the implant, wherein a surface of the implant comprises about 1 μg to about 10 μg of the agent per mm² of implant surface to which the agent is applied.

6520. The method of item 6281 wherein the agent is delivered from the implant, wherein a surface of the implant comprises about 10 μg to about 250 μg of the agent per mm² of implant surface to which the agent is applied.

6521. The method of item 6281 wherein the agent is delivered from the implant, wherein a surface of the implant comprises about 250 μg to about 1000 μg of the agent per mm² of implant surface to which the agent is applied.

6522. The method of item 6281 wherein the agent is delivered from the implant, wherein a surface of the implant comprises about 1000 μg to about 2500 μg of the agent per mm² of implant surface to which the agent is applied.

6523. The method of item 6281, wherein the implant further comprises a coating, and the coating is a uniform coating.

6524. The method of item 6281, wherein the implant further comprises a coating, and the coating is a non-uniform coating.

6525. The method of item 6281, wherein the implant further comprises a coating, and the coating is a discontinuous coating.

6526. The method of item 6281, wherein the implant further comprises a coating, and the coating is a patterned coating.

6527. The method of item 6281, wherein the implant further comprises a coating, and the coating has a thickness of 100 μm or less.

6528. The method of item 6281, wherein the implant further comprises a coating, and the coating has a thickness of 10 μm or less.

6529. The method of item 6281, wherein the implant further comprises a coating, and the coating adheres to the surface of the implant upon deployment of the implant.

6530. The method of item 6281, wherein the implant further comprises a coating, and the coating is stable at room temperature for a period of at least 1 year.

6531. The method of item 6281, wherein the implant further comprises a coating, and the agent is present in the coating in an amount ranging between about 0.0001% to about 1% by weight.

6532. The method of item 6281, wherein the implant further comprises a coating, and the agent is present in the coating in an amount ranging between about 1% to about 10% by weight.

6533. The method of item 6281, wherein the implant further comprises a coating, and the agent is present in the coating in an amount ranging between about 10% to about 25% by weight.

6534. The method of item 6281, wherein the implant further comprises a coating, and the agent is present in the coating in an amount ranging between about 25% to about 70% by weight.

6535. The method of item 6281, wherein the implant further comprises a coating, and the coating comprises a polymer.

6536. The method of item 6281, wherein the implant comprises a first coating having a first composition and a second coating having a second composition.

6537. The method of item 6281, wherein the implant comprises a first coating having a first composition and a second coating having a second composition, wherein the first composition and the second composition are different.

6538. A method for inhibiting scarring comprising placing a prosthetic heart valve implant and an anti-scarring agent or a composition comprising an anti-scarring agent into an animal host, wherein the agent inhibits scarring.

6539. The method of item 6538 wherein the agent inhibits cell regeneration.

6540. The method of item 6538 wherein the agent inhibits angiogenesis.

6541. The method of item 6538 wherein the agent inhibits fibroblast migration.

6542. The method of item 6538 wherein the agent inhibits fibroblast proliferation.

6543. The method of item 6538 wherein the agent inhibits deposition of extracellular matrix.

6544. The method of item 6538 wherein the agent inhibits tissue remodeling.

6545. The method of item 6538 wherein the agent is an angiogenesis inhibitor.

6546. The method of item 6538 wherein the agent is a 5-lipoxygenase inhibitor or antagonist.

6547. The method of item 6538 wherein the agent is a chemokine receptor antagonist.

6548. The method of item 6538 wherein the agent is a cell cycle inhibitor.

6549. The method of item 6538 wherein the agent is a taxane.

6550. The method of item 6538 wherein the agent is an anti-microtubule agent.

6551. The method of item 6538 wherein the agent is paclitaxel.

6552. The method of item 6538 wherein the agent is not paclitaxel.

6553. The method of item 6538 wherein the agent is an analogue or derivative of paclitaxel.

6554. The method of item 6538 wherein the agent is a vinca alkaloid.

6555. The method of item 6538 wherein the agent is camptothecin or an analogue or derivative thereof.

6556. The method of item 6538 wherein the agent is a podophyllotoxin.

6557. The method of item 6538 wherein the agent is a podophyllotoxin, wherein the podophyllotoxin is etoposide or an analogue or derivative thereof.

6558. The method of item 6538 wherein the agent is an anthracycline.

6559. The method of item 6538 wherein the agent is an anthracycline, wherein the anthracycline is doxorubicin or an analogue or derivative thereof.

6560. The method of item 6538 wherein the agent is an anthracycline, wherein the anthracycline is mitoxantrone or an analogue or derivative thereof.

6561. The method of item 6538 wherein the agent is a platinum compound.

6562. The method of item 6538 wherein the agent is a nitrosourea.

6563. The method of item 6538 wherein the agent is a nitroimidazole.

6564. The method of item 6538 wherein the agent is a folic acid antagonist.

6565. The method of item 6538 wherein the agent is a cytidine analogue.

6566. The method of item 6538 wherein the agent is a pyrimidine analogue.

6567. The method of item 6538 wherein the agent is a fluoropyrimidine analogue.

6568. The method of item 6538 wherein the agent is a purine analogue.

6569. The method of item 6538 wherein the agent is a nitrogen mustard or an analogue or derivative thereof.

6570. The method of item 6538 wherein the agent is a hydroxyurea.

6571. The method of item 6538 wherein the agent is a mytomicin or an analogue or derivative thereof.

6572. The method of item 6538 wherein the agent is an alkyl sulfonate.

6573. The method of item 6538 wherein the agent is a benzamide or an analogue or derivative thereof.

6574. The method of item 6538 wherein the agent is a nicotinamide or an analogue or derivative thereof.

6575. The method of item 6538 wherein the agent is a halogenated sugar or an analogue or derivative thereof.

6576. The method of item 6538 wherein the agent is a DNA alkylating agent.

6577. The method of item 6538 wherein the agent is an anti-microtubule agent.

6578. The method of item 6538 wherein the agent is a topoisomerase inhibitor.

6579. The method of item 6538 wherein the agent is a DNA cleaving agent.

6580. The method of item 6538 wherein the agent is an antimetabolite.

6581. The method of item 6538 wherein the agent inhibits adenosine deaminase.

6582. The method of item 6538 wherein the agent inhibits purine ring synthesis.

6583. The method of item 6538 wherein the agent is a nucleotide interconversion inhibitor.

6584. The method of item 6538 wherein the agent inhibits dihydrofolate reduction.

6585. The method of item 6538 wherein the agent blocks thymidine monophosphate.

6586. The method of item 6538 wherein the agent causes DNA damage.

6587. The method of item 6538 wherein the agent is a DNA intercalation agent.

6588. The method of item 6538 wherein the agent is a RNA synthesis inhibitor.

6589. The method of item 6538 wherein the agent is a pyrimidine synthesis inhibitor.

6590. The method of item 6538 wherein the agent inhibits ribonucleotide synthesis or function.

6591. The method of item 6538 wherein the agent inhibits thymidine monophosphate synthesis or function.

6592. The method of item 6538 wherein the agent inhibits DNA synthesis.

6593. The method of item 6538 wherein the agent causes DNA adduct formation.

6594. The method of item 6538 wherein the agent inhibits protein synthesis.

6595. The method of item 6538 wherein the agent inhibits microtubule function.

6596. The method of item 6538 wherein the agent is a cyclin dependent protein kinase inhibitor.

6597. The method of item 6538 wherein the agent is an epidermal growth factor kinase inhibitor.

6598. The method of item 6538 wherein the agent is an elastase inhibitor.

6599. The method of item 6538 wherein the agent is a factor Xa inhibitor.

6600. The method of item 6538 wherein the agent is a farnesyltransferase inhibitor.

6601. The method of item 6538 wherein the agent is a fibrinogen antagonist.

6602. The method of item 6538 wherein the agent is a guanylate cyclase stimulant.

6603. The method of item 6538 wherein the agent is a heat shock protein 90 antagonist.

6604. The method of item 6538 wherein the agent is a heat shock protein 90 antagonist, wherein the heat shock protein 90 antagonist is geldanamycin or an analogue or derivative thereof.

6605. The method of item 6538 wherein the agent is a guanylate cyclase stimulant.

6606. The method of item 6538 wherein the agent is a HMGCoA reductase inhibitor.

6607. The method of item 6538 wherein the agent is a HMGCoA reductase inhibitor, wherein the HMGCoA reductase inhibitor is simvastatin or an analogue or derivative thereof.

6608. The method of item 6538 wherein the agent is a hydroorotate dehydrogenase inhibitor.

6609. The method of item 6538 wherein the agent is an IKK2 inhibitor.

6610. The method of item 6538 wherein the agent is an IL-1 antagonist.

6611. The method of item 6538 wherein the agent is an ICE antagonist.

6612. The method of item 6538 wherein the agent is an IRAK antagonist.

6613. The method of item 6538 wherein the agent is an IL-4 agonist.

6614. The method of item 6538 wherein the agent is an immunomodulatory agent.

6615. The method of item 6538 wherein the agent is sirolimus or an analogue or derivative thereof.

6616. The method of item 6538 wherein the agent is not sirolimus.

6617. The method of item 6538 wherein the agent is everolimus or an analogue or derivative thereof.

6618. The method of item 6538 wherein the agent is tacrolimus or an analogue or derivative thereof.

6619. The method of item 6538 wherein the agent is not tacrolimus.

6620. The method of item 6538 wherein the agent is biolmus or an analogue or derivative thereof.

6621. The method of item 6538 wherein the agent is tresperimus or an analogue or derivative thereof.

6622. The method of item 6538 wherein the agent is auranofin or an analogue or derivative thereof.

6623. The method of item 6538 wherein the agent is 27-0-demethylrapamycin or an analogue or derivative thereof.

6624. The method of item 6538 wherein the agent is gusperimus or an analogue or derivative thereof.

6625. The method of item 6538 wherein the agent is pimecrolimus or an analogue or derivative thereof.

6626. The method of item 6538 wherein the agent is ABT-578 or an analogue or derivative thereof.

6627. The method of item 6538 wherein the agent is an inosine monophosphate dehydrogenase (IMPDH) inhibitor.

6628. The method of item 6538 wherein the agent is an IMPDH inhibitor, wherein the IMPDH inhibitor is mycophenolic acid or an analogue or derivative thereof.

6629. The method of item 6538 wherein the agent is an IMPDH inhibitor, wherein the IMPDH inhibitor is 1-alpha-25 dihydroxy vitamin D₃ or an analogue or derivative thereof.

6630. The method of item 6538 wherein the agent is a leukotriene inhibitor.

6631. The method of item 6538 wherein the agent is a MCP-1 antagonist.

6632. The method of item 6538 wherein the agent is a MMP inhibitor.

6633. The method of item 6538 wherein the agent is an NF kappa B inhibitor.

6634. The method of item 6538 wherein the agent is an NF kappa B inhibitor, wherein the NF kappa B inhibitor is Bay 11-7082.

6635. The method of item 6538 wherein the agent is an NO agonist.

6636. The method of item 6538 wherein the agent is a p38 MAP kinase inhibitor.

6637. The method of item 6538 wherein the agent is a p38 MAP kinase inhibitor, wherein the p38 MAP kinase inhibitor is SB 202190.

6638. The method of item 6538 wherein the agent is a phosphodiesterase inhibitor.

6639. The method of item 6538 wherein the agent is a TGF beta inhibitor.

6640. The method of item 6538 wherein the agent is a thromboxane A2 antagonist.

6641. The method of item 6538 wherein the agent is a TNFa antagonist.

6642. The method of item 6538 wherein the agent is a TACE inhibitor.

6643. The method of item 6538 wherein the agent is a tyrosine kinase inhibitor.

6644. The method of item 6538 wherein the agent is a vitronectin inhibitor.

6645. The method of item 6538 wherein the agent is a fibroblast growth factor inhibitor.

6646. The method of item 6538 wherein the agent is a protein kinase inhibitor.

6647. The method of item 6538 wherein the agent is a PDGF receptor kinase inhibitor.

6648. The method of item 6538 wherein the agent is an endothelial growth factor receptor kinase inhibitor.

6649. The method of item 6538 wherein the agent is a retinoic acid receptor antagonist.

6650. The method of item 6538 wherein the agent is a platelet derived growth factor receptor kinase inhibitor.

6651. The method of item 6538 wherein the agent is a fibronogin antagonist.

6652. The method of item 6538 wherein the agent is an antimycotic agent.

6653. The method of item 6538 wherein the agent is an antimycotic agent, wherein the antimycotic agent is sulconizole.

6654. The method of item 6538 wherein the agent is a bisphosphonate.

6655. The method of item 6538 wherein the agent is a phospholipase A1 inhibitor.

6656. The method of item 6538 wherein the agent is a histamine H1/H2/H3 receptor antagonist.

6657. The method of item 6538 wherein the agent is a macrolide antibiotic.

6658. The method of item 6538 wherein the agent is a GPIIb/IIIa receptor antagonist.

6659. The method of item 6538 wherein the agent is an endothelin receptor antagonist.

6660. The method of item 6538 wherein the agent is a peroxisome proliferator-activated receptor agonist.

6661. The method of item 6538 wherein the agent is an estrogen receptor agent.

6662. The method of item 6538 wherein the agent is a somastostatin analogue.

6663. The method of item 6538 wherein the agent is a neurokinin 1 antagonist.

6664. The method of item 6538 wherein the agent is a neurokinin 3 antagonist.

6665. The method of item 6538 wherein the agent is a VLA-4 antagonist.

6666. The method of item 6538 wherein the agent is an osteoclast inhibitor.

6667. The method of item 6538 wherein the agent is a DNA topoisomerase ATP hydrolyzing inhibitor.

6668. The method of item 6538 wherein the agent is an angiotensin I converting enzyme inhibitor.

6669. The method of item 6538 wherein the agent is an angiotensin II antagonist.

6670. The method of item 6538 wherein the agent is an enkephalinase inhibitor.

6671. The method of item 6538 wherein the agent is a peroxisome proliferator-activated receptor gamma agonist insulin sensitizer.

6672. The method of item 6538 wherein the agent is a protein kinase C inhibitor.

6673. The method of item 6538 wherein the agent is a ROCK (rho-associated kinase) inhibitor.

6674. The method of item 6538 wherein the agent is a CXCR3 inhibitor.

6675. The method of item 6538 wherein the agent is an Itk inhibitor.

6676. The method of item 6538 wherein the agent is a cytosolic phospholipase A₂-alpha inhibitor.

6677. The method of item 6538 wherein the agent is a PPAR agonist.

6678. The method of item 6538 wherein the agent is an immunosuppressant.

6679. The method of item 6538 wherein the agent is an Erb inhibitor.

6680. The method of item 6538 wherein the agent is an apoptosis agonist.

6681. The method of item 6538 wherein the agent is a lipocortin agonist.

6682. The method of item 6538 wherein the agent is a VCAM-1 antagonist.

6683. The method of item 6538 wherein the agent is a collagen antagonist.

6684. The method of item 6538 wherein the agent is an alpha 2 integrin antagonist.

6685. The method of item 6538 wherein the agent is a TNF alpha inhibitor.

6686. The method of item 6538 wherein the agent is a nitric oxide inhibitor.

6687. The method of item 6538 wherein the agent is a cathepsin inhibitor.

6688. The method of item 6538 wherein the agent is not an anti-inflammatory agent.

6689. The method of item 6538 wherein the agent is not a steroid.

6690. The method of item 6538 wherein the agent is not a glucocorticosteroid.

6691. The method of item 6538 wherein the agent is not dexamethasone.

6692. The method of item 6538 wherein the agent is not an anti- infective agent.

6693. The method of item 6538 wherein the agent is not an antibiotic.

6694. The method of item 6538 wherein the agent is not an anti- fungal agent.

6695. The method of item 6538, wherein the composition comprises a polymer.

6696. The method of item 6538, wherein the composition comprises a polymer, and the polymer is, or comprises, a copolymer.

6697. The method of item 6538, wherein the composition comprises a polymer, and the polymer is, or comprises, a block copolymer.

6698. The method of item 6538, wherein the composition comprises a polymer, and the polymer is, or comprises, a random copolymer.

6699. The method of item 6538, wherein the composition comprises a polymer, and the polymer is, or comprises, a biodegradable polymer.

6700. The method of item 6538, wherein the composition comprises a polymer, and the polymer is, or comprises, a non-biodegradable polymer.

6701. The method of item 6538, wherein the composition comprises a polymer, and the polymer is, or comprises, a hydrophilic polymer.

6702. The method of item 6538, wherein the composition comprises a polymer, and the polymer is, or comprises, a hydrophobic polymer.

6703. The method of item 6538, wherein the composition comprises a polymer, and the polymer is, or comprises, a polymer having hydrophilic domains.

6704. The method of item 6538, wherein the composition comprises a polymer, and the polymer is, or comprises, a polymer having hydrophobic domains.

6705. The method of item 6538, wherein the composition comprises a polymer, and the polymer is, or comprises, a non-conductive polymer.

6706. The method of item 6538, wherein the composition comprises a polymer, and the polymer is, or comprises, an elastomer.

6707. The method of item 6538, wherein the composition comprises a polymer, and the polymer is, or comprises, a hydrogel.

6708. The method of item 6538, wherein the composition comprises a polymer, and the polymer is, or comprises, a silicone polymer.

6709. The method of item 6538, wherein the composition comprises a polymer, and the polymer is, or comprises, a hydrocarbon polymer.

6710. The method of item 6538, wherein the composition comprises a polymer, and the polymer is, or comprises, a styrene-derived polymer.

6711. The method of item 6538, wherein the composition comprises a polymer, and the polymer is, or comprises, a butadiene-derived polymer.

6712. The method of item 6538, wherein the composition comprises a polymer, and the polymer is, or comprises, a macromer.

6713. The method of item 6538, wherein the composition comprises a polymer, and the polymer is, or comprises, a poly(ethylene glycol)polymer.

6714. The method of item 6538, wherein the composition comprises a polymer, and the polymer is, or comprises, an amorphous polymer.

6715. The method of item 6538, wherein the composition further comprises a second pharmaceutically active agent.

6716. The method of item 6538, wherein the composition further comprises an anti-inflammatory agent.

6717. The method of item 6538, wherein the composition further comprises an agent that inhibits infection.

6718. The method of item 6538, wherein the composition further comprises an anthracycline.

6719. The method of item 6538, wherein the composition further comprises doxorubicin.

6720. The method of item 6538 wherein the composition further comprises mitoxantrone.

6721. The method of item 6538 wherein the composition further comprises a fluoropyrimidine.

6722. The method of item 6538, wherein the composition further comprises 5-fluorouracil (5-FU).

6723. The method of item 6538, wherein the composition further comprises a folic acid antagonist.

6724. The method of item 6538, wherein the composition further comprises methotrexate.

6725. The method of item 6538, wherein the composition further comprises a podophylotoxin.

6726. The method of item 6538, wherein the composition further comprises etoposide.

6727. The method of item 6538, wherein the composition further comprises camptothecin.

6728. The method of item 6538, wherein the composition further comprises a hydroxyurea.

6729. The method of item 6538, wherein the composition further comprises a platinum complex.

6730. The method of item 6538, wherein the composition further comprises cisplatin.

6731. The method of item 6538 wherein the composition further comprises an anti-thrombotic agent.

6732. The method of item 6538, wherein the composition further comprises a visualization agent.

6733. The method of item 6538, wherein the composition further comprises a visualization agent, and the visualization agent is a radiopaque material, wherein the radiopaque material comprises a metal, a halogenated compound, or a barium containing compound.

6734. The method of item 6538, wherein the composition further comprises a visualization agent, and the visualization agent is, or comprises, barium, tantalum, or technetium.

6735. The method of item 6538, wherein the composition further comprises a visualization agent, and the visualization agent is, or comprises, an MRI responsive material.

6736. The method of item 6538, wherein the composition further comprises a visualization agent, and the visualization agent is, or comprises, a gadolinium chelate.

6737. The method of item 6538, wherein the composition further comprises a visualization agent, and the visualization agent is, or comprises, iron, magnesium, manganese, copper, or chromium.

6738. The method of item 6538, wherein the composition further comprises a visualization agent, and the visualization agent is, or comprises, iron oxide compound.

6739. The method of item 6538, wherein the composition further comprises a visualization agent, and the visualization agent is, or comprises, a dye, pigment, or colorant.

6740. The method of item 6538 wherein the agent is released in effective concentrations from the composition comprising the agent by diffusion over a period ranging from the time of administration to about 90 days.

6741. The method of item 6538 wherein the agent is released in effective concentrations from the composition comprising the agent by erosion of the composition over a period ranging from the time of administration to about 90 days.

6742. The method of item 6538 wherein the composition further comprises an inflammatory cytokine.

6743. The method of item 6538 wherein the composition further comprises an agent that stimulates cell proliferation.

6744. The method of item 6538 wherein the composition further comprises a polymeric carrier.

6745. The method of item 6538 wherein the composition is in the form of a gel, paste, or spray.

6746. The method of item 6538 wherein the implant is partially constructed with the agent or the composition.

6747. The method of item 6538 wherein the implant is fully constructed with the agent or the composition.

6748. The method of item 6538 wherein the implant is impregnated with the agent or the composition.

6749. The method of item 6538, wherein the agent or the composition forms a coating, and the coating directly contacts the implant.

6750. The method of item 6538, wherein the agent or the composition forms a coating, and the coating indirectly contacts the implant.

6751. The method of item 6538 wherein the agent or the composition forms a coating, and the coating partially covers the implant.

6752. The method of item 6538, wherein the agent or the composition forms a coating, and the coating completely covers the implant.

6753. The method of item 6538 wherein the agent or the composition is located within pores or holes of the implant.

6754. The method of item 6538 wherein the agent or the composition is located within a channel, lumen, or divet of the implant.

6755. The method of item 6538 wherein the implant further comprising an echogenic material.

6756. The method of item 6538 wherein the implant further comprises an echogenic material, wherein the echogenic material is in the form of a coating.

6757. The method of item 6538 wherein the implant is sterile.

6758. The method of item 6538 wherein the agent is delivered from the implant, wherein the agent is released into tissue in the vicinity of the implant after deployment of the implant.

6759. The method of item 6538 wherein the agent is delivered from the implant, wherein the agent is released into tissue in the vicinity of the implant after deployment of the implant, wherein the tissue is connective tissue.

6760. The method of item 6538 wherein the agent is delivered from the implant, wherein the agent is released into tissue in the vicinity of the implant after deployment of the implant, wherein the tissue is muscle tissue.

6761. The method of item 6538 wherein the agent is delivered from the implant, wherein the agent is released into tissue in the vicinity of the implant after deployment of the implant, wherein the tissue is nerve tissue.

6762. The method of item 6538 wherein the agent is delivered from the implant, wherein the agent is released into tissue in the vicinity of the implant after deployment of the implant, wherein the tissue is epithelium tissue.

6763. The method of item 6538 wherein the agent is delivered from the implant, wherein the agent is released in effective concentrations from the implant over a period ranging from the time of deployment of the implant to about 1 year.

6764. The method of item 6538 wherein the agent is delivered from the implant, wherein the agent is released in effective concentrations from the implant over a period ranging from about 1 month to 6 months.

6765. The method of item 6538 wherein the agent is delivered from the implant, wherein the agent is released in effective concentrations from the implant over a period ranging from about 1-90 days.

6766. The method of item 6538 wherein the agent is delivered from the implant, wherein the agent is released in effective concentrations from the implant at a constant rate.

6767. The method of item 6538 wherein the agent is delivered from the implant, wherein the agent is released in effective concentrations from the implant at an increasing rate.

6768. The method of item 6538 wherein the agent is delivered from the implant, wherein the agent is released in effective concentrations from the implant at a decreasing rate.

6769. The method of item 6538 wherein the agent is delivered from the implant, wherein the implant comprises about 0.01 μg to about 10 μg of the agent.

6770. The method of item 6538 wherein the agent is delivered from the implant, wherein the implant comprises about 10 μg to about 10 mg of the agent.

6771. The method of item 6538 wherein the agent is delivered from the implant, wherein the implant comprises about 10 mg to about 250 mg of the agent.

6772. The method of item 6538 wherein the agent is delivered from the implant, wherein the implant comprises about 250 mg to about 1000 mg of the agent.

6773. The method of item 6538 wherein the agent is delivered from the implant, wherein the implant comprises about 1000 mg to about 2500 mg of the agent.

6774. The method of item 6538 wherein the agent is delivered from the implant, wherein a surface of the implant comprises less than 0.01 μg of the agent per mm2 of implant surface to which the agent is applied.

6775. The method of item 6538 wherein the agent is delivered from the implant, wherein a surface of the implant comprises about 0.01 μg to about 1 μg of the agent per mm² of implant surface to which the agent is applied.

6776. The method of item 6538 wherein the agent is delivered from the implant, wherein a surface of the implant comprises about 1 μg to about 10 μg of the agent per mm² of implant surface to which the agent is applied.

6777. The method of item 6538 wherein the agent is delivered from the implant, wherein a surface of the implant comprises about 10 μg to about 250 μg of the agent per mm² of implant surface to which the agent is applied.

6778. The method of item 6538 wherein the agent is delivered from the implant, wherein a surface of the implant comprises about 250 μg to about 1000 μg of the agent per mm² of implant surface to which the agent is applied.

6779. The method of item 6538 wherein the agent is delivered from the implant, wherein a surface of the implant comprises about 1000 μg to about 2500 μg of the agent per mm² of implant surface to which the agent is applied.

6780. The method of item 6538, wherein the implant further comprises a coating, and the coating is a uniform coating.

6781. The method of item 6538, wherein the implant further comprises a coating, and the coating is a non-uniform coating.

6782. The method of item 6538, wherein the implant further comprises a coating, and the coating is a discontinuous coating.

6783. The method of item 6538, wherein the implant further comprises a coating, and the coating is a patterned coating.

6784. The method of item 6538, wherein the implant further comprises a coating, and the coating has a thickness of 100 μm or less.

6785. The method of item 6538, wherein the implant further comprises a coating, and the coating has a thickness of 10 μm or less.

6786. The method of item 6538, wherein the implant further comprises a coating, and the coating adheres to the surface of the implant upon deployment of the implant.

6787. The method of item 6538, wherein the implant further comprises a coating, and the coating is stable at room temperature for a period of at least 1 year.

6788. The method of item 6538, wherein the implant further comprises a coating, and the agent is present in the coating in an amount ranging between about 0.0001% to about 1% by weight.

6789. The method of item 6538, wherein the implant further comprises a coating, and the agent is present in the coating in an amount ranging between about 1% to about 10% by weight.

6790. The method of item 6538, wherein the implant further comprises a coating, and the agent is present in the coating in an amount ranging between about 10% to about 25% by weight.

6791. The method of item 6538, wherein the implant further comprises a coating, and the agent is present in the coating in an amount ranging between about 25% to about 70% by weight.

6792. The method of item 6538, wherein the implant further comprises a coating, and the coating comprises a polymer.

6793. The method of item 6538, wherein the implant comprises a first coating having a first composition and a second coating having a second composition.

6794. The method of item 6538, wherein the implant comprises a first coating having a first composition and a second coating having a second composition, wherein the first composition and the second composition are different.

6795. A method for inhibiting scarring comprising placing an inferior vena cava filter implant and an anti-scarring agent or a composition comprising an anti-scarring agent into an animal host, wherein the agent inhibits scarring.

6796. The method of item 6795 wherein the agent inhibits cell regeneration.

6797. The method of item 6795 wherein the agent inhibits angiogenesis.

6798. The method of item 6795 wherein the agent inhibits fibroblast migration.

6799. The method of item 6795 wherein the agent inhibits fibroblast proliferation.

6800. The method of item 6795 wherein the agent inhibits deposition of extracellular matrix.

6801. The method of item 6795 wherein the agent inhibits tissue remodeling.

6802. The method of item 6795 wherein the agent is an angiogenesis inhibitor.

6803. The method of item 6795 wherein the agent is a 5-lipoxygenase inhibitor or antagonist.

6804. The method of item 6795 wherein the agent is a chemokine receptor antagonist.

6805. The method of item 6795 wherein the agent is a cell cycle inhibitor.

6806. The method of item 6795 wherein the agent is a taxane.

6807. The method of item 6795 wherein the agent is an anti-microtubule agent.

6808. The method of item 6795 wherein the agent is paclitaxel.

6809. The method of item 6795 wherein the agent is not paclitaxel.

6810. The method of item 6795 wherein the agent is an analogue or derivative of paclitaxel.

6811. The method of item 6795 wherein the agent is a vinca alkaloid.

6812. The method of item 6795 wherein the agent is camptothecin or an analogue or derivative thereof.

6813. The method of item 6795 wherein the agent is a podophyllotoxin.

6814. The method of item 6795 wherein the agent is a podophyllotoxin, wherein the podophyllotoxin is etoposide or an analogue or derivative thereof.

6815. The method of item 6795 wherein the agent is an anthracycline.

6816. The method of item 6795 wherein the agent is an anthracycline, wherein the anthracycline is doxorubicin or an analogue or derivative thereof.

6817. The method of item 6795 wherein the agent is an anthracycline, wherein the anthracycline is mitoxantrone or an analogue or derivative thereof.

6818. The method of item 6795 wherein the agent is a platinum compound.

6819. The method of item 6795 wherein the agent is a nitrosourea.

6820. The method of item 6795 wherein the agent is a nitroimidazole.

6821. The method of item 6795 wherein the agent is a folic acid antagonist.

6822. The method of item 6795 wherein the agent is a cytidine analogue.

6823. The method of item 6795 wherein the agent is a pyrimidine analogue.

6824. The method of item 6795 wherein the agent is a fluoropyrimidine analogue.

6825. The method of item 6795 wherein the agent is a purine analogue.

6826. The method of item 6795 wherein the agent is a nitrogen mustard or an analogue or derivative thereof.

6827. The method of item 6795 wherein the agent is a hydroxyurea.

6828. The method of item 6795 wherein the agent is a mytomicin or an analogue or derivative thereof.

6829. The method of item 6795 wherein the agent is an alkyl sulfonate.

6830. The method of item 6795 wherein the agent is a benzamide or an analogue or derivative thereof.

6831. The method of item 6795 wherein the agent is a nicotinamide or an analogue or derivative thereof.

6832. The method of item 6795 wherein the agent is a halogenated sugar or an analogue or derivative thereof.

6833. The method of item 6795 wherein the agent is a DNA alkylating agent.

6834. The method of item 6795 wherein the agent is an anti-microtubule agent.

6835. The method of item 6795 wherein the agent is a topoisomerase inhibitor.

6836. The method of item 6795 wherein the agent is a DNA cleaving agent.

6837. The method of item 6795 wherein the agent is an antimetabolite.

6838. The method of item 6795 wherein the agent inhibits adenosine deaminase.

6839. The method of item 6795 wherein the agent inhibits purine ring synthesis.

6840. The method of item 6795 wherein the agent is a nucleotide interconversion inhibitor.

6841. The method of item 6795 wherein the agent inhibits dihydrofolate reduction.

6842. The method of item 6795 wherein the agent blocks thymidine monophosphate.

6843. The method of item 6795 wherein the agent causes DNA damage.

6844. The method of item 6795 wherein the agent is a DNA intercalation agent.

6845. The method of item 6795 wherein the agent is a RNA synthesis inhibitor.

6846. The method of item 6795 wherein the agent is a pyrimidine synthesis inhibitor.

6847. The method of item 6795 wherein the agent inhibits ribonucleotide synthesis or function.

6848. The method of item 6795 wherein the agent inhibits thymidine monophosphate synthesis or function.

6849. The method of item 6795 wherein the agent inhibits DNA synthesis.

6850. The method of item 6795 wherein the agent causes DNA adduct formation.

6851. The method of item 6795 wherein the agent inhibits protein synthesis.

6852. The method of item 6795 wherein the agent inhibits microtubule function.

6853. The method of item 6795 wherein the agent is a cyclin dependent protein kinase inhibitor.

6854. The method of item 6795 wherein the agent is an epidermal growth factor kinase inhibitor.

6855. The method of item 6795 wherein the agent is an elastase inhibitor.

6856. The method of item 6795 wherein the agent is a factor Xa inhibitor.

6857. The method of item 6795 wherein the agent is a farnesyltransferase inhibitor.

6858. The method of item 6795 wherein the agent is a fibrinogen antagonist.

6859. The method of item 6795 wherein the agent is a guanylate cyclase stimulant.

6860. The method of item 6795 wherein the agent is a heat shock protein 90 antagonist.

6861. The method of item 6795 wherein the agent is a heat shock protein 90 antagonist, wherein the heat shock protein 90 antagonist is geldanamycin or an analogue or derivative thereof.

6862. The method of item 6795 wherein the agent is a guanylate cyclase stimulant.

6863. The method of item 6795 wherein the agent is a HMGCoA reductase inhibitor.

6864. The method of item 6795 wherein the agent is a HMGCoA reductase inhibitor, wherein the HMGCoA reductase inhibitor is simvastatin or an analogue or derivative thereof.

6865. The method of item 6795 wherein the agent is a hydroorotate dehydrogenase inhibitor.

6866. The method of item 6795 wherein the agent is an IKK2 inhibitor.

6867. The method of item 6795 wherein the agent is an IL-1 antagonist.

6868. The method of item 6795 wherein the agent is an ICE antagonist.

6869. The method of item 6795 wherein the agent is an IRAK antagonist.

6870. The method of item 6795 wherein the agent is an IL-4 agonist.

6871. The method of item 6795 wherein the agent is an immunomodulatory agent.

6872. The method of item 6795 wherein the agent is sirolimus or an analogue or derivative thereof.

6873. The method of item 6795 wherein the agent is not sirolimus.

6874. The method of item 6795 wherein the agent is everolimus or an analogue or derivative thereof.

6875. The method of item 6795 wherein the agent is tacrolimus or an analogue or derivative thereof.

6876. The method of item 6795 wherein the agent is not tacrolimus.

6877. The method of item 6795 wherein the agent is biolmus or an analogue or derivative thereof.

6878. The method of item 6795 wherein the agent is tresperimus or an analogue or derivative thereof.

6879. The method of item 6795 wherein the agent is auranofin or an analogue or derivative thereof.

6880. The method of item 6795 wherein the agent is 27-0-demethylrapamycin or an analogue or derivative thereof.

6881. The method of item 6795 wherein the agent is gusperimus or an analogue or derivative thereof.

6882. The method of item 6795 wherein the agent is pimecrolimus or an analogue or derivative thereof.

6883. The method of item 6795 wherein the agent is ABT-578 or an analogue or derivative thereof.

6884. The method of item 6795 wherein the agent is an inosine monophosphate dehydrogenase (IMPDH) inhibitor.

6885. The method of item 6795 wherein the agent is an IMPDH inhibitor, wherein the IMPDH inhibitor is mycophenolic acid or an analogue or derivative thereof.

6886. The method of item 6795 wherein the agent is an IMPDH inhibitor, wherein the IMPDH inhibitor is 1-alpha-25 dihydroxy vitamin D₃ or an analogue or derivative thereof.

6887. The method of item 6795 wherein the agent is a leukotriene inhibitor.

6888. The method of item 6795 wherein the agent is a MCP-1 antagonist.

6889. The method of item 6795 wherein the agent is a MMP inhibitor.

6890. The method of item 6795 wherein the agent is an NF kappa B inhibitor.

6891. The method of item 6795 wherein the agent is an NF kappa B inhibitor, wherein the NF kappa B inhibitor is Bay 11-7082.

6892. The method of item 6795 wherein the agent is an NO agonist.

6893. The method of item 6795 wherein the agent is a p38 MAP kinase inhibitor.

6894. The method of item 6795 wherein the agent is a p38 MAP kinase inhibitor, wherein the p38 MAP kinase inhibitor is SB 202190.

6895. The method of item 6795 wherein the agent is a phosphodiesterase inhibitor.

6896. The method of item 6795 wherein the agent is a TGF beta inhibitor.

6897. The method of item 6795 wherein the agent is a thromboxane A2 antagonist.

6898. The method of item 6795 wherein the agent is a TNFa antagonist.

6899. The method of item 6795 wherein the agent is a TACE inhibitor.

6900. The method of item 6795 wherein the agent is a tyrosine kinase inhibitor.

6901. The method of item 6795 wherein the agent is a vitronectin inhibitor.

6902. The method of item 6795 wherein the agent is a fibroblast growth factor inhibitor.

6903. The method of item 6795 wherein the agent is a protein kinase inhibitor.

6904. The method of item 6795 wherein the agent is a PDGF receptor kinase inhibitor.

6905. The method of item 6795 wherein the agent is an endothelial growth factor receptor kinase inhibitor.

6906. The method of item 6795 wherein the agent is a retinoic acid receptor antagonist.

6907. The method of item 6795 wherein the agent is a platelet derived growth factor receptor kinase inhibitor.

6908. The method of item 6795 wherein the agent is a fibronogin antagonist.

6909. The method of item 6795 wherein the agent is an antimycotic agent.

6910. The method of item 6795 wherein the agent is an antimycotic agent, wherein the antimycotic agent is sulconizole.

6911. The method of item 6795 wherein the agent is a bisphosphonate.

6912. The method of item 6795 wherein the agent is a phospholipase A1 inhibitor.

6913. The method of item 6795 wherein the agent is a histamine H1/H2/H3 receptor antagonist.

6914. The method of item 6795 wherein the agent is a macrolide antibiotic.

6915. The method of item 6795 wherein the agent is a GPIIb/IIIa receptor antagonist.

6916. The method of item 6795 wherein the agent is an endothelin receptor antagonist.

6917. The method of item 6795 wherein the agent is a peroxisome proliferator-activated receptor agonist.

6918. The method of item 6795 wherein the agent is an estrogen receptor agent.

6919. The method of item 6795 wherein the agent is a somastostatin analogue.

6920. The method of item 6795 wherein the agent is a neurokinin 1 antagonist.

6921. The method of item 6795 wherein the agent is a neurokinin 3 antagonist.

6922. The method of item 6795 wherein the agent is a VLA-4 antagonist.

6923. The method of item 6795 wherein the agent is an osteoclast inhibitor.

6924. The method of item 6795 wherein the agent is a DNA topoisomerase ATP hydrolyzing inhibitor.

6925. The method of item 6795 wherein the agent is an angiotensin I converting enzyme inhibitor.

6926. The method of item 6795 wherein the agent is an angiotensin II antagonist.

6927. The method of item 6795 wherein the agent is an enkephalinase inhibitor.

6928. The method of item 6795 wherein the agent is a peroxisome proliferator-activated receptor gamma agonist insulin sensitizer.

6929. The method of item 6795 wherein the agent is a protein kinase C inhibitor.

6930. The method of item 6795 wherein the agent is a ROCK (rho-associated kinase) inhibitor.

6931. The method of item 6795 wherein the agent is a CXCR3 inhibitor.

6932. The method of item 6795 wherein the agent is an Itk inhibitor.

6933. The method of item 6795 wherein the agent is a cytosolic phospholipase A₂-alpha inhibitor.

6934. The method of item 6795 wherein the agent is a PPAR agonist.

6935. The method of item 6795 wherein the agent is an immunosuppressant.

6936. The method of item 6795 wherein the agent is an Erb inhibitor.

6937. The method of item 6795 wherein the agent is an apoptosis agonist.

6938. The method of item 6795 wherein the agent is a lipocortin agonist.

6939. The method of item 6795 wherein the agent is a VCAM-1 antagonist.

6940. The method of item 6795 wherein the agent is a collagen antagonist.

6941. The method of item 6795 wherein the agent is an alpha 2 integrin antagonist.

6942. The method of item 6795 wherein the agent is a TNF alpha inhibitor.

6943. The method of item 6795 wherein the agent is a nitric oxide inhibitor.

6944. The method of item 6795 wherein the agent is a cathepsin inhibitor.

6945. The method of item 6795 wherein the agent is not an anti-inflammatory agent.

6946. The method of item 6795 wherein the agent is not a steroid.

6947. The method of item 6795 wherein the agent is not a glucocorticosteroid.

6948. The method of item 6795 wherein the agent is not dexamethasone.

6949. The method of item 6795 wherein the agent is not an anti-infective agent.

6950. The method of item 6795 wherein the agent is not an antibiotic.

6951. The method of item 6795 wherein the agent is not an anti-fungal agent.

6952. The method of item 6795, wherein the composition comprises a polymer.

6953. The method of item 6795, wherein the composition comprises a polymer, and the polymer is, or comprises, a copolymer.

6954. The method of item 6795, wherein the composition comprises a polymer, and the polymer is, or comprises, a block copolymer.

6955. The method of item 6795, wherein the composition comprises a polymer, and the polymer is, or comprises, a random copolymer.

6956. The method of item 6795, wherein the composition comprises a polymer, and the polymer is, or comprises, a biodegradable polymer.

6957. The method of item 6795, wherein the composition comprises a polymer, and the polymer is, or comprises, a non-biodegradable polymer.

6958. The method of item 6795, wherein the composition comprises a polymer, and the polymer is, or comprises, a hydrophilic polymer.

6959. The method of item 6795, wherein the composition comprises a polymer, and the polymer is, or comprises, a hydrophobic polymer.

6960. The method of item 6795, wherein the composition comprises a polymer, and the polymer is, or comprises, a polymer having hydrophilic domains.

6961. The method of item 6795, wherein the composition comprises a polymer, and the polymer is, or comprises, a polymer having hydrophobic domains.

6962. The method of item 6795, wherein the composition comprises a polymer, and the polymer is, or comprises, a non-conductive polymer.

6963. The method of item 6795, wherein the composition comprises a polymer, and the polymer is, or comprises, an elastomer.

6964. The method of item 6795, wherein the composition comprises a polymer, and the polymer is, or comprises, a hydrogel.

6965. The method of item 6795, wherein the composition comprises a polymer, and the polymer is, or comprises, a silicone polymer.

6966. The method of item 6795, wherein the composition comprises a polymer, and the polymer is, or comprises, a hydrocarbon polymer.

6967. The method of item 6795, wherein the composition comprises a polymer, and the polymer is, or comprises, a styrene-derived polymer.

6968. The method of item 6795, wherein the composition comprises a polymer, and the polymer is, or comprises, a butadiene-derived polymer.

6969. The method of item 6795, wherein the composition comprises a polymer, and the polymer is, or comprises, a macromer.

6970. The method of item 6795, wherein the composition comprises a polymer, and the polymer is, or comprises, a poly(ethylene glycol)polymer.

6971. The method of item 6795, wherein the composition comprises a polymer, and the polymer is, or comprises, an amorphous polymer.

6972. The method of item 6795, wherein the composition further comprises a second pharmaceutically active agent.

6973. The method of item 6795, wherein the composition further comprises an anti-inflammatory agent.

6974. The method of item 6795, wherein the composition further comprises an agent that inhibits infection.

6975. The method of item 6795, wherein the composition further comprises an anthracycline.

6976. The method of item 6795, wherein the composition further comprises doxorubicin.

6977. The method of item 6795 wherein the composition further comprises mitoxantrone.

6978. The method of item 6795 wherein the composition further comprises a fluoropyrimidine.

6979. The method of item 6795, wherein the composition further comprises 5-fluorouracil (5-FU).

6980. The method of item 6795, wherein the composition further comprises a folic acid antagonist.

6981. The method of item 6795, wherein the composition further comprises methotrexate.

6982. The method of item 6795, wherein the composition further comprises a podophylotoxin.

6983. The method of item 6795, wherein the composition further comprises etoposide.

6984. The method of item 6795, wherein the composition further comprises camptothecin.

6985. The method of item 6795, wherein the composition further comprises a hydroxyurea.

6986. The method of item 6795, wherein the composition further comprises a platinum complex.

6987. The method of item 6795, wherein the composition further comprises cisplatin.

6988. The method of item 6795 wherein the composition further comprises an anti-thrombotic agent.

6989. The method of item 6795, wherein the composition further comprises a visualization agent.

6990. The method of item 6795, wherein the composition further comprises a visualization agent, and the visualization agent is a radiopaque material, wherein the radiopaque material comprises a metal, a halogenated compound, or a barium containing compound.

6991. The method of item 6795, wherein the composition further comprises a visualization agent, and the visualization agent is, or comprises, barium, tantalum, or technetium.

6992. The method of item 6795, wherein the composition further comprises a visualization agent, and the visualization agent is, or comprises, an MRI responsive material.

6993. The method of item 6795, wherein the composition further comprises a visualization agent, and the visualization agent is, or comprises, a gadolinium chelate.

6994. The method of item 6795, wherein the composition further comprises a visualization agent, and the visualization agent is, or comprises, iron, magnesium, manganese, copper, or chromium.

6995. The method of item 6795, wherein the composition further comprises a visualization agent, and the visualization agent is, or comprises, iron oxide compound.

6996. The method of item 6795, wherein the composition further comprises a visualization agent, and the visualization agent is, or comprises, a dye, pigment, or colorant.

6997. The method of item 6795 wherein the agent is released in effective concentrations from the composition comprising the agent by diffusion over a period ranging from the time of administration to about 90 days.

6998. The method of item 6795 wherein the agent is released in effective concentrations from the composition comprising the agent by erosion of the composition over a period ranging from the time of administration to about 90 days.

6999. The method of item 6795 wherein the composition further comprises an inflammatory cytokine.

7000. The method of item 6795 wherein the composition further comprises an agent that stimulates cell proliferation.

7001. The method of item 6795 wherein the composition further comprises a polymeric carrier.

7002. The method of item 6795 wherein the composition is in the form of a gel, paste, or spray.

7003. The method of item 6795 wherein the implant is partially constructed with the agent or the composition.

7004. The method of item 6795 wherein the implant is fully constructed with the agent or the composition.

7005. The method of item 6795 wherein the implant is impregnated with the agent or the composition.

7006. The method of item 6795, wherein the agent or the composition forms a coating, and the coating directly contacts the implant.

7007. The method of item 6795, wherein the agent or the composition forms a coating, and the coating indirectly contacts the implant.

7008. The method of item 6795 wherein the agent or the composition forms a coating, and the coating partially covers the implant.

7009. The method of item 6795, wherein the agent or the composition forms a coating, and the coating completely covers the implant.

7010. The method of item 6795 wherein the agent or the composition is located within pores or holes of the implant.

7011. The method of item 6795 wherein the agent or the composition is located within a channel, lumen, or divet of the implant.

7012. The method of item 6795 wherein the implant further comprising an echogenic material.

7013. The method of item 6795 wherein the implant further comprises an echogenic material, wherein the echogenic material is in the form of a coating.

7014. The method of item 6795 wherein the implant is sterile.

7015. The method of item 6795 wherein the agent is delivered from the implant, wherein the agent is released into tissue in the vicinity of the implant after deployment of the implant.

7016. The method of item 6795 wherein the agent is delivered from the implant, wherein the agent is released into tissue in the vicinity of the implant after deployment of the implant, wherein the tissue is connective tissue.

7017. The method of item 6795 wherein the agent is delivered from the implant, wherein the agent is released into tissue in the vicinity of the implant after deployment of the implant, wherein the tissue is muscle tissue.

7018. The method of item 6795 wherein the agent is delivered from the implant, wherein the agent is released into tissue in the vicinity of the implant after deployment of the implant, wherein the tissue is nerve tissue.

7019. The method of item 6795 wherein the agent is delivered from the implant, wherein the agent is released into tissue in the vicinity of the implant after deployment of the implant, wherein the tissue is epithelium tissue.

7020. The method of item 6795 wherein the agent is delivered from the implant, wherein the agent is released in effective concentrations from the implant over a period ranging from the time of deployment of the implant to about 1 year.

7021. The method of item 6795 wherein the agent is delivered from the implant, wherein the agent is released in effective concentrations from the implant over a period ranging from about 1 month to 6 months.

7022. The method of item 6795 wherein the agent is delivered from the implant, wherein the agent is released in effective concentrations from the implant over a period ranging from about 1-90 days.

7023. The method of item 6795 wherein the agent is delivered from the implant, wherein the agent is released in effective concentrations from the implant at a constant rate.

7024. The method of item 6795 wherein the agent is delivered from the implant, wherein the agent is released in effective concentrations from the implant at an increasing rate.

7025. The method of item 6795 wherein the agent is delivered from the implant, wherein the agent is released in effective concentrations from the implant at a decreasing rate.

7026. The method of item 6795 wherein the agent is delivered from the implant, wherein the implant comprises about 0.01 μg to about 10 μg of the agent.

7027. The method of item 6795 wherein the agent is delivered from the implant, wherein the implant comprises about 10 μg to about 10 mg of the agent.

7028. The method of item 6795 wherein the agent is delivered from the implant, wherein the implant comprises about 10 mg to about 250 mg of the agent.

7029. The method of item 6795 wherein the agent is delivered from the implant, wherein the implant comprises about 250 mg to about 1000 mg of the agent.

7030. The method of item 6795 wherein the agent is delivered from the implant, wherein the implant comprises about 1000 mg to about 2500 mg of the agent.

7031. The method of item 6795 wherein the agent is delivered from the implant, wherein a surface of the implant comprises less than 0.01 μg of the agent per mm² of implant surface to which the agent is applied.

7032. The method of item 6795 wherein the agent is delivered from the implant, wherein a surface of the implant comprises about 0.01 μg to about 1 μg of the agent per mm² of implant surface to which the agent is applied.

7033. The method of item 6795 wherein the agent is delivered from the implant, wherein a surface of the implant comprises about 1 μg to about 10 μg of the agent per mm² of implant surface to which the agent is applied.

7034. The method of item 6795 wherein the agent is delivered from the implant, wherein a surface of the implant comprises about 10 μg to about 250 μg of the agent per mm² of implant surface to which the agent is applied.

7035. The method of item 6795 wherein the agent is delivered from the implant, wherein a surface of the implant comprises about 250 μg to about 1000 μg of the agent per mm² of implant surface to which the agent is applied.

7036. The method of item 6795 wherein the agent is delivered from the implant, wherein a surface of the implant comprises about 1000 μg to about 2500 μg of the agent per mm² of implant surface to which the agent is applied.

7037. The method of item 6795, wherein the implant further comprises a coating, and the coating is a uniform coating.

7038. The method of item 6795, wherein the implant further comprises a coating, and the coating is a non-uniform coating.

7039. The method of item 6795, wherein the implant further comprises a coating, and the coating is a discontinuous coating.

7040. The method of item 6795, wherein the implant further comprises a coating, and the coating is a patterned coating.

7041. The method of item 6795, wherein the implant further comprises a coating, and the coating has a thickness of 100 μm or less.

7042. The method of item 6795, wherein the implant further comprises a coating, and the coating has a thickness of 10 μm or less.

7043. The method of item 6795, wherein the implant further comprises a coating, and the coating adheres to the surface of the implant upon deployment of the implant.

7044. The method of item 6795, wherein the implant further comprises a coating, and the coating is stable at room temperature for a period of at least 1 year.

7045. The method of item 6795, wherein the implant further comprises a coating, and the agent is present in the coating in an amount ranging between about 0.0001% to about 1% by weight.

7046. The method of item 6795, wherein the implant further comprises a coating, and the agent is present in the coating in an amount ranging between about 1% to about 10% by weight.

7047. The method of item 6795, wherein the implant further comprises a coating, and the agent is present in the coating in an amount ranging between about 10% to about 25% by weight.

7048. The method of item 6795, wherein the implant further comprises a coating, and the agent is present in the coating in an amount ranging between about 25% to about 70% by weight.

7049. The method of item 6795, wherein the implant further comprises a coating, and the coating comprises a polymer.

7050. The method of item 6795, wherein the implant comprises a first coating having a first composition and a second coating having a second composition.

7051. The method of item 6795, wherein the implant comprises a first coating having a first composition and a second coating having a second composition, wherein the first composition and the second composition are different.

7052. A method for inhibiting scarring comprising placing a peritoneal dialysis catheter (i.e., an implant) and an anti-scarring agent or a composition comprising an anti-scarring agent into an animal host, wherein the agent inhibits scarring.

7053. The method of item 7052 wherein the agent inhibits cell regeneration.

7054. The method of item 7052 wherein the agent inhibits angiogenesis.

7055. The method of item 7052 wherein the agent inhibits fibroblast migration.

7056. The method of item 7052 wherein the agent inhibits fibroblast proliferation.

7057. The method of item 7052 wherein the agent inhibits deposition of extracellular matrix.

7058. The method of item 7052 wherein the agent inhibits tissue remodeling.

7059. The method of item 7052 wherein the agent is an angiogenesis inhibitor.

7060. The method of item 7052 wherein the agent is a 5-lipoxygenase inhibitor or antagonist.

7061. The method of item 7052 wherein the agent is a chemokine receptor antagonist.

7062. The method of item 7052 wherein the agent is a cell cycle inhibitor.

7063. The method of item 7052 wherein the agent is a taxane.

7064. The method of item 7052 wherein the agent is an anti-microtubule agent.

7065. The method of item 7052 wherein the agent is paclitaxel.

7066. The method of item 7052 wherein the agent is not paclitaxel.

7067. The method of item 7052 wherein the agent is an analogue or derivative of paclitaxel.

7068. The method of item 7052 wherein the agent is a vinca alkaloid.

7069. The method of item 7052 wherein the agent is camptothecin or an analogue or derivative thereof.

7070. The method of item 7052 wherein the agent is a podophyllotoxin.

7071. The method of item 7052 wherein the agent is a podophyllotoxin, wherein the podophyllotoxin is etoposide or an analogue or derivative thereof.

7072. The method of item 7052 wherein the agent is an anthracycline.

7073. The method of item 7052 wherein the agent is an anthracycline, wherein the anthracycline is doxorubicin or an analogue or derivative thereof.

7074. The method of item 7052 wherein the agent is an anthracycline, wherein the anthracycline is mitoxantrone or an analogue or derivative thereof.

7075. The method of item 7052 wherein the agent is a platinum compound.

7076. The method of item 7052 wherein the agent is a nitrosourea.

7077. The method of item 7052 wherein the agent is a nitroimidazole.

7078. The method of item 7052 wherein the agent is a folic acid antagonist.

7079. The method of item 7052 wherein the agent is a cytidine analogue.

7080. The method of item 7052 wherein the agent is a pyrimidine analogue.

7081. The method of item 7052 wherein the agent is a fluoropyrimidine analogue.

7082. The method of item 7052 wherein the agent is a purine analogue.

7083. The method of item 7052 wherein the agent is a nitrogen mustard or an analogue or derivative thereof.

7084. The method of item 7052 wherein the agent is a hydroxyurea.

7085. The method of item 7052 wherein the agent is a mytomicin or an analogue or derivative thereof.

7086. The method of item 7052 wherein the agent is an alkyl sulfonate.

7087. The method of item 7052 wherein the agent is a benzamide or an analogue or derivative thereof.

7088. The method of item 7052 wherein the agent is a nicotinamide or an analogue or derivative thereof.

7089. The method of item 7052 wherein the agent is a halogenated sugar or an analogue or derivative thereof.

7090. The method of item 7052 wherein the agent is a DNA alkylating agent.

7091. The method of item 7052 wherein the agent is an anti-microtubule agent.

7092. The method of item 7052 wherein the agent is a topoisomerase inhibitor.

7093. The method of item 7052 wherein the agent is a DNA cleaving agent.

7094. The method of item 7052 wherein the agent is an antimetabolite.

7095. The method of item 7052 wherein the agent inhibits adenosine deaminase.

7096. The method of item 7052 wherein the agent inhibits purine ring synthesis.

7097. The method of item 7052 wherein the agent is a nucleotide interconversion inhibitor.

7098. The method of item 7052 wherein the agent inhibits dihydrofolate reduction.

7099. The method of item 7052 wherein the agent blocks thymidine monophosphate.

7100. The method of item 7052 wherein the agent causes DNA damage.

7101. The method of item 7052 wherein the agent is a DNA intercalation agent.

7102. The method of item 7052 wherein the agent is a RNA synthesis inhibitor.

7103. The method of item 7052 wherein the agent is a pyrimidine synthesis inhibitor.

7104. The method of item 7052 wherein the agent inhibits ribonucleotide synthesis or function.

7105. The method of item 7052 wherein the agent inhibits thymidine monophosphate synthesis or function.

7106. The method of item 7052 wherein the agent inhibits DNA synthesis.

7107. The method of item 7052 wherein the agent causes DNA adduct formation.

7108. The method of item 7052 wherein the agent inhibits protein synthesis.

7109. The method of item 7052 wherein the agent inhibits microtubule function.

7110. The method of item 7052 wherein the agent is a cyclin dependent protein kinase inhibitor.

7111. The method of item 7052 wherein the agent is an epidermal growth factor kinase inhibitor.

7112. The method of item 7052 wherein the agent is an elastase inhibitor.

7113. The method of item 7052 wherein the agent is a factor Xa inhibitor.

7114. The method of item 7052 wherein the agent is a farnesyltransferase inhibitor.

7115. The method of item 7052 wherein the agent is a fibrinogen antagonist.

7116. The method of item 7052 wherein the agent is a guanylate cyclase stimulant.

7117. The method of item 7052 wherein the agent is a heat shock protein 90 antagonist.

7118. The method of item 7052 wherein the agent is a heat shock protein 90 antagonist, wherein the heat shock protein 90 antagonist is geldanamycin or an analogue or derivative thereof.

7119. The method of item 7052 wherein the agent is a guanylate cyclase stimulant.

7120. The method of item 7052 wherein the agent is a HMGCoA reductase inhibitor.

7121. The method of item 7052 wherein the agent is a HMGCoA reductase inhibitor, wherein the HMGCoA reductase inhibitor is simvastatin or an analogue or derivative thereof.

7122. The method of item 7052 wherein the agent is a hydroorotate dehydrogenase inhibitor.

7123. The method of item 7052 wherein the agent is an IKK2 inhibitor.

7124. The method of item 7052 wherein the agent is an IL-1 antagonist.

7125. The method of item 7052 wherein the agent is an ICE antagonist.

7126. The method of item 7052 wherein the agent is an IRAK antagonist.

7127. The method of item 7052 wherein the agent is an IL-4 agonist.

7128. The method of item 7052 wherein the agent is an immunomodulatory agent.

7129. The method of item 7052 wherein the agent is sirolimus or an analogue or derivative thereof.

7130. The method of item 7052 wherein the agent is not sirolimus.

7131. The method of item 7052 wherein the agent is everolimus or an analogue or derivative thereof.

7132. The method of item 7052 wherein the agent is tacrolimus or an analogue or derivative thereof.

7133. The method of item 7052 wherein the agent is not tacrolimus.

7134. The method of item 7052 wherein the agent is biolmus or an analogue or derivative thereof.

7135. The method of item 7052 wherein the agent is tresperimus or an analogue or derivative thereof.

7136. The method of item 7052 wherein the agent is auranofin or an analogue or derivative thereof.

7137. The method of item 7052 wherein the agent is 27-0-demethylrapamycin or an analogue or derivative thereof.

7138. The method of item 7052 wherein the agent is gusperimus or an analogue or derivative thereof.

7139. The method of item 7052 wherein the agent is pimecrolimus or an analogue or derivative thereof.

7140. The method of item 7052 wherein the agent is ABT-578 or an analogue or derivative thereof.

7141. The method of item 7052 wherein the agent is an inosine monophosphate dehydrogenase (IMPDH) inhibitor.

7142. The method of item 7052 wherein the agent is an IMPDH inhibitor, wherein the IMPDH inhibitor is mycophenolic acid or an analogue or derivative thereof.

7143. The method of item 7052 wherein the agent is an IMPDH inhibitor, wherein the IMPDH inhibitor is 1-alpha-25 dihydroxy vitamin D₃ or an analogue or derivative thereof.

7144. The method of item 7052 wherein the agent is a leukotriene inhibitor.

7145. The method of item 7052 wherein the agent is a MCP-1 antagonist.

7146. The method of item 7052 wherein the agent is a MMP inhibitor.

7147. The method of item 7052 wherein the agent is an NF kappa B inhibitor.

7148. The method of item 7052 wherein the agent is an NF kappa B inhibitor, wherein the NF kappa B inhibitor is Bay 11-7082.

7149. The method of item 7052 wherein the agent is an NO agonist.

7150. The method of item 7052 wherein the agent is a p38 MAP kinase inhibitor.

7151. The method of item 7052 wherein the agent is a p38 MAP kinase inhibitor, wherein the p38 MAP kinase inhibitor is SB 202190.

7152. The method of item 7052 wherein the agent is a phosphodiesterase inhibitor.

7153. The method of item 7052 wherein the agent is a TGF beta inhibitor.

7154. The method of item 7052 wherein the agent is a thromboxane A2 antagonist.

7155. The method of item 7052 wherein the agent is a TNFa antagonist.

7156. The method of item 7052 wherein the agent is a TACE inhibitor.

7157. The method of item 7052 wherein the agent is a tyrosine kinase inhibitor.

7158. The method of item 7052 wherein the agent is a vitronectin inhibitor.

7159. The method of item 7052 wherein the agent is a fibroblast growth factor inhibitor.

7160. The method of item 7052 wherein the agent is a protein kinase inhibitor.

7161. The method of item 7052 wherein the agent is a PDGF receptor kinase inhibitor.

7162. The method of item 7052 wherein the agent is an endothelial growth factor receptor kinase inhibitor.

7163. The method of item 7052 wherein the agent is a retinoic acid receptor antagonist.

7164. The method of item 7052 wherein the agent is a platelet derived growth factor receptor kinase inhibitor.

7165. The method of item 7052 wherein the agent is a fibronogin antagonist.

7166. The method of item 7052 wherein the agent is an antimycotic agent.

7167. The method of item 7052 wherein the agent is an antimycotic agent, wherein the antimycotic agent is sulconizole.

7168. The method of item 7052 wherein the agent is a bisphosphonate.

7169. The method of item 7052 wherein the agent is a phospholipase A1 inhibitor.

7170. The method of item 7052 wherein the agent is a histamine H1/H2/H3 receptor antagonist.

7171. The method of item 7052 wherein the agent is a macrolide antibiotic.

7172. The method of item 7052 wherein the agent is a GPIIb/IIIa receptor antagonist.

7173. The method of item 7052 wherein the agent is an endothelin receptor antagonist.

7174. The method of item 7052 wherein the agent is a peroxisome proliferator-activated receptor agonist.

7175. The method of item 7052 wherein the agent is an estrogen receptor agent.

7176. The method of item 7052 wherein the agent is a somastostatin analogue.

7177. The method of item 7052 wherein the agent is a neurokinin 1 antagonist.

7178. The method of item 7052 wherein the agent is a neurokinin 3 antagonist.

7179. The method of item 7052 wherein the agent is a VLA-4 antagonist.

7180. The method of item 7052 wherein the agent is an osteoclast inhibitor.

7181. The method of item 7052 wherein the agent is a DNA topoisomerase ATP hydrolyzing inhibitor.

7182. The method of item 7052 wherein the agent is an angiotensin I converting enzyme inhibitor.

7183. The method of item 7052 wherein the agent is an angiotensin II antagonist.

7184. The method of item 7052 wherein the agent is an enkephalinase inhibitor.

7185. The method of item 7052 wherein the agent is a peroxisome proliferator-activated receptor gamma agonist insulin sensitizer.

7186. The method of item 7052 wherein the agent is a protein kinase C inhibitor.

7187. The method of item 7052 wherein the agent is a ROCK (rho-associated kinase) inhibitor.

7188. The method of item 7052 wherein the agent is a CXCR3 inhibitor.

7189. The method of item 7052 wherein the agent is an Itk inhibitor.

7190. The method of item 7052 wherein the agent is a cytosolic phospholipase A₂-alpha inhibitor.

7191. The method of item 7052 wherein the agent is a PPAR agonist.

7192. The method of item 7052 wherein the agent is an immunosuppressant.

7193. The method of item 7052 wherein the agent is an Erb inhibitor.

7194. The method of item 7052 wherein the agent is an apoptosis agonist.

7195. The method of item 7052 wherein the agent is a lipocortin agonist.

7196. The method of item 7052 wherein the agent is a VCAM-1 antagonist.

7197. The method of item 7052 wherein the agent is a collagen antagonist.

7198. The method of item 7052 wherein the agent is an alpha 2 integrin antagonist.

7199. The method of item 7052 wherein the agent is a TNF alpha inhibitor.

7200. The method of item 7052 wherein the agent is a nitric oxide inhibitor.

7201. The method of item 7052 wherein the agent is a cathepsin inhibitor.

7202. The method of item 7052 wherein the agent is not an anti-inflammatory agent.

7203. The method of item 7052 wherein the agent is not a steroid.

7204. The method of item 7052 wherein the agent is not a glucocorticosteroid.

7205. The method of item 7052 wherein the agent is not dexamethasone.

7206. The method of item 7052 wherein the agent is not an anti-infective agent.

7207. The method of item 7052 wherein the agent is not an antibiotic.

7208. The method of item 7052 wherein the agent is not an anti-fungal agent.

7209. The method of item 7052, wherein the composition comprises a polymer.

7210. The method of item 7052, wherein the composition comprises a polymer, and the polymer is, or comprises, a copolymer.

7211. The method of item 7052, wherein the composition comprises a polymer, and the polymer is, or comprises, a block copolymer.

7212. The method of item 7052, wherein the composition comprises a polymer, and the polymer is, or comprises, a random copolymer.

7213. The method of item 7052, wherein the composition comprises a polymer, and the polymer is, or comprises, a biodegradable polymer.

7214. The method of item 7052, wherein the composition comprises a polymer, and the polymer is, or comprises, a non-biodegradable polymer.

7215. The method of item 7052, wherein the composition comprises a polymer, and the polymer is, or comprises, a hydrophilic polymer.

7216. The method of item 7052, wherein the composition comprises a polymer, and the polymer is, or comprises, a hydrophobic polymer.

7217. The method of item 7052, wherein the composition comprises a polymer, and the polymer is, or comprises, a polymer having hydrophilic domains.

7218. The method of item 7052, wherein the composition comprises a polymer, and the polymer is, or comprises, a polymer having hydrophobic domains.

7219. The method of item 7052, wherein the composition comprises a polymer, and the polymer is, or comprises, a non-conductive polymer.

7220. The method of item 7052, wherein the composition comprises a polymer, and the polymer is, or comprises, an elastomer.

7221. The method of item 7052, wherein the composition comprises a polymer, and the polymer is, or comprises, a hydrogel.

7222. The method of item 7052, wherein the composition comprises a polymer, and the polymer is, or comprises, a silicone polymer.

7223. The method of item 7052, wherein the composition comprises a polymer, and the polymer is, or comprises, a hydrocarbon polymer.

7224. The method of item 7052, wherein the composition comprises a polymer, and the polymer is, or comprises, a styrene-derived polymer.

7225. The method of item 7052, wherein the composition comprises a polymer, and the polymer is, or comprises, a butadiene-derived polymer.

7226. The method of item 7052, wherein the composition comprises a polymer, and the polymer is, or comprises, a macromer.

7227. The method of item 7052, wherein the composition comprises a polymer, and the polymer is, or comprises, a poly(ethylene glycol)polymer.

7228. The method of item 7052, wherein the composition comprises a polymer, and the polymer is, or comprises, an amorphous polymer.

7229. The method of item 7052, wherein the composition further comprises a second pharmaceutically active agent.

7230. The method of item 7052, wherein the composition further comprises an anti-inflammatory agent.

7231. The method of item 7052, wherein the composition further comprises an agent that inhibits infection.

7232. The method of item 7052, wherein the composition further comprises an anthracycline.

7233. The method of item 7052, wherein the composition further comprises doxorubicin.

7234. The method of item 7052 wherein the composition further comprises mitoxantrone.

7235. The method of item 7052 wherein the composition further comprises a fluoropyrimidine.

7236. The method of item 7052, wherein the composition further comprises 5-fluorouracil (5-FU).

7237. The method of item 7052, wherein the composition further comprises a folic acid antagonist.

7238. The method of item 7052, wherein the composition further comprises methotrexate.

7239. The method of item 7052, wherein the composition further comprises a podophylotoxin.

7240. The method of item 7052, wherein the composition further comprises etoposide.

7241. The method of item 7052, wherein the composition further comprises camptothecin.

7242. The method of item 7052, wherein the composition further comprises a hydroxyurea.

7243. The method of item 7052, wherein the composition further comprises a platinum complex.

7244. The method of item 7052, wherein the composition further comprises cisplatin.

7245. The method of item 7052 wherein the composition further comprises an anti-thrombotic agent.

7246. The method of item 7052, wherein the composition further comprises a visualization agent.

7247. The method of item 7052, wherein the composition further comprises a visualization agent, and the visualization agent is a radiopaque material, wherein the radiopaque material comprises a metal, a halogenated compound, or a barium containing compound.

7248. The method of item 7052, wherein the composition further comprises a visualization agent, and the visualization agent is, or comprises, barium, tantalum, or technetium.

7249. The method of item 7052, wherein the composition further comprises a visualization agent, and the visualization agent is, or comprises, an MRI responsive material.

7250. The method of item 7052, wherein the composition further comprises a visualization agent, and the visualization agent is, or comprises, a gadolinium chelate.

7251. The method of item 7052, wherein the composition further comprises a visualization agent, and the visualization agent is, or comprises, iron, magnesium, manganese, copper, or chromium.

7252. The method of item 7052, wherein the composition further comprises a visualization agent, and the visualization agent is, or comprises, iron oxide compound.

7253. The method of item 7052, wherein the composition further comprises a visualization agent, and the visualization agent is, or comprises, a dye, pigment, or colorant.

7254. The method of item 7052 wherein the agent is released in effective concentrations from the composition comprising the agent by diffusion over a period ranging from the time of administration to about 90 days.

7255. The method of item 7052 wherein the agent is released in effective concentrations from the composition comprising the agent by erosion of the composition over a period ranging from the time of administration to about 90 days.

7256. The method of item 7052 wherein the composition further comprises an inflammatory cytokine.

7257. The method of item 7052 wherein the composition further comprises an agent that stimulates cell proliferation.

7258. The method of item 7052 wherein the composition further comprises a polymeric carrier.

7259. The method of item 7052 wherein the composition is in the form of a gel, paste, or spray.

7260. The method of item 7052 wherein the implant is partially constructed with the agent or the composition.

7261. The method of item 7052 wherein the implant is fully constructed with the agent or the composition.

7262. The method of item 7052 wherein the implant is impregnated with the agent or the composition.

7263. The method of item 7052, wherein the agent or the composition forms a coating, and the coating directly contacts the implant.

7264. The method of item 7052, wherein the agent or the composition forms a coating, and the coating indirectly contacts the implant.

7265. The method of item 7052 wherein the agent or the composition forms a coating, and the coating partially covers the implant.

7266. The method of item 7052, wherein the agent or the composition forms a coating, and the coating completely covers the implant.

7267. The method of item 7052 wherein the agent or the composition is located within pores or holes of the implant.

7268. The method of item 7052 wherein the agent or the composition is located within a channel, lumen, or divet of the implant.

7269. The method of item 7052 wherein the implant further comprising an echogenic material.

7270. The method of item 7052 wherein the implant further comprises an echogenic material, wherein the echogenic material is in the form of a coating.

7271. The method of item 7052 wherein the implant is sterile.

7272. The method of item 7052 wherein the agent is delivered from the implant, wherein the agent is released into tissue in the vicinity of the implant after deployment of the implant.

7273. The method of item 7052 wherein the agent is delivered from the implant, wherein the agent is released into tissue in the vicinity of the implant after deployment of the implant, wherein the tissue is connective tissue.

7274. The method of item 7052 wherein the agent is delivered from the implant, wherein the agent is released into tissue in the vicinity of the implant after deployment of the implant, wherein the tissue is muscle tissue.

7275. The method of item 7052 wherein the agent is delivered from the implant, wherein the agent is released into tissue in the vicinity of the implant after deployment of the implant, wherein the tissue is nerve tissue.

7276. The method of item 7052 wherein the agent is delivered from the implant, wherein the agent is released into tissue in the vicinity of the implant after deployment of the implant, wherein the tissue is epithelium tissue.

7277. The method of item 7052 wherein the agent is delivered from the implant, wherein the agent is released in effective concentrations from the implant over a period ranging from the time of deployment of the implant to about 1 year.

7278. The method of item 7052 wherein the agent is delivered from the implant, wherein the agent is released in effective concentrations from the implant over a period ranging from about 1 month to 6 months.

7279. The method of item 7052 wherein the agent is delivered from the implant, wherein the agent is released in effective concentrations from the implant over a period ranging from about 1-90 days.

7280. The method of item 7052 wherein the agent is delivered from the implant, wherein the agent is released in effective concentrations from the implant at a constant rate.

7281. The method of item 7052 wherein the agent is delivered from the implant, wherein the agent is released in effective concentrations from the implant at an increasing rate.

7282. The method of item 7052 wherein the agent is delivered from the implant, wherein the agent is released in effective concentrations from the implant at a decreasing rate.

7283. The method of item 7052 wherein the agent is delivered from the implant, wherein the implant comprises about 0.01 μg to about 10 μg of the agent.

7284. The method of item 7052 wherein the agent is delivered from the implant, wherein the implant comprises about 10 μg to about 10 mg of the agent.

7285. The method of item 7052 wherein the agent is delivered from the implant, wherein the implant comprises about 10 mg to about 250 mg of the agent.

7286. The method of item 7052 wherein the agent is delivered from the implant, wherein the implant comprises about 250 mg to about 1000 mg of the agent.

7287. The method of item 7052 wherein the agent is delivered from the implant, wherein the implant comprises about 1000 mg to about 2500 mg of the agent.

7288. The method of item 7052 wherein the agent is delivered from the implant, wherein a surface of the implant comprises less than 0.01 μg of the agent per mm² of implant surface to which the agent is applied.

7289. The method of item 7052 wherein the agent is delivered from the implant, wherein a surface of the implant comprises about 0.01 μg to about 1 μg of the agent per mm² of implant surface to which the agent is applied.

7290. The method of item 7052 wherein the agent is delivered from the implant, wherein a surface of the implant comprises about 1 μg to about 10 μg of the agent per mm2 of implant surface to which the agent is applied.

7291. The method of item 7052 wherein the agent is delivered from the implant, wherein a surface of the implant comprises about 10 μg to about 250 μg of the agent per mm² of implant surface to which the agent is applied.

7292. The method of item 7052 wherein the agent is delivered from the implant, wherein a surface of the implant comprises about 250 μg to about 1000 μg of the agent per mm² of implant surface to which the agent is applied.

7293. The method of item 7052 wherein the agent is delivered from the implant, wherein a surface of the implant comprises about 1000 μg to about 2500 μg of the agent per mm² of implant surface to which the agent is applied.

7294. The method of item 7052, wherein the implant further comprises a coating, and the coating is a uniform coating.

7295. The method of item 7052, wherein the implant further comprises a coating, and the coating is a non-uniform coating.

7296. The method of item 7052, wherein the implant further comprises a coating, and the coating is a discontinuous coating.

7297. The method of item 7052, wherein the implant further comprises a coating, and the coating is a patterned coating.

7298. The method of item 7052, wherein the implant further comprises a coating, and the coating has a thickness of 100 μm or less.

7299. The method of item 7052, wherein the implant further comprises a coating, and the coating has a thickness of 10 μm or less.

7300. The method of item 7052, wherein the implant further comprises a coating, and the coating adheres to the surface of the implant upon deployment of the implant.

7301. The method of item 7052, wherein the implant further comprises a coating, and the coating is stable at room temperature for a period of at least 1 year.

7302. The method of item 7052, wherein the implant further comprises a coating, and the agent is present in the coating in an amount ranging between about 0.0001% to about 1% by weight.

7303. The method of item 7052, wherein the implant further comprises a coating, and the agent is present in the coating in an amount ranging between about 1% to about 10% by weight.

7304. The method of item 7052, wherein the implant further comprises a coating, and the agent is present in the coating in an amount ranging between about 10% to about 25% by weight.

7305. The method of item 7052, wherein the implant further comprises a coating, and the agent is present in the coating in an amount ranging between about 25% to about 70% by weight.

7306. The method of item 7052, wherein the implant further comprises a coating, and the coating comprises a polymer.

7307. The method of item 7052, wherein the implant comprises a first coating having a first composition and a second coating having a second composition.

7308. The method of item 7052, wherein the implant comprises a first coating having a first composition and a second coating having a second composition, wherein the first composition and the second composition are different.

7309. A method for inhibiting scarring comprising placing an implantable nonvascular stent or tube (i.e., an implant) and an anti-scarring agent or a composition comprising an anti-scarring agent into an animal host, wherein the agent inhibits scarring.

7310. The method of item 7309 wherein the agent inhibits cell regeneration.

7311. The method of item 7309 wherein the agent inhibits angiogenesis.

7312. The method of item 7309 wherein the agent inhibits fibroblast migration.

7313. The method of item 7309 wherein the agent inhibits fibroblast proliferation.

7314. The method of item 7309 wherein the agent inhibits deposition of extracellular matrix.

7315. The method of item 7309 wherein the agent inhibits tissue remodeling.

7316. The method of item 7309 wherein the agent is an angiogenesis inhibitor.

7317. The method of item 7309 wherein the agent is a 5-lipoxygenase inhibitor or antagonist.

7318. The method of item 7309 wherein the agent is a chemokine receptor antagonist.

7319. The method of item 7309 wherein the agent is a cell cycle inhibitor.

7320. The method of item 7309 wherein the agent is a taxane.

7321. The method of item 7309 wherein the agent is an anti-microtubule agent.

7322. The method of item 7309 wherein the agent is paclitaxel.

7323. The method of item 7309 wherein the agent is not paclitaxel.

7324. The method of item 7309 wherein the agent is an analogue or derivative of paclitaxel.

7325. The method of item 7309 wherein the agent is a vinca alkaloid.

7326. The method of item 7309 wherein the agent is camptothecin or an analogue or derivative thereof.

7327. The method of item 7309 wherein the agent is a podophyllotoxin.

7328. The method of item 7309 wherein the agent is a podophyllotoxin, wherein the podophyllotoxin is etoposide or an analogue or derivative thereof.

7329. The method of item 7309 wherein the agent is an anthracycline.

7330. The method of item 7309 wherein the agent is an anthracycline, wherein the anthracycline is doxorubicin or an analogue or derivative thereof.

7331. The method of item 7309 wherein the agent is an anthracycline, wherein the anthracycline is mitoxantrone or an analogue or derivative thereof.

7332. The method of item 7309 wherein the agent is a platinum compound.

7333. The method of item 7309 wherein the agent is a nitrosourea.

7334. The method of item 7309 wherein the agent is a nitroimidazole.

7335. The method of item 7309 wherein the agent is a folic acid antagonist.

7336. The method of item 7309 wherein the agent is a cytidine analogue.

7337. The method of item 7309 wherein the agent is a pyrimidine analogue.

7338. The method of item 7309 wherein the agent is a fluoropyrimidine analogue.

7339. The method of item 7309 wherein the agent is a purine analogue.

7340. The method of item 7309 wherein the agent is a nitrogen mustard or an analogue or derivative thereof.

7341. The method of item 7309 wherein the agent is a hydroxyurea.

7342. The method of item 7309 wherein the agent is a mytomicin or an analogue or derivative thereof.

7343. The method of item 7309 wherein the agent is an alkyl sulfonate.

7344. The method of item 7309 wherein the agent is a benzamide or an analogue or derivative thereof.

7345. The method of item 7309 wherein the agent is a nicotinamide or an analogue or derivative thereof.

7346. The method of item 7309 wherein the agent is a halogenated sugar or an analogue or derivative thereof.

7347. The method of item 7309 wherein the agent is a DNA alkylating agent.

7348. The method of item 7309 wherein the agent is an anti-microtubule agent.

7349. The method of item 7309 wherein the agent is a topoisomerase inhibitor.

7350. The method of item 7309 wherein the agent is a DNA cleaving agent.

7351. The method of item 7309 wherein the agent is an antimetabolite.

7352. The method of item 7309 wherein the agent inhibits adenosine deaminase.

7353. The method of item 7309 wherein the agent inhibits purine ring synthesis.

7354. The method of item 7309 wherein the agent is a nucleotide interconversion inhibitor.

7355. The method of item 7309 wherein the agent inhibits dihydrofolate reduction.

7356. The method of item 7309 wherein the agent blocks thymidine monophosphate.

7357. The method of item 7309 wherein the agent causes DNA damage.

7358. The method of item 7309 wherein the agent is a DNA intercalation agent.

7359. The method of item 7309 wherein the agent is a RNA synthesis inhibitor.

7360. The method of item 7309 wherein the agent is a pyrimidine synthesis inhibitor.

7361. The method of item 7309 wherein the agent inhibits ribonucleotide synthesis or function.

7362. The method of item 7309 wherein the agent inhibits thymidine monophosphate synthesis or function.

7363. The method of item 7309 wherein the agent inhibits DNA synthesis.

7364. The method of item 7309 wherein the agent causes DNA adduct formation.

7365. The method of item 7309 wherein the agent inhibits protein synthesis.

7366. The method of item 7309 wherein the agent inhibits microtubule function.

7367. The method of item 7309 wherein the agent is a cyclin dependent protein kinase inhibitor.

7368. The method of item 7309 wherein the agent is an epidermal growth factor kinase inhibitor.

7369. The method of item 7309 wherein the agent is an elastase inhibitor.

7370. The method of item 7309 wherein the agent is a factor Xa inhibitor.

7371. The method of item 7309 wherein the agent is a farnesyltransferase inhibitor.

7372. The method of item 7309 wherein the agent is a fibrinogen antagonist.

7373. The method of item 7309 wherein the agent is a guanylate cyclase stimulant.

7374. The method of item 7309 wherein the agent is a heat shock protein 90 antagonist.

7375. The method of item 7309 wherein the agent is a heat shock protein 90 antagonist, wherein the heat shock protein 90 antagonist is geldanamycin or an analogue or derivative thereof.

7376. The method of item 7309 wherein the agent is a guanylate cyclase stimulant.

7377. The method of item 7309 wherein the agent is a HMGCoA reductase inhibitor.

7378. The method of item 7309 wherein the agent is a HMGCoA reductase inhibitor, wherein the HMGCoA reductase inhibitor is simvastatin or an analogue or derivative thereof.

7379. The method of item 7309 wherein the agent is a hydroorotate dehydrogenase inhibitor.

7380. The method of item 7309 wherein the agent is an IKK2 inhibitor.

7381. The method of item 7309 wherein the agent is an IL-1 antagonist.

7382. The method of item 7309 wherein the agent is an ICE antagonist.

7383. The method of item 7309 wherein the agent is an IRAK antagonist.

7384. The method of item 7309 wherein the agent is an IL-4 agonist.

7385. The method of item 7309 wherein the agent is an immunomodulatory agent.

7386. The method of item 7309 wherein the agent is sirolimus or an analogue or derivative thereof.

7387. The method of item 7309 wherein the agent is not sirolimus.

7388. The method of item 7309 wherein the agent is everolimus or an analogue or derivative thereof.

7389. The method of item 7309 wherein the agent is tacrolimus or an analogue or derivative thereof.

7390. The method of item 7309 wherein the agent is not tacrolimus.

7391. The method of item 7309 wherein the agent is biolmus or an analogue or derivative thereof.

7392. The method of item 7309 wherein the agent is tresperimus or an analogue or derivative thereof.

7393. The method of item 7309 wherein the agent is auranofin or an analogue or derivative thereof.

7394. The method of item 7309 wherein the agent is 27-0-demethylrapamycin or an analogue or derivative thereof.

7395. The method of item 7309 wherein the agent is gusperimus or an analogue or derivative thereof.

7396. The method of item 7309 wherein the agent is pimecrolimus or an analogue or derivative thereof.

7397. The method of item 7309 wherein the agent is ABT-578 or an analogue or derivative thereof.

7398. The method of item 7309 wherein the agent is an inosine monophosphate dehydrogenase (IMPDH) inhibitor.

7399. The method of item 7309 wherein the agent is an IMPDH inhibitor, wherein the IMPDH inhibitor is mycophenolic acid or an analogue or derivative thereof.

7400. The method of item 7309 wherein the agent is an IMPDH inhibitor, wherein the IMPDH inhibitor is 1-alpha-25 dihydroxy vitamin D₃ or an analogue or derivative thereof.

7401. The method of item 7309 wherein the agent is a leukotriene inhibitor.

7402. The method of item 7309 wherein the agent is a MCP-1 antagonist.

7403. The method of item 7309 wherein the agent is a MMP inhibitor.

7404. The method of item 7309 wherein the agent is an NF kappa B inhibitor.

7405. The method of item 7309 wherein the agent is an NF kappa B inhibitor, wherein the NF kappa B inhibitor is Bay 11-7082.

7406. The method of item 7309 wherein the agent is an NO agonist.

7407. The method of item 7309 wherein the agent is a p38 MAP kinase inhibitor.

7408. The method of item 7309 wherein the agent is a p38 MAP kinase inhibitor, wherein the p38 MAP kinase inhibitor is SB 202190.

7409. The method of item 7309 wherein the agent is a phosphodiesterase inhibitor.

7410. The method of item 7309 wherein the agent is a TGF beta inhibitor.

7411. The method of item 7309 wherein the agent is a thromboxane A2 antagonist.

7412. The method of item 7309 wherein the agent is a TNFa antagonist.

7413. The method of item 7309 wherein the agent is a TACE inhibitor.

7414. The method of item 7309 wherein the agent is a tyrosine kinase inhibitor.

7415. The method of item 7309 wherein the agent is a vitronectin inhibitor.

7416. The method of item 7309 wherein the agent is a fibroblast growth factor inhibitor.

7417. The method of item 7309 wherein the agent is a protein kinase inhibitor.

7418. The method of item 7309 wherein the agent is a PDGF receptor kinase inhibitor.

7419. The method of item 7309 wherein the agent is an endothelial growth factor receptor kinase inhibitor.

7420. The method of item 7309 wherein the agent is a retinoic acid receptor antagonist.

7421. The method of item 7309 wherein the agent is a platelet derived growth factor receptor kinase inhibitor.

7422. The method of item 7309 wherein the agent is a fibronogin antagonist.

7423. The method of item 7309 wherein the agent is an antimycotic agent.

7424. The method of item 7309 wherein the agent is an antimycotic agent, wherein the antimycotic agent is sulconizole.

7425. The method of item 7309 wherein the agent is a bisphosphonate.

7426. The method of item 7309 wherein the agent is a phospholipase A1 inhibitor.

7427. The method of item 7309 wherein the agent is a histamine H1/H2/H3 receptor antagonist.

7428. The method of item 7309 wherein the agent is a macrolide antibiotic.

7429. The method of item 7309 wherein the agent is a GPIIb/IIIa receptor antagonist.

7430. The method of item 7309 wherein the agent is an endothelin receptor antagonist.

7431. The method of item 7309 wherein the agent is a peroxisome proliferator-activated receptor agonist.

7432. The method of item 7309 wherein the agent is an estrogen receptor agent.

7433. The method of item 7309 wherein the agent is a somastostatin analogue.

7434. The method of item 7309 wherein the agent is a neurokinin 1 antagonist.

7435. The method of item 7309 wherein the agent is a neurokinin 3 antagonist.

7436. The method of item 7309 wherein the agent is a VLA-4 antagonist.

7437. The method of item 7309 wherein the agent is an osteoclast inhibitor.

7438. The method of item 7309 wherein the agent is a DNA topoisomerase ATP hydrolyzing inhibitor.

7439. The method of item 7309 wherein the agent is an angiotensin I converting enzyme inhibitor.

7440. The method of item 7309 wherein the agent is an angiotensin II antagonist.

7441. The method of item 7309 wherein the agent is an enkephalinase inhibitor.

7442. The method of item 7309 wherein the agent is a peroxisome proliferator-activated receptor gamma agonist insulin sensitizer.

7443. The method of item 7309 wherein the agent is a protein kinase C inhibitor.

7444. The method of item 7309 wherein the agent is a ROCK (rho-associated kinase) inhibitor.

7445. The method of item 7309 wherein the agent is a CXCR3 inhibitor.

7446. The method of item 7309 wherein the agent is an Itk inhibitor.

7447. The method of item 7309 wherein the agent is a cytosolic phospholipase A₂-alpha inhibitor.

7448. The method of item 7309 wherein the agent is a PPAR agonist.

7449. The method of item 7309 wherein the agent is an immunosuppressant.

7450. The method of item 7309 wherein the agent is an Erb inhibitor.

7451. The method of item 7309 wherein the agent is an apoptosis agonist.

7452. The method of item 7309 wherein the agent is a lipocortin agonist.

7453. The method of item 7309 wherein the agent is a VCAM-1 antagonist.

7454. The method of item 7309 wherein the agent is a collagen antagonist.

7455. The method of item 7309 wherein the agent is an alpha 2 integrin antagonist.

7456. The method of item 7309 wherein the agent is a TNF alpha inhibitor.

7457. The method of item 7309 wherein the agent is a nitric oxide inhibitor.

7458. The method of item 7309 wherein the agent is a cathepsin inhibitor.

7459. The method of item 7309 wherein the agent is not an anti-inflammatory agent.

7460. The method of item 7309 wherein the agent is not a steroid.

7461. The method of item 7309 wherein the agent is not a glucocorticosteroid.

7462. The method of item 7309 wherein the agent is not dexamethasone.

7463. The method of item 7309 wherein the agent is not an anti-infective agent.

7464. The method of item 7309 wherein the agent is not an antibiotic.

7465. The method of item 7309 wherein the agent is not an anti-fungal agent.

7466. The method of item 7309, wherein the composition comprises a polymer.

7467. The method of item 7309, wherein the composition comprises a polymer, and the polymer is, or comprises, a copolymer.

7468. The method of item 7309, wherein the composition comprises a polymer, and the polymer is, or comprises, a block copolymer.

7469. The method of item 7309, wherein the composition comprises a polymer, and the polymer is, or comprises, a random copolymer.

7470. The method of item 7309, wherein the composition comprises a polymer, and the polymer is, or comprises, a biodegradable polymer.

7471. The method of item 7309, wherein the composition comprises a polymer, and the polymer is, or comprises, a non-biodegradable polymer.

7472. The method of item 7309, wherein the composition comprises a polymer, and the polymer is, or comprises, a hydrophilic polymer.

7473. The method of item 7309, wherein the composition comprises a polymer, and the polymer is, or comprises, a hydrophobic polymer.

7474. The method of item 7309, wherein the composition comprises a polymer, and the polymer is, or comprises, a polymer having hydrophilic domains.

7475. The method of item 7309, wherein the composition comprises a polymer, and the polymer is, or comprises, a polymer having hydrophobic domains.

7476. The method of item 7309, wherein the composition comprises a polymer, and the polymer is, or comprises, a non-conductive polymer.

7477. The method of item 7309, wherein the composition comprises a polymer, and the polymer is, or comprises, an elastomer.

7478. The method of item 7309, wherein the composition comprises a polymer, and the polymer is, or comprises, a hydrogel.

7479. The method of item 7309, wherein the composition comprises a polymer, and the polymer is, or comprises, a silicone polymer.

7480. The method of item 7309, wherein the composition comprises a polymer, and the polymer is, or comprises, a hydrocarbon polymer.

7481. The method of item 7309, wherein the composition comprises a polymer, and the polymer is, or comprises, a styrene-derived polymer.

7482. The method of item 7309, wherein the composition comprises a polymer, and the polymer is, or comprises, a butadiene-derived polymer.

7483. The method of item 7309, wherein the composition comprises a polymer, and the polymer is, or comprises, a macromer.

7484. The method of item 7309, wherein the composition comprises a polymer, and the polymer is, or comprises, a poly(ethylene glycol)polymer.

7485. The method of item 7309, wherein the composition comprises a polymer, and the polymer is, or comprises, an amorphous polymer.

7486. The method of item 7309, wherein the composition further comprises a second pharmaceutically active agent.

7487. The method of item 7309, wherein the composition further comprises an anti-inflammatory agent.

7488. The method of item 7309, wherein the composition further comprises an agent that inhibits infection.

7489. The method of item 7309, wherein the composition further comprises an anthracycline.

7490. The method of item 7309, wherein the composition further comprises doxorubicin.

7491. The method of item 7309 wherein the composition further comprises mitoxantrone.

7492. The method of item 7309 wherein the composition further comprises a fluoropyrimidine.

7493. The method of item 7309, wherein the composition further comprises 5-fluorouracil (5-FU).

7494. The method of item 7309, wherein the composition further comprises a folic acid antagonist.

7495. The method of item 7309, wherein the composition further comprises methotrexate.

7496. The method of item 7309, wherein the composition further comprises a podophylotoxin.

7497. The method of item 7309, wherein the composition further comprises etoposide.

7498. The method of item 7309, wherein the composition further comprises camptothecin.

7499. The method of item 7309, wherein the composition further comprises a hydroxyurea.

7500. The method of item 7309, wherein the composition further comprises a platinum complex.

7501. The method of item 7309, wherein the composition further comprises cisplatin.

7502. The method of item 7309 wherein the composition further comprises an anti-thrombotic agent.

7503. The method of item 7309, wherein the composition further comprises a visualization agent.

7504. The method of item 7309, wherein the composition further comprises a visualization agent, and the visualization agent is a radiopaque material, wherein the radiopaque material comprises a metal, a halogenated compound, or a barium containing compound.

7505. The method of item 7309, wherein the composition further comprises a visualization agent, and the visualization agent is, or comprises, barium, tantalum, or technetium.

7506. The method of item 7309, wherein the composition further comprises a visualization agent, and the visualization agent is, or comprises, an MRI responsive material.

7507. The method of item 7309, wherein the composition further comprises a visualization agent, and the visualization agent is, or comprises, a gadolinium chelate.

7508. The method of item 7309, wherein the composition further comprises a visualization agent, and the visualization agent is, or comprises, iron, magnesium, manganese, copper, or chromium.

7509. The method of item 7309, wherein the composition further comprises a visualization agent, and the visualization agent is, or comprises, iron oxide compound.

7510. The method of item 7309, wherein the composition further comprises a visualization agent, and the visualization agent is, or comprises, a dye, pigment, or colorant.

7511. The method of item 7309 wherein the agent is released in effective concentrations from the composition comprising the agent by diffusion over a period ranging from the time of administration to about 90 days.

7512. The method of item 7309 wherein the agent is released in effective concentrations from the composition comprising the agent by erosion of the composition over a period ranging from the time of administration to about 90 days.

7513. The method of item 7309 wherein the composition further comprises an inflammatory cytokine.

7514. The method of item 7309 wherein the composition further comprises an agent that stimulates cell proliferation.

7515. The method of item 7309 wherein the composition further comprises a polymeric carrier.

7516. The method of item 7309 wherein the composition is in the form of a gel, paste, or spray.

7517. The method of item 7309 wherein the implant is partially constructed with the agent or the composition.

7518. The method of item 7309 wherein the implant is fully constructed with the agent or the composition.

7519. The method of item 7309 wherein the implant is impregnated with the agent or the composition.

7520. The method of item 7309, wherein the agent or the composition forms a coating, and the coating directly contacts the implant.

7521. The method of item 7309, wherein the agent or the composition forms a coating, and the coating indirectly contacts the implant.

7522. The method of item 7309 wherein the agent or the composition forms a coating, and the coating partially covers the implant.

7523. The method of item 7309, wherein the agent or the composition forms a coating, and the coating completely covers the implant.

7524. The method of item 7309 wherein the agent or the composition is located within pores or holes of the implant.

7525. The method of item 7309 wherein the agent or the composition is located within a channel, lumen, or divet of the implant.

7526. The method of item 7309 wherein the implant further comprising an echogenic material.

7527. The method of item 7309 wherein the implant further comprises an echogenic material, wherein the echogenic material is in the form of a coating.

7528. The method of item 7309 wherein the implant is sterile.

7529. The method of item 7309 wherein the agent is delivered from the implant, wherein the agent is released into tissue in the vicinity of the implant after deployment of the implant.

7530. The method of item 7309 wherein the agent is delivered from the implant, wherein the agent is released into tissue in the vicinity of the implant after deployment of the implant, wherein the tissue is connective tissue.

7531. The method of item 7309 wherein the agent is delivered from the implant, wherein the agent is released into tissue in the vicinity of the implant after deployment of the implant, wherein the tissue is muscle tissue.

7532. The method of item 7309 wherein the agent is delivered from the implant, wherein the agent is released into tissue in the vicinity of the implant after deployment of the implant, wherein the tissue is nerve tissue.

7533. The method of item 7309 wherein the agent is delivered from the implant, wherein the agent is released into tissue in the vicinity of the implant after deployment of the implant, wherein the tissue is epithelium tissue.

7534. The method of item 7309 wherein the agent is delivered from the implant, wherein the agent is released in effective concentrations from the implant over a period ranging from the time of deployment of the implant to about 1 year.

7535. The method of item 7309 wherein the agent is delivered from the implant, wherein the agent is released in effective concentrations from the implant over a period ranging from about 1 month to 6 months.

7536. The method of item 7309 wherein the agent is delivered from the implant, wherein the agent is released in effective concentrations from the implant over a period ranging from about 1-90 days.

7537. The method of item 7309 wherein the agent is delivered from the implant, wherein the agent is released in effective concentrations from the implant at a constant rate.

7538. The method of item 7309 wherein the agent is delivered from the implant, wherein the agent is released in effective concentrations from the implant at an increasing rate.

7539. The method of item 7309 wherein the agent is delivered from the implant, wherein the agent is released in effective concentrations from the implant at a decreasing rate.

7540. The method of item 7309 wherein the agent is delivered from the implant, wherein the implant comprises about 0.01 μg to about 10 μg of the agent.

7541. The method of item 7309 wherein the agent is delivered from the implant, wherein the implant comprises about 10 μg to about 10 mg of the agent.

7542. The method of item 7309 wherein the agent is delivered from the implant, wherein the implant comprises about 10 mg to about 250 mg of the agent.

7543. The method of item 7309 wherein the agent is delivered from the implant, wherein the implant comprises about 250 mg to about 1000 mg of the agent.

7544. The method of item 7309 Wherein the agent is delivered from the implant, wherein the implant comprises about 1000 mg to about 2500 mg of the agent.

7545. The method of item 7309 wherein the agent is delivered from the implant, wherein a surface of the implant comprises less than 0.01 μg of the agent per mm² of implant surface to which the agent is applied.

7546. The method of item 7309 wherein the agent is delivered from the implant, wherein a surface of the implant comprises about 0.01 μg to about 1 μg of the agent per mm² of implant surface to which the agent is applied.

7547. The method of item 7309 wherein the agent is delivered from the implant, wherein a surface of the implant comprises about 1 μg to about 10 μg of the agent per mm2 of implant surface to which the agent is applied.

7548. The method of item 7309 wherein the agent is delivered from the implant, wherein a surface of the implant comprises about 10 μg to about 250 μg of the agent per mm² of implant surface to which the agent is applied.

7549. The method of item 7309 wherein the agent is delivered from the implant, wherein a surface of the implant comprises about 250 μg to about 1000 μg of the agent per mm2 of implant surface to which the agent is applied.

7550. The method of item 7309 wherein the agent is delivered from the implant, wherein a surface of the implant comprises about 1000 μg to about 2500 μg of the agent per mm2 of implant surface to which the agent is applied.

7551. The method of item 7309, wherein the implant further comprises a coating, and the coating is a uniform coating.

7552. The method of item 7309, wherein the implant further comprises a coating, and the coating is a non-uniform coating.

7553. The method of item 7309, wherein the implant further comprises a coating, and the coating is a discontinuous coating.

7554. The method of item 7309, wherein the implant further comprises a coating, and the coating is a patterned coating.

7555. The method of item 7309, wherein the implant further comprises a coating, and the coating has a thickness of 100 μm or less.

7556. The method of item 7309, wherein the implant further comprises a coating, and the coating has a thickness of 10 μm or less.

7557. The method of item 7309, wherein the implant further comprises a coating, and the coating adheres to the surface of the implant upon deployment of the implant.

7558. The method of item 7309, wherein the implant further comprises a coating, and the coating is stable at room temperature for a period of at least 1 year.

7559. The method of item 7309, wherein the implant further comprises a coating, and the agent is present in the coating in an amount ranging between about 0.0001% to about 1% by weight.

7560. The method of item 7309, wherein the implant further comprises a coating, and the agent is present in the coating in an amount ranging between about 1% to about 10% by weight.

7561. The method of item 7309, wherein the implant further comprises a coating, and the agent is present in the coating in an amount ranging between about 10% to about 25% by weight.

7562. The method of item 7309, wherein the implant further comprises a coating, and the agent is present in the coating in an amount ranging between about 25% to about 70% by weight.

7563. The method of item 7309, wherein the implant further comprises a coating, and the coating comprises a polymer.

7564. The method of item 7309, wherein the implant comprises a first coating having a first composition and a second coating having a second composition.

7565. The method of item 7309, wherein the implant comprises a first coating having a first composition and a second coating having a second composition, wherein the first composition and the second composition are different.

7566. A method for inhibiting scarring comprising placing a central nervous system shunt (i.e., an implant) and an anti-scarring agent or a composition comprising an anti-scarring agent into an animal host, wherein the agent inhibits scarring.

7567. The method of item 7566 wherein the agent inhibits cell regeneration.

7568. The method of item 7566 wherein the agent inhibits angiogenesis.

7569. The method of item 7566 wherein the agent inhibits fibroblast migration.

7570. The method of item 7566 wherein the agent inhibits fibroblast proliferation.

7571. The method of item 7566 wherein the agent inhibits deposition of extracellular matrix.

7572. The method of item 7566 wherein the agent inhibits tissue remodeling.

7573. The method of item 7566 wherein the agent is an angiogenesis inhibitor.

7574. The method of item 7566 wherein the agent is a 5-lipoxygenase inhibitor or antagonist.

7575. The method of item 7566 wherein the agent is a chemokine receptor antagonist.

7576. The method of item 7566 wherein the agent is a cell cycle inhibitor.

7577. The method of item 7566 wherein the agent is a taxane.

7578. The method of item 7566 wherein the agent is an anti-microtubule agent.

7579. The method of item 7566 wherein the agent is paclitaxel.

7580. The method of item 7566 wherein the agent is not paclitaxel.

7581. The method of item 7566 wherein the agent is an analogue or derivative of paclitaxel.

7582. The method of item 7566 wherein the agent is a vinca alkaloid.

7583. The method of item 7566 wherein the agent is camptothecin or an analogue or derivative thereof.

7584. The method of item 7566 wherein the agent is a podophyllotoxin.

7585. The method of item 7566 wherein the agent is a podophyllotoxin, wherein the podophyllotoxin is etoposide or an analogue or derivative thereof.

7586. The method of item 7566 wherein the agent is an anthracycline.

7587. The method of item 7566 wherein the agent is an anthracycline, wherein the anthracycline is doxorubicin or an analogue or derivative thereof.

7588. The method of item 7566 wherein the agent is an anthracycline, wherein the anthracycline is mitoxantrone or an analogue or derivative thereof.

7589. The method of item 7566 wherein the agent is a platinum compound.

7590. The method of item 7566 wherein the agent is a nitrosourea.

7591. The method of item 7566 wherein the agent is a nitroimidazole.

7592. The method of item 7566 wherein the agent is a folic acid antagonist.

7593. The method of item 7566 wherein the agent is a cytidine analogue.

7594. The method of item 7566 wherein the agent is a pyrimidine analogue.

7595. The method of item 7566 wherein the agent is a fluoropyrimidine analogue.

7596. The method of item 7566 wherein the agent is a purine analogue.

7597. The method of item 7566 wherein the agent is a nitrogen mustard or an analogue or derivative thereof.

7598. The method of item 7566 wherein the agent is a hydroxyurea.

7599. The method of item 7566 wherein the agent is a mytomicin or an analogue or derivative thereof.

7600. The method of item 7566 wherein the agent is an alkyl sulfonate.

7601. The method of item 7566 wherein the agent is a benzamide or an analogue or derivative thereof.

7602. The method of item 7566 wherein the agent is a nicotinamide or an analogue or derivative thereof.

7603. The method of item 7566 wherein the agent is a halogenated sugar or an analogue or derivative thereof.

7604. The method of item 7566 wherein the agent is a DNA alkylating agent.

7605. The method of item 7566 wherein the agent is an anti-microtubule agent.

7606. The method of item 7566 wherein the agent is a topoisomerase inhibitor.

7607. The method of item 7566 wherein the agent is a DNA cleaving agent.

7608. The method of item 7566 wherein the agent is an antimetabolite.

7609. The method of item 7566 wherein the agent inhibits adenosine deaminase.

7610. The method of item 7566 wherein the agent inhibits purine ring synthesis.

7611. The method of item 7566 wherein the agent is a nucleotide interconversion inhibitor.

7612. The method of item 7566 wherein the agent inhibits dihydrofolate reduction.

7613. The method of item 7566 wherein the agent blocks thymidine monophosphate.

7614. The method of item 7566 wherein the agent causes DNA damage.

7615. The method of item 7566 wherein the agent is a DNA intercalation agent.

7616. The method of item 7566 wherein the agent is a RNA synthesis inhibitor.

7617. The method of item 7566 wherein the agent is a pyrimidine synthesis inhibitor.

7618. The method of item 7566 wherein the agent inhibits ribonucleotide synthesis or function.

7619. The method of item 7566 wherein the agent inhibits thymidine monophosphate synthesis or function.

7620. The method of item 7566 wherein the agent inhibits DNA synthesis.

7621. The method of item 7566 wherein the agent causes DNA adduct formation.

7622. The method of item 7566 wherein the agent inhibits protein synthesis.

7623. The method of item 7566 wherein the agent inhibits microtubule function.

7624. The method of item 7566 wherein the agent is a cyclin dependent protein kinase inhibitor.

7625. The method of item 7566 wherein the agent is an epidermal growth factor kinase inhibitor.

7626. The method of item 7566 wherein the agent is an elastase inhibitor.

7627. The method of item 7566 wherein the agent is a factor Xa inhibitor.

7628. The method of item 7566 wherein the agent is a farnesyltransferase inhibitor.

7629. The method of item 7566 wherein the agent is a fibrinogen antagonist.

7630. The method of item 7566 wherein the agent is a guanylate cyclase stimulant.

7631. The method of item 7566 wherein the agent is a heat shock protein 90 antagonist.

7632. The method of item 7566 wherein the agent is a heat shock protein 90 antagonist, wherein the heat shock protein 90 antagonist is geldanamycin or an analogue or derivative thereof.

7633. The method of item 7566 wherein the agent is a guanylate cyclase stimulant.

7634. The method of item 7566 wherein the agent is a HMGCoA reductase inhibitor.

7635. The method of item 7566 wherein the agent is a HMGCoA reductase inhibitor, wherein the HMGCoA reductase inhibitor is simvastatin or an analogue or derivative thereof.

7636. The method of item 7566 wherein the agent is a hydroorotate dehydrogenase inhibitor.

7637. The method of item 7566 wherein the agent is an IKK2 inhibitor.

7638. The method of item 7566 wherein the agent is an IL-1 antagonist.

7639. The method of item 7566 wherein the agent is an ICE antagonist.

7640. The method of item 7566 wherein the agent is an IRAK antagonist.

7641. The method of item 7566 wherein the agent is an IL-4 agonist.

7642. The method of item 7566 wherein the agent is an immunomodulatory agent.

7643. The method of item 7566 wherein the agent is sirolimus or an analogue or derivative thereof.

7644. The method of item 7566 wherein the agent is not sirolimus.

7645. The method of item 7566 wherein the agent is everolimus or an analogue or derivative thereof.

7646. The method of item 7566 wherein the agent is tacrolimus or an analogue or derivative thereof.

7647. The method of item 7566 wherein the agent is not tacrolimus.

7648. The method of item 7566 wherein the agent is biolmus or an analogue or derivative thereof.

7649. The method of item 7566 wherein the agent is tresperimus or an analogue or derivative thereof.

7650. The method of item 7566 wherein the agent is auranofin or an analogue or derivative thereof.

7651. The method of item 7566 wherein the agent is 27-0-demethylrapamycin or an analogue or derivative thereof.

7652. The method of item 7566 wherein the agent is gusperimus or an analogue or derivative thereof.

7653. The method of item 7566 wherein the agent is pimecrolimus or an analogue or derivative thereof.

7654. The method of item 7566 wherein the agent is ABT-578 or an analogue or derivative thereof.

7655. The method of item 7566 wherein the agent is an inosine monophosphate dehydrogenase (IMPDH) inhibitor.

7656. The method of item 7566 wherein the agent is an IMPDH inhibitor, wherein the IMPDH inhibitor is mycophenolic acid or an analogue or derivative thereof.

7657. The method of item 7566 wherein the agent is an IMPDH inhibitor, wherein the IMPDH inhibitor is 1-alpha-25 dihydroxy vitamin D₃ or an analogue or derivative thereof.

7658. The method of item 7566 wherein the agent is a leukotriene inhibitor.

7659. The method of item 7566 wherein the agent is a MCP-1 antagonist.

7660. The method of item 7566 wherein the agent is a MMP inhibitor.

7661. The method of item 7566 wherein the agent is an NF kappa B inhibitor.

7662. The method of item 7566 wherein the agent is an NF kappa B inhibitor, wherein the NF kappa B inhibitor is Bay 11-7082.

7663. The method of item 7566 wherein the agent is an NO agonist.

7664. The method of item 7566 wherein the agent is a p38 MAP kinase inhibitor.

7665. The method of item 7566 wherein the agent is a p38 MAP kinase inhibitor, wherein the p38 MAP kinase inhibitor is SB 202190.

7666. The method of item 7566 wherein the agent is a phosphodiesterase inhibitor.

7667. The method of item 7566 wherein the agent is a TGF beta inhibitor.

7668. The method of item 7566 wherein the agent is a thromboxane A2 antagonist.

7669. The method of item 7566 wherein the agent is a TNFa antagonist.

7670. The method of item 7566 wherein the agent is a TACE inhibitor.

7671. The method of item 7566 wherein the agent is a tyrosine kinase inhibitor.

7672. The method of item 7566 wherein the agent is a vitronectin inhibitor.

7673. The method of item 7566 wherein the agent is a fibroblast growth factor inhibitor.

7674. The method of item 7566 wherein the agent is a protein kinase inhibitor.

7675. The method of item 7566 wherein the agent is a PDGF receptor kinase inhibitor.

7676. The method of item 7566 wherein the agent is an endothelial growth factor receptor kinase inhibitor.

7677. The method of item 7566 wherein the agent is a retinoic acid receptor antagonist.

7678. The method of item 7566 wherein the agent is a platelet derived growth factor receptor kinase inhibitor.

7679. The method of item 7566 wherein the agent is a fibronogin antagonist.

7680. The method of item 7566 wherein the agent is an antimycotic agent.

7681. The method of item 7566 wherein the agent is an antimycotic agent, wherein the antimycotic agent is sulconizole.

7682. The method of item 7566 wherein the agent is a bisphosphonate.

7683. The method of item 7566 wherein the agent is a phospholipase A1 inhibitor.

7684. The method of item 7566 wherein the agent is a histamine H1/H2/H3 receptor antagonist.

7685. The method of item 7566 wherein the agent is a macrolide antibiotic.

7686. The method of item 7566 wherein the agent is a GPIIb/IIIa receptor antagonist.

7687. The method of item 7566 wherein the agent is an endothelin receptor antagonist.

7688. The method of item 7566 wherein the agent is a peroxisome proliferator-activated receptor agonist.

7689. The method of item 7566 wherein the agent is an estrogen receptor agent.

7690. The method of item 7566 wherein the agent is a somastostatin analogue.

7691. The method of item 7566 wherein the agent is a neurokinin 1 antagonist.

7692. The method of item 7566 wherein the agent is a neurokinin 3 antagonist.

7693. The method of item 7566 wherein the agent is a VLA-4 antagonist.

7694. The method of item 7566 wherein the agent is an osteoclast inhibitor.

7695. The method of item 7566 wherein the agent is a DNA topoisomerase ATP hydrolyzing inhibitor.

7696. The method of item 7566 wherein the agent is an angiotensin I converting enzyme inhibitor.

7697. The method of item 7566 wherein the agent is an angiotensin II antagonist.

7698. The method of item 7566 wherein the agent is an enkephalinase inhibitor.

7699. The method of item 7566 wherein the agent is a peroxisome proliferator-activated receptor gamma agonist insulin sensitizer.

7700. The method of item 7566 wherein the agent is a protein kinase C inhibitor.

7701. The method of item 7566 wherein the agent is a ROCK (rho-associated kinase) inhibitor.

7702. The method of item 7566 wherein the agent is a CXCR3 inhibitor.

7703. The method of item 7566 wherein the agent is an Itk inhibitor.

7704. The method of item 7566 wherein the agent is a cytosolic phospholipase A₂-alpha inhibitor.

7705. The method of item 7566 wherein the agent is a PPAR agonist.

7706. The method of item 7566 wherein the agent is an immunosuppressant.

7707. The method of item 7566 wherein the agent is an Erb inhibitor.

7708. The method of item 7566 wherein the agent is an apoptosis agonist.

7709. The method of item 7566 wherein the agent is a lipocortin agonist.

7710. The method of item 7566 wherein the agent is a VCAM-1 antagonist.

7711. The method of item 7566 wherein the agent is a collagen antagonist.

7712. The method of item 7566 wherein the agent is an alpha 2 integrin antagonist.

7713. The method of item 7566 wherein the agent is a TNF alpha inhibitor.

7714. The method of item 7566 wherein the agent is a nitric oxide inhibitor.

7715. The method of item 7566 wherein the agent is a cathepsin inhibitor.

7716. The method of item 7566 wherein the agent is not an anti-inflammatory agent.

7717. The method of item 7566 wherein the agent is not a steroid.

7718. The method of item 7566 wherein the agent is not a glucocorticosteroid.

7719. The method of item 7566 wherein the agent is not dexamethasone.

7720. The method of item 7566 wherein the agent is not an anti-infective agent.

7721. The method of item 7566 wherein the agent is not an antibiotic.

7722. The method of item 7566 wherein the agent is not an anti-fungal agent.

7723. The method of item 7566, wherein the composition comprises a polymer.

7724. The method of item 7566, wherein the composition comprises a polymer, and the polymer is, or comprises, a copolymer.

7725. The method of item 7566, wherein the composition comprises a polymer, and the polymer is, or comprises, a block copolymer.

7726. The method of item 7566, wherein the composition comprises a polymer, and the polymer is, or comprises, a random copolymer.

7727. The method of item 7566, wherein the composition comprises a polymer, and the polymer is, or comprises, a biodegradable polymer.

7728. The method of item 7566, wherein the composition comprises a polymer, and the polymer is, or comprises, a non-biodegradable polymer.

7729. The method of item 7566, wherein the composition comprises a polymer, and the polymer is, or comprises, a hydrophilic polymer.

7730. The method of item 7566, wherein the composition comprises a polymer, and the polymer is, or comprises, a hydrophobic polymer.

7731. The method of item 7566, wherein the composition comprises a polymer, and the polymer is, or comprises, a polymer having hydrophilic domains.

7732. The method of item 7566, wherein the composition comprises a polymer, and the polymer is, or comprises, a polymer having hydrophobic domains.

7733. The method of item 7566, wherein the composition comprises a polymer, and the polymer is, or comprises, a non-conductive polymer.

7734. The method of item 7566, wherein the composition comprises a polymer, and the polymer is, or comprises, an elastomer.

7735. The method of item 7566, wherein the composition comprises a polymer, and the polymer is, or comprises, a hydrogel.

7736. The method of item 7566, wherein the composition comprises a polymer, and the polymer is, or comprises, a silicone polymer.

7737. The method of item 7566, wherein the composition comprises a polymer, and the polymer is, or comprises, a hydrocarbon polymer.

7738. The method of item 7566, wherein the composition comprises a polymer, and the polymer is, or comprises, a styrene-derived polymer.

7739. The method of item 7566, wherein the composition comprises a polymer, and the polymer is, or comprises, a butadiene-derived polymer.

7740. The method of item 7566, wherein the composition comprises a polymer, and the polymer is, or comprises, a macromer.

7741. The method of item 7566, wherein the composition comprises a polymer, and the polymer is, or comprises, a poly(ethylene glycol)polymer.

7742. The method of item 7566, wherein the composition comprises a polymer, and the polymer is, or comprises, an amorphous polymer.

7743. The method of item 7566, wherein the composition further comprises a second pharmaceutically active agent.

7744. The method of item 7566, wherein the composition further comprises an anti-inflammatory agent.

7745. The method of item 7566, wherein the composition further comprises an agent that inhibits infection.

7746. The method of item 7566, wherein the composition further comprises an anthracycline.

7747. The method of item 7566, wherein the composition further comprises doxorubicin.

7748. The method of item 7566 wherein the composition further comprises mitoxantrone.

7749. The method of item 7566 wherein the composition further comprises a fluoropyrimidine.

7750. The method of item 7566, wherein the composition further comprises 5-fluorouracil (5-FU).

7751. The method of item 7566, wherein the composition further comprises a folic acid antagonist.

7752. The method of item 7566, wherein the composition further comprises methotrexate.

7753. The method of item 7566, wherein the composition further comprises a podophylotoxin.

7754. The method of item 7566, wherein the composition further comprises etoposide.

7755. The method of item 7566, wherein the composition further comprises camptothecin.

7756. The method of item 7566, wherein the composition further comprises a hydroxyurea.

7757. The method of item 7566, wherein the composition further comprises a platinum complex.

7758. The method of item 7566, wherein the composition further comprises cisplatin.

7759. The method of item 7566 wherein the composition further comprises an anti-thrombotic agent.

7760. The method of item 7566, wherein the composition further comprises a visualization agent.

7761. The method of item 7566, wherein the composition further comprises a visualization agent, and the visualization agent is a radiopaque material, wherein the radiopaque material comprises a metal, a halogenated compound, or a barium containing compound.

7762. The method of item 7566, wherein the composition further comprises a visualization agent, and the visualization agent is, or comprises, barium, tantalum, or technetium.

7763. The method of item 7566, wherein the composition further comprises a visualization agent, and the visualization agent is, or comprises, an MRI responsive material.

7764. The method of item 7566, wherein the composition further comprises a visualization agent, and the visualization agent is, or comprises, a gadolinium chelate.

7765. The method of item 7566, wherein the composition further comprises a visualization agent, and the visualization agent is, or comprises, iron, magnesium, manganese, copper, or chromium.

7766. The method of item 7566, wherein the composition further comprises a visualization agent, and the visualization agent is, or comprises, iron oxide compound.

7767. The method of item 7566, wherein the composition further comprises a visualization agent, and the visualization agent is, or comprises, a dye, pigment, or colorant.

7768. The method of item 7566 wherein the agent is released in effective concentrations from the composition comprising the agent by diffusion over a period ranging from the time of administration to about 90 days.

7769. The method of item 7566 wherein the agent is released in effective concentrations from the composition comprising the agent by erosion of the composition over a period ranging from the time of administration to about 90 days.

7770. The method of item 7566 wherein the composition further comprises an inflammatory cytokine.

7771. The method of item 7566 wherein the composition further comprises an agent that stimulates cell proliferation.

7772. The method of item 7566 wherein the composition further comprises a polymeric carrier.

7773. The method of item 7566 wherein the composition is in the form of a gel, paste, or spray.

7774. The method of item 7566 wherein the implant is partially constructed with the agent or the composition.

7775. The method of item 7566 wherein the implant is fully constructed with the agent or the composition.

7776. The method of item 7566 wherein the implant is impregnated with the agent or the composition.

7777. The method of item 7566, wherein the agent or the composition forms a coating, and the coating directly contacts the implant.

7778. The method of item 7566, wherein the agent or the composition forms a coating, and the coating indirectly contacts the implant.

7779. The method of item 7566 wherein the agent or the composition forms a coating, and the coating partially covers the implant.

7780. The method of item 7566, wherein the agent or the composition forms a coating, and the coating completely covers the implant.

7781. The method of item 7566 wherein the agent or the composition is located within pores or holes of the implant.

7782. The method of item 7566 wherein the agent or the composition is located within a channel, lumen, or divet of the implant.

7783. The method of item 7566 wherein the implant further comprising an echogenic material.

7784. The method of item 7566 wherein the implant further comprises an echogenic material, wherein the echogenic material is in the form of a coating.

7785. The method of item 7566 wherein the implant is sterile.

7786. The method of item 7566 wherein the agent is delivered from the implant, wherein the agent is released into tissue in the vicinity of the implant after deployment of the implant.

7787. The method of item 7566 wherein the agent is delivered from the implant, wherein the agent is released into tissue in the vicinity of the implant after deployment of the implant, wherein the tissue is connective tissue.

7788. The method of item 7566 wherein the agent is delivered from the implant, wherein the agent is released into tissue in the vicinity of the implant after deployment of the implant, wherein the tissue is muscle tissue.

7789. The method of item 7566 wherein the agent is delivered from the implant, wherein the agent is released into tissue in the vicinity of the implant after deployment of the implant, wherein the tissue is nerve tissue.

7790. The method of item 7566 wherein the agent is delivered from the implant, wherein the agent is released into tissue in the vicinity of the implant after deployment of the implant, wherein the tissue is epithelium tissue.

7791. The method of item 7566 wherein the agent is delivered from the implant, wherein the agent is released in effective concentrations from the implant over a period ranging from the time of deployment of the implant to about 1 year.

7792. The method of item 7566 wherein the agent is delivered from the implant, wherein the agent is released in effective concentrations from the implant over a period ranging from about 1 month to 6 months.

7793. The method of item 7566 wherein the agent is delivered from the implant, wherein the agent is released in effective concentrations from the implant over a period ranging from about 1-90 days.

7794. The method of item 7566 wherein the agent is delivered from the implant, wherein the agent is released in effective concentrations from the implant at a constant rate.

7795. The method of item 7566 wherein the agent is delivered from the implant, wherein the agent is released in effective concentrations from the implant at an increasing rate.

7796. The method of item 7566 wherein the agent is delivered from the implant, wherein the agent is released in effective concentrations from the implant at a decreasing rate.

7797. The method of item 7566 wherein the agent is delivered from the implant, wherein the implant comprises about 0.01 μg to about 10 μg of the agent.

7798. The method of item 7566 wherein the agent is delivered from the implant, wherein the implant comprises about 10 μg to about 10 mg of the agent.

7799. The method of item 7566 wherein the agent is delivered from the implant, wherein the implant comprises about 10 mg to about 250 mg of the agent.

7800. The method of item 7566 wherein the agent is delivered from the implant, wherein the implant comprises about 250 mg to about 1000 mg of the agent.

7801. The method of item 7566 wherein the agent is delivered from the implant, wherein the implant comprises about 1000 mg to about 2500 mg of the agent.

7802. The method of item 7566 wherein the agent is delivered from the implant, wherein a surface of the implant comprises less than 0.01 μg of the agent per mm² of implant surface to which the agent is applied.

7803. The method of item 7566 wherein the agent is delivered from the implant, wherein a surface of the implant comprises about 0.01 μg to about 1 μg of the agent per mm² of implant surface to which the agent is applied.

7804. The method of item 7566 wherein the agent is delivered from the implant, wherein a surface of the implant comprises about 1 μg to about 10 μg of the agent per mm² of implant surface to which the agent is applied.

7805. The method of item 7566 wherein the agent is delivered from the implant, wherein a surface of the implant comprises about 10 μg to about 250 μg of the agent per mm² of implant surface to which the agent is applied.

7806. The method of item 7566 wherein the agent is delivered from the implant, wherein a surface of the implant comprises about 250 μg to about 1000 μg of the agent per mm² of implant surface to which the agent is applied.

7807. The method of item 7566 wherein the agent is delivered from the implant, wherein a surface of the implant comprises about 1000 μg to about 2500 μg of the agent per mm² of implant surface to which the agent is applied.

7808. The method of item 7566, wherein the implant further comprises a coating, and the coating is a uniform coating.

7809. The method of item 7566, wherein the implant further comprises a coating, and the coating is a non-uniform coating.

7810. The method of item 7566, wherein the implant further comprises a coating, and the coating is a discontinuous coating.

7811. The method of item 7566, wherein the implant further comprises a coating, and the coating is a patterned coating.

7812. The method of item 7566, wherein the implant further comprises a coating, and the coating has a thickness of 100 μm or less.

7813. The method of item 7566, wherein the implant further comprises a coating, and the coating has a thickness of 10 μm or less.

7814. The method of item 7566, wherein the implant further comprises a coating, and the coating adheres to the surface of the implant upon deployment of the implant.

7815. The method of item 7566, wherein the implant further comprises a coating, and the coating is stable at room temperature for a period of at least 1 year.

7816. The method of item 7566, wherein the implant further comprises a coating, and the agent is present in the coating in an amount ranging between about 0.0001% to about 1% by weight.

7817. The method of item 7566, wherein the implant further comprises a coating, and the agent is present in the coating in an amount ranging between about 1% to about 10% by weight.

7818. The method of item 7566, wherein the implant further comprises a coating, and the agent is present in the coating in an amount ranging between about 10% to about 25% by weight.

7819. The method of item 7566, wherein the implant further comprises a coating, and the agent is present in the coating in an amount ranging between about 25% to about 70% by weight.

7820. The method of item 7566, wherein the implant further comprises a coating, and the coating comprises a polymer.

7821. The method of item 7566, wherein the implant comprises a first coating having a first composition and a second coating having a second composition.

7822. The method of item 7566, wherein the implant comprises a first coating having a first composition and a second coating having a second composition, wherein the first composition and the second composition are different.

7823. A method for inhibiting scarring comprising placing an intraocular lens (i.e., an implant) and an anti-scarring agent or a composition comprising an anti-scarring agent into an animal host, wherein the agent inhibits scarring.

7824. The method of item 7823 wherein the agent inhibits cell regeneration.

7825. The method of item 7823 wherein the agent inhibits angiogenesis.

7826. The method of item 7823 wherein the agent inhibits fibroblast migration.

7827. The method of item 7823 wherein the agent inhibits fibroblast proliferation.

7828. The method of item 7823 wherein the agent inhibits deposition of extracellular matrix.

7829. The method of item 7823 wherein the agent inhibits tissue remodeling.

7830. The method of item 7823 wherein the agent is an angiogenesis inhibitor.

7831. The method of item 7823 wherein the agent is a 5-lipoxygenase inhibitor or antagonist.

7832. The method of item 7823 wherein the agent is a chemokine receptor antagonist.

7833. The method of item 7823 wherein the agent is a cell cycle inhibitor.

7834. The method of item 7823 wherein the agent is a taxane.

7835. The method of item 7823 wherein the agent is an anti-microtubule agent.

7836. The method of item 7823 wherein the agent is paclitaxel.

7837. The method of item 7823 wherein the agent is not paclitaxel.

7838. The method of item 7823 wherein the agent is an analogue or derivative of paclitaxel.

7839. The method of item 7823 wherein the agent is a vinca alkaloid.

7840. The method of item 7823 wherein the agent is camptothecin or an analogue or derivative thereof.

7841. The method of item 7823 wherein the agent is a podophyllotoxin.

7842. The method of item 7823 wherein the agent is a podophyllotoxin, wherein the podophyllotoxin is etoposide or an analogue or derivative thereof.

7843. The method of item 7823 wherein the agent is an anthracycline.

7844. The method of item 7823 wherein the agent is an anthracycline, wherein the anthracycline is doxorubicin or an analogue or derivative thereof.

7845. The method of item 7823 wherein the agent is an anthracycline, wherein the anthracycline is mitoxantrone or an analogue or derivative thereof.

7846. The method of item 7823 wherein the agent is a platinum compound.

7847. The method of item 7823 wherein the agent is a nitrosourea.

7848. The method of item 7823 wherein the agent is a nitroimidazole.

7849. The method of item 7823 wherein the agent is a folic acid antagonist.

7850. The method of item 7823 wherein the agent is a cytidine analogue.

7851. The method of item 7823 wherein the agent is a pyrimidine analogue.

7852. The method of item 7823 wherein the agent is a fluoropyrimidine analogue.

7853. The method of item 7823 wherein the agent is a purine analogue.

7854. The method of item 7823 wherein the agent is a nitrogen mustard or an analogue or derivative thereof.

7855. The method of item 7823 wherein the agent is a hydroxyurea.

7856. The method of item 7823 wherein the agent is a mytomicin or an analogue or derivative thereof.

7857. The method of item 7823 wherein the agent is an alkyl sulfonate.

7858. The method of item 7823 wherein the agent is a benzamide or an analogue or derivative thereof.

7859. The method of item 7823 wherein the agent is a nicotinamide or an analogue or derivative thereof.

7860. The method of item 7823 wherein the agent is a halogenated sugar or an analogue or derivative thereof.

7861. The method of item 7823 wherein the agent is a DNA alkylating agent.

7862. The method of item 7823 wherein the agent is an anti-microtubule agent.

7863. The method of item 7823 wherein the agent is a topoisomerase inhibitor.

7864. The method of item 7823 wherein the agent is a DNA cleaving agent.

7865. The method of item 7823 wherein the agent is an antimetabolite.

7866. The method of item 7823 wherein the agent inhibits adenosine deaminase.

7867. The method of item 7823 wherein the agent inhibits purine ring synthesis.

7868. The method of item 7823 wherein the agent is a nucleotide interconversion inhibitor.

7869. The method of item 7823 wherein the agent inhibits dihydrofolate reduction.

7870. The method of item 7823 wherein the agent blocks thymidine monophosphate.

7871. The method of item 7823 wherein the agent causes DNA damage.

7872. The method of item 7823 wherein the agent is a DNA intercalation agent.

7873. The method of item 7823 wherein the agent is a RNA synthesis inhibitor.

7874. The method of item 7823 wherein the agent is a pyrimidine synthesis inhibitor.

7875. The method of item 7823 wherein the agent inhibits ribonucleotide synthesis or function.

7876. The method of item 7823 wherein the agent inhibits thymidine monophosphate synthesis or function.

7877. The method of item 7823 wherein the agent inhibits DNA synthesis.

7878. The method of item 7823 wherein the agent causes DNA adduct formation.

7879. The method of item 7823 wherein the agent inhibits protein synthesis.

7880. The method of item 7823 wherein the agent inhibits microtubule function.

7881. The method of item 7823 wherein the agent is a cyclin dependent protein kinase inhibitor.

7882. The method of item 7823 wherein the agent is an epidermal growth factor kinase inhibitor.

7883. The method of item 7823 wherein the agent is an elastase inhibitor.

7884. The method of item 7823 wherein the agent is a factor Xa inhibitor.

7885. The method of item 7823 wherein the agent is a farnesyltransferase inhibitor.

7886. The method of item 7823 wherein the agent is a fibrinogen antagonist.

7887. The method of item 7823 wherein the agent is a guanylate cyclase stimulant.

7888. The method of item 7823 wherein the agent is a heat shock protein 90 antagonist.

7889. The method of item 7823 wherein the agent is a heat shock protein 90 antagonist, wherein the heat shock protein 90 antagonist is geldanamycin or an analogue or derivative thereof.

7890. The method of item 7823 wherein the agent is a guanylate cyclase stimulant.

7891. The method of item 7823 wherein the agent is a HMGCoA reductase inhibitor.

7892. The method of item 7823 wherein the agent is a HMGCoA reductase inhibitor, wherein the HMGCoA reductase inhibitor is simvastatin or an analogue or derivative thereof.

7893. The method of item 7823 wherein the agent is a hydroorotate dehydrogenase inhibitor.

7894. The method of item 7823 wherein the agent is an IKK2 inhibitor.

7895. The method of item 7823 wherein the agent is an IL-1 antagonist.

7896. The method of item 7823 wherein the agent is an ICE antagonist.

7897. The method of item 7823 wherein the agent is an IRAK antagonist.

7898. The method of item 7823 wherein the agent is an IL-4 agonist.

7899. The method of item 7823 wherein the agent is an immunomodulatory agent.

7900. The method of item 7823 wherein the agent is sirolimus or an analogue or derivative thereof.

7901. The method of item 7823 wherein the agent is not sirolimus.

7902. The method of item 7823 wherein the agent is everolimus or an analogue or derivative thereof.

7903. The method of item 7823 wherein the agent is tacrolimus or an analogue or derivative thereof.

7904. The method of item 7823 wherein the agent is not tacrolimus.

7905. The method of item 7823 wherein the agent is biolmus or an analogue or derivative thereof.

7906. The method of item 7823 wherein the agent is tresperimus or an analogue or derivative thereof.

7907. The method of item 7823 wherein the agent is auranofin or an analogue or derivative thereof.

7908. The method of item 7823 wherein the agent is 27-0-demethylrapamycin or an analogue or derivative thereof.

7909. The method of item 7823 wherein the agent is gusperimus or an analogue or derivative thereof.

7910. The method of item 7823 wherein the agent is pimecrolimus or an analogue or derivative thereof.

7911. The method of item 7823 wherein the agent is ABT-578 or an analogue or derivative thereof.

7912. The method of item 7823 wherein the agent is an inosine monophosphate dehydrogenase (IMPDH) inhibitor.

7913. The method of item 7823 wherein the agent is an IMPDH inhibitor, wherein the IMPDH inhibitor is mycophenolic acid or an analogue or derivative thereof.

7914. The method of item 7823 wherein the agent is an IMPDH inhibitor, wherein the IMPDH inhibitor is 1-alpha-25 dihydroxy vitamin D₃ or an analogue or derivative thereof.

7915. The method of item 7823 wherein the agent is a leukotriene inhibitor.

7916. The method of item 7823 wherein the agent is a MCP-1 antagonist.

7917. The method of item 7823 wherein the agent is a MMP inhibitor.

7918. The method of item 7823 wherein the agent is an NF kappa B inhibitor.

7919. The method of item 7823 wherein the agent is an NF kappa B inhibitor, wherein the NF kappa B inhibitor is Bay 11-7082.

7920. The method of item 7823 wherein the agent is an NO agonist.

7921. The method of item 7823 wherein the agent is a p38 MAP kinase inhibitor.

7922. The method of item 7823 wherein the agent is a p38 MAP kinase inhibitor, wherein the p38 MAP kinase inhibitor is SB 202190.

7923. The method of item 7823 wherein the agent is a phosphodiesterase inhibitor.

7924. The method of item 7823 wherein the agent is a TGF beta inhibitor.

7925. The method of item 7823 wherein the agent is a thromboxane A2 antagonist.

7926. The method of item 7823 wherein the agent is a TNFa antagonist.

7927. The method of item 7823 wherein the agent is a TACE inhibitor.

7928. The method of item 7823 wherein the agent is a tyrosine kinase inhibitor.

7929. The method of item 7823 wherein the agent is a vitronectin inhibitor.

7930. The method of item 7823 wherein the agent is a fibroblast growth factor inhibitor.

7931. The method of item 7823 wherein the agent is a protein kinase inhibitor.

7932. The method of item 7823 wherein the agent is a PDGF receptor kinase inhibitor.

7933. The method of item 7823 wherein the agent is an endothelial growth factor receptor kinase inhibitor.

7934. The method of item 7823 wherein the agent is a retinoic acid receptor antagonist.

7935. The method of item 7823 wherein the agent is a platelet derived growth factor receptor kinase inhibitor.

7936. The method of item 7823 wherein the agent is a fibronogin antagonist.

7937. The method of item 7823 wherein the agent is an antimycotic agent.

7938. The method of item 7823 wherein the agent is an antimycotic agent, wherein the antimycotic agent is sulconizole.

7939. The method of item 7823 wherein the agent is a bisphosphonate.

7940. The method of item 7823 wherein the agent is a phospholipase A1 inhibitor.

7941. The method of item 7823 wherein the agent is a histamine H1/H2/H3 receptor antagonist.

7942. The method of item 7823 wherein the agent is a macrolide antibiotic.

7943. The method of item 7823 wherein the agent is a GPIIb/IIIa receptor antagonist.

7944. The method of item 7823 wherein the agent is an endothelin receptor antagonist.

7945. The method of item 7823 wherein the agent is a peroxisome proliferator-activated receptor agonist.

7946. The method of item 7823 wherein the agent is an estrogen receptor agent.

7947. The method of item 7823 wherein the agent is a somastostatin analogue.

7948. The method of item 7823 wherein the agent is a neurokinin 1 antagonist.

7949. The method of item 7823 wherein the agent is a neurokinin 3 antagonist.

7950. The method of item 7823 wherein the agent is a VLA-4 antagonist.

7951. The method of item 7823 wherein the agent is an osteoclast inhibitor.

7952. The method of item 7823 wherein the agent is a DNA topoisomerase ATP hydrolyzing inhibitor.

7953. The method of item 7823 wherein the agent is an angiotensin I converting enzyme inhibitor.

7954. The method of item 7823 wherein the agent is an angiotensin II antagonist.

7955. The method of item 7823 wherein the agent is an enkephalinase inhibitor.

7956. The method of item 7823 wherein the agent is a peroxisome proliferator-activated receptor gamma agonist insulin sensitizer.

7957. The method of item 7823 wherein the agent is a protein kinase C inhibitor.

7958. The method of item 7823 wherein the agent is a ROCK (rho-associated kinase) inhibitor.

7959. The method of item 7823 wherein the agent is a CXCR3 inhibitor.

7960. The method of item 7823 wherein the agent is an Itk inhibitor.

7961. The method of item 7823 wherein the agent is a cytosolic phospholipase A₂-alpha inhibitor.

7962. The method of item 7823 wherein the agent is a PPAR agonist.

7963. The method of item 7823 wherein the agent is an immunosuppressant.

7964. The method of item 7823 wherein the agent is an Erb inhibitor.

7965. The method of item 7823 wherein the agent is an apoptosis agonist.

7966. The method of item 7823 wherein the agent is a lipocortin agonist.

7967. The method of item 7823 wherein the agent is a VCAM-1 antagonist.

7968. The method of item 7823 wherein the agent is a collagen antagonist.

7969. The method of item 7823 wherein the agent is an alpha 2 integrin antagonist.

7970. The method of item 7823 wherein the agent is a TNF alpha inhibitor.

7971. The method of item 7823 wherein the agent is a nitric oxide inhibitor.

7972. The method of item 7823 wherein the agent is a cathepsin inhibitor.

7973. The method of item 7823 wherein the agent is not an anti-inflammatory agent.

7974. The method of item 7823 wherein the agent is not a steroid.

7975. The method of item 7823 wherein the agent is not a glucocorticosteroid.

7976. The method of item 7823 wherein the agent is not dexamethasone.

7977. The method of item 7823 wherein the agent is not an anti-infective agent.

7978. The method of item 7823 wherein the agent is not an antibiotic.

7979. The method of item 7823 wherein the agent is not an anti-fungal agent.

7980. The method of item 7823, wherein the composition comprises a polymer.

7981. The method of item 7823, wherein the composition comprises a polymer, and the polymer is, or comprises, a copolymer.

7982. The method of item 7823, wherein the composition comprises a polymer, and the polymer is, or comprises, a block copolymer.

7983. The method of item 7823, wherein the composition comprises a polymer, and the polymer is, or comprises, a random copolymer.

7984. The method of item 7823, wherein the composition comprises a polymer, and the polymer is, or comprises, a biodegradable polymer.

7985. The method of item 7823, wherein the composition comprises a polymer, and the polymer is, or comprises, a non-biodegradable polymer.

7986. The method of item 7823, wherein the composition comprises a polymer, and the polymer is, or comprises, a hydrophilic polymer.

7987. The method of item 7823, wherein the composition comprises a polymer, and the polymer is, or comprises, a hydrophobic polymer.

7988. The method of item 7823, wherein the composition comprises a polymer, and the polymer is, or comprises, a polymer having hydrophilic domains.

7989. The method of item 7823, wherein the composition comprises a polymer, and the polymer is, or comprises, a polymer having hydrophobic domains.

7990. The method of item 7823, wherein the composition comprises a polymer, and the polymer is, or comprises, a non-conductive polymer.

7991. The method of item 7823, wherein the composition comprises a polymer, and the polymer is, or comprises, an elastomer.

7992. The method of item 7823, wherein the composition comprises a polymer, and the polymer is, or comprises, a hydrogel.

7993. The method of item 7823, wherein the composition comprises a polymer, and the polymer is, or comprises, a silicone polymer.

7994. The method of item 7823, wherein the composition comprises a polymer, and the polymer is, or comprises, a hydrocarbon polymer.

7995. The method of item 7823, wherein the composition comprises a polymer, and the polymer is, or comprises, a styrene-derived polymer.

7996. The method of item 7823, wherein the composition comprises a polymer, and the polymer is, or comprises, a butadiene-derived polymer.

7997. The method of item 7823, wherein the composition comprises a polymer, and the polymer is, or comprises, a macromer.

7998. The method of item 7823, wherein the composition comprises a polymer, and the polymer is, or comprises, a poly(ethylene glycol)polymer.

7999. The method of item 7823, wherein the composition comprises a polymer, and the polymer is, or comprises, an amorphous polymer.

8000. The method of item 7823, wherein the composition further comprises a second pharmaceutically active agent.

8001. The method of item 7823, wherein the composition further comprises an anti-inflammatory agent.

8002. The method of item 7823, wherein the composition further comprises an agent that inhibits infection.

8003. The method of item 7823, wherein the composition further comprises an anthracycline.

8004. The method of item 7823, wherein the composition further comprises doxorubicin.

8005. The method of item 7823 wherein the composition further comprises mitoxantrone.

8006. The method of item 7823 wherein the composition further comprises a fluoropyrimidine.

8007. The method of item 7823, wherein the composition further comprises 5-fluorouracil (5-FU).

8008. The method of item 7823, wherein the composition further comprises a folic acid antagonist.

8009. The method of item 7823, wherein the composition further comprises methotrexate.

8010. The method of item 7823, wherein the composition further comprises a podophylotoxin.

8011. The method of item 7823, wherein the composition further comprises etoposide.

8012. The method of item 7823, wherein the composition further comprises camptothecin.

8013. The method of item 7823, wherein the composition further comprises a hydroxyurea.

8014. The method of item 7823, wherein the composition further comprises a platinum complex.

8015. The method of item 7823, wherein the composition further comprises cisplatin.

8016. The method of item 7823 wherein the composition further comprises an anti-thrombotic agent.

8017. The method of item 7823, wherein the composition further comprises a visualization agent.

8018. The method of item 7823, wherein the composition further comprises a visualization agent, and the visualization agent is a radiopaque material, wherein the radiopaque material comprises a metal, a halogenated compound, or a barium containing compound.

8019. The method of item 7823, wherein the composition further comprises a visualization agent, and the visualization agent is, or comprises, barium, tantalum, or technetium.

8020. The method of item 7823, wherein the composition further comprises a visualization agent, and the visualization agent is, or comprises, an MRI responsive material.

8021. The method of item 7823, wherein the composition further comprises a visualization agent, and the visualization agent is, or comprises, a gadolinium chelate.

8022. The method of item 7823, wherein the composition further comprises a visualization agent, and the visualization agent is, or comprises, iron, magnesium, manganese, copper, or chromium.

8023. The method of item 7823, wherein the composition further comprises a visualization agent, and the visualization agent is, or comprises, iron oxide compound.

8024. The method of item 7823, wherein the composition further comprises a visualization agent, and the visualization agent is, or comprises, a dye, pigment, or colorant.

8025. The method of item 7823 wherein the agent is released in effective concentrations from the composition comprising the agent by diffusion over a period ranging from the time of administration to about 90 days.

8026. The method of item 7823 wherein the agent is released in effective concentrations from the composition comprising the agent by erosion of the composition over a period ranging from the time of administration to about 90 days.

8027. The method of item 7823 wherein the composition further comprises an inflammatory cytokine.

8028. The method of item 7823 wherein the composition further comprises an agent that stimulates cell proliferation.

8029. The method of item 7823 wherein the composition further comprises a polymeric carrier.

8030. The method of item 7823 wherein the composition is in the form of a gel, paste, or spray.

8031. The method of item 7823 wherein the implant is partially constructed with the agent or the composition.

8032. The method of item 7823 wherein the implant is fully constructed with the agent or the composition.

8033. The method of item 7823 wherein the implant is impregnated with the agent or the composition.

8034. The method of item 7823, wherein the agent or the composition forms a coating, and the coating directly contacts the implant.

8035. The method of item 7823, wherein the agent or the composition forms a coating, and the coating indirectly contacts the implant.

8036. The method of item 7823 wherein the agent or the composition forms a coating, and the coating partially covers the implant.

8037. The method of item 7823, wherein the agent or the composition forms a coating, and the coating completely covers the implant.

8038. The method of item 7823 wherein the agent or the composition is located within pores or holes of the implant.

8039. The method of item 7823 wherein the agent or the composition is located within a channel, lumen, or divet of the implant.

8040. The method of item 7823 wherein the implant further comprising an echogenic material.

8041. The method of item 7823 wherein the implant further comprises an echogenic material, wherein the echogenic material is in the form of a coating.

8042. The method of item 7823 wherein the implant is sterile.

8043. The method of item 7823 wherein the agent is delivered from the implant, wherein the agent is released into tissue in the vicinity of the implant after deployment of the implant.

8044. The method of item 7823 wherein the agent is delivered from the implant, wherein the agent is released into tissue in the vicinity of the implant after deployment of the implant, wherein the tissue is connective tissue.

8045. The method of item 7823 wherein the agent is delivered from the implant, wherein the agent is released into tissue in the vicinity of the implant after deployment of the implant, wherein the tissue is muscle tissue.

8046. The method of item 7823 wherein the agent is delivered from the implant, wherein the agent is released into tissue in the vicinity of the implant after deployment of the implant, wherein the tissue is nerve tissue.

8047. The method of item 7823 wherein the agent is delivered from the implant, wherein the agent is released into tissue in the vicinity of the implant after deployment of the implant, wherein the tissue is epithelium tissue.

8048. The method of item 7823 wherein the agent is delivered from the implant, wherein the agent is released in effective concentrations from the implant over a period ranging from the time of deployment of the implant to about 1 year.

8049. The method of item 7823 wherein the agent is delivered from the implant, wherein the agent is released in effective concentrations from the implant over a period ranging from about 1 month to 6 months.

8050. The method of item 7823 wherein the agent is delivered from the implant, wherein the agent is released in effective concentrations from the implant over a period ranging from about 1-90 days.

8051. The method of item 7823 wherein the agent is delivered from the implant, wherein the agent is released in effective concentrations from the implant at a constant rate.

8052. The method of item 7823 wherein the agent is delivered from the implant, wherein the agent is released in effective concentrations from the implant at an increasing rate.

8053. The method of item 7823 wherein the agent is delivered from the implant, wherein the agent is released in effective concentrations from the implant at a decreasing rate.

8054. The method of item 7823 wherein the agent is delivered from the implant, wherein the implant comprises about 0.01 μg to about 10 μg of the agent.

8055. The method of item 7823 wherein the agent is delivered from the implant, wherein the implant comprises about 10 μg to about 10 mg of the agent.

8056. The method of item 7823 wherein the agent is delivered from the implant, wherein the implant comprises about 10 mg to about 250 mg of the agent.

8057. The method of item 7823 wherein the agent is delivered from the implant, wherein the implant comprises about 250 mg to about 1000 mg of the agent.

8058. The method of item 7823 wherein the agent is delivered from the implant, wherein the implant comprises about 1000 mg to about 2500 mg of the agent.

8059. The method of item 7823 wherein the agent is delivered from the implant, wherein a surface of the implant comprises less than 0.01 μg of the agent per mm² of implant surface to which the agent is applied.

8060. The method of item 7823 wherein the agent is delivered from the implant, wherein a surface of the implant comprises about 0.01 μg to about 1 μg of the agent per mm² of implant surface to which the agent is applied.

8061. The method of item 7823 wherein the agent is delivered from the implant, wherein a surface of the implant comprises about 1 μg to about 10 μg of the agent per mm² of implant surface to which the agent is applied.

8062. The method of item 7823 wherein the agent is delivered from the implant, wherein a surface of the implant comprises about 10 μg to about 250 μg of the agent per mm² of implant surface to which the agent is applied.

8063. The method of item 7823 wherein the agent is delivered from the implant, wherein a surface of the implant comprises about 250 μg to about 1000 μg of the agent per mm² of implant surface to which the agent is applied.

8064. The method of item 7823 wherein the agent is delivered from the implant, wherein a surface of the implant comprises about 1000 μg to about 2500 μg of the agent per mm² of implant surface to which the agent is applied.

8065. The method of item 7823, wherein the implant further comprises a coating, and the coating is a uniform coating.

8066. The method of item 7823, wherein the implant further comprises a coating, and the coating is a non-uniform coating.

8067. The method of item 7823, wherein the implant further comprises a coating, and the coating is a discontinuous coating.

8068. The method of item 7823, wherein the implant further comprises a coating, and the coating is a patterned coating.

8069. The method of item 7823, wherein the implant further comprises a coating, and the coating has a thickness of 100 μm or less.

8070. The method of item 7823, wherein the implant further comprises a coating, and the coating has a thickness of 10 μm or less.

8071. The method of item 7823, wherein the implant further comprises a coating, and the coating adheres to the surface of the implant upon deployment of the implant.

8072. The method of item 7823, wherein the implant further comprises a coating, and the coating is stable at room temperature for a period of at least 1 year.

8073. The method of item 7823, wherein the implant further comprises a coating, and the agent is present in the coating in an amount ranging between about 0.0001% to about 1% by weight.

8074. The method of item 7823, wherein the implant further comprises a coating, and the agent is present in the coating in an amount ranging between about 1% to about 10% by weight.

8075. The method of item 7823, wherein the implant further comprises a coating, and the agent is present in the coating in an amount ranging between about 10% to about 25% by weight.

8076. The method of item 7823, wherein the implant further comprises a coating, and the agent is present in the coating in an amount ranging between about 25% to about 70% by weight.

8077. The method of item 7823, wherein the implant further comprises a coating, and the coating comprises a polymer.

8078. The method of item 7823, wherein the implant comprises a first coating having a first composition and a second coating having a second composition.

8079. The method of item 7823, wherein the implant comprises a first coating having a first composition and a second coating having a second composition, wherein the first composition and the second composition are different.

8080. A method for inhibiting scarring comprising placing a glaucoma drainage device (i.e., an implant) and an anti-scarring agent or a composition comprising an anti-scarring agent into an animal host, wherein the agent inhibits scarring.

8081. The method of item 8080 wherein the agent inhibits cell regeneration.

8082. The method of item 8080 wherein the agent inhibits angiogenesis.

8083. The method of item 8080 wherein the agent inhibits fibroblast migration.

8084. The method of item 8080 wherein the agent inhibits fibroblast proliferation.

8085. The method of item 8080 wherein the agent inhibits deposition of extracellular matrix.

8086. The method of item 8080 wherein the agent inhibits tissue remodeling.

8087. The method of item 8080 wherein the agent is an angiogenesis inhibitor.

8088. The method of item 8080 wherein the agent is a 5-lipoxygenase inhibitor or antagonist.

8089. The method of item 8080 wherein the agent is a chemokine receptor antagonist.

8090. The method of item 8080 wherein the agent is a cell cycle inhibitor.

8091. The method of item 8080 wherein the agent is a taxane.

8092. The method of item 8080 wherein the agent is an anti-microtubule agent.

8093. The method of item 8080 wherein the agent is paclitaxel.

8094. The method of item 8080 wherein the agent is not paclitaxel.

8095. The method of item 8080 wherein the agent is an analogue or derivative of paclitaxel.

8096. The method of item 8080 wherein the agent is a vinca alkaloid.

8097. The method of item 8080 wherein the agent is camptothecin or an analogue or derivative thereof.

8098. The method of item 8080 wherein the agent is a podophyllotoxin.

8099. The method of item 8080 wherein the agent is a podophyllotoxin, wherein the podophyllotoxin is etoposide or an analogue or derivative thereof.

8100. The method of item 8080 wherein the agent is an anthracycline.

8101. The method of item 8080 wherein the agent is an anthracycline, wherein the anthracycline is doxorubicin or an analogue or derivative thereof.

8102. The method of item 8080 wherein the agent is an anthracycline, wherein the anthracycline is mitoxantrone or an analogue or derivative thereof.

8103. The method of item 8080 wherein the agent is a platinum compound.

8104. The method of item 8080 wherein the agent is a nitrosourea.

8105. The method of item 8080 wherein the agent is a nitroimidazole.

8106. The method of item 8080 wherein the agent is a folic acid antagonist.

8107. The method of item 8080 wherein the agent is a cytidine analogue.

8108. The method of item 8080 wherein the agent is a pyrimidine analogue.

8109. The method of item 8080 wherein the agent is a fluoropyrimidine analogue.

8110. The method of item 8080 wherein the agent is a purine analogue.

8111. The method of item 8080 wherein the agent is a nitrogen mustard or an analogue or derivative thereof.

8112. The method of item 8080 wherein the agent is a hydroxyurea.

8113. The method of item 8080 wherein the agent is a mytomicin or an analogue or derivative thereof.

8114. The method of item 8080 wherein the agent is an alkyl sulfonate.

8115. The method of item 8080 wherein the agent is a benzamide or an analogue or derivative thereof.

8116. The method of item 8080 wherein the agent is a nicotinamide or an analogue or derivative thereof.

8117. The method of item 8080 wherein the agent is a halogenated sugar or an analogue or derivative thereof.

8118. The method of item 8080 wherein the agent is a DNA alkylating agent.

8119. The method of item 8080 wherein the agent is an anti-microtubule agent.

8120. The method of item 8080 wherein the agent is a topoisomerase inhibitor.

8121. The method of item 8080 wherein the agent is a DNA cleaving agent.

8122. The method of item 8080 wherein the agent is an antimetabolite.

8123. The method of item 8080 wherein the agent inhibits adenosine deaminase.

8124. The method of item 8080 wherein the agent inhibits purine ring synthesis.

8125. The method of item 8080 wherein the agent is a nucleotide interconversion inhibitor.

8126. The method of item 8080 wherein the agent inhibits dihydrofolate reduction.

8127. The method of item 8080 wherein the agent blocks thymidine monophosphate.

8128. The method of item 8080 wherein the agent causes DNA damage.

8129. The method of item 8080 wherein the agent is a DNA intercalation agent.

8130. The method of item 8080 wherein the agent is a RNA synthesis inhibitor.

8131. The method of item 8080 wherein the agent is a pyrimidine synthesis inhibitor.

8132. The method of item 8080 wherein the agent inhibits ribonucleotide synthesis or function.

8133. The method of item 8080 wherein the agent inhibits thymidine monophosphate synthesis or function.

8134. The method of item 8080 wherein the agent inhibits DNA synthesis.

8135. The method of item 8080 wherein the agent causes DNA adduct formation.

8136. The method of item 8080 wherein the agent inhibits protein synthesis.

8137. The method of item 8080 wherein the agent inhibits microtubule function.

8138. The method of item 8080 wherein the agent is a cyclin dependent protein kinase inhibitor.

8139. The method of item 8080 wherein the agent is an epidermal growth factor kinase inhibitor.

8140. The method of item 8080 wherein the agent is an elastase inhibitor.

8141. The method of item 8080 wherein the agent is a factor Xa inhibitor.

8142. The method of item 8080 wherein the agent is a farnesyltransferase inhibitor.

8143. The method of item 8080 wherein the agent is a fibrinogen antagonist.

8144. The method of item 8080 wherein the agent is a guanylate cyclase stimulant.

8145. The method of item 8080 wherein the agent is a heat shock protein 90 antagonist.

8146. The method of item 8080 wherein the agent is a heat shock protein 90 antagonist, wherein the heat shock protein 90 antagonist is geldanamycin or an analogue or derivative thereof.

8147. The method of item 8080 wherein the agent is a guanylate cyclase stimulant.

8148. The method of item 8080 wherein the agent is a HMGCoA reductase inhibitor.

8149. The method of item 8080 wherein the agent is a HMGCoA reductase inhibitor, wherein the HMGCoA reductase inhibitor is simvastatin or an analogue or derivative thereof.

8150. The method of item 8080 wherein the agent is a hydroorotate dehydrogenase inhibitor.

8151. The method of item 8080 wherein the agent is an IKK2 inhibitor.

8152. The method of item 8080 wherein the agent is an IL-1 antagonist.

8153. The method of item 8080 wherein the agent is an ICE antagonist.

8154. The method of item 8080 wherein the agent is an IRAK antagonist.

8155. The method of item 8080 wherein the agent is an IL-4 agonist.

8156. The method of item 8080 wherein the agent is an immunomodulatory agent.

8157. The method of item 8080 wherein the agent is sirolimus or an analogue or derivative thereof.

8158. The method of item 8080 wherein the agent is not sirolimus.

8159. The method of item 8080 wherein the agent is everolimus or an analogue or derivative thereof.

8160. The method of item 8080 wherein the agent is tacrolimus or an analogue or derivative thereof.

8161. The method of item 8080 wherein the agent is not tacrolimus.

8162. The method of item 8080 wherein the agent is biolmus or an analogue or derivative thereof.

8163. The method of item 8080 wherein the agent is tresperimus or an analogue or derivative thereof.

8164. The method of item 8080 wherein the agent is auranofin or an analogue or derivative thereof.

8165. The method of item 8080 wherein the agent is 27-0-demethylrapamycin or an analogue or derivative thereof.

8166. The method of item 8080 wherein the agent is gusperimus or an analogue or derivative thereof.

8167. The method of item 8080 wherein the agent is pimecrolimus or an analogue or derivative thereof.

8168. The method of item 8080 wherein the agent is ABT-578 or an analogue or derivative thereof.

8169. The method of item 8080 wherein the agent is an inosine monophosphate dehydrogenase (IMPDH) inhibitor.

8170. The method of item 8080 wherein the agent is an IMPDH inhibitor, wherein the IMPDH inhibitor is mycophenolic acid or an analogue or derivative thereof.

8171. The method of item 8080 wherein the agent is an IMPDH inhibitor, wherein the IMPDH inhibitor is 1-alpha-25 dihydroxy vitamin D₃ or an analogue or derivative thereof.

8172. The method of item 8080 wherein the agent is a leukotriene inhibitor.

8173. The method of item 8080 wherein the agent is a MCP-1 antagonist.

8174. The method of item 8080 wherein the agent is a MMP inhibitor.

8175. The method of item 8080 wherein the agent is an NF kappa B inhibitor.

8176. The method of item 8080 wherein the agent is an NF kappa B inhibitor, wherein the NF kappa B inhibitor is Bay 11-7082.

8177. The method of item 8080 wherein the agent is an NO agonist.

8178. The method of item 8080 wherein the agent is a p38 MAP kinase inhibitor.

8179. The method of item 8080 wherein the agent is a p38 MAP kinase inhibitor, wherein the p38 MAP kinase inhibitor is SB 202190.

8180. The method of item 8080 wherein the agent is a phosphodiesterase inhibitor.

8181. The method of item 8080 wherein the agent is a TGF beta inhibitor.

8182. The method of item 8080 wherein the agent is a thromboxane A2 antagonist.

8183. The method of item 8080 wherein the agent is a TNFa antagonist.

8184. The method of item 8080 wherein the agent is a TACE inhibitor.

8185. The method of item 8080 wherein the agent is a tyrosine kinase inhibitor.

8186. The method of item 8080 wherein the agent is a vitronectin inhibitor.

8187. The method of item 8080 wherein the agent is a fibroblast growth factor inhibitor.

8188. The method of item 8080 wherein the agent is a protein kinase inhibitor.

8189. The method of item 8080 wherein the agent is a PDGF receptor kinase inhibitor.

8190. The method of item 8080 wherein the agent is an endothelial growth factor receptor kinase inhibitor.

8191. The method of item 8080 wherein the agent is a retinoic acid receptor antagonist.

8192. The method of item 8080 wherein the agent is a platelet derived growth factor receptor kinase inhibitor.

8193. The method of item 8080 wherein the agent is a fibronogin antagonist.

8194. The method of item 8080 wherein the agent is an antimycotic agent.

8195. The method of item 8080 wherein the agent is an antimycotic agent, wherein the antimycotic agent is sulconizole.

8196. The method of item 8080 wherein the agent is a bisphosphonate.

8197. The method of item 8080 wherein the agent is a phospholipase A1 inhibitor.

8198. The method of item 8080 wherein the agent is a histamine H1/H2/H3 receptor antagonist.

8199. The method of item 8080 wherein the agent is a macrolide antibiotic.

8200. The method of item 8080 wherein the agent is a GPIIb/IIIa receptor antagonist.

8201. The method of item 8080 wherein the agent is an endothelin receptor antagonist.

8202. The method of item 8080 wherein the agent is a peroxisome proliferator-activated receptor agonist.

8203. The method of item 8080 wherein the agent is an estrogen receptor agent.

8204. The method of item 8080 wherein the agent is a somastostatin analogue.

8205. The method of item 8080 wherein the agent is a neurokinin 1 antagonist.

8206. The method of item 8080 wherein the agent is a neurokinin 3 antagonist.

8207. The method of item 8080 wherein the agent is a VLA-4 antagonist.

8208. The method of item 8080 wherein the agent is an osteoclast inhibitor.

8209. The method of item 8080 wherein the agent is a DNA topoisomerase ATP hydrolyzing inhibitor.

8210. The method of item 8080 wherein the agent is an angiotensin I converting enzyme inhibitor.

8211. The method of item 8080 wherein the agent is an angiotensin II antagonist.

8212. The method of item 8080 wherein the agent is an enkephalinase inhibitor.

8213. The method of item 8080 wherein the agent is a peroxisome proliferator-activated receptor gamma agonist insulin sensitizer.

8214. The method of item 8080 wherein the agent is a protein kinase C inhibitor.

8215. The method of item 8080 wherein the agent is a ROCK (rho-associated kinase) inhibitor.

8216. The method of item 8080 wherein the agent is a CXCR3 inhibitor.

8217. The method of item 8080 wherein the agent is an Itk inhibitor.

8218. The method of item 8080 wherein the agent is a cytosolic phospholipase A₂-alpha inhibitor.

8219. The method of item 8080 wherein the agent is a PPAR agonist.

8220. The method of item 8080 wherein the agent is an immunosuppressant.

8221. The method of item 8080 wherein the agent is an Erb inhibitor.

8222. The method of item 8080 wherein the agent is an apoptosis agonist.

8223. The method of item 8080 wherein the agent is a lipocortin agonist.

8224. The method of item 8080 wherein the agent is a VCAM-1 antagonist.

8225. The method of item 8080 wherein the agent is a collagen antagonist.

8226. The method of item 8080 wherein the agent is an alpha 2 integrin antagonist.

8227. The method of item 8080 wherein the agent is a TNF alpha inhibitor.

8228. The method of item 8080 wherein the agent is a nitric oxide inhibitor.

8229. The method of item 8080 wherein the agent is a cathepsin inhibitor.

8230. The method of item 8080 wherein the agent is not an anti-inflammatory agent.

8231. The method of item 8080 wherein the agent is not a steroid.

8232. The method of item 8080 wherein the agent is not a glucocorticosteroid.

8233. The method of item 8080 wherein the agent is not dexamethasone.

8234. The method of item 8080 wherein the agent is not an anti-infective agent.

8235. The method of item 8080 wherein the agent is not an antibiotic.

8236. The method of item 8080 wherein the agent is not an anti-fungal agent.

8237. The method of item 8080, wherein the composition comprises a polymer.

8238. The method of item 8080, wherein the composition comprises a polymer, and the polymer is, or comprises, a copolymer.

8239. The method of item 8080, wherein the composition comprises a polymer, and the polymer is, or comprises, a block copolymer.

8240. The method of item 8080, wherein the composition comprises a polymer, and the polymer is, or comprises, a random copolymer.

8241. The method of item 8080, wherein the composition comprises a polymer, and the polymer is, or comprises, a biodegradable polymer.

8242. The method of item 8080, wherein the composition comprises a polymer, and the polymer is, or comprises, a non-biodegradable polymer.

8243. The method of item 8080, wherein the composition comprises a polymer, and the polymer is, or comprises, a hydrophilic polymer.

8244. The method of item 8080, wherein the composition comprises a polymer, and the polymer is, or comprises, a hydrophobic polymer.

8245. The method of item 8080, wherein the composition comprises a polymer, and the polymer is, or comprises, a polymer having hydrophilic domains.

8246. The method of item 8080, wherein the composition comprises a polymer, and the polymer is, or comprises, a polymer having hydrophobic domains.

8247. The method of item 8080, wherein the composition comprises a polymer, and the polymer is, or comprises, a non-conductive polymer.

8248. The method of item 8080, wherein the composition comprises a polymer, and the polymer is, or comprises, an elastomer.

8249. The method of item 8080, wherein the composition comprises a polymer, and the polymer is, or comprises, a hydrogel.

8250. The method of item 8080, wherein the composition comprises a polymer, and the polymer is, or comprises, a silicone polymer.

8251. The method of item 8080, wherein the composition comprises a polymer, and the polymer is, or comprises, a hydrocarbon polymer.

8252. The method of item 8080, wherein the composition comprises a polymer, and the polymer is, or comprises, a styrene-derived polymer.

8253. The method of item 8080, wherein the composition comprises a polymer, and the polymer is, or comprises, a butadiene-derived polymer.

8254. The method of item 8080, wherein the composition comprises a polymer, and the polymer is, or comprises, a macromer.

8255. The method of item 8080, wherein the composition comprises a polymer, and the polymer is, or comprises, a poly(ethylene glycol)polymer.

8256. The method of item 8080, wherein the composition comprises a polymer, and the polymer is, or comprises, an amorphous polymer.

8257. The method of item 8080, wherein the composition further comprises a second pharmaceutically active agent.

8258. The method of item 8080, wherein the composition further comprises an anti-inflammatory agent.

8259. The method of item 8080, wherein the composition further comprises an agent that inhibits infection.

8260. The method of item 8080, wherein the composition further comprises an anthracycline.

8261. The method of item 8080, wherein the composition further comprises doxorubicin.

8262. The method of item 8080 wherein the composition further comprises mitoxantrone.

8263. The method of item 8080 wherein the composition further comprises a fluoropyrimidine.

8264. The method of item 8080, wherein the composition further comprises 5-fluorouracil (5-FU).

8265. The method of item 8080, wherein the composition further comprises a folic acid antagonist.

8266. The method of item 8080, wherein the composition further comprises methotrexate.

8267. The method of item 8080, wherein the composition further comprises a podophylotoxin.

8268. The method of item 8080, wherein the composition further comprises etoposide.

8269. The method of item 8080, wherein the composition further comprises camptothecin.

8270. The method of item 8080, wherein the composition further comprises a hydroxyurea.

8271. The method of item 8080, wherein the composition further comprises a platinum complex.

8272. The method of item 8080, wherein the composition further comprises cisplatin.

8273. The method of item 8080 wherein the composition further comprises an anti-thrombotic agent.

8274. The method of item 8080, wherein the composition further comprises a visualization agent.

8275. The method of item 8080, wherein the composition further comprises a visualization agent, and the visualization agent is a radiopaque material, wherein the radiopaque material comprises a metal, a halogenated compound, or a barium containing compound.

8276. The method of item 8080, wherein the composition further comprises a visualization agent, and the visualization agent is, or comprises, barium, tantalum, or technetium.

8277. The method of item 8080, wherein the composition further comprises a visualization agent, and the visualization agent is, or comprises, an MRI responsive material.

8278. The method of item 8080, wherein the composition further comprises a visualization agent, and the visualization agent is, or comprises, a gadolinium chelate.

8279. The method of item 8080, wherein the composition further comprises a visualization agent, and the visualization agent is, or comprises, iron, magnesium, manganese, copper, or chromium.

8280. The method of item 8080, wherein the composition further comprises a visualization agent, and the visualization agent is, or comprises, iron oxide compound.

8281. The method of item 8080, wherein the composition further comprises a visualization agent, and the visualization agent is, or comprises, a dye, pigment, or colorant.

8282. The method of item 8080 wherein the agent is released in effective concentrations from the composition comprising the agent by diffusion over a period ranging from the time of administration to about 90 days.

8283. The method of item 8080 wherein the agent is released in effective concentrations from the composition comprising the agent by erosion of the composition over a period ranging from the time of administration to about 90 days.

8284. The method of item 8080 wherein the composition further comprises an inflammatory cytokine.

8285. The method of item 8080 wherein the composition further comprises an agent that stimulates cell proliferation.

8286. The method of item 8080 wherein the composition further comprises a polymeric carrier.

8287. The method of item 8080 wherein the composition is in the form of a gel, paste, or spray.

8288. The method of item 8080 wherein the implant is partially constructed with the agent or the composition.

8289. The method of item 8080 wherein the implant is fully constructed with the agent or the composition.

8290. The method of item 8080 wherein the implant is impregnated with the agent or the composition.

8291. The method of item 8080, wherein the agent or the composition forms a coating, and the coating directly contacts the implant.

8292. The method of item 8080, wherein the agent or the composition forms a coating, and the coating indirectly contacts the implant.

8293. The method of item 8080 wherein the agent or the composition forms a coating, and the coating partially covers the implant.

8294. The method of item 8080, wherein the agent or the composition forms a coating, and the coating completely covers the implant.

8295. The method of item 8080 wherein the agent or the composition is located within pores or holes of the implant.

8296. The method of item 8080 wherein the agent or the composition is located within a channel, lumen, or divet of the implant.

8297. The method of item 8080 wherein the implant further comprising an echogenic material.

8298. The method of item 8080 wherein the implant further comprises an echogenic material, wherein the echogenic material is in the form of a coating.

8299. The method of item 8080 wherein the implant is sterile.

8300. The method of item 8080 wherein the agent is delivered from the implant, wherein the agent is released into tissue in the vicinity of the implant after deployment of the implant.

8301. The method of item 8080 wherein the agent is delivered from the implant, wherein the agent is released into tissue in the vicinity of the implant after deployment of the implant, wherein the tissue is connective tissue.

8302. The method of item 8080 wherein the agent is delivered from the implant, wherein the agent is released into tissue in the vicinity of the implant after deployment of the implant, wherein the tissue is muscle tissue.

8303. The method of item 8080 wherein the agent is delivered from the implant, wherein the agent is released into tissue in the vicinity of the implant after deployment of the implant, wherein the tissue is nerve tissue.

8304. The method of item 8080 wherein the agent is delivered from the implant, wherein the agent is released into tissue in the vicinity of the implant after deployment of the implant, wherein the tissue is epithelium tissue.

8305. The method of item 8080 wherein the agent is delivered from the implant, wherein the agent is released in effective concentrations from the implant over a period ranging from the time of deployment of the implant to about 1 year.

8306. The method of item 8080 wherein the agent is delivered from the implant, wherein the agent is released in effective concentrations from the implant over a period ranging from about 1 month to 6 months.

8307. The method of item 8080 wherein the agent is delivered from the implant, wherein the agent is released in effective concentrations from the implant over a period ranging from about 1-90 days.

8308. The method of item 8080 wherein the agent is delivered from the implant, wherein the agent is released in effective concentrations from the implant at a constant rate.

8309. The method of item 8080 wherein the agent is delivered from the implant, wherein the agent is released in effective concentrations from the implant at an increasing rate.

8310. The method of item 8080 wherein the agent is delivered from the implant, wherein the agent is released in effective concentrations from the implant at a decreasing rate.

8311. The method of item 8080 wherein the agent is delivered from the implant, wherein the implant comprises about 0.01 μg to about 10 μg of the agent.

8312. The method of item 8080 wherein the agent is delivered from the implant, wherein the implant comprises about 10 μg to about 10 mg of the agent.

8313. The method of item 8080 wherein the agent is delivered from the implant, wherein the implant comprises about 10 mg to about 250 mg of the agent.

8314. The method of item 8080 wherein the agent is delivered from the implant, wherein the implant comprises about 250 mg to about 1000 mg of the agent.

8315. The method of item 8080 wherein the agent is delivered from the implant, wherein the implant comprises about 1000 mg to about 2500 mg of the agent.

8316. The method of item 8080 wherein the agent is delivered from the implant, wherein a surface of the implant comprises less than 0.01 μg of the agent per mm² of implant surface to which the agent is applied.

8317. The method of item 8080 wherein the agent is delivered from the implant, wherein a surface of the implant comprises about 0.01 μg to about 1 μg of the agent per mm² of implant surface to which the agent is applied.

8318. The method of item 8080 wherein the agent is delivered from the implant, wherein a surface of the implant comprises about 1 μg to about 10 μg of the agent per mm² of implant surface to which the agent is applied.

8319. The method of item 8080 wherein the agent is delivered from the implant, wherein a surface of the implant comprises about 10 μg to about 250 μg of the agent per mm² of implant surface to which the agent is applied.

8320. The method of item 8080 wherein the agent is delivered from the implant, wherein a surface of the implant comprises about 250 μg to about 1000 μg of the agent per mm² of implant surface to which the agent is applied.

8321. The method of item 8080 wherein the agent is delivered from the implant, wherein a surface of the implant comprises about 1000 μg to about 2500 μg of the agent per mm² of implant surface to which the agent is applied.

8322. The method of item 8080, wherein the implant further comprises a coating, and the coating is a uniform coating.

8323. The method of item 8080, wherein the implant further comprises a coating, and the coating is a non-uniform coating.

8324. The method of item 8080, wherein the implant further comprises a coating, and the coating is a discontinuous coating.

8325. The method of item 8080, wherein the implant further comprises a coating, and the coating is a patterned coating.

8326. The method of item 8080, wherein the implant further comprises a coating, and the coating has a thickness of 100 μm or less.

8327. The method of item 8080, wherein the implant further comprises a coating, and the coating has a thickness of 10 μm or less.

8328. The method of item 8080, wherein the implant further comprises a coating, and the coating adheres to the surface of the implant upon deployment of the implant.

8329. The method of item 8080, wherein the implant further comprises a coating, and the coating is stable at room temperature for a period of at least 1 year.

8330. The method of item 8080, wherein the implant further comprises a coating, and the agent is present in the coating in an amount ranging between about 0.0001% to about 1% by weight.

8331. The method of item 8080, wherein the implant further comprises a coating, and the agent is present in the coating in an amount ranging between about 1% to about 10% by weight.

8332. The method of item 8080, wherein the implant further comprises a coating, and the agent is present in the coating in an amount ranging between about 10% to about 25% by weight.

8333. The method of item 8080, wherein the implant further comprises a coating, and the agent is present in the coating in an amount ranging between about 25% to about 70% by weight.

8334. The method of item 8080, wherein the implant further comprises a coating, and the coating comprises a polymer.

8335. The method of item 8080, wherein the implant comprises a first coating having a first composition and a second coating having a second composition.

8336. The method of item 8080, wherein the implant comprises a first coating having a first composition and a second coating having a second composition, wherein the first composition and the second composition are different.

8337. A method for inhibiting scarring comprising placing a penile implant and an anti-scarring agent or a composition comprising an anti-scarring agent into an animal host, wherein the agent inhibits scarring.

8338. The method of item 8337 wherein the agent inhibits cell regeneration.

8339. The method of item 8337 wherein the agent inhibits angiogenesis.

8340. The method of item 8337 wherein the agent inhibits fibroblast migration.

8341. The method of item 8337 wherein the agent inhibits fibroblast proliferation.

8342. The method of item 8337 wherein the agent inhibits deposition of extracellular matrix.

8343. The method of item 8337 wherein the agent inhibits tissue remodeling.

8344. The method of item 8337 wherein the agent is an angiogenesis inhibitor.

8345. The method of item 8337 wherein the agent is a 5-lipoxygenase inhibitor or antagonist.

8346. The method of item 8337 wherein the agent is a chemokine receptor antagonist.

8347. The method of item 8337 wherein the agent is a cell cycle inhibitor.

8348. The method of item 8337 wherein the agent is a taxane.

8349. The method of item 8337 wherein the agent is an anti-microtubule agent.

8350. The method of item 8337 wherein the agent is paclitaxel.

8351. The method of item 8337 wherein the agent is not paclitaxel.

8352. The method of item 8337 wherein the agent is an analogue or derivative of paclitaxel.

8353. The method of item 8337 wherein the agent is a vinca alkaloid.

8354. The method of item 8337 wherein the agent is camptothecin or an analogue or derivative thereof.

8355. The method of item 8337 wherein the agent is a podophyllotoxin.

8356. The method of item 8337 wherein the agent is a podophyllotoxin, wherein the podophyllotoxin is etoposide or an analogue or derivative thereof.

8357. The method of item 8337 wherein the agent is an anthracycline.

8358. The method of item 8337 wherein the agent is an anthracycline, wherein the anthracycline is doxorubicin or an analogue or derivative thereof.

8359. The method of item 8337 wherein the agent is an anthracycline, wherein the anthracycline is mitoxantrone or an analogue or derivative thereof.

8360. The method of item 8337 wherein the agent is a platinum compound.

8361. The method of item 8337 wherein the agent is a nitrosourea.

8362. The method of item 8337 wherein the agent is a nitroimidazole.

8363. The method of item 8337 wherein the agent is a folic acid antagonist.

8364. The method of item 8337 wherein the agent is a cytidine analogue.

8365. The method of item 8337 wherein the agent is a pyrimidine analogue.

8366. The method of item 8337 wherein the agent is a fluoropyrimidine analogue.

8367. The method of item 8337 wherein the agent is a purine analogue.

8368. The method of item 8337 wherein the agent is a nitrogen mustard or an analogue or derivative thereof.

8369. The method of item 8337 wherein the agent is a hydroxyurea.

8370. The method of item 8337 wherein the agent is a mytomicin or an analogue or derivative thereof.

8371. The method of item 8337 wherein the agent is an alkyl sulfonate.

8372. The method of item 8337 wherein the agent is a benzamide or an analogue or derivative thereof.

8373. The method of item 8337 wherein the agent is a nicotinamide or an analogue or derivative thereof.

8374. The method of item 8337 wherein the agent is a halogenated sugar or an analogue or derivative thereof.

8375. The method of item 8337 wherein the agent is a DNA alkylating agent.

8376. The method of item 8337 wherein the agent is an anti-microtubule agent.

8377. The method of item 8337 wherein the agent is a topoisomerase inhibitor.

8378. The method of item 8337 wherein the agent is a DNA cleaving agent.

8379. The method of item 8337 wherein the agent is an antimetabolite.

8380. The method of item 8337 wherein the agent inhibits adenosine deaminase.

8381. The method of item 8337 wherein the agent inhibits purine ring synthesis.

8382. The method of item 8337 wherein the agent is a nucleotide interconversion inhibitor.

8383. The method of item 8337 wherein the agent inhibits dihydrofolate reduction.

8384. The method of item 8337 wherein the agent blocks thymidine monophosphate.

8385. The method of item 8337 wherein the agent causes DNA damage.

8386. The method of item 8337 wherein the agent is a DNA intercalation agent.

8387. The method of item 8337 wherein the agent is a RNA synthesis inhibitor.

8388. The method of item 8337 wherein the agent is a pyrimidine synthesis inhibitor.

8389. The method of item 8337 wherein the agent inhibits ribonucleotide synthesis or function.

8390. The method of item 8337 wherein the agent inhibits thymidine monophosphate synthesis or function.

8391. The method of item 8337 wherein the agent inhibits DNA synthesis.

8392. The method of item 8337 wherein the agent causes DNA adduct formation.

8393. The method of item 8337 wherein the agent inhibits protein synthesis.

8394. The method of item 8337 wherein the agent inhibits microtubule function.

8395. The method of item 8337 wherein the agent is a cyclin dependent protein kinase inhibitor.

8396. The method of item 8337 wherein the agent is an epidermal growth factor kinase inhibitor.

8397. The method of item 8337 wherein the agent is an elastase inhibitor.

8398. The method of item 8337 wherein the agent is a factor Xa inhibitor.

8399. The method of item 8337 wherein the agent is a farnesyltransferase inhibitor.

8400. The method of item 8337 wherein the agent is a fibrinogen antagonist.

8401. The method of item 8337 wherein the agent is a guanylate cyclase stimulant.

8402. The method of item 8337 wherein the agent is a heat shock protein 90 antagonist.

8403. The method of item 8337 wherein the agent is a heat shock protein 90 antagonist, wherein the heat shock protein 90 antagonist is geldanamycin or an analogue or derivative thereof.

8404. The method of item 8337 wherein the agent is a guanylate cyclase stimulant.

8405. The method of item 8337 wherein the agent is a HMGCoA reductase inhibitor.

8406. The method of item 8337 wherein the agent is a HMGCoA reductase inhibitor, wherein the HMGCoA reductase inhibitor is simvastatin or an analogue or derivative thereof.

8407. The method of item 8337 wherein the agent is a hydroorotate dehydrogenase inhibitor.

8408. The method of item 8337 wherein the agent is an IKK2 inhibitor.

8409. The method of item 8337 wherein the agent is an IL-1 antagonist.

8410. The method of item 8337 wherein the agent is an ICE antagonist.

8411. The method of item 8337 wherein the agent is an IRAK antagonist.

8412. The method of item 8337 wherein the agent is an IL-4 agonist.

8413. The method of item 8337 wherein the agent is an immunomodulatory agent.

8414. The method of item 8337 wherein the agent is sirolimus or an analogue or derivative thereof.

8415. The method of item 8337 wherein the agent is not sirolimus.

8416. The method of item 8337 wherein the agent is everolimus or an analogue or derivative thereof.

8417. The method of item 8337 wherein the agent is tacrolimus or an analogue or derivative thereof.

8418. The method of item 8337 wherein the agent is not tacrolimus.

8419. The method of item 8337 wherein the agent is biolmus or an analogue or derivative thereof.

8420. The method of item 8337 wherein the agent is tresperimus or an analogue or derivative thereof.

8421. The method of item 8337 wherein the agent is auranofin or an analogue or derivative thereof.

8422. The method of item 8337 wherein the agent is 27-0-demethylrapamycin or an analogue or derivative thereof.

8423. The method of item 8337 wherein the agent is gusperimus or an analogue or derivative thereof.

8424. The method of item 8337 wherein the agent is pimecrolimus or an analogue or derivative thereof.

8425. The method of item 8337 wherein the agent is ABT-578 or an analogue or derivative thereof.

8426. The method of item 8337 wherein the agent is an inosine monophosphate dehydrogenase (IMPDH) inhibitor.

8427. The method of item 8337 wherein the agent is an IMPDH inhibitor, wherein the IMPDH inhibitor is mycophenolic acid or an analogue or derivative thereof.

8428. The method of item 8337 wherein the agent is an IMPDH inhibitor, wherein the IMPDH inhibitor is 1-alpha-25 dihydroxy vitamin D₃ or an analogue or derivative thereof.

8429. The method of item 8337 wherein the agent is a leukotriene inhibitor.

8430. The method of item 8337 wherein the agent is a MCP-1 antagonist.

8431. The method of item 8337 wherein the agent is a MMP inhibitor.

8432. The method of item 8337 wherein the agent is an NF kappa B inhibitor.

8433. The method of item 8337 wherein the agent is an NF kappa B inhibitor, wherein the NF kappa B inhibitor is Bay 11-7082.

8434. The method of item 8337 wherein the agent is an NO agonist.

8435. The method of item 8337 wherein the agent is a p38 MAP kinase inhibitor.

8436. The method of item 8337 wherein the agent is a p38 MAP kinase inhibitor, wherein the p38 MAP kinase inhibitor is SB 202190.

8437. The method of item 8337 wherein the agent is a phosphodiesterase inhibitor.

8438. The method of item 8337 wherein the agent is a TGF beta inhibitor.

8439. The method of item 8337 wherein the agent is a thromboxane A2 antagonist.

8440. The method of item 8337 wherein the agent is a TNFa antagonist.

8441. The method of item 8337 wherein the agent is a TACE inhibitor.

8442. The method of item 8337 wherein the agent is a tyrosine kinase inhibitor.

8443. The method of item 8337 wherein the agent is a vitronectin inhibitor.

8444. The method of item 8337 wherein the agent is a fibroblast growth factor inhibitor.

8445. The method of item 8337 wherein the agent is a protein kinase inhibitor.

8446. The method of item 8337 wherein the agent is a PDGF receptor kinase inhibitor.

8447. The method of item 8337 wherein the agent is an endothelial growth factor receptor kinase inhibitor.

8448. The method of item 8337 wherein the agent is a retinoic acid receptor antagonist.

8449. The method of item 8337 wherein the agent is a platelet derived growth factor receptor kinase inhibitor.

8450. The method of item 8337 wherein the agent is a fibronogin antagonist.

8451. The method of item 8337 wherein the agent is an antimycotic agent.

8452. The method of item 8337 wherein the agent is an antimycotic agent, wherein the antimycotic agent is sulconizole.

8453. The method of item 8337 wherein the agent is a bisphosphonate.

8454. The method of item 8337 wherein the agent is a phospholipase A1 inhibitor.

8455. The method of item 8337 wherein the agent is a histamine H1/H2/H3 receptor antagonist.

8456. The method of item 8337 wherein the agent is a macrolide antibiotic.

8457. The method of item 8337 wherein the agent is a GPIIb/IIIa receptor antagonist.

8458. The method of item 8337 wherein the agent is an endothelin receptor antagonist.

8459. The method of item 8337 wherein the agent is a peroxisome proliferator-activated receptor agonist.

8460. The method of item 8337 wherein the agent is an estrogen receptor agent.

8461. The method of item 8337 wherein the agent is a somastostatin analogue.

8462. The method of item 8337 wherein the agent is a neurokinin 1 antagonist.

8463. The method of item 8337 wherein the agent is a neurokinin 3 antagonist.

8464. The method of item 8337 wherein the agent is a VLA-4 antagonist.

8465. The method of item 8337 wherein the agent is an osteoclast inhibitor.

8466. The method of item 8337 wherein the agent is a DNA topoisomerase ATP hydrolyzing inhibitor.

8467. The method of item 8337 wherein the agent is an angiotensin I converting enzyme inhibitor.

8468. The method of item 8337 wherein the agent is an angiotensin II antagonist.

8469. The method of item 8337 wherein the agent is an enkephalinase inhibitor.

8470. The method of item 8337 wherein the agent is a peroxisome proliferator-activated receptor gamma agonist insulin sensitizer.

8471. The method of item 8337 wherein the agent is a protein kinase C inhibitor.

8472. The method of item 8337 wherein the agent is a ROCK (rho-associated kinase) inhibitor.

8473. The method of item 8337 wherein the agent is a CXCR3 inhibitor.

8474. The method of item 8337 wherein the agent is an Itk inhibitor.

8475. The method of item 8337 wherein the agent is a cytosolic phospholipase A₂-alpha inhibitor.

8476. The method of item 8337 wherein the agent is a PPAR agonist.

8477. The method of item 8337 wherein the agent is an immunosuppressant.

8478. The method of item 8337 wherein the agent is an Erb inhibitor.

8479. The method of item 8337 wherein the agent is an apoptosis agonist.

8480. The method of item 8337 wherein the agent is a lipocortin agonist.

8481. The method of item 8337 wherein the agent is a VCAM-1 antagonist.

8482. The method of item 8337 wherein the agent is a collagen antagonist.

8483. The method of item 8337 wherein the agent is an alpha 2 integrin antagonist.

8484. The method of item 8337 wherein the agent is a TNF alpha inhibitor.

8485. The method of item 8337 wherein the agent is a nitric oxide inhibitor.

8486. The method of item 8337 wherein the agent is a cathepsin inhibitor.

8487. The method of item 8337 wherein the agent is not an anti-inflammatory agent.

8488. The method of item 8337 wherein the agent is not a steroid.

8489. The method of item 8337 wherein the agent is not a glucocorticosteroid.

8490. The method of item 8337 wherein the agent is not dexamethasone.

8491. The method of item 8337 wherein the agent is not an anti-infective agent.

8492. The method of item 8337 wherein the agent is not an antibiotic.

8493. The method of item 8337 wherein the agent is not an anti-fungal agent.

8494. The method of item 8337, wherein the composition comprises a polymer.

8495. The method of item 8337, wherein the composition comprises a polymer, and the polymer is, or comprises, a copolymer.

8496. The method of item 8337, wherein the composition comprises a polymer, and the polymer is, or comprises, a block copolymer.

8497. The method of item 8337, wherein the composition comprises a polymer, and the polymer is, or comprises, a random copolymer.

8498. The method of item 8337, wherein the composition comprises a polymer, and the polymer is, or comprises, a biodegradable polymer.

8499. The method of item 8337, wherein the composition comprises a polymer, and the polymer is, or comprises, a non-biodegradable polymer.

8500. The method of item 8337, wherein the composition comprises a polymer, and the polymer is, or comprises, a hydrophilic polymer.

8501. The method of item 8337, wherein the composition comprises a polymer, and the polymer is, or comprises, a hydrophobic polymer.

8502. The method of item 8337, wherein the composition comprises a polymer, and the polymer is, or comprises, a polymer having hydrophilic domains.

8503. The method of item 8337, wherein the composition comprises a polymer, and the polymer is, or comprises, a polymer having hydrophobic domains.

8504. The method of item 8337, wherein the composition comprises a polymer, and the polymer is, or comprises, a non-conductive polymer.

8505. The method of item 8337, wherein the composition comprises a polymer, and the polymer is, or comprises, an elastomer.

8506. The method of item 8337, wherein the composition comprises a polymer, and the polymer is, or comprises, a hydrogel.

8507. The method of item 8337, wherein the composition comprises a polymer, and the polymer is, or comprises, a silicone polymer.

8508. The method of item 8337, wherein the composition comprises a polymer, and the polymer is, or comprises, a hydrocarbon polymer.

8509. The method of item 8337, wherein the composition comprises a polymer, and the polymer is, or comprises, a styrene-derived polymer.

8510. The method of item 8337, wherein the composition comprises a polymer, and the polymer is, or comprises, a butadiene-derived polymer.

8511. The method of item 8337, wherein the composition comprises a polymer, and the polymer is, or comprises, a macromer.

8512. The method of item 8337, wherein the composition comprises a polymer, and the polymer is, or comprises, a poly(ethylene glycol)polymer.

8513. The method of item 8337, wherein the composition comprises a polymer, and the polymer is, or comprises, an amorphous polymer.

8514. The method of item 8337, wherein the composition further comprises a second pharmaceutically active agent.

8515. The method of item 8337, wherein the composition further comprises an anti-inflammatory agent.

8516. The method of item 8337, wherein the composition further comprises an agent that inhibits infection.

8517. The method of item 8337, wherein the composition further comprises an anthracycline.

8518. The method of item 8337, wherein the composition further comprises doxorubicin.

8519. The method of item 8337 wherein the composition further comprises mitoxantrone.

8520. The method of item 8337 wherein the composition further comprises a fluoropyrimidine.

8521. The method of item 8337, wherein the composition further comprises 5-fluorouracil (5-FU).

8522. The method of item 8337, wherein the composition further comprises a folic acid antagonist.

8523. The method of item 8337, wherein the composition further comprises methotrexate.

8524. The method of item 8337, wherein the composition further comprises a podophylotoxin.

8525. The method of item 8337, wherein the composition further comprises etoposide.

8526. The method of item 8337, wherein the composition further comprises camptothecin.

8527. The method of item 8337, wherein the composition further comprises a hydroxyurea.

8528. The method of item 8337, wherein the composition further comprises a platinum complex.

8529. The method of item 8337, wherein the composition further comprises cisplatin.

8530. The method of item 8337 wherein the composition further comprises an anti-thrombotic agent.

8531. The method of item 8337, wherein the composition further comprises a visualization agent.

8532. The method of item 8337, wherein the composition further comprises a visualization agent, and the visualization agent is a radiopaque material, wherein the radiopaque material comprises a metal, a halogenated compound, or a barium containing compound.

8533. The method of item 8337, wherein the composition further comprises a visualization agent, and the visualization agent is, or comprises, barium, tantalum, or technetium.

8534. The method of item 8337, wherein the composition further comprises a visualization agent, and the visualization agent is, or comprises, an MRI responsive material.

8535. The method of item 8337, wherein the composition further comprises a visualization agent, and the visualization agent is, or comprises, a gadolinium chelate.

8536. The method of item 8337, wherein the composition further comprises a visualization agent, and the visualization agent is, or comprises, iron, magnesium, manganese, copper, or chromium.

8537. The method of item 8337, wherein the composition further comprises a visualization agent, and the visualization agent is, or comprises, iron oxide compound.

8538. The method of item 8337, wherein the composition further comprises a visualization agent, and the visualization agent is, or comprises, a dye, pigment, or colorant.

8539. The method of item 8337 wherein the agent is released in effective concentrations from the composition comprising the agent by diffusion over a period ranging from the time of administration to about 90 days.

8540. The method of item 8337 wherein the agent is released in effective concentrations from the composition comprising the agent by erosion of the composition over a period ranging from the time of administration to about 90 days.

8541. The method of item 8337 wherein the composition further comprises an inflammatory cytokine.

8542. The method of item 8337 wherein the composition further comprises an agent that stimulates cell proliferation.

8543. The method of item 8337 wherein the composition further comprises a polymeric carrier.

8544. The method of item 8337 wherein the composition is in the form of a gel, paste, or spray.

8545. The method of item 8337 wherein the implant is partially constructed with the agent or the composition.

8546. The method of item 8337 wherein the implant is fully constructed with the agent or the composition.

8547. The method of item 8337 wherein the implant is impregnated with the agent or the composition.

8548. The method of item 8337, wherein the agent or the composition forms a coating, and the coating directly contacts the implant.

8549. The method of item 8337, wherein the agent or the composition forms a coating, and the coating indirectly contacts the implant.

8550. The method of item 8337 wherein the agent or the composition forms a coating, and the coating partially covers the implant.

8551. The method of item 8337, wherein the agent or the composition forms a coating, and the coating completely covers the implant.

8552. The method of item 8337 wherein the agent or the composition is located within pores or holes of the implant.

8553. The method of item 8337 wherein the agent or the composition is located within a channel, lumen, or divet of the implant.

8554. The method of item 8337 wherein the implant-further comprising an echogenic material.

8555. The method of item 8337 wherein the implant further comprises an echogenic material, wherein the echogenic material is in the form of a coating.

8556. The method of item 8337 wherein the implant is sterile.

8557. The method of item 8337 wherein the agent is delivered from the implant, wherein the agent is released into tissue in the vicinity of the implant after deployment of the implant.

8558. The method of item 8337 wherein the agent is delivered from the implant, wherein the agent is released into tissue in the vicinity of the implant after deployment of the implant, wherein the tissue is connective tissue.

8559. The method of item 8337 wherein the agent is delivered from the implant, wherein the agent is released into tissue in the vicinity of the implant after deployment of the implant, wherein the tissue is muscle tissue.

8560. The method of item 8337 wherein the agent is delivered from the implant, wherein the agent is released into tissue in the vicinity of the implant after deployment of the implant, wherein the tissue is nerve tissue.

8561. The method of item 8337 wherein the agent is delivered from the implant, wherein the agent is released into tissue in the vicinity of the implant after deployment of the implant, wherein the tissue is epithelium tissue.

8562. The method of item 8337 wherein the agent is delivered from the implant, wherein the agent is released in effective concentrations from the implant over a period ranging from the time of deployment of the implant to about 1 year.

8563. The method of item 8337 wherein the agent is delivered from the implant, wherein the agent is released in effective concentrations from the implant over a period ranging from about 1 month to 6 months.

8564. The method of item 8337 wherein the agent is delivered from the implant, wherein the agent is released in effective concentrations from the implant over a period ranging from about 1-90 days.

8565. The method of item 8337 wherein the agent is delivered from the implant, wherein the agent is released in effective concentrations from the implant at a constant rate.

8566. The method of item 8337 wherein the agent is delivered from the implant, wherein the agent is released in effective concentrations from the implant at an increasing rate.

8567. The method of item 8337 wherein the agent is delivered from the implant, wherein the agent is released in effective concentrations from the implant at a decreasing rate.

8568. The method of item 8337 wherein the agent is delivered from the implant, wherein the implant comprises about 0.01 μg to about 10 μg of the agent.

8569. The method of item 8337 wherein the agent is delivered from the implant, wherein the implant comprises about 10 μg to about 10 mg of the agent.

8570. The method of item 8337 wherein the agent is delivered from the implant, wherein the implant comprises about 10 mg to about 250 mg of the agent.

8571. The method of item 8337 wherein the agent is delivered from the implant, wherein the implant comprises about 250 mg to about 1000 mg of the agent.

8572. The method of item 8337 wherein the agent is delivered from the implant, wherein the implant comprises about 1000 mg to about 2500 mg of the agent.

8573. The method of item 8337 wherein the agent is delivered from the implant, wherein a surface of the implant comprises less than 0.01 μg of the agent per mm² of implant surface to which the agent is applied.

8574. The method of item 8337 wherein the agent is delivered from the implant, wherein a surface of the implant comprises about 0.01 μg to about 1 μg of the agent per mm² of implant surface to which the agent is applied.

8575. The method of item 8337 wherein the agent is delivered from the implant, wherein a surface of the implant comprises about 1 μg to about 10 μg of the agent per mm² of implant surface to which the agent is applied.

8576. The method of item 8337 wherein the agent is delivered from the implant, wherein a surface of the implant comprises about 10 μp to about 250 μg of the agent per mm² of implant surface to which the agent is applied.

8577. The method of item 8337 wherein the agent is delivered from the implant, wherein a surface of the implant comprises about 250 μg to about 1000 μg of the agent per mm² of implant surface to which the agent is applied.

8578. The method of item 8337 wherein the agent is delivered from the implant, wherein a surface of the implant comprises about 1000 μg to about 2500 μg of the agent per mm² of implant surface to which the agent is applied.

8579. The method of item 8337, wherein the implant further comprises a coating, and the coating is a uniform coating.

8580. The method of item 8337, wherein the implant further comprises a coating, and the coating is a non-uniform coating.

8581. The method of item 8337, wherein the implant further comprises a coating, and the coating is a discontinuous coating.

8582. The method of item 8337, wherein the implant further comprises a coating, and the coating is a patterned coating.

8583. The method of item 8337, wherein the implant further comprises a coating, and the coating has a thickness of 100 μm or less.

8584. The method of item 8337, wherein the implant further comprises a coating, and the coating has a thickness of 10 μm or less.

8585. The method of item 8337, wherein the implant further comprises a coating, and the coating adheres to the surface of the implant upon deployment of the implant.

8586. The method of item 8337, wherein the implant further comprises a coating, and the coating is stable at room temperature for a period of at least 1 year.

8587. The method of item 8337, wherein the implant further comprises a coating, and the agent is present in the coating in an amount ranging between about 0.0001% to about 1% by weight.

8588. The method of item 8337, wherein the implant further comprises a coating, and the agent is present in the coating in an amount ranging between about 1% to about 10% by weight.

8589. The method of item 8337, wherein the implant further comprises a coating, and the agent is present in the coating in an amount ranging between about 10% to about 25% by weight.

8590. The method of item 8337, wherein the implant further comprises a coating, and the agent is present in the coating in an amount ranging between about 25% to about 70% by weight.

8591. The method of item 8337, wherein the implant further comprises a coating, and the coating comprises a polymer.

8592. The method of item 8337, wherein the implant comprises a first coating having a first composition and a second coating having a second composition.

8593. The method of item 8337, wherein the implant comprises a first coating having a first composition and a second coating having a second composition, wherein the first composition and the second composition are different.

8594. A method for inhibiting scarring comprising placing an endotracheal tube (i.e., an implant) and an anti-scarring agent or a composition comprising an anti-scarring agent into an animal host, wherein the agent inhibits scarring.

8595. The method of item 8594 wherein the agent inhibits cell regeneration.

8596. The method of item 8594 wherein the agent inhibits angiogenesis.

8597. The method of item 8594 wherein the agent inhibits fibroblast migration.

8598. The method of item 8594 wherein the agent inhibits fibroblast proliferation.

8599. The method of item 8594 wherein the agent inhibits deposition of extracellular matrix.

8600. The method of item 8594 wherein the agent inhibits tissue remodeling.

8601. The method of item 8594 wherein the agent is an angiogenesis inhibitor.

8602. The method of item 8594 wherein the agent is a 5-lipoxygenase inhibitor or antagonist.

8603. The method of item 8594 wherein the agent is a chemokine receptor antagonist.

8604. The method of item 8594 wherein the agent is a cell cycle inhibitor.

8605. The method of item 8594 wherein the agent is a taxane.

8606. The method of item 8594 wherein the agent is an anti-microtubule agent.

8607. The method of item 8594 wherein the agent is paclitaxel.

8608. The method of item 8594 wherein the agent is not paclitaxel.

8609. The method of item 8594 wherein the agent is an analogue or derivative of paclitaxel.

8610. The method of item 8594 wherein the agent is a vinca alkaloid.

8611. The method of item 8594 wherein the agent is camptothecin or an analogue or derivative thereof.

8612. The method of item 8594 wherein the agent is a podophyllotoxin.

8613. The method of item 8594 wherein the agent is a podophyllotoxin, wherein the podophyllotoxin is etoposide or an analogue or derivative thereof.

8614. The method of item 8594 wherein the agent is an anthracycline.

8615. The method of item 8594 wherein the agent is an anthracycline, wherein the anthracycline is doxorubicin or an analogue or derivative thereof.

8616. The method of item 8594 wherein the agent is an anthracycline, wherein the anthracycline is mitoxantrone or an analogue or derivative thereof.

8617. The method of item 8594 wherein the agent is a platinum compound.

8618. The method of item 8594 wherein the agent is a nitrosourea.

8619. The method of item 8594 wherein the agent is a nitroimidazole.

8620. The method of item 8594 wherein the agent is a folic acid antagonist.

8621. The method of item 8594 wherein the agent is a cytidine analogue.

8622. The method of item 8594 wherein the agent is a pyrimidine analogue.

8623. The method of item 8594 wherein the agent is a fluoropyrimidine analogue.

8624. The method of item 8594 wherein the agent is a purine analogue.

8625. The method of item 8594 wherein the agent is a nitrogen mustard or an analogue or derivative thereof.

8626. The method of item 8594 wherein the agent is a hydroxyurea.

8627. The method of item 8594 wherein the agent is a mytomicin or an analogue or derivative thereof.

8628. The method of item 8594 wherein the agent is an alkyl sulfonate.

8629. The method of item 8594 wherein the agent is a benzamide or an analogue or derivative thereof.

8630. The method of item 8594 wherein the agent is a nicotinamide or an analogue or derivative thereof.

8631. The method of item 8594 wherein the agent is a halogenated sugar or an analogue or derivative thereof.

8632. The method of item 8594 wherein the agent is a DNA alkylating agent.

8633. The method of item 8594 wherein the agent is an anti-microtubule agent.

8634. The method of item 8594 wherein the agent is a topoisomerase inhibitor.

8635. The method of item 8594 wherein the agent is a DNA cleaving agent.

8636. The method of item 8594 wherein the agent is an antimetabolite.

8637. The method of item 8594 wherein the agent inhibits adenosine deaminase.

8638. The method of item 8594 wherein the agent inhibits purine ring synthesis.

8639. The method of item 8594 wherein the agent is a nucleotide interconversion inhibitor.

8640. The method of item 8594 wherein the agent inhibits dihydrofolate reduction.

8641. The method of item 8594 wherein the agent blocks thymidine monophosphate.

8642. The method of item 8594 wherein the agent causes DNA damage.

8643. The method of item 8594 wherein the agent is a DNA intercalation agent.

8644. The method of item 8594 wherein the agent is a RNA synthesis inhibitor.

8645. The method of item 8594 wherein the agent is a pyrimidine synthesis inhibitor.

8646. The method of item 8594 wherein the agent inhibits ribonucleotide synthesis or function.

8647. The method of item 8594 wherein the agent inhibits thymidine monophosphate synthesis or function.

8648. The method of item 8594 wherein the agent inhibits DNA synthesis.

8649. The method of item 8594 wherein the agent causes DNA adduct formation.

8650. The method of item 8594 wherein the agent inhibits protein synthesis.

8651. The method of item 8594 wherein the agent inhibits microtubule function.

8652. The method of item 8594 wherein the agent is a cyclin dependent protein kinase inhibitor.

8653. The method of item 8594 wherein the agent is an epidermal growth factor kinase inhibitor.

8654. The method of item 8594 wherein the agent is an elastase inhibitor.

8655. The method of item 8594 wherein the agent is a factor Xa inhibitor.

8656. The method of item 8594 wherein the agent is a farnesyltransferase inhibitor.

8657. The method of item 8594 wherein the agent is a fibrinogen antagonist.

8658. The method of item 8594 wherein the agent is a guanylate cyclase stimulant.

8659. The method of item 8594 wherein the agent is a heat shock protein 90 antagonist.

8660. The method of item 8594 wherein the agent is a heat shock protein 90 antagonist, wherein the heat shock protein 90 antagonist is geldanamycin or an analogue or derivative thereof.

8661. The method of item 8594 wherein the agent is a guanylate cyclase stimulant.

8662. The method of item 8594 wherein the agent is a HMGCoA reductase inhibitor.

8663. The method of item 8594 wherein the agent is a HMGCoA reductase inhibitor, wherein the HMGCoA reductase inhibitor is simvastatin or an analogue or derivative thereof.

8664. The method of item 8594 wherein the agent is a hydroorotate dehydrogenase inhibitor.

8665. The method of item 8594 wherein the agent is an IKK2 inhibitor.

8666. The method of item 8594 wherein the agent is an IL-1 antagonist.

8667. The method of item 8594 wherein the agent is an ICE antagonist.

8668. The method of item 8594 wherein the agent is an IRAK antagonist.

8669. The method of item 8594 wherein the agent is an IL-4 agonist.

8670. The method of item 8594 wherein the agent is an immunomodulatory agent.

8671. The method of item 8594 wherein the agent is sirolimus or an analogue or derivative thereof.

8672. The method of item 8594 wherein the agent is not sirolimus.

8673. The method of item 8594 wherein the agent is everolimus or an analogue or derivative thereof.

8674. The method of item 8594 wherein the agent is tacrolimus or an analogue or derivative thereof.

8675. The method of item 8594 wherein the agent is not tacrolimus.

8676. The method of item 8594 wherein the agent is biolmus or an analogue or derivative thereof.

8677. The method of item 8594 wherein the agent is tresperimus or an analogue or derivative thereof.

8678. The method of item 8594 wherein the agent is auranofin or an analogue or derivative thereof.

8679. The method of item 8594 wherein the agent is 27-0-demethylrapamycin or an analogue or derivative thereof.

8680. The method of item 8594 wherein the agent is gusperimus or an analogue or derivative thereof.

8681. The method of item 8594 wherein the agent is pimecrolimus or an analogue or derivative thereof.

8682. The method of item 8594 wherein the agent is ABT-578 or an analogue or derivative thereof.

8683. The method of item 8594 wherein the agent is an inosine monophosphate dehydrogenase (IMPDH) inhibitor.

8684. The method of item 8594 wherein the agent is an IMPDH inhibitor, wherein the IMPDH inhibitor is mycophenolic acid or an analogue or derivative thereof.

8685. The method of item 8594 wherein the agent is an IMPDH inhibitor, wherein the IMPDH inhibitor is 1-alpha-25 dihydroxy vitamin D₃ or an analogue or derivative thereof.

8686. The method of item 8594 wherein the agent is a leukotriene inhibitor.

8687. The method of item 8594 wherein the agent is a MCP-1 antagonist.

8688. The method of item 8594 wherein the agent is a MMP inhibitor.

8689. The method of item 8594 wherein the agent is an NF kappa B inhibitor.

8690. The method of item 8594 wherein the agent is an NF kappa B inhibitor, wherein the NF kappa B inhibitor is Bay 11-7082.

8691. The method of item 8594 wherein the agent is an NO agonist.

8692. The method of item 8594 wherein the agent is a p38 MAP kinase inhibitor.

8693. The method of item 8594 wherein the agent is a p38 MAP kinase inhibitor, wherein the p38 MAP kinase inhibitor is SB 202190.

8694. The method of item 8594 wherein the agent is a phosphodiesterase inhibitor.

8695. The method of item 8594 wherein the agent is a TGF beta inhibitor.

8696. The method of item 8594 wherein the agent is a thromboxane A2 antagonist.

8697. The method of item 8594 wherein the agent is a TNFa antagonist.

8698. The method of item 8594 wherein the agent is a TACE inhibitor.

8699. The method of item 8594 wherein the agent is a tyrosine kinase inhibitor.

8700. The method of item 8594 wherein the agent is a vitronectin inhibitor.

8701. The method of item 8594 wherein the agent is a fibroblast growth factor inhibitor.

8702. The method of item 8594 wherein the agent is a protein kinase inhibitor.

8703. The method of item 8594 wherein the agent is a PDGF receptor kinase inhibitor.

8704. The method of item 8594 wherein the agent is an endothelial growth factor receptor kinase inhibitor.

8705. The method of item 8594 wherein the agent is a retinoic acid receptor antagonist.

8706. The method of item 8594 wherein the agent is a platelet derived growth factor receptor kinase inhibitor.

8707. The method of item 8594 wherein the agent is a fibronogin antagonist.

8708. The method of item 8594 wherein the agent is an antimycotic agent.

8709. The method of item 8594 wherein the agent is an antimycotic agent, wherein the antimycotic agent is sulconizole.

8710. The method of item 8594 wherein the agent is a bisphosphonate.

8711. The method of item 8594 wherein the agent is a phospholipase A1 inhibitor.

8712. The method of item 8594 wherein the agent is a histamine H1/H2/H3 receptor antagonist.

8713. The method of item 8594 wherein the agent is a macrolide antibiotic.

8714. The method of item 8594 wherein the agent is a GPIIb/IIIa receptor antagonist.

8715. The method of item 8594 wherein the agent is an endothelin receptor antagonist.

8716. The method of item 8594 wherein the agent is a peroxisome proliferator-activated receptor agonist.

8717. The method of item 8594 wherein the agent is an estrogen receptor agent.

8718. The method of item 8594 wherein the agent is a somastostatin analogue.

8719. The method of item 8594 wherein the agent is a neurokinin 1 antagonist.

8720. The method of item 8594 wherein the agent is a neurokinin 3 antagonist.

8721. The method of item 8594 wherein the agent is a VLA-4 antagonist.

8722. The method of item 8594 wherein the agent is an osteoclast inhibitor.

8723. The method of item 8594 wherein the agent is a DNA topoisomerase ATP hydrolyzing inhibitor.

8724. The method of item 8594 wherein the agent is an angiotensin I converting enzyme inhibitor.

8725. The method of item 8594 wherein the agent is an angiotensin II antagonist.

8726. The method of item 8594 wherein the agent is an enkephalinase inhibitor.

8727. The method of item 8594 wherein the agent is a peroxisome proliferator-activated receptor gamma agonist insulin sensitizer.

8728. The method of item 8594 wherein the agent is a protein kinase C inhibitor.

8729. The method of item 8594 wherein the agent is a ROCK (rho-associated kinase) inhibitor.

8730. The method of item 8594 wherein the agent is a CXCR3 inhibitor.

8731. The method of item 8594 wherein the agent is an Itk inhibitor.

8732. The method of item 8594 wherein the agent is a cytosolic phospholipase A₂-alpha inhibitor.

8733. The method of item 8594 wherein the agent is a PPAR agonist.

8734. The method of item 8594 wherein the agent is an immunosuppressant.

8735. The method of item 8594 wherein the agent is an Erb inhibitor.

8736. The method of item 8594 wherein the agent is an apoptosis agonist.

8737. The method of item 8594 wherein the agent is a lipocortin agonist.

8738. The method of item 8594 wherein the agent is a VCAM-1 antagonist.

8739. The method of item 8594 wherein the agent is a collagen antagonist.

8740. The method of item 8594 wherein the agent is an alpha 2 integrin antagonist.

8741. The method of item 8594 wherein the agent is a TNF alpha inhibitor.

8742. The method of item 8594 wherein the agent is a nitric oxide inhibitor.

8743. The method of item 8594 wherein the agent is a cathepsin inhibitor.

8744. The method of item 8594 wherein the agent is not an anti-inflammatory agent.

8745. The method of item 8594 wherein the agent is not a steroid.

8746. The method of item 8594 wherein the agent is not a glucocorticosteroid.

8747. The method of item 8594 wherein the agent is not dexamethasone.

8748. The method of item 8594 wherein the agent is not an anti-infective agent.

8749. The method of item 8594 wherein the agent is not an antibiotic.

8750. The method of item 8594 wherein the agent is not an anti-fungal agent.

8751. The method of item 8594, wherein the composition comprises a polymer.

8752. The method of item 8594, wherein the composition comprises a polymer, and the polymer is, or comprises, a copolymer.

8753. The method of item 8594, wherein the composition comprises a polymer, and the polymer is, or comprises, a block copolymer.

8754. The method of item 8594, wherein the composition comprises a polymer, and the polymer is, or comprises, a random copolymer.

8755. The method of item 8594, wherein the composition comprises a polymer, and the polymer is, or comprises, a biodegradable polymer.

8756. The method of item 8594, wherein the composition comprises a polymer, and the polymer is, or comprises, a non-biodegradable polymer.

8757. The method of item 8594, wherein the composition comprises a polymer, and the polymer is, or comprises, a hydrophilic polymer.

8758. The method of item 8594, wherein the composition comprises a polymer, and the polymer is, or comprises, a hydrophobic polymer.

8759. The method of item 8594, wherein the composition comprises a polymer, and the polymer is, or comprises, a polymer having hydrophilic domains.

8760. The method of item 8594, wherein the composition comprises a polymer, and the polymer is, or comprises, a polymer having hydrophobic domains.

8761. The method of item 8594, wherein the composition comprises a polymer, and the polymer is, or comprises, a non-conductive polymer.

8762. The method of item 8594, wherein the composition comprises a polymer, and the polymer is, or comprises, an elastomer.

8763. The method of item 8594, wherein the composition comprises a polymer, and the polymer is, or comprises, a hydrogel.

8764. The method of item 8594, wherein the composition comprises a polymer, and the polymer is, or comprises, a silicone polymer.

8765. The method of item 8594, wherein the composition comprises a polymer, and the polymer is, or comprises, a hydrocarbon polymer.

8766. The method of item 8594, wherein the composition comprises a polymer, and the polymer is, or comprises, a styrene-derived polymer.

8767. The method of item 8594, wherein the composition comprises a polymer, and the polymer is, or comprises, a butadiene-derived polymer.

8768. The method of item 8594, wherein the composition comprises a polymer, and the polymer is, or comprises, a macromer.

8769. The method of item 8594, wherein the composition comprises a polymer, and the polymer is, or comprises, a poly(ethylene glycol)polymer.

8770. The method of item 8594, wherein the composition comprises a polymer, and the polymer is, or comprises, an amorphous polymer.

8771. The method of item 8594, wherein the composition further comprises a second pharmaceutically active agent.

8772. The method of item 8594, wherein the composition further comprises an anti-inflammatory agent.

8773. The method of item 8594, wherein the composition further comprises an agent that inhibits infection.

8774. The method of item 8594, wherein the composition further comprises an anthracycline.

8775. The method of item 8594, wherein the composition further comprises doxorubicin.

8776. The method of item 8594 wherein the composition further comprises mitoxantrone.

8777. The method of item 8594 wherein the composition further comprises a fluoropyrimidine.

8778. The method of item 8594, wherein the composition further comprises 5-fluorouracil (5-FU).

8779. The method of item 8594, wherein the composition further comprises a folic acid antagonist.

8780. The method of item 8594, wherein the composition further comprises methotrexate.

8781. The method of item 8594, wherein the composition further comprises a podophylotoxin.

8782. The method of item 8594, wherein the composition further comprises etoposide.

8783. The method of item 8594, wherein the composition further comprises camptothecin.

8784. The method of item 8594, wherein the composition further comprises a hydroxyurea.

8785. The method of item 8594, wherein the composition further comprises a platinum complex.

8786. The method of item 8594, wherein the composition further comprises cisplatin.

8787. The method of item 8594 wherein the composition further comprises an anti-thrombotic agent.

8788. The method of item 8594, wherein the composition further comprises a visualization agent.

8789. The method of item 8594, wherein the composition further comprises a visualization agent, and the visualization agent is a radiopaque material, wherein the radiopaque material comprises a metal, a halogenated compound, or a barium containing compound.

8790. The method of item 8594, wherein the composition further comprises a visualization agent, and the visualization agent is, or comprises, barium, tantalum, or technetium.

8791. The method of item 8594, wherein the composition further comprises a visualization agent, and the visualization agent is, or comprises, an MRI responsive material.

8792. The method of item 8594, wherein the composition further comprises a visualization agent, and the visualization agent is, or comprises, a gadolinium chelate.

8793. The method of item 8594, wherein the composition further comprises a visualization agent, and the visualization agent is, or comprises, iron, magnesium, manganese, copper, or chromium.

8794. The method of item 8594, wherein the composition further comprises a visualization agent, and the visualization agent is, or comprises, iron oxide compound.

8795. The method of item 8594, wherein the composition further comprises a visualization agent, and the visualization agent is, or comprises, a dye, pigment, or colorant.

8796. The method of item 8594 wherein the agent is released in effective concentrations from the composition comprising the agent by diffusion over a period ranging from the time of administration to about 90 days.

8797. The method of item 8594 wherein the agent is released in effective concentrations from the composition comprising the agent by erosion of the composition over a period ranging from the time of administration to about 90 days.

8798. The method of item 8594 wherein the composition further comprises an inflammatory cytokine.

8799. The method of item 8594 wherein the composition further comprises an agent that stimulates cell proliferation.

8800. The method of item 8594 wherein the composition further comprises a polymeric carrier.

8801. The method of item 8594 wherein the composition is in the form of a gel, paste, or spray.

8802. The method of item 8594 wherein the implant is partially constructed with the agent or the composition.

8803. The method of item 8594 wherein the implant is fully constructed with the agent or the composition.

8804. The method of item 8594 wherein the implant is impregnated with the agent or the composition.

8805. The method of item 8594, wherein the agent or the composition forms a coating, and the coating directly contacts the implant.

8806. The method of item 8594, wherein the agent or the composition forms a coating, and the coating indirectly contacts the implant.

8807. The method of item 8594 wherein the agent or the composition forms a coating, and the coating partially covers the implant.

8808. The method of item 8594, wherein the agent or the composition forms a coating, and the coating completely covers the implant.

8809. The method of item 8594 wherein the agent or the composition is located within pores or holes of the implant.

8810. The method of item 8594 wherein the agent or the composition is located within a channel, lumen, or divet of the implant.

8811. The method of item 8594 wherein the implant further comprising an echogenic material.

8812. The method of item 8594 wherein the implant further comprises an echogenic material, wherein the echogenic material is in the form of a coating.

8813. The method of item 8594 wherein the implant is sterile.

8814. The method of item 8594 wherein the agent is delivered from the implant, wherein the agent is released into tissue in the vicinity of the implant after deployment of the implant.

8815. The method of item 8594 wherein the agent is delivered from the implant, wherein the agent is released into tissue in the vicinity of the implant after deployment of the implant, wherein the tissue is connective tissue.

8816. The method of item 8594 wherein the agent is delivered from the implant, wherein the agent is released into tissue in the vicinity of the implant after deployment of the implant, wherein the tissue is muscle tissue.

8817. The method of item 8594 wherein the agent is delivered from the implant, wherein the agent is released into tissue in the vicinity of the implant after deployment of the implant, wherein the tissue is nerve tissue.

8818. The method of item 8594 wherein the agent is delivered from the implant, wherein the agent is released into tissue in the vicinity of the implant after deployment of the implant, wherein the tissue is epithelium tissue.

8819. The method of item 8594 wherein the agent is delivered from the implant, wherein the agent is released in effective concentrations from the implant over a period ranging from the time of deployment of the implant to about 1 year.

8820. The method of item 8594 wherein the agent is delivered from the implant, wherein the agent is released in effective concentrations from the implant over a period ranging from about 1 month to 6 months.

8821. The method of item 8594 wherein the agent is delivered from the implant, wherein the agent is released in effective concentrations from the implant over a period ranging from about 1-90 days.

8822. The method of item 8594 wherein the agent is delivered from the implant, wherein the agent is released in effective concentrations from the implant at a constant rate.

8823. The method of item 8594 wherein the agent is delivered from the implant, wherein the agent is released in effective concentrations from the implant at an increasing rate.

8824. The method of item 8594 wherein the agent is delivered from the implant, wherein the agent is released in effective concentrations from the implant at a decreasing rate.

8825. The method of item 8594 wherein the agent is delivered from the implant, wherein the implant comprises about 0.01 μg to about 10 μg of the agent.

8826. The method of item 8594 wherein the agent is delivered from the implant, wherein the implant comprises about 10 μg to about 10 mg of the agent.

8827. The method of item 8594 wherein the agent is delivered from the implant, wherein the implant comprises about 10 mg to about 250 mg of the agent.

8828. The method of item 8594 wherein the agent is delivered from the implant, wherein the implant comprises about 250 mg to about 1000 mg of the agent.

8829. The method of item 8594 wherein the agent is delivered from the implant, wherein the implant comprises about 1000 mg to about 2500 mg of the agent.

8830. The method of item 8594 wherein the agent is delivered from the implant, wherein a surface of the implant comprises less than 0.01 μg of the agent per mm² of implant surface to which the agent is applied.

8831. The method of item 8594 wherein the agent is delivered from the implant, wherein a surface of the implant comprises about 0.01 μg to about 1 μg of the agent per mm² of implant surface to which the agent is applied.

8832. The method of item 8594 wherein the agent is delivered from the implant, wherein a surface of the implant comprises about 1 μg to about 10 μg of the agent per mm² of implant surface to which the agent is applied.

8833. The method of item 8594 wherein the agent is delivered from the implant, wherein a surface of the implant comprises about 10 μg to about 250 μg of the agent per mm² of implant surface to which the agent is applied.

8834. The method of item 8594 wherein the agent is delivered from the implant, wherein a surface of the implant comprises about 250 μg to about 1000 μg of the agent per mm² of implant surface to which the agent is applied.

8835. The method of item 8594 wherein the agent is delivered from the implant, wherein a surface of the implant comprises about 1000 μg to about 2500 μg of the agent per mm² of implant surface to which the agent is applied.

8836. The method of item 8594, wherein the implant further comprises a coating, and the coating is a uniform coating.

8837. The method of item 8594, wherein the implant further comprises a coating, and the coating is a non-uniform coating.

8838. The method of item 8594, wherein the implant further comprises a coating, and the coating is a discontinuous coating.

8839. The method of item 8594, wherein the implant further comprises a coating, and the coating is a patterned coating.

8840. The method of item 8594, wherein the implant further comprises a coating, and the coating has a thickness of 100 μm or less.

8841. The method of item 8594, wherein the implant further comprises a coating, and the coating has a thickness of 10 μm or less.

8842. The method of item 8594, wherein the implant further comprises a coating, and the coating adheres to the surface of the implant upon deployment of the implant.

8843. The method of item 8594, wherein the implant further comprises a coating, and the coating is stable at room temperature for a period of at least 1 year.

8844. The method of item 8594, wherein the implant further comprises a coating, and the agent is present in the coating in an amount ranging between about 0.0001% to about 1% by weight.

8845. The method of item 8594, wherein the implant further comprises a coating, and the agent is present in the coating in an amount ranging between about 1% to about 10% by weight.

8846. The method of item 8594, wherein the implant further comprises a coating, and the agent is present in the coating in an amount ranging between about 10% to about 25% by weight.

8847. The method of item 8594, wherein the implant further comprises a coating, and the agent is present in the coating in an amount ranging between about 25% to about 70% by weight.

8848. The method of item 8594, wherein the implant further comprises a coating, and the coating comprises a polymer.

8849. The method of item 8594, wherein the implant comprises a first coating having a first composition and a second coating having a second composition.

8850. The method of item 8594, wherein the implant comprises a first coating having a first composition and a second coating having a second composition, wherein the first composition and the second composition are different.

8851. A method for inhibiting scarring comprising placing a tracheostomy tube (i.e., an implant) and an anti-scarring agent or a composition comprising an anti-scarring agent into an animal host, wherein the agent inhibits scarring.

8852. The method of item 8851 wherein the agent inhibits cell regeneration.

8853. The method of item 8851 wherein the agent inhibits angiogenesis.

8854. The method of item 8851 wherein the agent inhibits fibroblast migration.

8855. The method of item 8851 wherein the agent inhibits fibroblast proliferation.

8856. The method of item 8851 wherein the agent inhibits deposition of extracellular matrix.

8857. The method of item 8851 wherein the agent inhibits tissue remodeling.

8858. The method of item 8851 wherein the agent is an angiogenesis inhibitor.

8859. The method of item 8851 wherein the agent is a 5-lipoxygenase inhibitor or antagonist.

8860. The method of item 8851 wherein the agent is a chemokine receptor antagonist.

8861. The method of item 8851 wherein the agent is a cell cycle inhibitor.

8862. The method of item 8851 wherein the agent is a taxane.

8863. The method of item 8851 wherein the agent is an anti-microtubule agent.

8864. The method of item 8851 wherein the agent is paclitaxel.

8865. The method of item 8851 wherein the agent is not paclitaxel.

8866. The method of item 8851 wherein the agent is an analogue or derivative of paclitaxel.

8867. The method of item 8851 wherein the agent is a vinca alkaloid.

8868. The method of item 8851 wherein the agent is camptothecin or an analogue or derivative thereof.

8869. The method of item 8851 wherein the agent is a podophyllotoxin.

8870. The method of item 8851 wherein the agent is a podophyllotoxin, wherein the podophyllotoxin is etoposide or an analogue or derivative thereof.

8871. The method of item 8851 wherein the agent is an anthracycline.

8872. The method of item 8851 wherein the agent is an anthracycline, wherein the anthracycline is doxorubicin or an analogue or derivative thereof.

8873. The method of item 8851 wherein the agent is an anthracycline, wherein the anthracycline is mitoxantrone or an analogue or derivative thereof.

8874. The method of item 8851 wherein the agent is a platinum compound.

8875. The method of item 8851 wherein the agent is a nitrosourea.

8876. The method of item 8851 wherein the agent is a nitroimidazole.

8877. The method of item 8851 wherein the agent is a folic acid antagonist.

8878. The method of item 8851 wherein the agent is a cytidine analogue.

8879. The method of item 8851 wherein the agent is a pyrimidine analogue.

8880. The method of item 8851 wherein the agent is a fluoropyrimidine analogue.

8881. The method of item 8851 wherein the agent is a purine analogue.

8882. The method of item 8851 wherein the agent is a nitrogen mustard or an analogue or derivative thereof.

8883. The method of item 8851 wherein the agent is a hydroxyurea.

8884. The method of item 8851 wherein the agent is a mytomicin or an analogue or derivative thereof.

8885. The method of item 8851 wherein the agent is an alkyl sulfonate.

8886. The method of item 8851 wherein the agent is a benzamide or an analogue or derivative thereof.

8887. The method of item 8851 wherein the agent is a nicotinamide or an analogue or derivative thereof.

8888. The method of item 8851 wherein the agent is a halogenated sugar or an analogue or derivative thereof.

8889. The method of item 8851 wherein the agent is a DNA alkylating agent.

8890. The method of item 8851 wherein the agent is an anti-microtubule agent.

8891. The method of item 8851 wherein the agent is a topoisomerase inhibitor.

8892. The method of item 8851 wherein the agent is a DNA cleaving agent.

8893. The method of item 8851 wherein the agent is an antimetabolite.

8894. The method of item 8851 wherein the agent inhibits adenosine deaminase.

8895. The method of item 8851 wherein the agent inhibits purine ring synthesis.

8896. The method of item 8851 wherein the agent is a nucleotide interconversion inhibitor.

8897. The method of item 8851 wherein the agent inhibits dihydrofolate reduction.

8898. The method of item 8851 wherein the agent blocks thymidine monophosphate.

8899. The method of item 8851 wherein the agent causes DNA damage.

8900. The method of item 8851 wherein the agent is a DNA intercalation agent.

8901. The method of item 8851 wherein the agent is a RNA synthesis inhibitor.

8902. The method of item 8851 wherein the agent is a pyrimidine synthesis inhibitor.

8903. The method of item 8851 wherein the agent inhibits ribonucleotide synthesis or function.

8904. The method of item 8851 wherein the agent inhibits thymidine monophosphate synthesis or function.

8905. The method of item 8851 wherein the agent inhibits DNA synthesis.

8906. The method of item 8851 wherein the agent causes DNA adduct formation.

8907. The method of item 8851 wherein the agent inhibits protein synthesis.

8908. The method of item 8851 wherein the agent inhibits microtubule function.

8909. The method of item 8851 wherein the agent is a cyclin dependent protein kinase inhibitor.

8910. The method of item 8851 wherein the agent is an epidermal growth factor kinase inhibitor.

8911. The method of item 8851 wherein the agent is an elastase inhibitor.

8912. The method of item 8851 wherein the agent is a factor Xa inhibitor.

8913. The method of item 8851 wherein the agent is a farnesyltransferase inhibitor.

8914. The method of item 8851 wherein the agent is a fibrinogen antagonist.

8915. The method of item 8851 wherein the agent is a guanylate cyclase stimulant.

8916. The method of item 8851 wherein the agent is a heat shock protein 90 antagonist.

8917. The method of item 8851 wherein the agent is a heat shock protein 90 antagonist, wherein the heat shock protein 90 antagonist is geldanamycin or an analogue or derivative thereof.

8918. The method of item 8851 wherein the agent is a guanylate cyclase stimulant.

8919. The method of item 8851 wherein the agent is a HMGCoA reductase inhibitor.

8920. The method of item 8851 wherein the agent is a HMGCoA reductase inhibitor, wherein the HMGCoA reductase inhibitor is simvastatin or an analogue or derivative thereof.

8921. The method of item 8851 wherein the agent is a hydroorotate dehydrogenase inhibitor.

8922. The method of item 8851 wherein the agent is an IKK2 inhibitor.

8923. The method of item 8851 wherein the agent is an IL-1 antagonist.

8924. The method of item 8851 wherein the agent is an ICE antagonist.

8925. The method of item 8851 wherein the agent is an IRAK antagonist.

8926. The method of item 8851 wherein the agent is an IL-4 agonist.

8927. The method of item 8851 wherein the agent is an immunomodulatory agent.

8928. The method of item 8851 wherein the agent is sirolimus or an analogue or derivative thereof.

8929. The method of item 8851 wherein the agent is not sirolimus.

8930. The method of item 8851 wherein the agent is everolimus or an analogue or derivative thereof.

8931. The method of item 8851 wherein the agent is tacrolimus or an analogue or derivative thereof.

8932. The method of item 8851 wherein the agent is not tacrolimus.

8933. The method of item 8851 wherein the agent is biolmus or an analogue or derivative thereof.

8934. The method of item 8851 wherein the agent is tresperimus or an analogue or derivative thereof.

8935. The method of item 8851 wherein the agent is auranofin or an analogue or derivative thereof.

8936. The method of item 8851 wherein the agent is 27-0-demethylrapamycin or an analogue or derivative thereof.

8937. The method of item 8851 wherein the agent is gusperimus or an analogue or derivative thereof.

8938. The method of item 8851 wherein the agent is pimecrolimus or an analogue or derivative thereof.

8939. The method of item 8851 wherein the agent is ABT-578 or an analogue or derivative thereof.

8940. The method of item 8851 wherein the agent is an inosine monophosphate dehydrogenase (IMPDH) inhibitor.

8941. The method of item 8851 wherein the agent is an IMPDH inhibitor, wherein the IMPDH inhibitor is mycophenolic acid or an analogue or derivative thereof.

8942. The method of item 8851 wherein the agent is an IMPDH inhibitor, wherein the IMPDH inhibitor is 1-alpha-25 dihydroxy vitamin D₃ or an analogue or derivative thereof.

8943. The method of item 8851 wherein the agent is a leukotriene inhibitor.

8944. The method of item 8851 wherein the agent is a MCP-1 antagonist.

8945. The method of item 8851 wherein the agent is a MMP inhibitor.

8946. The method of item 8851 wherein the agent is an NF kappa B inhibitor.

8947. The method of item 8851 wherein the agent is an NF kappa B inhibitor, wherein the NF kappa B inhibitor is Bay 11-7082.

8948. The method of item 8851 wherein the agent is an NO agonist.

8949. The method of item 8851 wherein the agent is a p38 MAP kinase inhibitor.

8950. The method of item 8851 wherein the agent is a p38 MAP kinase inhibitor, wherein the p38 MAP kinase inhibitor is SB 202190.

8951. The method of item 8851 wherein the agent is a phosphodiesterase inhibitor.

8952. The method of item 8851 wherein the agent is a TGF beta inhibitor.

8953. The method of item 8851 wherein the agent is a thromboxane A2 antagonist.

8954. The method of item 8851 wherein the agent is a TNFa antagonist.

8955. The method of item 8851 wherein the agent is a TACE inhibitor.

8956. The method of item 8851 wherein the agent is a tyrosine kinase inhibitor.

8957. The method of item 8851 wherein the agent is a vitronectin inhibitor.

8958. The method of item 8851 wherein the agent is a fibroblast growth factor inhibitor.

8959. The method of item 8851 wherein the agent is a protein kinase inhibitor.

8960. The method of item 8851 wherein the agent is a PDGF receptor kinase inhibitor.

8961. The method of item 8851 wherein the agent is an endothelial growth factor receptor kinase inhibitor.

8962. The method of item 8851 wherein the agent is a retinoic acid receptor antagonist.

8963. The method of item 8851 wherein the agent is a platelet derived growth factor receptor kinase inhibitor.

8964. The method of item 8851 wherein the agent is a fibronogin antagonist.

8965. The method of item 8851 wherein the agent is an antimycotic agent.

8966. The method of item 8851 wherein the agent is an antimycotic agent, wherein the antimycotic agent is sulconizole.

8967. The method of item 8851 wherein the agent is a bisphosphonate.

8968. The method of item 8851 wherein the agent is a phospholipase A1 inhibitor.

8969. The method of item 8851 wherein the agent is a histamine H1/H2/H3 receptor antagonist.

8970. The method of item 8851 wherein the agent is a macrolide antibiotic.

8971. The method of item 8851 wherein the agent is a GPIIb/IIIa receptor antagonist.

8972. The method of item 8851 wherein the agent is an endothelin receptor antagonist.

8973. The method of item 8851 wherein the agent is a peroxisome proliferator-activated receptor agonist.

8974. The method of item 8851 wherein the agent is an estrogen receptor agent.

8975. The method of item 8851 wherein the agent is a somastostatin analogue.

8976. The method of item 8851 wherein the agent is a neurokinin 1 antagonist.

8977. The method of item 8851 wherein the agent is a neurokinin 3 antagonist.

8978. The method of item 8851 wherein the agent is a VLA-4 antagonist.

8979. The method of item 8851 wherein the agent is an osteoclast inhibitor.

8980. The method of item 8851 wherein the agent is a DNA topoisomerase ATP hydrolyzing inhibitor.

8981. The method of item 8851 wherein the agent is an angiotensin I converting enzyme inhibitor.

8982. The method of item 8851 wherein the agent is an angiotensin II antagonist.

8983. The method of item 8851 wherein the agent is an enkephalinase inhibitor.

8984. The method of item 8851 wherein the agent is a peroxisome proliferator-activated receptor gamma agonist insulin sensitizer.

8985. The method of item 8851 wherein the agent is a protein kinase C inhibitor.

8986. The method of item 8851 wherein the agent is a ROCK (rho-associated kinase) inhibitor.

8987. The method of item 8851 wherein the agent is a CXCR3 inhibitor.

8988. The method of item 8851 wherein the agent is an Itk inhibitor.

8989. The method of item 8851 wherein the agent is a cytosolic phospholipase A₂-alpha inhibitor.

8990. The method of item 8851 wherein the agent is a PPAR agonist.

8991. The method of item 8851 wherein the agent is an immunosuppressant.

8992. The method of item 8851 wherein the agent is an Erb inhibitor.

8993. The method of item 8851 wherein the agent is an apoptosis agonist.

8994. The method of item 8851 wherein the agent is a lipocortin agonist.

8995. The method of item 8851 wherein the agent is a VCAM-1 antagonist.

8996. The method of item 8851 wherein the agent is a collagen antagonist.

8997. The method of item 8851 wherein the agent is an alpha 2 integrin antagonist.

8998. The method of item 8851 wherein the agent is a TNF alpha inhibitor.

8999. The method of item 8851 wherein the agent is a nitric oxide inhibitor.

9000. The method of item 8851 wherein the agent is a cathepsin inhibitor.

9001. The method of item 8851 wherein the agent is not an anti-inflammatory agent.

9002. The method of item 8851 wherein the agent is not a steroid.

9003. The method of item 8851 wherein the agent is not a glucocorticosteroid.

9004. The method of item 8851 wherein the agent is not dexamethasone.

9005. The method of item 8851 wherein the agent is not an anti-infective agent.

9006. The method of item 8851 wherein the agent is not an antibiotic.

9007. The method of item 8851 wherein the agent is not an anti-fungal agent.

9008. The method of item 8851, wherein the composition comprises a polymer.

9009. The method of item 8851, wherein the composition comprises a polymer, and the polymer is, or comprises, a copolymer.

9010. The method of item 8851, wherein the composition comprises a polymer, and the polymer is, or comprises, a block copolymer.

9011. The method of item 8851, wherein the composition comprises a polymer, and the polymer is, or comprises, a random copolymer.

9012. The method of item 8851, wherein the composition comprises a polymer, and the polymer is, or comprises, a biodegradable polymer.

9013. The method of item 8851, wherein the composition comprises a polymer, and the polymer is, or comprises, a non-biodegradable polymer.

9014. The method of item 8851, wherein the composition comprises a polymer, and the polymer is, or comprises, a hydrophilic polymer.

9015. The method of item 8851, wherein the composition comprises a polymer, and the polymer is, or comprises, a hydrophobic polymer.

9016. The method of item 8851, wherein the composition comprises a polymer, and the polymer is, or comprises, a polymer having hydrophilic domains.

9017. The method of item 8851, wherein the composition comprises a polymer, and the polymer is, or comprises, a polymer having hydrophobic domains.

9018. The method of item 8851, wherein the composition comprises a polymer, and the polymer is, or comprises, a non-conductive polymer.

9019. The method of item 8851, wherein the composition comprises a polymer, and the polymer is, or comprises, an elastomer.

9020. The method of item 8851, wherein the composition comprises a polymer, and the polymer is, or comprises, a hydrogel.

9021. The method of item 8851, wherein the composition comprises a polymer, and the polymer is, or comprises, a silicone polymer.

9022. The method of item 8851, wherein the composition comprises a polymer, and the polymer is, or comprises, a hydrocarbon polymer.

9023. The method of item 8851, wherein the composition comprises a polymer, and the polymer is, or comprises, a styrene-derived polymer.

9024. The method of item 8851, wherein the composition comprises a polymer, and the polymer is, or comprises, a butadiene-derived polymer.

9025. The method of item 8851, wherein the composition comprises a polymer, and the polymer is, or comprises, a macromer.

9026. The method of item 8851, wherein the composition comprises a polymer, and the polymer is, or comprises, a poly(ethylene glycol)polymer.

9027. The method of item 8851, wherein the composition comprises a polymer, and the polymer is, or comprises, an amorphous polymer.

9028. The method of item 8851, wherein the composition further comprises a second pharmaceutically active agent.

9029. The method of item 8851, wherein the composition further comprises an anti-inflammatory agent.

9030. The method of item 8851, wherein the composition further comprises an agent that inhibits infection.

9031. The method of item 8851, wherein the composition further comprises an anthracycline.

9032. The method of item 8851, wherein the composition further comprises doxorubicin.

9033. The method of item 8851 wherein the composition further comprises mitoxantrone.

9034. The method of item 8851 wherein the composition further comprises a fluoropyrimidine.

9035. The method of item 8851, wherein the composition further comprises 5-fluorouracil (5-FU).

9036. The method of item 8851, wherein the composition further comprises a folic acid antagonist.

9037. The method of item 8851, wherein the composition further comprises methotrexate.

9038. The method of item 8851, wherein the composition further comprises a podophylotoxin.

9039. The method of item 8851, wherein the composition further comprises etoposide.

9040. The method of item 8851, wherein the composition further comprises camptothecin.

9041. The method of item 8851, wherein the composition further comprises a hydroxyurea.

9042. The method of item 8851, wherein the composition further comprises a platinum complex.

9043. The method of item 8851, wherein the composition further comprises cisplatin.

9044. The method of item 8851 wherein the composition further comprises an anti-thrombotic agent.

9045. The method of item 8851, wherein the composition further comprises a visualization agent.

9046. The method of item 8851, wherein the composition further comprises a visualization agent, and the visualization agent is a radiopaque material, wherein the radiopaque material comprises a metal, a halogenated compound, or a barium containing compound.

9047. The method of item 8851, wherein the composition further comprises a visualization agent, and the visualization agent is, or comprises, barium, tantalum, or technetium.

9048. The method of item 8851, wherein the composition further comprises a visualization agent, and the visualization agent is, or comprises, an MRI responsive material.

9049. The method of item 8851, wherein the composition further comprises a visualization agent, and the visualization agent is, or comprises, a gadolinium chelate.

9050. The method of item 8851, wherein the composition further comprises a visualization agent, and the visualization agent is, or comprises, iron, magnesium, manganese, copper, or chromium.

9051. The method of item 8851, wherein the composition further comprises a visualization agent, and the visualization agent is, or comprises, iron oxide compound.

9052. The method of item 8851, wherein the composition further comprises a visualization agent, and the visualization agent is, or comprises, a dye, pigment, or colorant.

9053. The method of item 8851 wherein the agent is released in effective concentrations from the composition comprising the agent by diffusion over a period ranging from the time of administration to about 90 days.

9054. The method of item 8851 wherein the agent is released in effective concentrations from the composition comprising the agent by erosion of the composition over a period ranging from the time of administration to about 90 days.

9055. The method of item 8851 wherein the composition further comprises an inflammatory cytokine.

9056. The method of item 8851 wherein the composition further comprises an agent that stimulates cell proliferation.

9057. The method of item 8851 wherein the composition further comprises a polymeric carrier.

9058. The method of item 8851 wherein the composition is in the form of a gel, paste, or spray.

9059. The method of item 8851 wherein the implant is partially constructed with the agent or the composition.

9060. The method of item 8851 wherein the implant is fully constructed with the agent or the composition.

9061. The method of item 8851 wherein the implant is impregnated with the agent or the composition.

9062. The method of item 8851, wherein the agent or the composition forms a coating, and the coating directly contacts the implant.

9063. The method of item 8851, wherein the agent or the composition forms a coating, and the coating indirectly contacts the implant.

9064. The method of item 8851 wherein the agent or the composition forms a coating, and the coating partially covers the implant.

9065. The method of item 8851, wherein the agent or the composition forms a coating, and the coating completely covers the implant.

9066. The method of item 8851 wherein the agent or the composition is located within pores or holes of the implant.

9067. The method of item 8851 wherein the agent or the composition is located within a channel, lumen, or divet of the implant.

9068. The method of item 8851 wherein the implant further comprising an echogenic material.

9069. The method of item 8851 wherein the implant further comprises an echogenic material, wherein the echogenic material is in the form of a coating.

9070. The method of item 8851 wherein the implant is sterile.

9071. The method of item 8851 wherein the agent is delivered from the implant, wherein the agent is released into tissue in the vicinity of the implant after deployment of the implant.

9072. The method of item 8851 wherein the agent is delivered from the implant, wherein the agent is released into tissue in the vicinity of the implant after deployment of the implant, wherein the tissue is connective tissue.

9073. The method of item 8851 wherein the agent is delivered from the implant, wherein the agent is released into tissue in the vicinity of the implant after deployment of the implant, wherein the tissue is muscle tissue.

9074. The method of item 8851 wherein the agent is delivered from the implant, wherein the agent is released into tissue in the vicinity of the implant after deployment of the implant, wherein the tissue is nerve tissue.

9075. The method of item 8851 wherein the agent is delivered from the implant, wherein the agent is released into tissue in the vicinity of the implant after deployment of the implant, wherein the tissue is epithelium tissue.

9076. The method of item 8851 wherein the agent is delivered from the implant, wherein the agent is released in effective concentrations from the implant over a period ranging from the time of deployment of the implant to about 1 year.

9077. The method of item 8851 wherein the agent is delivered from the implant, wherein the agent is released in effective concentrations from the implant over a period ranging from about 1 month to 6 months.

9078. The method of item 8851 wherein the agent is delivered from the implant, wherein the agent is released in effective concentrations from the implant over a period ranging from about 1-90 days.

9079. The method of item 8851 wherein the agent is delivered from the implant, wherein the agent is released in effective concentrations from the implant at a constant rate.

9080. The method of item 8851 wherein the agent is delivered from the implant, wherein the agent is released in effective concentrations from the implant at an increasing rate.

9081. The method of item 8851 wherein the agent is delivered from the implant, wherein the agent is released in effective concentrations from the implant at a decreasing rate.

9082. The method of item 8851 wherein the agent is delivered from the implant, wherein the implant comprises about 0.01 μg to about 10 μg of the agent.

9083. The method of item 8851 wherein the agent is delivered from the implant, wherein the implant comprises about 10 μg to about 10 mg of the agent.

9084. The method of item 8851 wherein the agent is delivered from the implant, wherein the implant comprises about 10 mg to about 250 mg of the agent.

9085. The method of item 8851 wherein the agent is delivered from the implant, wherein the implant comprises about 250 mg to about 1000 mg of the agent.

9086. The method of item 8851 wherein the agent is delivered from the implant, wherein the implant comprises about 1000 mg to about 2500 mg of the agent.

9087. The method of item 8851 wherein the agent is delivered from the implant, wherein a surface of the implant comprises less than 0.01 μg of the agent per mm² of implant surface to which the agent is applied.

9088. The method of item 8851 wherein the agent is delivered from the implant, wherein a surface of the implant comprises about 0.01 μg to about 1 μg of the agent per mm² of implant surface to which the agent is applied.

9089. The method of item 8851 wherein the agent is delivered from the implant, wherein a surface of the implant comprises about 1 μg to about 10 μg of the agent per mm² of implant surface to which the agent is applied.

9090. The method of item 8851 wherein the agent is delivered from the implant, wherein a surface of the implant comprises about 10 μg to about 250 μg of the agent per mm² of implant surface to which the agent is applied.

9091. The method of item 8851 wherein the agent is delivered from the implant, wherein a surface of the implant comprises about 250 μg to about 1000 μg of the agent per mm² of implant surface to which the agent is applied.

9092. The method of item 8851 wherein the agent is delivered from the implant, wherein a surface of the implant comprises about 1000 μg to about 2500 μg of the agent per mm² of implant surface to which the agent is applied.

9093. The method of item 8851, wherein the implant further comprises a coating, and the coating is a uniform coating.

9094. The method of item 8851, wherein the implant further comprises a coating, and the coating is a non-uniform coating.

9095. The method of item 8851, wherein the implant further comprises a coating, and the coating is a discontinuous coating.

9096. The method of item 8851, wherein the implant further comprises a coating, and the coating is a patterned coating.

9097. The method of item 8851, wherein the implant further comprises a coating, and the coating has a thickness of 100 μm or less.

9098. The method of item 8851, wherein the implant further comprises a coating, and the coating has a thickness of 10 μm or less.

9099. The method of item 8851, wherein the implant further comprises a coating, and the coating adheres to the surface of the implant upon deployment of the implant.

9100. The method of item 8851, wherein the implant further comprises a coating, and the coating is stable at room temperature for a period of at least 1 year.

9101. The method of item 8851, wherein the implant further comprises a coating, and the agent is present in the coating in an amount ranging between about 0.0001% to about 1% by weight.

9102. The method of item 8851, wherein the implant further comprises a coating, and the agent is present in the coating in an amount ranging between about 1% to about 10% by weight.

9103. The method of item 8851, wherein the implant further comprises a coating, and the agent is present in the coating in an amount ranging between about 10% to about 25% by weight.

9104. The method of item 8851, wherein the implant further comprises a coating, and the agent is present in the coating in an amount ranging between about 25% to about 70% by weight.

9105. The method of item 8851, wherein the implant further comprises a coating, and the coating comprises a polymer.

9106. The method of item 8851, wherein the implant comprises a first coating having a first composition and a second coating having a second composition.

9107. The method of item 8851, wherein the implant comprises a first coating having a first composition and a second coating having a second composition, wherein the first composition and the second composition are different.

9108. A method for inhibiting scarring comprising placing a gastrointestinal device (i.e., an implant) and an anti-scarring agent or a composition comprising an anti-scarring agent into an animal host, wherein the agent inhibits scarring.

9109. The method of item 9108 wherein the agent inhibits cell regeneration.

9110. The method of item 9108 wherein the agent inhibits angiogenesis.

9111. The method of item 9108 wherein the agent inhibits fibroblast migration.

9112. The method of item 9108 wherein the agent inhibits fibroblast proliferation.

9113. The method of item 9108 wherein the agent inhibits deposition of extracellular matrix.

9114. The method of item 9108 wherein the agent inhibits tissue remodeling.

9115. The method of item 9108 wherein the agent is an angiogenesis inhibitor.

9116. The method of item 9108 wherein the agent is a 5-lipoxygenase inhibitor or antagonist.

9117. The method of item 9108 wherein the agent is a chemokine receptor antagonist.

9118. The method of item 9108 wherein the agent is a cell cycle inhibitor.

9119. The method of item 9108 wherein the agent is a taxane.

9120. The method of item 9108 wherein the agent is an anti-microtubule agent.

9121. The method of item 9108 wherein the agent is paclitaxel.

9122. The method of item 9108 wherein the agent is not paclitaxel.

9123. The method of item 9108 wherein the agent is an analogue or derivative of paclitaxel.

9124. The method of item 9108 wherein the agent is a vinca alkaloid.

9125. The method of item 9108 wherein the agent is camptothecin or an analogue or derivative thereof.

9126. The method of item 9108 wherein the agent is a podophyllotoxin.

9127. The method of item 9108 wherein the agent is a podophyllotoxin, wherein the podophyllotoxin is etoposide or an analogue or derivative thereof.

9128. The method of item 9108 wherein the agent is an anthracycline.

9129. The method of item 9108 wherein the agent is an anthracycline, wherein the anthracycline is doxorubicin or an analogue or derivative thereof.

9130. The method of item 9108 wherein the agent is an anthracycline, wherein the anthracycline is mitoxantrone or an analogue or derivative thereof.

9131. The method of item 9108 wherein the agent is a platinum compound.

9132. The method of item 9108 wherein the agent is a nitrosourea.

9133. The method of item 9108 wherein the agent is a nitroimidazole.

9134. The method of item 9108 wherein the agent is a folic acid antagonist.

9135. The method of item 9108 wherein the agent is a cytidine analogue.

9136. The method of item 9108 wherein the agent is a pyrimidine analogue.

9137. The method of item 9108 wherein the agent is a fluoropyrimidine analogue.

9138. The method of item 9108 wherein the agent is a purine analogue.

9139. The method of item 9108 wherein the agent is a nitrogen mustard or an analogue or derivative thereof.

9140. The method of item 9108 wherein the agent is a hydroxyurea.

9141. The method of item 9108 wherein the agent is a mytomicin or an analogue or derivative thereof.

9142. The method of item 9108 wherein the agent is an alkyl sulfonate.

9143. The method of item 9108 wherein the agent is a benzamide or an analogue or derivative thereof.

9144. The method of item 9108 wherein the agent is a nicotinamide or an analogue or derivative thereof.

9145. The method of item 9108 wherein the agent is a halogenated sugar or an analogue or derivative thereof.

9146. The method of item 9108 wherein the agent is a DNA alkylating agent.

9147. The method of item 9108 wherein the agent is an anti-microtubule agent.

9148. The method of item 9108 wherein the agent is a topoisomerase inhibitor.

9149. The method of item 9108 wherein the agent is a DNA cleaving agent.

9150. The method of item 9108 wherein the agent is an antimetabolite.

9151. The method of item 9108 wherein the agent inhibits adenosine deaminase.

9152. The method of item 9108 wherein the agent inhibits purine ring synthesis.

9153. The method of item 9108 wherein the agent is a nucleotide interconversion inhibitor.

9154. The method of item 9108 wherein the agent inhibits dihydrofolate reduction.

9155. The method of item 9108 wherein the agent blocks thymidine mono phosphate.

9156. The method of item 9108 wherein the agent causes DNA damage.

9157. The method of item 9108 wherein the agent is a DNA intercalation agent.

9158. The method of item 9108 wherein the agent is a RNA synthesis inhibitor.

9159. The method of item 9108 wherein the agent is a pyrimidine synthesis inhibitor.

9160. The method of item 9108 wherein the agent inhibits ribonucleotide synthesis or function.

9161. The method of item 9108 wherein the agent inhibits thymidine monophosphate synthesis or function.

9162. The method of item 9108 wherein the agent inhibits DNA synthesis.

9163. The method of item 9108 wherein the agent causes DNA adduct formation.

9164. The method of item 9108 wherein the agent inhibits protein synthesis.

9165. The method of item 9108 wherein the agent inhibits microtubule function.

9166. The method of item 9108 wherein the agent is a cyclin dependent protein kinase inhibitor.

9167. The method of item 9108 wherein the agent is an epidermal growth factor kinase inhibitor.

9168. The method of item 9108 wherein the agent is an elastase inhibitor.

9169. The method of item 9108 wherein the agent is a factor Xa inhibitor.

9170. The method of item 9108 wherein the agent is a farnesyltransferase inhibitor.

9171. The method of item 9108 wherein the agent is a fibrinogen antagonist.

9172. The method of item 9108 wherein the agent is a guanylate cyclase stimulant.

9173. The method of item 9108 wherein the agent is a heat shock protein 90 antagonist.

9174. The method of item 9108 wherein the agent is a heat shock protein 90 antagonist, wherein the heat shock protein 90 antagonist is geldanamycin or an analogue or derivative thereof.

9175. The method of item 9108 wherein the agent is a guanylate cyclase stimulant.

9176. The method of item 9108 wherein the agent is a HMGCoA reductase inhibitor.

9177. The method of item 9108 wherein the agent is a HMGCoA reductase inhibitor, wherein the HMGCoA reductase inhibitor is simvastatin or an analogue or derivative thereof.

9178. The method of item 9108 wherein the agent is a hydroorotate dehydrogenase inhibitor.

9179. The method of item 9108 wherein the agent is an IKK2 inhibitor.

9180. The method of item 9108 wherein the agent is an IL-1 antagonist.

9181. The method of item 9108 wherein the agent is an ICE antagonist.

9182. The method of item 9108 wherein the agent is an IRAK antagonist.

9183. The method of item 9108 wherein the agent is an IL-4 agonist.

9184. The method of item 9108 wherein the agent is an immunomodulatory agent.

9185. The method of item 9108 wherein the agent is sirolimus or an analogue or derivative thereof.

9186. The method of item 9108 wherein the agent is not sirolimus.

9187. The method of item 9108 wherein the agent is everolimus or an analogue or derivative thereof.

9188. The method of item 9108 wherein the agent is tacrolimus or an analogue or derivative thereof.

9189. The method of item 9108 wherein the agent is not tacrolimus.

9190. The method of item 9108 wherein the agent is biolmus or an analogue or derivative thereof.

9191. The method of item 9108 wherein the agent is tresperimus or an analogue or derivative thereof.

9192. The method of item 9108 wherein the agent is auranofin or an analogue or derivative thereof.

9193. The method of item 9108 wherein the agent is 27-0-demethylrapamycin or an analogue or derivative thereof.

9194. The method of item 9108 wherein the agent is gusperimus or an analogue or derivative thereof.

9195. The method of item 9108 wherein the agent is pimecrolimus or an analogue or derivative thereof.

9196. The method of item 9108 wherein the agent is ABT-578 or an analogue or derivative thereof.

9197. The method of item 9108 wherein the agent is an inosine monophosphate dehydrogenase (IMPDH) inhibitor.

9198. The method of item 9108 wherein the agent is an IMPDH inhibitor, wherein the IMPDH inhibitor is mycophenolic acid or an analogue or derivative thereof.

9199. The method of item 9108 wherein the agent is an IMPDH inhibitor, wherein the IMPDH inhibitor is 1-alpha-25 dihydroxy vitamin D₃ or an analogue or derivative thereof.

9200. The method of item 9108 wherein the agent is a leukotriene inhibitor.

9201. The method of item 9108 wherein the agent is a MCP-1 antagonist.

9202. The method of item 9108 wherein the agent is a MMP inhibitor.

9203. The method of item 9108 wherein the agent is an NF kappa B inhibitor.

9204. The method of item 9108 wherein the agent is an NF kappa B inhibitor, wherein the NF kappa B inhibitor is Bay 11-7082.

9205. The method of item 9108 wherein the agent is an NO agonist.

9206. The method of item 9108 wherein the agent is a p38 MAP kinase inhibitor.

9207. The method of item 9108 wherein the agent is a p38 MAP kinase inhibitor, wherein the p38 MAP kinase inhibitor is SB 202190.

9208. The method of item 9108 wherein the agent is a phosphodiesterase inhibitor.

9209. The method of item 9108 wherein the agent is a TGF beta inhibitor.

9210. The method of item 9108 wherein the agent is a thromboxane A2 antagonist.

9211. The method of item 9108 wherein the agent is a TNFa antagonist.

9212. The method of item 9108 wherein the agent is a TACE inhibitor.

9213. The method of item 9108 wherein the agent is a tyrosine kinase inhibitor.

9214. The method of item 9108 wherein the agent is a vitronectin inhibitor.

9215. The method of item 9108 wherein the agent is a fibroblast growth factor inhibitor.

9216. The method of item 9108 wherein the agent is a protein kinase inhibitor.

9217. The method of item 9108 wherein the agent is a PDGF receptor kinase inhibitor.

9218. The method of item 9108 wherein the agent is an endothelial growth factor receptor kinase inhibitor.

9219. The method of item 9108 wherein the agent is a retinoic acid receptor antagonist.

9220. The method of item 9108 wherein the agent is a platelet derived growth factor receptor kinase inhibitor.

9221. The method of item 9108 wherein the agent is a fibronogin antagonist.

9222. The method of item 9108 wherein the agent is an antimycotic agent.

9223. The method of item 9108 wherein the agent is an antimycotic agent, wherein the antimycotic agent is sulconizole.

9224. The method of item 9108 wherein the agent is a bisphosphonate.

9225. The method of item 9108 wherein the agent is a phospholipase A1 inhibitor.

9226. The method of item 9108 wherein the agent is a histamine H1/H2/H3 receptor antagonist.

9227. The method of item 9108 wherein the agent is a macrolide antibiotic.

9228. The method of item 9108 wherein the agent is a GPIIb/IIIa receptor antagonist.

9229. The method of item 9108 wherein the agent is an endothelin receptor antagonist.

9230. The method of item 9108 wherein the agent is a peroxisome proliferator-activated receptor agonist.

9231. The method of item 9108 wherein the agent is an estrogen receptor agent.

9232. The method of item 9108 wherein the agent is a somastostatin analogue.

9233. The method of item 9108 wherein the agent is a neurokinin 1 antagonist.

9234. The method of item 9108 wherein the agent is a neurokinin 3 antagonist.

9235. The method of item 9108 wherein the agent is a VLA-4 antagonist.

9236. The method of item 9108 wherein the agent is an osteoclast inhibitor.

9237. The method of item 9108 wherein the agent is a DNA topoisomerase ATP hydrolyzing inhibitor.

9238. The method of item 9108 wherein the agent is an angiotensin I converting enzyme inhibitor.

9239. The method of item 9108 wherein the agent is an angiotensin II antagonist.

9240. The method of item 9108 wherein the agent is an enkephalinase inhibitor.

9241. The method of item 9108 wherein the agent is a peroxisome proliferator-activated receptor gamma agonist insulin sensitizer.

9242. The method of item 9108 wherein the agent is a protein kinase C inhibitor.

9243. The method of item 9108 wherein the agent is a ROCK (rho-associated kinase) inhibitor.

9244. The method of item 9108 wherein the agent is a CXCR3 inhibitor.

9245. The method of item 9108 wherein the agent is an Itk inhibitor.

9246. The method of item 9108 wherein the agent is a cytosolic phospholipase A₂-alpha inhibitor.

9247. The method of item 9108 wherein the agent is a PPAR agonist.

9248. The method of item 9108 wherein the agent is an immunosuppressant.

9249. The method of item 9108 wherein the agent is an Erb inhibitor.

9250. The method of item 9108 wherein the agent is an apoptosis agonist.

9251. The method of item 9108 wherein the agent is a lipocortin agonist.

9252. The method of item 9108 wherein the agent is a VCAM-1 antagonist.

9253. The method of item 9108 wherein the agent is a collagen antagonist.

9254. The method of item 9108 wherein the agent is an alpha 2 integrin antagonist.

9255. The method of item 9108 wherein the agent is a TNF alpha inhibitor.

9256. The method of item 9108 wherein the agent is a nitric oxide inhibitor.

9257. The method of item 9108 wherein the agent is a cathepsin inhibitor.

9258. The method of item 9108 wherein the agent is not an anti-inflammatory agent.

9259. The method of item 9108 wherein the agent is not a steroid.

9260. The method of item 9108 wherein the agent is not a glucocorticosteroid.

9261. The method of item 9108 wherein the agent is not dexamethasone.

9262. The method of item 9108 wherein the agent is not an anti-infective agent.

9263. The method of item 9108 wherein the agent is not an antibiotic.

9264. The method of item 9108 wherein the agent is not an anti-fungal agent.

9265. The method of item 9108, wherein the composition comprises a polymer.

9266. The method of item 9108, wherein the composition comprises a polymer, and the polymer is, or comprises, a copolymer.

9267. The method of item 9108, wherein the composition comprises a polymer, and the polymer is, or comprises, a block copolymer.

9268. The method of item 9108, wherein the composition comprises a polymer, and the polymer is, or comprises, a random copolymer.

9269. The method of item 9108, wherein the composition comprises a polymer, and the polymer is, or comprises, a biodegradable polymer.

9270. The method of item 9108, wherein the composition comprises a polymer, and the polymer is, or comprises, a non-biodegradable polymer.

9271. The method of item 9108, wherein the composition comprises a polymer, and the polymer is, or comprises, a hydrophilic polymer.

9272. The method of item 9108, wherein the composition comprises a polymer, and the polymer is, or comprises, a hydrophobic polymer.

9273. The method of item 9108, wherein the composition comprises a polymer, and the polymer is, or comprises, a polymer having hydrophilic domains.

9274. The method of item 9108, wherein the composition comprises a polymer, and the polymer is, or comprises, a polymer having hydrophobic domains.

9275. The method of item 9108, wherein the composition comprises a polymer, and the polymer is, or comprises, a non-conductive polymer.

9276. The method of item 9108, wherein the composition comprises a polymer, and the polymer is, or comprises, an elastomer.

9277. The method of item 9108, wherein the composition comprises a polymer, and the polymer is, or comprises, a hydrogel.

9278. The method of item 9108, wherein the composition comprises a polymer, and the polymer is, or comprises, a silicone polymer.

9279. The method of item 9108, wherein the composition comprises a polymer, and the polymer is, or comprises, a hydrocarbon polymer.

9280. The method of item 9108, wherein the composition comprises a polymer, and the polymer is, or comprises, a styrene-derived polymer.

9281. The method of item 9108, wherein the composition comprises a polymer, and the polymer is, or comprises, a butadiene-derived polymer.

9282. The method of item 9108, wherein the composition comprises a polymer, and the polymer is, or comprises, a macromer.

9283. The method of item 9108, wherein the composition comprises a polymer, and the polymer is, or comprises, a poly(ethylene glycol)polymer.

9284. The method of item 9108, wherein the composition comprises a polymer, and the polymer is, or comprises, an amorphous polymer.

9285. The method of item 9108, wherein the composition further comprises a second pharmaceutically active agent.

9286. The method of item 9108, wherein the composition further comprises an anti-inflammatory agent.

9287. The method of item 9108, wherein the composition further comprises an agent that inhibits infection.

9288. The method of item 9108, wherein the composition further comprises an anthracycline.

9289. The method of item 9108, wherein the composition further comprises doxorubicin.

9290. The method of item 9108 wherein the composition further comprises mitoxantrone.

9291. The method of item 9108 wherein the composition further comprises a fluoropyrimidine.

9292. The method of item 9108, wherein the composition further comprises 5-fluorouracil (5-FU).

9293. The method of item 9108, wherein the composition further comprises a folic acid antagonist.

9294. The method of item 9108, wherein the composition further comprises methotrexate.

9295. The method of item 9108, wherein the composition further comprises a podophylotoxin.

9296. The method of item 9108, wherein the composition further comprises etoposide.

9297. The method of item 9108, wherein the composition further comprises camptothecin.

9298. The method of item 9108, wherein the composition further comprises a hydroxyurea.

9299. The method of item 9108, wherein the composition further comprises a platinum complex.

9300. The method of item 9108, wherein the composition further comprises cisplatin.

9301. The method of item 9108 wherein the composition further comprises an anti-thrombotic agent.

9302. The method of item 9108, wherein the composition further comprises a visualization agent.

9303. The method of item 9108, wherein the composition further comprises a visualization agent, and the visualization agent is a radiopaque material, wherein the radiopaque material comprises a metal, a halogenated compound, or a barium containing compound.

9304. The method of item 9108, wherein the composition further comprises a visualization agent, and the visualization agent is, or comprises, barium, tantalum, or technetium.

9305. The method of item 9108, wherein the composition further comprises a visualization agent, and the visualization agent is, or comprises, an MRI responsive material.

9306. The method of item 9108, wherein the composition further comprises a visualization agent, and the visualization agent is, or comprises, a gadolinium chelate.

9307. The method of item 9108, wherein the composition further comprises a visualization agent, and the visualization agent is, or comprises, iron, magnesium, manganese, copper, or chromium.

9308. The method of item 9108, wherein the composition further comprises a visualization agent, and the visualization agent is, or comprises, iron oxide compound.

9309. The method of item 9108, wherein the composition further comprises a visualization agent, and the visualization agent is, or comprises, a dye, pigment, or colorant.

9310. The method of item 9108 wherein the agent is released in effective concentrations from the composition comprising the agent by diffusion over a period ranging from the time of administration to about 90 days.

9311. The method of item 9108 wherein the agent is released in effective concentrations from the composition comprising the agent by erosion of the composition over a period ranging from the time of administration to about 90 days.

9312. The method of item 9108 wherein the composition further comprises an inflammatory cytokine.

9313. The method of item 9108 wherein the composition further comprises an agent that stimulates cell proliferation.

9314. The method of item 9108 wherein the composition further comprises a polymeric carrier.

9315. The method of item 9108 wherein the composition is in the form of a gel, paste, or spray.

9316. The method of item 9108 wherein the implant is partially constructed with the agent or the composition.

9317. The method of item 9108 wherein the implant is fully constructed with the agent or the composition.

9318. The method of item 9108 wherein the implant is impregnated with the agent or the composition.

9319. The method of item 9108, wherein the agent or the composition forms a coating, and the coating directly contacts the implant.

9320. The method of item 9108, wherein the agent or the composition forms a coating, and the coating indirectly contacts the implant.

9321. The method of item 9108 wherein the agent or the composition forms a coating, and the coating partially covers the implant.

9322. The method of item 9108, wherein the agent or the composition forms a coating, and the coating completely covers the implant.

9323. The method of item 9108 wherein the agent or the composition is located within pores or holes of the implant.

9324. The method of item 9108 wherein the agent or the composition is located within a channel, lumen, or divet of the implant.

9325. The method of item 9108 wherein the implant further comprising an echogenic material.

9326. The method of item 9108 wherein the implant further comprises an echogenic material, wherein the echogenic material is in the form of a coating.

9327. The method of item 9108 wherein the implant is sterile.

9328. The method of item 9108 wherein the agent is delivered from the implant, wherein the agent is released into tissue in the vicinity of the implant after deployment of the implant.

9329. The method of item 9108 wherein the agent is delivered from the implant, wherein the agent is released into tissue in the vicinity of the implant after deployment of the implant, wherein the tissue is connective tissue.

9330. The method of item 9108 wherein the agent is delivered from the implant, wherein the agent is released into tissue in the vicinity of the implant after deployment of the implant, wherein the tissue is muscle tissue.

9331. The method of item 9108 wherein the agent is delivered from the implant, wherein the agent is released into tissue in the vicinity of the implant after deployment of the implant, wherein the tissue is nerve tissue.

9332. The method of item 9108 wherein the agent is delivered from the implant, wherein the agent is released into tissue in the vicinity of the implant after deployment of the implant, wherein the tissue is epithelium tissue.

9333. The method of item 9108 wherein the agent is delivered from the implant, wherein the agent is released in effective concentrations from the implant over a period ranging from the time of deployment of the implant to about 1 year.

9334. The method of item 9108 wherein the agent is delivered from the implant, wherein the agent is released in effective concentrations from the implant over a period ranging from about 1 month to 6 months.

9335. The method of item 9108 wherein the agent is delivered from the implant, wherein the agent is released in effective concentrations from the implant over a period ranging from about 1-90 days.

9336. The method of item 9108 wherein the agent is delivered from the implant, wherein the agent is released in effective concentrations from the implant at a constant rate.

9337. The method of item 9108 wherein the agent is delivered from the implant, wherein the agent is released in effective concentrations from the implant at an increasing rate.

9338. The method of item 9108 wherein the agent is delivered from the implant, wherein the agent is released in effective concentrations from the implant at a decreasing rate.

9339. The method of item 9108 wherein the agent is delivered from the implant, wherein the implant comprises about 0.01 μg to about 10 μg of the agent.

9340. The method of item 9108 wherein the agent is delivered from the implant, wherein the implant comprises about 10 μg to about 10 mg of the agent.

9341. The method of item 9108 wherein the agent is delivered from the implant, wherein the implant comprises about 10 mg to about 250 mg of the agent.

9342. The method of item 9108 wherein the agent is delivered from the implant, wherein the implant comprises about 250 mg to about 1000 mg of the agent.

9343. The method of item 9108 wherein the agent is delivered from the implant, wherein the implant comprises about 1000 mg to about 2500 mg of the agent.

9344. The method of item 9108 wherein the agent is delivered from the implant, wherein a surface of the implant comprises less than 0.01 μg of the agent per mm² of implant surface to which the agent is applied.

9345. The method of item 9108 wherein the agent is delivered from the implant, wherein a surface of the implant comprises about 0.01 μg to about 1 μg of the agent per mm² of implant surface to which the agent is applied.

9346. The method of item 9108 wherein the agent is delivered from the implant, wherein a surface of the implant comprises about 1 μg to about 10 μg of the agent per mm² of implant surface to which the agent is applied.

9347. The method of item 9108 wherein the agent is delivered from the implant, wherein a surface of the implant comprises about 10 μg to about 250 μg of the agent per mm² of implant surface to which the agent is applied.

9348. The method of item 9108 wherein the agent is delivered from the implant, wherein a surface of the implant comprises about 250 μg to about 1000 μg of the agent per mm² of implant surface to which the agent is applied.

9349. The method of item 9108 wherein the agent is delivered from the implant, wherein a surface of the implant comprises about 1000 μg to about 2500 μg of the agent per mm² of implant surface to which the agent is applied.

9350. The method of item 9108, wherein the implant further comprises a coating, and the coating is a uniform coating.

9351. The method of item 9108, wherein the implant further comprises a coating, and the coating is a non-uniform coating.

9352. The method of item 9108, wherein the implant further comprises a coating, and the coating is a discontinuous coating.

9353. The method of item 9108, wherein the implant further comprises a coating, and the coating is a patterned coating.

9354. The method of item 9108, wherein the implant further comprises a coating, and the coating has a thickness of 100 μm or less.

9355. The method of item 9108, wherein the implant further comprises a coating, and the coating has a thickness of 10 μm or less.

9356. The method of item 9108, wherein the implant further comprises a coating, and the coating adheres to the surface of the implant upon deployment of the implant.

9357. The method of item 9108, wherein the implant further comprises a coating, and the coating is stable at room temperature for a period of at least 1 year.

9358. The method of item 9108, wherein the implant further comprises a coating, and the agent is present in the coating in an amount ranging between about 0.0001% to about 1% by weight.

9359. The method of item 9108, wherein the implant further comprises a coating, and the agent is present in the coating in an amount ranging between about 1% to about 10% by weight.

9360. The method of item 9108, wherein the implant further comprises a coating, and the agent is present in the coating in an amount ranging between about 10% to about 25% by weight.

9361. The method of item 9108, wherein the implant further comprises a coating, and the agent is present in the coating in an amount ranging between about 25% to about 70% by weight.

9362. The method of item 9108, wherein the implant further comprises a coating, and the coating comprises a polymer.

9363. The method of item 9108, wherein the implant comprises a first coating having a first composition and a second coating having a second composition.

9364. The method of item 9108, wherein the implant comprises a first coating having a first composition and a second coating having a second composition, wherein the first composition and the second composition are different.

9365. A method for inhibiting scarring comprising placing a spinal implant and an anti-scarring agent or a composition comprising an anti-scarring agent into an animal host, wherein the agent inhibits scarring.

9366. The method of item 9365 wherein the agent inhibits cell regeneration.

9367. The method of item 9365 wherein the agent inhibits angiogenesis.

9368. The method of item 9365 wherein the agent inhibits fibroblast migration.

9369. The method of item 9365 wherein the agent inhibits fibroblast proliferation.

9370. The method of item 9365 wherein the agent inhibits deposition of extracellular matrix.

9371. The method of item 9365 wherein the agent inhibits tissue remodeling.

9372. The method of item 9365 wherein the agent is an angiogenesis inhibitor.

9373. The method of item 9365 wherein the agent is a 5-lipoxygenase inhibitor or antagonist.

9374. The method of item 9365 wherein the agent is a chemokine receptor antagonist.

9375. The method of item 9365 wherein the agent is a cell cycle inhibitor.

9376. The method of item 9365 wherein the agent is a taxane.

9377. The method of item 9365 wherein the agent is an anti-microtubule agent.

9378. The method of item 9365 wherein the agent is paclitaxel.

9379. The method of item 9365 wherein the agent is not paclitaxel.

9380. The method of item 9365 wherein the agent is an analogue or derivative of paclitaxel.

9381. The method of item 9365 wherein the agent is a vinca alkaloid.

9382. The method of item 9365 wherein the agent is camptothecin or an analogue or derivative thereof.

9383. The method of item 9365 wherein the agent is a podophyllotoxin.

9384. The method of item 9365 wherein the agent is a podophyllotoxin, wherein the podophyllotoxin is etoposide or an analogue or derivative thereof.

9385. The method of item 9365 wherein the agent is an anthracycline.

9386. The method of item 9365 wherein the agent is an anthracycline, wherein the anthracycline is doxorubicin or an analogue or derivative thereof.

9387. The method of item 9365 wherein the agent is an anthracycline, wherein the anthracycline is mitoxantrone or an analogue or derivative thereof.

9388. The method of item 9365 wherein the agent is a platinum compound.

9389. The method of item 9365 wherein the agent is a nitrosourea.

9390. The method of item 9365 wherein the agent is a nitroimidazole.

9391. The method of item 9365 wherein the agent is a folic acid antagonist.

9392. The method of item 9365 wherein the agent is a cytidine analogue.

9393. The method of item 9365 wherein the agent is a pyrimidine analogue.

9394. The method of item 9365 wherein the agent is a fluoropyrimidine analogue.

9395. The method of item 9365 wherein the agent is a purine analogue.

9396. The method of item 9365 wherein the agent is a nitrogen mustard or an analogue or derivative thereof.

9397. The method of item 9365 wherein the agent is a hydroxyurea.

9398. The method of item 9365 wherein the agent is a mytomicin or an analogue or derivative thereof.

9399. The method of item 9365 wherein the agent is an alkyl sulfonate.

9400. The method of item 9365 wherein the agent is a benzamide or an analogue or derivative thereof.

9401. The method of item 9365 wherein the agent is a nicotinamide or an analogue or derivative thereof.

9402. The method of item 9365 wherein the agent is a halogenated sugar or an analogue or derivative thereof.

9403. The method of item 9365 wherein the agent is a DNA alkylating agent.

9404. The method of item 9365 wherein the agent is an anti-microtubule agent.

9405. The method of item 9365 wherein the agent is a topoisomerase inhibitor.

9406. The method of item 9365 wherein the agent is a DNA cleaving agent.

9407. The method of item 9365 wherein the agent is an antimetabolite.

9408. The method of item 9365 wherein the agent inhibits adenosine deaminase.

9409. The method of item 9365 wherein the agent inhibits purine ring synthesis.

9410. The method of item 9365 wherein the agent is a nucleotide interconversion inhibitor.

9411. The method of item 9365 wherein the agent inhibits dihydrofolate reduction.

9412. The method of item 9365 wherein the agent blocks thymidine monophosphate.

9413. The method of item 9365 wherein the agent causes DNA damage.

9414. The method of item 9365 wherein the agent is a DNA intercalation agent.

9415. The method of item 9365 wherein the agent is a RNA synthesis inhibitor.

9416. The method of item 9365 wherein the agent is a pyrimidine synthesis inhibitor.

9417. The method of item 9365 wherein the agent inhibits ribonucleotide synthesis or function.

9418. The method of item 9365 wherein the agent inhibits thymidine monophosphate synthesis or function.

9419. The method of item 9365 wherein the agent inhibits DNA synthesis.

9420. The method of item 9365 wherein the agent causes DNA adduct formation.

9421. The method of item 9365 wherein the agent inhibits protein synthesis.

9422. The method of item 9365 wherein the agent inhibits microtubule function.

9423. The method of item 9365 wherein the agent is a cyclin dependent protein kinase inhibitor.

9424. The method of item 9365 wherein the agent is an epidermal growth factor kinase inhibitor.

9425. The method of item 9365 wherein the agent is an elastase inhibitor.

9426. The method of item 9365 wherein the agent is a factor Xa inhibitor.

9427. The method of item 9365 wherein the agent is a farnesyltransferase inhibitor.

9428. The method of item 9365 wherein the agent is a fibrinogen antagonist.

9429. The method of item 9365 wherein the agent is a guanylate cyclase stimulant.

9430. The method of item 9365 wherein the agent is a heat shock protein 90 antagonist.

9431. The method of item 9365 wherein the agent is a heat shock protein 90 antagonist, wherein the heat shock protein 90 antagonist is geldanamycin or an analogue or derivative thereof.

9432. The method of item 9365 wherein the agent is a guanylate cyclase stimulant.

9433. The method of item 9365 wherein the agent is a HMGCoA reductase inhibitor.

9434. The method of item 9365 wherein the agent is a HMGCoA reductase inhibitor, wherein the HMGCoA reductase inhibitor is simvastatin or an analogue or derivative thereof.

9435. The method of item 9365 wherein the agent is a hydroorotate dehydrogenase inhibitor.

9436. The method of item 9365 wherein the agent is an IKK2 inhibitor.

9437. The method of item 9365 wherein the agent is an IL-1 antagonist.

9438. The method of item 9365 wherein the agent is an ICE antagonist.

9439. The method of item 9365 wherein the agent is an IRAK antagonist.

9440. The method of item 9365 wherein the agent is an IL-4 agonist.

9441. The method of item 9365 wherein the agent is an immunomodulatory agent.

9442. The method of item 9365 wherein the agent is sirolimus or an analogue or derivative thereof.

9443. The method of item 9365 wherein the agent is not sirolimus.

9444. The method of item 9365 wherein the agent is everolimus or an analogue or derivative thereof.

9445. The method of item 9365 wherein the agent is tacrolimus or an analogue or derivative thereof.

9446. The method of item 9365 wherein the agent is not tacrolimus.

9447. The method of item 9365 wherein the agent is biolmus or an analogue or derivative thereof.

9448. The method of item 9365 wherein the agent is tresperimus or an analogue or derivative thereof.

9449. The method of item 9365 wherein the agent is auranofin or an analogue or derivative thereof.

9450. The method of item 9365 wherein the agent is 27-0-demethylrapamycin or an analogue or derivative thereof.

9451. The method of item 9365 wherein the agent is gusperimus or an analogue or derivative thereof.

9452. The method of item 9365 wherein the agent is pimecrolimus or an analogue or derivative thereof.

9453. The method of item 9365 wherein the agent is ABT-578 or an analogue or derivative thereof.

9454. The method of item 9365 wherein the agent is an inosine monophosphate dehydrogenase (IMPDH) inhibitor.

9455. The method of item 9365 wherein the agent is an IMPDH inhibitor, wherein the IMPDH inhibitor is mycophenolic acid or an analogue or derivative thereof.

9456. The method of item 9365 wherein the agent is an IMPDH inhibitor, wherein the IMPDH inhibitor is 1-alpha-25 dihydroxy vitamin D₃ or an analogue or derivative thereof.

9457. The method of item 9365 wherein the agent is a leukotriene inhibitor.

9458. The method of item 9365 wherein the agent is a MCP-1 antagonist.

9459. The method of item 9365 wherein the agent is a MMP inhibitor.

9460. The method of item 9365 wherein the agent is an NF kappa B inhibitor.

9461. The method of item 9365 wherein the agent is an NF kappa B inhibitor, wherein the NF kappa B inhibitor is Bay 11-7082.

9462. The method of item 9365 wherein the agent is an NO agonist.

9463. The method of item 9365 wherein the agent is a p38 MAP kinase inhibitor.

9464. The method of item 9365 wherein the agent is a p38 MAP kinase inhibitor, wherein the p38 MAP kinase inhibitor is SB 202190.

9465. The method of item 9365 wherein the agent is a phosphodiesterase inhibitor.

9466. The method of item 9365 wherein the agent is a TGF beta inhibitor.

9467. The method of item 9365 wherein the agent is a thromboxane A2 antagonist.

9468. The method of item 9365 wherein the agent is a TNFa antagonist.

9469. The method of item 9365 wherein the agent is a TACE inhibitor.

9470. The method of item 9365 wherein the agent is a tyrosine kinase inhibitor.

9471. The method of item 9365 wherein the agent is a vitronectin inhibitor.

9472. The method of item 9365 wherein the agent is a fibroblast growth factor inhibitor.

9473. The method of item 9365 wherein the agent is a protein kinase inhibitor.

9474. The method of item 9365 wherein the agent is a PDGF receptor kinase inhibitor.

9475. The method of item 9365 wherein the agent is an endothelial growth factor receptor kinase inhibitor.

9476. The method of item 9365 wherein the agent is a retinoic acid receptor antagonist.

9477. The method of item 9365 wherein the agent is a platelet derived growth factor receptor kinase inhibitor.

9478. The method of item 9365 wherein the agent is a fibronogin antagonist.

9479. The method of item 9365 wherein the agent is an antimycotic agent.

9480. The method of item 9365 wherein the agent is an antimycotic agent, wherein the antimycotic agent is sulconizole.

9481. The method of item 9365 wherein the agent is a bisphosphonate.

9482. The method of item 9365 wherein the agent is a phospholipase A1 inhibitor.

9483. The method of item 9365 wherein the agent is a histamine H1/H2/H3 receptor antagonist.

9484. The method of item 9365 wherein the agent is a macrolide antibiotic.

9485. The method of item 9365 wherein the agent is a GPIIb/IIIa receptor antagonist.

9486. The method of item 9365 wherein the agent is an endothelin receptor antagonist.

9487. The method of item 9365 wherein the agent is a peroxisome proliferator-activated receptor agonist.

9488. The method of item 9365 wherein the agent is an estrogen receptor agent.

9489. The method of item 9365 wherein the agent is a somastostatin analogue.

9490. The method of item 9365 wherein the agent is a neurokinin 1 antagonist.

9491. The method of item 9365 wherein the agent is a neurokinin 3 antagonist.

9492. The method of item 9365 wherein the agent is a VLA-4 antagonist.

9493. The method of item 9365 wherein the agent is an osteoclast inhibitor.

9494. The method of item 9365 wherein the agent is a DNA topoisomerase ATP hydrolyzing inhibitor.

9495. The method of item 9365 wherein the agent is an angiotensin I converting enzyme inhibitor.

9496. The method of item 9365 wherein the agent is an angiotensin II antagonist.

9497. The method of item 9365 wherein the agent is an enkephalinase inhibitor.

9498. The method of item 9365 wherein the agent is a peroxisome proliferator-activated receptor gamma agonist insulin sensitizer.

9499. The method of item 9365 wherein the agent is a protein kinase C inhibitor.

9500. The method of item 9365 wherein the agent is a ROCK (rho-associated kinase) inhibitor.

9501. The method of item 9365 wherein the agent is a CXCR3 inhibitor.

9502. The method of item 9365 wherein the agent is an Itk inhibitor.

9503. The method of item 9365 wherein the agent is a cytosolic phospholipase A₂-alpha inhibitor.

9504. The method of item 9365 wherein the agent is a PPAR agonist.

9505. The method of item 9365 wherein the agent is an immunosuppressant.

9506. The method of item 9365 wherein the agent is an Erb inhibitor.

9507. The method of item 9365 wherein the agent is an apoptosis agonist.

9508. The method of item 9365 wherein the agent is a lipocortin agonist.

9509. The method of item 9365 wherein the agent is a VCAM-1 antagonist.

9510. The method of item 9365 wherein the agent is a collagen antagonist.

9511. The method of item 9365 wherein the agent is an alpha 2 integrin antagonist.

9512. The method of item 9365 wherein the agent is a TNF alpha inhibitor.

9513. The method of item 9365 wherein the agent is a nitric oxide inhibitor.

9514. The method of item 9365 wherein the agent is a cathepsin inhibitor.

9515. The method of item 9365 wherein the agent is not an anti-inflammatory agent.

9516. The method of item 9365 wherein the agent is not a steroid.

9517. The method of item 9365 wherein the agent is not a glucocorticosteroid.

9518. The method of item 9365 wherein the agent is not dexamethasone.

9519. The method of item 9365 wherein the agent is not an anti-infective agent.

9520. The method of item 9365 wherein the agent is not an antibiotic.

9521. The method of item 9365 wherein the agent is not an anti-fungal agent.

9522. The method of item 9365, wherein the composition comprises a polymer.

9523. The method of item 9365, wherein the composition comprises a polymer, and the polymer is, or comprises, a copolymer.

9524. The method of item 9365, wherein the composition comprises a polymer, and the polymer is, or comprises, a block copolymer.

9525. The method of item 9365, wherein the composition comprises a polymer, and the polymer is, or comprises, a random copolymer.

9526. The method of item 9365, wherein the composition comprises a polymer, and the polymer is, or comprises, a biodegradable polymer.

9527. The method of item 9365, wherein the composition comprises a polymer, and the polymer is, or comprises, a non-biodegradable polymer.

9528. The method of item 9365, wherein the composition comprises a polymer, and the polymer is, or comprises, a hydrophilic polymer.

9529. The method of item 9365, wherein the composition comprises a polymer, and the polymer is, or comprises, a hydrophobic polymer.

9530. The method of item 9365, wherein the composition comprises a polymer, and the polymer is, or comprises, a polymer having hydrophilic domains.

9531. The method of item 9365, wherein the composition comprises a polymer, and the polymer is, or comprises, a polymer having hydrophobic domains.

9532. The method of item 9365, wherein the composition comprises a polymer, and the polymer is, or comprises, a non-conductive polymer.

9533. The method of item 9365, wherein the composition comprises a polymer, and the polymer is, or comprises, an elastomer.

9534. The method of item 9365, wherein the composition comprises a polymer, and the polymer is, or comprises, a hydrogel.

9535. The method of item 9365, wherein the composition comprises a polymer, and the polymer is, or comprises, a silicone polymer.

9536. The method of item 9365, wherein the composition comprises a polymer, and the polymer is, or comprises, a hydrocarbon polymer.

9537. The method of item 9365, wherein the composition comprises a polymer, and the polymer is, or comprises, a styrene-derived polymer.

9538. The method of item 9365, wherein the composition comprises a polymer, and the polymer is, or comprises, a butadiene-derived polymer.

9539. The method of item 9365, wherein the composition comprises a polymer, and the polymer is, or comprises, a macromer.

9540. The method of item 9365, wherein the composition comprises a polymer, and the polymer is, or comprises, a poly(ethylene glycol)polymer.

9541. The method of item 9365, wherein the composition comprises a polymer, and the polymer is, or comprises, an amorphous polymer.

9542. The method of item 9365, wherein the composition further comprises a second pharmaceutically active agent.

9543. The method of item 9365, wherein the composition further comprises an anti-inflammatory agent.

9544. The method of item 9365, wherein the composition further comprises an agent that inhibits infection.

9545. The method of item 9365, wherein the composition further comprises an anthracycline.

9546. The method of item 9365, wherein the composition further comprises doxorubicin.

9547. The method of item 9365 wherein the composition further comprises mitoxantrone.

9548. The method of item 9365 wherein the composition further comprises a fluoropyrimidine.

9549. The method of item 9365, wherein the composition further comprises 5-fluorouracil (5-FU).

9550. The method of item 9365, wherein the composition further comprises a folic acid antagonist.

9551. The method of item 9365, wherein the composition further comprises methotrexate.

9552. The method of item 9365, wherein the composition further comprises a podophylotoxin.

9553. The method of item 9365, wherein the composition further comprises etoposide.

9554. The method of item 9365, wherein the composition further comprises camptothecin.

9555. The method of item 9365, wherein the composition further comprises a hydroxyurea.

9556. The method of item 9365, wherein the composition further comprises a platinum complex.

9557. The method of item 9365, wherein the composition further comprises cisplatin.

9558. The method of item 9365 wherein the composition further comprises an anti-thrombotic agent.

9559. The method of item 9365, wherein the composition further comprises a visualization agent.

9560. The method of item 9365, wherein the composition further comprises a visualization agent, and the visualization agent is a radiopaque material, wherein the radiopaque material comprises a metal, a halogenated compound, or a barium containing compound.

9561. The method of item 9365, wherein the composition further comprises a visualization agent, and the visualization agent is, or comprises, barium, tantalum, or technetium.

9562. The method of item 9365, wherein the composition further comprises a visualization agent, and the visualization agent is, or comprises, an MRI responsive material.

9563. The method of item 9365, wherein the composition further comprises a visualization agent, and the visualization agent is, or comprises, a gadolinium chelate.

9564. The method of item 9365, wherein the composition further comprises a visualization agent, and the visualization agent is, or comprises, iron, magnesium, manganese, copper, or chromium.

9565. The method of item 9365, wherein the composition further comprises a visualization agent, and the visualization agent is, or comprises, iron oxide compound.

9566. The method of item 9365, wherein the composition further comprises a visualization agent, and the visualization agent is, or comprises, a dye, pigment, or colorant.

9567. The method of item 9365 wherein the agent is released in effective concentrations from the composition comprising the agent by diffusion over a period ranging from the time of administration to about 90 days.

9568. The method of item 9365 wherein the agent is released in effective concentrations from the composition comprising the agent by erosion of the composition over a period ranging from the time of administration to about 90 days.

9569. The method of item 9365 wherein the composition further comprises an inflammatory cytokine.

9570. The method of item 9365 wherein the composition further comprises an agent that stimulates cell proliferation.

9571. The method of item 9365 wherein the composition further comprises a polymeric carrier.

9572. The method of item 9365 wherein the composition is in the form of a gel, paste, or spray.

9573. The method of item 9365 wherein the implant is partially constructed with the agent or the composition.

9574. The method of item 9365 wherein the implant is fully constructed with the agent or the composition.

9575. The method of item 9365 wherein the implant is impregnated with the agent or the composition.

9576. The method of item 9365, wherein the agent or the composition forms a coating, and the coating directly contacts the implant.

9577. The method of item 9365, wherein the agent or the composition forms a coating, and the coating indirectly contacts the implant.

9578. The method of item 9365 wherein the agent or the composition forms a coating, and the coating partially covers the implant.

9579. The method of item 9365, wherein the agent or the composition forms a coating, and the coating completely covers the implant.

9580. The method of item 9365 wherein the agent or the composition is located within pores or holes of the implant.

9581. The method of item 9365 wherein the agent or the composition is located within a channel, lumen, or divet of the implant.

9582. The method of item 9365 wherein the implant further comprising an echogenic material.

9583. The method of item 9365 wherein the implant further comprises an echogenic material, wherein the echogenic material is in the form of a coating.

9584. The method of item 9365 wherein the implant is sterile.

9585. The method of item 9365 wherein the agent is delivered from the implant, wherein the agent is released into tissue in the vicinity of the implant after deployment of the implant.

9586. The method of item 9365 wherein the agent is delivered from the implant, wherein the agent is released into tissue in the vicinity of the implant after deployment of the implant, wherein the tissue is connective tissue.

9587. The method of item 9365 wherein the agent is delivered from the implant, wherein the agent is released into tissue in the vicinity of the implant after deployment of the implant, wherein the tissue is muscle tissue.

9588. The method of item 9365 wherein the agent is delivered from the implant, wherein the agent is released into tissue in the vicinity of the implant after deployment of the implant, wherein the tissue is nerve tissue.

9589. The method of item 9365 wherein the agent is delivered from the implant, wherein the agent is released into tissue in the vicinity of the implant after deployment of the implant, wherein the tissue is epithelium tissue.

9590. The method of item 9365 wherein the agent is delivered from the implant, wherein the agent is released in effective concentrations from the implant over a period ranging from the time of deployment of the implant to about 1 year.

9591. The method of item 9365 wherein the agent is delivered from the implant, wherein the agent is released in effective concentrations from the implant over a period ranging from about 1 month to 6 months.

9592. The method of item 9365 wherein the agent is delivered from the implant, wherein the agent is released in effective concentrations from the implant over a period ranging from about 1-90 days.

9593. The method of item 9365 wherein the agent is delivered from the implant, wherein the agent is released in effective concentrations from the implant at a constant rate.

9594. The method of item 9365 wherein the agent is delivered from the implant, wherein the agent is released in effective concentrations from the implant at an increasing rate.

9595. The method of item 9365 wherein the agent is delivered from the implant, wherein the agent is released in effective concentrations from the implant at a decreasing rate.

9596. The method of item 9365 wherein the agent is delivered from the implant, wherein the implant comprises about 0.01 μg to about 10 μg of the agent.

9597. The method of item 9365 wherein the agent is delivered from the implant, wherein the implant comprises about 10 μg to about 10 mg of the agent.

9598. The method of item 9365 wherein the agent is delivered from the implant, wherein the implant comprises about 10 mg to about 250 mg of the agent.

9599. The method of item 9365 wherein the agent is delivered from the implant, wherein the implant comprises about 250 mg to about 1000 mg of the agent.

9600. The method of item 9365 wherein the agent is delivered from the implant, wherein the implant comprises about 1000 mg to about 2500 mg of the agent.

9601. The method of item 9365 wherein the agent is delivered from the implant, wherein a surface of the implant comprises less than 0.01 μg of the agent per mm² of implant surface to which the agent is applied.

9602. The method of item 9365 wherein the agent is delivered from the implant, wherein a surface of the implant comprises about 0.01 μg to about 1 μg of the agent per mm² of implant surface to which the agent is applied.

9603. The method of item 9365 wherein the agent is delivered from the implant, wherein a surface of the implant comprises about 1 μg to about 10 μg of the agent per mm² of implant surface to which the agent is applied.

9604. The method of item 9365 wherein the agent is delivered from the implant, wherein a surface of the implant comprises about 10 μg to about 250 μg of the agent per mm² of implant surface to which the agent is applied.

9605. The method of item 9365 wherein the agent is delivered from the implant, wherein a surface of the implant comprises about 250 μg to about 1000 μg of the agent per mm² of implant surface to which the agent is applied.

9606. The method of item 9365 wherein the agent is delivered from the implant, wherein a surface of the implant comprises about 1000 μg to about 2500 μg of the agent per mm² of implant surface to which the agent is applied.

9607. The method of item 9365, wherein the implant further comprises a coating, and the coating is a uniform coating.

9608. The method of item 9365, wherein the implant further comprises a coating, and the coating is a non-uniform coating.

9609. The method of item 9365, wherein the implant further comprises a coating, and the coating is a discontinuous coating.

9610. The method of item 9365, wherein the implant further comprises a coating, and the coating is a patterned coating.

9611. The method of item 9365, wherein the implant further comprises a coating, and the coating has a thickness of 100 μm or less.

9612. The method of item 9365, wherein the implant further comprises a coating, and the coating has a thickness of 10 μm or less.

9613. The method of item 9365, wherein the implant further comprises a coating, and the coating adheres to the surface of the implant upon deployment of the implant.

9614. The method of item 9365, wherein the implant further comprises a coating, and the coating is stable at room temperature for a period of at least 1 year.

9615. The method of item 9365, wherein the implant further comprises a coating, and the agent is present in the coating in an amount ranging between about 0.0001% to about 1% by weight.

9616. The method of item 9365, wherein the implant further comprises a coating, and the agent is present in the coating in an amount ranging between about 1% to about 10% by weight.

9617. The method of item 9365, wherein the implant further comprises a coating, and the agent is present in the coating in an amount ranging between about 10% to about 25% by weight.

9618. The method of item 9365, wherein the implant further comprises a coating, and the agent is present in the coating in an amount ranging between about 25% to about 70% by weight.

9619. The method of item 9365, wherein the implant further comprises a coating, and the coating comprises a polymer.

9620. The method of item 9365, wherein the implant comprises a first coating having a first composition and a second coating having a second composition.

9621. The method of item 9365, wherein the implant comprises a first coating having a first composition and a second coating having a second composition, wherein the first composition and the second composition are different.

9622. A method for inhibiting scarring comprising placing a pressure monitoring implant and an anti-scarring agent or a composition comprising an anti-scarring agent into an animal host, wherein the agent inhibits scarring.

9623. The method of item 9622 wherein the agent inhibits cell regeneration.

9624. The method of item 9622 wherein the agent inhibits angiogenesis.

9625. The method of item 9622 wherein the agent inhibits fibroblast migration.

9626. The method of item 9622 wherein the agent inhibits fibroblast proliferation.

9627. The method of item 9622 wherein the agent inhibits deposition of extracellular matrix.

9628. The method of item 9622 wherein the agent inhibits tissue remodeling.

9629. The method of item 9622 wherein the agent is an angiogenesis inhibitor.

9630. The method of item 9622 wherein the agent is a 5-lipoxygenase inhibitor or antagonist.

9631. The method of item 9622 wherein the agent is a chemokine receptor antagonist.

9632. The method of item 9622 wherein the agent is a cell cycle inhibitor.

9633. The method of item 9622 wherein the agent is a taxane.

9634. The method of item 9622 wherein the agent is an anti-microtubule agent.

9635. The method of item 9622 wherein the agent is paclitaxel.

9636. The method of item 9622 wherein the agent is not paclitaxel.

9637. The method of item 9622 wherein the agent is an analogue or derivative of paclitaxel.

9638. The method of item 9622 wherein the agent is a vinca alkaloid.

9639. The method of item 9622 wherein the agent is camptothecin or an analogue or derivative thereof.

9640. The method of item 9622 wherein the agent is a podophyllotoxin.

9641. The method of item 9622 wherein the agent is a podophyllotoxin, wherein the podophyllotoxin is etoposide or an analogue or derivative thereof.

9642. The method of item 9622 wherein the agent is an anthracycline.

9643. The method of item 9622 wherein the agent is an anthracycline, wherein the anthracycline is doxorubicin or an analogue or derivative thereof.

9644. The method of item 9622 wherein the agent is an anthracycline, wherein the anthracycline is mitoxantrone or an analogue or derivative thereof.

9645. The method of item 9622 wherein the agent is a platinum compound.

9646. The method of item 9622 wherein the agent is a nitrosourea.

9647. The method of item 9622 wherein the agent is a nitroimidazole.

9648. The method of item 9622 wherein the agent is a folic acid antagonist.

9649. The method of item 9622 wherein the agent is a cytidine analogue.

9650. The method of item 9622 wherein the agent is a pyrimidine analogue.

9651. The method of item 9622 wherein the agent is a fluoropyrimidine analogue.

9652. The method of item 9622 wherein the agent is a purine analogue.

9653. The method of item 9622 wherein the agent is a nitrogen mustard or an analogue or derivative thereof.

9654. The method of item 9622 wherein the agent is a hydroxyurea.

9655. The method of item 9622 wherein the agent is a mytomicin or an analogue or derivative thereof.

9656. The method of item 9622 wherein the agent is an alkyl sulfonate.

9657. The method of item 9622 wherein the agent is a benzamide or an analogue or derivative thereof.

9658. The method of item 9622 wherein the agent is a nicotinamide or an analogue or derivative thereof.

9659. The method of item 9622 wherein the agent is a halogenated sugar or an analogue or derivative thereof.

9660. The method of item 9622 wherein the agent is a DNA alkylating agent.

9661. The method of item 9622 wherein the agent is an anti- microtubule agent.

9662. The method of item 9622 wherein the agent is a topoisomerase inhibitor.

9663. The method of item 9622 wherein the agent is a DNA cleaving agent.

9664. The method of item 9622 wherein the agent is an antimetabolite.

9665. The method of item 9622 wherein the agent inhibits adenosine deaminase.

9666. The method of item 9622 wherein the agent inhibits purine ring synthesis.

9667. The method of item 9622 wherein the agent is a nucleotide interconversion inhibitor.

9668. The method of item 9622 wherein the agent inhibits dihydrofolate reduction.

9669. The method of item 9622 wherein the agent blocks thymidine monophosphate.

9670. The method of item 9622 wherein the agent causes DNA damage.

9671. The method of item 9622 wherein the agent is a DNA intercalation agent.

9672. The method of item 9622 wherein the agent is a RNA synthesis inhibitor.

9673. The method of item 9622 wherein the agent is a pyrimidine synthesis inhibitor.

9674. The method of item 9622 wherein the agent inhibits ribonucleotide synthesis or function.

9675. The method of item 9622 wherein the agent inhibits thymidine monophosphate synthesis or function.

9676. The method of item 9622 wherein the agent inhibits DNA synthesis.

9677. The method of item 9622 wherein the agent causes DNA adduct formation.

9678. The method of item 9622 wherein the agent inhibits protein synthesis.

9679. The method of item 9622 wherein the agent inhibits microtubule function.

9680. The method of item 9622 wherein the agent is a cyclin dependent protein kinase inhibitor.

9681. The method of item 9622 wherein the agent is an epidermal growth factor kinase inhibitor.

9682. The method of item 9622 wherein the agent is an elastase inhibitor.

9683. The method of item 9622 wherein the agent is a factor Xa inhibitor.

9684. The method of item 9622 wherein the agent is a farnesyltransferase inhibitor.

9685. The method of item 9622 wherein the agent is a fibrinogen antagonist.

9686. The method of item 9622 wherein the agent is a guanylate cyclase stimulant.

9687. The method of item 9622 wherein the agent is a heat shock protein 90 antagonist.

9688. The method of item 9622 wherein the agent is a heat shock protein 90 antagonist, wherein the heat shock protein 90 antagonist is geldanamycin or an analogue or derivative thereof.

9689. The method of item 9622 wherein the agent is a guanylate cyclase stimulant.

9690. The method of item 9622 wherein the agent is a HMGCoA reductase inhibitor.

9691. The method of item 9622 wherein the agent is a HMGCoA reductase inhibitor, wherein the HMGCoA reductase inhibitor is simvastatin or an analogue or derivative thereof.

9692. The method of item 9622 wherein the agent is a hydroorotate dehydrogenase inhibitor.

9693. The method of item 9622 wherein the agent is an IKK2 inhibitor.

9694. The method of item 9622 wherein the agent is an IL-1 antagonist.

9695. The method of item 9622 wherein the agent is an ICE antagonist.

9696. The method of item 9622 wherein the agent is an IRAK antagonist.

9697. The method of item 9622 wherein the agent is an IL-4 agonist.

9698. The method of item 9622 wherein the agent is an immunomodulatory agent.

9699. The method of item 9622 wherein the agent is sirolimus or an analogue or derivative thereof.

9700. The method of item 9622 wherein the agent is not sirolimus.

9701. The method of item 9622 wherein the agent is everolimus or an analogue or derivative thereof.

9702. The method of item 9622 wherein the agent is tacrolimus or an analogue or derivative thereof.

9703. The method of item 9622 wherein the agent is not tacrolimus.

9704. The method of item 9622 wherein the agent is biolmus or an analogue or derivative thereof.

9705. The method of item 9622 wherein the agent is tresperimus or an analogue or derivative thereof.

9706. The method of item 9622 wherein the agent is auranofin or an analogue or derivative thereof.

9707. The method of item 9622 wherein the agent is 27-0-demethylrapamycin or an analogue or derivative thereof.

9708. The method of item 9622 wherein the agent is gusperimus or an analogue or derivative thereof.

9709. The method of item 9622 wherein the agent is pimecrolimus or an analogue or derivative thereof.

9710. The method of item 9622 wherein the agent is ABT-578 or an analogue or derivative thereof.

9711. The method of item 9622 wherein the agent is an inosine monophosphate dehydrogenase (IMPDH) inhibitor.

9712. The method of item 9622 wherein the agent is an IMPDH inhibitor, wherein the IMPDH inhibitor is mycophenolic acid or an analogue or derivative thereof.

9713. The method of item 9622 wherein the agent is an IMPDH inhibitor, wherein the IMPDH inhibitor is 1-alpha-25 dihydroxy vitamin D₃ or an analogue or derivative thereof.

9714. The method of item 9622 wherein the agent is a leukotriene inhibitor.

9715. The method of item 9622 wherein the agent is a MCP-1 antagonist.

9716. The method of item 9622 wherein the agent is a MMP inhibitor.

9717. The method of item 9622 wherein the agent is an NF kappa B inhibitor.

9718. The method of item 9622 wherein the agent is an NF kappa B inhibitor, wherein the NF kappa B inhibitor is Bay 11-7082.

9719. The method of item 9622 wherein the agent is an NO agonist.

9720. The method of item 9622 wherein the agent is a p38 MAP kinase inhibitor.

9721. The method of item 9622 wherein the agent is a p38 MAP kinase inhibitor, wherein the p38 MAP kinase inhibitor is SB 202190.

9722. The method of item 9622 wherein the agent is a phosphodiesterase inhibitor.

9723. The method of item 9622 wherein the agent is a TGF beta inhibitor.

9724. The method of item 9622 wherein the agent is a thromboxane A2 antagonist.

9725. The method of item 9622 wherein the agent is a TNFa antagonist.

9726. The method of item 9622 wherein the agent is a TACE inhibitor.

9727. The method of item 9622 wherein the agent is a tyrosine kinase inhibitor.

9728. The method of item 9622 wherein the agent is a vitronectin inhibitor.

9729. The method of item 9622 wherein the agent is a fibroblast growth factor inhibitor.

9730. The method of item 9622 wherein the agent is a protein kinase inhibitor.

9731. The method of item 9622 wherein the agent is a PDGF receptor kinase inhibitor.

9732. The method of item 9622 wherein the agent is an endothelial growth factor receptor kinase inhibitor.

9733. The method of item 9622 wherein the agent is a retinoic acid receptor antagonist.

9734. The method of item 9622 wherein the agent is a platelet derived growth factor receptor kinase inhibitor.

9735. The method of item 9622 wherein the agent is a fibronogin antagonist.

9736. The method of item 9622 wherein the agent is an antimycotic agent.

9737. The method of item 9622 wherein the agent is an antimycotic agent, wherein the antimycotic agent is sulconizole.

9738. The method of item 9622 wherein the agent is a bisphosphonate.

9739. The method of item 9622 wherein the agent is a phospholipase A1 inhibitor.

9740. The method of item 9622 wherein the agent is a histamine H1/H2/H3 receptor antagonist.

9741. The method of item 9622 wherein the agent is a macrolide antibiotic.

9742. The method of item 9622 wherein the agent is a GPIIb/IIIa receptor antagonist.

9743. The method of item 9622 wherein the agent is an endothelin receptor antagonist.

9744. The method of item 9622 wherein the agent is a peroxisome proliferator-activated receptor agonist.

9745. The method of item 9622 wherein the agent is an estrogen receptor agent.

9746. The method of item 9622 wherein the agent is a somastostatin analogue.

9747. The method of item 9622 wherein the agent is a neurokinin 1 antagonist.

9748. The method of item 9622 wherein the agent is a neurokinin 3 antagonist.

9749. The method of item 9622 wherein the agent is a VLA-4 antagonist.

9750. The method of item 9622 wherein the agent is an osteoclast inhibitor.

9751. The method of item 9622 wherein the agent is a DNA topoisomerase ATP hydrolyzing inhibitor.

9752. The method of item 9622 wherein the agent is an angiotensin I converting enzyme inhibitor.

9753. The method of item 9622 wherein the agent is an angiotensin II antagonist.

9754. The method of item 9622 wherein the agent is an enkephalinase inhibitor.

9755. The method of item 9622 wherein the agent is a peroxisome proliferator-activated receptor gamma agonist insulin sensitizer.

9756. The method of item 9622 wherein the agent is a protein kinase C inhibitor.

9757. The method of item 9622 wherein the agent is a ROCK (rho-associated kinase) inhibitor.

9758. The method of item 9622 wherein the agent is a CXCR3 inhibitor.

9759. The method of item 9622 wherein the agent is an Itk inhibitor.

9760. The method of item 9622 wherein the agent is a cytosolic phospholipase A₂-alpha inhibitor.

9761. The method of item 9622 wherein the agent is a PPAR agonist.

9762. The method of item 9622 wherein the agent is an immunosuppressant.

9763. The method of item 9622 wherein the agent is an Erb inhibitor.

9764. The method of item 9622 wherein the agent is an apoptosis agonist.

9765. The method of item 9622 wherein the agent is a lipocortin agonist.

9766. The method of item 9622 wherein the agent is a VCAM-1 antagonist.

9767. The method of item 9622 wherein the agent is a collagen antagonist.

9768. The method of item 9622 wherein the agent is an alpha 2 integrin antagonist.

9769. The method of item 9622 wherein the agent is a TNF alpha inhibitor.

9770. The method of item 9622 wherein the agent is a nitric oxide inhibitor.

9771. The method of item 9622 wherein the agent is a cathepsin inhibitor.

9772. The method of item 9622 wherein the agent is not an anti-inflammatory agent.

9773. The method of item 9622 wherein the agent is not a steroid.

9774. The method of item 9622 wherein the agent is not a glucocorticosteroid.

9775. The method of item 9622 wherein the agent is not dexamethasone.

9776. The method of item 9622 wherein the agent is not an anti-infective agent.

9777. The method of item 9622 wherein the agent is not an antibiotic.

9778. The method of item 9622 wherein the agent is not an anti-fungal agent.

9779. The method of item 9622, wherein the composition comprises a polymer.

9780. The method of item 9622, wherein the composition comprises a polymer, and the polymer is, or comprises, a copolymer.

9781. The method of item 9622, wherein the composition comprises a polymer, and the polymer is, or comprises, a block copolymer.

9782. The method of item 9622, wherein the composition comprises a polymer, and the polymer is, or comprises, a random copolymer.

9783. The method of item 9622, wherein the composition comprises a polymer, and the polymer is, or comprises, a biodegradable polymer.

9784. The method of item 9622, wherein the composition comprises a polymer, and the polymer is, or comprises, a non-biodegradable polymer.

9785. The method of item 9622, wherein the composition comprises a polymer, and the polymer is, or comprises, a hydrophilic polymer.

9786. The method of item 9622, wherein the composition comprises a polymer, and the polymer is, or comprises, a hydrophobic polymer.

9787. The method of item 9622, wherein the composition comprises a polymer, and the polymer is, or comprises, a polymer having hydrophilic domains.

9788. The method of item 9622, wherein the composition comprises a polymer, and the polymer is, or comprises, a polymer having hydrophobic domains.

9789. The method of item 9622, wherein the composition comprises a polymer, and the polymer is, or comprises, a non-conductive polymer.

9790. The method of item 9622, wherein the composition comprises a polymer, and the polymer is, or comprises, an elastomer.

9791. The method of item 9622, wherein the composition comprises a polymer, and the polymer is, or comprises, a hydrogel.

9792. The method of item 9622, wherein the composition comprises a polymer, and the polymer is, or comprises, a silicone polymer.

9793. The method of item 9622, wherein the composition comprises a polymer, and the polymer is, or comprises, a hydrocarbon polymer.

9794. The method of item 9622, wherein the composition comprises a polymer, and the polymer is, or comprises, a styrene-derived polymer.

9795. The method of item 9622, wherein the composition comprises a polymer, and the polymer is, or comprises, a butadiene-derived polymer.

9796. The method of item 9622, wherein the composition comprises a polymer, and the polymer is, or comprises, a macromer.

9797. The method of item 9622, wherein the composition comprises a polymer, and the polymer is, or comprises, a poly(ethylene glycol)polymer.

9798. The method of item 9622, wherein the composition comprises a polymer, and the polymer is, or comprises, an amorphous polymer.

9799. The method of item 9622, wherein the composition further comprises a second pharmaceutically active agent.

9800. The method of item 9622, wherein the composition further comprises an anti-inflammatory agent.

9801. The method of item 9622, wherein the composition further comprises an agent that inhibits infection.

9802. The method of item 9622, wherein the composition further comprises an anthracycline.

9803. The method of item 9622, wherein the composition further comprises doxorubicin.

9804. The method of item 9622 wherein the composition further comprises mitoxantrone.

9805. The method of item 9622 wherein the composition further comprises a fluoropyrimidine.

9806. The method of item 9622, wherein the composition further comprises 5-fluorouracil (5-FU).

9807. The method of item 9622, wherein the composition further comprises a folic acid antagonist.

9808. The method of item 9622, wherein the composition further comprises methotrexate.

9809. The method of item 9622, wherein the composition further comprises a podophylotoxin.

9810. The method of item 9622, wherein the composition further comprises etoposide.

9811. The method of item 9622, wherein the composition further comprises camptothecin.

9812. The method of item 9622, wherein the composition further comprises a hydroxyurea.

9813. The method of item 9622, wherein the composition further comprises a platinum complex.

9814. The method of item 9622, wherein the composition further comprises cisplatin.

9815. The method of item 9622 wherein the composition further comprises an anti-thrombotic agent.

9816. The method of item 9622, wherein the composition further comprises a visualization agent.

9817. The method of item 9622, wherein the composition further comprises a visualization agent, and the visualization agent is a radiopaque material, wherein the radiopaque material comprises a metal, a halogenated compound, or a barium containing compound.

9818. The method of item 9622, wherein the composition further comprises a visualization agent, and the visualization agent is, or comprises, barium, tantalum, or technetium.

9819. The method of item 9622, wherein the composition further comprises a visualization agent, and the visualization agent is, or comprises, an MRI responsive material.

9820. The method of item 9622, wherein the composition further comprises a visualization agent, and the visualization agent is, or comprises, a gadolinium chelate.

9821. The method of item 9622, wherein the composition further comprises a visualization agent, and the visualization agent is, or comprises, iron, magnesium, manganese, copper, or chromium.

9822. The method of item 9622, wherein the composition further comprises a visualization agent, and the visualization agent is, or comprises, iron oxide compound.

9823. The method of item 9622, wherein the composition further comprises a visualization agent, and the visualization agent is, or comprises, a dye, pigment, or colorant.

9824. The method of item 9622 wherein the agent is released in effective concentrations from the composition comprising the agent by diffusion over a period ranging from the time of administration to about 90 days.

9825. The method of item 9622 wherein the agent is released in effective concentrations from the composition comprising the agent by erosion of the composition over a period ranging from the time of administration to about 90 days.

9826. The method of item 9622 wherein the composition further comprises an inflammatory cytokine.

9827. The method of item 9622 wherein the composition further comprises an agent that stimulates cell proliferation.

9828. The method of item 9622 wherein the composition further comprises a polymeric carrier.

9829. The method of item 9622 wherein the composition is in the form of a gel, paste, or spray.

9830. The method of item 9622 wherein the implant is partially constructed with the agent or the composition.

9831. The method of item 9622 wherein the implant is fully constructed with the agent or the composition.

9832. The method of item 9622 wherein the implant is impregnated with the agent or the composition.

9833. The method of item 9622, wherein the agent or the composition forms a coating, and the coating directly contacts the implant.

9834. The method of item 9622, wherein the agent or the composition forms a coating, and the coating indirectly contacts the implant.

9835. The method of item 9622 wherein the agent or the composition forms a coating, and the coating partially covers the implant.

9836. The method of item 9622, wherein the agent or the composition forms a coating, and the coating completely covers the implant.

9837. The method of item 9622 wherein the agent or the composition is located within pores or holes of the implant.

9838. The method of item 9622 wherein the agent or the composition is located within a channel, lumen, or divet of the implant.

9839. The method of item 9622 wherein the implant further comprising an echogenic material.

9840. The method of item 9622 wherein the implant further comprises an echogenic material, wherein the echogenic material is in the form of a coating.

9841. The method of item 9622 wherein the implant is sterile.

9842. The method of item 9622 wherein the agent is delivered from the implant, wherein the agent is released into tissue in the vicinity of the implant after deployment of the implant.

9843. The method of item 9622 wherein the agent is delivered from the implant, wherein the agent is released into tissue in the vicinity of the implant after deployment of the implant, wherein the tissue is connective tissue.

9844. The method of item 9622 wherein the agent is delivered from the implant, wherein the agent is released into tissue in the vicinity of the implant after deployment of the implant, wherein the tissue is muscle tissue.

9845. The method of item 9622 wherein the agent is delivered from the implant, wherein the agent is released into tissue in the vicinity of the implant after deployment of the implant, wherein the tissue is nerve tissue.

9846. The method of item 9622 wherein the agent is delivered from the implant, wherein the agent is released into tissue in the vicinity of the implant after deployment of the implant, wherein the tissue is epithelium tissue.

9847. The method of item 9622 wherein the agent is delivered from the implant, wherein the agent is released in effective concentrations from the implant over a period ranging from the time of deployment of the implant to about 1 year.

9848. The method of item 9622 wherein the agent is delivered from the implant, wherein the agent is released in effective concentrations from the implant over a period ranging from about 1 month to 6 months.

9849. The method of item 9622 wherein the agent is delivered from the implant, wherein the agent is released in effective concentrations from the implant over a period ranging from about 1-90 days.

9850. The method of item 9622 wherein the agent is delivered from the implant, wherein the agent is released in effective concentrations from the implant at a constant rate.

9851. The method of item 9622 wherein the agent is delivered from the implant, wherein the agent is released in effective concentrations from the implant at an increasing rate.

9852. The method of item 9622 wherein the agent is delivered from the implant, wherein the agent is released in effective concentrations from the implant at a decreasing rate.

9853. The method of item 9622 wherein the agent is delivered from the implant, wherein the implant comprises about 0.01 μg to about 10 μg of the agent.

9854. The method of item 9622 wherein the agent is delivered from the implant, wherein the implant comprises about 10 μg to about 10 mg of the agent.

9855. The method of item 9622 wherein the agent is delivered from the implant, wherein the implant comprises about 10 mg to about 250 mg of the agent.

9856. The method of item 9622 wherein the agent is delivered from the implant, wherein the implant comprises about 250 mg to about 1000 mg of the agent.

9857. The method of item 9622 wherein the agent is delivered from the implant, wherein the implant comprises about 1000 mg to about 2500 mg of the agent.

9858. The method of item 9622 wherein the agent is delivered from the implant, wherein a surface of the implant comprises less than 0.01 μg of the agent per mm² of implant surface to which the agent is applied.

9859. The method of item 9622 wherein the agent is delivered from the implant, wherein a surface of the implant comprises about 0.01 μg to about 1 μg of the agent per mm² of implant surface to which the agent is applied.

9860. The method of item 9622 wherein the agent is delivered from the implant, wherein a surface of the implant comprises about 1 μg to about 10 μg of the agent per mm² of implant surface to which the agent is applied.

9861. The method of item 9622 wherein the agent is delivered from the implant, wherein a surface of the implant comprises about 10 μg to about 250 μg of the agent per mm² of implant surface to which the agent is applied.

9862. The method of item 9622 wherein the agent is delivered from the implant, wherein a surface of the implant comprises about 250 μg to about 1000 μg of the agent per mm² of implant surface to which the agent is applied.

9863. The method of item 9622 wherein the agent is delivered from the implant, wherein a surface of the implant comprises about 1000 μg to about 2500 μg of the agent per mm² of implant surface to which the agent is applied.

9864. The method of item 9622, wherein the implant further comprises a coating, and the coating is a uniform coating.

9865. The method of item 9622, wherein the implant further comprises a coating, and the coating is a non-uniform coating.

9866. The method of item 9622, wherein the implant further comprises a coating, and the coating is a discontinuous coating.

9867. The method of item 9622, wherein the implant further comprises a coating, and the coating is a patterned coating.

9868. The method of item 9622, wherein the implant further comprises a coating, and the coating has a thickness of 100 μm or less.

9869. The method of item 9622, wherein the implant further comprises a coating, and the coating has a thickness of 10 μm or less.

9870. The method of item 9622, wherein the implant further comprises a coating, and the coating adheres to the surface of the implant upon deployment of the implant.

9871. The method of item 9622, wherein the implant further comprises a coating, and the coating is stable at room temperature for a period of at least 1 year.

9872. The method of item 9622, wherein the implant further comprises a coating, and the agent is present in the coating in an amount ranging between about 0.0001% to about 1% by weight.

9873. The method of item 9622, wherein the implant further comprises a coating, and the agent is present in the coating in an amount ranging between about 1% to about 10% by weight.

9874. The method of item 9622, wherein the implant further comprises a coating, and the agent is present in the coating in an amount ranging between about 10% to about 25% by weight.

9875. The method of item 9622, wherein the implant further comprises a coating, and the agent is present in the coating in an amount ranging between about 25% to about 70% by weight.

9876. The method of item 9622, wherein the implant further comprises a coating, and the coating comprises a polymer.

9877. The method of item 9622, wherein the implant comprises a first coating having a first composition and a second coating having a second composition.

9878. The method of item 9622, wherein the implant comprises a first coating having a first composition and a second coating having a second composition, wherein the first composition and the second composition are different.

9879. A method for inhibiting scarring comprising placing a tympanostomy tube implant and an anti-scarring agent or a composition comprising an anti-scarring agent into an animal host, wherein the agent inhibits scarring.

9880. The method of item 9879 wherein the agent inhibits cell regeneration.

9881. The method of item 9879 wherein the agent inhibits angiogenesis.

9882. The method of item 9879 wherein the agent inhibits fibroblast migration.

9883. The method of item 9879 wherein the agent inhibits fibroblast proliferation.

9884. The method of item 9879 wherein the agent inhibits deposition of extracellular matrix.

9885. The method of item 9879 wherein the agent inhibits tissue remodeling.

9886. The method of item 9879 wherein the agent is an angiogenesis inhibitor.

9887. The method of item 9879 wherein the agent is a 5-lipoxygenase inhibitor or antagonist.

9888. The method of item 9879 wherein the agent is a chemokine receptor antagonist.

9889. The method of item 9879 wherein the agent is a cell cycle inhibitor.

9890. The method of item 9879 wherein the agent is a taxane.

9891. The method of item 9879 wherein the agent is an anti-microtubule agent.

9892. The method of item 9879 wherein the agent is paclitaxel.

9893. The method of item 9879 wherein the agent is not paclitaxel.

9894. The method of item 9879 wherein the agent is an analogue or derivative of paclitaxel.

9895. The method of item 9879 wherein the agent is a vinca alkaloid.

9896. The method of item 9879 wherein the agent is camptothecin or an analogue or derivative thereof.

9897. The method of item 9879 wherein the agent is a podophyllotoxin.

9898. The method of item 9879 wherein the agent is a podophyllotoxin, wherein the podophyllotoxin is etoposide or an analogue or derivative thereof.

9899. The method of item 9879 wherein the agent is an anthracycline.

9900. The method of item 9879 wherein the agent is an anthracycline, wherein the anthracycline is doxorubicin or an analogue or derivative thereof.

9901. The method of item 9879 wherein the agent is an anthracycline, wherein the anthracycline is mitoxantrone or an analogue or derivative thereof.

9902. The method of item 9879 wherein the agent is a platinum compound.

9903. The method of item 9879 wherein the agent is a nitrosourea.

9904. The method of item 9879 wherein the agent is a nitroimidazole.

9905. The method of item 9879 wherein the agent is a folic acid antagonist.

9906. The method of item 9879 wherein the agent is a cytidine analogue.

9907. The method of item 9879 wherein the agent is a pyrimidine analogue.

9908. The method of item 9879 wherein the agent is a fluoropyrimidine analogue.

9909. The method of item 9879 wherein the agent is a purine analogue.

9910. The method of item 9879 wherein the agent is a nitrogen mustard or an analogue or derivative thereof.

9911. The method of item 9879 wherein the agent is a hydroxyurea.

9912. The method of item 9879 wherein the agent is a mytomicin or an analogue or derivative thereof.

9913. The method of item 9879 wherein the agent is an alkyl sulfonate.

9914. The method of item 9879 wherein the agent is a benzamide or an analogue or derivative thereof.

9915. The method of item 9879 wherein the agent is a nicotinamide or an analogue or derivative thereof.

9916. The method of item 9879 wherein the agent is a halogenated sugar or an analogue or derivative thereof.

9917. The method of item 9879 wherein the agent is a DNA alkylating agent.

9918. The method of item 9879 wherein the agent is an anti-microtubule agent.

9919. The method of item 9879 wherein the agent is a topoisomerase inhibitor.

9920. The method of item 9879 wherein the agent is a DNA cleaving agent.

9921. The method of item 9879 wherein the agent is an antimetabolite.

9922. The method of item 9879 wherein the agent inhibits adenosine deaminase.

9923. The method of item 9879 wherein the agent inhibits purine ring synthesis.

9924. The method of item 9879 wherein the agent is a nucleotide interconversion inhibitor.

9925. The method of item 9879 wherein the agent inhibits dihydrofolate reduction.

9926. The method of item 9879 wherein the agent blocks thymidine mono phosphate.

9927. The method of item 9879 wherein the agent causes DNA damage.

9928. The method of item 9879 wherein the agent is a DNA intercalation agent.

9929. The method of item 9879 wherein the agent is a RNA synthesis inhibitor.

9930. The method of item 9879 wherein the agent is a pyrimidine synthesis inhibitor.

9931. The method of item 9879 wherein the agent inhibits ribonucleotide synthesis or function.

9932. The method of item 9879 wherein the agent inhibits thymidine monophosphate synthesis or function.

9933. The method of item 9879 wherein the agent inhibits DNA synthesis.

9934. The method of item 9879 wherein the agent causes DNA adduct formation.

9935. The method of item 9879 wherein the agent inhibits protein synthesis.

9936. The method of item 9879 wherein the agent inhibits microtubule function.

9937. The method of item 9879 wherein the agent is a cyclin dependent protein kinase inhibitor.

9938. The method of item 9879 wherein the agent is an epidermal growth factor kinase inhibitor.

9939. The method of item 9879 wherein the agent is an elastase inhibitor.

9940. The method of item 9879 wherein the agent is a factor Xa inhibitor.

9941. The method of item 9879 wherein the agent is a farnesyltransferase inhibitor.

9942. The method of item 9879 wherein the agent is a fibrinogen antagonist.

9943. The method of item 9879 wherein the agent is a guanylate cyclase stimulant.

9944. The method of item 9879 wherein the agent is a heat shock protein 90 antagonist.

9945. The method of item 9879 wherein the agent is a heat shock protein 90 antagonist, wherein the heat shock protein 90 antagonist is geldanamycin or an analogue or derivative thereof.

9946. The method of item 9879 wherein the agent is a guanylate cyclase stimulant.

9947. The method of item 9879 wherein the agent is a HMGCoA reductase inhibitor.

9948. The method of item 9879 wherein the agent is a HMGCoA reductase inhibitor, wherein the HMGCoA reductase inhibitor is simvastatin or an analogue or derivative thereof.

9949. The method of item 9879 wherein the agent is a hydroorotate dehydrogenase inhibitor.

9950. The method of item 9879 wherein the agent is an IKK2 inhibitor.

9951. The method of item 9879 wherein the agent is an IL-1 antagonist.

9952. The method of item 9879 wherein the agent is an ICE antagonist.

9953. The method of item 9879 wherein the agent is an IRAK antagonist.

9954. The method of item 9879 wherein the agent is an IL-4 agonist.

9955. The method of item 9879 wherein the agent is an immunomodulatory agent.

9956. The method of item 9879 wherein the agent is sirolimus or an analogue or derivative thereof.

9957. The method of item 9879 wherein the agent is not sirolimus.

9958. The method of item 9879 wherein the agent is everolimus or an analogue or derivative thereof.

9959. The method of item 9879 wherein the agent is tacrolimus or an analogue or derivative thereof.

9960. The method of item 9879 wherein the agent is not tacrolimus.

9961. The method of item 9879 wherein the agent is biolmus or an analogue or derivative thereof.

9962. The method of item 9879 wherein the agent is tresperimus or an analogue or derivative thereof.

9963. The method of item 9879 wherein the agent is auranofin or an analogue or derivative thereof.

9964. The method of item 9879 wherein the agent is 27-0-demethylrapamycin or an analogue or derivative thereof.

9965. The method of item 9879 wherein the agent is gusperimus or an analogue or derivative thereof.

9966. The method of item 9879 wherein the agent is pimecrolimus or an analogue or derivative thereof.

9967. The method of item 9879 wherein the agent is ABT-578 or an analogue or derivative thereof.

9968. The method of item 9879 wherein the agent is an inosine monophosphate dehydrogenase (IMPDH) inhibitor.

9969. The method of item 9879 wherein the agent is an IMPDH inhibitor, wherein the IMPDH inhibitor is mycophenolic acid or an analogue or derivative thereof.

9970. The method of item 9879 wherein the agent is an IMPDH inhibitor, wherein the IMPDH inhibitor is 1-alpha-25 dihydroxy vitamin D₃ or an analogue or derivative thereof.

9971. The method of item 9879 wherein the agent is a leukotriene inhibitor.

9972. The method of item 9879 wherein the agent is a MCP-1 antagonist.

9973. The method of item 9879 wherein the agent is a MMP inhibitor.

9974. The method of item 9879 wherein the agent is an NF kappa B inhibitor.

9975. The method of item 9879 wherein the agent is an NF kappa B inhibitor, wherein the NF kappa B inhibitor is Bay 11-7082.

9976. The method of item 9879 wherein the agent is an NO agonist.

9977. The method of item 9879 wherein the agent is a p38 MAP kinase inhibitor.

9978. The method of item 9879 wherein the agent is a p38 MAP kinase inhibitor, wherein the p38 MAP kinase inhibitor is SB 202190.

9979. The method of item 9879 wherein the agent is a phosphodiesterase inhibitor.

9980. The method of item 9879 wherein the agent is a TGF beta inhibitor.

9981. The method of item 9879 wherein the agent is a thromboxane A2 antagonist.

9982. The method of item 9879 wherein the agent is a TNFa antagonist.

9983. The method of item 9879 wherein the agent is a TACE inhibitor.

9984. The method of item 9879 wherein the agent is a tyrosine kinase inhibitor.

9985. The method of item 9879 wherein the agent is a vitronectin inhibitor.

9986. The method of item 9879 wherein the agent is a fibroblast growth factor inhibitor.

9987. The method of item 9879 wherein the agent is a protein kinase inhibitor.

9988. The method of item 9879 wherein the agent is a PDGF receptor kinase inhibitor.

9989. The method of item 9879 wherein the agent is an endothelial growth factor receptor kinase inhibitor.

9990. The method of item 9879 wherein the agent is a retinoic acid receptor antagonist.

9991. The method of item 9879 wherein the agent is a platelet derived growth factor receptor kinase inhibitor.

9992. The method of item 9879 wherein the agent is a fibronogin antagonist.

9993. The method of item 9879 wherein the agent is an antimycotic agent.

9994. The method of item 9879 wherein the agent is an antimycotic agent, wherein the antimycotic agent is sulconizole.

9995. The method of item 9879 wherein the agent is a bisphosphonate.

9996. The method of item 9879 wherein the agent is a phospholipase A1 inhibitor.

9997. The method of item 9879 wherein the agent is a histamine H1/H2/H3 receptor antagonist.

9998. The method of item 9879 wherein the agent is a macrolide antibiotic.

9999. The method of item 9879 wherein the agent is a GPIIb/IIIa receptor antagonist.

10000. The method of item 9879 wherein the agent is an endothelin receptor antagonist.

10001. The method of item 9879 wherein the agent is a peroxisome proliferator-activated receptor agonist.

10002. The method of item 9879 wherein the agent is an estrogen receptor agent.

10003. The method of item 9879 wherein the agent is a somastostatin analogue.

10004. The method of item 9879 wherein the agent is a neurokinin 1 antagonist.

10005. The method of item 9879 wherein the agent is a neurokinin 3 antagonist.

10006. The method of item 9879 wherein the agent is a VLA-4 antagonist.

10007. The method of item 9879 wherein the agent is an osteoclast inhibitor.

10008. The method of item 9879 wherein the agent is a DNA topoisomerase ATP hydrolyzing inhibitor.

10009. The method of item 9879 wherein the agent is an angiotensin I converting enzyme inhibitor.

10010. The method of item 9879 wherein the agent is an angiotensin II antagonist.

10011. The method of item 9879 wherein the agent is an enkephalinase inhibitor.

10012. The method of item 9879 wherein the agent is a peroxisome proliferator-activated receptor gamma agonist insulin sensitizer.

10013. The method of item 9879 wherein the agent is a protein kinase C inhibitor.

10014. The method of item 9879 wherein the agent is a ROCK (rho-associated kinase) inhibitor.

10015. The method of item 9879 wherein the agent is a CXCR3 inhibitor.

10016. The method of item 9879 wherein the agent is an ltk inhibitor.

10017. The method of item 9879 wherein the agent is a cytosolic phospholipase A₂-alpha inhibitor.

10018. The method of item 9879 wherein the agent is a PPAR agonist.

10019. The method of item 9879 wherein the agent is an immunosuppressant.

10020. The method of item 9879 wherein the agent is an Erb inhibitor.

10021. The method of item 9879 wherein the agent is an apoptosis agonist.

10022. The method of item 9879 wherein the agent is a lipocortin agonist.

10023. The method of item 9879 wherein the agent is a VCAM-1 antagonist.

10024. The method of item 9879 wherein the agent is a collagen antagonist.

10025. The method of item 9879 wherein the agent is an alpha 2 integrin antagonist.

10026. The method of item 9879 wherein the agent is a TNF alpha inhibitor.

10027. The method of item 9879 wherein the agent is a nitric oxide inhibitor.

10028. The method of item 9879 wherein the agent is a cathepsin inhibitor.

10029. The method of item 9879 wherein the agent is not an anti-inflammatory agent.

10030. The method of item 9879 wherein the agent is not a steroid.

10031. The method of item 9879 wherein the agent is not a glucocorticosteroid.

10032. The method of item 9879 wherein the agent is not dexamethasone.

10033. The method of item 9879 wherein the agent is not an anti-infective agent.

10034. The method of item 9879 wherein the agent is not an antibiotic.

10035. The method of item 9879 wherein the agent is not an anti-fungal agent.

10036. The method of item 9879, wherein the composition comprises a polymer.

10037. The method of item 9879, wherein the composition comprises a polymer, and the polymer is, or comprises, a copolymer.

10038. The method of item 9879, wherein the composition comprises a polymer, and the polymer is, or comprises, a block copolymer.

10039. The method of item 9879, wherein the composition comprises a polymer, and the polymer is, or comprises, a random copolymer.

10040. The method of item 9879, wherein the composition comprises a polymer, and the polymer is, or comprises, a biodegradable polymer.

10041. The method of item 9879, wherein the composition comprises a polymer, and the polymer is, or comprises, a non-biodegradable polymer.

10042. The method of item 9879, wherein the composition comprises a polymer, and the polymer is, or comprises, a hydrophilic polymer.

10043. The method of item 9879, wherein the composition comprises a polymer, and the polymer is, or comprises, a hydrophobic polymer.

10044. The method of item 9879, wherein the composition comprises a polymer, and the polymer is, or comprises, a polymer having hydrophilic domains.

10045. The method of item 9879, wherein the composition comprises a polymer, and the polymer is, or comprises, a polymer having hydrophobic domains.

10046. The method of item 9879, wherein the composition comprises a polymer, and the polymer is, or comprises, a non-conductive polymer.

10047. The method of item 9879, wherein the composition comprises a polymer, and the polymer is, or comprises, an elastomer.

10048. The method of item 9879, wherein the composition comprises a polymer, and the polymer is, or comprises, a hydrogel.

10049. The method of item 9879, wherein the composition comprises a polymer, and the polymer is, or comprises, a silicone polymer.

10050. The method of item 9879, wherein the composition comprises a polymer, and the polymer is, or comprises, a hydrocarbon polymer.

10051. The method of item 9879, wherein the composition comprises a polymer, and the polymer is, or comprises, a styrene-derived polymer.

10052. The method of item 9879, wherein the composition comprises a polymer, and the polymer is, or comprises, a butadiene-derived polymer.

10053. The method of item 9879, wherein the composition comprises a polymer, and the polymer is, or comprises, a macromer.

10054. The method of item 9879, wherein the composition comprises a polymer, and the polymer is, or comprises, a poly(ethylene glycol) polymer.

10055. The method of item 9879, wherein the composition comprises a polymer, and the polymer is, or comprises, an amorphous polymer.

10056. The method of item 9879, wherein the composition further comprises a second pharmaceutically active agent.

10057. The method of item 9879, wherein the composition further comprises an anti-inflammatory agent.

10058. The method of item 9879, wherein the composition further comprises an agent that inhibits infection.

10059. The method of item 9879, wherein the composition further comprises an anthracycline.

10060. The method of item 9879, wherein the composition further comprises doxorubicin.

10061. The method of item 9879 wherein the composition further comprises mitoxantrone.

10062. The method of item 9879 wherein the composition further comprises a fluoropyrimidine.

10063. The method of item 9879, wherein the composition further comprises 5-fluorouracil (5-FU).

10064. The method of item 9879, wherein the composition further comprises a folic acid antagonist.

10065. The method of item 9879, wherein the composition further comprises methotrexate.

10066. The method of item 9879, wherein the composition further comprises a podophylotoxin.

10067. The method of item 9879, wherein the composition further comprises etoposide.

10068. The method of item 9879, wherein the composition further comprises camptothecin.

10069. The method of item 9879, wherein the composition further comprises a hydroxyurea.

10070. The method of item 9879, wherein the composition further comprises a platinum complex.

10071. The method of item 9879, wherein the composition further comprises cisplatin.

10072. The method of item 9879 wherein the composition further comprises an anti-thrombotic agent.

10073. The method of item 9879, wherein the composition further comprises a visualization agent.

10074. The method of item 9879, wherein the composition further comprises a visualization agent, and the visualization agent is a radiopaque material, wherein the radiopaque material comprises a metal, a halogenated compound, or a barium containing compound.

10075. The method of item 9879, wherein the composition further comprises a visualization agent, and the visualization agent is, or comprises, barium, tantalum, or technetium.

10076. The method of item 9879, wherein the composition further comprises a visualization agent, and the visualization agent is, or comprises, an MRI responsive material.

10077. The method of item 9879, wherein the composition further comprises a visualization agent, and the visualization agent is, or comprises, a gadolinium chelate.

10078. The method of item 9879, wherein the composition further comprises a visualization agent, and the visualization agent is, or comprises, iron, magnesium, manganese, copper, or chromium.

10079. The method of item 9879, wherein the composition further comprises a visualization agent, and the visualization agent is, or comprises, iron oxide compound.

10080. The method of item 9879, wherein the composition further comprises a visualization agent, and the visualization agent is, or comprises, a dye, pigment, or colorant.

10081. The method of item 9879 wherein the agent is released in effective concentrations from the composition comprising the agent by diffusion over a period ranging from the time of administration to about 90 days.

10082. The method of item 9879 wherein the agent is released in effective concentrations from the composition comprising the agent by erosion of the composition over a period ranging from the time of administration to about 90 days.

10083. The method of item 9879 wherein the composition further comprises an inflammatory cytokine.

10084. The method of item 9879 wherein the composition further comprises an agent that stimulates cell proliferation.

10085. The method of item 9879 wherein the composition further comprises a polymeric carrier.

10086. The method of item 9879 wherein the composition is in the form of a gel, paste, or spray.

10087. The method of item 9879 wherein the implant is partially constructed with the agent or the composition.

10088. The method of item 9879 wherein the implant is fully constructed with the agent or the composition.

10089. The method of item 9879 wherein the implant is impregnated with the agent or the composition.

10090. The method of item 9879, wherein the agent or the composition forms a coating, and the coating directly contacts the implant.

10091. The method of item 9879, wherein the agent or the composition forms a coating, and the coating indirectly contacts the implant.

10092. The method of item 9879 wherein the agent or the composition forms a coating, and the coating partially covers the implant.

10093. The method of item 9879, wherein the agent or the composition forms a coating, and the coating completely covers the implant.

10094. The method of item 9879 wherein the agent or the composition is located within pores or holes of the implant.

10095. The method of item 9879 wherein the agent or the composition is located within a channel, lumen, or divet of the implant.

10096. The method of item 9879 wherein the implant further comprising an echogenic material.

10097. The method of item 9879 wherein the implant further comprises an echogenic material, wherein the echogenic material is in the form of a coating.

10098. The method of item 9879 wherein the implant is sterile.

10099. The method of item 9879 wherein the agent is delivered from the implant, wherein the agent is released into tissue in the vicinity of the implant after deployment of the implant.

10100. The method of item 9879 wherein the agent is delivered from the implant, wherein the agent is released into tissue in the vicinity of the implant after deployment of the implant, wherein the tissue is connective tissue.

10101. The method of item 9879 wherein the agent is delivered from the implant, wherein the agent is released into tissue in the vicinity of the implant after deployment of the implant, wherein the tissue is muscle tissue.

10102. The method of item 9879 wherein the agent is delivered from the implant, wherein the agent is released into tissue in the vicinity of the implant after deployment of the implant, wherein the tissue is nerve tissue.

10103. The method of item 9879 wherein the agent is delivered from the implant, wherein the agent is released into tissue in the vicinity of the implant after deployment of the implant, wherein the tissue is epithelium tissue.

10104. The method of item 9879 wherein the agent is delivered from the implant, wherein the agent is released in effective concentrations from the implant over a period ranging from the time of deployment of the implant to about 1 year.

10105. The method of item 9879 wherein the agent is delivered from the implant, wherein the agent is released in effective concentrations from the implant over a period ranging from about 1 month to 6 months.

10106. The method of item 9879 wherein the agent is delivered from the implant, wherein the agent is released in effective concentrations from the implant over a period ranging from about 1-90 days.

10107. The method of item 9879 wherein the agent is delivered from the implant, wherein the agent is released in effective concentrations from the implant at a constant rate.

10108. The method of item 9879 wherein the agent is delivered from the implant, wherein the agent is released in effective concentrations from the implant at an increasing rate.

10109. The method of item 9879 wherein the agent is delivered from the implant, wherein the agent is released in effective concentrations from the implant at a decreasing rate.

10110. The method of item 9879 wherein the agent is delivered from the implant, wherein the implant comprises about 0.01 μg to about 10 μg of the agent.

10111. The method of item 9879 wherein the agent is delivered from the implant, wherein the implant comprises about 10 μg to about 10 mg of the agent.

10112. The method of item 9879 wherein the agent is delivered from the implant, wherein the implant comprises about 10 mg to about 250 mg of the agent.

10113. The method of item 9879 wherein the agent is delivered from the implant, wherein the implant comprises about 250 mg to about 1000 mg of the agent.

10114. The method of item 9879 wherein the agent is delivered from the implant, wherein the implant comprises about 1000 mg to about 2500 mg of the agent.

10115. The method of item 9879 wherein the agent is delivered from the implant, wherein a surface of the implant comprises less than 0.01 μg of the agent per mm² of implant surface to which the agent is applied.

10116. The method of item 9879 wherein the agent is delivered from the implant, wherein a surface of the implant comprises about 0.01 μg to about 1 μg of the agent per mm² of implant surface to which the agent is applied.

10117. The method of item 9879 wherein the agent is delivered from the implant, wherein a surface of the implant comprises about 1 μg to about 10 μg of the agent per mm² of implant surface to which the agent is applied.

10118. The method of item 9879 wherein the agent is delivered from the implant, wherein a surface of the implant comprises about 10 μg to about 250 μg of the agent per mm² of implant surface to which the agent is applied.

10119. The method of item 9879 wherein the agent is delivered from the implant, wherein a surface of the implant comprises about 250 μg to about 1000 μg of the agent per mm² of implant surface to which the agent is applied.

10120. The method of item 9879 wherein the agent is delivered from the implant, wherein a surface of the implant comprises about 1000 μg to about 2500 μg of the agent per mm² of implant surface to which the agent is applied.

10121. The method of item 9879, wherein the implant further comprises a coating, and the coating is a uniform coating.

10122. The method of item 9879, wherein the implant further comprises a coating, and the coating is a non-uniform coating.

10123. The method of item 9879, wherein the implant further comprises a coating, and the coating is a discontinuous coating.

10124. The method of item 9879, wherein the implant further comprises a coating, and the coating is a patterned coating.

10125. The method of item 9879, wherein the implant further comprises a coating, and the coating has a thickness of 100 μm or less.

10126. The method of item 9879, wherein the implant further comprises a coating, and the coating has a thickness of 10 μm or less.

10127. The method of item 9879, wherein the implant further comprises a coating, and the coating adheres to the surface of the implant upon deployment of the implant.

10128. The method of item 9879, wherein the implant further comprises a coating, and the coating is stable at room temperature for a period of at least 1 year.

10129. The method of item 9879, wherein the implant further comprises a coating, and the agent is present in the coating in an amount ranging between about 0.0001% to about 1% by weight.

10130. The method of item 9879, wherein the implant further comprises a coating, and the agent is present in the coating in an amount ranging between about 1% to about 10% by weight.

10131. The method of item 9879, wherein the implant further comprises a coating, and the agent is present in the coating in an amount ranging between about 10% to about 25% by weight.

10132. The method of item 9879, wherein the implant further comprises a coating, and the agent is present in the coating in an amount ranging between about 25% to about 70% by weight.

10133. The method of item 9879, wherein the implant further comprises a coating, and the coating comprises a polymer.

10134. The method of item 9879, wherein the implant comprises a first coating having a first composition and a second coating having a second composition.

10135. The method of item 9879, wherein the implant comprises a first coating having a first composition and a second coating having a second composition, wherein the first composition and the second composition are different.

10136. A method for inhibiting scarring comprising placing an implant that provides a surgical adhesion barrier and an anti-scarring agent or a composition comprising an anti-scarring agent into an animal host, wherein the agent inhibits scarring.

10137. The method of item 10136 wherein the agent inhibits cell regeneration.

10138. The method of item 10136 wherein the agent inhibits angiogenesis.

10139. The method of item 10136 wherein the agent inhibits fibroblast migration.

10140. The method of item 10136 wherein the agent inhibits fibroblast proliferation.

10141. The method of item 10136 wherein the agent inhibits deposition of extracellular matrix.

10142. The method of item 10136 wherein the agent inhibits tissue remodeling.

10143. The method of item 10136 wherein the agent is an angiogenesis inhibitor.

10144. The method of item 10136 wherein the agent is a 5-lipoxygenase inhibitor or antagonist.

10145. The method of item 10136 wherein the agent is a chemokine receptor antagonist.

10146. The method of item 10136 wherein the agent is a cell cycle inhibitor.

10147. The method of item 10136 wherein the agent is a taxane.

10148. The method of item 10136 wherein the agent is an anti-microtubule agent.

10149. The method of item 10136 wherein the agent is paclitaxel.

10150. The method of item 10136 wherein the agent is not paclitaxel.

10151. The method of item 10136 wherein the agent is an analogue or derivative of paclitaxel.

10152. The method of item 10136 wherein the agent is a vinca alkaloid.

10153. The method of item 10136 wherein the agent is camptothecin or an analogue or derivative thereof.

10154. The method of item 10136 wherein the agent is a podophyllotoxin.

10155. The method of item 10136 wherein the agent is a podophyllotoxin, wherein the podophyllotoxin is etoposide or an analogue or derivative thereof.

10156. The method of item 10136 wherein the agent is an anthracycline.

10157. The method of item 10136 wherein the agent is an anthracycline, wherein the anthracycline is doxorubicin or an analogue or derivative thereof.

10158. The method of item 10136 wherein the agent is an anthracycline, wherein the anthracycline is mitoxantrone or an analogue or derivative thereof.

10159. The method of item 10136 wherein the agent is a platinum compound.

10160. The method of item 10136 wherein the agent is a nitrosourea.

10161. The method of item 10136 wherein the agent is a nitroimidazole.

10162. The method of item 10136 wherein the agent is a folic acid antagonist.

10163. The method of item 10136 wherein the agent is a cytidine analogue.

10164. The method of item 10136 wherein the agent is a pyrimidine analogue.

10165. The method of item 10136 wherein the agent is a fluoropyrimidine analogue.

10166. The method of item 10136 wherein the agent is a purine analogue.

10167. The method of item 10136 wherein the agent is a nitrogen mustard or an analogue or derivative thereof.

10168. The method of item 10136 wherein the agent is a hydroxyurea.

10169. The method of item 10136 wherein the agent is a mytomicin or an analogue or derivative thereof.

10170. The method of item 10136 wherein the agent is an alkyl sulfonate.

10171. The method of item 10136 wherein the agent is a benzamide or an analogue or derivative thereof.

10172. The method of item 10136 wherein the agent is a nicotinamide or an analogue or derivative thereof.

10173. The method of item 10136 wherein the agent is a halogenated sugar or an analogue or derivative thereof.

10174. The method of item 10136 wherein the agent is a DNA alkylating agent.

10175. The method of item 10136 wherein the agent is an anti-microtubule agent.

10176. The method of item 10136 wherein the agent is a topoisomerase inhibitor.

10177. The method of item 10136 wherein the agent is a DNA cleaving agent.

10178. The method of item 10136 wherein the agent is an antimetabolite.

10179. The method of item 10136 wherein the agent inhibits adenosine deaminase.

10180. The method of item 10136 wherein the agent inhibits purine ring synthesis.

10181. The method of item 10136 wherein the agent is a nucleotide interconversion inhibitor.

10182. The method of item 10136 wherein the agent inhibits dihydrofolate reduction.

10183. The method of item 10136 wherein the agent blocks thymidine monophosphate.

10184. The method of item 10136 wherein the agent causes DNA damage.

10185. The method of item 10136 wherein the agent is a DNA intercalation agent.

10186. The method of item 10136 wherein the agent is a RNA synthesis inhibitor.

10187. The method of item 10136 wherein the agent is a pyrimidine synthesis inhibitor.

10188. The method of item 10136 wherein the agent inhibits ribonucleotide synthesis or function.

10189. The method of item 10136 wherein the agent inhibits thymidine monophosphate synthesis or function.

10190. The method of item 10136 wherein the agent inhibits DNA synthesis.

10191. The method of item 10136 wherein the agent causes DNA adduct formation.

10192. The method of item 10136 wherein the agent inhibits protein synthesis.

10193. The method of item 10136 wherein the agent inhibits microtubule function.

10194. The method of item 10136 wherein the agent is a cyclin dependent protein kinase inhibitor.

10195. The method of item 10136 wherein the agent is an epidermal growth factor kinase inhibitor.

10196. The method of item 10136 wherein the agent is an elastase inhibitor.

10197. The method of item 10136 wherein the agent is a factor Xa inhibitor.

10198. The method of item 10136 wherein the agent is a farnesyltransferase inhibitor.

10199. The method of item 10136 wherein the agent is a fibrinogen antagonist.

10200. The method of item 10136 wherein the agent is a guanylate cyclase stimulant.

10201. The method of item 10136 wherein the agent is a heat shock protein 90 antagonist.

10202. The method of item 10136 wherein the agent is a heat shock protein 90 antagonist, wherein the heat shock protein 90 antagonist is geldanamycin or an analogue or derivative thereof.

10203. The method of item 10136 wherein the agent is a guanylate cyclase stimulant.

10204. The method of item 10136 wherein the agent is a HMGCoA reductase inhibitor.

10205. The method of item 10136 wherein the agent is a HMGCoA reductase inhibitor, wherein the HMGCoA reductase inhibitor is simvastatin or an analogue or derivative thereof.

10206. The method of item 10136 wherein the agent is a hydroorotate dehydrogenase inhibitor.

10207. The method of item 10136 wherein the agent is an IKK2 inhibitor.

10208. The method of item 10136 wherein the agent is an IL-1 antagonist.

10209. The method of item 10136 wherein the agent is an ICE antagonist.

10210. The method of item 10136 wherein the agent is an IRAK antagonist.

10211. The method of item 10136 wherein the agent is an IL-4 agonist.

10212. The method of item 10136 wherein the agent is an immunomodulatory agent.

10213. The method of item 10136 wherein the agent is sirolimus or an analogue or derivative thereof.

10214. The method of item 10136 wherein the agent is not sirolimus.

10215. The method of item 10136 wherein the agent is everolimus or an analogue or derivative thereof.

10216. The method of item 10136 wherein the agent is tacrolimus or an analogue or derivative thereof.

10217. The method of item 10136 wherein the agent is not tacrolimus.

10218. The method of item 10136 wherein the agent is biolmus or an analogue or derivative thereof.

10219. The method of item 10136 wherein the agent is tresperimus or an analogue or derivative thereof.

10220. The method of item 10136 wherein the agent is auranofin or an analogue or derivative thereof.

10221. The method of item 10136 wherein the agent is 27-0-demethylrapamycin or an analogue or derivative thereof.

10222. The method of item 10136 wherein the agent is gusperimus or an analogue or derivative thereof.

10223. The method of item 10136 wherein the agent is pimecrolimus or an analogue or derivative thereof.

10224. The method of item 10136 wherein the agent is ABT-578 or an analogue or derivative thereof.

10225. The method of item 10136 wherein the agent is an inosine monophosphate dehydrogenase (IMPDH) inhibitor.

10226. The method of item 10136 wherein the agent is an IMPDH inhibitor, wherein the IMPDH inhibitor is mycophenolic acid or an analogue or derivative thereof.

10227. The method of item 10136 wherein the agent is an IMPDH inhibitor, wherein the IMPDH inhibitor is 1-alpha-25 dihydroxy vitamin D₃ or an analogue or derivative thereof.

10228. The method of item 10136 wherein the agent is a leukotriene inhibitor.

10229. The method of item 10136 wherein the agent is a MCP-1 antagonist.

10230. The method of item 10136 wherein the agent is a MMP inhibitor.

10231. The method of item 10136 wherein the agent is an NF kappa B inhibitor.

10232. The method of item 10136 wherein the agent is an NF kappa B inhibitor, wherein the NF kappa B inhibitor is Bay 11-7082.

10233. The method of item 10136 wherein the agent is an NO agonist.

10234. The method of item 10136 wherein the agent is a p38 MAP kinase inhibitor.

10235. The method of item 10136 wherein the agent is a p38 MAP kinase inhibitor, wherein the p38 MAP kinase inhibitor is SB 202190.

10236. The method of item 10136 wherein the agent is a phosphodiesterase inhibitor.

10237. The method of item 10136 wherein the agent is a TGF beta inhibitor.

10238. The method of item 10136 wherein the agent is a thromboxane A2 antagonist.

10239. The method of item 10136 wherein the agent is a TNFa antagonist.

10240. The method of item 10136 wherein the agent is a TACE inhibitor.

10241. The method of item 10136 wherein the agent is a tyrosine kinase inhibitor.

10242. The method of item 10136 wherein the agent is a vitronectin inhibitor.

10243. The method of item 10136 wherein the agent is a fibroblast growth factor inhibitor.

10244. The method of item 10136 wherein the agent is a protein kinase inhibitor.

10245. The method of item 10136 wherein the agent is a PDGF receptor kinase inhibitor.

10246. The method of item 10136 wherein the agent is an endothelial growth factor receptor kinase inhibitor.

10247. The method of item 10136 wherein the agent is a retinoic acid receptor antagonist.

10248. The method of item 10136 wherein the agent is a platelet derived growth factor receptor kinase inhibitor.

10249. The method of item 10136 wherein the agent is a fibronogin antagonist.

10250. The method of item 10136 wherein the agent is an antimycotic agent.

10251. The method of item 10136 wherein the agent is an antimycotic agent, wherein the antimycotic agent is sulconizole.

10252. The method of item 10136 wherein the agent is a bisphosphonate.

10253. The method of item 10136 wherein the agent is a phospholipase A1 inhibitor.

10254. The method of item 10136 wherein the agent is a histamine H1/H2/H3 receptor antagonist.

10255. The method of item 10136 wherein the agent is a macrolide antibiotic.

10256. The method of item 10136 wherein the agent is a GPIIb/IIIa receptor antagonist.

10257. The method of item 10136 wherein the agent is an endothelin receptor antagonist.

10258. The method of item 10136 wherein the agent is a peroxisome proliferator-activated receptor agonist.

10259. The method of item 10136 wherein the agent is an estrogen receptor agent.

10260. The method of item 10136 wherein the agent is a somastostatin analogue.

10261. The method of item 10136 wherein the agent is a neurokinin 1 antagonist.

10262. The method of item 10136 wherein the agent is a neurokinin 3 antagonist.

10263. The method of item 10136 wherein the agent is a VLA-4 antagonist.

10264. The method of item 10136 wherein the agent is an osteoclast inhibitor.

10265. The method of item 10136 wherein the agent is a DNA topoisomerase ATP hydrolyzing inhibitor.

10266. The method of item 10136 wherein the agent is an angiotensin I converting enzyme inhibitor.

10267. The method of item 10136 wherein the agent is an angiotensin II antagonist.

10268. The method of item 10136 wherein the agent is an enkephalinase inhibitor.

10269. The method of item 10136 wherein the agent is a peroxisome proliferator-activated receptor gamma agonist insulin sensitizer.

10270. The method of item 10136 wherein the agent is a protein kinase C inhibitor.

10271. The method of item 10136 wherein the agent is a ROCK (rho-associated kinase) inhibitor.

10272. The method of item 10136 wherein the agent is a CXCR3 inhibitor.

10273. The method of item 10136 wherein the agent is an Itk inhibitor.

10274. The method of item 10136 wherein the agent is a cytosolic phospholipase A₂-alpha inhibitor.

10275. The method of item 10136 wherein the agent is a PPAR agonist.

10276. The method of item 10136 wherein the agent is an immunosuppressant.

10277. The method of item 10136 wherein the agent is an Erb inhibitor.

10278. The method of item 10136 wherein the agent is an apoptosis agonist.

10279. The method of item 10136 wherein the agent is a lipocortin agonist.

10280. The method of item 10136 wherein the agent is a VCAM-1 antagonist.

10281. The method of item 10136 wherein the agent is a collagen antagonist.

10282. The method of item 10136 wherein the agent is an alpha 2 integrin antagonist.

10283. The method of item 10136 wherein the agent is a TNF alpha inhibitor.

10284. The method of item 10136 wherein the agent is a nitric oxide inhibitor.

10285. The method of item 10136 wherein the agent is a cathepsin inhibitor.

10286. The method of item 10136 wherein the agent is not an anti-inflammatory agent.

10287. The method of item 10136 wherein the agent is not a steroid.

10288. The method of item 10136 wherein the agent is not a glucocorticosteroid.

10289. The method of item 10136 wherein the agent is not dexamethasone.

10290. The method of item 10136 wherein the agent is not an anti-infective agent.

10291. The method of item 10136 wherein the agent is not an antibiotic.

10292. The method of item 10136 wherein the agent is not an anti-fungal agent.

10293. The method of item 10136, wherein the composition comprises a polymer.

10294. The method of item 10136, wherein the composition comprises a polymer, and the polymer is, or comprises, a copolymer.

10295. The method of item 10136, wherein the composition comprises a polymer, and the polymer is, or comprises, a block copolymer.

10296. The method of item 10136, wherein the composition comprises a polymer, and the polymer is, or comprises, a random copolymer.

10297. The method of item 10136, wherein the composition comprises a polymer, and the polymer is, or comprises, a biodegradable polymer.

10298. The method of item 10136, wherein the composition comprises a polymer, and the polymer is, or comprises, a non-biodegradable polymer.

10299. The method of item 10136, wherein the composition comprises a polymer, and the polymer is, or comprises, a hydrophilic polymer.

10300. The method of item 10136, wherein the composition comprises a polymer, and the polymer is, or comprises, a hydrophobic polymer.

10301. The method of item 10136, wherein the composition comprises a polymer, and the polymer is, or comprises, a polymer having hydrophilic domains.

10302. The method of item 10136, wherein the composition comprises a polymer, and the polymer is, or comprises, a polymer having hydrophobic domains.

10303. The method of item 10136, wherein the composition comprises a polymer, and the polymer is, or comprises, a non-conductive polymer.

10304. The method of item 10136, wherein the composition comprises a polymer, and the polymer is, or comprises, an elastomer.

10305. The method of item 10136, wherein the composition comprises a polymer, and the polymer is, or comprises, a hydrogel.

10306. The method of item 10136, wherein the composition comprises a polymer, and the polymer is, or comprises, a silicone polymer.

10307. The method of item 10136, wherein the composition comprises a polymer, and the polymer is, or comprises, a hydrocarbon polymer.

10308. The method of item 10136, wherein the composition comprises a polymer, and the polymer is, or comprises, a styrene-derived polymer.

10309. The method of item 10136, wherein the composition comprises a polymer, and the polymer is, or comprises, a butadiene-derived polymer.

10310. The method of item 10136, wherein the composition comprises a polymer, and the polymer is, or comprises, a macromer.

10311. The method of item 10136, wherein the composition comprises a polymer, and the polymer is, or comprises, a poly(ethylene glycol) polymer.

10312. The method of item 10136, wherein the composition comprises a polymer, and the polymer is, or comprises, an amorphous polymer.

10313. The method of item 10136, wherein the composition further comprises a second pharmaceutically active agent.

10314. The method of item 10136, wherein the composition further comprises an anti-inflammatory agent.

10315. The method of item 10136, wherein the composition further comprises an agent that inhibits infection.

10316. The method of item 10136, wherein the composition further comprises an anthracycline.

10317. The method of item 10136, wherein the composition further comprises doxorubicin.

10318. The method of item 10136 wherein the composition further comprises mitoxantrone.

10319. The method of item 10136 wherein the composition further comprises a fluoropyrimidine.

10320. The method of item 10136, wherein the composition further comprises 5-fluorouracil (5-FU).

10321. The method of item 10136, wherein the composition further comprises a folic acid antagonist.

10322. The method of item 10136, wherein the composition further comprises methotrexate.

10323. The method of item 10136, wherein the composition further comprises a podophylotoxin.

10324. The method of item 10136, wherein the composition further comprises etoposide.

10325. The method of item 10136, wherein the composition further comprises camptothecin.

10326. The method of item 10136, wherein the composition further comprises a hydroxyurea.

10327. The method of item 10136, wherein the composition further comprises a platinum complex.

10328. The method of item 10136, wherein the composition further comprises cisplatin.

10329. The method of item 10136 wherein the composition further comprises an anti-thrombotic agent.

10330. The method of item 10136, wherein the composition further comprises a visualization agent.

10331. The method of item 10136, wherein the composition further comprises a visualization agent, and the visualization agent is a radiopaque material, wherein the radiopaque material comprises a metal, a halogenated compound, or a barium containing compound.

10332. The method of item 10136, wherein the composition further comprises a visualization agent, and the visualization agent is, or comprises, barium, tantalum, or technetium.

10333. The method of item 10136, wherein the composition further comprises a visualization agent, and the visualization agent is, or comprises, an MRI responsive material.

10334. The method of item 10136, wherein the composition further comprises a visualization agent, and the visualization agent is, or comprises, a gadolinium chelate.

10335. The method of item 10136, wherein the composition further comprises a visualization agent, and the visualization agent is, or comprises, iron, magnesium, manganese, copper, or chromium.

10336. The method of item 10136, wherein the composition further comprises a visualization agent, and the visualization agent is, or comprises, iron oxide compound.

10337. The method of item 10136, wherein the composition further comprises a visualization agent, and the visualization agent is, or comprises, a dye, pigment, or colorant.

10338. The method of item 10136 wherein the agent is released in effective concentrations from the composition comprising the agent by diffusion over a period ranging from the time of administration to about 90 days.

10339. The method of item 10136 wherein the agent is released in effective concentrations from the composition comprising the agent by erosion of the composition over a period ranging from the time of administration to about 90 days.

10340. The method of item 10136 wherein the composition further comprises an inflammatory cytokine.

10341. The method of item 10136 wherein the composition further comprises an agent that stimulates cell proliferation.

10342. The method of item 10136 wherein the composition further comprises a polymeric carrier.

10343. The method of item 10136 wherein the composition is in the form of a gel, paste, or spray.

10344. The method of item 10136 wherein the implant is partially constructed with the agent or the composition.

10345. The method of item 10136 wherein the implant is fully constructed with the agent or the composition.

10346. The method of item 10136 wherein the implant is impregnated with the agent or the composition.

10347. The method of item 10136, wherein the agent or the composition forms a coating, and the coating directly contacts the implant.

10348. The method of item 10136, wherein the agent or the composition forms a coating, and the coating indirectly contacts the implant.

10349. The method of item 10136 wherein the agent or the composition forms a coating, and the coating partially covers the implant.

10350. The method of item 10136, wherein the agent or the composition forms a coating, and the coating completely covers the implant.

10351. The method of item 10136 wherein the agent or the composition is located within pores or holes of the implant.

10352. The method of item 10136 wherein the agent or the composition is located within a channel, lumen, or divet of the implant.

10353. The method of item 10136 wherein the implant further comprising an echogenic material.

10354. The method of item 10136 wherein the implant further comprises an echogenic material, wherein the echogenic material is in the form of a coating.

10355. The method of item 10136 wherein the implant is sterile.

10356. The method of item 10136 wherein the agent is delivered from the implant, wherein the agent is released into tissue in the vicinity of the implant after deployment of the implant.

10357. The method of item 10136 wherein the agent is delivered from the implant, wherein the agent is released into tissue in the vicinity of the implant after deployment of the implant, wherein the tissue is connective tissue.

10358. The method of item 10136 wherein the agent is delivered from the implant, wherein the agent is released into tissue in the vicinity of the implant after deployment of the implant, wherein the tissue is muscle tissue.

10359. The method of item 10136 wherein the agent is delivered from the implant, wherein the agent is released into tissue in the vicinity of the implant after deployment of the implant, wherein the tissue is nerve tissue.

10360. The method of item 10136 wherein the agent is delivered from the implant, wherein the agent is released into tissue in the vicinity of the implant after deployment of the implant, wherein the tissue is epithelium tissue.

10361. The method of item 10136 wherein the agent is delivered from the implant, wherein the agent is released in effective concentrations from the implant over a period ranging from the time of deployment of the implant to about 1 year.

10362. The method of item 10136 wherein the agent is delivered from the implant, wherein the agent is released in effective concentrations from the implant over a period ranging from about 1 month to 6 months.

10363. The method of item 10136 wherein the agent is delivered from the implant, wherein the agent is released in effective concentrations from the implant over a period ranging from about 1-90 days.

10364. The method of item 10136 wherein the agent is delivered from the implant, wherein the agent is released in effective concentrations from the implant at a constant rate.

10365. The method of item 10136 wherein the agent is delivered from the implant, wherein the agent is released in effective concentrations from the implant at an increasing rate.

10366. The method of item 10136 wherein the agent is delivered from the implant, wherein the agent is released in effective concentrations from the implant at a decreasing rate.

10367. The method of item 10136 wherein the agent is delivered from the implant, wherein the implant comprises about 0.01 μg to about 10 μg of the agent.

10368. The method of item 10136 wherein the agent is delivered from the implant, wherein the implant comprises about 10 μg to about 10 mg of the agent.

10369. The method of item 10136 wherein the agent is delivered from the implant, wherein the implant comprises about 10 mg to about 250 mg of the agent.

10370. The method of item 10136 wherein the agent is delivered from the implant, wherein the implant comprises about 250 mg to about 1000 mg of the agent.

10371. The method of item 10136 wherein the agent is delivered from the implant, wherein the implant comprises about 1000 mg to about 2500 mg of the agent.

10372. The method of item 10136 wherein the agent is delivered from the implant, wherein a surface of the implant comprises less than 0.01 μg of the agent per mm² of implant surface to which the agent is applied.

10373. The method of item 10136 wherein the agent is delivered from the implant, wherein a surface of the implant comprises about 0.01 μg to about 1 μg of the agent per mm² of implant surface to which the agent is applied.

10374. The method of item 10136 wherein the agent is delivered from the implant, wherein a surface of the implant comprises about 1 μg to about 10 μg of the agent per mm² of implant surface to which the agent is applied.

10375. The method of item 10136 wherein the agent is delivered from the implant, wherein a surface of the implant comprises about 10 μg to about 250 μg of the agent per mm² of implant surface to which the agent is applied.

10376. The method of item 10136 wherein the agent is delivered from the implant, wherein a surface of the implant comprises about 250 μg to about 1000 μg of the agent per mm² of implant surface to which the agent is applied.

10377. The method of item 10136 wherein the agent is delivered from the implant, wherein a surface of the implant comprises about 1000 μg to about 2500 μg of the agent per mm² of implant surface to which the agent is applied.

10378. The method of item 10136, wherein the implant further comprises a coating, and the coating is a uniform coating.

10379. The method of item 10136, wherein the implant further comprises a coating, and the coating is a non-uniform coating.

10380. The method of item 10136, wherein the implant further comprises a coating, and the coating is a discontinuous coating.

10381. The method of item 10136, wherein the implant further comprises a coating, and the coating is a patterned coating.

10382. The method of item 10136, wherein the implant further comprises a coating, and the coating has a thickness of 100 μm or less.

10383. The method of item 10136, wherein the implant further comprises a coating, and the coating has a thickness of 10 μm or less.

10384. The method of item 10136, wherein the implant further comprises a coating, and the coating adheres to the surface of the implant upon deployment of the implant.

10385. The method of item 10136, wherein the implant further comprises a coating, and the coating is stable at room temperature for a period of at least 1 year.

10386. The method of item 10136, wherein the implant further comprises a coating, and the agent is present in the coating in an amount ranging between about 0.0001% to about 1% by weight.

10387. The method of item 10136, wherein the implant further comprises a coating, and the agent is present in the coating in an amount ranging between about 1% to about 10% by weight.

10388. The method of item 10136, wherein the implant further comprises a coating, and the agent is present in the coating in an amount ranging between about 10% to about 25% by weight.

10389. The method of item 10136, wherein the implant further comprises a coating, and the agent is present in the coating in an amount ranging between about 25% to about 70% by weight.

10390. The method of item 10136, wherein the implant further comprises a coating, and the coating comprises a polymer.

10391. The method of item 10136, wherein the implant comprises a first coating having a first composition and a second coating having a second composition.

10392. The method of item 10136, wherein the implant comprises a first coating having a first composition and a second coating having a second composition, wherein the first composition and the second composition are different.

10393. A composition comprising surgical adhesion barrier components and an anti-scarring agent, wherein the composition inhibits formation of surgical adhesions, and wherein the agent inhibits scarring in the vicinity of the composition as it is located within a host that has received the composition.

10394. The composition of item 10393 wherein the agent inhibits cell regeneration.

10395. The composition of item 10393 wherein the agent inhibits angiogenesis.

10396. The composition of item 10393 wherein the agent inhibits fibroblast migration.

10397. The composition of item 10393 wherein the agent inhibits fibroblast proliferation.

10398. The composition of item 10393 wherein the agent inhibits deposition of extracellular matrix.

10399. The composition of item 10393 wherein the agent inhibits tissue remodeling.

10400. The composition of item 10393 wherein the agent is an angiogenesis inhibitor.

10401. The composition of item 10393 wherein the agent is a 5-lipoxygenase inhibitor or antagonist.

10402. The composition of item 10393 wherein the agent is a chemokine receptor antagonist.

10403. The composition of item 10393 wherein the agent is a cell cycle inhibitor.

10404. The composition of item 10393 wherein the agent is a taxane.

10405. The composition of item 10393 wherein the agent is an anti-microtubule agent.

10406. The composition of item 10393 wherein the agent is paclitaxel.

10407. The composition of item 10393 wherein the agent is not paclitaxel.

10408. The composition of item 10393 wherein the agent is an analogue or derivative of paclitaxel.

10409. The composition of item 10393 wherein the agent is a vinca alkaloid.

10410. The composition of item 10393 wherein the agent is camptothecin or an analogue or derivative thereof.

10411. The composition of item 10393 wherein the agent is a podophyllotoxin.

10412. The composition of item 10393 wherein the agent is a podophyllotoxin, wherein the podophyllotoxin is etoposide or an analogue or derivative thereof.

10413. The composition of item 10393 wherein the agent is an anthracycline.

10414. The composition of item 10393 wherein the agent is an anthracycline, wherein the anthracycline is doxorubicin or an analogue or derivative thereof.

10415. The composition of item 10393 wherein the agent is an anthracycline, wherein the anthracycline is mitoxantrone or an analogue or derivative thereof.

10416. The composition of item 10393 wherein the agent is a platinum compound.

10417. The composition of item 10393 wherein the agent is a nitrosourea.

10418. The composition of item 10393 wherein the agent is a nitroimidazole.

10419. The composition of item 10393 wherein the agent is a folic acid antagonist.

10420. The composition of item 10393 wherein the agent is a cytidine analogue.

10421. The composition of item 10393 wherein the agent is a pyrimidine analogue.

10422. The composition of item 10393 wherein the agent is a fluoropyrimidine analogue.

10423. The composition of item 10393 wherein the agent is a purine analogue.

10424. The composition of item 10393 wherein the agent is a nitrogen mustard or an analogue or derivative thereof.

10425. The composition of item 10393 wherein the agent is a hydroxyurea.

10426. The composition of item 10393 wherein the agent is a mytomicin or an analogue or derivative thereof.

10427. The composition of item 10393 wherein the agent is an alkyl sulfonate.

10428. The composition of item 10393 wherein the agent is a benzamide or an analogue or derivative thereof.

10429. The composition of item 10393 wherein the agent is a nicotinamide or an analogue or derivative thereof.

10430. The composition of item 10393 wherein the agent is a halogenated sugar or an analogue or derivative thereof.

10431. The composition of item 10393 wherein the agent is a DNA alkylating agent.

10432. The composition of item 10393 wherein the agent is an anti-microtubule agent.

10433. The composition of item 10393 wherein the agent is a topoisomerase inhibitor.

10434. The composition of item 10393 wherein the agent is a DNA cleaving agent.

10435. The composition of item 10393 wherein the agent is an antimetabolite.

10436. The composition of item 10393 wherein the agent inhibits adenosine deaminase.

10437. The composition of item 10393 wherein the agent inhibits purine ring synthesis.

10438. The composition of item 10393 wherein the agent is a nucleotide interconversion inhibitor.

10439. The composition of item 10393 wherein the agent inhibits dihydrofolate reduction.

10440. The composition of item 10393 wherein the agent blocks thymidine monophosphate.

10441. The composition of item 10393 wherein the agent causes DNA damage.

10442. The composition of item 10393 wherein the agent is a DNA intercalation agent.

10443. The composition of item 10393 wherein the agent is a RNA synthesis inhibitor.

10444. The composition of item 10393 wherein the agent is a pyrimidine synthesis inhibitor.

10445. The composition of item 10393 wherein the agent inhibits ribonucleotide synthesis or function.

10446. The composition of item 10393 wherein the agent inhibits thymidine monophosphate synthesis or function.

10447. The composition of item 10393 wherein the agent inhibits DNA synthesis.

10448. The composition of item 10393 wherein the agent causes DNA adduct formation.

10449. The composition of item 10393 wherein the agent inhibits protein synthesis.

10450. The composition of item 10393 wherein the agent inhibits microtubule function.

10451. The composition of item 10393 wherein the agent is a cyclin dependent protein kinase inhibitor.

10452. The composition of item 10393 wherein the agent is an epidermal growth factor kinase inhibitor.

10453. The composition of item 10393 wherein the agent is an elastase inhibitor.

10454. The composition of item 10393 wherein the agent is a factor Xa inhibitor.

10455. The composition of item 10393 wherein the agent is a farnesyltransferase inhibitor.

10456. The composition of item 10393 wherein the agent is a fibrinogen antagonist.

10457. The composition of item 10393 wherein the agent is a guanylate cyclase stimulant.

10458. The composition of item 10393 wherein the agent is a heat shock protein 90 antagonist.

10459. The composition of item 10393 wherein the agent is a heat shock protein 90 antagonist, wherein the heat shock protein 90 antagonist is geldanamycin or an analogue or derivative thereof.

10460. The composition of item 10393 wherein the agent is a guanylate cyclase stimulant.

10461. The composition of item 10393 wherein the agent is a HMGCoA reductase inhibitor.

10462. The composition of item 10393 wherein the agent is a HMGCoA reductase inhibitor, wherein the HMGCoA reductase inhibitor is simvastatin or an analogue or derivative thereof.

10463. The composition of item 10393 wherein the agent is a hydroorotate dehydrogenase inhibitor.

10464. The composition of item 10393 wherein the agent is an IKK2 inhibitor.

10465. The composition of item 10393 wherein the agent is an IL-1 antagonist.

10466. The composition of item 10393 wherein the agent is an ICE antagonist.

10467. The composition of item 10393 wherein the agent is an IRAK antagonist.

10468. The composition of item 10393 wherein the agent is an IL-4 agonist.

10469. The composition of item 10393 wherein the agent is an immunomodulatory agent.

10470. The composition of item 10393 wherein the agent is sirolimus or an analogue or derivative thereof.

10471. The composition of item 10393 wherein the agent is not sirolimus.

10472. The composition of item 10393 wherein the agent is everolimus or an analogue or derivative thereof.

10473. The composition of item 10393 wherein the agent is tacrolimus or an analogue or derivative thereof.

10474. The composition of item 10393 wherein the agent is not tacrolimus.

10475. The composition of item 10393 wherein the agent is biolmus or an analogue or derivative thereof.

10476. The composition of item 10393 wherein the agent is tresperimus or an analogue or derivative thereof.

10477. The composition of item 10393 wherein the agent is auranofin or an analogue or derivative thereof.

10478. The composition of item 10393 wherein the agent is 27-O-demethylrapamycin or an analogue or derivative thereof.

10479. The composition of item 10393 wherein the agent is gusperimus or an analogue or derivative thereof.

10480. The composition of item 10393 wherein the agent is pimecrolimus or an analogue or derivative thereof.

10481. The composition of item 10393 wherein the agent is ABT-578 or an analogue or derivative thereof.

10482. The composition of item 10393 wherein the agent is an inosine monophosphate dehydrogenase (IMPDH) inhibitor.

10483. The composition of item 10393 wherein the agent is an IMPDH inhibitor, wherein the IMPDH inhibitor is mycophenolic acid or an analogue or derivative thereof.

10484. The composition of item 10393 wherein the agent is an IMPDH inhibitor, wherein the IMPDH inhibitor is 1-alpha-25 dihydroxy vitamin D3 or an analogue or derivative thereof.

10485. The composition of item 10393 wherein the agent is a leukotriene inhibitor.

10486. The composition of item 10393 wherein the agent is a MCP-1 antagonist.

10487. The composition of item 10393 wherein the agent is a MMP inhibitor.

10488. The composition of item 10393 wherein the agent is an NF kappa B inhibitor.

10489. The composition of item 10393 wherein the agent is an NF kappa B inhibitor, wherein the NF kappa B inhibitor is Bay 11-7082.

10490. The composition of item 10393 wherein the agent is an NO agonist.

10491. The composition of item 10393 wherein the agent is a p38 MAP kinase inhibitor.

10492. The composition of item 10393 wherein the agent is a p38 MAP kinase inhibitor, wherein the p38 MAP kinase inhibitor is SB 202190.

10493. The composition of item 10393 wherein the agent is a phosphodiesterase inhibitor.

10494. The composition of item 10393 wherein the agent is a TGF beta inhibitor.

10495. The composition of item 10393 wherein the agent is a thromboxane A2 antagonist.

10496. The composition of item 10393 wherein the agent is a TNFa antagonist.

10497. The composition of item 10393 wherein the agent is a TACE inhibitor.

10498. The composition of item 10393 wherein the agent is a tyrosine kinase inhibitor.

10499. The composition of item 10393 wherein the agent is a vitronectin inhibitor.

10500. The composition of item 10393 wherein the agent is a fibroblast growth factor inhibitor.

10501. The composition of item 10393 wherein the agent is a protein kinase inhibitor.

10502. The composition of item 10393 wherein the agent is a PDGF receptor kinase inhibitor.

10503. The composition of item 10393 wherein the agent is an endothelial growth factor receptor kinase inhibitor.

10504. The composition of item 10393 wherein the agent is a retinoic acid receptor antagonist.

10505. The composition of item 10393 wherein the agent is a platelet derived growth factor receptor kinase inhibitor.

10506. The composition of item 10393 wherein the agent is a fibronogin antagonist.

10507. The composition of item 10393 wherein the agent is an antimycotic agent.

10508. The composition of item 10393 wherein the agent is an antimycotic agent, wherein the antimycotic agent is sulconizole.

10509. The composition of item 10393 wherein the agent is a bisphosphonate.

10510. The composition of item 10393 wherein the agent is a phospholipase A1 inhibitor.

10511. The composition of item 10393 wherein the agent is a histamine H1/H2/H3 receptor antagonist.

10512. The composition of item 10393 wherein the agent is a macrolide antibiotic.

10513. The composition of item 10393 wherein the agent is a GPIIb/IIIa receptor antagonist.

10514. The composition of item 10393 wherein the agent is an endothelin receptor antagonist.

10515. The composition of item 10393 wherein the agent is a peroxisome proliferator-activated receptor agonist.

10516. The composition of item 10393 wherein the agent is an estrogen receptor agent.

10517. The composition of item 10393 wherein the agent is a somastostatin analogue.

10518. The composition of item 10393 wherein the agent is a neurokinin 1 antagonist.

10519. The composition of item 10393 wherein the agent is a neurokinin 3 antagonist.

10520. The composition of item 10393 wherein the agent is a VLA-4 antagonist.

10521. The composition of item 10393 wherein the agent is an osteoclast inhibitor.

10522. The composition of item 10393 wherein the agent is a DNA topoisomerase ATP hydrolyzing inhibitor.

10523. The composition of item 10393 wherein the agent is an angiotensin I converting enzyme inhibitor.

10524. The composition of item 10393 wherein the agent is an angiotensin II antagonist.

10525. The composition of item 10393 wherein the agent is an enkephalinase inhibitor.

10526. The composition of item 10393 wherein the agent is a peroxisome proliferator-activated receptor gamma agonist insulin sensitizer.

10527. The composition of item 10393 wherein the agent is a protein kinase C inhibitor.

10528. The composition of item 10393 wherein the agent is a ROCK (rho-associated kinase) inhibitor.

10529. The composition of item 10393 wherein the agent is a CXCR3 inhibitor.

10530. The composition of item 10393 wherein the agent is an ltk inhibitor.

10531. The composition of item 10393 wherein the agent is a cytosolic phospholipase A2-alpha inhibitor.

10532. The composition of item 10393 wherein the agent is a PPAR agonist.

10533. The composition of item 10393 wherein the agent is an immunosuppressant.

10534. The composition of item 10393 wherein the agent is an Erb inhibitor.

10535. The composition of item 10393 wherein the agent is an apoptosis agonist.

10536. The composition of item 10393 wherein the agent is a lipocortin agonist.

10537. The composition of item 10393 wherein the agent is a VCAM-1 antagonist.

10538. The composition of item 10393 wherein the agent is a collagen antagonist.

10539. The composition of item 10393 wherein the agent is an alpha 2 integrin antagonist.

10540. The composition of item 10393 wherein the agent is a TNF alpha inhibitor.

10541. The composition of item 10393 wherein the agent is a nitric oxide inhibitor.

10542. The composition of item 10393 wherein the agent is a cathepsin inhibitor.

10543. The composition of item 10393 wherein the agent is not an anti-inflammatory agent.

10544. The composition of item 10393 wherein the agent is not a steroid.

10545. The composition of item 10393 wherein the agent is not a glucocorticosteroid.

10546. The composition of item 10393 wherein the agent is not dexamethasone.

10547. The composition of item 10393 wherein the agent is not an anti-infective agent.

10548. The composition of item 10393 wherein the agent is not an antibiotic.

10549. The composition of item 10393 wherein the agent is not an anti-fungal agent.

10550. The composition of item 10393, further comprising a polymer.

10551. The composition of item 10393, further comprising a polymeric carrier.

10552. The composition of item 10393, further comprising a second pharmaceutically active agent.

10553. The composition of item 10393, further comprising an anti-inflammatory agent.

10554. The composition of item 10393, further comprising an agent that inhibits infection.

10555. The composition of item 10393, further comprising an agent that inhibits infection, wherein the agent is an anthracycline.

10556. The composition of item 10393, further comprising an agent that inhibits infection, wherein the agent is doxorubicin.

10557. The composition of item 10393, further comprising an agent that inhibits infection, wherein the agent is mitoxantrone.

10558. The composition of item 10393, further comprising an agent that inhibits infection, wherein the agent is a fluoropyrimidine.

10559. The composition of item 10393, further comprising an agent that inhibits infection, wherein the agent is 5-fluorouracil (5-FU).

10560. The composition of item 10393, further comprising an agent that inhibits infection, wherein the agent is a folic acid antagonist.

10561. The composition of item 10393, further comprising an agent that inhibits infection, wherein the agent is methotrexate.

10562. The composition of item 10393, further comprising an agent that inhibits infection, wherein the agent is a podophylotoxin.

10563. The composition of item 10393, further comprising an agent that inhibits infection, wherein the agent is etoposide.

10564. The composition of item 10393, further comprising an agent that inhibits infection, wherein the agent is a camptothecin.

10565. The composition of item 10393, further comprising an agent that inhibits infection, wherein the agent is a hydroxyurea.

10566. The composition of item 10393, further comprising an agent that inhibits infection, wherein the agent is a platinum complex.

10567. The composition of item 10393, further comprising an agent that inhibits infection, wherein the agent is cisplatin.

10568. The composition of item 10393, further comprising an anti-thrombotic agent.

10569. The composition of item 10393, further comprising a visualization agent.

10570. The composition of item 10393, further comprising a visualization agent, wherein the visualization agent is a radiopaque material, wherein the radiopaque material comprises a metal, a halogenated compound, or a barium containing compound.

10571. The composition of item 10393, further comprising a visualization agent, wherein the visualization agent is a radiopaque material, wherein the radiopaque material comprises barium, tantalum, or technetium.

10572. The composition of item 10393, further comprising a visualization agent, wherein the visualization agent is a MRI responsive material.

10573. The composition of item 10393, further comprising a visualization agent, wherein the visualization agent comprises a gadolinium chelate.

10574. The composition of item 10393, further comprising a visualization agent, wherein the visualization agent comprises iron, magnesium, manganese, copper, or chromium.

10575. The composition of item 10393, further comprising a visualization agent, wherein the visualization agent comprises an iron oxide compound.

10576. The composition of item 10393, further comprising a visualization agent, wherein the visualization agent comprises a dye, pigment, or colorant.

10577. The composition of item 10393, further comprising an echogenic material.

10578. The composition of item 10393 wherein the components comprise hyaluronic acid or an analog or derivative thereof.

10579. The composition of items 10393 wherein the components form a biodegradable polymeric matrix when the composition is administered to the host.

10580. The composition of items 10393 in a sprayable form.

10581. The composition of items 10393 in a gel form.

10582. The composition of items 10393 wherein the components have reacted to form a film.

10583. The composition of items 10393 in the form of a film.

10584. The composition of items 10393 wherein the components have reacted to form a wrap.

10585. The composition of items 10393 in the form of a wrap.

10586. The composition of items 10393 wherein the components have reacted to form a mesh.

10587. The composition of items 10393 in the form of a mesh.

10588. The composition of items 10393 wherein the components comprise hyaluronic acid or an analog or derivative thereof.

10589. A method of making a medical device comprising: combining an intravascular implant and an anti-scarring agent or a composition comprising an anti-scarring agent, wherein the agent inhibits scarring between the device and a host into which the device is implanted.

10590. The method of item 10589 wherein the agent inhibits cell regeneration.

10591. The method of item 10589 wherein the agent inhibits angiogenesis.

10592. The method of item 10589 wherein the agent inhibits fibroblast migration.

10593. The method of item 10589 wherein the agent inhibits fibroblast proliferation.

10594. The method of item 10589 wherein the agent inhibits deposition of extracellular matrix.

10595. The method of item 10589 wherein the agent inhibits tissue remodeling.

10596. The method of item 10589 wherein the agent is an angiogenesis inhibitor.

10597. The method of item 10589 wherein the agent is a 5-lipoxygenase inhibitor or antagonist.

10598. The method of item 10589 wherein the agent is a chemokine receptor antagonist.

10599. The method of item 10589 wherein the agent is a cell cycle inhibitor.

10600. The method of item 10589 wherein the agent is a taxane.

10601. The method of item 10589 wherein the agent is an anti-microtubule agent.

10602. The method of item 10589 wherein the agent is paclitaxel.

10603. The method of item 10589 wherein the agent is not paclitaxel.

10604. The method of item 10589 wherein the agent is an analogue or derivative of paclitaxel.

10605. The method of item 10589 wherein the agent is a vinca alkaloid.

10606. The method of item 10589 wherein the agent is camptothecin or an analogue or derivative thereof.

10607. The method of item 10589 wherein the agent is a podophyllotoxin.

10608. The method of item 10589 wherein the agent is a podophyllotoxin, wherein the podophyllotoxin is etoposide or an analogue or derivative thereof.

10609. The method of item 10589 wherein the agent is an anthracycline.

10610. The method of item 10589 wherein the agent is an anthracycline, wherein the anthracycline is doxorubicin or an analogue or derivative thereof.

10611. The method of item 10589 wherein the agent is an anthracycline, wherein the anthracycline is mitoxantrone or an analogue or derivative thereof.

10612. The method of item 10589 wherein the agent is a platinum compound.

10613. The method of item 10589 wherein the agent is a nitrosourea.

10614. The method of item 10589 wherein the agent is a nitroimidazole.

10615. The method of item 10589 wherein the agent is a folic acid antagonist.

10616. The method of item 10589 wherein the agent is a cytidine analogue.

10617. The method of item 10589 wherein the agent is a pyrimidine analogue.

10618. The method of item 10589 wherein the agent is a fluoropyrimidine analogue.

10619. The method of item 10589 wherein the agent is a purine analogue.

10620. The method of item 10589 wherein the agent is a nitrogen mustard or an analogue or derivative thereof.

10621. The method of item 10589 wherein the agent is a hydroxyurea.

10622. The method of item 10589 wherein the agent is a mytomicin or an analogue or derivative thereof.

10623. The method of item 10589 wherein the agent is an alkyl sulfonate.

10624. The method of item 10589 wherein the agent is a benzamide or an analogue or derivative thereof.

10625. The method of item 10589 wherein the agent is a nicotinamide or an analogue or derivative thereof.

10626. The method of item 10589 wherein the agent is a halogenated sugar or an analogue or derivative thereof.

10627. The method of item 10589 wherein the agent is a DNA alkylating agent.

10628. The method of item 10589 wherein the agent is an anti-microtubule agent.

10629. The method of item 10589 wherein the agent is a topoisomerase inhibitor.

10630. The method of item 10589 wherein the agent is a DNA cleaving agent.

10631. The method of item 10589 wherein the agent is an antimetabolite.

10632. The method of item 10589 wherein the agent inhibits adenosine deaminase.

10633. The method of item 10589 wherein the agent inhibits purine ring synthesis.

10634. The method of item 10589 wherein the agent is a nucleotide interconversion inhibitor.

10635. The method of item 10589 wherein the agent inhibits dihydrofolate reduction.

10636. The method of item 10589 wherein the agent blocks thymidine monophosphate.

10637. The method of item 10589 wherein the agent causes DNA damage.

10638. The method of item 10589 wherein the agent is a DNA intercalation agent.

10639. The method of item 10589 wherein the agent is a RNA synthesis inhibitor.

10640. The method of item 10589 wherein the agent is a pyrimidine synthesis inhibitor.

10641. The method of item 10589 wherein the agent inhibits ribonucleotide synthesis or function.

10642. The method of item 10589 wherein the agent inhibits thymidine monophosphate synthesis or function.

10643. The method of item 10589 wherein the agent inhibits DNA synthesis.

10644. The method of item 10589 wherein the agent causes DNA adduct formation.

10645. The method of item 10589 wherein the agent inhibits protein synthesis.

10646. The method of item 10589 wherein the agent inhibits microtubule function.

10647. The method of item 10589 wherein the agent is a cyclin dependent protein kinase inhibitor.

10648. The method of item 10589 wherein the agent is an epidermal growth factor kinase inhibitor.

10649. The method of item 10589 wherein the agent is an elastase inhibitor.

10650. The method of item 10589 wherein the agent is a factor Xa inhibitor.

10651. The method of item 10589 wherein the agent is a farnesyltransferase inhibitor.

10652. The method of item 10589 wherein the agent is a fibrinogen antagonist.

10653. The method of item 10589 wherein the agent is a guanylate cyclase stimulant.

10654. The method of item 10589 wherein the agent is a heat shock protein 90 antagonist.

10655. The method of item 10589 wherein the agent is a heat shock protein 90 antagonist, wherein the heat shock protein 90 antagonist is geldanamycin or an analogue or derivative thereof.

10656. The method of item 10589 wherein the agent is a guanylate cyclase stimulant.

10657. The method of item 10589 wherein the agent is a HMGCoA reductase inhibitor.

10658. The method of item 10589 wherein the agent is a HMGCoA reductase inhibitor, wherein the HMGCoA reductase inhibitor is simvastatin or an analogue or derivative thereof.

10659. The method of item 10589 wherein the agent is a hydroorotate dehydrogenase inhibitor.

10660. The method of item 10589 wherein the agent is an IKK2 inhibitor.

10661. The method of item 10589 wherein the agent is an IL-1 antagonist.

10662. The method of item 10589 wherein the agent is an ICE antagonist.

10663. The method of item 10589 wherein the agent is an IRAK antagonist.

10664. The method of item 10589 wherein the agent is an IL-4 agonist.

10665. The method of item 10589 wherein the agent is an immunomodulatory agent.

10666. The method of item 10589 wherein the agent is sirolimus or an analogue or derivative thereof.

10667. The method of item 10589 wherein the agent is not sirolimus.

10668. The method of item 10589 wherein the agent is everolimus or an analogue or derivative thereof.

10669. The method of item 10589 wherein the agent is tacrolimus or an analogue or derivative thereof.

10670. The method of item 10589 wherein the agent is not tacrolimus.

10671. The method of item 10589 wherein the agent is biolmus or an analogue or derivative thereof.

10672. The method of item 10589 wherein the agent is tresperimus or an analogue or derivative thereof.

10673. The method of item 10589 wherein the agent is auranofin or an analogue or derivative thereof.

10674. The method of item 10589 wherein the agent is 27-0-demethylrapamycin or an analogue or derivative thereof.

10675. The method of item 10589 wherein the agent is gusperimus or an analogue or derivative thereof.

10676. The method of item 10589 wherein the agent is pimecrolimus or an analogue or derivative thereof.

10677. The method of item 10589 wherein the agent is ABT-578 or an analogue or derivative thereof.

10678. The method of item 10589 wherein the agent is an inosine monophosphate dehydrogenase (IMPDH) inhibitor.

10679. The method of item 10589 wherein the agent is an IMPDH inhibitor, wherein the IMPDH inhibitor is mycophenolic acid or an analogue or derivative thereof.

10680. The method of item 10589 wherein the agent is an IMPDH inhibitor, wherein the IMPDH inhibitor is 1-alpha-25 dihydroxy vitamin D₃ or an analogue or derivative thereof.

10681. The method of item 10589 wherein the agent is a leukotriene inhibitor.

10682. The method of item 10589 wherein the agent is a MCP-1 antagonist.

10683. The method of item 10589 wherein the agent is a MMP inhibitor.

10684. The method of item 10589 wherein the agent is an NF kappa B inhibitor.

10685. The method of item 10589 wherein the agent is an NF kappa B inhibitor, wherein the NF kappa B inhibitor is Bay 11-7082.

10686. The method of item 10589 wherein the agent is an NO agonist.

10687. The method of item 10589 wherein the agent is a p38 MAP kinase inhibitor.

10688. The method of item 10589 wherein the agent is a p38 MAP kinase inhibitor, wherein the p38 MAP kinase inhibitor is SB 202190.

10689. The method of item 10589 wherein the agent is a phosphodiesterase inhibitor.

10690. The method of item 10589 wherein the agent is a TGF beta inhibitor.

10691. The method of item 10589 wherein the agent is a thromboxane A2 antagonist.

10692. The method of item 10589 wherein the agent is a TNFa antagonist.

10693. The method of item 10589 wherein the agent is a TACE inhibitor.

10694. The method of item 10589 wherein the agent is a tyrosine kinase inhibitor.

10695. The method of item 10589 wherein the agent is a vitronectin inhibitor.

10696. The method of item 10589 wherein the agent is a fibroblast growth factor inhibitor.

10697. The method of item 10589 wherein the agent is a protein kinase inhibitor.

10698. The method of item 10589 wherein the agent is a PDGF receptor kinase inhibitor.

10699. The method of item 10589 wherein the agent is an endothelial growth factor receptor kinase inhibitor.

10700. The method of item 10589 wherein the agent is a retinoic acid receptor antagonist.

10701. The method of item 10589 wherein the agent is a platelet derived growth factor receptor kinase inhibitor.

10702. The method of item 10589 wherein the agent is a fibronogin antagonist.

10703. The method of item 10589 wherein the agent is an antimycotic agent.

10704. The method of item 10589 wherein the agent is an antimycotic agent, wherein the antimycotic agent is sulconizole.

10705. The method of item 10589 wherein the agent is a bisphosphonate.

10706. The method of item 10589 wherein the agent is a phospholipase A1 inhibitor.

10707. The method of item 10589 wherein the agent is a histamine H1/H2/H3 receptor antagonist.

10708. The method of item 10589 wherein the agent is a macrolide antibiotic.

10709. The method of item 10589 wherein the agent is a GPIIb/IIIa receptor antagonist.

10710. The method of item 10589 wherein the agent is an endothelin receptor antagonist.

10711. The method of item 10589 wherein the agent is a peroxisome proliferator-activated receptor agonist.

10712. The method of item 10589 wherein the agent is an estrogen receptor agent.

10713. The method of item 10589 wherein the agent is a somastostatin analogue.

10714. The method of item 10589 wherein the agent is a neurokinin 1 antagonist.

10715. The method of item 10589 wherein the agent is a neurokinin 3 antagonist.

10716. The method of item 10589 wherein the agent is a VLA-4 antagonist.

10717. The method of item 10589 wherein the agent is an osteoclast inhibitor.

10718. The method of item 10589 wherein the agent is a DNA topoisomerase ATP hydrolyzing inhibitor.

10719. The method of item 10589 wherein the agent is an angiotensin I converting enzyme inhibitor.

10720. The method of item 10589 wherein the agent is an angiotensin II antagonist.

10721. The method of item 10589 wherein the agent is an enkephalinase inhibitor.

10722. The method of item 10589 wherein the agent is a peroxisome proliferator-activated receptor gamma agonist insulin sensitizer.

10723. The method of item 10589 wherein the agent is a protein kinase C inhibitor.

10724. The method of item 10589 wherein the agent is a ROCK (rho-associated kinase) inhibitor.

10725. The method of item 10589 wherein the agent is a CXCR3 inhibitor.

10726. The method of item 10589 wherein the agent is an Itk inhibitor.

10727. The method of item 10589 wherein the agent is a cytosolic phospholipase A₂-alpha inhibitor.

10728. The method of item 10589 wherein the agent is a PPAR agonist.

10729. The method of item 10589 wherein the agent is an immunosuppressant.

10730. The method of item 10589 wherein the agent is an Erb inhibitor.

10731. The method of item 10589 wherein the agent is an apoptosis agonist.

10732. The method of item 10589 wherein the agent is a lipocortin agonist.

10733. The method of item 10589 wherein the agent is a VCAM-1 antagonist.

10734. The method of item 10589 wherein the agent is a collagen antagonist.

10735. The method of item 10589 wherein the agent is an alpha 2 integrin antagonist.

10736. The method of item 10589 wherein the agent is a TNF alpha inhibitor.

10737. The method of item 10589 wherein the agent is a nitric oxide inhibitor.

10738. The method of item 10589 wherein the agent is a cathepsin inhibitor.

10739. The method of item 10589 wherein the agent is not an anti-inflammatory agent.

10740. The method of item 10589 wherein the agent is not a steroid.

10741. The method of item 10589 wherein the agent is not a glucocorticosteroid.

10742. The method of item 10589 wherein the agent is not dexamethasone.

10743. The method of item 10589 wherein the agent is not an anti-infective agent.

10744. The method of item 10589 wherein the agent is not an antibiotic.

10745. The method of item 10589 wherein the agent is not an anti-fungal agent.

10746. The method of item 10589, wherein the composition comprises a polymer.

10747. The method of item 10589, wherein the composition comprises a polymeric carrier.

10748. The method of item 10589 wherein the anti-scarring agent inhibits adhesion between the device and a host into which the device is implanted.

10749. The method of item 10589 wherein the device delivers the anti-scarring agent locally to tissue proximate to the device.

10750. The method of item 10589 wherein the device has a coating that comprises the anti-scarring agent.

10751. The method of item 10589, wherein the device has a coating that comprises the agent and is disposed on a surface of the implant.

10752. The method of item 10589, wherein the device has a coating that comprises the agent and directly contacts the implant.

10753. The method of item 10589, wherein the device has a coating that comprises the agent and indirectly contacts the implant.

10754. The method of item 10589, wherein the device has a coating that comprises the agent and partially covers the implant.

10755. The method of item 10589, wherein the device has a coating that comprises the agent and completely covers the implant.

10756. The method of item 10589, wherein the device has a uniform coating.

10757. The method of item 10589, wherein the device has a non-uniform coating.

10758. The method of item 10589, wherein the device has a discontinuous coating.

10759. The method of item 10589, wherein the device has a patterned coating.

10760. The method of item 10589, wherein the device has a coating with a thickness of 100 μm or less.

10761. The method of item 10589, wherein the device has a coating with a thickness of 10 μm or less.

10762. The method of item 10589, wherein the device has a coating, and the coating adheres to the surface of the implant upon deployment of the implant.

10763. The method of item 10589, wherein the device has a coating, and wherein the coating is stable at room temperature for a period of 1 year.

10764. The method of item 10589, wherein the device has a coating, and wherein the anti-scarring agent is present in the coating in an amount ranging between about 0.0001% to about 1% by weight.

10765. The method of item 10589, wherein the device has a coating, and wherein the anti-scarring agent is present in the coating in an amount ranging between about 1% to about 10% by weight.

10766. The method of item 10589, wherein the device has a coating, and wherein the anti-scarring agent is present in the coating in an amount ranging between about 10% to about 25% by weight.

10767. The method of item 10589, wherein the device has a coating, and wherein the anti-scarring agent is present in the coating in an amount ranging between about 25% to about 70% by weight.

10768. The method of item 10589, wherein the device has a coating, and wherein the coating further comprises a polymer.

10769. The method of item 10589, wherein the device has a first coating having a first composition and a second coating having a second composition.

10770. The method of item 10589, wherein the device has a first coating having a first composition and a second coating having a second composition, wherein the first composition and the second composition are different.

10771. The method of item 10589, wherein the composition comprises a polymer.

10772. The method of item 10589, wherein the composition comprises a polymeric carrier.

10773. The method of item 10589, wherein the composition comprises a polymeric carrier, and wherein the polymeric carrier comprises a copolymer.

10774. The method of item 10589, wherein the composition comprises a polymeric carrier, and wherein the polymeric carrier comprises a block copolymer.

10775. The method of item 10589, wherein the composition comprises a polymeric carrier, and wherein the polymeric carrier comprises a random copolymer.

10776. The method of item 10589, wherein the composition comprises a polymeric carrier, and wherein the polymeric carrier comprises a biodegradable polymer.

10777. The method of item 10589, wherein the composition comprises a polymeric carrier, and wherein the polymeric carrier comprises a non-biodegradable polymer.

10778. The method of item 10589, wherein the composition comprises a polymeric carrier, and wherein the polymeric carrier comprises a hydrophilic polymer.

10779. The method of item 10589, wherein the composition comprises a polymeric carrier, and wherein the polymeric carrier comprises a hydrophobic polymer.

10780. The method of item 10589, wherein the composition comprises a polymeric carrier, and wherein the polymeric carrier comprises a polymer having hydrophilic domains.

10781. The method of item 10589, wherein the composition comprises a polymeric carrier, and wherein the polymeric carrier comprises a polymer having hydrophobic domains.

10782. The method of item 10589, wherein the composition comprises a polymeric carrier, and wherein the polymeric carrier comprises a non-conductive polymer.

10783. The method of item 10589, wherein the composition comprises a polymeric carrier, and wherein the polymeric carrier comprises an elastomer.

10784. The method of item 10589, wherein the composition comprises a polymeric carrier, and wherein the polymeric carrier comprises a hydrogel.

10785. The method of item 10589, wherein the composition comprises a polymeric carrier, and wherein the polymeric carrier comprises a silicone polymer.

10786. The method of item 10589, wherein the composition comprises a polymeric carrier, and wherein the polymeric carrier comprises a hydrocarbon polymer.

10787. The method of item 10589, wherein the composition comprises a polymeric carrier, and wherein the polymeric carrier comprises a styrene-derived polymer.

10788. The method of item 10589, wherein the composition comprises a polymeric carrier, and wherein the polymeric carrier comprises a butadiene polymer.

10789. The method of item 10589, wherein the composition comprises a polymeric carrier, and wherein the polymeric carrier comprises a macromer.

10790. The method of item 10589, wherein the composition comprises a polymeric carrier, and wherein the polymeric carrier comprises a poly(ethylene glycol)polymer.

10791. The method of item 10589 wherein the composition comprises a polymeric carrier, and wherein the polymeric carrier comprises an amorphous polymer.

10792. The method of item 10589, wherein the device comprises a lubricious coating.

10793. The method of item 10589 wherein the anti-scarring agent is located within pores or holes of the device.

10794. The method of item 10589 wherein the anti-scarring agent is located within a channel, lumen, or divet of the device.

10795. The method of item 10589, wherein the device comprises a second pharmaceutically active agent.

10796. The method of item 10589 wherein the device comprises an anti-inflammatory agent.

10797. The method of item 10589 wherein the device comprises an agent that inhibits infection.

10798. The method of item 10589 wherein the device comprises an agent that inhibits infection, and wherein the agent is an anthracycline.

10799. The method of item 10589 wherein the device comprises an agent that inhibits infection, and wherein the agent is doxorubicin.

10800. The method of item 10589 wherein the device comprises an agent that inhibits infection, and wherein the agent is mitoxantrone.

10801. The method of item 10589 wherein the device comprises an agent that inhibits infection, and wherein the agent is a fluoropyrimidine.

10802. The method of item 10589 wherein the device comprises an agent that inhibits infection, and wherein the agent is 5-fluorouracil (5-FU).

10803. The method of item 10589 wherein the device comprises an agent that inhibits infection, and wherein the agent is a folic acid antagonist.

10804. The method of item 10589 wherein the device comprises an agent that inhibits infection, and wherein the agent is methotrexate.

10805. The method of item 10589 wherein the device comprises an agent that inhibits infection, and wherein the agent is a podophylotoxin.

10806. The method of item 10589 wherein the device comprises an agent that inhibits infection, and wherein the agent is etoposide.

10807. The method of item 10589 wherein the device comprises an agent that inhibits infection, and wherein the agent is a camptothecin.

10808. The method of item 10589 wherein the device comprises an agent that inhibits infection, and wherein the agent is a hydroxyurea.

10809. The method of item 10589 wherein the device comprises an agent that inhibits infection, and wherein the agent is a platinum complex.

10810. The method of item 10589 wherein the device comprises an agent that inhibits infection, and wherein the agent is cisplatin.

10811. The method of item 10589, further comprising an anti-thrombotic agent.

10812. The method of item 10589 wherein the device comprises a visualization agent.

10813. The method of item 10589 wherein the device comprises a visualization agent, wherein the visualization agent is a radiopaque material, and wherein the radiopaque material comprises a metal, a halogenated compound, or a barium containing compound.

10814. The method of item 10589 wherein the device comprises a visualization agent, wherein the visualization agent is a radiopaque material, and wherein the radiopaque material comprises barium, tantalum, or technetium.

10815. The method of item 10589 wherein the device comprises a visualization agent, and wherein the visualization agent is a MRI responsive material.

10816. The method of item 10589 wherein the device comprises a visualization agent, and wherein the visualization agent comprises a gadolinium chelate.

10817. The method of item 10589 wherein the device comprises a visualization agent, and wherein the visualization agent comprises iron, magnesium, manganese, copper, or chromium.

10818. The method of item 10589 wherein the device comprises a visualization agent, and wherein the visualization agent comprises an iron oxide compound.

10819. The method of item 10589 wherein the device comprises a visualization agent, and wherein the visualization agent comprises a dye, pigment, or colorant.

10820. The method of item 10589 wherein the device comprises an echogenic material.

10821. The method of item 10589 wherein the device comprises an echogenic material, and wherein the echogenic material is in the form of a coating.

10822. The method of item 10589 wherein the device is sterile.

10823. The method of item 10589 wherein the anti-scarring agent is released into tissue in the vicinity of the device after deployment of the device.

10824. The method of item 10589 wherein the anti-scarring agent is released into tissue in the vicinity of the device after deployment of the device, and wherein the tissue is connective tissue.

10825. The method of item 10589 wherein the anti-scarring agent is released into tissue in the vicinity of the device after deployment of the device, and wherein the tissue is muscle tissue.

10826. The method of item 10589 wherein the anti-scarring agent is released into tissue in the vicinity of the device after deployment of the device, and wherein the tissue is nerve tissue.

10827. The method of item 10589 wherein the anti-scarring agent is released into tissue in the vicinity of the device after deployment of the device, and wherein the tissue is epithelium tissue.

10828. The method of item 10589 wherein the anti-scarring agent is released in effective concentrations from the device over a period ranging from the time of deployment of the device to about 1 year.

10829. The method of item 10589 wherein the anti-scarring agent is released in effective concentrations from the device over a period ranging from about 1 month to 6 months.

10830. The method of item 10589 wherein the anti-scarring agent is released in effective concentrations from the device over a period ranging from about 1-90 days.

10831. The method of item 10589 wherein the anti-scarring agent is released in effective concentrations from the device at a constant rate.

10832. The method of item 10589 wherein the anti-scarring agent is released in effective concentrations from the device at an increasing rate.

10833. The method of item 10589 wherein the anti-scarring agent is released in effective concentrations from the device at a decreasing rate.

10834. The method of item 10589 wherein the anti-scarring agent is released in effective concentrations from the composition comprising the anti-scarring agent by diffusion over a period ranging from the time of deployment of the device to about 90 days.

10835. The method of item 10589 wherein the anti-scarring agent is released in effective concentrations from the composition comprising the anti-scarring agent by erosion of the composition over a period ranging from the time of deployment of the device to about 90 days.

10836. The method of item 10589 wherein the device comprises about 0.01 μg to about 10 μg of the anti-scarring agent.

10837. The method of item 10589 wherein the device comprises about 10 μg to about 10 mg of the anti-scarring agent.

10838. The method of item 10589 wherein the device comprises about 10 mg to about 250 mg of the anti-scarring agent.

10839. The method of item 10589 wherein the device comprises about 250 mg to about 1000 mg of the anti-scarring agent.

10840. The method of item 10589 wherein the device comprises about 1000 mg to about 2500 mg of the anti-scarring agent.

10841. The method of item 10589 wherein a surface of the device comprises less than 0.01 μg of the anti-scarring agent per mm² of device surface to which the anti-scarring agent is applied.

10842. The method of item 10589 wherein a surface of the device comprises about 0.01 μg to about 1 μg of the anti-scarring agent per mm² of device surface to which the anti-scarring agent is applied.

10843. The method of item 10589 wherein a surface of the device comprises about 1 μg to about 10 μg of the anti-scarring agent per mm² of device surface to which the anti-scarring agent is applied.

10844. The method of item 10589 wherein a surface of the device comprises about 10 μg to about 250 μg of the anti-scarring agent per mm² of device surface to which the anti-scarring agent is applied.

10845. The method of item 10589 wherein a surface of the device comprises about 250 μg to about 1000 μg of the anti-scarring agent of anti-scarring agent per mm² of device surface to which the anti-scarring agent is applied.

10846. The method of item 10589 wherein a surface of the device comprises about 1000 μg to about 2500 μg of the anti-scarring agent per mm² of device surface to which the anti-scarring agent is applied.

10847. The method of item 10589 wherein the combining is performed by direct affixing the agent or the composition to the implant.

10848. The method of item 10589 wherein the combining is performed by spraying the agent or the component onto the implant.

10849. The method of item 10589 wherein the combining is performed by electrospraying the agent or the composition onto the implant.

10850. The method of item 10589 wherein the combining is performed by dipping the implant into a solution comprising the agent or the composition.

10851. The method of item 10589 wherein the combining is performed by covalently attaching the agent or the composition to the implant.

10852. The method of item 10589 wherein the combining is performed by non-covalently attaching the agent or the composition to the implant.

10853. The method of item 10589 wherein the combining is performed by coating the implant with a substance that contains the agent or the composition.

10854. The method of item 10589 wherein the combining is performed by coating the implant with a substance that absorbs the agent.

10855. The method of item 10589 wherein the combining is performed by interweaving a thread composed of, or coated with, the agent or the composition.

10856. The method of item 10589 wherein the combining is performed by covering all the implant with a sleeve that contains the agent or the composition.

10857. The method of item 10589 wherein the combining is performed by covering a portion of the implant with a sleeve that contains the agent or the composition.

10858. The method of item 10589 wherein the combining is performed by covering all the implant with a cover that contains the agent or the composition.

10859. The method of item 10589 wherein the combining is performed by covering a portion of the implant with a cover that contains the agent or the composition.

10860. The method of item 10589 wherein the combining is performed by covering all the implant with an electrospun fabric that contains the agent or the composition.

10861. The method of item 10589 wherein the combining is performed by covering a portion of the implant with an electrospun fabric that contains the agent or the composition.

10862. The method of item 10589 wherein the combining is performed by covering all the implant with a mesh that contains the agent or the composition.

10863. The method of item 10589 wherein the combining is performed by covering a portion of the implant with a mesh that contains the agent or the composition.

10864. The method of item 10589 wherein the combining is performed by constructing all the implant with the agent or the composition.

10865. The method of item 10589 wherein the combining is performed by constructing a portion of the implant with the agent or the composition.

10866. The method of item 10589 wherein the combining is performed by impregnating the implant with the agent or the composition.

10867. The method of item 10589 wherein the combining is performed by constructing all of the implant from a degradable polymer that releases the agent.

10868. The method of item 10589 wherein the combining is performed by constructing a portion of the implant from a degradable polymer that releases the agent.

10869. The method of item 10589 wherein the combining is performed by dipping the implant into a solution that comprise the agent and an inert solvent for the implant.

10870. The method of item 10589 wherein the combining is performed by dipping the implant into a solution that comprises the agent and a solvent that will swill the implant.

10871. The method of item 10589 wherein the combining is performed by dipping the implant into a solution that comprises the agent and a solvent that will dissolve the implant.

10872. The method of item 10589 wherein the combining is performed by dipping the implant into a solution that comprises the agent, a polymer and an inert solvent for the implant.

10873. The method of item 10589 wherein the combining is performed by dipping the implant into a solution that comprises the agent, a polymer and a solvent that will swill the implant.

10874. The method of item 10589 wherein the combining is performed by dipping the implant into a solution that comprises the agent, a polymer and a solvent that will dissolve the implant.

10875. The method of item 10589 wherein the combining is performed by spraying the implant into a solution that comprises the agent and an inert solvent for the implant.

10876. The method of item 10589 wherein the combining is performed by spraying the implant into a solution that comprises the agent and a solvent that will swill the implant.

10877. The method of item 10589 wherein the combining is performed by spraying the implant into a solution that comprises the agent and a solvent that will dissolve the implant.

10878. The method of item 10589 wherein the combining is performed by spraying the implant into a solution that comprises the agent, a polymer and an inert solvent for the implant.

10879. The method of item 10589 wherein the combining is performed by spraying the implant into a solution that comprises the agent, a polymer and a solvent that will swill the implant.

10880. The method of item 10589 wherein the combining is performed by spraying the implant into a solution that comprises the agent, a polymer and a solvent that will dissolve the implant.

10881. The method of item 10589 wherein the implant is a stent.

10882. The method of item 10589 wherein the implant is a coronary stent.

10883. The method of item 10589 wherein the implant is a peripheral stent.

10884. The method of item 10589 wherein the implant is a covered stent.

10885. The method of item 10589 wherein the implant is an intravascular catheter.

10886. The method of item 10589 wherein the implant is a microinjector catheter.

10887. The method of item 10589 wherein the implant is a drug delivery balloon.

10888. The method of item 10589 wherein the implant is a sweaty balloon.

10889. The method of item 10589 wherein the implant is a channel balloon.

10890. The method of item 10589 wherein the implant is a microinjector balloon.

10891. The method of item 10589 wherein the implant is a double balloon.

10892. The method of item 10589 wherein the implant is a spiral balloon.

10893. The method of item 10589 wherein the implant is a BHP balloon.

10894. The method of item 10589 wherein the implant is a transurethral needle ablation (TUNA) balloon.

10895. The method of item 10589 wherein the implant is a radio frequency needle ablation (RFNA) balloon.

10896. The method of item 10589 wherein the implant is a coronary drug infuction guidewire.

10897. A method of making a medical device comprising: combining a vascular graft or wrap implant and an anti-scarring agent or a composition comprising an anti-scarring agent, wherein the agent inhibits scarring between the device and a host into which the device is implanted.

10898. The method of item 10897 wherein the agent inhibits cell regeneration.

10899. The method of item 10897 wherein the agent inhibits angiogenesis.

10900. The method of item 10897 wherein the agent inhibits fibroblast migration.

10901. The method of item 10897 wherein the agent inhibits fibroblast proliferation.

10902. The method of item 10897 wherein the agent inhibits deposition of extracellular matrix.

10903. The method of item 10897 wherein the agent inhibits tissue remodeling.

10904. The method of item 10897 wherein the agent is an angiogenesis inhibitor.

10905. The method of item 10897 wherein the agent is a 5-lipoxygenase inhibitor or antagonist.

10906. The method of item 10897 wherein the agent is a chemokine receptor antagonist.

10907. The method of item 10897 wherein the agent is a cell cycle inhibitor.

10908. The method of item 10897 wherein the agent is a taxane.

10909. The method of item 10897 wherein the agent is an anti-microtubule agent.

10910. The method of item 10897 wherein the agent is paclitaxel.

10911. The method of item 10897 wherein the agent is not paclitaxel.

10912. The method of item 10897 wherein the agent is an analogue or derivative of paclitaxel.

10913. The method of item 10897 wherein the agent is a vinca alkaloid.

10914. The method of item 10897 wherein the agent is camptothecin or an analogue or derivative thereof.

10915. The method of item 10897 wherein the agent is a podophyllotoxin.

10916. The method of item 10897 wherein the agent is a podophyllotoxin, wherein the podophyllotoxin is etoposide or an analogue or derivative thereof.

10917. The method of item 10897 wherein the agent is an anthracycline.

10918. The method of item 10897 wherein the agent is an anthracycline, wherein the anthracycline is doxorubicin or an analogue or derivative thereof.

10919. The method of item 10897 wherein the agent is an anthracycline, wherein the anthracycline is mitoxantrone or an analogue or derivative thereof.

10920. The method of item 10897 wherein the agent is a platinum compound.

10921. The method of item 10897 wherein the agent is a nitrosourea.

10922. The method of item 10897 wherein the agent is a nitroimidazole.

10923. The method of item 10897 wherein the agent is a folic acid antagonist.

10924. The method of item 10897 wherein the agent is a cytidine analogue.

10925. The method of item 10897 wherein the agent is a pyrimidine analogue.

10926. The method of item 10897 wherein the agent is a fluoropyrimidine analogue.

10927. The method of item 10897 wherein the agent is a purine analogue.

10928. The method of item 10897 wherein the agent is a nitrogen mustard or an analogue or derivative thereof.

10929. The method of item 10897 wherein the agent is a hydroxyurea.

10930. The method of item 10897 wherein the agent is a mytomicin or an analogue or derivative thereof.

10931. The method of item 10897 wherein the agent is an alkyl sulfonate.

10932. The method of item 10897 wherein the agent is a benzamide or an analogue or derivative thereof.

10933. The method of item 10897 wherein the agent is a nicotinamide or an analogue or derivative thereof.

10934. The method of item 10897 wherein the agent is a halogenated sugar or an analogue or derivative thereof.

10935. The method of item 10897 wherein the agent is a DNA alkylating agent.

10936. The method of item 10897 wherein the agent is an anti-microtubule agent.

10937. The method of item 10897 wherein the agent is a topoisomerase inhibitor.

10938. The method of item 10897 wherein the agent is a DNA cleaving agent.

10939. The method of item 10897 wherein the agent is an antimetabolite.

10940. The method of item 10897 wherein the agent inhibits adenosine deaminase.

10941. The method of item 10897 wherein the agent inhibits purine ring synthesis.

10942. The method of item 10897 wherein the agent is a nucleotide interconversion inhibitor.

10943. The method of item 10897 wherein the agent inhibits dihydrofolate reduction.

10944. The method of item 10897 wherein the agent blocks thymidine monophosphate.

10945. The method of item 10897 wherein the agent causes DNA damage.

10946. The method of item 10897 wherein the agent is a DNA intercalation agent.

10947. The method of item 10897 wherein the agent is a RNA synthesis inhibitor.

10948. The method of item 10897 wherein the agent is a pyrimidine synthesis inhibitor.

10949. The method of item 10897 wherein the agent inhibits ribonucleotide synthesis or function.

10950. The method of item 10897 wherein the agent inhibits thymidine monophosphate synthesis or function.

10951. The method of item 10897 wherein the agent inhibits DNA synthesis.

10952. The method of item 10897 wherein the agent causes DNA adduct formation.

10953. The method of item 10897 wherein the agent inhibits protein synthesis.

10954. The method of item 10897 wherein the agent inhibits microtubule function.

10955. The method of item 10897 wherein the agent is a cyclin dependent protein kinase inhibitor.

10956. The method of item 10897 wherein the agent is an epidermal growth factor kinase inhibitor.

10957. The method of item 10897 wherein the agent is an elastase inhibitor.

10958. The method of item 10897 wherein the agent is a factor Xa inhibitor.

10959. The method of item 10897 wherein the agent is a farnesyltransferase inhibitor.

10960. The method of item 10897 wherein the agent is a fibrinogen antagonist.

10961. The method of item 10897 wherein the agent is a guanylate cyclase stimulant.

10962. The method of item 10897 wherein the agent is a heat shock protein 90 antagonist.

10963. The method of item 10897 wherein the agent is a heat shock protein 90 antagonist, wherein the heat shock protein 90 antagonist is geldanamycin or an analogue or derivative thereof.

10964. The method of item 10897 wherein the agent is a guanylate cyclase stimulant.

10965. The method of item 10897 wherein the agent is a HMGCoA reductase inhibitor.

10966. The method of item 10897 wherein the agent is a HMGCoA reductase inhibitor, wherein the HMGCoA reductase inhibitor is simvastatin or an analogue or derivative thereof.

10967. The method of item 10897 wherein the agent is a hydroorotate dehydrogenase inhibitor.

10968. The method of item 10897 wherein the agent is an IKK2 inhibitor.

10969. The method of item 10897 wherein the agent is an IL-1 antagonist.

10970. The method of item 10897 wherein the agent is an ICE antagonist.

10971. The method of item 10897 wherein the agent is an IRAK antagonist.

10972. The method of item 10897 wherein the agent is an IL-4 agonist.

10973. The method of item 10897 wherein the agent is an immunomodulatory agent.

10974. The method of item 10897 wherein the agent is sirolimus or an analogue or derivative thereof.

10975. The method of item 10897 wherein the agent is not sirolimus.

10976. The method of item 10897 wherein the agent is everolimus or an analogue or derivative thereof.

10977. The method of item 10897 wherein the agent is tacrolimus or an analogue or derivative thereof.

10978. The method of item. 10897 wherein the agent is not tacrolimus.

10979. The method of item 10897 wherein the agent is biolmus or an analogue or derivative thereof.

10980. The method of item 10897 wherein the agent is tresperimus or an analogue or derivative thereof.

10981. The method of item 10897 wherein the agent is auranofin or an analogue or derivative thereof.

10982. The method of item 10897 wherein the agent is 27-0-demethylrapamycin or an analogue or derivative thereof.

10983. The method of item 10897 wherein the agent is gusperimus or an analogue or derivative thereof.

10984. The method of item 10897 wherein the agent is pimecrolimus or an analogue or derivative thereof.

10985. The method of item 10897 wherein the agent is ABT-578 or an analogue or derivative thereof.

10986. The method of item 10897 wherein the agent is an inosine monophosphate dehydrogenase (IMPDH) inhibitor.

10987. The method of item 10897 wherein the agent is an IMPDH inhibitor, wherein the IMPDH inhibitor is mycophenolic acid or an analogue or derivative thereof.

10988. The method of item 10897 wherein the agent is an IMPDH inhibitor, wherein the IMPDH inhibitor is 1-alpha-25 dihydroxy vitamin D₃ or an analogue or derivative thereof.

10989. The method of item 10897 wherein the agent is a leukotriene inhibitor.

10990. The method of item 10897 wherein the agent is a MCP-1 antagonist.

10991. The method of item 10897 wherein the agent is a MMP inhibitor.

10992. The method of item 10897 wherein the agent is an NF kappa B inhibitor.

10993. The method of item 10897 wherein the agent is an NF kappa B inhibitor, wherein the NF kappa B inhibitor is Bay 11-7082.

10994. The method of item 10897 wherein the agent is an NO agonist.

10995. The method of item 10897 wherein the agent is a p38 MAP kinase inhibitor.

10996. The method of item 10897 wherein the agent is a p38 MAP kinase inhibitor, wherein the p38 MAP kinase inhibitor is SB 202190.

10997. The method of item 10897 wherein the agent is a phosphodiesterase inhibitor.

10998. The method of item 10897 wherein the agent is a TGF beta inhibitor.

10999. The method of item 10897 wherein the agent is a thromboxane A2 antagonist.

11000. The method of item 10897 wherein the agent is a TNFa antagonist.

11001. The method of item 10897 wherein the agent is a TACE inhibitor.

11002. The method of item 10897 wherein the agent is a tyrosine kinase inhibitor.

11003. The method of item 10897 wherein the agent is a vitronectin inhibitor.

11004. The method of item 10897 wherein the agent is a fibroblast growth factor inhibitor.

11005. The method of item 10897 wherein the agent is a protein kinase inhibitor.

11006. The method of item 10897 wherein the agent is a PDGF receptor kinase inhibitor.

11007. The method of item 10897 wherein the agent is an endothelial growth factor receptor kinase inhibitor.

11008. The method of item 10897 wherein the agent is a retinoic acid receptor antagonist.

11009. The method of item 10897 wherein the agent is a platelet derived growth factor receptor kinase inhibitor.

11010. The method of item 10897 wherein the agent is a fibronogin antagonist.

11011. The method of item 10897 wherein the agent is an antimycotic agent.

11012. The method of item 10897 wherein the agent is an antimycotic agent, wherein the antimycotic agent is sulconizole.

11013. The method of item 10897 wherein the agent is a bisphosphonate.

11014. The method of item 10897 wherein the agent is a phospholipase A1 inhibitor.

11015. The method of item 10897 wherein the agent is a histamine H1/H2/H3 receptor antagonist.

11016. The method of item 10897 wherein the agent is a macrolide antibiotic.

11017. The method of item 10897 wherein the agent is a GPIIb/IIIa receptor antagonist.

11018. The method of item 10897 wherein the agent is an endothelin receptor antagonist.

11019. The method of item 10897 wherein the agent is a peroxisome proliferator-activated receptor agonist.

11020. The method of item 10897 wherein the agent is an estrogen receptor agent.

11021. The method of item 10897 wherein the agent is a somastostatin analogue.

11022. The method of item 10897 wherein the agent is a neurokinin 1 antagonist.

11023. The method of item 10897 wherein the agent is a neurokinin 3 antagonist.

11024. The method of item 10897 wherein the agent is a VLA-4 antagonist.

11025. The method of item 10897 wherein the agent is an osteoclast inhibitor.

11026. The method of item 10897 wherein the agent is a DNA topoisomerase ATP hydrolyzing inhibitor.

11027. The method of item 10897 wherein the agent is an angiotensin I converting enzyme inhibitor.

11028. The method of item 10897 wherein the agent is an angiotensin II antagonist.

11029. The method of item 10897 wherein the agent is an enkephalinase inhibitor.

11030. The method of item 10897 wherein the agent is a peroxisome proliferator-activated receptor gamma agonist insulin sensitizer.

11031. The method of item 10897 wherein the agent is a protein kinase C inhibitor.

11032. The method of item 10897 wherein the agent is a ROCK (rho-associated kinase) inhibitor.

11033. The method of item 10897 wherein the agent is a CXCR3 inhibitor.

11034. The method of item 10897 wherein the agent is an Itk inhibitor.

11035. The method of item 10897 wherein the agent is a cytosolic phospholipase A₂-alpha inhibitor.

11036. The method of item 10897 wherein the agent is a PPAR agonist.

11037. The method of item 10897 wherein the agent is an immunosuppressant.

11038. The method of item 10897 wherein the agent is an Erb inhibitor.

11039. The method of item 10897 wherein the agent is an apoptosis agonist.

11040. The method of item 10897 wherein the agent is a lipocortin agonist.

11041. The method of item 10897 wherein the agent is a VCAM-1 antagonist.

11042. The method of item 10897 wherein the agent is a collagen antagonist.

11043. The method of item 10897 wherein the agent is an alpha 2 integrin antagonist.

11044. The method of item 10897 wherein the agent is a TNF alpha inhibitor.

11045. The method of item 10897 wherein the agent is a nitric oxide inhibitor.

11046. The method of item 10897 wherein the agent is a cathepsin inhibitor.

11047. The method of item 10897 wherein the agent is not an anti-inflammatory agent.

11048. The method of item 10897 wherein the agent is not a steroid.

11049. The method of item 10897 wherein the agent is not a glucocorticosteroid.

11050. The method of item 10897 wherein the agent is not dexamethasone.

11051. The method of item 10897 wherein the agent is not an anti-infective agent.

11052. The method of item 10897 wherein the agent is not an antibiotic.

11053. The method of item 10897 wherein the agent is not an anti-fungal agent.

11054. The method of item 10897, wherein the composition comprises a polymer.

11055. The method of item 10897, wherein the composition comprises a polymeric carrier.

11056. The method of item 10897 wherein the anti-scarring agent inhibits adhesion between the device and a host into which the device is implanted.

11057. The method of item 10897 wherein the device delivers the anti-scarring agent locally to tissue proximate to the device.

11058. The method of item 10897 wherein the device has a coating that comprises the anti-scarring agent.

11059. The method of item 10897, wherein the device has a coating that comprises the agent and is disposed on a surface of the implant.

11060. The method of item 10897, wherein the device has a coating that comprises the agent and directly contacts the implant.

11061. The method of item 10897, wherein the device has a coating that comprises the agent and indirectly contacts the implant.

11062. The method of item 10897, wherein the device has a coating that comprises the agent and partially covers the implant.

11063. The method of item 10897, wherein the device has a coating that comprises the agent and completely covers the implant.

11064. The method of item 10897, wherein the device has a uniform coating.

11065. The method of item 10897, wherein the device has a non-uniform coating.

11066. The method of item 10897, wherein the device has a discontinuous coating.

11067. The method of item 10897, wherein the device has a patterned coating.

11068. The method of item 10897, wherein the device has a coating with a thickness of 100 μm or less.

11069. The method of item 10897, wherein the device has a coating with a thickness of 10 μm or less.

11070. The method of item 10897, wherein the device has a coating, and the coating adheres to the surface of the implant upon deployment of the implant.

11071. The method of item 10897, wherein the device has a coating, and wherein the coating is stable at room temperature for a period of 1 year.

11072. The method of item 10897, wherein the device has a coating, and wherein the anti-scarring agent is present in the coating in an amount ranging between about 0.0001% to about 1% by weight.

11073. The method of item 10897, wherein the device has a coating, and wherein the anti-scarring agent is present in the coating in an amount ranging between about 1% to about 10% by weight.

11074. The method of item 10897, wherein the device has a coating, and wherein the anti-scarring agent is present in the coating in an amount ranging between about 10% to about 25% by weight.

11075. The method of item 10897, wherein the device has a coating, and wherein the anti-scarring agent is present in the coating in an amount ranging between about 25% to about 70% by weight.

11076. The method of item 10897, wherein the device has a coating, and wherein the coating further comprises a polymer.

11077. The method of item 10897, wherein the device has a first coating having a first composition and a second coating having a second composition.

11078. The method of item 10897, wherein the device has a first coating having a first composition and a second coating having a second composition, wherein the first composition and the second composition are different.

11079. The method of item 10897, wherein the composition comprises a polymer.

11080. The method of item 10897, wherein the composition comprises a polymeric carrier.

11081. The method of item 10897, wherein the composition comprises a polymeric carrier, and wherein the polymeric carrier comprises a copolymer.

11082. The method of item 10897, wherein the composition comprises a polymeric carrier, and wherein the polymeric carrier comprises a block copolymer.

11083. The method of item 10897, wherein the composition comprises a polymeric carrier, and wherein the polymeric carrier comprises a random copolymer.

11084. The method of item 10897, wherein the composition comprises a polymeric carrier, and wherein the polymeric carrier comprises a biodegradable polymer.

11085. The method of item 10897, wherein the composition comprises a polymeric carrier, and wherein the polymeric carrier comprises a non-biodegradable polymer.

11086. The method of item 10897, wherein the composition comprises a polymeric carrier, and wherein the polymeric carrier comprises a hydrophilic polymer.

11087. The method of item 10897, wherein the composition comprises a polymeric carrier, and wherein the polymeric carrier comprises a hydrophobic polymer.

11088. The method of item 10897, wherein the composition comprises a polymeric carrier, and wherein the polymeric carrier comprises a polymer having hydrophilic domains.

11089. The method of item 10897, wherein the composition comprises a polymeric carrier, and wherein the polymeric carrier comprises a polymer having hydrophobic domains.

11090. The method of item 10897, wherein the composition comprises a polymeric carrier, and wherein the polymeric carrier comprises a non-conductive polymer.

11091. The method of item 10897, wherein the composition comprises a polymeric carrier, and wherein the polymeric carrier comprises an elastomer.

11092. The method of item 10897, wherein the composition comprises a polymeric carrier, and wherein the polymeric carrier comprises a hydrogel.

11093. The method of item 10897, wherein the composition comprises a polymeric carrier, and wherein the polymeric carrier comprises a silicone polymer.

11094. The method of item 10897, wherein the composition comprises a polymeric carrier, and wherein the polymeric carrier comprises a hydrocarbon polymer.

11095. The method of item 10897, wherein the composition comprises a polymeric carrier, and wherein the polymeric carrier comprises a styrene-derived polymer.

11096. The method of item 10897, wherein the composition comprises a polymeric carrier, and wherein the polymeric carrier comprises a butadiene polymer.

11097. The method of item 10897, wherein the composition comprises a polymeric carrier, and wherein the polymeric carrier comprises a macromer.

11098. The method of item 10897, wherein the composition comprises a polymeric carrier, and wherein the polymeric carrier comprises a poly(ethylene glycol)polymer.

11099. The method of item 10897 wherein the composition comprises a polymeric carrier, and wherein the polymeric carrier comprises an amorphous polymer.

11100. The method of item 10897, wherein the device comprises a lubricious coating.

11101. The method of item 10897 wherein the anti-scarring agent is located within pores or holes of the device.

11102. The method of item 10897 wherein the anti-scarring agent is located within a channel, lumen, or divet of the device.

11103. The method of item 10897, wherein the device comprises a second pharmaceutically active agent.

11104. The method of item 10897 wherein the device comprises an anti-inflammatory agent.

11105. The method of item 10897 wherein the device comprises an agent that inhibits infection.

11106. The method of item 10897 wherein the device comprises an agent that inhibits infection, and wherein the agent is an anthracycline.

11107. The method of item 10897 wherein the device comprises an agent that inhibits infection, and wherein the agent is doxorubicin.

11108. The method of item 10897 wherein the device comprises an agent that inhibits infection, and wherein the agent is mitoxantrone.

11109. The method of item 10897 wherein the device comprises an agent that inhibits infection, and wherein the agent is a fluoropyrimidine.

11110. The method of item 10897 wherein the device comprises an agent that inhibits infection, and wherein the agent is 5-fluorouracil (5-FU).

11111. The method of item 10897 wherein the device comprises an agent that inhibits infection, and wherein the agent is a folic acid antagonist.

11112. The method of item 10897 wherein the device comprises an agent that inhibits infection, and wherein the agent is methotrexate.

11113. The method of item 10897 wherein the device comprises an agent that inhibits infection, and wherein the agent is a podophylotoxin.

11114. The method of item 10897 wherein the device comprises an agent that inhibits infection, and wherein the agent is etoposide.

11115. The method of item 10897 wherein the device comprises an agent that inhibits infection, and wherein the agent is a camptothecin.

11116. The method of item 10897 wherein the device comprises an agent that inhibits infection, and wherein the agent is a hydroxyurea.

11117. The method of item 10897 wherein the device comprises an agent that inhibits infection, and wherein the agent is a platinum complex.

11118. The method of item 10897 wherein the device comprises an agent that inhibits infection, and wherein the agent is cisplatin.

11119. The method of item 10897, further comprising an anti-thrombotic agent.

11120. The method of item 10897 wherein the device comprises a visualization agent.

11121. The method of item 10897 wherein the device comprises a visualization agent, wherein the visualization agent is a radiopaque material, and wherein the radiopaque material comprises a metal, a halogenated compound, or a barium containing compound.

11122. The method of item 10897 wherein the device comprises a visualization agent, wherein the visualization agent is a radiopaque material, and wherein the radiopaque material comprises barium, tantalum, or technetium.

11123. The method of item 10897 wherein the device comprises a visualization agent, and wherein the visualization agent is a MRI responsive material.

11124. The method of item 10897 wherein the device comprises a visualization agent, and wherein the visualization agent comprises a gadolinium chelate.

11125. The method of item 10897 wherein the device comprises a visualization agent, and wherein the visualization agent comprises iron, magnesium, manganese, copper, or chromium.

11126. The method of item 10897 wherein the device comprises a visualization agent, and wherein the visualization agent comprises an iron oxide compound.

11127. The method of item 10897 wherein the device comprises a visualization agent, and wherein the visualization agent comprises a dye, pigment, or colorant.

11128. The method of item 10897 wherein the device comprises an echogenic material.

11129. The method of item 10897 wherein the device comprises an echogenic material, and wherein the echogenic material is in the form of a coating.

11130. The method of item 10897 wherein the device is sterile.

11131. The method of item 10897 wherein the anti-scarring agent is released into tissue in the vicinity of the device after deployment of the device.

11132. The method of item 10897 wherein the anti-scarring agent is released into tissue in the vicinity of the device after deployment of the device, and wherein the tissue is connective tissue.

11133. The method of item 10897 wherein the anti-scarring agent is released into tissue in the vicinity of the device after deployment of the device, and wherein the tissue is muscle tissue.

11134. The method of item 10897 wherein the anti-scarring agent is released into tissue in the vicinity of the device after deployment of the device, and wherein the tissue is nerve tissue.

11135. The method of item 10897 wherein the anti-scarring agent is released into tissue in the vicinity of the device after deployment of the device, and wherein the tissue is epithelium tissue.

11136. The method of item 10897 wherein the anti-scarring agent is released in effective concentrations from the device over a period ranging from the time of deployment of the device to about 1 year.

11137. The method of item 10897 wherein the anti-scarring agent is released in effective concentrations from the device over a period ranging from about 1 month to 6 months.

11138. The method of item 10897 wherein the anti-scarring agent is released in effective concentrations from the device over a period ranging from about 1-90 days.

11139. The method of item 10897 wherein the anti-scarring agent is released in effective concentrations from the device at a constant rate.

11140. The method of item 10897 wherein the anti-scarring agent is released in effective concentrations from the device at an increasing rate.

11141. The method of item 10897 wherein the anti-scarring agent is released in effective concentrations from the device at a decreasing rate.

11142. The method of item 10897 wherein the anti-scarring agent is released in effective concentrations from the composition comprising the anti-scarring agent by diffusion over a period ranging from the time of deployment of the device to about 90 days.

11143. The method of item 10897 wherein the anti-scarring agent is released in effective concentrations from the composition comprising the anti-scarring agent by erosion of the composition over a period ranging from the time of deployment of the device to about 90 days.

11144. The method of item 10897 wherein the device comprises about 0.01 μg to about 10 μg of the anti-scarring agent.

11145. The method of item 10897 wherein the device comprises about 10 μg to about 10 mg of the anti-scarring agent.

11146. The method of item 10897 wherein the device comprises about 10 mg to about 250 mg of the anti-scarring agent.

11147. The method of item 10897 wherein the device comprises about 250 mg to about 1000 mg of the anti-scarring agent.

11148. The method of item 10897 wherein the device comprises about 1000 mg to about 2500 mg of the anti-scarring agent.

11149. The method of item 10897 wherein a surface of the device comprises less than 0.01 μg of the anti-scarring agent per mm² of device surface to which the anti-scarring agent is applied.

11150. The method of item 10897 wherein a surface of the device comprises about 0.01 μg to about 1 μg of the anti-scarring agent per mm² of device surface to which the anti-scarring agent is applied.

11151. The method of item 10897 wherein a surface of the device comprises about 1 μg to about 10 μg of the anti-scarring agent per mm² of device surface to which the anti-scarring agent is applied.

11152. The method of item 10897 wherein a surface of the device comprises about 10 μg to about 250 μg of the anti-scarring agent per mm² of device surface to which the anti-scarring agent is applied.

11153. The method of item 10897 wherein a surface of the device comprises about 250 μg to about 1000 μg of the anti-scarring agent of anti-scarring agent per mm² of device surface to which the anti-scarring agent is applied.

11154. The method of item 10897 wherein a surface of the device comprises about 1000 μg to about 2500 μg of the anti-scarring agent per mm² of device surface to which the anti-scarring agent is applied.

11155. The method of item 10897 wherein the combining is performed by direct affixing the agent or the composition to the implant.

11156. The method of item 10897 wherein the combining is performed by spraying the agent or the component onto the implant.

11157. The method of item 10897 wherein the combining is performed by electrospraying the agent or the composition onto the implant.

11158. The method of item 10897 wherein the combining is performed by dipping the implant into a solution comprising the agent or the composition.

11159. The method of item 10897 wherein the combining is performed by covalently attaching the agent or the composition to the implant.

11160. The method of item 10897 wherein the combining is performed by non-covalently attaching the agent or the composition to the implant.

11161. The method of item 10897 wherein the combining is performed by coating the implant with a substance that contains the agent or the composition.

11162. The method of item 10897 wherein the combining is performed by coating the implant with a substance that absorbs the agent.

11163. The method of item 10897 wherein the combining is performed by interweaving a thread composed of, or coated with, the agent or the composition.

11164. The method of item 10897 wherein the combining is performed by covering all the implant with a sleeve that contains the agent or the composition.

11165. The method of item 10897 wherein the combining is performed by covering a portion of the implant with a sleeve that contains the agent or the composition.

11166. The method of item 10897 wherein the combining is performed by covering all the implant with a cover that contains the agent or the composition.

11167. The method of item 10897 wherein the combining is performed by covering a portion of the implant with a cover that contains the agent or the composition.

11168. The method of item 10897 wherein the combining is performed by covering all the implant with an electrospun fabric that contains the agent or the composition.

11169. The method of item 10897 wherein the combining is performed by covering a portion of the implant with an electrospun fabric that contains the agent or the composition.

11170. The method of item 10897 wherein the combining is performed by covering all the implant with a mesh that contains the agent or the composition.

11171. The method of item 10897 wherein the combining is performed by covering a portion of the implant with a mesh that contains the agent or the composition.

11172. The method of item 10897 wherein the combining is performed by constructing all the implant with the agent or the composition.

11173. The method of item 10897 wherein the combining is performed by constructing a portion of the implant with the agent or the composition.

11174. The method of item 10897 wherein the combining is performed by impregnating the implant with the agent or the composition.

11175. The method of item 10897 wherein the combining is performed by constructing all of the implant from a degradable polymer that releases the agent.

11176. The method of item 10897 wherein the combining is performed by constructing a portion of the implant from a degradable polymer that releases the agent.

11177. The method of item 10897 wherein the combining is performed by dipping the implant into a solution that comprise the agent and an inert solvent for the implant.

11178. The method of item 10897 wherein the combining is performed by dipping the implant into a solution that comprises the agent and a solvent that will swill the implant.

11179. The method of item 10897 wherein the combining is performed by dipping the implant into a solution that comprises the agent and a solvent that will dissolve the implant.

11180. The method of item 10897 wherein the combining is performed by dipping the implant into a solution that comprises the agent, a polymer and an inert solvent for the implant.

11181. The method of item 10897 wherein the combining is performed by dipping the implant into a solution that comprises the agent, a polymer and a solvent that will swill the implant.

11182. The method of item 10897 wherein the combining is performed by dipping the implant into a solution that comprises the agent, a polymer and a solvent that will dissolve the implant.

11183. The method of item 10897 wherein the combining is performed by spraying the implant into a solution that comprises the agent and an inert solvent for the implant.

11184. The method of item 10897 wherein the combining is performed by spraying the implant into a solution that comprises the agent and a solvent that will swill the implant.

11185. The method of item 10897 wherein the combining is performed by spraying the implant into a solution that comprises the agent and a solvent that will dissolve the implant.

11186. The method of item 10897 wherein the combining is performed by spraying the implant into a solution that comprises the agent, a polymer and an inert solvent for the implant.

11187. The method of item 10897 wherein the combining is performed by spraying the implant into a solution that comprises the agent, a polymer and a solvent that will swill the implant.

11188. The method of item 10897 wherein the combining is performed by spraying the implant into a solution that comprises the agent, a polymer and a solvent that will dissolve the implant.

11189. The method of item 10897 wherein the implant is a synthetic bypass graft.

11190. The method of item 10897 wherein the implant is a femoral-popliteal synthetic bypass graft.

11191. The method of item 10897 wherein the implant is a femoral-femoral synthetic bypass graft.

11192. The method of item 10897 wherein the implant is a axillary-femoral synthetic bypass graft.

11193. The method of item 10897 wherein the implant is a vein graft.

11194. The method of item 10897 wherein the implant is a peripheral vein graft.

11195. The method of item 10897 wherein the implant is a coronary vein graft.

11196. The method of item 10897 wherein the implant is an internal mammary graft.

11197. The method of item 10897 wherein the implant is an internal mammary coronary graft.

11198. The method of item 10897 wherein the implant is a bifurcated vascular graft.

11199. The method of item 10897 wherein the implant is vascular wrap.

11200. A method of making a medical device comprising: combining an implant for hemodialysis access (i.e., a hemodialysis access device) and an anti-scarring agent or a composition comprising an anti-scarring agent, wherein the agent inhibits scarring between the device and a host into which the device is implanted.

11201. The method of item 11200 wherein the agent inhibits cell regeneration.

11202. The method of item 11200 wherein the agent inhibits angiogenesis.

11203. The method of item 11200 wherein the agent inhibits fibroblast migration.

11204. The method of item 11200 wherein the agent inhibits fibroblast proliferation.

11205. The method of item 11200 wherein the agent inhibits deposition of extracellular matrix.

11206. The method of item 11200 wherein the agent inhibits tissue remodeling.

11207. The method of item 11200 wherein the agent is an angiogenesis inhibitor.

11208. The method of item 11200 wherein the agent is a 5-lipoxygenase inhibitor or antagonist.

11209. The method of item 11200 wherein the agent is a chemokine receptor antagonist.

11210. The method of item 11200 wherein the agent is a cell cycle inhibitor.

11211. The method of item 11200 wherein the agent is a taxane.

11212. The method of item 11200 wherein the agent is an anti-microtubule agent.

11213. The method of item 11200 wherein the agent is paclitaxel.

11214. The method of item 11200 wherein the agent is not paclitaxel.

11215. The method of item 11200 wherein the agent is an analogue or derivative of paclitaxel.

11216. The method of item 11200 wherein the agent is a vinca alkaloid.

11217. The method of item 11200 wherein the agent is camptothecin or an analogue or derivative thereof.

11218. The method of item 11200 wherein the agent is a podophyllotoxin.

11219. The method of item 11200 wherein the agent is a podophyllotoxin, wherein the podophyllotoxin is etoposide or an analogue or derivative thereof.

11220. The method of item 11200 wherein the agent is an anthracycline.

11221. The method of item 11200 wherein the agent is an anthracycline, wherein the anthracycline is doxorubicin or an analogue or derivative thereof.

11222. The method of item 11200 wherein the agent is an anthracycline, wherein the anthracycline is mitoxantrone or an analogue or derivative thereof.

11223. The method of item 11200 wherein the agent is a platinum compound.

11224. The method of item 11200 wherein the agent is a nitrosourea.

11225. The method of item 11200 wherein the agent is a nitroimidazole.

11226. The method of item 11200 wherein the agent is a folic acid antagonist.

11227. The method of item 11200 wherein the agent is a cytidine analogue.

11228. The method of item 11200 wherein the agent is a pyrimidine analogue.

11229. The method of item 11200 wherein the agent is a fluoropyrimidine analogue.

11230. The method of item 11200 wherein the agent is a purine analogue.

11231. The method of item 11200 wherein the agent is a nitrogen mustard or an analogue or derivative thereof.

11232. The method of item 11200 wherein the agent is a hydroxyurea.

11233. The method of item 11200 wherein the agent is a mytomicin or an analogue or derivative thereof.

11234. The method of item 11200 wherein the agent is an alkyl sulfonate.

11235. The method of item 11200 wherein the agent is a benzamide or an analogue or derivative thereof.

11236. The method of item 11200 wherein the agent is a nicotinamide or an analogue or derivative thereof.

11237. The method of item 11200 wherein the agent is a halogenated sugar or an analogue or derivative thereof.

11238. The method of item 11200 wherein the agent is a DNA alkylating agent.

11239. The method of item 11200 wherein the agent is an anti-microtubule agent.

11240. The method of item 11200 wherein the agent is a topoisomerase inhibitor.

11241. The method of item 11200 wherein the agent is a DNA cleaving agent.

11242. The method of item 11200 wherein the agent is an antimetabolite.

11243. The method of item 11200 wherein the agent inhibits adenosine deaminase.

11244. The method of item 11200 wherein the agent inhibits purine ring synthesis.

11245. The method of item 11200 wherein the agent is a nucleotide interconversion inhibitor.

11246. The method of item 11200 wherein the agent inhibits dihydrofolate reduction.

11247. The method of item 11200 wherein the agent blocks thymidine monophosphate.

11248. The method of item 11200 wherein the agent causes DNA damage.

11249. The method of item 11200 wherein the agent is a DNA intercalation agent.

11250. The method of item 11200 wherein the agent is a RNA synthesis inhibitor.

11251. The method of item 11200 wherein the agent is a pyrimidine synthesis inhibitor.

11252. The method of item 11200 wherein the agent inhibits ribonucleotide synthesis or function.

11253. The method of item 11200 wherein the agent inhibits thymidine monophosphate synthesis or function.

11254. The method of item 11200 wherein the agent inhibits DNA synthesis.

11255. The method of item 11200 wherein the agent causes DNA adduct formation.

11256. The method of item 11200 wherein the agent inhibits protein synthesis.

11257. The method of item 11200 wherein the agent inhibits microtubule function.

11258. The method of item 11200 wherein the agent is a cyclin dependent protein kinase inhibitor.

11259. The method of item 11200 wherein the agent is an epidermal growth factor kinase inhibitor.

11260. The method of item 11200 wherein the agent is an elastase inhibitor.

11261. The method of item 11200 wherein the agent is a factor Xa inhibitor.

11262. The method of item 11200 wherein the agent is a farnesyltransferase inhibitor.

11263. The method of item 11200 wherein the agent is a fibrinogen antagonist.

11264. The method of item 11200 wherein the agent is a guanylate cyclase stimulant.

11265. The method of item 11200 wherein the agent is a heat shock protein 90 antagonist.

11266. The method of item 11200 wherein the agent is a heat shock protein 90 antagonist, wherein the heat shock protein 90 antagonist is geldanamycin or an analogue or derivative thereof.

11267. The method of item 11200 wherein the agent is a guanylate cyclase stimulant.

11268. The method of item 11200 wherein the agent is a HMGCoA reductase inhibitor.

11269. The method of item 11200 wherein the agent is a HMGCoA reductase inhibitor, wherein the HMGCoA reductase inhibitor is simvastatin or an analogue or derivative thereof.

11270. The method of item 11200 wherein the agent is a hydroorotate dehydrogenase inhibitor.

11271. The method of item 11200 wherein the agent is an IKK2 inhibitor.

11272. The method of item 11200 wherein the agent is an IL-1 antagonist.

11273. The method of item 11200 wherein the agent is an ICE antagonist.

11274. The method of item 11200 wherein the agent is an IRAK antagonist.

11275. The method of item 11200 wherein the agent is an IL-4 agonist.

11276. The method of item 11200 wherein the agent is an immunomodulatory agent.

11277. The method of item 11200 wherein the agent is sirolimus or an analogue or derivative thereof.

11278. The method of item 11200 wherein the agent is not sirolimus.

11279. The method of item 11200 wherein the agent is everolimus or an analogue or derivative thereof.

11280. The method of item 11200 wherein the agent is tacrolimus or an analogue or derivative thereof.

11281. The method of item 11200 wherein the agent is not tacrolimus.

11282. The method of item 11200 wherein the agent is biolmus or an analogue or derivative thereof.

11283. The method of item 11200 wherein the agent is tresperimus or an analogue or derivative thereof.

11284. The method of item 11200 wherein the agent is auranofin or an analogue or derivative thereof.

11285. The method of item 11200 wherein the agent is 27-0-demethylrapamycin or an analogue or derivative thereof.

11286. The method of item 11200 wherein the agent is gusperimus or an analogue or derivative thereof.

11287. The method of item 11200 wherein the agent is pimecrolimus or an analogue or derivative thereof.

11288. The method of item 11200 wherein the agent is ABT-578 or an analogue or derivative thereof.

11289. The method of item 11200 wherein the agent is an inosine monophosphate dehydrogenase (IMPDH) inhibitor.

11290. The method of item 11200 wherein the agent is an IMPDH inhibitor, wherein the IMPDH inhibitor is mycophenolic acid or an analogue or derivative thereof.

11291. The method of item 11200 wherein the agent is an IMPDH inhibitor, wherein the IMPDH inhibitor is 1-alpha-25 dihydroxy vitamin D₃ or an analogue or derivative thereof.

11292. The method of item 11200 wherein the agent is a leukotriene inhibitor.

11293. The method of item 11200 wherein the agent is a MCP-1 antagonist.

11294. The method of item 11200 wherein the agent is a MMP inhibitor.

11295. The method of item 11200 wherein the agent is an NF kappa B inhibitor.

11296. The method of item 11200 wherein the agent is an NF kappa B inhibitor, wherein the NF kappa B inhibitor is Bay 11-7082.

11297. The method of item 11200 wherein the agent is an NO agonist.

11298. The method of item 11200 wherein the agent is a p38 MAP kinase inhibitor.

11299. The method of item 11200 wherein the agent is a p38 MAP kinase inhibitor, wherein the p38 MAP kinase inhibitor is SB 202190.

11300. The method of item 11200 wherein the agent is a phosphodiesterase inhibitor.

11301. The method of item 11200 wherein the agent is a TGF beta inhibitor.

11302. The method of item 11200 wherein the agent is a thromboxane A2 antagonist.

11303. The method of item 11200 wherein the agent is a TNFa antagonist.

11304. The method of item 11200 wherein the agent is a TACE inhibitor.

11305. The method of item 11200 wherein the agent is a tyrosine kinase inhibitor.

11306. The method of item 11200 wherein the agent is a vitronectin inhibitor.

11307. The method of item 11200 wherein the agent is a fibroblast growth factor inhibitor.

11308. The method of item 11200 wherein the agent is a protein kinase inhibitor.

11309. The method of item 11200 wherein the agent is a PDGF receptor kinase inhibitor.

11310. The method of item 11200 wherein the agent is an endothelial growth factor receptor kinase inhibitor.

11311. The method of item 11200 wherein the agent is a retinoic acid receptor antagonist.

11312. The method of item 11200 wherein the agent is a platelet derived growth factor receptor kinase inhibitor.

11313. The method of item 11200 wherein the agent is a fibronogin antagonist.

11314. The method of item 11200 wherein the agent is an antimycotic agent.

11315. The method of item 11200 wherein the agent is an antimycotic agent, wherein the antimycotic agent is sulconizole.

11316. The method of item 11200 wherein the agent is a bisphosphonate.

11317. The method of item 11200 wherein the agent is a phospholipase A1 inhibitor.

11318. The method of item 11200 wherein the agent is a histamine H1/H2/H3 receptor antagonist.

11319. The method of item 11200 wherein the agent is a macrolide antibiotic.

11320. The method of item 11200 wherein the agent is a GPIIb/IIIa receptor antagonist.

11321. The method of item 11200 wherein the agent is an endothelin receptor antagonist.

11322. The method of item 11200 wherein the agent is a peroxisome proliferator-activated receptor agonist.

11323. The method of item 11200 wherein the agent is an estrogen receptor agent.

11324. The method of item 11200 wherein the agent is a somastostatin analogue.

11325. The method of item 11200 wherein the agent is a neurokinin 1 antagonist.

11326. The method of item 11200 wherein the agent is a neurokinin 3 antagonist.

11327. The method of item 11200 wherein the agent is a VLA-4 antagonist.

11328. The method of item 11200 wherein the agent is an osteoclast inhibitor.

11329. The method of item 11200 wherein the agent is a DNA topoisomerase ATP hydrolyzing inhibitor.

11330. The method of item 11200 wherein the agent is an angiotensin I converting enzyme inhibitor.

11331. The method of item 11200 wherein the agent is an angiotensin II antagonist.

11332. The method of item 11200 wherein the agent is an enkephalinase inhibitor.

11333. The method of item 11200 wherein the agent is a peroxisome proliferator-activated receptor gamma agonist insulin sensitizer.

11334. The method of item 11200 wherein the agent is a protein kinase C inhibitor.

11335. The method of item 11200 wherein the agent is a ROCK (rho-associated kinase) inhibitor.

11336. The method of item 11200 wherein the agent is a CXCR3 inhibitor.

11337. The method of item 11200 wherein the agent is an ltk inhibitor.

11338. The method of item 11200 wherein the agent is a cytosolic phospholipase A₂-alpha inhibitor.

11339. The method of item 11200 wherein the agent is a PPAR agonist.

11340. The method of item 11200 wherein the agent is an immunosuppressant.

11341. The method of item 11200 wherein the agent is an Erb inhibitor.

11342. The method of item 11200 wherein the agent is an apoptosis agonist.

11343. The method of item 11200 wherein the agent is a lipocortin agonist.

11344. The method of item 11200 wherein the agent is a VCAM-1 antagonist.

11345. The method of item 11200 wherein the agent is a collagen antagonist.

11346. The method of item 11200 wherein the agent is an alpha 2 integrin antagonist.

11347. The method of item 11200 wherein the agent is a TNF alpha inhibitor.

11348. The method of item 11200 wherein the agent is a nitric oxide inhibitor.

11349. The method of item 11200 wherein the agent is a cathepsin inhibitor.

11350. The method of item 11200 wherein the agent is not an anti-inflammatory agent.

11351. The method of item 11200 wherein the agent is not a steroid.

11352. The method of item 11200 wherein the agent is not a glucocorticosteroid.

11353. The method of item 11200 wherein the agent is not dexamethasone.

11354. The method of item 11200 wherein the agent is not an anti-infective agent.

11355. The method of item 11200 wherein the agent is not an antibiotic.

11356. The method of item 11200 wherein the agent is not an anti-fungal agent.

11357. The method of item 11200, wherein the composition comprises a polymer.

11358. The method of item 11200, wherein the composition comprises a polymeric carrier.

11359. The method of item 11200 wherein the anti-scarring agent inhibits adhesion between the device and a host into which the device is implanted.

11360. The method of item 11200 wherein the device delivers the anti-scarring agent locally to tissue proximate to the device.

11361. The method of item 11200 wherein the device has a coating that comprises the anti-scarring agent.

11362. The method of item 11200, wherein the device has a coating that comprises the agent and is disposed on a surface of the implant.

11363. The method of item 11200, wherein the device has a coating that comprises the agent and directly contacts the implant.

11364. The method of item 11200, wherein the device has a coating that comprises the agent and indirectly contacts the implant.

11365. The method of item 11200, wherein the device has a coating that comprises the agent and partially covers the implant.

11366. The method of item 11200, wherein the device has a coating that comprises the agent and completely covers the implant.

11367. The method of item 11200, wherein the device has a uniform coating.

11368. The method of item 11200, wherein the device has a non-uniform coating.

11369. The method of item 11200, wherein the device has a discontinuous coating.

11370. The method of item 11200, wherein the device has a patterned coating.

11371. The method of item 11200, wherein the device has a coating with a thickness of 100 μm or less.

11372. The method of item 11200, wherein the device has a coating with a thickness of 10 μm or less.

11373. The method of item 11200, wherein the device has a coating, and the coating adheres to the surface of the implant upon deployment of the implant.

11374. The method of item 11200, wherein the device has a coating, and wherein the coating is stable at room temperature for a period of 1 year.

11375. The method of item 11200, wherein the device has a coating, and wherein the anti-scarring agent is present in the coating in an amount ranging between about 0.0001% to about 1% by weight.

11376. The method of item 11200, wherein the device has a coating, and wherein the anti-scarring agent is present in the coating in an amount ranging between about 1% to about 10% by weight.

11377. The method of item 11200, wherein the device has a coating, and wherein the anti-scarring agent is present in the coating in an amount ranging between about 10% to about 25% by weight.

11378. The method of item 11200, wherein the device has a coating, and wherein the anti-scarring agent is present in the coating in an amount ranging between about 25% to about 70% by weight.

11379. The method of item 11200, wherein the device has a coating, and wherein the coating further comprises a polymer.

11380. The method of item 11200, wherein the device has a first coating having a first composition and a second coating having a second composition.

11381. The method of item 11200, wherein the device has a first coating having a first composition and a second coating having a second composition, wherein the first composition and the second composition are different.

11382. The method of item 11200, wherein the composition comprises a polymer.

11383. The method of item 11200, wherein the composition comprises a polymeric carrier.

11384. The method of item 11200, wherein the composition comprises a polymeric carrier, and wherein the polymeric carrier comprises a copolymer.

11385. The method of item 11200, wherein the composition comprises a polymeric carrier, and wherein the polymeric carrier comprises a block copolymer.

11386. The method of item 11200, wherein the composition comprises a polymeric carrier, and wherein the polymeric carrier comprises a random copolymer.

11387. The method of item 11200, wherein the composition comprises a polymeric carrier, and wherein the polymeric carrier comprises a biodegradable polymer.

11388. The method of item 11200, wherein the composition comprises a polymeric carrier, and wherein the polymeric carrier comprises a non-biodegradable polymer.

11389. The method of item 11200, wherein the composition comprises a polymeric carrier, and wherein the polymeric carrier comprises a hydrophilic polymer.

11390. The method of item 11200, wherein the composition comprises a polymeric carrier, and wherein the polymeric carrier comprises a hydrophobic polymer.

11391. The method of item 11200, wherein the composition comprises a polymeric carrier, and wherein the polymeric carrier comprises a polymer having hydrophilic domains.

11392. The method of item 11200, wherein the composition comprises a polymeric carrier, and wherein the polymeric carrier comprises a polymer having hydrophobic domains.

11393. The method of item 11200, wherein the composition comprises a polymeric carrier, and wherein the polymeric carrier comprises a non-conductive polymer.

11394. The method of item 11200, wherein the composition comprises a polymeric carrier, and wherein the polymeric carrier comprises an elastomer.

11395. The method of item 11200, wherein the composition comprises a polymeric carrier, and wherein the polymeric carrier comprises a hydrogel.

11396. The method of item 11200, wherein the composition comprises a polymeric carrier, and wherein the polymeric carrier comprises a silicone polymer.

11397. The method of item 11200, wherein the composition comprises a polymeric carrier, and wherein the polymeric carrier comprises a hydrocarbon polymer.

11398. The method of item 11200, wherein the composition comprises a polymeric carrier, and wherein the polymeric carrier comprises a styrene-derived polymer.

11399. The method of item 11200, wherein the composition comprises a polymeric carrier, and wherein the polymeric carrier comprises a butadiene polymer.

11400. The method of item 11200, wherein the composition comprises a polymeric carrier, and wherein the polymeric carrier comprises a macromer.

11401. The method of item 11200, wherein the composition comprises a polymeric carrier, and wherein the polymeric carrier comprises a poly(ethylene glycol)polymer.

11402. The method of item 11200 wherein the composition comprises a polymeric carrier, and wherein the polymeric carrier comprises an amorphous polymer.

11403. The method of item 11200, wherein the device comprises a lubricious coating.

11404. The method of item 11200 wherein the anti-scarring agent is located within pores or holes of the device.

11405. The method of item 11200 wherein the anti-scarring agent is located within a channel, lumen, or divet of the device.

11406. The method of item 11200, wherein the device comprises a second pharmaceutically active agent.

11407. The method of item 11200 wherein the device comprises an anti-inflammatory agent.

11408. The method of item 11200 wherein the device comprises an agent that inhibits infection.

11409. The method of item 11200 wherein the device comprises an agent that inhibits infection, and wherein the agent is an anthracycline.

11410. The method of item 11200 wherein the device comprises an agent that inhibits infection, and wherein the agent is doxorubicin.

11411. The method of item 11200 wherein the device comprises an agent that inhibits infection, and wherein the agent is mitoxantrone.

11412. The method of item 11200 wherein the device comprises an agent that inhibits infection, and wherein the agent is a fluoropyrimidine.

11413. The method of item 11200 wherein the device comprises an agent that inhibits infection, and wherein the agent is 5-fluorouracil (5-FU).

11414. The method of item 11200 wherein the device comprises an agent that inhibits infection, and wherein the agent is a folic acid antagonist.

11415. The method of item 11200 wherein the device comprises an agent that inhibits infection, and wherein the agent is methotrexate.

11416. The method of item 11200 wherein the device comprises an agent that inhibits infection, and wherein the agent is a podophylotoxin.

11417. The method of item 11200 wherein the device comprises an agent that inhibits infection, and wherein the agent is etoposide.

11418. The method of item 11200 wherein the device comprises an agent that inhibits infection, and wherein the agent is a camptothecin.

11419. The method of item 11200 wherein the device comprises an agent that inhibits infection, and wherein the agent is a hydroxyurea.

11420. The method of item 11200 wherein the device comprises an agent that inhibits infection, and wherein the agent is a platinum complex.

11421. The method of item 11200 wherein the device comprises an agent that inhibits infection, and wherein the agent is cisplatin.

11422. The method of item 11200, further comprising an anti-thrombotic agent.

11423. The method of item 11200 wherein the device comprises a visualization agent.

11424. The method of item 11200 wherein the device comprises a visualization agent, wherein the visualization agent is a radiopaque material, and wherein the radiopaque material comprises a metal, a halogenated compound, or a barium containing compound.

11425. The method of item 11200 wherein the device comprises a visualization agent, wherein the visualization agent is a radiopaque material, and wherein the radiopaque material comprises barium, tantalum, or technetium.

11426. The method of item 11200 wherein the device comprises a visualization agent, and wherein the visualization agent is a MRI responsive material.

11427. The method of item 11200 wherein the device comprises a visualization agent, and wherein the visualization agent comprises a gadolinium chelate.

11428. The method of item 11200 wherein the device comprises a visualization agent, and wherein the visualization agent comprises iron, magnesium, manganese, copper, or chromium.

11429. The method of item 11200 wherein the device comprises a visualization agent, and wherein the visualization agent comprises an iron oxide compound.

11430. The method of item 11200 wherein the device comprises a visualization agent, and wherein the visualization agent comprises a dye, pigment, or colorant.

11431. The method of item 11200 wherein the device comprises an echogenic material.

11432. The method of item 11200 wherein the device comprises an echogenic material, and wherein the echogenic material is in the form of a coating.

11433. The method of item 11200 wherein the device is sterile.

11434. The method of item 11200 wherein the anti-scarring agent is released into tissue in the vicinity of the device after deployment of the device.

11435. The method of item 11200 wherein the anti-scarring agent is released into tissue in the vicinity of the device after deployment of the device, and wherein the tissue is connective tissue.

11436. The method of item 11200 wherein the anti-scarring agent is released into tissue in the vicinity of the device after deployment of the device, and wherein the tissue is muscle tissue.

11437. The method of item 11200 wherein the anti-scarring agent is released into tissue in the vicinity of the device after deployment of the device, and wherein the tissue is nerve tissue.

11438. The method of item 11200 wherein the anti-scarring agent is released into tissue in the vicinity of the device after deployment of the device, and wherein the tissue is epithelium tissue.

11439. The method of item 11200 wherein the anti-scarring agent is released in effective concentrations from the device over a period ranging from the time of deployment of the device to about 1 year.

11440. The method of item 11200 wherein the anti-scarring agent is released in effective concentrations from the device over a period ranging from about 1 month to 6 months.

11441. The method of item 11200 wherein the anti-scarring agent is released in effective concentrations from the device over a period ranging from about 1-90 days.

11442. The method of item 11200 wherein the anti-scarring agent is released in effective concentrations from the device at a constant rate.

11443. The method of item 11200 wherein the anti-scarring agent is released in effective concentrations from the device at an increasing rate.

11444. The method of item 11200 wherein the anti-scarring agent is released in effective concentrations from the device at a decreasing rate.

11445. The method of item 11200 wherein the anti-scarring agent is released in effective concentrations from the composition comprising the anti-scarring agent by diffusion over a period ranging from the time of deployment of the device to about 90 days.

11446. The method of item 11200 wherein the anti-scarring agent is released in effective concentrations from the composition comprising the anti-scarring agent by erosion of the composition over a period ranging from the time of deployment of the device to about 90 days.

11447. The method of item 11200 wherein the device comprises about 0.01 μg to about 10 μg of the anti-scarring agent.

11448. The method of item 11200 wherein the device comprises about 10 μg to about 10 mg of the anti-scarring agent.

11449. The method of item 11200 wherein the device comprises about 10 mg to about 250 mg of the anti-scarring agent.

11450. The method of item 11200 wherein the device comprises about 250 mg to about 1000 mg of the anti-scarring agent.

11451. The method of item 11200 wherein the device comprises about 1000 mg to about 2500 mg of the anti-scarring agent.

11452. The method of item 11200 wherein a surface of the device comprises less than 0.01 μg of the anti-scarring agent per mm² of device surface to which the anti-scarring agent is applied.

11453. The method of item 11200 wherein a surface of the device comprises about 0.01 μg to about 1 μg of the anti-scarring agent per mm² of device surface to which the anti-scarring agent is applied.

11454. The method of item 11200 wherein a surface of the device comprises about 1 μg to about 10 μg of the anti-scarring agent per mm² of device surface to which the anti-scarring agent is applied.

11455. The method of item 11200 wherein a surface of the device comprises about 10 μg to about 250 μg of the anti-scarring agent per mm² of device surface to which the anti-scarring agent is applied.

11456. The method of item 11200 wherein a surface of the device comprises about 250 μg to about 1000 μg of the anti-scarring agent of anti-scarring agent per mm² of device surface to which the anti-scarring agent is applied.

11457. The method of item 11200 wherein a surface of the device comprises about 1000 μg to about 2500 μg of the anti-scarring agent per mm² of device surface to which the anti-scarring agent is applied.

11458. The method of item 11200 wherein the combining is performed by direct affixing the agent or the composition to the implant.

11459. The method of item 11200 wherein the combining is performed by spraying the agent or the component onto the implant.

11460. The method of item 11200 wherein the combining is performed by electrospraying the agent or the composition onto the implant.

11461. The method of item 11200 wherein the combining is performed by dipping the implant into a solution comprising the agent or the composition.

11462. The method of item 11200 wherein the combining is performed by covalently attaching the agent or the composition to the implant.

11463. The method of item 11200 wherein the combining is performed by non-covalently attaching the agent or the composition to the implant.

11464. The method of item 11200 wherein the combining is performed by coating the implant with a substance that contains the agent or the composition.

11465. The method of item 11200 wherein the combining is performed by coating the implant with a substance that absorbs the agent.

11466. The method of item 11200 wherein the combining is performed by interweaving a thread composed of, or coated with, the agent or the composition.

11467. The method of item 11200 wherein the combining is performed by covering all the implant with a sleeve that contains the agent or the composition.

11468. The method of item 11200 wherein the combining is performed by covering a portion of the implant with a sleeve that contains the agent or the composition.

11469. The method of item 11200 wherein the combining is performed by covering all the implant with a cover that contains the agent or the composition.

11470. The method of item 11200 wherein the combining is performed by covering a portion of the implant with a cover that contains the agent or the composition.

11471. The method of item 11200 wherein the combining is performed by covering all the implant with an electrospun fabric that contains the agent or the composition.

11472. The method of item 11200 wherein the combining is performed by covering a portion of the implant with an electrospun fabric that contains the agent or the composition.

11473. The method of item 11200 wherein the combining is performed by covering all the implant with a mesh that contains the agent or the composition.

11474. The method of item 11200 wherein the combining is performed by covering a portion of the implant with a mesh that contains the agent or the composition.

11475. The method of item 11200 wherein the combining is performed by constructing all the implant with the agent or the composition.

11476. The method of item 11200 wherein the combining is performed by constructing a portion of the implant with the agent or the composition.

11477. The method of item 11200 wherein the combining is performed by impregnating the implant with the agent or the composition.

11478. The method of item 11200 wherein the combining is performed by constructing all of the implant from a degradable polymer that releases the agent.

11479. The method of item 11200 wherein the combining is performed by constructing a portion of the implant from a degradable polymer that releases the agent.

11480. The method of item 11200 wherein the combining is performed by dipping the implant into a solution that comprise the agent and an inert solvent for the implant.

11481. The method of item 11200 wherein the combining is performed by dipping the implant into a solution that comprises the agent and a solvent that will swill the implant.

11482. The method of item 11200 wherein the combining is performed by dipping the implant into a solution that comprises the agent and a solvent that will dissolve the implant.

11483. The method of item 11200 wherein the combining is performed by dipping the implant into a solution that comprises the agent, a polymer and an inert solvent for the implant.

11484. The method of item 11200 wherein the combining is performed by dipping the implant into a solution that comprises the agent, a polymer and a solvent that will swill the implant.

11485. The method of item 11200 wherein the combining is performed by dipping the implant into a solution that comprises the agent, a polymer and a solvent that will dissolve the implant.

11486. The method of item 11200 wherein the combining is performed by spraying the implant into a solution that comprises the agent and an inert solvent for the implant.

11487. The method of item 11200 wherein the combining is performed by spraying the implant into a solution that comprises the agent and a solvent that will swill the implant.

11488. The method of item 11200 wherein the combining is performed by spraying the implant into a solution that comprises the agent and a solvent that will dissolve the implant.

11489. The method of item 11200 wherein the combining is performed by spraying the implant into a solution that comprises the agent, a polymer and an inert solvent for the implant.

11490. The method of item 11200 wherein the combining is performed by spraying the implant into a solution that comprises the agent, a polymer and a solvent that will swill the implant.

11491. The method of item 11200 wherein the combining is performed by spraying the implant into a solution that comprises the agent, a polymer and a solvent that will dissolve the implant.

11492. The method of item 11200 wherein the implant is an AV fistula.

11493. The method of item 11200 wherein the implant is an AV access graft.

11494. The method of item 11200 wherein the implant is a venous catheter.

11495. The method of item 11200 wherein the implant is an implantable port.

11496. The method of item 11200 wherein the implant is an AV shunt.

11497. A method of making a medical device comprising: combining an implant that provides an anastomotic connection (i.e., an anastomotic connector device) and an anti-scarring agent or a composition comprising an anti-scarring agent, wherein the agent inhibits scarring between the device and a host into which the device is implanted.

11498. The method of item 11497 wherein the agent inhibits cell regeneration.

11499. The method of item 11497 wherein the agent inhibits angiogenesis.

11500. The method of item 11497 wherein the agent inhibits fibroblast migration.

11501. The method of item 11497 wherein the agent inhibits fibroblast proliferation.

11502. The method of item 11497 wherein the agent inhibits deposition of extracellular matrix.

11503. The method of item 11497 wherein the agent inhibits tissue remodeling.

11504. The method of item 11497 wherein the agent is an angiogenesis inhibitor.

11505. The method of item 11497 wherein the agent is a 5-lipoxygenase inhibitor or antagonist.

11506. The method of item 11497 wherein the agent is a chemokine receptor antagonist.

11507. The method of item 11497 wherein the agent is a cell cycle inhibitor.

11508. The method of item 11497 wherein the agent is a taxane.

11509. The method of item 11497 wherein the agent is an anti-microtubule agent.

11510. The method of item 11497 wherein the agent is paclitaxel.

11511. The method of item 11497 wherein the agent is not paclitaxel.

11512. The method of item 11497 wherein the agent is an analogue or derivative of paclitaxel.

11513. The method of item 11497 wherein the agent is a vinca alkaloid.

11514. The method of item 11497 wherein the agent is camptothecin or an analogue or derivative thereof.

11515. The method of item 11497 wherein the agent is a podophyllotoxin.

11516. The method of item 11497 wherein the agent is a podophyllotoxin, wherein the podophyllotoxin is etoposide or an analogue or derivative thereof.

11517. The method of item 11497 wherein the agent is an anthracycline.

11518. The method of item 11497 wherein the agent is an anthracycline, wherein the anthracycline is doxorubicin or an analogue or derivative thereof.

11519. The method of item 11497 wherein the agent is an anthracycline, wherein the anthracycline is mitoxantrone or an analogue or derivative thereof.

11520. The method of item 11497 wherein the agent is a platinum compound.

11521. The method of item 11497 wherein the agent is a nitrosourea.

11522. The method of item 11497 wherein the agent is a nitroimidazole.

11523. The method of item 11497 wherein the agent is a folic acid antagonist.

11524. The method of item 11497 wherein the agent is a cytidine analogue.

11525. The method of item 11497 wherein the agent is a pyrimidine analogue.

11526. The method of item 11497 wherein the agent is a fluoropyrimidine analogue.

11527. The method of item 11497 wherein the agent is a purine analogue.

11528. The method of item 11497 wherein the agent is a nitrogen mustard or an analogue or derivative thereof.

11529. The method of item 11497 wherein the agent is a hydroxyurea.

11530. The method of item 11497 wherein the agent is a mytomicin or an analogue or derivative thereof.

11531. The method of item 11497 wherein the agent is an alkyl sulfonate.

11532. The method of item 11497 wherein the agent is a benzamide or an analogue or derivative thereof.

11533. The method of item 11497 wherein the agent is a nicotinamide or an analogue or derivative thereof.

11534. The method of item 11497 wherein the agent is a halogenated sugar or an analogue or derivative thereof.

11535. The method of item 11497 wherein the agent is a DNA alkylating agent.

11536. The method of item 11497 wherein the agent is an anti-microtubule agent.

11537. The method of item 11497 wherein the agent is a topoisomerase inhibitor.

11538. The method of item 11497 wherein the agent is a DNA cleaving agent.

11539. The method of item 11497 wherein the agent is an antimetabolite.

11540. The method of item 11497 wherein the agent inhibits adenosine deaminase.

11541. The method of item 11497 wherein the agent inhibits purine ring synthesis.

11542. The method of item 11497 wherein the agent is a nucleotide interconversion inhibitor.

11543. The method of item 11497 wherein the agent inhibits dihydrofolate reduction.

11544. The method of item 11497 wherein the agent blocks thymidine monophosphate.

11545. The method of item 11497 wherein the agent causes DNA damage.

11546. The method of item 11497 wherein the agent is a DNA intercalation agent.

11547. The method of item 11497 wherein the agent is a RNA synthesis inhibitor.

11548. The method of item 11497 wherein the agent is a pyrimidine synthesis inhibitor.

11549. The method of item 11497 wherein the agent inhibits ribonucleotide synthesis or function.

11550. The method of item 11497 wherein the agent inhibits thymidine monophosphate synthesis or function.

11551. The method of item 11497 wherein the agent inhibits DNA synthesis.

11552. The method of item 11497 wherein the agent causes DNA adduct formation.

11553. The method of item 11497 wherein the agent inhibits protein synthesis.

11554. The method of item 11497 wherein the agent inhibits microtubule function.

11555. The method of item 11497 wherein the agent is a cyclin dependent protein kinase inhibitor.

11556. The method of item 11497 wherein the agent is an epidermal growth factor kinase inhibitor.

11557. The method of item 11497 wherein the agent is an elastase inhibitor.

11558. The method of item 11497 wherein the agent is a factor Xa inhibitor.

11559. The method of item 11497 wherein the agent is a farnesyltransferase inhibitor.

11560. The method of item 11497 wherein the agent is a fibrinogen antagonist.

11561. The method of item 11497 wherein the agent is a guanylate cyclase stimulant.

11562. The method of item 11497 wherein the agent is a heat shock protein 90 antagonist.

11563. The method of item 11497 wherein the agent is a heat shock protein 90 antagonist, wherein the heat shock protein 90 antagonist is geldanamycin or an analogue or derivative thereof.

11564. The method of item 11497 wherein the agent is a guanylate cyclase stimulant.

11565. The method of item 11497 wherein the agent is a HMGCoA reductase inhibitor.

11566. The method of item 11497 wherein the agent is a HMGCoA reductase inhibitor, wherein the HMGCoA reductase inhibitor is simvastatin or an analogue or derivative thereof.

11567. The method of item 11497 wherein the agent is a hydroorotate dehydrogenase inhibitor.

11568. The method of item 11497 wherein the agent is an IKK2 inhibitor.

11569. The method of item 11497 wherein the agent is an IL-1 antagonist.

11570. The method of item 11497 wherein the agent is an ICE antagonist.

11571. The method of item 11497 wherein the agent is an IRAK antagonist.

11572. The method of item 11497 wherein the agent is an IL-4 agonist.

11573. The method of item 11497 wherein the agent is an immunomodulatory agent.

11574. The method of item 11497 wherein the agent is sirolimus or an analogue or derivative thereof.

11575. The method of item 11497 wherein the agent is not sirolimus.

11576. The method of item 11497 wherein the agent is everolimus or an analogue or derivative thereof.

11577. The method of item 11497 wherein the agent is tacrolimus or an analogue or derivative thereof.

11578. The method of item 11497 wherein the agent is not tacrolimus.

11579. The method of item 11497 wherein the agent is biolmus or an analogue or derivative thereof.

11580. The method of item 11497 wherein the agent is tresperimus or an analogue or derivative thereof.

11581. The method of item 11497 wherein the agent is auranofin or an analogue or derivative thereof.

11582. The method of item 11497 wherein the agent is 27-0-demethylrapamycin or an analogue or derivative thereof.

11583. The method of item 11497 wherein the agent is gusperimus or an analogue or derivative thereof.

11584. The method of item 11497 wherein the agent is pimecrolimus or an analogue or derivative thereof.

11585. The method of item 11497 wherein the agent is ABT-578 or an analogue or derivative thereof.

11586. The method of item 11497 wherein the agent is an inosine monophosphate dehydrogenase (IMPDH) inhibitor.

11587. The method of item 11497 wherein the agent is an IMPDH inhibitor, wherein the IMPDH inhibitor is mycophenolic acid or an analogue or derivative thereof.

11588. The method of item 11497 wherein the agent is an IMPDH inhibitor, wherein the IMPDH inhibitor is 1-alpha-25 dihydroxy vitamin D₃ or an analogue or derivative thereof.

11589. The method of item 11497 wherein the agent is a leukotriene inhibitor.

11590. The method of item 11497 wherein the agent is a MCP-1 antagonist.

11591. The method of item 11497 wherein the agent is a MMP inhibitor.

11592. The method of item 11497 wherein the agent is an NF kappa B inhibitor.

11593. The method of item 11497 wherein the agent is an NF kappa B inhibitor, wherein the NF kappa B inhibitor is Bay 11-7082.

11594. The method of item 11497 wherein the agent is an NO agonist.

11595. The method of item 11497 wherein the agent is a p38 MAP kinase inhibitor.

11596. The method of item 11497 wherein the agent is a p38 MAP kinase inhibitor, wherein the p38 MAP kinase inhibitor is SB 202190.

11597. The method of item 11497 wherein the agent is a phosphodiesterase inhibitor.

11598. The method of item 11497 wherein the agent is a TGF beta inhibitor.

11599. The method of item 11497 wherein the agent is a thromboxane A2 antagonist.

11600. The method of item 11497 wherein the agent is a TNFa antagonist.

11601. The method of item 11497 wherein the agent is a TACE inhibitor.

11602. The method of item 11497 wherein the agent is a tyrosine kinase inhibitor.

11603. The method of item 11497 wherein the agent is a vitronectin inhibitor.

11604. The method of item 11497 wherein the agent is a fibroblast growth factor inhibitor.

11605. The method of item 11497 wherein the agent is a protein kinase inhibitor.

11606. The method of item 11497 wherein the agent is a PDGF receptor kinase inhibitor.

11607. The method of item 11497 wherein the agent is an endothelial growth factor receptor kinase inhibitor.

11608. The method of item 11497 wherein the agent is a retinoic acid receptor antagonist.

11609. The method of item 11497 wherein the agent is a platelet derived growth factor receptor kinase inhibitor.

11610. The method of item 11497 wherein the agent is a fibronogin antagonist.

11611. The method of item 11497 wherein the agent is an antimycotic agent.

11612. The method of item 11497 wherein the agent is an antimycotic agent, wherein the antimycotic agent is sulconizole.

11613. The method of item 11497 wherein the agent is a bisphosphonate.

11614. The method of item 11497 wherein the agent is a phospholipase A1 inhibitor.

11615. The method of item 11497 wherein the agent is a histamine H1/H2/H3 receptor antagonist.

11616. The method of item 11497 wherein the agent is a macrolide antibiotic.

11617. The method of item 11497 wherein the agent is a GPIIb/IIIa receptor antagonist.

11618. The method of item 11497 wherein the agent is an endothelin receptor antagonist.

11619. The method of item 11497 wherein the agent is a peroxisome proliferator-activated receptor agonist.

11620. The method of item 11497 wherein the agent is an estrogen receptor agent.

11621. The method of item 11497 wherein the agent is a somastostatin analogue.

11622. The method of item 11497 wherein the agent is a neurokinin 1 antagonist.

11623. The method of item 11497 wherein the agent is a neurokinin 3 antagonist.

11624. The method of item 11497 wherein the agent is a VLA-4 antagonist.

11625. The method of item 11497 wherein the agent is an osteoclast inhibitor.

11626. The method of item 11497 wherein the agent is a DNA topoisomerase ATP hydrolyzing inhibitor.

11627. The method of item 11497 wherein the agent is an angiotensin I converting enzyme inhibitor.

11628. The method of item 11497 wherein the agent is an angiotensin II antagonist.

11629. The method of item 11497 wherein the agent is an enkephalinase inhibitor.

11630. The method of item 11497 wherein the agent is a peroxisome proliferator-activated receptor gamma agonist insulin sensitizer.

11631. The method of item 11497 wherein the agent is a protein kinase C inhibitor.

11632. The method of item 11497 wherein the agent is a ROCK (rho-associated kinase) inhibitor.

11633. The method of item 11497 wherein the agent is a CXCR3 inhibitor.

11634. The method of item 11497 wherein the agent is an Itk inhibitor.

11635. The method of item 11497 wherein the agent is a cytosolic phospholipase A₂-alpha inhibitor.

11636. The method of item 11497 wherein the agent is a PPAR agonist.

11637. The method of item 11497 wherein the agent is an immunosuppressant.

11638. The method of item 11497 wherein the agent is an Erb inhibitor.

11639. The method of item 11497 wherein the agent is an apoptosis agonist.

11640. The method of item 11497 wherein the agent is a lipocortin agonist.

11641. The method of item 11497 wherein the agent is a VCAM-1 antagonist.

11642. The method of item 11497 wherein the agent is a collagen antagonist.

11643. The method of item 11497 wherein the agent is an alpha 2 integrin antagonist.

11644. The method of item 11497 wherein the agent is a TNF alpha inhibitor.

11645. The method of item 11497 wherein the agent is a nitric oxide inhibitor.

11646. The method of item 11497 wherein the agent is a cathepsin inhibitor.

11647. The method of item 11497 wherein the agent is not an anti-inflammatory agent.

11648. The method of item 11497 wherein the agent is not a steroid.

11649. The method of item 11497 wherein the agent is not a glucocorticosteroid.

11650. The method of item 11497 wherein the agent is not dexamethasone.

11651. The method of item 11497 wherein the agent is not an anti-infective agent.

11652. The method of item 11497 wherein the agent is not an antibiotic.

11653. The method of item 11497 wherein the agent is not an anti-fungal agent.

11654. The method of item 11497, wherein the composition comprises a polymer.

11655. The method of item 11497, wherein the composition comprises a polymeric carrier.

11656. The method of item 11497 wherein the anti-scarring agent inhibits adhesion between the device and a host into which the device is implanted.

11657. The method of item 11497 wherein the device delivers the anti-scarring agent locally to tissue proximate to the device.

11658. The method of item 11497 wherein the device has a coating that comprises the anti-scarring agent.

11659. The method of item 11497, wherein the device has a coating that comprises the agent and is disposed on a surface of the implant.

11660. The method of item 11497, wherein the device has a coating that comprises the agent and directly contacts the implant.

11661. The method of item 11497, wherein the device has a coating that comprises the agent and indirectly contacts the implant.

11662. The method of item 11497, wherein the device has a coating that comprises the agent and partially covers the implant.

11663. The method of item 11497, wherein the device has a coating that comprises the agent and completely covers the implant.

11664. The method of item 11497, wherein the device has a uniform coating.

11665. The method of item 11497, wherein the device has a non-uniform coating.

11666. The method of item 11497, wherein the device has a discontinuous coating.

11667. The method of item 11497, wherein the device has a patterned coating.

11668. The method of item 11497, wherein the device has a coating with a thickness of 100 μm or less.

11669. The method of item 11497, wherein the device has a coating with a thickness of 10 μm or less.

11670. The method of item 11497, wherein the device has a coating, and the coating adheres to the surface of the implant upon deployment of the implant.

11671. The method of item 11497, wherein the device has a coating, and wherein the coating is stable at room temperature for a period of 1 year.

11672. The method of item 11497, wherein the device has a coating, and wherein the anti-scarring agent is present in the coating in an amount ranging between about 0.0001% to about 1% by weight.

11673. The method of item 11497, wherein the device has a coating, and wherein the anti-scarring agent is present in the coating in an amount ranging between about 1% to about 10% by weight.

11674. The method of item 11497, wherein the device has a coating, and wherein the anti-scarring agent is present in the coating in an amount ranging between about 10% to about 25% by weight.

11675. The method of item 11497, wherein the device has a coating, and wherein the anti-scarring agent is present in the coating in an amount ranging between about 25% to about 70% by weight.

11676. The method of item 11497, wherein the device has a coating, and wherein the coating further comprises a polymer.

11677. The method of item 11497, wherein the device has a first coating having a first composition and a second coating having a second composition.

11678. The method of item 11497, wherein the device has a first coating having a first composition and a second coating having a second composition, wherein the first composition and the second composition are different.

11679. The method of item 11497, wherein the composition comprises a polymer.

11680. The method of item 11497, wherein the composition comprises a polymeric carrier.

11681. The method of item 11497, wherein the composition comprises a polymeric carrier, and wherein the polymeric carrier comprises a copolymer.

11682. The method of item 11497, wherein the composition comprises a polymeric carrier, and wherein the polymeric carrier comprises a block copolymer.

11683. The method of item 11497, wherein the composition comprises a polymeric carrier, and wherein the polymeric carrier comprises a random copolymer.

11684. The method of item 11497, wherein the composition comprises a polymeric carrier, and wherein the polymeric carrier comprises a biodegradable polymer.

11685. The method of item 11497, wherein the composition comprises a polymeric carrier, and wherein the polymeric carrier comprises a non-biodegradable polymer.

11686. The method of item 11497, wherein the composition comprises a polymeric carrier, and wherein the polymeric carrier comprises a hydrophilic polymer.

11687. The method of item 11497, wherein the composition comprises a polymeric carrier, and wherein the polymeric carrier comprises a hydrophobic polymer.

11688. The method of item 11497, wherein the composition comprises a polymeric carrier, and wherein the polymeric carrier comprises a polymer having hydrophilic domains.

11689. The method of item 11497, wherein the composition comprises a polymeric carrier, and wherein the polymeric carrier comprises a polymer having hydrophobic domains.

11690. The method of item 11497, wherein the composition comprises a polymeric carrier, and wherein the polymeric carrier comprises a non-conductive polymer.

11691. The method of item 11497, wherein the composition comprises a polymeric carrier, and wherein the polymeric carrier comprises an elastomer.

11692. The method of item 11497, wherein the composition comprises a polymeric carrier, and wherein the polymeric carrier comprises a hydrogel.

11693. The method of item 11497, wherein the composition comprises a polymeric carrier, and wherein the polymeric carrier comprises a silicone polymer.

11694. The method of item 11497, wherein the composition comprises a polymeric carrier, and wherein the polymeric carrier comprises a hydrocarbon polymer.

11695. The method of item 11497, wherein the composition comprises a polymeric carrier, and wherein the polymeric carrier comprises a styrene-derived polymer.

11696. The method of item 11497, wherein the composition comprises a polymeric carrier, and wherein the polymeric carrier comprises a butadiene polymer.

11697. The method of item 11497, wherein the composition comprises a polymeric carrier, and wherein the polymeric carrier comprises a macromer.

11698. The method of item 11497, wherein the composition comprises a polymeric carrier, and wherein the polymeric carrier comprises a poly(ethylene glycol)polymer.

11699. The method of item 11497 wherein the composition comprises a polymeric carrier, and wherein the polymeric carrier comprises an amorphous polymer.

11700. The method of item 11497, wherein the device comprises a lubricious coating.

11701. The method of item 11497 wherein the anti-scarring agent is located within pores or holes of the device.

11702. The method of item 11497 wherein the anti-scarring agent is located within a channel, lumen, or divet of the device.

11703. The method of item 11497, wherein the device comprises a second pharmaceutically active agent.

11704. The method of item 11497 wherein the device comprises an anti-inflammatory agent.

11705. The method of item 11497 wherein the device comprises an agent that inhibits infection.

11706. The method of item 11497 wherein the device comprises an agent that inhibits infection, and wherein the agent is an anthracycline.

11707. The method of item 11497 wherein the device comprises an agent that inhibits infection, and wherein the agent is doxorubicin.

11708. The method of item 11497 wherein the device comprises an agent that inhibits infection, and wherein the agent is mitoxantrone.

11709. The method of item 11497 wherein the device comprises an agent that inhibits infection, and wherein the agent is a fluoropyrimidine.

11710. The method of item 11497 wherein the device comprises an agent that inhibits infection, and wherein the agent is 5-fluorouracil (5-FU).

11711. The method of item 11497 wherein the device comprises an agent that inhibits infection, and wherein the agent is a folic acid antagonist.

11712. The method of item 11497 wherein the device comprises an agent that inhibits infection, and wherein the agent is methotrexate.

11713. The method of item 11497 wherein the device comprises an agent that inhibits infection, and wherein the agent is a podophylotoxin.

11714. The method of item 11497 wherein the device comprises an agent that inhibits infection, and wherein the agent is etoposide.

11715. The method of item 11497 wherein the device comprises an agent that inhibits infection, and wherein the agent is a camptothecin.

11716. The method of item 11497 wherein the device comprises an agent that inhibits infection, and wherein the agent is a hydroxyurea.

11717. The method of item 11497 wherein the device comprises an agent that inhibits infection, and wherein the agent is a platinum complex.

11718. The method of item 11497 wherein the device comprises an agent that inhibits infection, and wherein the agent is cisplatin.

11719. The method of item 11497, further comprising an anti-thrombotic agent.

11720. The method of item 11497 wherein the device comprises a visualization agent.

11721. The method of item 11497 wherein the device comprises a visualization agent, wherein the visualization agent is a radiopaque material, and wherein the radiopaque material comprises a metal, a halogenated compound, or a barium containing compound.

11722. The method of item 11497 wherein the device comprises a visualization agent, wherein the visualization agent is a radiopaque material, and wherein the radiopaque material comprises barium, tantalum, or technetium.

11723. The method of item 11497 wherein the device comprises a visualization agent, and wherein the visualization agent is a MRI responsive material.

11724. The method of item 11497 wherein the device comprises a visualization agent, and wherein the visualization agent comprises a gadolinium chelate.

11725. The method of item 11497 wherein the device comprises a visualization agent, and wherein the visualization agent comprises iron, magnesium, manganese, copper, or chromium.

11726. The method of item 11497 wherein the device comprises a visualization agent, and wherein the visualization agent comprises an iron oxide compound.

11727. The method of item 11497 wherein the device comprises a visualization agent, and wherein the visualization agent comprises a dye, pigment, or colorant.

11728. The method of item 11497 wherein the device comprises an echogenic material.

11729. The method of item 11497 wherein the device comprises an echogenic material, and wherein the echogenic material is in the form of a coating.

11730. The method of item 11497 wherein the device is sterile.

11731. The method of item 11497 wherein the anti-scarring agent is released into tissue in the vicinity of the device after deployment of the device.

11732. The method of item 11497 wherein the anti-scarring agent is released into tissue in the vicinity of the device after deployment of the device, and wherein the tissue is connective tissue.

11733. The method of item 11497 wherein the anti-scarring agent is released into tissue in the vicinity of the device after deployment of the device, and wherein the tissue is muscle tissue.

11734. The method of item 11497 wherein the anti-scarring agent is released into tissue in the vicinity of the device after deployment of the device, and wherein the tissue is nerve tissue.

11735. The method of item 11497 wherein the anti-scarring agent is released into tissue in the vicinity of the device after deployment of the device, and wherein the tissue is epithelium tissue.

11736. The method of item 11497 wherein the anti-scarring agent is released in effective concentrations from the device over a period ranging from the time of deployment of the device to about 1 year.

11737. The method of item 11497 wherein the anti-scarring agent is released in effective concentrations from the device over a period ranging from about 1 month to 6 months.

11738. The method of item 11497 wherein the anti-scarring agent is released in effective concentrations from the device over a period ranging from about 1-90 days.

11739. The method of item 11497 wherein the anti-scarring agent is released in effective concentrations from the device at a constant rate.

11740. The method of item 11497 wherein the anti-scarring agent is released in effective concentrations from the device at an increasing rate.

11741. The method of item 11497 wherein the anti-scarring agent is released in effective concentrations from the device at a decreasing rate.

11742. The method of item 11497 wherein the anti-scarring agent is released in effective concentrations from the composition comprising the anti-scarring agent by diffusion over a period ranging from the time of deployment of the device to about 90 days.

11743. The method of item 11497 wherein the anti-scarring agent is released in effective concentrations from the composition comprising the anti-scarring agent by erosion of the composition over a period ranging from the time of deployment of the device to about 90 days.

11744. The method of item 11497 wherein the device comprises about 0.01 μg to about 10 μg of the anti-scarring agent.

11745. The method of item 11497 wherein the device comprises about 10 μg to about 10 mg of the anti-scarring agent.

11746. The method of item 11497 wherein the device comprises about 10 mg to about 250 mg of the anti-scarring agent.

11747. The method of item 11497 wherein the device comprises about 250 mg to about 1000 mg of the anti-scarring agent.

11748. The method of item 11497 wherein the device comprises about 1000 mg to about 2500 mg of the anti-scarring agent.

11749. The method of item 11497 wherein a surface of the device comprises less than 0.01 μg of the anti-scarring agent per mm² of device surface to which the anti-scarring agent is applied.

11750. The method of item 11497 wherein a surface of the device comprises about 0.01 μg to about 1 μg of the anti-scarring agent per mm² of device surface to which the anti-scarring agent is applied.

11751. The method of item 11497 wherein a surface of the device comprises about 1 μg to about 10 μg of the anti-scarring agent per mm² of device surface to which the anti-scarring agent is applied.

11752. The method of item 11497 wherein a surface of the device comprises about 10 μg to about 250 μg of the anti-scarring agent per mm² of device surface to which the anti-scarring agent is applied.

11753. The method of item 11497 wherein a surface of the device comprises about 250 μg to about 1000 μg of the anti-scarring agent of anti-scarring agent per mm² of device surface to which the anti-scarring agent is applied.

11754. The method of item 11497 wherein a surface of the device comprises about 1000 μg to about 2500 μg of the anti-scarring agent per mm² of device surface to which the anti-scarring agent is applied.

11755. The method of item 11497 wherein the combining is performed by direct affixing the agent or the composition to the implant.

11756. The method of item 11497 wherein the combining is performed by spraying the agent or the component onto the implant.

11757. The method of item 11497 wherein the combining is performed by electrospraying the agent or the composition onto the implant.

11758. The method of item 11497 wherein the combining is performed by dipping the implant into a solution comprising the agent or the composition.

11759. The method of item 11497 wherein the combining is performed by covalently attaching the agent or the composition to the implant.

11760. The method of item 11497 wherein the combining is performed by non-covalently attaching the agent or the composition to the implant.

11761. The method of item 11497 wherein the combining is performed by coating the implant with a substance that contains the agent or the composition.

11762. The method of item 11497 wherein the combining is performed by coating the implant with a substance that absorbs the agent.

11763. The method of item 11497 wherein the combining is performed by interweaving a thread composed of, or coated with, the agent or the composition.

11764. The method of item 11497 wherein the combining is performed by covering all the implant with a sleeve that contains the agent or the composition.

11765. The method of item 11497 wherein the combining is performed by covering a portion of the implant with a sleeve that contains the agent or the composition.

11766. The method of item 11497 wherein the combining is performed by covering all the implant with a cover that contains the agent or the composition.

11767. The method of item 11497 wherein the combining is performed by covering a portion of the implant with a cover that contains the agent or the composition.

11768. The method of item 11497 wherein the combining is performed by covering all the implant with an electrospun fabric that contains the agent or the composition.

11769. The method of item 11497 wherein the combining is performed by covering a portion of the implant with an electrospun fabric that contains the agent or the composition.

11770. The method of item 11497 wherein the combining is performed by covering all the implant with a mesh that contains the agent or the composition.

11771. The method of item 11497 wherein the combining is performed by covering a portion of the implant with a mesh that contains the agent or the composition.

11772. The method of item 11497 wherein the combining is performed by constructing all the implant with the agent or the composition.

11773. The method of item 11497 wherein the combining is performed by constructing a portion of the implant with the agent or the composition.

11774. The method of item 11497 wherein the combining is performed by impregnating the implant with the agent or the composition.

11775. The method of item 11497 wherein the combining is performed by constructing all of the implant from a degradable polymer that releases the agent.

11776. The method of item 11497 wherein the combining is performed by constructing a portion of the implant from a degradable polymer that releases the agent.

11777. The method of item 11497 wherein the combining is performed by dipping the implant into a solution that comprise the agent and an inert solvent for the implant.

11778. The method of item 11497 wherein the combining is performed by dipping the implant into a solution that comprises the agent and a solvent that will swill the implant.

11779. The method of item 11497 wherein the combining is performed by dipping the implant into a solution that comprises the agent and a solvent that will dissolve the implant.

11780. The method of item 11497 wherein the combining is performed by dipping the implant into a solution that comprises the agent, a polymer and an inert solvent for the implant.

11781. The method of item 11497 wherein the combining is performed by dipping the implant into a solution that comprises the agent, a polymer and a solvent that will swill the implant.

11782. The method of item 11497 wherein the combining is performed by dipping the implant into a solution that comprises the agent, a polymer and a solvent that will dissolve the implant.

11783. The method of item 11497 wherein the combining is performed by spraying the implant into a solution that comprises the agent and an inert solvent for the implant.

11784. The method of item 11497 wherein the combining is performed by spraying the implant into a solution that comprises the agent and a solvent that will swill the implant.

11785. The method of item 11497 wherein the combining is performed by spraying the implant into a solution that comprises the agent and a solvent that will dissolve the implant.

11786. The method of item 11497 wherein the combining is performed by spraying the implant into a solution that comprises the agent, a polymer and an inert solvent for the implant.

11787. The method of item 11497 wherein the combining is performed by spraying the implant into a solution that comprises the agent, a polymer and a solvent that will swill the implant.

11788. The method of item 11497 wherein the combining is performed by spraying the implant into a solution that comprises the agent, a polymer and a solvent that will dissolve the implant.

11789. A method of making a medical device comprising: combining a central venous catheter implant and an anti-scarring agent or a composition comprising an anti-scarring agent, wherein the agent inhibits scarring between the device and a host into which the device is implanted.

11790. The method of item 11789 wherein the agent inhibits cell regeneration.

11791. The method of item 11789 wherein the agent inhibits angiogenesis.

11792. The method of item 11789 wherein the agent inhibits fibroblast migration.

11793. The method of item 11789 wherein the agent inhibits fibroblast proliferation.

11794. The method of item 11789 wherein the agent inhibits deposition of extracellular matrix.

11795. The method of item 11789 wherein the agent inhibits tissue remodeling.

11796. The method of item 11789 wherein the agent is an angiogenesis inhibitor.

11797. The method of item 11789 wherein the agent is a 5-lipoxygenase inhibitor or antagonist.

11798. The method of item 11789 wherein the agent is a chemokine receptor antagonist.

11799. The method of item 11789 wherein the agent is a cell cycle inhibitor.

11800. The method of item 11789 wherein the agent is a taxane.

11801. The method of item 11789 wherein the agent is an anti-microtubule agent.

11802. The method of item 11789 wherein the agent is paclitaxel.

11803. The method of item 11789 wherein the agent is not paclitaxel.

11804. The method of item 11789 wherein the agent is an analogue or derivative of paclitaxel.

11805. The method of item 11789 wherein the agent is a vinca alkaloid.

11806. The method of item 11789 wherein the agent is camptothecin or an analogue or derivative thereof.

11807. The method of item 11789 wherein the agent is a podophyllotoxin.

11808. The method of item 11789 wherein the agent is a podophyllotoxin, wherein the podophyllotoxin is etoposide or an analogue or derivative thereof.

11809. The method of item 11789 wherein the agent is an anthracycline.

11810. The method of item 11789 wherein the agent is an anthracycline, wherein the anthracycline is doxorubicin or an analogue or derivative thereof.

11811. The method of item 11789 wherein the agent is an anthracycline, wherein the anthracycline is mitoxantrone or an analogue or derivative thereof.

11812. The method of item 11789 wherein the agent is a platinum compound.

11813. The method of item 11789 wherein the agent is a nitrosourea.

11814. The method of item 11789 wherein the agent is a nitroimidazole.

11815. The method of item 11789 wherein the agent is a folic acid antagonist.

11816. The method of item 11789 wherein the agent is a cytidine analogue.

11817. The method of item 11789 wherein the agent is a pyrimidine analogue.

11818. The method of item 11789 wherein the agent is a fluoropyrimidine analogue.

11819. The method of item 11789 wherein the agent is a purine analogue.

11820. The method of item 11789 wherein the agent is a nitrogen mustard or an analogue or derivative thereof.

11821. The method of item 11789 wherein the agent is a hydroxyurea.

11822. The method of item 11789 wherein the agent is a mytomicin or an analogue or derivative thereof.

11823. The method of item 11789 wherein the agent is an alkyl sulfonate.

11824. The method of item 11789 wherein the agent is a benzamide or an analogue or derivative thereof.

11825. The method of item 11789 wherein the agent is a nicotinamide or an analogue or derivative thereof.

11826. The method of item 11789 wherein the agent is a halogenated sugar or an analogue or derivative thereof.

11827. The method of item 11789 wherein the agent is a DNA alkylating agent.

11828. The method of item 11789 wherein the agent is an anti-microtubule agent.

11829. The method of item 11789 wherein the agent is a topoisomerase inhibitor.

11830. The method of item 11789 wherein the agent is a DNA cleaving agent.

11831. The method of item 11789 wherein the agent is an antimetabolite.

11832. The method of item 11789 wherein the agent inhibits adenosine deaminase.

11833. The method of item 11789 wherein the agent inhibits purine ring synthesis.

11834. The method of item 11789 wherein the agent is a nucleotide interconversion inhibitor.

11835. The method of item 11789 wherein the agent inhibits dihydrofolate reduction.

11836. The method of item 11789 wherein the agent blocks thymidine monophosphate.

11837. The method of item 11789 wherein the agent causes DNA damage.

11838. The method of item 11789 wherein the agent is a DNA intercalation agent.

11839. The method of item 11789 wherein the agent is a RNA synthesis inhibitor.

11840. The method of item 11789 wherein the agent is a pyrimidine synthesis inhibitor.

11841. The method of item 11789 wherein the agent inhibits ribonucleotide synthesis or function.

11842. The method of item 11789 wherein the agent inhibits thymidine monophosphate synthesis or function.

11843. The method of item 11789 wherein the agent inhibits DNA synthesis.

11844. The method of item 11789 wherein the agent causes DNA adduct formation.

11845. The method of item 11789 wherein the agent inhibits protein synthesis.

11846. The method of item 11789 wherein the agent inhibits microtubule function.

11847. The method of item 11789 wherein the agent is a cyclin dependent protein kinase inhibitor.

11848. The method of item 11789 wherein the agent is an epidermal growth factor kinase inhibitor.

11849. The method of item 11789 wherein the agent is an elastase inhibitor.

11850. The method of item 11789 wherein the agent is a factor Xa inhibitor.

11851. The method of item 11789 wherein the agent is a farnesyltransferase inhibitor.

11852. The method of item 11789 wherein the agent is a fibrinogen antagonist.

11853. The method of item 11789 wherein the agent is a guanylate cyclase stimulant.

11854. The method of item 11789 wherein the agent is a heat shock protein 90 antagonist.

11855. The method of item 11789 wherein the agent is a heat shock protein 90 antagonist, wherein the heat shock protein 90 antagonist is geldanamycin or an analogue or derivative thereof.

11856. The method of item 11789 wherein the agent is a guanylate cyclase stimulant.

11857. The method of item 11789 wherein the agent is a HMGCoA reductase inhibitor.

11858. The method of item 11789 wherein the agent is a HMGCoA reductase inhibitor, wherein the HMGCoA reductase inhibitor is simvastatin or an analogue or derivative thereof.

11859. The method of item 11789 wherein the agent is a hydroorotate dehydrogenase inhibitor.

11860. The method of item 11789 wherein the agent is an IKK2 inhibitor.

11861. The method of item 11789 wherein the agent is an IL-1 antagonist.

11862. The method of item 11789 wherein the agent is an ICE antagonist.

11863. The method of item 11789 wherein the agent is an IRAK antagonist.

11864. The method of item 11789 wherein the agent is an IL-4 agonist.

11865. The method of item 11789 wherein the agent is an immunomodulatory agent.

11866. The method of item 11789 wherein the agent is sirolimus or an analogue or derivative thereof.

11867. The method of item 11789 wherein the agent is not sirolimus.

11868. The method of item 11789 wherein the agent is everolimus or an analogue or derivative thereof.

11869. The method of item 11789 wherein the agent is tacrolimus or an analogue or derivative thereof.

11870. The method of item 11789 wherein the agent is not tacrolimus.

11871. The method of item 11789 wherein the agent is biolmus or an analogue or derivative thereof.

11872. The method of item 11789 wherein the agent is tresperimus or an analogue or derivative thereof.

11873. The method of item 11789 wherein the agent is auranofin or an analogue or derivative thereof.

11874. The method of item 11789 wherein the agent is 27-0-demethylrapamycin or an analogue or derivative thereof.

11875. The method of item 11789 wherein the agent is gusperimus or an analogue or derivative thereof.

11876. The method of item 11789 wherein the agent is pimecrolimus or an analogue or derivative thereof.

11877. The method of item 11789 wherein the agent is ABT-578 or an analogue or derivative thereof.

11878. The method of item 11789 wherein the agent is an inosine monophosphate dehydrogenase (IMPDH) inhibitor.

11879. The method of item 11789 wherein the agent is an IMPDH inhibitor, wherein the IMPDH inhibitor is mycophenolic acid or an analogue or derivative thereof.

11880. The method of item 11789 wherein the agent is an IMPDH inhibitor, wherein the IMPDH inhibitor is 1-alpha-25 dihydroxy vitamin D₃ or an analogue or derivative thereof.

11881. The method of item 11789 wherein the agent is a leukotriene inhibitor.

11882. The method of item 11789 wherein the agent is a MCP-1 antagonist.

11883. The method of item 11789 wherein the agent is a MMP inhibitor.

11884. The method of item 11789 wherein the agent is an NF kappa B inhibitor.

11885. The method of item 11789 wherein the agent is an NF kappa B inhibitor, wherein the NF kappa B inhibitor is Bay 11-7082.

11886. The method of item 11789 wherein the agent is an NO agonist.

11887. The method of item 11789 wherein the agent is a p38 MAP kinase inhibitor.

11888. The method of item 11789 wherein the agent is a p38 MAP kinase inhibitor, wherein the p38 MAP kinase inhibitor is SB 202190.

11889. The method of item 11789 wherein the agent is a phosphodiesterase inhibitor.

11890. The method of item 11789 wherein the agent is a TGF beta inhibitor.

11891. The method of item 11789 wherein the agent is a thromboxane A2 antagonist.

11892. The method of item 11789 wherein the agent is a TNFa antagonist.

11893. The method of item 11789 wherein the agent is a TACE inhibitor.

11894. The method of item 11789 wherein the agent is a tyrosine kinase inhibitor.

11895. The method of item 11789 wherein the agent is a vitronectin inhibitor.

11896. The method of item 11789 wherein the agent is a fibroblast growth factor inhibitor.

11897. The method of item 11789 wherein the agent is a protein kinase inhibitor.

11898. The method of item 11789 wherein the agent is a PDGF receptor kinase inhibitor.

11899. The method of item 11789 wherein the agent is an endothelial growth factor receptor kinase inhibitor.

11900. The method of item 11789 wherein the agent is a retinoic acid receptor antagonist.

11901. The method of item 11789 wherein the agent is a platelet derived growth factor receptor kinase inhibitor.

11902. The method of item 11789 wherein the agent is a fibronogin antagonist.

11903. The method of item 11789 wherein the agent is an antimycotic agent.

11904. The method of item 11789 wherein the agent is an antimycotic agent, wherein the antimycotic agent is sulconizole.

11905. The method of item 11789 wherein the agent is a bisphosphonate.

11906. The method of item 11789 wherein the agent is a phospholipase A1 inhibitor.

11907. The method of item 11789 wherein the agent is a histamine H1/H2/H3 receptor antagonist.

11908. The method of item 11789 wherein the agent is a macrolide antibiotic.

11909. The method of item 11789 wherein the agent is a GPIIb/IIIa receptor antagonist.

11910. The method of item 11789 wherein the agent is an endothelin receptor antagonist.

11911. The method of item 11789 wherein the agent is a peroxisome proliferator-activated receptor agonist.

11912. The method of item 11789 wherein the agent is an estrogen receptor agent.

11913. The method of item 11789 wherein the agent is a somastostatin analogue.

11914. The method of item 11789 wherein the agent is a neurokinin 1 antagonist.

11915. The method of item 11789 wherein the agent is a neurokinin 3 antagonist.

11916. The method of item 11789 wherein the agent is a VLA-4 antagonist.

11917. The method of item 11789 wherein the agent is an osteoclast inhibitor.

11918. The method of item 11789 wherein the agent is a DNA topoisomerase ATP hydrolyzing inhibitor.

11919. The method of item 11789 wherein the agent is an angiotensin I converting enzyme inhibitor.

11920. The method of item 11789 wherein the agent is an angiotensin II antagonist.

11921. The method of item 11789 wherein the agent is an enkephalinase inhibitor.

11922. The method of item 11789 wherein the agent is a peroxisome proliferator-activated receptor gamma agonist insulin sensitizer.

11923. The method of item 11789 wherein the agent is a protein kinase C inhibitor.

11924. The method of item 11789 wherein the agent is a ROCK (rho-associated kinase) inhibitor.

11925. The method of item 11789 wherein the agent is a CXCR3 inhibitor.

11926. The method of item 11789 wherein the agent is an Itk inhibitor.

11927. The method of item 11789 wherein the agent is a cytosolic phospholipase A₂-alpha inhibitor.

11928. The method of item 11789 wherein the agent is a PPAR agonist.

11929. The method of item 11789 wherein the agent is an immunosuppressant.

11930. The method of item 11789 wherein the agent is an Erb inhibitor.

11931. The method of item 11789 wherein the agent is an apoptosis agonist.

11932. The method of item 11789 wherein the agent is a lipocortin agonist.

11933. The method of item 11789 wherein the agent is a VCAM-1 antagonist.

11934. The method of item 11789 wherein the agent is a collagen antagonist.

11935. The method of item 11789 wherein the agent is an alpha 2 integrin antagonist.

11936. The method of item 11789 wherein the agent is a TNF alpha inhibitor.

11937. The method of item 11789 wherein the agent is a nitric oxide inhibitor.

11938. The method of item 11789 wherein the agent is a cathepsin inhibitor.

11939. The method of item 11789 wherein the agent is not an anti-inflammatory agent.

11940. The method of item 11789 wherein the agent is not a steroid.

11941. The method of item 11789 wherein the agent is not a glucocorticosteroid.

11942. The method of item 11789 wherein the agent is not dexamethasone.

11943. The method of item 11789 wherein the agent is not an anti-infective agent.

11944. The method of item 11789 wherein the agent is not an antibiotic.

11945. The method of item 11789 wherein the agent is not an anti-fungal agent.

11946. The method of item 11789, wherein the composition comprises a polymer.

11947. The method of item 11789, wherein the composition comprises a polymeric carrier.

11948. The method of item 11789 wherein the anti-scarring agent inhibits adhesion between the device and a host into which the device is implanted.

11949. The method of item 11789 wherein the device delivers the anti-scarring agent locally to tissue proximate to the device.

11950. The method of item 11789 wherein the device has a coating that comprises the anti-scarring agent.

11951. The method of item 11789, wherein the device has a coating that comprises the agent and is disposed on a surface of the implant.

11952. The method of item 11789, wherein the device has a coating that comprises the agent and directly contacts the implant.

11953. The method of item 11789, wherein the device has a coating that comprises the agent and indirectly contacts the implant.

11954. The method of item 11789, wherein the device has a coating that comprises the agent and partially covers the implant.

11955. The method of item 11789, wherein the device has a coating that comprises the agent and completely covers the implant.

11956. The method of item 11789, wherein the device has a uniform coating.

11957. The method of item 11789, wherein the device has a non-uniform coating.

11958. The method of item 11789, wherein the device has a discontinuous coating.

11959. The method of item 11789, wherein the device has a patterned coating.

11960. The method of item 11789, wherein the device has a coating with a thickness of 100 μm or less.

11961. The method of item 11789, wherein the device has a coating with a thickness of 10 μm or less.

11962. The method of item 11789, wherein the device has a coating, and the coating adheres to the surface of the implant upon deployment of the implant.

11963. The method of item 11789, wherein the device has a coating, and wherein the coating is stable at room temperature for a period of 1 year.

11964. The method of item 11789, wherein the device has a coating, and wherein the anti-scarring agent is present in the coating in an amount ranging between about 0.0001% to about 1% by weight.

11965. The method of item 11789, wherein the device has a coating, and wherein the anti-scarring agent is present in the coating in an amount ranging between about 1% to about 10% by weight.

11966. The method of item 11789, wherein the device has a coating, and wherein the anti-scarring agent is present in the coating in an amount ranging between about 10% to about 25% by weight.

11967. The method of item 11789, wherein the device has a coating, and wherein the anti-scarring agent is present in the coating in an amount ranging between about 25% to about 70% by weight.

11968. The method of item 11789, wherein the device has a coating, and wherein the coating further comprises a polymer.

11969. The method of item 11789, wherein the device has a first coating having a first composition and a second coating having a second composition.

11970. The method of item 11789, wherein the device has a first coating having a first composition and a second coating having a second composition, wherein the first composition and the second composition are different.

11971. The method of item 11789, wherein the composition comprises a polymer.

11972. The method of item 11789, wherein the composition comprises a polymeric carrier.

11973. The method of item 11789, wherein the composition comprises a polymeric carrier, and wherein the polymeric carrier comprises a copolymer.

11974. The method of item 11789, wherein the composition comprises a polymeric carrier, and wherein the polymeric carrier comprises a block copolymer.

11975. The method of item 11789, wherein the composition comprises a polymeric carrier, and wherein the polymeric carrier comprises a random copolymer.

11976. The method of item 11789, wherein the composition comprises a polymeric carrier, and wherein the polymeric carrier comprises a biodegradable polymer.

11977. The method of item 11789, wherein the composition comprises a polymeric carrier, and wherein the polymeric carrier comprises a non-biodegradable polymer.

11978. The method of item 11789, wherein the composition comprises a polymeric carrier, and wherein the polymeric carrier comprises a hydrophilic polymer.

11979. The method of item 11789, wherein the composition comprises a polymeric carrier, and wherein the polymeric carrier comprises a hydrophobic polymer.

11980. The method of item 11789, wherein the composition comprises a polymeric carrier, and wherein the polymeric carrier comprises a polymer having hydrophilic domains.

11981. The method of item 11789, wherein the composition comprises a polymeric carrier, and wherein the polymeric carrier comprises a polymer having hydrophobic domains.

11982. The method of item 11789, wherein the composition comprises a polymeric carrier, and wherein the polymeric carrier comprises a non-conductive polymer.

11983. The method of item 11789, wherein the composition comprises a polymeric carrier, and wherein the polymeric carrier comprises an elastomer.

11984. The method of item 11789, wherein the composition comprises a polymeric carrier, and wherein the polymeric carrier comprises a hydrogel.

11985. The method of item 11789, wherein the composition comprises a polymeric carrier, and wherein the polymeric carrier comprises a silicone polymer.

11986. The method of item 11789, wherein the composition comprises a polymeric carrier, and wherein the polymeric carrier comprises a hydrocarbon polymer.

11987. The method of item 11789, wherein the composition comprises a polymeric carrier, and wherein the polymeric carrier comprises a styrene-derived polymer.

11988. The method of item 11789, wherein the composition comprises a polymeric carrier, and wherein the polymeric carrier comprises a butadiene polymer.

11989. The method of item 11789, wherein the composition comprises a polymeric carrier, and wherein the polymeric carrier comprises a macromer.

11990. The method of item 11789, wherein the composition comprises a polymeric carrier, and wherein the polymeric carrier comprises a poly(ethylene glycol)polymer.

11991. The method of item 11789 wherein the composition comprises a polymeric carrier, and wherein the polymeric carrier comprises an amorphous polymer.

11992. The method of item 11789, wherein the device comprises a lubricious coating.

11993. The method of item 11789 wherein the anti-scarring agent is located within pores or holes of the device.

11994. The method of item 11789 wherein the anti-scarring agent is located within a channel, lumen, or divet of the device.

11995. The method of item 11789, wherein the device comprises a second pharmaceutically active agent.

11996. The method of item 11789 wherein the device comprises an anti-inflammatory agent.

11997. The method of item 11789 wherein the device comprises an agent that inhibits infection.

11998. The method of item 11789 wherein the device comprises an agent that inhibits infection, and wherein the agent is an anthracycline.

11999. The method of item 11789 wherein the device comprises an agent that inhibits infection, and wherein the agent is doxorubicin.

12000. The method of item 11789 wherein the device comprises an agent that inhibits infection, and wherein the agent is mitoxantrone.

12001. The method of item 11789 wherein the device comprises an agent that inhibits infection, and wherein the agent is a fluoropyrimidine.

12002. The method of item 11789 wherein the device comprises an agent that inhibits infection, and wherein the agent is 5-fluorouracil (5-FU).

12003. The method of item 11789 wherein the device comprises an agent that inhibits infection, and wherein the agent is a folic acid antagonist.

12004. The method of item 11789 wherein the device comprises an agent that inhibits infection, and wherein the agent is methotrexate.

12005. The method of item 11789 wherein the device comprises an agent that inhibits infection, and wherein the agent is a podophylotoxin.

12006. The method of item 11789 wherein the device comprises an agent that inhibits infection, and wherein the agent is etoposide.

12007. The method of item 11789 wherein the device comprises an agent that inhibits infection, and wherein the agent is a camptothecin.

12008. The method of item 11789 wherein the device comprises an agent that inhibits infection, and wherein the agent is a hydroxyurea.

12009. The method of item 11789 wherein the device comprises an agent that inhibits infection, and wherein the agent is a platinum complex.

12010. The method of item 11789 wherein the device comprises an agent that inhibits infection, and wherein the agent is cisplatin.

12011. The method of item 11789, further comprising an anti-thrombotic agent.

12012. The method of item 11789 wherein the device comprises a visualization agent.

12013. The method of item 11789 wherein the device comprises a visualization agent, wherein the visualization agent is a radiopaque material, and wherein the radiopaque material comprises a metal, a halogenated compound, or a barium containing compound.

12014. The method of item 11789 wherein the device comprises a visualization agent, wherein the visualization agent is a radiopaque material, and wherein the radiopaque material comprises barium, tantalum, or technetium.

12015. The method of item 11789 wherein the device comprises a visualization agent, and wherein the visualization agent is a MRI responsive material.

12016. The method of item 11789 wherein the device comprises a visualization agent, and wherein the visualization agent comprises a gadolinium chelate.

12017. The method of item 11789 wherein the device comprises a visualization agent, and wherein the visualization agent comprises iron, magnesium, manganese, copper, or chromium.

12018. The method of item 11789 wherein the device comprises a visualization agent, and wherein the visualization agent comprises an iron oxide compound.

12019. The method of item 11789 wherein the device comprises a visualization agent, and wherein the visualization agent comprises a dye, pigment, or colorant.

12020. The method of item 11789 wherein the device comprises an echogenic material.

12021. The method of item 11789 wherein the device comprises an echogenic material, and wherein the echogenic material is in the form of a coating.

12022. The method of item 11789 wherein the device is sterile.

12023. The method of item 11789 wherein the anti-scarring agent is released into tissue in the vicinity of the device after deployment of the device.

12024. The method of item 11789 wherein the anti-scarring agent is released into tissue in the vicinity of the device after deployment of the device, and wherein the tissue is connective tissue.

12025. The method of item 11789 wherein the anti-scarring agent is released into tissue in the vicinity of the device after deployment of the device, and wherein the tissue is muscle tissue.

12026. The method of item 11789 wherein the anti-scarring agent is released into tissue in the vicinity of the device after deployment of the device, and wherein the tissue is nerve tissue.

12027. The method of item 11789 wherein the anti-scarring agent is released into tissue in the vicinity of the device after deployment of the device, and wherein the tissue is epithelium tissue.

12028. The method of item 11789 wherein the anti-scarring agent is released in effective concentrations from the device over a period ranging from the time of deployment of the device to about 1 year.

12029. The method of item 11789 wherein the anti-scarring agent is released in effective concentrations from the device over a period ranging from about 1 month to 6 months.

12030. The method of item 11789 wherein the anti-scarring agent is released in effective concentrations from the device over a period ranging from about 1-90 days.

12031. The method of item 11789 wherein the anti-scarring agent is released in effective concentrations from the device at a constant rate.

12032. The method of item 11789 wherein the anti-scarring agent is released in effective concentrations from the device at an increasing rate.

12033. The method of item 11789 wherein the anti-scarring agent is released in effective concentrations from the device at a decreasing rate.

12034. The method of item 11789 wherein the anti-scarring agent is released in effective concentrations from the composition comprising the anti-scarring agent by diffusion over a period ranging from the time of deployment of the device to about 90 days.

12035. The method of item 11789 wherein the anti-scarring agent is released in effective concentrations from the composition comprising the anti-scarring agent by erosion of the composition over a period ranging from the time of deployment of the device to about 90 days.

12036. The method of item 11789 wherein the device comprises about 0.01 μg to about 10 μg of the anti-scarring agent.

12037. The method of item 11789 wherein the device comprises about 10 μg to about 10 mg of the anti-scarring agent.

12038. The method of item 11789 wherein the device comprises about 10 mg to about 250 mg of the anti-scarring agent.

12039. The method of item 11789 wherein the device comprises about 250 mg to about 1000 mg of the anti-scarring agent.

12040. The method of item 11789 wherein the device comprises about 1000 mg to about 2500 mg of the anti-scarring agent.

12041. The method of item 11789 wherein a surface of the device comprises less than 0.01 μg of the anti-scarring agent per mm² of device surface to which the anti-scarring agent is applied.

12042. The method of item 11789 wherein a surface of the device comprises about 0.01 μg to about 1 μg of the anti-scarring agent per mm² of device surface to which the anti-scarring agent is applied.

12043. The method of item 11789 wherein a surface of the device comprises about 1 μg to about 10 μg of the anti-scarring agent per mm² of device surface to which the anti-scarring agent is applied.

12044. The method of item 11789 wherein a surface of the device comprises about 10 μg to about 250 μg of the anti-scarring agent per mm² of device surface to which the anti-scarring agent is applied.

12045. The method of item 11789 wherein a surface of the device comprises about 250 μg to about 1000 μg of the anti-scarring agent of anti-scarring agent per mm² of device surface to which the anti-scarring agent is applied.

12046. The method of item 11789 wherein a surface of the device comprises about 1000 μg to about 2500 μg of the anti-scarring agent per mm² of device surface to which the anti-scarring agent is applied.

12047. The method of item 11789 wherein the combining is performed by direct affixing the agent or the composition to the implant.

12048. The method of item 11789 wherein the combining is performed by spraying the agent or the component onto the implant.

12049. The method of item 11789 wherein the combining is performed by electrospraying the agent or the composition onto the implant.

12050. The method of item 11789 wherein the combining is performed by dipping the implant into a solution comprising the agent or the composition.

12051. The method of item 11789 wherein the combining is performed by covalently attaching the agent or the composition to the implant.

12052. The method of item 11789 wherein the combining is performed by non-covalently attaching the agent or the composition to the implant.

12053. The method of item 11789 wherein the combining is performed by coating the implant with a substance that contains the agent or the composition.

12054. The method of item 11789 wherein the combining is performed by coating the implant with a substance that absorbs the agent.

12055. The method of item 11789 wherein the combining is performed by interweaving a thread composed of, or coated with, the agent or the composition.

12056. The method of item 11789 wherein the combining is performed by covering all the implant with a sleeve that contains the agent or the composition.

12057. The method of item 11789 wherein the combining is performed by covering a portion of the implant with a sleeve that contains the agent or the composition.

12058. The method of item 11789 wherein the combining is performed by covering all the implant with a cover that contains the agent or the composition.

12059. The method of item 11789 wherein the combining is performed by covering a portion of the implant with a cover that contains the agent or the composition.

12060. The method of item 11789 wherein the combining is performed by covering all the implant with an electrospun fabric that contains the agent or the composition.

12061. The method of item 11789 wherein the combining is performed by covering a portion of the implant with an electrospun fabric that contains the agent or the composition.

12062. The method of item 11789 wherein the combining is performed by covering all the implant with a mesh that contains the agent or the composition.

12063. The method of item 11789 wherein the combining is performed by covering a portion of the implant with a mesh that contains the agent or the composition.

12064. The method of item 11789 wherein the combining is performed by constructing all the implant with the agent or the composition.

12065. The method of item 11789 wherein the combining is performed by constructing a portion of the implant with the agent or the composition.

12066. The method of item 11789 wherein the combining is performed by impregnating the implant with the agent or the composition.

12067. The method of item 11789 wherein the combining is performed by constructing all of the implant from a degradable polymer that releases the agent.

12068. The method of item 11789 wherein the combining is performed by constructing a portion of the implant from a degradable polymer that releases the agent.

12069. The method of item 11789 wherein the combining is performed by dipping the implant into a solution that comprise the agent and an inert solvent for the implant.

12070. The method of item 11789 wherein the combining is performed by dipping the implant into a solution that comprises the agent and a solvent that will swill the implant.

12071. The method of item 11789 wherein the combining is performed by dipping the implant into a solution that comprises the agent and a solvent that will dissolve the implant.

12072. The method of item 11789 wherein the combining is performed by dipping the implant into a solution that comprises the agent, a polymer and an inert solvent for the implant.

12073. The method of item 11789 wherein the combining is performed by dipping the implant into a solution that comprises the agent, a polymer and a solvent that will swill the implant.

12074. The method of item 11789 wherein the combining is performed by dipping the implant into a solution that comprises the agent, a polymer and a solvent that will dissolve the implant.

12075. The method of item 11789 wherein the combining is performed by spraying the implant into a solution that comprises the agent and an inert solvent for the implant.

12076. The method of item 11789 wherein the combining is performed by spraying the implant into a solution that comprises the agent and a solvent that will swill the implant.

12077. The method of item 11789 wherein the combining is performed by spraying the implant into a solution that comprises the agent and a solvent that will dissolve the implant.

12078. The method of item 11789 wherein the combining is performed by spraying the implant into a solution that comprises the agent, a polymer and an inert solvent for the implant.

12079. The method of item 11789 wherein the combining is performed by spraying the implant into a solution that comprises the agent, a polymer and a solvent that will swill the implant.

12080. The method of item 11789 wherein the combining is performed by spraying the implant into a solution that comprises the agent, a polymer and a solvent that will dissolve the implant.

12081. The method of item 11789 wherein the implant is a total parenteral nutrition catheter.

12082. The method of item 11789 wherein the implant is a flow-directed balloon-tipped pulmonary artery catheter.

12083. A method of making a medical device comprising: combining a prosthetic heart valve implant and an anti-scarring agent or a composition comprising an anti-scarring agent, wherein the agent inhibits scarring between the device and a host into which the device is implanted.

12084. The method of item 12083 wherein the agent inhibits cell regeneration.

12085. The method of item 12083 wherein the agent inhibits angiogenesis.

12086. The method of item 12083 wherein the agent inhibits fibroblast migration.

12087. The method of item 12083 wherein the agent inhibits fibroblast proliferation.

12088. The method of item 12083 wherein the agent inhibits deposition of extracellular matrix.

12089. The method of item 12083 wherein the agent inhibits tissue remodeling.

12090. The method of item 12083 wherein the agent is an angiogenesis inhibitor.

12091. The method of item 12083 wherein the agent is a 5-lipoxygenase inhibitor or antagonist.

12092. The method of item 12083 wherein the agent is a chemokine receptor antagonist.

12093. The method of item 12083 wherein the agent is a cell cycle inhibitor.

12094. The method of item 12083 wherein the agent is a taxane.

12095. The method of item 12083 wherein the agent is an anti-microtubule agent.

12096. The method of item 12083 wherein the agent is paclitaxel.

12097. The method of item 12083 wherein the agent is not paclitaxel.

12098. The method of item 12083 wherein the agent is an analogue or derivative of paclitaxel.

12099. The method of item 12083 wherein the agent is a vinca alkaloid.

12100. The method of item 12083 wherein the agent is camptothecin or an analogue or derivative thereof.

12101. The method of item 12083 wherein the agent is a podophyllotoxin.

12102. The method of item 12083 wherein the agent is a podophyllotoxin, wherein the podophyllotoxin is etoposide or an analogue or derivative thereof.

12103. The method of item 12083 wherein the agent is an anthracycline.

12104. The method of item 12083 wherein the agent is an anthracycline, wherein the anthracycline is doxorubicin or an analogue or derivative thereof.

12105. The method of item 12083 wherein the agent is an anthracycline, wherein the anthracycline is mitoxantrone or an analogue or derivative thereof.

12106. The method of item 12083 wherein the agent is a platinum compound.

12107. The method of item 12083 wherein the agent is a nitrosourea.

12108. The method of item 12083 wherein the agent is a nitroimidazole.

12109. The method of item 12083 wherein the agent is a folic acid antagonist.

12110. The method of item 12083 wherein the agent is a cytidine analogue.

12111. The method of item 12083 wherein the agent is a pyrimidine analogue.

12112. The method of item 12083 wherein the agent is a fluoropyrimidine analogue.

12113. The method of item 12083 wherein the agent is a purine analogue.

12114. The method of item 12083 wherein the agent is a nitrogen mustard or an analogue or derivative thereof.

12115. The method of item 12083 wherein the agent is a hydroxyurea.

12116. The method of item 12083 wherein the agent is a mytomicin or an analogue or derivative thereof.

12117. The method of item 12083 wherein the agent is an alkyl sulfonate.

12118. The method of item 12083 wherein the agent is a benzamide or an analogue or derivative thereof.

12119. The method of item 12083 wherein the agent is a nicotinamide or an analogue or derivative thereof.

12120. The method of item 12083 wherein the agent is a halogenated sugar or an analogue or derivative thereof.

12121. The method of item 12083 wherein the agent is a DNA alkylating agent.

12122. The method of item 12083 wherein the agent is an anti-microtubule agent.

12123. The method of item 12083 wherein the agent is a topoisomerase inhibitor.

12124. The method of item 12083 wherein the agent is a DNA cleaving agent.

12125. The method of item 12083 wherein the agent is an antimetabolite.

12126. The method of item 12083 wherein the agent inhibits adenosine deaminase.

12127. The method of item 12083 wherein the agent inhibits purine ring synthesis.

12128. The method of item 12083 wherein the agent is a nucleotide interconversion inhibitor.

12129. The method of item 12083 wherein the agent inhibits dihydrofolate reduction.

12130. The method of item 12083 wherein the agent blocks thymidine monophosphate.

12131. The method of item 12083 wherein the agent causes DNA damage.

12132. The method of item 12083 wherein the agent is a DNA intercalation agent.

12133. The method of item 12083 wherein the agent is a RNA synthesis inhibitor.

12134. The method of item 12083 wherein the agent is a pyrimidine synthesis inhibitor.

12135. The method of item 12083 wherein the agent inhibits ribonucleotide synthesis or function.

12136. The method of item 12083 wherein the agent inhibits thymidine monophosphate synthesis or function.

12137. The method of item 12083 wherein the agent inhibits DNA synthesis.

12138. The method of item 12083 wherein the agent causes DNA adduct formation.

12139. The method of item 12083 wherein the agent inhibits protein synthesis.

12140. The method of item 12083 wherein the agent inhibits microtubule function.

12141. The method of item 12083 wherein the agent is a cyclin dependent protein kinase inhibitor.

12142. The method of item 12083 wherein the agent is an epidermal growth factor kinase inhibitor.

12143. The method of item 12083 wherein the agent is an elastase inhibitor.

12144. The method of item 12083 wherein the agent is a factor Xa inhibitor.

12145. The method of item 12083 wherein the agent is a farnesyltransferase inhibitor.

12146. The method of item 12083 wherein the agent is a fibrinogen antagonist.

12147. The method of item 12083 wherein the agent is a guanylate cyclase stimulant.

12148. The method of item 12083 wherein the agent is a heat shock protein 90 antagonist.

12149. The method of item 12083 wherein the agent is a heat shock protein 90 antagonist, wherein the heat shock protein 90 antagonist is geldanamycin or an analogue or derivative thereof.

12150. The method of item 12083 wherein the agent is a guanylate cyclase stimulant.

12151. The method of item 12083 wherein the agent is a HMGCoA reductase inhibitor.

12152. The method of item 12083 wherein the agent is a HMGCoA reductase inhibitor, wherein the HMGCoA reductase inhibitor is simvastatin or an analogue or derivative thereof.

12153. The method of item 12083 wherein the agent is a hydroorotate dehydrogenase inhibitor.

12154. The method of item 12083 wherein the agent is an IKK2 inhibitor.

12155. The method of item 12083 wherein the agent is an IL-1 antagonist.

12156. The method of item 12083 wherein the agent is an ICE antagonist.

12157. The method of item 12083 wherein the agent is an IRAK antagonist.

12158. The method of item 12083 wherein the agent is an IL-4 agonist.

12159. The method of item 12083 wherein the agent is an immunomodulatory agent.

12160. The method of item 12083 wherein the agent is sirolimus or an analogue or derivative thereof.

12161. The method of item 12083 wherein the agent is not sirolimus.

12162. The method of item 12083 wherein the agent is everolimus or an analogue or derivative thereof.

12163. The method of item 12083 wherein the agent is tacrolimus or an analogue or derivative thereof.

12164. The method of item 12083 wherein the agent is not tacrolimus.

12165. The method of item 12083 wherein the agent is biolmus or an analogue or derivative thereof.

12166. The method of item 12083 wherein the agent is tresperimus or an analogue or derivative thereof.

12167. The method of item 12083 wherein the agent is auranofin or an analogue or derivative thereof.

12168. The method of item 12083 wherein the agent is 27-0-demethylrapamycin or an analogue or derivative thereof.

12169. The method of item 12083 wherein the agent is gusperimus or an analogue or derivative thereof.

12170. The method of item 12083 wherein the agent is pimecrolimus or an analogue or derivative thereof.

12171. The method of item 12083 wherein the agent is ABT-578 or an analogue or derivative thereof.

12172. The method of item 12083 wherein the agent is an inosine monophosphate dehydrogenase (IMPDH) inhibitor.

12173. The method of item 12083 wherein the agent is an IMPDH inhibitor, wherein the IMPDH inhibitor is mycophenolic acid or an analogue or derivative thereof.

12174. The method of item 12083 wherein the agent is an IMPDH inhibitor, wherein the IMPDH inhibitor is 1-alpha-25 dihydroxy vitamin D₃ or an analogue or derivative thereof.

12175. The method of item 12083 wherein the agent is a leukotriene inhibitor.

12176. The method of item 12083 wherein the agent is a MCP-1 antagonist.

12177. The method of item 12083 wherein the agent is a MMP inhibitor.

12178. The method of item 12083 wherein the agent is an NF kappa B inhibitor.

12179. The method of item 12083 wherein the agent is an NF kappa B inhibitor, wherein the NF kappa B inhibitor is Bay 11-7082.

12180. The method of item 12083 wherein the agent is an NO agonist.

12181. The method of item 12083 wherein the agent is a p38 MAP kinase inhibitor.

12182. The method of item 12083 wherein the agent is a p38 MAP kinase inhibitor, wherein the p38 MAP kinase inhibitor is SB 202190.

12183. The method of item 12083 wherein the agent is a phosphodiesterase inhibitor.

12184. The method of item 12083 wherein the agent is a TGF beta inhibitor.

12185. The method of item 12083 wherein the agent is a thromboxane A2 antagonist.

12186. The method of item 12083 wherein the agent is a TNFa antagonist.

12187. The method of item 12083 wherein the agent is a TACE inhibitor.

12188. The method of item 12083 wherein the agent is a tyrosine kinase inhibitor.

12189. The method of item 12083 wherein the agent is a vitronectin inhibitor.

12190. The method of item 12083 wherein the agent is a fibroblast growth factor inhibitor.

12191. The method of item 12083 wherein the agent is a protein kinase inhibitor.

12192. The method of item 12083 wherein the agent is a PDGF receptor kinase inhibitor.

12193. The method of item 12083 wherein the agent is an endothelial growth factor receptor kinase inhibitor.

12194. The method of item 12083 wherein the agent is a retinoic acid receptor antagonist.

12195. The method of item 12083 wherein the agent is a platelet derived growth factor receptor kinase inhibitor.

12196. The method of item 12083 wherein the agent is a fibronogin antagonist.

12197. The method of item 12083 wherein the agent is an antimycotic agent.

12198. The method of item 12083 wherein the agent is an antimycotic agent, wherein the antimycotic agent is sulconizole.

12199. The method of item 12083 wherein the agent is a bisphosphonate.

12200. The method of item 12083 wherein the agent is a phospholipase A1 inhibitor.

12201. The method of item 12083 wherein the agent is a histamine H1/H2/H3 receptor antagonist.

12202. The method of item 12083 wherein the agent is a macrolide antibiotic.

12203. The method of item 12083 wherein the agent is a GPIIb/IIIa receptor antagonist.

12204. The method of item 12083 wherein the agent is an endothelin receptor antagonist.

12205. The method of item 12083 wherein the agent is a peroxisome proliferator-activated receptor agonist.

12206. The method of item 12083 wherein the agent is an estrogen receptor agent.

12207. The method of item 12083 wherein the agent is a somastostatin analogue.

12208. The method of item 12083 wherein the agent is a neurokinin 1 antagonist.

12209. The method of item 12083 wherein the agent is a neurokinin 3 antagonist.

12210. The method of item 12083 wherein the agent is a VLA-4 antagonist.

12211. The method of item 12083 wherein the agent is an osteoclast inhibitor.

12212. The method of item 12083 wherein the agent is a DNA topoisomerase ATP hydrolyzing inhibitor.

12213. The method of item 12083 wherein the agent is an angiotensin I converting enzyme inhibitor.

12214. The method of item 12083 wherein the agent is an angiotensin II antagonist.

12215. The method of item 12083 wherein the agent is an enkephalinase inhibitor.

12216. The method of item 12083 wherein the agent is a peroxisome proliferator-activated receptor gamma agonist insulin sensitizer.

12217. The method of item 12083 wherein the agent is a protein kinase C inhibitor.

12218. The method of item 12083 wherein the agent is a ROCK (rho-associated kinase) inhibitor.

12219. The method of item 12083 wherein the agent is a CXCR3 inhibitor.

12220. The method of item 12083 wherein the agent is an ltk inhibitor.

12221. The method of item 12083 wherein the agent is a cytosolic phospholipase A₂-alpha inhibitor.

12222. The method of item 12083 wherein the agent is a PPAR agonist.

12223. The method of item 12083 wherein the agent is an immunosuppressant.

12224. The method of item 12083 wherein the agent is an Erb inhibitor.

12225. The method of item 12083 wherein the agent is an apoptosis agonist.

12226. The method of item 12083 wherein the agent is a lipocortin agonist.

12227. The method of item 12083 wherein the agent is a VCAM-1 antagonist.

12228. The method of item 12083 wherein the agent is a collagen antagonist.

12229. The method of item 12083 wherein the agent is an alpha 2 integrin antagonist.

12230. The method of item 12083 wherein the agent is a TNF alpha inhibitor.

12231. The method of item 12083 wherein the agent is a nitric oxide inhibitor.

12232. The method of item 12083 wherein the agent is a cathepsin inhibitor.

12233. The method of item 12083 wherein the agent is not an anti-inflammatory agent.

12234. The method of item 12083 wherein the agent is not a steroid.

12235. The method of item 12083 wherein the agent is not a glucocorticosteroid.

12236. The method of item 12083 wherein the agent is not dexamethasone.

12237. The method of item 12083 wherein the agent is not an anti-infective agent.

12238. The method of item 12083 wherein the agent is not an antibiotic.

12239. The method of item 12083 wherein the agent is not an anti-fungal agent.

12240. The method of item 12083, wherein the composition comprises a polymer.

12241. The method of item 12083, wherein the composition comprises a polymeric carrier.

12242. The method of item 12083 wherein the anti-scarring agent inhibits adhesion between the device and a host into which the device is implanted.

12243. The method of item 12083 wherein the device delivers the anti-scarring agent locally to tissue proximate to the device.

12244. The method of item 12083 wherein the device has a coating that comprises the anti-scarring agent.

12245. The method of item 12083, wherein the device has a coating that comprises the agent and is disposed on a surface of the implant.

12246. The method of item 12083, wherein the device has a coating that comprises the agent and directly contacts the implant.

12247. The method of item 12083, wherein the device has a coating that comprises the agent and indirectly contacts the implant.

12248. The method of item 12083, wherein the device has a coating that comprises the agent and partially covers the implant.

12249. The method of item 12083, wherein the device has a coating that comprises the agent and completely covers the implant.

12250. The method of item 12083, wherein the device has a uniform coating.

12251. The method of item 12083, wherein the device has a non-uniform coating.

12252. The method of item 12083, wherein the device has a discontinuous coating.

12253. The method of item 12083, wherein the device has a patterned coating.

12254. The method of item 12083, wherein the device has a coating with a thickness of 100 μm or less.

12255. The method of item 12083, wherein the device has a coating with a thickness of 10 μm or less.

12256. The method of item 12083, wherein the device has a coating, and the coating adheres to the surface of the implant upon deployment of the implant.

12257. The method of item 12083, wherein the device has a coating, and wherein the coating is stable at room temperature for a period of 1 year.

12258. The method of item 12083, wherein the device has a coating, and wherein the anti-scarring agent is present in the coating in an amount ranging between about 0.0001% to about 1% by weight.

12259. The method of item 12083, wherein the device has a coating, and wherein the anti-scarring agent is present in the coating in an amount ranging between about 1% to about 10% by weight.

12260. The method of item 12083, wherein the device has a coating, and wherein the anti-scarring agent is present in the coating in an amount ranging between about 10% to about 25% by weight.

12261. The method of item 12083, wherein the device has a coating, and wherein the anti-scarring agent is present in the coating in an amount ranging between about 25% to about 70% by weight.

12262. The method of item 12083, wherein the device has a coating, and wherein the coating further comprises a polymer.

12263. The method of item 12083, wherein the device has a first coating having a first composition and a second coating having a second composition.

12264. The method of item 12083, wherein the device has a first coating having a first composition and a second coating having a second composition, wherein the first composition and the second composition are different.

12265. The method of item 12083, wherein the composition comprises a polymer.

12266. The method of item 12083, wherein the composition comprises a polymeric carrier.

12267. The method of item 12083, wherein the composition comprises a polymeric carrier, and wherein the polymeric carrier comprises a copolymer.

12268. The method of item 12083, wherein the composition comprises a polymeric carrier, and wherein the polymeric carrier comprises a block copolymer.

12269. The method of item 12083, wherein the composition comprises a polymeric carrier, and wherein the polymeric carrier comprises a random copolymer.

12270. The method of item 12083, wherein the composition comprises a polymeric carrier, and wherein the polymeric carrier comprises a biodegradable polymer.

12271. The method of item 12083, wherein the composition comprises a polymeric carrier, and wherein the polymeric carrier comprises a non-biodegradable polymer.

12272. The method of item 12083, wherein the composition comprises a polymeric carrier, and wherein the polymeric carrier comprises a hydrophilic polymer.

12273. The method of item 12083, wherein the composition comprises a polymeric carrier, and wherein the polymeric carrier comprises a hydrophobic polymer.

12274. The method of item 12083, wherein the composition comprises a polymeric carrier, and wherein the polymeric carrier comprises a polymer having hydrophilic domains.

12275. The method of item 12083, wherein the composition comprises a polymeric carrier, and wherein the polymeric carrier comprises a polymer having hydrophobic domains.

12276. The method of item 12083, wherein the composition comprises a polymeric carrier, and wherein the polymeric carrier comprises a non-conductive polymer.

12277. The method of item 12083, wherein the composition comprises a polymeric carrier, and wherein the polymeric carrier comprises an elastomer.

12278. The method of item 12083, wherein the composition comprises a polymeric carrier, and wherein the polymeric carrier comprises a hydrogel.

12279. The method of item 12083, wherein the composition comprises a polymeric carrier, and wherein the polymeric carrier comprises a silicone polymer.

12280. The method of item 12083, wherein the composition comprises a polymeric carrier, and wherein the polymeric carrier comprises a hydrocarbon polymer.

12281. The method of item 12083, wherein the composition comprises a polymeric carrier, and wherein the polymeric carrier comprises a styrene-derived polymer.

12282. The method of item 12083, wherein the composition comprises a polymeric carrier, and wherein the polymeric carrier comprises a butadiene polymer.

12283. The method of item 12083, wherein the composition comprises a polymeric carrier, and wherein the polymeric carrier comprises a macromer.

12284. The method of item 12083, wherein the composition comprises a polymeric carrier, and wherein the polymeric carrier comprises a poly(ethylene glycol)polymer.

12285. The method of item 12083 wherein the composition comprises a polymeric carrier, and wherein the polymeric carrier comprises an amorphous polymer.

12286. The method of item 12083, wherein the device comprises a lubricious coating.

12287. The method of item 12083 wherein the anti-scarring agent is located within pores or holes of the device.

12288. The method of item 12083 wherein the anti-scarring agent is located within a channel, lumen, or divet of the device.

12289. The method of item 12083, wherein the device comprises a second pharmaceutically active agent.

12290. The method of item 12083 wherein the device comprises an anti-inflammatory agent.

12291. The method of item 12083 wherein the device comprises an agent that inhibits infection.

12292. The method of item 12083 wherein the device comprises an agent that inhibits infection, and wherein the agent is an anthracycline.

12293. The method of item 12083 wherein the device comprises an agent that inhibits infection, and wherein the agent is doxorubicin.

12294. The method of item 12083 wherein the device comprises an agent that inhibits infection, and wherein the agent is mitoxantrone.

12295. The method of item 12083 wherein the device comprises an agent that inhibits infection, and wherein the agent is a fluoropyrimidine.

12296. The method of item 12083 wherein the device comprises an agent that inhibits infection, and wherein the agent is 5-fluorouracil (5-FU).

12297. The method of item 12083 wherein the device comprises an agent that inhibits infection, and wherein the agent is a folic acid antagonist.

12298. The method of item 12083 wherein the device comprises an agent that inhibits infection, and wherein the agent is methotrexate.

12299. The method of item 12083 wherein the device comprises an agent that inhibits infection, and wherein the agent is a podophylotoxin.

12300. The method of item 12083 wherein the device comprises an agent that inhibits infection, and wherein the agent is etoposide.

12301. The method of item 12083 wherein the device comprises an agent that inhibits infection, and wherein the agent is a camptothecin.

12302. The method of item 12083 wherein the device comprises an agent that inhibits infection, and wherein the agent is a hydroxyurea.

12303. The method of item 12083 wherein the device comprises an agent that inhibits infection, and wherein the agent is a platinum complex.

12304. The method of item 12083 wherein the device comprises an agent that inhibits infection, and wherein the agent is cisplatin.

12305. The method of item 12083, further comprising an anti-thrombotic agent.

12306. The method of item 12083 wherein the device comprises a visualization agent.

12307. The method of item 12083 wherein the device comprises a visualization agent, wherein the visualization agent is a radiopaque material, and wherein the radiopaque material comprises a metal, a halogenated compound, or a barium containing compound.

12308. The method of item 12083 wherein the device comprises a visualization agent, wherein the visualization agent is a radiopaque material, and wherein the radiopaque material comprises barium, tantalum, or technetium.

12309. The method of item 12083 wherein the device comprises a visualization agent, and wherein the visualization agent is a MRI responsive material.

12310. The method of item 12083 wherein the device comprises a visualization agent, and wherein the visualization agent comprises a gadolinium chelate.

12311. The method of item 12083 wherein the device comprises a visualization agent, and wherein the visualization agent comprises iron, magnesium, manganese, copper, or chromium.

12312. The method of item 12083 wherein the device comprises a visualization agent, and wherein the visualization agent comprises an iron oxide compound.

12313. The method of item 12083 wherein the device comprises a visualization agent, and wherein the visualization agent comprises a dye, pigment, or colorant.

12314. The method of item 1.2083 wherein the device comprises an echogenic material.

12315. The method of item 12083 wherein the device comprises an echogenic material, and wherein the echogenic material is in the form of a coating.

12316. The method of item 12083 wherein the device is sterile.

12317. The method of item 12083 wherein the anti-scarring agent is released into tissue in the vicinity of the device after deployment of the device.

12318. The method of item 12083 wherein the anti-scarring agent is released into tissue in the vicinity of the device after deployment of the device, and wherein the tissue is connective tissue.

12319. The method of item 12083 wherein the anti-scarring agent is released into tissue in the vicinity of the device after deployment of the device, and wherein the tissue is muscle tissue.

12320. The method of item 12083 wherein the anti-scarring agent is released into tissue in the vicinity of the device after deployment of the device, and wherein the tissue is nerve tissue.

12321. The method of item 12083 wherein the anti-scarring agent is released into tissue in the vicinity of the device after deployment of the device, and wherein the tissue is epithelium tissue.

12322. The method of item 12083 wherein the anti-scarring agent is released in effective concentrations from the device over a period ranging from the time of deployment of the device to about 1 year.

12323. The method of item 12083 wherein the anti-scarring agent is released in effective concentrations from the device over a period ranging from about 1 month to 6 months.

12324. The method of item 12083 wherein the anti-scarring agent is released in effective concentrations from the device over a period ranging from about 1-90 days.

12325. The method of item 12083 wherein the anti-scarring agent is released in effective concentrations from the device at a constant rate.

12326. The method of item 12083 wherein the anti-scarring agent is released in effective concentrations from the device at an increasing rate.

12327. The method of item 12083 wherein the anti-scarring agent is released in effective concentrations from the device at a decreasing rate.

12328. The method of item 12083 wherein the anti-scarring agent is released in effective concentrations from the composition comprising the anti-scarring agent by diffusion over a period ranging from the time of deployment of the device to about 90 days.

12329. The method of item 12083 wherein the anti-scarring agent is released in effective concentrations from the composition comprising the anti-scarring agent by erosion of the composition over a period ranging from the time of deployment of the device to about 90 days.

12330. The method of item 12083 wherein the device comprises about 0.01 μg to about 10 μg of the anti-scarring agent.

12331. The method of item 12083 wherein the device comprises about 10 μg to about 10 mg of the anti-scarring agent.

12332. The method of item 12083 wherein the device comprises about 10 mg to about 250 mg of the anti-scarring agent.

12333. The method of item 12083 wherein the device comprises about 250 mg to about 1000 mg of the anti-scarring agent.

12334. The method of item 12083 wherein the device comprises about 1000 mg to about 2500 mg of the anti-scarring agent.

12335. The method of item 12083 wherein a surface of the device comprises less than 0.01 μg of the anti-scarring agent per mm² of device surface to which the anti-scarring agent is applied.

12336. The method of item 12083 wherein a surface of the device comprises about 0.01 μg to about 1 μg of the anti-scarring agent per mm² of device surface to which the anti-scarring agent is applied.

12337. The method of item 12083 wherein a surface of the device comprises about 1 μg to about 10 μg of the anti-scarring agent per mm² of device surface to which the anti-scarring agent is applied.

12338. The method of item 12083 wherein a surface of the device comprises about 10 μg to about 250 μg of the anti-scarring agent per mm² of device surface to which the anti-scarring agent is applied.

12339. The method of item 12083 wherein a surface of the device comprises about 250 μg to about 1000 μg of the anti-scarring agent of anti-scarring agent per mm² of device surface to which the anti-scarring agent is applied.

12340. The method of item 12083 wherein a surface of the device comprises about 1000 μg to about 2500 μg of the anti-scarring agent per mm² of device surface to which the anti-scarring agent is applied.

12341. The method of item 12083 wherein the combining is performed by direct affixing the agent or the composition to the implant.

12342. The method of item 12083 wherein the combining is performed by spraying the agent or the component onto the implant.

12343. The method of item 12083 wherein the combining is performed by electrospraying the agent or the composition onto the implant.

12344. The method of item 12083 wherein the combining is performed by dipping the implant into a solution comprising the agent or the composition.

12345. The method of item 12083 wherein the combining is performed by covalently attaching the agent or the composition to the implant.

12346. The method of item 12083 wherein the combining is performed by non-covalently attaching the agent or the composition to the implant.

12347. The method of item 12083 wherein the combining is performed by coating the implant with a substance that contains the agent or the composition.

12348. The method of item 12083 wherein the combining is performed by coating the implant with a substance that absorbs the agent.

12349. The method of item 12083 wherein the combining is performed by interweaving a thread composed of, or coated with, the agent or the composition.

12350. The method of item 12083 wherein the combining is performed by covering all the implant with a sleeve that contains the agent or the composition.

12351. The method of item 12083 wherein the combining is performed by covering a portion of the implant with a sleeve that contains the agent or the composition.

12352. The method of item 12083 wherein the combining is performed by covering all the implant with a cover that contains the agent or the composition.

12353. The method of item 12083 wherein the combining is performed by covering a portion of the implant with a cover that contains the agent or the composition.

12354. The method of item 12083 wherein the combining is performed by covering all the implant with an electrospun fabric that contains the agent or the composition.

12355. The method of item 12083 wherein the combining is performed by covering a portion of the implant with an electrospun fabric that contains the agent or the composition.

12356. The method of item 12083 wherein the combining is performed by covering all the implant with a mesh that contains the agent or the composition.

12357. The method of item 12083 wherein the combining is performed by covering a portion of the implant with a mesh that contains the agent or the composition.

12358. The method of item 12083 wherein the combining is performed by constructing all the implant with the agent or the composition.

12359. The method of item 12083 wherein the combining is performed by constructing a portion of the implant with the agent or the composition.

12360. The method of item 12083 wherein the combining is performed by impregnating the implant with the agent or the composition.

12361. The method of item 12083 wherein the combining is performed by constructing all of the implant from a degradable polymer that releases the agent.

12362. The method of item 12083 wherein the combining is performed by constructing a portion of the implant from a degradable polymer that releases the agent.

12363. The method of item 12083 wherein the combining is performed by dipping the implant into a solution that comprise the agent and an inert solvent for the implant.

12364. The method of item 12083 wherein the combining is performed by dipping the implant into a solution that comprises the agent and a solvent that will swill the implant.

12365. The method of item 12083 wherein the combining is performed by dipping the implant into a solution that comprises the agent and a solvent that will dissolve the implant.

12366. The method of item 12083 wherein the combining is performed by dipping the implant into a solution that comprises the agent, a polymer and an inert solvent for the implant.

12367. The method of item 12083 wherein the combining is performed by dipping the implant into a solution that comprises the agent, a polymer and a solvent that will swill the implant.

12368. The method of item 12083 wherein the combining is performed by dipping the implant into a solution that comprises the agent, a polymer and a solvent that will dissolve the implant.

12369. The method of item 12083 wherein the combining is performed by spraying the implant into a solution that comprises the agent and an inert solvent for the implant.

12370. The method of item 12083 wherein the combining is performed by spraying the implant into a solution that comprises the agent and a solvent that will swill the implant.

12371. The method of item 12083 wherein the combining is performed by spraying the implant into a solution that comprises the agent and a solvent that will dissolve the implant.

12372. The method of item 12083 wherein the combining is performed by spraying the implant into a solution that comprises the agent, a polymer and an inert solvent for the implant.

12373. The method of item 12083 wherein the combining is performed by spraying the implant into a solution that comprises the agent, a polymer and a solvent that will swill the implant.

12374. The method of item 12083 wherein the combining is performed by spraying the implant into a solution that comprises the agent, a polymer and a solvent that will dissolve the implant.

12375. The method of item 12083 wherein the implant is a mechanical prosthesis.

12376. The method of item 12083 wherein the implant is a bioprosthetic heart valve.

12377. The method of item 12083 wherein the implant is a bioprosthetic heart valve formed, at least in part, from porcine valve.

12378. The method of item 12083 wherein the implant is a bioprosthetic heart valve formed, at least in part, from bovine pericardial valve.

12379. A method of making a medical device comprising: combining an inferior vena cava filter implant an anti-scarring agent or a composition comprising an anti-scarring agent, wherein the agent inhibits scarring between the device and a host into which the device is implanted.

12380. The method of item 12379 wherein the agent inhibits cell regeneration.

12381. The method of item 12379 wherein the agent inhibits angiogenesis.

12382. The method of item 12379 wherein the agent inhibits fibroblast migration.

12383. The method of item 12379 wherein the agent inhibits fibroblast proliferation.

12384. The method of item 12379 wherein the agent inhibits deposition of extracellular matrix.

12385. The method of item 12379 wherein the agent inhibits tissue remodeling.

12386. The method of item 12379 wherein the agent is an angiogenesis inhibitor.

12387. The method of item 12379 wherein the agent is a 5-lipoxygenase inhibitor or antagonist.

12388. The method of item 12379 wherein the agent is a chemokine receptor antagonist.

12389. The method of item 12379 wherein the agent is a cell cycle inhibitor.

12390. The method of item 12379 wherein the agent is a taxane.

12391. The method of item 12379 wherein the agent is an anti-microtubule agent.

12392. The method of item 12379 wherein the agent is paclitaxel.

12393. The method of item 12379 wherein the agent is not paclitaxel.

12394. The method of item 12379 wherein the agent is an analogue or derivative of paclitaxel.

12395. The method of item 12379 wherein the agent is a vinca alkaloid.

12396. The method of item 12379 wherein the agent is camptothecin or an analogue or derivative thereof.

12397. The method of item 12379 wherein the agent is a podophyllotoxin.

12398. The method of item 12379 wherein the agent is a podophyllotoxin, wherein the podophyllotoxin is etoposide or an analogue or derivative thereof.

12399. The method of item 12379 wherein the agent is an anthracycline.

12400. The method of item 12379 wherein the agent is an anthracycline, wherein the anthracycline is doxorubicin or an analogue or derivative thereof.

12401. The method of item 12379 wherein the agent is an anthracycline, wherein the anthracycline is mitoxantrone or an analogue or derivative thereof.

12402. The method of item 12379 wherein the agent is a platinum compound.

12403. The method of item 12379 wherein the agent is a nitrosourea.

12404. The method of item 12379 wherein the agent is a nitroimidazole.

12405. The method of item 12379 wherein the agent is a folic acid antagonist.

12406. The method of item 12379 wherein the agent is a cytidine analogue.

12407. The method of item 12379 wherein the agent is a pyrimidine analogue.

12408. The method of item 12379 wherein the agent is a fluoropyrimidine analogue.

12409. The method of item 12379 wherein the agent is a purine analogue.

12410. The method of item 12379 wherein the agent is a nitrogen mustard or an analogue or derivative thereof.

12411. The method of item 12379 wherein the agent is a hydroxyurea.

12412. The method of item 12379 wherein the agent is a mytomiciri or an analogue or derivative thereof.

12413. The method of item 12379 wherein the agent is an alkyl sulfonate.

12414. The method of item 12379 wherein the agent is a benzamide or an analogue or derivative thereof.

12415. The method of item 12379 wherein the agent is a nicotinamide or an analogue or derivative thereof.

12416. The method of item 12379 wherein the agent is a halogenated sugar or an analogue or derivative thereof.

12417. The method of item 12379 wherein the agent is a DNA alkylating agent.

12418. The method of item 12379 wherein the agent is an anti-microtubule agent.

12419. The method of item 12379 wherein the agent is a topoisomerase inhibitor.

12420. The method of item 12379 wherein the agent is a DNA cleaving agent.

12421. The method of item 12379 wherein the agent is an antimetabolite.

12422. The method of item 12379 wherein the agent inhibits adenosine deaminase.

12423. The method of item 12379 wherein the agent inhibits purine ring synthesis.

12424. The method of item 12379 wherein the agent is a nucleotide interconversion inhibitor.

12425. The method of item 12379 wherein the agent inhibits dihydrofolate reduction.

12426. The method of item 12379 wherein the agent blocks thymidine monophosphate.

12427. The method of item 12379 wherein the agent causes DNA damage.

12428. The method of item 12379 wherein the agent is a DNA intercalation agent.

12429. The method of item 12379 wherein the agent is a RNA synthesis inhibitor.

12430. The method of item 12379 wherein the agent is a pyrimidine synthesis inhibitor.

12431. The method of item 12379 wherein the agent inhibits ribonucleotide synthesis or function.

12432. The method of item 12379 wherein the agent inhibits thymidine monophosphate synthesis or function.

12433. The method of item 12379 wherein the agent inhibits DNA synthesis.

12434. The method of item 12379 wherein the agent causes DNA adduct formation.

12435. The method of item 12379 wherein the agent inhibits protein synthesis.

12436. The method of item 12379 wherein the agent inhibits microtubule function.

12437. The method of item 12379 wherein the agent is a cyclin dependent protein kinase inhibitor.

12438. The method of item 12379 wherein the agent is an epidermal growth factor kinase inhibitor.

12439. The method of item 12379 wherein the agent is an elastase inhibitor.

12440. The method of item 12379 wherein the agent is a factor Xa inhibitor.

12441. The method of item 12379 wherein the agent is a farnesyltransferase inhibitor.

12442. The method of item 12379 wherein the agent is a fibrinogen antagonist.

12443. The method of item 12379 wherein the agent is a guanylate cyclase stimulant.

12444. The method of item 12379 wherein the agent is a heat shock protein 90 antagonist.

12445. The method of item 12379 wherein the agent is a heat shock protein 90 antagonist, wherein the heat shock protein 90 antagonist is geldanamycin or an analogue or derivative thereof.

12446. The method of item 12379 wherein the agent is a guanylate cyclase stimulant.

12447. The method of item 12379 wherein the agent is a HMGCoA reductase inhibitor.

12448. The method of item 12379 wherein the agent is a HMGCoA reductase inhibitor, wherein the HMGCoA reductase inhibitor is simvastatin or an analogue or derivative thereof.

12449. The method of item 12379 wherein the agent is a hydroorotate dehydrogenase inhibitor.

12450. The method of item 12379 wherein the agent is an IKK2 inhibitor.

12451. The method of item 12379 wherein the agent is an IL-1 antagonist.

12452. The method of item 12379 wherein the agent is an ICE antagonist.

12453. The method of item 12379 wherein the agent is an IRAK antagonist.

12454. The method of item 12379 wherein the agent is an IL-4 agonist.

12455. The method of item 12379 wherein the agent is an immunomodulatory agent.

12456. The method of item 12379 wherein the agent is sirolimus or an analogue or derivative thereof.

12457. The method of item 12379 wherein the agent is not sirolimus.

12458. The method of item 12379 wherein the agent is everolimus or an analogue or derivative thereof.

12459. The method of item 12379 wherein the agent is tacrolimus or an analogue or derivative thereof.

12460. The method of item 12379 wherein the agent is not tacrolimus.

12461. The method of item 12379 wherein the agent is biolmus or an analogue or derivative thereof.

12462. The method of item 12379 wherein the agent is tresperimus or an analogue or derivative thereof.

12463. The method of item 12379 wherein the agent is auranofin or an analogue or derivative thereof.

12464. The method of item 12379 wherein the agent is 27-0-demethylrapamycin or an analogue or derivative thereof.

12465. The method of item 12379 wherein the agent is gusperimus or an analogue or derivative thereof.

12466. The method of item 12379 wherein the agent is pimecrolimus or an analogue or derivative thereof.

12467. The method of item 12379 wherein the agent is ABT-578 or an analogue or derivative thereof.

12468. The method of item 12379 wherein the agent is an inosine monophosphate dehydrogenase (IMPDH) inhibitor.

12469. The method of item 12379 wherein the agent is an IMPDH inhibitor, wherein the IMPDH inhibitor is mycophenolic acid or an analogue or derivative thereof.

12470. The method of item 12379 wherein the agent is an IMPDH inhibitor, wherein the IMPDH inhibitor is 1-alpha-25 dihydroxy vitamin D₃ or an analogue or derivative thereof.

12471. The method of item 12379 wherein the agent is a leukotriene inhibitor.

12472. The method of item 12379 wherein the agent is a MCP-1 antagonist.

12473. The method of item 12379 wherein the agent is a MMP inhibitor.

12474. The method of item 12379 wherein the agent is an NF kappa B inhibitor.

12475. The method of item 12379 wherein the agent is an NF kappa B inhibitor, wherein the NF kappa B inhibitor is Bay 11-7082.

12476. The method of item 12379 wherein the agent is an NO agonist.

12477. The method of item 12379 wherein the agent is a p38 MAP kinase inhibitor.

12478. The method of item 12379 wherein the agent is a p38 MAP kinase inhibitor, wherein the p38 MAP kinase inhibitor is SB 202190.

12479. The method of item 12379 wherein the agent is a phosphodiesterase inhibitor.

12480. The method of item 12379 wherein the agent is a TGF beta inhibitor.

12481. The method of item 12379 wherein the agent is a thromboxane A2 antagonist.

12482. The method of item 12379 wherein the agent is a TNFa antagonist.

12483. The method of item 12379 wherein the agent is a TACE inhibitor.

12484. The method of item 12379 wherein the agent is a tyrosine kinase inhibitor.

12485. The method of item 12379 wherein the agent is a vitronectin inhibitor.

12486. The method of item 12379 wherein the agent is a fibroblast growth factor inhibitor.

12487. The method of item 12379 wherein the agent is a protein kinase inhibitor.

12488. The method of item 12379 wherein the agent is a PDGF receptor kinase inhibitor.

12489. The method of item 12379 wherein the agent is an endothelial growth factor receptor kinase inhibitor.

12490. The method of item 12379 wherein the agent is a retinoic acid receptor antagonist.

12491. The method of item 12379 wherein the agent is a platelet derived growth factor receptor kinase inhibitor.

12492. The method of item 12379 wherein the agent is a fibronogin antagonist.

12493. The method of item 12379 wherein the agent is an antimycotic agent.

12494. The method of item 12379 wherein the agent is an antimycotic agent, wherein the antimycotic agent is sulconizole.

12495. The method of item 12379 wherein the agent is a bisphosphonate.

12496. The method of item 12379 wherein the agent is a phospholipase A1 inhibitor.

12497. The method of item 12379 wherein the agent is a histamine H1/H2/H3 receptor antagonist.

12498. The method of item 12379 wherein the agent is a macrolide antibiotic.

12499. The method of item 12379 wherein the agent is a GPIIb/IIIa receptor antagonist.

12500. The method of item 12379 wherein the agent is an endothelin receptor antagonist.

12501. The method of item 12379 wherein the agent is a peroxisome proliferator-activated receptor agonist.

12502. The method of item 12379 wherein the agent is an estrogen receptor agent.

12503. The method of item 12379 wherein the agent is a somastostatin analogue.

12504. The method of item 12379 wherein the agent is a neurokinin 1 antagonist.

12505. The method of item 12379 wherein the agent is a neurokinin 3 antagonist.

12506. The method of item 12379 wherein the agent is a VLA-4 antagonist.

12507. The method of item 12379 wherein the agent is an osteociast inhibitor.

12508. The method of item 12379 wherein the agent is a DNA topoisomerase ATP hydrolyzing inhibitor.

12509. The method of item 12379 wherein the agent is an angiotensin I converting enzyme inhibitor.

12510. The method of item 12379 wherein the agent is an angiotensin II antagonist.

12511. The method of item 12379 wherein the agent is an enkephalinase inhibitor.

12512. The method of item 12379 wherein the agent is a peroxisome proliferator-activated receptor gamma agonist insulin sensitizer.

12513. The method of item 12379 wherein the agent is a protein kinase C inhibitor.

12514. The method of item 12379 wherein the agent is a ROCK (rho-associated kinase) inhibitor.

12515. The method of item 12379 wherein the agent is a CXCR3 inhibitor.

12516. The method of item 12379 wherein the agent is an Itk inhibitor.

12517. The method of item 12379 wherein the agent is a cytosolic phospholipase A₂-alpha inhibitor.

12518. The method of item 12379 wherein the agent is a PPAR agonist.

12519. The method of item 12379 wherein the agent is an immunosuppressant.

12520. The method of item 12379 wherein the agent is an Erb inhibitor.

12521. The method of item 12379 wherein the agent is an apoptosis agonist.

12522. The method of item 12379 wherein the agent is a lipocortin agonist.

12523. The method of item 12379 wherein the agent is a VCAM-1 antagonist.

12524. The method of item 12379 wherein the agent is a collagen antagonist.

12525. The method of item 12379 wherein the agent is an alpha 2 integrin antagonist.

12526. The method of item 12379 wherein the agent is a TNF alpha inhibitor.

12527. The method of item 12379 wherein the agent is a nitric oxide inhibitor.

12528. The method of item 12379 wherein the agent is a cathepsin inhibitor.

12529. The method of item 12379 wherein the agent is not an anti-inflammatory agent.

12530. The method of item 12379 wherein the agent is not a steroid.

12531. The method of item 12379 wherein the agent is not a glucocorticosteroid.

12532. The method of item 12379 wherein the agent is not dexamethasone.

12533. The method of item 12379 wherein the agent is not an anti-infective agent.

12534. The method of item 12379 wherein the agent is not an antibiotic.

12535. The method of item 12379 wherein the agent is not an anti-fungal agent.

12536. The method of item 12379, wherein the composition comprises a polymer.

12537. The method of item 12379, wherein the composition comprises a polymeric carrier.

12538. The method of item 12379 wherein the anti-scarring agent inhibits adhesion between the device and a host into which the device is implanted.

12539. The method of item 12379 wherein the device delivers the anti-scarring agent locally to tissue proximate to the device.

12540. The method of item 12379 wherein the device has a coating that comprises the anti-scarring agent.

12541. The method of item 12379, wherein the device has a coating that comprises the agent and is disposed on a surface of the implant.

12542. The method of item 12379, wherein the device has a coating that comprises the agent and directly contacts the implant.

12543. The method of item 12379, wherein the device has a coating that comprises the agent and indirectly contacts the implant.

12544. The method of item 12379, wherein the device has a coating that comprises the agent and partially covers the implant.

12545. The method of item 12379, wherein the device has a coating that comprises the agent and completely covers the implant.

12546. The method of item 12379, wherein the device has a uniform coating.

12547. The method of item 12379, wherein the device has a non-uniform coating.

12548. The method of item 12379, wherein the device has a discontinuous coating.

12549. The method of item 12379, wherein the device has a patterned coating.

12550. The method of item 12379, wherein the device has a coating with a thickness of 100 μm or less.

12551. The method of item 12379, wherein the device has a coating with a thickness of 10 μm or less.

12552. The method of item 12379, wherein the device has a coating, and the coating adheres to the surface of the implant upon deployment of the implant.

12553. The method of item 12379, wherein the device has a coating, and wherein the coating is stable at room temperature for a period of 1 year.

12554. The method of item 12379, wherein the device has a coating, and wherein the anti-scarring agent is present in the coating in an amount ranging between about 0.0001% to about 1% by weight.

12555. The method of item 12379, wherein the device has a coating, and wherein the anti-scarring agent is present in the coating in an amount ranging between about 1% to about 10% by weight.

12556. The method of item 12379, wherein the device has a coating, and wherein the anti-scarring agent is present in the coating in an amount ranging between about 10% to about 25% by weight.

12557. The method of item 12379, wherein the device has a coating, and wherein the anti-scarring agent is present in the coating in an amount ranging between about 25% to about 70% by weight.

12558. The method of item 12379, wherein the device has a coating, and wherein the coating further comprises a polymer.

12559. The method of item 12379, wherein the device has a first coating having a first composition and a second coating having a second composition.

12560. The method of item 12379, wherein the device has a first coating having a first composition and a second coating having a second composition, wherein the first composition and the second composition are different.

12561. The method of item 12379, wherein the composition comprises a polymer.

12562. The method of item 12379, wherein the composition comprises a polymeric carrier.

12563. The method of item 12379, wherein the composition comprises a polymeric carrier, and wherein the polymeric carrier comprises a copolymer.

12564. The method of item 12379, wherein the composition comprises a polymeric carrier, and wherein the polymeric carrier comprises a block copolymer.

12565. The method of item 12379, wherein the composition comprises a polymeric carrier, and wherein the polymeric carrier comprises a random copolymer.

12566. The method of item 12379, wherein the composition comprises a polymeric carrier, and wherein the polymeric carrier comprises a biodegradable polymer.

12567. The method of item 12379, wherein the composition comprises a polymeric carrier, and wherein the polymeric carrier comprises a non-biodegradable polymer.

12568. The method of item 12379, wherein the composition comprises a polymeric carrier, and wherein the polymeric carrier comprises a hydrophilic polymer.

12569. The method of item 12379, wherein the composition comprises a polymeric carrier, and wherein the polymeric carrier comprises a hydrophobic polymer.

12570. The method of item 12379, wherein the composition comprises a polymeric carrier, and wherein the polymeric carrier comprises a polymer having hydrophilic domains.

12571. The method of item 12379, wherein the composition comprises a polymeric carrier, and wherein the polymeric carrier comprises a polymer having hydrophobic domains.

12572. The method of item 12379, wherein the composition comprises a polymeric carrier, and wherein the polymeric carrier comprises a non-conductive polymer.

12573. The method of item 12379, wherein the composition comprises a polymeric carrier, and wherein the polymeric carrier comprises an elastomer.

12574. The method of item 12379, wherein the composition comprises a polymeric carrier, and wherein the polymeric carrier comprises a hydrogel.

12575. The method of item 12379, wherein the composition comprises a polymeric carrier, and wherein the polymeric carrier comprises a silicone polymer.

12576. The method of item 12379, wherein the composition comprises a polymeric carrier, and wherein the polymeric carrier comprises a hydrocarbon polymer.

12577. The method of item 12379, wherein the composition comprises a polymeric carrier, and wherein the polymeric carrier comprises a styrene-derived polymer.

12578. The method of item 12379, wherein the composition comprises a polymeric carrier, and wherein the polymeric carrier comprises a butadiene polymer.

12579. The method of item 12379, wherein the composition comprises a polymeric carrier, and wherein the polymeric carrier comprises a macromer.

12580. The method of item 12379, wherein the composition comprises a polymeric carrier, and wherein the polymeric carrier comprises a poly(ethylene glycol)polymer.

12581. The method of item 12379 wherein the composition comprises a polymeric carrier, and wherein the polymeric carrier comprises an amorphous polymer.

12582. The method of item 12379, wherein the device comprises a lubricious coating.

12583. The method of item 12379 wherein the anti-scarring agent is located within pores or holes of the device.

12584. The method of item 12379 wherein the anti-scarring agent is located within a channel, lumen, or divet of the device.

12585. The method of item 12379, wherein the device comprises a second pharmaceutically active agent.

12586. The method of item 12379 wherein the device comprises an anti-inflammatory agent.

12587. The method of item 12379 wherein the device comprises an agent that inhibits infection.

12588. The method of item 12379 wherein the device comprises an agent that inhibits infection, and wherein the agent is an anthracycline.

12589. The method of item 12379 wherein the device comprises an agent that inhibits infection, and wherein the agent is doxorubicin.

12590. The method of item 12379 wherein the device comprises an agent that inhibits infection, and wherein the agent is mitoxantrone.

12591. The method of item 12379 wherein the device comprises an agent that inhibits infection, and wherein the agent is a fluoropyrimidine.

12592. The method of item 12379 wherein the device comprises an agent that inhibits infection, and wherein the agent is 5-fluorouracil (5-FU).

12593. The method of item 12379 wherein the device comprises an agent that inhibits infection, and wherein the agent is a folic acid antagonist.

12594. The method of item 12379 wherein the device comprises an agent that inhibits infection, and wherein the agent is methotrexate.

12595. The method of item 12379 wherein the device comprises an agent that inhibits infection, and wherein the agent is a podophylotoxin.

12596. The method of item 12379 wherein the device comprises an agent that inhibits infection, and wherein the agent is etoposide.

12597. The method of item 12379 wherein the device comprises an agent that inhibits infection, and wherein the agent is a camptothecin.

12598. The method of item 12379 wherein the device comprises an agent that inhibits infection, and wherein the agent is a hydroxyurea.

12599. The method of item 12379 wherein the device comprises an agent that inhibits infection, and wherein the agent is a platinum complex.

12600. The method of item 12379 wherein the device comprises an agent that inhibits infection, and wherein the agent is cisplatin.

12601. The method of item 12379, further comprising an anti-thrombotic agent.

12602. The method of item 12379 wherein the device comprises a visualization agent.

12603. The method of item 12379 wherein the device comprises a visualization agent, wherein the visualization agent is a radiopaque material, and wherein the radiopaque material comprises a metal, a halogenated compound, or a barium containing compound.

12604. The method of item 12379 wherein the device comprises a visualization agent, wherein the visualization agent is a radiopaque material, and wherein the radiopaque material comprises barium, tantalum, or technetium.

12605. The method of item 12379 wherein the device comprises a visualization agent, and wherein the visualization agent is a MRI responsive material.

12606. The method of item 12379 wherein the device comprises a visualization agent, and wherein the visualization agent comprises a gadolinium chelate.

12607. The method of item 12379 wherein the device comprises a visualization agent, and wherein the visualization agent comprises iron, magnesium, manganese, copper, or chromium.

12608. The method of item 12379 wherein the device comprises a visualization agent, and wherein the visualization agent comprises an iron oxide compound.

12609. The method of item 12379 wherein the device comprises a visualization agent, and wherein the visualization agent comprises a dye, pigment, or colorant.

12610. The method of item 12379 wherein the device comprises an echogenic material.

12611. The method of item 12379 wherein the device comprises an echogenic material, and wherein the echogenic material is in the form of a coating.

12612. The method of item 12379 wherein the device is sterile.

12613. The method of item 12379 wherein the anti-scarring agent is released into tissue in the vicinity of the device after deployment of the device.

12614. The method of item 12379 wherein the anti-scarring agent is released into tissue in the vicinity of the device after deployment of the device, and wherein the tissue is connective tissue.

12615. The method of item 12379 wherein the anti-scarring agent is released into tissue in the vicinity of the device after deployment of the device, and wherein the tissue is muscle tissue.

12616. The method of item 12379 wherein the anti-scarring agent is released into tissue in the vicinity of the device after deployment of the device, and wherein the tissue is nerve tissue.

12617. The method of item 12379 wherein the anti-scarring agent is released into tissue in the vicinity of the device after deployment of the device, and wherein the tissue is epithelium tissue.

12618. The method of item 12379 wherein the anti-scarring agent is released in effective concentrations from the device over a period ranging from the time of deployment of the device to about 1 year.

12619. The method of item 12379 wherein the anti-scarring agent is released in effective concentrations from the device over a period ranging from about 1 month to 6 months.

12620. The method of item 12379 wherein the anti-scarring agent is released in effective concentrations from the device over a period ranging from about 1-90 days.

12621. The method of item 12379 wherein the anti-scarring agent is released in effective concentrations from the device at a constant rate.

12622. The method of item 12379 wherein the anti-scarring agent is released in effective concentrations from the device at an increasing rate.

12623. The method of item 12379 wherein the anti-scarring agent is released in effective concentrations from the device at a decreasing rate.

12624. The method of item 12379 wherein the anti-scarring agent is released in effective concentrations from the composition comprising the anti-scarring agent by diffusion over a period ranging from the time of deployment of the device to about 90 days.

12625. The method of item 12379 wherein the anti-scarring agent is released in effective concentrations from the composition comprising the anti-scarring agent by erosion of the composition over a period ranging from the time of deployment of the device to about 90 days.

12626. The method of item 12379 wherein the device comprises about 0.01 μg to about 10 μg of the anti-scarring agent.

12627. The method of item 12379 wherein the device comprises about 10 μg to about 10 mg of the anti-scarring agent.

12628. The method of item 12379 wherein the device comprises about 10 mg to about 250 mg of the anti-scarring agent.

12629. The method of item 12379 wherein the device comprises about 250 mg to about 1000 mg of the anti-scarring agent.

12630. The method of item 12379 wherein the device comprises about 1000 mg to about 2500 mg of the anti-scarring agent.

12631. The method of item 12379 wherein a surface of the device comprises less than 0.01 μg of the anti-scarring agent per mm² of device surface to which the anti-scarring agent is applied.

12632. The method of item 12379 wherein a surface of the device comprises about 0.01 μg to about 1 μg of the anti-scarring agent per mm² of device surface to which the anti-scarring agent is applied.

12633. The method of item 12379 wherein a surface of the device comprises about 1 μg to about 10 μg of the anti-scarring agent per mm² of device surface to which the anti-scarring agent is applied.

12634. The method of item 12379 wherein a surface of the device comprises about 10 μg to about 250 μg of the anti-scarring agent per mm² of device surface to which the anti-scarring agent is applied.

12635. The method of item 12379 wherein a surface of the device comprises about 250 μg to about 1000 μg of the anti-scarring agent of anti-scarring agent per mm² of device surface to which the anti-scarring agent is applied.

12636. The method of item 12379 wherein a surface of the device comprises about 1000 μg to about 2500 μg of the anti-scarring agent per mm² of device surface to which the anti-scarring agent is applied.

12637. The method of item 12379 wherein the combining is performed by direct affixing the agent or the composition to the implant.

12638. The method of item 12379 wherein the combining is performed by spraying the agent or the component onto the implant.

12639. The method of item 12379 wherein the combining is performed by electrospraying the agent or the composition onto the implant.

12640. The method of item 12379 wherein the combining is performed by dipping the implant into a solution comprising the agent or the composition.

12641. The method of item 12379 wherein the combining is performed by covalently attaching the agent or the composition to the implant.

12642. The method of item 12379 wherein the combining is performed by non-covalently attaching the agent or the composition to the implant.

12643. The method of item 12379 wherein the combining is performed by coating the implant with a substance that contains the agent or the composition.

12644. The method of item 12379 wherein the combining is performed by coating the implant with a substance that absorbs the agent.

12645. The method of item 12379 wherein the combining is performed by interweaving a thread composed of, or coated with, the agent or the composition.

12646. The method of item 12379 wherein the combining is performed by covering all the implant with a sleeve that contains the agent or the composition.

12647. The method of item 12379 wherein the combining is performed by covering a portion of the implant with a sleeve that contains the agent or the composition.

12648. The method of item 12379 wherein the combining is performed by covering all the implant with a cover that contains the agent or the composition.

12649. The method of item 12379 wherein the combining is performed by covering a portion of the implant with a cover that contains the agent or the composition.

12650. The method of item 12379 wherein the combining is performed by covering all the implant with an electrospun fabric that contains the agent or the composition.

12651. The method of item 12379 wherein the combining is performed by covering a portion of the implant with an electrospun fabric that contains the agent or the composition.

12652. The method of item 12379 wherein the combining is performed by covering all the implant with a mesh that contains the agent or the composition.

12653. The method of item 12379 wherein the combining is performed by covering a portion of the implant with a mesh that contains the agent or the composition.

12654. The method of item 12379 wherein the combining is performed by constructing all the implant with the agent or the composition.

12655. The method of item 12379 wherein the combining is performed by constructing a portion of the implant with the agent or the composition.

12656. The method of item 12379 wherein the combining is performed by impregnating the implant with the agent or the composition.

12657. The method of item 12379 wherein the combining is performed by constructing all of the implant from a degradable polymer that releases the agent.

12658. The method of item 12379 wherein the combining is performed by constructing a portion of the implant from a degradable polymer that releases the agent.

12659. The method of item 12379 wherein the combining is performed by dipping the implant into a solution that comprise the agent and an inert solvent for the implant.

12660. The method of item 12379 wherein the combining is performed by dipping the implant into a solution that comprises the agent and a solvent that will swill the implant.

12661. The method of item 12379 wherein the combining is performed by dipping the implant into a solution that comprises the agent and a solvent that will dissolve the implant.

12662. The method of item 12379 wherein the combining is performed by dipping the implant into a solution that comprises the agent, a polymer and an inert solvent for the implant.

12663. The method of item 12379 wherein the combining is performed by dipping the implant into a solution that comprises the agent, a polymer and a solvent that will swill the implant.

12664. The method of item 12379 wherein the combining is performed by dipping the implant into a solution that comprises the agent, a polymer and a solvent that will dissolve the implant.

12665. The method of item 12379 wherein the combining is performed by spraying the implant into a solution that comprises the agent and an inert solvent for the implant.

12666. The method of item 12379 wherein the combining is performed by spraying the implant into a solution that comprises the agent and a solvent that will swill the implant.

12667. The method of item 12379 wherein the combining is performed by spraying the implant into a solution that comprises the agent and a solvent that will dissolve the implant.

12668. The method of item 12379 wherein the combining is performed by spraying the implant into a solution that comprises the agent, a polymer and an inert solvent for the implant.

12669. The method of item 12379 wherein the combining is performed by spraying the implant into a solution that comprises the agent, a polymer and a solvent that will swill the implant.

12670. The method of item 12379 wherein the combining is performed by spraying the implant into a solution that comprises the agent, a polymer and a solvent that will dissolve the implant.

12671. A method of making a medical device comprising: combining a peritoneal dialysis catheter implant and an anti-scarring agent or a composition comprising an anti-scarring agent, wherein the agent inhibits scarring between the device and a host into which the device is implanted.

12672. The method of item 12671 wherein the agent inhibits cell regeneration.

12673. The method of item 12671 wherein the agent inhibits angiogenesis.

12674. The method of item 12671 wherein the agent inhibits fibroblast migration.

12675. The method of item 12671 wherein the agent inhibits fibroblast proliferation.

12676. The method of item 12671 wherein the agent inhibits deposition of extracellular matrix.

12677. The method of item 12671 wherein the agent inhibits tissue remodeling.

12678. The method of item 12671 wherein the agent is an angiogenesis inhibitor.

12679. The method of item 12671 wherein the agent is a 5-lipoxygenase inhibitor or antagonist.

12680. The method of item 12671 wherein the agent is a chemokine receptor antagonist.

12681. The method of item 12671 wherein the agent is a cell cycle inhibitor.

12682. The method of item 12671 wherein the agent is a taxane.

12683. The method of item 12671 wherein the agent is an anti-microtubule agent.

12684. The method of item 12671 wherein the agent is paclitaxel.

12685. The method of item 12671 wherein the agent is not paclitaxel.

12686. The method of item 12671 wherein the agent is an analogue or derivative of paclitaxel.

12687. The method of item 12671 wherein the agent is a vinca alkaloid.

12688. The method of item 12671 wherein the agent is camptothecin or an analogue or derivative thereof.

12689. The method of item 12671 wherein the agent is a podophyllotoxin.

12690. The method of item 12671 wherein the agent is a podophyllotoxin, wherein the podophyllotoxin is etoposide or an analogue or derivative thereof.

12691. The method of item 12671 wherein the agent is an anthracycline.

12692. The method of item 12671 wherein the agent is an anthracycline, wherein the anthracycline is doxorubicin or an analogue or derivative thereof.

12693. The method of item 12671 wherein the agent is an anthracycline, wherein the anthracycline is mitoxantrone or an analogue or derivative thereof.

12694. The method of item 12671 wherein the agent is a platinum compound.

12695. The method of item 12671 wherein the agent is a nitrosourea.

12696. The method of item 12671 wherein the agent is a nitroimidazole.

12697. The method of item 12671 wherein the agent is a folic acid antagonist.

12698. The method of item 12671 wherein the agent is a cytidine analogue.

12699. The method of item 12671 wherein the agent is a pyrimidine analogue.

12700. The method of item 12671 wherein the agent is a fluoropyrimidine analogue.

12701. The method of item 12671 wherein the agent is a purine analogue.

12702. The method of item 12671 wherein the agent is a nitrogen mustard or an analogue or derivative thereof.

12703. The method of item 12671 wherein the agent is a hydroxyurea.

12704. The method of item 12671 wherein the agent is a mytomicin or an analogue or derivative thereof.

12705. The method of item 12671 wherein the agent is an alkyl sulfonate.

12706. The method of item 12671 wherein the agent is a benzamide or an analogue or derivative thereof.

12707. The method of item 12671 wherein the agent is a nicotinamide or an analogue or derivative thereof.

12708. The method of item 12671 wherein the agent is a halogenated sugar or an analogue or derivative thereof.

12709. The method of item 12671 wherein the agent is a DNA alkylating agent.

12710. The method of item 12671 wherein the agent is an anti-microtubule agent.

12711. The method of item 12671 wherein the agent is a topoisomerase inhibitor.

12712. The method of item 12671 wherein the agent is a DNA cleaving agent.

12713. The method of item 12671 wherein the agent is an antimetabolite.

12714. The method of item 12671 wherein the agent inhibits adenosine deaminase.

12715. The method of item 12671 wherein the agent inhibits purine ring synthesis.

12716. The method of item 12671 wherein the agent is a nucleotide interconversion inhibitor.

12717. The method of item 12671 wherein the agent inhibits dihydrofolate reduction.

12718. The method of item 12671 wherein the agent blocks thymidine monophosphate.

12719. The method of item 12671 wherein the agent causes DNA damage.

12720. The method of item 12671 wherein the agent is a DNA intercalation agent.

12721. The method of item 12671 wherein the agent is a RNA synthesis inhibitor.

12722. The method of item 12671 wherein the agent is a pyrimidine synthesis inhibitor.

12723. The method of item 12671 wherein the agent inhibits ribonucleotide synthesis or function.

12724. The method of item 12671 wherein the agent inhibits thymidine monophosphate synthesis or function.

12725. The method of item 12671 wherein the agent inhibits DNA synthesis.

12726. The method of item 12671 wherein the agent causes DNA adduct formation.

12727. The method of item 12671 wherein the agent inhibits protein synthesis.

12728. The method of item 12671 wherein the agent inhibits microtubule function.

12729. The method of item 12671 wherein the agent is a cyclin dependent protein kinase inhibitor.

12730. The method of item 12671 wherein the agent is an epidermal growth factor kinase inhibitor.

12731. The method of item 12671 wherein the agent is an elastase inhibitor.

12732. The method of item 12671 wherein the agent is a factor Xa inhibitor.

12733. The method of item 12671 wherein the agent is a farnesyltransferase inhibitor.

12734. The method of item 12671 wherein the agent is a fibrinogen antagonist.

12735. The method of item 12671 wherein the agent is a guanylate cyclase stimulant.

12736. The method of item 12671 wherein the agent is a heat shock protein 90 antagonist.

12737. The method of item 12671 wherein the agent is a heat shock protein 90 antagonist, wherein the heat shock protein 90 antagonist is geldanamycin or an analogue or derivative thereof.

12738. The method of item 12671 wherein the agent is a guanylate cyclase stimulant.

12739. The method of item 12671 wherein the agent is a HMGCoA reductase inhibitor.

12740. The method of item 12671 wherein the agent is a HMGCoA reductase inhibitor, wherein the HMGCoA reductase inhibitor is simvastatin or an analogue or derivative thereof.

12741. The method of item 12671 wherein the agent is a hydroorotate dehydrogenase inhibitor.

12742. The method of item 12671 wherein the agent is an IKK2 inhibitor.

12743. The method of item 12671 wherein the agent is an IL-1 antagonist.

12744. The method of item 12671 wherein the agent is an ICE antagonist.

12745. The method of item 12671 wherein the agent is an IRAK antagonist.

12746. The method of item 12671 wherein the agent is an IL-4 agonist.

12747. The method of item 12671 wherein the agent is an immunomodulatory agent.

12748. The method of item 12671 wherein the agent is sirolimus or an analogue or derivative thereof.

12749. The method of item 12671 wherein the agent is not sirolimus.

12750. The method of item 12671 wherein the agent is everolimus or an analogue or derivative thereof.

12751. The method of item 12671 wherein the agent is tacrolimus or an analogue or derivative thereof.

12752. The method of item 12671 wherein the agent is not tacrolimus.

12753. The method of item 12671 wherein the agent is biolmus or an analogue or derivative thereof.

12754. The method of item 12671 wherein the agent is tresperimus or an analogue or derivative thereof.

12755. The method of item 12671 wherein the agent is auranofin or an analogue or derivative thereof.

12756. The method of item 12671 wherein the agent is 27-0-demethylrapamycin or an analogue or derivative thereof.

12757. The method of item 12671 wherein the agent is gusperimus or an analogue or derivative thereof.

12758. The method of item 12671 wherein the agent is pimecrolimus or an analogue or derivative thereof.

12759. The method of item 12671 wherein the agent is ABT-578 or an analogue or derivative thereof.

12760. The method of item 12671 wherein the agent is an inosine monophosphate dehydrogenase (IMPDH) inhibitor.

12761. The method of item 12671 wherein the agent is an IMPDH inhibitor, wherein the IMPDH inhibitor is mycophenolic acid or an analogue or derivative thereof.

12762. The method of item 12671 wherein the agent is an IMPDH inhibitor, wherein the IMPDH inhibitor is 1-alpha-25 dihydroxy vitamin D₃ or an analogue or derivative thereof.

12763. The method of item 12671 wherein the agent is a leukotriene inhibitor.

12764. The method of item 12671 wherein the agent is a MCP-1 antagonist.

12765. The method of item 12671 wherein the agent is a MMP inhibitor.

12766. The method of item 12671 wherein the agent is an NF kappa B inhibitor.

12767. The method of item 12671 wherein the agent is an NF kappa B inhibitor, wherein the NF kappa B inhibitor is Bay 11-7082.

12768. The method of item 12671 wherein the agent is an NO agonist.

12769. The method of item 12671 wherein the agent is a p38 MAP kinase inhibitor.

12770. The method of item 12671 wherein the agent is a p38 MAP kinase inhibitor, wherein the p38 MAP kinase inhibitor is SB 202190.

12771. The method of item 12671 wherein the agent is a phosphodiesterase inhibitor.

12772. The method of item 12671 wherein the agent is a TGF beta inhibitor.

12773. The method of item 12671 wherein the agent is a thromboxane A2 antagonist.

12774. The method of item 12671 wherein the agent is a TNFa antagonist.

12775. The method of item 12671 wherein the agent is a TACE inhibitor.

12776. The method of item 12671 wherein the agent is a tyrosine kinase inhibitor.

12777. The method of item 12671 wherein the agent is a vitronectin inhibitor.

12778. The method of item 12671 wherein the agent is a fibroblast growth factor inhibitor.

12779. The method of item 12671 wherein the agent is a protein kinase inhibitor.

12780. The method of item 12671 wherein the agent is a PDGF receptor kinase inhibitor.

12781. The method of item 12671 wherein the agent is an endothelial growth factor receptor kinase inhibitor.

12782. The method of item 12671 wherein the agent is a retinoic acid receptor antagonist.

12783. The method of item 12671 wherein the agent is a platelet derived growth factor receptor kinase inhibitor.

12784. The method of item 12671 wherein the agent is a fibronogin antagonist.

12785. The method of item 12671 wherein the agent is an antimycotic agent.

12786. The method of item 12671 wherein the agent is an antimycotic agent, wherein the antimycotic agent is sulconizole.

12787. The method of item 12671 wherein the agent is a bisphosphonate.

12788. The method of item 12671 wherein the agent is a phospholipase A1 inhibitor.

12789. The method of item 12671 wherein the agent is a histamine H1/H2/H3 receptor antagonist.

12790. The method of item 12671 wherein the agent is a macrolide antibiotic.

12791. The method of item 12671 wherein the agent is a GPIIb/IIIa receptor antagonist.

12792. The method of item 12671 wherein the agent is an endothelin receptor antagonist.

12793. The method of item 12671 wherein the agent is a peroxisome proliferator-activated receptor agonist.

12794. The method of item 12671 wherein the agent is an estrogen receptor agent.

12795. The method of item 12671 wherein the agent is a somastostatin analogue.

12796. The method of item 12671 wherein the agent is a neurokinin 1 antagonist.

12797. The method of item 12671 wherein the agent is a neurokinin 3 antagonist.

12798. The method of item 12671 wherein the agent is a VLA-4 antagonist.

12799. The method of item 12671 wherein the agent is an osteoclast inhibitor.

12800. The method of item 12671 wherein the agent is a DNA topoisomerase ATP hydrolyzing inhibitor.

12801. The method of item 12671 wherein the agent is an angiotensin I converting enzyme inhibitor.

12802. The method of item 12671 wherein the agent is an angiotensin II antagonist.

12803. The method of item 12671 wherein the agent is an enkephalinase inhibitor.

12804. The method of item 12671 wherein the agent is a peroxisome proliferator-activated receptor gamma agonist insulin sensitizer.

12805. The method of item 12671 wherein the agent is a protein kinase C inhibitor.

12806. The method of item 12671 wherein the agent is a ROCK (rho-associated kinase) inhibitor.

12807. The method of item 12671 wherein the agent is a CXCR3 inhibitor.

12808. The method of item 12671 wherein the agent is an Itk inhibitor.

12809. The method of item 12671 wherein the agent is a cytosolic phospholipase A₂-alpha inhibitor.

12810. The method of item 12671 wherein the agent is a PPAR agonist.

12811. The method of item 12671 wherein the agent is an immunosuppressant.

12812. The method of item 12671 wherein the agent is an Erb inhibitor.

12813. The method of item 12671 wherein the agent is an apoptosis agonist.

12814. The method of item 12671 wherein the agent is a lipocortin agonist.

12815. The method of item 12671 wherein the agent is a VCAM-1 antagonist.

12816. The method of item 12671 wherein the agent is a collagen antagonist.

12817. The method of item 12671 wherein the agent is an alpha 2 integrin antagonist.

12818. The method of item 12671 wherein the agent is a TNF alpha inhibitor.

12819. The method of item 12671 wherein the agent is a nitric oxide inhibitor.

12820. The method of item 12671 wherein the agent is a cathepsin inhibitor.

12821. The method of item 12671 wherein the agent is not an anti-inflammatory agent.

12822. The method of item 12671 wherein the agent is not a steroid.

12823. The method of item 12671 wherein the agent is not a glucocorticosteroid.

12824. The method of item 12671 wherein the agent is not dexamethasone.

12825. The method of item 12671 wherein the agent is not an anti-infective agent.

12826. The method of item 12671 wherein the agent is not an antibiotic.

12827. The method of item 12671 wherein the agent is not an anti-fungal agent.

12828. The method of item 12671, wherein the composition comprises a polymer.

12829. The method of item 12671, wherein the composition comprises a polymeric carrier.

12830. The method of item 12671 wherein the anti-scarring agent inhibits adhesion between the device and a host into which the device is implanted.

12831. The method of item 12671 wherein the device delivers the anti-scarring agent locally to tissue proximate to the device.

12832. The method of item 12671 wherein the device has a coating that comprises the anti-scarring agent.

12833. The method of item 12671, wherein the device has a coating that comprises the agent and is disposed on a surface of the implant.

12834. The method of item 12671, wherein the device has a coating that comprises the agent and directly contacts the implant.

12835. The method of item 12671, wherein the device has a coating that comprises the agent and indirectly contacts the implant.

12836. The method of item 12671, wherein the device has a coating that comprises the agent and partially covers the implant.

12837. The method of item 12671, wherein the device has a coating that comprises the agent and completely covers the implant.

12838. The method of item 12671, wherein the device has a uniform coating.

12839. The method of item 12671, wherein the device has a non-uniform coating.

12840. The method of item 12671, wherein the device has a discontinuous coating.

12841. The method of item 12671, wherein the device has a patterned coating.

12842. The method of item 12671, wherein the device has a coating with a thickness of 100 μm or less.

12843. The method of item 12671, wherein the device has a coating with a thickness of 10 μm or less.

12844. The method of item 12671, wherein the device has a coating, and the coating adheres to the surface of the implant upon deployment of the implant.

12845. The method of item 12671, wherein the device has a coating, and wherein the coating is stable at room temperature for a period of 1 year.

12846. The method of item 12671, wherein the device has a coating, and wherein the anti-scarring agent is present in the coating in an amount ranging between about 0.0001% to about 1% by weight.

12847. The method of item 12671, wherein the device has a coating, and wherein the anti-scarring agent is present in the coating in an amount ranging between about 1% to about 10% by weight.

12848. The method of item 12671, wherein the device has a coating, and wherein the anti-scarring agent is present in the coating in an amount ranging between about 10% to about 25% by weight.

12849. The method of item 12671, wherein the device has a coating, and wherein the anti-scarring agent is present in the coating in an amount ranging between about 25% to about 70% by weight.

12850. The method of item 12671, wherein the device has a coating, and wherein the coating further comprises a polymer.

12851. The method of item 12671, wherein the device has a first coating having a first composition and a second coating having a second composition.

12852. The method of item 12671, wherein the device has a first coating having a first composition and a second coating having a second composition, wherein the first composition and the second composition are different.

12853. The method of item 12671, wherein the composition comprises a polymer.

12854. The method of item 12671, wherein the composition comprises a polymeric carrier.

12855. The method of item 12671, wherein the composition comprises a polymeric carrier, and wherein the polymeric carrier comprises a copolymer.

12856. The method of item 12671, wherein the composition comprises a polymeric carrier, and wherein the polymeric carrier comprises a block copolymer.

12857. The method of item 12671, wherein the composition comprises a polymeric carrier, and wherein the polymeric carrier comprises a random copolymer.

12858. The method of item 12671, wherein the composition comprises a polymeric carrier, and wherein the polymeric carrier comprises a biodegradable polymer.

12859. The method of item 12671, wherein the composition comprises a polymeric carrier, and wherein the polymeric carrier comprises a non-biodegradable polymer.

12860. The method of item 12671, wherein the composition comprises a polymeric carrier, and wherein the polymeric carrier comprises a hydrophilic polymer.

12861. The method of item 12671, wherein the composition comprises a polymeric carrier, and wherein the polymeric carrier comprises a hydrophobic polymer.

12862. The method of item 12671, wherein the composition comprises a polymeric carrier, and wherein the polymeric carrier comprises a polymer having hydrophilic domains.

12863. The method of item 12671, wherein the composition comprises a polymeric carrier, and wherein the polymeric carrier comprises a polymer having hydrophobic domains.

12864. The method of item 12671, wherein the composition comprises a polymeric carrier, and wherein the polymeric carrier comprises a non-conductive polymer.

12865. The method of item 12671, wherein the composition comprises a polymeric carrier, and wherein the polymeric carrier comprises an elastomer.

12866. The method of item 12671, wherein the composition comprises a polymeric carrier, and wherein the polymeric carrier comprises a hydrogel.

12867. The method of item 12671, wherein the composition comprises a polymeric carrier, and wherein the polymeric carrier comprises a silicone polymer.

12868. The method of item 12671, wherein the composition comprises a polymeric carrier, and wherein the polymeric carrier comprises a hydrocarbon polymer.

12869. The method of item 12671, wherein the composition comprises a polymeric carrier, and wherein the polymeric carrier comprises a styrene-derived polymer.

12870. The method of item 12671, wherein the composition comprises a polymeric carrier, and wherein the polymeric carrier comprises a butadiene polymer.

12871. The method of item 12671, wherein the composition comprises a polymeric carrier, and wherein the polymeric carrier comprises a macromer.

12872. The method of item 12671, wherein the composition comprises a polymeric carrier, and wherein the polymeric carrier comprises a poly(ethylene glycol)polymer.

12873. The method of item 12671 wherein the composition comprises a polymeric carrier, and wherein the polymeric carrier comprises an amorphous polymer.

12874. The method of item 12671, wherein the device comprises a lubricious coating.

12875. The method of item 12671 wherein the anti-scarring agent is located within pores or holes of the device.

12876. The method of item 12671 wherein the anti-scarring agent is located within a channel, lumen, or divet of the device.

12877. The method of item 12671, wherein the device comprises a second pharmaceutically active agent.

12878. The method of item 12671 wherein the device comprises an anti-inflammatory agent.

12879. The method of item 12671 wherein the device comprises an agent that inhibits infection.

12880. The method of item 12671 wherein the device comprises an agent that inhibits infection, and wherein the agent is an anthracycline.

12881. The method of item 12671 wherein the device comprises an agent that inhibits infection, and wherein the agent is doxorubicin.

12882. The method of item 12671 wherein the device comprises an agent that inhibits infection, and wherein the agent is mitoxantrone.

12883. The method of item 12671 wherein the device comprises an agent that inhibits infection, and wherein the agent is a fluoropyrimidine.

12884. The method of item 12671 wherein the device comprises an agent that inhibits infection, and wherein the agent is 5-fluorouracil (5-FU).

12885. The method of item 12671 wherein the device comprises an agent that inhibits infection, and wherein the agent is a folic acid antagonist.

12886. The method of item 12671 wherein the device comprises an agent that inhibits infection, and wherein the agent is methotrexate.

12887. The method of item 12671 wherein the device comprises an agent that inhibits infection, and wherein the agent is a podophylotoxin.

12888. The method of item 12671 wherein the device comprises an agent that inhibits infection, and wherein the agent is etoposide.

12889. The method of item 12671 wherein the device comprises an agent that inhibits infection, and wherein the agent is a camptothecin.

12890. The method of item 12671 wherein the device comprises an agent that inhibits infection, and wherein the agent is a hydroxyurea.

12891. The method of item 12671 wherein the device comprises an agent that inhibits infection, and wherein the agent is a platinum complex.

12892. The method of item 12671 wherein the device comprises an agent that inhibits infection, and wherein the agent is cisplatin.

12893. The method of item 12671, further comprising an anti-thrombotic agent.

12894. The method of item 12671 wherein the device comprises a visualization agent.

12895. The method of item 12671 wherein the device comprises a visualization agent, wherein the visualization agent is a radiopaque material, and wherein the radiopaque material comprises a metal, a halogenated compound, or a barium containing compound.

12896. The method of item 12671 wherein the device comprises a visualization agent, wherein the visualization agent is a radiopaque material, and wherein the radiopaque material comprises barium, tantalum, or technetium.

12897. The method of item 12671 wherein the device comprises a visualization agent, and wherein the visualization agent is a MRI responsive material.

12898. The method of item 12671 wherein the device comprises a visualization agent, and wherein the visualization agent comprises a gadolinium chelate.

12899. The method of item 12671 wherein the device comprises a visualization agent, and wherein the visualization agent comprises iron, magnesium, manganese, copper, or chromium.

12900. The method of item 12671 wherein the device comprises a visualization agent, and wherein the visualization agent comprises an iron oxide compound.

12901. The method of item 12671 wherein the device comprises a visualization agent, and wherein the visualization agent comprises a dye, pigment, or colorant.

12902. The method of item 12671 wherein the device comprises an echogenic material.

12903. The method of item 12671 wherein the device comprises an echogenic material, and wherein the echogenic material is in the form of a coating.

12904. The method of item 12671 wherein the device is sterile.

12905. The method of item 12671 wherein the anti-scarring agent is released into tissue in the vicinity of the device after deployment of the device.

12906. The method of item 12671 wherein the anti-scarring agent is released into tissue in the vicinity of the device after deployment of the device, and wherein the tissue is connective tissue.

12907. The method of item 12671 wherein the anti-scarring agent is released into tissue in the vicinity of the device after deployment of the device, and wherein the tissue is muscle tissue.

12908. The method of item 12671 wherein the anti-scarring agent is released into tissue in the vicinity of the device after deployment of the device, and wherein the tissue is nerve tissue.

12909. The method of item 12671 wherein the anti-scarring agent is released into tissue in the vicinity of the device after deployment of the device, and wherein the tissue is epithelium tissue.

12910. The method of item 12671 wherein the anti-scarring agent is released in effective concentrations from the device over a period ranging from the time of deployment of the device to about 1 year.

12911. The method of item 12671 wherein the anti-scarring agent is released in effective concentrations from the device over a period ranging from about 1 month to 6 months.

12912. The method of item 12671 wherein the anti-scarring agent is released in effective concentrations from the device over a period ranging from about 1-90 days.

12913. The method of item 12671 wherein the anti-scarring agent is released in effective concentrations from the device at a constant rate.

12914. The method of item 12671 wherein the anti-scarring agent is released in effective concentrations from the device at an increasing rate.

12915. The method of item 12671 wherein the anti-scarring agent is released in effective concentrations from the device at a decreasing rate.

12916. The method of item 12671 wherein the anti-scarring agent is released in effective concentrations from the composition comprising the anti-scarring agent by diffusion over a period ranging from the time of deployment of the device to about 90 days.

12917. The method of item 12671 wherein the anti-scarring agent is released in effective concentrations from the composition comprising the anti-scarring agent by erosion of the composition over a period ranging from the time of deployment of the device to about 90 days.

12918. The method of item 12671 wherein the device comprises about 0.01 μg to about 10 μg of the anti-scarring agent.

12919. The method of item 12671 wherein the device comprises about 10 μg to about 10 mg of the anti-scarring agent.

12920. The method of item 12671 wherein the device comprises about 10 mg to about 250 mg of the anti-scarring agent.

12921. The method of item 12671 wherein the device comprises about 250 mg to about 1000 mg of the anti-scarring agent.

12922. The method of item 12671 wherein the device comprises about 1000 mg to about 2500 mg of the anti-scarring agent.

12923. The method of item 12671 wherein a surface of the device comprises less than 0.01 μg of the anti-scarring agent per mm² of device surface to which the anti-scarring agent is applied.

12924. The method of item 12671 wherein a surface of the device comprises about 0.01 μg to about 1 μg of the anti-scarring agent per mm² of device surface to which the anti-scarring agent is applied.

12925. The method of item 12671 wherein a surface of the device comprises about 1 μg to about 10 μg of the anti-scarring agent per mm² of device surface to which the anti-scarring agent is applied.

12926. The method of item 12671 wherein a surface of the device comprises about 10 μg to about 250 μg of the anti-scarring agent per mm² of device surface to which the anti-scarring agent is applied.

12927. The method of item 12671 wherein a surface of the device comprises about 250 μg to about 1000 μg of the anti-scarring agent of anti-scarring agent per mm² of device surface to which the anti-scarring agent is applied.

12928. The method of item 12671 wherein a surface of the device comprises about 1000 μg to about 2500 μg of the anti-scarring agent per mm² of device surface to which the anti-scarring agent is applied.

12929. The method of item 12671 wherein the combining is performed by direct affixing the agent or the composition to the implant.

12930. The method of item 12671 wherein the combining is performed by spraying the agent or the component onto the implant.

12931. The method of item 12671 wherein the combining is performed by electrospraying the agent or the composition onto the implant.

12932. The method of item 12671 wherein the combining is performed by dipping the implant into a solution comprising the agent or the composition.

12933. The method of item 12671 wherein the combining is performed by covalently attaching the agent or the composition to the implant.

12934. The method of item 12671 wherein the combining is performed by non-covalently attaching the agent or the composition to the implant.

12935. The method of item 12671 wherein the combining is performed by coating the implant with a substance that contains the agent or the composition.

12936. The method of item 12671 wherein the combining is performed by coating the implant with a substance that absorbs the agent.

12937. The method of item 12671 wherein the combining is performed by interweaving a thread composed of, or coated with, the agent or the composition.

12938. The method of item 12671 wherein the combining is performed by covering all the implant with a sleeve that contains the agent or the composition.

12939. The method of item 12671 wherein the combining is performed by covering a portion of the implant with a sleeve that contains the agent or the composition.

12940. The method of item 12671 wherein the combining is performed by covering all the implant with a cover that contains the agent or the composition.

12941. The method of item 12671 wherein the combining is performed by covering a portion of the implant with a cover that contains the agent or the composition.

12942. The method of item 12671 wherein the combining is performed by covering all the implant with an electrospun fabric that contains the agent or the composition.

12943. The method of item 12671 wherein the combining is performed by covering a portion of the implant with an electrospun fabric that contains the agent or the composition.

12944. The method of item 12671 wherein the combining is performed by covering all the implant with a mesh that contains the agent or the composition.

12945. The method of item 12671 wherein the combining is performed by covering a portion of the implant with a mesh that contains the agent or the composition.

12946. The method of item 12671 wherein the combining is performed by constructing all the implant with the agent or the composition.

12947. The method of item 12671 wherein the combining is performed by constructing a portion of the implant with the agent or the composition.

12948. The method of item 12671 wherein the combining is performed by impregnating the implant with the agent or the composition.

12949. The method of item 12671 wherein the combining is performed by constructing all of the implant from a degradable polymer that releases the agent.

12950. The method of item 12671 wherein the combining is performed by constructing a portion of the implant from a degradable polymer that releases the agent.

12951. The method of item 12671 wherein the combining is performed by dipping the implant into a solution that comprise the agent and an inert solvent for the implant.

12952. The method of item 12671 wherein the combining is performed by dipping the implant into a solution that comprises the agent and a solvent that will swill the implant.

12953. The method of item 12671 wherein the combining is performed by dipping the implant into a solution that comprises the agent and a solvent that will dissolve the implant.

12954. The method of item 12671 wherein the combining is performed by dipping the implant into a solution that comprises the agent, a polymer and an inert solvent for the implant.

12955. The method of item 12671 wherein the combining is performed by dipping the implant into a solution that comprises the agent, a polymer and a solvent that will swill the implant.

12956. The method of item 12671 wherein the combining is performed by dipping the implant into a solution that comprises the agent, a polymer and a solvent that will dissolve the implant.

12957. The method of item 12671 wherein the combining is performed by spraying the implant into a solution that comprises the agent and an inert solvent for the implant.

12958. The method of item 12671 wherein the combining is performed by spraying the implant into a solution that comprises the agent and a solvent that will swill the implant.

12959. The method of item 12671 wherein the combining is performed by spraying the implant into a solution that comprises the agent and a solvent that will dissolve the implant.

12960. The method of item 12671 wherein the combining is performed by spraying the implant into a solution that comprises the agent, a polymer and an inert solvent for the implant.

12961. The method of item 12671 wherein the combining is performed by spraying the implant into a solution that comprises the agent, a polymer and a solvent that will swill the implant.

12962. The method of item 12671 wherein the combining is performed by spraying the implant into a solution that comprises the agent, a polymer and a solvent that will dissolve the implant.

12963. A method of making a medical device comprising: combining an implantable nonvascular stent or tube (i.e., an implant) and an anti-scarring agent or a composition comprising an anti-scarring agent, wherein the agent inhibits scarring between the device and a host into which the device is implanted.

12964. The method of item 12963 wherein the agent inhibits cell regeneration.

12965. The method of item 12963 wherein the agent inhibits angiogenesis.

12966. The method of item 12963 wherein the agent inhibits fibroblast migration.

12967. The method of item 12963 wherein the agent inhibits fibroblast proliferation.

12968. The method of item 12963 wherein the agent inhibits deposition of extracellular matrix.

12969. The method of item 12963 wherein the agent inhibits tissue remodeling.

12970. The method of item 12963 wherein the agent is an angiogenesis inhibitor.

12971. The method of item 12963 wherein the agent is a 5-lipoxygenase inhibitor or antagonist.

12972. The method of item 12963 wherein the agent is a chemokine receptor antagonist.

12973. The method of item 12963 wherein the agent is a cell cycle inhibitor.

12974. The method of item 12963 wherein the agent is a taxane.

12975. The method of item 12963 wherein the agent is an anti-microtubule agent.

12976. The method of item 12963 wherein the agent is paclitaxel.

12977. The method of item 12963 wherein the agent is not paclitaxel.

12978. The method of item 12963 wherein the agent is an analogue or derivative of paclitaxel.

12979. The method of item 12963 wherein the agent is a vinca alkaloid.

12980. The method of item 12963 wherein the agent is camptothecin or an analogue or derivative thereof.

12981. The method of item 12963 wherein the agent is a podophyllotoxin.

12982. The method of item 12963 wherein the agent is a podophyllotoxin, wherein the podophyllotoxin is etoposide or an analogue or derivative thereof.

12983. The method of item 12963 wherein the agent is an anthracycline.

12984. The method of item 12963 wherein the agent is an anthracycline, wherein the anthracycline is doxorubicin or an analogue or derivative thereof.

12985. The method of item 12963 wherein the agent is an anthracycline, wherein the anthracycline is mitoxantrone or an analogue or derivative thereof.

12986. The method of item 12963 wherein the agent is a platinum compound.

12987. The method of item 12963 wherein the agent is a nitrosourea.

12988. The method of item 12963 wherein the agent is a nitroimidazole.

12989. The method of item 12963 wherein the agent is a folic acid antagonist.

12990. The method of item 12963 wherein the agent is a cytidine analogue.

12991. The method of item 12963 wherein the agent is a pyrimidine analogue.

12992. The method of item 12963 wherein the agent is a fluoropyrimidine analogue.

12993. The method of item 12963 wherein the agent is a purine analogue.

12994. The method of item 12963 wherein the agent is a nitrogen mustard or an analogue or derivative thereof.

12995. The method of item 12963 wherein the agent is a hydroxyurea.

12996. The method of item 12963 wherein the agent is a mytomicin or an analogue or derivative thereof.

12997. The method of item 12963 wherein the agent is an alkyl sulfonate.

12998. The method of item 12963 wherein the agent is a benzamide or an analogue or derivative thereof.

12999. The method of item 12963 wherein the agent is a nicotinamide or an analogue or derivative thereof.

13000. The method of item 12963 wherein the agent is a halogenated sugar or an analogue or derivative thereof.

13001. The method of item 12963 wherein the agent is a DNA alkylating agent.

13002. The method of item 12963 wherein the agent is an anti-microtubule agent.

13003. The method of item 12963 wherein the agent is a topoisomerase inhibitor.

13004. The method of item 12963 wherein the agent is a DNA cleaving agent.

13005. The method of item 12963 wherein the agent is an antimetabolite.

13006. The method of item 12963 wherein the agent inhibits adenosine deaminase.

13007. The method of item 12963 wherein the agent inhibits purine ring synthesis.

13008. The method of item 12963 wherein the agent is a nucleotide interconversion inhibitor.

13009. The method of item 12963 wherein the agent inhibits dihydrofolate reduction.

13010. The method of item 12963 wherein the agent blocks thymidine monophosphate.

13011. The method of item 12963 wherein the agent causes DNA damage.

13012. The method of item 12963 wherein the agent is a DNA intercalation agent.

13013. The method of item 12963 wherein the agent is a RNA synthesis inhibitor.

13014. The method of item 12963 wherein the agent is a pyrimidine synthesis inhibitor.

13015. The method of item 12963 wherein the agent inhibits ribonucleotide synthesis or function.

13016. The method of item 12963 wherein the agent inhibits thymidine monophosphate synthesis or function.

13017. The method of item 12963 wherein the agent inhibits DNA synthesis.

13018. The method of item 12963 wherein the agent causes DNA adduct formation.

13019. The method of item 12963 wherein the agent inhibits protein synthesis.

13020. The method of item 12963 wherein the agent inhibits microtubule function.

13021. The method of item 12963 wherein the agent is a cyclin dependent protein kinase inhibitor.

13022. The method of item 12963 wherein the agent is an epidermal growth factor kinase inhibitor.

13023. The method of item 12963 wherein the agent is an elastase inhibitor.

13024. The method of item 12963 wherein the agent is a factor Xa inhibitor.

13025. The method of item 12963 wherein the agent is a farnesyltransferase inhibitor.

13026. The method of item 12963 wherein the agent is a fibrinogen antagonist.

13027. The method of item 12963 wherein the agent is a guanylate cyclase stimulant.

13028. The method of item 12963 wherein the agent is a heat shock protein 90 antagonist.

13029. The method of item 12963 wherein the agent is a heat shock protein 90 antagonist, wherein the heat shock protein 90 antagonist is geldanamycin or an analogue or derivative thereof.

13030. The method of item 12963 wherein the agent is a guanylate cyclase stimulant.

13031. The method of item 12963 wherein the agent is a HMGCoA reductase inhibitor.

13032. The method of item 12963 wherein the agent is a HMGCoA reductase inhibitor, wherein the HMGCoA reductase inhibitor is simvastatin or an analogue or derivative thereof.

13033. The method of item 12963 wherein the agent is a hydroorotate dehydrogenase inhibitor.

13034. The method of item 12963 wherein the agent is an IKK2 inhibitor.

13035. The method of item 12963 wherein the agent is an IL-1 antagonist.

13036. The method of item 12963 wherein the agent is an ICE antagonist.

13037. The method of item 12963 wherein the agent is an IRAK antagonist.

13038. The method of item 12963 wherein the agent is an IL-4 agonist.

13039. The method of item 12963 wherein the agent is an immunomodulatory agent.

13040. The method of item 12963 wherein the agent is sirolimus or an analogue or derivative thereof.

13041. The method of item 12963 wherein the agent is not sirolimus.

13042. The method of item 12963 wherein the agent is everolimus or an analogue or derivative thereof.

13043. The method of item 12963 wherein the agent is tacrolimus or an analogue or derivative thereof.

13044. The method of item 12963 wherein the agent is not tacrolimus.

13045. The method of item 12963 wherein the agent is biolmus or an analogue or derivative thereof.

13046. The method of item 12963 wherein the agent is tresperimus or an analogue or derivative thereof.

13047. The method of item 12963 wherein the agent is auranofin or an analogue or derivative thereof.

13048. The method of item 12963 wherein the agent is 27-0-demethylrapamycin or an analogue or derivative thereof.

13049. The method of item 12963 wherein the agent is gusperimus or an analogue or derivative thereof.

13050. The method of item 12963 wherein the agent is pimecrolimus or an analogue or derivative thereof.

13051. The method of item 12963 wherein the agent is ABT-578 or an analogue or derivative thereof.

13052. The method of item 12963 wherein the agent is an inosine monophosphate dehydrogenase (IMPDH) inhibitor.

13053. The method of item 12963 wherein the agent is an IMPDH inhibitor, wherein the IMPDH inhibitor is mycophenolic acid or an analogue or derivative thereof.

13054. The method of item 12963 wherein the agent is an IMPDH inhibitor, wherein the IMPDH inhibitor is 1-alpha-25 dihydroxy vitamin D₃ or an analogue or derivative thereof.

13055. The method of item 12963 wherein the agent is a leukotriene inhibitor.

13056. The method of item 12963 wherein the agent is a MCP-1 antagonist.

13057. The method of item 12963 wherein the agent is a MMP inhibitor.

13058. The method of item 12963 wherein the agent is an NF kappa B inhibitor.

13059. The method of item 12963 wherein the agent is an NF kappa B inhibitor, wherein the NF kappa B inhibitor is Bay 11-7082.

13060. The method of item 12963 wherein the agent is an NO agonist.

13061. The method of item 12963 wherein the agent is a p38 MAP kinase inhibitor.

13062. The method of item 12963 wherein the agent is a p38 MAP kinase inhibitor, wherein the p38 MAP kinase inhibitor is SB 202190.

13063. The method of item 12963 wherein the agent is a phosphodiesterase inhibitor.

13064. The method of item 12963 wherein the agent is a TGF beta inhibitor.

13065. The method of item 12963 wherein the agent is a thromboxane A2 antagonist.

13066. The method of item 12963 wherein the agent is a TNFa antagonist.

13067. The method of item 12963 wherein the agent is a TACE inhibitor.

13068. The method of item 12963 wherein the agent is a tyrosine kinase inhibitor.

13069. The method of item 12963 wherein the agent is a vitronectin inhibitor.

13070. The method of item 12963 wherein the agent is a fibroblast growth factor inhibitor.

13071. The method of item 12963 wherein the agent is a protein kinase inhibitor.

13072. The method of item 12963 wherein the agent is a PDGF receptor kinase inhibitor.

13073. The method of item 12963 wherein the agent is an endothelial growth factor receptor kinase inhibitor.

13074. The method of item 12963 wherein the agent is a retinoic acid receptor antagonist.

13075. The method of item 12963 wherein the agent is a platelet derived growth factor receptor kinase inhibitor.

13076. The method of item 12963 wherein the agent is a fibronogin antagonist.

13077. The method of item 12963 wherein the agent is an antimycotic agent.

13078. The method of item 12963 wherein the agent is an antimycotic agent, wherein the antimycotic agent is sulconizole.

13079. The method of item 12963 wherein the agent is a bisphosphonate.

13080. The method of item 12963 wherein the agent is a phospholipase A1 inhibitor.

13081. The method of item 12963 wherein the agent is a histamine H1/H2/H3 receptor antagonist.

13082. The method of item 12963 wherein the agent is a macrolide antibiotic.

13083. The method of item 12963 wherein the agent is a GPIIb/IIIa receptor antagonist.

13084. The method of item 12963 wherein the agent is an endothelin receptor antagonist.

13085. The method of item 12963 wherein the agent is a peroxisome proliferator-activated receptor agonist.

13086. The method of item 12963 wherein the agent is an estrogen receptor agent.

13087. The method of item 12963 wherein the agent is a somastostatin analogue.

13088. The method of item 12963 wherein the agent is a neurokinin 1 antagonist.

13089. The method of item 12963 wherein the agent is a neurokinin 3 antagonist.

13090. The method of item 12963 wherein the agent is a VLA-4 antagonist.

13091. The method of item 12963 wherein the agent is an osteoclast inhibitor.

13092. The method of item 12963 wherein the agent is a DNA topoisomerase ATP hydrolyzing inhibitor.

13093. The method of item 12963 wherein the agent is an angiotensin I converting enzyme inhibitor.

13094. The method of item 12963 wherein the agent is an angiotensin II antagonist.

13095. The method of item 12963 wherein the agent is an enkephalinase inhibitor.

13096. The method of item 12963 wherein the agent is a peroxisome proliferator-activated receptor gamma agonist insulin sensitizer.

13097. The method of item 12963 wherein the agent is a protein kinase C inhibitor.

13098. The method of item 12963 wherein the agent is a ROCK (rho-associated kinase) inhibitor.

13099. The method of item 12963 wherein the agent is a CXCR3 inhibitor.

13100. The method of item 12963 wherein the agent is an Itk inhibitor.

13101. The method of item 12963 wherein the agent is a cytosolic phospholipase A₂-alpha inhibitor.

13102. The method of item 12963 wherein the agent is a PPAR agonist.

13103. The method of item 12963 wherein the agent is an immunosuppressant.

13104. The method of item 12963 wherein the agent is an Erb inhibitor.

13105. The method of item 12963 wherein the agent is an apoptosis agonist.

13106. The method of item 12963 wherein the agent is a lipocortin agonist.

13107. The method of item 12963 wherein the agent is a VCAM-1 antagonist.

13108. The method of item 12963 wherein the agent is a collagen antagonist.

13109. The method of item 12963 wherein the agent is an alpha 2 integrin antagonist.

13110. The method of item 12963 wherein the agent is a TNF alpha inhibitor.

13111. The method of item 12963 wherein the agent is a nitric oxide inhibitor.

13112. The method of item 12963 wherein the agent is a cathepsin inhibitor.

13113. The method of item 12963 wherein the agent is not an anti-inflammatory agent.

13114. The method of item 12963 wherein the agent is not a steroid.

13115. The method of item 12963 wherein the agent is not a glucocorticosteroid.

13116. The method of item 12963 wherein the agent is not dexamethasone.

13117. The method of item 12963 wherein the agent is not an anti-infective agent.

13118. The method of item 12963 wherein the agent is not an antibiotic.

13119. The method of item 12963 wherein the agent is not an anti-fungal agent.

13120. The method of item 12963, wherein the composition comprises a polymer.

13121. The method of item 12963, wherein the composition comprises a polymeric carrier.

13122. The method of item 12963 wherein the anti-scarring agent inhibits adhesion between the device and a host into which the device is implanted.

13123. The method of item 12963 wherein the device delivers the anti-scarring agent locally to tissue proximate to the device.

13124. The method of item 12963 wherein the device has a coating that comprises the anti-scarring agent.

13125. The method of item 12963, wherein the device has a coating that comprises the agent and is disposed on a surface of the implant.

13126. The method of item 12963, wherein the device has a coating that comprises the agent and directly contacts the implant.

13127. The method of item 12963, wherein the device has a coating that comprises the agent and indirectly contacts the implant.

13128. The method of item 12963, wherein the device has a coating that comprises the agent and partially covers the implant.

13129. The method of item 12963, wherein the device has a coating that comprises the agent and completely covers the implant.

13130. The method of item 12963, wherein the device has a uniform coating.

13131. The method of item 12963, wherein the device has a non-uniform coating.

13132. The method of item 12963, wherein the device has a discontinuous coating.

13133. The method of item 12963, wherein the device has a patterned coating.

13134. The method of item 12963, wherein the device has a coating with a thickness of 100 μm or less.

13135. The method of item 12963, wherein the device has a coating with a thickness of 10 μm or less.

13136. The method of item 12963, wherein the device has a coating, and the coating adheres to the surface of the implant upon deployment of the implant.

13137. The method of item 12963, wherein the device has a coating, and wherein the coating is stable at room temperature for a period of 1 year.

13138. The method of item 12963, wherein the device has a coating, and wherein the anti-scarring agent is present in the coating in an amount ranging between about 0.0001% to about 1% by weight.

13139. The method of item 12963, wherein the device has a coating, and wherein the anti-scarring agent is present in the coating in an amount ranging between about 1% to about 10% by weight.

13140. The method of item 12963, wherein the device has a coating, and wherein the anti-scarring agent is present in the coating in an amount ranging between about 10% to about 25% by weight.

13141. The method of item 12963, wherein the device has a coating, and wherein the anti-scarring agent is present in the coating in an amount ranging between about 25% to about 70% by weight.

13142. The method of item 12963, wherein the device has a coating, and wherein the coating further comprises a polymer.

13143. The method of item 12963, wherein the device has a first coating having a first composition and a second coating having a second composition.

13144. The method of item 12963, wherein the device has a first coating having a first composition and a second coating having a second composition, wherein the first composition and the second composition are different.

13145. The method of item 12963, wherein the composition comprises a polymer.

13146. The method of item 12963, wherein the composition comprises a polymeric carrier.

13147. The method of item 12963, wherein the composition comprises a polymeric carrier, and wherein the polymeric carrier comprises a copolymer.

13148. The method of item 12963, wherein the composition comprises a polymeric carrier, and wherein the polymeric carrier comprises a block copolymer.

13149. The method of item 12963, wherein the composition comprises a polymeric carrier, and wherein the polymeric carrier comprises a random copolymer.

13150. The method of item 12963, wherein the composition comprises a polymeric carrier, and wherein the polymeric carrier comprises a biodegradable polymer.

13151. The method of item 12963, wherein the composition comprises a polymeric carrier, and wherein the polymeric carrier comprises a non-biodegradable polymer.

13152. The method of item 12963, wherein the composition comprises a polymeric carrier, and wherein the polymeric carrier comprises a hydrophilic polymer.

13153. The method of item 12963, wherein the composition comprises a polymeric carrier, and wherein the polymeric carrier comprises a hydrophobic polymer.

13154. The method of item 12963, wherein the composition comprises a polymeric carrier, and wherein the polymeric carrier comprises a polymer having hydrophilic domains.

13155. The method of item 12963, wherein the composition comprises a polymeric carrier, and wherein the polymeric carrier comprises a polymer having hydrophobic domains.

13156. The method of item 12963, wherein the composition comprises a polymeric carrier, and wherein the polymeric carrier comprises a non-conductive polymer.

13157. The method of item 12963, wherein the composition comprises a polymeric carrier, and wherein the polymeric carrier comprises an elastomer.

13158. The method of item 12963, wherein the composition comprises a polymeric carrier, and wherein the polymeric carrier comprises a hydrogel.

13159. The method of item 12963, wherein the composition comprises a polymeric carrier, and wherein the polymeric carrier comprises a silicone polymer.

13160. The method of item 12963, wherein the composition comprises a polymeric carrier, and wherein the polymeric carrier comprises a hydrocarbon polymer.

13161. The method of item 12963, wherein the composition comprises a polymeric carrier, and wherein the polymeric carrier comprises a styrene-derived polymer.

13162. The method of item 12963, wherein the composition comprises a polymeric carrier, and wherein the polymeric carrier comprises a butadiene polymer.

13163. The method of item 12963, wherein the composition comprises a polymeric carrier, and wherein the polymeric carrier comprises a macromer.

13164. The method of item 12963, wherein the composition comprises a polymeric carrier, and wherein the polymeric carrier comprises a poly(ethylene glycol)polymer.

13165. The method of item 12963 wherein the composition comprises a polymeric carrier, and wherein the polymeric carrier comprises an amorphous polymer.

13166. The method of item 12963, wherein the device comprises a lubricious coating.

13167. The method of item 12963 wherein the anti-scarring agent is located within pores or holes of the device.

13168. The method of item 12963 wherein the anti-scarring agent is located within a channel, lumen, or divet of the device.

13169. The method of item 12963, wherein the device comprises a second pharmaceutically active agent.

13170. The method of item 12963 wherein the device comprises an anti-inflammatory agent.

13171. The method of item 12963 wherein the device comprises an agent that inhibits infection.

13172. The method of item 12963 wherein the device comprises an agent that inhibits infection, and wherein the agent is an anthracycline.

13173. The method of item 12963 wherein the device comprises an agent that inhibits infection, and wherein the agent is doxorubicin.

13174. The method of item 12963 wherein the device comprises an agent that inhibits infection, and wherein the agent is mitoxantrone.

13175. The method of item 12963 wherein the device comprises an agent that inhibits infection, and wherein the agent is a fluoropyrimidine.

13176. The method of item 12963 wherein the device comprises an agent that inhibits infection, and wherein the agent is 5-fluorouracil (5-FU).

13177. The method of item 12963 wherein the device comprises an agent that inhibits infection, and wherein the agent is a folic acid antagonist.

13178. The method of item 12963 wherein the device comprises an agent that inhibits infection, and wherein the agent is methotrexate.

13179. The method of item 12963 wherein the device comprises an agent that inhibits infection, and wherein the agent is a podophylotoxin.

13180. The method of item 12963 wherein the device comprises an agent that inhibits infection, and wherein the agent is etoposide.

13181. The method of item 12963 wherein the device comprises an agent that inhibits infection, and wherein the agent is a camptothecin.

13182. The method of item 12963 wherein the device comprises an agent that inhibits infection, and wherein the agent is a hydroxyurea.

13183. The method of item 12963 wherein the device comprises an agent that inhibits infection, and wherein the agent is a platinum complex.

13184. The method of item 12963 wherein the device comprises an agent that inhibits infection, and wherein the agent is cisplatin.

13185. The method of item 12963, further comprising an anti-thrombotic agent.

13186. The method of item 12963 wherein the device comprises a visualization agent.

13187. The method of item 12963 wherein the device comprises a visualization agent, wherein the visualization agent is a radiopaque material, and wherein the radiopaque material comprises a metal, a halogenated compound, or a barium containing compound.

13188. The method of item 12963 wherein the device comprises a visualization agent, wherein the visualization agent is a radiopaque material, and wherein the radiopaque material comprises barium, tantalum, or technetium.

13189. The method of item 12963 wherein the device comprises a visualization agent, and wherein the visualization agent is a MRI responsive material.

13190. The method of item 12963 wherein the device comprises a visualization agent, and wherein the visualization agent comprises a gadolinium chelate.

13191. The method of item 12963 wherein the device comprises a visualization agent, and wherein the visualization agent comprises iron, magnesium, manganese, copper, or chromium.

13192. The method of item 12963 wherein the device comprises a visualization agent, and wherein the visualization agent comprises an iron oxide compound.

13193. The method of item 12963 wherein the device comprises a visualization agent, and wherein the visualization agent comprises a dye, pigment, or colorant.

13194. The method of item 12963 wherein the device comprises an echogenic material.

13195. The method of item 12963 wherein the device comprises an echogenic material, and wherein the echogenic material is in the form of a coating.

13196. The method of item 12963 wherein the device is sterile.

13197. The method of item 12963 wherein the anti-scarring agent is released into tissue in the vicinity of the device after deployment of the device.

13198. The method of item 12963 wherein the anti-scarring agent is released into tissue in the vicinity of the device after deployment of the device, and wherein the tissue is connective tissue.

13199. The method of item 12963 wherein the anti-scarring agent is released into tissue in the vicinity of the device after deployment of the device, and wherein the tissue is muscle tissue.

13200. The method of item 12963 wherein the anti-scarring agent is released into tissue in the vicinity of the device after deployment of the device, and wherein the tissue is nerve tissue.

13201. The method of item 12963 wherein the anti-scarring agent is released into tissue in the vicinity of the device after deployment of the device, and wherein the tissue is epithelium tissue.

13202. The method of item 12963 wherein the anti-scarring agent is released in effective concentrations from the device over a period ranging from the time of deployment of the device to about 1 year.

13203. The method of item 12963 wherein the anti-scarring agent is released in effective concentrations from the device over a period ranging from about 1 month to 6 months.

13204. The method of item 12963 wherein the anti-scarring agent is released in effective concentrations from the device over a period ranging from about 1-90 days.

13205. The method of item 12963 wherein the anti-scarring agent is released in effective concentrations from the device at a constant rate.

13206. The method of item 12963 wherein the anti-scarring agent is released in effective concentrations from the device at an increasing rate.

13207. The method of item 12963 wherein the anti-scarring agent is released in effective concentrations from the device at a decreasing rate.

13208. The method of item 12963 wherein the anti-scarring agent is released in effective concentrations from the composition comprising the anti-scarring agent by diffusion over a period ranging from the time of deployment of the device to about 90 days.

13209. The method of item 12963 wherein the anti-scarring agent is released in effective concentrations from the composition comprising the anti-scarring agent by erosion of the composition over a period ranging from the time of deployment of the device to about 90 days.

13210. The method of item 12963 wherein the device comprises about 0.01 μg to about 10 μg of the anti-scarring agent.

13211. The method of item 12963 wherein the device comprises about 10 μg to about 10 mg of the anti-scarring agent.

13212. The method of item 12963 wherein the device comprises about 10 mg to about 250 mg of the anti-scarring agent.

13213. The method of item 12963 wherein the device comprises about 250 mg to about 1000 mg of the anti-scarring agent.

13214. The method of item 12963 wherein the device comprises about 1000 mg to about 2500 mg of the anti-scarring agent.

13215. The method of item 12963 wherein a surface of the device comprises less than 0.01 μg of the anti-scarring agent per mm² of device surface to which the anti-scarring agent is applied.

13216. The method of item 12963 wherein a surface of the device comprises about 0.01 μg to about 1 μg of the anti-scarring agent per mm² of device surface to which the anti-scarring agent is applied.

13217. The method of item 12963 wherein a surface of the device comprises about 1 μg to about 10 μg of the anti-scarring agent per mm² of device surface to which the anti-scarring agent is applied.

13218. The method of item 12963 wherein a surface of the device comprises about 10 μg to about 250 μg of the anti-scarring agent per mm² of device surface to which the anti-scarring agent is applied.

13219. The method of item 12963 wherein a surface of the device comprises about 250 μg to about 1000 μg of the anti-scarring agent of anti-scarring agent per mm² of device surface to which the anti-scarring agent is applied.

13220. The method of item 12963 wherein a surface of the device comprises about 1000 μg to about 2500 μg of the anti-scarring agent per mm² of device surface to which the anti-scarring agent is applied.

13221. The method of item 12963 wherein the combining is performed by direct affixing the agent or the composition to the implant.

13222. The method of item 12963 wherein the combining is performed by spraying the agent or the component onto the implant.

13223. The method of item 12963 wherein the combining is performed by electrospraying the agent or the composition onto the implant.

13224. The method of item 12963 wherein the combining is performed by dipping the implant into a solution comprising the agent or the composition.

13225. The method of item 12963 wherein the combining is performed by covalently attaching the agent or the composition to the implant.

13226. The method of item 12963 wherein the combining is performed by non-covalently attaching the agent or the composition to the implant.

13227. The method of item 12963 wherein the combining is performed by coating the implant with a substance that contains the agent or the composition.

13228. The method of item 12963 wherein the combining is performed by coating the implant with a substance that absorbs the agent.

13229. The method of item 12963 wherein the combining is performed by interweaving a thread composed of, or coated with, the agent or the composition.

13230. The method of item 12963 wherein the combining is performed by covering all the implant with a sleeve that contains the agent or the composition.

13231. The method of item 12963 wherein the combining is performed by covering a portion of the implant with a sleeve that contains the agent or the composition.

13232. The method of item 12963 wherein the combining is performed by covering all the implant with a cover that contains the agent or the composition.

13233. The method of item 12963 wherein the combining is performed by covering a portion of the implant with a cover that contains the agent or the composition.

13234. The method of item 12963 wherein the combining is performed by covering all the implant with an electrospun fabric that contains the agent or the composition.

13235. The method of item 12963 wherein the combining is performed by covering a portion of the implant with an electrospun fabric that contains the agent or the composition.

13236. The method of item 12963 wherein the combining is performed by covering all the implant with a mesh that contains the agent or the composition.

13237. The method of item 12963 wherein the combining is performed by covering a portion of the implant with a mesh that contains the agent or the composition.

13238. The method of item 12963 wherein the combining is performed by constructing all the implant with the agent or the composition.

13239. The method of item 12963 wherein the combining is performed by constructing a portion of the implant with the agent or the composition.

13240. The method of item 12963 wherein the combining is performed by impregnating the implant with the agent or the composition.

13241. The method of item 12963 wherein the combining is performed by constructing all of the implant from a degradable polymer that releases the agent.

13242. The method of item 12963 wherein the combining is performed by constructing a portion of the implant from a degradable polymer that releases the agent.

13243. The method of item 12963 wherein the combining is performed by dipping the implant into a solution that comprise the agent and an inert solvent for the implant.

13244. The method of item 12963 wherein the combining is performed by dipping the implant into a solution that comprises the agent and a solvent that will swill the implant.

13245. The method of item 12963 wherein the combining is performed by dipping the implant into a solution that comprises the agent and a solvent that will dissolve the implant.

13246. The method of item 12963 wherein the combining is performed by dipping the implant into a solution that comprises the agent, a polymer and an inert solvent for the implant.

13247. The method of item 12963 wherein the combining is performed by dipping the implant into a solution that comprises the agent, a polymer and a solvent that will swill the implant.

13248. The method of item 12963 wherein the combining is performed by dipping the implant into a solution that comprises the agent, a polymer and a solvent that will dissolve the implant.

13249. The method of item 12963 wherein the combining is performed by spraying the implant into a solution that comprises the agent and an inert solvent for the implant.

13250. The method of item 12963 wherein the combining is performed by spraying the implant into a solution that comprises the agent and a solvent that will swill the implant.

13251. The method of item 12963 wherein the combining is performed by spraying the implant into a solution that comprises the agent and a solvent that will dissolve the implant.

13252. The method of item 12963 wherein the combining is performed by spraying the implant into a solution that comprises the agent, a polymer and an inert solvent for the implant.

13253. The method of item 12963 wherein the combining is performed by spraying the implant into a solution that comprises the agent, a polymer and a solvent that will swill the implant.

13254. The method of item 12963 wherein the combining is performed by spraying the implant into a solution that comprises the agent, a polymer and a solvent that will dissolve the implant.

13255. The method of item 12963 wherein the implant is a gastrointestinal stent.

13256. The method of item 12963 wherein the implant is an esophageal stent.

13257. The method of item 12963 wherein the implant is a biliary stent.

13258. The method of item 12963 wherein the implant is a colonic stent.

13259. The method of item 12963 wherein the implant is a pancreatic stent.

13260. The method of item 12963 wherein the implant is a tracheal stent.

13261. The method of item 12963 wherein the implant is a bronchial stent.

13262. The method of item 12963 wherein the implant is a genital-urinary stent.

13263. The method of item 12963 wherein the implant is an ureteric stent.

13264. The method of item 12963 wherein the implant is a fallopian stent.

13265. The method of item 12963 wherein the implant is a prostate stent.

13266. The method of item 12963 wherein the implant is an ear stent.

13267. The method of item 12963 wherein the implant is a nose stent.

13268. The method of item 12963 wherein the implant is an ear ventilation tube.

13269. The method of item 12963 wherein the implant is an Eustachian tube.

13270. The method of item 12963 wherein the implant is a tympanostomy tube.

13271. A method of making a medical device comprising: combining a central nervous system shunt (i.e., an implant) and an anti-scarring agent or a composition comprising an anti-scarring agent, wherein the agent inhibits scarring between the device and a host into which the device is implanted.

13272. The method of item 13271 wherein the agent inhibits cell regeneration.

13273. The method of item 13271 wherein the agent inhibits angiogenesis.

13274. The method of item 13271 wherein the agent inhibits fibroblast migration.

13275. The method of item 13271 wherein the agent inhibits fibroblast proliferation.

13276. The method of item 13271 wherein the agent inhibits deposition of extracellular matrix.

13277. The method of item 13271 wherein the agent inhibits tissue remodeling.

13278. The method of item 13271 wherein the agent is an angiogenesis inhibitor.

13279. The method of item 13271 wherein the agent is a 5-lipoxygenase inhibitor or antagonist.

13280. The method of item 13271 wherein the agent is a chemokine receptor antagonist.

13281. The method of item 13271 wherein the agent is a cell cycle inhibitor.

13282. The method of item 13271 wherein the agent is a taxane.

13283. The method of item 13271 wherein the agent is an anti-microtubule agent.

13284. The method of item 13271 wherein the agent is paclitaxel.

13285. The method of item 13271 wherein the agent is not paclitaxel.

13286. The method of item 13271 wherein the agent is an analogue or derivative of paclitaxel.

13287. The method of item 13271 wherein the agent is a vinca alkaloid.

13288. The method of item 13271 wherein the agent is camptothecin or an analogue or derivative thereof.

13289. The method of item 13271 wherein the agent is a podophyllotoxin.

13290. The method of item 13271 wherein the agent is a podophyllotoxin, wherein the podophyllotoxin is etoposide or an analogue or derivative thereof.

13291. The method of item 13271 wherein the agent is an anthracycline.

13292. The method of item 13271 wherein the agent is an anthracycline, wherein the anthracycline is doxorubicin or an analogue or derivative thereof.

13293. The method of item 13271 wherein the agent is an anthracycline, wherein the anthracycline is mitoxantrone or an analogue or derivative thereof.

13294. The method of item 13271 wherein the agent is a platinum compound.

13295. The method of item 13271 wherein the agent is a nitrosourea.

13296. The method of item 13271 wherein the agent is a nitroimidazole.

13297. The method of item 13271 wherein the agent is a folic acid antagonist.

13298. The method of item 13271 wherein the agent is a cytidine analogue.

13299. The method of item 13271 wherein the agent is a pyrimidine analogue.

13300. The method of item 13271 wherein the agent is a fluoropyrimidine analogue.

13301. The method of item 13271 wherein the agent is a purine analogue.

13302. The method of item 13271 wherein the agent is a nitrogen mustard or an analogue or derivative thereof.

13303. The method of item 13271 wherein the agent is a hydroxyurea.

13304. The method of item 13271 wherein the agent is a mytomicin or an analogue or derivative thereof.

13305. The method of item 13271 wherein the agent is an alkyl sulfonate.

13306. The method of item 13271 wherein the agent is a benzamide or an analogue or derivative thereof.

13307. The method of item 13271 wherein the agent is a nicotinamide or an analogue or derivative thereof.

13308. The method of item 13271 wherein the agent is a halogenated sugar or an analogue or derivative thereof.

13309. The method of item 13271 wherein the agent is a DNA alkylating agent.

13310. The method of item 13271 wherein the agent is an anti-microtubule agent.

13311. The method of item 13271 wherein the agent is a topoisomerase inhibitor.

13312. The method of item 13271 wherein the agent is a DNA cleaving agent.

13313. The method of item 13271 wherein the agent is an antimetabolite.

13314. The method of item 13271 wherein the agent inhibits adenosine deaminase.

13315. The method of item 13271 wherein the agent inhibits purine ring synthesis.

13316. The method of item 13271 wherein the agent is a nucleotide interconversion inhibitor.

13317. The method of item 13271 wherein the agent inhibits dihydrofolate reduction.

13318. The method of item 13271 wherein the agent blocks thymidine monophosphate.

13319. The method of item 13271 wherein the agent causes DNA damage.

13320. The method of item 13271 wherein the agent is a DNA intercalation agent.

13321. The method of item 13271 wherein the agent is a RNA synthesis inhibitor.

13322. The method of item 13271 wherein the agent is a pyrimidine synthesis inhibitor.

13323. The method of item 13271 wherein the agent inhibits ribonucleotide synthesis or function.

13324. The method of item 13271 wherein the agent inhibits thymidine monophosphate synthesis or function.

13325. The method of item 13271 wherein the agent inhibits DNA synthesis.

13326. The method of item 13271 wherein the agent causes DNA adduct formation.

13327. The method of item 13271 wherein the agent inhibits protein synthesis.

13328. The method of item 13271 wherein the agent inhibits microtubule function.

13329. The method of item 13271 wherein the agent is a cyclin dependent protein kinase inhibitor.

13330. The method of item 13271 wherein the agent is an epidermal growth factor kinase inhibitor.

13331. The method of item 13271 wherein the agent is an elastase inhibitor.

13332. The method of item 13271 wherein the agent is a factor Xa inhibitor.

13333. The method of item 13271 wherein the agent is a farnesyltransferase inhibitor.

13334. The method of item 13271 wherein the agent is a fibrinogen antagonist.

13335. The method of item 13271 wherein the agent is a guanylate cyclase stimulant.

13336. The method of item 13271 wherein the agent is a heat shock protein 90 antagonist.

13337. The method of item 13271 wherein the agent is a heat shock protein 90 antagonist, wherein the heat shock protein 90 antagonist is geldanamycin or an analogue or derivative thereof.

13338. The method of item 13271 wherein the agent is a guanylate cyclase stimulant.

13339. The method of item 13271 wherein the agent is a HMGCoA reductase inhibitor.

13340. The method of item 13271 wherein the agent is a HMGCoA reductase inhibitor, wherein the HMGCoA reductase inhibitor is simvastatin or an analogue or derivative thereof.

13341. The method of item 13271 wherein the agent is a hydroorotate dehydrogenase inhibitor.

13342. The method of item 13271 wherein the agent is an IKK2 inhibitor.

13343. The method of item 13271 wherein the agent is an IL-1 antagonist.

13344. The method of item 13271 wherein the agent is an ICE antagonist.

13345. The method of item 13271 wherein the agent is an IRAK antagonist.

13346. The method of item 13271 wherein the agent is an IL-4 agonist.

13347. The method of item 13271 wherein the agent is an immunomodulatory agent.

13348. The method of item 13271 wherein the agent is sirolimus or an analogue or derivative thereof.

13349. The method of item 13271 wherein the agent is not sirolimus.

13350. The method of item 13271 wherein the agent is everolimus or an analogue or derivative thereof.

13351. The method of item 13271 wherein the agent is tacrolimus or an analogue or derivative thereof.

13352. The method of item 13271 wherein the agent is not tacrolimus.

13353. The method of item 13271 wherein the agent is biolmus or an analogue or derivative thereof.

13354. The method of item 13271 wherein the agent is tresperimus or an analogue or derivative thereof.

13355. The method of item 13271 wherein the agent is auranofin or an analogue or derivative thereof.

13356. The method of item 13271 wherein the agent is 27-0-demethylrapamycin or an analogue or derivative thereof.

13357. The method of item 13271 wherein the agent is gusperimus or an analogue or derivative thereof.

13358. The method of item 13271 wherein the agent is pimecrolimus or an analogue or derivative thereof.

13359. The method of item 13271 wherein the agent is ABT-578 or an analogue or derivative thereof.

13360. The method of item 13271 wherein the agent is an inosine monophosphate dehydrogenase (IMPDH) inhibitor.

13361. The method of item 13271 wherein the agent is an IMPDH inhibitor, wherein the IMPDH inhibitor is mycophenolic acid or an analogue or derivative thereof.

13362. The method of item 13271 wherein the agent is an IMPDH inhibitor, wherein the IMPDH inhibitor is 1-alpha-25 dihydroxy vitamin D₃ or an analogue or derivative thereof.

13363. The method of item 13271 wherein the agent is a leukotriene inhibitor.

13364. The method of item 13271 wherein the agent is a MCP-1 antagonist.

13365. The method of item 13271 wherein the agent is a MMP inhibitor.

13366. The method of item 13271 wherein the agent is an NF kappa B inhibitor.

13367. The method of item 13271 wherein the agent is an NF kappa B inhibitor, wherein the NF kappa B inhibitor is Bay 11-7082.

13368. The method of item 13271 wherein the agent is an NO agonist.

13369. The method of item 13271 wherein the agent is a p38 MAP kinase inhibitor.

13370. The method of item 13271 wherein the agent is a p38 MAP kinase inhibitor, wherein the p38 MAP kinase inhibitor is SB 202190.

13371. The method of item 13271 wherein the agent is a phosphodiesterase inhibitor.

13372. The method of item 13271 wherein the agent is a TGF beta inhibitor.

13373. The method of item 13271 wherein the agent is a thromboxane A2 antagonist.

13374. The method of item 13271 wherein the agent is a TNFa antagonist.

13375. The method of item 13271 wherein the agent is a TACE inhibitor.

13376. The method of item 13271 wherein the agent is a tyrosine kinase inhibitor.

13377. The method of item 13271 wherein the agent is a vitronectin inhibitor.

13378. The method of item 13271 wherein the agent is a fibroblast growth factor inhibitor.

13379. The method of item 13271 wherein the agent is a protein kinase inhibitor.

13380. The method of item 13271 wherein the agent is a PDGF receptor kinase inhibitor.

13381. The method of item 13271 wherein the agent is an endothelial growth factor receptor kinase inhibitor.

13382. The method of item 13271 wherein the agent is a retinoic acid receptor antagonist.

13383. The method of item 13271 wherein the agent is a platelet derived growth factor receptor kinase inhibitor.

13384. The method of item 13271 wherein the agent is a fibronogin antagonist.

13385. The method of item 13271 wherein the agent is an antimycotic agent.

13386. The method of item 13271 wherein the agent is an antimycotic agent, wherein the antimycotic agent is sulconizole.

13387. The method of item 13271 wherein the agent is a bisphosphonate.

13388. The method of item 13271 wherein the agent is a phospholipase A1 inhibitor.

13389. The method of item 13271 wherein the agent is a histamine H1/H2/H3 receptor antagonist.

13390. The method of item 13271 wherein the agent is a macrolide antibiotic.

13391. The method of item 13271 wherein the agent is a GPIIb/IIIa receptor antagonist.

13392. The method of item 13271 wherein the agent is an endothelin receptor antagonist.

13393. The method of item 13271 wherein the agent is a peroxisome proliferator-activated receptor agonist.

13394. The method of item 13271 wherein the agent is an estrogen receptor agent.

13395. The method of item 13271 wherein the agent is a somastostatin analogue.

13396. The method of item 13271 wherein the agent is a neurokinin 1 antagonist.

13397. The method of item 13271 wherein the agent is a neurokinin 3 antagonist.

13398. The method of item 13271 wherein the agent is a VLA-4 antagonist.

13399. The method of item 13271 wherein the agent is an osteoclast inhibitor.

13400. The method of item 13271 wherein the agent is a DNA topoisomerase ATP hydrolyzing inhibitor.

13401. The method of item 13271 wherein the agent is an angiotensin I converting enzyme inhibitor.

13402. The method of item 13271 wherein the agent is an angiotensin II antagonist.

13403. The method of item 13271 wherein the agent is an enkephalinase inhibitor.

13404. The method of item 13271 wherein the agent is a peroxisome proliferator-activated receptor gamma agonist insulin sensitizer.

13405. The method of item 13271 wherein the agent is a protein kinase C inhibitor.

13406. The method of item 13271 wherein the agent is a ROCK (rho-associated kinase) inhibitor.

13407. The method of item 13271 wherein the agent is a CXCR3 inhibitor.

13408. The method of item 13271 wherein the agent is an Itk inhibitor.

13409. The method of item 13271 wherein the agent is a cytosolic phospholipase A₂-alpha inhibitor.

13410. The method of item 13271 wherein the agent is a PPAR agonist.

13411. The method of item 13271 wherein the agent is an immunosuppressant.

13412. The method of item 13271 wherein the agent is an Erb inhibitor.

13413. The method of item 13271 wherein the agent is an apoptosis agonist.

13414. The method of item 13271 wherein the agent is a lipocortin agonist.

13415. The method of item 13271 wherein the agent is a VCAM-1 antagonist.

13416. The method of item 13271 wherein the agent is a collagen antagonist.

13417. The method of item 13271 wherein the agent is an alpha 2 integrin antagonist.

13418. The method of item 13271 wherein the agent is a TNF alpha inhibitor.

13419. The method of item 13271 wherein the agent is a nitric oxide inhibitor.

13420. The method of item 13271 wherein the agent is a cathepsin inhibitor.

13421. The method of item 13271 wherein the agent is not an anti-inflammatory agent.

13422. The method of item 13271 wherein the agent is not a steroid.

13423. The method of item 13271 wherein the agent is not a glucocorticosteroid.

13424. The method of item 13271 wherein the agent is not dexamethasone.

13425. The method of item 13271 wherein the agent is not an anti-infective agent.

13426. The method of item 13271 wherein the agent is not an antibiotic.

13427. The method of item 13271 wherein the agent is not an anti-fungal agent.

13428. The method of item 13271, wherein the composition comprises a polymer.

13429. The method of item 13271, wherein the composition comprises a polymeric carrier.

13430. The method of item 13271 wherein the anti-scarring agent inhibits adhesion between the device and a host into which the device is implanted.

13431. The method of item 13271 wherein the device delivers the anti-scarring agent locally to tissue proximate to the device.

13432. The method of item 13271 wherein the device has a coating that comprises the anti-scarring agent.

13433. The method of item 13271, wherein the device has a coating that comprises the agent and is disposed on a surface of the implant.

13434. The method of item 13271, wherein the device has a coating that comprises the agent and directly contacts the implant.

13435. The method of item 13271, wherein the device has a coating that comprises the agent and indirectly contacts the implant.

13436. The method of item 13271, wherein the device has a coating that comprises the agent and partially covers the implant.

13437. The method of item 13271, wherein the device has a coating that comprises the agent and completely covers the implant.

13438. The method of item 13271, wherein the device has a uniform coating.

13439. The method of item 13271, wherein the device has a non-uniform coating.

13440. The method of item 13271, wherein the device has a discontinuous coating.

13441. The method of item 13271, wherein the device has a patterned coating.

13442. The method of item 13271, wherein the device has a coating with a thickness of 100 μm or less.

13443. The method of item 13271, wherein the device has a coating with a thickness of 10 μm or less.

13444. The method of item 13271, wherein the device has a coating, and the coating adheres to the surface of the implant upon deployment of the implant.

13445. The method of item 13271, wherein the device has a coating, and wherein the coating is stable at room temperature for a period of 1 year.

13446. The method of item 13271, wherein the device has a coating, and wherein the anti-scarring agent is present in the coating in an amount ranging between about 0.0001% to about 1% by weight.

13447. The method of item 13271, wherein the device has a coating, and wherein the anti-scarring agent is present in the coating in an amount ranging between about 1% to about 10% by weight.

13448. The method of item 13271, wherein the device has a coating, and wherein the anti-scarring agent is present in the coating in an amount ranging between about 10% to about 25% by weight.

13449. The method of item 13271, wherein the device has a coating, and wherein the anti-scarring agent is present in the coating in an amount ranging between about 25% to about 70% by weight.

13450. The method of item 13271, wherein the device has a coating, and wherein the coating further comprises a polymer.

13451. The method of item 13271, wherein the device has a first coating having a first composition and a second coating having a second composition.

13452. The method of item 13271, wherein the device has a first coating having a first composition and a second coating having a second composition, wherein the first composition and the second composition are different.

13453. The method of item 13271, wherein the composition comprises a polymer.

13454. The method of item 13271, wherein the composition comprises a polymeric carrier.

13455. The method of item 13271, wherein the composition comprises a polymeric carrier, and wherein the polymeric carrier comprises a copolymer.

13456. The method of item 13271, wherein the composition comprises a polymeric carrier, and wherein the polymeric carrier comprises a block copolymer.

13457. The method of item 13271, wherein the composition comprises a polymeric carrier, and wherein the polymeric carrier comprises a random copolymer.

13458. The method of item 13271, wherein the composition comprises a polymeric carrier, and wherein the polymeric carrier comprises a biodegradable polymer.

13459. The method of item 13271, wherein the composition comprises a polymeric carrier, and wherein the polymeric carrier comprises a non-biodegradable polymer.

13460. The method of item 13271, wherein the composition comprises a polymeric carrier, and wherein the polymeric carrier comprises a hydrophilic polymer.

13461. The method of item 13271, wherein the composition comprises a polymeric carrier, and wherein the polymeric carrier comprises a hydrophobic polymer.

13462. The method of item 13271, wherein the composition comprises a polymeric carrier, and wherein the polymeric carrier comprises a polymer having hydrophilic domains.

13463. The method of item 13271, wherein the composition comprises a polymeric carrier, and wherein the polymeric carrier comprises a polymer having hydrophobic domains.

13464. The method of item 13271, wherein the composition comprises a polymeric carrier, and wherein the polymeric carrier comprises a non-conductive polymer.

13465. The method of item 13271, wherein the composition comprises a polymeric carrier, and wherein the polymeric carrier comprises an elastomer.

13466. The method of item 13271, wherein the composition comprises a polymeric carrier, and wherein the polymeric carrier comprises a hydrogel.

13467. The method of item 13271, wherein the composition comprises a polymeric carrier, and wherein the polymeric carrier comprises a silicone polymer.

13468. The method of item 13271, wherein the composition comprises a polymeric carrier, and wherein the polymeric carrier comprises a hydrocarbon polymer.

13469. The method of item 13271, wherein the composition comprises a polymeric carrier, and wherein the polymeric carrier comprises a styrene-derived polymer.

13470. The method of item 13271, wherein the composition comprises a polymeric carrier, and wherein the polymeric carrier comprises a butadiene polymer.

13471. The method of item 13271, wherein the composition comprises a polymeric carrier, and wherein the polymeric carrier comprises a macromer.

13472. The method of item 13271, wherein the composition comprises a polymeric carrier, and wherein the polymeric carrier comprises a poly(ethylene glycol)polymer.

13473. The method of item 13271 wherein the composition comprises a polymeric carrier, and wherein the polymeric carrier comprises an amorphous polymer.

13474. The method of item 13271, wherein the device comprises a lubricious coating.

13475. The method of item 13271 wherein the anti-scarring agent is located within pores or holes of the device.

13476. The method of item 13271 wherein the anti-scarring agent is located within a channel, lumen, or divet of the device.

13477. The method of item 13271, wherein the device comprises a second pharmaceutically active agent.

13478. The method of item 13271 wherein the device comprises an anti-inflammatory agent.

13479. The method of item 13271 wherein the device comprises an agent that inhibits infection.

13480. The method of item 13271 wherein the device comprises an agent that inhibits infection, and wherein the agent is an anthracycline.

13481. The method of item 13271 wherein the device comprises an agent that inhibits infection, and wherein the agent is doxorubicin.

13482. The method of item 13271 wherein the device comprises an agent that inhibits infection, and wherein the agent is mitoxantrone.

13483. The method of item 13271 wherein the device comprises an agent that inhibits infection, and wherein the agent is a fluoropyrimidine.

13484. The method of item 13271 wherein the device comprises an agent that inhibits infection, and wherein the agent is 5-fluorouracil (5-FU).

13485. The method of item 13271 wherein the device comprises an agent that inhibits infection, and wherein the agent is a folic acid antagonist.

13486. The method of item 13271 wherein the device comprises an agent that inhibits infection, and wherein the agent is methotrexate.

13487. The method of item 13271 wherein the device comprises an agent that inhibits infection, and wherein the agent is a podophylotoxin.

13488. The method of item 13271 wherein the device comprises an agent that inhibits infection, and wherein the agent is etoposide.

13489. The method of item 13271 wherein the device comprises an agent that inhibits infection, and wherein the agent is a camptothecin.

13490. The method of item 13271 wherein the device comprises an agent that inhibits infection, and wherein the agent is a hydroxyurea.

13491. The method of item 13271 wherein the device comprises an agent that inhibits infection, and wherein the agent is a platinum complex.

13492. The method of item 13271 wherein the device comprises an agent that inhibits infection, and wherein the agent is cisplatin.

13493. The method of item 13271, further comprising an anti-thrombotic agent.

13494. The method of item 13271 wherein the device comprises a visualization agent.

13495. The method of item 13271 wherein the device comprises a visualization agent, wherein the visualization agent is a radiopaque material, and wherein the radiopaque material comprises a metal, a halogenated compound, or a barium containing compound.

13496. The method of item 13271 wherein the device comprises a visualization agent, wherein the visualization agent is a radiopaque material, and wherein the radiopaque material comprises barium, tantalum, or technetium.

13497. The method of item 13271 wherein the device comprises a visualization agent, and wherein the visualization agent is a MRI responsive material.

13498. The method of item 13271 wherein the device comprises a visualization agent, and wherein the visualization agent comprises a gadolinium chelate.

13499. The method of item 13271 wherein the device comprises a visualization agent, and wherein the visualization agent comprises iron, magnesium, manganese, copper, or chromium.

13500. The method of item 13271 wherein the device comprises a visualization agent, and wherein the visualization agent comprises an iron oxide compound.

13501. The method of item 13271 wherein the device comprises a visualization agent, and wherein the visualization agent comprises a dye, pigment, or colorant.

13502. The method of item 13271 wherein the device comprises an echogenic material.

13503. The method of item 13271 wherein the device comprises an echogenic material, and wherein the echogenic material is in the form of a coating.

13504. The method of item 13271 wherein the device is sterile.

13505. The method of item 13271 wherein the anti-scarring agent is released into tissue in the vicinity of the device after deployment of the device.

13506. The method of item 13271 wherein the anti-scarring agent is released into tissue in the vicinity of the device after deployment of the device, and wherein the tissue is connective tissue.

13507. The method of item 13271 wherein the anti-scarring agent is released into tissue in the vicinity of the device after deployment of the device, and wherein the tissue is muscle tissue.

13508. The method of item 13271 wherein the anti-scarring agent is released into tissue in the vicinity of the device after deployment of the device, and wherein the tissue is nerve tissue.

13509. The method of item 13271 wherein the anti-scarring agent is released into tissue in the vicinity of the device after deployment of the device, and wherein the tissue is epithelium tissue.

13510. The method of item 13271 wherein the anti-scarring agent is released in effective concentrations from the device over a period ranging from the time of deployment of the device to about 1 year.

13511. The method of item 13271 wherein the anti-scarring agent is released in effective concentrations from the device over a period ranging from about 1 month to 6 months.

13512. The method of item 13271 wherein the anti-scarring agent is released in effective concentrations from the device over a period ranging from about 1-90 days.

13513. The method of item 13271 wherein the anti-scarring agent is released in effective concentrations from the device at a constant rate.

13514. The method of item 13271 wherein the anti-scarring agent is released in effective concentrations from the device at an increasing rate.

13515. The method of item 13271 wherein the anti-scarring agent is released in effective concentrations from the device at a decreasing rate.

13516. The method of item 13271 wherein the anti-scarring agent is released in effective concentrations from the composition comprising the anti-scarring agent by diffusion over a period ranging from the time of deployment of the device to about 90 days.

13517. The method of item 13271 wherein the anti-scarring agent is released in effective concentrations from the composition comprising the anti-scarring agent by erosion of the composition over a period ranging from the time of deployment of the device to about 90 days.

13518. The method of item 13271 wherein the device comprises about 0.01 μg to about 10 μg of the anti-scarring agent.

13519. The method of item 13271 wherein the device comprises about 10 μg to about 10 mg of the anti-scarring agent.

13520. The method of item 13271 wherein the device comprises about 10 mg to about 250 mg of the anti-scarring agent.

13521. The method of item 13271 wherein the device comprises about 250 mg to about 1000 mg of the anti-scarring agent.

13522. The method of item 13271 wherein the device comprises about 1000 mg to about 2500 mg of the anti-scarring agent.

13523. The method of item 13271 wherein a surface of the device comprises less than 0.01 μg of the anti-scarring agent per mm² of device surface to which the anti-scarring agent is applied.

13524. The method of item 13271 wherein a surface of the device comprises about 0.01 μg to about 1 μg of the anti-scarring agent per mm² of device surface to which the anti-scarring agent is applied.

13525. The method of item 13271 wherein a surface of the device comprises about 1 μg to about 10 μg of the anti-scarring agent per mm² of device surface to which the anti-scarring agent is applied.

13526. The method of item 13271 wherein a surface of the device comprises about 10 μg to about 250 μg of the anti-scarring agent per mm² of device surface to which the anti-scarring agent is applied.

13527. The method of item 13271 wherein a surface of the device comprises about 250 μg to about 1000 μg of the anti-scarring agent of anti-scarring agent per mm² of device surface to which the anti-scarring agent is applied.

13528. The method of item 13271 wherein a surface of the device comprises about 1000 μg to about 2500 μg of the anti-scarring agent per mm² of device surface to which the anti-scarring agent is applied.

13529. The method of item 13271 wherein the combining is performed by direct affixing the agent or the composition to the implant.

13530. The method of item 13271 wherein the combining is performed by spraying the agent or the component onto the implant.

13531. The method of item 13271 wherein the combining is performed by electrospraying the agent or the composition onto the implant.

13532. The method of item 13271 wherein the combining is performed by dipping the implant into a solution comprising the agent or the composition.

13533. The method of item 13271 wherein the combining is performed by covalently attaching the agent or the composition to the implant.

13534. The method of item 13271 wherein the combining is performed by non-covalently attaching the agent or the composition to the implant.

13535. The method of item 13271 wherein the combining is performed by coating the implant with a substance that contains the agent or the composition.

13536. The method of item 13271 wherein the combining is performed by coating the implant with a substance that absorbs the agent.

13537. The method of item 13271 wherein the combining is performed by interweaving a thread composed of, or coated with, the agent or the composition.

13538. The method of item 13271 wherein the combining is performed by covering all the implant with a sleeve that contains the agent or the composition.

13539. The method of item 13271 wherein the combining is performed by covering a portion of the implant with a sleeve that contains the agent or the composition.

13540. The method of item 13271 wherein the combining is performed by covering all the implant with a cover that contains the agent or the composition.

13541. The method of item 13271 wherein the combining is performed by covering a portion of the implant with a cover that contains the agent or the composition.

13542. The method of item 13271 wherein the combining is performed by covering all the implant with an electrospun fabric that contains the agent or the composition.

13543. The method of item 13271 wherein the combining is performed by covering a portion of the implant with an electrospun fabric that contains the agent or the composition.

13544. The method of item 13271 wherein the combining is performed by covering all the implant with a mesh that contains the agent or the composition.

13545. The method of item 13271 wherein the combining is performed by covering a portion of the implant with a mesh that contains the agent or the composition.

13546. The method of item 13271 wherein the combining is performed by constructing all the implant with the agent or the composition.

13547. The method of item 13271 wherein the combining is performed by constructing a portion of the implant with the agent or the composition.

13548. The method of item 13271 wherein the combining is performed by impregnating the implant with the agent or the composition.

13549. The method of item 13271 wherein the combining is performed by constructing all of the implant from a degradable polymer that releases the agent.

13550. The method of item 13271 wherein the combining is performed by constructing a portion of the implant from a degradable polymer that releases the agent.

13551. The method of item 13271 wherein the combining is performed by dipping the implant into a solution that comprise the agent and an inert solvent for the implant.

13552. The method of item 13271 wherein the combining is performed by dipping the implant into a solution that comprises the agent and a solvent that will swill the implant.

13553. The method of item 13271 wherein the combining is performed by dipping the implant into a solution that comprises the agent and a solvent that will dissolve the implant.

13554. The method of item 13271 wherein the combining is performed by dipping the implant into a solution that comprises the agent, a polymer and an inert solvent for the implant.

13555. The method of item 13271 wherein the combining is performed by dipping the implant into a solution that comprises the agent, a polymer and a solvent that will swill the implant.

13556. The method of item 13271 wherein the combining is performed by dipping the implant into a solution that comprises the agent, a polymer and a solvent that will dissolve the implant.

13557. The method of item 13271 wherein the combining is performed by spraying the implant into a solution that comprises the agent and an inert solvent for the implant.

13558. The method of item 13271 wherein the combining is performed by spraying the implant into a solution that comprises the agent and a solvent that will swill the implant.

13559. The method of item 13271 wherein the combining is performed by spraying the implant into a solution that comprises the agent and a solvent that will dissolve the implant.

13560. The method of item 13271 wherein the combining is performed by spraying the implant into a solution that comprises the agent, a polymer and an inert solvent for the implant.

13561. The method of item 13271 wherein the combining is performed by spraying the implant into a solution that comprises the agent, a polymer and a solvent that will swill the implant.

13562. The method of item 13271 wherein the combining is performed by spraying the implant into a solution that comprises the agent, a polymer and a solvent that will dissolve the implant.

13563. The method of item 13271 wherein the implant is a ventriculopleural shunt.

13564. The method of item 13271 wherein the implant is a jugular vein shunt.

13565. The method of item 13271 wherein the implant is a vena cava (VA) shunt.

13566. The method of item 13271 wherein the implant is a ventriculoperitoneal shunt (VP shunt).

13567. The method of item 13271 wherein the implant is a gallbladder shunt.

13568. The method of item 13271 wherein the implant is a peritoneum shunt.

13569. The method of item 13271 wherein the implant is an external ventricular drainage (EVD) device.

13570. The method of item 13271 wherein the implant is an intracranial pressure (ICP) monitoring device.

13571. The method of item 13271 wherein the implant is a dural patch to prevent epidural fibrosis post-laminectomy.

13572. The method of item 13271 wherein the implant is a device for continuous subarachnoid infusions.

13573. A method of making a medical device comprising: combining an intraocular lens (i.e., an implant) and an anti-scarring agent or a composition comprising an anti-scarring agent, wherein the agent inhibits scarring between the device and a host into which the device is implanted.

13574. The method of item 13573 wherein the agent inhibits cell regeneration.

13575. The method of item 13573 wherein the agent inhibits angiogenesis.

13576. The method of item 13573 wherein the agent inhibits fibroblast migration.

13577. The method of item 13573 wherein the agent inhibits fibroblast proliferation.

13578. The method of item 13573 wherein the agent inhibits deposition of extracellular matrix.

13579. The method of item 13573 wherein the agent inhibits tissue remodeling.

13580. The method of item 13573 wherein the agent is an angiogenesis inhibitor.

13581. The method of item 13573 wherein the agent is a 5-lipoxygenase inhibitor or antagonist.

13582. The method of item 13573 wherein the agent is a chemokine receptor antagonist.

13583. The method of item 13573 wherein the agent is a cell cycle inhibitor.

13584. The method of item 13573 wherein the agent is a taxane.

13585. The method of item 13573 wherein the agent is an anti-microtubule agent.

13586. The method of item 13573 wherein the agent is paclitaxel.

13587. The method of item 13573 wherein the agent is not paclitaxel.

13588. The method of item 13573 wherein the agent is an analogue or derivative of paclitaxel.

13589. The method of item 13573 wherein the agent is a vinca alkaloid.

13590. The method of item 13573 wherein the agent is camptothecin or an analogue or derivative thereof.

13591. The method of item 13573 wherein the agent is a podophyllotoxin.

13592. The method of item 13573 wherein the agent is a podophyllotoxin, wherein the podophyllotoxin is etoposide or an analogue or derivative thereof.

13593. The method of item 13573 wherein the agent is an anthracycline.

13594. The method of item 13573 wherein the agent is an anthracycline, wherein the anthracycline is doxorubicin or an analogue or derivative thereof.

13595. The method of item 13573 wherein the agent is an anthracycline, wherein the anthracycline is mitoxantrone or an analogue or derivative thereof.

13596. The method of item 13573 wherein the agent is a platinum compound.

13597. The method of item 13573 wherein the agent is a nitrosourea.

13598. The method of item 13573 wherein the agent is a nitroimidazole.

13599. The method of item 13573 wherein the agent is a folic acid antagonist.

13600. The method of item 13573 wherein the agent is a cytidine analogue.

13601. The method of item 13573 wherein the agent is a pyrimidine analogue.

13602. The method of item 13573 wherein the agent is a fluoropyrimidine analogue.

13603. The method of item 13573 wherein the agent is a purine analogue.

13604. The method of item 13573 wherein the agent is a nitrogen mustard or an analogue or derivative thereof.

13605. The method of item 13573 wherein the agent is a hydroxyurea.

13606. The method of item 13573 wherein the agent is a mytomicin or an analogue or derivative thereof.

13607. The method of item 13573 wherein the agent is an alkyl sulfonate.

13608. The method of item 13573 wherein the agent is a benzamide or an analogue or derivative thereof.

13609. The method of item 13573 wherein the agent is a nicotinamide or an analogue or derivative thereof.

13610. The method of item 13573 wherein the agent is a halogenated sugar or an analogue or derivative thereof.

13611. The method of item 13573 wherein the agent is a DNA alkylating agent.

13612. The method of item 13573 wherein the agent is an anti-microtubule agent.

13613. The method of item 13573 wherein the agent is a topoisomerase inhibitor.

13614. The method of item 13573 wherein the agent is a DNA cleaving agent.

13615. The method of item 13573 wherein the agent is an antimetabolite.

13616. The method of item 13573 wherein the agent inhibits adenosine deaminase.

13617. The method of item 13573 wherein the agent inhibits purine ring synthesis.

13618. The method of item 13573 wherein the agent is a nucleotide interconversion inhibitor.

13619. The method of item 13573 wherein the agent inhibits dihydrofolate reduction.

13620. The method of item 13573 wherein the agent blocks thymidine monophosphate.

13621. The method of item 13573 wherein the agent causes DNA damage.

13622. The method of item 13573 wherein the agent is a DNA intercalation agent.

13623. The method of item 13573 wherein the agent is a RNA synthesis inhibitor.

13624. The method of item 13573 wherein the agent is a pyrimidine synthesis inhibitor.

13625. The method of item 13573 wherein the agent inhibits ribonucleotide synthesis or function.

13626. The method of item 13573 wherein the agent inhibits thymidine monophosphate synthesis or function.

13627. The method of item 13573 wherein the agent inhibits DNA synthesis.

13628. The method of item 13573 wherein the agent causes DNA adduct formation.

13629. The method of item 13573 wherein the agent inhibits protein synthesis.

13630. The method of item 13573 wherein the agent inhibits microtubule function.

13631. The method of item 13573 wherein the agent is a cyclin dependent protein kinase inhibitor.

13632. The method of item 13573 wherein the agent is an epidermal growth factor kinase inhibitor.

13633. The method of item 13573 wherein the agent is an elastase inhibitor.

13634. The method of item 13573 wherein the agent is a factor Xa inhibitor.

13635. The method of item 13573 wherein the agent is a farnesyltransferase inhibitor.

13636. The method of item 13573 wherein the agent is a fibrinogen antagonist.

13637. The method of item 13573 wherein the agent is a guanylate cyclase stimulant.

13638. The method of item 13573 wherein the agent is a heat shock protein 90 antagonist.

13639. The method of item 13573 wherein the agent is a heat shock protein 90 antagonist, wherein the heat shock protein 90 antagonist is geldanamycin or an analogue or derivative thereof.

13640. The method of item 13573 wherein the agent is a guanylate cyclase stimulant.

13641. The method of item 13573 wherein the agent is a HMGCoA reductase inhibitor.

13642. The method of item 13573 wherein the agent is a HMGCoA reductase inhibitor, wherein the HMGCoA reductase inhibitor is simvastatin or an analogue or derivative thereof.

13643. The method of item 13573 wherein the agent is a hydroorotate dehydrogenase inhibitor.

13644. The method of item 13573 wherein the agent is an IKK2 inhibitor.

13645. The method of item 13573 wherein the agent is an IL-1 antagonist.

13646. The method of item 13573 wherein the agent is an ICE antagonist.

13647. The method of item 13573 wherein the agent is an IRAK antagonist.

13648. The method of item 13573 wherein the agent is an IL-4 agonist.

13649. The method of item 13573 wherein the agent is an immunomodulatory agent.

13650. The method of item 13573 wherein the agent is sirolimus or an analogue or derivative thereof.

13651. The method of item 13573 wherein the agent is not sirolimus.

13652. The method of item 13573 wherein the agent is everolimus or an analogue or derivative thereof.

13653. The method of item 13573 wherein the agent is tacrolimus or an analogue or derivative thereof.

13654. The method of item 13573 wherein the agent is not tacrolimus.

13655. The method of item 13573 wherein the agent is biolmus or an analogue or derivative thereof.

13656. The method of item 13573 wherein the agent is tresperimus or an analogue or derivative thereof.

13657. The method of item 13573 wherein the agent is auranofin or an analogue or derivative thereof.

13658. The method of item 13573 wherein the agent is 27-0-demethylrapamycin or an analogue or derivative thereof.

13659. The method of item 13573 wherein the agent is gusperimus or an analogue or derivative thereof.

13660. The method of item 13573 wherein the agent is pimecrolimus or an analogue or derivative thereof.

13661. The method of item 13573 wherein the agent is ABT-578 or an analogue or derivative thereof.

13662. The method of item 13573 wherein the agent is an inosine monophosphate dehydrogenase (IMPDH) inhibitor.

13663. The method of item 13573 wherein the agent is an IMPDH inhibitor, wherein the IMPDH inhibitor is mycophenolic acid or an analogue or derivative thereof.

13664. The method of item 13573 wherein the agent is an IMPDH inhibitor, wherein the IMPDH inhibitor is 1-alpha-25 dihydroxy vitamin D₃ or an analogue or derivative thereof.

13665. The method of item 13573 wherein the agent is a leukotriene inhibitor.

13666. The method of item 13573 wherein the agent is a MCP-1 antagonist.

13667. The method of item 13573 wherein the agent is a MMP inhibitor.

13668. The method of item 13573 wherein the agent is an NF kappa B inhibitor.

13669. The method of item 13573 wherein the agent is an NF kappa B inhibitor, wherein the NF kappa B inhibitor is Bay 11-7082.

13670. The method of item 13573 wherein the agent is an NO agonist.

13671. The method of item 13573 wherein the agent is a p38 MAP kinase inhibitor.

13672. The method of item 13573 wherein the agent is a p38 MAP kinase inhibitor, wherein the p38 MAP kinase inhibitor is SB 202190.

13673. The method of item 13573 wherein the agent is a phosphodiesterase inhibitor.

13674. The method of item 13573 wherein the agent is a TGF beta inhibitor.

13675. The method of item 13573 wherein the agent is a thromboxane A2 antagonist.

13676. The method of item 13573 wherein the agent is a TNFa antagonist.

13677. The method of item 13573 wherein the agent is a TACE inhibitor.

13678. The method of item 13573 wherein the agent is a tyrosine kinase inhibitor.

13679. The method of item 13573 wherein the agent is a vitronectin inhibitor.

13680. The method of item 13573 wherein the agent is a fibroblast growth factor inhibitor.

13681. The method of item 13573 wherein the agent is a protein kinase inhibitor.

13682. The method of item 13573 wherein the agent is a PDGF receptor kinase inhibitor.

13683. The method of item 13573 wherein the agent is an endothelial growth factor receptor kinase inhibitor.

13684. The method of item 13573 wherein the agent is a retinoic acid receptor antagonist.

13685. The method of item 13573 wherein the agent is a platelet derived growth factor receptor kinase inhibitor.

13686. The method of item 13573 wherein the agent is a fibronogin antagonist.

13687. The method of item 13573 wherein the agent is an antimycotic agent.

13688. The method of item 13573 wherein the agent is an antimycotic agent, wherein the antimycotic agent is sulconizole.

13689. The method of item 13573 wherein the agent is a bisphosphonate.

13690. The method of item 13573 wherein the agent is a phospholipase A1 inhibitor.

13691. The method of item 13573 wherein the agent is a histamine H1/H2/H3 receptor antagonist.

13692. The method of item 13573 wherein the agent is a macrolide antibiotic.

13693. The method of item 13573 wherein the agent is a GPIIb/IIIa receptor antagonist.

13694. The method of item 13573 wherein the agent is an endothelin receptor antagonist.

13695. The method of item 13573 wherein the agent is a peroxisome proliferator-activated receptor agonist.

13696. The method of item 13573 wherein the agent is an estrogen receptor agent.

13697. The method of item 13573 wherein the agent is a somastostatin analogue.

13698. The method of item 13573 wherein the agent is a neurokinin 1 antagonist.

13699. The method of item 13573 wherein the agent is a neurokinin 3 antagonist.

13700. The method of item 13573 wherein the agent is a VLA-4 antagonist.

13701. The method of item 13573 wherein the agent is an osteoclast inhibitor.

13702. The method of item 13573 wherein the agent is a DNA topoisomerase ATP hydrolyzing inhibitor.

13703. The method of item 13573 wherein the agent is an angiotensin I converting enzyme inhibitor.

13704. The method of item 13573 wherein the agent is an angiotensin II antagonist.

13705. The method of item 13573 wherein the agent is an enkephalinase inhibitor.

13706. The method of item 13573 wherein the agent is a peroxisome proliferator-activated receptor gamma agonist insulin sensitizer.

13707. The method of item 13573 wherein the agent is a protein kinase C inhibitor.

13708. The method of item 13573 wherein the agent is a ROCK (rho-associated kinase) inhibitor.

13709. The method of item 13573 wherein the agent is a CXCR3 inhibitor.

13710. The method of item 13573 wherein the agent is an Itk inhibitor.

13711. The method of item 13573 wherein the agent is a cytosolic phospholipase A₂-alpha inhibitor.

13712. The method of item 13573 wherein the agent is a PPAR agonist.

13713. The method of item 13573 wherein the agent is an immunosuppressant.

13714. The method of item 13573 wherein the agent is an Erb inhibitor.

13715. The method of item 13573 wherein the agent is an apoptosis agonist.

13716. The method of item 13573 wherein the agent is a lipocortin agonist.

13717. The method of item 13573 wherein the agent is a VCAM-1 antagonist.

13718. The method of item 13573 wherein the agent is a collagen antagonist.

13719. The method of item 13573 wherein the agent is an alpha 2 integrin antagonist.

13720. The method of item 13573 wherein the agent is a TNF alpha inhibitor.

13721. The method of item 13573 wherein the agent is a nitric oxide inhibitor.

13722. The method of item 13573 wherein the agent is a cathepsin inhibitor.

13723. The method of item 13573 wherein the agent is not an anti-inflammatory agent.

13724. The method of item 13573 wherein the agent is not a steroid.

13725. The method of item 13573 wherein the agent is not a glucocorticosteroid.

13726. The method of item 13573 wherein the agent is not dexamethasone.

13727. The method of item 13573 wherein the agent is not an anti-infective agent.

13728. The method of item 13573 wherein the agent is not an antibiotic.

13729. The method of item 13573 wherein the agent is not an anti-fungal agent.

13730. The method of item 13573, wherein the composition comprises a polymer.

13731. The method of item 13573, wherein the composition comprises a polymeric carrier.

13732. The method of item 13573 wherein the anti-scarring agent inhibits adhesion between the device and a host into which the device is implanted.

13733. The method of item 13573 wherein the device delivers the anti-scarring agent locally to tissue proximate to the device.

13734. The method of item 13573 wherein the device has a coating that comprises the anti-scarring agent.

13735. The method of item 13573, wherein the device has a coating that comprises the agent and is disposed on a surface of the implant.

13736. The method of item 13573, wherein the device has a coating that comprises the agent and directly contacts the implant.

13737. The method of item 13573, wherein the device has a coating that comprises the agent and indirectly contacts the implant.

13738. The method of item 13573, wherein the device has a coating that comprises the agent and partially covers the implant.

13739. The method of item 13573, wherein the device has a coating that comprises the agent and completely covers the implant.

13740. The method of item 13573, wherein the device has a uniform coating.

13741. The method of item 13573, wherein the device has a non-uniform coating.

13742. The method of item 13573, wherein the device has a discontinuous coating.

13743. The method of item 13573, wherein the device has a patterned coating.

13744. The method of item 13573, wherein the device has a coating with a thickness of 100 μm or less.

13745. The method of item 13573, wherein the device has a coating with a thickness of 10 μm or less.

13746. The method of item 13573, wherein the device has a coating, and the coating adheres to the surface of the implant upon deployment of the implant.

13747. The method of item 13573, wherein the device has a coating, and wherein the coating is stable at room temperature for a period of 1 year.

13748. The method of item 13573, wherein the device has a coating, and wherein the anti-scarring agent is present in the coating in an amount ranging between about 0.0001% to about 1% by weight.

13749. The method of item 13573, wherein the device has a coating, and wherein the anti-scarring agent is present in the coating in an amount ranging between about 1% to about 10% by weight.

13750. The method of item 13573, wherein the device has a coating, and wherein the anti-scarring agent is present in the coating in an amount ranging between about 10% to about 25% by weight.

13751. The method of item 13573, wherein the device has a coating, and wherein the anti-scarring agent is present in the coating in an amount ranging between about 25% to about 70% by weight.

13752. The method of item 13573, wherein the device has a coating, and wherein the coating further comprises a polymer.

13753. The method of item 13573, wherein the device has a first coating having a first composition and a second coating having a second composition.

13754. The method of item 13573, wherein the device has a first coating having a first composition and a second coating having a second composition, wherein the first composition and the second composition are different.

13755. The method of item 13573, wherein the composition comprises a polymer.

13756. The method of item 13573, wherein the composition comprises a polymeric carrier.

13757. The method of item 13573, wherein the composition comprises a polymeric carrier, and wherein the polymeric carrier comprises a copolymer.

13758. The method of item 13573, wherein the composition comprises a polymeric carrier, and wherein the polymeric carrier comprises a block copolymer.

13759. The method of item 13573, wherein the composition comprises a polymeric carrier, and wherein the polymeric carrier comprises a random copolymer.

13760. The method of item 13573, wherein the composition comprises a polymeric carrier, and wherein the polymeric carrier comprises a biodegradable polymer.

13761. The method of item 13573, wherein the composition comprises a polymeric carrier, and wherein the polymeric carrier comprises a non-biodegradable polymer.

13762. The method of item 13573, wherein the composition comprises a polymeric carrier, and wherein the polymeric carrier comprises a hydrophilic polymer.

13763. The method of item 13573, wherein the composition comprises a polymeric carrier, and wherein the polymeric carrier comprises a hydrophobic polymer.

13764. The method of item 13573, wherein the composition comprises a polymeric carrier, and wherein the polymeric carrier comprises a polymer having hydrophilic domains.

13765. The method of item 13573, wherein the composition comprises a polymeric carrier, and wherein the polymeric carrier comprises a polymer having hydrophobic domains.

13766. The method of item 13573, wherein the composition comprises a polymeric carrier, and wherein the polymeric carrier comprises a non-conductive polymer.

13767. The method of item 13573, wherein the composition comprises a polymeric carrier, and wherein the polymeric carrier comprises an elastomer.

13768. The method of item 13573, wherein the composition comprises a polymeric carrier, and wherein the polymeric carrier comprises a hydrogel.

13769. The method of item 13573, wherein the composition comprises a polymeric carrier, and wherein the polymeric carrier comprises a silicone polymer.

13770. The method of item 13573, wherein the composition comprises a polymeric carrier, and wherein the polymeric carrier comprises a hydrocarbon polymer.

13771. The method of item 13573, wherein the composition comprises a polymeric carrier, and wherein the polymeric carrier comprises a styrene-derived polymer.

13772. The method of item 13573, wherein the composition comprises a polymeric carrier, and wherein the polymeric carrier comprises a butadiene polymer.

13773. The method of item 13573, wherein the composition comprises a polymeric carrier, and wherein the polymeric carrier comprises a macromer.

13774. The method of item 13573, wherein the composition comprises a polymeric carrier, and wherein the polymeric carrier comprises a poly(ethylene glycol)polymer.

13775. The method of item 13573 wherein the composition comprises a polymeric carrier, and wherein the polymeric carrier comprises an amorphous polymer.

13776. The method of item 13573, wherein the device comprises a lubricious coating.

13777. The method of item 13573 wherein the anti-scarring agent is located within pores or holes of the device.

13778. The method of item 13573 wherein the anti-scarring agent is located within a channel, lumen, or divet of the device.

13779. The method of item 13573, wherein the device comprises a second pharmaceutically active agent.

13780. The method of item 13573 wherein the device comprises an anti-inflammatory agent.

13781. The method of item 13573 wherein the device comprises an agent that inhibits infection.

13782. The method of item 13573 wherein the device comprises an agent that inhibits infection, and wherein the agent is an anthracycline.

13783. The method of item 13573 wherein the device comprises an agent that inhibits infection, and wherein the agent is doxorubicin.

13784. The method of item 13573 wherein the device comprises an agent that inhibits infection, and wherein the agent is mitoxantrone.

13785. The method of item 13573 wherein the device comprises an agent that inhibits infection, and wherein the agent is a fluoropyrimidine.

13786. The method of item 13573 wherein the device comprises an agent that inhibits infection, and wherein the agent is 5-fluorouracil (5-FU).

13787. The method of item 13573 wherein the device comprises an agent that inhibits infection, and wherein the agent is a folic acid antagonist.

13788. The method of item 13573 wherein the device comprises an agent that inhibits infection, and wherein the agent is methotrexate.

13789. The method of item 13573 wherein the device comprises an agent that inhibits infection, and wherein the agent is a podophylotoxin.

13790. The method of item 13573 wherein the device comprises an agent that inhibits infection, and wherein the agent is etoposide.

13791. The method of item 13573 wherein the device comprises an agent that inhibits infection, and wherein the agent is a camptothecin.

13792. The method of item 13573 wherein the device comprises an agent that inhibits infection, and wherein the agent is a hydroxyurea.

13793. The method of item 13573 wherein the device comprises an agent that inhibits infection, and wherein the agent is a platinum complex.

13794. The method of item 13573 wherein the device comprises an agent that inhibits infection, and wherein the agent is cisplatin.

13795. The method of item 13573, further comprising an anti-thrombotic agent.

13796. The method of item 13573 wherein the device comprises a visualization agent.

13797. The method of item 13573 wherein the device comprises a visualization agent, wherein the visualization agent is a radiopaque material, and wherein the radiopaque material comprises a metal, a halogenated compound, or a barium containing compound.

13798. The method of item 13573 wherein the device comprises a visualization agent, wherein the visualization agent is a radiopaque material, and wherein the radiopaque material comprises barium, tantalum, or technetium.

13799. The method of item 13573 wherein the device comprises a visualization agent, and wherein the visualization agent is a MRI responsive material.

13800. The method of item 13573 wherein the device comprises a visualization agent, and wherein the visualization agent comprises a gadolinium chelate.

13801. The method of item 13573 wherein the device comprises a visualization agent, and wherein the visualization agent comprises iron, magnesium, manganese, copper, or chromium.

13802. The method of item 13573 wherein the device comprises a visualization agent, and wherein the visualization agent comprises an iron oxide compound.

13803. The method of item 13573 wherein the device comprises a visualization agent, and wherein the visualization agent comprises a dye, pigment, or colorant.

13804. The method of item 13573 wherein the device comprises an echogenic material.

13805. The method of item 13573 wherein the device comprises an echogenic material, and wherein the echogenic material is in the form of a coating.

13806. The method of item 13573 wherein the device is sterile.

13807. The method of item 13573 wherein the anti-scarring agent is released into tissue in the vicinity of the device after deployment of the device.

13808. The method of item 13573 wherein the anti-scarring agent is released into tissue in the vicinity of the device after deployment of the device, and wherein the tissue is connective tissue.

13809. The method of item 13573 wherein the anti-scarring agent is released into tissue in the vicinity of the device after deployment of the device, and wherein the tissue is muscle tissue.

13810. The method of item 13573 wherein the anti-scarring agent is released into tissue in the vicinity of the device after deployment of the device, and wherein the tissue is nerve tissue.

13811. The method of item 13573 wherein the anti-scarring agent is released into tissue in the vicinity of the device after deployment of the device, and wherein the tissue is epithelium tissue.

13812. The method of item 13573 wherein the anti-scarring agent is released in effective concentrations from the device over a period ranging from the time of deployment of the device to about 1 year.

13813. The method of item 13573 wherein the anti-scarring agent is released in effective concentrations from the device over a period ranging from about 1 month to 6 months.

13814. The method of item 13573 wherein the anti-scarring agent is released in effective concentrations from the device over a period ranging from about 1-90 days.

13815. The method of item 13573 wherein the anti-scarring agent is released in effective concentrations from the device at a constant rate.

13816. The method of item 13573 wherein the anti-scarring agent is released in effective concentrations from the device at an increasing rate.

13817. The method of item 13573 wherein the anti-scarring agent is released in effective concentrations from the device at a decreasing rate.

13818. The method of item 13573 wherein the anti-scarring agent is released in effective concentrations from the composition comprising the anti-scarring agent by diffusion over a period ranging from the time of deployment of the device to about 90 days.

13819. The method of item 13573 wherein the anti-scarring agent is released in effective concentrations from the composition comprising the anti-scarring agent by erosion of the composition over a period ranging from the time of deployment of the device to about 90 days.

13820. The method of item 13573 wherein the device comprises about 0.01 μg to about 10 μg of the anti-scarring agent.

13821. The method of item 13573 wherein the device comprises about 10 μg to about 10 mg of the anti-scarring agent.

13822. The method of item 13573 wherein the device comprises about 10 mg to about 250 mg of the anti-scarring agent.

13823. The method of item 13573 wherein the device comprises about 250 mg to about 1000 mg of the anti-scarring agent.

13824. The method of item 13573 wherein the device comprises about 1000 mg to about 2500 mg of the anti-scarring agent.

13825. The method of item 13573 wherein a surface of the device comprises less than 0.01 μg of the anti-scarring agent per mm² of device surface to which the anti-scarring agent is applied.

13826. The method of item 13573 wherein a surface of the device comprises about 0.01 μg to about 1 μg of the anti-scarring agent per mm² of device surface to which the anti-scarring agent is applied.

13827. The method of item 13573 wherein a surface of the device comprises about 1 μg to about 10 μg of the anti-scarring agent per mm² of device surface to which the anti-scarring agent is applied.

13828. The method of item 13573 wherein a surface of the device comprises about 10 μg to about 250 μg of the anti-scarring agent per mm² of device surface to which the anti-scarring agent is applied.

13829. The method of item 13573 wherein a surface of the device comprises about 250 μg to about 1000 μg of the anti-scarring agent of anti-scarring agent per mm² of device surface to which the anti-scarring agent is applied.

13830. The method of item 13573 wherein a surface of the device comprises about 1000 μg to about 2500 μg of the anti-scarring agent per mm² of device surface to which the anti-scarring agent is applied.

13831. The method of item 13573 wherein the combining is performed by direct affixing the agent or the composition to the implant.

13832. The method of item 13573 wherein the combining is performed by spraying the agent or the component onto the implant.

13833. The method of item 13573 wherein the combining is performed by electrospraying the agent or the composition onto the implant.

13834. The method of item 13573 wherein the combining is performed by dipping the implant into a solution comprising the agent or the composition.

13835. The method of item 13573 wherein the combining is performed by covalently attaching the agent or the composition to the implant.

13836. The method of item 13573 wherein the combining is performed by non-covalently attaching the agent or the composition to the implant.

13837. The method of item 13573 wherein the combining is performed by coating the implant with a substance that contains the agent or the composition.

13838. The method of item 13573 wherein the combining is performed by coating the implant with a substance that absorbs the agent.

13839. The method of item 13573 wherein the combining is performed by interweaving a thread composed of, or coated with, the agent or the composition.

13840. The method of item 13573 wherein the combining is performed by covering all the implant with a sleeve that contains the agent or the composition.

13841. The method of item 13573 wherein the combining is performed by covering a portion of the implant with a sleeve that contains the agent or the composition.

13842. The method of item 13573 wherein the combining is performed by covering all the implant with a cover that contains the agent or the composition.

13843. The method of item 13573 wherein the combining is performed by covering a portion of the implant with a cover that contains the agent or the composition.

13844. The method of item 13573 wherein the combining is performed by covering all the implant with an electrospun fabric that contains the agent or the composition.

13845. The method of item 13573 wherein the combining is performed by covering a portion of the implant with an electrospun fabric that contains the agent or the composition.

13846. The method of item 13573 wherein the combining is performed by covering all the implant with a mesh that contains the agent or the composition.

13847. The method of item 13573 wherein the combining is performed by covering a portion of the implant with a mesh that contains the agent or the composition.

13848. The method of item 13573 wherein the combining is performed by constructing all the implant with the agent or the composition.

13849. The method of item 13573 wherein the combining is performed by constructing a portion of the implant with the agent or the composition.

13850. The method of item 13573 wherein the combining is performed by impregnating the implant with the agent or the composition.

13851. The method of item 13573 wherein the combining is performed by constructing all of the implant from a degradable polymer that releases the agent.

13852. The method of item 13573 wherein the combining is performed by constructing a portion of the implant from a degradable polymer that releases the agent.

13853. The method of item 13573 wherein the combining is performed by dipping the implant into a solution that comprise the agent and an inert solvent for the implant.

13854. The method of item 13573 wherein the combining is performed by dipping the implant into a solution that comprises the agent and a solvent that will swill the implant.

13855. The method of item 13573 wherein the combining is performed by dipping the implant into a solution that comprises the agent and a solvent that will dissolve the implant.

13856. The method of item 13573 wherein the combining is performed by dipping the implant into a solution that comprises the agent, a polymer and an inert solvent for the implant.

13857. The method of item 13573 wherein the combining is performed by dipping the implant into a solution that comprises the agent, a polymer and a solvent that will swill the implant.

13858. The method of item 13573 wherein the combining is performed by dipping the implant into a solution that comprises the agent, a polymer and a solvent that will dissolve the implant.

13859. The method of item 13573 wherein the combining is performed by spraying the implant into a solution that comprises the agent and an inert solvent for the implant.

13860. The method of item 13573 wherein the combining is performed by spraying the implant into a solution that comprises the agent and a solvent that will swill the implant.

13861. The method of item 13573 wherein the combining is performed by spraying the implant into a solution that comprises the agent and a solvent that will dissolve the implant.

13862. The method of item 13573 wherein the combining is performed by spraying the implant into a solution that comprises the agent, a polymer and an inert solvent for the implant.

13863. The method of item 13573 wherein the combining is performed by spraying the implant into a solution that comprises the agent, a polymer and a solvent that will swill the implant.

13864. The method of item 13573 wherein the combining is performed by spraying the implant into a solution that comprises the agent, a polymer and a solvent that will dissolve the implant.

13865. The method of item 13573 wherein the implant is an aphakic lens.

13866. The method of item 13573 wherein the implant is a phakic lens.

13867. The method of item 13573 wherein the implant is a multi-focal lens.

13868. A method of making a medical device comprising: combining a glaucoma drainage device (i.e., an implant) and an anti-scarring agent or a composition comprising an anti-scarring agent, wherein the agent inhibits scarring between the device and a host into which the device is implanted.

13869. The method of item 13868 wherein the agent inhibits cell regeneration.

13870. The method of item 13868 wherein the agent inhibits angiogenesis.

13871. The method of item 13868 wherein the agent inhibits fibroblast migration.

13872. The method of item 13868 wherein the agent inhibits fibroblast proliferation.

13873. The method of item 13868 wherein the agent inhibits deposition of extracellular matrix.

13874. The method of item 13868 wherein the agent inhibits tissue remodeling.

13875. The method of item 13868 wherein the agent is an angiogenesis inhibitor.

13876. The method of item 13868 wherein the agent is a 5-lipoxygenase inhibitor or antagonist.

13877. The method of item 13868 wherein the agent is a chemokine receptor antagonist.

13878. The method of item 13868 wherein the agent is a cell cycle inhibitor.

13879. The method of item 13868 wherein the agent is a taxane.

13880. The method of item 13868 wherein the agent is an anti-microtubule agent.

13881. The method of item 13868 wherein the agent is paclitaxel.

13882. The method of item 13868 wherein the agent is not paclitaxel.

13883. The method of item 13868 wherein the agent is an analogue or derivative of paclitaxel.

13884. The method of item 13868 wherein the agent is a vinca alkaloid.

13885. The method of item 13868 wherein the agent is camptothecin or an analogue or derivative thereof.

13886. The method of item 13868 wherein the agent is a podophyllotoxin.

13887. The method of item 13868 wherein the agent is a podophyllotoxin, wherein the podophyllotoxin is etoposide or an analogue or derivative thereof.

13888. The method of item 13868 wherein the agent is an anthracycline.

13889. The method of item 13868 wherein the agent is an anthracycline, wherein the anthracycline is doxorubicin or an analogue or derivative thereof.

13890. The method of item 13868 wherein the agent is an anthracycline, wherein the anthracycline is mitoxantrone or an analogue or derivative thereof.

13891. The method of item 13868 wherein the agent is a platinum compound.

13892. The method of item 13868 wherein the agent is a nitrosourea.

13893. The method of item 13868 wherein the agent is a nitroimidazole.

13894. The method of item 13868 wherein the agent is a folic acid antagonist.

13895. The method of item 13868 wherein the agent is a cytidine analogue.

13896. The method of item 13868 wherein the agent is a pyrimidine analogue.

13897. The method of item 13868 wherein the agent is a fluoropyrimidine analogue.

13898. The method of item 13868 wherein the agent is a purine analogue.

13899. The method of item 13868 wherein the agent is a nitrogen mustard or an analogue or derivative thereof.

13900. The method of item 13868 wherein the agent is a hydroxyurea.

13901. The method of item 13868 wherein the agent is a mytomicin or an analogue or derivative thereof.

13902. The method of item 13868 wherein the agent is an alkyl sulfonate.

13903. The method of item 13868 wherein the agent is a benzamide or an analogue or derivative thereof.

13904. The method of item 13868 wherein the agent is a nicotinamide or an analogue or derivative thereof.

13905. The method of item 13868 wherein the agent is a halogenated sugar or an analogue or derivative thereof.

13906. The method of item 13868 wherein the agent is a DNA alkylating agent.

13907. The method of item 13868 wherein the agent is an anti-microtubule agent.

13908. The method of item 13868 wherein the agent is a topoisomerase inhibitor.

13909. The method of item 13868 wherein the agent is a DNA cleaving agent.

13910. The method of item 13868 wherein the agent is an antimetabolite.

13911. The method of item 13868 wherein the agent inhibits adenosine deaminase.

13912. The method of item 13868 wherein the agent inhibits purine ring synthesis.

13913. The method of item 13868 wherein the agent is a nucleotide interconversion inhibitor.

13914. The method of item 13868 wherein the agent inhibits dihydrofolate reduction.

13915. The method of item 13868 wherein the agent blocks thymidine monophosphate.

13916. The method of item 13868 wherein the agent causes DNA damage.

13917. The method of item 13868 wherein the agent is a DNA intercalation agent.

13918. The method of item 13868 wherein the agent is a RNA synthesis inhibitor.

13919. The method of item 13868 wherein the agent is a pyrimidine synthesis inhibitor.

13920. The method of item 13868 wherein the agent inhibits ribonucleotide synthesis or function.

13921. The method of item 13868 wherein the agent inhibits thymidine monophosphate synthesis or function.

13922. The method of item 13868 wherein the agent inhibits DNA synthesis.

13923. The method of item 13868 wherein the agent causes DNA adduct formation.

13924. The method of item 13868 wherein the agent inhibits protein synthesis.

13925. The method of item 13868 wherein the agent inhibits microtubule function.

13926. The method of item 13868 wherein the agent is a cyclin dependent protein kinase inhibitor.

13927. The method of item 13868 wherein the agent is an epidermal growth factor kinase inhibitor.

13928. The method of item 13868 wherein the agent is an elastase inhibitor.

13929. The method of item 13868 wherein the agent is a factor Xa inhibitor.

13930. The method of item 13868 wherein the agent is a farnesyltransferase inhibitor.

13931. The method of item 13868 wherein the agent is a fibrinogen antagonist.

13932. The method of item 13868 wherein the agent is a guanylate cyclase stimulant.

0.13933. The method of item 13868 wherein the agent is a heat shock protein 90 antagonist.

13934. The method of item 13868 wherein the agent is a heat shock protein 90 antagonist, wherein the heat shock protein 90 antagonist is geldanamycin or an analogue or derivative thereof.

13935. The method of item 13868 wherein the agent is a guanylate cyclase stimulant.

13936. The method of item 13868 wherein the agent is a HMGCoA reductase inhibitor.

13937. The method of item 13868 wherein the agent is a HMGCoA reductase inhibitor, wherein the HMGCoA reductase inhibitor is simvastatin or an analogue or derivative thereof.

13938. The method of item 13868 wherein the agent is a hydroorotate dehydrogenase inhibitor.

13939. The method of item 13868 wherein the agent is an IKK2 inhibitor.

13940. The method of item 13868 wherein the agent is an IL-1 antagonist.

13941. The method of item 13868 wherein the agent is an ICE antagonist.

13942. The method of item 13868 wherein the agent is an IRAK antagonist.

13943. The method of item 13868 wherein the agent is an IL-4 agonist.

13944. The method of item 13868 wherein the agent is an immunomodulatory agent.

13945. The method of item 13868 wherein the agent is sirolimus or an analogue or derivative thereof.

13946. The method of item 13868 wherein the agent is not sirolimus.

13947. The method of item 13868 wherein the agent is everolimus or an analogue or derivative thereof.

13948. The method of item 13868 wherein the agent is tacrolimus or an analogue or derivative thereof.

13949. The method of item 13868 wherein the agent is not tacrolimus.

13950. The method of item 13868 wherein the agent is biolmus or an analogue or derivative thereof.

13951. The method of item 13868 wherein the agent is tresperimus or an analogue or derivative thereof.

13952. The method of item 13868 wherein the agent is auranofin or an analogue or derivative thereof.

13953. The method of item 13868 wherein the agent is 27-0-demethylrapamycin or an analogue or derivative thereof.

13954. The method of item 13868 wherein the agent is gusperimus or an analogue or derivative thereof.

13955. The method of item 13868 wherein the agent is pimecrolimus or an analogue or derivative thereof.

13956. The method of item 13868 wherein the agent is ABT-578 or an analogue or derivative thereof.

13957. The method of item 13868 wherein the agent is an inosine monophosphate dehydrogenase (IMPDH) inhibitor.

13958. The method of item 13868 wherein the agent is an IMPDH inhibitor, wherein the IMPDH inhibitor is mycophenolic acid or an analogue or derivative thereof.

13959. The method of item 13868 wherein the agent is an IMPDH inhibitor, wherein the IMPDH inhibitor is 1-alpha-25 dihydroxy vitamin D₃ or an analogue or derivative thereof.

13960. The method of item 13868 wherein the agent is a leukotriene inhibitor.

13961. The method of item 13868 wherein the agent is a MCP-1 antagonist.

13962. The method of item 13868 wherein the agent is a MMP inhibitor.

13963. The method of item 13868 wherein the agent is an NF kappa B inhibitor.

13964. The method of item 13868 wherein the agent is an NF kappa B inhibitor, wherein the NF kappa B inhibitor is Bay 11-7082.

13965. The method of item 13868 wherein the agent is an NO agonist.

13966. The method of item 13868 wherein the agent is a p38 MAP kinase inhibitor.

13967. The method of item 13868 wherein the agent is a p38 MAP kinase inhibitor, wherein the p38 MAP kinase inhibitor is SB 202190.

13968. The method of item 13868 wherein the agent is a phosphodiesterase inhibitor.

13969. The method of item 13868 wherein the agent is a TGF beta inhibitor.

13970. The method of item 13868 wherein the agent is a thromboxane A2 antagonist.

13971. The method of item 13868 wherein the agent is a TNFa antagonist.

13972. The method of item 13868 wherein the agent is a TACE inhibitor.

13973. The method of item 13868 wherein the agent is a tyrosine kinase inhibitor.

13974. The method of item 13868 wherein the agent is a vitronectin inhibitor.

13975. The method of item 13868 wherein the agent is a fibroblast growth factor inhibitor.

13976. The method of item 13868 wherein the agent is a protein kinase inhibitor.

13977. The method of item 13868 wherein the agent is a PDGF receptor kinase inhibitor.

13978. The method of item 13868 wherein the agent is an endothelial growth factor receptor kinase inhibitor.

13979. The method of item 13868 wherein the agent is a retinoic acid receptor antagonist.

13980. The method of item 13868 wherein the agent is a platelet derived growth factor receptor kinase inhibitor.

13981. The method of item 13868 wherein the agent is a fibronogin antagonist.

13982. The method of item 13868 wherein the agent is an antimycotic agent.

13983. The method of item 13868 wherein the agent is an antimycotic agent, wherein the antimycotic agent is sulconizole.

13984. The method of item 13868 wherein the agent is a bisphosphonate.

13985. The method of item 13868 wherein the agent is a phospholipase A1 inhibitor.

13986. The method of item 13868 wherein the agent is a histamine H1/H2/H3 receptor antagonist.

13987. The method of item 13868 wherein the agent is a macrolide antibiotic.

13988. The method of item 13868 wherein the agent is a GPIIb/IIIa receptor antagonist.

13989. The method of item 13868 wherein the agent is an endothelin receptor antagonist.

13990. The method of item 13868 wherein the agent is a peroxisome proliferator-activated receptor agonist.

13991. The method of item 13868 wherein the agent is an estrogen receptor agent.

13992. The method of item 13868 wherein the agent is a somastostatin analogue.

13993. The method of item 13868 wherein the agent is a neurokinin 1 antagonist.

13994. The method of item 13868 wherein the agent is a neurokinin 3 antagonist.

13995. The method of item 13868 wherein the agent is a VLA-4 antagonist.

13996. The method of item 13868 wherein the agent is an osteoclast inhibitor.

13997. The method of item 13868 wherein the agent is a DNA topoisomerase ATP hydrolyzing inhibitor.

13998. The method of item 13868 wherein the agent is an angiotensin I converting enzyme inhibitor.

13999. The method of item 13868 wherein the agent is an angiotensin II antagonist.

14000. The method of item 13868 wherein the agent is an enkephalinase inhibitor.

14001. The method of item 13868 wherein the agent is a peroxisome proliferator-activated receptor gamma agonist insulin sensitizer.

14002. The method of item 13868 wherein the agent is a protein kinase C inhibitor.

14003. The method of item 13868 wherein the agent is a ROCK (rho-associated kinase) inhibitor.

14004. The method of item 13868 wherein the agent is a CXCR3 inhibitor.

14005. The method of item 13868 wherein the agent is an Itk inhibitor.

14006. The method of item 13868 wherein the agent is a cytosolic phospholipase A₂-alpha inhibitor.

14007. The method of item 13868 wherein the agent is a PPAR agonist.

14008. The method of item 13868 wherein the agent is an immunosuppressant.

14009. The method of item 13868 wherein the agent is an Erb inhibitor.

14010. The method of item 13868 wherein the agent is an apoptosis agonist.

14011. The method of item 13868 wherein the agent is a lipocortin agonist.

14012. The method of item 13868 wherein the agent is a VCAM-1 antagonist.

14013. The method of item 13868 wherein the agent is a collagen antagonist.

14014. The method of item 13868 wherein the agent is an alpha 2 integrin antagonist.

14015. The method of item 13868 wherein the agent is a TNF alpha inhibitor.

14016. The method of item 13868 wherein the agent is a nitric oxide inhibitor.

14017. The method of item 13868 wherein the agent is a cathepsin inhibitor.

14018. The method of item 13868 wherein the agent is not an anti-inflammatory agent.

14019. The method of item 13868 wherein the agent is not a steroid.

14020. The method of item 13868 wherein the agent is not a glucocorticosteroid.

14021. The method of item 13868 wherein the agent is not dexamethasone.

14022. The method of item 13868 wherein the agent is not an anti-infective agent.

14023. The method of item 13868 wherein the agent is not an antibiotic.

14024. The method of item 13868 wherein the agent is not an anti-fungal agent.

14025. The method of item 13868, wherein the composition comprises a polymer.

14026. The method of item 13868, wherein the composition comprises a polymeric carrier.

14027. The method of item 13868 wherein the anti-scarring agent inhibits adhesion between the device and a host into which the device is implanted.

14028. The method of item 13868 wherein the device delivers the anti-scarring agent locally to tissue proximate to the device.

14029. The method of item 13868 wherein the device has a coating that comprises the anti-scarring agent.

14030. The method of item 13868, wherein the device has a coating that comprises the agent and is disposed on a surface of the implant.

14031. The method of item 13868, wherein the device has a coating that comprises the agent and directly contacts the implant.

14032. The method of item 13868, wherein the device has a coating that comprises the agent and indirectly contacts the implant.

14033. The method of item 13868, wherein the device has a coating that comprises the agent and partially covers the implant.

14034. The method of item 13868, wherein the device has a coating that comprises the agent and completely covers the implant.

14035. The method of item 13868, wherein the device has a uniform coating.

14036. The method of item 13868, wherein the device has a non-uniform coating.

14037. The method of item 13868, wherein the device has a discontinuous coating.

14038. The method of item 13868, wherein the device has a patterned coating.

14039. The method of item 13868, wherein the device has a coating with a thickness of 100 μm or less.

14040. The method of item 13868, wherein the device has a coating with a thickness of 10 μm or less.

14041. The method of item 13868, wherein the device has a coating, and the coating adheres to the surface of the implant upon deployment of the implant.

14042. The method of item 13868, wherein the device has a coating, and wherein the coating is stable at room temperature for a period of 1 year.

14043. The method of item 13868, wherein the device has a coating, and wherein the anti-scarring agent is present in the coating in an amount ranging between about 0.0001% to about 1% by weight.

14044. The method of item 13868, wherein the device has a coating, and wherein the anti-scarring agent is present in the coating in an amount ranging between about 1% to about 10% by weight.

14045. The method of item 13868, wherein the device has a coating, and wherein the anti-scarring agent is present in the coating in an amount ranging between about 10% to about 25% by weight.

14046. The method of item 13868, wherein the device has a coating, and wherein the anti-scarring agent is present in the coating in an amount ranging between about 25% to about 70% by weight.

14047. The method of item 13868, wherein the device has a coating, and wherein the coating further comprises a polymer.

14048. The method of item 13868, wherein the device has a first coating having a first composition and a second coating having a second composition.

14049. The method of item 13868, wherein the device has a first coating having a first composition and a second coating having a second composition, wherein the first composition and the second composition are different.

14050. The method of item 13868, wherein the composition comprises a polymer.

14051. The method of item 13868, wherein the composition comprises a polymeric carrier.

14052. The method of item 13868, wherein the composition comprises a polymeric carrier, and wherein the polymeric carrier comprises a copolymer.

14053. The method of item 13868, wherein the composition comprises a polymeric carrier, and wherein the polymeric carrier comprises a block copolymer.

14054. The method of item 13868, wherein the composition comprises a polymeric carrier, and wherein the polymeric carrier comprises a random copolymer.

14055. The method of item 13868, wherein the composition comprises a polymeric carrier, and wherein the polymeric carrier comprises a biodegradable polymer.

14056. The method of item 13868, wherein the composition comprises a polymeric carrier, and wherein the polymeric carrier comprises a non-biodegradable polymer.

14057. The method of item 13868, wherein the composition comprises a polymeric carrier, and wherein the polymeric carrier comprises a hydrophilic polymer.

14058. The method of item 13868, wherein the composition comprises a polymeric carrier, and wherein the polymeric carrier comprises a hydrophobic polymer.

14059. The method of item 13868, wherein the composition comprises a polymeric carrier, and wherein the polymeric carrier comprises a polymer having hydrophilic domains.

14060. The method of item 13868, wherein the composition comprises a polymeric carrier, and wherein the polymeric carrier comprises a polymer having hydrophobic domains.

14061. The method of item 13868, wherein the composition comprises a polymeric carrier, and wherein the polymeric carrier comprises a non-conductive polymer.

14062. The method of item 13868, wherein the composition comprises a polymeric carrier, and wherein the polymeric carrier comprises an elastomer.

14063. The method of item 13868, wherein the composition comprises a polymeric carrier, and wherein the polymeric carrier comprises a hydrogel.

14064. The method of item 13868, wherein the composition comprises a polymeric carrier, and wherein the polymeric carrier comprises a silicone polymer.

14065. The method of item 13868, wherein the composition comprises a polymeric carrier, and wherein the polymeric carrier comprises a hydrocarbon polymer.

14066. The method of item 13868, wherein the composition comprises a polymeric carrier, and wherein the polymeric carrier comprises a styrene-derived polymer.

14067. The method of item 13868, wherein the composition comprises a polymeric carrier, and wherein the polymeric carrier comprises a butadiene polymer.

14068. The method of item 13868, wherein the composition comprises a polymeric carrier, and wherein the polymeric carrier comprises a macromer.

14069. The method of item 13868, wherein the composition comprises a polymeric carrier, and wherein the polymeric carrier comprises a poly(ethylene glycol)polymer.

14070. The method of item 13868 wherein the composition comprises a polymeric carrier, and wherein the polymeric carrier comprises an amorphous polymer.

14071. The method of item 13868, wherein the device comprises a lubricious coating.

14072. The method of item 13868 wherein the anti-scarring agent is located within pores or holes of the device.

14073. The method of item 13868 wherein the anti-scarring agent is located within a channel, lumen, or divet of the device.

14074. The method of item 13868, wherein the device comprises a second pharmaceutically active agent.

14075. The method of item 13868 wherein the device comprises an anti-inflammatory agent.

14076. The method of item 13868 wherein the device comprises an agent that inhibits infection.

14077. The method of item 13868 wherein the device comprises an agent that inhibits infection, and wherein the agent is an anthracycline.

14078. The method of item 13868 wherein the device comprises an agent that inhibits infection, and wherein the agent is doxorubicin.

14079. The method of item 13868 wherein the device comprises an agent that inhibits infection, and wherein the agent is mitoxantrone.

14080. The method of item 13868 wherein the device comprises an agent that inhibits infection, and wherein the agent is a fluoropyrimidine.

14081. The method of item 13868 wherein the device comprises an agent that inhibits infection, and wherein the agent is 5-fluorouracil (5-FU).

14082. The method of item 13868 wherein the device comprises an agent that inhibits infection, and wherein the agent is a folic acid antagonist.

14083. The method of item 13868 wherein the device comprises an agent that inhibits infection, and wherein the agent is methotrexate.

14084. The method of item 13868 wherein the device comprises an agent that inhibits infection, and wherein the agent is a podophylotoxin.

14085. The method of item 13868 wherein the device comprises an agent that inhibits infection, and wherein the agent is etoposide.

14086. The method of item 13868 wherein the device comprises an agent that inhibits infection, and wherein the agent is a camptothecin.

14087. The method of item 13868 wherein the device comprises an agent that inhibits infection, and wherein the agent is a hydroxyurea.

14088. The method of item 13868 wherein the device comprises an agent that inhibits infection, and wherein the agent is a platinum complex.

14089. The method of item 13868 wherein the device comprises an agent that inhibits infection, and wherein the agent is cisplatin.

14090. The method of item 13868, further comprising an anti-thrombotic agent.

14091. The method of item 13868 wherein the device comprises a visualization agent.

14092. The method of item 13868 wherein the device comprises a visualization agent, wherein the visualization agent is a radiopaque material, and wherein the radiopaque material comprises a metal, a halogenated compound, or a barium containing compound.

14093. The method of item 13868 wherein the device comprises a visualization agent, wherein the visualization agent is a radiopaque material, and wherein the radiopaque material comprises barium, tantalum, or technetium.

14094. The method of item 13868 wherein the device comprises a visualization agent, and wherein the visualization agent is a MRI responsive material.

14095. The method of item 13868 wherein the device comprises a visualization agent, and wherein the visualization agent comprises a gadolinium chelate.

14096. The method of item 13868 wherein the device comprises a visualization agent, and wherein the visualization agent comprises iron, magnesium, manganese, copper, or chromium.

14097. The method of item 13868 wherein the device comprises a visualization agent, and wherein the visualization agent comprises an iron oxide compound.

14098. The method of item 13868 wherein the device comprises a visualization agent, and wherein the visualization agent comprises a dye, pigment, or colorant.

14099. The method of item 13868 wherein the device comprises an echogenic material.

14100. The method of item 13868 wherein the device comprises an echogenic material, and wherein the echogenic material is in the form of a coating.

14101. The method of item 13868 wherein the device is sterile.

14102. The method of item 13868 wherein the anti-scarring agent is released into tissue in the vicinity of the device after deployment of the device.

14103. The method of item 13868 wherein the anti-scarring agent is released into tissue in the vicinity of the device after deployment of the device, and wherein the tissue is connective tissue.

14104. The method of item 13868 wherein the anti-scarring agent is released into tissue in the vicinity of the device after deployment of the device, and wherein the tissue is muscle tissue.

14105. The method of item 13868 wherein the anti-scarring agent is released into tissue in the vicinity of the device after deployment of the device, and wherein the tissue is nerve tissue.

14106. The method of item 13868 wherein the anti-scarring agent is released into tissue in the vicinity of the device after deployment of the device, and wherein the tissue is epithelium tissue.

14107. The method of item 13868 wherein the anti-scarring agent is released in effective concentrations from the device over a period ranging from the time of deployment of the device to about 1 year.

14108. The method of item 13868 wherein the anti-scarring agent is released in effective concentrations from the device over a period ranging from about 1 month to 6 months.

14109. The method of item 13868 wherein the anti-scarring agent is released in effective concentrations from the device over a period ranging from about 1-90 days.

14110. The method of item 13868 wherein the anti-scarring agent is released in effective concentrations from the device at a constant rate.

14111. The method of item 13868 wherein the anti-scarring agent is released in effective concentrations from the device at an increasing rate.

14112. The method of item 13868 wherein the anti-scarring agent is released in effective concentrations from the device at a decreasing rate.

14113. The method of item 13868 wherein the anti-scarring agent is released in effective concentrations from the composition comprising the anti-scarring agent by diffusion over a period ranging from the time of deployment of the device to about 90 days.

14114. The method of item 13868 wherein the anti-scarring agent is released in effective concentrations from the composition comprising the anti-scarring agent by erosion of the composition over a period ranging from the time of deployment of the device to about 90 days.

14115. The method of item 13868 wherein the device comprises about 0.01 μg to about 10 μg of the anti-scarring agent.

14116. The method of item 13868 wherein the device comprises about 10 μg to about 10 mg of the anti-scarring agent.

14117. The method of item 13868 wherein the device comprises about 10 mg to about 250 mg of the anti-scarring agent.

14118. The method of item 13868 wherein the device comprises about 250 mg to about 1000 mg of the anti-scarring agent.

14119. The method of item 13868 wherein the device comprises about 1000 mg to about 2500 mg of the anti-scarring agent.

14120. The method of item 13868 wherein a surface of the device comprises less than 0.01 μg of the anti-scarring agent per mm² of device surface to which the anti-scarring agent is applied.

14121. The method of item 13868 wherein a surface of the device comprises about 0.01 μg to about 1 μg of the anti-scarring agent per mm² of device surface to which the anti-scarring agent is applied.

14122. The method of item 13868 wherein a surface of the device comprises about 1 μg to about 10 μg of the anti-scarring agent per mm² of device surface to which the anti-scarring agent is applied.

14123. The method of item 13868 wherein a surface of the device comprises about 10 μg to about 250 μg of the anti-scarring agent per mm² of device surface to which the anti-scarring agent is applied.

14124. The method of item 13868 wherein a surface of the device comprises about 250 μg to about 1000 μg of the anti-scarring agent of anti-scarring agent per mm² of device surface to which the anti-scarring agent is applied.

14125. The method of item 13868 wherein a surface of the device comprises about 1000 μg to about 2500 μg of the anti-scarring agent per mm² of device surface to which the anti-scarring agent is applied.

14126. The method of item 13868 wherein the combining is performed by direct affixing the agent or the composition to the implant.

14127. The method of item 13868 wherein the combining is performed by spraying the agent or the component onto the implant.

14128. The method of item 13868 wherein the combining is performed by electrospraying the agent or the composition onto the implant.

14129. The method of item 13868 wherein the combining is performed by dipping the implant into a solution comprising the agent or the composition.

14130. The method of item 13868 wherein the combining is performed by covalently attaching the agent or the composition to the implant.

14131. The method of item 13868 wherein the combining is performed by non-covalently attaching the agent or the composition to the implant.

14132. The method of item 13868 wherein the combining is performed by coating the implant with a substance that contains the agent or the composition.

14133. The method of item 13868 wherein the combining is performed by coating the implant with a substance that absorbs the agent.

14134. The method of item 13868 wherein the combining is performed by interweaving a thread composed of, or coated with, the agent or the composition.

14135. The method of item 13868 wherein the combining is performed by covering all the implant with a sleeve that contains the agent or the composition.

14136. The method of item 13868 wherein the combining is performed by covering a portion of the implant with a sleeve that contains the agent or the composition.

14137. The method of item 13868 wherein the combining is performed by covering all the implant with a cover that contains the agent or the composition.

14138. The method of item 13868 wherein the combining is performed by covering a portion of the implant with a cover that contains the agent or the composition.

14139. The method of item 13868 wherein the combining is performed by covering all the implant with an electrospun fabric that contains the agent or the composition.

14140. The method of item 13868 wherein the combining is performed by covering a portion of the implant with an electrospun fabric that contains the agent or the composition.

14141. The method of item 13868 wherein the combining is performed by covering all the implant with a mesh that contains the agent or the composition.

14142. The method of item 13868 wherein the combining is performed by covering a portion of the implant with a mesh that contains the agent or the composition.

14143. The method of item 13868 wherein the combining is performed by constructing all the implant with the agent or the composition.

14144. The method of item 13868 wherein the combining is performed by constructing a portion of the implant with the agent or the composition.

14145. The method of item 13868 wherein the combining is performed by impregnating the implant with the agent or the composition.

14146. The method of item 13868 wherein the combining is performed by constructing all of the implant from a degradable polymer that releases the agent.

14147. The method of item 13868 wherein the combining is performed by constructing a portion of the implant from a degradable polymer that releases the agent.

14148. The method of item 13868 wherein the combining is performed by dipping the implant into a solution that comprise the agent and an inert solvent for the implant.

14149. The method of item 13868 wherein the combining is performed by dipping the implant into a solution that comprises the agent and a solvent that will swill the implant.

14150. The method of item 13868 wherein the combining is performed by dipping the implant into a solution that comprises the agent and a solvent that will dissolve the implant.

14151. The method of item 13868 wherein the combining is performed by dipping the implant into a solution that comprises the agent, a polymer and an inert solvent for the implant.

14152. The method of item 13868 wherein the combining is performed by dipping the implant into a solution that comprises the agent, a polymer and a solvent that will swill the implant.

14153. The method of item 13868 wherein the combining is performed by dipping the implant into a solution that comprises the agent, a polymer and a solvent that will dissolve the implant.

14154. The method of item 13868 wherein the combining is performed by spraying the implant into a solution that comprises the agent and an inert solvent for the implant.

14155. The method of item 13868 wherein the combining is performed by spraying the implant into a solution that comprises the agent and a solvent that will swill the implant.

14156. The method of item 13868 wherein the combining is performed by spraying the implant into a solution that comprises the agent and a solvent that will dissolve the implant.

14157. The method of item 13868 wherein the combining is performed by spraying the implant into a solution that comprises the agent, a polymer and an inert solvent for the implant.

14158. The method of item 13868 wherein the combining is performed by spraying the implant into a solution that comprises the agent, a polymer and a solvent that will swill the implant.

14159. The method of item 13868 wherein the combining is performed by spraying the implant into a solution that comprises the agent, a polymer and a solvent that will dissolve the implant.

14160. The method of item 13868 wherein the implant is an episcleral drainage plate or tube.

14161. A method of making a medical device comprising: combining a penile implant and an anti-scarring agent or a composition comprising an anti-scarring agent, wherein the agent inhibits scarring between the device and a host into which the device is implanted.

14162. The method of item 14161 wherein the agent inhibits cell regeneration.

14163. The method of item 14161 wherein the agent inhibits angiogenesis.

14164. The method of item 14161 wherein the agent inhibits fibroblast migration.

14165. The method of item 14161 wherein the agent inhibits fibroblast proliferation.

14166. The method of item 14161 wherein the agent inhibits deposition of extracellular matrix.

14167. The method of item 14161 wherein the agent inhibits tissue remodeling.

14168. The method of item 14161 wherein the agent is an angiogenesis inhibitor.

14169. The method of item 14161 wherein the agent is a 5-lipoxygenase inhibitor or antagonist.

14170. The method of item 14161 wherein the agent is a chemokine receptor antagonist.

14171. The method of item 14161 wherein the agent is a cell cycle inhibitor.

14172. The method of item 14161 wherein the agent is a taxane.

14173. The method of item 14161 wherein the agent is an anti-microtubule agent.

14174. The method of item 14161 wherein the agent is paclitaxel.

14175. The method of item 14161 wherein the agent is not paclitaxel.

14176. The method of item 14161 wherein the agent is an analogue or derivative of paclitaxel.

14177. The method of item 14161 wherein the agent is a vinca alkaloid.

14178. The method of item 14161 wherein the agent is camptothecin or an analogue or derivative thereof.

14179. The method of item 14161 wherein the agent is a podophyllotoxin.

14180. The method of item 14161 wherein the agent is a podophyllotoxin, wherein the podophyllotoxin is etoposide or an analogue or derivative thereof.

14181. The method of item 14161 wherein the agent is an anthracycline.

14182. The method of item 14161 wherein the agent is an anthracycline, wherein the anthracycline is doxorubicin or an analogue or derivative thereof.

14183. The method of item 14161 wherein the agent is an anthracycline, wherein the anthracycline is mitoxantrone or an analogue or derivative thereof.

14184. The method of item 14161 wherein the agent is a platinum compound.

14185. The method of item 14161 wherein the agent is a nitrosourea.

14186. The method of item 14161 wherein the agent is a nitroimidazole.

14187. The method of item 14161 wherein the agent is a folic acid antagonist.

14188. The method of item 14161 wherein the agent is a cytidine analogue.

14189. The method of item 14161 wherein the agent is a pyrimidine analogue.

14190. The method of item 14161 wherein the agent is a fluoropyrimidine analogue.

14191. The method of item 14161 wherein the agent is a purine analogue.

14192. The method of item 14161 wherein the agent is a nitrogen mustard or an analogue or derivative thereof.

14193. The method of item 14161 wherein the agent is a hydroxyurea.

14194. The method of item 14161 wherein the agent is a mytomicin or an analogue or derivative thereof.

14195. The method of item 14161 wherein the agent is an alkyl sulfonate.

14196. The method of item 14161 wherein the agent is a benzamide or an analogue or derivative thereof.

14197. The method of item 14161 wherein the agent is a nicotinamide or an analogue or derivative thereof.

14198. The method of item 14161 wherein the agent is a halogenated sugar or an analogue or derivative thereof.

14199. The method of item 14161 wherein the agent is a DNA alkylating agent.

14200. The method of item 14161 wherein the agent is an anti-microtubule agent.

14201. The method of item 14161 wherein the agent is a topoisomerase inhibitor.

14202. The method of item 14161 wherein the agent is a DNA cleaving agent.

14203. The method of item 14161 wherein the agent is an antimetabolite.

14204. The method of item 14161 wherein the agent inhibits adenosine deaminase.

14205. The method of item 14161 wherein the agent inhibits purine ring synthesis.

14206. The method of item 14161 wherein the agent is a nucleotide interconversion inhibitor.

14207. The method of item 14161 wherein the agent inhibits dihydrofolate reduction.

14208. The method of item 14161 wherein the agent blocks thymidine monophosphate.

14209. The method of item 14161 wherein the agent causes DNA damage.

14210. The method of item 14161 wherein the agent is a DNA intercalation agent.

14211. The method of item 14161 wherein the agent is a RNA synthesis inhibitor.

14212. The method of item 14161 wherein the agent is a pyrimidine synthesis inhibitor.

14213. The method of item 14161 wherein the agent inhibits ribonucleotide synthesis or function.

14214. The method of item 14161 wherein the agent inhibits thymidine monophosphate synthesis or function.

14215. The method of item 14161 wherein the agent inhibits DNA synthesis.

14216. The method of item 14161 wherein the agent causes DNA adduct formation.

14217. The method of item 14161 wherein the agent inhibits protein synthesis.

14218. The method of item 14161 wherein the agent inhibits microtubule function.

14219. The method of item 14161 wherein the agent is a cyclin dependent protein kinase inhibitor.

14220. The method of item 14161 wherein the agent is an epidermal growth factor kinase inhibitor.

14221. The method of item 14161 wherein the agent is an elastase inhibitor.

14222. The method of item 14161 wherein the agent is a factor Xa inhibitor.

14223. The method of item 14161 wherein the agent is a farnesyltransferase inhibitor.

14224. The method of item 14161 wherein the agent is a fibrinogen antagonist.

14225. The method of item 14161 wherein the agent is a guanylate cyclase stimulant.

14226. The method of item 14161 wherein the agent is a heat shock protein 90 antagonist.

14227. The method of item 14161 wherein the agent is a heat shock protein 90 antagonist, wherein the heat shock protein 90 antagonist is geldanamycin or an analogue or derivative thereof.

14228. The method of item 14161 wherein the agent is a guanylate cyclase stimulant.

14229. The method of item 14161 wherein the agent is a HMGCoA reductase inhibitor.

14230. The method of item 14161 wherein the agent is a HMGCoA reductase inhibitor, wherein the HMGCoA reductase inhibitor is simvastatin or an analogue or derivative thereof.

14231. The method of item 14161 wherein the agent is a hydroorotate dehydrogenase inhibitor.

14232. The method of item 14161 wherein the agent is an IKK2 inhibitor.

14233. The method of item 14161 wherein the agent is an IL-1 antagonist.

14234. The method of item 14161 wherein the agent is an ICE antagonist.

14235. The method of item 14161 wherein the agent is an IRAK antagonist.

14236. The method of item 14161 wherein the agent is an IL-4 agonist.

14237. The method of item 14161 wherein the agent is an immunomodulatory agent.

14238. The method of item 14161 wherein the agent is sirolimus or an analogue or derivative thereof.

14239. The method of item 14161 wherein the agent is not sirolimus.

14240. The method of item 14161 wherein the agent is everolimus or an analogue or derivative thereof.

14241. The method of item 14161 wherein the agent is tacrolimus or an analogue or derivative thereof.

14242. The method of item 14161 wherein the agent is not tacrolimus.

14243. The method of item 14161 wherein the agent is biolmus or an analogue or derivative thereof.

14244. The method of item 14161 wherein the agent is tresperimus or an analogue or derivative thereof.

14245. The method of item 14161 wherein the agent is auranofin or an analogue or derivative thereof.

14246. The method of item 14161 wherein the agent is 27-0-demethylrapamycin or an analogue or derivative thereof.

14247. The method of item 14161 wherein the agent is gusperimus or an analogue or derivative thereof.

14248. The method of item 14161 wherein the agent is pimecrolimus or an analogue or derivative thereof.

14249. The method of item 14161 wherein the agent is ABT-578 or an analogue or derivative thereof.

14250. The method of item 14161 wherein the agent is an inosine monophosphate dehydrogenase (IMPDH) inhibitor.

14251. The method of item 14161 wherein the agent is an IMPDH inhibitor, wherein the IMPDH inhibitor is mycophenolic acid or an analogue or derivative thereof.

14252. The method of item 14161 wherein the agent is an IMPDH inhibitor, wherein the IMPDH inhibitor is 1-alpha-25 dihydroxy vitamin D₃ or an analogue or derivative thereof.

14253. The method of item 14161 wherein the agent is a leukotriene inhibitor.

14254. The method of item 14161 wherein the agent is a MCP-1 antagonist.

14255. The method of item 14161 wherein the agent is a MMP inhibitor.

14256. The method of item 14161 wherein the agent is an NF kappa B inhibitor.

14257. The method of item 14161 wherein the agent is an NF kappa B inhibitor, wherein the NF kappa B inhibitor is Bay 11-7082.

14258. The method of item 14161 wherein the agent is an NO agonist.

14259. The method of item 14161 wherein the agent is a p38 MAP kinase inhibitor.

14260. The method of item 14161 wherein the agent is a p38 MAP kinase inhibitor, wherein the p38 MAP kinase inhibitor is SB 202190.

14261. The method of item 14161 wherein the agent is a phosphodiesterase inhibitor.

14262. The method of item 14161 wherein the agent is a TGF beta inhibitor.

14263. The method of item 14161 wherein the agent is a thromboxane A2 antagonist.

14264. The method of item 14161 wherein the agent is a TNFa antagonist.

14265. The method of item 14161 wherein the agent is a TACE inhibitor.

14266. The method of item 14161 wherein the agent is a tyrosine kinase inhibitor.

14267. The method of item 14161 wherein the agent is a vitronectin inhibitor.

14268. The method of item 14161 wherein the agent is a fibroblast growth factor inhibitor.

14269. The method of item 14161 wherein the agent is a protein kinase inhibitor.

14270. The method of item 14161 wherein the agent is a PDGF receptor kinase inhibitor.

14271. The method of item 14161 wherein the agent is an endothelial growth factor receptor kinase inhibitor.

14272. The method of item 14161 wherein the agent is a retinoic acid receptor antagonist.

14273. The method of item 14161 wherein the agent is a platelet derived growth factor receptor kinase inhibitor.

14274. The method of item 14161 wherein the agent is a fibronogin antagonist.

14275. The method of item 14161 wherein the agent is an antimycotic agent.

14276. The method of item 14161 wherein the agent is an antimycotic agent, wherein the antimycotic agent is sulconizole.

14277. The method of item 14161 wherein the agent is a bisphosphonate.

14278. The method of item 14161 wherein the agent is a phospholipase A1 inhibitor.

14279. The method of item 14161 wherein the agent is a histamine H1/H2/H3 receptor antagonist.

14280. The method of item 14161 wherein the agent is a macrolide antibiotic.

14281. The method of item 14161 wherein the agent is a GPIIb/IIIa receptor antagonist.

14282. The method of item 14161 wherein the agent is an endothelin receptor antagonist.

14283. The method of item 14161 wherein the agent is a peroxisome proliferator-activated receptor agonist.

14284. The method of item 14161 wherein the agent is an estrogen receptor agent.

14285. The method of item 14161 wherein the agent is a somastostatin analogue.

14286. The method of item 14161 wherein the agent is a neurokinin 1 antagonist.

14287. The method of item 14161 wherein the agent is a neurokinin 3 antagonist.

14288. The method of item 14161 wherein the agent is a VLA-4 antagonist.

14289. The method of item 14161 wherein the agent is an osteoclast inhibitor.

14290. The method of item 14161 wherein the agent is a DNA topoisomerase ATP hydrolyzing inhibitor.

14291. The method of item 14161 wherein the agent is an angiotensin I converting enzyme inhibitor.

14292. The method of item 14161 wherein the agent is an angiotensin II antagonist.

14293. The method of item 14161 wherein the agent is an enkephalinase inhibitor.

14294. The method of item 14161 wherein the agent is a peroxisome proliferator-activated receptor gamma agonist insulin sensitizer.

14295. The method of item 14161 wherein the agent is a protein kinase C inhibitor.

14296. The method of item 14161 wherein the agent is a ROCK (rho-associated kinase) inhibitor.

14297. The method of item 14161 wherein the agent is a CXCR3 inhibitor.

14298. The method of item 14161 wherein the agent is an Itk inhibitor.

14299. The method of item 14161 wherein the agent is a cytosolic phospholipase A₂-alpha inhibitor.

14300. The method of item 14161 wherein the agent is a PPAR agonist.

14301. The method of item 14161 wherein the agent is an immunosuppressant.

14302. The method of item 14161 wherein the agent is an Erb inhibitor.

14303. The method of item 14161 wherein the agent is an apoptosis agonist.

14304. The method of item 14161 wherein the agent is a lipocortin agonist.

14305. The method of item 14161 wherein the agent is a VCAM-1 antagonist.

14306. The method of item 14161 wherein the agent is a collagen antagonist.

14307. The method of item 14161 wherein the agent is an alpha 2 integrin antagonist.

14308. The method of item 14161 wherein the agent is a TNF alpha inhibitor.

14309. The method of item 14161 wherein the agent is a nitric oxide inhibitor.

14310. The method of item 14161 wherein the agent is a cathepsin inhibitor.

14311. The method of item 14161 wherein the agent is not an anti-inflammatory agent.

14312. The method of item 14161 wherein the agent is not a steroid.

14313. The method of item 14161 wherein the agent is not a glucocorticosteroid.

14314. The method of item 14161 wherein the agent is not dexamethasone.

14315. The method of item 14161 wherein the agent is not an anti-infective agent.

14316. The method of item 14161 wherein the agent is not an antibiotic.

14317. The method of item 14161 wherein the agent is not an anti-fungal agent.

14318. The method of item 14161, wherein the composition comprises a polymer.

14319. The method of item 14161, wherein the composition comprises a polymeric carrier.

14320. The method of item 14161 wherein the anti-scarring agent inhibits adhesion between the device and a host into which the device is implanted.

14321. The method of item 14161 wherein the device delivers the anti-scarring agent locally to tissue proximate to the device.

14322. The method of item 14161 wherein the device has a coating that comprises the anti-scarring agent.

14323. The method of item 14161, wherein the device has a coating that comprises the agent and is disposed on a surface of the implant.

14324. The method of item 14161, wherein the device has a coating that comprises the agent and directly contacts the implant.

14325. The method of item 14161, wherein the device has a coating that comprises the agent and indirectly contacts the implant.

14326. The method of item 14161, wherein the device has a coating that comprises the agent and partially covers the implant.

14327. The method of item 14161, wherein the device has a coating that comprises the agent and completely covers the implant.

14328. The method of item 14161, wherein the device has a uniform coating.

14329. The method of item 14161, wherein the device has a non-uniform coating.

14330. The method of item 14161, wherein the device has a discontinuous coating.

14331. The method of item 14161, wherein the device has a patterned coating.

14332. The method of item 14161, wherein the device has a coating with a thickness of 100 μm or less.

14333. The method of item 14161, wherein the device has a coating with a thickness of 10 μm or less.

14334. The method of item 14161, wherein the device has a coating, and the coating adheres to the surface of the implant upon deployment of the implant.

14335. The method of item 14161, wherein the device has a coating, and wherein the coating is stable at room temperature for a period of 1 year.

14336. The method of item 14161, wherein the device has a coating, and wherein the anti-scarring agent is present in the coating in an amount ranging between about 0.0001% to about 1% by weight.

14337. The method of item 14161, wherein the device has a coating, and wherein the anti-scarring agent is present in the coating in an amount ranging between about 1% to about 10% by weight.

14338. The method of item 14161, wherein the device has a coating, and wherein the anti-scarring agent is present in the coating in an amount ranging between about 10% to about 25% by weight.

14339. The method of item 14161, wherein the device has a coating, and wherein the anti-scarring agent is present in the coating in an amount ranging between about 25% to about 70% by weight.

14340. The method of item 14161, wherein the device has a coating, and wherein the coating further comprises a polymer.

14341. The method of item 14161, wherein the device has a first coating having a first composition and a second coating having a second composition.

14342. The method of item 14161, wherein the device has a first coating having a first composition and a second coating having a second composition, wherein the first composition and the second composition are different.

14343. The method of item 14161, wherein the composition comprises a polymer.

14344. The method of item 14161, wherein the composition comprises a polymeric carrier.

14345. The method of item 14161, wherein the composition comprises a polymeric carrier, and wherein the polymeric carrier comprises a copolymer.

14346. The method of item 14161, wherein the composition comprises a polymeric carrier, and wherein the polymeric carrier comprises a block copolymer.

14347. The method of item 14161, wherein the composition comprises a polymeric carrier, and wherein the polymeric carrier comprises a random copolymer.

14348. The method of item 14161, wherein the composition comprises a polymeric carrier, and wherein the polymeric carrier comprises a biodegradable polymer.

14349. The method of item 14161, wherein the composition comprises a polymeric carrier, and wherein the polymeric carrier comprises a non-biodegradable polymer.

14350. The method of item 14161, wherein the composition comprises a polymeric carrier, and wherein the polymeric carrier comprises a hydrophilic polymer.

14351. The method of item 14161, wherein the composition comprises a polymeric carrier, and wherein the polymeric carrier comprises a hydrophobic polymer.

14352. The method of item 14161, wherein the composition comprises a polymeric carrier, and wherein the polymeric carrier comprises a polymer having hydrophilic domains.

14353. The method of item 14161, wherein the composition comprises a polymeric carrier, and wherein the polymeric carrier comprises a polymer having hydrophobic domains.

14354. The method of item 14161, wherein the composition comprises a polymeric carrier, and wherein the polymeric carrier comprises a non-conductive polymer.

14355. The method of item 14161, wherein the composition comprises a polymeric carrier, and wherein the polymeric carrier comprises an elastomer.

14356. The method of item 14161, wherein the composition comprises a polymeric carrier, and wherein the polymeric carrier comprises a hydrogel.

14357. The method of item 14161, wherein the composition comprises a polymeric carrier, and wherein the polymeric carrier comprises a silicone polymer.

14358. The method of item 14161, wherein the composition comprises a polymeric carrier, and wherein the polymeric carrier comprises a hydrocarbon polymer.

14359. The method of item 14161, wherein the composition comprises a polymeric carrier, and wherein the polymeric carrier comprises a styrene-derived polymer.

14360. The method of item 14161, wherein the composition comprises a polymeric carrier, and wherein the polymeric carrier comprises a butadiene polymer.

14361. The method of item 14161, wherein the composition comprises a polymeric carrier, and wherein the polymeric carrier comprises a macromer.

14362. The method of item 14161, wherein the composition comprises a polymeric carrier, and wherein the polymeric carrier comprises a poly(ethylene glycol)polymer.

14363. The method of item 14161 wherein the composition comprises a polymeric carrier, and wherein the polymeric carrier comprises an amorphous polymer.

14364. The method of item 14161, wherein the device comprises a lubricious coating.

14365. The method of item 14161 wherein the anti-scarring agent is located within pores or holes of the device.

14366. The method of item 14161 wherein the anti-scarring agent is located within a channel, lumen, or divet of the device.

14367. The method of item 14161, wherein the device comprises a second pharmaceutically active agent.

14368. The method of item 14161 wherein the device comprises an anti-inflammatory agent.

14369. The method of item 14161 wherein the device comprises an agent that inhibits infection.

14370. The method of item 14161 wherein the device comprises an agent that inhibits infection, and wherein the agent is an anthracycline.

14371. The method of item 14161 wherein the device comprises an agent that inhibits infection, and wherein the agent is doxorubicin.

14372. The method of item 14161 wherein the device comprises an agent that inhibits infection, and wherein the agent is mitoxantrone.

14373. The method of item 14161 wherein the device comprises an agent that inhibits infection, and wherein the agent is a fluoropyrimidine.

14374. The method of item 14161 wherein the device comprises an agent that inhibits infection, and wherein the agent is 5-fluorouracil (5-FU).

14375. The method of item 14161 wherein the device comprises an agent that inhibits infection, and wherein the agent is a folic acid antagonist.

14376. The method of item 14161 wherein the device comprises an agent that inhibits infection, and wherein the agent is methotrexate.

14377. The method of item 14161 wherein the device comprises an agent that inhibits infection, and wherein the agent is a podophylotoxin.

14378. The method of item 14161 wherein the device comprises an agent that inhibits infection, and wherein the agent is etoposide.

14379. The method of item 14161 wherein the device comprises an agent that inhibits infection, and wherein the agent is a camptothecin.

14380. The method of item 14161 wherein the device comprises an agent that inhibits infection, and wherein the agent is a hydroxyurea.

14381. The method of item 14161 wherein the device comprises an agent that inhibits infection, and wherein the agent is a platinum complex.

14382. The method of item 14161 wherein the device comprises an agent that inhibits infection, and wherein the agent is cisplatin.

14383. The method of item 14161, further comprising an anti-thrombotic agent.

14384. The method of item 14161 wherein the device comprises a visualization agent.

14385. The method of item 14161 wherein the device comprises a visualization agent, wherein the visualization agent is a radiopaque material, and wherein the radiopaque material comprises a metal, a halogenated compound, or a barium containing compound.

14386. The method of item 14161 wherein the device comprises a visualization agent, wherein the visualization agent is a radiopaque material, and wherein the radiopaque material comprises barium, tantalum, or technetium.

14387. The method of item 14161 wherein the device comprises a visualization agent, and wherein the visualization agent is a MRI responsive material.

14388. The method of item 14161 wherein the device comprises a visualization agent, and wherein the visualization agent comprises a gadolinium chelate.

14389. The method of item 14161 wherein the device comprises a visualization agent, and wherein the visualization agent comprises iron, magnesium, manganese, copper, or chromium.

14390. The method of item 14161 wherein the device comprises a visualization agent, and wherein the visualization agent comprises an iron oxide compound.

14391. The method of item 14161 wherein the device comprises a visualization agent, and wherein the visualization agent comprises a dye, pigment, or colorant.

14392. The method of item 14161 wherein the device comprises an echogenic material.

14393. The method of item 14161 wherein the device comprises an echogenic material, and wherein the echogenic material is in the form of a coating.

14394. The method of item 14161 wherein the device is sterile.

14395. The method of item 14161 wherein the anti-scarring agent is released into tissue in the vicinity of the device after deployment of the device.

14396. The method of item 14161 wherein the anti-scarring agent is released into tissue in the vicinity of the device after deployment of the device, and wherein the tissue is connective tissue.

14397. The method of item 14161 wherein the anti-scarring agent is released into tissue in the vicinity of the device after deployment of the device, and wherein the tissue is muscle tissue.

14398. The method of item 14161 wherein the anti-scarring agent is released into tissue in the vicinity of the device after deployment of the device, and wherein the tissue is nerve tissue.

14399. The method of item 14161 wherein the anti-scarring agent is released into tissue in the vicinity of the device after deployment of the device, and wherein the tissue is epithelium tissue.

14400. The method of item 14161 wherein the anti-scarring agent is released in effective concentrations from the device over a period ranging from the time of deployment of the device to about 1 year.

14401. The method of item 14161 wherein the anti-scarring agent is released in effective concentrations from the device over a period ranging from about 1 month to 6 months.

14402. The method of item 14161 wherein the anti-scarring agent is released in effective concentrations from the device over a period ranging from about 1-90 days.

14403. The method of item 14161 wherein the anti-scarring agent is released in effective concentrations from the device at a constant rate.

14404. The method of item 14161 wherein the anti-scarring agent is released in effective concentrations from the device at an increasing rate.

14405. The method of item 14161 wherein the anti-scarring agent is released in effective concentrations from the device at a decreasing rate.

14406. The method of item 14161 wherein the anti-scarring agent is released in effective concentrations from the composition comprising the anti-scarring agent by diffusion over a period ranging from the time of deployment of the device to about 90 days.

14407. The method of item 14161 wherein the anti-scarring agent is released in effective concentrations from the composition comprising the anti-scarring agent by erosion of the composition over a period ranging from the time of deployment of the device to about 90 days.

14408. The method of item 14161 wherein the device comprises about 0.01 μg to about 10 μg of the anti-scarring agent.

14409. The method of item 14161 wherein the device comprises about 10 μg to about 10 mg of the anti-scarring agent.

14410. The method of item 14161 wherein the device comprises about 10 mg to about 250 mg of the anti-scarring agent.

14411. The method of item 14161 wherein the device comprises about 250 mg to about 1000 mg of the anti-scarring agent.

14412. The method of item 14161 wherein the device comprises about 1000 mg to about 2500 mg of the anti-scarring agent.

14413. The method of item 14161 wherein a surface of the device comprises less than 0.01 μg of the anti-scarring agent per mm² of device surface to which the anti-scarring agent is applied.

14414. The method of item 14161 wherein a surface of the device comprises about 0.01 μg to about 1 μg of the anti-scarring agent per mm² of device surface to which the anti-scarring agent is applied.

14415. The method of item 14161 wherein a surface of the device comprises about 1 μg to about 10 μg of the anti-scarring agent per mm² of device surface to which the anti-scarring agent is applied.

14416. The method of item 14161 wherein a surface of the device comprises about 10 μg to about 250 μg of the anti-scarring agent per mm² of device surface to which the anti-scarring agent is applied.

14417. The method of item 14161 wherein a surface of the device comprises about 250 μg to about 1000 μg of the anti-scarring agent of anti-scarring agent per mm² of device surface to which the anti-scarring agent is applied.

14418. The method of item 14161 wherein a surface of the device comprises about 1000 μg to about 2500 μg of the anti-scarring agent per mm² of device surface to which the anti-scarring agent is applied.

14419. The method of item 14161 wherein the combining is performed by direct affixing the agent or the composition to the implant.

14420. The method of item 14161 wherein the combining is performed by spraying the agent or the component onto the implant.

14421. The method of item 14161 wherein the combining is performed by electrospraying the agent or the composition onto the implant.

14422. The method of item 14161 wherein the combining is performed by dipping the implant into a solution comprising the agent or the composition.

14423. The method of item 14161 wherein the combining is performed by covalently attaching the agent or the composition to the implant.

14424. The method of item 14161 wherein the combining is performed by non-covalently attaching the agent or the composition to the implant.

14425. The method of item 14161 wherein the combining is performed by coating the implant with a substance that contains the agent or the composition.

14426. The method of item 14161 wherein the combining is performed by coating the implant with a substance that absorbs the agent.

14427. The method of item 14161 wherein the combining is performed by interweaving a thread composed of, or coated with, the agent or the composition.

14428. The method of item 14161 wherein the combining is performed by covering all the implant with a sleeve that contains the agent or the composition.

14429. The method of item 14161 wherein the combining is performed by covering a portion of the implant with a sleeve that contains the agent or the composition.

14430. The method of item 14161 wherein the combining is performed by covering all the implant with a cover that contains the agent or the composition.

14431. The method of item 14161 wherein the combining is performed by covering a portion of the implant with a cover that contains the agent or the composition.

14432. The method of item 14161 wherein the combining is performed by covering all the implant with an electrospun fabric that contains the agent or the composition.

14433. The method of item 14161 wherein the combining is performed by covering a portion of the implant with an electrospun fabric that contains the agent or the composition.

14434. The method of item 14161 wherein the combining is performed by covering all the implant with a mesh that contains the agent or the composition.

14435. The method of item 14161 wherein the combining is performed by covering a portion of the implant with a mesh that contains the agent or the composition.

14436. The method of item 14161 wherein the combining is performed by constructing all the implant with the agent or the composition.

14437. The method of item 14161 wherein the combining is performed by constructing a portion of the implant with the agent or the composition.

14438. The method of item 14161 wherein the combining is performed by impregnating the implant with the agent or the composition.

14439. The method of item 14161 wherein the combining is performed by constructing all of the implant from a degradable polymer that releases the agent.

14440. The method of item 14161 wherein the combining is performed by constructing a portion of the implant from a degradable polymer that releases the agent.

14441. The method of item 14161 wherein the combining is performed by dipping the implant into a solution that comprise the agent and an inert solvent for the implant.

14442. The method of item 14161 wherein the combining is performed by dipping the implant into a solution that comprises the agent and a solvent that will swill the implant.

14443. The method of item 14161 wherein the combining is performed by dipping the implant into a solution that comprises the agent and a solvent that will dissolve the implant.

14444. The method of item 14161 wherein the combining is performed by dipping the implant into a solution that comprises the agent, a polymer and an inert solvent for the implant.

14445. The method of item 14161 wherein the combining is performed by dipping the implant into a solution that comprises the agent, a polymer and a solvent that will swill the implant.

14446. The method of item 14161 wherein the combining is performed by dipping the implant into a solution that comprises the agent, a polymer and a solvent that will dissolve the implant.

14447. The method of item 14161 wherein the combining is performed by spraying the implant into a solution that comprises the agent and an inert solvent for the implant.

14448. The method of item 14161 wherein the combining is performed by spraying the implant into a solution that comprises the agent and a solvent that will swill the implant.

14449. The method of item 14161 wherein the combining is performed by spraying the implant into a solution that comprises the agent and a solvent that will dissolve the implant.

14450. The method of item 14161 wherein the combining is performed by spraying the implant into a solution that comprises the agent, a polymer and an inert solvent for the implant.

14451. The method of item 14161 wherein the combining is performed by spraying the implant into a solution that comprises the agent, a polymer and a solvent that will swill the implant.

14452. The method of item 14161 wherein the combining is performed by spraying the implant into a solution that comprises the agent, a polymer and a solvent that will dissolve the implant.

14453. The method of item 14161 wherein the implant is a flexible rod or coil.

14454. The method of item 14161 wherein the implant comprise an inflatable tube or a pump.

14455. The method of item 14161 wherein the implant comprises a pressure chamber.

14456. A method of making a medical device comprising: combining an endotracheal tube (i.e., an implant) and an anti-scarring agent or a composition comprising an anti-scarring agent, wherein the agent inhibits scarring between the device and a host into which the device is implanted.

14457. The method of item 14456 wherein the agent inhibits cell regeneration.

14458. The method of item 14456 wherein the agent inhibits angiogenesis.

14459. The method of item 14456 wherein the agent inhibits fibroblast migration.

14460. The method of item 14456 wherein the agent inhibits fibroblast proliferation.

14461. The method of item 14456 wherein the agent inhibits deposition of extracellular matrix.

14462. The method of item 14456 wherein the agent inhibits tissue remodeling.

14463. The method of item 14456 wherein the agent is an angiogenesis inhibitor.

14464. The method of item 14456 wherein the agent is a 5-lipoxygenase inhibitor or antagonist.

14465. The method of item 14456 wherein the agent is a chemokine receptor antagonist.

14466. The method of item 14456 wherein the agent is a cell cycle inhibitor.

14467. The method of item 14456 wherein the agent is a taxane.

14468. The method of item 14456 wherein the agent is an anti-microtubule agent.

14469. The method of item 14456 wherein the agent is paclitaxel.

14470. The method of item 14456 wherein the agent is not paclitaxel.

14471. The method of item 14456 wherein the agent is an analogue or derivative of paclitaxel.

14472. The method of item 14456 wherein the agent is a vinca alkaloid.

14473. The method of item 14456 wherein the agent is camptothecin or an analogue or derivative thereof.

14474. The method of item 14456 wherein the agent is a podophyllotoxin.

14475. The method of item 14456 wherein the agent is a podophyllotoxin, wherein the podophyllotoxin is etoposide or an analogue or derivative thereof.

14476. The method of item 14456 wherein the agent is an anthracycline.

14477. The method of item 14456 wherein the agent is an anthracycline, wherein the anthracycline is doxorubicin or an analogue or derivative thereof.

14478. The method of item 14456 wherein the agent is an anthracycline, wherein the anthracycline is mitoxantrone or an analogue or derivative thereof.

14479. The method of item 14456 wherein the agent is a platinum compound.

14480. The method of item 14456 wherein the agent is a nitrosourea.

14481. The method of item 14456 wherein the agent is a nitroimidazole.

14482. The method of item 14456 wherein the agent is a folic acid antagonist.

14483. The method of item 14456 wherein the agent is a cytidine analogue.

14484. The method of item 14456 wherein the agent is a pyrimidine analogue.

14485. The method of item 14456 wherein the agent is a fluoropyrimidine analogue.

14486. The method of item 14456 wherein the agent is a purine analogue.

14487. The method of item 14456 wherein the agent is a nitrogen mustard or an analogue or derivative thereof.

14488. The method of item 14456 wherein the agent is a hydroxyurea.

14489. The method of item 14456 wherein the agent is a mytomicin or an analogue or derivative thereof.

14490. The method of item 14456 wherein the agent is an alkyl sulfonate.

14491. The method of item 14456 wherein the agent is a benzamide or an analogue or derivative thereof.

14492. The method of item 14456 wherein the agent is a nicotinamide or an analogue or derivative thereof.

14493. The method of item 14456 wherein the agent is a halogenated sugar or an analogue or derivative thereof.

14494. The method of item 14456 wherein the agent is a DNA alkylating agent.

14495. The method of item 14456 wherein the agent is an anti-microtubule agent.

14496. The method of item 14456 wherein the agent is a topoisomerase inhibitor.

14497. The method of item 14456 wherein the agent is a DNA cleaving agent.

14498. The method of item 14456 wherein the agent is an antimetabolite.

14499. The method of item 14456 wherein the agent inhibits adenosine deaminase.

14500. The method of item 14456 wherein the agent inhibits purine ring synthesis.

14501. The method of item 14456 wherein the agent is a nucleotide interconversion inhibitor.

14502. The method of item 14456 wherein the agent inhibits dihydrofolate reduction.

14503. The method of item 14456 wherein the agent blocks thymidine monophosphate.

14504. The method of item 14456 wherein the agent causes DNA damage.

14505. The method of item 14456 wherein the agent is a DNA intercalation agent.

14506. The method of item 14456 wherein the agent is a RNA synthesis inhibitor.

14507. The method of item 14456 wherein the agent is a pyrimidine synthesis inhibitor.

14508. The method of item 14456 wherein the agent inhibits ribonucleotide synthesis or function.

14509. The method of item 14456 wherein the agent inhibits thymidine monophosphate synthesis or function.

14510. The method of item 14456 wherein the agent inhibits DNA synthesis.

14511. The method of item 14456 wherein the agent causes DNA adduct formation.

14512. The method of item 14456 wherein the agent inhibits protein synthesis.

14513. The method of item 14456 wherein the agent inhibits microtubule function.

14514. The method of item 14456 wherein the agent is a cyclin dependent protein kinase inhibitor.

14515. The method of item 14456 wherein the agent is an epidermal growth factor kinase inhibitor.

14516. The method of item 14456 wherein the agent is an elastase inhibitor.

14517. The method of item 14456 wherein the agent is a factor Xa inhibitor.

14518. The method of item 14456 wherein the agent is a farnesyltransferase inhibitor.

14519. The method of item 14456 wherein the agent is a fibrinogen antagonist.

14520. The method of item 14456 wherein the agent is a guanylate cyclase stimulant.

14521. The method of item 14456 wherein the agent is a heat shock protein 90 antagonist.

14522. The method of item 14456 wherein the agent is a heat shock protein 90 antagonist, wherein the heat shock protein 90 antagonist is geldanamycin or an analogue or derivative thereof.

14523. The method of item 14456 wherein the agent is a guanylate cyclase stimulant.

14524. The method of item 14456 wherein the agent is a HMGCoA reductase inhibitor.

14525. The method of item 14456 wherein the agent is a HMGCoA reductase inhibitor, wherein the HMGCoA reductase inhibitor is simvastatin or an analogue or derivative thereof.

14526. The method of item 14456 wherein the agent is a hydroorotate dehydrogenase inhibitor.

14527. The method of item 14456 wherein the agent is an IKK2 inhibitor.

14528. The method of item 14456 wherein the agent is an IL-1 antagonist.

14529. The method of item 14456 wherein the agent is an ICE antagonist.

14530. The method of item 14456 wherein the agent is an IRAK antagonist.

14531. The method of item 14456 wherein the agent is an IL-4 agonist.

14532. The method of item 14456 wherein the agent is an immunomodulatory agent.

14533. The method of item 14456 wherein the agent is sirolimus or an analogue or derivative thereof.

14534. The method of item 14456 wherein the agent is not sirolimus.

14535. The method of item 14456 wherein the agent is everolimus or an analogue or derivative thereof.

14536. The method of item 14456 wherein the agent is tacrolimus or an analogue or derivative thereof.

14537. The method of item 14456 wherein the agent is not tacrolimus.

14538. The method of item 14456 wherein the agent is biolmus or an analogue or derivative thereof.

14539. The method of item 14456 wherein the agent is tresperimus or an analogue or derivative thereof.

14540. The method of item 14456 wherein the agent is auranofin or an analogue or derivative thereof.

14541. The method of item 14456 wherein the agent is 27-0-demethylrapamycin or an analogue or derivative thereof.

14542. The method of item 14456 wherein the agent is gusperimus or an analogue or derivative thereof.

14543. The method of item 14456 wherein the agent is pimecrolimus or an analogue or derivative thereof.

14544. The method of item 14456 wherein the agent is ABT-578 or an analogue or derivative thereof.

14545. The method of item 14456 wherein the agent is an inosine monophosphate dehydrogenase (IMPDH) inhibitor.

14546. The method of item 14456 wherein the agent is an IMPDH inhibitor, wherein the IMPDH inhibitor is mycophenolic acid or an analogue or derivative thereof.

14547. The method of item 14456 wherein the agent is an IMPDH inhibitor, wherein the IMPDH inhibitor is 1-alpha-25 dihydroxy vitamin D₃ or an analogue or derivative thereof.

14548. The method of item 14456 wherein the agent is a leukotriene inhibitor.

14549. The method of item 14456 wherein the agent is a MCP-1 antagonist.

14550. The method of item 14456 wherein the agent is a MMP inhibitor.

14551. The method of item 14456 wherein the agent is an NF kappa B inhibitor.

14552. The method of item 14456 wherein the agent is an NF kappa B inhibitor, wherein the NF kappa B inhibitor is Bay 11-7082.

14553. The method of item 14456 wherein the agent is an NO agonist.

14554. The method of item 14456 wherein the agent is a p38 MAP kinase inhibitor.

14555. The method of item 14456 wherein the agent is a p38 MAP kinase inhibitor, wherein the p38 MAP kinase inhibitor is SB 202190.

14556. The method of item 14456 wherein the agent is a phosphodiesterase inhibitor.

14557. The method of item 14456 wherein the agent is a TGF beta inhibitor.

14558. The method of item 14456 wherein the agent is a thromboxane A2 antagonist.

14559. The method of item 14456 wherein the agent is a TNFa antagonist.

14560. The method of item 14456 wherein the agent is a TACE inhibitor.

14561. The method of item 14456 wherein the agent is a tyrosine kinase inhibitor.

14562. The method of item 14456 wherein the agent is a vitronectin inhibitor.

14563. The method of item 14456 wherein the agent is a fibroblast growth factor inhibitor.

14564. The method of item 14456 wherein the agent is a protein kinase inhibitor.

14565. The method of item 14456 wherein the agent is a PDGF receptor kinase inhibitor.

14566. The method of item 14456 wherein the agent is an endothelial growth factor receptor kinase inhibitor.

14567. The method of item 14456 wherein the agent is a retinoic acid receptor antagonist.

14568. The method of item 14456 wherein the agent is a platelet derived growth factor receptor kinase inhibitor.

14569. The method of item 14456 wherein the agent is a fibronogin antagonist.

14570. The method of item 14456 wherein the agent is an antimycotic agent.

14571. The method of item 14456 wherein the agent is an antimycotic agent, wherein the antimycotic agent is sulconizole.

14572. The method of item 14456 wherein the agent is a bisphosphonate.

14573. The method of item 14456 wherein the agent is a phospholipase A1 inhibitor.

14574. The method of item 14456 wherein the agent is a histamine H1/H2/H3 receptor antagonist.

14575. The method of item 14456 wherein the agent is a macrolide antibiotic.

14576. The method of item 14456 wherein the agent is a GPIIb/IIIa receptor antagonist.

14577. The method of item 14456 wherein the agent is an endothelin receptor antagonist.

14578. The method of item 14456 wherein the agent is a peroxisome proliferator-activated receptor agonist.

14579. The method of item 14456 wherein the agent is an estrogen receptor agent.

14580. The method of item 14456 wherein the agent is a somastostatin analogue.

14581. The method of item 14456 wherein the agent is a neurokinin 1 antagonist.

14582. The method of item 14456 wherein the agent is a neurokinin 3 antagonist.

14583. The method of item 14456 wherein the agent is a VLA-4 antagonist.

14584. The method of item 14456 wherein the agent is an osteoclast inhibitor.

14585. The method of item 14456 wherein the agent is a DNA topoisomerase ATP hydrolyzing inhibitor.

14586. The method of item 14456 wherein the agent is an angiotensin I converting enzyme inhibitor.

14587. The method of item 14456 wherein the agent is an angiotensin II antagonist.

14588. The method of item 14456 wherein the agent is an enkephalinase inhibitor.

14589. The method of item 14456 wherein the agent is a peroxisome proliferator-activated receptor gamma agonist insulin sensitizer.

14590. The method of item 14456 wherein the agent is a protein kinase C inhibitor.

14591. The method of item 14456 wherein the agent is a ROCK (rho-associated kinase) inhibitor.

14592. The method of item 14456 wherein the agent is a CXCR3 inhibitor.

14593. The method of item 14456 wherein the agent is an Itk inhibitor.

14594. The method of item 14456 wherein the agent is a cytosolic phospholipase A₂-alpha inhibitor.

14595. The method of item 14456 wherein the agent is a PPAR agonist.

14596. The method of item 14456 wherein the agent is an immunosuppressant.

14597. The method of item 14456 wherein the agent is an Erb inhibitor.

14598. The method of item 14456 wherein the agent is an apoptosis agonist.

14599. The method of item 14456 wherein the agent is a lipocortin agonist.

14600. The method of item 14456 wherein the agent is a VCAM-1 antagonist.

14601. The method of item 14456 wherein the agent is a collagen antagonist.

14602. The method of item 14456 wherein the agent is an alpha 2 integrin antagonist.

14603. The method of item 14456 wherein the agent is a TNF alpha inhibitor.

14604. The method of item 14456 wherein the agent is a nitric oxide inhibitor.

14605. The method of item 14456 wherein the agent is a cathepsin inhibitor.

14606. The method of item 14456 wherein the agent is not an anti-inflammatory agent.

14607. The method of item 14456 wherein the agent is not a steroid.

14608. The method of item 14456 wherein the agent is not a glucocorticosteroid.

14609. The method of item 14456 wherein the agent is not dexamethasone.

14610. The method of item 14456 wherein the agent is not an anti-infective agent.

14611. The method of item 14456 wherein the agent is not an antibiotic.

14612. The method of item 14456 wherein the agent is not an anti-fungal agent.

14613. The method of item 14456, wherein the composition comprises a polymer.

14614. The method of item 14456, wherein the composition comprises a polymeric carrier.

14615. The method of item 14456 wherein the anti-scarring agent inhibits adhesion between the device and a host into which the device is implanted.

14616. The method of item 14456 wherein the device delivers the anti-scarring agent locally to tissue proximate to the device.

14617. The method of item 14456 wherein the device has a coating that comprises the anti-scarring agent.

14618. The method of item 14456, wherein the device has a coating that comprises the agent and is disposed on a surface of the implant.

14619. The method of item 14456, wherein the device has a coating that comprises the agent and directly contacts the implant.

14620. The method of item 14456, wherein the device has a coating that comprises the agent and indirectly contacts the implant.

14621. The method of item 14456, wherein the device has a coating that comprises the agent and partially covers the implant.

14622. The method of item 14456, wherein the device has a coating that comprises the agent and completely covers the implant.

14623. The method of item 14456, wherein the device has a uniform coating.

14624. The method of item 14456, wherein the device has a non-uniform coating.

14625. The method of item 14456, wherein the device has a discontinuous coating.

14626. The method of item 14456, wherein the device has a patterned coating.

14627. The method of item 14456, wherein the device has a coating with a thickness of 100 μm or less.

14628. The method of item 14456, wherein the device has a coating with a thickness of 10 μm or less.

14629. The method of item 14456, wherein the device has a coating, and the coating adheres to the surface of the implant upon deployment of the implant.

14630. The method of item 14456, wherein the device has a coating, and wherein the coating is stable at room temperature for a period of 1 year.

14631. The method of item 14456, wherein the device has a coating, and wherein the anti-scarring agent is present in the coating in an amount ranging between about 0.0001% to about 1% by weight.

14632. The method of item 14456, wherein the device has a coating, and wherein the anti-scarring agent is present in the coating in an amount ranging between about 1% to about 10% by weight.

14633. The method of item 14456, wherein the device has a coating, and wherein the anti-scarring agent is present in the coating in an amount ranging between about 10% to about 25% by weight.

14634. The method of item 14456, wherein the device has a coating, and wherein the anti-scarring agent is present in the coating in an amount ranging between about 25% to about 70% by weight.

14635. The method of item 14456, wherein the device has a coating, and wherein the coating further comprises a polymer.

14636. The method of item 14456, wherein the device has a first coating having a first composition and a second coating having a second composition.

14637. The method of item 14456, wherein the device has a first coating having a first composition and a second coating having a second composition, wherein the first composition and the second composition are different.

14638. The method of item 14456, wherein the composition comprises a polymer.

14639. The method of item 14456, wherein the composition comprises a polymeric carrier.

14640. The method of item 14456, wherein the composition comprises a polymeric carrier, and wherein the polymeric carrier comprises a copolymer.

14641. The method of item 14456, wherein the composition comprises a polymeric carrier, and wherein the polymeric carrier comprises a block copolymer.

14642. The method of item 14456, wherein the composition comprises a polymeric carrier, and wherein the polymeric carrier comprises a random copolymer.

14643. The method of item 14456, wherein the composition comprises a polymeric carrier, and wherein the polymeric carrier comprises a biodegradable polymer.

14644. The method of item 14456, wherein the composition comprises a polymeric carrier, and wherein the polymeric carrier comprises a non-biodegradable polymer.

14645. The method of item 14456, wherein the composition comprises a polymeric carrier, and wherein the polymeric carrier comprises a hydrophilic polymer.

14646. The method of item 14456, wherein the composition comprises a polymeric carrier, and wherein the polymeric carrier comprises a hydrophobic polymer.

14647. The method of item 14456, wherein the composition comprises a polymeric carrier, and wherein the polymeric carrier comprises a polymer having hydrophilic domains.

14648. The method of item 14456, wherein the composition comprises a polymeric carrier, and wherein the polymeric carrier comprises a polymer having hydrophobic domains.

14649. The method of item 14456, wherein the composition comprises a polymeric carrier, and wherein the polymeric carrier comprises a non-conductive polymer.

14650. The method of item 14456, wherein the composition comprises a polymeric carrier, and wherein the polymeric carrier comprises an elastomer.

14651. The method of item 14456, wherein the composition comprises a polymeric carrier, and wherein the polymeric carrier comprises a hydrogel.

14652. The method of item 14456, wherein the composition comprises a polymeric carrier, and wherein the polymeric carrier comprises a silicone polymer.

14653. The method of item 14456, wherein the composition comprises a polymeric carrier, and wherein the polymeric carrier comprises a hydrocarbon polymer.

14654. The method of item 14456, wherein the composition comprises a polymeric carrier, and wherein the polymeric carrier comprises a styrene-derived polymer.

14655. The method of item 14456, wherein the composition comprises a polymeric carrier, and wherein the polymeric carrier comprises a butadiene polymer.

14656. The method of item 14456, wherein the composition comprises a polymeric carrier, and wherein the polymeric carrier comprises a macromer.

14657. The method of item 14456, wherein the composition comprises a polymeric carrier, and wherein the polymeric carrier comprises a poly(ethylene glycol)polymer.

14658. The method of item 14456 wherein the composition comprises a polymeric carrier, and wherein the polymeric carrier comprises an amorphous polymer.

14659. The method of item 14456, wherein the device comprises a lubricious coating.

14660. The method of item 14456 wherein the anti-scarring agent is located within pores or holes of the device.

14661. The method of item 14456 wherein the anti-scarring agent is located within a channel, lumen, or divet of the device.

14662. The method of item 14456, wherein the device comprises a second pharmaceutically active agent.

14663. The method of item 14456 wherein the device comprises an anti-inflammatory agent.

14664. The method of item 14456 wherein the device comprises an agent that inhibits infection.

14665. The method of item 14456 wherein the device comprises an agent that inhibits infection, and wherein the agent is an anthracycline.

14666. The method of item 14456 wherein the device comprises an agent that inhibits infection, and wherein the agent is doxorubicin.

14667. The method of item 14456 wherein the device comprises an agent that inhibits infection, and wherein the agent is mitoxantrone.

14668. The method of item 14456 wherein the device comprises an agent that inhibits infection, and wherein the agent is a fluoropyrimidine.

14669. The method of item 14456 wherein the device comprises an agent that inhibits infection, and wherein the agent is 5-fluorouracil (5-FU).

14670. The method of item 14456 wherein the device comprises an agent that inhibits infection, and wherein the agent is a folic acid antagonist.

14671. The method of item 14456 wherein the device comprises an agent that inhibits infection, and wherein the agent is methotrexate.

14672. The method of item 14456 wherein the device comprises an agent that inhibits infection, and wherein the agent is a podophylotoxin.

14673. The method of item 14456 wherein the device comprises an agent that inhibits infection, and wherein the agent is etoposide.

14674. The method of item 14456 wherein the device comprises an agent that inhibits infection, and wherein the agent is a camptothecin.

14675. The method of item 14456 wherein the device comprises an agent that inhibits infection, and wherein the agent is a hydroxyurea.

14676. The method of item 14456 wherein the device comprises an agent that inhibits infection, and wherein the agent is a platinum complex.

14677. The method of item 14456 wherein the device comprises an agent that inhibits infection, and wherein the agent is cisplatin.

14678. The method of item 14456, further comprising an anti-thrombotic agent.

14679. The method of item 14456 wherein the device comprises a visualization agent.

14680. The method of item 14456 wherein the device comprises a visualization agent, wherein the visualization agent is a radiopaque material, and wherein the radiopaque material comprises a metal, a halogenated compound, or a barium containing compound.

14681. The method of item 14456 wherein the device comprises a visualization agent, wherein the visualization agent is a radiopaque material, and wherein the radiopaque material comprises barium, tantalum, or technetium.

14682. The method of item 14456 wherein the device comprises a visualization agent, and wherein the visualization agent is a MRI responsive material.

14683. The method of item 14456 wherein the device comprises a visualization agent, and wherein the visualization agent comprises a gadolinium chelate.

14684. The method of item 14456 wherein the device comprises a visualization agent, and wherein the visualization agent comprises iron, magnesium, manganese, copper, or chromium.

14685. The method of item 14456 wherein the device comprises a visualization agent, and wherein the visualization agent comprises an iron oxide compound.

14686. The method of item 14456 wherein the device comprises a visualization agent, and wherein the visualization agent comprises a dye, pigment, or colorant.

14687. The method of item 14456 wherein the device comprises an echogenic material.

14688. The method of item 14456 wherein the device comprises an echogenic material, and wherein the echogenic material is in the form of a coating.

14689. The method of item 14456 wherein the device is sterile.

14690. The method of item 14456 wherein the anti-scarring agent is released into tissue in the vicinity of the device after deployment of the device.

14691. The method of item 14456 wherein the anti-scarring agent is released into tissue in the vicinity of the device after deployment of the device, and wherein the tissue is connective tissue.

14692. The method of item 14456 wherein the anti-scarring agent is released into tissue in the vicinity of the device after deployment of the device, and wherein the tissue is muscle tissue.

14693. The method of item 14456 wherein the anti-scarring agent is released into tissue in the vicinity of the device after deployment of the device, and wherein the tissue is nerve tissue.

14694. The method of item 14456 wherein the anti-scarring agent is released into tissue in the vicinity of the device after deployment of the device, and wherein the tissue is epithelium tissue.

14695. The method of item 14456 wherein the anti-scarring agent is released in effective concentrations from the device over a period ranging from the time of deployment of the device to about 1 year.

14696. The method of item 14456 wherein the anti-scarring agent is released in effective concentrations from the device over a period ranging from about 1 month to 6 months.

14697. The method of item 14456 wherein the anti-scarring agent is released in effective concentrations from the device over a period ranging from about 1-90 days.

14698. The method of item 14456 wherein the anti-scarring agent is released in effective concentrations from the device at a constant rate.

14699. The method of item 14456 wherein the anti-scarring agent is released in effective concentrations from the device at an increasing rate.

14700. The method of item 14456 wherein the anti-scarring agent is released in effective concentrations from the device at a decreasing rate.

14701. The method of item 14456 wherein the anti-scarring agent is released in effective concentrations from the composition comprising the anti-scarring agent by diffusion over a period ranging from the time of deployment of the device to about 90 days.

14702. The method of item 14456 wherein the anti-scarring agent is released in effective concentrations from the composition comprising the anti-scarring agent by erosion of the composition over a period ranging from the time of deployment of the device to about 90 days.

14703. The method of item 14456 wherein the device comprises about 0.01 μg to about 10 μg of the anti-scarring agent.

14704. The method of item 14456 wherein the device comprises about 10 μg to about 10 mg of the anti-scarring agent.

14705. The method of item 14456 wherein the device comprises about 10 mg to about 250 mg of the anti-scarring agent.

14706. The method of item 14456 wherein the device comprises about 250 mg to about 1000 mg of the anti-scarring agent.

14707. The method of item 14456 wherein the device comprises about 1000 mg to about 2500 mg of the anti-scarring agent.

14708. The method of item 14456 wherein a surface of the device comprises less than 0.01 μg of the anti-scarring agent per mm² of device surface to which the anti-scarring agent is applied.

14709. The method of item 14456 wherein a surface of the device comprises about 0.01 μg to about 1 μg of the anti-scarring agent per mm² of device surface to which the anti-scarring agent is applied.

14710. The method of item 14456 wherein a surface of the device comprises about 1 μg to about 10 μg of the anti-scarring agent per mm² of device surface to which the anti-scarring agent is applied.

14711. The method of item 14456 wherein a surface of the device comprises about 10 μg to about 250 μg of the anti-scarring agent per mm² of device surface to which the anti-scarring agent is applied.

14712. The method of item 14456 wherein a surface of the device comprises about 250 μg to about 1000 μg of the anti-scarring agent of anti-scarring agent per mm² of device surface to which the anti-scarring agent is applied.

14713. The method of item 14456 wherein a surface of the device comprises about 1000 μg to about 2500 μg of the anti-scarring agent per mm² of device surface to which the anti-scarring agent is applied.

0.14714. The method of item 14456 wherein the combining is performed by direct affixing the agent or the composition to the implant.

14715. The method of item 14456 wherein the combining is performed by spraying the agent or the component onto the implant.

14716. The method of item 14456 wherein the combining is performed by electrospraying the agent or the composition onto the implant.

14717. The method of item 14456 wherein the combining is performed by dipping the implant into a solution comprising the agent or the composition.

14718. The method of item 14456 wherein the combining is performed by covalently attaching the agent or the composition to the implant.

14719. The method of item 14456 wherein the combining is performed by non-covalently attaching the agent or the composition to the implant.

14720. The method of item 14456 wherein the combining is performed by coating the implant with a substance that contains the agent or the composition.

14721. The method of item 14456 wherein the combining is performed by coating the implant with a substance that absorbs the agent.

14722. The method of item 14456 wherein the combining is performed by interweaving a thread composed of, or coated with, the agent or the composition.

14723. The method of item 14456 wherein the combining is performed by covering all the implant with a sleeve that contains the agent or the composition.

14724. The method of item 14456 wherein the combining is performed by covering a portion of the implant with a sleeve that contains the agent or the composition.

14725. The method of item 14456 wherein the combining is performed by covering all the implant with a cover that contains the agent or the composition.

14726. The method of item 14456 wherein the combining is performed by covering a portion of the implant with a cover that contains the agent or the composition.

14727. The method of item 14456 wherein the combining is performed by covering all the implant with an electrospun fabric that contains the agent or the composition.

14728. The method of item 14456 wherein the combining is performed by covering a portion of the implant with an electrospun fabric that contains the agent or the composition.

14729. The method of item 14456 wherein the combining is performed by covering all the implant with a mesh that contains the agent or the composition.

14730. The method of item 14456 wherein the combining is performed by covering a portion of the implant with a mesh that contains the agent or the composition.

14731. The method of item 14456 wherein the combining is performed by constructing all the implant with the agent or the composition.

14732. The method of item 14456 wherein the combining is performed by constructing a portion of the implant with the agent or the composition.

14733. The method of item 14456 wherein the combining is performed by impregnating the implant with the agent or the composition.

14734. The method of item 14456 wherein the combining is performed by constructing all of the implant from a degradable polymer that releases the agent.

14735. The method of item 14456 wherein the combining is performed by constructing a portion of the implant from a degradable polymer that releases the agent.

14736. The method of item 14456 wherein the combining is performed by dipping the implant into a solution that comprise the agent and an inert solvent for the implant.

14737. The method of item 14456 wherein the combining is performed by dipping the implant into a solution that comprises the agent and a solvent that will swill the implant.

14738. The method of item 14456 wherein the combining is performed by dipping the implant into a solution that comprises the agent and a solvent that will dissolve the implant.

14739. The method of item 14456 wherein the combining is performed by dipping the implant into a solution that comprises the agent, a polymer and an inert solvent for the implant.

14740. The method of item 14456 wherein the combining is performed by dipping the implant into a solution that comprises the agent, a polymer and a solvent that will swill the implant.

14741. The method of item 14456 wherein the combining is performed by dipping the implant into a solution that comprises the agent, a polymer and a solvent that will dissolve the implant.

14742. The method of item 14456 wherein the combining is performed by spraying the implant into a solution that comprises the agent and an inert solvent for the implant.

14743. The method of item 14456 wherein the combining is performed by spraying the implant into a solution that comprises the agent and a solvent that will swill the implant.

14744. The method of item 14456 wherein the combining is performed by spraying the implant into a solution that comprises the agent and a solvent that will dissolve the implant.

14745. The method of item 14456 wherein the combining is performed by spraying the implant into a solution that comprises the agent, a polymer and an inert solvent for the implant.

14746. The method of item 14456 wherein the combining is performed by spraying the implant into a solution that comprises the agent, a polymer and a solvent that will swill the implant.

14747. The method of item 14456 wherein the combining is performed by spraying the implant into a solution that comprises the agent, a polymer and a solvent that will dissolve the implant.

14748. A method of making a medical device comprising: combining a tracheostomy tube (i.e., an implant) and an anti-scarring agent or a composition comprising an anti-scarring agent, wherein the agent inhibits scarring between the device and a host into which the device is implanted.

14749. The method of item 14748 wherein the agent inhibits cell regeneration.

14750. The method of item 14748 wherein the agent inhibits angiogenesis.

14751. The method of item 14748 wherein the agent inhibits fibroblast migration.

14752. The method of item 14748 wherein the agent inhibits fibroblast proliferation.

14753. The method of item 14748 wherein the agent inhibits deposition of extracellular matrix.

14754. The method of item 14748 wherein the agent inhibits tissue remodeling.

14755. The method of item 14748 wherein the agent is an angiogenesis inhibitor.

14756. The method of item 14748 wherein the agent is a 5-lipoxygenase inhibitor or antagonist.

14757. The method of item 14748 wherein the agent is a chemokine receptor antagonist.

14758. The method of item 14748 wherein the agent is a cell cycle inhibitor.

14759. The method of item 14748 wherein the agent is a taxane.

14760. The method of item 14748 wherein the agent is an anti-microtubule agent.

14761. The method of item 14748 wherein the agent is paclitaxel.

14762. The method of item 14748 wherein the agent is not paclitaxel.

14763. The method of item 14748 wherein the agent is an analogue or derivative of paclitaxel.

14764. The method of item 14748 wherein the agent is a vinca alkaloid.

14765. The method of item 14748 wherein the agent is camptothecin or an analogue or derivative thereof.

14766. The method of item 14748 wherein the agent is a podophyllotoxin.

14767. The method of item 14748 wherein the agent is a podophyllotoxin, wherein the podophyllotoxin is etoposide or an analogue or derivative thereof.

14768. The method of item 14748 wherein the agent is an anthracycline.

14769. The method of item 14748 wherein the agent is an anthracycline, wherein the anthracycline is doxorubicin or an analogue or derivative thereof.

14770. The method of item 14748 wherein the agent is an anthracycline, wherein the anthracycline is mitoxantrone or an analogue or derivative thereof.

14771. The method of item 14748 wherein the agent is a platinum compound.

14772. The method of item 14748 wherein the agent is a nitrosourea.

14773. The method of item 14748 wherein the agent is a nitroimidazole.

14774. The method of item 14748 wherein the agent is a folic acid antagonist.

14775. The method of item 14748 wherein the agent is a cytidine analogue.

14776. The method of item 14748 wherein the agent is a pyrimidine analogue.

14777. The method of item 14748 wherein the agent is a fluoropyrimidine analogue.

14778. The method of item 14748 wherein the agent is a purine analogue.

14779. The method of item 14748 wherein the agent is a nitrogen mustard or an analogue or derivative thereof.

14780. The method of item 14748 wherein the agent is a hydroxyurea.

14781. The method of item 14748 wherein the agent is a mytomicin or an analogue or derivative thereof.

14782. The method of item 14748 wherein the agent is an alkyl sulfonate.

14783. The method of item 14748 wherein the agent is a benzamide or an analogue or derivative thereof.

14784. The method of item 14748 wherein the agent is a nicotinamide or an analogue or derivative thereof.

14785. The method of item 14748 wherein the agent is a halogenated sugar or an analogue or derivative thereof.

14786. The method of item 14748 wherein the agent is a DNA alkylating agent.

14787. The method of item 14748 wherein the agent is an anti-microtubule agent.

14788. The method of item 14748 wherein the agent is a topoisomerase inhibitor.

14789. The method of item 14748 wherein the agent is a DNA cleaving agent.

14790. The method of item 14748 wherein the agent is an antimetabolite.

14791. The method of item 14748 wherein the agent inhibits adenosine deaminase.

14792. The method of item 14748 wherein the agent inhibits purine ring synthesis.

14793. The method of item 14748 wherein the agent is a nucleotide interconversion inhibitor.

14794. The method of item 14748 wherein the agent inhibits dihydrofolate reduction.

14795. The method of item 14748 wherein the agent blocks thymidine monophosphate.

14796. The method of item 14748 wherein the agent causes DNA damage.

14797. The method of item 14748 wherein the agent is a DNA intercalation agent.

14798. The method of item 14748 wherein the agent is a RNA synthesis inhibitor.

14799. The method of item 14748 wherein the agent is a pyrimidine synthesis inhibitor.

14800. The method of item 14748 wherein the agent inhibits ribonucleotide synthesis or function.

14801. The method of item 14748 wherein the agent inhibits thymidine monophosphate synthesis or function.

14802. The method of item 14748 wherein the agent inhibits DNA synthesis.

14803. The method of item 14748 wherein the agent causes DNA adduct formation.

14804. The method of item 14748 wherein the agent inhibits protein synthesis.

14805. The method of item 14748 wherein the agent inhibits microtubule function.

14806. The method of item 14748 wherein the agent is a cyclin dependent protein kinase inhibitor.

14807. The method of item 14748 wherein the agent is an epidermal growth factor kinase inhibitor.

14808. The method of item 14748 wherein the agent is an elastase inhibitor.

14809. The method of item 14748 wherein the agent is a factor Xa inhibitor.

14810. The method of item 14748 wherein the agent is a farnesyltransferase inhibitor.

14811. The method of item 14748 wherein the agent is a fibrinogen antagonist.

14812. The method of item 14748 wherein the agent is a guanylate cyclase stimulant.

14813. The method of item 14748 wherein the agent is a heat shock protein 90 antagonist.

14814. The method of item 14748 wherein the agent is a heat shock protein 90 antagonist, wherein the heat shock protein 90 antagonist is geldanamycin or an analogue or derivative thereof.

14815. The method of item 14748 wherein the agent is a guanylate cyclase stimulant.

14816. The method of item 14748 wherein the agent is a HMGCoA reductase inhibitor.

14817. The method of item 14748 wherein the agent is a HMGCoA reductase inhibitor, wherein the HMGCoA reductase inhibitor is simvastatin or an analogue or derivative thereof.

14818. The method of item 14748 wherein the agent is a hydroorotate dehydrogenase inhibitor.

14819. The method of item 14748 wherein the agent is an IKK2 inhibitor.

14820. The method of item 14748 wherein the agent is an IL-1 antagonist.

14821. The method of item 14748 wherein the agent is an ICE antagonist.

14822. The method of item 14748 wherein the agent is an IRAK antagonist.

14823. The method of item 14748 wherein the agent is an IL-4 agonist.

14824. The method of item 14748 wherein the agent is an immunomodulatory agent.

14825. The method of item 14748 wherein the agent is sirolimus or an analogue or derivative thereof.

14826. The method of item 14748 wherein the agent is not sirolimus.

14827. The method of item 14748 wherein the agent is everolimus or an analogue or derivative thereof.

14828. The method of item 14748 wherein the agent is tacrolimus or an analogue or derivative thereof.

14829. The method of item 14748 wherein the agent is not tacrolimus.

14830. The method of item 14748 wherein the agent is biolmus or an analogue or derivative thereof.

14831. The method of item 14748 wherein the agent is tresperimus or an analogue or derivative thereof.

14832. The method of item 14748 wherein the agent is auranofin or an analogue or derivative thereof.

14833. The method of item 14748 wherein the agent is 27-0-demethylrapamycin or an analogue or derivative thereof.

14834. The method of item 14748 wherein the agent is gusperimus or an analogue or derivative thereof.

14835. The method of item 14748 wherein the agent is pimecrolimus or an analogue or derivative thereof.

14836. The method of item 14748 wherein the agent is ABT-578 or an analogue or derivative thereof.

14837. The method of item 14748 wherein the agent is an inosine monophosphate dehydrogenase (IMPDH) inhibitor.

14838. The method of item 14748 wherein the agent is an IMPDH inhibitor, wherein the IMPDH inhibitor is mycophenolic acid or an analogue or derivative thereof.

14839. The method of item 14748 wherein the agent is an IMPDH inhibitor, wherein the IMPDH inhibitor is 1-alpha-25 dihydroxy vitamin D₃ or an analogue or derivative thereof.

14840. The method of item 14748 wherein the agent is a leukotriene inhibitor.

14841. The method of item 14748 wherein the agent is a MCP-1 antagonist.

14842. The method of item 14748 wherein the agent is a MMP inhibitor.

14843. The method of item 14748 wherein the agent is an NF kappa B inhibitor.

14844. The method of item 14748 wherein the agent is an NF kappa B inhibitor, wherein the NF kappa B inhibitor is Bay 11-7082.

14845. The method of item 14748 wherein the agent is an NO agonist.

14846. The method of item 14748 wherein the agent is a p38 MAP kinase inhibitor.

14847. The method of item 14748 wherein the agent is a p38 MAP kinase inhibitor, wherein the p38 MAP kinase inhibitor is SB 202190.

14848. The method of item 14748 wherein the agent is a phosphodiesterase inhibitor.

14849. The method of item 14748 wherein the agent is a TGF beta inhibitor.

14850. The method of item 14748 wherein the agent is a thromboxane A2 antagonist.

14851. The method of item 14748 wherein the agent is a TNFa antagonist.

14852. The method of item 14748 wherein the agent is a TACE inhibitor.

14853. The method of item 14748 wherein the agent is a tyrosine kinase inhibitor.

14854. The method of item 14748 wherein the agent is a vitronectin inhibitor.

14855. The method of item 14748 wherein the agent is a fibroblast growth factor inhibitor.

14856. The method of item 14748 wherein the agent is a protein kinase inhibitor.

14857. The method of item 14748 wherein the agent is a PDGF receptor kinase inhibitor.

14858. The method of item 14748 wherein the agent is an endothelial growth factor receptor kinase inhibitor.

14859. The method of item 14748 wherein the agent is a retinoic acid receptor antagonist.

14860. The method of item 14748 wherein the agent is a platelet derived growth factor receptor kinase inhibitor.

14861. The method of item 14748 wherein the agent is a fibronogin antagonist.

14862. The method of item 14748 wherein the agent is an antimycotic agent.

14863. The method of item 14748 wherein the agent is an antimycotic agent, wherein the antimycotic agent is sulconizole.

14864. The method of item 14748 wherein the agent is a bisphosphonate.

14865. The method of item 14748 wherein the agent is a phospholipase A1 inhibitor.

14866. The method of item 14748 wherein the agent is a histamine H1/H2/H3 receptor antagonist.

14867. The method of item 14748 wherein the agent is a macrolide antibiotic.

14868. The method of item 14748 wherein the agent is a GPIIb/IIIa receptor antagonist.

14869. The method of item 14748 wherein the agent is an endothelin receptor antagonist.

14870. The method of item 14748 wherein the agent is a peroxisome proliferator-activated receptor agonist.

14871. The method of item 14748 wherein the agent is an estrogen receptor agent.

14872. The method of item 14748 wherein the agent is a somastostatin analogue.

14873. The method of item 14748 wherein the agent is a neurokinin 1 antagonist.

14874. The method of item 14748 wherein the agent is a neurokinin 3 antagonist.

14875. The method of item 14748 wherein the agent is a VLA-4 antagonist.

14876. The method of item 14748 wherein the agent is an osteoclast inhibitor.

14877. The method of item 14748 wherein the agent is a DNA topoisomerase ATP hydrolyzing inhibitor.

14878. The method of item 14748 wherein the agent is an angiotensin I converting enzyme inhibitor.

14879. The method of item 14748 wherein the agent is an angiotensin II antagonist.

14880. The method of item 14748 wherein the agent is an enkephalinase inhibitor.

14881. The method of item 14748 wherein the agent is a peroxisome proliferator-activated receptor gamma agonist insulin sensitizer.

14882. The method of item 14748 wherein the agent is a protein kinase C inhibitor.

14883. The method of item 14748 wherein the agent is a ROCK (rho-associated kinase) inhibitor.

14884. The method of item 14748 wherein the agent is a CXCR3 inhibitor.

14885. The method of item 14748 wherein the agent is an Itk inhibitor.

14886. The method of item 14748 wherein the agent is a cytosolic phospholipase A₂-alpha inhibitor.

14887. The method of item 14748 wherein the agent is a PPAR agonist.

14888. The method of item 14748 wherein the agent is an immunosuppressant.

14889. The method of item 14748 wherein the agent is an Erb inhibitor.

14890. The method of item 14748 wherein the agent is an apoptosis agonist.

14891. The method of item 14748 wherein the agent is a lipocortin agonist.

14892. The method of item 14748 wherein the agent is a VCAM-1 antagonist.

14893. The method of item 14748 wherein the agent is a collagen antagonist.

14894. The method of item 14748 wherein the agent is an alpha 2 integrin antagonist.

14895. The method of item 14748 wherein the agent is a TNF alpha inhibitor.

14896. The method of item 14748 wherein the agent is a nitric oxide inhibitor.

14897. The method of item 14748 wherein the agent is a cathepsin inhibitor.

14898. The method of item 14748 wherein the agent is not an anti-inflammatory agent.

14899. The method of item 14748 wherein the agent is not a steroid.

14900. The method of item 14748 wherein the agent is not a glucocorticosteroid.

14901. The method of item 14748 wherein the agent is not dexamethasone.

14902. The method of item 14748 wherein the agent is not an anti-infective agent.

14903. The method of item 14748 wherein the agent is not an antibiotic.

14904. The method of item 14748 wherein the agent is not an anti-fungal agent.

14905. The method of item 14748, wherein the composition comprises a polymer.

14906. The method of item 14748, wherein the composition comprises a polymeric carrier.

14907. The method of item 14748 wherein the anti-scarring agent inhibits adhesion between the device and a host into which the device is implanted.

14908. The method of item 14748 wherein the device delivers the anti-scarring agent locally to tissue proximate to the device.

14909. The method of item 14748 wherein the device has a coating that comprises the anti-scarring agent.

14910. The method of item 14748, wherein the device has a coating that comprises the agent and is disposed on a surface of the implant.

14911. The method of item 14748, wherein the device has a coating that comprises the agent and directly contacts the implant.

14912. The method of item 14748, wherein the device has a coating that comprises the agent and indirectly contacts the implant.

14913. The method of item 14748, wherein the device has a coating that comprises the agent and partially covers the implant.

14914. The method of item 14748, wherein the device has a coating that comprises the agent and completely covers the implant.

14915. The method of item 14748, wherein the device has a uniform coating.

14916. The method of item 14748, wherein the device has a non-uniform coating.

14917. The method of item 14748, wherein the device has a discontinuous coating.

14918. The method of item 14748, wherein the device has a patterned coating.

14919. The method of item 14748, wherein the device has a coating with a thickness of 100 μm or less.

14920. The method of item 14748, wherein the device has a coating with a thickness of 10 μm or less.

14921. The method of item 14748, wherein the device has a coating, and the coating adheres to the surface of the implant upon deployment of the implant.

14922. The method of item 14748, wherein the device has a coating, and wherein the coating is stable at room temperature for a period of 1 year.

14923. The method of item 14748, wherein the device has a coating, and wherein the anti-scarring agent is present in the coating in an amount ranging between about 0.0001% to about 1% by weight.

14924. The method of item 14748, wherein the device has a coating, and wherein the anti-scarring agent is present in the coating in an amount ranging between about 1% to about 10% by weight.

14925. The method of item 14748, wherein the device has a coating, and wherein the anti-scarring agent is present in the coating in an amount ranging between about 10% to about 25% by weight.

14926. The method of item 14748, wherein the device has a coating, and wherein the anti-scarring agent is present in the coating in an amount ranging between about 25% to about 70% by weight.

14927. The method of item 14748, wherein the device has a coating, and wherein the coating further comprises a polymer.

14928. The method of item 14748, wherein the device has a first coating having a first composition and a second coating having a second composition.

14929. The method of item 14748, wherein the device has a first coating having a first composition and a second coating having a second composition, wherein the first composition and the second composition are different.

14930. The method of item 14748, wherein the composition comprises a polymer.

14931. The method of item 14748, wherein the composition comprises a polymeric carrier.

14932. The method of item 14748, wherein the composition comprises a polymeric carrier, and wherein the polymeric carrier comprises a copolymer.

14933. The method of item 14748, wherein the composition comprises a polymeric carrier, and wherein the polymeric carrier comprises a block copolymer.

14934. The method of item 14748, wherein the composition comprises a polymeric carrier, and wherein the polymeric carrier comprises a random copolymer.

14935. The method of item 14748, wherein the composition comprises a polymeric carrier, and wherein the polymeric carrier comprises a biodegradable polymer.

14936. The method of item 14748, wherein the composition comprises a polymeric carrier, and wherein the polymeric carrier comprises a non-biodegradable polymer.

14937. The method of item 14748, wherein the composition comprises a polymeric carrier, and wherein the polymeric carrier comprises a hydrophilic polymer.

14938. The method of item 14748, wherein the composition comprises a polymeric carrier, and wherein the polymeric carrier comprises a hydrophobic polymer.

14939. The method of item 14748, wherein the composition comprises a polymeric carrier, and wherein the polymeric carrier comprises a polymer having hydrophilic domains.

14940. The method of item 14748, wherein the composition comprises a polymeric carrier, and wherein the polymeric carrier comprises a polymer having hydrophobic domains.

14941. The method of item 14748, wherein the composition comprises a polymeric carrier, and wherein the polymeric carrier comprises a non-conductive polymer.

14942. The method of item 14748, wherein the composition comprises a polymeric carrier, and wherein the polymeric carrier comprises an elastomer.

14943. The method of item 14748, wherein the composition comprises a polymeric carrier, and wherein the polymeric carrier comprises a hydrogel.

14944. The method of item 14748, wherein the composition comprises a polymeric carrier, and wherein the polymeric carrier comprises a silicone polymer.

14945. The method of item 14748, wherein the composition comprises a polymeric carrier, and wherein the polymeric carrier comprises a hydrocarbon polymer.

14946. The method of item 14748, wherein the composition comprises a polymeric carrier, and wherein the polymeric carrier comprises a styrene-derived polymer.

14947. The method of item 14748, wherein the composition comprises a polymeric carrier, and wherein the polymeric carrier comprises a butadiene polymer.

14948. The method of item 14748, wherein the composition comprises a polymeric carrier, and wherein the polymeric carrier comprises a macromer.

14949. The method of item 14748, wherein the composition comprises a polymeric carrier, and wherein the polymeric carrier comprises a poly(ethylene glycol)polymer.

14950. The method of item 14748 wherein the composition comprises a polymeric carrier, and wherein the polymeric carrier comprises an amorphous polymer.

14951. The method of item 14748, wherein the device comprises a lubricious coating.

14952. The method of item 14748 wherein the anti-scarring agent is located within pores or holes of the device.

14953. The method of item 14748 wherein the anti-scarring agent is located within a channel, lumen, or divet of the device.

14954. The method of item 14748, wherein the device comprises a second pharmaceutically active agent.

14955. The method of item 14748 wherein the device comprises an anti-inflammatory agent.

14956. The method of item 14748 wherein the device comprises an agent that inhibits infection.

14957. The method of item 14748 wherein the device comprises an agent that inhibits infection, and wherein the agent is an anthracycline.

14958. The method of item 14748 wherein the device comprises an agent that inhibits infection, and wherein the agent is doxorubicin.

14959. The method of item 14748 wherein the device comprises an agent that inhibits infection, and wherein the agent is mitoxantrone.

14960. The method of item 14748 wherein the device comprises an agent that inhibits infection, and wherein the agent is a fluoropyrimidine.

14961. The method of item 14748 wherein the device comprises an agent that inhibits infection, and wherein the agent is 5-fluorouracil (5-FU).

14962. The method of item 14748 wherein the device comprises an agent that inhibits infection, and wherein the agent is a folic acid antagonist.

14963. The method of item 14748 wherein the device comprises an agent that inhibits infection, and wherein the agent is methotrexate.

14964. The method of item 14748 wherein the device comprises an agent that inhibits infection, and wherein the agent is a podophylotoxin.

14965. The method of item 14748 wherein the device comprises an agent that inhibits infection, and wherein the agent is etoposide.

14966. The method of item 14748 wherein the device comprises an agent that inhibits infection, and wherein the agent is a camptothecin.

14967. The method of item 14748 wherein the device comprises an agent that inhibits infection, and wherein the agent is a hydroxyurea.

14968. The method of item 14748 wherein the device comprises an agent that inhibits infection, and wherein the agent is a platinum complex.

14969. The method of item 14748 wherein the device comprises an agent that inhibits infection, and wherein the agent is cisplatin.

14970. The method of item 14748, further comprising an anti-thrombotic agent.

14971. The method of item 14748 wherein the device comprises a visualization agent.

14972. The method of item 14748 wherein the device comprises a visualization agent, wherein the visualization agent is a radiopaque material, and wherein the radiopaque material comprises a metal, a halogenated compound, or a barium containing compound.

14973. The method of item 14748 wherein the device comprises a visualization agent, wherein the visualization agent is a radiopaque material, and wherein the radiopaque material comprises barium, tantalum, or technetium.

14974. The method of item 14748 wherein the device comprises a visualization agent, and wherein the visualization agent is a MRI responsive material.

14975. The method of item 14748 wherein the device comprises a visualization agent, and wherein the visualization agent comprises a gadolinium chelate.

14976. The method of item 14748 wherein the device comprises a visualization agent, and wherein the visualization agent comprises iron, magnesium, manganese, copper, or chromium.

14977. The method of item 14748 wherein the device comprises a visualization agent, and wherein the visualization agent comprises an iron oxide compound.

14978. The method of item 14748 wherein the device comprises a visualization agent, and wherein the visualization agent comprises a dye, pigment, or colorant.

14979. The method of item 14748 wherein the device comprises an echogenic material.

14980. The method of item 14748 wherein the device comprises an echogenic material, and wherein the echogenic material is in the form of a coating.

14981. The method of item 14748 wherein the device is sterile.

14982. The method of item 14748 wherein the anti-scarring agent is released into tissue in the vicinity of the device after deployment of the device.

14983. The method of item 14748 wherein the anti-scarring agent is released into tissue in the vicinity of the device after deployment of the device, and wherein the tissue is connective tissue.

14984. The method of item 14748 wherein the anti-scarring agent is released into tissue in the vicinity of the device after deployment of the device, and wherein the tissue is muscle tissue.

14985. The method of item 14748 wherein the anti-scarring agent is released into tissue in the vicinity of the device after deployment of the device, and wherein the tissue is nerve tissue.

14986. The method of item 14748 wherein the anti-scarring agent is released into tissue in the vicinity of the device after deployment of the device, and wherein the tissue is epithelium tissue.

14987. The method of item 14748 wherein the anti-scarring agent is released in effective concentrations from the device over a period ranging from the time of deployment of the device to about 1 year.

14988. The method of item 14748 wherein the anti-scarring agent is released in effective concentrations from the device over a period ranging from about 1 month to 6 months.

14989. The method of item 14748 wherein the anti-scarring agent is released in effective concentrations from the device over a period ranging from about 1-90 days.

14990. The method of item 14748 wherein the anti-scarring agent is released in effective concentrations from the device at a constant rate.

14991. The method of item 14748 wherein the anti-scarring agent is released in effective concentrations from the device at an increasing rate.

14992. The method of item 14748 wherein the anti-scarring agent is released in effective concentrations from the device at a decreasing rate.

14993. The method of item 14748 wherein the anti-scarring agent is released in effective concentrations from the composition comprising the anti-scarring agent by diffusion over a period ranging from the time of deployment of the device to about 90 days.

14994. The method of item 14748 wherein the anti-scarring agent is released in effective concentrations from the composition comprising the anti-scarring agent by erosion of the composition over a period ranging from the time of deployment of the device to about 90 days.

14995. The method of item 14748 wherein the device comprises about 0.01 μg to about 10 μg of the anti-scarring agent.

14996. The method of item 14748 wherein the device comprises about 10 μg to about 10 mg of the anti-scarring agent.

14997. The method of item 14748 wherein the device comprises about 10 mg to about 250 mg of the anti-scarring agent.

14998. The method of item 14748 wherein the device comprises about 250 mg to about 1000 mg of the anti-scarring agent.

14999. The method of item 14748 wherein the device comprises about 1000 mg to about 2500 mg of the anti-scarring agent.

15000. The method of item 14748 wherein a surface of the device comprises less than 0.01 μg of the anti-scarring agent per mm² of device surface to which the anti-scarring agent is applied.

15001. The method of item 14748 wherein a surface of the device comprises about 0.01 μg to about 1 μg of the anti-scarring agent per mm² of device surface to which the anti-scarring agent is applied.

15002. The method of item 14748 wherein a surface of the device comprises about 1 μg to about 10 μg of the anti-scarring agent per mm² of device surface to which the anti-scarring agent is applied.

15003. The method of item 14748 wherein a surface of the device comprises about 10 μg to about 250 μg of the anti-scarring agent per mm² of device surface to which the anti-scarring agent is applied.

15004. The method of item 14748 wherein a surface of the device comprises about 250 μg to about 1000 μg of the anti-scarring agent of anti-scarring agent per mm² of device surface to which the anti-scarring agent is applied.

15005. The method of item 14748 wherein a surface of the device comprises about 1000 μg to about 2500 μg of the anti-scarring agent per mm² of device surface to which the anti-scarring agent is applied.

15006. The method of item 14748 wherein the combining is performed by direct affixing the agent or the composition to the implant.

15007. The method of item 14748 wherein the combining is performed by spraying the agent or the component onto the implant.

15008. The method of item 14748 wherein the combining is performed by electrospraying the agent or the composition onto the implant.

15009. The method of item 14748 wherein the combining is performed by dipping the implant into a solution comprising the agent or the composition.

15010. The method of item 14748 wherein the combining is performed by covalently attaching the agent or the composition to the implant.

15011. The method of item 14748 wherein the combining is performed by non-covalently attaching the agent or the composition to the implant.

15012. The method of item 14748 wherein the combining is performed by coating the implant with a substance that contains the agent or the composition.

15013. The method of item 14748 wherein the combining is performed by coating the implant with a substance that absorbs the agent.

15014. The method of item 14748 wherein the combining is performed by interweaving a thread composed of, or coated with, the agent or the composition.

15015. The method of item 14748 wherein the combining is performed by covering all the implant with a sleeve that contains the agent or the composition.

15016. The method of item 14748 wherein the combining is performed by covering a portion of the implant with a sleeve that contains the agent or the composition.

15017. The method of item 14748 wherein the combining is performed by covering all the implant with a cover that contains the agent or the composition.

15018. The method of item 14748 wherein the combining is performed by covering a portion of the implant with a cover that contains the agent or the composition.

15019. The method of item 14748 wherein the combining is performed by covering all the implant with an electrospun fabric that contains the agent or the composition.

15020. The method of item 14748 wherein the combining is performed by covering a portion of the implant with an electrospun fabric that contains the agent or the composition.

15021. The method of item 14748 wherein the combining is performed by covering all the implant with a mesh that contains the agent or the composition.

15022. The method of item 14748 wherein the combining is performed by covering a portion of the implant with a mesh that contains the agent or the composition.

15023. The method of item 14748 wherein the combining is performed by constructing all the implant with the agent or the composition.

15024. The method of item 14748 wherein the combining is performed by constructing a portion of the implant with the agent or the composition.

15025. The method of item 14748 wherein the combining is performed by impregnating the implant with the agent or the composition.

15026. The method of item 14748 wherein the combining is performed by constructing all of the implant from a degradable polymer that releases the agent.

15027. The method of item 14748 wherein the combining is performed by constructing a portion of the implant from a degradable polymer that releases the agent.

15028. The method of item 14748 wherein the combining is performed by dipping the implant into a solution that comprise the agent and an inert solvent for the implant.

15029. The method of item 14748 wherein the combining is performed by dipping the implant into a solution that comprises the agent and a solvent that will swill the implant.

15030. The method of item 14748 wherein the combining is performed by dipping the implant into a solution that comprises the agent and a solvent that will dissolve the implant.

15031. The method of item 14748 wherein the combining is performed by dipping the implant into a solution that comprises the agent, a polymer and an inert solvent for the implant.

15032. The method of item 14748 wherein the combining is performed by dipping the implant into a solution that comprises the agent, a polymer and a solvent that will swill the implant.

15033. The method of item 14748 wherein the combining is performed by dipping the implant into a solution that comprises the agent, a polymer and a solvent that will dissolve the implant.

15034. The method of item 14748 wherein the combining is performed by spraying the implant into a solution that comprises the agent and an inert solvent for the implant.

15035. The method of item 14748 wherein the combining is performed by spraying the implant into a solution that comprises the agent and a solvent that will swill the implant.

15036. The method of item 14748 wherein the combining is performed by spraying the implant into a solution that comprises the agent and a solvent that will dissolve the implant.

15037. The method of item 14748 wherein the combining is performed by spraying the implant into a solution that comprises the agent, a polymer and an inert solvent for the implant.

15038. The method of item 14748 wherein the combining is performed by spraying the implant into a solution that comprises the agent, a polymer and a solvent that will swill the implant.

15039. The method of item 14748 wherein the combining is performed by spraying the implant into a solution that comprises the agent, a polymer and a solvent that will dissolve the implant.

15040. A method of making a medical device comprising: combining a gastrointestinal device (i.e., an implant) and an anti-scarring agent or a composition comprising an anti-scarring agent, wherein the agent inhibits scarring between the device and a host into which the device is implanted.

15041. The method of item 15040 wherein the agent inhibits cell regeneration.

15042. The method of item 15040 wherein the agent inhibits angiogenesis.

15043. The method of item 15040 wherein the agent inhibits fibroblast migration.

15044. The method of item 15040 wherein the agent inhibits fibroblast proliferation.

15045. The method of item 15040 wherein the agent inhibits deposition of extracellular matrix.

15046. The method of item 15040 wherein the agent inhibits tissue remodeling.

15047. The method of item 15040 wherein the agent is an angiogenesis inhibitor.

15048. The method of item 15040 wherein the agent is a 5-lipoxygenase inhibitor or antagonist.

15049. The method of item 15040 wherein the agent is a chemokine receptor antagonist.

15050. The method of item 15040 wherein the agent is a cell cycle inhibitor.

15051. The method of item 15040 wherein the agent is a taxane.

15052. The method of item 15040 wherein the agent is an anti-microtubule agent.

15053. The method of item 15040 wherein the agent is paclitaxel.

15054. The method of item 15040 wherein the agent is not paclitaxel.

15055. The method of item 15040 wherein the agent is an analogue or derivative of paclitaxel.

15056. The method of item 15040 wherein the agent is a vinca alkaloid.

15057. The method of item 15040 wherein the agent is camptothecin or an analogue or derivative thereof.

15058. The method of item 15040 wherein the agent is a podophyllotoxin.

15059. The method of item 15040 wherein the agent is a podophyllotoxin, wherein the podophyllotoxin is etoposide or an analogue or derivative thereof.

15060. The method of item 15040 wherein the agent is an anthracycline.

15061. The method of item 15040 wherein the agent is an anthracycline, wherein the anthracycline is doxorubicin or an analogue or derivative thereof.

15062. The method of item 15040 wherein the agent is an anthracycline, wherein the anthracycline is mitoxantrone or an analogue or derivative thereof.

15063. The method of item 15040 wherein the agent is a platinum compound.

15064. The method of item 15040 wherein the agent is a nitrosourea.

15065. The method of item 15040 wherein the agent is a nitroimidazole.

15066. The method of item 15040 wherein the agent is a folic acid antagonist.

15067. The method of item 15040 wherein the agent is a cytidine analogue.

15068. The method of item 15040 wherein the agent is a pyrimidine analogue.

15069. The method of item 15040 wherein the agent is a fluoropyrimidine analogue.

15070. The method of item 15040 wherein the agent is a purine analogue.

15071. The method of item 15040 wherein the agent is a nitrogen mustard or an analogue or derivative thereof.

15072. The method of item 15040 wherein the agent is a hydroxyurea.

15073. The method of item 15040 wherein the agent is a mytomicin or an analogue or derivative thereof.

15074. The method of item 15040 wherein the agent is an alkyl sulfonate.

15075. The method of item 15040 wherein the agent is a benzamide or an analogue or derivative thereof.

15076. The method of item 15040 wherein the agent is a nicotinamide or an analogue or derivative thereof.

15077. The method of item 15040 wherein the agent is a halogenated sugar or an analogue or derivative thereof.

15078. The method of item 15040 wherein the agent is a DNA alkylating agent.

15079. The method of item 15040 wherein the agent is an anti-microtubule agent.

15080. The method of item 15040 wherein the agent is a topoisomerase inhibitor.

15081. The method of item 15040 wherein the agent is a DNA cleaving agent.

15082. The method of item 15040 wherein the agent is an antimetabolite.

15083. The method of item 15040 wherein the agent inhibits adenosine deaminase.

15084. The method of item 15040 wherein the agent inhibits purine ring synthesis.

15085. The method of item 15040 wherein the agent is a nucleotide interconversion inhibitor.

15086. The method of item 15040 wherein the agent inhibits dihydrofolate reduction.

15087. The method of item 15040 wherein the agent blocks thymidine monophosphate.

15088. The method of item 15040 wherein the agent causes DNA damage.

15089. The method of item 15040 wherein the agent is a DNA intercalation agent.

15090. The method of item 15040 wherein the agent is a RNA synthesis inhibitor.

15091. The method of item 15040 wherein the agent is a pyrimidine synthesis inhibitor.

15092. The method of item 15040 wherein the agent inhibits ribonucleotide synthesis or function.

15093. The method of item 15040 wherein the agent inhibits thymidine monophosphate synthesis or function.

15094. The method of item 15040 wherein the agent inhibits DNA synthesis.

15095. The method of item 15040 wherein the agent causes DNA adduct formation.

15096. The method of item 15040 wherein the agent inhibits protein synthesis.

15097. The method of item 15040 wherein the agent inhibits microtubule function.

15098. The method of item 15040 wherein the agent is a cyclin dependent protein kinase inhibitor.

15099. The method of item 15040 wherein the agent is an epidermal growth factor kinase inhibitor.

15100. The method of item 15040 wherein the agent is an elastase inhibitor.

15101. The method of item 15040 wherein the agent is a factor Xa inhibitor.

15102. The method of item 15040 wherein the agent is a farnesyltransferase inhibitor.

15103. The method of item 15040 wherein the agent is a fibrinogen antagonist.

15104. The method of item 15040 wherein the agent is a guanylate cyclase stimulant.

15105. The method of item 15040 wherein the agent is a heat shock protein 90 antagonist.

15106. The method of item 15040 wherein the agent is a heat shock protein 90 antagonist, wherein the heat shock protein 90 antagonist is geldanamycin or an analogue or derivative thereof.

15107. The method of item 15040 wherein the agent is a guanylate cyclase stimulant.

15108. The method of item 15040 wherein the agent is a HMGCoA reductase inhibitor.

15109. The method of item 15040 wherein the agent is a HMGCoA reductase inhibitor, wherein the HMGCoA reductase inhibitor is simvastatin or an analogue or derivative thereof.

15110. The method of item 15040 wherein the agent is a hydroorotate dehydrogenase inhibitor.

15111. The method of item 15040 wherein the agent is an IKK2 inhibitor.

15112. The method of item 15040 wherein the agent is an IL-1 antagonist.

15113. The method of item 15040 wherein the agent is an ICE antagonist.

15114. The method of item 15040 wherein the agent is an IRAK antagonist.

15115. The method of item 15040 wherein the agent is an IL-4 agonist.

15116. The method of item 15040 wherein the agent is an immunomodulatory agent.

15117. The method of item 15040 wherein the agent is sirolimus or an analogue or derivative thereof.

15118. The method of item 15040 wherein the agent is not sirolimus.

15119. The method of item 15040 wherein the agent is everolimus or an analogue or derivative thereof.

15120. The method of item 15040 wherein the agent is tacrolimus or an analogue or derivative thereof.

15121. The method of item 15040 wherein the agent is not tacrolimus.

15122. The method of item 15040 wherein the agent is biolmus or an analogue or derivative thereof.

15123. The method of item 15040 wherein the agent is tresperimus or an analogue or derivative thereof.

15124. The method of item 15040 wherein the agent is auranofin or an analogue or derivative thereof.

15125. The method of item 15040 wherein the agent is 27-0-demethylrapamycin or an analogue or derivative thereof.

15126. The method of item 15040 wherein the agent is gusperimus or an analogue or derivative thereof.

15127. The method of item 15040 wherein the agent is pimecrolimus or an analogue or derivative thereof.

15128. The method of item 15040 wherein the agent is ABT-578 or an analogue or derivative thereof.

15129. The method of item 15040 wherein the agent is an inosine monophosphate dehydrogenase (IMPDH) inhibitor.

15130. The method of item 15040 wherein the agent is an IMPDH inhibitor, wherein the IMPDH inhibitor is mycophenolic acid or an analogue or derivative thereof.

15131. The method of item 15040 wherein the agent is an IMPDH inhibitor, wherein the IMPDH inhibitor is 1-alpha-25 dihydroxy vitamin D₃ or an analogue or derivative thereof.

15132. The method of item 15040 wherein the agent is a leukotriene inhibitor.

15133. The method of item 15040 wherein the agent is a MCP-1 antagonist.

15134. The method of item 15040 wherein the agent is a MMP inhibitor.

15135. The method of item 15040 wherein the agent is an NF kappa B inhibitor.

15136. The method of item 15040 wherein the agent is an NF kappa B inhibitor, wherein the NF kappa B inhibitor is Bay 11-7082.

15137. The method of item 15040 wherein the agent is an NO agonist.

15138. The method of item 15040 wherein the agent is a p38 MAP kinase inhibitor.

15139. The method of item 15040 wherein the agent is a p38 MAP kinase inhibitor, wherein the p38 MAP kinase inhibitor is SB 202190.

15140. The method of item 15040 wherein the agent is a phosphodiesterase inhibitor.

15141. The method of item 15040 wherein the agent is a TGF beta inhibitor.

15142. The method of item 15040 wherein the agent is a thromboxane A2 antagonist.

15143. The method of item 15040 wherein the agent is a TNFa antagonist.

15144. The method of item 15040 wherein the agent is a TACE inhibitor.

15145. The method of item 15040 wherein the agent is a tyrosine kinase inhibitor.

15146. The method of item 15040 wherein the agent is a vitronectin inhibitor.

15147. The method of item 15040 wherein the agent is a fibroblast growth factor inhibitor.

15148. The method of item 15040 wherein the agent is a protein kinase inhibitor.

15149. The method of item 15040 wherein the agent is a PDGF receptor kinase inhibitor.

15150. The method of item 15040 wherein the agent is an endothelial growth factor receptor kinase inhibitor.

15151. The method of item 15040 wherein the agent is a retinoic acid receptor antagonist.

15152. The method of item 15040 wherein the agent is a platelet derived growth factor receptor kinase inhibitor.

15153. The method of item 15040 wherein the agent is a fibronogin antagonist.

15154. The method of item 15040 wherein the agent is an antimycotic agent.

15155. The method of item 15040 wherein the agent is an antimycotic agent, wherein the antimycotic agent is sulconizole.

15156. The method of item 15040 wherein the agent is a bisphosphonate.

15157. The method of item 15040 wherein the agent is a phospholipase A1 inhibitor.

15158. The method of item 15040 wherein the agent is a histamine H1/H2/H3 receptor antagonist.

15159. The method of item 15040 wherein the agent is a macrolide antibiotic.

15160. The method of item 15040 wherein the agent is a GPIIb/IIIa receptor antagonist.

15161. The method of item 15040 wherein the agent is an endothelin receptor antagonist.

15162. The method of item 15040 wherein the agent is a peroxisome proliferator-activated receptor agonist.

15163. The method of item 15040 wherein the agent is an estrogen receptor agent.

15164. The method of item 15040 wherein the agent is a somastostatin analogue.

15165. The method of item 15040 wherein the agent is a neurokinin 1 antagonist.

15166. The method of item 15040 wherein the agent is a neurokinin 3 antagonist.

15167. The method of item 15040 wherein the agent is a VLA-4 antagonist.

15168. The method of item 15040 wherein the agent is an osteoclast inhibitor.

15169. The method of item 15040 wherein the agent is a DNA topoisomerase ATP hydrolyzing inhibitor.

15170. The method of item 15040 wherein the agent is an angiotensin I converting enzyme inhibitor.

15171. The method of item 15040 wherein the agent is an angiotensin II antagonist.

15172. The method of item 15040 wherein the agent is an enkephalinase inhibitor.

15173. The method of item 15040 wherein the agent is a peroxisome proliferator-activated receptor gamma agonist insulin sensitizer.

15174. The method of item 15040 wherein the agent is a protein kinase C inhibitor.

15175. The method of item 15040 wherein the agent is a ROCK (rho-associated kinase) inhibitor.

15176. The method of item 15040 wherein the agent is a CXCR3 inhibitor.

15177. The method of item 15040 wherein the agent is an Itk inhibitor.

15178. The method of item 15040 wherein the agent is a cytosolic phospholipase A₂-alpha inhibitor.

15179. The method of item 15040 wherein the agent is a PPAR agonist.

15180. The method of item 15040 wherein the agent is an immunosuppressant.

15181. The method of item 15040 wherein the agent is an Erb inhibitor.

15182. The method of item 15040 wherein the agent is an apoptosis agonist.

15183. The method of item 15040 wherein the agent is a lipocortin agonist.

15184. The method of item 15040 wherein the agent is a VCAM-1 antagonist.

15185. The method of item 15040 wherein the agent is a collagen antagonist.

15186. The method of item 15040 wherein the agent is an alpha 2 integrin antagonist.

15187. The method of item 15040 wherein the agent is a TNF alpha inhibitor.

15188. The method of item 15040 wherein the agent is a nitric oxide inhibitor.

15189. The method of item 15040 wherein the agent is a cathepsin inhibitor.

15190. The method of item 15040 wherein the agent is not an anti-inflammatory agent.

15191. The method of item 15040 wherein the agent is not a steroid.

15192. The method of item 15040 wherein the agent is not a glucocorticosteroid.

15193. The method of item 15040 wherein the agent is not dexamethasone.

15194. The method of item 15040 wherein the agent is not an anti-infective agent.

15195. The method of item 15040 wherein the agent is not an antibiotic.

15196. The method of item 15040 wherein the agent is not an anti-fungal agent.

15197. The method of item 15040, wherein the composition comprises a polymer.

15198. The method of item 15040, wherein the composition comprises a polymeric carrier.

15199. The method of item 15040 wherein the anti-scarring agent inhibits adhesion between the device and a host into which the device is implanted.

15200. The method of item 15040 wherein the device delivers the anti-scarring agent locally to tissue proximate to the device.

15201. The method of item 15040 wherein the device has a coating that comprises the anti-scarring agent.

15202. The method of item 15040, wherein the device has a coating that comprises the agent and is disposed on a surface of the implant.

15203. The method of item 15040, wherein the device has a coating that comprises the agent and directly contacts the implant.

15204. The method of item 15040, wherein the device has a coating that comprises the agent and indirectly contacts the implant.

15205. The method of item 15040, wherein the device has a coating that comprises the agent and partially covers the implant.

15206. The method of item 15040, wherein the device has a coating that comprises the agent and completely covers the implant.

15207. The method of item 15040, wherein the device has a uniform coating.

15208. The method of item 15040, wherein the device has a non-uniform coating.

15209. The method of item 15040, wherein the device has a discontinuous coating.

15210. The method of item 15040, wherein the device has a patterned coating.

15211. The method of item 15040, wherein the device has a coating with a thickness of 100 μm or less.

15212. The method of item 15040, wherein the device has a coating with a thickness of 10 μm or less.

15213. The method of item 15040, wherein the device has a coating, and the coating adheres to the surface of the implant upon deployment of the implant.

15214. The method of item 15040, wherein the device has a coating, and wherein the coating is stable at room temperature for a period of 1 year.

15215. The method of item 15040, wherein the device has a coating, and wherein the anti-scarring agent is present in the coating in an amount ranging between about 0.0001% to about 1% by weight.

15216. The method of item 15040, wherein the device has a coating, and wherein the anti-scarring agent is present in the coating in an amount ranging between about 1% to about 10% by weight.

15217. The method of item 15040, wherein the device has a coating, and wherein the anti-scarring agent is present in the coating in an amount ranging between about 10% to about 25% by weight.

15218. The method of item 15040, wherein the device has a coating, and wherein the anti-scarring agent is present in the coating in an amount ranging between about 25% to about 70% by weight.

15219. The method of item 15040, wherein the device has a coating, and wherein the coating further comprises a polymer.

15220. The method of item 15040, wherein the device has a first coating having a first composition and a second coating having a second composition.

15221. The method of item 15040, wherein the device has a first coating having a first composition and a second coating having a second composition, wherein the first composition and the second composition are different.

15222. The method of item 15040, wherein the composition comprises a polymer.

15223. The method of item 15040, wherein the composition comprises a polymeric carrier.

15224. The method of item 15040, wherein the composition comprises a polymeric carrier, and wherein the polymeric carrier comprises a copolymer.

15225. The method of item 15040, wherein the composition comprises a polymeric carrier, and wherein the polymeric carrier comprises a block copolymer.

15226. The method of item 15040, wherein the composition comprises a polymeric carrier, and wherein the polymeric carrier comprises a random copolymer.

15227. The method of item 15040, wherein the composition comprises a polymeric carrier, and wherein the polymeric carrier comprises a biodegradable polymer.

15228. The method of item 15040, wherein the composition comprises a polymeric carrier, and wherein the polymeric carrier comprises a non-biodegradable polymer.

15229. The method of item 15040, wherein the composition comprises a polymeric carrier, and wherein the polymeric carrier comprises a hydrophilic polymer.

15230. The method of item 15040, wherein the composition comprises a polymeric carrier, and wherein the polymeric carrier comprises a hydrophobic polymer.

15231. The method of item 15040, wherein the composition comprises a polymeric carrier, and wherein the polymeric carrier comprises a polymer having hydrophilic domains.

15232. The method of item 15040, wherein the composition comprises a polymeric carrier, and wherein the polymeric carrier comprises a polymer having hydrophobic domains.

15233. The method of item 15040, wherein the composition comprises a polymeric carrier, and wherein the polymeric carrier comprises a non-conductive polymer.

15234. The method of item 15040, wherein the composition comprises a polymeric carrier, and wherein the polymeric carrier comprises an elastomer.

15235. The method of item 15040, wherein the composition comprises a polymeric carrier, and wherein the polymeric carrier comprises a hydrogel.

1.5236. The method of item 15040, wherein the composition comprises a polymeric carrier, and wherein the polymeric carrier comprises a silicone polymer.

15237. The method of item 15040, wherein the composition comprises a polymeric carrier, and wherein the polymeric carrier comprises a hydrocarbon polymer.

15238. The method of item 15040, wherein the composition comprises a polymeric carrier, and wherein the polymeric carrier comprises a styrene-derived polymer.

15239. The method of item 15040, wherein the composition comprises a polymeric carrier, and wherein the polymeric carrier comprises a butadiene polymer.

15240. The method of item 15040, wherein the composition comprises a polymeric carrier, and wherein the polymeric carrier comprises a macromer.

15241. The method of item 15040, wherein the composition comprises a polymeric carrier, and wherein the polymeric carrier comprises a poly(ethylene glycol)polymer.

15242. The method of item 15040 wherein the composition comprises a polymeric carrier, and wherein the polymeric carrier comprises an amorphous polymer.

15243. The method of item 15040, wherein the device comprises a lubricious coating.

15244. The method of item 15040 wherein the anti-scarring agent is located within pores or holes of the device.

15245. The method of item 15040 wherein the anti-scarring agent is located within a channel, lumen, or divet of the device.

15246. The method of item 15040, wherein the device comprises a second pharmaceutically active agent.

15247. The method of item 15040 wherein the device comprises an anti-inflammatory agent.

15248. The method of item 15040 wherein the device comprises an agent that inhibits infection.

15249. The method of item 15040 wherein the device comprises an agent that inhibits infection, and wherein the agent is an anthracycline.

15250. The method of item 15040 wherein the device comprises an agent that inhibits infection, and wherein the agent is doxorubicin.

15251. The method of item 15040 wherein the device comprises an agent that inhibits infection, and wherein the agent is mitoxantrone.

15252. The method of item 15040 wherein the device comprises an agent that inhibits infection, and wherein the agent is a fluoropyrimidine.

15253. The method of item 15040 wherein the device comprises an agent that inhibits infection, and wherein the agent is 5-fluorouracil (5-FU).

15254. The method of item 15040 wherein the device comprises an agent that inhibits infection, and wherein the agent is a folic acid antagonist.

15255. The method of item 15040 wherein the device comprises an agent that inhibits infection, and wherein the agent is methotrexate.

15256. The method of item 15040 wherein the device comprises an agent that inhibits infection, and wherein the agent is a podophylotoxin.

15257. The method of item 15040 wherein the device comprises an agent that inhibits infection, and wherein the agent is etoposide.

15258. The method of item 15040 wherein the device comprises an agent that inhibits infection, and wherein the agent is a camptothecin.

15259. The method of item 15040 wherein the device comprises an agent that inhibits infection, and wherein the agent is a hydroxyurea.

15260. The method of item 15040 wherein the device comprises an agent that inhibits infection, and wherein the agent is a platinum complex.

15261. The method of item 15040 wherein the device comprises an agent that inhibits infection, and wherein the agent is cisplatin.

15262. The method of item 15040, further comprising an anti-thrombotic agent.

15263. The method of item 15040 wherein the device comprises a visualization agent.

15264. The method of item 15040 wherein the device comprises a visualization agent, wherein the visualization agent is a radiopaque material, and wherein the radiopaque material comprises a metal, a halogenated compound, or a barium containing compound.

15265. The method of item 15040 wherein the device comprises a visualization agent, wherein the visualization agent is a radiopaque material, and wherein the radiopaque material comprises barium, tantalum, or technetium.

15266. The method of item 15040 wherein the device comprises a visualization agent, and wherein the visualization agent is a MRI responsive material.

15267. The method of item 15040 wherein the device comprises a visualization agent, and wherein the visualization agent comprises a gadolinium chelate.

15268. The method of item 15040 wherein the device comprises a visualization agent, and wherein the visualization agent comprises iron, magnesium, manganese, copper, or chromium.

15269. The method of item 15040 wherein the device comprises a visualization agent, and wherein the visualization agent comprises an iron oxide compound.

15270. The method of item 15040 wherein the device comprises a visualization agent, and wherein the visualization agent comprises a dye, pigment, or colorant.

15271. The method of item 15040 wherein the device comprises an echogenic material.

15272. The method of item 15040 wherein the device comprises an echogenic material, and wherein the echogenic material is in the form of a coating.

15273. The method of item 15040 wherein the device is sterile.

15274. The method of item 15040 wherein the anti-scarring agent is released into tissue in the vicinity of the device after deployment of the device.

15275. The method of item 15040 wherein the anti-scarring agent is released into tissue in the vicinity of the device after deployment of the device, and wherein the tissue is connective tissue.

15276. The method of item 15040 wherein the anti-scarring agent is released into tissue in the vicinity of the device after deployment of the device, and wherein the tissue is muscle tissue.

15277. The method of item 15040 wherein the anti-scarring agent is released into tissue in the vicinity of the device after deployment of the device, and wherein the tissue is nerve tissue.

15278. The method of item 15040 wherein the anti-scarring agent is released into tissue in the vicinity of the device after deployment of the device, and wherein the tissue is epithelium tissue.

15279. The method of item 15040 wherein the anti-scarring agent is released in effective concentrations from the device over a period ranging from the time of deployment of the device to about 1 year.

15280. The method of item 15040 wherein the anti-scarring agent is released in effective concentrations from the device over a period ranging from about 1 month to 6 months.

15281. The method of item 15040 wherein the anti-scarring agent is released in effective concentrations from the device over a period ranging from about 1-90 days.

15282. The method of item 15040 wherein the anti-scarring agent is released in effective concentrations from the device at a constant rate.

15283. The method of item 15040 wherein the anti-scarring agent is released in effective concentrations from the device at an increasing rate.

15284. The method of item 15040 wherein the anti-scarring agent is released in effective concentrations from the device at a decreasing rate.

15285. The method of item 15040 wherein the anti-scarring agent is released in effective concentrations from the composition comprising the anti-scarring agent by diffusion over a period ranging from the time of deployment of the device to about 90 days.

15286. The method of item 15040 wherein the anti-scarring agent is released in effective concentrations from the composition comprising the anti-scarring agent by erosion of the composition over a period ranging from the time of deployment of the device to about 90 days.

15287. The method of item 15040 wherein the device comprises about 0.01 μg to about 10 μg of the anti-scarring agent.

15288. The method of item 15040 wherein the device comprises about 10 μg to about 10 mg of the anti-scarring agent.

15289. The method of item 15040 wherein the device comprises about 10 mg to about 250 mg of the anti-scarring agent.

15290. The method of item 15040 wherein the device comprises about 250 mg to about 1000 mg of the anti-scarring agent.

15291. The method of item 15040 wherein the device comprises about 1000 mg to about 2500 mg of the anti-scarring agent.

15292. The method of item 15040 wherein a surface of the device comprises less than 0.01 μg of the anti-scarring agent per mm² of device surface to which the anti-scarring agent is applied.

15293. The method of item 15040 wherein a surface of the device comprises about 0.01 μg to about 1 μg of the anti-scarring agent per mm² of device surface to which the anti-scarring agent is applied.

15294. The method of item 15040 wherein a surface of the device comprises about 1 μg to about 10 μg of the anti-scarring agent per mm² of device surface to which the anti-scarring agent is applied.

15295. The method of item 15040 wherein a surface of the device comprises about 10 μg to about 250 μg of the anti-scarring agent per mm² of device surface to which the anti-scarring agent is applied.

15296. The method of item 15040 wherein a surface of the device comprises about 250 μg to about 1000 μg of the anti-scarring agent of anti-scarring agent per mm² of device surface to which the anti-scarring agent is applied.

15297. The method of item 15040 wherein a surface of the device comprises about 1000 μg to about 2500 μg of the anti-scarring agent per mm² of device surface to which the anti-scarring agent is applied.

15298. The method of item 15040 wherein the combining is performed by direct affixing the agent or the composition to the implant.

15299. The method of item 15040 wherein the combining is performed by spraying the agent or the component onto the implant.

15300. The method of item 15040 wherein the combining is performed by electrospraying the agent or the composition onto the implant.

15301. The method of item 15040 wherein the combining is performed by dipping the implant into a solution comprising the agent or the composition.

15302. The method of item 15040 wherein the combining is performed by covalently attaching the agent or the composition to the implant.

15303. The method of item 15040 wherein the combining is performed by non-covalently attaching the agent or the composition to the implant.

15304. The method of item 15040 wherein the combining is performed by coating the implant with a substance that contains the agent or the composition.

15305. The method of item 15040 wherein the combining is performed by coating the implant with a substance that absorbs the agent.

15306. The method of item 15040 wherein the combining is performed by interweaving a thread composed of, or coated with, the agent or the composition.

15307. The method of item 15040 wherein the combining is performed by covering all the implant with a sleeve that contains the agent or the composition.

15308. The method of item 15040 wherein the combining is performed by covering a portion of the implant with a sleeve that contains the agent or the composition.

15309. The method of item 15040 wherein the combining is performed by covering all the implant with a cover that contains the agent or the composition.

15310. The method of item 15040 wherein the combining is performed by covering a portion of the implant with a cover that contains the agent or the composition.

15311. The method of item 15040 wherein the combining is performed by covering all the implant with an electrospun fabric that contains the agent or the composition.

15312. The method of item 15040 wherein the combining is performed by covering a portion of the implant with an electrospun fabric that contains the agent or the composition.

15313. The method of item 15040 wherein the combining is performed by covering all the implant with a mesh that contains the agent or the composition.

15314. The method of item 15040 wherein the combining is performed by covering a portion of the implant with a mesh that contains the agent or the composition.

15315. The method of item 15040 wherein the combining is performed by constructing all the implant with the agent or the composition.

15316. The method of item 15040 wherein the combining is performed by constructing a portion of the implant with the agent or the composition.

15317. The method of item 15040 wherein the combining is performed by impregnating the implant with the agent or the composition.

15318. The method of item 15040 wherein the combining is performed by constructing all of the implant from a degradable polymer that releases the agent.

15319. The method of item 15040 wherein the combining is performed by constructing a portion of the implant from a degradable polymer that releases the agent.

15320. The method of item 15040 wherein the combining is performed by dipping the implant into a solution that comprise the agent and an inert solvent for the implant.

15321. The method of item 15040 wherein the combining is performed by dipping the implant into a solution that comprises the agent and a solvent that will swill the implant.

15322. The method of item 15040 wherein the combining is performed by dipping the implant into a solution that comprises the agent and a solvent that will dissolve the implant.

15323. The method of item 15040 wherein the combining is performed by dipping the implant into a solution that comprises the agent, a polymer and an inert solvent for the implant.

15324. The method of item 15040 wherein the combining is performed by dipping the implant into a solution that comprises the agent, a polymer and a solvent that will swill the implant.

15325. The method of item 15040 wherein the combining is performed by dipping the implant into a solution that comprises the agent, a polymer and a solvent that will dissolve the implant.

15326. The method of item 15040 wherein the combining is performed by spraying the implant into a solution that comprises the agent and an inert solvent for the implant.

15327. The method of item 15040 wherein the combining is performed by spraying the implant into a solution that comprises the agent and a solvent that will swill the implant.

15328. The method of item 15040 wherein the combining is performed by spraying the implant into a solution that comprises the agent and a solvent that will dissolve the implant.

15329. The method of item 15040 wherein the combining is performed by spraying the implant into a solution that comprises the agent, a polymer and an inert solvent for the implant.

15330. The method of item 15040 wherein the combining is performed by spraying the implant into a solution that comprises the agent, a polymer and a solvent that will swill the implant.

15331. The method of item 15040 wherein the combining is performed by spraying the implant into a solution that comprises the agent, a polymer and a solvent that will dissolve the implant.

15332. The method of item 15040 wherein the implant is a GI tube for drainage.

15333. The method of item 15040 wherein the implant is a GI tube for feeding.

15334. The method of item 15040 wherein the implant is a portosystemic shunt.

15335. The method of item 15040 wherein the implant is a shunt for ascites.

15336. The method of item 15040 wherein the implant is a nasogastric tube.

15337. The method of item 15040 wherein the implant is a nosoenteral tube.

15338. The method of item 15040 wherein the implant is a gastrostomy feeding tube.

15339. The method of item 15040 wherein the implant is a percutaneous feeding tube.

15340. The method of item 15040 wherein the implant is a colostomy device.

15341. The method of item 15040 wherein the implant is a biliary T-tube.

15342. The method of item 15040 wherein the implant is a biliary stone removal device.

15343. The method of item 15040 wherein the implant is a dilation balloon.

15344. The method of item 15040 wherein the implant is a dilation catheter.

15345. The method of item 15040 wherein the implant is an enteral feeding device.

15346. The method of item 15040 wherein the implant is an esophageal stent.

15347. The method of item 15040 wherein the implant is a biliary stent.

15348. The method of item 15040 wherein the implant is a pancreatic stent.

15349. A method of making a medical device comprising: combining a spinal implant and an anti-scarring agent or a composition comprising an anti-scarring agent, wherein the agent inhibits scarring between the device and a host into which the device is implanted.

15350. The method of item 15349 wherein the agent inhibits cell regeneration.

15351. The method of item 15349 wherein the agent inhibits angiogenesis.

15352. The method of item 15349 wherein the agent inhibits fibroblast migration.

15353. The method of item 15349 wherein the agent inhibits fibroblast proliferation.

15354. The method of item 15349 wherein the agent inhibits deposition of extracellular matrix.

15355. The method of item 15349 wherein the agent inhibits tissue remodeling.

15356. The method of item 15349 wherein the agent is an angiogenesis inhibitor.

15357. The method of item 15349 wherein the agent is a 5-lipoxygenase inhibitor or antagonist.

15358. The method of item 15349 wherein the agent is a chemokine receptor antagonist.

15359. The method of item 15349 wherein the agent is a cell cycle inhibitor.

15360. The method of item 15349 wherein the agent is a taxane.

15361. The method of item 15349 wherein the agent is an anti-microtubule agent.

15362. The method of item 15349 wherein the agent is paclitaxel.

15363. The method of item 15349 wherein the agent is not paclitaxel.

15364. The method of item 15349 wherein the agent is an analogue or derivative of paclitaxel.

15365. The method of item 15349 wherein the agent is a vinca alkaloid.

15366. The method of item 15349 wherein the agent is camptothecin or an analogue or derivative thereof.

15367. The method of item 15349 wherein the agent is a podophyllotoxin.

15368. The method of item 15349 wherein the agent is a podophyllotoxin, wherein the podophyllotoxin is etoposide or an analogue or derivative thereof.

15369. The method of item 15349 wherein the agent is an anthracycline.

15370. The method of item 15349 wherein the agent is an anthracycline, wherein the anthracycline is doxorubicin or an analogue or derivative thereof.

15371. The method of item 15349 wherein the agent is an anthracycline, wherein the anthracycline is mitoxantrone or an analogue or derivative thereof.

15372. The method of item 15349 wherein the agent is a platinum compound.

15373. The method of item 15349 wherein the agent is a nitrosourea.

15374. The method of item 15349 wherein the agent is a nitroimidazole.

15375. The method of item 15349 wherein the agent is a folic acid antagonist.

15376. The method of item 15349 wherein the agent is a cytidine analogue.

15377. The method of item 15349 wherein the agent is a pyrimidine analogue.

15378. The method of item 15349 wherein the agent is a fluoropyrimidine analogue.

15379. The method of item 15349 wherein the agent is a purine analogue.

15380. The method of item 15349 wherein the agent is a nitrogen mustard or an analogue or derivative thereof.

15381. The method of item 15349 wherein the agent is a hydroxyurea.

15382. The method of item 15349 wherein the agent is a mytomicin or an analogue or derivative thereof.

15383. The method of item 15349 wherein the agent is an alkyl sulfonate.

15384. The method of item 15349 wherein the agent is a benzamide or an analogue or derivative thereof.

15385. The method of item 15349 wherein the agent is a nicotinamide or an analogue or derivative thereof.

15386. The method of item 15349 wherein the agent is a halogenated sugar or an analogue or derivative thereof.

15387. The method of item 15349 wherein the agent is a DNA alkylating agent.

15388. The method of item 15349 wherein the agent is an anti-microtubule agent.

15389. The method of item 15349 wherein the agent is a topoisomerase inhibitor.

15390. The method of item 15349 wherein the agent is a DNA cleaving agent.

15391. The method of item 15349 wherein the agent is an antimetabolite.

15392. The method of item 15349 wherein the agent inhibits adenosine deaminase.

15393. The method of item 15349 wherein the agent inhibits purine ring synthesis.

15394. The method of item 15349 wherein the agent is a nucleotide interconversion inhibitor.

15395. The method of item 15349 wherein the agent inhibits dihydrofolate reduction.

15396. The method of item 15349 wherein the agent blocks thymidine monophosphate.

15397. The method of item 15349 wherein the agent causes DNA damage.

15398. The method of item 15349 wherein the agent is a DNA intercalation agent.

15399. The method of item 15349 wherein the agent is a RNA synthesis inhibitor.

15400. The method of item 15349 wherein the agent is a pyrimidine synthesis inhibitor.

15401. The method of item 15349 wherein the agent inhibits ribonucleotide synthesis or function.

15402. The method of item 15349 wherein the agent inhibits thymidine monophosphate synthesis or function.

15403. The method of item 15349 wherein the agent inhibits DNA synthesis.

15404. The method of item 15349 wherein the agent causes DNA adduct formation.

15405. The method of item 15349 wherein the agent inhibits protein synthesis.

15406. The method of item 15349 wherein the agent inhibits microtubule function.

15407. The method of item 15349 wherein the agent is a cyclin dependent protein kinase inhibitor.

15408. The method of item 15349 wherein the agent is an epidermal growth factor kinase inhibitor.

15409. The method of item 15349 wherein the agent is an elastase inhibitor.

15410. The method of item 15349 wherein the agent is a factor Xa inhibitor.

15411. The method of item 15349 wherein the agent is a farnesyltransferase inhibitor.

15412. The method of item 15349 wherein the agent is a fibrinogen antagonist.

15413. The method of item 15349 wherein the agent is a guanylate cyclase stimulant.

15414. The method of item 15349 wherein the agent is a heat shock protein 90 antagonist.

15415. The method of item 15349 wherein the agent is a heat shock protein 90 antagonist, wherein the heat shock protein 90 antagonist is geldanamycin or an analogue or derivative thereof.

15416. The method of item 15349 wherein the agent is a guanylate cyclase stimulant.

15417. The method of item 15349 wherein the agent is a HMGCoA reductase inhibitor.

15418. The method of item 15349 wherein the agent is a HMGCoA reductase inhibitor, wherein the HMGCoA reductase inhibitor is simvastatin or an analogue or derivative thereof.

15419. The method of item 15349 wherein the agent is a hydroorotate dehydrogenase inhibitor.

15420. The method of item 15349 wherein the agent is an IKK2 inhibitor.

15421. The method of item 15349 wherein the agent is an IL-1 antagonist.

15422. The method of item 15349 wherein the agent is an ICE antagonist.

15423. The method of item 15349 wherein the agent is an IRAK antagonist.

15424. The method of item 15349 wherein the agent is an IL-4 agonist.

15425. The method of item 15349 wherein the agent is an immunomodulatory agent.

15426. The method of item 15349 wherein the agent is sirolimus or an analogue or derivative thereof.

15427. The method of item 15349 wherein the agent is not sirolimus.

15428. The method of item 15349 wherein the agent is everolimus or an analogue or derivative thereof.

15429. The method of item 15349 wherein the agent is tacrolimus or an analogue or derivative thereof.

15430. The method of item 15349 wherein the agent is not tacrolimus.

15431. The method of item 15349 wherein the agent is biolmus or an analogue or derivative thereof.

15432. The method of item 15349 wherein the agent is tresperimus or an analogue or derivative thereof.

15433. The method of item 15349 wherein the agent is auranofin or an analogue or derivative thereof.

15434. The method of item 15349 wherein the agent is 27-0-demethylrapamycin or an analogue or derivative thereof.

15435. The method of item 15349 wherein the agent is gusperimus or an analogue or derivative thereof.

15436. The method of item 15349 wherein the agent is pimecrolimus or an analogue or derivative thereof.

15437. The method of item 15349 wherein the agent is ABT-578 or an analogue or derivative thereof.

15438. The method of item 15349 wherein the agent is an inosine monophosphate dehydrogenase (IMPDH) inhibitor.

15439. The method of item 15349 wherein the agent is an IMPDH inhibitor, wherein the IMPDH inhibitor is mycophenolic acid or an analogue or derivative thereof.

15440. The method of item 15349 wherein the agent is an IMPDH inhibitor, wherein the IMPDH inhibitor is 1-alpha-25 dihydroxy vitamin D₃ or an analogue or derivative thereof.

15441. The method of item 15349 wherein the agent is a leukotriene inhibitor.

15442. The method of item 15349 wherein the agent is a MCP-1 antagonist.

15443. The method of item 15349 wherein the agent is a MMP inhibitor.

15444. The method of item 15349 wherein the agent is an NF kappa B inhibitor.

15445. The method of item 15349 wherein the agent is an NF kappa B inhibitor, wherein the NF kappa B inhibitor is Bay 11-7082.

15446. The method of item 15349 wherein the agent is an NO agonist.

15447. The method of item 15349 wherein the agent is a p38 MAP kinase inhibitor.

15448. The method of item 15349 wherein the agent is a p38 MAP kinase inhibitor, wherein the p38 MAP kinase inhibitor is SB 202190.

15449. The method of item 15349 wherein the agent is a phosphodiesterase inhibitor.

15450. The method of item 15349 wherein the agent is a TGF beta inhibitor.

15451. The method of item 15349 wherein the agent is a thromboxane A2 antagonist.

15452. The method of item 15349 wherein the agent is a TNFa antagonist.

15453. The method of item 15349 wherein the agent is a TACE inhibitor.

15454. The method of item 15349 wherein the agent is a tyrosine kinase inhibitor.

15455. The method of item 15349 wherein the agent is a vitronectin inhibitor.

15456. The method of item 15349 wherein the agent is a fibroblast growth factor inhibitor.

15457. The method of item 15349 wherein the agent is a protein kinase inhibitor.

15458. The method of item 15349 wherein the agent is a PDGF receptor kinase inhibitor.

15459. The method of item 15349 wherein the agent is an endothelial growth factor receptor kinase inhibitor.

15460. The method of item 15349 wherein the agent is a retinoic acid receptor antagonist.

15461. The method of item 15349 wherein the agent is a platelet derived growth factor receptor kinase inhibitor.

15462. The method of item 15349 wherein the agent is a fibronogin antagonist.

15463. The method of item 15349 wherein the agent is an antimycotic agent.

15464. The method of item 15349 wherein the agent is an antimycotic agent, wherein the antimycotic agent is sulconizole.

15465. The method of item 15349 wherein the agent is a bisphosphonate.

15466. The method of item 15349 wherein the agent is a phospholipase A1 inhibitor.

15467. The method of item 15349 wherein the agent is a histamine H1/H2/H3 receptor antagonist.

15468. The method of item 15349 wherein the agent is a macrolide antibiotic.

15469. The method of item 15349 wherein the agent is a GPIIb/IIIa receptor antagonist.

15470. The method of item 15349 wherein the agent is an endothelin receptor antagonist.

15471. The method of item 15349 wherein the agent is a peroxisome proliferator-activated receptor agonist.

15472. The method of item 15349 wherein the agent is an estrogen receptor agent.

15473. The method of item 15349 wherein the agent is a somastostatin analogue.

15474. The method of item 15349 wherein the agent is a neurokinin 1 antagonist.

15475. The method of item 15349 wherein the agent is a neurokinin 3 antagonist.

15476. The method of item 15349 wherein the agent is a VLA-4 antagonist.

15477. The method of item 15349 wherein the agent is an osteoclast inhibitor.

15478. The method of item 15349 wherein the agent is a DNA topoisomerase ATP hydrolyzing inhibitor.

15479. The method of item 15349 wherein the agent is an angiotensin I converting enzyme inhibitor.

15480. The method of item 15349 wherein the agent is an angiotensin II antagonist.

15481. The method of item 15349 wherein the agent is an enkephalinase inhibitor.

15482. The method of item 15349 wherein the agent is a peroxisome proliferator-activated receptor gamma agonist insulin sensitizer.

15483. The method of item 15349 wherein the agent is a protein kinase C inhibitor.

15484. The method of item 15349 wherein the agent is a ROCK (rho-associated kinase) inhibitor.

15485. The method of item 15349 wherein the agent is a CXCR3 inhibitor.

15486. The method of item 15349 wherein the agent is an Itk inhibitor.

15487. The method of item 15349 wherein the agent is a cytosolic phospholipase A₂-alpha inhibitor.

15488. The method of item 15349 wherein the agent is a PPAR agonist.

15489. The method of item 15349 wherein the agent is an immunosuppressant.

15490. The method of item 15349 wherein the agent is an Erb inhibitor.

15491. The method of item 15349 wherein the agent is an apoptosis agonist.

15492. The method of item 15349 wherein the agent is a lipocortin agonist.

15493. The method of item 15349 wherein the agent is a VCAM-1 antagonist.

15494. The method of item 15349 wherein the agent is a collagen antagonist.

15495. The method of item 15349 wherein the agent is an alpha 2 integrin antagonist.

15496. The method of item 15349 wherein the agent is a TNF alpha inhibitor.

15497. The method of item 15349 wherein the agent is a nitric oxide inhibitor.

15498. The method of item 15349 wherein the agent is a cathepsin inhibitor.

15499. The method of item 15349 wherein the agent is not an anti-inflammatory agent.

15500. The method of item 15349 wherein the agent is not a steroid.

15501. The method of item 15349 wherein the agent is not a glucocorticosteroid.

15502. The method of item 15349 wherein the agent is not dexamethasone.

15503. The method of item 15349 wherein the agent is not an anti-infective agent.

15504. The method of item 15349 wherein the agent is not an antibiotic.

15505. The method of item 15349 wherein the agent is not an anti-fungal agent.

15506. The method of item 15349, wherein the composition comprises a polymer.

15507. The method of item 15349, wherein the composition comprises a polymeric carrier.

15508. The method of item 15349 wherein the anti-scarring agent inhibits adhesion between the device and a host into which the device is implanted.

15509. The method of item 15349 wherein the device delivers the anti-scarring agent locally to tissue proximate to the device.

15510. The method of item 15349 wherein the device has a coating that comprises the anti-scarring agent.

15511. The method of item 15349, wherein the device has a coating that comprises the agent and is disposed on a surface of the implant.

15512. The method of item 15349, wherein the device has a coating that comprises the agent and directly contacts the implant.

15513. The method of item 15349, wherein the device has a coating that comprises the agent and indirectly contacts the implant.

15514. The method of item 15349, wherein the device has a coating that comprises the agent and partially covers the implant.

15515. The method of item 15349, wherein the device has a coating that comprises the agent and completely covers the implant.

15516. The method of item 15349, wherein the device has a uniform coating.

15517. The method of item 15349, wherein the device has a non-uniform coating.

15518. The method of item 15349, wherein the device has a discontinuous coating.

15519. The method of item 15349, wherein the device has a patterned coating.

15520. The method of item 15349, wherein the device has a coating with a thickness of 100 μm or less.

15521. The method of item 15349, wherein the device has a coating with a thickness of 10 μm or less.

15522. The method of item 15349, wherein the device has a coating, and the coating adheres to the surface of the implant upon deployment of the implant.

15523. The method of item 15349, wherein the device has a coating, and wherein the coating is stable at room temperature for a period of 1 year.

15524. The method of item 15349, wherein the device has a coating, and wherein the anti-scarring agent is present in the coating in an amount ranging between about 0.0001% to about 1% by weight.

15525. The method of item 15349, wherein the device has a coating, and wherein the anti-scarring agent is present in the coating in an amount ranging between about 1% to about 10% by weight.

15526. The method of item 15349, wherein the device has a coating, and wherein the anti-scarring agent is present in the coating in an amount ranging between about 10% to about 25% by weight.

15527. The method of item 15349, wherein the device has a coating, and wherein the anti-scarring agent is present in the coating in an amount ranging between about 25% to about 70% by weight.

15528. The method of item 15349, wherein the device has a coating, and wherein the coating further comprises a polymer.

15529. The method of item 15349, wherein the device has a first coating having a first composition and a second coating having a second composition.

15530. The method of item 15349, wherein the device has a first coating having a first composition and a second coating having a second composition, wherein the first composition and the second composition are different.

15531. The method of item 15349, wherein the composition comprises a polymer.

15532. The method of item 15349, wherein the composition comprises a polymeric carrier.

15533. The method of item 15349, wherein the composition comprises a polymeric carrier, and wherein the polymeric carrier comprises a copolymer.

15534. The method of item 15349, wherein the composition comprises a polymeric carrier, and wherein the polymeric carrier comprises a block copolymer.

15535. The method of item 15349, wherein the composition comprises a polymeric carrier, and wherein the polymeric carrier comprises a random copolymer.

15536. The method of item 15349, wherein the composition comprises a polymeric carrier, and wherein the polymeric carrier comprises a biodegradable polymer.

15537. The method of item 15349, wherein the composition comprises a polymeric carrier, and wherein the polymeric carrier comprises a non-biodegradable polymer.

15538. The method of item 15349, wherein the composition comprises a polymeric carrier, and wherein the polymeric carrier comprises a hydrophilic polymer.

15539. The method of item 15349, wherein the composition comprises a polymeric carrier, and wherein the polymeric carrier comprises a hydrophobic polymer.

15540. The method of item 15349, wherein the composition comprises a polymeric carrier, and wherein the polymeric carrier comprises a polymer having hydrophilic domains.

15541. The method of item 15349, wherein the composition comprises a polymeric carrier, and wherein the polymeric carrier comprises a polymer having hydrophobic domains.

15542. The method of item 15349, wherein the composition comprises a polymeric carrier, and wherein the polymeric carrier comprises a non-conductive polymer.

15543. The method of item 15349, wherein the composition comprises a polymeric carrier, and wherein the polymeric carrier comprises an elastomer.

15544. The method of item 15349, wherein the composition comprises a polymeric carrier, and wherein the polymeric carrier comprises a hydrogel.

15545. The method of item 15349, wherein the composition comprises a polymeric carrier, and wherein the polymeric carrier comprises a silicone polymer.

15546. The method of item 15349, wherein the composition comprises a polymeric carrier, and wherein the polymeric carrier comprises a hydrocarbon polymer.

15547. The method of item 15349, wherein the composition comprises a polymeric carrier, and wherein the polymeric carrier comprises a styrene-derived polymer.

15548. The method of item 15349, wherein the composition comprises a polymeric carrier, and wherein the polymeric carrier comprises a butadiene polymer.

15549. The method of item 15349, wherein the composition comprises a polymeric carrier, and wherein the polymeric carrier comprises a macromer.

15550. The method of item 15349, wherein the composition comprises a polymeric carrier, and wherein the polymeric carrier comprises a poly(ethylene glycol)polymer.

15551. The method of item 15349 wherein the composition comprises a polymeric carrier, and wherein the polymeric carrier comprises an amorphous polymer.

15552. The method of item 15349, wherein the device comprises a lubricious coating.

15553. The method of item 15349 wherein the anti-scarring agent is located within pores or holes of the device.

15554. The method of item 15349 wherein the anti-scarring agent is located within a channel, lumen, or divet of the device.

15555. The method of item 15349, wherein the device comprises a second pharmaceutically active agent.

15556. The method of item 15349 wherein the device comprises an anti-inflammatory agent.

15557. The method of item 15349 wherein the device comprises an agent that inhibits infection.

15558. The method of item 15349 wherein the device comprises an agent that inhibits infection, and wherein the agent is an anthracycline.

15559. The method of item 15349 wherein the device comprises an agent that inhibits infection, and wherein the agent is doxorubicin.

15560. The method of item 15349 wherein the device comprises an agent that inhibits infection, and wherein the agent is mitoxantrone.

15561. The method of item 15349 wherein the device comprises an agent that inhibits infection, and wherein the agent is a fluoropyrimidine.

15562. The method of item 15349 wherein the device comprises an agent that inhibits infection, and wherein the agent is 5-fluorouracil (5-FU).

15563. The method of item 15349 wherein the device comprises an agent that inhibits infection, and wherein the agent is a folic acid antagonist.

15564. The method of item 15349 wherein the device comprises an agent that inhibits infection, and wherein the agent is methotrexate.

15565. The method of item 15349 wherein the device comprises an agent that inhibits infection, and wherein the agent is a podophylotoxin.

15566. The method of item 15349 wherein the device comprises an agent that inhibits infection, and wherein the agent is etoposide.

15567. The method of item 15349 wherein the device comprises an agent that inhibits infection, and wherein the agent is a camptothecin.

15568. The method of item 15349 wherein the device comprises an agent that inhibits infection, and wherein the agent is a hydroxyurea.

15569. The method of item 15349 wherein the device comprises an agent that inhibits infection, and wherein the agent is a platinum complex.

15570. The method of item 15349 wherein the device comprises an agent that inhibits infection, and wherein the agent is cisplatin.

15571. The method of item 15349, further comprising an anti-thrombotic agent.

15572. The method of item 15349 wherein the device comprises a visualization agent.

15573. The method of item 15349 wherein the device comprises a visualization agent, wherein the visualization agent is a radiopaque material, and wherein the radiopaque material comprises a metal, a halogenated compound, or a barium containing compound.

15574. The method of item 15349 wherein the device comprises a visualization agent, wherein the visualization agent is a radiopaque material, and wherein the radiopaque material comprises barium, tantalum, or technetium.

15575. The method of item 15349 wherein the device comprises a visualization agent, and wherein the visualization agent is a MRI responsive material.

15576. The method of item 15349 wherein the device comprises a visualization agent, and wherein the visualization agent comprises a gadolinium chelate.

15577. The method of item 15349 wherein the device comprises a visualization agent, and wherein the visualization agent comprises iron, magnesium, manganese, copper, or chromium.

15578. The method of item 15349 wherein the device comprises a visualization agent, and wherein the visualization agent comprises an iron oxide compound.

15579. The method of item 15349 wherein the device comprises a visualization agent, and wherein the visualization agent comprises a dye, pigment, or colorant.

15580. The method of item 15349 wherein the device comprises an echogenic material.

15581. The method of item 15349 wherein the device comprises an echogenic material, and wherein the echogenic material is in the form of a coating.

15582. The method of item 15349 wherein the device is sterile.

15583. The method of item 15349 wherein the anti-scarring agent is released into tissue in the vicinity of the device after deployment of the device.

15584. The method of item 15349 wherein the anti-scarring agent is released into tissue in the vicinity of the device after deployment of the device, and wherein the tissue is connective tissue.

15585. The method of item 15349 wherein the anti-scarring agent is released into tissue in the vicinity of the device after deployment of the device, and wherein the tissue is muscle tissue.

15586. The method of item 15349 wherein the anti-scarring agent is released into tissue in the vicinity of the device after deployment of the device, and wherein the tissue is nerve tissue.

15587. The method of item 15349 wherein the anti-scarring agent is released into tissue in the vicinity of the device after deployment of the device, and wherein the tissue is epithelium tissue.

15588. The method of item 15349 wherein the anti-scarring agent is released in effective concentrations from the device over a period ranging from the time of deployment of the device to about 1 year.

15589. The method of item 15349 wherein the anti-scarring agent is released in effective concentrations from the device over a period ranging from about 1 month to 6 months.

15590. The method of item 15349 wherein the anti-scarring agent is released in effective concentrations from the device over a period ranging from about 1-90 days.

15591. The method of item 15349 wherein the anti-scarring agent is released in effective concentrations from the device at a constant rate.

15592. The method of item 15349 wherein the anti-scarring agent is released in effective concentrations from the device at an increasing rate.

15593. The method of item 15349 wherein the anti-scarring agent is released in effective concentrations from the device at a decreasing rate.

15594. The method of item 15349 wherein the anti-scarring agent is released in effective concentrations from the composition comprising the anti-scarring agent by diffusion over a period ranging from the time of deployment of the device to about 90 days.

15595. The method of item 15349 wherein the anti-scarring agent is released in effective concentrations from the composition comprising the anti-scarring agent by erosion of the composition over a period ranging from the time of deployment of the device to about 90 days.

15596. The method of item 15349 wherein the device comprises about 0.01 μg to about 10 μg of the anti-scarring agent.

15597. The method of item 15349 wherein the device comprises about 10 μg to about 10 mg of the anti-scarring agent.

15598. The method of item 15349 wherein the device comprises about 10 mg to about 250 mg of the anti-scarring agent.

15599. The method of item 15349 wherein the device comprises about 250 mg to about 1000 mg of the anti-scarring agent.

15600. The method of item 15349 wherein the device comprises about 1000 mg to about 2500 mg of the anti-scarring agent.

15601. The method of item 15349 wherein a surface of the device comprises less than 0.01 μg of the anti-scarring agent per mm² of device surface to which the anti-scarring agent is applied.

15602. The method of item 15349 wherein a surface of the device comprises about 0.01 μg to about 1 μg of the anti-scarring agent per mm² of device surface to which the anti-scarring agent is applied.

15603. The method of item 15349 wherein a surface of the device comprises about 1 μg to about 10 μg of the anti-scarring agent per mm² of device surface to which the anti-scarring agent is applied.

15604. The method of item 15349 wherein a surface of the device comprises about 10 μg to about 250 μg of the anti-scarring agent per mm² of device surface to which the anti-scarring agent is applied.

15605. The method of item 15349 wherein a surface of the device comprises about 250 μg to about 1000 μg of the anti-scarring agent of anti-scarring agent per mm² of device surface to which the anti-scarring agent is applied.

15606. The method of item 15349 wherein a surface of the device comprises about 1000 μg to about 2500 μg of the anti-scarring agent per mm² of device surface to which the anti-scarring agent is applied.

15607. The method of item 15349 wherein the combining is performed by direct affixing the agent or the composition to the implant.

15608. The method of item 15349 wherein the combining is performed by spraying the agent or the component onto the implant.

15609. The method of item 15349 wherein the combining is performed by electrospraying the agent or the composition onto the implant.

15610. The method of item 15349 wherein the combining is performed by dipping the implant into a solution comprising the agent or the composition.

15611. The method of item 15349 wherein the combining is performed by covalently attaching the agent or the composition to the implant.

15612. The method of item 15349 wherein the combining is performed by non-covalently attaching the agent or the composition to the implant.

15613. The method of item 15349 wherein the combining is performed by coating the implant with a substance that contains the agent or the composition.

15614. The method of item 15349 wherein the combining is performed by coating the implant with a substance that absorbs the agent.

15615. The method of item 15349 wherein the combining is performed by interweaving a thread composed of, or coated with, the agent or the composition.

15616. The method of item 15349 wherein the combining is performed by covering all the implant with a sleeve that contains the agent or the composition.

15617. The method of item 15349 wherein the combining is performed by covering a portion of the implant with a sleeve that contains the agent or the composition.

15618. The method of item 15349 wherein the combining is performed by covering all the implant with a cover that contains the agent or the composition.

15619. The method of item 15349 wherein the combining is performed by covering a portion of the implant with a cover that contains the agent or the composition.

15620. The method of item 15349 wherein the combining is performed by covering all the implant with an electrospun fabric that contains the agent or the composition.

15621. The method of item 15349 wherein the combining is performed by covering a portion of the implant with an electrospun fabric that contains the agent or the composition.

15622. The method of item 15349 wherein the combining is performed by covering all the implant with a mesh that contains the agent or the composition.

15623. The method of item 15349 wherein the combining is performed by covering a portion of the implant with a mesh that contains the agent or the composition.

15624. The method of item 15349 wherein the combining is performed by constructing all the implant with the agent or the composition.

15625. The method of item 15349 wherein the combining is performed by constructing a portion of the implant with the agent or the composition.

15626. The method of item 15349 wherein the combining is performed by impregnating the implant with the agent or the composition.

15627. The method of item 15349 wherein the combining is performed by constructing all of the implant from a degradable polymer that releases the agent.

15628. The method of item 15349 wherein the combining is performed by constructing a portion of the implant from a degradable polymer that releases the agent.

15629. The method of item 15349 wherein the combining is performed by dipping the implant into a solution that comprise the agent and an inert solvent for the implant.

15630. The method of item 15349 wherein the combining is performed by dipping the implant into a solution that comprises the agent and a solvent that will swill the implant.

15631. The method of item 15349 wherein the combining is performed by dipping the implant into a solution that comprises the agent and a solvent that will dissolve the implant.

15632. The method of item 15349 wherein the combining is performed by dipping the implant into a solution that comprises the agent, a polymer and an inert solvent for the implant.

15633. The method of item 15349 wherein the combining is performed by dipping the implant into a solution that comprises the agent, a polymer and a solvent that will swill the implant.

15634. The method of item 15349 wherein the combining is performed by dipping the implant into a solution that comprises the agent, a polymer and a solvent that will dissolve the implant.

15635. The method of item 15349 wherein the combining is performed by spraying the implant into a solution that comprises the agent and an inert solvent for the implant.

15636. The method of item 15349 wherein the combining is performed by spraying the implant into a solution that comprises the agent and a solvent that will swill the implant.

15637. The method of item 15349 wherein the combining is performed by spraying the implant into a solution that comprises the agent and a solvent that will dissolve the implant.

15638. The method of item 15349 wherein the combining is performed by spraying the implant into a solution that comprises the agent, a polymer and an inert solvent for the implant.

15639. The method of item 15349 wherein the combining is performed by spraying the implant into a solution that comprises the agent, a polymer and a solvent that will swill the implant.

15640. The method of item 15349 wherein the combining is performed by spraying the implant into a solution that comprises the agent, a polymer and a solvent that will dissolve the implant.

15641. The method of item 15349 wherein the implant is a spinal disc.

15642. The method of item 15349 wherein the implant is a vertebral disc prosthesis.

15643. The method of item 15349 wherein the implant is an intervertebral disc.

15644. The method of item 15349 wherein the implant is a partial spinal prosthesis.

15645. The method of item 15349 wherein the implant is a spinal nucleus implant.

15646. The method of item 15349 wherein the implant is an intervertebral disc spacer.

15647. The method of item 15349 wherein the implant is a fusion cage.

15648. The method of item 15349 wherein the implant is a fusion basket.

15649. The method of item 15349 wherein the implant is a fusion chamber.

15650. The method of item 15349 wherein the implant is a spinal anchoring device.

15651. The method of item 15349 wherein the implant is a bone fixation device.

15652. The method of item 15349 wherein the implant is an anchoring bone plate for the spine.

15653. The method of item 15349 wherein the implant is an anchoring screw for the spine.

15654. The method of item 15349 wherein the implant is an implantable rod for the spine.

15655. The method of item 15349 wherein the implant is an implantable dowel for the spine.

15656. The method of item 15349 wherein the implant is an implantable hook for the spine.

15657. The method of item 15349 wherein the implant is a wire for spinal binding.

15658. The method of item 15349 wherein the implant is a wedge for spinal support.

15659. A method of making a medical device comprising: combining a pressure monitoring implant and an anti-scarring agent or a composition comprising an anti-scarring agent, wherein the agent inhibits scarring between the device and a host into which the device is implanted.

15660. The method of item 15659 wherein the agent inhibits cell regeneration.

15661. The method of item 15659 wherein the agent inhibits angiogenesis.

15662. The method of item 15659 wherein the agent inhibits fibroblast migration.

15663. The method of item 15659 wherein the agent inhibits fibroblast proliferation.

15664. The method of item 15659 wherein the agent inhibits deposition of extracellular matrix.

15665. The method of item 15659 wherein the agent inhibits tissue remodeling.

15666. The method of item 15659 wherein the agent is an angiogenesis inhibitor.

15667. The method of item 15659 wherein the agent is a 5-lipoxygenase inhibitor or antagonist.

15668. The method of item 15659 wherein the agent is a chemokine receptor antagonist.

15669. The method of item 15659 wherein the agent is a cell cycle inhibitor.

15670. The method of item 15659 wherein the agent is a taxane.

15671. The method of item 15659 wherein the agent is an anti-microtubule agent.

15672. The method of item 15659 wherein the agent is paclitaxel.

15673. The method of item 15659 wherein the agent is not paclitaxel.

15674. The method of item 15659 wherein the agent is an analogue or derivative of paclitaxel.

15675. The method of item 15659 wherein the agent is a vinca alkaloid.

15676. The method of item 15659 wherein the agent is camptothecin or an analogue or derivative thereof.

15677. The method of item 15659 wherein the agent is a podophyllotoxin.

15678. The method of item 15659 wherein the agent is a podophyllotoxin, wherein the podophyllotoxin is etoposide or an analogue or derivative thereof.

15679. The method of item 15659 wherein the agent is an anthracycline.

15680. The method of item 15659 wherein the agent is an anthracycline, wherein the anthracycline is doxorubicin or an analogue or derivative thereof.

15681. The method of item 15659 wherein the agent is an anthracycline, wherein the anthracycline is mitoxantrone or an analogue or derivative thereof.

15682. The method of item 15659 wherein the agent is a platinum compound.

15683. The method of item 15659 wherein the agent is a nitrosourea.

15684. The method of item 15659 wherein the agent is a nitroimidazole.

15685. The method of item 15659 wherein the agent is a folic acid antagonist.

15686. The method of item 15659 wherein the agent is a cytidine analogue.

15687. The method of item 15659 wherein the agent is a pyrimidine analogue.

15688. The method of item 15659 wherein the agent is a fluoropyrimidine analogue.

15689. The method of item 15659 wherein the agent is a purine analogue.

15690. The method of item 15659 wherein the agent is a nitrogen mustard or an analogue or derivative thereof.

15691. The method of item 15659 wherein the agent is a hydroxyurea.

15692. The method of item 15659 wherein the agent is a mytomicin or an analogue or derivative thereof.

15693. The method of item 15659 wherein the agent is an alkyl sulfonate.

15694. The method of item 15659 wherein the agent is a benzamide or an analogue or derivative thereof.

15695. The method of item 15659 wherein the agent is a nicotinamide or an analogue or derivative thereof.

15696. The method of item 15659 wherein the agent is a halogenated sugar or an analogue or derivative thereof.

15697. The method of item 15659 wherein the agent is a DNA alkylating agent.

15698. The method of item 15659 wherein the agent is an anti-microtubule agent.

15699. The method of item 15659 wherein the agent is a topoisomerase inhibitor.

15700. The method of item 15659 wherein the agent is a DNA cleaving agent.

15701. The method of item 15659 wherein the agent is an antimetabolite.

15702. The method of item 15659 wherein the agent inhibits adenosine deaminase.

15703. The method of item 15659 wherein the agent inhibits purine ring synthesis.

15704. The method of item 15659 wherein the agent is a nucleotide interconversion inhibitor.

15705. The method of item 15659 wherein the agent inhibits dihydrofolate reduction.

15706. The method of item 15659 wherein the agent blocks thymidine monophosphate.

15707. The method of item 15659 wherein the agent causes DNA damage.

15708. The method of item 15659 wherein the agent is a DNA intercalation agent.

15709. The method of item 15659 wherein the agent is a RNA synthesis inhibitor.

15710. The method of item 15659 wherein the agent is a pyrimidine synthesis inhibitor.

15711. The method of item 15659 wherein the agent inhibits ribonucleotide synthesis or function.

15712. The method of item 15659 wherein the agent inhibits thymidine monophosphate synthesis or function.

15713. The method of item 15659 wherein the agent inhibits DNA synthesis.

15714. The method of item 15659 wherein the agent causes DNA adduct formation.

15715. The method of item 15659 wherein the agent inhibits protein synthesis.

15716. The method of item 15659 wherein the agent inhibits microtubule function.

15717. The method of item 15659 wherein the agent is a cyclin dependent protein kinase inhibitor.

15718. The method of item 15659 wherein the agent is an epidermal growth factor kinase inhibitor.

15719. The method of item 15659 wherein the agent is an elastase inhibitor.

15720. The method of item 15659 wherein the agent is a factor Xa inhibitor.

15721. The method of item 15659 wherein the agent is a farnesyltransferase inhibitor.

15722. The method of item 15659 wherein the agent is a fibrinogen antagonist.

15723. The method of item 15659 wherein the agent is a guanylate cyclase stimulant.

15724. The method of item 15659 wherein the agent is a heat shock protein 90 antagonist.

15725. The method of item 15659 wherein the agent is a heat shock protein 90 antagonist, wherein the heat shock protein 90 antagonist is geldanamycin or an analogue or derivative thereof.

15726. The method of item 15659 wherein the agent is a guanylate cyclase stimulant.

15727. The method of item 15659 wherein the agent is a HMGCoA reductase inhibitor.

15728. The method of item 15659 wherein the agent is a HMGCoA reductase inhibitor, wherein the HMGCoA reductase inhibitor is simvastatin or an analogue or derivative thereof.

15729. The method of item 15659 wherein the agent is a hydroorotate dehydrogenase inhibitor.

15730. The method of item 15659 wherein the agent is an IKK2 inhibitor.

15731. The method of item 15659 wherein the agent is an IL-1 antagonist.

15732. The method of item 15659 wherein the agent is an ICE antagonist.

15733. The method of item 15659 wherein the agent is an IRAK antagonist.

15734. The method of item 15659 wherein the agent is an IL-4 agonist.

15735. The method of item 15659 wherein the agent is an immunomodulatory agent.

15736. The method of item 15659 wherein the agent is sirolimus or an analogue or derivative thereof.

15737. The method of item 15659 wherein the agent is not sirolimus.

15738. The method of item 15659 wherein the agent is everolimus or an analogue or derivative thereof.

15739. The method of item 15659 wherein the agent is tacrolimus or an analogue or derivative thereof.

15740. The method of item 15659 wherein the agent is not tacrolimus.

15741. The method of item 15659 wherein the agent is biolmus or an analogue or derivative thereof.

15742. The method of item 15659 wherein the agent is tresperimus or an analogue or derivative thereof.

15743. The method of item 15659 wherein the agent is auranofin or an analogue or derivative thereof.

15744. The method of item 15659 wherein the agent is 27-0-demethylrapamycin or an analogue or derivative thereof.

15745. The method of item 15659 wherein the agent is gusperimus or an analogue or derivative thereof.

15746. The method of item 15659 wherein the agent is pimecrolimus or an analogue or derivative thereof.

15747. The method of item 15659 wherein the agent is ABT-578 or an analogue or derivative thereof.

15748. The method of item 15659 wherein the agent is an inosine monophosphate dehydrogenase (IMPDH) inhibitor.

15749. The method of item 15659 wherein the agent is an IMPDH inhibitor, wherein the IMPDH inhibitor is mycophenolic acid or an analogue or derivative thereof.

15750. The method of item 15659 wherein the agent is an IMPDH inhibitor, wherein the IMPDH inhibitor is 1-alpha-25 dihydroxy vitamin D₃ or an analogue or derivative thereof.

15751. The method of item 15659 wherein the agent is a leukotriene inhibitor.

15752. The method of item 15659 wherein the agent is a MCP-1 antagonist.

15753. The method of item 15659 wherein the agent is a MMP inhibitor.

15754. The method of item 15659 wherein the agent is an NF kappa B inhibitor.

15755. The method of item 15659 wherein the agent is an NF kappa B inhibitor, wherein the NF kappa B inhibitor is Bay 11-7082.

15756. The method of item 15659 wherein the agent is an NO agonist.

15757. The method of item 15659 wherein the agent is a p38 MAP kinase inhibitor.

15758. The method of item 15659 wherein the agent is a p38 MAP kinase inhibitor, wherein the p38 MAP kinase inhibitor is SB 202190.

15759. The method of item 15659 wherein the agent is a phosphodiesterase inhibitor.

15760. The method of item 15659 wherein the agent is a TGF beta inhibitor.

15761. The method of item 15659 wherein the agent is a thromboxane A2 antagonist.

15762. The method of item 15659 wherein the agent is a TNFa antagonist.

15763. The method of item 15659 wherein the agent is a TACE inhibitor.

15764. The method of item 15659 wherein the agent is a tyrosine kinase inhibitor.

15765. The method of item 15659 wherein the agent is a vitronectin inhibitor.

15766. The method of item 15659 wherein the agent is a fibroblast growth factor inhibitor.

15767. The method of item 15659 wherein the agent is a protein kinase inhibitor.

15768. The method of item 15659 wherein the agent is a PDGF receptor kinase inhibitor.

15769. The method of item 15659 wherein the agent is an endothelial growth factor receptor kinase inhibitor.

15770. The method of item 15659 wherein the agent is a retinoic acid receptor antagonist.

15771. The method of item 15659 wherein the agent is a platelet derived growth factor receptor kinase inhibitor.

15772. The method of item 15659 wherein the agent is a fibronogin antagonist.

15773. The method of item 15659 wherein the agent is an antimycotic agent.

15774. The method of item 15659 wherein the agent is an antimycotic agent, wherein the antimycotic agent is sulconizole.

15775. The method of item 15659 wherein the agent is a bisphosphonate.

15776. The method of item 15659 wherein the agent is a phospholipase A1 inhibitor.

15777. The method of item 15659 wherein the agent is a histamine H1/H2/H3 receptor antagonist.

15778. The method of item 15659 wherein the agent is a macrolide antibiotic.

15779. The method of item 15659 wherein the agent is a GPIIb/IIIa receptor antagonist.

15780. The method of item 15659 wherein the agent is an endothelin receptor antagonist.

15781. The method of item 15659 wherein the agent is a peroxisome proliferator-activated receptor agonist.

15782. The method of item 15659 wherein the agent is an estrogen receptor agent.

15783. The method of item 15659 wherein the agent is a somastostatin analogue.

15784. The method of item 15659 wherein the agent is a neurokinin 1 antagonist.

15785. The method of item 15659 wherein the agent is a neurokinin 3 antagonist.

15786. The method of item 15659 wherein the agent is a VLA-4 antagonist.

15787. The method of item 15659 wherein the agent is an osteoclast inhibitor.

15788. The method of item 15659 wherein the agent is a DNA topoisomerase ATP hydrolyzing inhibitor.

15789. The method of item 15659 wherein the agent is an angiotensin I converting enzyme inhibitor.

15790. The method of item 15659 wherein the agent is an angiotensin II antagonist.

15791. The method of item 15659 wherein the agent is an enkephalinase inhibitor.

15792. The method of item 15659 wherein the agent is a peroxisome proliferator-activated receptor gamma agonist insulin sensitizer.

15793. The method of item 15659 wherein the agent is a protein kinase C inhibitor.

15794. The method of item 15659 wherein the agent is a ROCK (rho-associated kinase) inhibitor.

15795. The method of item 15659 wherein the agent is a CXCR3 inhibitor.

15796. The method of item 15659 wherein the agent is an Itk inhibitor.

15797. The method of item 15659 wherein the agent is a cytosolic phospholipase A₂-alpha inhibitor.

15798. The method of item 15659 wherein the agent is a PPAR agonist.

15799. The method of item 15659 wherein the agent is an immunosuppressant.

15800. The method of item 15659 wherein the agent is an Erb inhibitor.

15801. The method of item 15659 wherein the agent is an apoptosis agonist.

15802. The method of item 15659 wherein the agent is a lipocortin agonist.

15803. The method of item 15659 wherein the agent is a VCAM-1 antagonist.

15804. The method of item 15659 wherein the agent is a collagen antagonist.

15805. The method of item 15659 wherein the agent is an alpha 2 integrin antagonist.

15806. The method of item 15659 wherein the agent is a TNF alpha inhibitor.

15807. The method of item 15659 wherein the agent is a nitric oxide inhibitor.

15808. The method of item 15659 wherein the agent is a cathepsin inhibitor.

15809. The method of item 15659 wherein the agent is not an anti-inflammatory agent.

15810. The method of item 15659 wherein the agent is not a steroid.

15811. The method of item 15659 wherein the agent is not a glucocorticosteroid.

15812. The method of item 15659 wherein the agent is not dexamethasone.

15813. The method of item 15659 wherein the agent is not an anti-infective agent.

15814. The method of item 15659 wherein the agent is not an antibiotic.

15815. The method of item 15659 wherein the agent is not an anti-fungal agent.

15816. The method of item 15659, wherein the composition comprises a polymer.

15817. The method of item 15659, wherein the composition comprises a polymeric carrier.

15818. The method of item 15659 wherein the anti-scarring agent inhibits adhesion between the device and a host into which the device is implanted.

15819. The method of item 15659 wherein the device delivers the anti-scarring agent locally to tissue proximate to the device.

15820. The method of item 15659 wherein the device has a coating that comprises the anti-scarring agent.

15821. The method of item 15659, wherein the device has a coating that comprises the agent and is disposed on a surface of the implant.

15822. The method of item 15659, wherein the device has a coating that comprises the agent and directly contacts the implant.

15823. The method of item 15659, wherein the device has a coating that comprises the agent and indirectly contacts the implant.

15824. The method of item 15659, wherein the device has a coating that comprises the agent and partially covers the implant.

15825. The method of item 15659, wherein the device has a coating that comprises the agent and completely covers the implant.

15826. The method of item 15659, wherein the device has a uniform coating.

15827. The method of item 15659, wherein the device has a non-uniform coating.

15828. The method of item 15659, wherein the device has a discontinuous coating.

15829. The method of item 15659, wherein the device has a patterned coating.

15830. The method of item 15659, wherein the device has a coating with a thickness of 100 μm or less.

15831. The method of item 15659, wherein the device has a coating with a thickness of 10 μm or less.

15832. The method of item 15659, wherein the device has a coating, and the coating adheres to the surface of the implant upon deployment of the implant.

15833. The method of item 15659, wherein the device has a coating, and wherein the coating is stable at room temperature for a period of 1 year.

15834. The method of item 15659, wherein the device has a coating, and wherein the anti-scarring agent is present in the coating in an amount ranging between about 0.0001% to about 1% by weight.

15835. The method of item 15659, wherein the device has a coating, and wherein the anti-scarring agent is present in the coating in an amount ranging between about 1% to about 10% by weight.

15836. The method of item 15659, wherein the device has a coating, and wherein the anti-scarring agent is present in the coating in an amount ranging between about 10% to about 25% by weight.

15837. The method of item 15659, wherein the device has a coating, and wherein the anti-scarring agent is present in the coating in an amount ranging between about 25% to about 70% by weight.

15838. The method of item 15659, wherein the device has a coating, and wherein the coating further comprises a polymer.

15839. The method of item 15659, wherein the device has a first coating having a first composition and a second coating having a second composition.

15840. The method of item 15659, wherein the device has a first coating having a first composition and a second coating having a second composition, wherein the first composition and the second composition are different.

15841. The method of item 15659, wherein the composition comprises a polymer.

15842. The method of item 15659, wherein the composition comprises a polymeric carrier.

15843. The method of item 15659, wherein the composition comprises a polymeric carrier, and wherein the polymeric carrier comprises a copolymer.

15844. The method of item 15659, wherein the composition comprises a polymeric carrier, and wherein the polymeric carrier comprises a block copolymer.

15845. The method of item 15659, wherein the composition comprises a polymeric carrier, and wherein the polymeric carrier comprises a random copolymer.

15846. The method of item 15659, wherein the composition comprises a polymeric carrier, and wherein the polymeric carrier comprises a biodegradable polymer.

15847. The method of item 15659, wherein the composition comprises a polymeric carrier, and wherein the polymeric carrier comprises a non-biodegradable polymer.

15848. The method of item 15659, wherein the composition comprises a polymeric carrier, and wherein the polymeric carrier comprises a hydrophilic polymer.

15849. The method of item 15659, wherein the composition comprises a polymeric carrier, and wherein the polymeric carrier comprises a hydrophobic polymer.

15850. The method of item 15659, wherein the composition comprises a polymeric carrier, and wherein the polymeric carrier comprises a polymer having hydrophilic domains.

15851. The method of item 15659, wherein the composition comprises a polymeric carrier, and wherein the polymeric carrier comprises a polymer having hydrophobic domains.

15852. The method of item 15659, wherein the composition comprises a polymeric carrier, and wherein the polymeric carrier comprises a non-conductive polymer.

15853. The method of item 15659, wherein the composition comprises a polymeric carrier, and wherein the polymeric carrier comprises an elastomer.

15854. The method of item 15659, wherein the composition comprises a polymeric carrier, and wherein the polymeric carrier comprises a hydrogel.

15855. The method of item 15659, wherein the composition comprises a polymeric carrier, and wherein the polymeric carrier comprises a silicone polymer.

15856. The method of item 15659, wherein the composition comprises a polymeric carrier, and wherein the polymeric carrier comprises a hydrocarbon polymer.

15857. The method of item 15659, wherein the composition comprises a polymeric carrier, and wherein the polymeric carrier comprises a styrene-derived polymer.

15858. The method of item 15659, wherein the composition comprises a polymeric carrier, and wherein the polymeric carrier comprises a butadiene polymer.

15859. The method of item 15659, wherein the composition comprises a polymeric carrier, and wherein the polymeric carrier comprises a macromer.

15860. The method of item 15659, wherein the composition comprises a polymeric carrier, and wherein the polymeric carrier comprises a poly(ethylene glycol)polymer.

15861. The method of item 15659 wherein the composition comprises a polymeric carrier, and wherein the polymeric carrier comprises an amorphous polymer.

15862. The method of item 15659, wherein the device comprises a lubricious coating.

15863. The method of item 15659 wherein the anti-scarring agent is located within pores or holes of the device.

15864. The method of item 15659 wherein the anti-scarring agent is located within a channel, lumen, or divet of the device.

15865. The method of item 15659, wherein the device comprises a second pharmaceutically active agent.

15866. The method of item 15659 wherein the device comprises an anti-inflammatory agent.

15867. The method of item 15659 wherein the device comprises an agent that inhibits infection.

15868. The method of item 15659 wherein the device comprises an agent that inhibits infection, and wherein the agent is an anthracycline.

15869. The method of item 15659 wherein the device comprises an agent that inhibits infection, and wherein the agent is doxorubicin.

15870. The method of item 15659 wherein the device comprises an agent that inhibits infection, and wherein the agent is mitoxantrone.

15871. The method of item 15659 wherein the device comprises an agent that inhibits infection, and wherein the agent is a fluoropyrimidine.

15872. The method of item 15659 wherein the device comprises an agent that inhibits infection, and wherein the agent is 5-fluorouracil (5-FU).

15873. The method of item 15659 wherein the device comprises an agent that inhibits infection, and wherein the agent is a folic acid antagonist.

15874. The method of item 15659 wherein the device comprises an agent that inhibits infection, and wherein the agent is methotrexate.

15875. The method of item 15659 wherein the device comprises an agent that inhibits infection, and wherein the agent is a podophylotoxin.

15876. The method of item 15659 wherein the device comprises an agent that inhibits infection, and wherein the agent is etoposide.

15877. The method of item 15659 wherein the device comprises an agent that inhibits infection, and wherein the agent is a camptothecin.

15878. The method of item 15659 wherein the device comprises an agent that inhibits infection, and wherein the agent is a hydroxyurea.

15879. The method of item 15659 wherein the device comprises an agent that inhibits infection, and wherein the agent is a platinum complex.

15880. The method of item 15659 wherein the device comprises an agent that inhibits infection, and wherein the agent is cisplatin.

15881. The method of item 15659, further comprising an anti-thrombotic agent.

15882. The method of item 15659 wherein the device comprises a visualization agent.

15883. The method of item 15659 wherein the device comprises a visualization agent, wherein the visualization agent is a radiopaque material, and wherein the radiopaque material comprises a metal, a halogenated compound, or a barium containing compound.

15884. The method of item 15659 wherein the device comprises a visualization agent, wherein the visualization agent is a radiopaque material, and wherein the radiopaque material comprises barium, tantalum, or technetium.

15885. The method of item 15659 wherein the device comprises a visualization agent, and wherein the visualization agent is a MRI responsive material.

15886. The method of item 15659 wherein the device comprises a visualization agent, and wherein the visualization agent comprises a gadolinium chelate.

15887. The method of item 15659 wherein the device comprises a visualization agent, and wherein the visualization agent comprises iron, magnesium, manganese, copper, or chromium.

15888. The method of item 15659 wherein the device comprises a visualization agent, and wherein the visualization agent comprises an iron oxide compound.

15889. The method of item 15659 wherein the device comprises a visualization agent, and wherein the visualization agent comprises a dye, pigment, or colorant.

15890. The method of item 15659 wherein the device comprises an echogenic material.

15891. The method of item 15659 wherein the device comprises an echogenic material, and wherein the echogenic material is in the form of a coating.

15892. The method of item 15659 wherein the device is sterile.

15893. The method of item 15659 wherein the anti-scarring agent is released into tissue in the vicinity of the device after deployment of the device.

15894. The method of item 15659 wherein the anti-scarring agent is released into tissue in the vicinity of the device after deployment of the device, and wherein the tissue is connective tissue.

15895. The method of item 15659 wherein the anti-scarring agent is released into tissue in the vicinity of the device after deployment of the device, and wherein the tissue is muscle tissue.

15896. The method of item 15659 wherein the anti-scarring agent is released into tissue in the vicinity of the device after deployment of the device, and wherein the tissue is nerve tissue.

15897. The method of item 15659 wherein the anti-scarring agent is released into tissue in the vicinity of the device after deployment of the device, and wherein the tissue is epithelium tissue.

15898. The method of item 15659 wherein the anti-scarring agent is released in effective concentrations from the device over a period ranging from the time of deployment of the device to about 1 year.

15899. The method of item 15659 wherein the anti-scarring agent is released in effective concentrations from the device over a period ranging from about 1 month to 6 months.

15900. The method of item 15659 wherein the anti-scarring agent is released in effective concentrations from the device over a period ranging from about 1-90 days.

15901. The method of item 15659 wherein the anti-scarring agent is released in effective concentrations from the device at a constant rate.

15902. The method of item 15659 wherein the anti-scarring agent is released in effective concentrations from the device at an increasing rate.

15903. The method of item 15659 wherein the anti-scarring agent is released in effective concentrations from the device at a decreasing rate.

15904. The method of item 15659 wherein the anti-scarring agent is released in effective concentrations from the composition comprising the anti-scarring agent by diffusion over a period ranging from the time of deployment of the device to about 90 days.

15905. The method of item 15659 wherein the anti-scarring agent is released in effective concentrations from the composition comprising the anti-scarring agent by erosion of the composition over a period ranging from the time of deployment of the device to about 90 days.

15906. The method of item 15659 wherein the device comprises about 0.01 μg to about 10 μg of the anti-scarring agent.

15907. The method of item 15659 wherein the device comprises about 10 μg to about 10 mg of the anti-scarring agent.

15908. The method of item 15659 wherein the device comprises about 10 mg to about 250 mg of the anti-scarring agent.

15909. The method of item 15659 wherein the device comprises about 250 mg to about 1000 mg of the anti-scarring agent.

15910. The method of item 15659 wherein the device comprises about 1000 mg to about 2500 mg of the anti-scarring agent.

15911. The method of item 15659 wherein a surface of the device comprises less than 0.01 μg of the anti-scarring agent per mm² of device surface to which the anti-scarring agent is applied.

15912. The method of item 15659 wherein a surface of the device comprises about 0.01 μg to about 1 μg of the anti-scarring agent per mm² of device surface to which the anti-scarring agent is applied.

15913. The method of item 15659 wherein a surface of the device comprises about 1 μg to about 10 μg of the anti-scarring agent per mm² of device surface to which the anti-scarring agent is applied.

15914. The method of item 15659 wherein a surface of the device comprises about 10 μg to about 250 μg of the anti-scarring agent per mm² of device surface to which the anti-scarring agent is applied.

15915. The method of item 15659 wherein a surface of the device comprises about 250 μg to about 1000 μg of the anti-scarring agent of anti-scarring agent per mm² of device surface to which the anti-scarring agent is applied.

15916. The method of item 15659 wherein a surface of the device comprises about 1000 μg to about 2500 μg of the anti-scarring agent per mm² of device surface to which the anti-scarring agent is applied.

15917. The method of item 15659 wherein the combining is performed by direct affixing the agent or the composition to the implant.

15918. The method of item 15659 wherein the combining is performed by spraying the agent or the component onto the implant.

15919. The method of item 15659 wherein the combining is performed by electrospraying the agent or the composition onto the implant.

15920. The method of item 15659 wherein the combining is performed by dipping the implant into a solution comprising the agent or the composition.

15921. The method of item 15659 wherein the combining is performed by covalently attaching the agent or the composition to the implant.

15922. The method of item 15659 wherein the combining is performed by non-covalently attaching the agent or the composition to the implant.

15923. The method of item 15659 wherein the combining is performed by coating the implant with a substance that contains the agent or the composition.

15924. The method of item 15659 wherein the combining is performed by coating the implant with a substance that absorbs the agent.

15925. The method of item 15659 wherein the combining is performed by interweaving a thread composed of, or coated with, the agent or the composition.

15926. The method of item 15659 wherein the combining is performed by covering all the implant with a sleeve that contains the agent or the composition.

15927. The method of item 15659 wherein the combining is performed by covering a portion of the implant with a sleeve that contains the agent or the composition.

15928. The method of item 15659 wherein the combining is performed by covering all the implant with a cover that contains the agent or the composition.

15929. The method of item 15659 wherein the combining is performed by covering a portion of the implant with a cover that contains the agent or the composition.

15930. The method of item 15659 wherein the combining is performed by covering all the implant with an electrospun fabric that contains the agent or the composition.

15931. The method of item 15659 wherein the combining is performed by covering a portion of the implant with an electrospun fabric that contains the agent or the composition.

15932. The method of item 15659 wherein the combining is performed by covering all the implant with a mesh that contains the agent or the composition.

15933. The method of item 15659 wherein the combining is performed by covering a portion of the implant with a mesh that contains the agent or the composition.

15934. The method of item 15659 wherein the combining is performed by constructing all the implant with the agent or the composition.

15935. The method of item 15659 wherein the combining is performed by constructing a portion of the implant with the agent or the composition.

15936. The method of item 15659 wherein the combining is performed by impregnating the implant with the agent or the composition.

15937. The method of item 15659 wherein the combining is performed by constructing all of the implant from a degradable polymer that releases the agent.

15938. The method of item 15659 wherein the combining is performed by constructing a portion of the implant from a degradable polymer that releases the agent.

15939. The method of item 15659 wherein the combining is performed by dipping the implant into a solution that comprise the agent and an inert solvent for the implant.

15940. The method of item 15659 wherein the combining is performed by dipping the implant into a solution that comprises the agent and a solvent that will swill the implant.

15941. The method of item 15659 wherein the combining is performed by dipping the implant into a solution that comprises the agent and a solvent that will dissolve the implant.

15942. The method of item 15659 wherein the combining is performed by dipping the implant into a solution that comprises the agent, a polymer and an inert solvent for the implant.

15943. The method of item 15659 wherein the combining is performed by dipping the implant into a solution that comprises the agent, a polymer and a solvent that will swill the implant.

15944. The method of item 15659 wherein the combining is performed by dipping the implant into a solution that comprises the agent, a polymer and a solvent that will dissolve the implant.

15945. The method of item 15659 wherein the combining is performed by spraying the implant into a solution that comprises the agent and an inert solvent for the implant.

15946. The method of item 15659 wherein the combining is performed by spraying the implant into a solution that comprises the agent and a solvent that will swill the implant.

15947. The method of item 15659 wherein the combining is performed by spraying the implant into a solution that comprises the agent and a solvent that will dissolve the implant.

15948. The method of item 15659 wherein the combining is performed by spraying the implant into a solution that comprises the agent, a polymer and an inert solvent for the implant.

15949. The method of item 15659 wherein the combining is performed by spraying the implant into a solution that comprises the agent, a polymer and a solvent that will swill the implant.

15950. The method of item 15659 wherein the combining is performed by spraying the implant into a solution that comprises the agent, a polymer and a solvent that will dissolve the implant.

15951. A method of making a medical device comprising: combining a tympanostomy tube implant and an anti-scarring agent or a composition comprising an anti-scarring agent, wherein the agent inhibits scarring between the device and a host into which the device is implanted.

15952. The method of item 15951 wherein the agent inhibits cell regeneration.

15953. The method of item 15951 wherein the agent inhibits angiogenesis.

15954. The method of item 15951 wherein the agent inhibits fibroblast migration.

15955. The method of item 15951 wherein the agent inhibits fibroblast proliferation.

15956. The method of item 15951 wherein the agent inhibits deposition of extracellular matrix.

15957. The method of item 15951 wherein the agent inhibits tissue remodeling.

15958. The method of item 15951 wherein the agent is an angiogenesis inhibitor.

15959. The method of item 15951 wherein the agent is a 5-lipoxygenase inhibitor or antagonist.

15960. The method of item 15951 wherein the agent is a chemokine receptor antagonist.

15961. The method of item 15951 wherein the agent is a cell cycle inhibitor.

15962. The method of item 15951 wherein the agent is a taxane.

15963. The method of item 15951 wherein the agent is an anti-microtubule agent.

15964. The method of item 15951 wherein the agent is paclitaxel.

15965. The method of item 15951 wherein the agent is not paclitaxel.

15966. The method of item 15951 wherein the agent is an analogue or derivative of paclitaxel.

15967. The method of item 15951 wherein the agent is a vinca alkaloid.

15968. The method of item 15951 wherein the agent is camptothecin or an analogue or derivative thereof.

15969. The method of item 15951 wherein the agent is a podophyllotoxin.

15970. The method of item 15951 wherein the agent is a podophyllotoxin, wherein the podophyllotoxin is etoposide or an analogue or derivative thereof.

15971. The method of item 15951 wherein the agent is an anthracycline.

15972. The method of item 15951 wherein the agent is an anthracycline, wherein the anthracycline is doxorubicin or an analogue or derivative thereof.

15973. The method of item 15951 wherein the agent is an anthracycline, wherein the anthracycline is mitoxantrone or an analogue or derivative thereof.

15974. The method of item 15951 wherein the agent is a platinum compound.

15975. The method of item 15951 wherein the agent is a nitrosourea.

15976. The method of item 15951 wherein the agent is a nitroimidazole.

15977. The method of item 15951 wherein the agent is a folic acid antagonist.

15978. The method of item 15951 wherein the agent is a cytidine analogue.

15979. The method of item 15951 wherein the agent is a pyrimidine analogue.

15980. The method of item 15951 wherein the agent is a fluoropyrimidine analogue.

15981. The method of item 15951 wherein the agent is a purine analogue.

15982. The method of item 15951 wherein the agent is a nitrogen mustard or an analogue or derivative thereof.

15983. The method of item 15951 wherein the agent is a hydroxyurea.

15984. The method of item 15951 wherein the agent is a mytomicin or an analogue or derivative thereof.

15985. The method of item 15951 wherein the agent is an alkyl sulfonate.

15986. The method of item 15951 wherein the agent is a benzamide or an analogue or derivative thereof.

15987. The method of item 15951 wherein the agent is a nicotinamide or an analogue or derivative thereof.

15988. The method of item 15951 wherein the agent is a halogenated sugar or an analogue or derivative thereof.

15989. The method of item 15951 wherein the agent is a DNA alkylating agent.

15990. The method of item 15951 wherein the agent is an anti-microtubule agent.

15991. The method of item 15951 wherein the agent is a topoisomerase inhibitor.

15992. The method of item 15951 wherein the agent is a DNA cleaving agent.

15993. The method of item 15951 wherein the agent is an antimetabolite.

15994. The method of item 15951 wherein the agent inhibits adenosine deaminase.

15995. The method of item 15951 wherein the agent inhibits purine ring synthesis.

15996. The method of item 15951 wherein the agent is a nucleotide interconversion inhibitor.

15997. The method of item 15951 wherein the agent inhibits dihydrofolate reduction.

15998. The method of item 15951 wherein the agent blocks thymidine monophosphate.

15999. The method of item 15951 wherein the agent causes DNA damage.

16000. The method of item 15951 wherein the agent is a DNA intercalation agent.

16001. The method of item 15951 wherein the agent is a RNA synthesis inhibitor.

16002. The method of item 15951 wherein the agent is a pyrimidine synthesis inhibitor.

16003. The method of item 15951 wherein the agent inhibits ribonucleotide synthesis or function.

16004. The method of item 15951 wherein the agent inhibits thymidine monophosphate synthesis or function.

16005. The method of item 15951 wherein the agent inhibits DNA synthesis.

16006. The method of item 15951 wherein the agent causes DNA adduct formation.

16007. The method of item 15951 wherein the agent inhibits protein synthesis.

16008. The method of item 15951 wherein the agent inhibits microtubule function.

16009. The method of item 15951 wherein the agent is a cyclin dependent protein kinase inhibitor.

16010. The method of item 15951 wherein the agent is an epidermal growth factor kinase inhibitor.

16011. The method of item 15951 wherein the agent is an elastase inhibitor.

16012. The method of item 15951 wherein the agent is a factor Xa inhibitor.

16013. The method of item 15951 wherein the agent is a farnesyltransferase inhibitor.

16014. The method of item 15951 wherein the agent is a fibrinogen antagonist.

16015. The method of item 15951 wherein the agent is a guanylate cyclase stimulant.

16016. The method of item 15951 wherein the agent is a heat shock protein 90 antagonist.

16017. The method of item 15951 wherein the agent is a heat shock protein 90 antagonist, wherein the heat shock protein 90 antagonist is geldanamycin or an analogue or derivative thereof.

16018. The method of item 15951 wherein the agent is a guanylate cyclase stimulant.

16019. The method of item 15951 wherein the agent is a HMGCoA reductase inhibitor.

16020. The method of item 15951 wherein the agent is a HMGCoA reductase inhibitor, wherein the HMGCoA reductase inhibitor is simvastatin or an analogue or derivative thereof.

16021. The method of item 15951 wherein the agent is a hydroorotate dehydrogenase inhibitor.

16022. The method of item 15951 wherein the agent is an IKK2 inhibitor.

16023. The method of item 15951 wherein the agent is an IL-1 antagonist.

16024. The method of item 15951 wherein the agent is an ICE antagonist.

16025. The method of item 15951 wherein the agent is an IRAK antagonist.

16026. The method of item 15951 wherein the agent is an IL-4 agonist.

16027. The method of item 15951 wherein the agent is an immunomodulatory agent.

16028. The method of item 15951 wherein the agent is sirolimus or an analogue or derivative thereof.

16029. The method of item 15951 wherein the agent is not sirolimus.

16030. The method of item 15951 wherein the agent is everolimus or an analogue or derivative thereof.

16031. The method of item 15951 wherein the agent is tacrolimus or an analogue or derivative thereof.

16032. The method of item 15951 wherein the agent is not tacrolimus.

16033. The method of item 15951 wherein the agent is biolmus or an analogue or derivative thereof.

16034. The method of item 15951 wherein the agent is tresperimus or an analogue or derivative thereof.

16035. The method of item 15951 wherein the agent is auranofin or an analogue or derivative thereof.

16036. The method of item 15951 wherein the agent is 27-0-demethylrapamycin or an analogue or derivative thereof.

16037. The method of item 15951 wherein the agent is gusperimus or an analogue or derivative thereof.

16038. The method of item 15951 wherein the agent is pimecrolimus or an analogue or derivative thereof.

16039. The method of item 15951 wherein the agent is ABT-578 or an analogue or derivative thereof.

16040. The method of item 15951 wherein the agent is an inosine monophosphate dehydrogenase (IMPDH) inhibitor.

16041. The method of item 15951 wherein the agent is an IMPDH inhibitor, wherein the IMPDH inhibitor is mycophenolic acid or an analogue or derivative thereof.

16042. The method of item 15951 wherein the agent is an IMPDH inhibitor, wherein the IMPDH inhibitor is 1-alpha-25 dihydroxy vitamin D₃ or an analogue or derivative thereof.

16043. The method of item 15951 wherein the agent is a leukotriene inhibitor.

16044. The method of item 15951 wherein the agent is a MCP-1 antagonist.

16045. The method of item 15951 wherein the agent is a MMP inhibitor.

16046. The method of item 15951 wherein the agent is an NF kappa B inhibitor.

16047. The method of item 15951 wherein the agent is an NF kappa B inhibitor, wherein the NF kappa B inhibitor is Bay 11-7082.

16048. The method of item 15951 wherein the agent is an NO agonist.

16049. The method of item 15951 wherein the agent is a p38 MAP kinase inhibitor.

16050. The method of item 15951 wherein the agent is a p38 MAP kinase inhibitor, wherein the p38 MAP kinase inhibitor is SB 202190.

16051. The method of item 15951 wherein the agent is a phosphodiesterase inhibitor.

16052. The method of item 15951 wherein the agent is a TGF beta inhibitor.

16053. The method of item 15951 wherein the agent is a thromboxane A2 antagonist.

16054. The method of item 15951 wherein the agent is a TNFa antagonist.

16055. The method of item 15951 wherein the agent is a TACE inhibitor.

16056. The method of item 15951 wherein the agent is a tyrosine kinase inhibitor.

16057. The method of item 15951 wherein the agent is a vitronectin inhibitor.

16058. The method of item 15951 wherein the agent is a fibroblast growth factor inhibitor.

16059. The method of item 15951 wherein the agent is a protein kinase inhibitor.

16060. The method of item 15951 wherein the agent is a PDGF receptor kinase inhibitor.

16061. The method of item 15951 wherein the agent is an endothelial growth factor receptor kinase inhibitor.

16062. The method of item 15951 wherein the agent is a retinoic acid receptor antagonist.

16063. The method of item 15951 wherein the agent is a platelet derived growth factor receptor kinase inhibitor.

16064. The method of item 15951 wherein the agent is a fibronogin antagonist.

16065. The method of item 15951 wherein the agent is an antimycotic agent.

16066. The method of item 15951 wherein the agent is an antimycotic agent, wherein the antimycotic agent is sulconizole.

16067. The method of item 15951 wherein the agent is a bisphosphonate.

16068. The method of item 15951 wherein the agent is a phospholipase A1 inhibitor.

16069. The method of item 15951 wherein the agent is a histamine H1/H2/H3 receptor antagonist.

16070. The method of item 15951 wherein the agent is a macrolide antibiotic.

16071. The method of item 15951 wherein the agent is a GPIIb/IIIa receptor antagonist.

16072. The method of item 15951 wherein the agent is an endothelin receptor antagonist.

16073. The method of item 15951 wherein the agent is a peroxisome proliferator-activated receptor agonist.

16074. The method of item 15951 wherein the agent is an estrogen receptor agent.

16075. The method of item 15951 wherein the agent is a somastostatin analogue.

16076. The method of item 15951 wherein the agent is a neurokinin 1 antagonist.

16077. The method of item 15951 wherein the agent is a neurokinin 3 antagonist.

16078. The method of item 15951 wherein the agent is a VLA-4 antagonist.

16079. The method of item 15951 wherein the agent is an osteoclast inhibitor.

16080. The method of item 15951 wherein the agent is a DNA topoisomerase ATP hydrolyzing inhibitor.

16081. The method of item 15951 wherein the agent is an angiotensin I converting enzyme inhibitor.

16082. The method of item 15951 wherein the agent is an angiotensin II antagonist.

16083. The method of item 15951 wherein the agent is an enkephalinase inhibitor.

16084. The method of item 15951 wherein the agent is a peroxisome proliferator-activated receptor gamma agonist insulin sensitizer.

16085. The method of item 15951 wherein the agent is a protein kinase C inhibitor.

16086. The method of item 15951 wherein the agent is a ROCK (rho-associated kinase) inhibitor.

16087. The method of item 15951 wherein the agent is a CXCR3 inhibitor.

16088. The method of item 15951 wherein the agent is an Itk inhibitor.

16089. The method of item 15951 wherein the agent is a cytosolic phospholipase A₂-alpha inhibitor.

16090. The method of item 15951 wherein the agent is a PPAR agonist.

16091. The method of item 15951 wherein the agent is an immunosuppressant.

16092. The method of item 15951 wherein the agent is an Erb inhibitor.

16093. The method of item 15951 wherein the agent is an apoptosis agonist.

16094. The method of item 15951 wherein the agent is a lipocortin agonist.

16095. The method of item 15951 wherein the agent is a VCAM-1 antagonist.

16096. The method of item 15951 wherein the agent is a collagen antagonist.

16097. The method of item 15951 wherein the agent is an alpha 2 integrin antagonist.

16098. The method of item 15951 wherein the agent is a TNF alpha inhibitor.

16099. The method of item 15951 wherein the agent is a nitric oxide inhibitor.

16100. The method of item 15951 wherein the agent is a cathepsin inhibitor.

16101. The method of item 15951 wherein the agent is not an anti-inflammatory agent.

16102. The method of item 15951 wherein the agent is not a steroid.

16103. The method of item 15951 wherein the agent is not a glucocorticosteroid.

16104. The method of item 15951 wherein the agent is not dexamethasone.

16105. The method of item 15951 wherein the agent is not an anti-infective agent.

16106. The method of item 15951 wherein the agent is not an antibiotic.

16107. The method of item 15951 wherein the agent is not an anti-fungal agent.

16108. The method of item 15951, wherein the composition comprises a polymer.

16109. The method of item 15951, wherein the composition comprises a polymeric carrier.

16110. The method of item 15951 wherein the anti-scarring agent inhibits adhesion between the device and a host into which the device is implanted.

16111. The method of item 15951 wherein the device delivers the anti-scarring agent locally to tissue proximate to the device.

16112. The method of item 15951 wherein the device has a coating that comprises the anti-scarring agent.

16113. The method of item 15951, wherein the device has a coating that comprises the agent and is disposed on a surface of the implant.

16114. The method of item 15951, wherein the device has a coating that comprises the agent and directly contacts the implant.

16115. The method of item 15951, wherein the device has a coating that comprises the agent and indirectly contacts the implant.

16116. The method of item 15951, wherein the device has a coating that comprises the agent and partially covers the implant.

16117. The method of item 15951, wherein the device has a coating that comprises the agent and completely covers the implant.

16118. The method of item 15951, wherein the device has a uniform coating.

16119. The method of item 15951, wherein the device has a non-uniform coating.

16120. The method of item 15951, wherein the device has a discontinuous coating.

16121. The method of item 15951, wherein the device has a patterned coating.

16122. The method of item 15951, wherein the device has a coating with a thickness of 100 μm or less.

16123. The method of item 15951, wherein the device has a coating with a thickness of 10 μm or less.

16124. The method of item 15951, wherein the device has a coating, and the coating adheres to the surface of the implant upon deployment of the implant.

16125. The method of item 15951, wherein the device has a coating, and wherein the coating is stable at room temperature for a period of 1 year.

16126. The method of item 15951, wherein the device has a coating, and wherein the anti-scarring agent is present in the coating in an amount ranging between about 0.0001% to about 1% by weight.

16127. The method of item 15951, wherein the device has a coating, and wherein the anti-scarring agent is present in the coating in an amount ranging between about 1% to about 10% by weight.

16128. The method of item 15951, wherein the device has a coating, and wherein the anti-scarring agent is present in the coating in an amount ranging between about 10% to about 25% by weight.

16129. The method of item 15951, wherein the device has a coating, and wherein the anti-scarring agent is present in the coating in an amount ranging between about 25% to about 70% by weight.

16130. The method of item 15951, wherein the device has a coating, and wherein the coating further comprises a polymer.

16131. The method of item 15951, wherein the device has a first coating having a first composition and a second coating having a second composition.

16132. The method of item 15951, wherein the device has a first coating having a first composition and a second coating having a second composition, wherein the first composition and the second composition are different.

16133. The method of item 15951, wherein the composition comprises a polymer.

16134. The method of item 15951, wherein the composition comprises a polymeric carrier.

16135. The method of item 15951, wherein the composition comprises a polymeric carrier, and wherein the polymeric carrier comprises a copolymer.

16136. The method of item 15951, wherein the composition comprises a polymeric carrier, and wherein the polymeric carrier comprises a block copolymer.

16137. The method of item 15951, wherein the composition comprises a polymeric carrier, and wherein the polymeric carrier comprises a random copolymer.

16138. The method of item 15951, wherein the composition comprises a polymeric carrier, and wherein the polymeric carrier comprises a biodegradable polymer.

16139. The method of item 15951, wherein the composition comprises a polymeric carrier, and wherein the polymeric carrier comprises a non-biodegradable polymer.

16140. The method of item 15951, wherein the composition comprises a polymeric carrier, and wherein the polymeric carrier comprises a hydrophilic polymer.

16141. The method of item 15951, wherein the composition comprises a polymeric carrier, and wherein the polymeric carrier comprises a hydrophobic polymer.

16142. The method of item 15951, wherein the composition comprises a polymeric carrier, and wherein the polymeric carrier comprises a polymer having hydrophilic domains.

16143. The method of item 15951, wherein the composition comprises a polymeric carrier, and wherein the polymeric carrier comprises a polymer having hydrophobic domains.

16144. The method of item 15951, wherein the composition comprises a polymeric carrier, and wherein the polymeric carrier comprises a non-conductive polymer.

16145. The method of item 15951, wherein the composition comprises a polymeric carrier, and wherein the polymeric carrier comprises an elastomer.

16146. The method of item 15951, wherein the composition comprises a polymeric carrier, and wherein the polymeric carrier comprises a hydrogel.

16147. The method of item 15951, wherein the composition comprises a polymeric carrier, and wherein the polymeric carrier comprises a silicone polymer.

16148. The method of item 15951, wherein the composition comprises a polymeric carrier, and wherein the polymeric carrier comprises a hydrocarbon polymer.

16149. The method of item 15951, wherein the composition comprises a polymeric carrier, and wherein the polymeric carrier comprises a styrene-derived polymer.

16150. The method of item 15951, wherein the composition comprises a polymeric carrier, and wherein the polymeric carrier comprises a butadiene polymer.

16151. The method of item 15951, wherein the composition comprises a polymeric carrier, and wherein the polymeric carrier comprises a macromer.

16152. The method of item 15951, wherein the composition comprises a polymeric carrier, and wherein the polymeric carrier comprises a poly(ethylene glycol)polymer.

16153. The method of item 15951 wherein the composition comprises a polymeric carrier, and wherein the polymeric carrier comprises an amorphous polymer.

16154. The method of item 15951, wherein the device comprises a lubricious coating.

16155. The method of item 15951 wherein the anti-scarring agent is located within pores or holes of the device.

16156. The method of item 15951 wherein the anti-scarring agent is located within a channel, lumen, or divet of the device.

16157. The method of item 15951, wherein the device comprises a second pharmaceutically active agent.

16158. The method of item 15951 wherein the device comprises an anti-inflammatory agent.

16159. The method of item 15951 wherein the device comprises an agent that inhibits infection.

16160. The method of item 15951 wherein the device comprises an agent that inhibits infection, and wherein the agent is an anthracycline.

16161. The method of item 15951 wherein the device comprises an agent that inhibits infection, and wherein the agent is doxorubicin.

16162. The method of item 15951 wherein the device comprises an agent that inhibits infection, and wherein the agent is mitoxantrone.

16163. The method of item 15951 wherein the device comprises an agent that inhibits infection, and wherein the agent is a fluoropyrimidine.

16164. The method of item 15951 wherein the device comprises an agent that inhibits infection, and wherein the agent is 5-fluorouracil (5-FU).

16165. The method of item 15951 wherein the device comprises an agent that inhibits infection, and wherein the agent is a folic acid antagonist.

16166. The method of item 15951 wherein the device comprises an agent that inhibits infection, and wherein the agent is methotrexate.

16167. The method of item 15951 wherein the device comprises an agent that inhibits infection, and wherein the agent is a podophylotoxin.

16168. The method of item 15951 wherein the device comprises an agent that inhibits infection, and wherein the agent is etoposide.

16169. The method of item 15951 wherein the device comprises an agent that inhibits infection, and wherein the agent is a camptothecin.

16170. The method of item 15951 wherein the device comprises an agent that inhibits infection, and wherein the agent is a hydroxyurea.

16171. The method of item 15951 wherein the device comprises an agent that inhibits infection, and wherein the agent is a platinum complex.

16172. The method of item 15951 wherein the device comprises an agent that inhibits infection, and wherein the agent is cisplatin.

16173. The method of item 15951, further comprising an anti-thrombotic agent.

16174. The method of item 15951 wherein the device comprises a visualization agent.

16175. The method of item 15951 wherein the device comprises a visualization agent, wherein the visualization agent is a radiopaque material, and wherein the radiopaque material comprises a metal, a halogenated compound, or a barium containing compound.

16176. The method of item 15951 wherein the device comprises a visualization agent, wherein the visualization agent is a radiopaque material, and wherein the radiopaque material comprises barium, tantalum, or technetium.

16177. The method of item 15951 wherein the device comprises a visualization agent, and wherein the visualization agent is a MRI responsive material.

16178. The method of item 15951 wherein the device comprises a visualization agent, and wherein the visualization agent comprises a gadolinium chelate.

16179. The method of item 15951 wherein the device comprises a visualization agent, and wherein the visualization agent comprises iron, magnesium, manganese, copper, or chromium.

16180. The method of item 15951 wherein the device comprises a visualization agent, and wherein the visualization agent comprises an iron oxide compound.

16181. The method of item 15951 wherein the device comprises a visualization agent, and wherein the visualization agent comprises a dye, pigment, or colorant.

16182. The method of item 15951 wherein the device comprises an echogenic material.

16183. The method of item 15951 wherein the device comprises an echogenic material, and wherein the echogenic material is in the form of a coating.

16184. The method of item 15951 wherein the device is sterile.

16185. The method of item 15951 wherein the anti-scarring agent is released into tissue in the vicinity of the device after deployment of the device.

16186. The method of item 15951 wherein the anti-scarring agent is released into tissue in the vicinity of the device after deployment of the device, and wherein the tissue is connective tissue.

16187. The method of item 15951 wherein the anti-scarring agent is released into tissue in the vicinity of the device after deployment of the device, and wherein the tissue is muscle tissue.

16188. The method of item 15951 wherein the anti-scarring agent is released into tissue in the vicinity of the device after deployment of the device, and wherein the tissue is nerve tissue.

16189. The method of item 15951 wherein the anti-scarring agent is released into tissue in the vicinity of the device after deployment of the device, and wherein the tissue is epithelium tissue.

16190. The method of item 15951 wherein the anti-scarring agent is released in effective concentrations from the device over a period ranging from the time of deployment of the device to about 1 year.

16191. The method of item 15951 wherein the anti-scarring agent is released in effective concentrations from the device over a period ranging from about 1 month to 6 months.

16192. The method of item 15951 wherein the anti-scarring agent is released in effective concentrations from the device over a period ranging from about 1-90 days.

16193. The method of item 15951 wherein the anti-scarring agent is released in effective concentrations from the device at a constant rate.

16194. The method of item 15951 wherein the anti-scarring agent is released in effective concentrations from the device at an increasing rate.

16195. The method of item 15951 wherein the anti-scarring agent is released in effective concentrations from the device at a decreasing rate.

16196. The method of item 15951 wherein the anti-scarring agent is released in effective concentrations from the composition comprising the anti-scarring agent by diffusion over a period ranging from the time of deployment of the device to about 90 days.

16197. The method of item 15951 wherein the anti-scarring agent is released in effective concentrations from the composition comprising the anti-scarring agent by erosion of the composition over a period ranging from the time of deployment of the device to about 90 days.

16198. The method of item 15951 wherein the device comprises about 0.01 μg to about 10 μg of the anti-scarring agent.

16199. The method of item 15951 wherein the device comprises about 10 μg to about 10 mg of the anti-scarring agent.

16200. The method of item 15951 wherein the device comprises about 10 mg to about 250 mg of the anti-scarring agent.

16201. The method of item 15951 wherein the device comprises about 250 mg to about 1000 mg of the anti-scarring agent.

16202. The method of item 15951 wherein the device comprises about 1000 mg to about 2500 mg of the anti-scarring agent.

16203. The method of item 15951 wherein a surface of the device comprises less than 0.01 μg of the anti-scarring agent per mm² of device surface to which the anti-scarring agent is applied.

16204. The method of item 15951 wherein a surface of the device comprises about 0.01 μg to about 1 μg of the anti-scarring agent per mm² of device surface to which the anti-scarring agent is applied.

16205. The method of item 15951 wherein a surface of the device comprises about 1 μg to about 10 μg of the anti-scarring agent per mm² of device surface to which the anti-scarring agent is applied.

16206. The method of item 15951 wherein a surface of the device comprises about 10 μg to about 250 μg of the anti-scarring agent per mm² of device surface to which the anti-scarring agent is applied.

16207. The method of item 15951 wherein a surface of the device comprises about 250 μg to about 1000 μg of the anti-scarring agent of anti-scarring agent per mm² of device surface to which the anti-scarring agent is applied.

16208. The method of item 15951 wherein a surface of the device comprises about 1000 μg to about 2500 μg of the anti-scarring agent per mm² of device surface to which the anti-scarring agent is applied.

16209. The method of item 15951 wherein the combining is performed by direct affixing the agent or the composition to the implant.

16210. The method of item 15951 wherein the combining is performed by spraying the agent or the component onto the implant.

16211. The method of item 15951 wherein the combining is performed by electrospraying the agent or the composition onto the implant.

16212. The method of item 15951 wherein the combining is performed by dipping the implant into a solution comprising the agent or the composition.

16213. The method of item 15951 wherein the combining is performed by covalently attaching the agent or the composition to the implant.

16214. The method of item 15951 wherein the combining is performed by non-covalently attaching the agent or the composition to the implant.

16215. The method of item 15951 wherein the combining is performed by coating the implant with a substance that contains the agent or the composition.

16216. The method of item 15951 wherein the combining is performed by coating the implant with a substance that absorbs the agent.

16217. The method of item 15951 wherein the combining is performed by interweaving a thread composed of, or coated with, the agent or the composition.

16218. The method of item 15951 wherein the combining is performed by covering all the implant with a sleeve that contains the agent or the composition.

16219. The method of item 15951 wherein the combining is performed by covering a portion of the implant with a sleeve that contains the agent or the composition.

16220. The method of item 15951 wherein the combining is performed by covering all the implant with a cover that contains the agent or the composition.

16221. The method of item 15951 wherein the combining is performed by covering a portion of the implant with a cover that contains the agent or the composition.

16222. The method of item 15951 wherein the combining is performed by covering all the implant with an electrospun fabric that contains the agent or the composition.

16223. The method of item 15951 wherein the combining is performed by covering a portion of the implant with an electrospun fabric that contains the agent or the composition.

16224. The method of item 15951 wherein the combining is performed by covering all the implant with a mesh that contains the agent or the composition.

16225. The method of item 15951 wherein the combining is performed by covering a portion of the implant with a mesh that contains the agent or the composition.

16226. The method of item 15951 wherein the combining is performed by constructing all the implant with the agent or the composition.

16227. The method of item 15951 wherein the combining is performed by constructing a portion of the implant with the agent or the composition.

16228. The method of item 15951 wherein the combining is performed by impregnating the implant with the agent or the composition.

16229. The method of item 15951 wherein the combining is performed by constructing all of the implant from a degradable polymer that releases the agent.

16230. The method of item 15951 wherein the combining is performed by constructing a portion of the implant from a degradable polymer that releases the agent.

16231. The method of item 15951 wherein the combining is performed by dipping the implant into a solution that comprise the agent and an inert solvent for the implant.

16232. The method of item 15951 wherein the combining is performed by dipping the implant into a solution that comprises the agent and a solvent that will swill the implant.

16233. The method of item 15951 wherein the combining is performed by dipping the implant into a solution that comprises the agent and a solvent that will dissolve the implant.

16234. The method of item 15951 wherein the combining is performed by dipping the implant into a solution that comprises the agent, a polymer and an inert solvent for the implant.

16235. The method of item 15951 wherein the combining is performed by dipping the implant into a solution that comprises the agent, a polymer and a solvent that will swill the implant.

16236. The method of item 15951 wherein the combining is performed by dipping the implant into a solution that comprises the agent, a polymer and a solvent that will dissolve the implant.

16237. The method of item 15951 wherein the combining is performed by spraying the implant into a solution that comprises the agent and an inert solvent for the implant.

16238. The method of item 15951 wherein the combining is performed by spraying the implant into a solution that comprises the agent and a solvent that will swill the implant.

16239. The method of item 15951 wherein the combining is performed by spraying the implant into a solution that comprises the agent and a solvent that will dissolve the implant.

16240. The method of item 15951 wherein the combining is performed by spraying the implant into a solution that comprises the agent, a polymer and an inert solvent for the implant.

16241. The method of item 15951 wherein the combining is performed by spraying the implant into a solution that comprises the agent, a polymer and a solvent that will swill the implant.

16242. The method of item 15951 wherein the combining is performed by spraying the implant into a solution that comprises the agent, a polymer and a solvent that will dissolve the implant.

16243. A method of making a medical device comprising: combining an implant that provides a surgical adhesion barrier and an anti-scarring agent or a composition comprising an anti-scarring agent, wherein the agent inhibits scarring between the device and a host into which the device is implanted.

16244. The method of item 16243 wherein the agent inhibits cell regeneration.

16245. The method of item 16243 wherein the agent inhibits angiogenesis.

16246. The method of item 16243 wherein the agent inhibits fibroblast migration.

16247. The method of item 16243 wherein the agent inhibits fibroblast proliferation.

16248. The method of item 16243 wherein the agent inhibits deposition of extracellular matrix.

16249. The method of item 16243 wherein the agent inhibits tissue remodeling.

16250. The method of item 16243 wherein the agent is an angiogenesis inhibitor.

16251. The method of item 16243 wherein the agent is a 5-lipoxygenase inhibitor or antagonist.

16252. The method of item 16243 wherein the agent is a chemokine receptor antagonist.

16253. The method of item 16243 wherein the agent is a cell cycle inhibitor.

16254. The method of item 16243 wherein the agent is a taxane.

16255. The method of item 16243 wherein the agent is an anti-microtubule agent.

16256. The method of item 16243 wherein the agent is paclitaxel.

16257. The method of item 16243 wherein the agent is not paclitaxel.

16258. The method of item 16243 wherein the agent is an analogue or derivative of paclitaxel.

16259. The method of item 16243 wherein the agent is a vinca alkaloid.

16260. The method of item 16243 wherein the agent is camptothecin or an analogue or derivative thereof.

16261. The method of item 16243 wherein the agent is a podophyllotoxin.

16262. The method of item 16243 wherein the agent is a podophyllotoxin, wherein the podophyllotoxin is etoposide or an analogue or derivative thereof.

16263. The method of item 16243 wherein the agent is an anthracycline.

16264. The method of item 16243 wherein the agent is an anthracycline, wherein the anthracycline is doxorubicin or an analogue or derivative thereof.

16265. The method of item 16243 wherein the agent is an anthracycline, wherein the anthracycline is mitoxantrone or an analogue or derivative thereof.

16266. The method of item 16243 wherein the agent is a platinum compound.

16267. The method of item 16243 wherein the agent is a nitrosourea.

16268. The method of item 16243 wherein the agent is a nitroimidazole.

16269. The method of item 16243 wherein the agent is a folic acid antagonist.

16270. The method of item 16243 wherein the agent is a cytidine analogue.

16271. The method of item 16243 wherein the agent is a pyrimidine analogue.

16272. The method of item 16243 wherein the agent is a fluoropyrimidine analogue.

16273. The method of item 16243 wherein the agent is a purine analogue.

16274. The method of item 16243 wherein the agent is a nitrogen mustard or an analogue or derivative thereof.

16275. The method of item 16243 wherein the agent is a hydroxyurea.

16276. The method of item 16243 wherein the agent is a mytomicin or an analogue or derivative thereof.

16277. The method of item 16243 wherein the agent is an alkyl sulfonate.

16278. The method of item 16243 wherein the agent is a benzamide or an analogue or derivative thereof.

16279. The method of item 16243 wherein the agent is a nicotinamide or an analogue or derivative thereof.

16280. The method of item 16243 wherein the agent is a halogenated sugar or an analogue or derivative thereof.

16281. The method of item 16243 wherein the agent is a DNA alkylating agent.

16282. The method of item 16243 wherein the agent is an anti-microtubule agent.

16283. The method of item 16243 wherein the agent is a topoisomerase inhibitor.

16284. The method of item 16243 wherein the agent is a DNA cleaving agent.

16285. The method of item 16243 wherein the agent is an antimetabolite.

16286. The method of item 16243 wherein the agent inhibits adenosine deaminase.

16287. The method of item 16243 wherein the agent inhibits purine ring synthesis.

16288. The method of item 16243 wherein the agent is a nucleotide interconversion inhibitor.

16289. The method of item 16243 wherein the agent inhibits dihydrofolate reduction.

16290. The method of item 16243 wherein the agent blocks thymidine monophosphate.

16291. The method of item 16243 wherein the agent causes DNA damage.

16292. The method of item 16243 wherein the agent is a DNA intercalation agent.

16293. The method of item 16243 wherein the agent is a RNA synthesis inhibitor.

16294. The method of item 16243 wherein the agent is a pyrimidine synthesis inhibitor.

16295. The method of item 16243 wherein the agent inhibits ribonucleotide synthesis or function.

16296. The method of item 16243 wherein the agent inhibits thymidine monophosphate synthesis or function.

16297. The method of item 16243 wherein the agent inhibits DNA synthesis.

16298. The method of item 16243 wherein the agent causes DNA adduct formation.

16299. The method of item 16243 wherein the agent inhibits protein synthesis.

16300. The method of item 16243 wherein the agent inhibits microtubule function.

16301. The method of item 16243 wherein the agent is a cyclin dependent protein kinase inhibitor.

16302. The method of item 16243 wherein the agent is an epidermal growth factor kinase inhibitor.

16303. The method of item 16243 wherein the agent is an elastase inhibitor.

16304. The method of item 16243 wherein the agent is a factor Xa inhibitor.

16305. The method of item 16243 wherein the agent is a farnesyltransferase inhibitor.

16306. The method of item 16243 wherein the agent is a fibrinogen antagonist.

16307. The method of item 16243 wherein the agent is a guanylate cyclase stimulant.

16308. The method of item 16243 wherein the agent is a heat shock protein 90 antagonist.

16309. The method of item 16243 wherein the agent is a heat shock protein 90 antagonist, wherein the heat shock protein 90 antagonist is geldanamycin or an analogue or derivative thereof.

16310. The method of item 16243 wherein the agent is a guanylate cyclase stimulant.

16311. The method of item 16243 wherein the agent is a HMGCoA reductase inhibitor.

16312. The method of item 16243 wherein the agent is a HMGCoA reductase inhibitor, wherein the HMGCoA reductase inhibitor is simvastatin or an analogue or derivative thereof.

16313. The method of item 16243 wherein the agent is a hydroorotate dehydrogenase inhibitor.

16314. The method of item 16243 wherein the agent is an IKK2 inhibitor.

16315. The method of item 16243 wherein the agent is an IL-1 antagonist.

16316. The method of item 16243 wherein the agent is an ICE antagonist.

16317. The method of item 16243 wherein the agent is an IRAK antagonist.

16318. The method of item 16243 wherein the agent is an IL-4 agonist.

16319. The method of item 16243 wherein the agent is an immunomodulatory agent.

16320. The method of item 16243 wherein the agent is sirolimus or an analogue or derivative thereof.

16321. The method of item 16243 wherein the agent is not sirolimus.

16322. The method of item 16243 wherein the agent is everolimus or an analogue or derivative thereof.

16323. The method of item 16243 wherein the agent is tacrolimus or an analogue or derivative thereof.

16324. The method of item 16243 wherein the agent is not tacrolimus.

16325. The method of item 16243 wherein the agent is biolmus or an analogue or derivative thereof.

16326. The method of item 16243 wherein the agent is tresperimus or an analogue or derivative thereof.

16327. The method of item 16243 wherein the agent is auranofin or an analogue or derivative thereof.

16328. The method of item 16243 wherein the agent is 27-0-demethylrapamycin or an analogue or derivative thereof.

16329. The method of item 16243 wherein the agent is gusperimus or an analogue or derivative thereof.

16330. The method of item 16243 wherein the agent is pimecrolimus or an analogue or derivative thereof.

16331. The method of item 16243 wherein the agent is ABT-578 or an analogue or derivative thereof.

16332. The method of item 16243 wherein the agent is an inosine monophosphate dehydrogenase (IMPDH) inhibitor.

16333. The method of item 16243 wherein the agent is an IMPDH inhibitor, wherein the IMPDH inhibitor is mycophenolic acid or an analogue or derivative thereof.

16334. The method of item 16243 wherein the agent is an IMPDH inhibitor, wherein the IMPDH inhibitor is 1-alpha-25 dihydroxy vitamin D₃ or an analogue or derivative thereof.

16335. The method of item 16243 wherein the agent is a leukotriene inhibitor.

16336. The method of item 16243 wherein the agent is a MCP-1 antagonist.

16337. The method of item 16243 wherein the agent is a MMP inhibitor.

16338. The method of item 16243 wherein the agent is an NF kappa B inhibitor.

16339. The method of item 16243 wherein the agent is an NF kappa B inhibitor, wherein the NF kappa B inhibitor is Bay 11-7082.

16340. The method of item 16243 wherein the agent is an NO agonist.

16341. The method of item 16243 wherein the agent is a p38 MAP kinase inhibitor.

16342. The method of item 16243 wherein the agent is a p38 MAP kinase inhibitor, wherein the p38 MAP kinase inhibitor is SB 202190.

16343. The method of item 16243 wherein the agent is a phosphodiesterase inhibitor.

16344. The method of item 16243 wherein the agent is a TGF beta inhibitor.

16345. The method of item 16243 wherein the agent is a thromboxane A2 antagonist.

16346. The method of item 16243 wherein the agent is a TNFa antagonist.

16347. The method of item 16243 wherein the agent is a TACE inhibitor.

16348. The method of item 16243 wherein the agent is a tyrosine kinase inhibitor.

16349. The method of item 16243 wherein the agent is a vitronectin inhibitor.

16350. The method of item 16243 wherein the agent is a fibroblast growth factor inhibitor.

16351. The method of item 16243 wherein the agent is a protein kinase inhibitor.

16352. The method of item 16243 wherein the agent is a PDGF receptor kinase inhibitor.

16353. The method of item 16243 wherein the agent is an endothelial growth factor receptor kinase inhibitor.

16354. The method of item 16243 wherein the agent is a retinoic acid receptor antagonist.

16355. The method of item 16243 wherein the agent is a platelet derived growth factor receptor kinase inhibitor.

16356. The method of item 16243 wherein the agent is a fibronogin antagonist.

16357. The method of item 16243 wherein the agent is an antimycotic agent.

16358. The method of item 16243 wherein the agent is an antimycotic agent, wherein the antimycotic agent is sulconizole.

16359. The method of item 16243 wherein the agent is a bisphosphonate.

16360. The method of item 16243 wherein the agent is a phospholipase A1 inhibitor.

16361. The method of item 16243 wherein the agent is a histamine H1/H2/H3 receptor antagonist.

16362. The method of item 16243 wherein the agent is a macrolide antibiotic.

16363. The method of item 16243 wherein the agent is a GPIIb/IIIa receptor antagonist.

16364. The method of item 16243 wherein the agent is an endothelin receptor antagonist.

16365. The method of item 16243 wherein the agent is a peroxisome proliferator-activated receptor agonist.

16366. The method of item 16243 wherein the agent is an estrogen receptor agent.

16367. The method of item 16243 wherein the agent is a somastostatin analogue.

16368. The method of item 16243 wherein the agent is a neurokinin 1 antagonist.

16369. The method of item 16243 wherein the agent is a neurokinin 3 antagonist.

16370. The method of item 16243 wherein the agent is a VLA-4 antagonist.

16371. The method of item 16243 wherein the agent is an osteoclast inhibitor.

16372. The method of item 16243 wherein the agent is a DNA topoisomerase ATP hydrolyzing inhibitor.

16373. The method of item 16243 wherein the agent is an angiotensin I converting enzyme inhibitor.

16374. The method of item 16243 wherein the agent is an angiotensin II antagonist.

16375. The method of item 16243 wherein the agent is an enkephalinase inhibitor.

16376. The method of item 16243 wherein the agent is a peroxisome proliferator-activated receptor gamma agonist insulin sensitizer.

16377. The method of item 16243 wherein the agent is a protein kinase C inhibitor.

16378. The method of item 16243 wherein the agent is a ROCK (rho-associated kinase) inhibitor.

16379. The method of item 16243 wherein the agent is a CXCR3 inhibitor.

16380. The method of item 16243 wherein the agent is an Itk inhibitor.

16381. The method of item 16243 wherein the agent is a cytosolic phospholipase A₂-alpha inhibitor.

16382. The method of item 16243 wherein the agent is a PPAR agonist.

16383. The method of item 16243 wherein the agent is an immunosuppressant.

16384. The method of item 16243 wherein the agent is an Erb inhibitor.

16385. The method of item 16243 wherein the agent is an apoptosis agonist.

16386. The method of item 16243 wherein the agent is a lipocortin agonist.

16387. The method of item 16243 wherein the agent is a VCAM-1 antagonist.

16388. The method of item 16243 wherein the agent is a collagen antagonist.

16389. The method of item 16243 wherein the agent is an alpha 2 integrin antagonist.

16390. The method of item 16243 wherein the agent is a TNF alpha inhibitor.

16391. The method of item 16243 wherein the agent is a nitric oxide inhibitor.

16392. The method of item 16243 wherein the agent is a cathepsin inhibitor.

16393. The method of item 16243 wherein the agent is not an anti-inflammatory agent.

16394. The method of item 16243 wherein the agent is not a steroid.

16395. The method of item 16243 wherein the agent is not a glucocorticosteroid.

16396. The method of item 16243 wherein the agent is not dexamethasone.

16397. The method of item 16243 wherein the agent is not an anti-infective agent.

16398. The method of item 16243 wherein the agent is not an antibiotic.

16399. The method of item 16243 wherein the agent is not an anti-fungal agent.

16400. The method of item 16243, wherein the composition comprises a polymer.

16401. The method of item 16243, wherein the composition comprises a polymeric carrier.

16402. The method of item 16243 wherein the anti-scarring agent inhibits adhesion between the device and a host into which the device is implanted.

16403. The method of item 16243 wherein the device delivers the anti-scarring agent locally to tissue proximate to the device.

16404. The method of item 16243 wherein the device has a coating that comprises the anti-scarring agent.

16405. The method of item 16243, wherein the device has a coating that comprises the agent and is disposed on a surface of the implant.

16406. The method of item 16243, wherein the device has a coating that comprises the agent and directly contacts the implant.

16407. The method of item 16243, wherein the device has a coating that comprises the agent and indirectly contacts the implant.

16408. The method of item 16243, wherein the device has a coating that comprises the agent and partially covers the implant.

16409. The method of item 16243, wherein the device has a coating that comprises the agent and completely covers the implant.

16410. The method of item 16243, wherein the device has a uniform coating.

16411. The method of item 16243, wherein the device has a non-uniform coating.

16412. The method of item 16243, wherein the device has a discontinuous coating.

16413. The method of item 16243, wherein the device has a patterned coating.

16414. The method of item 16243, wherein the device has a coating with a thickness of 100 μm or less.

16415. The method of item 16243, wherein the device has a coating with a thickness of 10 μm or less.

16416. The method of item 16243, wherein the device has a coating, and the coating adheres to the surface of the implant upon deployment of the implant.

16417. The method of item 16243, wherein the device has a coating, and wherein the coating is stable at room temperature for a period of 1 year.

16418. The method of item 16243, wherein the device has a coating, and wherein the anti-scarring agent is present in the coating in an amount ranging between about 0.0001% to about 1% by weight.

16419. The method of item 16243, wherein the device has a coating, and wherein the anti-scarring agent is present in the coating in an amount ranging between about 1% to about 10% by weight.

16420. The method of item 16243, wherein the device has a coating, and wherein the anti-scarring agent is present in the coating in an amount ranging between about 10% to about 25% by weight.

16421. The method of item 16243, wherein the device has a coating, and wherein the anti-scarring agent is present in the coating in an amount ranging between about 25% to about 70% by weight.

16422. The method of item 16243, wherein the device has a coating, and wherein the coating further comprises a polymer.

16423. The method of item 16243, wherein the device has a first coating having a first composition and a second coating having a second composition.

16424. The method of item 16243, wherein the device has a first coating having a first composition and a second coating having a second composition, wherein the first composition and the second composition are different.

16425. The method of item 16243, wherein the composition comprises a polymer.

16426. The method of item 16243, wherein the composition comprises a polymeric carrier.

16427. The method of item 16243, wherein the composition comprises a polymeric carrier, and wherein the polymeric carrier comprises a copolymer.

16428. The method of item 16243, wherein the composition comprises a polymeric carrier, and wherein the polymeric carrier comprises a block copolymer.

16429. The method of item 16243, wherein the composition comprises a polymeric carrier, and wherein the polymeric carrier comprises a random copolymer.

16430. The method of item 16243, wherein the composition comprises a polymeric carrier, and wherein the polymeric carrier comprises a biodegradable polymer.

16431. The method of item 16243, wherein the composition comprises a polymeric carrier, and wherein the polymeric carrier comprises a non-biodegradable polymer.

16432. The method of item 16243, wherein the composition comprises a polymeric carrier, and wherein the polymeric carrier comprises a hydrophilic polymer.

16433. The method of item 16243, wherein the composition comprises a polymeric carrier, and wherein the polymeric carrier comprises a hydrophobic polymer.

16434. The method of item 16243, wherein the composition comprises a polymeric carrier, and wherein the polymeric carrier comprises a polymer having hydrophilic domains.

16435. The method of item 16243, wherein the composition comprises a polymeric carrier, and wherein the polymeric carrier comprises a polymer having hydrophobic domains.

16436. The method of item 16243, wherein the composition comprises a polymeric carrier, and wherein the polymeric carrier comprises a non-conductive polymer.

16437. The method of item 16243, wherein the composition comprises a polymeric carrier, and wherein the polymeric carrier comprises an elastomer.

16438. The method of item 16243, wherein the composition comprises a polymeric carrier, and wherein the polymeric carrier comprises a hydrogel.

16439. The method of item 16243, wherein the composition comprises a polymeric carrier, and wherein the polymeric carrier comprises a silicone polymer.

16440. The method of item 16243, wherein the composition comprises a polymeric carrier, and wherein the polymeric carrier comprises a hydrocarbon polymer.

16441. The method of item 16243, wherein the composition comprises a polymeric carrier, and wherein the polymeric carrier comprises a styrene-derived polymer.

16442. The method of item 16243, wherein the composition comprises a polymeric carrier, and wherein the polymeric carrier comprises a butadiene polymer.

16443. The method of item 16243, wherein the composition comprises a polymeric carrier, and wherein the polymeric carrier comprises a macromer.

16444. The method of item 16243, wherein the composition comprises a polymeric carrier, and wherein the polymeric carrier comprises a poly(ethylene glycol)polymer.

16445. The method of item 16243 wherein the composition comprises a polymeric carrier, and wherein the polymeric carrier comprises an amorphous polymer.

16446. The method of item 16243, wherein the device comprises a lubricious coating.

16447. The method of item 16243 wherein the anti-scarring agent is located within pores or holes of the device.

16448. The method of item 16243 wherein the anti-scarring agent is located within a channel, lumen, or divet of the device.

16449. The method of item 16243, wherein the device comprises a second pharmaceutically active agent.

16450. The method of item 16243 wherein the device comprises an anti-inflammatory agent.

16451. The method of item 16243 wherein the device comprises an agent that inhibits infection.

16452. The method of item 16243 wherein the device comprises an agent that inhibits infection, and wherein the agent is an anthracycline.

16453. The method of item 16243 wherein the device comprises an agent that inhibits infection, and wherein the agent is doxorubicin.

16454. The method of item 16243 wherein the device comprises an agent that inhibits infection, and wherein the agent is mitoxantrone.

16455. The method of item 16243 wherein the device comprises an agent that inhibits infection, and wherein the agent is a fluoropyrimidine.

16456. The method of item 16243 wherein the device comprises an agent that inhibits infection, and wherein the agent is 5-fluorouracil (5-FU).

16457. The method of item 16243 wherein the device comprises an agent that inhibits infection, and wherein the agent is a folic acid antagonist.

16458. The method of item 16243 wherein the device comprises an agent that inhibits infection, and wherein the agent is methotrexate.

16459. The method of item 16243 wherein the device comprises an agent that inhibits infection, and wherein the agent is a podophylotoxin.

16460. The method of item 16243 wherein the device comprises an agent that inhibits infection, and wherein the agent is etoposide.

16461. The method of item 16243 wherein the device comprises an agent that inhibits infection, and wherein the agent is a camptothecin.

16462. The method of item 16243 wherein the device comprises an agent that inhibits infection, and wherein the agent is a hydroxyurea.

16463. The method of item 16243 wherein the device comprises an agent that inhibits infection, and wherein the agent is a platinum complex.

16464. The method of item 16243 wherein the device comprises an agent that inhibits infection, and wherein the agent is cisplatin.

16465. The method of item 16243, further comprising an anti-thrombotic agent.

16466. The method of item 16243 wherein the device comprises a visualization agent.

16467. The method of item 16243 wherein the device comprises a visualization agent, wherein the visualization agent is a radiopaque material, and wherein the radiopaque material comprises a metal, a halogenated compound, or a barium containing compound.

16468. The method of item 16243 wherein the device comprises a visualization agent, wherein the visualization agent is a radiopaque material, and wherein the radiopaque material comprises barium, tantalum, or technetium.

16469. The method of item 16243 wherein the device comprises a visualization agent, and wherein the visualization agent is a MRI responsive material.

16470. The method of item 16243 wherein the device comprises a visualization agent, and wherein the visualization agent comprises a gadolinium chelate.

16471. The method of item 16243 wherein the device comprises a visualization agent, and wherein the visualization agent comprises iron, magnesium, manganese, copper, or chromium.

16472. The method of item 16243 wherein the device comprises a visualization agent, and wherein the visualization agent comprises an iron oxide compound.

16473. The method of item 16243 wherein the device comprises a visualization agent, and wherein the visualization agent comprises a dye, pigment, or colorant.

16474. The method of item 16243 wherein the device comprises an echogenic material.

16475. The method of item 16243 wherein the device comprises an echogenic material, and wherein the echogenic material is in the form of a coating.

16476. The method of item 16243 wherein the device is sterile.

16477. The method of item 16243 wherein the anti-scarring agent is released into tissue in the vicinity of the device after deployment of the device.

16478. The method of item 16243 wherein the anti-scarring agent is released into tissue in the vicinity of the device after deployment of the device, and wherein the tissue is connective tissue.

16479. The method of item 16243 wherein the anti-scarring agent is released into tissue in the vicinity of the device after deployment of the device, and wherein the tissue is muscle tissue.

16480. The method of item 16243 wherein the anti-scarring agent is released into tissue in the vicinity of the device after deployment of the device, and wherein the tissue is nerve tissue.

16481. The method of item 16243 wherein the anti-scarring agent is released into tissue in the vicinity of the device after deployment of the device, and wherein the tissue is epithelium tissue.

16482. The method of item 16243 wherein the anti-scarring agent is released in effective concentrations from the device over a period ranging from the time of deployment of the device to about 1 year.

16483. The method of item 16243 wherein the anti-scarring agent is released in effective concentrations from the device over a period ranging from about 1 month to 6 months.

16484. The method of item 16243 wherein the anti-scarring agent is released in effective concentrations from the device over a period ranging from about 1-90 days.

16485. The method of item 16243 wherein the anti-scarring agent is released in effective concentrations from the device at a constant rate.

16486. The method of item 16243 wherein the anti-scarring agent is released in effective concentrations from the device at an increasing rate.

16487. The method of item 16243 wherein the anti-scarring agent is released in effective concentrations from the device at a decreasing rate.

16488. The method of item 16243 wherein the anti-scarring agent is released in effective concentrations from the composition comprising the anti-scarring agent by diffusion over a period ranging from the time of deployment of the device to about 90 days.

16489. The method of item 16243 wherein the anti-scarring agent is released in effective concentrations from the composition comprising the anti-scarring agent by erosion of the composition over a period ranging from the time of deployment of the device to about 90 days.

16490. The method of item 16243 wherein the device comprises about 0.01 μg to about 10 μg of the anti-scarring agent.

16491. The method of item 16243 wherein the device comprises about 10 μg to about 10 mg of the anti-scarring agent.

16492. The method of item 16243 wherein the device comprises about 10 mg to about 250 mg of the anti-scarring agent.

16493. The method of item 16243 wherein the device comprises about 250 mg to about 1000 mg of the anti-scarring agent.

16494. The method of item 16243 wherein the device comprises about 1000 mg to about 2500 mg of the anti-scarring agent.

16495. The method of item 16243 wherein a surface of the device comprises less than 0.01 μg of the anti-scarring agent per mm² of device surface to which the anti-scarring agent is applied.

16496. The method of item 16243 wherein a surface of the device comprises about 0.01 μg to about 1 μg of the anti-scarring agent per mm² of device surface to which the anti-scarring agent is applied.

16497. The method of item 16243 wherein a surface of the device comprises about 1 μg to about 10 μg of the anti-scarring agent per mm² of device surface to which the anti-scarring agent is applied.

16498. The method of item 16243 wherein a surface of the device comprises about 10 μg to about 250 μg of the anti-scarring agent per mm² of device surface to which the anti-scarring agent is applied.

16499. The method of item 16243 wherein a surface of the device comprises about 250 μg to about 1000 μg of the anti-scarring agent of anti-scarring agent per mm² of device surface to which the anti-scarring agent is applied.

16500. The method of item 16243 wherein a surface of the device comprises about 1000 μg to about 2500 μg of the anti-scarring agent per mm² of device surface to which the anti-scarring agent is applied.

16501. The method of item 16243 wherein the combining is performed by direct affixing the agent or the composition to the implant.

16502. The method of item 16243 wherein the combining is performed by spraying the agent or the component onto the implant.

16503. The method of item 16243 wherein the combining is performed by electrospraying the agent or the composition onto the implant.

16504. The method of item 16243 wherein the combining is performed by dipping the implant into a solution comprising the agent or the composition.

16505. The method of item 16243 wherein the combining is performed by covalently attaching the agent or the composition to the implant.

16506. The method of item 16243 wherein the combining is performed by non-covalently attaching the agent or the composition to the implant.

16507. The method of item 16243 wherein the combining is performed by coating the implant with a substance that contains the agent or the composition.

16508. The method of item 16243 wherein the combining is performed by coating the implant with a substance that absorbs the agent.

16509. The method of item 16243 wherein the combining is performed by interweaving a thread composed of, or coated with, the agent or the composition.

16510. The method of item 16243 wherein the combining is performed by covering all the implant with a sleeve that contains the agent or the composition.

16511. The method of item 16243 wherein the combining is performed by covering a portion of the implant with a sleeve that contains the agent or the composition.

16512. The method of item 16243 wherein the combining is performed by covering all the implant with a cover that contains the agent or the composition.

16513. The method of item 16243 wherein the combining is performed by covering a portion of the implant with a cover that contains the agent or the composition.

16514. The method of item 16243 wherein the combining is performed by covering all the implant with an electrospun fabric that contains the agent or the composition.

16515. The method of item 16243 wherein the combining is performed by covering a portion of the implant with an electrospun fabric that contains the agent or the composition.

16516. The method of item 16243 wherein the combining is performed by covering all the implant with a mesh that contains the agent or the composition.

16517. The method of item 16243 wherein the combining is performed by covering a portion of the implant with a mesh that contains the agent or the composition.

16518. The method of item 16243 wherein the combining is performed by constructing all the implant with the agent or the composition.

16519. The method of item 16243 wherein the combining is performed by constructing a portion of the implant with the agent or the composition.

16520. The method of item 16243 wherein the combining is performed by impregnating the implant with the agent or the composition.

16521. The method of item 16243 wherein the combining is performed by constructing all of the implant from a degradable polymer that releases the agent.

16522. The method of item 16243 wherein the combining is performed by constructing a portion of the implant from a degradable polymer that releases the agent.

16523. The method of item 16243 wherein the combining is performed by dipping the implant into a solution that comprise the agent and an inert solvent for the implant.

16524. The method of item 16243 wherein the combining is performed by dipping the implant into a solution that comprises the agent and a solvent that will swill the implant.

16525. The method of item 16243 wherein the combining is performed by dipping the implant into a solution that comprises the agent and a solvent that will dissolve the implant.

16526. The method of item 16243 wherein the combining is performed by dipping the implant into a solution that comprises the agent, a polymer and an inert solvent for the implant.

16527. The method of item 16243 wherein the combining is performed by dipping the implant into a solution that comprises the agent, a polymer and a solvent that will swill the implant.

16528. The method of item 16243 wherein the combining is performed by dipping the implant into a solution that comprises the agent, a polymer and a solvent that will dissolve the implant.

16529. The method of item 16243 wherein the combining is performed by spraying the implant into a solution that comprises the agent and an inert solvent for the implant.

16530. The method of item 16243 wherein the combining is performed by spraying the implant into a solution that comprises the agent and a solvent that will swill the implant.

16531. The method of item 16243 wherein the combining is performed by spraying the implant into a solution that comprises the agent and a solvent that will dissolve the implant.

16532. The method of item 16243 wherein the combining is performed by spraying the implant into a solution that comprises the agent, a polymer and an inert solvent for the implant.

16533. The method of item 16243 wherein the combining is performed by spraying the implant into a solution that comprises the agent, a polymer and a solvent that will swill the implant.

16534. The method of item 16243 wherein the combining is performed by spraying the implant into a solution that comprises the agent, a polymer and a solvent that will dissolve the implant.

16535. A method of making a composition comprising surgical adhesion barrier components and an anti-scarring agent, wherein the composition inhibits formation of surgical adhesions, and wherein the agent inhibits scarring in the vicinity of the composition as it is located within a host that has received the composition.

16536. The method of item 16535 wherein the agent inhibits cell regeneration.

16537. The method of item 16535 wherein the agent inhibits angiogenesis.

16538. The method of item 16535 wherein the agent inhibits fibroblast migration.

16539. The method of item 16535 wherein the agent inhibits fibroblast proliferation.

16540. The method of item 16535 wherein the agent inhibits deposition of extracellular matrix.

16541. The method of item 16535 wherein the agent inhibits tissue remodeling.

16542. The method of item 16535 wherein the agent is an angiogenesis inhibitor.

16543. The method of item 16535 wherein the agent is a 5-lipoxygenase inhibitor or antagonist.

16544. The method of item 16535 wherein the agent is a chemokine receptor antagonist.

16545. The method of item 16535 wherein the agent is a cell cycle inhibitor.

16546. The method of item 16535 wherein the agent is a taxane.

16547. The method of item 16535 wherein the agent is an anti-microtubule agent.

16548. The method of item 16535 wherein the agent is paclitaxel.

16549. The method of item 16535 wherein the agent is not paclitaxel.

16550. The method of item 16535 wherein the agent is an analogue or derivative of paclitaxel.

16551. The method of item 16535 wherein the agent is a vinca alkaloid.

16552. The method of item 16535 wherein the agent is camptothecin or an analogue or derivative thereof.

16553. The method of item 16535 wherein the agent is a podophyllotoxin.

16554. The method of item 16535 wherein the agent is a podophyllotoxin, wherein the podophyllotoxin is etoposide or an analogue or derivative thereof.

16555. The method of item 16535 wherein the agent is an anthracycline.

16556. The method of item 16535 wherein the agent is an anthracycline, wherein the anthracycline is doxorubicin or an analogue or derivative thereof.

16557. The method of item 16535 wherein the agent is an anthracycline, wherein the anthracycline is mitoxantrone or an analogue or derivative thereof.

16558. The method of item 16535 wherein the agent is a platinum compound.

16559. The method of item 16535 wherein the agent is a nitrosourea.

16560. The method of item 16535 wherein the agent is a nitroimidazole.

16561. The method of item 16535 wherein the agent is a folic acid antagonist.

16562. The method of item 16535 wherein the agent is a cytidine analogue.

16563. The method of item 16535 wherein the agent is a pyrimidine analogue.

16564. The method of item 16535 wherein the agent is a fluoropyrimidine analogue.

16565. The method of item 16535 wherein the agent is a purine analogue.

16566. The method of item 16535 wherein the agent is a nitrogen mustard or an analogue or derivative thereof.

16567. The method of item 16535 wherein the agent is a hydroxyurea.

16568. The method of item 16535 wherein the agent is a mytomicin or an analogue or derivative thereof.

16569. The method of item 16535 wherein the agent is an alkyl sulfonate.

16570. The method of item 16535 wherein the agent is a benzamide or an analogue or derivative thereof.

16571. The method of item 16535 wherein the agent is a nicotinamide or an analogue or derivative thereof.

16572. The method of item 16535 wherein the agent is a halogenated sugar or an analogue or derivative thereof.

16573. The method of item 16535 wherein the agent is a DNA alkylating agent.

16574. The method of item 16535 wherein the agent is an anti-microtubule agent.

16575. The method of item 16535 wherein the agent is a topoisomerase inhibitor.

16576. The method of item 16535 wherein the agent is a DNA cleaving agent.

16577. The method of item 16535 wherein the agent is an antimetabolite.

16578. The method of item 16535 wherein the agent inhibits adenosine deaminase.

16579. The method of item 16535 wherein the agent inhibits purine ring synthesis.

16580. The method of item 16535 wherein the agent is a nucleotide interconversion inhibitor.

16581. The method of item 16535 wherein the agent inhibits dihydrofolate reduction.

16582. The method of item 16535 wherein the agent blocks thymidine monophosphate.

16583. The method of item 16535 wherein the agent causes DNA damage.

16584. The method of item 0.16535 wherein the agent is a DNA intercalation agent.

16585. The method of item 16535 wherein the agent is a RNA synthesis inhibitor.

16586. The method of item 16535 wherein the agent is a pyrimidine synthesis inhibitor.

16587. The method of item 16535 wherein the agent inhibits ribonucleotide synthesis or function.

16588. The method of item 16535 wherein the agent inhibits thymidine monophosphate synthesis or function.

16589. The method of item 16535 wherein the agent inhibits DNA synthesis.

16590. The method of item 16535 wherein the agent causes DNA adduct formation.

16591. The method of item 16535 wherein the agent inhibits protein synthesis.

16592. The method of item 16535 wherein the agent inhibits microtubule function.

16593. The method of item 16535 wherein the agent is a cyclin dependent protein kinase inhibitor.

16594. The method of item 16535 wherein the agent is an epidermal growth factor kinase inhibitor.

16595. The method of item 16535 wherein the agent is an elastase inhibitor.

16596. The method of item 16535 wherein the agent is a factor Xa inhibitor.

16597. The method of item 16535 wherein the agent is a farnesyltransferase inhibitor.

16598. The method of item 16535 wherein the agent is a fibrinogen antagonist.

16599. The method of item 16535 wherein the agent is a guanylate cyclase stimulant.

16600. The method of item 16535 wherein the agent is a heat shock protein 90 antagonist.

16601. The method of item 16535 wherein the agent is a heat shock protein 90 antagonist, wherein the heat shock protein 90 antagonist is geldanamycin or an analogue or derivative thereof.

16602. The method of item 16535 wherein the agent is a guanylate cyclase stimulant.

16603. The method of item 16535 wherein the agent is a HMGCoA reductase inhibitor.

16604. The method of item 16535 wherein the agent is a HMGCoA reductase inhibitor, wherein the HMGCoA reductase inhibitor is simvastatin or an analogue or derivative thereof.

16605. The method of item 16535 wherein the agent is a hydroorotate dehydrogenase inhibitor.

16606. The method of item 16535 wherein the agent is an IKK2 inhibitor.

16607. The method of item 16535 wherein the agent is an IL-1 antagonist.

16608. The method of item 16535 wherein the agent is an ICE antagonist.

16609. The method of item 16535 wherein the agent is an IRAK antagonist.

16610. The method of item 16535 wherein the agent is an IL-4 agonist.

16611. The method of item 16535 wherein the agent is an immunomodulatory agent.

16612. The method of item 16535 wherein the agent is sirolimus or an analogue or derivative thereof.

16613. The method of item 16535 wherein the agent is not sirolimus.

16614. The method of item 16535 wherein the agent is everolimus or an analogue or derivative thereof.

16615. The method of item 16535 wherein the agent is tacrolimus or an analogue or derivative thereof.

16616. The method of item 16535 wherein the agent is not tacrolimus.

16617. The method of item 16535 wherein the agent is biolmus or an analogue or derivative thereof.

16618. The method of item 16535 wherein the agent is tresperimus or an analogue or derivative thereof.

16619. The method of item 16535 wherein the agent is auranofin or an analogue or derivative thereof.

16620. The method of item 16535 wherein the agent is 27-0-demethylrapamycin or an analogue or derivative thereof.

16621. The method of item 16535 wherein the agent is gusperimus or an analogue or derivative thereof.

16622. The method of item 16535 wherein the agent is pimecrolimus or an analogue or derivative thereof.

16623. The method of item 16535 wherein the agent is ABT-578 or an analogue or derivative thereof.

16624. The method of item 16535 wherein the agent is an inosine monophosphate dehydrogenase (IMPDH) inhibitor.

16625. The method of item 16535 wherein the agent is an IMPDH inhibitor, wherein the IMPDH inhibitor is mycophenolic acid or an analogue or derivative thereof.

16626. The method of item 16535 wherein the agent is an IMPDH inhibitor, wherein the IMPDH inhibitor is 1-alpha-25 dihydroxy vitamin D₃ or an analogue or derivative thereof.

16627. The method of item 16535 wherein the agent is a leukotriene inhibitor.

16628. The method of item 16535 wherein the agent is a MCP-1 antagonist.

16629. The method of item 16535 wherein the agent is a MMP inhibitor.

16630. The method of item 16535 wherein the agent is an NF kappa B inhibitor.

16631. The method of item 16535 wherein the agent is an NF kappa B inhibitor, wherein the NF kappa B inhibitor is Bay 11-7082.

16632. The method of item 16535 wherein the agent is an NO agonist.

16633. The method of item 16535 wherein the agent is a p38 MAP kinase inhibitor.

16634. The method of item 16535 wherein the agent is a p38 MAP kinase inhibitor, wherein the p38 MAP kinase inhibitor is SB 202190.

16635. The method of item 16535 wherein the agent is a phosphodiesterase inhibitor.

16636. The method of item 16535 wherein the agent is a TGF beta inhibitor.

16637. The method of item 16535 wherein the agent is a thromboxane A2 antagonist.

16638. The method of item 16535 wherein the agent is a TNFa antagonist.

16639. The method of item 16535 wherein the agent is a TACE inhibitor.

16640. The method of item 16535 wherein the agent is a tyrosine kinase inhibitor.

16641. The method of item 16535 wherein the agent is a vitronectin inhibitor.

16642. The method of item 16535 wherein the agent is a fibroblast growth factor inhibitor.

16643. The method of item 16535 wherein the agent is a protein kinase inhibitor.

16644. The method of item 16535 wherein the agent is a PDGF receptor kinase inhibitor.

16645. The method of item 16535 wherein the agent is an endothelial growth factor receptor kinase inhibitor.

16646. The method of item 16535 wherein the agent is a retinoic acid receptor antagonist.

16647. The method of item 16535 wherein the agent is a platelet derived growth factor receptor kinase inhibitor.

16648. The method of item 16535 wherein the agent is a fibronogin antagonist.

16649. The method of item 16535 wherein the agent is an antimycotic agent.

16650. The method of item 16535 wherein the agent is an antimycotic agent, wherein the antimycotic agent is sulconizole.

16651. The method of item 16535 wherein the agent is a bisphosphonate.

16652. The method of item 16535 wherein the agent is a phospholipase A1 inhibitor.

16653. The method of item 16535 wherein the agent is a histamine H1/H2/H3 receptor antagonist.

16654. The method of item 16535 wherein the agent is a macrolide antibiotic.

16655. The method of item 16535 wherein the agent is a GPIIb/IIIa receptor antagonist.

16656. The method of item 16535 wherein the agent is an endothelin receptor antagonist.

16657. The method of item 16535 wherein the agent is a peroxisome proliferator-activated receptor agonist.

16658. The method of item 16535 wherein the agent is an estrogen receptor agent.

16659. The method of item 16535 wherein the agent is a somastostatin analogue.

16660. The method of item 16535 wherein the agent is a neurokinin 1 antagonist.

16661. The method of item 16535 wherein the agent is a neurokinin 3 antagonist.

16662. The method of item 16535 wherein the agent is a VLA-4 antagonist.

16663. The method of item 16535 wherein the agent is an osteoclast inhibitor.

16664. The method of item 16535 wherein the agent is a DNA topoisomerase ATP hydrolyzing inhibitor.

16665. The method of item 16535 wherein the agent is an angiotensin I converting enzyme inhibitor.

16666. The method of item 16535 wherein the agent is an angiotensin II antagonist.

16667. The method of item 16535 wherein the agent is an enkephalinase inhibitor.

16668. The method of item 16535 wherein the agent is a peroxisome proliferator-activated receptor gamma agonist insulin sensitizer.

16669. The method of item 16535 wherein the agent is a protein kinase C inhibitor.

16670. The method of item 16535 wherein the agent is a ROCK (rho-associated kinase) inhibitor.

16671. The method of item 16535 wherein the agent is a CXCR3 inhibitor.

16672. The method of item 16535 wherein the agent is an Itk inhibitor.

16673. The method of item 16535 wherein the agent is a cytosolic phospholipase A₂-alpha inhibitor.

16674. The method of item 16535 wherein the agent is a PPAR agonist.

16675. The method of item 16535 wherein the agent is an immunosuppressant.

16676. The method of item 16535 wherein the agent is an Erb inhibitor.

16677. The method of item 16535 wherein the agent is an apoptosis agonist.

16678. The method of item 16535 wherein the agent is a lipocortin agonist.

16679. The method of item 16535 wherein the agent is a VCAM-1 antagonist.

16680. The method of item 16535 wherein the agent is a collagen antagonist.

16681. The method of item 16535 wherein the agent is an alpha 2 integrin antagonist.

16682. The method of item 16535 wherein the agent is a TNF alpha inhibitor.

16683. The method of item 16535 wherein the agent is a nitric oxide inhibitor.

16684. The method of item 16535 wherein the agent is a cathepsin inhibitor.

16685. The method of item 16535 wherein the agent is not an anti-inflammatory agent.

16686. The method of item 16535 wherein the agent is not a steroid.

16687. The method of item 16535 wherein the agent is not a glucocorticosteroid.

16688. The method of item 16535 wherein the agent is not dexamethasone.

16689. The method of item 16535 wherein the agent is not an anti-infective agent.

16690. The method of item 16535 wherein the agent is not an antibiotic.

16691. The method of item 16535 wherein the agent is not an anti-fungal agent.

16692. The method of item 16535 wherein the components comprise hyaluronic acid or an analog or derivative thereof.

16693. The method of item 16535 wherein the components form a biodegradable polymeric matrix when the composition is administered to the host.

16694. The method of item 16535 wherein the composition is in a sprayable form.

16695. The method of item 16535 wherein the composition is in a gel form.

16696. The method of item 16535 wherein the components have reacted to form a film.

16697. The method of item 16535 wherein the composition is in the form of a film.

16698. The method of item 16535 wherein the components have reacted to form a wrap.

16699. The method of item 16535 wherein the composition is in the form of a wrap.

16700. The method of item 16535 wherein the components have reacted to form a mesh.

16701. The method of item 16535 wherein the composition is in the form of a mesh.

16702. The method of item 16535 wherein the components comprise hyaluronic acid or an analog or derivative thereof.

16703. A device, comprising a central venous catheter implant and an anti-scarring agent or a composition comprising an anti-scarring agent, wherein the agent inhibits scarring between the device and a host into which the device is implanted.

16704. The device of item 16703 wherein the agent inhibits cell regeneration.

16705. The device of item 16703 wherein the agent inhibits angiogenesis.

16706. The device of item 16703 wherein the agent inhibits fibroblast migration.

16707. The device of item 16703 wherein the agent inhibits fibroblast proliferation.

16708. The device of item 16703 wherein the agent inhibits deposition of extracellular matrix.

16709. The device of item 16703 wherein the agent inhibits tissue remodeling.

16710. The device of item 16703 wherein the agent is an angiogenesis inhibitor.

16711. The device of item 16703 wherein the agent is a 5-lipoxygenase inhibitor or antagonist.

16712. The device of item 16703 wherein the agent is a chemokine receptor antagonist.

16713. The device of item 16703 wherein the agent is a cell cycle inhibitor.

16714. The device of item 16703 wherein the agent is a taxane.

16715. The device of item 16703 wherein the agent is an anti-microtubule agent.

16716. The device of item 16703 wherein the agent is paclitaxel.

16717. The device of item 16703 wherein the agent is not paclitaxel.

16718. The device of item 16703 wherein the agent is an analogue or derivative of paclitaxel.

16719. The device of item 16703 wherein the agent is a vinca alkaloid.

16720. The device of item 16703 wherein the agent is camptothecin or an analogue or derivative thereof.

16721. The device of item 16703 wherein the agent is a podophyllotoxin.

16722. The device of item 16703 wherein the agent is a podophyllotoxin, wherein the podophyllotoxin is etoposide or an analogue or derivative thereof.

16723. The device of item 16703 wherein the agent is an anthracycline.

16724. The device of item 16703 wherein the agent is an anthracycline, wherein the anthracycline is doxorubicin or an analogue or derivative thereof.

16725. The device of item 16703 wherein the agent is an anthracycline, wherein the anthracycline is mitoxantrone or an analogue or derivative thereof.

16726. The device of item 16703 wherein the agent is a platinum compound.

16727. The device of item 16703 wherein the agent is a nitrosourea.

16728. The device of item 16703 wherein the agent is a nitroimidazole.

16729. The device of item 16703 wherein the agent is a folic acid antagonist.

16730. The device of item 16703 wherein the agent is a cytidine analogue.

16731. The device of item 16703 wherein the agent is a pyrimidine analogue.

16732. The device of item 16703 wherein the agent is a fluoropyrimidine analogue.

16733. The device of item 16703 wherein the agent is a purine analogue.

16734. The device of item 16703 wherein the agent is a nitrogen mustard or an analogue or derivative thereof.

16735. The device of item 16703 wherein the agent is a hydroxyurea.

16736. The device of item 16703 wherein the agent is a mytomicin or an analogue or derivative thereof.

16737. The device of item 16703 wherein the agent is an alkyl sulfonate.

16738. The device of item 16703 wherein the agent is a benzamide or an analogue or derivative thereof.

16739. The device of item 16703 wherein the agent is a nicotinamide or an analogue or derivative thereof.

16740. The device of item 16703 wherein the agent is a halogenated sugar or an analogue or derivative thereof.

16741. The device of item 16703 wherein the agent is a DNA alkylating agent.

16742. The device of item 16703 wherein the agent is an anti-microtubule agent.

16743. The device of item 16703 wherein the agent is a topoisomerase inhibitor.

16744. The device of item 16703 wherein the agent is a DNA cleaving agent.

16745. The device of item 16703 wherein the agent is an antimetabolite.

16746. The device of item 16703 wherein the agent inhibits adenosine deaminase.

16747. The device of item 16703 wherein the agent inhibits purine ring synthesis.

16748. The device of item 16703 wherein the agent is a nucleotide interconversion inhibitor.

16749. The device of item 16703 wherein the agent inhibits dihydrofolate reduction.

16750. The device of item 16703 wherein the agent blocks thymidine monophosphate.

16751. The device of item 16703 wherein the agent causes DNA damage.

16752. The device of item 16703 wherein the agent is a DNA intercalation agent.

16753. The device of item 16703 wherein the agent is a RNA synthesis inhibitor.

16754. The device of item 16703 wherein the agent is a pyrimidine synthesis inhibitor.

16755. The device of item 16703 wherein the agent inhibits ribonucleotide synthesis or function.

16756. The device of item 16703 wherein the agent inhibits thymidine monophosphate synthesis or function.

16757. The device of item 16703 wherein the agent inhibits DNA synthesis.

16758. The device of item 16703 wherein the agent causes DNA adduct formation.

16759. The device of item 16703 wherein the agent inhibits protein synthesis.

16760. The device of item 16703 wherein the agent inhibits microtubule function.

16761. The device of item 16703 wherein the agent is a cyclin dependent protein kinase inhibitor.

16762. The device of item 16703 wherein the agent is an epidermal growth factor kinase inhibitor.

16763. The device of item 16703 wherein the agent is an elastase inhibitor.

16764. The device of item 16703 wherein the agent is a factor Xa inhibitor.

16765. The device of item 16703 wherein the agent is a farnesyltransferase inhibitor.

16766. The device of item 16703 wherein the agent is a fibrinogen antagonist.

16767. The device of item 16703 wherein the agent is a guanylate cyclase stimulant.

16768. The device of item 16703 wherein the agent is a heat shock protein 90 antagonist.

16769. The device of item 16703 wherein the agent is a heat shock protein 90 antagonist, wherein the heat shock protein 90 antagonist is geldanamycin or an analogue or derivative thereof.

16770. The device of item 16703 wherein the agent is a guanylate cyclase stimulant.

16771. The device of item 16703 wherein the agent is a HMGCoA reductase inhibitor.

16772. The device of item 16703 wherein the agent is a HMGCoA reductase inhibitor, wherein the HMGCoA reductase inhibitor is simvastatin or an analogue or derivative thereof.

16773. The device of item 16703 wherein the agent is a hydroorotate dehydrogenase inhibitor.

16774. The device of item 16703 wherein the agent is an IKK2 inhibitor.

16775. The device of item 16703 wherein the agent is an IL-1 antagonist.

16776. The device of item 16703 wherein the agent is an ICE antagonist.

16777. The device of item 16703 wherein the agent is an IRAK antagonist.

16778. The device of item 16703 wherein the agent is an IL-4 agonist.

16779. The device of item 16703 wherein the agent is an immunomodulatory agent.

16780. The device of item 16703 wherein the agent is sirolimus or an analogue or derivative thereof.

16781. The device of item 16703 wherein the agent is not sirolimus.

16782. The device of item 16703 wherein the agent is everolimus or an analogue or derivative thereof.

16783. The device of item 16703 wherein the agent is tacrolimus or an analogue or derivative thereof.

16784. The device of item 16703 wherein the agent is not tacrolimus.

16785. The device of item 16703 wherein the agent is biolmus or an analogue or derivative thereof.

16786. The device of item 16703 wherein the agent is tresperimus or an analogue or derivative thereof.

16787. The device of item 16703 wherein the agent is auranofin or an analogue or derivative thereof.

16788. The device of item 16703 wherein the agent is 27-0-demethylrapamycin or an analogue or derivative thereof.

16789. The device of item 16703 wherein the agent is gusperimus or an analogue or derivative thereof.

16790. The device of item 16703 wherein the agent is pimecrolimus or an analogue or derivative thereof.

16791. The device of item 16703 wherein the agent is ABT-578 or an analogue or derivative thereof.

16792. The device of item 16703 wherein the agent is an inosine monophosphate dehydrogenase (IMPDH) inhibitor.

16793. The device of item 16703 wherein the agent is an IMPDH inhibitor, wherein the IMPDH inhibitor is mycophenolic acid or an analogue or derivative thereof.

16794. The device of item 16703 wherein the agent is an IMPDH inhibitor, wherein the IMPDH inhibitor is 1-alpha-25 dihydroxy vitamin D₃ or an analogue or derivative thereof.

16795. The device of item 16703 wherein the agent is a leukotriene inhibitor.

16796. The device of item 16703 wherein the agent is a MCP-1 antagonist.

16797. The device of item 16703 wherein the agent is a MMP inhibitor.

16798. The device of item 16703 wherein the agent is an NF kappa B inhibitor.

16799. The device of item 16703 wherein the agent is an NF kappa B inhibitor, wherein the NF kappa B inhibitor is Bay 11-7082.

16800. The device of item 16703 wherein the agent is an NO agonist.

16801. The device of item 16703 wherein the agent is a p38 MAP kinase inhibitor.

16802. The device of item 16703 wherein the agent is a p38 MAP kinase inhibitor, wherein the p38 MAP kinase inhibitor is SB 202190.

16803. The device of item 16703 wherein the agent is a phosphodiesterase inhibitor.

16804. The device of item 16703 wherein the agent is a TGF beta inhibitor.

16805. The device of item 16703 wherein the agent is a thromboxane A2 antagonist.

16806. The device of item 16703 wherein the agent is a TNFa antagonist.

16807. The device of item 16703 wherein the agent is a TACE inhibitor.

16808. The device of item 16703 wherein the agent is a tyrosine kinase inhibitor.

16809. The device of item 16703 wherein the agent is a vitronectin inhibitor.

16810. The device of item 16703 wherein the agent is a fibroblast growth factor inhibitor.

16811. The device of item 16703 wherein the agent is a protein kinase inhibitor.

16812. The device of item 16703 wherein the agent is a PDGF receptor kinase inhibitor.

16813. The device of item 16703 wherein the agent is an endothelial growth factor receptor kinase inhibitor.

16814. The device of item 16703 wherein the agent is a retinoic acid receptor antagonist.

16815. The device of item 16703 wherein the agent is a platelet derived growth factor receptor kinase inhibitor.

16816. The device of item 16703 wherein the agent is a fibronogin antagonist.

16817. The device of item 16703 wherein the agent is an antimycotic agent.

16818. The device of item 16703 wherein the agent is an antimycotic agent, wherein the antimycotic agent is sulconizole.

16819. The device of item 16703 wherein the agent is a bisphosphonate.

16820. The device of item 16703 wherein the agent is a phospholipase A1 inhibitor.

16821. The device of item 16703 wherein the agent is a histamine H1/H2/H3 receptor antagonist.

16822. The device of item 16703 wherein the agent is a macrolide antibiotic.

16823. The device of item 16703 wherein the agent is a GPIIb/IIIa receptor antagonist.

16824. The device of item 16703 wherein the agent is an endothelin receptor antagonist.

16825. The device of item 16703 wherein the agent is a peroxisome proliferator-activated receptor agonist.

16826. The device of item 16703 wherein the agent is an estrogen receptor agent.

16827. The device of item 16703 wherein the agent is a somastostatin analogue.

16828. The device of item 16703 wherein the agent is a neurokinin 1 antagonist.

16829. The device of item 16703 wherein the agent is a neurokinin 3 antagonist.

16830. The device of item 16703 wherein the agent is a VLA-4 antagonist.

16831. The device of item 16703 wherein the agent is an osteoclast inhibitor.

16832. The device of item 16703 wherein the agent is a DNA topoisomerase ATP hydrolyzing inhibitor.

16833. The device of item 16703 wherein the agent is an angiotensin I converting enzyme inhibitor.

16834. The device of item 16703 wherein the agent is an angiotensin II antagonist.

16835. The device of item 16703 wherein the agent is an enkephalinase inhibitor.

16836. The device of item 16703 wherein the agent is a peroxisome proliferator-activated receptor gamma agonist insulin sensitizer.

16837. The device of item 16703 wherein the agent is a protein kinase C inhibitor.

16838. The device of item 16703 wherein the agent is a ROCK (rho-associated kinase) inhibitor.

16839. The device of item 16703 wherein the agent is a CXCR3 inhibitor.

16840. The device of item 16703 wherein the agent is an Itk inhibitor.

16841. The device of item 16703 wherein the agent is a cytosolic phospholipase A₂-alpha inhibitor.

16842. The device of item 16703 wherein the agent is a PPAR agonist.

16843. The device of item 16703 wherein the agent is an immunosuppressant.

16844. The device of item 16703 wherein the agent is an Erb inhibitor.

16845. The device of item 16703 wherein the agent is an apoptosis agonist.

16846. The device of item 16703 wherein the agent is a lipocortin agonist.

16847. The device of item 16703 wherein the agent is a VCAM-1 antagonist.

16848. The device of item 16703 wherein the agent is a collagen antagonist.

16849. The device of item 16703 wherein the agent is an alpha 2 integrin antagonist.

16850. The device of item 16703 wherein the agent is a TNF alpha inhibitor.

16851. The device of item 16703 wherein the agent is a nitric oxide inhibitor.

16852. The device of item 16703 wherein the agent is a cathepsin inhibitor.

16853. The device of item 16703 wherein the agent is not an anti-inflammatory agent.

16854. The device of item 16703 wherein the agent is not a steroid.

16855. The device of item 16703 wherein the agent is not a glucocorticosteroid.

16856. The device of item 16703 wherein the agent is not dexamethasone.

16857. The device of item 16703 wherein the agent is not an anti-infective agent.

16858. The device of item 16703 wherein the agent is not an antibiotic.

16859. The device of item 16703 wherein the agent is not an anti-fungal agent.

16860. The device of item 16703, further comprising a polymer.

16861. The device of item 16703, further comprising a polymeric carrier.

16862. The device of item 16703 wherein the anti-scarring agent inhibits adhesion between the device and a host into which the device is implanted.

16863. The device of item 16703 wherein the device delivers the anti-scarring agent locally to tissue proximate to the device.

16864. The device of item 16703, further comprising a coating, wherein the coating comprises the anti-scarring agent.

16865. The device of item 16703, further comprising a coating, wherein the coating is disposed on a surface of the device.

16866. The device of item 16703, further comprising a coating, wherein the coating directly contacts the device.

16867. The device of item 16703, further comprising a coating, wherein the coating indirectly contacts the device.

16868. The device of item 16703, further comprising a coating, wherein the coating partially covers the device.

16869. The device of item 16703, further comprising a coating, wherein the coating completely covers the device.

16870. The device of item 16703, further comprising a coating, wherein the coating is a uniform coating.

16871. The device of item 16703, further comprising a coating, wherein the coating is a non-uniform coating.

16872. The device of item 16703, further comprising a coating, wherein the coating is a discontinuous coating.

16873. The device of item 16703, further comprising a coating, wherein the coating is a patterned coating.

16874. The device of item 16703, further comprising a coating, wherein the coating has a thickness of 100 μm or less.

16875. The device of item 16703, further comprising a coating, wherein the coating has a thickness of 10 μm or less.

16876. The device of item 16703, further comprising a coating, wherein the coating adheres to the surface of the device upon deployment of the device.

16877. The device of item 16703, further comprising a coating, wherein the coating is stable at room temperature for a period of 1 year.

16878. The device of item 16703, further comprising a coating, wherein the anti-scarring agent is present in the coating in an amount ranging between about 0.0001% to about 1% by weight.

16879. The device of item 16703, further comprising a coating, wherein the anti-scarring agent is present in the coating in an amount ranging between about 1% to about 10% by weight.

16880. The device of item 16703, further comprising a coating, wherein the anti-scarring agent is present in the coating in an amount ranging between about 10% to about 25% by weight.

16881. The device of item 16703, further comprising a coating, wherein the anti-scarring agent is present in the coating in an amount ranging between about 25% to about 70% by weight.

16882. The device of item 16703, further comprising a coating, wherein the coating further comprises a polymer.

16883. The device of item 16703, further comprising a first coating having a first composition and the second coating having a second composition.

16884. The device of item 16703, further comprising a first coating having a first composition and the second coating having a second composition, wherein the first composition and the second composition are different.

16885. The device of item 16703, further comprising a polymer.

16886. The device of item 16703, further comprising a polymeric carrier.

16887. The device of item 16703, further comprising a polymeric carrier, wherein the polymeric carrier comprises a copolymer.

16888. The device of item 16703, further comprising a polymeric carrier, wherein the polymeric carrier comprises a block copolymer.

16889. The device of item 16703, further comprising a polymeric carrier, wherein the polymeric carrier comprises a random copolymer.

16890. The device of item 16703, further comprising a polymeric carrier, wherein the polymeric carrier comprises a biodegradable polymer.

16891. The device of item 16703, further comprising a polymeric carrier, wherein the polymeric carrier comprises a non-biodegradable polymer.

16892. The device of item 16703, further comprising a polymeric carrier, wherein the polymeric carrier comprises a hydrophilic polymer.

16893. The device of item 16703, further comprising a polymeric carrier, wherein the polymeric carrier comprises a hydrophobic polymer.

16894. The device of item 16703, further comprising a polymeric carrier, wherein the polymeric carrier comprises a polymer having hydrophilic domains.

16895. The device of item 16703, further comprising a polymeric carrier, wherein the polymeric carrier comprises a polymer having hydrophobic domains.

16896. The device of item 16703, further comprising a polymeric carrier, wherein the polymeric carrier comprises a non-conductive polymer.

16897. The device of item 16703, further comprising a polymeric carrier, wherein the polymeric carrier comprises an elastomer.

16898. The device of item 16703, further comprising a polymeric carrier, wherein the polymeric carrier comprises a hydrogel.

16899. The device of item 16703, further comprising a polymeric carrier, wherein the polymeric carrier comprises a silicone polymer.

16900. The device of item 16703, further comprising a polymeric carrier, wherein the polymeric carrier comprises a hydrocarbon polymer.

16901. The device of item 16703, further comprising a polymeric carrier, wherein the polymeric carrier comprises a styrene-derived polymer.

16902. The device of item 16703, further comprising a polymeric carrier, wherein the polymeric carrier comprises a butadiene polymer.

16903. The device of item 16703, further comprising a polymeric carrier, wherein the polymeric carrier comprises a macromer.

16904. The device of item 16703, further comprising a polymeric carrier, wherein the polymeric carrier comprises a poly(ethylene glycol) polymer.

16905. The device of item 16703, further comprising a polymeric carrier, wherein the polymeric carrier comprises an amorphous polymer.

16906. The device of item 16703, further comprising a lubricious coating.

16907. The device of item 16703 wherein the anti-scarring agent is located within pores or holes of the device.

16908. The device of item 16703 wherein the anti-scarring agent is located within a channel, lumen, or divet of the device.

16909. The device of item 16703, further comprising a second pharmaceutically active agent.

16910. The device of item 16703, further comprising an anti-inflammatory agent.

16911. The device of item 16703, further comprising an agent that inhibits infection.

16912. The device of item 16703, further comprising an agent that inhibits infection, wherein the agent is an anthracycline.

16913. The device of item 16703, further comprising an agent that inhibits infection, wherein the agent is doxorubicin.

16914. The device of item 16703, further comprising an agent that inhibits infection, wherein the agent is mitoxantrone.

16915. The device of item 16703, further comprising an agent that inhibits infection, wherein the agent is a fluoropyrimidine.

16916. The device of item 16703, further comprising an agent that inhibits infection, wherein the agent is 5-fluorouracil (5-FU).

16917. The device of item 16703, further comprising an agent that inhibits infection, wherein the agent is a folic acid antagonist.

16918. The device of item 16703, further comprising an agent that inhibits infection, wherein the agent is methotrexate.

16919. The device of item 16703, further comprising an agent that inhibits infection, wherein the agent is a podophylotoxin.

16920. The device of item 16703, further comprising an agent that inhibits infection, wherein the agent is etoposide.

16921. The device of item 16703, further comprising an agent that inhibits infection, wherein the agent is a camptothecin.

16922. The device of item 16703, further comprising an agent that inhibits infection, wherein the agent is a hydroxyurea.

16923. The device of item 16703, further comprising an agent that inhibits infection, wherein the agent is a platinum complex.

16924. The device of item 16703, further comprising an agent that inhibits infection, wherein the agent is cisplatin.

16925. The device of item 16703, further comprising an anti-thrombotic agent.

16926. The device of item 16703, further comprising a visualization agent.

16927. The device of item 16703, further comprising a visualization agent, wherein the visualization agent is a radiopaque material, wherein the radiopaque material comprises a metal, a halogenated compound, or a barium containing compound.

16928. The device of item 16703, further comprising a visualization agent, wherein the visualization agent is a radiopaque material, wherein the radiopaque material comprises barium, tantalum, or technetium.

16929. The device of item 16703, further comprising a visualization agent, wherein the visualization agent is a MRI responsive material.

16930. The device of item 16703, further comprising a visualization agent, wherein the visualization agent comprises a gadolinium chelate.

16931. The device of item 16703, further comprising a visualization agent, wherein the visualization agent comprises iron, magnesium, manganese, copper, or chromium.

16932. The device of item 16703, further comprising a visualization agent, wherein the visualization agent comprises an iron oxide compound.

16933. The device of item 16703, further comprising a visualization agent, wherein the visualization agent comprises a dye, pigment, or colorant.

16934. The device of item 16703, further comprising an echogenic material.

16935. The device of item 16703, further comprising an echogenic material, wherein the echogenic material is in the form of a coating.

16936. The device of item 16703 wherein the device is sterile.

16937. The device of item 16703 wherein the anti-scarring agent is released into tissue in the vicinity of the device after deployment of the device.

16938. The device of item 16703 wherein the anti-scarring agent is released into tissue in the vicinity of the device after deployment of the device, wherein the tissue is connective tissue.

16939. The device of item 16703 wherein the anti-scarring agent is released into tissue in the vicinity of the device after deployment of the device, wherein the tissue is muscle tissue.

16940. The device of item 16703 wherein the anti-scarring agent is released into tissue in the vicinity of the device after deployment of the device, wherein the tissue is nerve tissue.

16941. The device of item 16703 wherein the anti-scarring agent is released into tissue in the vicinity of the device after deployment of the device, wherein the tissue is epithelium tissue.

16942. The device of item 16703 wherein the anti-scarring agent is released in effective concentrations from the device over a period ranging from the time of deployment of the device to about 1 year.

16943. The device of item 16703 wherein the anti-scarring agent is released in effective concentrations from the device over a period ranging from about 1 month to 6 months.

16944. The device of item 16703 wherein the anti-scarring agent is released in effective concentrations from the device over a period ranging from about 1-90 days.

16945. The device of item 16703 wherein the anti-scarring agent is released in effective concentrations from the device at a constant rate.

16946. The device of item 16703 wherein the anti-scarring agent is released in effective concentrations from the device at an increasing rate.

16947. The device of item 16703 wherein the anti-scarring agent is released in effective concentrations from the device at a decreasing rate.

16948. The device of item 16703 wherein the anti-scarring agent is released in effective concentrations from the composition comprising the anti-scarring agent by diffusion over a period ranging from the time of deployment of the device to about 90 days.

16949. The device of item 16703 wherein the anti-scarring agent is released in effective concentrations from the composition comprising the anti-scarring agent by erosion of the composition over a period ranging from the time of deployment of the device to about 90 days.

16950. The device of item 16703 wherein the device comprises about 0.01 μg to about 10 μg of the anti-scarring agent.

16951. The device of item 16703 wherein the device comprises about 10 μg to about 10 mg of the anti-scarring agent.

16952. The device of item 16703 wherein the device comprises about 10 mg to about 250 mg of the anti-scarring agent.

16953. The device of item 16703 wherein the device comprises about 250 mg to about 1000 mg of the anti-scarring agent.

16954. The device of item 16703 wherein the device comprises about 1000 mg to about 2500 mg of the anti-scarring agent.

16955. The device of item 16703 wherein a surface of the device comprises less than 0.01 μg of the anti-scarring agent per mm² of device surface to which the anti-scarring agent is applied.

16956. The device of item 16703 wherein a surface of the device comprises about 0.01 μg to about 1 μg of the anti-scarring agent per mm² of device surface to which the anti-scarring agent is applied.

16957. The device of item 16703 wherein a surface of the device comprises about 1 μg to about 10 μg of the anti-scarring agent per mm² of device surface to which the anti-scarring agent is applied.

16958. The device of item 16703 wherein a surface of the device comprises about 10 μg to about 250 μg of the anti-scarring agent per mm² of device surface to which the anti-scarring agent is applied.

16959. The device of item 16703 wherein a surface of the device comprises about 250 μg to about 1000 μg of the anti-scarring agent of anti-scarring agent per mm² of device surface to which the anti-scarring agent is applied.

16960. The device of item 16703 wherein a surface of the device comprises about 1000 μg to about 2500 μg of the anti-scarring agent per mm² of device surface to which the anti-scarring agent is applied.

16961. The device of item 16703 wherein the implant is a total parenteral nutrition catheter.

16962. The device of item 16703 wherein the implant is a flow-directed balloon-tipped pulmonary artery catheter.

16963. A method for inhibiting scarring comprising placing a central venous catheter implant and an anti-scarring agent or a composition comprising an anti-scarring agent into an animal host, wherein the agent inhibits scarring.

16964. The method of item 16963 wherein the agent inhibits cell regeneration.

16965. The method of item 16963 wherein the agent inhibits angiogenesis.

16966. The method of item 16963 wherein the agent inhibits fibroblast migration.

16967. The method of item 16963 wherein the agent inhibits fibroblast proliferation.

16968. The method of item 16963 wherein the agent inhibits deposition of extracellular matrix.

16969. The method of item 16963 wherein the agent inhibits tissue remodeling.

16970. The method of item 16963 wherein the agent is an angiogenesis inhibitor.

16971. The method of item 16963 wherein the agent is a 5-lipoxygenase inhibitor or antagonist.

16972. The method of item 16963 wherein the agent is a chemokine receptor antagonist.

16973. The method of item 16963 wherein the agent is a cell cycle inhibitor.

16974. The method of item 16963 wherein the agent is a taxane.

16975. The method of item 16963 wherein the agent is an anti-microtubule agent.

16976. The method of item 16963 wherein the agent is paclitaxel.

16977. The method of item 16963 wherein the agent is not paclitaxel.

16978. The method of item 16963 wherein the agent is an analogue or derivative of paclitaxel.

16979. The method of item 16963 wherein the agent is a vinca alkaloid.

16980. The method of item 16963 wherein the agent is camptothecin or an analogue or derivative thereof.

16981. The method of item 16963 wherein the agent is a podophyllotoxin.

16982. The method of item 16963 wherein the agent is a podophyllotoxin, wherein the podophyllotoxin is etoposide or an analogue or derivative thereof.

16983. The method of item 16963 wherein the agent is an anthracycline.

16984. The method of item 16963 wherein the agent is an anthracycline, wherein the anthracycline is doxorubicin or an analogue or derivative thereof.

16985. The method of item 16963 wherein the agent is an anthracycline, wherein the anthracycline is mitoxantrone or an analogue or derivative thereof.

16986. The method of item 16963 wherein the agent is a platinum compound.

16987. The method of item 16963 wherein the agent is a nitrosourea.

16988. The method of item 16963 wherein the agent is a nitroimidazole.

16989. The method of item 16963 wherein the agent is a folic acid antagonist.

16990. The method of item 16963 wherein the agent is a cytidine analogue.

16991. The method of item 16963 wherein the agent is a pyrimidine analogue.

16992. The method of item 16963 wherein the agent is a fluoropyrimidine analogue.

16993. The method of item 16963 wherein the agent is a purine analogue.

16994. The method of item 16963 wherein the agent is a nitrogen mustard or an analogue or derivative thereof.

16995. The method of item 16963 wherein the agent is a hydroxyurea.

16996. The method of item 16963 wherein the agent is a mytomicin or an analogue or derivative thereof.

16997. The method of item 16963 wherein the agent is an alkyl sulfonate.

16998. The method of item 16963 wherein the agent is a benzamide or an analogue or derivative thereof.

16999. The method of item 16963 wherein the agent is a nicotinamide or an analogue or derivative thereof.

17000. The method of item 16963 wherein the agent is a halogenated sugar or an analogue or derivative thereof.

17001. The method of item 16963 wherein the agent is a DNA alkylating agent.

17002. The method of item 16963 wherein the agent is an anti-microtubule agent.

17003. The method of item 16963 wherein the agent is a topoisomerase inhibitor.

17004. The method of item 16963 wherein the agent is a DNA cleaving agent.

17005. The method of item 16963 wherein the agent is an antimetabolite.

17006. The method of item 16963 wherein the agent inhibits adenosine deaminase.

17007. The method of item 16963 wherein the agent inhibits purine ring synthesis.

17008. The method of item 16963 wherein the agent is a nucleotide interconversion inhibitor.

17009. The method of item 16963 wherein the agent inhibits dihydrofolate reduction.

17010. The method of item 16963 wherein the agent blocks thymidine monophosphate.

17011. The method of item 16963 wherein the agent causes DNA damage.

17012. The method of item 16963 wherein the agent is a DNA intercalation agent.

17013. The method of item 16963 wherein the agent is a RNA synthesis inhibitor.

17014. The method of item 16963 wherein the agent is a pyrimidine synthesis inhibitor.

17015. The method of item 16963 wherein the agent inhibits ribonucleotide synthesis or function.

17016. The method of item 16963 wherein the agent inhibits thymidine monophosphate synthesis or function.

17017. The method of item 16963 wherein the agent inhibits DNA synthesis.

17018. The method of item 16963 wherein the agent causes DNA adduct formation.

17019. The method of item 16963 wherein the agent inhibits protein synthesis.

17020. The method of item 16963 wherein the agent inhibits microtubule function.

17021. The method of item 16963 wherein the agent is a cyclin dependent protein kinase inhibitor.

17022. The method of item 16963 wherein the agent is an epidermal growth factor kinase inhibitor.

17023. The method of item 16963 wherein the agent is an elastase inhibitor.

17024. The method of item 16963 wherein the agent is a factor Xa inhibitor.

17025. The method of item 16963 wherein the agent is a farnesyltransferase inhibitor.

17026. The method of item 16963 wherein the agent is a fibrinogen antagonist.

17027. The method of item 16963 wherein the agent is a guanylate cyclase stimulant.

17028. The method of item 16963 wherein the agent is a heat shock protein 90 antagonist.

17029. The method of item 16963 wherein the agent is a heat shock protein 90 antagonist, wherein the heat shock protein 90 antagonist is geldanamycin or an analogue or derivative thereof.

17030. The method of item 16963 wherein the agent is a guanylate cyclase stimulant.

17031. The method of item 16963 wherein the agent is a HMGCoA reductase inhibitor.

17032. The method of item 16963 wherein the agent is a HMGCoA reductase inhibitor, wherein the HMGCoA reductase inhibitor is simvastatin or an analogue or derivative thereof.

17033. The method of item 16963 wherein the agent is a hydroorotate dehydrogenase inhibitor.

17034. The method of item 16963 wherein the agent is an IKK2 inhibitor.

17035. The method of item 16963 wherein the agent is an IL-1 antagonist.

17036. The method of item 16963 wherein the agent is an ICE antagonist.

17037. The method of item 16963 wherein the agent is an IRAK antagonist.

17038. The method of item 16963 wherein the agent is an IL-4 agonist.

17039. The method of item 16963 wherein the agent is an immunomodulatory agent.

17040. The method of item 16963 wherein the agent is sirolimus or an analogue or derivative thereof.

17041. The method of item 16963 wherein the agent is not sirolimus.

17042. The method of item 16963 wherein the agent is everolimus or an analogue or derivative thereof.

17043. The method of item 16963 wherein the agent is tacrolimus or an analogue or derivative thereof.

17044. The method of item 16963 wherein the agent is not tacrolimus.

17045. The method of item 16963 wherein the agent is biolmus or an analogue or derivative thereof.

17046. The method of item 16963 wherein the agent is tresperimus or an analogue or derivative thereof.

17047. The method of item 16963 wherein the agent is auranofin or an analogue or derivative thereof.

17048. The method of item 16963 wherein the agent is 27-0-demethylrapamycin or an analogue or derivative thereof.

17049. The method of item 16963 wherein the agent is gusperimus or an analogue or derivative thereof.

17050. The method of item 16963 wherein the agent is pimecrolimus or an analogue or derivative thereof.

17051. The method of item 16963 wherein the agent is ABT-578 or an analogue or derivative thereof.

17052. The method of item 16963 wherein the agent is an inosine monophosphate dehydrogenase (IMPDH) inhibitor.

17053. The method of item 16963 wherein the agent is an IMPDH inhibitor, wherein the IMPDH inhibitor is mycophenolic acid or an analogue or derivative thereof.

17054. The method of item 16963 wherein the agent is an IMPDH inhibitor, wherein the IMPDH inhibitor is 1-alpha-25 dihydroxy vitamin D₃ or an analogue or derivative thereof.

17055. The method of item 16963 wherein the agent is a leukotriene inhibitor.

17056. The method of item 16963 wherein the agent is a MCP-1 antagonist.

17057. The method of item 16963 wherein the agent is a MMP inhibitor.

17058. The method of item 16963 wherein the agent is an NF kappa B inhibitor.

17059. The method of item 16963 wherein the agent is an NF kappa B inhibitor, wherein the NF kappa B inhibitor is Bay 11-7082.

17060. The method of item 16963 wherein the agent is an NO agonist.

17061. The method of item 16963 wherein the agent is a p38 MAP kinase inhibitor.

17062. The method of item 16963 wherein the agent is a p38 MAP kinase inhibitor, wherein the p38 MAP kinase inhibitor is SB 202190.

17063. The method of item 16963 wherein the agent is a phosphodiesterase inhibitor.

17064. The method of item 16963 wherein the agent is a TGF beta inhibitor.

17065. The method of item 16963 wherein the agent is a thromboxane A2 antagonist.

17066. The method of item 16963 wherein the agent is a TNFa antagonist.

17067. The method of item 16963 wherein the agent is a TACE inhibitor.

17068. The method of item 16963 wherein the agent is a tyrosine kinase inhibitor.

17069. The method of item 16963 wherein the agent is a vitronectin inhibitor.

17070. The method of item 16963 wherein the agent is a fibroblast growth factor inhibitor.

17071. The method of item 16963 wherein the agent is a protein kinase inhibitor.

17072. The method of item 16963 wherein the agent is a PDGF receptor kinase inhibitor.

17073. The method of item 16963 wherein the agent is an endothelial growth factor receptor kinase inhibitor.

17074. The method of item 16963 wherein the agent is a retinoic acid receptor antagonist.

17075. The method of item 16963 wherein the agent is a platelet derived growth factor receptor kinase inhibitor.

17076. The method of item 16963 wherein the agent is a fibronogin antagonist.

17077. The method of item 16963 wherein the agent is an antimycotic agent.

17078. The method of item 16963 wherein the agent is an antimycotic agent, wherein the antimycotic agent is sulconizole.

17079. The method of item 16963 wherein the agent is a bisphosphonate.

17080. The method of item 16963 wherein the agent is a phospholipase A1 inhibitor.

17081. The method of item 16963 wherein the agent is a histamine H1/H2/H3 receptor antagonist.

17082. The method of item 16963 wherein the agent is a macrolide antibiotic.

17083. The method of item 16963 wherein the agent is a GPIIb/IIIa receptor antagonist.

17084. The method of item 16963 wherein the agent is an endothelin receptor antagonist.

17085. The method of item 16963 wherein the agent is a peroxisome proliferator-activated receptor agonist.

17086. The method of item 16963 wherein the agent is an estrogen receptor agent.

17087. The method of item 16963 wherein the agent is a somastostatin analogue.

17088. The method of item 16963 wherein the agent is a neurokinin 1 antagonist.

17089. The method of item 16963 wherein the agent is a neurokinin 3 antagonist.

17090. The method of item 16963 wherein the agent is a VLA-4 antagonist.

17091. The method of item 16963 wherein the agent is an osteoclast inhibitor.

17092. The method of item 16963 wherein the agent is a DNA topoisomerase ATP hydrolyzing inhibitor.

17093. The method of item 16963 wherein the agent is an angiotensin I converting enzyme inhibitor.

17094. The method of item 16963 wherein the agent is an angiotensin II antagonist.

17095. The method of item 16963 wherein the agent is an enkephalinase inhibitor.

17096. The method of item 16963 wherein the agent is a peroxisome proliferator-activated receptor gamma agonist insulin sensitizer.

17097. The method of item 16963 wherein the agent is a protein kinase C inhibitor.

17098. The method of item 16963 wherein the agent is a ROCK (rho-associated kinase) inhibitor.

17099. The method of item 16963 wherein the agent is a CXCR3 inhibitor.

17100. The method of item 16963 wherein the agent is an Itk inhibitor.

17101. The method of item 16963 wherein the agent is a cytosolic phospholipase A₂-alpha inhibitor.

17102. The method of item 16963 wherein the agent is a PPAR agonist.

17103. The method of item 16963 wherein the agent is an immunosuppressant.

17104. The method of item 16963 wherein the agent is an Erb inhibitor.

17105. The method of item 16963 wherein the agent is an apoptosis agonist.

17106. The method of item 16963 wherein the agent is a lipocortin agonist.

17107. The method of item 16963 wherein the agent is a VCAM-1 antagonist.

17108. The method of item 16963 wherein the agent is a collagen antagonist.

17109. The method of item 16963 wherein the agent is an alpha 2 integrin antagonist.

17110. The method of item 16963 wherein the agent is a TNF alpha inhibitor.

17111. The method of item 16963 wherein the agent is a nitric oxide inhibitor.

17112. The method of item 16963 wherein the agent is a cathepsin inhibitor.

17113. The method of item 16963 wherein the agent is not an anti-inflammatory agent.

17114. The method of item 16963 wherein the agent is not a steroid.

17115. The method of item 16963 wherein the agent is not a glucocorticosteroid.

17116. The method of item 16963 wherein the agent is not dexamethasone.

17117. The method of item 16963 wherein the agent is not an anti-infective agent.

17118. The method of item 16963 wherein the agent is not an antibiotic.

17119. The method of item 16963 wherein the agent is not an anti-fungal agent.

17120. The method of item 16963, wherein the composition comprises a polymer.

17121. The method of item 16963, wherein the composition comprises a polymer, and the polymer is, or comprises, a copolymer.

17122. The method of item 16963, wherein the composition comprises a polymer, and the polymer is, or comprises, a block copolymer.

17123. The method of item 16963, wherein the composition comprises a polymer, and the polymer is, or comprises, a random copolymer.

17124. The method of item 16963, wherein the composition comprises a polymer, and the polymer is, or comprises, a biodegradable polymer.

17125. The method of item 16963, wherein the composition comprises a polymer, and the polymer is, or comprises, a non-biodegradable polymer.

17126. The method of item 16963, wherein the composition comprises a polymer, and the polymer is, or comprises, a hydrophilic polymer.

17127. The method of item 16963, wherein the composition comprises a polymer, and the polymer is, or comprises, a hydrophobic polymer.

17128. The method of item 16963, wherein the composition comprises a polymer, and the polymer is, or comprises, a polymer having hydrophilic domains.

17129. The method of item 16963, wherein the composition comprises a polymer, and the polymer is, or comprises, a polymer having hydrophobic domains.

17130. The method of item 16963, wherein the composition comprises a polymer, and the polymer is, or comprises, a non-conductive polymer.

17131. The method of item 16963, wherein the composition comprises a polymer, and the polymer is, or comprises, an elastomer.

17132. The method of item 16963, wherein the composition comprises a polymer, and the polymer is, or comprises, a hydrogel.

17133. The method of item 16963, wherein the composition comprises a polymer, and the polymer is, or comprises, a silicone polymer.

17134. The method of item 16963, wherein the composition comprises a polymer, and the polymer is, or comprises, a hydrocarbon polymer.

17135. The method of item 16963, wherein the composition comprises a polymer, and the polymer is, or comprises, a styrene-derived polymer.

17136. The method of item 16963, wherein the composition comprises a polymer, and the polymer is, or comprises, a butadiene-derived polymer.

17137. The method of item 16963, wherein the composition comprises a polymer, and the polymer is, or comprises, a macromer.

17138. The method of item 16963, wherein the composition comprises a polymer, and the polymer is, or comprises, a poly(ethylene glycol) polymer.

17139. The method of item 16963, wherein the composition comprises a polymer, and the polymer is, or comprises, an amorphous polymer.

17140. The method of item 16963, wherein the composition further comprises a second pharmaceutically active agent.

17141. The method of item 16963, wherein the composition further comprises an anti-inflammatory agent.

17142. The method of item 16963, wherein the composition further comprises an agent that inhibits infection.

17143. The method of item 16963, wherein the composition further comprises an anthracycline.

17144. The method of item 16963, wherein the composition further comprises doxorubicin.

17145. The method of item 16963 wherein the composition further comprises mitoxantrone.

17146. The method of item 16963 wherein the composition further comprises a fluoropyrimidine.

17147. The method of item 16963, wherein the composition further comprises 5-fluorouracil (5-FU).

17148. The method of item 16963, wherein the composition further comprises a folic acid antagonist.

17149. The method of item 16963, wherein the composition further comprises methotrexate.

17150. The method of item 16963, wherein the composition further comprises a podophylotoxin.

17151. The method of item 16963, wherein the composition further comprises etoposide.

17152. The method of item 16963, wherein the composition further comprises camptothecin.

17153. The method of item 16963, wherein the composition further comprises a hydroxyurea.

17154. The method of item 16963, wherein the composition further comprises a platinum complex.

17155. The method of item 16963, wherein the composition further comprises cisplatin.

17156. The method of item 16963 wherein the composition further comprises an anti-thrombotic agent.

17157. The method of item 16963, wherein the composition further comprises a visualization agent.

17158. The method of item 16963, wherein the composition further comprises a visualization agent, and the visualization agent is a radiopaque material, wherein the radiopaque material comprises a metal, a halogenated compound, or a barium containing compound.

17159. The method of item 16963, wherein the composition further comprises a visualization agent, and the visualization agent is, or comprises, barium, tantalum, or technetium.

17160. The method of item 16963, wherein the composition further comprises a visualization agent, and the visualization agent is, or comprises, an MRI responsive material.

17161. The method of item 16963, wherein the composition further comprises a visualization agent, and the visualization agent is, or comprises, a gadolinium chelate.

17162. The method of item 16963, wherein the composition further comprises a visualization agent, and the visualization agent is, or comprises, iron, magnesium, manganese, copper, or chromium.

17163. The method of item 16963, wherein the composition further comprises a visualization agent, and the visualization agent is, or comprises, iron oxide compound.

17164. The method of item 16963, wherein the composition further comprises a visualization agent, and the visualization agent is, or comprises, a dye, pigment, or colorant.

17165. The method of item 16963 wherein the agent is released in effective concentrations from the composition comprising the agent by diffusion over a period ranging from the time of administration to about 90 days.

17166. The method of item 16963 wherein the agent is released in effective concentrations from the composition comprising the agent by erosion of the composition over a period ranging from the time of administration to about 90 days.

17167. The method of item 16963 wherein the composition further comprises an inflammatory cytokine.

17168. The method of item 16963 wherein the composition further comprises an agent that stimulates cell proliferation.

17169. The method of item 16963 wherein the composition further comprises a polymeric carrier.

17170. The method of item 16963 wherein the composition is in the form of a gel, paste, or spray.

17171. The method of item 16963 wherein the implant is partially constructed with the agent or the composition.

17172. The method of item 16963 wherein the implant is fully constructed with the agent or the composition.

17173. The method of item 16963 wherein the implant is impregnated with the agent or the composition.

17174. The method of item 16963, wherein the agent or the composition forms a coating, and the coating directly contacts the implant.

17175. The method of item 16963, wherein the agent or the composition forms a coating, and the coating indirectly contacts the implant.

17176. The method of item 16963 wherein the agent or the composition forms a coating, and the coating partially covers the implant.

17177. The method of item 16963, wherein the agent or the composition forms a coating, and the coating completely covers the implant.

17178. The method of item 16963 wherein the agent or the composition is located within pores or holes of the implant.

17179. The method of item 16963 wherein the agent or the composition is located within a channel, lumen, or divet of the implant.

17180. The method of item 16963 wherein the implant further comprising an echogenic material.

17181. The method of item 16963 wherein the implant further comprises an echogenic material, wherein the echogenic material is in the form of a coating.

17182. The method of item 16963 wherein the implant is sterile.

17183. The method of item 16963 wherein the agent is delivered from the implant, wherein the agent is released into tissue in the vicinity of the implant after deployment of the implant.

17184. The method of item 16963 wherein the agent is delivered from the implant, wherein the agent is released into tissue in the vicinity of the implant after deployment of the implant, wherein the tissue is connective tissue.

17185. The method of item 16963 wherein the agent is delivered from the implant, wherein the agent is released into tissue in the vicinity of the implant after deployment of the implant, wherein the tissue is muscle tissue.

17186. The method of item 16963 wherein the agent is delivered from the implant, wherein the agent is released into tissue in the vicinity of the implant after deployment of the implant, wherein the tissue is nerve tissue.

17187. The method of item 16963 wherein the agent is delivered from the implant, wherein the agent is released into tissue in the vicinity of the implant after deployment of the implant, wherein the tissue is epithelium tissue.

17188. The method of item 16963 wherein the agent is delivered from the implant, wherein the agent is released in effective concentrations from the implant over a period ranging from the time of deployment of the implant to about 1 year.

17189. The method of item 16963 wherein the agent is delivered from the implant, wherein the agent is released in effective concentrations from the implant over a period ranging from about 1 month to 6 months.

17190. The method of item 16963 wherein the agent is delivered from the implant, wherein the agent is released in effective concentrations from the implant over a period ranging from about 1-90 days.

17191. The method of item 16963 wherein the agent is delivered from the implant, wherein the agent is released in effective concentrations from the implant at a constant rate.

17192. The method of item 16963 wherein the agent is delivered from the implant, wherein the agent is released in effective concentrations from the implant at an increasing rate.

17193. The method of item 16963 wherein the agent is delivered from the implant, wherein the agent is released in effective concentrations from the implant at a decreasing rate.

17194. The method of item 16963 wherein the agent is delivered from the implant, wherein the implant comprises about 0.01 μg to about 10 μg of the agent.

17195. The method of item 16963 wherein the agent is delivered from the implant, wherein the implant comprises about 10 μg to about 10 mg of the agent.

17196. The method of item 16963 wherein the agent is delivered from the implant, wherein the implant comprises about 10 mg to about 250 mg of the agent.

17197. The method of item 16963 wherein the agent is delivered from the implant, wherein the implant comprises about 250 mg to about 1000 mg of the agent.

17198. The method of item 16963 wherein the agent is delivered from the implant, wherein the implant comprises about 1000 mg to about 2500 mg of the agent.

17199. The method of item 16963 wherein the agent is delivered from the implant, wherein a surface of the implant comprises less than 0.01 μg of the agent per mm² of implant surface to which the agent is applied.

17200. The method of item 16963 wherein the agent is delivered from the implant, wherein a surface of the implant comprises about 0.01 μg to about 1 μg of the agent per mm² of implant surface to which the agent is applied.

17201. The method of item 16963 wherein the agent is delivered from the implant, wherein a surface of the implant comprises about 1 μg to about 10 μg of the agent per mm² of implant surface to which the agent is applied.

17202. The method of item 16963 wherein the agent is delivered from the implant, wherein a surface of the implant comprises about 10 μg to about 250 μg of the agent per mm² of implant surface to which the agent is applied.

17203. The method of item 16963 wherein the agent is delivered from the implant, wherein a surface of the implant comprises about 250 μg to about 1000 μg of the agent per mm² of implant surface to which the agent is applied.

17204. The method of item 16963 wherein the agent is delivered from the implant, wherein a surface of the implant comprises about 1000 μg to about 2500 μg of the agent per mm² of implant surface to which the agent is applied.

17205. The method of item 16963, wherein the implant further comprises a coating, and the coating is a uniform coating.

17206. The method of item 16963, wherein the implant further comprises a coating, and the coating is a non-uniform coating.

17207. The method of item 16963, wherein the implant further comprises a coating, and the coating is a discontinuous coating.

17208. The method of item 16963, wherein the implant further comprises a coating, and the coating is a patterned coating.

17209. The method of item 16963, wherein the implant further comprises a coating, and the coating has a thickness of 100 μm or less.

17210. The method of item 16963, wherein the implant further comprises a coating, and the coating has a thickness of 10 μm or less.

17211. The method of item 16963, wherein the implant further comprises a coating, and the coating adheres to the surface of the implant upon deployment of the implant.

17212. The method of item 16963, wherein the implant further comprises a coating, and the coating is stable at room temperature for a period of at least 1 year.

17213. The method of item 16963, wherein the implant further comprises a coating, and the agent is present in the coating in an amount ranging between about 0.0001% to about 1% by weight.

17214. The method of item 16963, wherein the implant further comprises a coating, and the agent is present in the coating in an amount ranging between about 1% to about 10% by weight.

17215. The method of item 16963, wherein the implant further comprises a coating, and the agent is present in the coating in an amount ranging between about 10% to about 25% by weight.

17216. The method of item 16963, wherein the implant further comprises a coating, and the agent is present in the coating in an amount ranging between about 25% to about 70% by weight.

17217. The method of item 16963, wherein the implant further comprises a coating, and the coating comprises a polymer.

17218. The method of item 16963, wherein the implant comprises a first coating having a first composition and a second coating having a second composition.

17219. The method of item 16963, wherein the implant comprises a first coating having a first composition and a second coating having a second composition, wherein the first composition and the second composition are different.

17220. The method of item 16963, wherein the implant is a total parenteral nutrition catheter.

17221. The method of item 16963, wherein the implant is a flow-directed balloon-tipped pulmonary artery catheter.

17222. A method of making a medical device comprising: combining a ventricular assist implant and an anti-scarring agent or a composition comprising an anti-scarring agent, wherein the agent inhibits scarring between the device and a host into which the device is implanted.

17223. The method of item 17222 wherein the agent inhibits cell regeneration.

17224. The method of item 17222 wherein the agent inhibits angiogenesis.

17225. The method of item 17222 wherein the agent inhibits fibroblast migration.

17226. The method of item 17222 wherein the agent inhibits fibroblast proliferation.

17227. The method of item 17222 wherein the agent inhibits deposition of extracellular matrix.

17228. The method of item 17222 wherein the agent inhibits tissue remodeling.

17229. The method of item 17222 wherein the agent is an angiogenesis inhibitor.

17230. The method of item 17222 wherein the agent is a 5-lipoxygenase inhibitor or antagonist.

17231. The method of item 17222 wherein the agent is a chemokine receptor antagonist.

17232. The method of item 17222 wherein the agent is a cell cycle inhibitor.

17233. The method of item 17222 wherein the agent is a taxane.

17234. The method of item 17222 wherein the agent is an anti-microtubule agent.

17235. The method of item 17222 wherein the agent is paclitaxel.

17236. The method of item 17222 wherein the agent is not paclitaxel.

17237. The method of item 17222 wherein the agent is an analogue or derivative of paclitaxel.

17238. The method of item 17222 wherein the agent is a vinca alkaloid.

17239. The method of item 17222 wherein the agent is camptothecin or an analogue or derivative thereof.

17240. The method of item 17222 wherein the agent is a podophyllotoxin.

17241. The method of item 17222 wherein the agent is a podophyllotoxin, wherein the podophyllotoxin is etoposide or an analogue or derivative thereof.

17242. The method of item 17222 wherein the agent is an anthracycline.

17243. The method of item 17222 wherein the agent is an anthracycline, wherein the anthracycline is doxorubicin or an analogue or derivative thereof.

17244. The method of item 17222 wherein the agent is an anthracycline, wherein the anthracycline is mitoxantrone or an analogue or derivative thereof.

17245. The method of item 17222 wherein the agent is a platinum compound.

17246. The method of item 17222 wherein the agent is a nitrosourea.

17247. The method of item 17222 wherein the agent is a nitroimidazole.

17248. The method of item 17222 wherein the agent is a folic acid antagonist.

17249. The method of item 17222 wherein the agent is a cytidine analogue.

17250. The method of item 17222 wherein the agent is a pyrimidine analogue.

17251. The method of item 17222 wherein the agent is a fluoropyrimidine analogue.

17252. The method of item 17222 wherein the agent is a purine analogue.

17253. The method of item 17222 wherein the agent is a nitrogen mustard or an analogue or derivative thereof.

17254. The method of item 17222 wherein the agent is a hydroxyurea.

17255. The method of item 17222 wherein the agent is a mytomicin or an analogue or derivative thereof.

17256. The method of item 17222 wherein the agent is an alkyl sulfonate.

17257. The method of item 17222 wherein the agent is a benzamide or an analogue or derivative thereof.

17258. The method of item 17222 wherein the agent is a nicotinamide or an analogue or derivative thereof.

17259. The method of item 17222 wherein the agent is a halogenated sugar or an analogue or derivative thereof.

17260. The method of item 17222 wherein the agent is a DNA alkylating agent.

17261. The method of item 17222 wherein the agent is an anti-microtubule agent.

17262. The method of item 17222 wherein the agent is a topoisomerase inhibitor.

17263. The method of item 17222 wherein the agent is a DNA cleaving agent.

17264. The method of item 17222 wherein the agent is an antimetabolite.

17265. The method of item 17222 wherein the agent inhibits adenosine deaminase.

17266. The method of item 17222 wherein the agent inhibits purine ring synthesis.

17267. The method of item 17222 wherein the agent is a nucleotide interconversion inhibitor.

17268. The method of item 17222 wherein the agent inhibits dihydrofolate reduction.

17269. The method of item 17222 wherein the agent blocks thymidine monophosphate.

17270. The method of item 17222 wherein the agent causes DNA damage.

17271. The method of item 17222 wherein the agent is a DNA intercalation agent.

17272. The method of item 17222 wherein the agent is a RNA synthesis inhibitor.

17273. The method of item 17222 wherein the agent is a pyrimidine synthesis inhibitor.

17274. The method of item 17222 wherein the agent inhibits ribonucleotide synthesis or function.

17275. The method of item 17222 wherein the agent inhibits thymidine monophosphate synthesis or function.

17276. The method of item 17222 wherein the agent inhibits DNA synthesis.

17277. The method of item 17222 wherein the agent causes DNA adduct formation.

17278. The method of item 17222 wherein the agent inhibits protein synthesis.

17279. The method of item 17222 wherein the agent inhibits microtubule function.

17280. The method of item 17222 wherein the agent is a cyclin dependent protein kinase inhibitor.

17281. The method of item 17222 wherein the agent is an epidermal growth factor kinase inhibitor.

17282. The method of item 17222 wherein the agent is an elastase inhibitor.

17283. The method of item 17222 wherein the agent is a factor Xa inhibitor.

17284. The method of item 17222 wherein the agent is a farnesyltransferase inhibitor.

17285. The method of item 17222 wherein the agent is a fibrinogen antagonist.

17286. The method of item 17222 wherein the agent is a guanylate cyclase stimulant.

17287. The method of item 17222 wherein the agent is a heat shock protein 90 antagonist.

17288. The method of item 17222 wherein the agent is a heat shock protein 90 antagonist, wherein the heat shock protein 90 antagonist is geldanamycin or an analogue or derivative thereof.

17289. The method of item 17222 wherein the agent is a guanylate cyclase stimulant.

17290. The method of item 17222 wherein the agent is a HMGCoA reductase inhibitor.

17291. The method of item 17222 wherein the agent is a HMGCoA reductase inhibitor, wherein the HMGCoA reductase inhibitor is simvastatin or an analogue or derivative thereof.

17292. The method of item 17222 wherein the agent is a hydroorotate dehydrogenase inhibitor.

17293. The method of item 17222 wherein the agent is an IKK2 inhibitor.

17294. The method of item 17222 wherein the agent is an IL-1 antagonist.

17295. The method of item 17222 wherein the agent is an ICE antagonist.

17296. The method of item 17222 wherein the agent is an IRAK antagonist.

17297. The method of item 17222 wherein the agent is an IL-4 agonist.

17298. The method of item 17222 wherein the agent is an immunomodulatory agent.

17299. The method of item 17222 wherein the agent is sirolimus or an analogue or derivative thereof.

17300. The method of item 17222 wherein the agent is not sirolimus.

17301. The method of item 17222 wherein the agent is everolimus or an analogue or derivative thereof.

17302. The method of item 17222 wherein the agent is tacrolimus or an analogue or derivative thereof.

17303. The method of item 17222 wherein the agent is not tacrolimus.

17304. The method of item 17222 wherein the agent is biolmus or an analogue or derivative thereof.

17305. The method of item 17222 wherein the agent is tresperimus or an analogue or derivative thereof.

17306. The method of item 17222 wherein the agent is auranofin or an analogue or derivative thereof.

17307. The method of item 17222 wherein the agent is 27-0-demethylrapamycin or an analogue or derivative thereof.

17308. The method of item 17222 wherein the agent is gusperimus or an analogue or derivative thereof.

17309. The method of item 17222 wherein the agent is pimecrolimus or an analogue or derivative thereof.

17310. The method of item 17222 wherein the agent is ABT-578 or an analogue or derivative thereof.

17311. The method of item 17222 wherein the agent is an inosine monophosphate dehydrogenase (IMPDH) inhibitor.

17312. The method of item 17222 wherein the agent is an IMPDH inhibitor, wherein the IMPDH inhibitor is mycophenolic acid or an analogue or derivative thereof.

17313. The method of item 17222 wherein the agent is an IMPDH inhibitor, wherein the IMPDH inhibitor is 1-alpha-25 dihydroxy vitamin D₃ or an analogue or derivative thereof.

17314. The method of item 17222 wherein the agent is a leukotriene inhibitor.

17315. The method of item 17222 wherein the agent is a MCP-1 antagonist.

17316. The method of item 17222 wherein the agent is a MMP inhibitor.

17317. The method of item 17222 wherein the agent is an NF kappa B inhibitor.

17318. The method of item 17222 wherein the agent is an NF kappa B inhibitor, wherein the NF kappa B inhibitor is Bay 11-7082.

17319. The method of item 17222 wherein the agent is an NO agonist.

17320. The method of item 17222 wherein the agent is a p38 MAP kinase inhibitor.

17321. The method of item 17222 wherein the agent is a p38 MAP kinase inhibitor, wherein the p38 MAP kinase inhibitor is SB 202190.

17322. The method of item 17222 wherein the agent is a phosphodiesterase inhibitor.

17323. The method of item 17222 wherein the agent is a TGF beta inhibitor.

17324. The method of item 17222 wherein the agent is a thromboxane A2 antagonist.

17325. The method of item 17222 wherein the agent is a TNFa antagonist.

17326. The method of item 17222 wherein the agent is a TACE inhibitor.

17327. The method of item 17222 wherein the agent is a tyrosine kinase inhibitor.

17328. The method of item 17222 wherein the agent is a vitronectin inhibitor.

17329. The method of item 17222 wherein the agent is a fibroblast growth factor inhibitor.

17330. The method of item 17222 wherein the agent is a protein kinase inhibitor.

17331. The method of item 17222 wherein the agent is a PDGF receptor kinase inhibitor.

17332. The method of item 17222 wherein the agent is an endothelial growth factor receptor kinase inhibitor.

17333. The method of item 17222 wherein the agent is a retinoic acid receptor antagonist.

17334. The method of item 17222 wherein the agent is a platelet derived growth factor receptor kinase inhibitor.

17335. The method of item 17222 wherein the agent is a fibronogin antagonist.

17336. The method of item 17222 wherein the agent is an antimycotic agent.

17337. The method of item 17222 wherein the agent is an antimycotic agent, wherein the antimycotic agent is sulconizole.

17338. The method of item 17222 wherein the agent is a bisphosphonate.

17339. The method of item 17222 wherein the agent is a phospholipase A1 inhibitor.

17340. The method of item 17222 wherein the agent is a histamine H1/H2/H3 receptor antagonist.

17341. The method of item 17222 wherein the agent is a macrolide antibiotic.

17342. The method of item 17222 wherein the agent is a GPIIb/IIIa receptor antagonist.

17343. The method of item 17222 wherein the agent is an endothelin receptor antagonist.

17344. The method of item 17222 wherein the agent is a peroxisome proliferator-activated receptor agonist.

17345. The method of item 17222 wherein the agent is an estrogen receptor agent.

17346. The method of item 17222 wherein the agent is a somastostatin analogue.

17347. The method of item 17222 wherein the agent is a neurokinin 1 antagonist.

17348. The method of item 17222 wherein the agent is a neurokinin 3 antagonist.

17349. The method of item 17222 wherein the agent is a VLA-4 antagonist.

17350. The method of item 17222 wherein the agent is an osteoclast inhibitor.

17351. The method of item 17222 wherein the agent is a DNA topoisomerase ATP hydrolyzing inhibitor.

17352. The method of item 17222 wherein the agent is an angiotensin I converting enzyme inhibitor.

17353. The method of item 17222 wherein the agent is an angiotensin II antagonist.

17354. The method of item 17222 wherein the agent is an enkephalinase inhibitor.

17355. The method of item 17222 wherein the agent is a peroxisome proliferator-activated receptor gamma agonist insulin sensitizer.

17356. The method of item 17222 wherein the agent is a protein kinase C inhibitor.

17357. The method of item 17222 wherein the agent is a ROCK (rho-associated kinase) inhibitor.

17358. The method of item 17222 wherein the agent is a CXCR3 inhibitor.

17359. The method of item 17222 wherein the agent is an Itk inhibitor.

17360. The method of item 17222 wherein the agent is a cytosolic phospholipase A₂-alpha inhibitor.

17361. The method of item 17222 wherein the agent is a PPAR agonist.

17362. The method of item 17222 wherein the agent is an immunosuppressant.

17363. The method of item 17222 wherein the agent is an Erb inhibitor.

17364. The method of item 17222 wherein the agent is an apoptosis agonist.

17365. The method of item 17222 wherein the agent is a lipocortin agonist.

17366. The method of item 17222 wherein the agent is a VCAM-1 antagonist.

17367. The method of item 17222 wherein the agent is a collagen antagonist.

17368. The method of item 17222 wherein the agent is an alpha 2 integrin antagonist.

17369. The method of item 17222 wherein the agent is a TNF alpha inhibitor.

17370. The method of item 17222 wherein the agent is a nitric oxide inhibitor.

17371. The method of item 17222 wherein the agent is a cathepsin inhibitor.

17372. The method of item 17222 wherein the agent is not an anti-inflammatory agent.

17373. The method of item 17222 wherein the agent is not a steroid.

17374. The method of item 17222 wherein the agent is not a glucocorticosteroid.

17375. The method of item 17222 wherein the agent is not dexamethasone.

17376. The method of item 17222 wherein the agent is not an anti-infective agent.

17377. The method of item 17222 wherein the agent is not an antibiotic.

17378. The method of item 17222 wherein the agent is not an anti-fungal agent.

17379. The method of item 17222, wherein the composition comprises a polymer.

17380. The method of item 17222, wherein the composition comprises a polymeric carrier.

17381. The method of item 17222 wherein the anti-scarring agent inhibits adhesion between the device and a host into which the device is implanted.

17382. The method of item 17222 wherein the device delivers the anti-scarring agent locally to tissue proximate to the device.

17383. The method of item 17222 wherein the device has a coating that comprises the anti-scarring agent.

17384. The method of item 17222, wherein the device has a coating that comprises the agent and is disposed on a surface of the implant.

17385. The method of item 17222, wherein the device has a coating that comprises the agent and directly contacts the implant.

17386. The method of item 17222, wherein the device has a coating that comprises the agent and indirectly contacts the implant.

17387. The method of item 17222, wherein the device has a coating that comprises the agent and partially covers the implant.

17388. The method of item 17222, wherein the device has a coating that comprises the agent and completely covers the implant.

17389. The method of item 17222, wherein the device has a uniform coating.

17390. The method of item 17222, wherein the device has a non-uniform coating.

17391. The method of item 17222, wherein the device has a discontinuous coating.

17392. The method of item 17222, wherein the device has a patterned coating.

17393. The method of item 17222, wherein the device has a coating with a thickness of 100 μm or less.

17394. The method of item 17222, wherein the device has a coating with a thickness of 10 μm or less.

17395. The method of item 17222, wherein the device has a coating, and the coating adheres to the surface of the implant upon deployment of the implant.

17396. The method of item 17222, wherein the device has a coating, and wherein the coating is stable at room temperature for a period of 1 year.

17397. The method of item 17222, wherein the device has a coating, and wherein the anti-scarring agent is present in the coating in an amount ranging between about 0.0001% to about 1% by weight.

17398. The method of item 17222, wherein the device has a coating, and wherein the anti-scarring agent is present in the coating in an amount ranging between about 1% to about 10% by weight.

17399. The method of item 17222, wherein the device has a coating, and wherein the anti-scarring agent is present in the coating in an amount ranging between about 10% to about 25% by weight.

17400. The method of item 17222, wherein the device has a coating, and wherein the anti-scarring agent is present in the coating in an amount ranging between about 25% to about 70% by weight.

17401. The method of item 17222, wherein the device has a coating, and wherein the coating further comprises a polymer.

17402. The method of item 17222, wherein the device has a first coating having a first composition and a second coating having a second composition.

17403. The method of item 17222, wherein the device has a first coating having a first composition and a second coating having a second composition, wherein the first composition and the second composition are different.

17404. The method of item 17222, wherein the composition comprises a polymer.

17405. The method of item 17222, wherein the composition comprises a polymeric carrier.

17406. The method of item 17222, wherein the composition comprises a polymeric carrier, and wherein the polymeric carrier comprises a copolymer.

17407. The method of item 17222, wherein the composition comprises a polymeric carrier, and wherein the polymeric carrier comprises a block copolymer.

17408. The method of item 17222, wherein the composition comprises a polymeric carrier, and wherein the polymeric carrier comprises a random copolymer.

17409. The method of item 17222, wherein the composition comprises a polymeric carrier, and wherein the polymeric carrier comprises a biodegradable polymer.

17410. The method of item 17222, wherein the composition comprises a polymeric carrier, and wherein the polymeric carrier comprises a non-biodegradable polymer.

17411. The method of item 17222, wherein the composition comprises a polymeric carrier, and wherein the polymeric carrier comprises a hydrophilic polymer.

17412. The method of item 17222, wherein the composition comprises a polymeric carrier, and wherein the polymeric carrier comprises a hydrophobic polymer.

17413. The method of item 17222, wherein the composition comprises a polymeric carrier, and wherein the polymeric carrier comprises a polymer having hydrophilic domains.

17414. The method of item 17222, wherein the composition comprises a polymeric carrier, and wherein the polymeric carrier comprises a polymer having hydrophobic domains.

17415. The method of item 17222, wherein the composition comprises a polymeric carrier, and wherein the polymeric carrier comprises a non-conductive polymer.

17416. The method of item 17222, wherein the composition comprises a polymeric carrier, and wherein the polymeric carrier comprises an elastomer.

17417. The method of item 17222, wherein the composition comprises a polymeric carrier, and wherein the polymeric carrier comprises a hydrogel.

17418. The method of item 17222, wherein the composition comprises a polymeric carrier, and wherein the polymeric carrier comprises a silicone polymer.

17419. The method of item 17222, wherein the composition comprises a polymeric carrier, and wherein the polymeric carrier comprises a hydrocarbon polymer.

17420. The method of item 17222, wherein the composition comprises a polymeric carrier, and wherein the polymeric carrier comprises a styrene-derived polymer.

17421. The method of item 17222, wherein the composition comprises a polymeric carrier, and wherein the polymeric carrier comprises a butadiene polymer.

17422. The method of item 17222, wherein the composition comprises a polymeric carrier, and wherein the polymeric carrier comprises a macromer.

17423. The method of item 17222, wherein the composition comprises a polymeric carrier, and wherein the polymeric carrier comprises a poly(ethylene glycol)polymer.

17424. The method of item 17222 wherein the composition comprises a polymeric carrier, and wherein the polymeric carrier comprises an amorphous polymer.

17425. The method of item 17222, wherein the device comprises a lubricious coating.

17426. The method of item 17222 wherein the anti-scarring agent is located within pores or holes of the device.

17427. The method of item 17222 wherein the anti-scarring agent is located within a channel, lumen, or divet of the device.

17428. The method of item 17222, wherein the device comprises a second pharmaceutically active agent.

17429. The method of item 17222 wherein the device comprises an anti-inflammatory agent.

17430. The method of item 17222 wherein the device comprises an agent that inhibits infection.

17431. The method of item 17222 wherein the device comprises an agent that inhibits infection, and wherein the agent is an anthracycline.

17432. The method of item 17222 wherein the device comprises an agent that inhibits infection, and wherein the agent is doxorubicin.

17433. The method of item 17222 wherein the device comprises an agent that inhibits infection, and wherein the agent is mitoxantrone.

17434. The method of item 17222 wherein the device comprises an agent that inhibits infection, and wherein the agent is a fluoropyrimidine.

17435. The method of item 17222 wherein the device comprises an agent that inhibits infection, and wherein the agent is 5-fluorouracil (5-FU).

17436. The method of item 17222 wherein the device comprises an agent that inhibits infection, and wherein the agent is a folic acid antagonist.

17437. The method of item 17222 wherein the device comprises an agent that inhibits infection, and wherein the agent is methotrexate.

17438. The method of item 17222 wherein the device comprises an agent that inhibits infection, and wherein the agent is a podophylotoxin.

17439. The method of item 17222 wherein the device comprises an agent that inhibits infection, and wherein the agent is etoposide.

17440. The method of item 17222 wherein the device comprises an agent that inhibits infection, and wherein the agent is a camptothecin.

17441. The method of item 17222 wherein the device comprises an agent that inhibits infection, and wherein the agent is a hydroxyurea.

17442. The method of item 17222 wherein the device comprises an agent that inhibits infection, and wherein the agent is a platinum complex.

17443. The method of item 17222 wherein the device comprises an agent that inhibits infection, and wherein the agent is cisplatin.

17444. The method of item 17222, further comprising an anti-thrombotic agent.

17445. The method of item 17222 wherein the device comprises a visualization agent.

17446. The method of item 17222 wherein the device comprises a visualization agent, wherein the visualization agent is a radiopaque material, and wherein the radiopaque material comprises a metal, a halogenated compound, or a barium containing compound.

17447. The method of item 17222 wherein the device comprises a visualization agent, wherein the visualization agent is a radiopaque material, and wherein the radiopaque material comprises barium, tantalum, or technetium.

17448. The method of item 17222 wherein the device comprises a visualization agent, and wherein the visualization agent is a MRI responsive material.

17449. The method of item 17222 wherein the device comprises a visualization agent, and wherein the visualization agent comprises a gadolinium chelate.

17450. The method of item 17222 wherein the device comprises a visualization agent, and wherein the visualization agent comprises iron, magnesium, manganese, copper, or chromium.

17451. The method of item 17222 wherein the device comprises a visualization agent, and wherein the visualization agent comprises an iron oxide compound.

17452. The method of item 17222 wherein the device comprises a visualization agent, and wherein the visualization agent comprises a dye, pigment, or colorant.

17453. The method of item 17222 wherein the device comprises an echogenic material.

17454. The method of item 1.7222 wherein the device comprises an echogenic material, and wherein the echogenic material is in the form of a coating.

17455. The method of item 17222 wherein the device is sterile.

17456. The method of item 17222 wherein the anti-scarring agent is released into tissue in the vicinity of the device after deployment of the device.

17457. The method of item 17222 wherein the anti-scarring agent is released into tissue in the vicinity of the device after deployment of the device, and wherein the tissue is connective tissue.

17458. The method of item 17222 wherein the anti-scarring agent is released into tissue in the vicinity of the device after deployment of the device, and wherein the tissue is muscle tissue.

17459. The method of item 17222 wherein the anti-scarring agent is released into tissue in the vicinity of the device after deployment of the device, and wherein the tissue is nerve tissue.

17460. The method of item 17222 wherein the anti-scarring agent is released into tissue in the vicinity of the device after deployment of the device, and wherein the tissue is epithelium tissue.

17461. The method of item 17222 wherein the anti-scarring agent is released in effective concentrations from the device over a period ranging from the time of deployment of the device to about 1 year.

17462. The method of item 17222 wherein the anti-scarring agent is released in effective concentrations from the device over a period ranging from about 1 month to 6 months.

17463. The method of item 17222 wherein the anti-scarring agent is released in effective concentrations from the device over a period ranging from about 1-90 days.

17464. The method of item 17222 wherein the anti-scarring agent is released in effective concentrations from the device at a constant rate.

17465. The method of item 17222 wherein the anti-scarring agent is released in effective concentrations from the device at an increasing rate.

17466. The method of item 17222 wherein the anti-scarring agent is released in effective concentrations from the device at a decreasing rate.

17467. The method of item 17222 wherein the anti-scarring agent is released in effective concentrations from the composition comprising the anti-scarring agent by diffusion over a period ranging from the time of deployment of the device to about 90 days.

17468. The method of item 17222 wherein the anti-scarring agent is released in effective concentrations from the composition comprising the anti-scarring agent by erosion of the composition over a period ranging from the time of deployment of the device to about 90 days.

17469. The method of item 17222 wherein the device comprises about 0.01 μg to about 10 μg of the anti-scarring agent.

17470. The method of item 17222 wherein the device comprises about 10 μg to about 10 mg of the anti-scarring agent.

17471. The method of item 17222 wherein the device comprises about 10 mg to about 250 mg of the anti-scarring agent.

17472. The method of item 17222 wherein the device comprises about 250 mg to about 1000 mg of the anti-scarring agent.

17473. The method of item 17222 wherein the device comprises about 1000 mg to about 2500 mg of the anti-scarring agent.

17474. The method of item 17222 wherein a surface of the device comprises less than 0.01 μg of the anti-scarring agent per mm² of device surface to which the anti-scarring agent is applied.

17475. The method of item 17222 wherein a surface of the device comprises about 0.01 μg to about 1 μg of the anti-scarring agent per mm² of device surface to which the anti-scarring agent is applied.

17476. The method of item 17222 wherein a surface of the device comprises about 1 μg to about 10 μg of the anti-scarring agent per mm² of device surface to which the anti-scarring agent is applied.

17477. The method of item 17222 wherein a surface of the device comprises about 10 μg to about 250 μg of the anti-scarring agent per mm² of device surface to which the anti-scarring agent is applied.

17478. The method of item 17222 wherein a surface of the device comprises about 250 μg to about 1000 μg of the anti-scarring agent of anti-scarring agent per mm² of device surface to which the anti-scarring agent is applied.

17479. The method of item 17222 wherein a surface of the device comprises about 1000 μg to about 2500 μg of the anti-scarring agent per mm² of device surface to which the anti-scarring agent is applied.

17480. The method of item 17222 wherein the combining is performed by direct affixing the agent or the composition to the implant.

17481. The method of item 17222 wherein the combining is performed by spraying the agent or the component onto the implant.

17482. The method of item 17222 wherein the combining is performed by electrospraying the agent or the composition onto the implant.

17483. The method of item 17222 wherein the combining is performed by dipping the implant into a solution comprising the agent or the composition.

17484. The method of item 17222 wherein the combining is performed by covalently attaching the agent or the composition to the implant.

17485. The method of item 17222 wherein the combining is performed by non-covalently attaching the agent or the composition to the implant.

17486. The method of item 17222 wherein the combining is performed by coating the implant with a substance that contains the agent or the composition.

17487. The method of item 17222 wherein the combining is performed by coating the implant with a substance that absorbs the agent.

17488. The method of item 17222 wherein the combining is performed by interweaving a thread composed of, or coated with, the agent or the composition.

17489. The method of item 17222 wherein the combining is performed by covering all the implant with a sleeve that contains the agent or the composition.

17490. The method of item 17222 wherein the combining is performed by covering a portion of the implant with a sleeve that contains the agent or the composition.

17491. The method of item 17222 wherein the combining is performed by covering all the implant with a cover that contains the agent or the composition.

17492. The method of item 17222 wherein the combining is performed by covering a portion of the implant with a cover that contains the agent or the composition.

17493. The method of item 17222 wherein the combining is performed by covering all the implant with an electrospun fabric that contains the agent or the composition.

17494. The method of item 17222 wherein the combining is performed by covering a portion of the implant with an electrospun fabric that contains the agent or the composition.

17495. The method of item 17222 wherein the combining is performed by covering all the implant with a mesh that contains the agent or the composition.

17496. The method of item 17222 wherein the combining is performed by covering a portion of the implant with a mesh that contains the agent or the composition.

17497. The method of item 17222 wherein the combining is performed by constructing all the implant with the agent or the composition.

17498. The method of item 17222 wherein the combining is performed by constructing a portion of the implant with the agent or the composition.

17499. The method of item 17222 wherein the combining is performed by impregnating the implant with the agent or the composition.

17500. The method of item 17222 wherein the combining is performed by constructing all of the implant from a degradable polymer that releases the agent.

17501. The method of item 17222 wherein the combining is performed by constructing a portion of the implant from a degradable polymer that releases the agent.

17502. The method of item 17222 wherein the combining is performed by dipping the implant into a solution that comprise the agent and an inert solvent for the implant.

17503. The method of item 17222 wherein the combining is performed by dipping the implant into a solution that comprises the agent and a solvent that will swill the implant.

17504. The method of item 17222 wherein the combining is performed by dipping the implant into a solution that comprises the agent and a solvent that will dissolve the implant.

17505. The method of item 17222 wherein the combining is performed by dipping the implant into a solution that comprises the agent, a polymer and an inert solvent for the implant.

17506. The method of item 17222 wherein the combining is performed by dipping the implant into a solution that comprises the agent, a polymer and a solvent that will swill the implant.

17507. The method of item 17222 wherein the combining is performed by dipping the implant into a solution that comprises the agent, a polymer and a solvent that will dissolve the implant.

17508. The method of item 17222 wherein the combining is performed by spraying the implant into a solution that comprises the agent and an inert solvent for the implant.

17509. The method of item 17222 wherein the combining is performed by spraying the implant into a solution that comprises the agent and a solvent that will swill the implant.

17510. The method of item 17222 wherein the combining is performed by spraying the implant into a solution that comprises the agent and a solvent that will dissolve the implant.

17511. The method of item 17222 wherein the combining is performed by spraying the implant into a solution that comprises the agent, a polymer and an inert solvent for the implant.

17512. The method of item 17222 wherein the combining is performed by spraying the implant into a solution that comprises the agent, a polymer and a solvent that will swill the implant.

17513. The method of item 17222 wherein the combining is performed by spraying the implant into a solution that comprises the agent, a polymer and a solvent that will dissolve the implant.

17514. The method of item 17222 wherein the implant is a left ventricular assist device.

17515. The method of item 17222 wherein the implant is a right ventricular assist device.

17516. The method of item 17222 wherein the implant is a biventricular assist device.

17517. The method of item 17222 wherein the implant is a cardiac assist device.

All of the above U.S. patents, U.S. patent application publications, U.S. patent applications, foreign patents, foreign patent applications and non-patent publications referred to in this specification and/or listed in the Application Data Sheet, are incorporated herein by reference, in their entirety.

From the foregoing it will be appreciated that, although specific embodiments of the invention have been described herein for purposes of illustration, various modifications may be made without deviating from the spirit and scope of the invention. Accordingly, the invention is not limited except as by the appended claims.

1-4220. (canceled)

4221. A device, comprising a pressure monitoring implant and an anti-scarring agent or a composition comprising an anti-scarring agent, wherein the agent inhibits scarring between the device and a host into which the device is implanted.

4222. The device of claim 4221 wherein the agent inhibits cell regeneration.

4223. The device of claim 4221 wherein the agent inhibits angiogenesis.

4224. The device of claim 4221 wherein the agent inhibits fibroblast migration.

4225. The device of claim 4221 wherein the agent inhibits fibroblast proliferation.

4226. The device of claim 4221 wherein the agent inhibits deposition of extracellular matrix.

4227. The device of claim 4221 wherein the agent inhibits tissue remodeling.

4228. (canceled)

4229. The device of claim 4221 wherein the agent is a 5-lipoxygenase inhibitor or antagonist.

4230. The device of claim 4221 wherein the agent is a chemokine receptor antagonist.

4231. The device of claim 4221 wherein the agent is a cell cycle inhibitor.

4232. The device of claim 4221 wherein the agent is a taxane.

4233. The device of claim 4221 wherein the agent is an anti-microtubule agent.

4234.-4236. (canceled)

4237. The device of claim 4221 wherein the agent is a vinca alkaloid.

4238. (canceled)

4239. The device of claim 4221 wherein the agent is a podophyllotoxin.

4240. (canceled)

4241. The device of claim 4221 wherein the agent is an anthracycline.

4242-4253. (canceled)

4254. The device of claim 4221 wherein the agent is a mytomicin or an analogue or derivative thereof.

4255-4258. (canceled)

4259. The device of claim 4221 wherein the agent is a DNA alkylating agent.

4260. (canceled)

4261. (canceled)

4262. The device of claim 4221 wherein the agent is a DNA cleaving agent.

4263-4270. (canceled)

4271. The device of claim 4221 wherein the agent is a RNA synthesis inhibitor.

4272-4276. (canceled)

4277. The device of claim 4221 wherein the agent inhibits protein synthesis.

4278-4285. (canceled)

4286. The device of claim 4221 wherein the agent is a heat shock protein 90 antagonist.

4287. (canceled)

4288. (canceled)

4289. The device of claim 4221 wherein the agent is a HMGCoA reductase inhibitor.

4290. (canceled)

4291. (canceled)

4292. The device of claim 4221 wherein the agent is an IKK2 inhibitor.

4293. The device of claim 4221 wherein the agent is an IL-1 antagonist.

4294. The device of claim 4221 wherein the agent is an ICE antagonist.

4295. The device of claim 4221 wherein the agent is an IRAK antagonist.

4296. (canceled)

4297. The device of claim 4221 wherein the agent is an immunomodulatory agent.

4298-4309. (canceled)

4310. The device of claim 4221 wherein the agent is an inosine monophosphate dehydrogenase (IMPDH) inhibitor.

4311. (canceled)

4312. (canceled)

4313. The device of claim 4221 wherein the agent is a leukotriene inhibitor.

4314. (canceled)

4315. (canceled)

4316. The device of claim 4221 wherein the agent is an NF kappa B inhibitor.

4317. (canceled)

4318. (canceled)

4319. The device of claim 4221 wherein the agent is a p38 MAP kinase inhibitor.

4320-4426. (canceled)

4427. The device of claim 4221, further comprising a second pharmaceutically active agent.

4428. (canceled)

4429. The device of claim 4221, further comprising an agent that inhibits infection.

4430-9621. (canceled)

9622. A method for inhibiting scarring comprising placing a pressure monitoring implant and an anti-scarring agent or a composition comprising an anti-scarring agent into an animal host, wherein the agent inhibits scarring.

9623. The method of claim 9622 wherein the agent inhibits cell regeneration.

9624. The method of claim 9622 wherein the agent inhibits angiogenesis.

9625. The method of claim 9622 wherein the agent inhibits fibroblast migration.

9626. The method of claim 9622 wherein the agent inhibits fibroblast proliferation.

9627. The method of claim 9622 wherein the agent inhibits deposition of extracellular matrix.

9628. The method of claim 9622 wherein the agent inhibits tissue remodeling.

9629. (canceled)

9630. The method of claim 9622 wherein the agent is a 5-lipoxygenase inhibitor or antagonist.

9631. The method of claim 9622 wherein the agent is a chemokine receptor antagonist.

9632. The method of claim 9622 wherein the agent is a cell cycle inhibitor.

9633. The method of claim 9622 wherein the agent is a taxane.

9634. The method of claim 9622 wherein the agent is an anti-microtubule agent.

9635-9637. (canceled)

9638. The method of claim 9622 wherein the agent is a vinca alkaloid.

9639. (canceled)

9640. The method of claim 9622 wherein the agent is a podophyllotoxin.

9641. (canceled)

9642. The method of claim 9622 wherein the agent is an anthracycline.

9643-9654. (canceled)

9655. The method of claim 9622 wherein the agent is a mytomicin or an analogue or derivative thereof.

9656-9659. (canceled)

9660. The method of claim 9622 wherein the agent is a DNA alkylating agent.

9661. (canceled)

9662. (canceled)

9663. The method of claim 9622 wherein the agent is a DNA cleaving agent.

9664-9671. (canceled)

9672. The method of claim 9622 wherein the agent is a RNA synthesis inhibitor.

9673-9677. (canceled)

9678. The method of claim 9622 wherein the agent inhibits protein synthesis.

9679.-9686. (canceled)

9687. The method of claim 9622 wherein the agent is a heat shock protein 90 antagonist.

9688. (canceled)

9689. (canceled)

9690. The method of claim 9622 wherein the agent is a HMGCoA reductase inhibitor.

9691. (canceled)

9692. (canceled)

9693. The method of claim 9622 wherein the agent is an IKK2 inhibitor.

9694. The method of claim 9622 wherein the agent is an IL-1 antagonist.

9695. The method of claim 9622 wherein the agent is an ICE antagonist.

9696. The method of claim 9622 wherein the agent is an IRAK antagonist.

9697. (canceled)

9698. The method of claim 9622 wherein the agent is an immunomodulatory agent.

9699-9710. (canceled)

9711. The method of claim 9622 wherein the agent is an inosine monophosphate dehydrogenase (IMPDH) inhibitor.

9712. (canceled)

9713. (canceled)

9714. The method of claim 9622 wherein the agent is a leukotriene inhibitor.

9715. (canceled)

9716. (canceled)

9717. The method of claim 9622 wherein the agent is an NF kappa B inhibitor.

9718. (canceled)

9719. (canceled)

9720. The method of claim 9622 wherein the agent is a p38 MAP kinase inhibitor.

9721-9798. (canceled)

9799. The method of claim 9622, wherein the composition further comprises a second pharmaceutically active agent.

9800. (canceled)

9801. The method of claim 9622, wherein the composition further comprises an agent that inhibits infection.

9802-15658. (canceled)

15659. A method of making a medical device comprising: combining a pressure monitoring implant and an anti-scarring agent or a composition comprising an anti-scarring agent, wherein the agent inhibits scarring between the device and a host into which the device is implanted.

15660. The method of claim 15659 wherein the agent inhibits cell regeneration.

15661. The method of claim 15659 wherein the agent inhibits angiogenesis.

15662. The method of claim 15659 wherein the agent inhibits fibroblast migration.

15663. The method of claim 15659 wherein the agent inhibits fibroblast proliferation.

15664. The method of claim 15659 wherein the agent inhibits deposition of extracellular matrix.

15665. The method of claim 15659 wherein the agent inhibits tissue remodeling.

15666. (canceled)

15667. The method of claim 15659 wherein the agent is a 5-lipoxygenase inhibitor or antagonist.

15668. The method of claim 15659 wherein the agent is a chemokine receptor antagonist.

15669. The method of claim 15659 wherein the agent is a cell cycle inhibitor.

15670. The method of claim 15659 wherein the agent is a taxane.

15671. The method of claim 15659 wherein the agent is an anti-microtubule agent.

15672-15674. (canceled)

15675. The method of claim 15659 wherein the agent is a vinca alkaloid.

15676. (canceled)

15677. The method of claim 15659 wherein the agent is a podophyllotoxin.

15678. (canceled)

15679. The method of claim 15659 wherein the agent is an anthracycline.

15680-15691. (canceled)

15692. The method of claim 15659 wherein the agent is a mytomicin or an analogue or derivative thereof.

15693-15696. (canceled)

15697. The method of claim 15659 wherein the agent is a DNA alkylating agent.

15698. (canceled)

15699. (canceled)

15700. The method of claim 15659 wherein the agent is a DNA cleaving agent.

15701-15708. (canceled)

15709. The method of claim 15659 wherein the agent is a RNA synthesis inhibitor.

15710-15714. (canceled)

15715. The method of claim 15659 wherein the agent inhibits protein synthesis.

15716-15723. (canceled)

15724. The method of claim 15659 wherein the agent is a heat shock protein 90 antagonist.

15725. (canceled)

15726. (canceled)

15727. The method of claim 15659 wherein the agent is a HMGCoA reductase inhibitor.

15728. (canceled)

15729. (canceled)

15730. The method of claim 15659 wherein the agent is an IKK2 inhibitor.

15731. The method of claim 15659 wherein the agent is an IL-1 antagonist.

15732. The method of claim 15659 wherein the agent is an ICE antagonist.

15733. The method of claim 15659 wherein the agent is an IRAK antagonist.

15734. (canceled)

15735. The method of claim 15659 wherein the agent is an immunomodulatory agent.

15736-15747. (canceled)

15748. The method of claim 15659 wherein the agent is an inosine monophosphate dehydrogenase (IMPDH) inhibitor.

15749. (canceled)

15750. (canceled)

15751. The method of claim 15659 wherein the agent is a leukotriene inhibitor.

15752. (canceled)

15753. (canceled)

15754. The method of claim 15659 wherein the agent is an NF kappa B inhibitor.

15755. (canceled)

15756. (canceled)

15757. The method of claim 15659 wherein the agent is a p38 MAP kinase inhibitor.

15758-15864. (canceled)

15865. The method of claim 15659, wherein the device comprises a second pharmaceutically active agent.

15866. (canceled)

15867. The method of claim 15659 wherein the device comprises an agent that inhibits infection.

15868-17517. (canceled)
